(19) United States
(12) Patent Application Publication
(10) Pub. No.: US 2026/0200947 A1
(43) Pub. Date: Jul. 16, 2026

(54) FUSED CYCLIC COMPOUNDS AND USE THEREOF

(71) Applicant: Jacobio Pharmaceuticals Co., Ltd., Beijing (CN)

(72) Inventors: Hongwei YANG, Beijing (CN); Cunbo MA, Beijing (CN); Peng WANG, Beijing (CN); Panliang GAO, Beijing (CN); Huifeng HAN, Beijing (CN); Hao ZHANG, Beijing (CN); Runze LI, Beijing (CN); Xiaoyu LIU, Beijing (CN); Yanping WANG, Beijing (CN); Wei LONG, Beijing (CN)

(21) Appl. No.: 19/136,115

(22) PCT Filed: Dec. 6, 2023
(86) PCT No.: PCT/CN2023/136747

§ 371 Date: Jun. 5, 2025

(30) Foreign Application Priority Data
Dec. 7, 2022 (WO) PCT/CN2022/137156
Jan. 10, 2023 (WO) PCT/CN2023/071719
Mar. 29, 2023 (WO) PCT/CN2023/084883
Apr. 14, 2023 (WO) PCT/CN2023/088433
Jun. 20, 2023 (WO) PCT/CN2023/101375
Sep. 1, 2023 (WO) PCT/CN2023/116529
Sep. 15, 2023 (WO) PCT/CN2023/119147
Oct. 16, 2023 (WO) PCT/CN2023/124703
Nov. 2, 2023 (WO) PCT/CN2023/129225
Nov. 15, 2023 (WO) PCT/CN2023/131834
Nov. 29, 2023 (WO) PCT/CN2023/135192

(51) Int. Cl. C07D 519/00; A61K 31/519; A61K 31/5383; A61K 31/55; A61K 31/553; A61P 35/00; C07B 59/00; C07D 498/22

(52) U.S. Cl. CPC C07D 519/00; A61K 31/519; A61K 31/5383; A61K 31/55; A61K 31/553; A61P 35/00; C07B 59/002; C07D 498/22

(57) ABSTRACT

The invention relates to fused cyclic compounds, a composition containing the same and the use thereof.

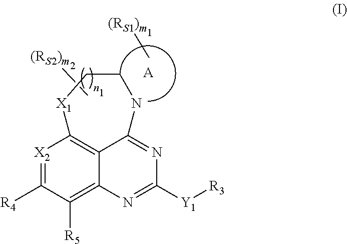

### TECHNICAL FIELD

The invention relates to compounds that inhibit the activity of multiple forms of K-Ras protein including K-Ras wild type and K-Ras mutant types, compositions comprising the same, and the methods of using the same.

### BACKGROUND ART

There are unmet needs to develop new multiple K-Ras inhibitors for treating K-Ras mediated cancers.

### SUMMARY OF THE INVENTION

Provided herein is a compound of formula (I):

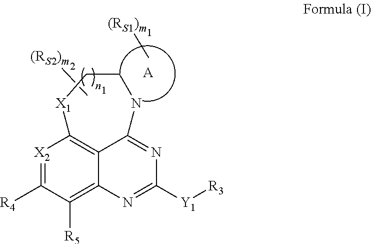

a stereoisomer thereof, a pharmaceutically acceptable salt thereof, a pharmaceutically acceptable salt of the stereoisomer thereof, a prodrug thereof, a deuterated molecule thereof or a PROTAC molecule thereof;

Wherein, the definition of each of variables is as below.

Also provided herein is a pharmaceutical composition comprising a therapeutically effective amount of the compound as defined herein, and a pharmaceutically acceptable excipient.

Also provided herein is a method for treating cancer in a subject comprising administering a therapeutically effective amount of the compound, or the pharmaceutical composition as defined herein to a subject in need thereof.

Also provided herein is a method for treating cancer in a subject in need thereof, the method comprising (a) determining whether the cancer is associated with K-Ras G12C, K-Ras G12D, K-Ras G12V, K-Ras G13D, K-Ras G12R, K-Ras G12S, K-Ras G12A, K-Ras Q61H mutation and/or K-Ras wild type amplification; and (b) if so, administering a therapeutically effective amount of the compound, or the pharmaceutical composition as defined herein to the subject in need thereof.

Also provided herein is the compound, or the pharmaceutical composition as defined herein for use in therapy.

Also provided herein is the compound, or the pharmaceutical composition as defined herein for use as a medicament.

Also provided herein is the compound, or the pharmaceutical composition as defined herein for use in a method for the treatment of cancer.

Also provided herein is a use of the compound, or the pharmaceutical composition as defined herein for the treatment of cancer.

Also provided herein is a use of the compound, or the pharmaceutical composition as defined herein for the manufacture of a medicament for the treatment of cancer.

### DETAILED DESCRIPTION

Provided herein are the following disclosures:

[1]. A compound of formula (I):

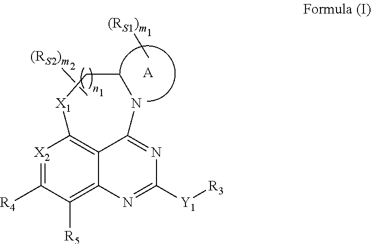

a stereoisomer thereof, a pharmaceutically acceptable salt thereof, a pharmaceutically acceptable salt of the stereoisomer, a prodrug thereof, a deuterated molecule thereof or a PROTAC molecule thereof;
Wherein,
X₁ at each occurrence is independently a bond, —C(R_{X11})(R_{X12})—, —NR_{X13}—, —O—, —S—, —S(═O)— or —S(═O)₂—;
R_{X11} or R_{X12} is independently hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, R_{X11} and R_{X12} together with the carbon atom to which they are both attached form

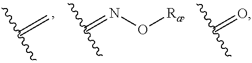

a 3-10 membered carbocyclic ring or a 3-10 heterocyclic ring; wherein, said

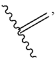

3-10 membered carbocyclic ring or 3-10 heterocyclic ring is independently unsubstituted or substituted with one or more R_{SX1};
R_{X13} is hydrogen, deuterium, —C₁₋₆ alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
X₂ at each occurrence is independently N or CR₁;
R₁ is hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{1A})₂, —OR₁A, —SR_{1A}, —S(═O)R_{1B}, —S(═O)₂R_{1B}, —C(═O)R_{1B}, —C(═O)OR_{1A}, —OC(═O)R_{1B}, —C(═O)N(R_{1A})₂, —NR_{1A}C(═O)R_{1B}, —OC(═O)OR_{1A}, —NR_{1A}C(═O)OR_{1A}, —NR_{1A}C(═S)OR_{1A}, —OC(═O)N(R_{1A})₂, —NR_{1A}C(═O)N(R_{1A})₂, —S(═O)OR_{1A}, —OS(═O)R_{1B}, —S(═O)N(R_{1A})₂, —NR_{1A}S(═O)R_{1B}, —S(═O)₂OR_{1A}, —OS(═O)₂R_{1B}, —S(═O)₂N(R_{1A})₂, —NR_{1A}S(═O)₂R_{1B}, —OS(═O)₂OR_{1A}, —NR_{1A}S(═O)₂OR_{1A}, —OS(═O)₂N(R_{1A})₂, —NR_{1A}S(═O)₂N(R_{1A})₂, —P(R_{1A})₂, —P(═O)(R_{1B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{1C})₂, —OR_{1C}, —SR_{1C}, —S(═O)R_{1D}, —S(═O)₂R_{1D}, —C(═O)R_{1D}, —C(═O)OR_{1D}, —OC(═O)R_{1D}, —C(═O)N(R_{1C})₂, —NR_{1C}C(═O)R_{1D}, —OC(═O)OR_{1C}, —NR_{1C}C(═O)OR_{1C}, —NR_{1C}C(═S)OR_{1C}, —OC(═O)N(R_{1C})₂, —NR_{1C}C(═O)N(R_{1C})₂, —S(═O)OR_{1C}, —OS(═O)R_{1D}, —S(═O)N(R_{1C})₂, —NR_{1C}S(═O)R_{1D}, —S(═O)₂OR_{1C}, —OS(═O)₂R_{1D}, —S(═O)₂N(R_{1C})₂, —NR_{1C}S(═O)₂R_{1D}, —OS(═O)₂OR_{1C}, —NR_{1C}S(═O)₂OR_{1C}, —OS(═O)₂N(R_{1C})₂, —NR_{1C}S(═O)₂N(R_{1C})₂, —P(R_{1C})₂, —P(═O)(R_{1D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
n₁ is 0, 1, 2, 3, 4, 5, or 6;
Ring A is a 3-20 membered heterocyclic ring only comprising the N atom attached to the pyrimidine ring, or a 3-20 (such as 3-10) membered heterocyclic ring comprising one or more additional heteroatoms selected from O, S, S═O or S(═O)₂ except the N heteroatom attached to the pyrimidine ring;
R_{S1} is independently hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S1A})₂, —OR_{S1A}, —SR_{S1A}, —S(═O)R_{S1B}, —S(═O)₂R_{S1B}, —C(═O)R_{S1D}, —C(═O)OR_{S1A}, —OC(═O)R_{S1B}, —C(═O)N(R_{S1A})₂, —N_{S1A}C(═O)R_{S1B}, —OC(═O)OR_{S1A}, —N_{S1A}C(═O)OR_{S1A}, —NR_{S1A}C(═S)O_{S1A}, —OC(═O)N(R_{S1A})₂, —NR_{S1A}C(═O)N(R_{S1A})₂, —S(═O)OR_{S1A}, —OS(═O)R_{S1B}, —S(═O)N(R_{S1A})₂, —NR_{S1A}S(═O)R_{S1B}, —S(═O)₂OR_{S1A}, —OS(═O)₂R_{S1B}, —S(═O)₂N(R_{S1A})₂, —NR_{S1A}S(═O)₂R_{S1B}, —OS(═O)₂OR_{S1A}, —NR_{S1A}S(═O)₂OR_{S1A}, —OS(═O)₂N(R_{S1A})₂, —NR_{S1A}S(═O)₂N(R_{S1A})₂, —P(R_{S1A})₂, —P(═O)(R_{S1B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S1C})₂, —OR_{S1C}, —SR_{S1C}, —S(═O)R_{S1D}, —S(═O)₂R_{S1D}, —C(═O)R_{S1D}, —C(═O)OR_{S1C}, —OC(═O)R_{S1D}, —C(═O)N(R_{S1C})₂, —NR_{S1C}C(═O)R_{S1D}, —OC(═O)OR_{S1C}, —NR_{S1C}C(═O)OR_{S1C}, —NR_{S1C}C(═S)OR_{S1C}, —OC(═O)N(R_{S1C})₂, —NR_{S1C}C(═O)N(R_{S1C})₂, —S(═O)OR_{S1C}, —OS(═O)R_{S1D}, —S(═O)N(R_{S1C})₂, —NR_{S1C}S(═O)R_{S1D}, —S(═O)₂OR_{S1C}, —OS(═O)₂R_{S1D}, —S(═O)₂N(R_{S1C})₂, —NR_{S1C}S(═O)₂R_{S1D}, —OS(═O)₂OR_{S1C}, —NR_{S1C}S(═O)₂OR_{S1C}, —OS(═O)₂N(R_{S1C})₂, —NR_{S1C}S(═O)₂N(R_{S1C})₂, —P(R_{S1C})₂, —P(═O)(R_{S1D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, two R_{S1} together with the carbon atom to which they are both attached form

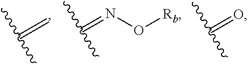

a 3-10 membered carbocyclic ring or a 3-10 heterocyclic ring; wherein, said

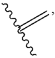

3-10 membered carbocyclic ring or 3-10 heterocyclic ring is independently unsubstituted or substituted with one or more R_{S11}:
Optionally, two adjacent R_{S1} together with the atoms to which they are respectively attached form

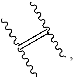 [SMILES: *=*]

a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S12};
Optionally, two nonadjacent R_{S1} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S13};
m₁ is 0, 1, 2, 3, 4, 5, 6, 7, 8 or 9;
R_{S2} is independently hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S2A})₂, —OR_{S2A}, —SR_{S2A}, —S(═O)R_{S2B}, —S(═O)₂R_{S2B}, —C(═O)R_{S2B}, —C(═O)OR_{S2A}, —OC(═O)R_{S2B}, —C(═O)N(R_{S2A})₂, —NR_{S2A}C(═O)R_{S2B}, —OC(═O)OR_{S2A}, —NR_{S2A}C(═O)OR_{S2A}, —NR_{S2A}C(═S)OR_{S2A}, —OC(═O)N(R_{S2A})₂, —NR_{S2A}C(═O)N(R_{S2A})₂, —S(═O)OR_{S2A}, —OS(═O)R_{S2B}, —S(═O)N(R_{S2A})₂, —NR_{S2A}S(═O)R_{S2B}, —S(═O)₂OR_{S2A}, —OS(═O)₂R_{S2B}, —S(═O)₂N(R_{S2A})₂, —NR_{S2A}S(═O)₂R_{S2B}, —OS(═O)₂OR_{S2A}, —NR_{S2A}S(═O)₂OR_{S2A}, —OS(═O)₂N(R_{S2A})₂, —NR_{S2A}S(═O)₂N(R_{S2A})₂, —P(R_{S2A})₂, —P(═O)(R_{S2B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S2C})₂, —OR_{S2C}, —SR_{S2C}, —S(═O)R_{S2D}, —S(═O)₂R_{S2D}, —C(═O)R_{S2D}, —C(═O)OR_{S2D}, —OC(═O)R_{S2D}, —C(═O)N(R_{S2C})₂, —NR_{S2C}C(═O)R_{S2D}, —OC(═O)OR_{S2C}, —NR_{S2C}C(═O)OR_{S2C}, —NR_{S2C}C(═S)OR_{S2C}, —OC(═O)N(R_{S2C})₂, —NR_{S2C}C(═O)N(R_{S2C})₂, —S(═O)OR_{S2C}, —OS(═O)R_{S2D}, —S(═O)N(R_{S2C})₂, —NR_{S2C}S(═O)R_{S2D}, —S(═O)₂OR_{S2C}, —OS(═O)₂R_{S2D}, —S(═O)₂N(R_{S2C})₂, —NR_{S2C}S(═O)₂R_{S2D}, —OS(═O)₂OR_{S2C}, —NR_{S2C}S(═O)₂OR_{S2C}, —OS(═O)₂N(R_{S2C})₂, —NR_{S2C}S(═O)₂N(R_{S2C})₂, —P(R_{S2C})₂, —P(═O)(R_{S2D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, two R_{S2} together with the carbon atom to which they are both attached form

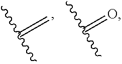

a 3-10 membered carbocyclic ring or a 3-10 heterocyclic ring; wherein, said

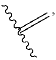

3-10 membered carbocyclic ring or 3-10 heterocyclic ring is independently unsubstituted or substituted with one or more R_{S21};
Optionally, two adjacent R_{S2} together with the atoms to which they are respectively attached form

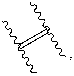 [SMILES: *=*]

a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S22};
Optionally, two nonadjacent R_{S2} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S23};
m₂ is 0, 1, 2, 3, 4 or 5;
Y, is a bond, O, S, S(═O), S(═O)₂ or NR_{Y11};
R_{Y11} is hydrogen, deuterium, —C₁₋₆alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
R₃ is

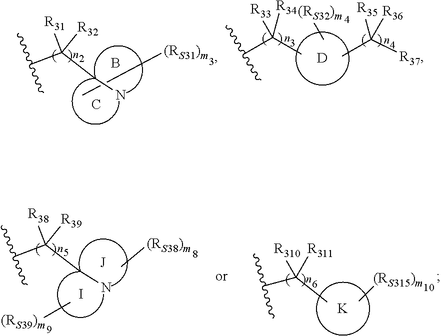

Each of R₃₁, R₃₂, R₃₃, R₃₄, R₃₅, R₃₆, R₃₈, R₃₉, R₃₁₀ and R₃₁₁ is independently hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, —S(═O)R_{B}, —S(═O)₂R_{A}, —C(═O)R_{A}, —C(═O)OR_{A}, —C(═O)N(R_{A})₂, —S(═O)OR_{A}, —S(═O)N(R_{A})₂, —S(═O)₂OR_{A}, —S(═O)₂N(R_{A})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NRCS(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, R₃₁ and R₃₂ together with the carbon atom to which they are both attached form,

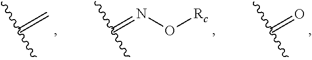

a 3-10 membered carbocyclic ring or a 3-10 heterocyclic ring; wherein, said

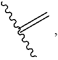

3-10 membered carbocyclic ring or 3-10 heterocyclic ring is independently unsubstituted or substituted with one or more R_{S33};
Optionally, R₃₃ and R₃₄ together with the carbon atom to which they are both attached form

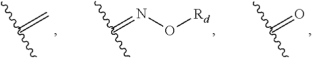

a 3-10 membered carbocyclic ring or a 3-10 heterocyclic ring; wherein, said

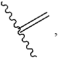

3-10 membered carbocyclic ring or 3-10 heterocyclic ring is independently unsubstituted or substituted with one or more R_{S34};
Optionally, R₃₅ and R₃₆ together with the carbon atom to which they are both attached form

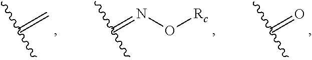

a 3-10 membered carbocyclic ring or a 3-10 heterocyclic ring; wherein, said

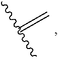

3-10 membered carbocyclic ring or 3-10 heterocyclic ring is independently unsubstituted or substituted with one or more R_{S35};
Optionally, R₃₈ and R₃₉ together with the carbon atom to which they are both attached form

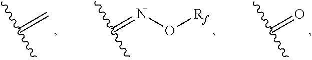

a 3-10 membered carbocyclic ring or a 3-10 heterocyclic ring; wherein, said

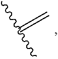

3-10 membered carbocyclic ring or 3-10 heterocyclic ring is independently unsubstituted or substituted with one or more R_{S310};
Optionally, R₃₁₀ and R₃₁₁ together with the carbon atom to which they are both attached form

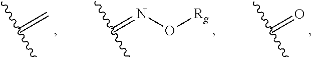

a 3-10 membered carbocyclic ring or a 3-10 heterocyclic ring; wherein, said

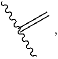

3-10 membered carbocyclic ring or 3-10 heterocyclic ring is independently unsubstituted or substituted with one or more R_{S316};
n₂ is 0, 1, 2, 3, 4, 5 or 6;
n₃ is 0, 1, 2, 3, 4, 5 or 6;
n₄ is 0, 1, 2, 3, 4, 5 or 6;
n₅ is 0, 1, 2, 3, 4, 5 or 6;
n₆ is 0, 1, 2, 3, 4, 5 or 6;
Ring B is a 3-10 membered heterocyclic ring optionally further containing 1, 2, or 3 heteroatoms selected from N, O, S, S(═O) or S(═O)₂;
Ring C is a 3-10 membered heterocyclic ring optionally further containing 1, 2, or 3 heteroatoms selected from N, O, S, S(═O) or S(═O)₂;
Ring D is a 3-10 membered carbocyclic ring or a 3-10 membered heterocyclic ring;
Ring I is a 3-10 membered carbocyclic ring or a 3-10 membered heterocyclic ring containing 1, 2, or 3 heteroatoms selected from N, O, S, S(═O) or S(═O)₂;
Ring J is a 3-10 membered carbocyclic ring or a 3-10 membered heterocyclic ring containing 1, 2, or 3 heteroatoms selected from N, O, S, S(═O) or S(═O)₂;
Ring K is 3-10 membered carbocyclic ring or a 3-10 membered heterocyclic ring containing 1, 2, or 3 heteroatoms selected from N, O, S, S(═O) or S(═O)₂;
R_{S31} is hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S31A})₂, —OR_{S31A}, —SR_{S31A}, —S(═O)R_{S31B}, —S(═O)₂R_{S31B}, —C(═O)R_{S31B}, —C(═O)OR_{S31A}, —OC(═O)R_{S31B}, —C(═O)N(R_{S31A})₂, —NR_{S31A}C(═O)R_{S31B}, —OC(═O)OR_{S31A}, —NR_{S31A}C(═O)OR_{S31A}, —NR_{S31A}C(═S)OR_{S31A}, —OC(═O)N(R_{S31A})₂, —NR_{S31A}C(═O)N(R_{S31A})₂, —S(═O)OR_{S31A}, —OS(═O)R_{S31B}, —S(═O)N(R_{S31A})₂, —NR_{S31A}S(═O)R_{S31B}, —S(═O)₂OR_{S31A}, —OS(═O)₂R_{S31B}, —S(═O)₂N(R_{S31A})₂, —NR_{S31A}S(═O)₂R_{S31B}, —OS(═O)₂OR_{S31A}, —NR_{S31A}S(═O)₂OR_{S31A}, —OS(═O)₂N(R_{S31A})₂, —NR_{S31A}S(═O)₂N(R_{S31A})₂, —P(R_{S31A})₂, —P(═O)(R_{S31B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₄alkynyl, —CN, —NO₂, —N₃, oxo, —N(R_{S31C})₂, —OR_{S31C}, —SR_{S31C}, —S(═O)R_{S31D}, —S(═O)₂R_{S31D}, —C(═O)R_{S31D}, —C(═O)OR_{S31C}, —OC(═O)R_{S31D}, —C(═O)N(R_{S31C})₂, —NR_{S31C}C(═O)R_{S31D}, —OC(═O)OR_{S31A}, —NR_{S31C}C(═O)OR_{S31C}, —NR_{S31C}C(═S)OR_{S31C}, —OC(═O)N(R_{S31C})₂, —NR_{S31C}C(═O)N(R_{S31C})₂, —S(═O)OR_{S31C}, —OS(═O)R_{S31D}, —S(═O)N(R_{S31C})₂, —NR_{S31C}S(═O)R_{S31C}, —S(═O)₂OR_{S31C}, —OS(═O)₂R_{S31D}, —S(═O)₂N(R_{S31C})₂, —NR_{S31C}S(═O)₂R_{S31D}, —OS(═O)₂OR_{S31C}, —NR_{S31C}S(═O)₂OR_{S31C}, —OS(═O)₂N(R_{S31C})₂, —NR_{S31C}S(═O)₂N(R_{S31C})₂, —P(R_{S31C})₂, —P(═O)(R_{S31D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, two R_{S31} together with the carbon atom to which they are both attached form

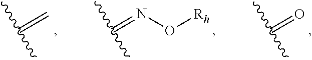

a 3-10 membered carbocyclic ring or a 3-10 membered heterocyclic ring; wherein, said

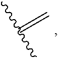

3-10 membered carbocyclic ring or 3-10 heterocyclic ring is independently unsubstituted or substituted with one or more R_{S311};
Optionally, two adjacent R_{S31} together with the carbon atoms to which they are respectively attached form

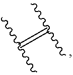 [SMILES: *=*]

a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S312};
Optionally, two nonadjacent R_{S31} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S313};
m₃ is 0, 1, 2, 3, 4, 5, 6, 7, 8, 9, 10, 11, or 12;
R_{S32} is hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S32A})₂, —OR_{S32A}, —SR_{S32A}, —S(═O)R_{S32B}, —S(═O)₂R_{S32B}, —C(═O)R_{S32B}, —C(═O)OR_{S32A}, —OC(═O)R_{S32B}, —C(═O)N(R_{S32A})₂, —NR_{S32A}C(═O)R_{S32B}, —OC(═O)OR_{S32A}, —NR_{S32A}C(═O)OR_{S32A}, —NR_{S32A}C(═S)OR_{S32A}, —OC(═O)N(R_{S32A})₂, —NR_{S32A}C(═O)N(R_{S32A})₂, —S(═O)OR_{S32A}, —OS(═O)R_{S32B}, —S(═O)N(R_{S32A})₂, —NR_{S32A}S(═O)R_{S32B}, —S(═O)₂OR_{S32A}, —OS(═O)₂R_{S32B}, —S(═O)₂N(R_{S32A})₂, —NR_{S32A}S(═O)₂R_{S32B}, —OS(═O)₂OR_{S32A}, —NR_{S32A}S(═O)₂OR_{S32A}, —OS(═O)₂N(R_{S32A})₂, —NR_{S32A}S(═O)₂N(R_{S32A})₂, —P(R_{S32A})₂, —P(═O)(R_{S32B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S32C})₂, —OR_{S32C}, —SR_{S32C}, —S(═O)R_{S32C}, —S(═O)₂R_{S32D}, —C(═O)R_{S32D}, —C(═O)OR_{S32C}, —OC(═O)R_{S32D}, —C(═O)N(R_{S32C})₂, —NR_{S32C}C(═O)R_{S32D}, —OC(═O)OR_{S32C}, —NR_{S32C}C(═O)OR_{S32C}, —NR_{S32C}C(═S)OR_{S32C}, —OC(═O)N(R_{S32C})₂, —NR_{S32C}C(═O)N(R_{S32C})₂, —S(═O)OR_{S32C}, —OS(═O)R_{S32C}, —S(═O)N(R_{S32C})₂, —NR_{S32C}S(═O)R_{S32D}, —S(═O)₂OR_{S32C}, —OS(═O)₂R_{S32D}, —S(═O)₂N(R_{S32C})₂, —NR_{S32C}S(═O)₂R_{S32D}, —OS(═O)₂OR_{S32C}, —NR_{S32C}S(═O))₂OR_{S32C}, —OS(═O)₂N(R_{S32C})₂, —NR_{S32C}S(═O)₂N(R_{S32C})₂, —P(R_{S32C})₂, —P(═O)(R_{S32D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, two R_{S32} together with the carbon atom to which they are both attached form

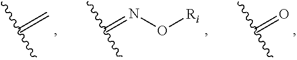

a 3-10 membered carbocyclic ring or a 3-10 heterocyclic ring; wherein, said

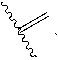

3-10 membered carbocyclic ring or 3-10 heterocyclic ring is independently unsubstituted or substituted with one or more R_{S321};
Optionally, two adjacent R_{S32} together with the carbon atoms to which they are respectively attached form

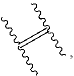 [SMILES: *=*]

a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S322};
Optionally, two nonadjacent R_{S32} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S323};
m₄ is 0, 1, 2, 3, 4, 5 or 6;
R₃₇ is —N(R_{37A})₂ or 3-10 membered heterocyclyl, wherein said 3-10 membered heterocyclyl is optionally independently substituted with one or more R_{S37};
R_{S38} is hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S38A})₂, —OR_{S38A}, —SR_{S38A}, —S(═O)R_{S38B}, —S(═O)₂R_{S38B}, —C(═O)R_{S38A}, —C(═O)OR_{S38A}, —OC(═O)R_{S38B}, —C(═O)N(R_{S38A})₂, —NR_{S38A}C(═O)R_{S38B}, —OC(═O)OR_{S38A}, —NR_{S38A}C(═O)OR_{S38A}, —NR_{S38A}C(═S)OR_{S38A}, —OC(═O)N(R_{S38A})₂, —NR_{S38A}C(═O)N(R_{S38A})₂, —S(═O)OR_{S38A}, —OS(═O)R_{S38B}, —S(═O)N(R_{S38A})₂, —NR_{S38A}S(═O)R_{S38B}, —S(═O)₂OR_{S38A}, —OS(═O)₂R_{S38B}, —S(═O)₂N(R_{S38A})₂, —NR_{S38A}S(═O)₂R_{S38B}, —OS(═O)₂OR_{S38A}, —NR_{S38A}S(═O)₂OR_{S38A}, —OS(═O)₂N(R_{S38A})₂, —NR_{S38A}S(═O)₂N(R_{S38A})₂, —P(R_{S38A})₂, —P(═O)(R_{S38B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S38C})₂, —OR_{S38C}, —SR_{S38C}, —S(═O)R_{S38D}, —S(═O))₂R_{S38D}, —C(═O)R_{S38D}, —C(═O)OR_{S38C}, —OC(═O)R_{S38D}, —C(═O)N(R_{S38C})₂, —NR_{S38C}C(═O)R_{S38D}, —OC(═O)OR_{S38C}, —NR_{S38C}C(═O)OR_{S38C}, —NR_{S38C}C(═S)OR_{S38C}, —OC(═O)N(R_{S38C})₂, —NR_{S38C}(═O)N(R_{S38C})₂, —S(═O)OR_{S38C}, —OS(═O)R_{S38D}, —S(═O)N(R_{S38C})₂, —NR_{S38C}S(═O)R_{S38D}, —S(═O)₂OR_{S38C}, —OS(═O))₂R_{S38D}, —S(═O)₂N(R_{S38C})₂, —NR_{S38C}S(═O)₂R_{S38D}, —OS(═O))₂OR_{S38C}, —NR_{S38C}S(═O)₂OR_{S38C}, —OS(═O)₂N(R_{S38C})₂, —NR_{S38C}S(═O))₂N(R_{S38C})₂, —P(R_{S38C})₂, —P(═O)(R_{S38D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, two R_{S38} together with the carbon atom to which they are both attached form

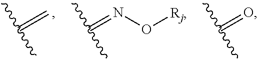

a 3-10 membered carbocyclic ring or a 3-10 membered heterocyclic ring; wherein, said

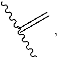

3-10 membered carbocyclic ring or 3-10 heterocyclic ring is independently unsubstituted or substituted with one or more R_{S381};
Optionally, two adjacent R_{S38} together with the carbon atoms to which they are respectively attached form

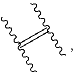 [SMILES: *=*]

a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S382};
Optionally, two nonadjacent R_{S38} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S383};
m₈ is 0, 1, 2, 3, 4, 5, 6, 7, 8, 9, 10, 11, or 12;
R_{S39} is hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S39A})₂, —OR_{S39A}, —SR_{S39A}, —S(═O)R_{S39B}, —S(═O)₂R_{S39B}, —C(═O)R_{S39B}, —C(═O)OR_{S39A}, —OC(═O)R_{S39B}, —C(═O)N(R_{S39A})₂, —NR_{S39A}C(═O)R_{S39B}, —OC(═O)OR_{S39A}, —NR_{S39A}C(═O)OR_{S39A}, —NR_{S39A}C(═S)OR_{S39A}, —OC(═O)N(R_{S39A})₂, —NR_{S39A}C(═O)N(R_{S39A})₂, —S(═O)OR_{S39A}, —OS(═O)R_{S39B}, —S(═O)N(R_{S39A})₂, —NR_{S39A}S(═O)R_{S39B}, —S(═O)₂OR_{S39A}, —OS(═O)₂R_{S39B}, —S(═O)₂N(R_{S39A})₂, —NR_{S39A}S(═O)₂R_{S39B}, —OS(═O)₂OR_{S39A}, —NR_{S39A}S(═O))₂OR_{S39A}, —OS(═O))₂N(R_{S39A})₂, —NR_{S39A}S(═O)₂N(R_{S39A})₂, —P(R_{S39A})₂, —P(═O)(R_{S39B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S39C})₂, —OR_{S39C}, —SR_{S39C}, —S(═O)R_{S39C}, —S(═O)₂R_{S39D}, —C(═O)R_{S39D}, —C(═O)OR_{S39C}, —OC(═O)R_{S39C}, —C(═O)N(R_{S39C})₂, —NR_{S39C}C(═O)R_{S39C}, —OC(═O)OR_{S39C}, —NR_{S39C}C(═O)OR_{S39C}, —NR_{S39C}C(═S)OR_{S39C}, —OC(═O)N(R_{S39C})₂, —NR_{S39C}C(═O)N(R_{S39C})₂, —S(═O)OR_{S39C}, —OS(═O)R_{S39C}, —S(═O)N(R_{S39C})₂—NR_{S39C}S(═O)R_{S39D}, —S(═O)₂OR_{S39C}, —OS(═O)₂R_{S39D}, —S(═O)₂N(R_{S39C})₂, —NR_{S39C}S(═O)₂R_{S39D}, —OS(═O)₂OR_{S39C}, —NR_{S39C}S(═O))₂OR_{S39C}, —OS(═O)₂N(R_{S39C})₂, —NR_{S39C}S(═O)₂N(R_{S39C})₂, —P(R_{S39C})₂, —P(═O)(R_{S39D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, two R_{S39} together with the carbon atom to which they are both attached form

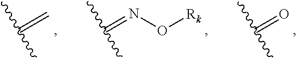

a 3-10 membered carbocyclic ring or a 3-10 heterocyclic ring; wherein, said

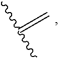

3-10 membered carbocyclic ring or 3-10 heterocyclic ring is independently unsubstituted or substituted with one or more R_{S391};
Optionally, two adjacent R_{S39} together with the carbon atoms to which they are respectively attached form

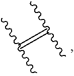 [SMILES: *=*]

a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S392};
Optionally, two nonadjacent R_{S39} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S393};
m₉ is 0, 1, 2, 3, 4, 5 or 6;
R_{S315} is hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S315A})₂, —OR_{S315A}, —SR_{S38A}, —S(═O)R_{S315B}, —S(═O)₂R_{S315B}, —C(═O)R_{S315B}, —C(═O)OR_{S315A}, —OC(═O)R_{S315B}, —C(═O)N(R_{S315A})₂, —NR_{S315A}C(═O)R_{S315B}, —OC(═O)OR_{S315A}, —NR_{S315A}C(═O)R_{S315A}, —NR_{S315A}C(═S)OR_{S315A}, —OC(═O)N(R_{S315A})₂, —NR_{S315A}C(═O)N(R_{S315A})₂, —S(═O)OR_{S315A}, —OS(═O)R_{S315B}, —S(═O)N(R_{S315A})₂, —NR_{S315A}S(═O)R_{S315B}, —S(═O)₂OR_{S315A}, —OS(═O)₂R_{S315B}, —S(═O)₂N(R_{S315A})₂, —NR_{S315A}S(═O)₂R_{S315B}, —OS(═O)₂OR_{S315A}, —NR_{S315A}S(═O)₂OR_{S315A}, —OS(═O)₂N(R_{S38A})₂, —NR_{S315A}S(═O)₂N(R_{S315A})₂, —P(R_{S315A})₂, —P(═O)(R_{S315B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S315C})₂, —OR_{S315C}, —SR_{S315C}, —S(═O)R_{S315C}, —S(═O)₂R_{S315D}, —C(═O)R_{S315D}, —C(═O)OR_{S315C}, —OC(═O)R_{S315D}, —C(═O)N(R_{S315C})₂, —NR_{S315D}C(═O)R_{S315D}, —OC(═O)OR_{S315C}, —NR_{S315C}C(═O)OR_{S315C}, —NR_{S315C}C(═S)OR_{S315C}, —OC(═O)N(R_{S315C})₂, —NR_{S315C}C(═O)N(R_{S315C})₂, —S(═O)OR_{S315C}, —OS(═O)R_{S315C}, —S(═O)N(R_{S315C})₂, —NR_{S315C}S(═O)R_{S315D}, —S(═O)₂OR_{S315C}, —OS(═O)₂R_{S315D}, —S(═O)₂N(R_{S315C})₂, —NR_{S315C}S(═O)₂R_{S315D}, —OS(═O)₂OR_{S315C}, —NR_{S315C}S(═O)₂OR_{S315C}, —OS(═O)₂N(R_{S315C})₂, —NR_{S315C}S(═O)₂N(R_{S315C})₂, —P(R_{S315C})₂, —P(═O)(R_{S315D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, two R_{S315} together with the carbon atom to which they are both attached form

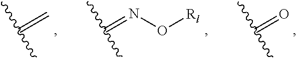

a 3-10 membered carbocyclic ring or a 3-10 heterocyclic ring; wherein, said

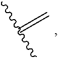

3-10 membered carbocyclic ring or 3-10 heterocyclic ring is independently unsubstituted or substituted with one or more R_{S3151};
Optionally, two adjacent R_{S315} together with the carbon atoms to which they are respectively attached form

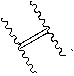 [SMILES: *=*]

a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S3152};
Optionally, two nonadjacent R_{S315} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S3153};
m₁₀ is 0, 1, 2, 3, 4, 5 or 6;
R₄ is 6-10 membered aryl, 5-10 membered heteroaryl,

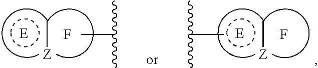

wherein said 6-10 membered aryl, 5-10 membered heteroaryl,

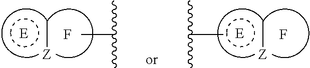

is independently unsubstituted or substituted with one or more R_{S4};
Z at each occurrence is independently C or N;
Ring E at each occurrence is independently a 6 membered aryl ring or a 5-6 membered heteroaryl ring and ring F at each occurrence is a 3-10 membered carbocyclic ring or a 3-10 membered heterocyclic ring when Z is C;
Ring E at each occurrence is a 5-6 membered heteroaryl ring and ring F at each occurrence is a 3-10 membered heterocyclic ring when Z is N;
R_{S4} is independently deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S4A})₂, —OR_{S4A}, —SR_{S4A}, —S(═O)R_{S4B}, —S(═O)₂R_{S4B}, —C(═O)R_{S4B}, —C(═O)OR_{S4A}, —OC(═O)R_{S4B}, —C(═O)N(R_{S4A})₂, —NR_{S4A}C(═O)R_{S4B}, —OC(═O)OR_{S4A}, —NR_{S4A}C(═O)OR_{S4A}, —NR_{S4A}C(═S)OR_{S4A}, —OC(═O)N(R_{S4A})₂, —NR_{S4A}C(═O)N(R_{S4A})₂, —S(═O)OR_{S4A}, —OS(═O)R_{S4B}, —S(═O)N(R_{S4A})₂, —NR_{S4A}S(═O)R_{S4B}, —S(═O)₂OR_{S4A}, —OS(═O)₂R_{S4B}, —S(═O)₂N(R_{S4A})₂, —NR_{S4A}S(═O)₂R_{S4B}, —OS(═O)₂OR_{S4A}, —NR_{S4A}S(═O)OR_{S4A}, —OS(═O)₂N(R_{S4A})₂, —NR_{S4A}S(═O)₂N(R_{S4A})₂, —P(R_{S4A})₂, —P(═O)(R_{S4B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S4C})₂, —OR_{S4C}, —SR_{S4C}, —S(═O)R_{S4D}, —S(═O)₂R_{S4D}, —C(═O)R_{S4D}, —C(═O)OR_{S4D}, —OC(═O)R_{S4D}, —C(═O)N(R_{S4C})₂, —NR_{S4C}C(═O)R_{S4D}, —OC(═O)OR_{S4C}, —NR_{S4C}C(═O)OR_{S4C}, —NR_{S4C}C(═S)OR_{S4C}, —OC(═O)N(R_{S4C})₂, —NR_{S4C}C(═O))N(R_{S4C})₂, —S(═O))OR_{S4C}, —OS(═O))R_{S4D}, —S(═O)N(R_{S4C})₂, —NR_{S4C}S(═O) R_{S4D}, —S(═O))₂OR_{S4C}, —OS(═O))₂R_{S4D}, —S(═O))₂N(R_{S4C})₂, —NR_{S4C}S(═O)₂R_{S4D}, —OS(═O)₂OR_{S4C}, —NR_{S4C}S(═O)₂OR_{S4C}, —OS(═O)₂N(R_{S4C})₂, —NR_{S4C}S(═O)₂N(R_{S4C})₂, —P(R_{S4C})₂, —P(═O)(R_{S4D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
R₅ is hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{5A})₂, —OR_{5A}, —SR_{5A}, —S(═O)R_{5B}, —S(═O)₂R_{5B}, —C(═O)R_{5B}, —C(═O)OR_{5A}, —OC(═O)R_{5B}, —C(═O)N(R_{5A})₂, —NR_{5A}C(═O)R_{5B}, —OC(═O)OR_{5A}, —NR_{5A}C(═O)OR_{5A}, —NR_{5A}C(═S)OR_{5A}, —OC(═O)N(R_{5A})₂, —NR_{5A}C(═O)N(R_{5A})₂, —S(═O)OR_{5A}, —OS(═O)R_{5B}, —S(═O)N(R_{5A})₂, —NR_{5A}S(═O)R_{5B}, —S(═O)₂OR_{5A}, —OS(═O)₂R_{5B}, —S(═O)₂N(R_{5A})₂, —NR_{5A}S(═O)₂R_{5B}, —OS(═O)₂OR_{5A}, —NR_{5A}S(═O)₂OR_{5A}, —OS(═O)₂N(R_{5A})₂, —NR_{5A}S(═O)₂N(R_{5A})₂, —P(R_{5A})₂, —P(═O)(R_{5B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{5C})₂, —OR_{5C}, —SR_{5C}, —S(═O)R_{5D}S(═O)₂R_{5D}, —C(═O)R_{5D}, —C(═O)OR_{5D}, —OC(═O)R_{D}, —C(═O)N(R_{5C})₂, —NR_{5C}C(═O)R_{5D}, —OC(═O)OR_{5C}, —NR_{5C}C(═O)OR_{5C}, —NR_{5C}C(═S)OR_{5C}, —OC(═O)N(R_{5C})₂—NR_{C}C(═O)N(R_{C})₂, —S(═O)OR_{5C}, —OS(═O)R_{5D}, —S(═O)N(R_{5C})₂, —NR_{5C}S(═O)R_{5D}, —S(═O)₂OR_{5C}, —OS(═O)₂R_{5D}, —S(═O)₂N(R_{5C})₂, —NR_{5C}S(═O)₂R_{5D}, —OS(═O)₂OR_{5C}, —NR_{5C}S(═O)₂OR_{5C}, —OS(═O)₂N(R_{5C})₂, —NR_{5C}S(═O)₂N(R_{5C})₂, —P(R_{5C})₂, —P(═O)(R_{5D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Each of R_{1A}, R_{C}, R_{S1A}, R_{S1C}, R_{S2A}, R_{S2C}, R_{S31A}, R_{S31C}, R_{S32A}, R_{S32C}, R_{37A}, R_{S38A}, R_{S38C}, R_{S39A}, R_{S39C}, R_{S315A}, R_{S315C}, R_{S4A}, R_{S4C}, R_{5A}, R_{5C}, Rₐ, R_{b}, R_{c}, R_{d}, Rₑ, R_{f}, R_{g}, Rₕ, Rᵢ, Rⱼ, Rₖ and Rₗ is independently hydrogen, deuterium, —C₁₋₆alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═S)N(R_{C})₂, —NR_{C}C(═O)N(R_{C})₂, —S(═O)OR_{C}, —OS(═O)R_{D}, —S(═O)N(R_{C})₂, —NR_{C}S(═O)R, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Optionally, (two R_{1A}, two R_{1C}, two R_{S1A}, two R_{S1C}, two R_{S2A}, two R_{S2C}, two R_{S31A}, two R_{S31C}, two R_{S32A}, two R_{S32C}, two R_{37A}, two R_{S38A}, two R_{S38C}, two R_{S39A}, two R_{S39C}, two R_{S315A}, two R_{S315C}, two R_{S4A}, two R_{S4C}, two R_{5A} or two R_{5C}) together with the nitrogen atom to which they are both attached form a 3-10 membered heterocyclic ring or a 5-10 membered heteroaryl ring, wherein, said 3-10 membered heterocyclic ring or 5-10 membered heteroaryl ring is independently unsubstituted or substituted with one or more R_{SS};
Each of R_{1B}, R_{1D}, R_{S1B}, R_{S1D}, R_{S2B}, R_{S2D}, R_{S31B}, R_{S31D}, R_{S32B}, R_{S32D}, R_{S38B}, R_{S38D}, R_{S39B}, R_{S315B}, R_{S315D}, R_{S39D}, R_{S4B}, R_{S4D}, R_{5B} and R_{5D} is independently hydrogen, deuterium, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NRCS(═O)₂R_{D}, —OS(═O)₂OR_{C}, —NRCS(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Each of (R_{A}, R_{B}, R_{C} and R_{D}) is independently hydrogen, deuterium, —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more R_{SA};
Each of R_{SX1}, R_{S11}, R_{S12}, R_{S13}, R_{S21}, R_{S22}, R_{S23}, R_{S33}, R_{S34}, R_{S35}, R_{S37}, R_{S310}, R_{S316}, R_{S311}, R_{S312}, R_{S313}, R_{S321}, R_{S322}, R_{S323}, R_{S381}, R_{S382}, R_{S383}, R_{S391}, R_{S392}, R_{S393}, R_{S3151}, R_{S3152}, R_{S3153}, R_{SS} and R_{SA} is independently deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —O(C₁₋₆alkyl), —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₄alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein, said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₃alkyl, haloC₁₋₃alkyl, haloC₁₋₃alkoxy, —C₂₋₃alkenyl, —C₂₋₃alkynyl, —CN, —NO₂, —N₃, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —OC(═O)O(C₁₋₃alkyl), —NHC(═O)(OC₁₋₃alkyl), —N(C₁₋₃alkyl)C(═O)(OC₁₋₃alkyl), —OC(═O)NH(C₁₋₃alkyl), —OC(═O)N(C₁₋₃alkyl)₂, —NHC(═O)NH₂, —NHC(═O)NH(C₁₋₃alkyl), —NHC(═O)N(C₁₋₃alkyl)₂, —N(C₁₋₃alkyl)C(═O)NH₂, —N(C₁₋₃alkyl)C(═O)NH(C₁₋₃alkyl), —N(C₁₋₃alkyl)C(═O)N(C₁₋₃alkyl)₂, —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —OS(═O)₂O(C₁₋₃alkyl), —NHS(═O)₂O(C₁₋₃alkyl), —N(C₁₋₃alkyl)S(═O)₂O(C₁₋₃alkyl), —OS(═O)₂NH₂, —OS(═O)₂NH(C₁₋₃alkyl), —OS(═O)₂N(C₁₋₃alkyl)₂, —NHS(═O)₂NH₂, —NHS(═O)₂NH(C₁₋₃alkyl), —NHS(═O)₂N(C₁₋₃alkyl)₂, —N(C₁₋₃alkyl)S(═O)₂NH₂, —N(C₁₋₃alkyl)S(═O)₂NH(C₁₋₃alkyl), —N(C₁₋₃alkyl)S(═O)₂N(C₁₋₃alkyl)₂, —PH(C₁₋₃alkyl), —P(C₁₋₃alkyl)₂, —P(═O)H(C₁₋₃alkyl), —P(═O)(C₁₋₃alkyl)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Each of heterocyclyl or heterocyclic at each occurrence independently contains 1, 2, 3 or 4 heteroatoms selected from N, O, S. S(═O) or S(═O)₂;
Each of heteroaryl at each occurrence independently contains 1, 2, 3 or 4 heteroatoms selected from N, O, or S.

[2]. The compound of [1], wherein, the compound is any one of following formulas:

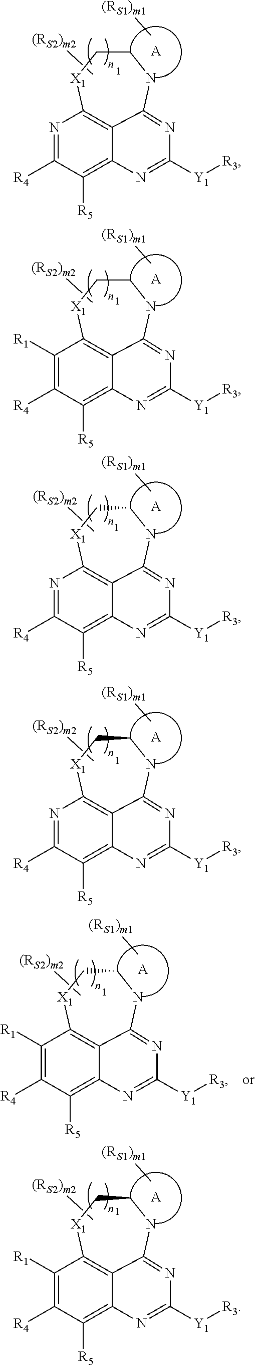

[3]. The compound of [1] or [2], wherein, R₁ is hydrogen, deuterium, halogen, —CN, —OC₁₋₆alkyl, —C₁₋₆alkyl, haloC₁₋₆alkyl, haloC₁₋₆alkoxy, —C₂₋₆alkenyl, —C₂₋₆alkynyl, or 3-6 membered cycloalkyl; said —OC₁₋₆alkyl, —C₁₋₆alkyl, —C₂₋₆alkenyl, —C₂₋₆alkynyl, or 3-6 membered cycloalkyl is unsubstituted or substituted with 1, 2 or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl.

In some embodiments, R₁ is

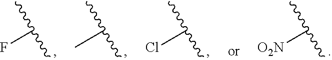

In some embodiments, R, is hydrogen, deuterium, —F, —Cl, —Br, —CN, —OCH₃, —CF₃, —CH₂CH₂CN, methyl, ethyl, or cyclopropyl.

In some embodiments, R₁ is

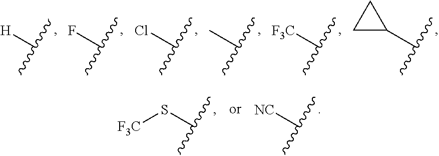

In some embodiments, R₁ is —NO₂.

In some embodiments, R₁ is —Cl,

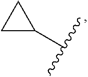 [SMILES: *C1CC1]

### —H, or —F.

[4]. The compound of any one of [1] to [3], wherein, R, is hydrogen, deuterium, —F, —Cl, —Br, —CN, —OCH₃, —CF₃, —CH₂CH₂CN, methyl, ethyl, or cyclopropyl.

[5]. The compound of any one of [1] to [4], wherein:
X₁ at each occurrence is independently —C(R_{X11})(R_{X12})—, —NR_{X13}—, —O—, —S— or —S(═O)—;
R_{X11} or R_{X12} is independently hydrogen, deuterium, halogen, —C₁₋₆alkyl or 3-6 membered cycloalkyl; wherein said —C₁₋₆alkyl or 3-6 membered cycloalkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl;
Optional R_{X11} and R_{X12} together with the carbon atom to which they are both attached form

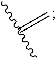

wherein, said

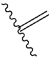

is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl;
R_{X13} is hydrogen, deuterium, —C₁₋₆alkyl or 3-6 membered cycloalkyl; wherein said —C₁₋₆alkyl or 3-6 membered cycloalkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl.

In some embodiments, R_{X13} is —CH₃, —CD₃,

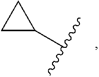 [SMILES: *C1CC1 ; ","]

—CH₂CH₃, —C(═O)CH₃ or —CH₂CH₂CF₃.

[6]. The compound of any one of [1] to [5], wherein:
R_{X11} or R_{X12} is independently hydrogen, deuterium, —F, methyl, —CD₃, ethyl, propyl, isopropyl or cyclopropyl;
Optionally, R_{X11} and R_{X12} together with the carbon atom to which they are both attached form

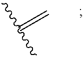

R_{X13} is independently hydrogen, deuterium, methyl, —CD₃, ethyl, propyl, isopropyl or cyclopropyl.

[7]. The compound of any one of [1] to [6], wherein, X₁ is —CH₂—, —CD₂-,

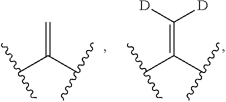

—O—, —S—, —S(═O)—, —NH—, —N(CH₃)— or —N(CD₃)-.

In some embodiments, X₁ is —CH₂—. In some embodiments, X₁ is —CD₂-. In some embodiments. X₁ is

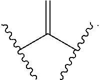 [SMILES: *C(*)=C]

In some embodiments. X₁ is

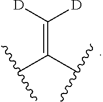 [SMILES: *C(*)=C([2H])[2H]]

In some embodiments, X₁ is —O—. In some embodiments, X₁ is —S—. In some embodiments, X₁ is —S(═O)—. In some embodiments, X₁ is —NH— or —N(CH₃)—. In some embodiments, X₁ is —O— or —N(CH₃)—. In some embodiments, X₁ is —O— or —N(CD₃)-. In some embodiments, X₁ is —N(CH₃)—. In some embodiments. X₁ is —N(CD₃)-. In some embodiments, X₁ is —S(═O)₂—.

[8]. The compound of any one of [1] to [7], wherein, n₁ is 0, 1, 2 or 3. In some embodiments, n₁ is 0, 1, or 2. In some embodiments, n₁ is 0 or 1. In some embodiments, n₁ is 0. In some embodiments, n₁ is 1. In some embodiments, n₁ is 2. In some embodiments, n₁ is 3. In some embodiments, X₁ is —O—, n₁ is 0. In some embodiments, X₁ is —O—, n₁ is 1. In some embodiments, X₁ is —O—, n₁ is 2. In some embodiments, X₁ is —O—, n₁ is 3. In some embodiments. X₁ is —NCH₃—, n₁ is 0. In some embodiments, X₁ is —NCH₃—, n₁ is 1. In some embodiments, X₁ is —NCH₃—, n₁ is 2. In some embodiments, X₁ is —NCH₃—, n₁ is 3. In some embodiments, X₁ is —CH₂—, n₁ is 0. In some embodiments. X₁ is —CH₂—, n₁ is 1. In some embodiments, X₁ is —CH₂—, n₁ is 2. In some embodiments, X₁ is —CH₂—, n₁ is 3. In some embodiments, X₁ is —CD₂-, n₁ is 0. In some embodiments. X₁ is —CD₂-, n₁ is 1. In some embodiments, X₁ is —CD₂-, n₁ is 2. In some embodiments, X₁ is —CD₂-, n₁ is 3. In some embodiments, X₁ is

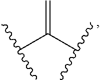 [SMILES: *C(*)=C]

n₁ is 0. In some embodiments, X₁ is

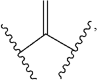 [SMILES: *C(*)=C]

n₁ is 1. In some embodiments, X₁ is

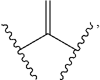 [SMILES: *C(*)=C]

n₁ is 2. In some embodiments, X₁ is

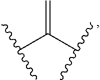 [SMILES: *C(*)=C]

n₁ is 3. In some embodiments, X₁ is

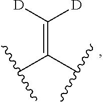 [SMILES: *C(*)=C([2H])[2H]]

n₁ is 0. In some embodiments, X₁ is

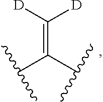 [SMILES: *C(*)=C([2H])[2H]]

n₁ is 1. In some embodiments, X₁ is

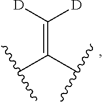 [SMILES: *C(*)=C([2H])[2H]]

n₁ is 2. In some embodiments, X₁ is

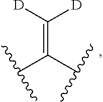 [SMILES: *C(*)=C([2H])[2H]]

n₁ is 3. In some embodiments, X₁ is —NCD₃-, n₁ is 0. In some embodiments, X₁ is —NCD₃-, n₁ is 1. In some embodiments, X₁ is —NCD₃-, n₁ is 2. In some embodiments, X₁ is —NCD₃-, n₁ is 3.

[9]. The compound of any one of [1] to [8], wherein, m₂ is 0, 1, 2, 3, 4, 5, or 6. In some embodiments, m₂ is 0, 1, 2, 3, 4 or 5. In some embodiments, m₂ is 0, 1, 2, 3 or 4. In some embodiments, m₂ is 0, 1, 2 or 3. In some embodiments, m₂ is 0, 1 or 2. In some embodiments, m₂ is 0 or 1. In some embodiments, m₂ is 0. In some embodiments, m₂ is 1. In some embodiments, m₂ is 2. In some embodiments, m₂ is 3. In some embodiments, m₂ is 4. In some embodiments, m₂ is 5. In some embodiments, m₂ is 6.

[10]. The compound of any one of [1] to [9], wherein, the compound is any one of the following formulas:

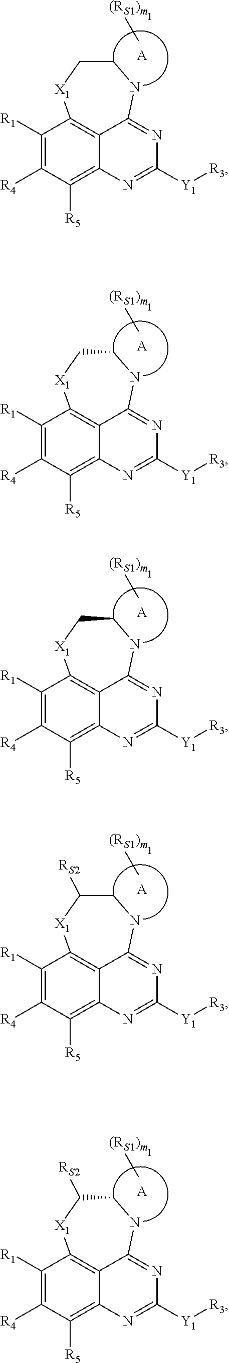

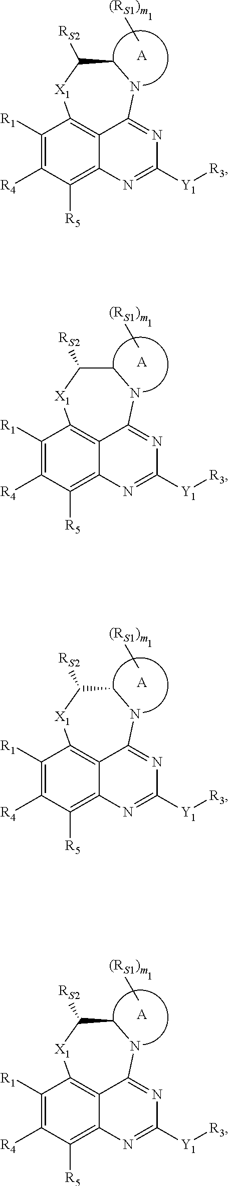

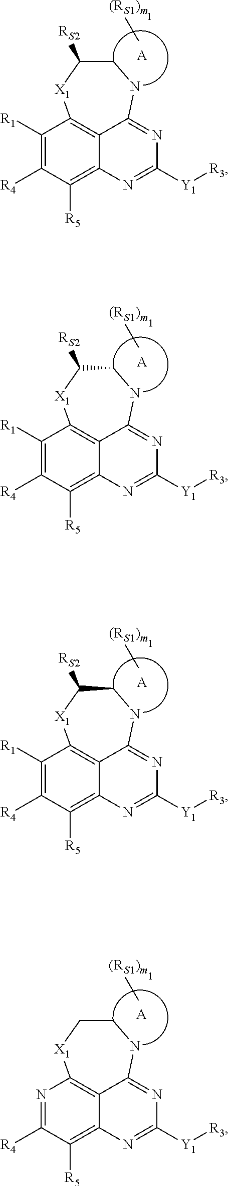

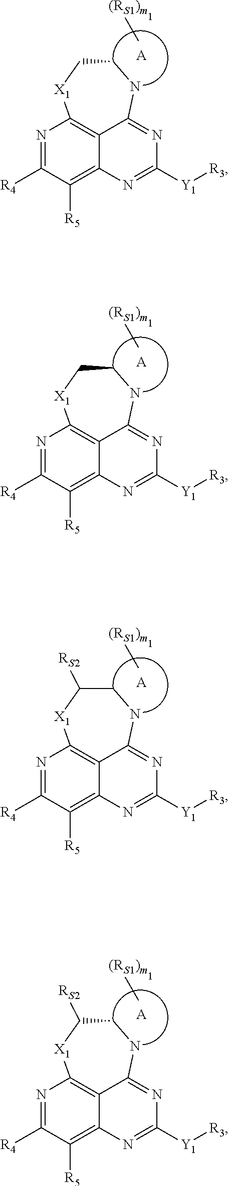

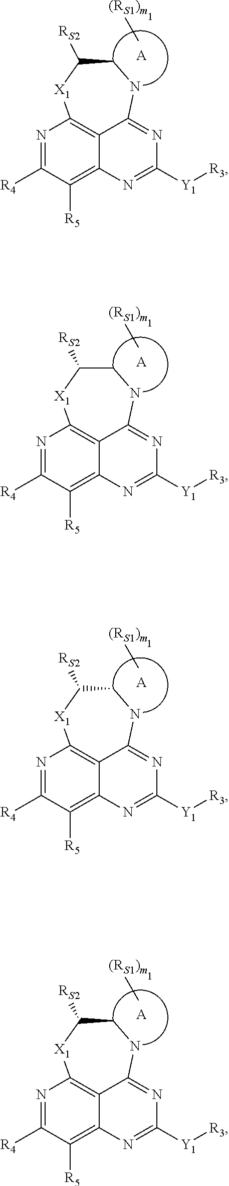

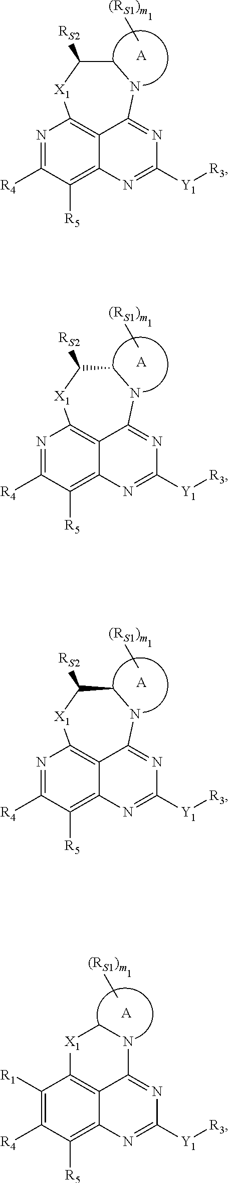

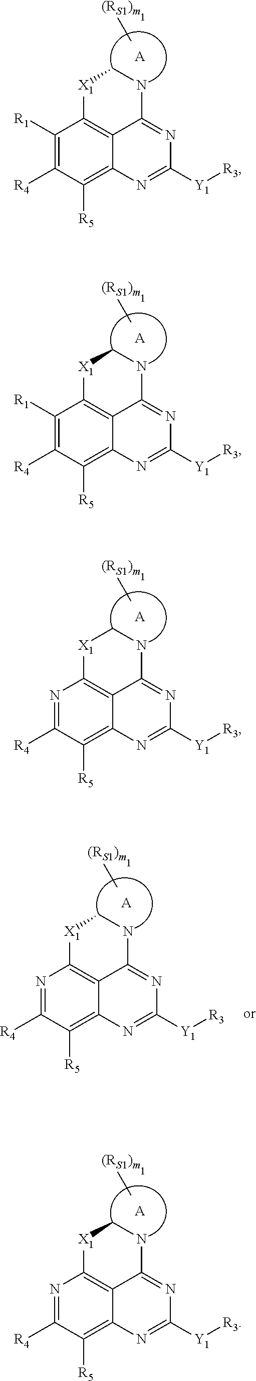

[11]. The compound of any one of [1] to [10], wherein, R_{S2} is deuterium, halogen or —C₁₋₆alkyl; wherein said —C₁₋₆alkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl;
Optionally, two adjacent R_{S2} together with the atoms to which they are respectively attached form

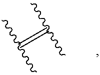 [SMILES: *=* ; ","]

a 3-6 membered carbocyclic ring, a 3-6 membered heterocyclic ring, a phenyl ring or a 5-6 membered heteroaryl ring, wherein, each of rings is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl.

[12]. The compound of any one of [1] to [11], wherein, R_{S2} is deuterium, —F, —CH₃ or —CD₃. In some embodiments, R_{S2} is —CH₃. In some embodiments, R_{S2} is deuterium.

[13]. The compound of any one of [1] to [12], wherein, m₁ is 0, 1, 2, 3, 4, 5, or 6. In some embodiments, m₁ is 0, 1, 2, 3 or 4. In some embodiments, m₁ is 0, 1, 2 or 3. In some embodiments, m₁ is 0, 1 or 2. In some embodiments, m₁ is 0 or 1. In some embodiments, m₁ is 0. In some embodiments, m₁ is 1. In some embodiments, m₁ is 2. In some embodiments, m₁ is 3. In some embodiments, m₁ is 4. In some embodiments, m₁ is 5. In some embodiments, m₁ is 6.

[14]. The compound of any one of [1] to [13], wherein:
Ring A is a 3-10 (such as 3, 4, 5, 6, 7, 8, 9 or 10) membered heterocyclic ring only comprising the N atom attached to the pyrimidine ring, or a 3-10 (such as 3, 4, 5, 6, 7, 8, 9 or 10) membered heterocyclic ring comprising one or more additional heteroatoms O, S, S═O or S(═O)₂ except the N heteroatom attached to the pyrimidine ring. In some embodiments, ring A is a 3-10 (such as 3, 4, 5, 6, 7, 8, 9 or 10) membered heterocyclic ring only comprising the N atom attached to the pyrimidine ring, or a 3-10 (such as 3, 4, 5, 6, 7, 8, 9 or 10) membered heterocyclic ring comprising one additional heteroatom O, S, S═O or S(═O)₂ except the N heteroatom attached to the pyrimidine ring. In some embodiments, Ring A is a 3-10 (such as 3, 4, 5, 6, 7, 8, 9 or 10) membered monocyclic heterocyclic ring only comprising the N atom attached to the pyrimidine ring, a 3-10 (such as 3, 4, 5, 6, 7, 8, 9 or 10) membered bicyclic heterocyclic ring only comprising the N atom attached to the pyrimidine ring, a 3-10 (such as 3, 4, 5, 6, 7, 8, 9 or 10) membered bridged heterocyclic ring only comprising the N atom attached to the pyrimidine ring, a 3-10 (such as 3, 4, 5, 6, 7, 8, 9 or 10) membered fused heterocyclic ring only comprising the N atom attached to the pyrimidine ring, a 3-10 (such as 3, 4, 5, 6, 7, 8, 9 or 10) membered spiro heterocyclic ring only comprising the N atom attached to the pyrimidine ring, a 3-10 (such as 3, 4, 5, 6, 7, 8, 9 or 10) membered monocyclic heterocyclic ring comprising one additional heteroatoms selected from O, S, S═O or S(═O)₂ except the N heteroatom attached to the pyrimidine ring, a 3-10 (such as 3, 4, 5, 6, 7, 8, 9 or 10) membered bicyclic heterocyclic ring comprising one additional heteroatoms selected from O, S, S═O or S(═O)₂ except the N heteroatom attached to the pyrimidine ring, a 3-10 (such as 3, 4, 5, 6, 7, 8, 9 or 10) membered bridged heterocyclic ring comprising one additional heteroatoms selected from O, S, S═O or S(═O)₂ except the N heteroatom attached to the pyrimidine ring, a 3-10 (such as 3, 4, 5, 6, 7, 8, 9 or 10) membered fused heterocyclic ring comprising one additional heteroatoms selected from O, S, S═O or S(═O)₂ except the N heteroatom attached to the pyrimidine ring, a 3-10 (such as 3, 4, 5, 6, 7, 8, 9 or 10) membered spiro heterocyclic ring comprising one additional heteroatoms selected from O, S, S═O or S(═O)₂ except the N heteroatom attached to the pyrimidine ring; wherein, each of rings is fully saturated or has one or more degrees (such as 1, 2 or 3) of unsaturation. In some embodiments, Ring A is a 5-10 (such as 5, 6, 7, 8, 9 or 10) membered monocyclic heterocyclic ring only comprising the N atom attached to the pyrimidine ring, a 5-10 (such as 5, 6, 7, 8, 9 or 10) membered bicyclic heterocyclic ring only comprising the N atom attached to the pyrimidine ring, a 5-10 (such as 5, 6, 7, 8, 9 or 10) membered bridged heterocyclic ring only comprising the N atom attached to the pyrimidine ring, a 5-10 (such as 5, 6, 7, 8, 9 or 10) membered fused heterocyclic ring only comprising the N atom attached to the pyrimidine ring, a 5-10 (such as 5, 6, 7, 8, 9 or 10) membered spiro heterocyclic ring only comprising the N atom attached to the pyrimidine ring, a 5-10 (such as 5, 6, 7, 8, 9 or 10) membered monocyclic heterocyclic ring comprising one additional heteroatom selected from O except the N heteroatom attached to the pyrimidine ring, a 5-10 (such as 5, 6, 7, 8, 9 or 10) membered bicyclic heterocyclic ring comprising one additional heteroatom selected from O except the N heteroatom attached to the pyrimidine ring, a 5-10 (such as 5, 6, 7, 8, 9 or 10) membered bridged heterocyclic ring comprising one additional heteroatom selected from O except the N heteroatom attached to the pyrimidine ring, a 5-10 (such as 5, 6, 7, 8, 9 or 10) membered fused heterocyclic ring comprising one additional heteroatom selected from O except the N heteroatom attached to the pyrimidine ring, a 5-10 (such as 5, 6, 7, 8, 9 or 10) membered spiro heterocyclic ring comprising one additional heteroatom selected from O except the N heteroatom attached to the pyrimidine ring; wherein, each of rings is fully saturated or has one degree of unsaturation. In some embodiments, Ring A is a 5-10 (such as 5, 6, 7, 8, 9 or 10) membered monocyclic heterocyclic ring only comprising the N atom attached to the pyrimidine ring, wherein, the ring is fully saturated or has one degree of unsaturation. In some embodiments, Ring A is a 5-10 (such as 5, 6, 7, 8, 9 or 10) membered bicyclic heterocyclic ring only comprising the N atom attached to the pyrimidine ring, wherein, the ring is fully saturated or has one degree of unsaturation. In some embodiments, Ring A is a 5-10 (such as 5, 6, 7, 8, 9 or 10) membered bridged heterocyclic ring only comprising the N atom attached to the pyrimidine ring, wherein, the ring is fully saturated or has one degree of unsaturation. In some embodiments, Ring A is a 5-10 (such as 5, 6, 7, 8, 9 or 10) membered fused heterocyclic ring only comprising the N atom attached to the pyrimidine ring, wherein, the ring is fully saturated or has one degree of unsaturation. In some embodiments, Ring A is a 5-10 (such as 5, 6, 7, 8, 9 or 10) membered spiro heterocyclic ring only comprising the N atom attached to the pyrimidine ring, wherein, the ring is fully saturated or has one degree of unsaturation. In some embodiments, Ring A is a 5-10 (such as 5, 6, 7, 8, 9 or 10) membered bridged heterocyclic ring only comprising the N atom attached to the pyrimidine ring, wherein, the ring is fully saturated or has one degree of unsaturation. In some embodiments, Ring A is a 5-10 (such as 5, 6, 7, 8, 9 or 10) membered monocyclic heterocyclic ring comprising one additional heteroatom selected from O except the N heteroatom attached to the pyrimidine ring, wherein, the ring is fully saturated or has one degree of unsaturation. In some embodiments, Ring A is a 5-10 (such as 5, 6, 7, 8, 9 or 10) membered bicyclic heterocyclic ring comprising one additional heteroatom selected from O except the N heteroatom attached to the pyrimidine ring, wherein, the ring is fully saturated or has one degree of unsaturation. In some embodiments, Ring A is a 5-10 (such as 5, 6, 7, 8, 9 or 10) membered bridged heterocyclic ring comprising one additional heteroatom selected from O except the N heteroatom attached to the pyrimidine ring, wherein, the ring is fully saturated or has one degree of unsaturation. In some embodiments, Ring A is a 5-10 (such as 5, 6, 7, 8, 9 or 10) membered fused heterocyclic ring comprising one additional heteroatom selected from O except the N heteroatom attached to the pyrimidine ring, wherein, the ring is fully saturated or has one degree of unsaturation. In some embodiments, Ring A is a 5-10 (such as 5, 6, 7, 8, 9 or 10) membered spiro heterocyclic ring comprising one additional heteroatom selected from O except the N heteroatom attached to the pyrimidine ring, wherein, the ring is fully saturated or has one degree of unsaturation.

[15]. The compound of any one of [1] to [14], wherein:
Ring A is:

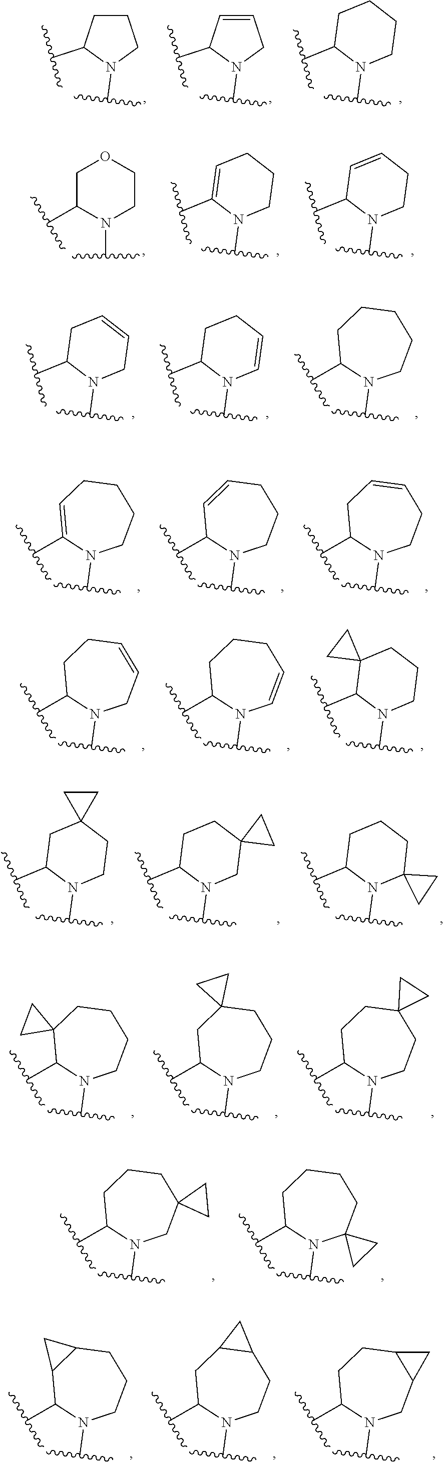

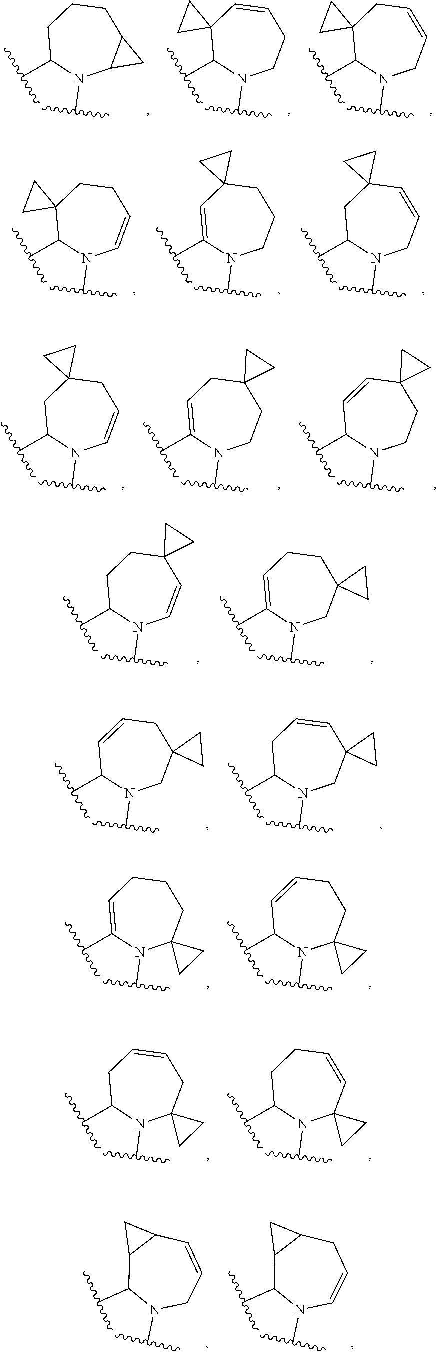

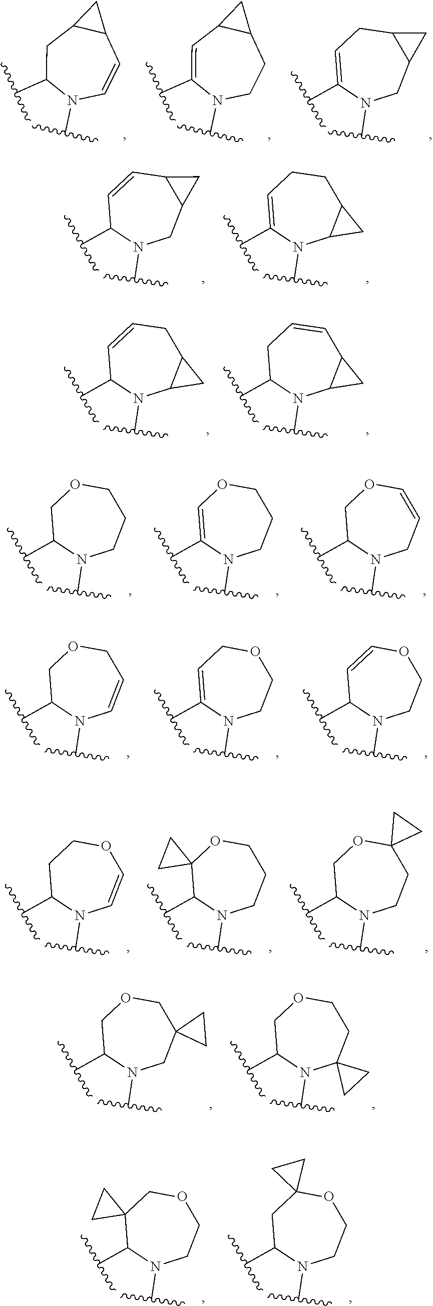

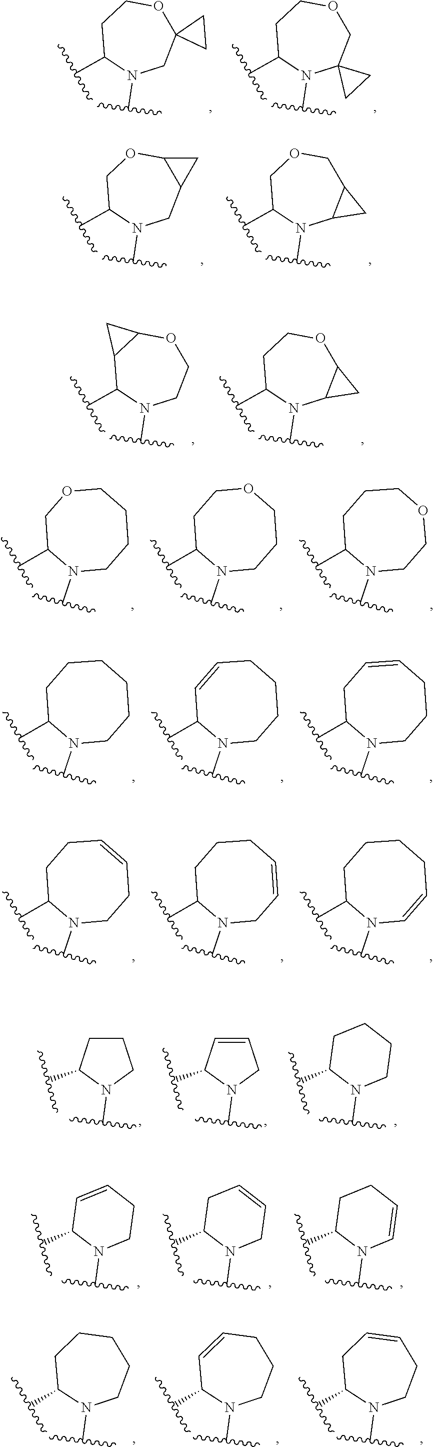

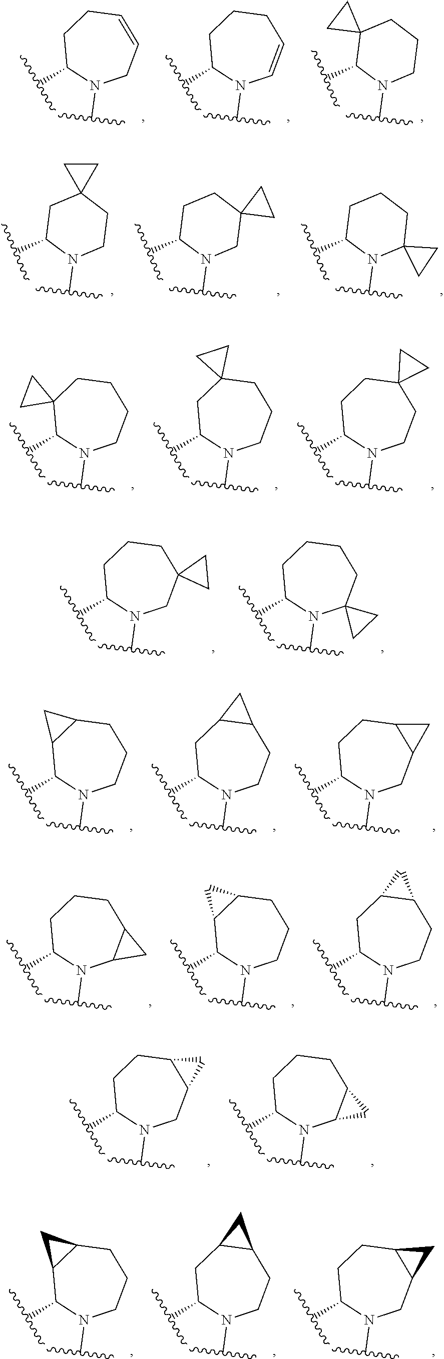

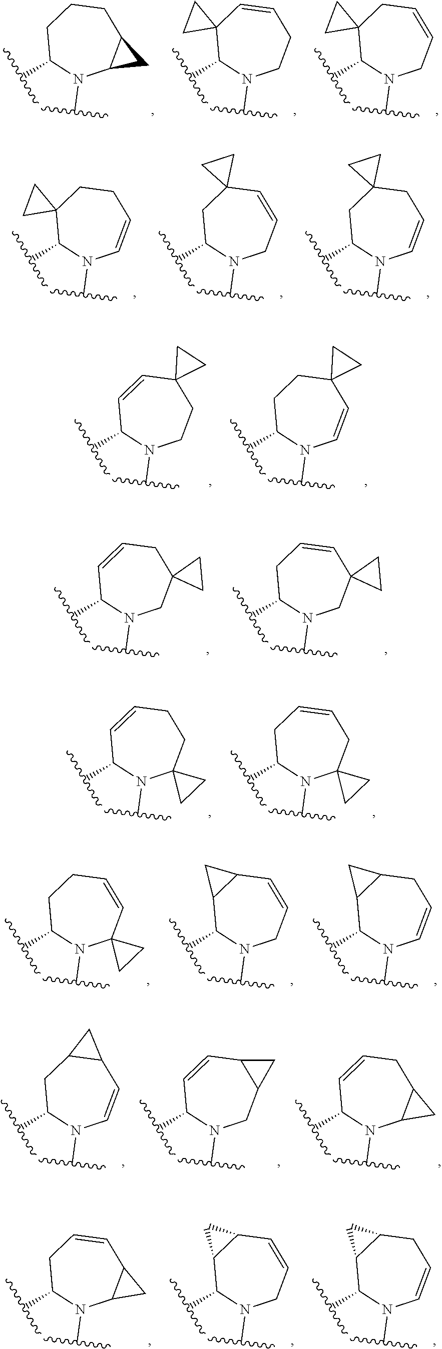

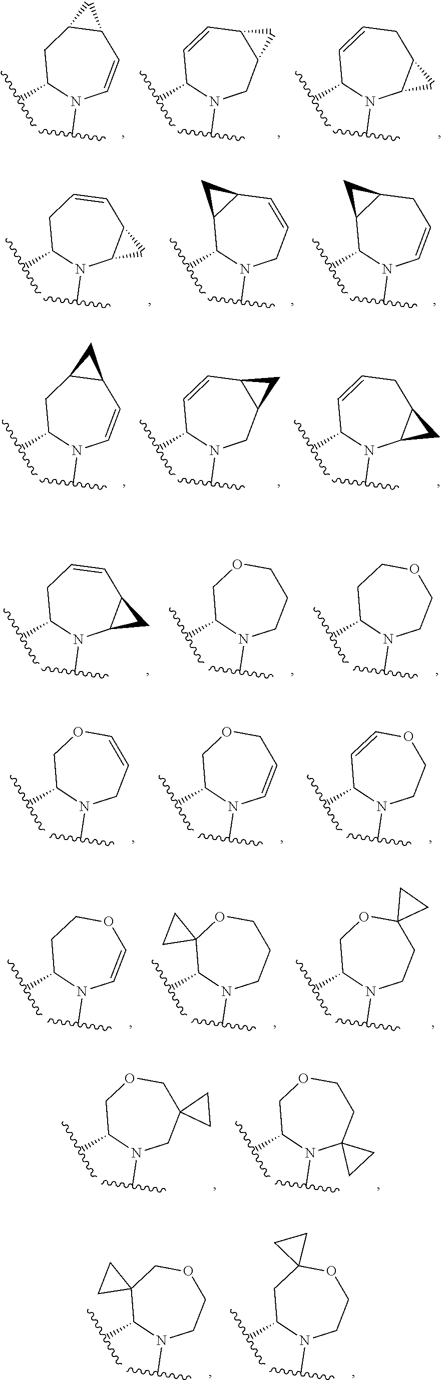

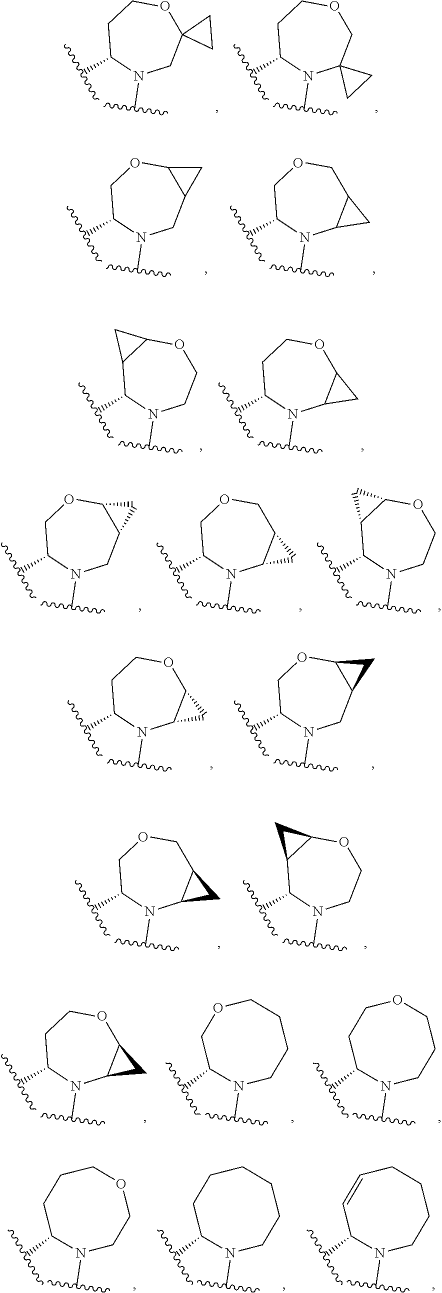

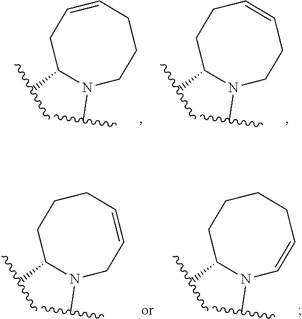 [SMILES: *[C@@H]1C/C=C\CCCN1* ; *[C@@H]1CC/C=C\CCN1* ; *[C@@H]1CCC/C=C\CN1* ; *[C@@H]1CCCC/C=C\N1*]

Each of ring A is optionally independently unsubstituted or substituted with m₁ R_{S1}.

[16]. The compound of any one of [1] to [15], wherein:
R_{S1} is deuterium, halogen, —C₁₋₆alkyl, —C₂₋₆alkenyl, —C₂₋₆alkynyl, haloC₁₋₆alkyl, haloC₁₋₆alkoxy, —CN, —NH₂, —NH(C₁₋₆alkyl), —N(C₁₋₆alkyl)₂, —OH, —OC₁₋₆alkyl or 3-6 membered cycloalkyl; wherein said —C₁₋₆alkyl, —C₂₋₆alkenyl, —C₂₋₆alkynyl or 3-6 membered cycloalkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆ alkyl;
Optionally, two R_{S1} together with the carbon atom to which the are both attached form

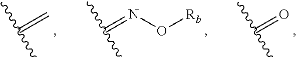

or a 3-6 membered carbocyclic ring; wherein, said

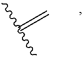

3-6 membered carbocyclic ring is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl;
Optionally, two adjacent R_{S1} together with the atoms to which they are respectively attached form

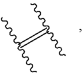 [SMILES: *=* ; ","]

a 3-6 membered carbocyclic ring; wherein, said 3-6 membered carbocyclic ring is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl.

In some embodiments, R_{S1} is —F, —OH, —OCH₃, —CN, —CH₂F, —CF₃, —CH₂OCH₃, —CH₂CN, —CHF₂, —CD₃, —NH₂ or —CH₃; or two R_{S1} together with the carbon atom to which they are both attached form

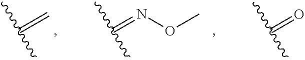

or a cyclopropyl ring; or two adjacent R_{S1} together with the atoms to which they are respectively attached form a cyclopropyl ring.

In some embodiments, R_{S1} is —F, —CH₃, —OH, —OCH₃, —CHF₂, —CH₂OCH₃,

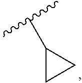 [SMILES: *C1CC1]

—Cl, —CH₂CH₃, -D,

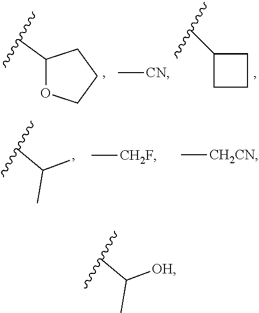

or —CH₂OH; or two R_{S1} together with the carbon atom to which they are both attached form

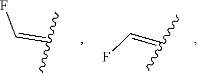

a cyclopropyl ring,

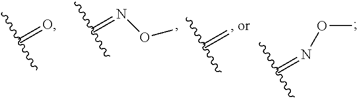

or two adjacent R_{S1} together with the atoms to which they are respectively attached form

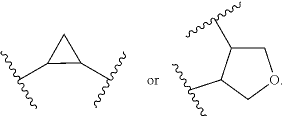 [SMILES: *C1CC1* ; *C1COCC1*]

In some embodiments,

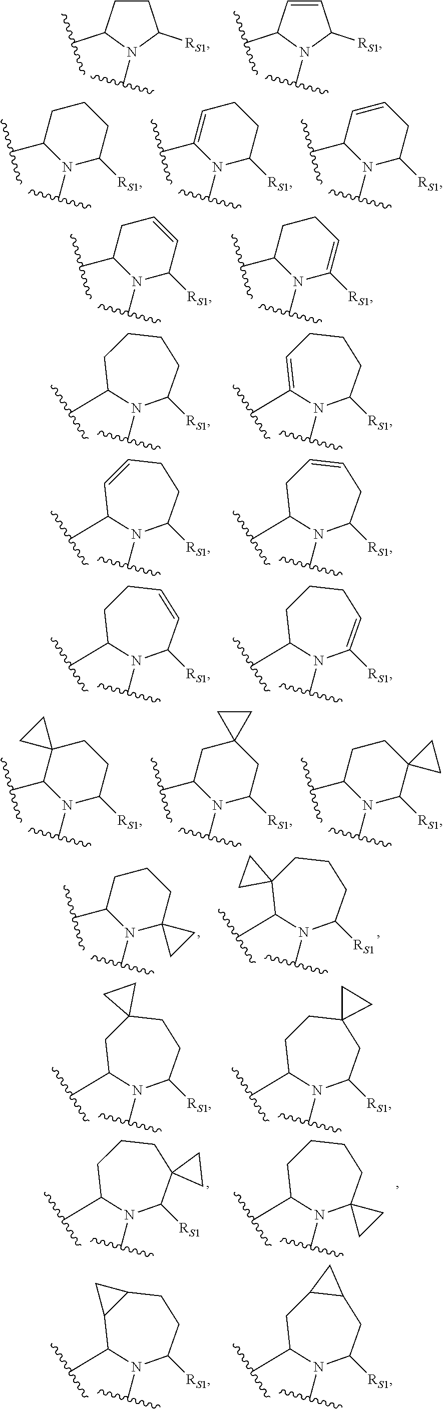

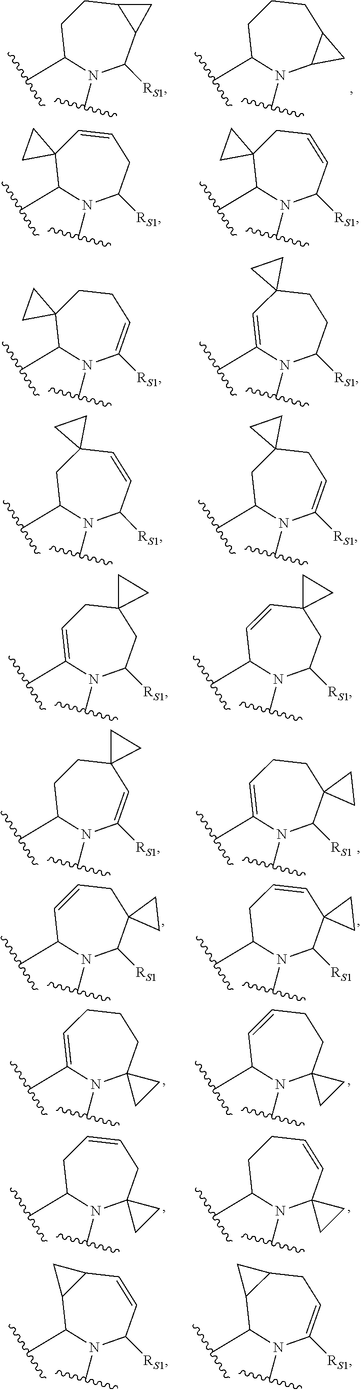

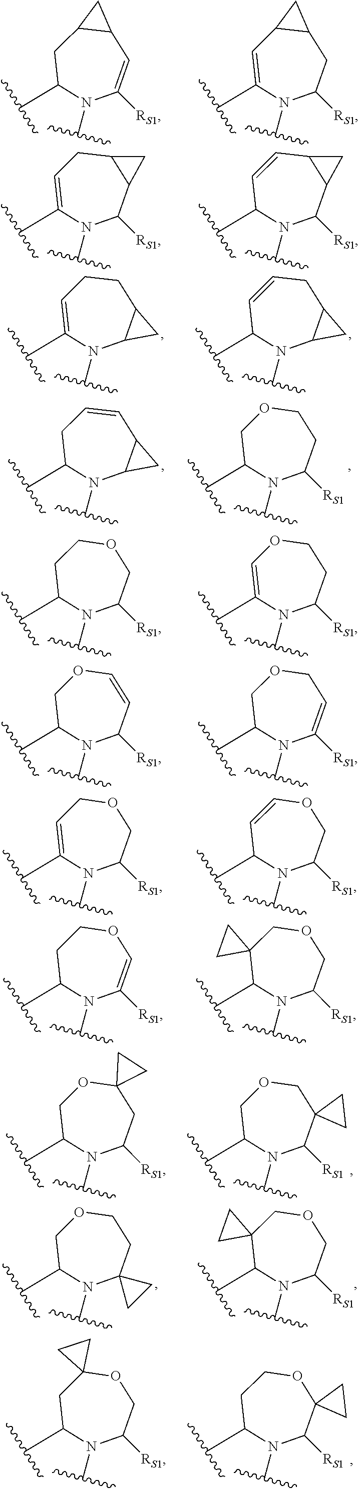

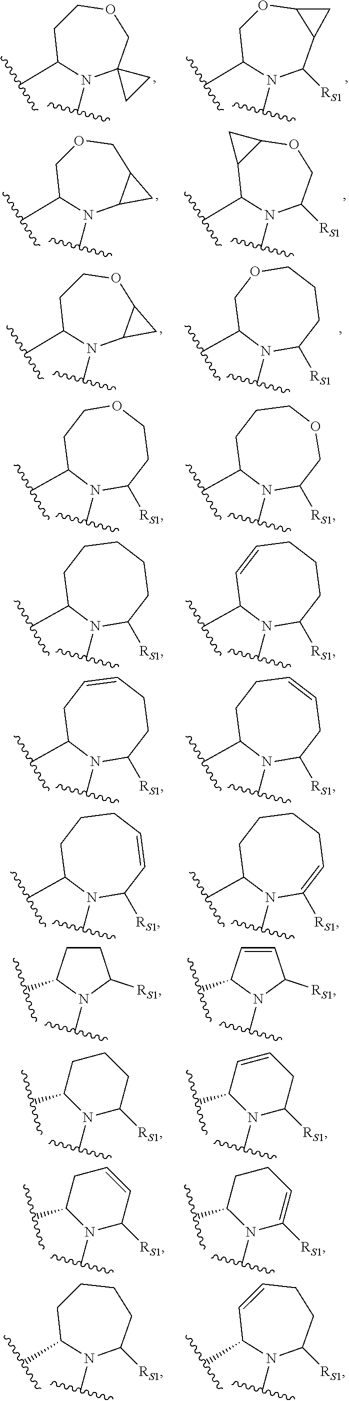

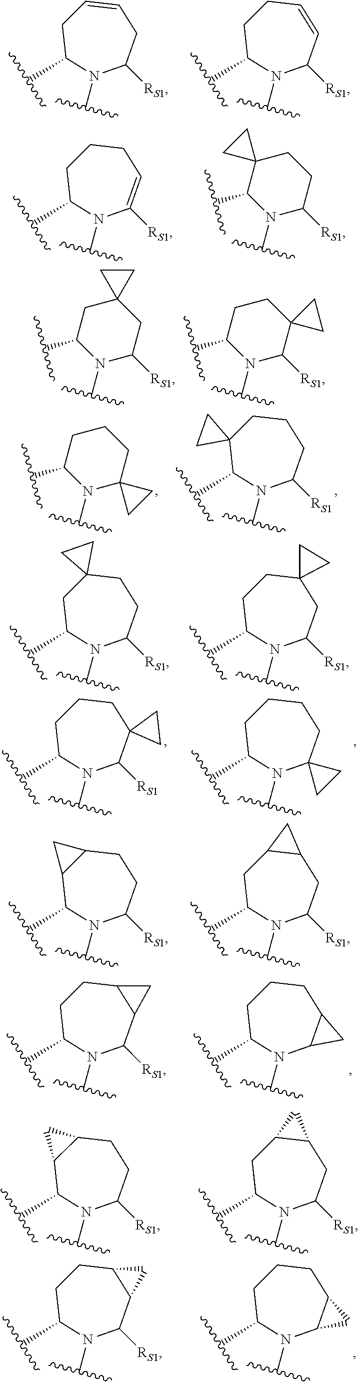

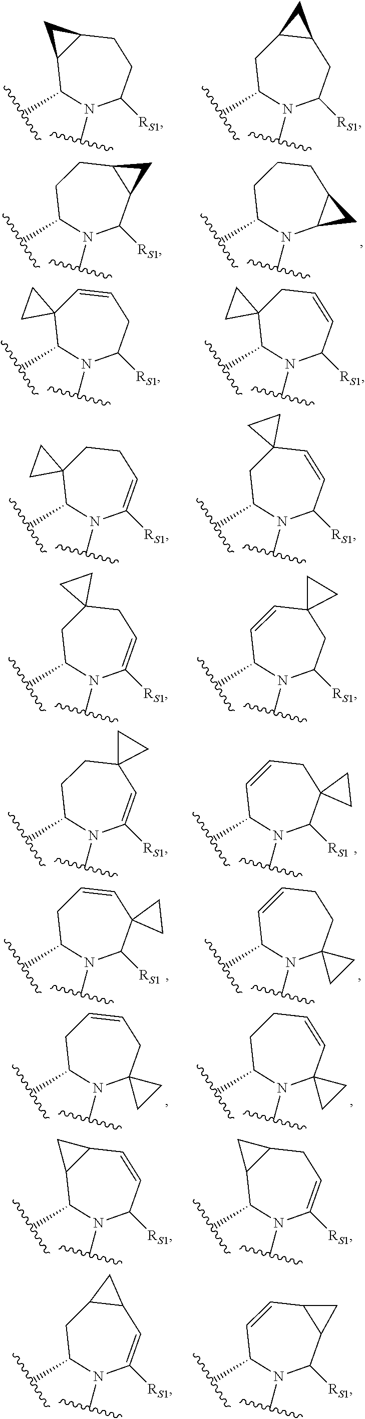

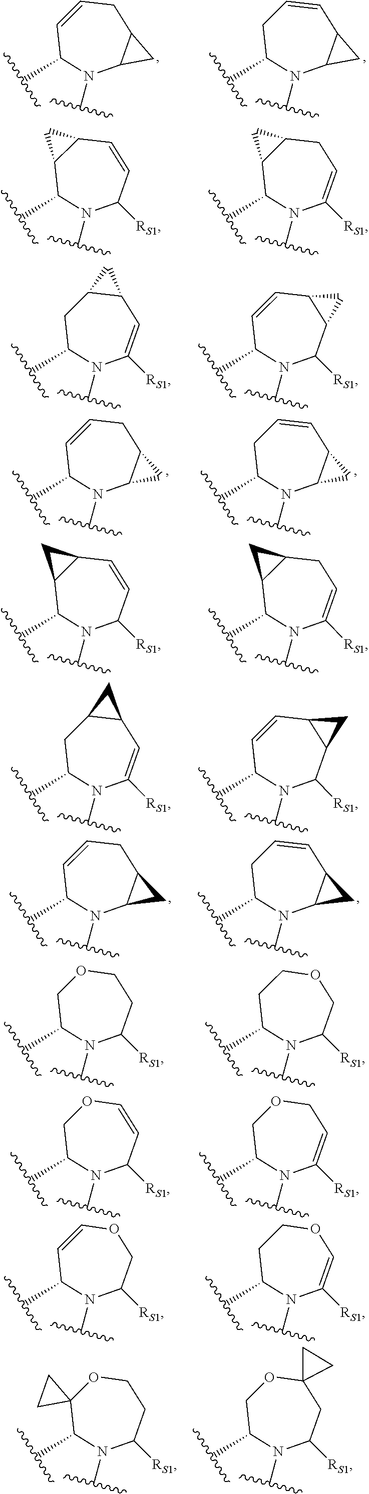

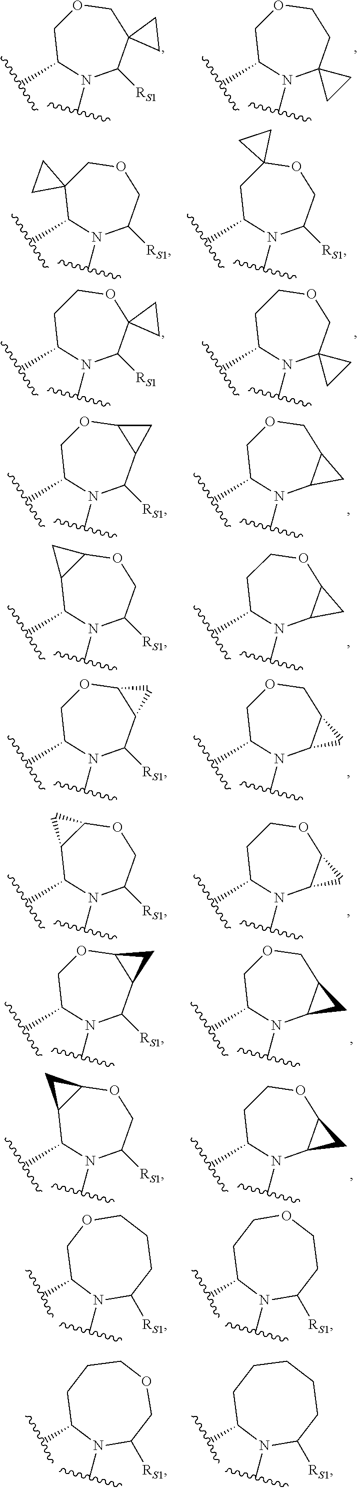

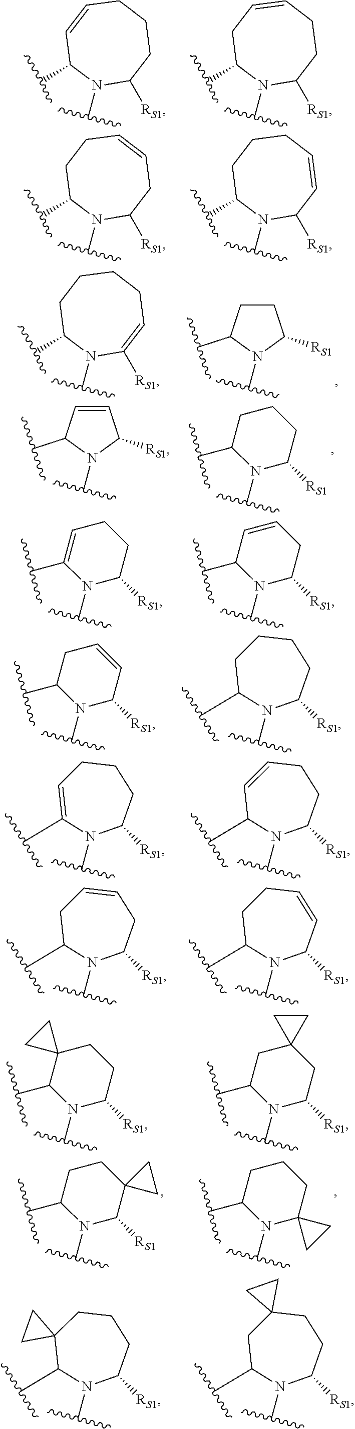

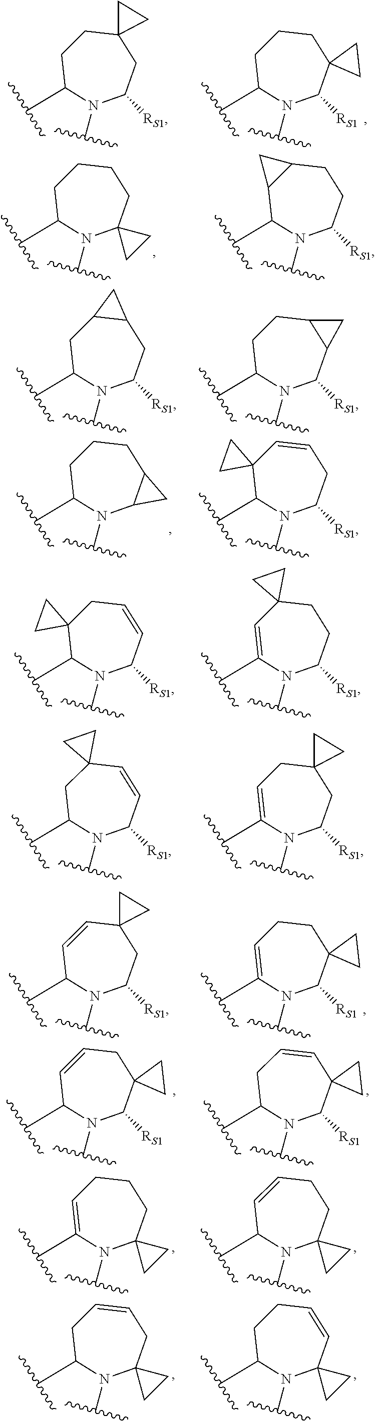

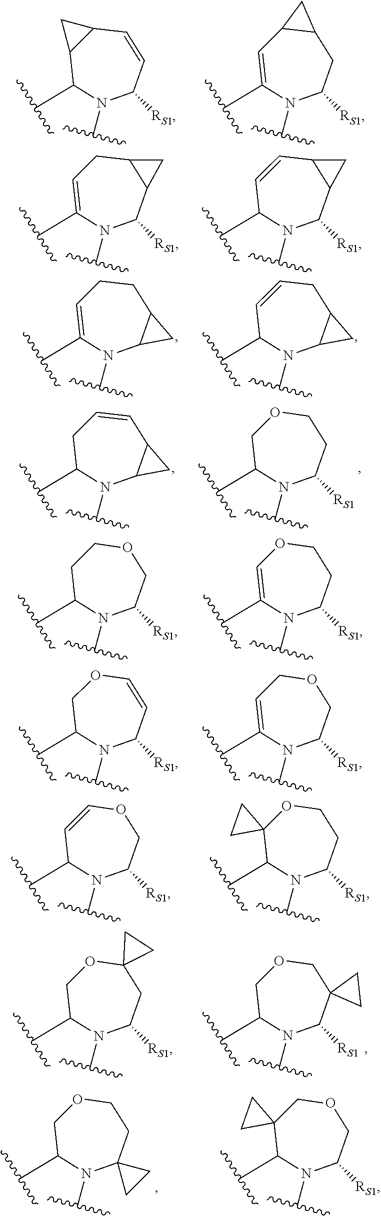

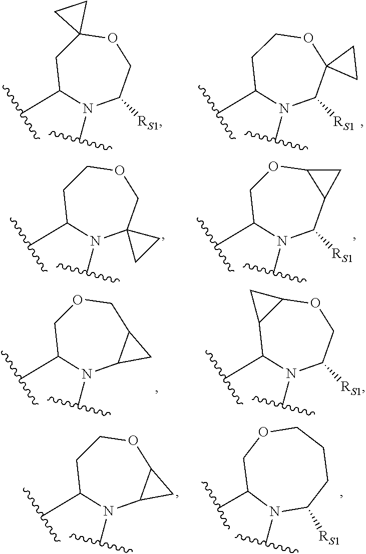

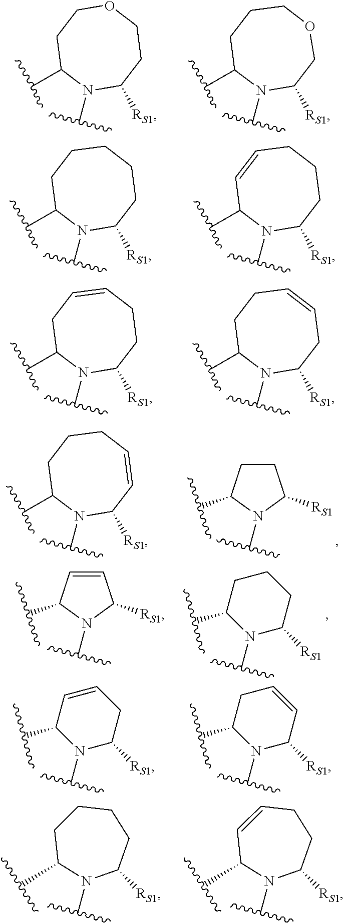

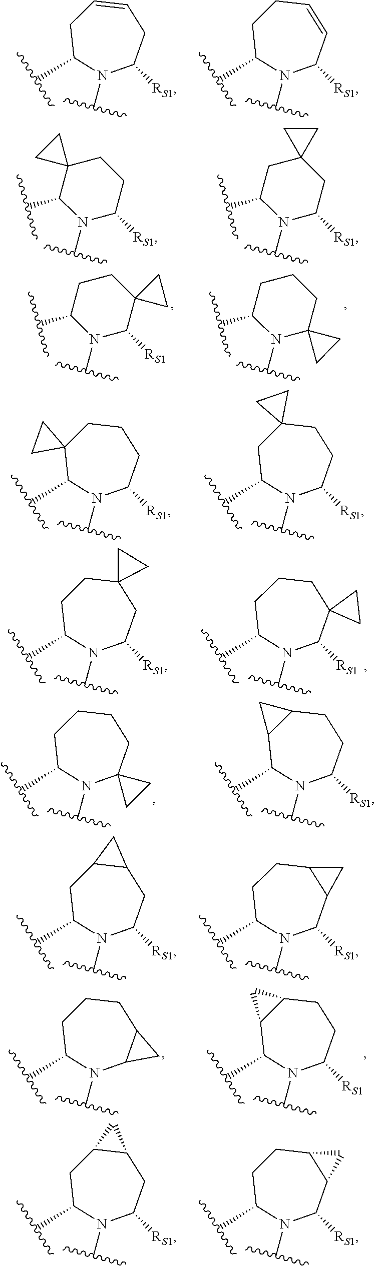

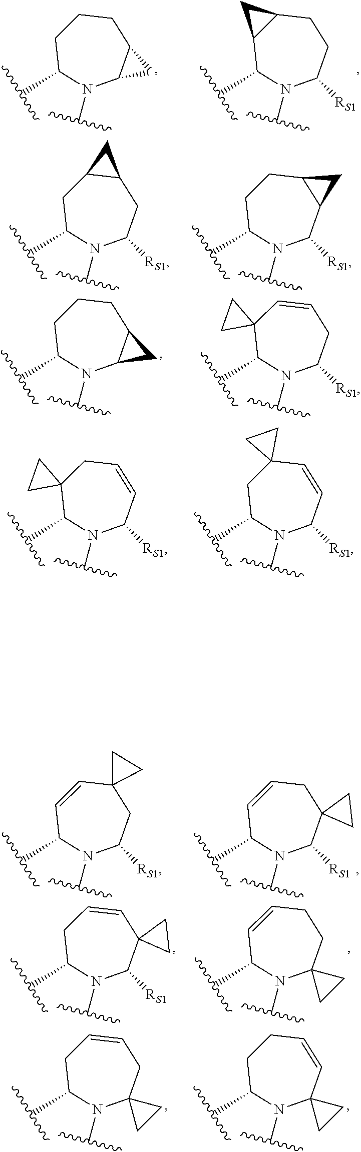

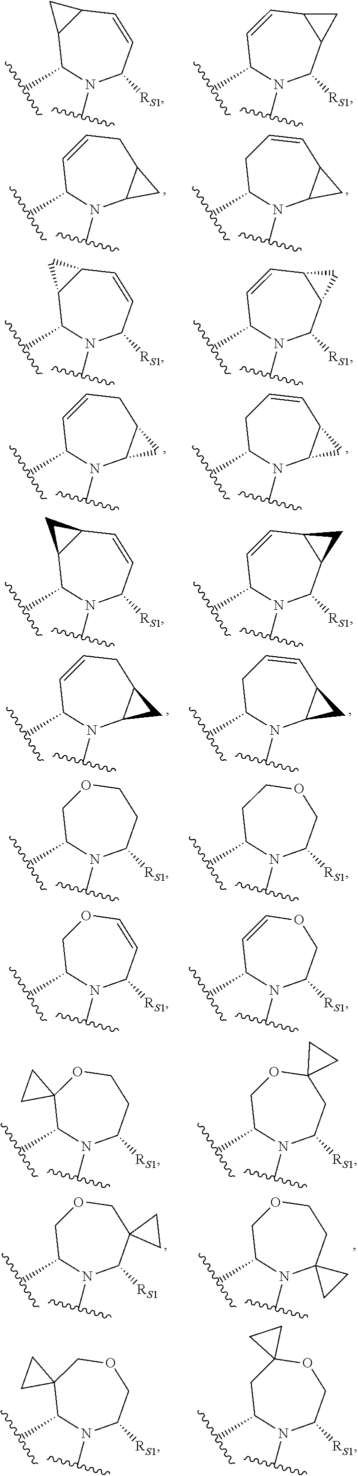

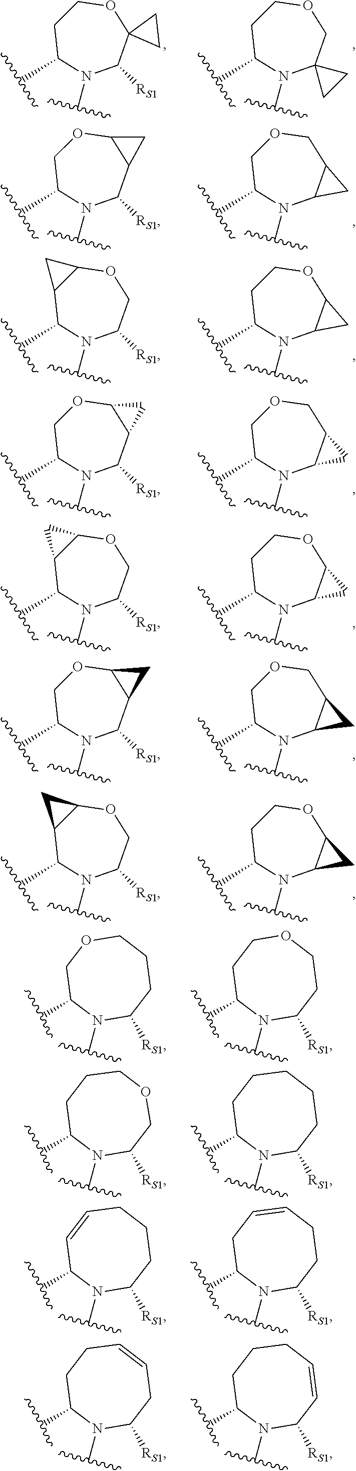

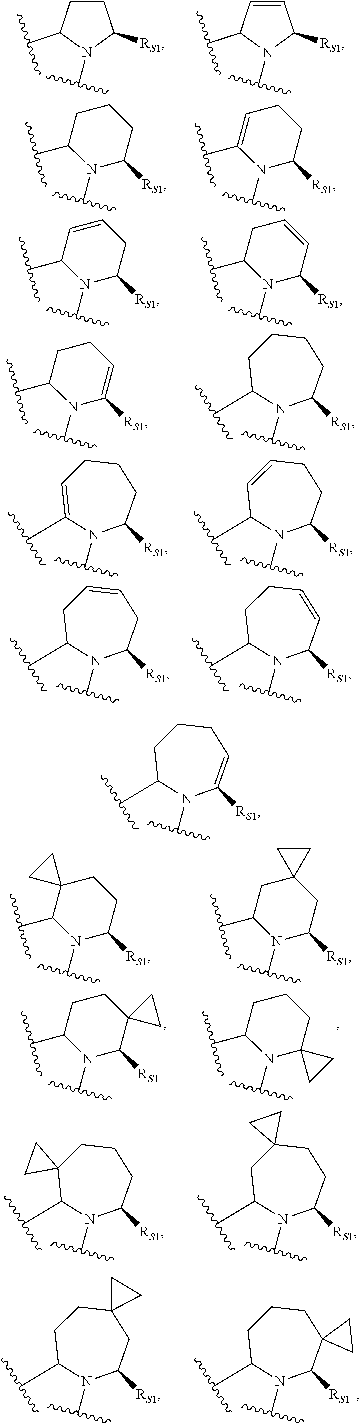

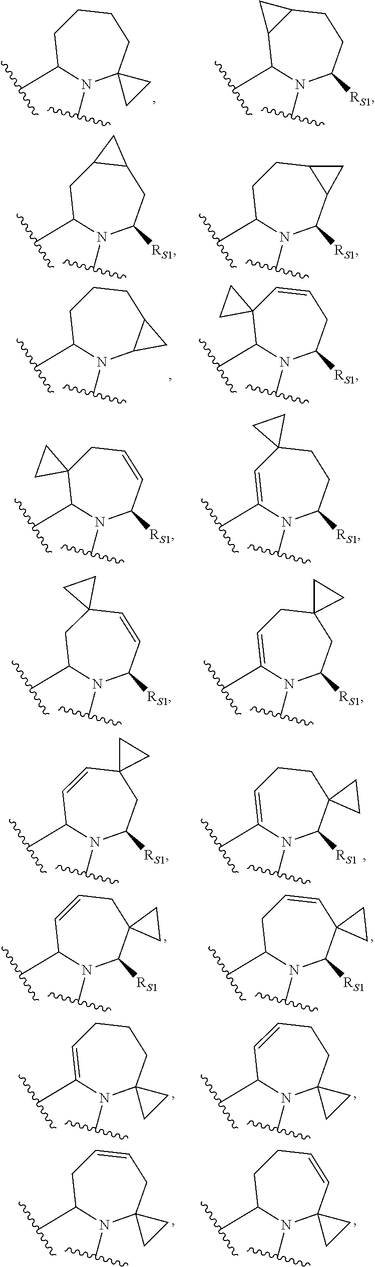

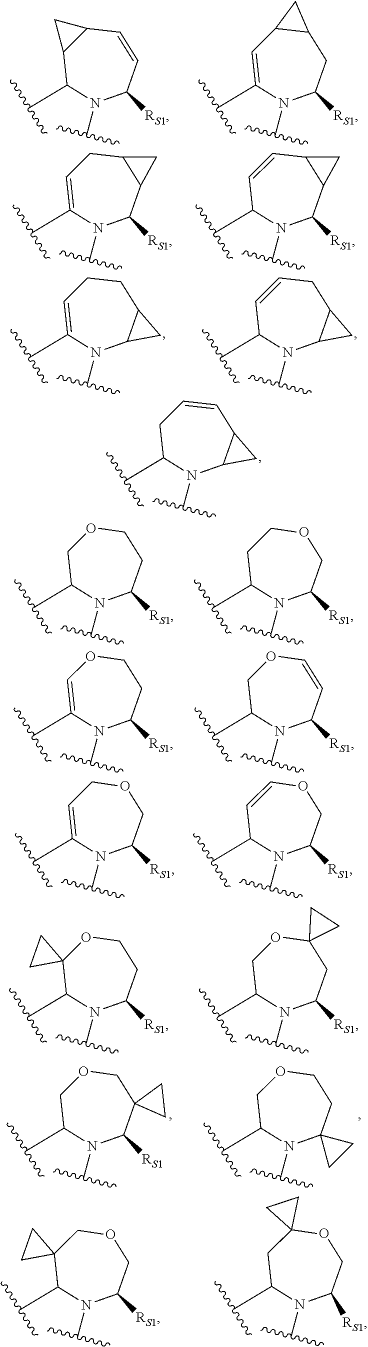

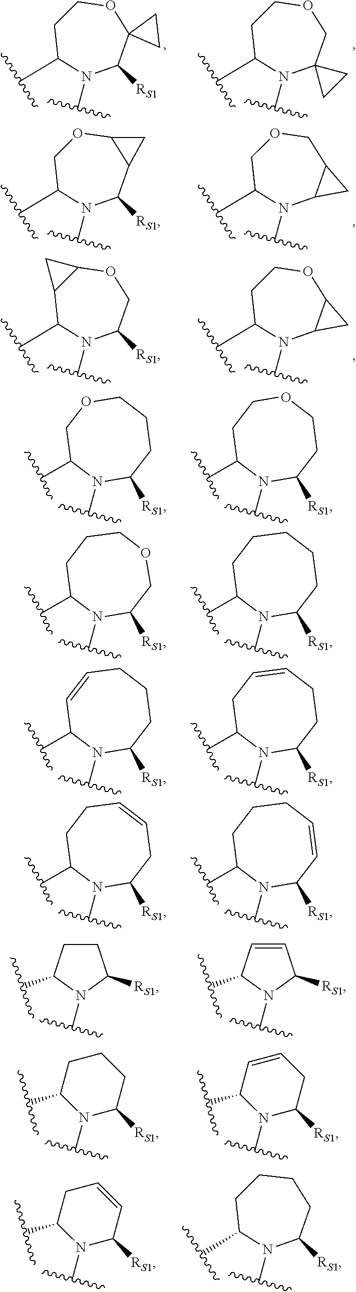

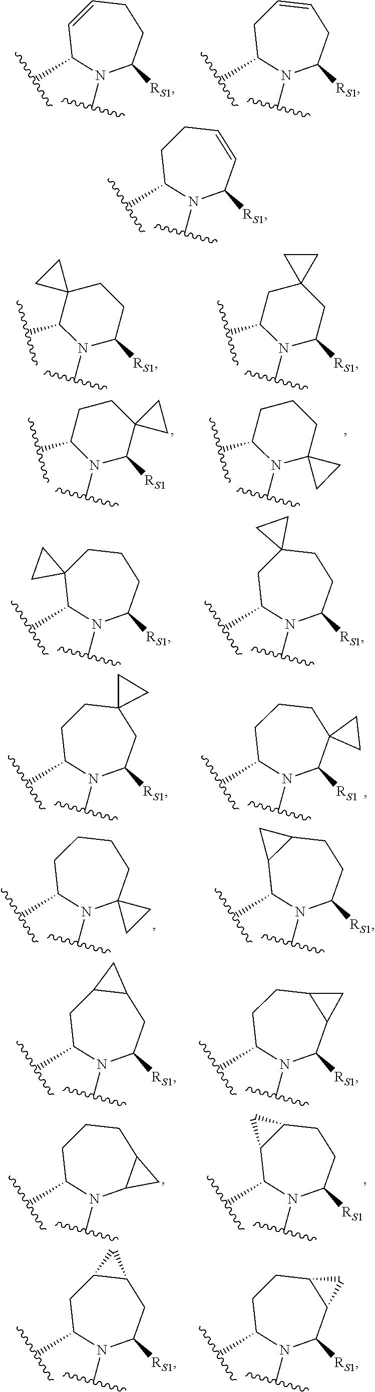

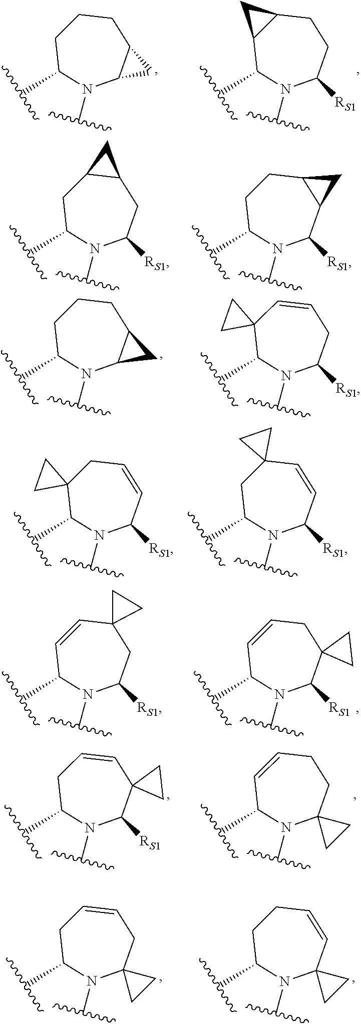

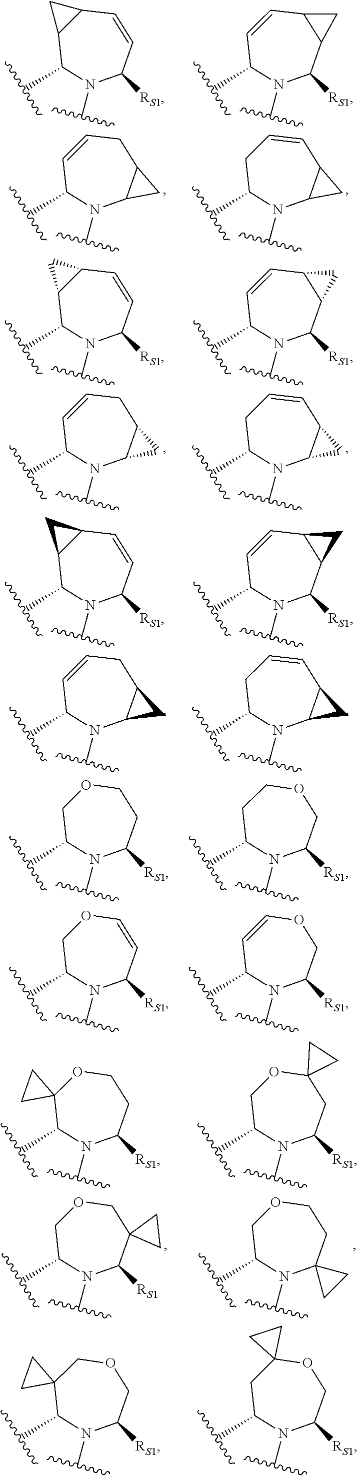

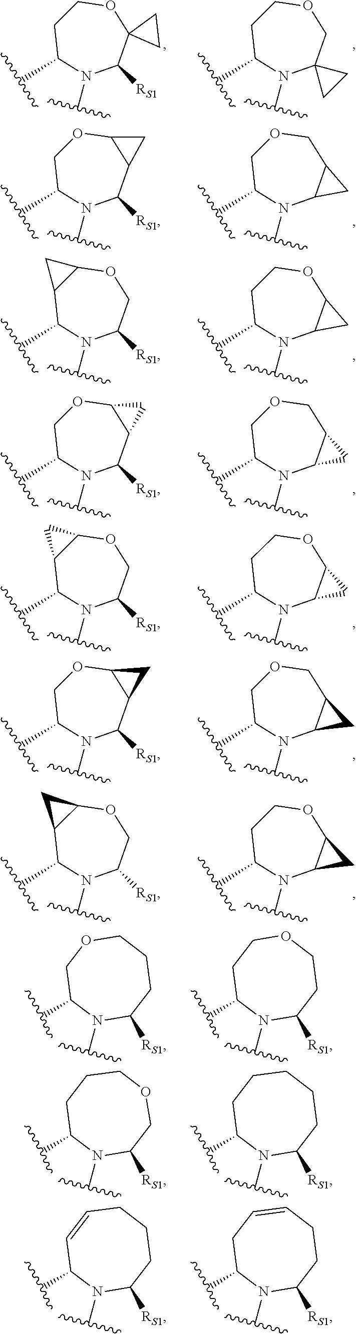

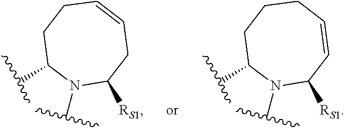

In some embodiments,

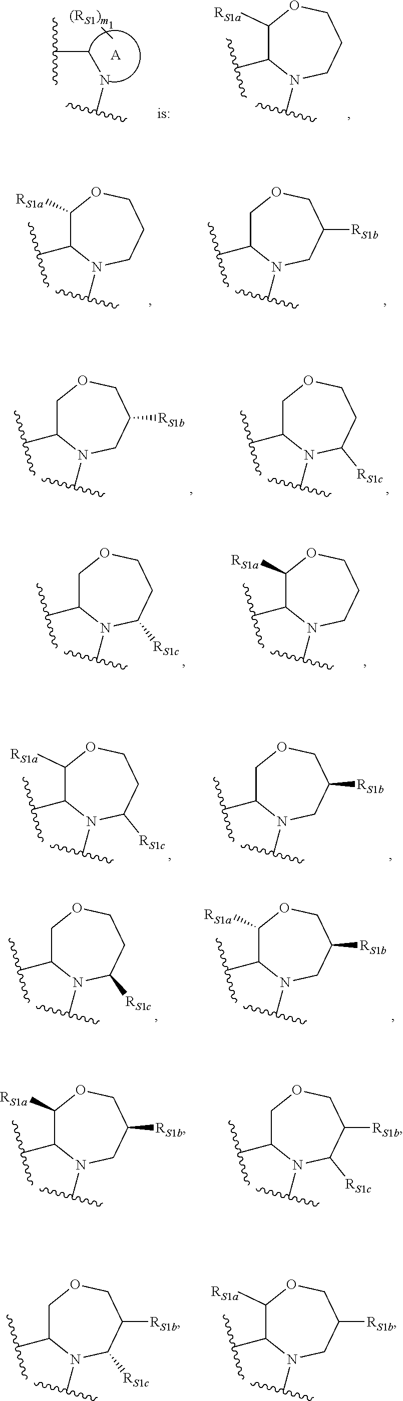

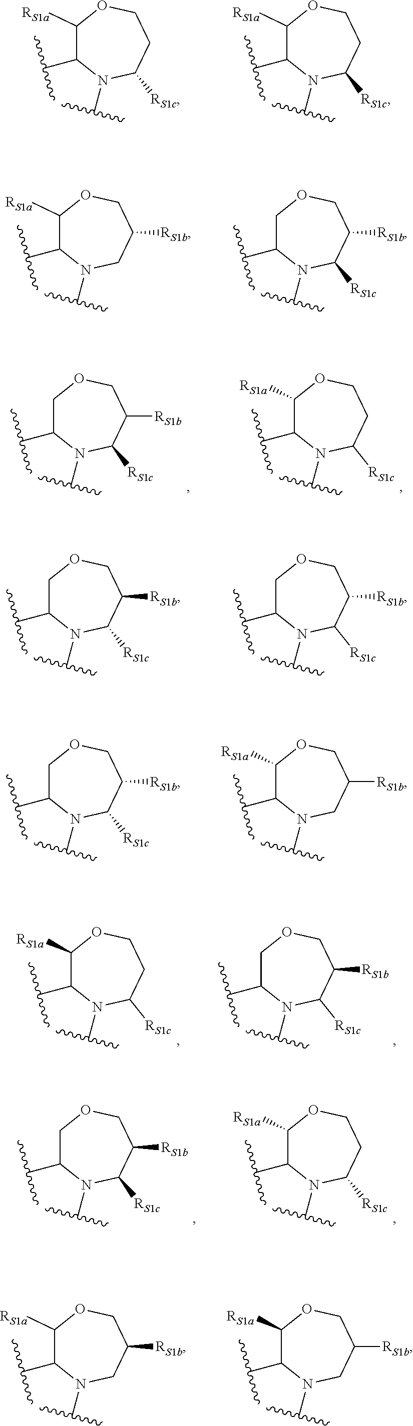

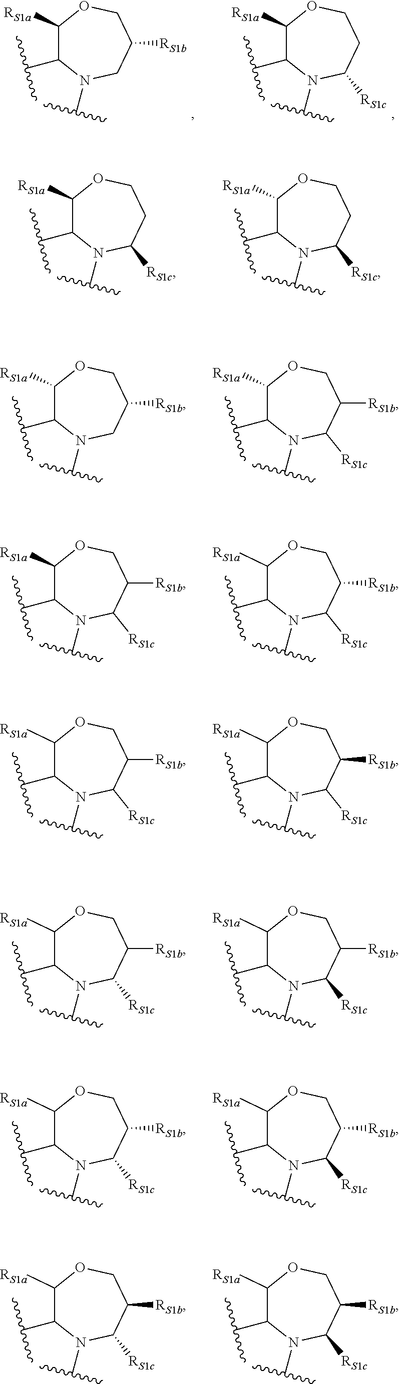

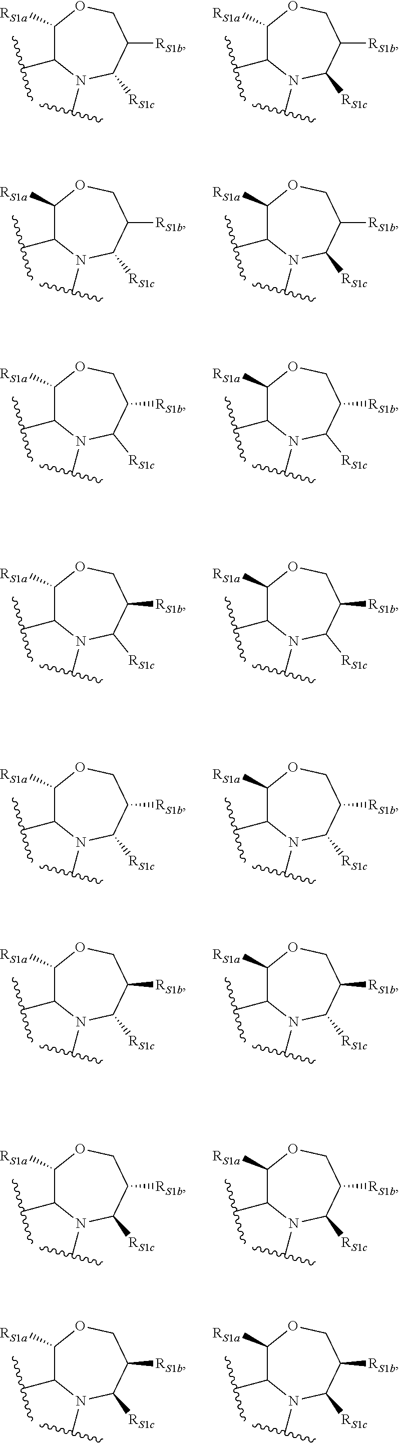

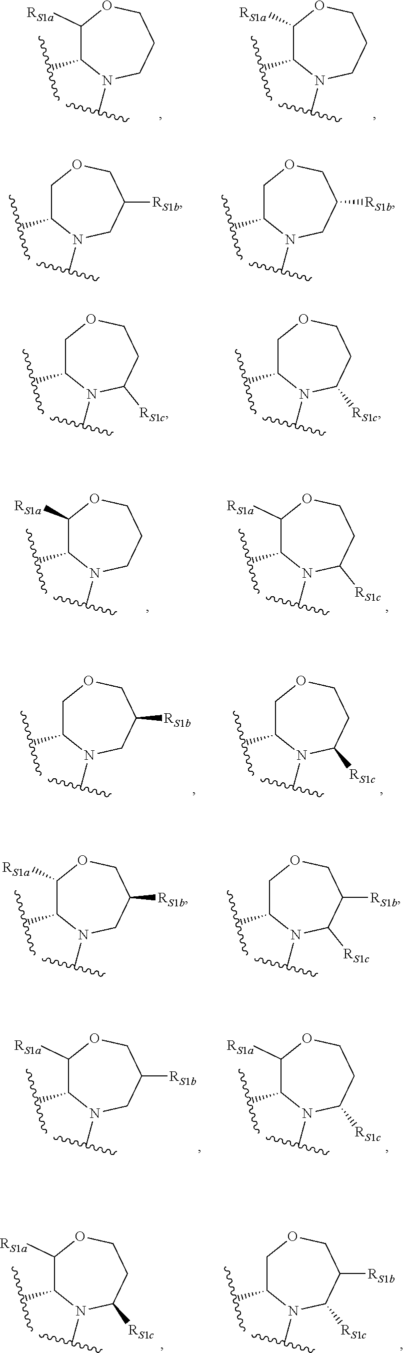

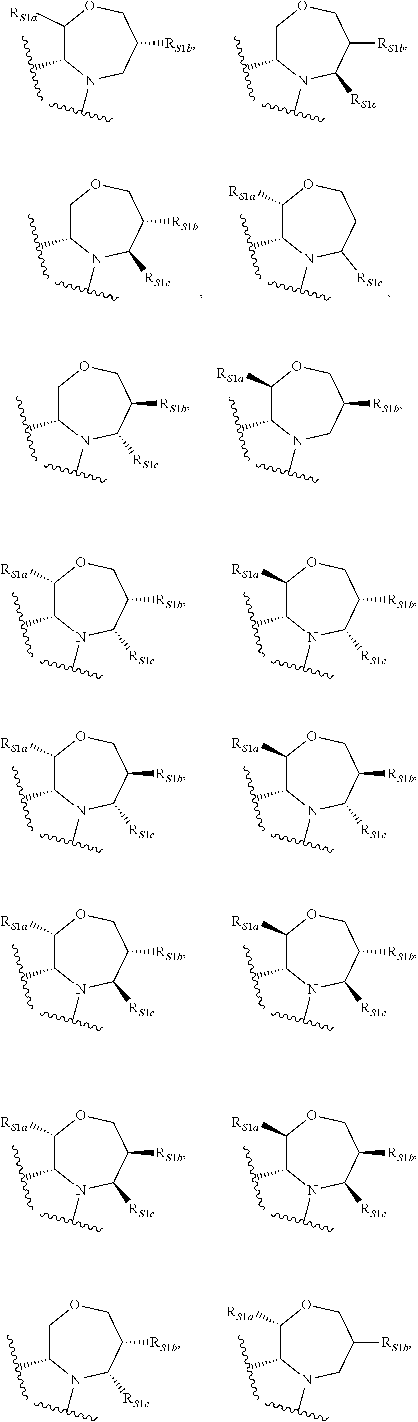

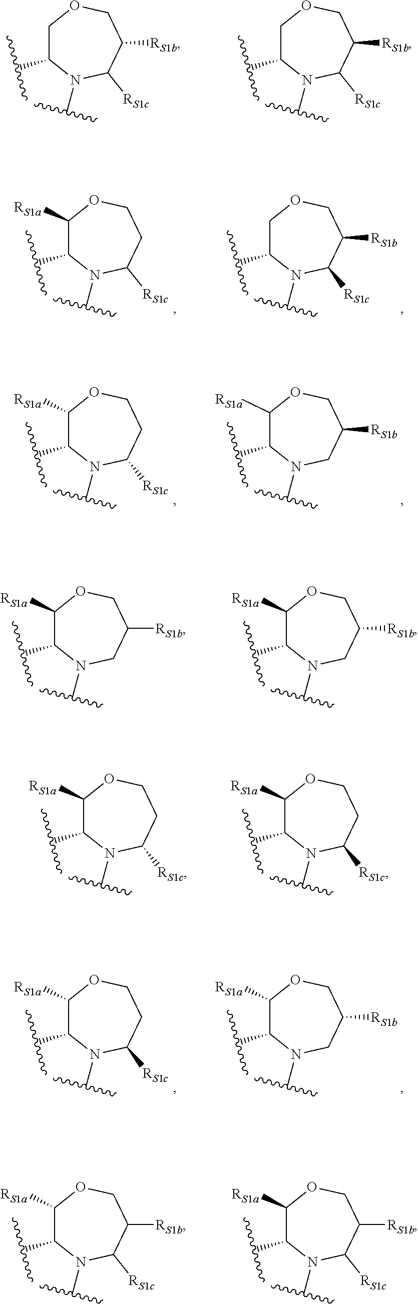

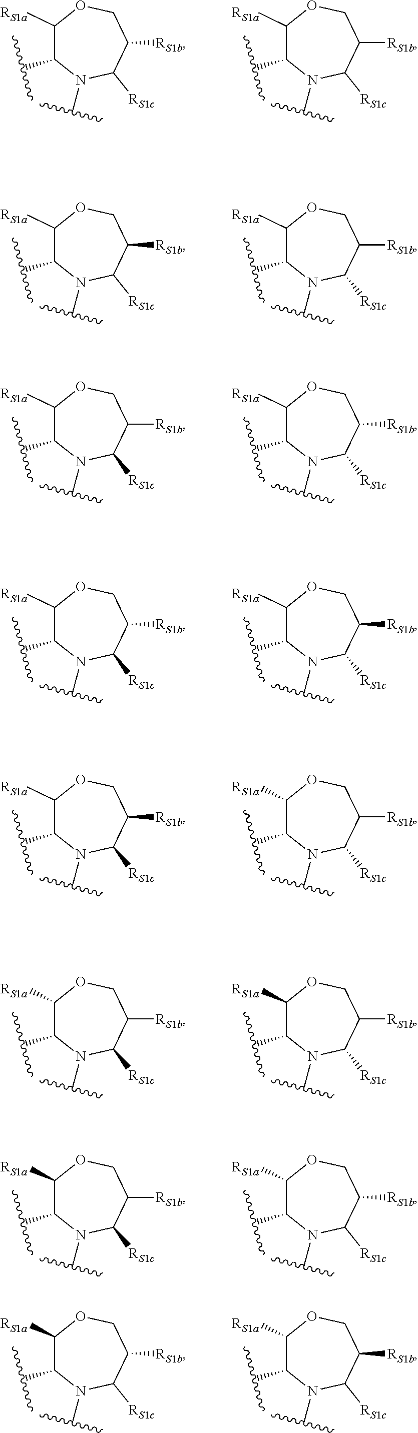

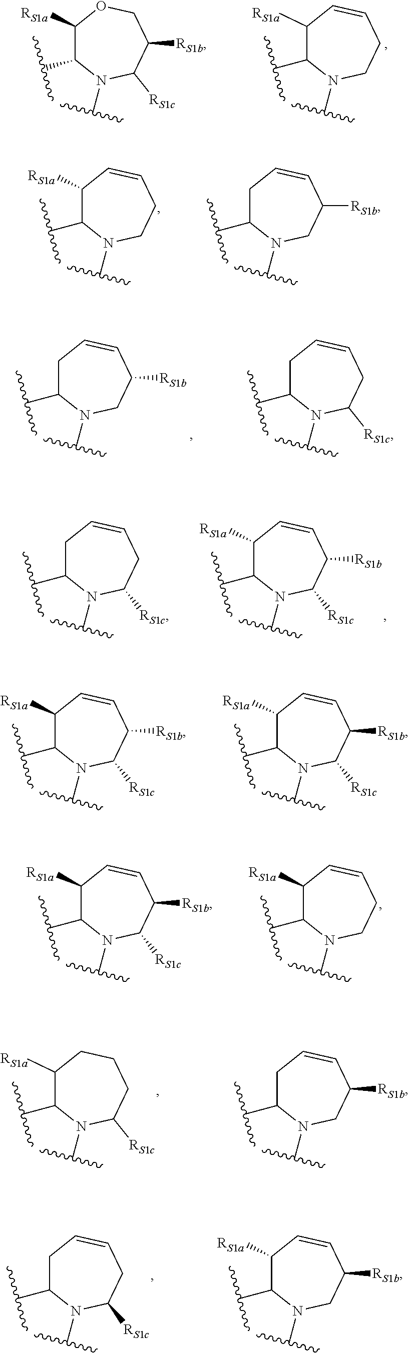

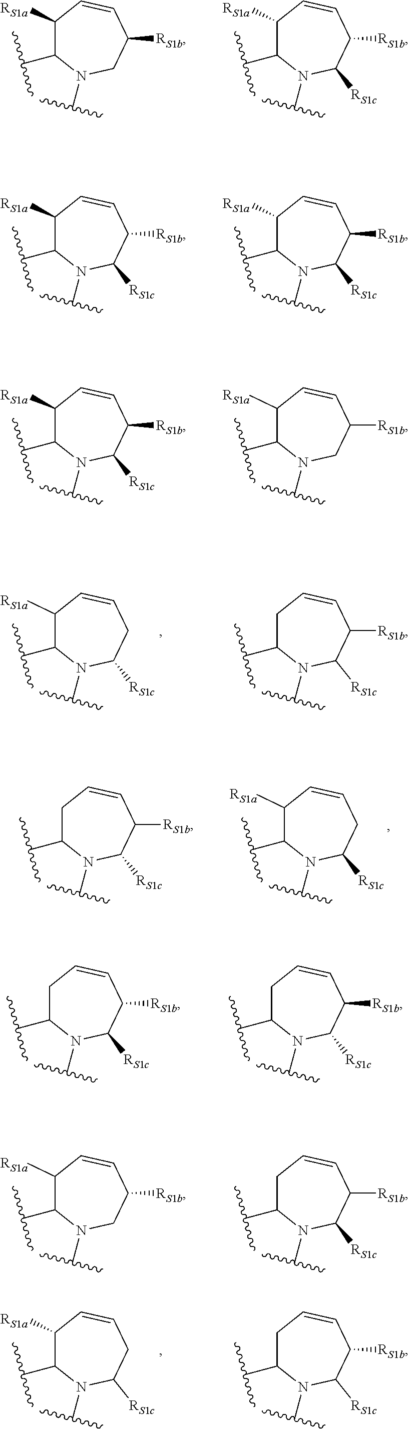

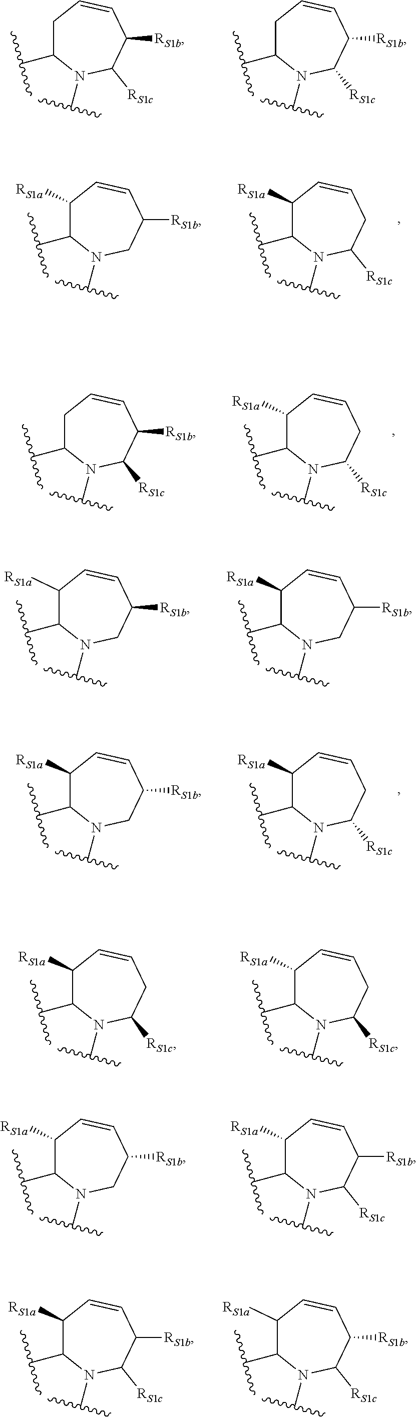

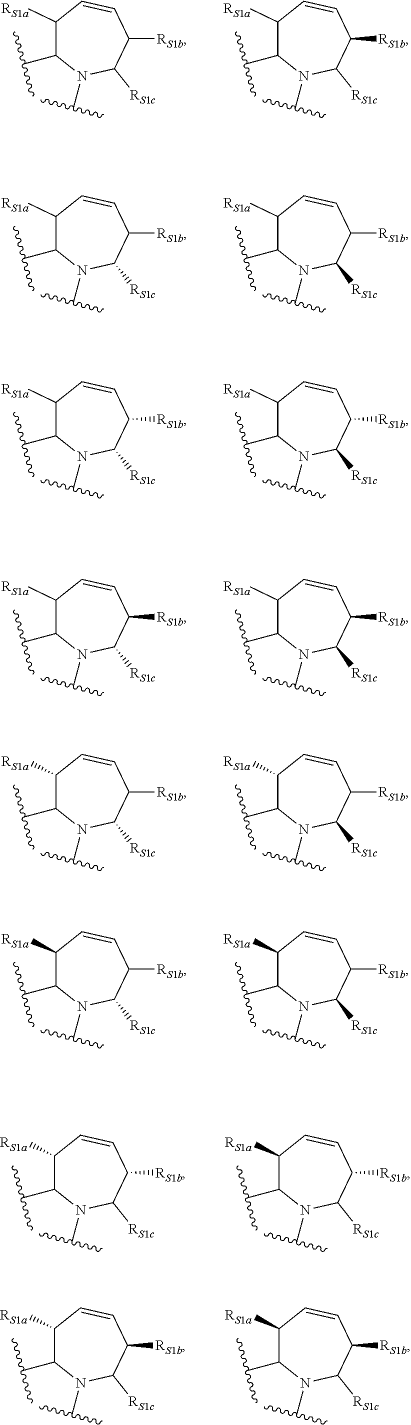

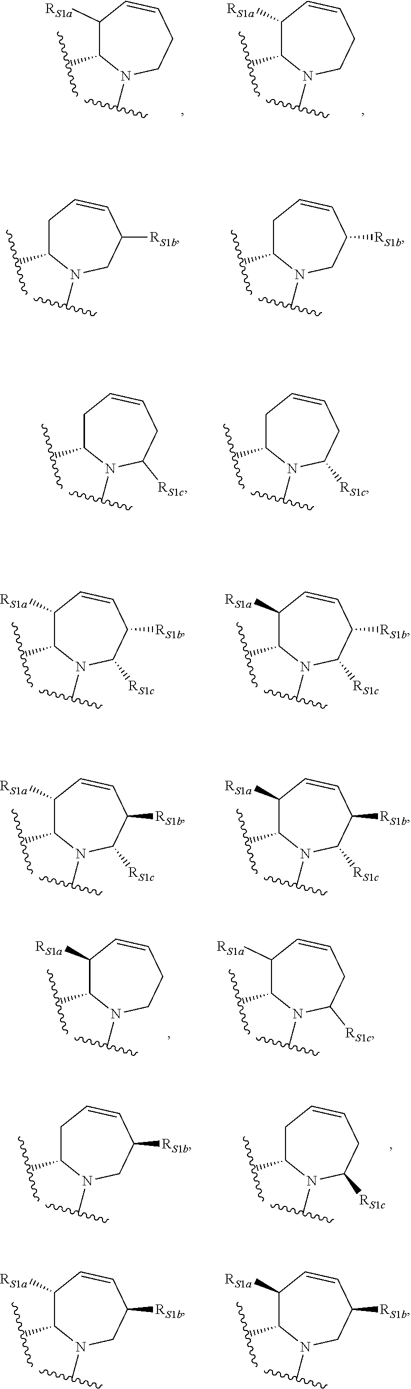

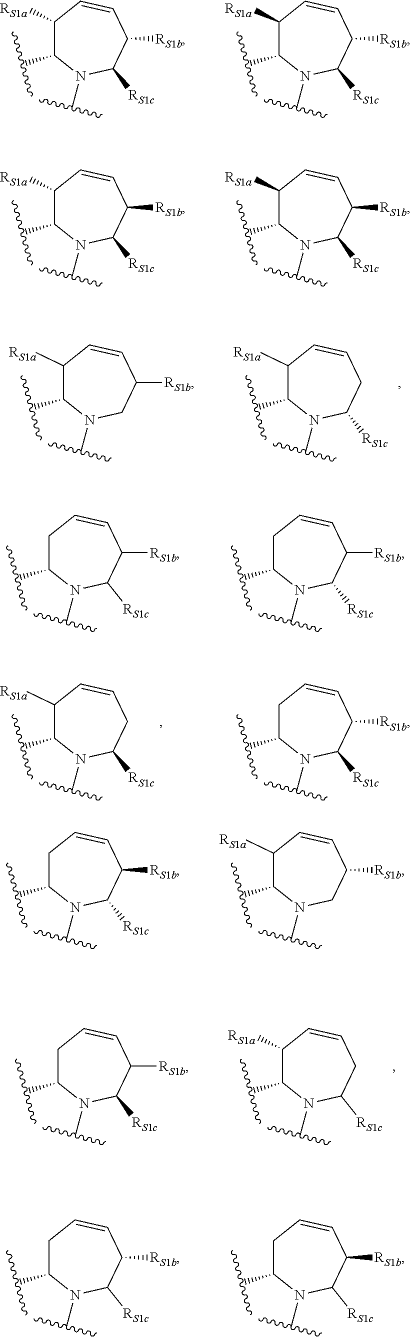

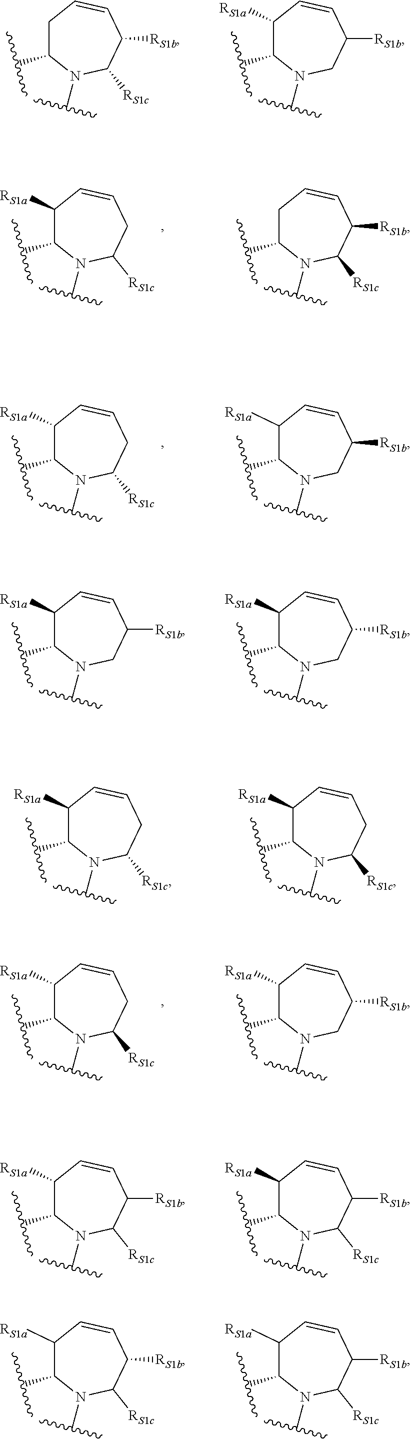

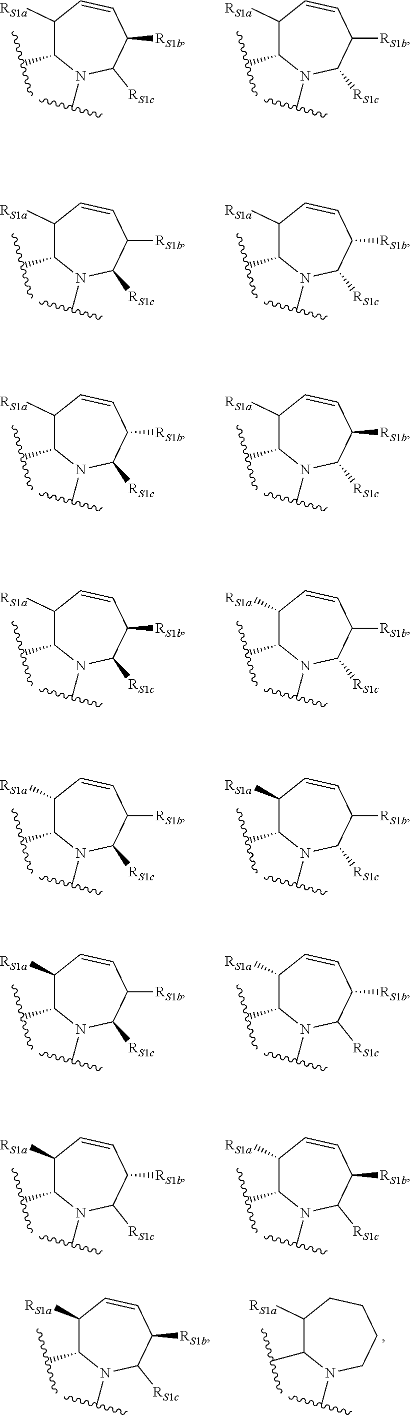

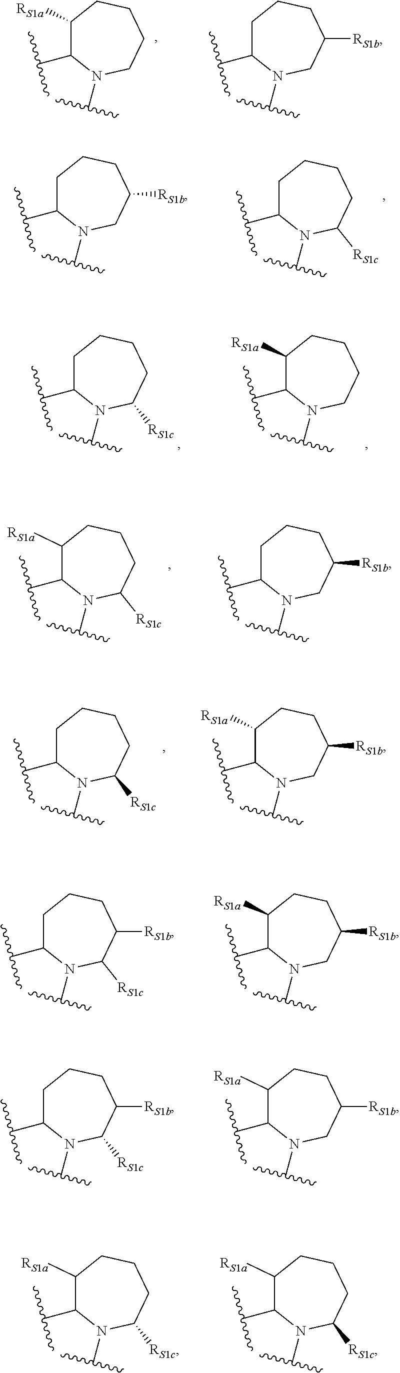

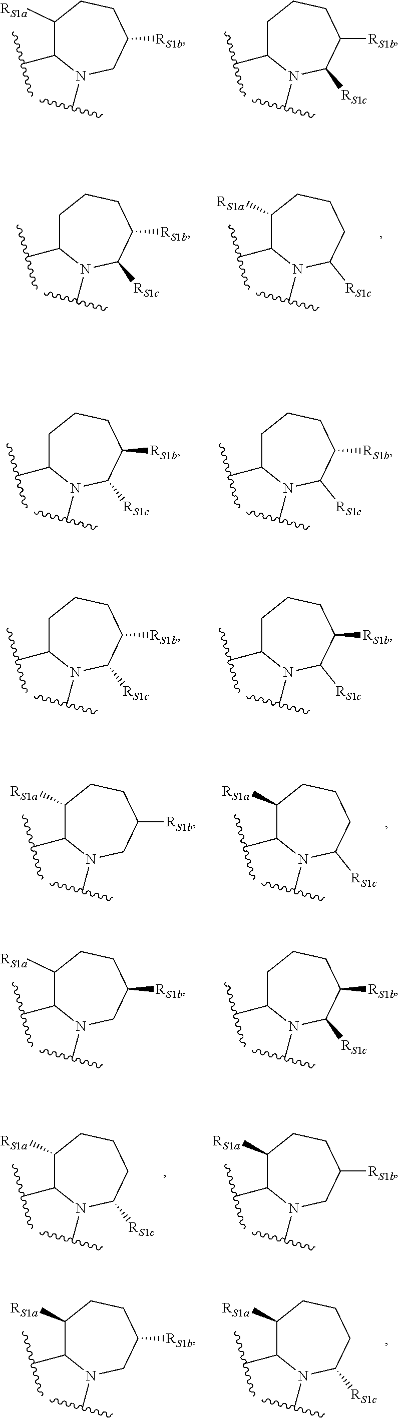

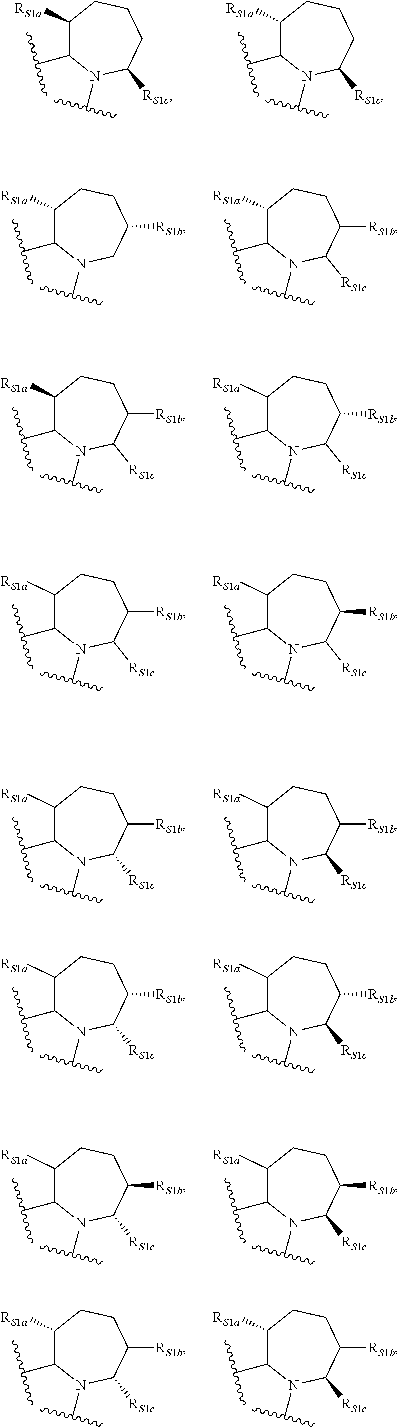

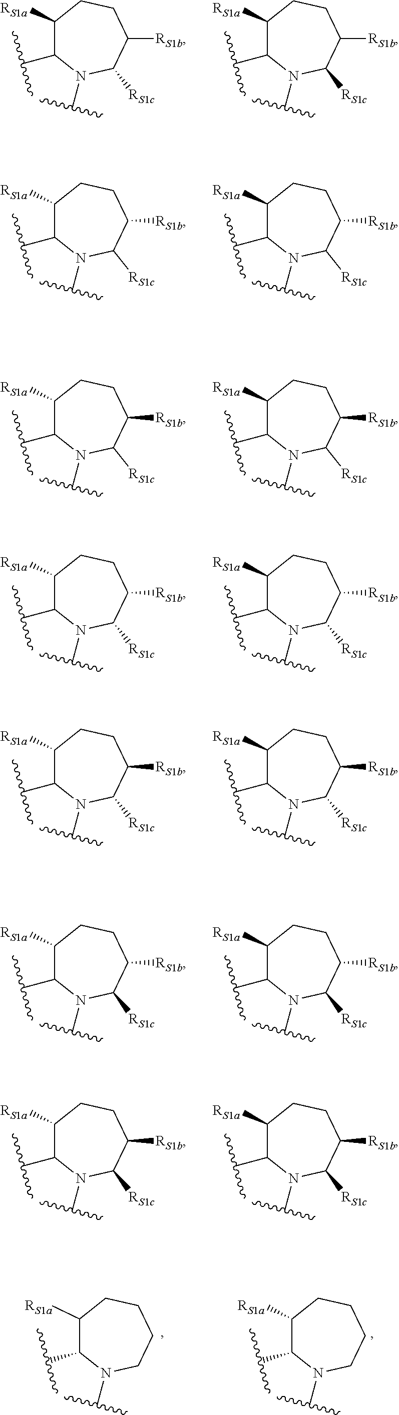

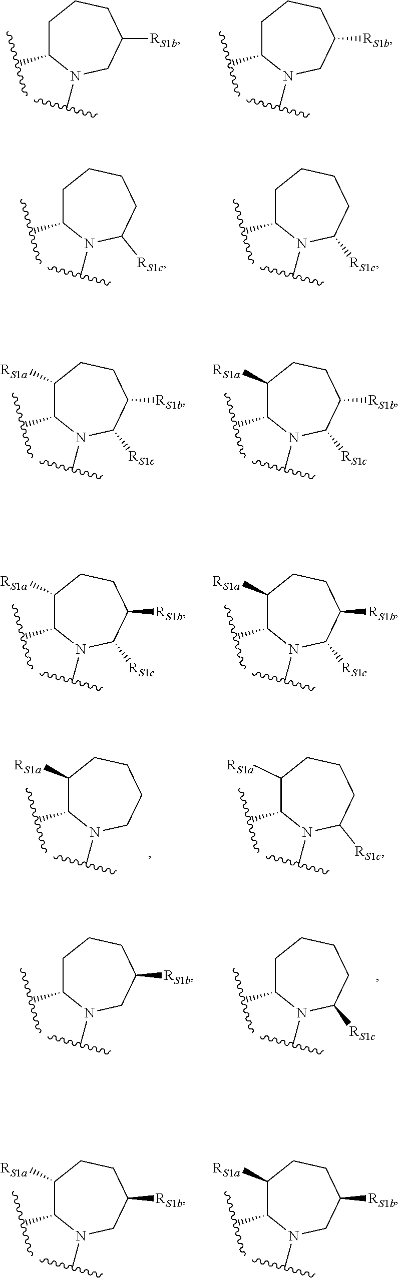

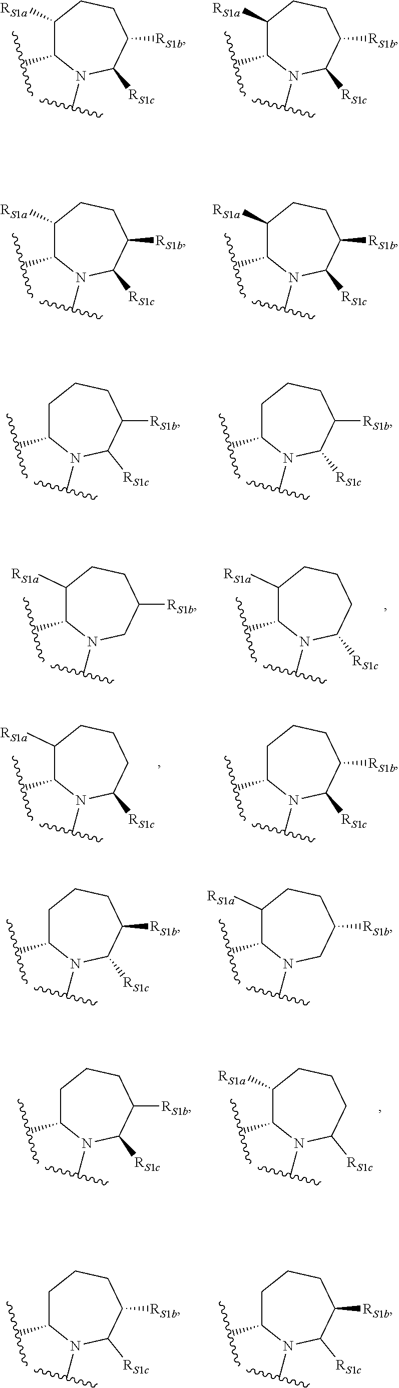

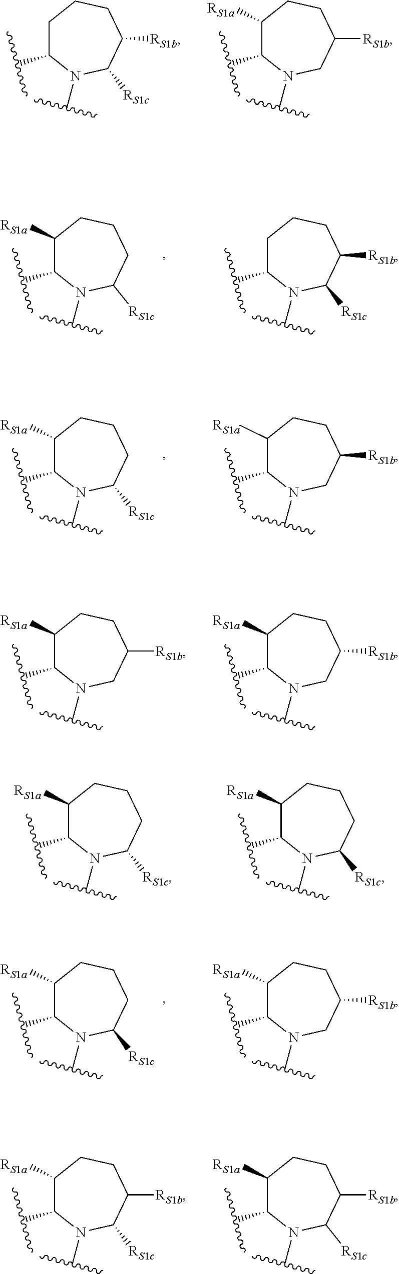

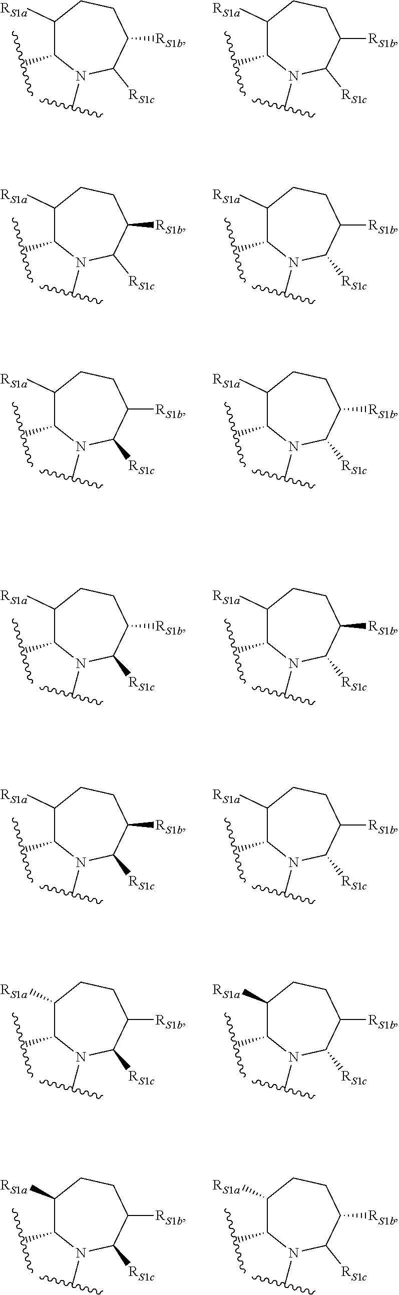

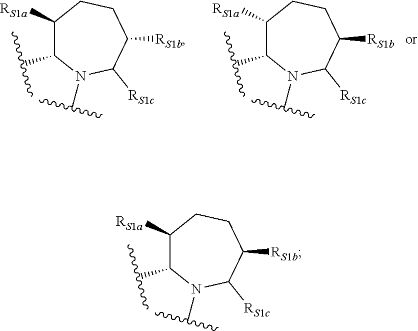

Wherein, the definition of R_{S1a}, R_{S1b}, or R_{S1c} is same as R_{S1}.

In some embodiments, R_{S1a} is deuterium, halogen, —C₁₋₆alkyl, —C₂₋₆alkenyl, —C₂₋₆alkynyl, haloC₁₋₆alkyl, haloC₁₋₆alkoxy, —CN, —NH₂, —NH(C₁₋₆alkyl), —N(C₁₋₆alkyl)₂, —OH, —OC₁₋₆alkyl or 3-6 membered cycloalkyl; wherein said-C₁₋₆alkyl, —C₂₋₆alkenyl, —C₂₋₆alkynyl or 3-6 membered cycloalkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl. In some embodiments, R_{S1a} is —F, —OH, —OCH₃, —CN, —CH₂F, —CF₃, —CH₂OCH₃, —CH₂CN, —CHF₂, —CD₃, —NH₂ or —CH₃.

In some embodiments, R_{S1b} is deuterium, halogen, —C₁₋₆alkyl, —C₂₋₆alkenyl, —C₂₋₆alkynyl, haloC₁₋₆alkyl, haloC₁₋₆alkoxy, —CN, —NH₂, —NH(C₁₋₆alkyl), —N(C₁₋₆alkyl)₂, —OH, —OC₁₋₆alkyl or 3-6 membered cycloalkyl; wherein said-C₁₋₆alkyl, —C₂₋₆alkenyl, —C₂₋₆alkynyl or 3-6 membered cycloalkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl. In some embodiments, R_{S1b} is —F, —OH, —OCH₃, —CN, —CH₂F, —CF₃, —CH₂OCH₃, —CH₂CN, —CHF₂, —CD₃, —NH₂ or —CH₃.

In some embodiments, R_{S1c} is deuterium, halogen, —C₁₋₆alkyl, —C₂₋₆alkenyl, —C₂₋₆alkynyl, haloC₁₋₆alkyl, haloC₁₋₆alkoxy, —CN, —NH₂, —NH(C₁₋₆alkyl), —N(C₁₋₆alkyl)₂, —OH, —OC₁₋₆alkyl or 3-6 membered cycloalkyl; wherein said-C₁₋₆alkyl, —C₂₋₆alkenyl, —C₂₋₆alkynyl or 3-6 membered cycloalkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl. In some embodiments, R_{S1c} is —F, —OH, —OCH₃, —CN, —CH₂F, —CF₃, —CH₂OCH₃, —CH₂CN, —CHF₂, —CD₃, —NH₂ or —CH₃.

In some embodiments,

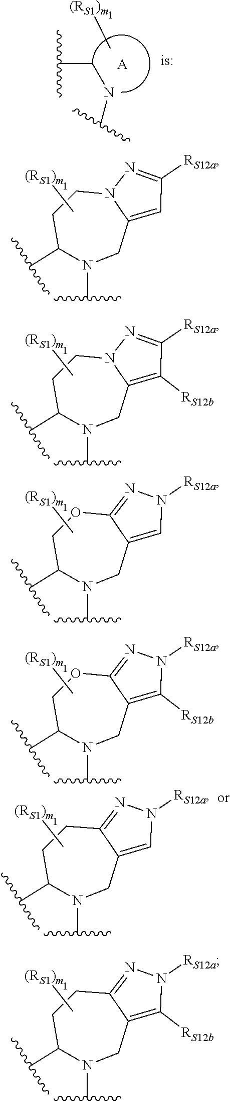

R_{S12a} is deuterium, halogen, —C₁₋₆alkyl, —C₂₋₆alkenyl, —C₂₋₆alkynyl, haloC₁₋₆alkyl, haloC₁₋₆alkoxy, —CN, —NH₂, —NH(C₁₋₆alkyl), —N(C₁₋₆alkyl)₂, —OH, —OC₁₋₆alkyl or 3-6 membered cycloalkyl; wherein said —C₁₋₆alkyl, —C₂₋₆alkenyl, —C₂₋₆alkynyl or 3-6 membered cycloalkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl. In some embodiments, R_{S12b} is —F, —OH, —OCH₃, —CN, —CH₂F, —CF₃, —CH₂OCH₃, —CH₂CN, —CHF₂, —CD₃, —NH₂ or —CH₃.
R_{S12b} is deuterium, halogen, —C₁₋₆alkyl, —C₂₋₆alkenyl, —C₂₋₆alkynyl, haloC₁₋₆alkyl, haloC₁₋₆alkoxy, —CN, —NH₂, —NH(C₁₋₆alkyl), —N(C₁₋₆alkyl)₂, —OH, —OC₁₋₆alkyl or 3-6 membered cycloalkyl; wherein said —C₁₋₆alkyl, —C₂₋₆alkenyl, —C₂₋₆alkynyl or 3-6 membered cycloalkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl. In some embodiments, R_{S12b} is —F, —OH, —OCH₃, —CN, —CH₂F, —CF₃, —CH₂OCH₃, —CH₂CN, —CHF₂, —CD₃, —NH₂ or —CH₃.

In some embodiments, R_{S12a} is —NH(C₁₋₆alkyl), or —N(C₁₋₆alkyl)₂; R_{S12b} is halogen, —C₁₋₆alkyl, —CN, or 3-6 membered cycloalkyl;
In some embodiments, R_{S12a} is —NH(methyl), or —N(methyl)₂; R_{S12b} is —F, —Cl, methyl, —CN, or 3 membered cycloalkyl.

In some embodiments,

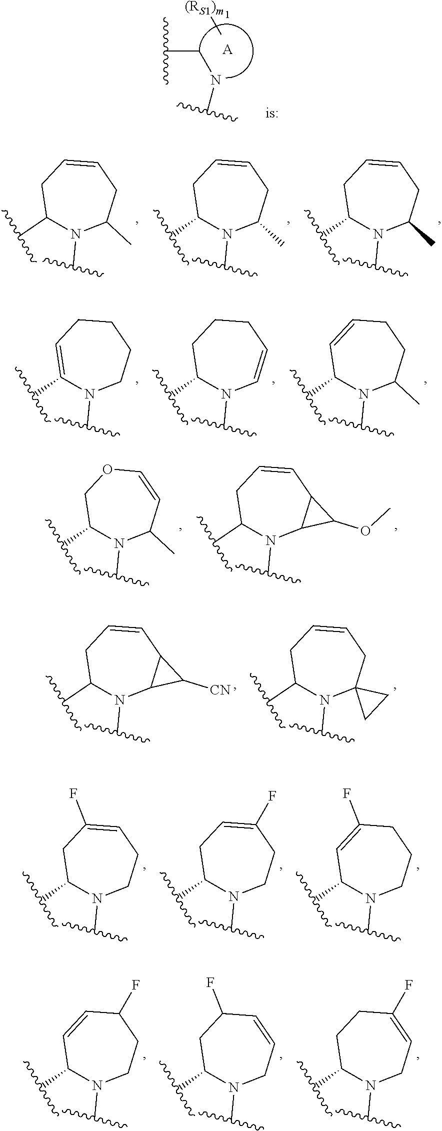

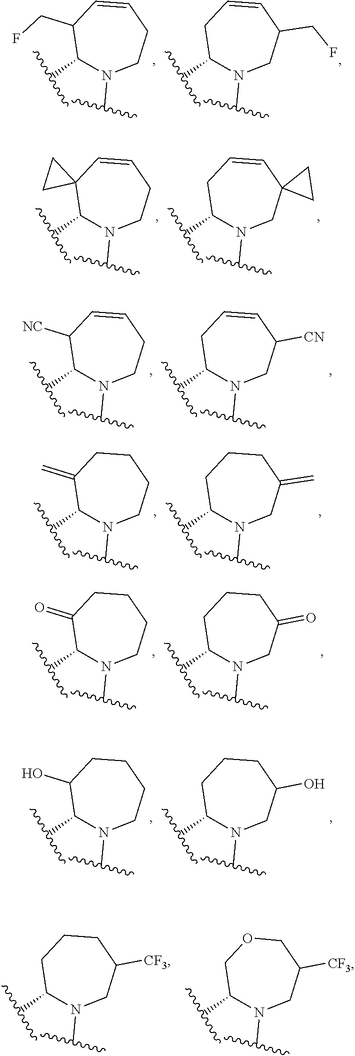

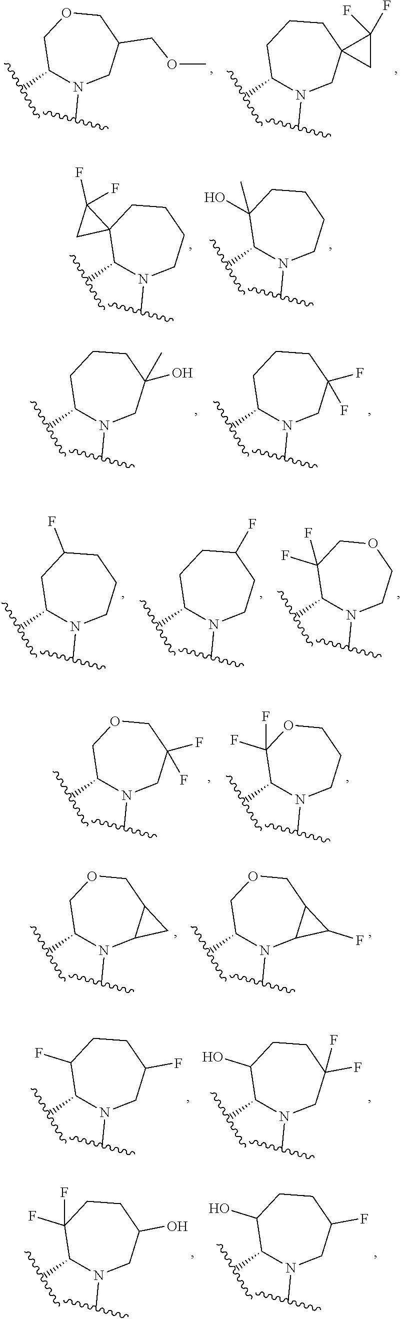

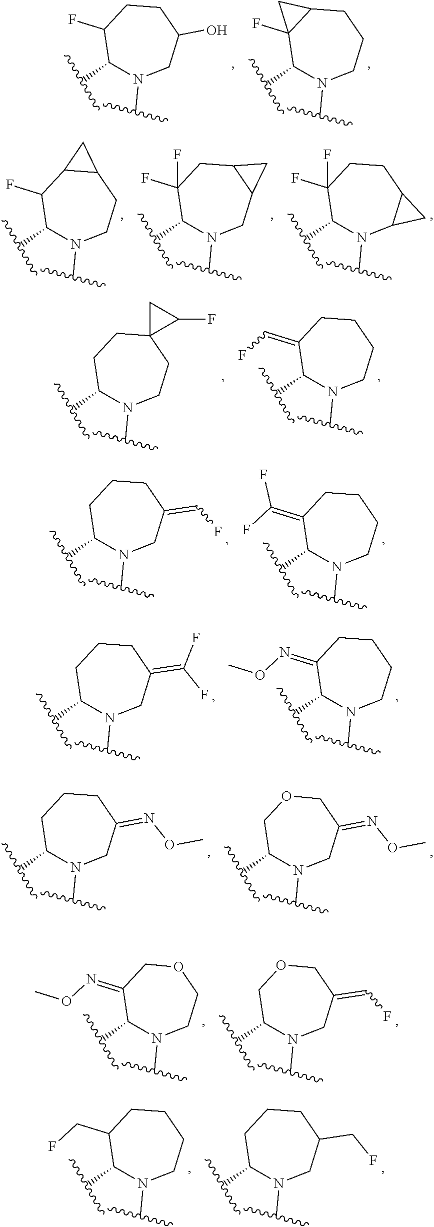

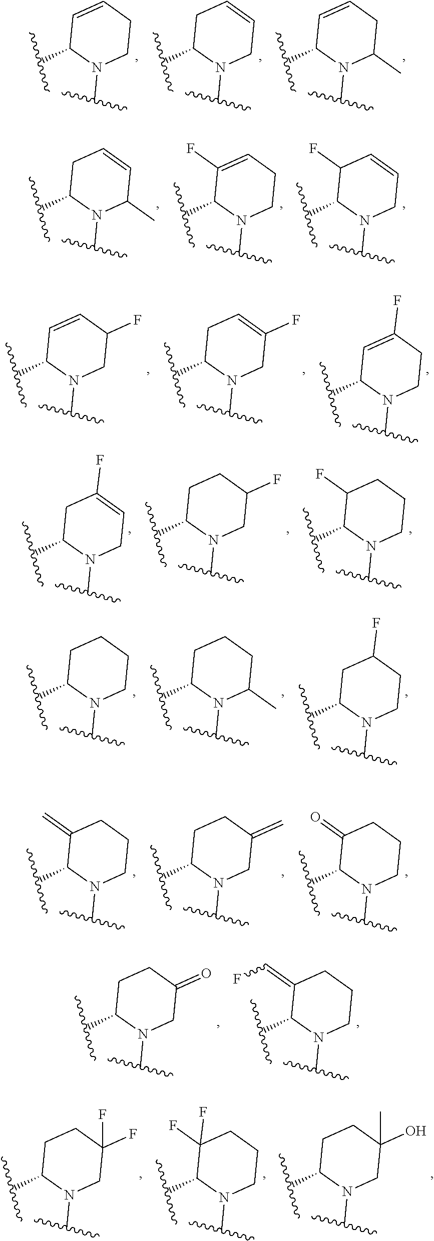

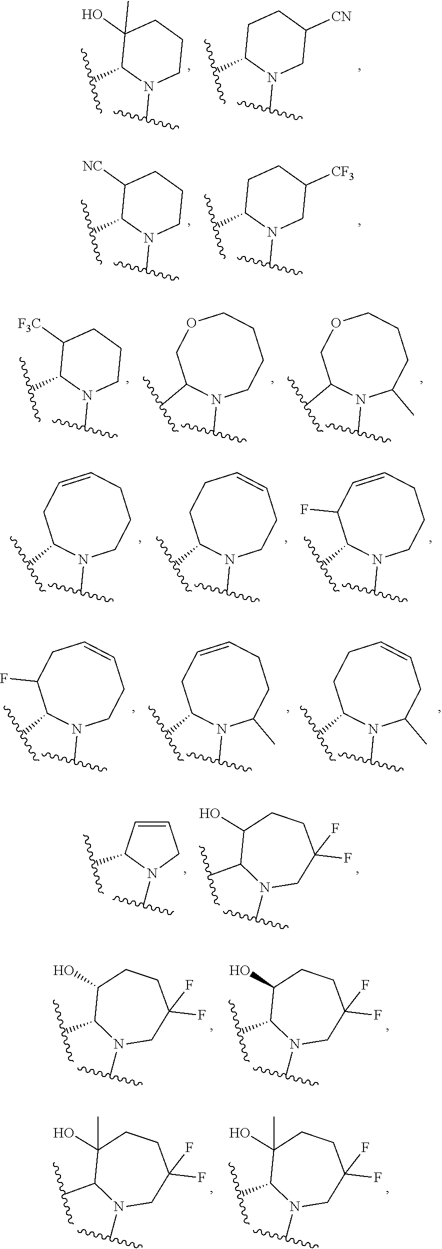

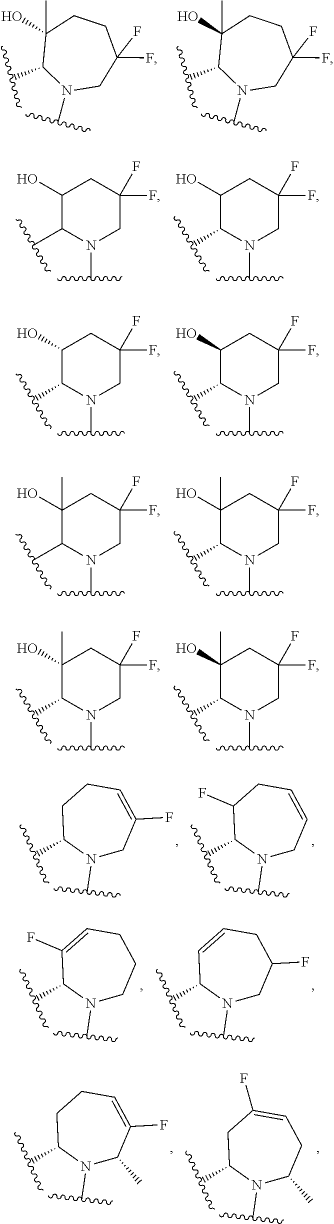

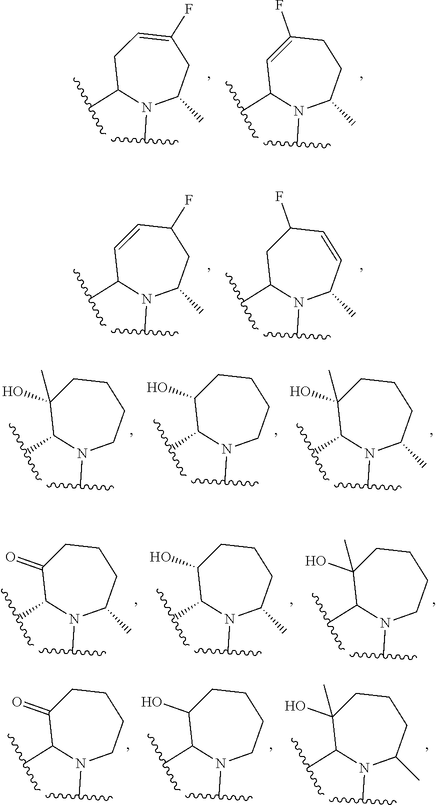

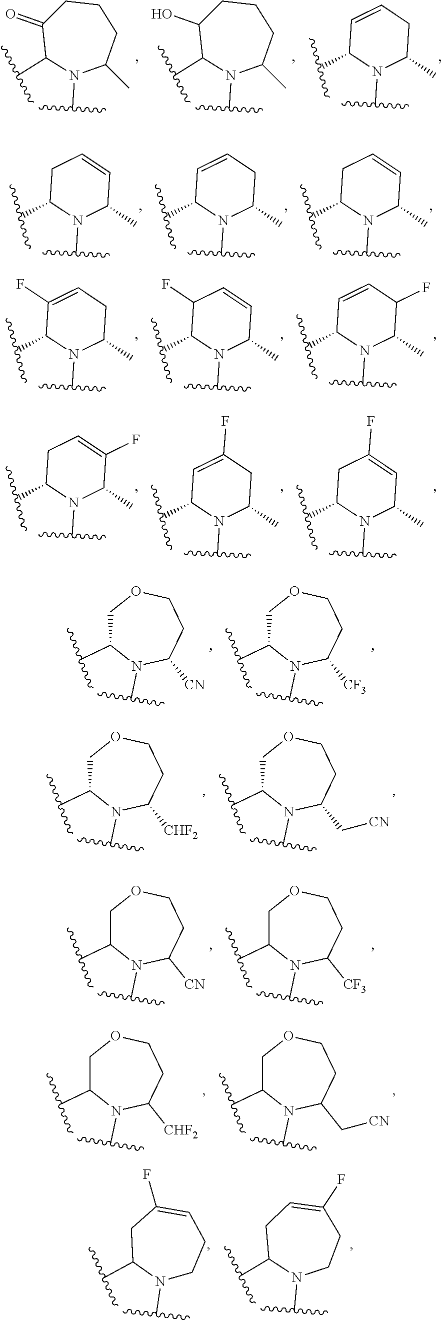

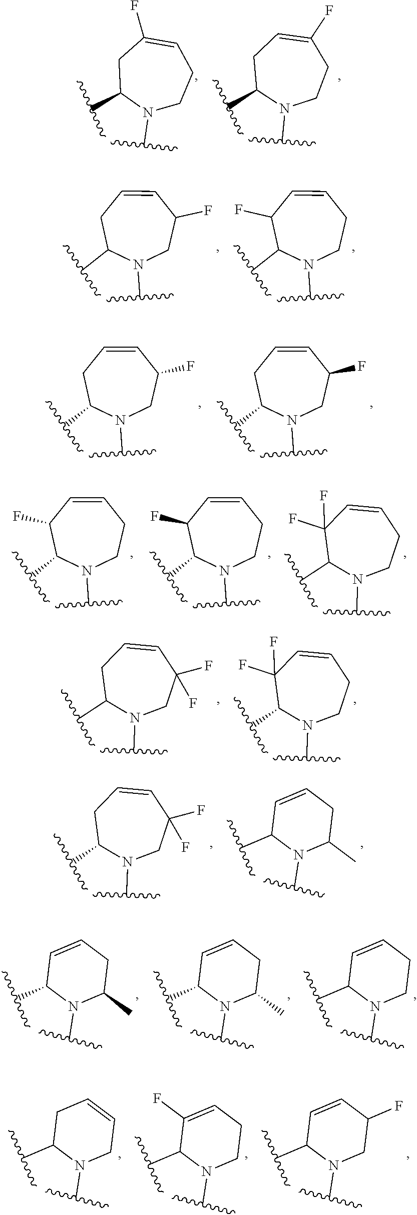

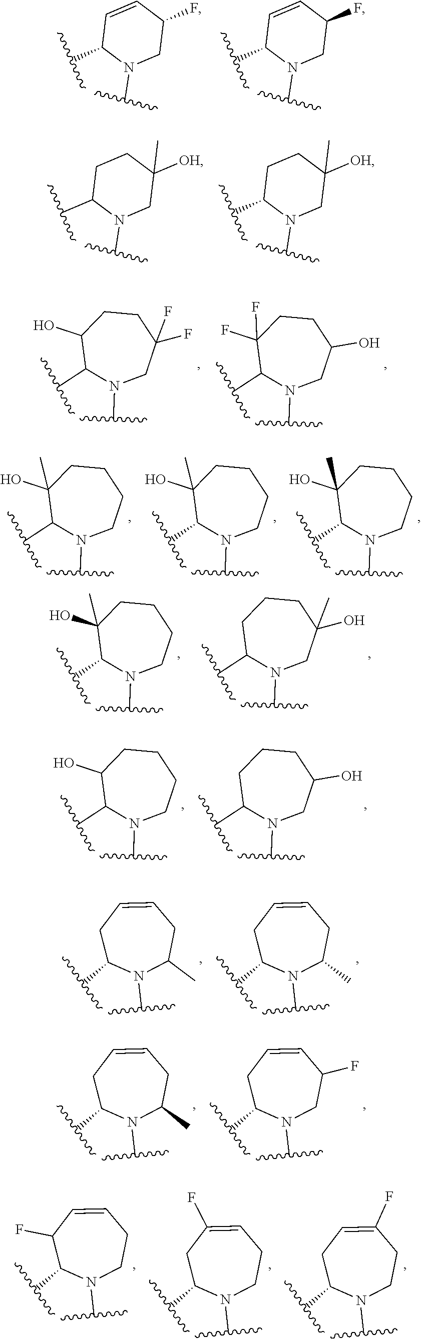

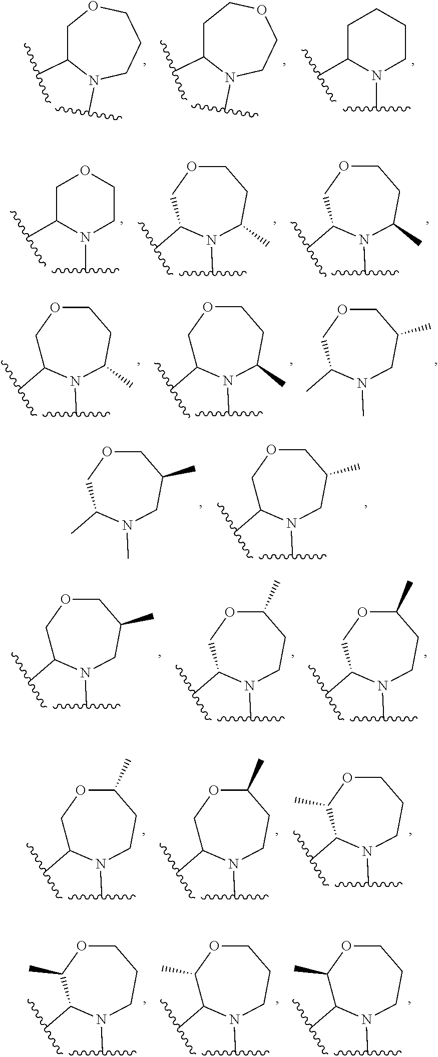

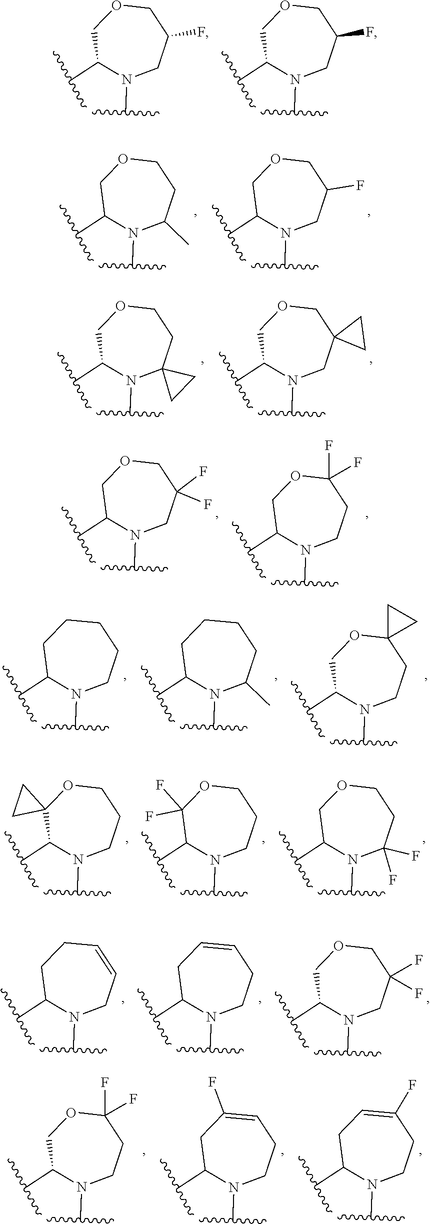

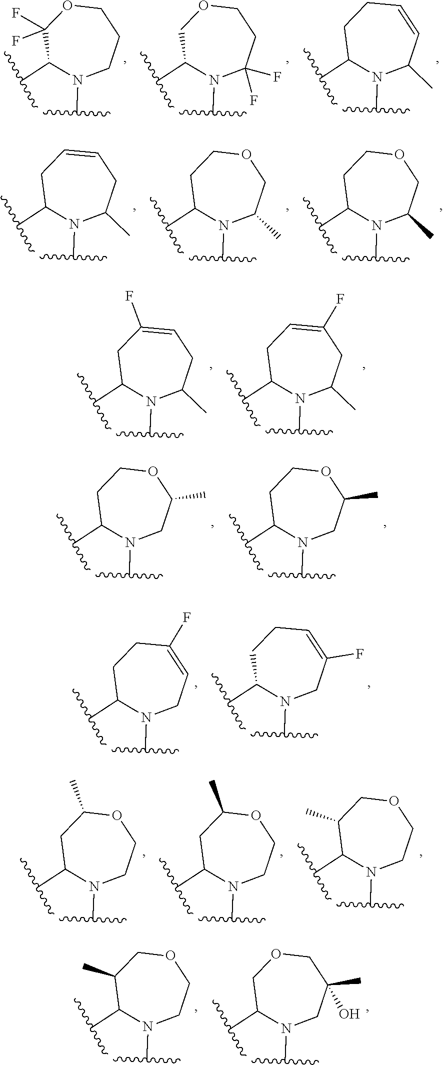

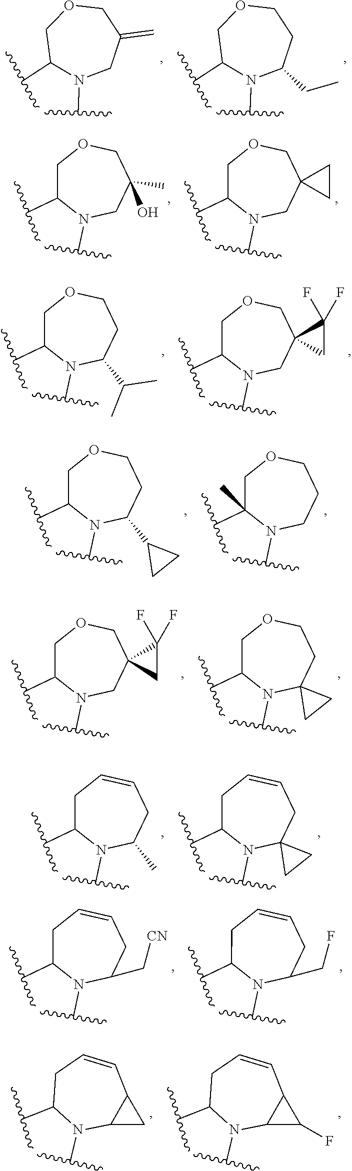

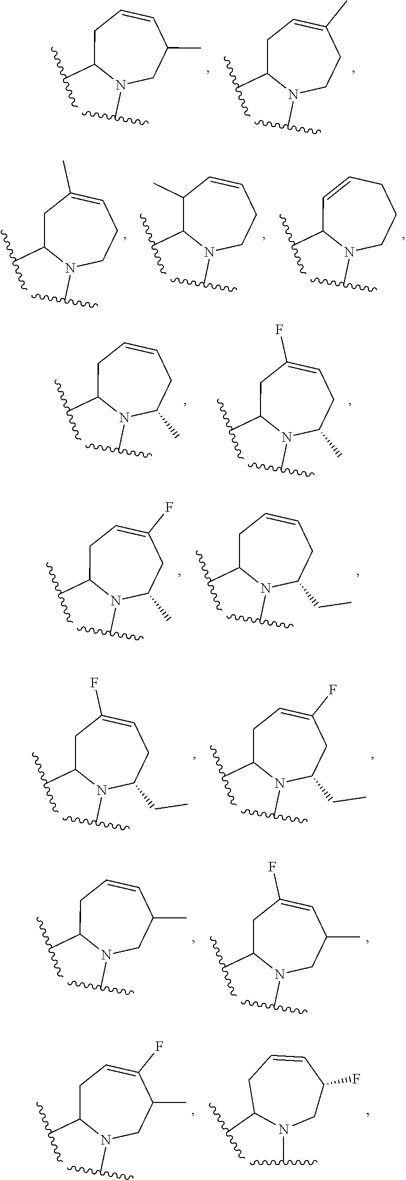

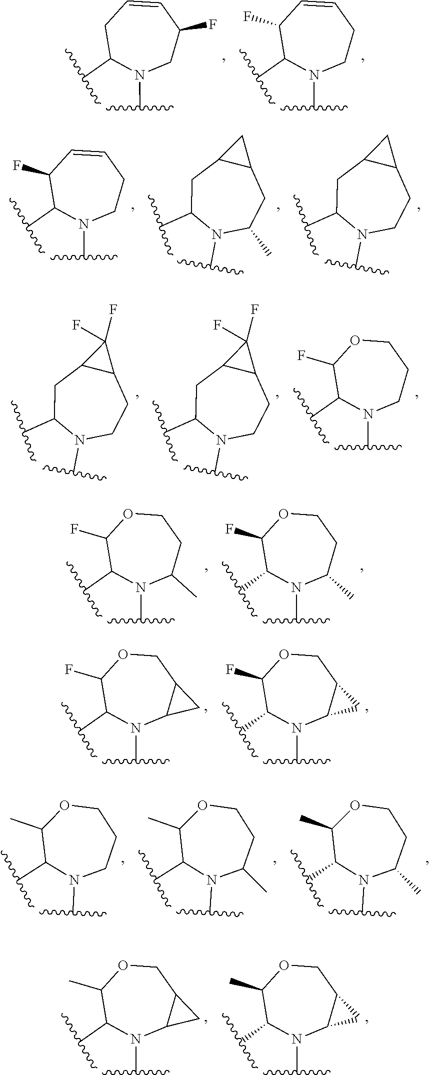

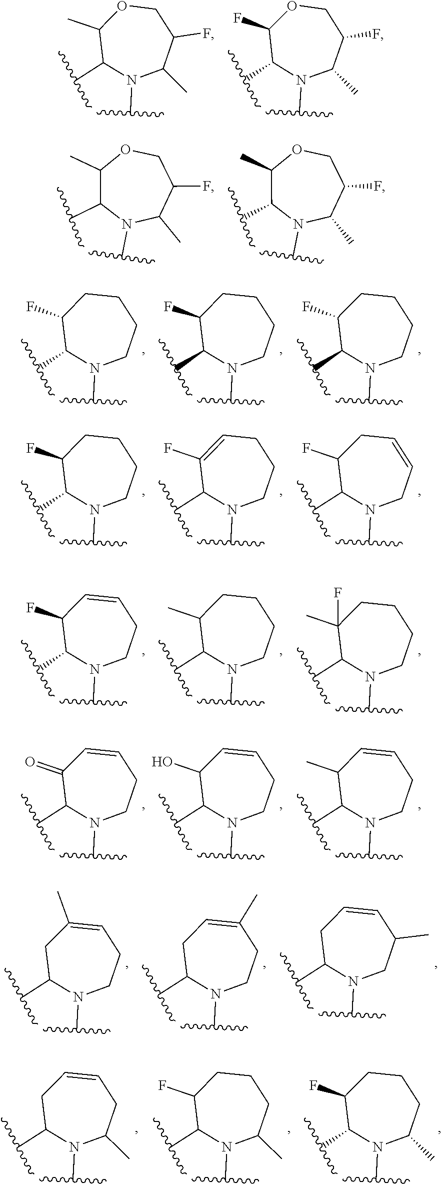

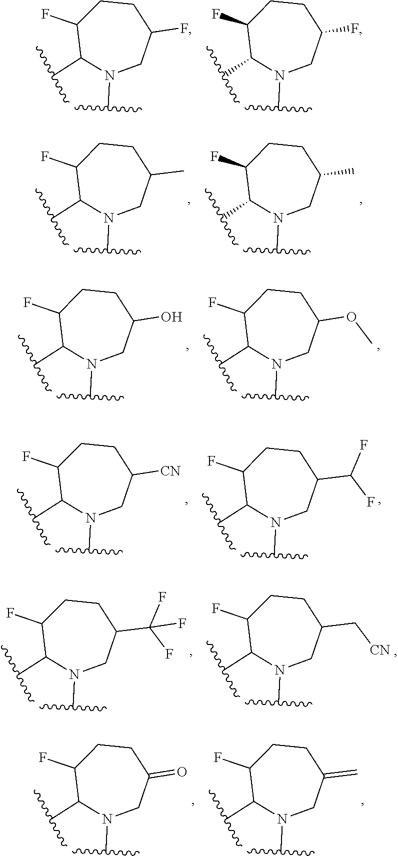

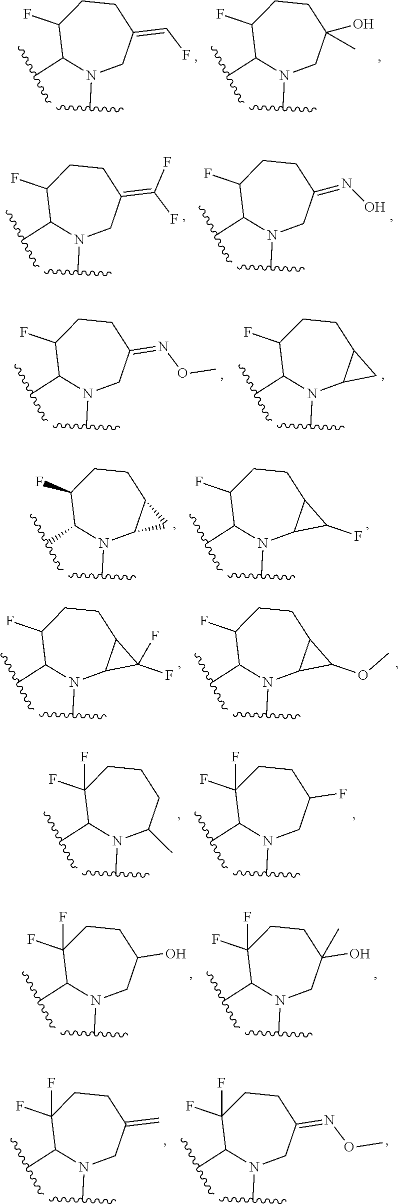

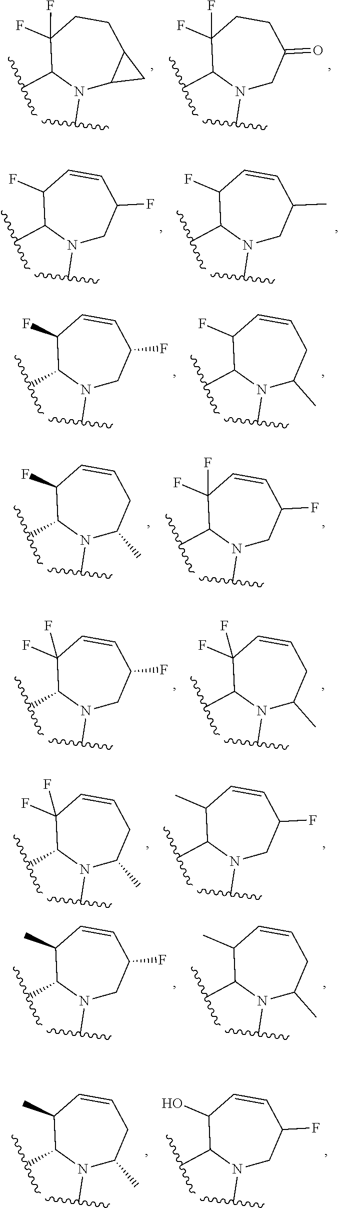

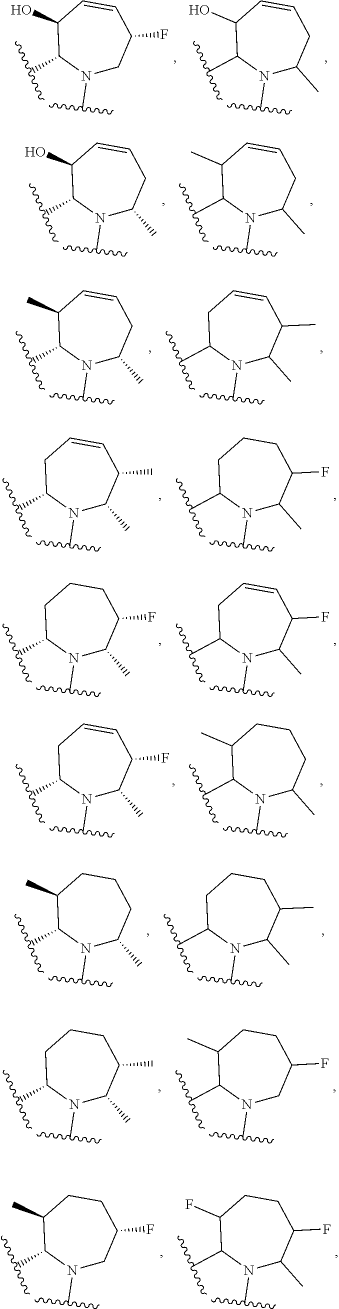

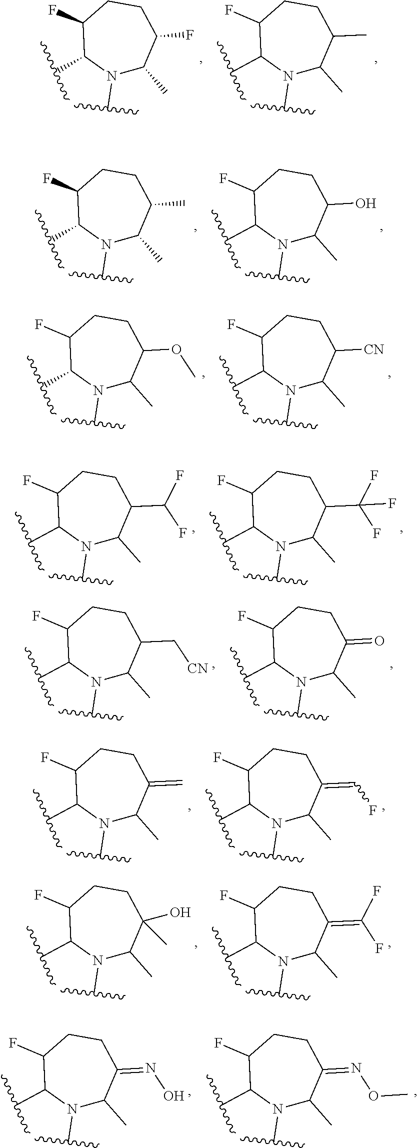

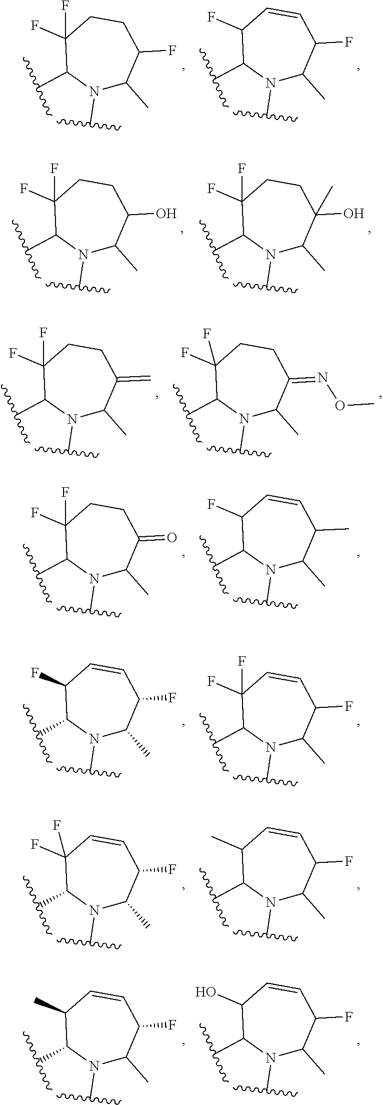

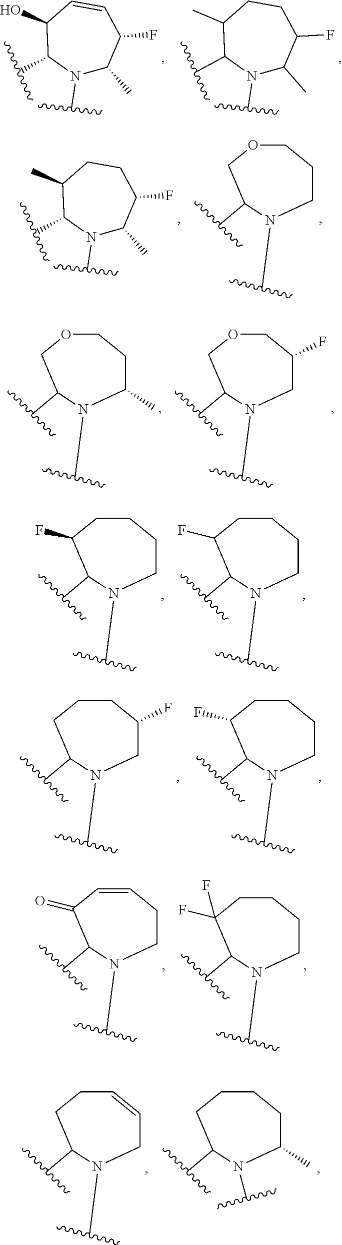

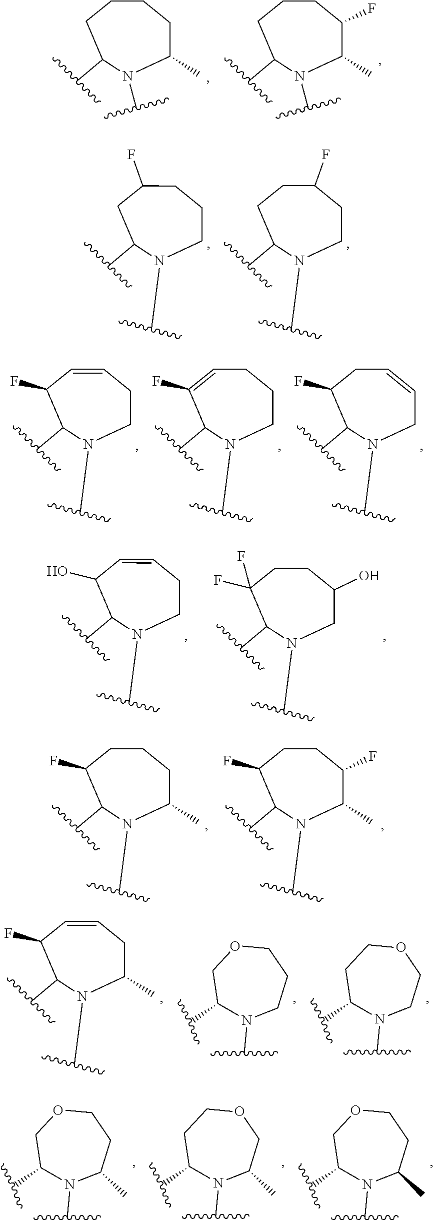

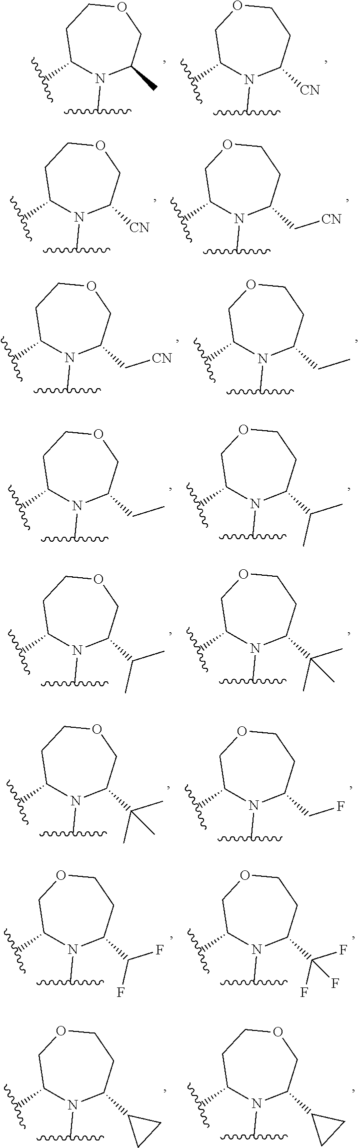

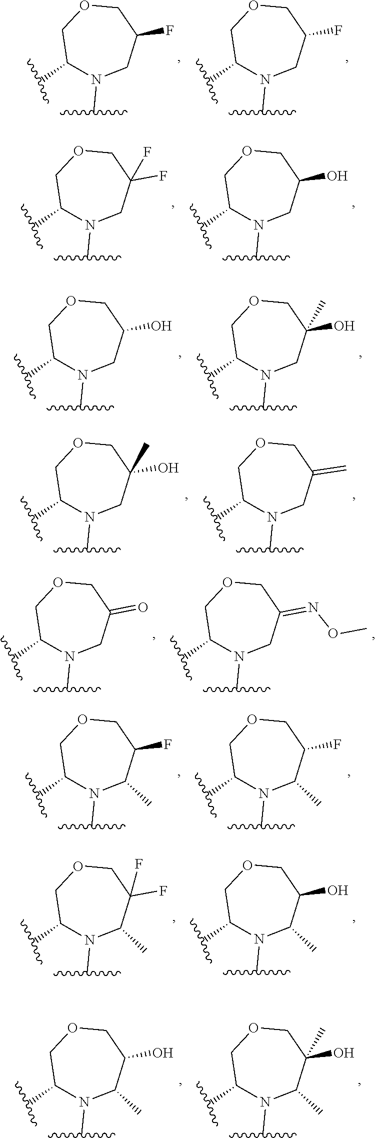

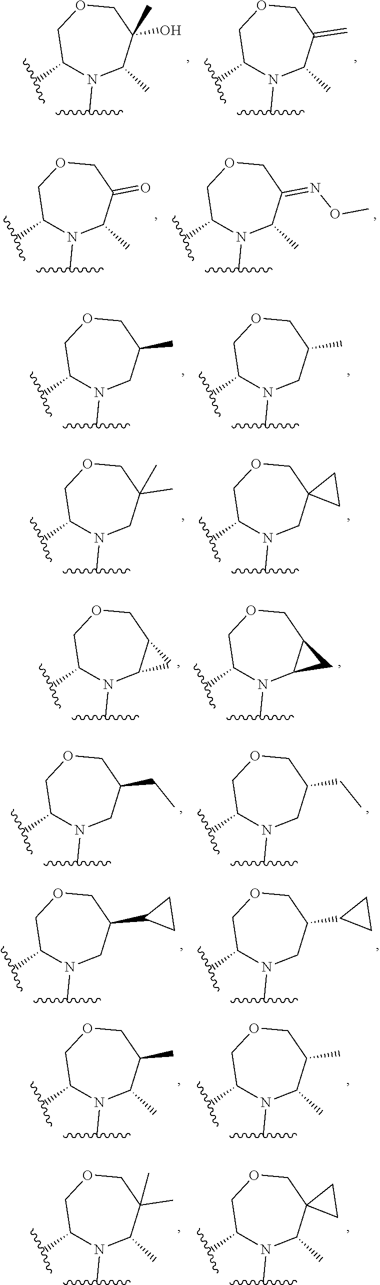

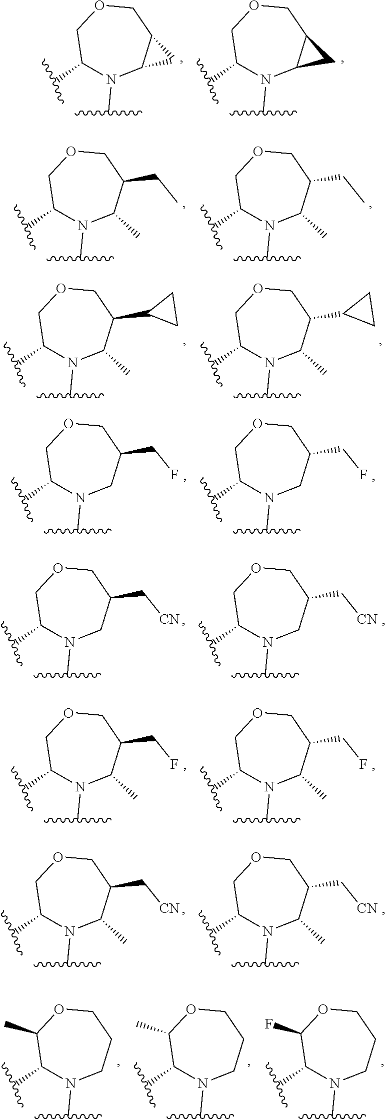

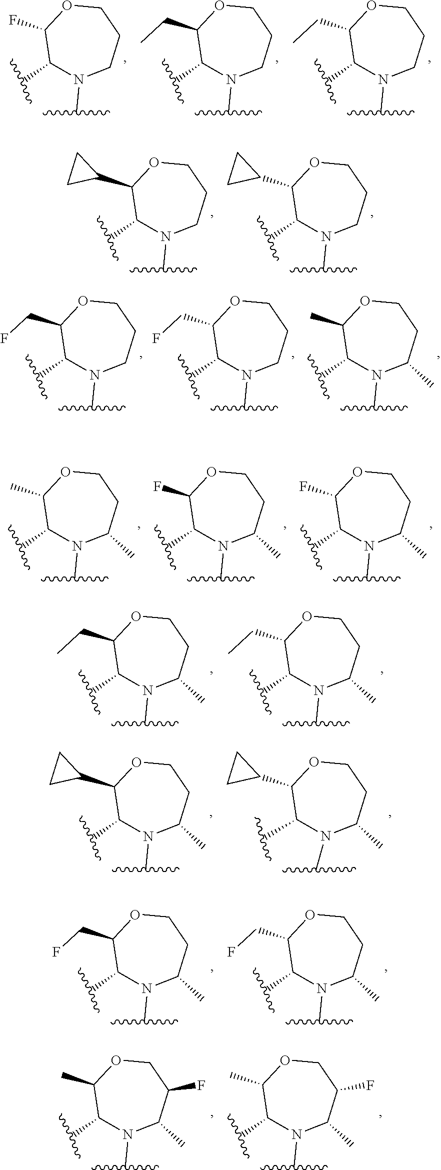

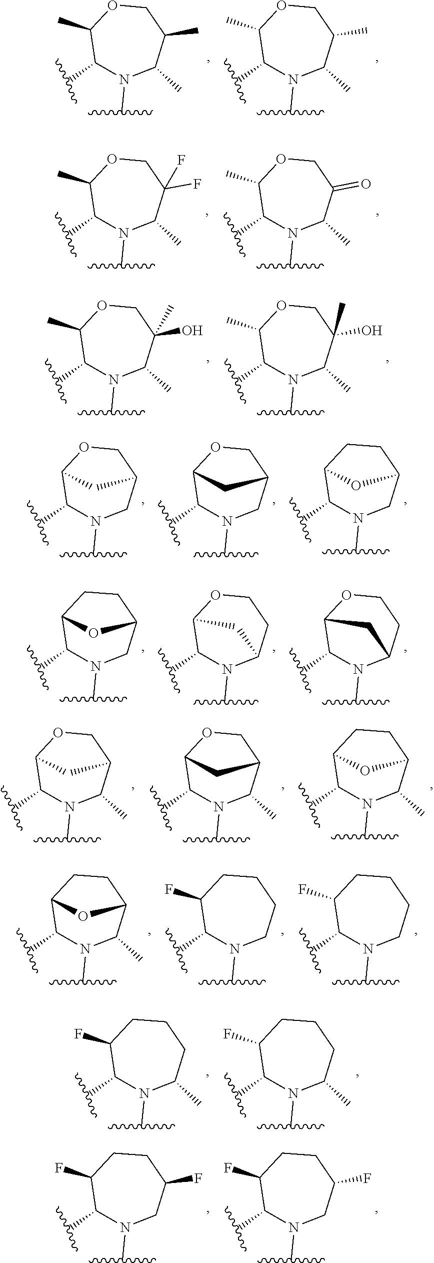

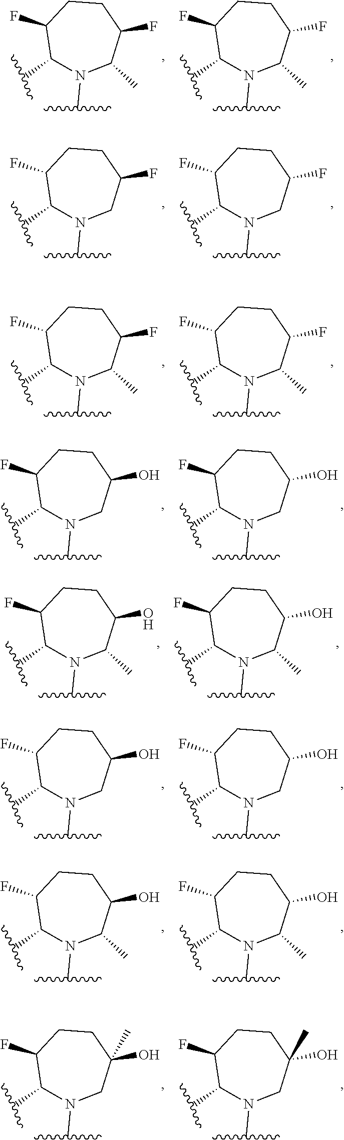

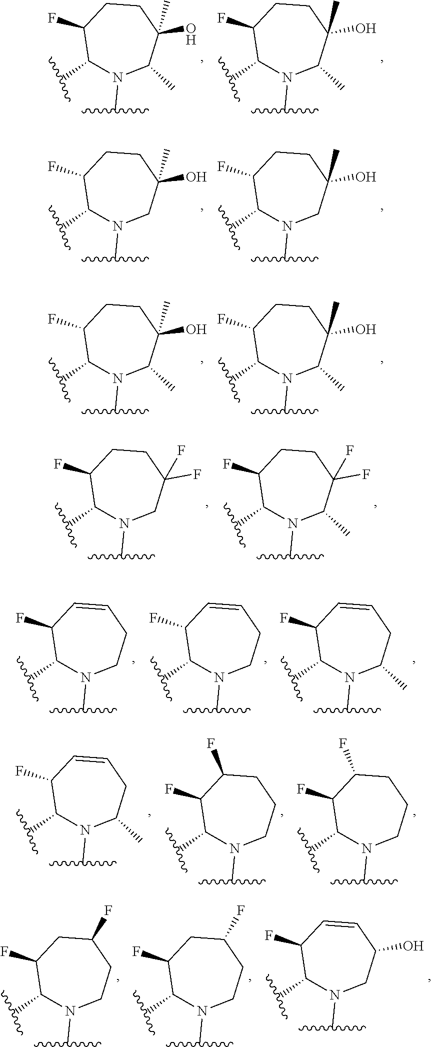

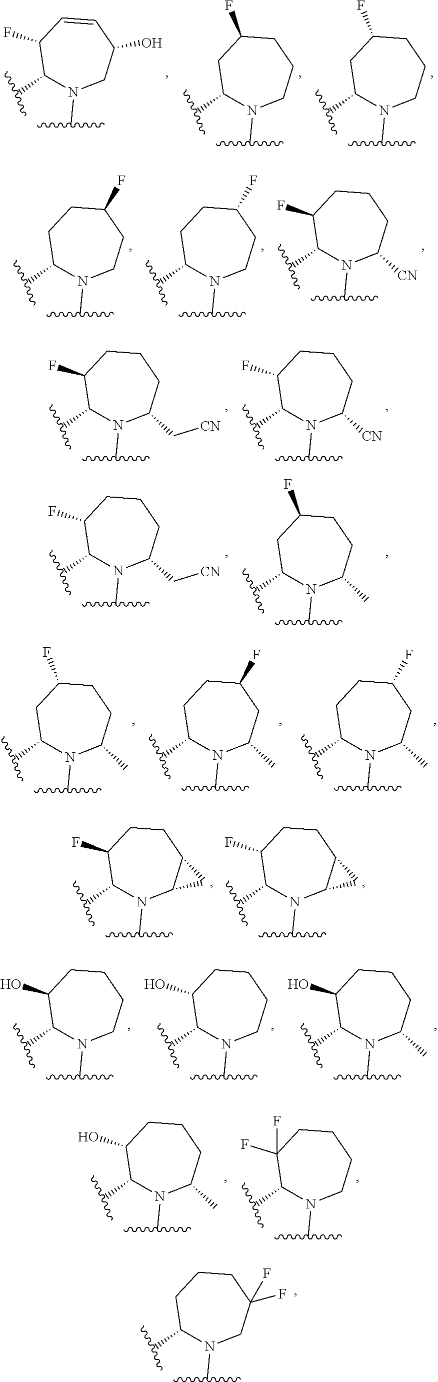

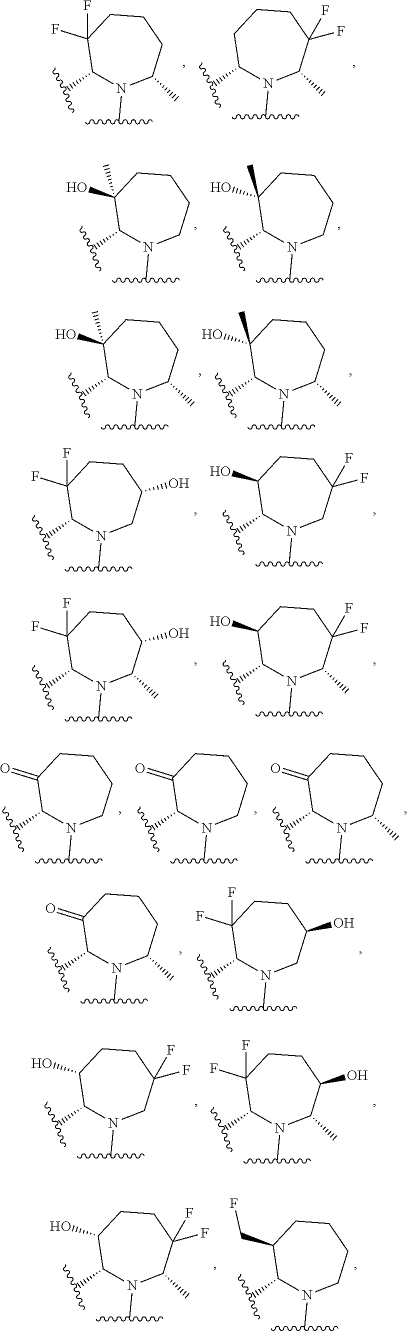

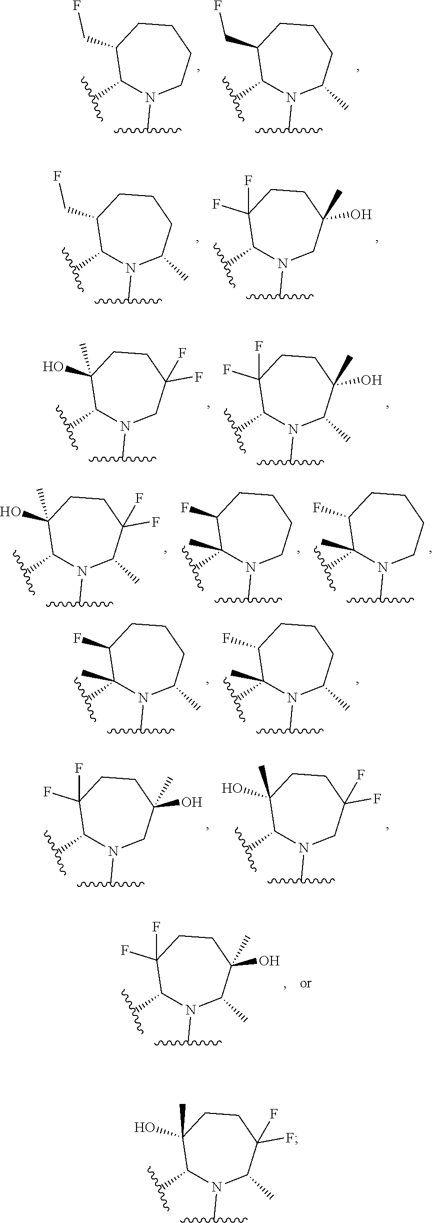

In some embodiments,

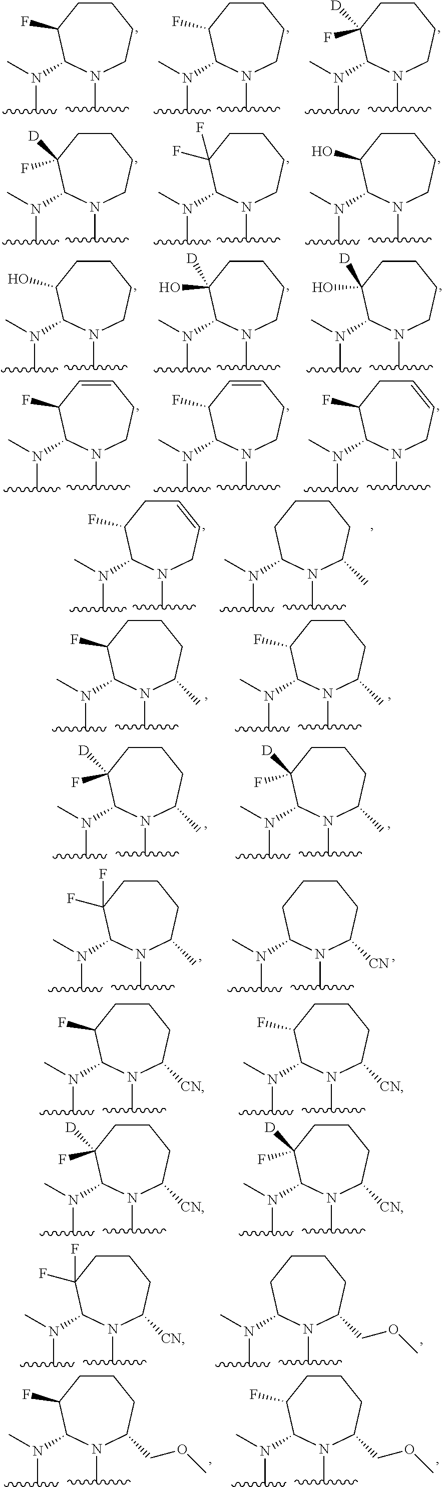

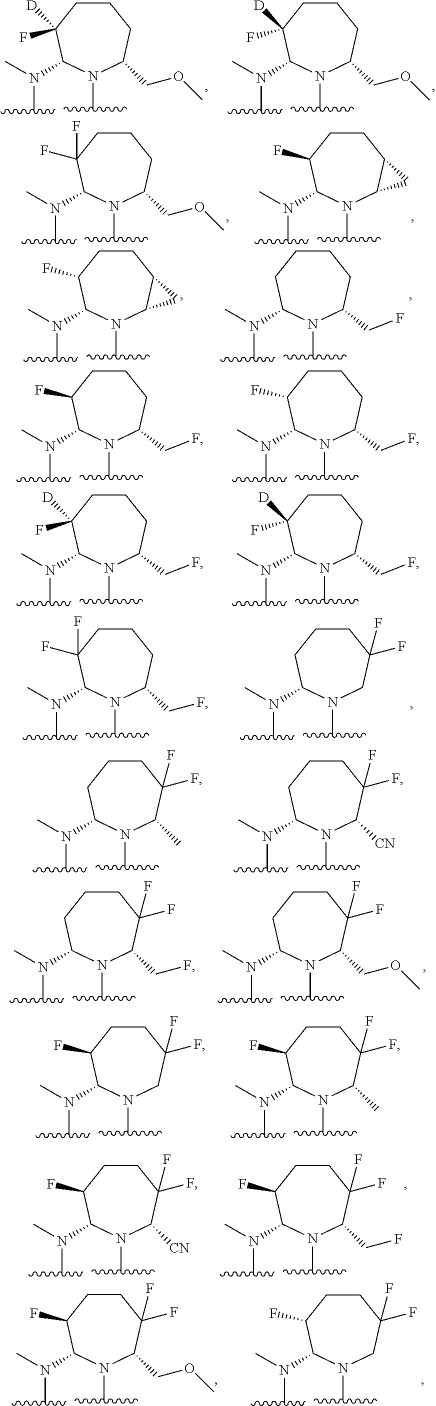

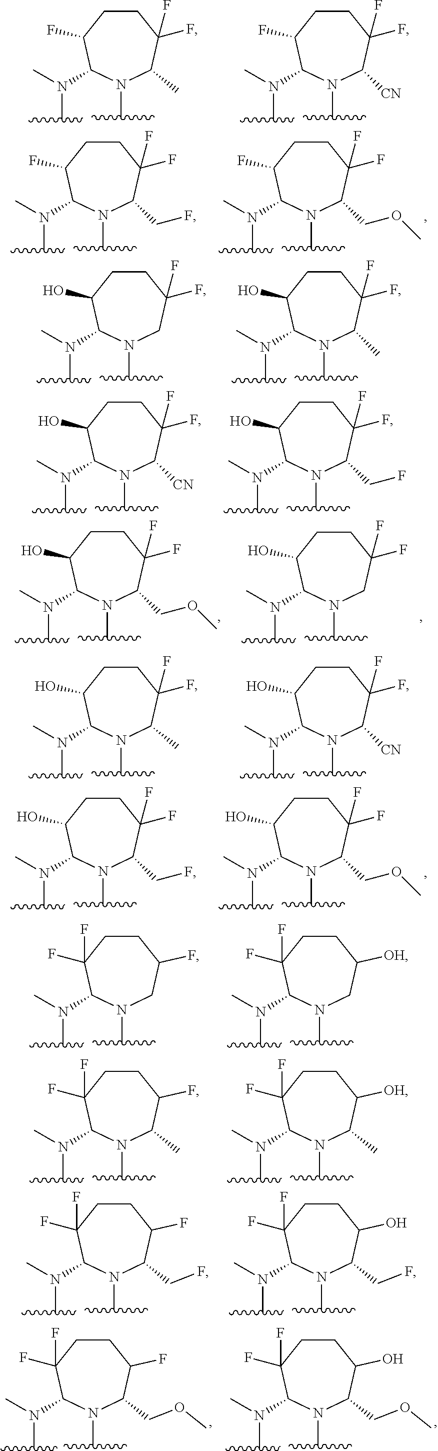

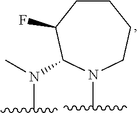 [SMILES: *N(C)[C@@H]1[C@@H](F)CCCCN1*]

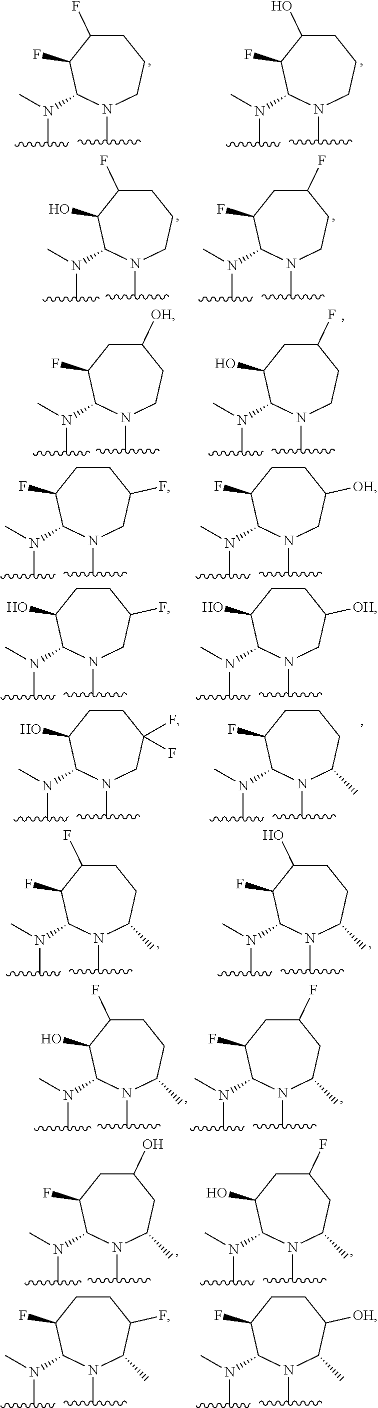

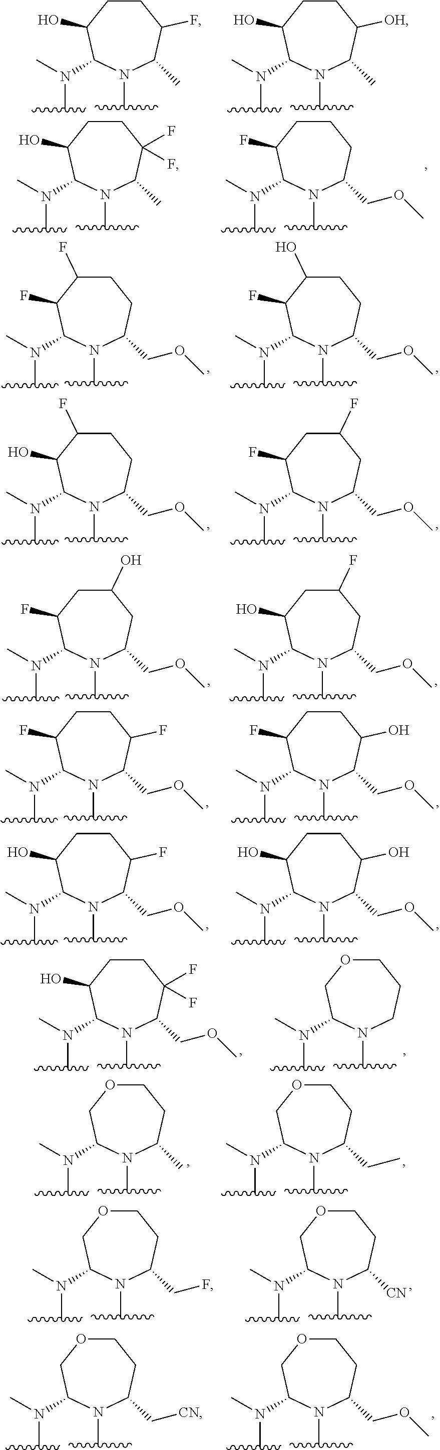

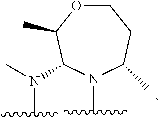

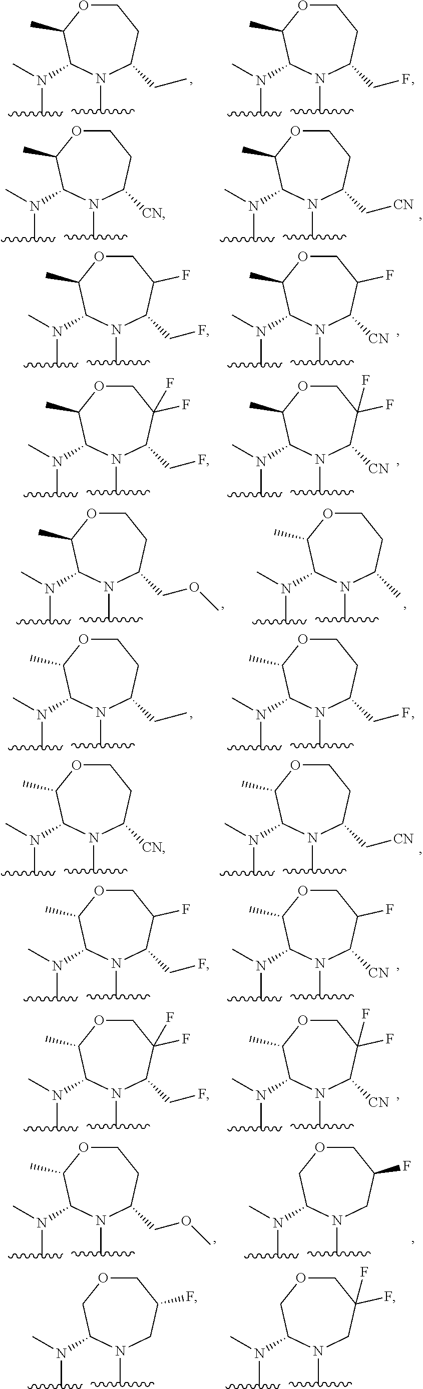

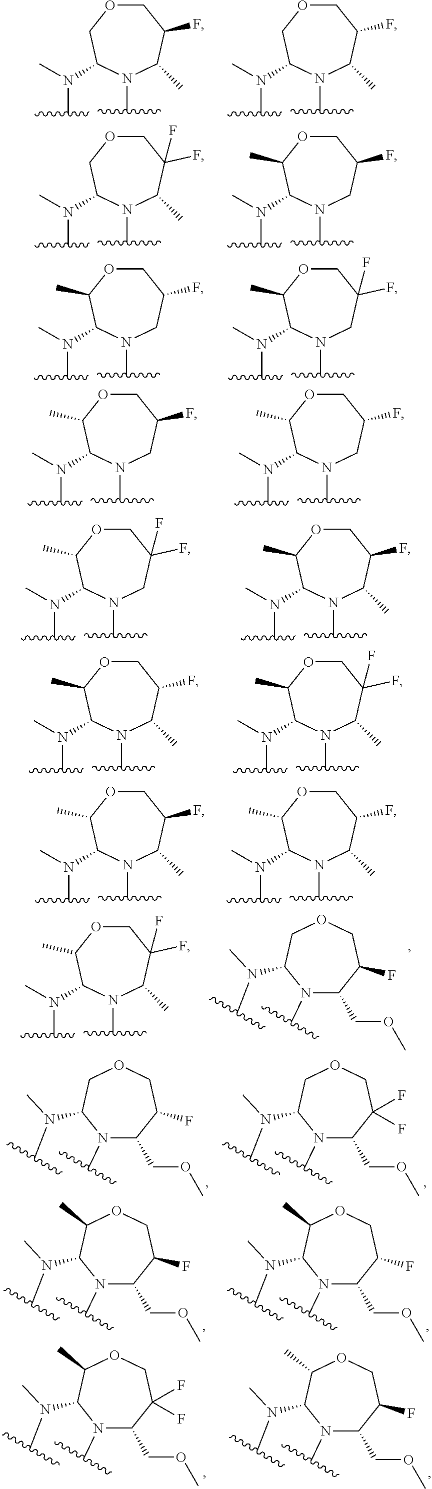

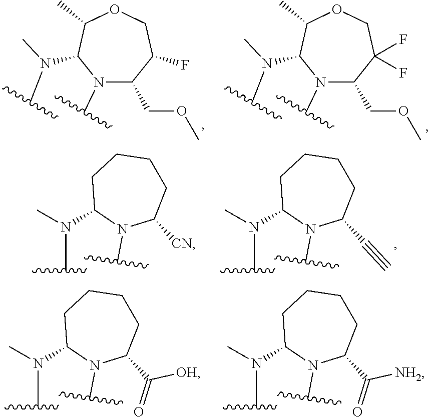

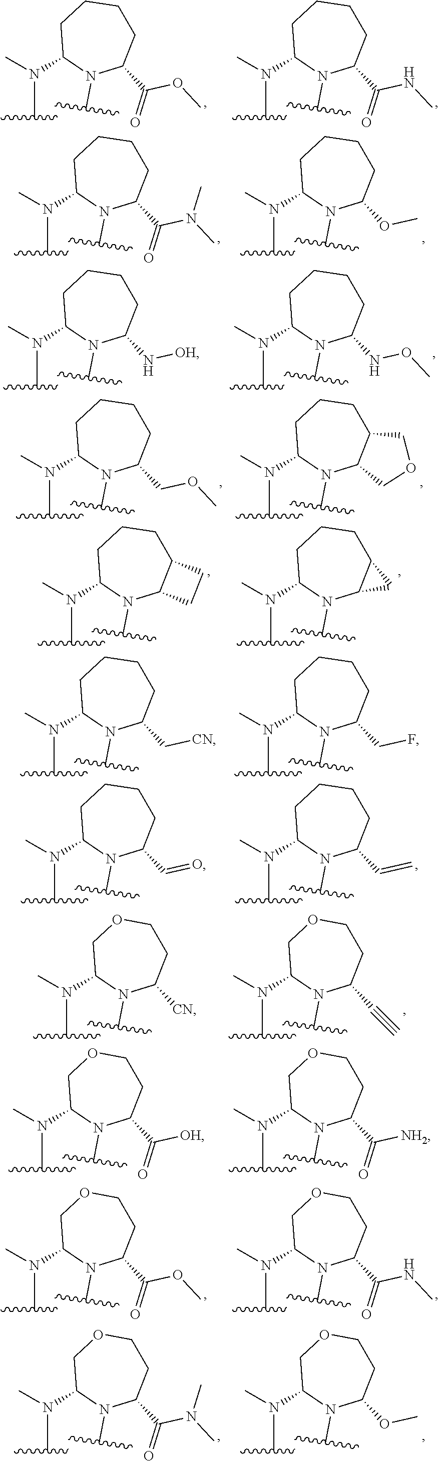

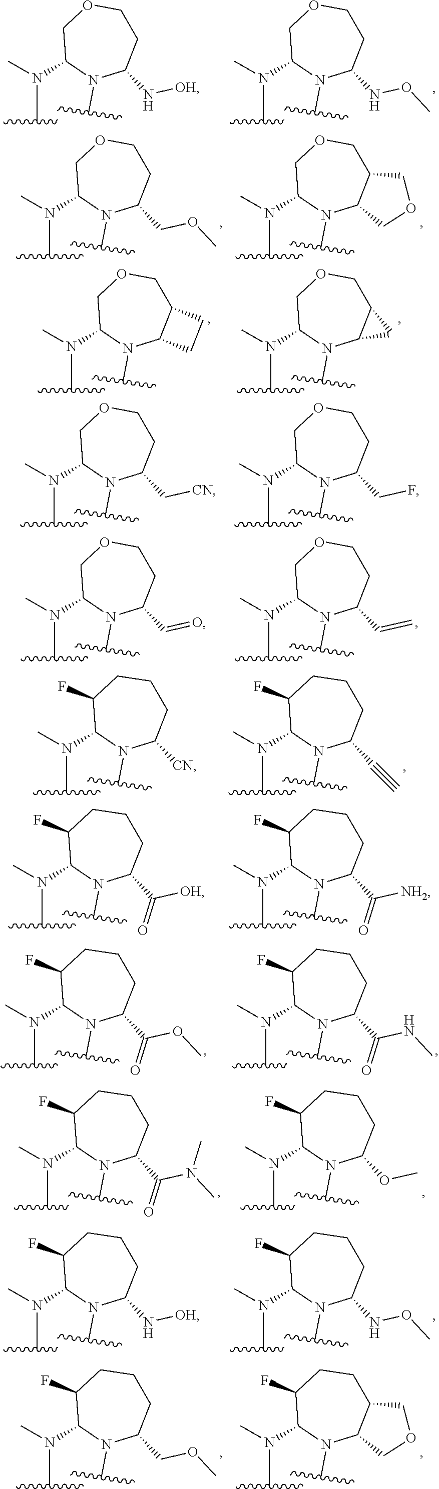

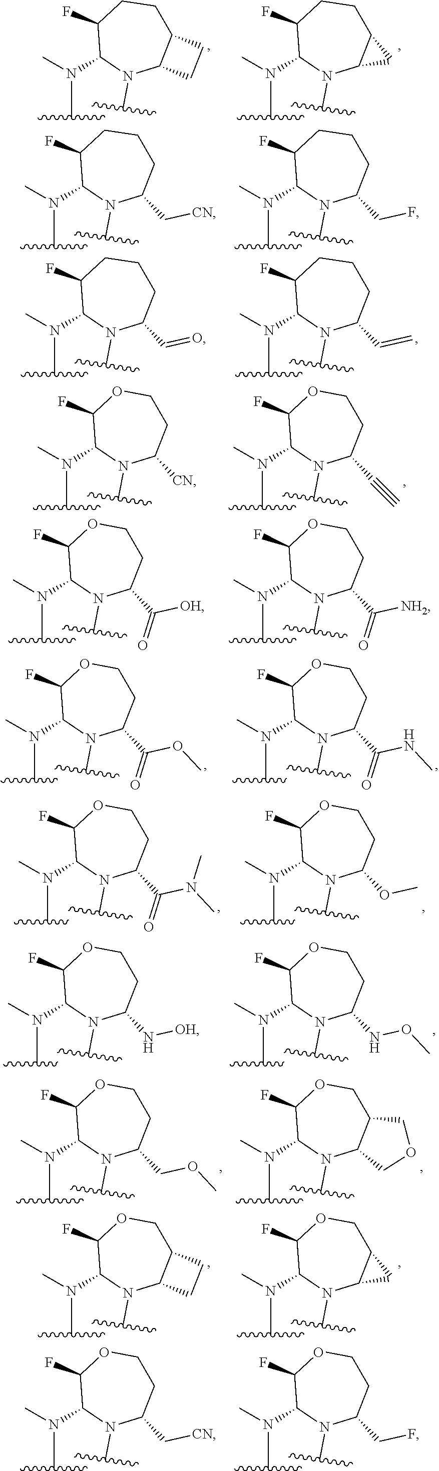

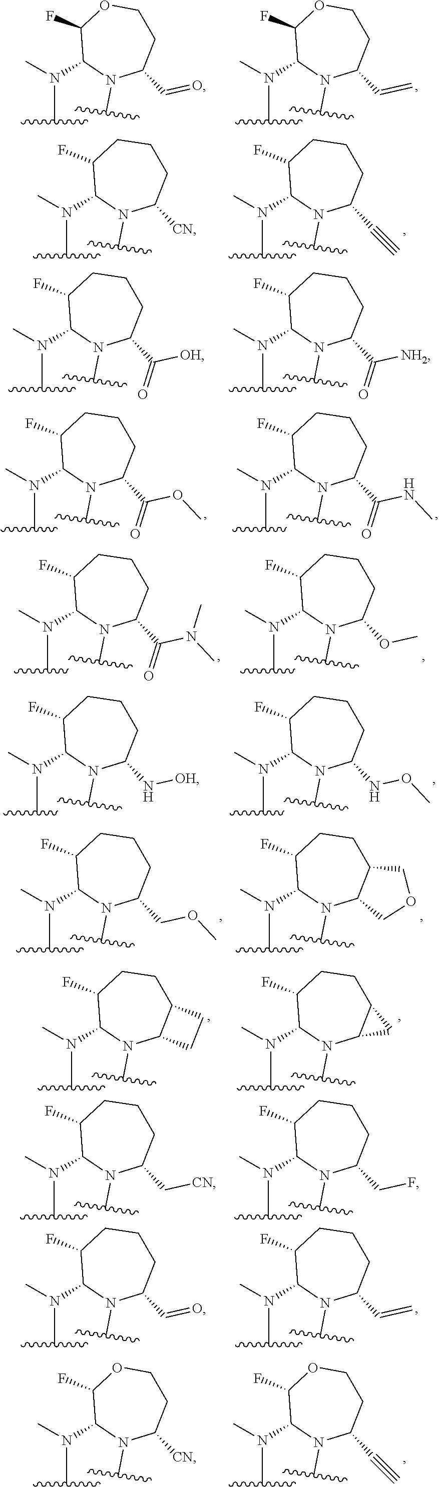

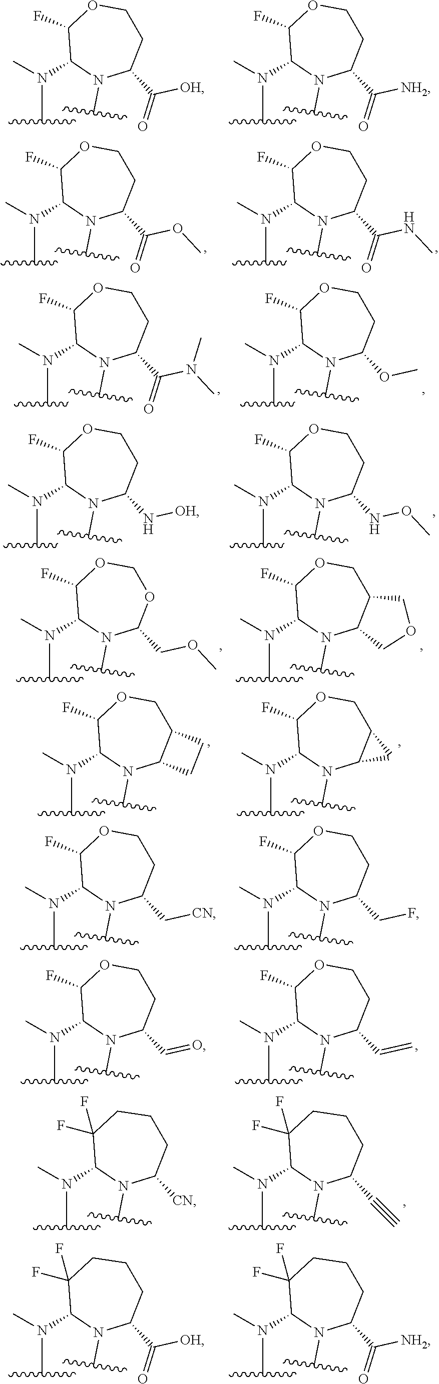

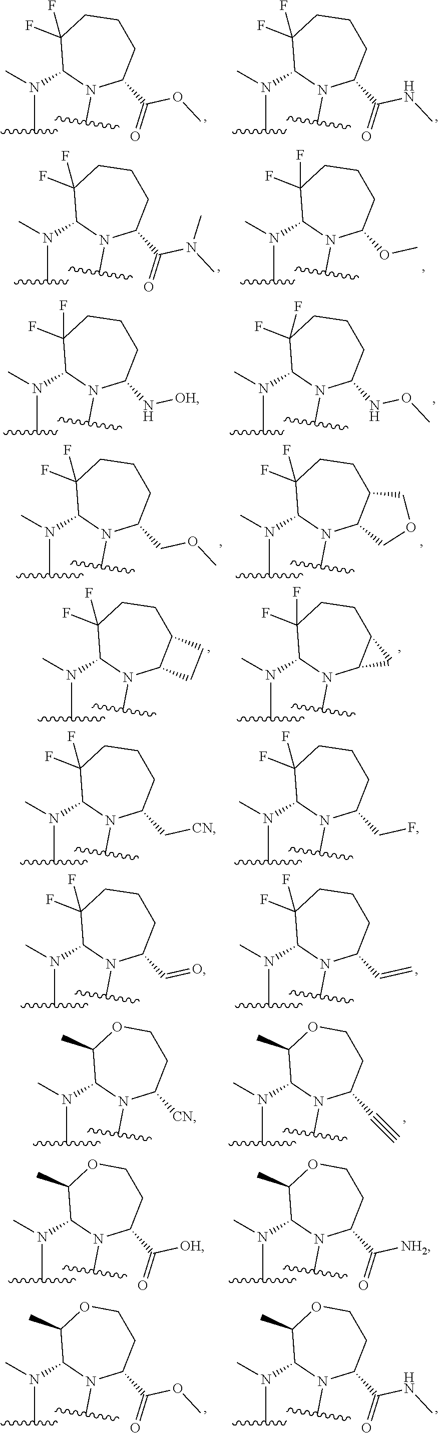

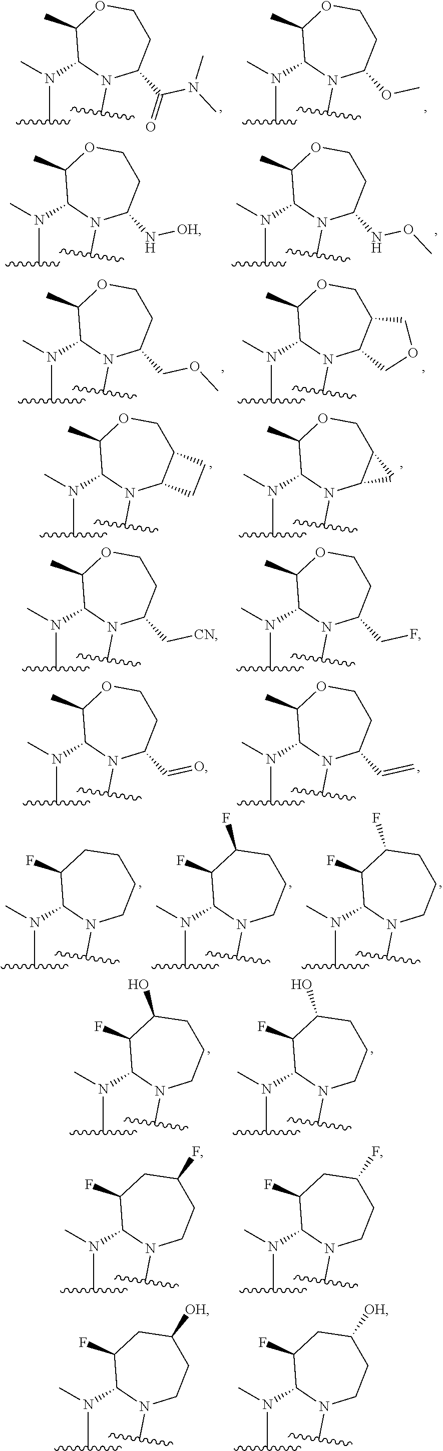

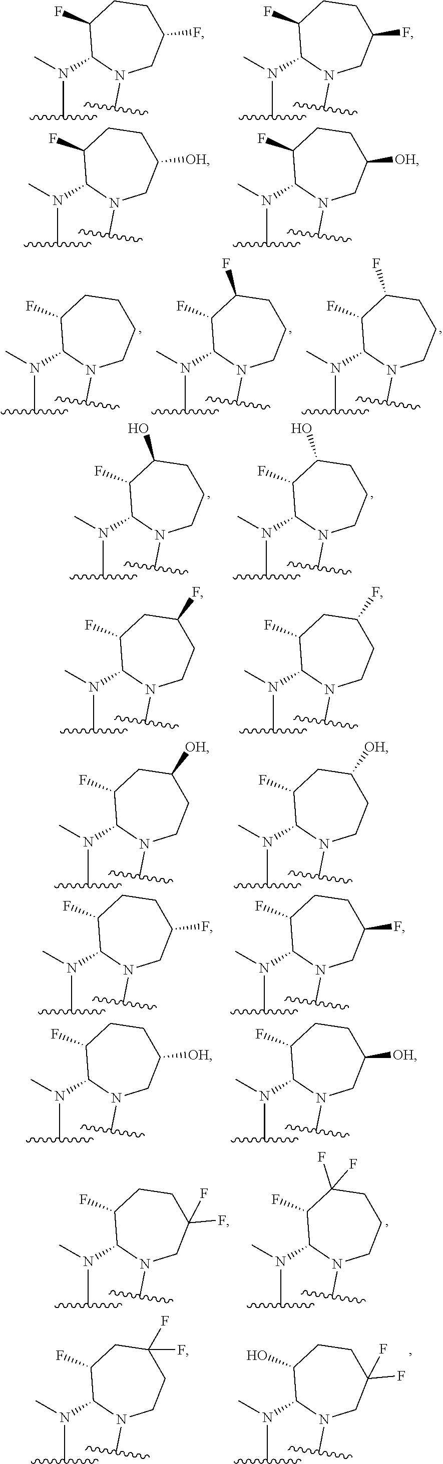

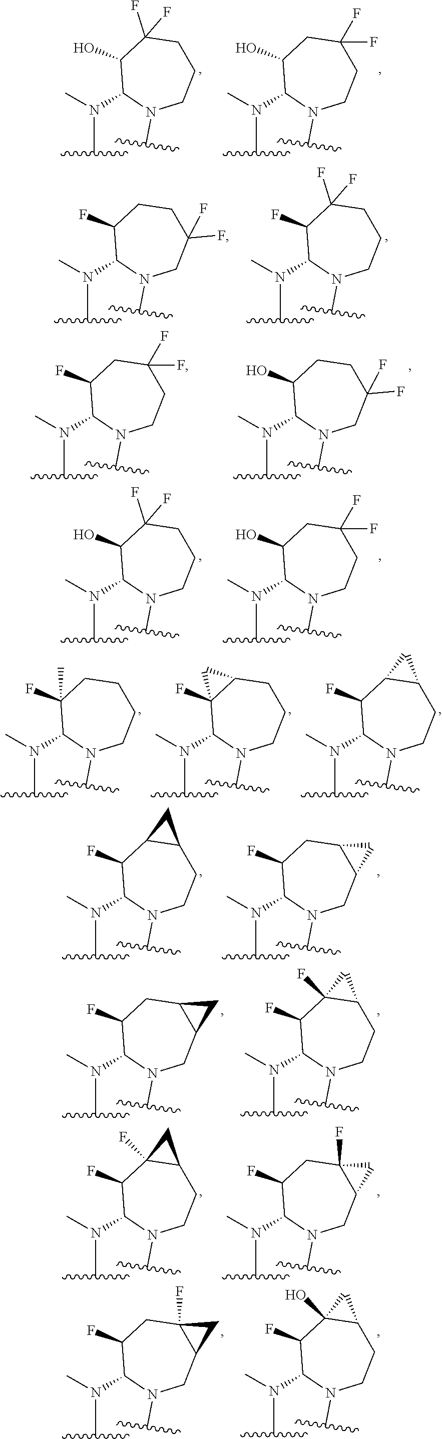

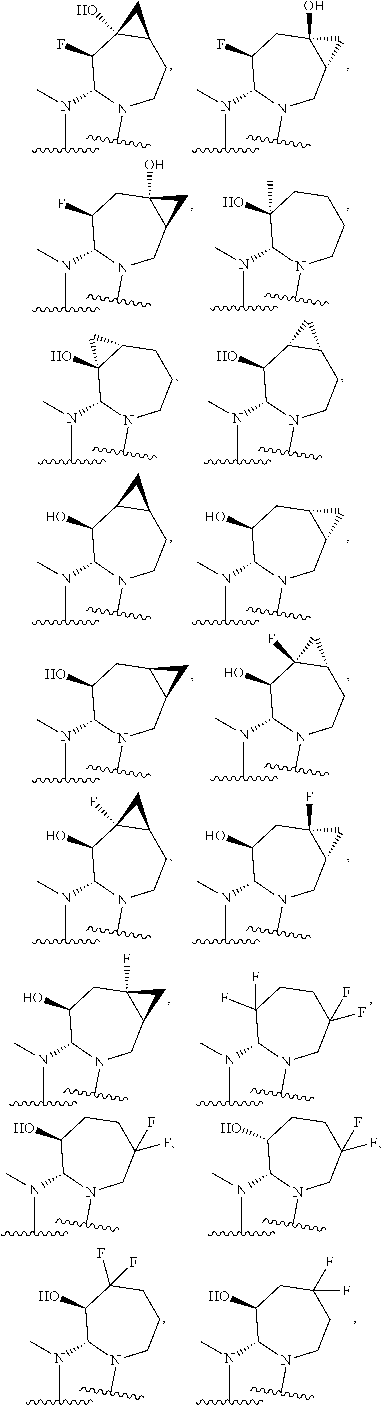

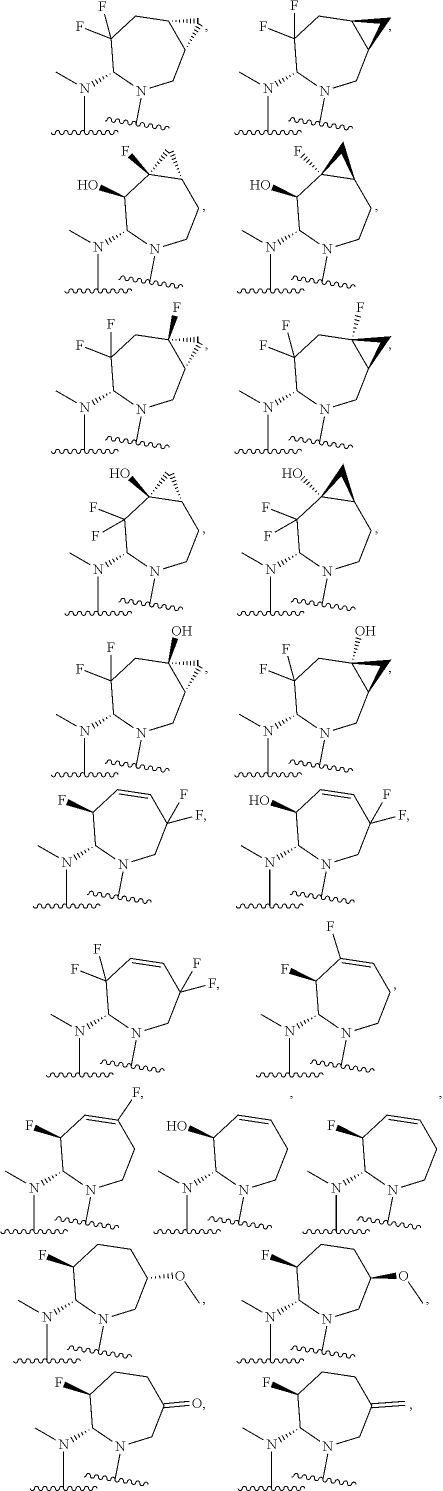

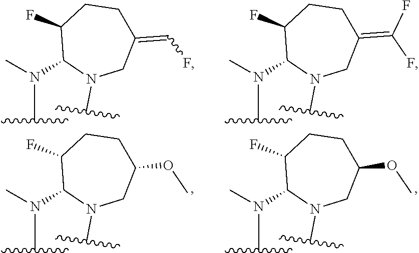

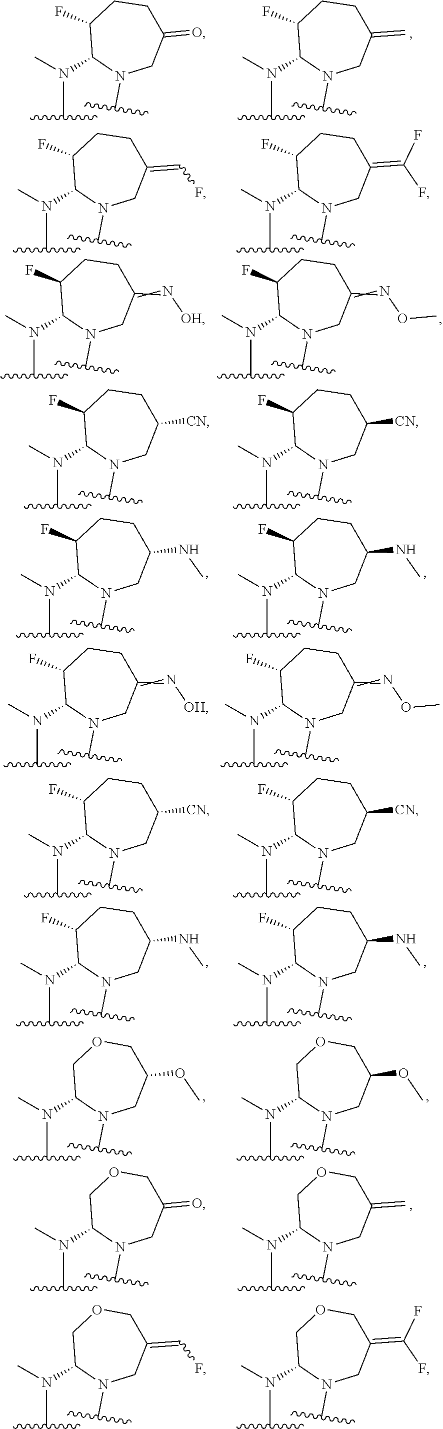

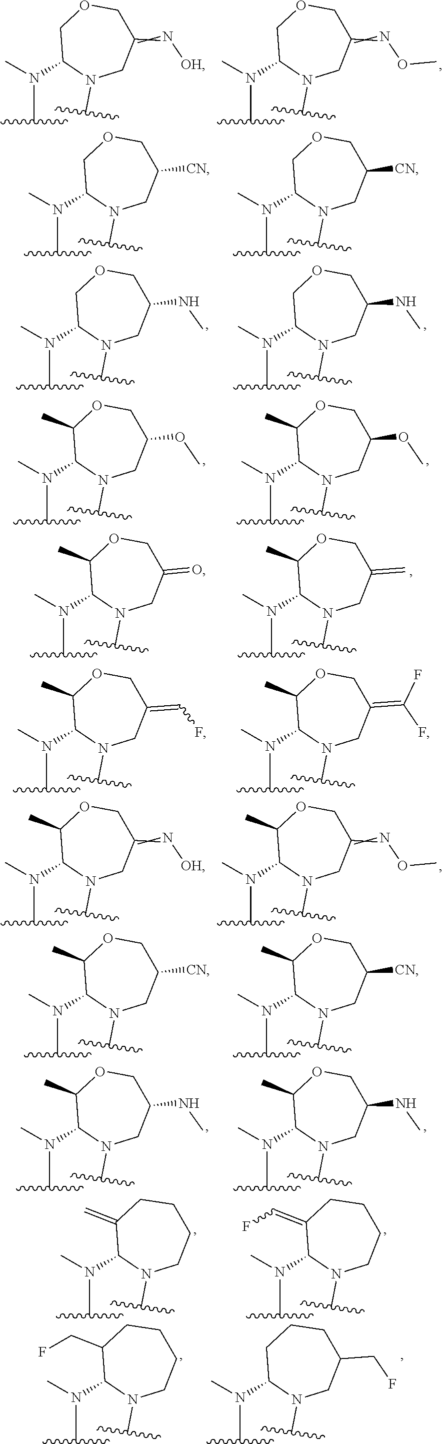

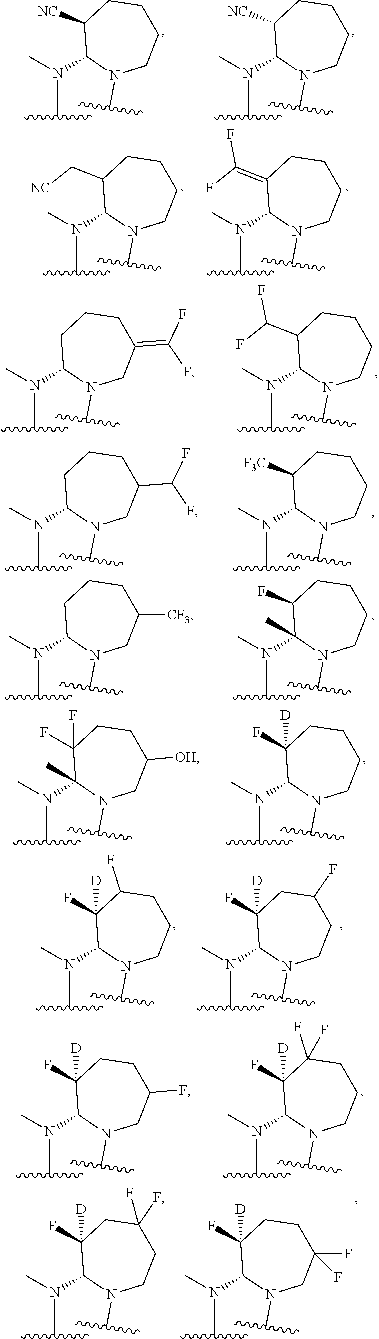

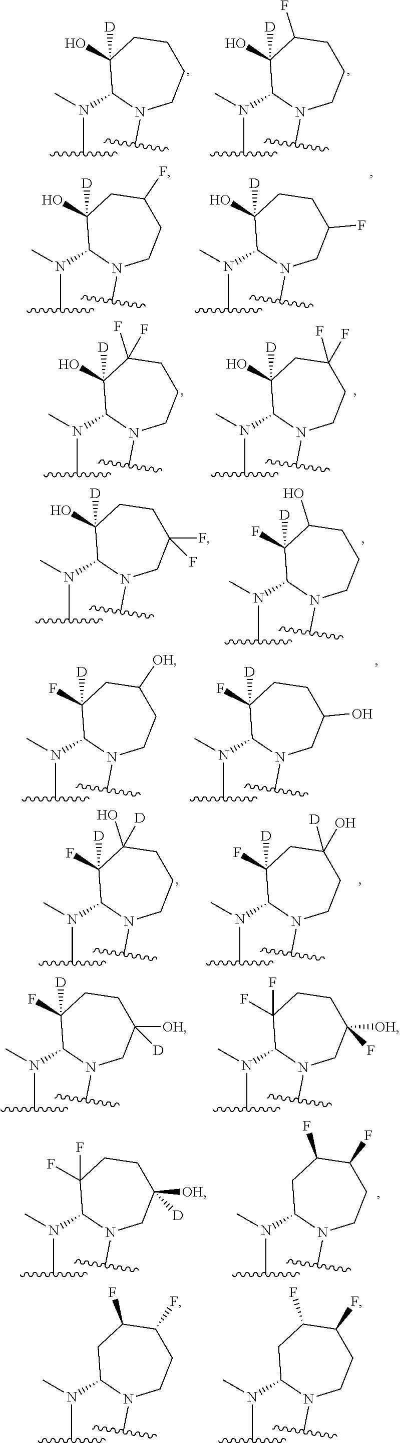

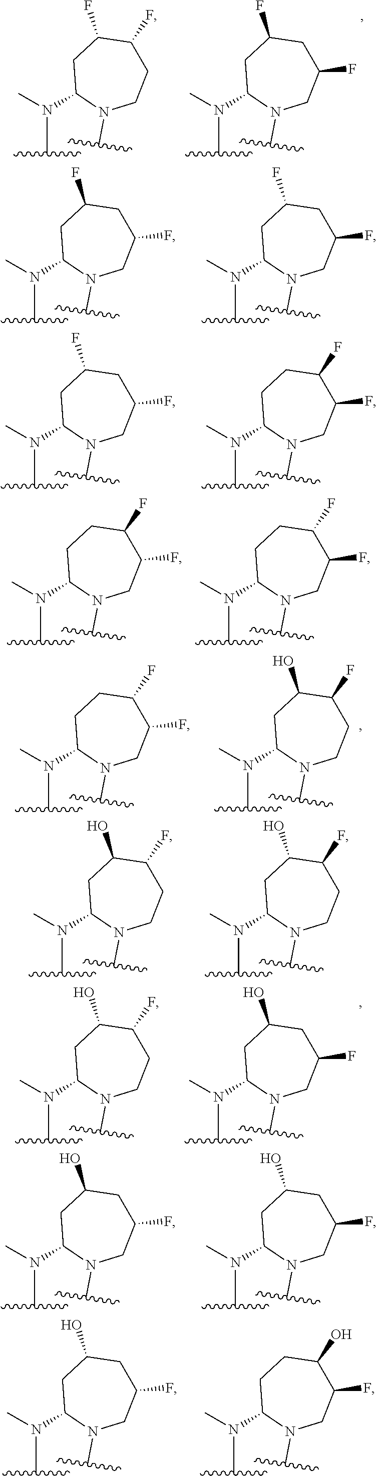

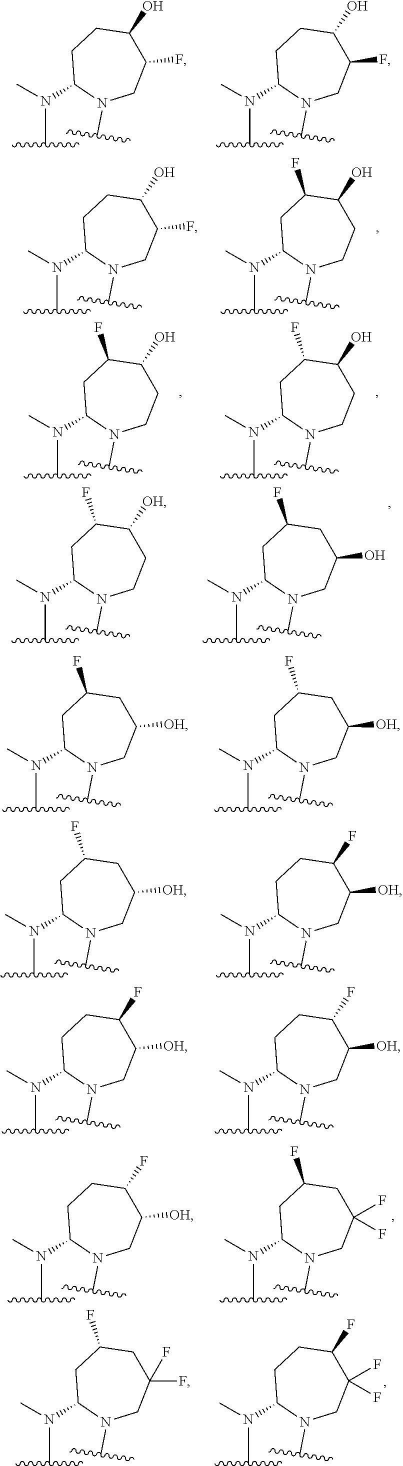

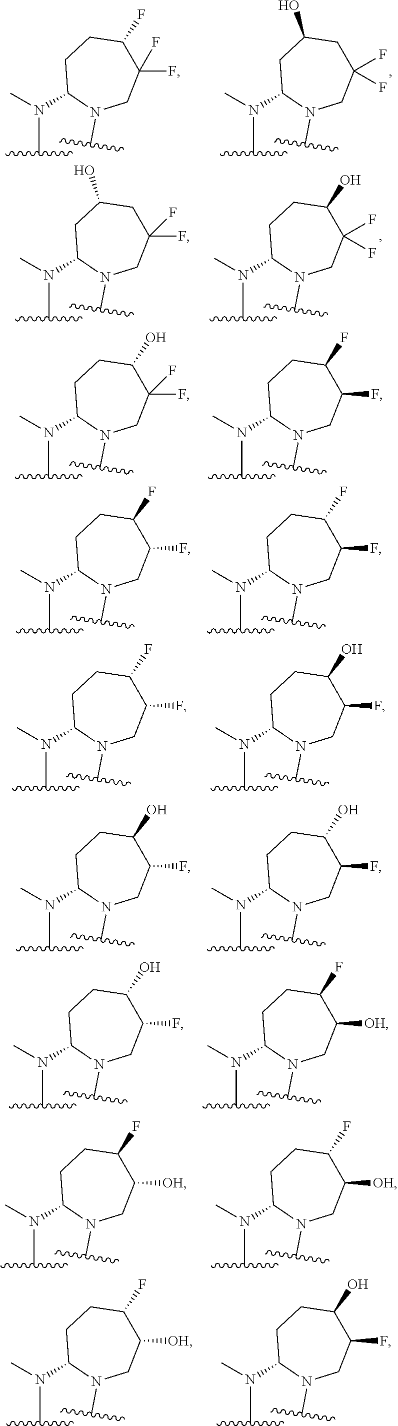

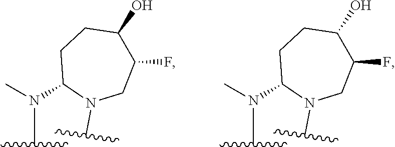 [SMILES: *N(C)[C@@H]1CC[C@@H](O)[C@H](F)CN1* ; *N(C)[C@@H]1CC[C@H](O)[C@@H](F)CN1*]

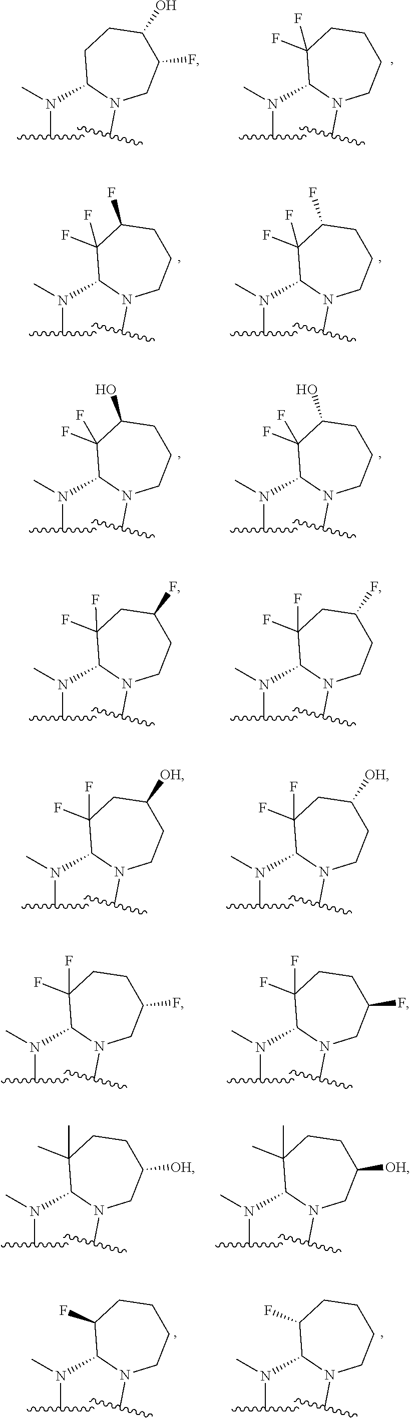

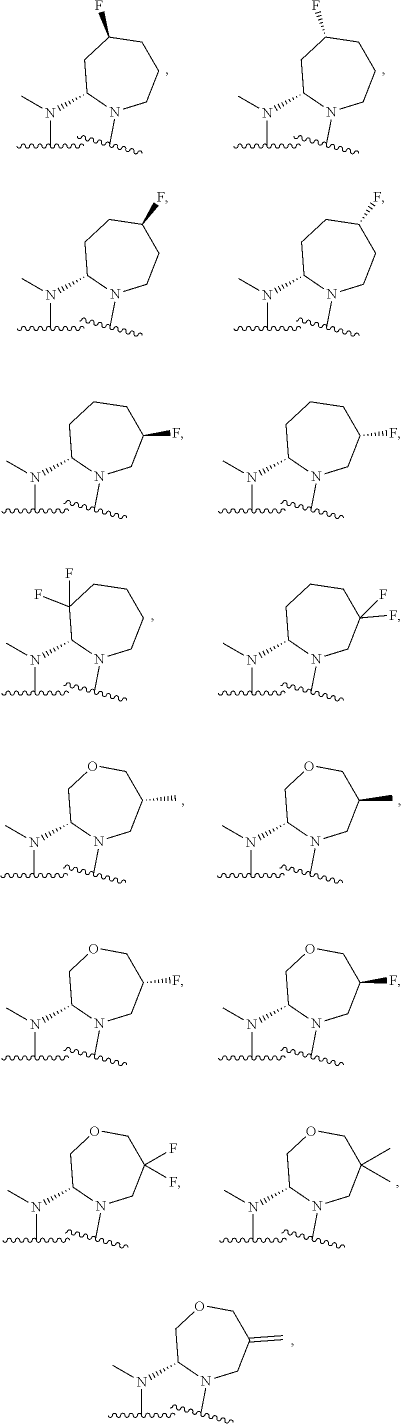

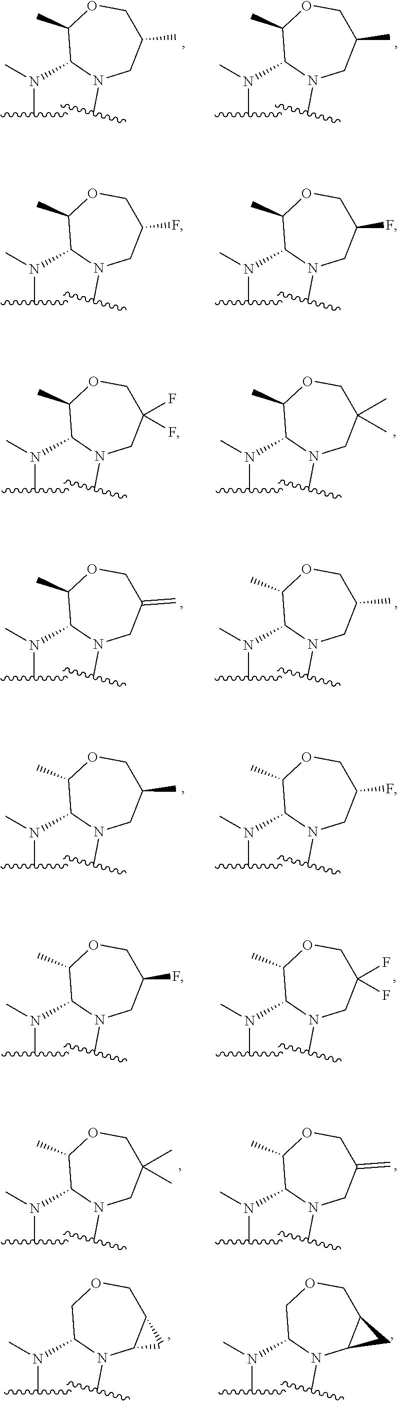

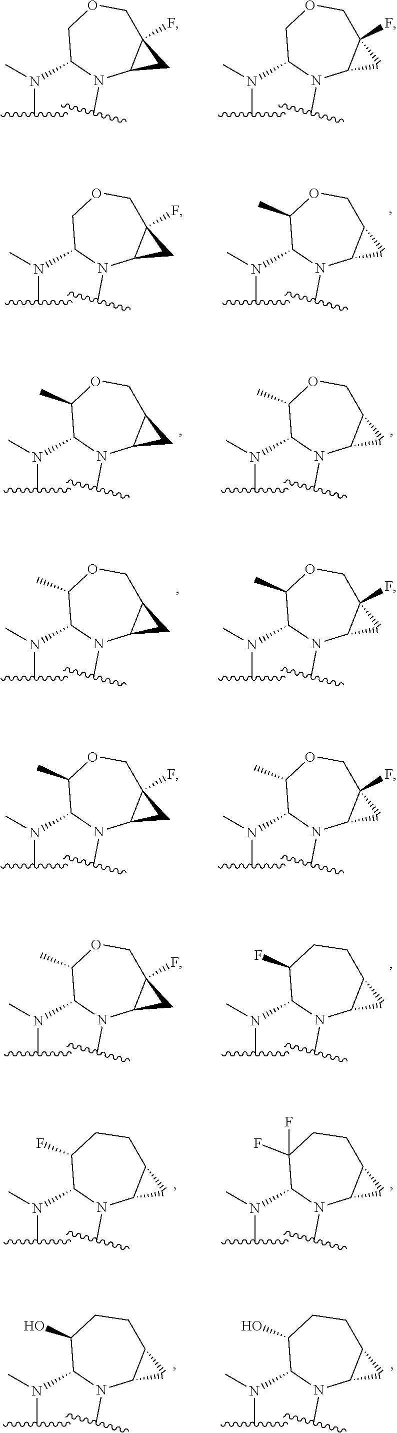

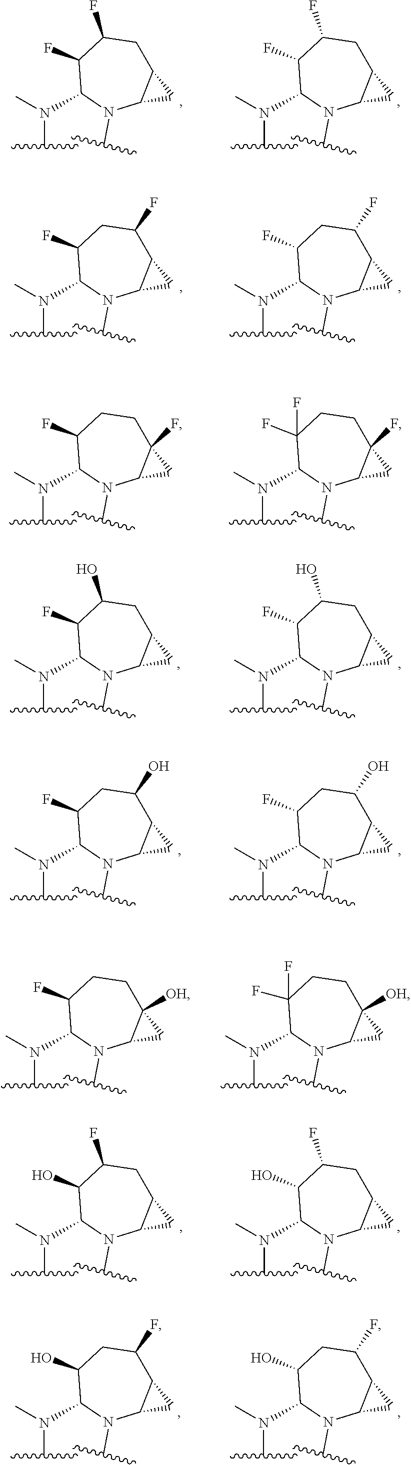

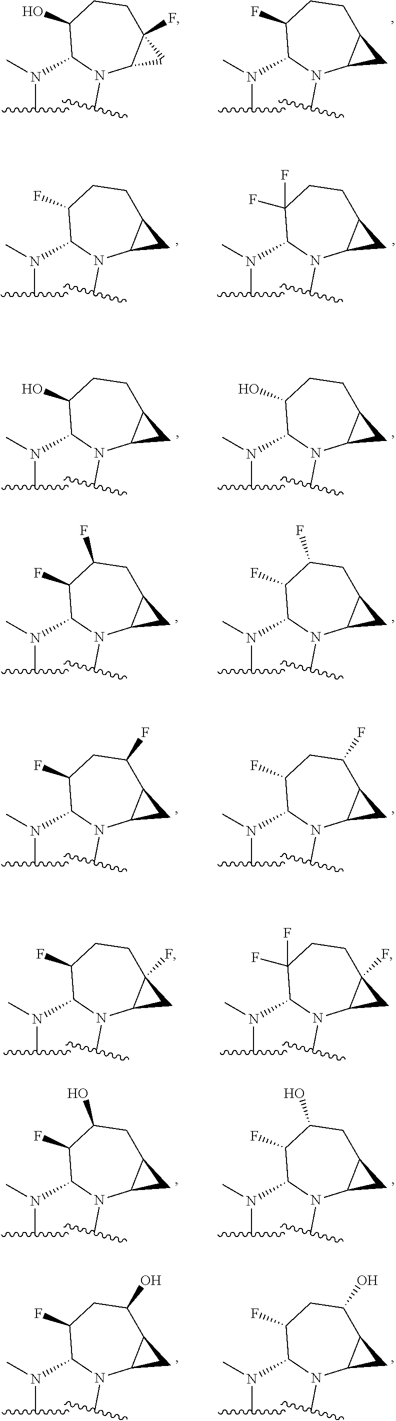

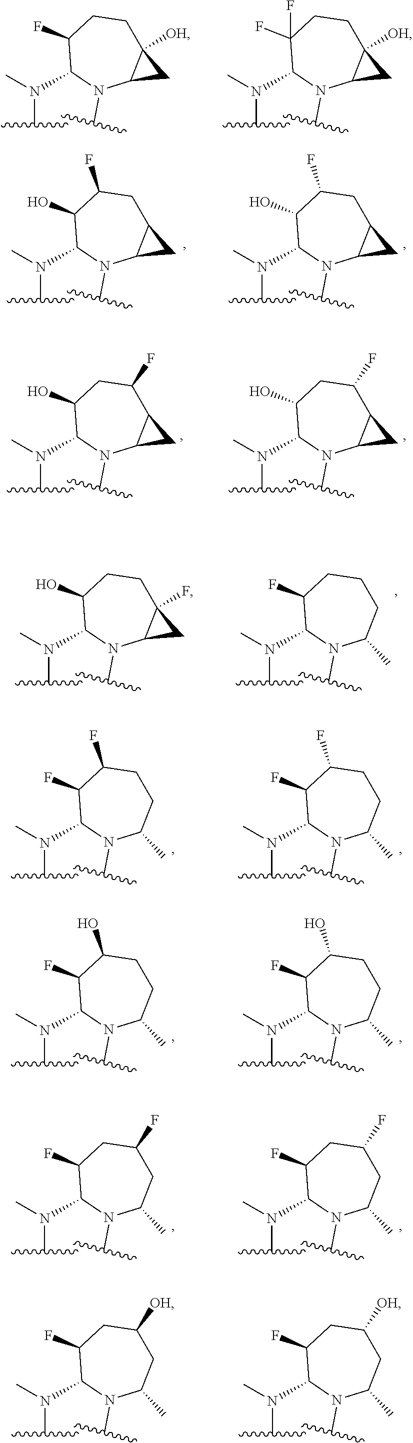

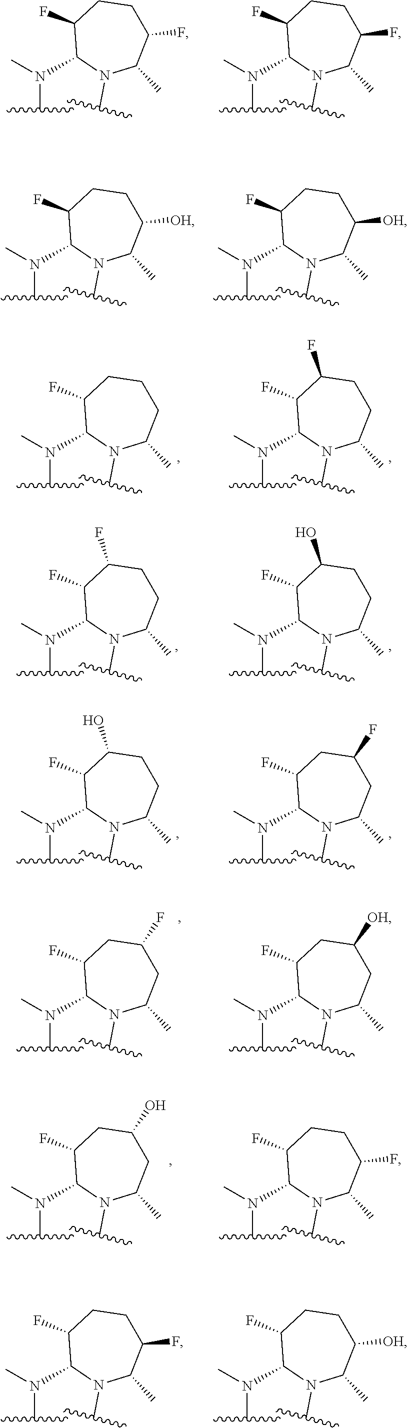

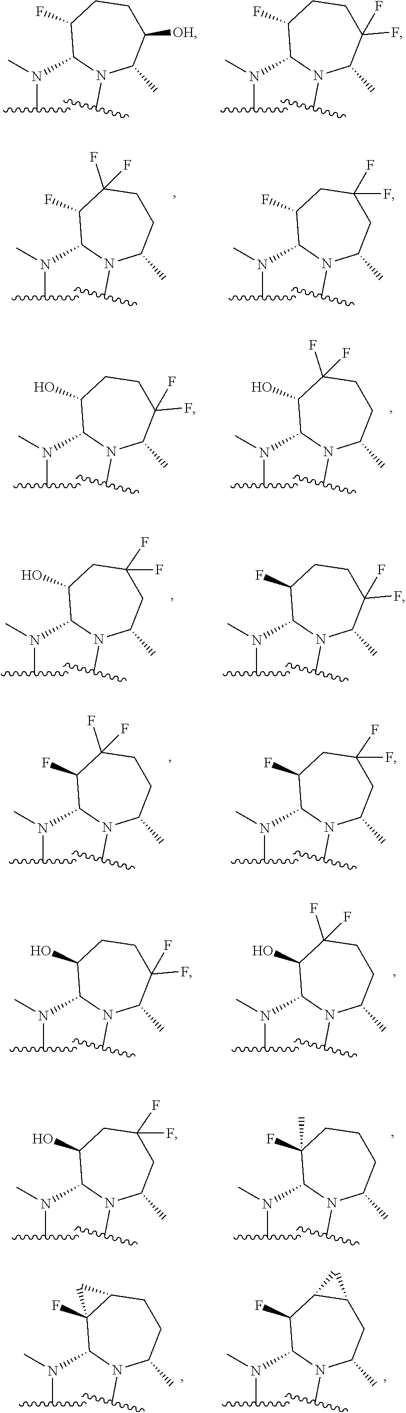

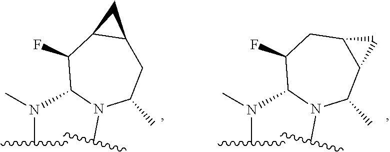

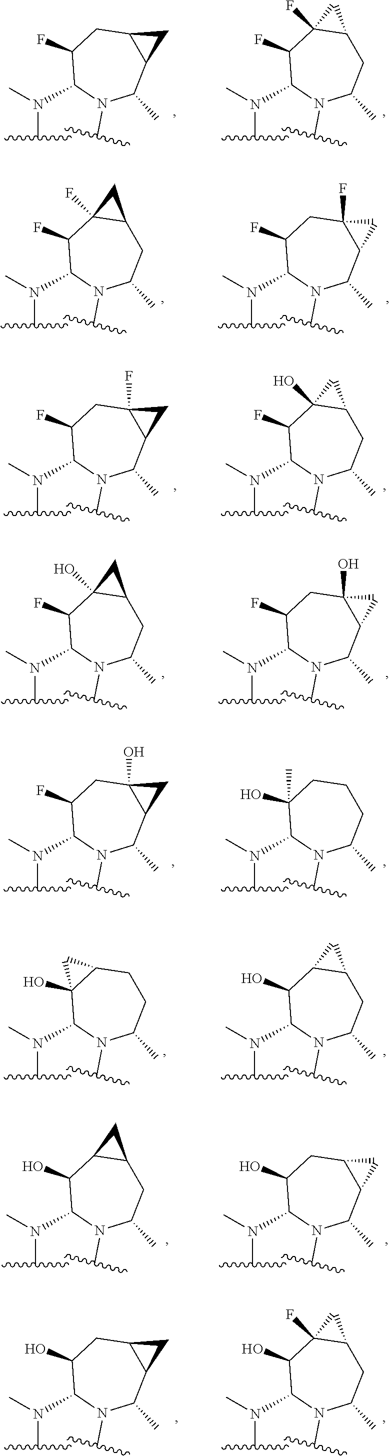

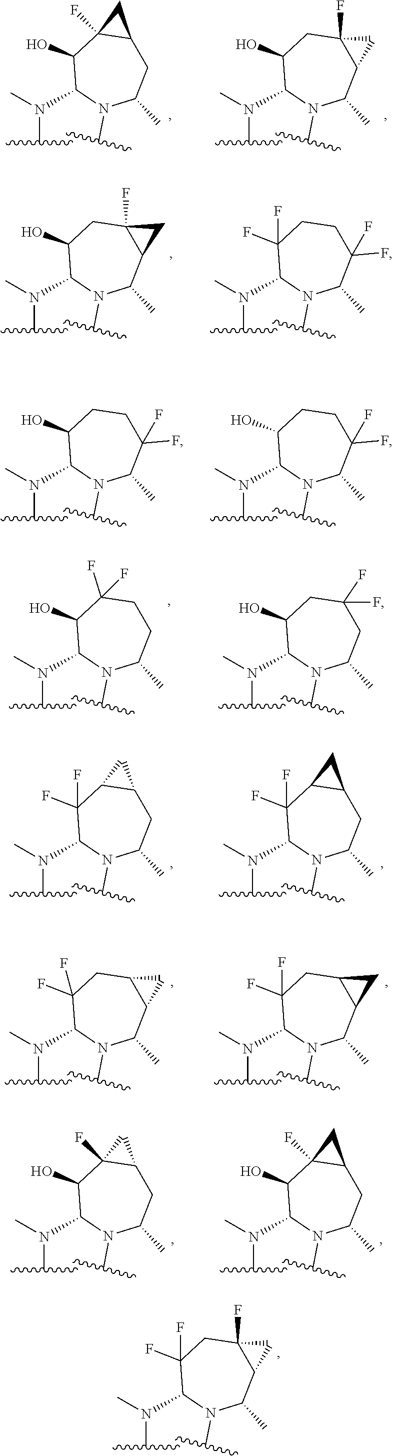

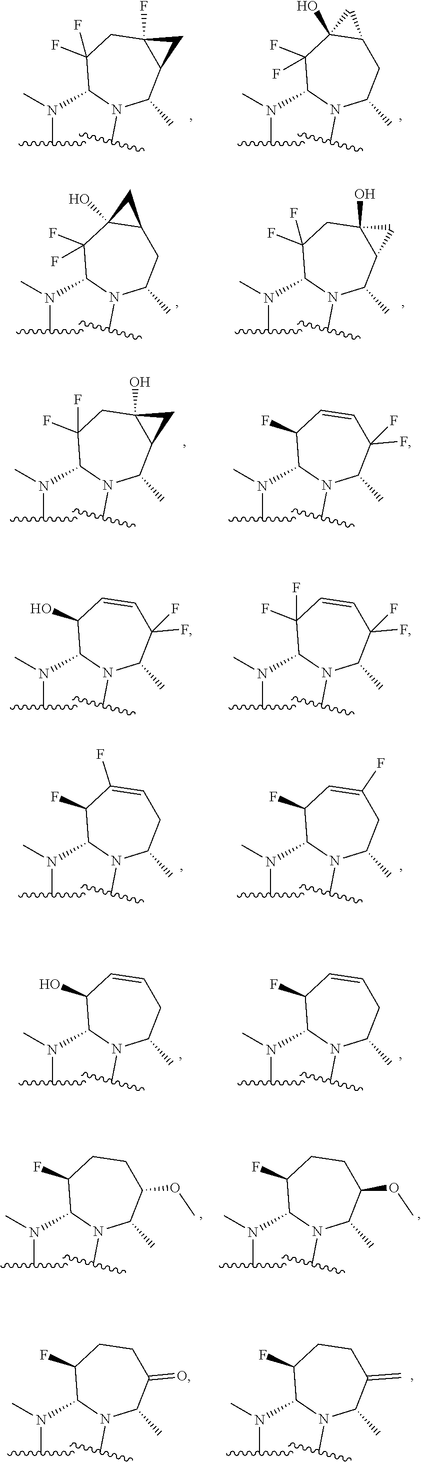

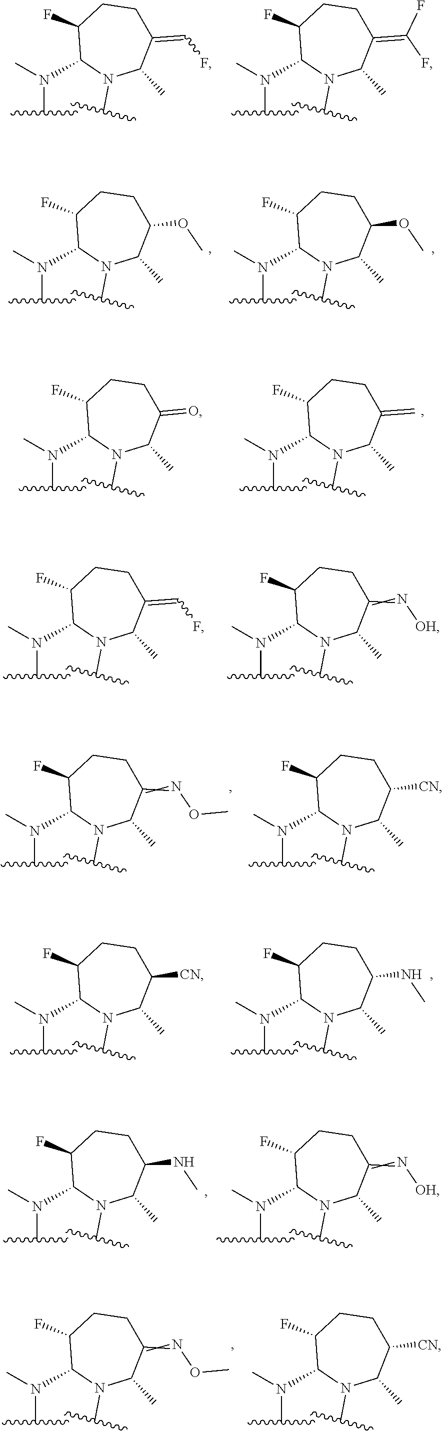

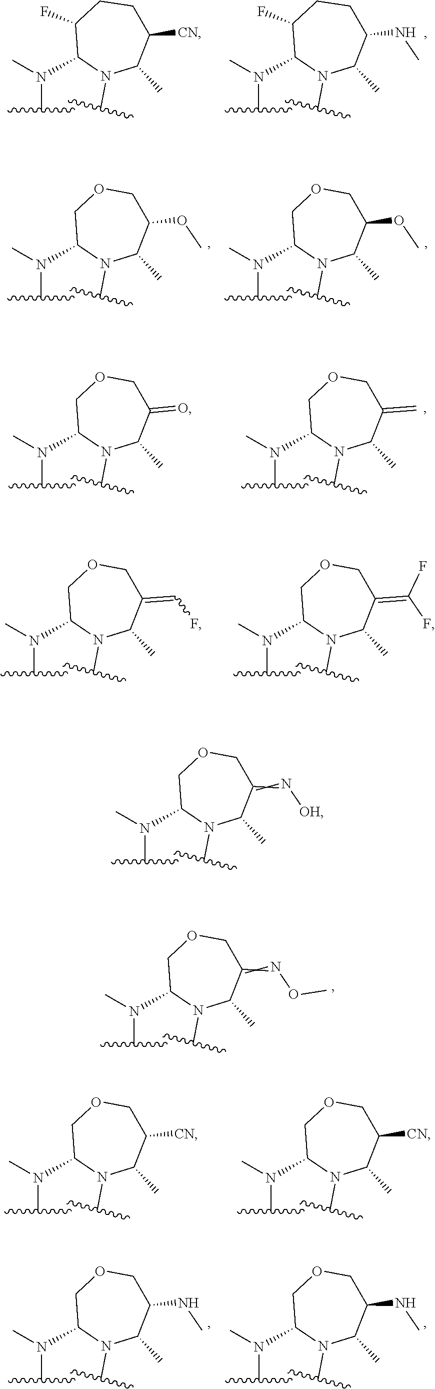

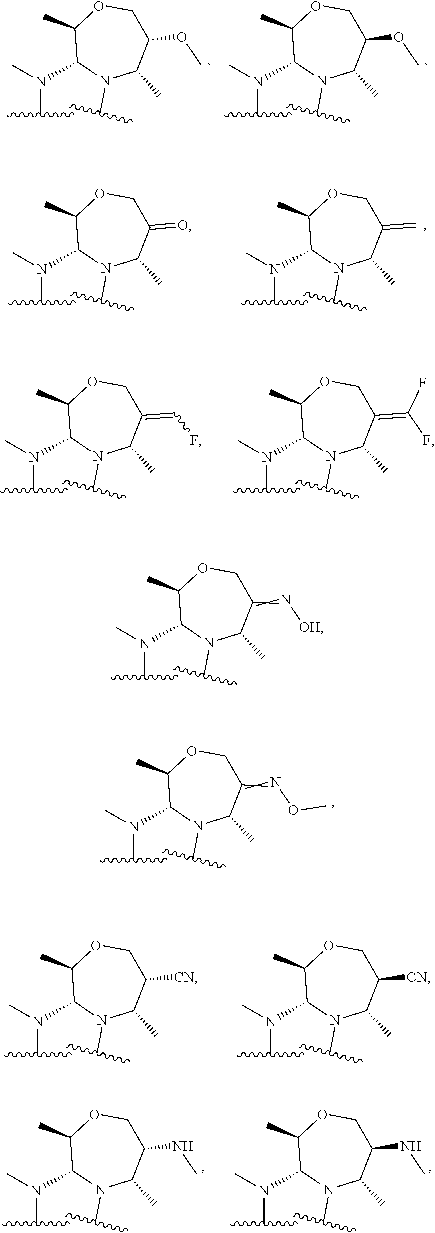

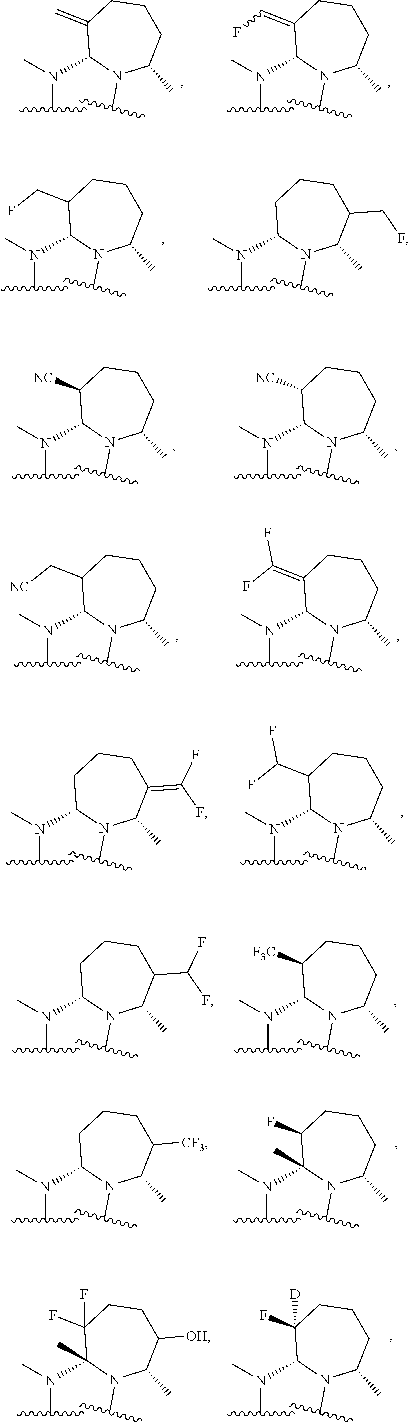

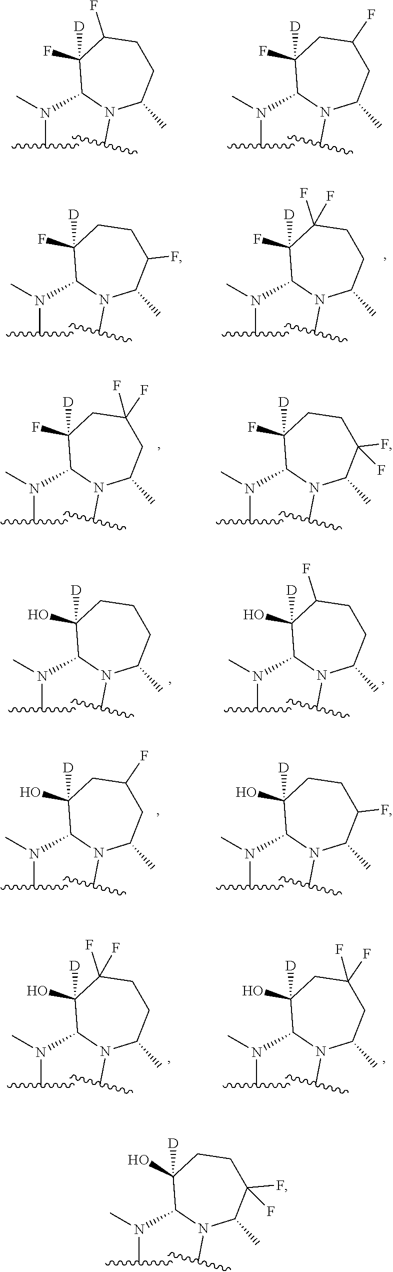

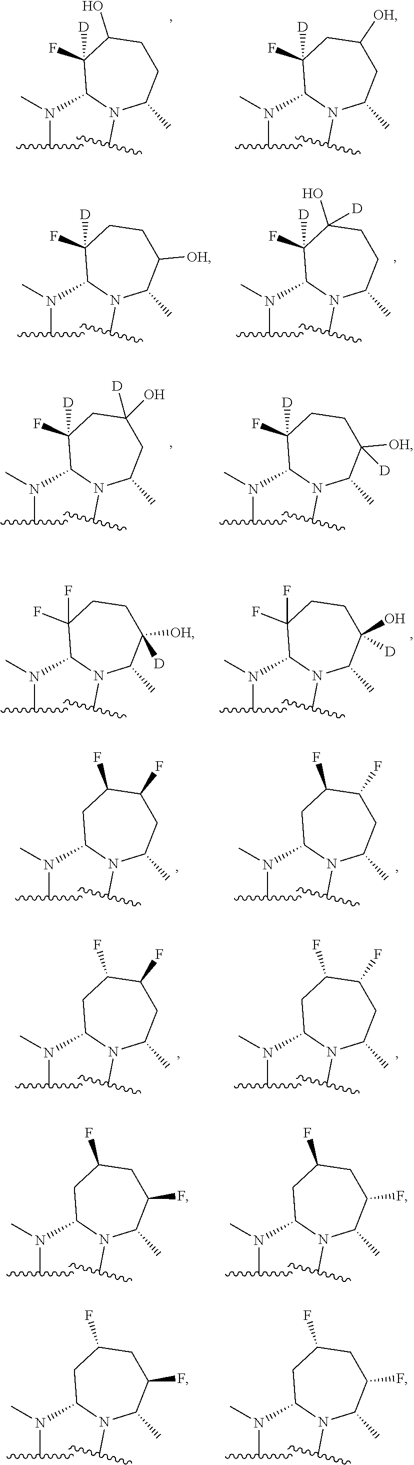

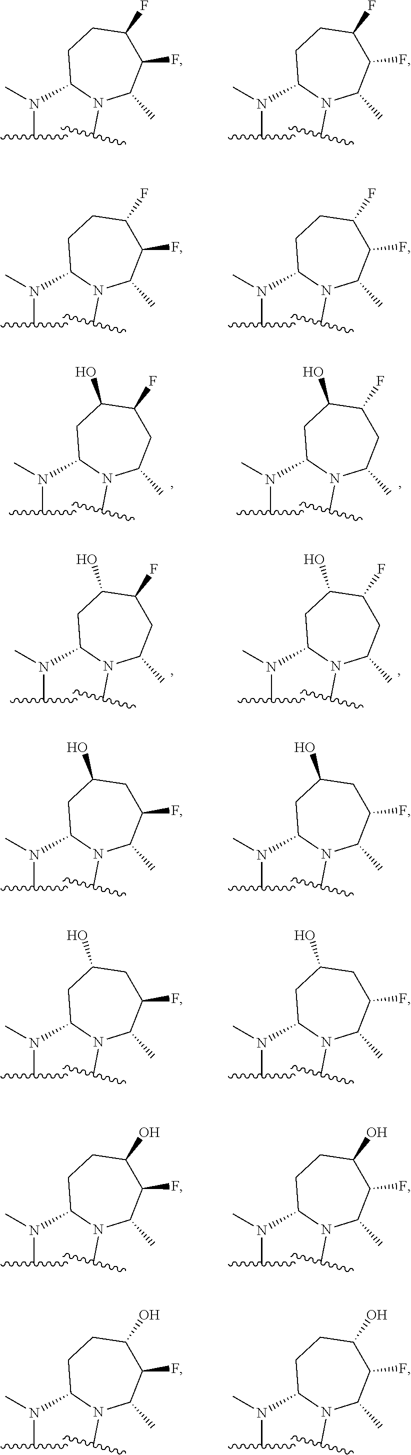

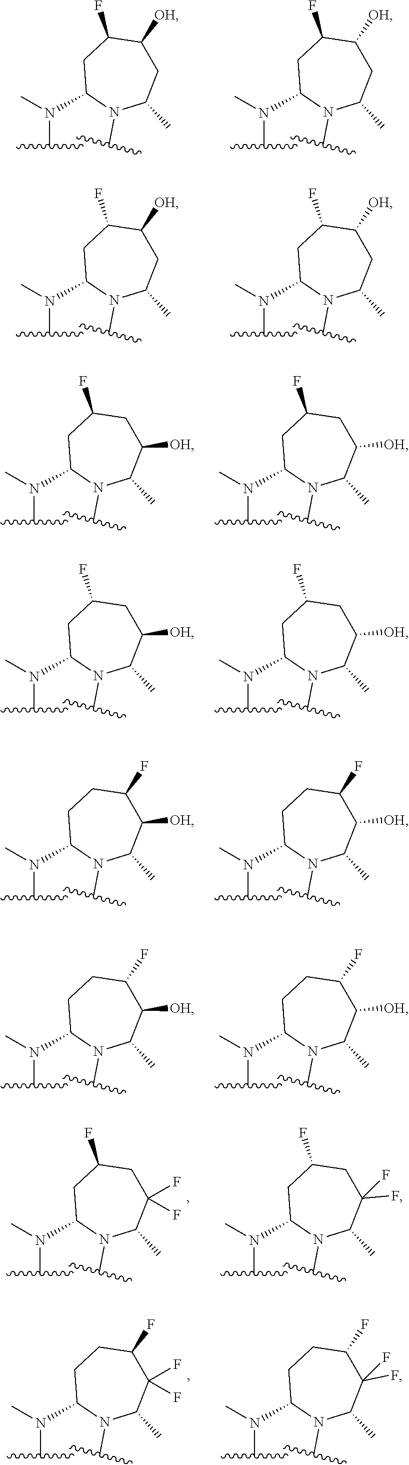

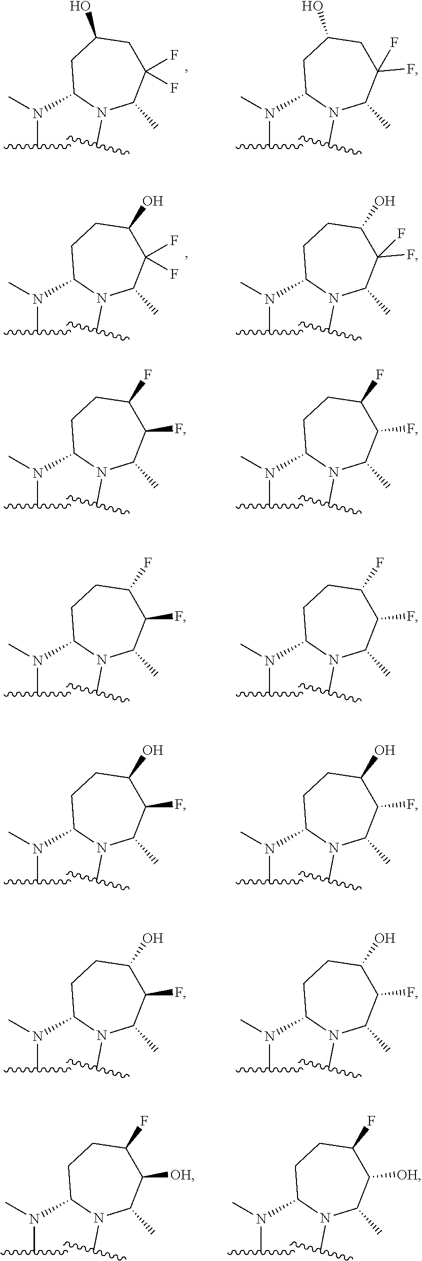

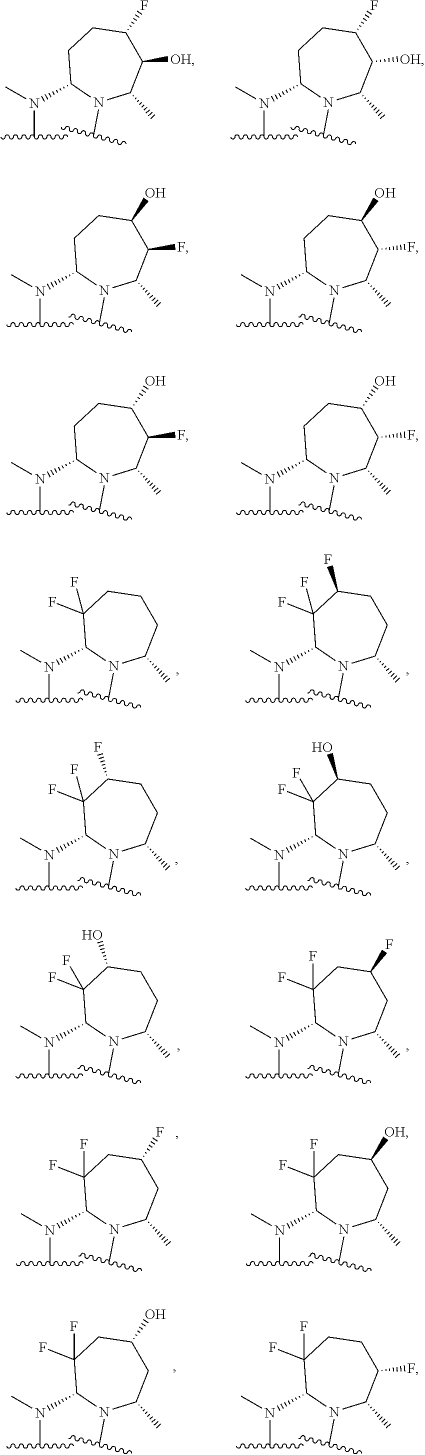

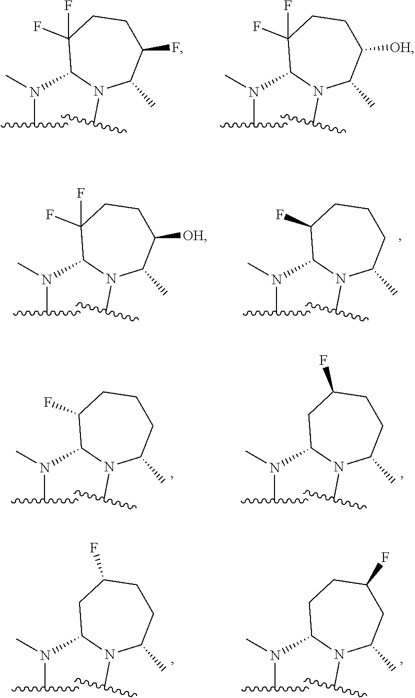

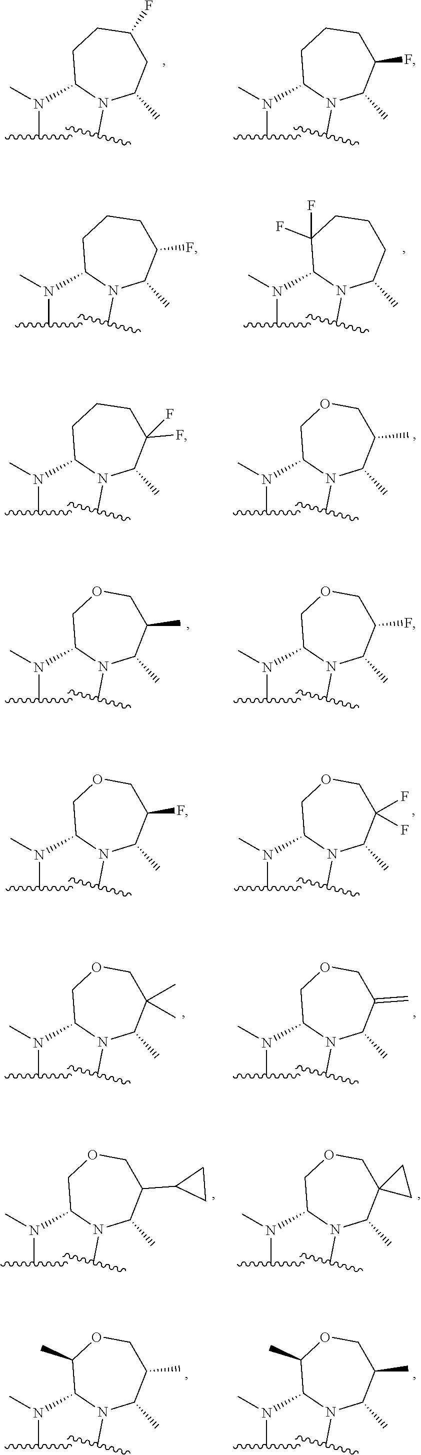

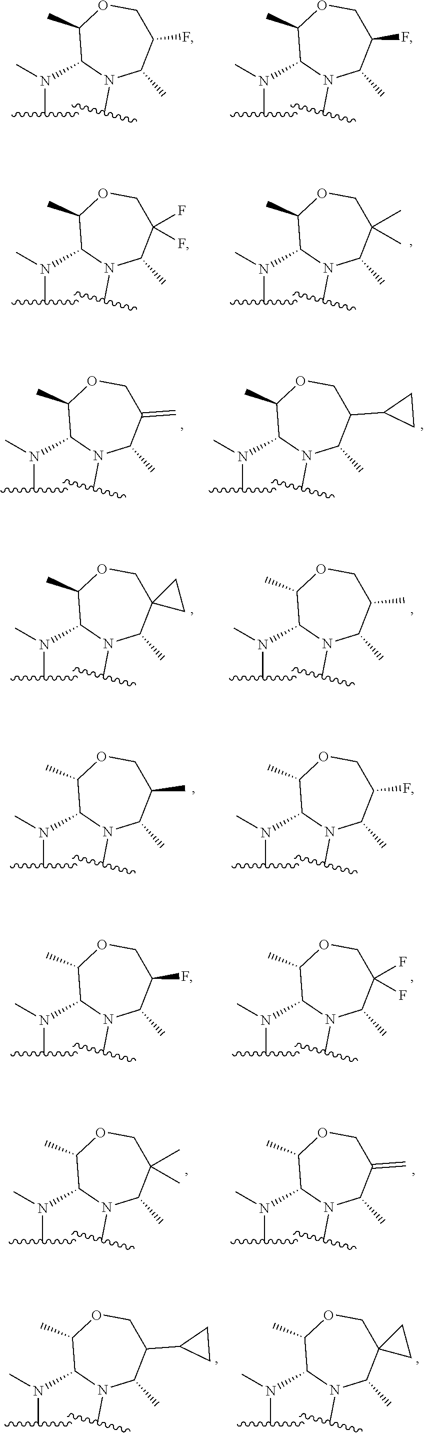

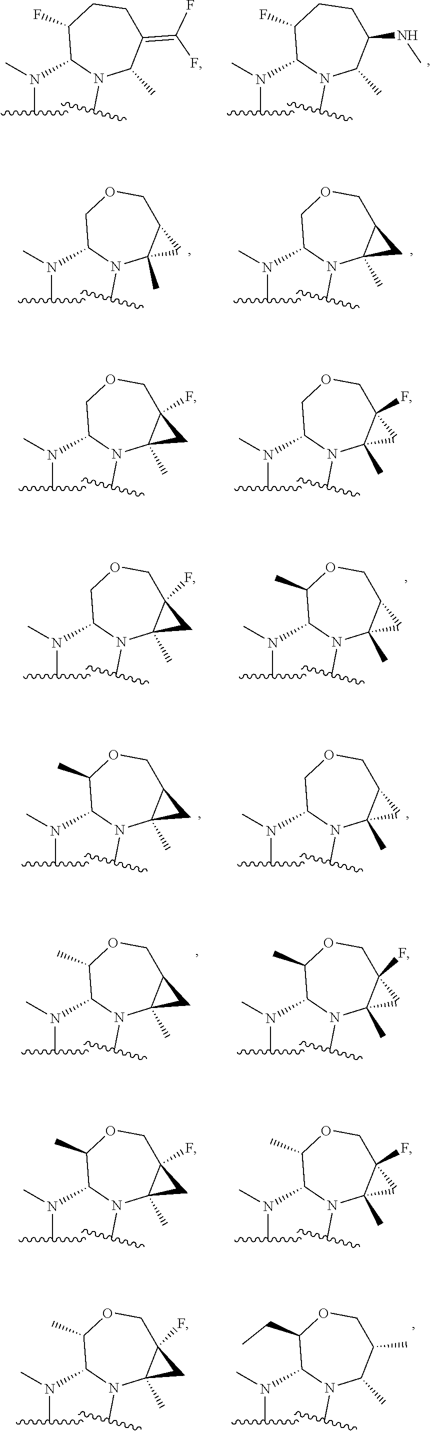

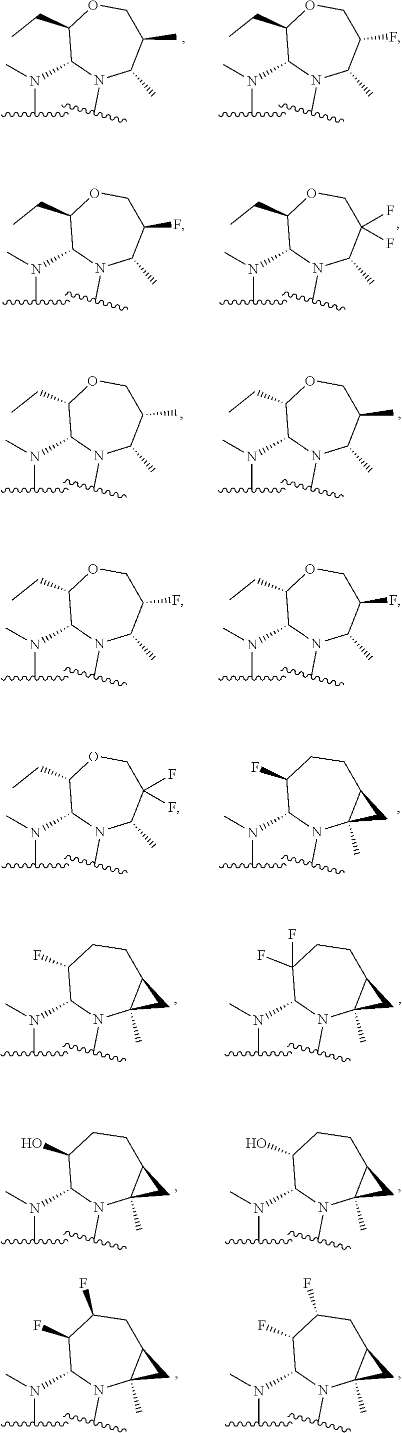

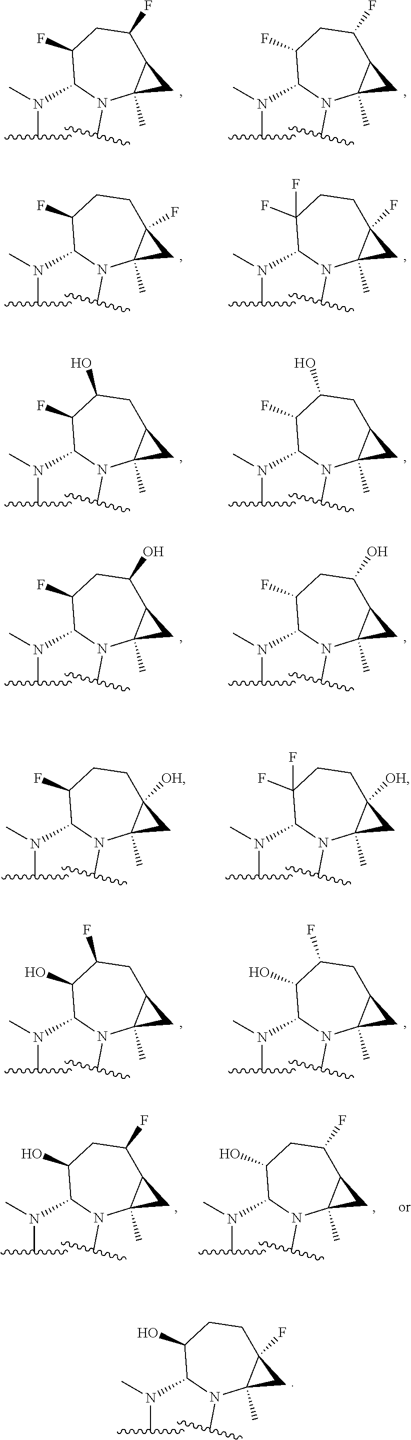

In some embodiments,

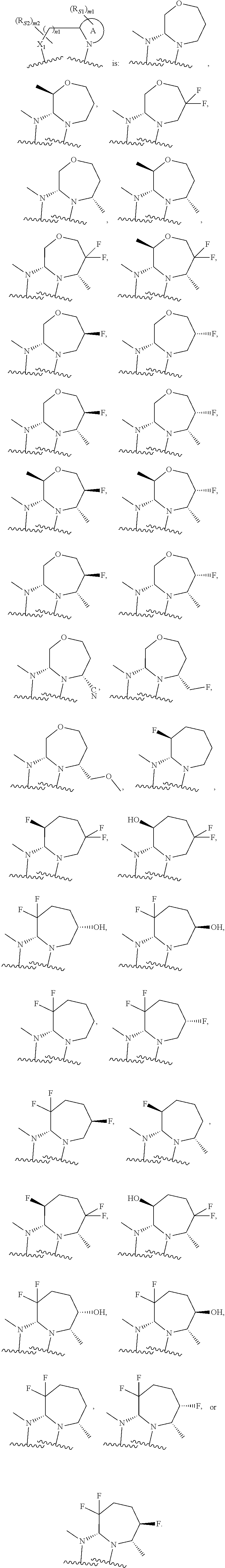

In some embodiments,

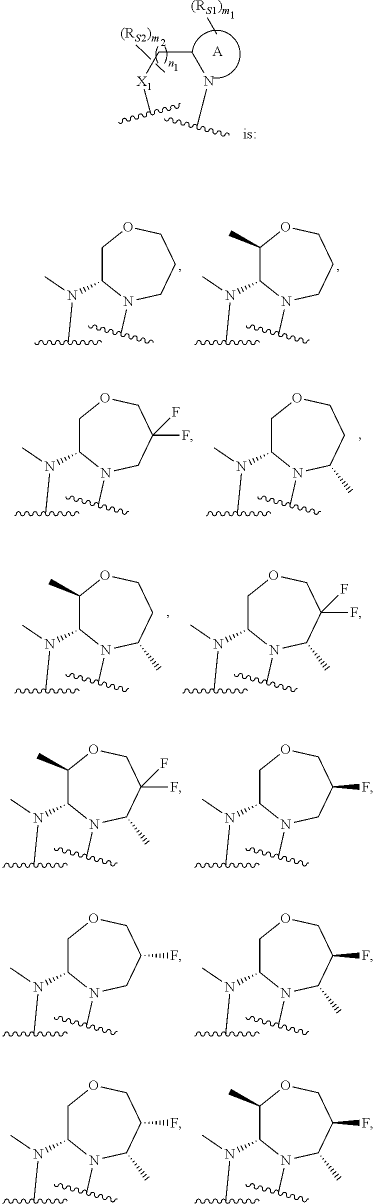

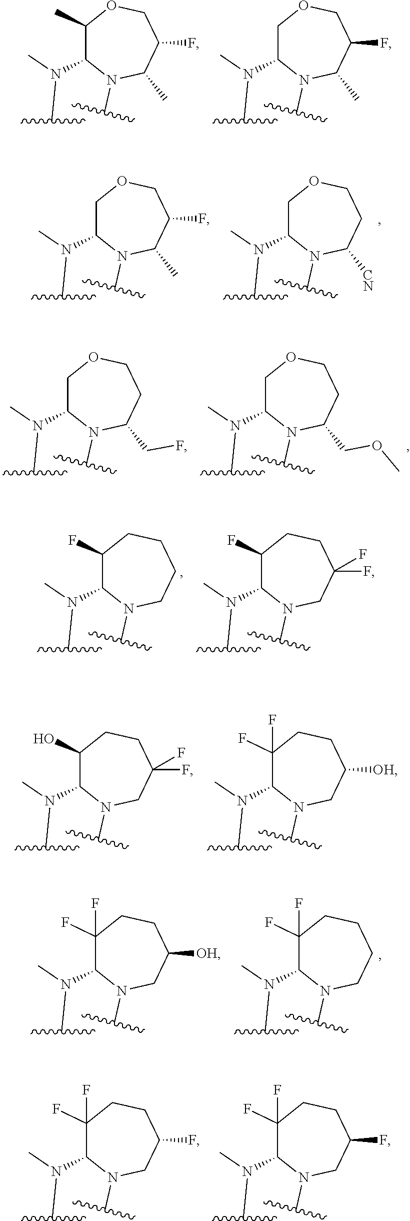

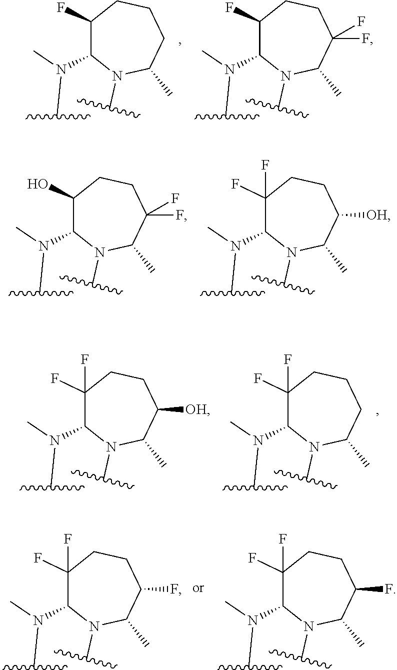

In some embodiments,

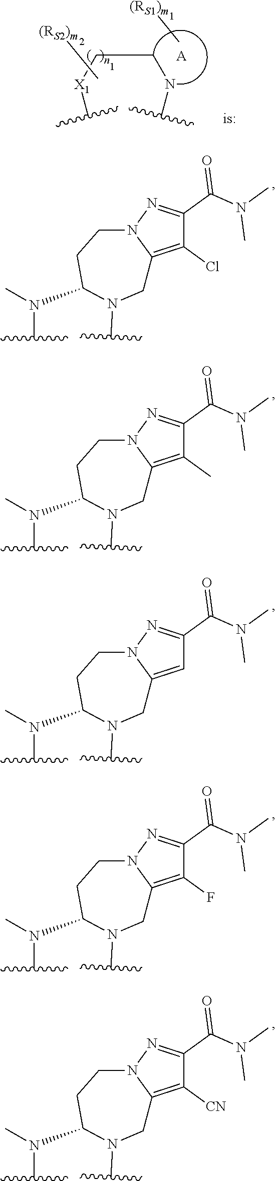

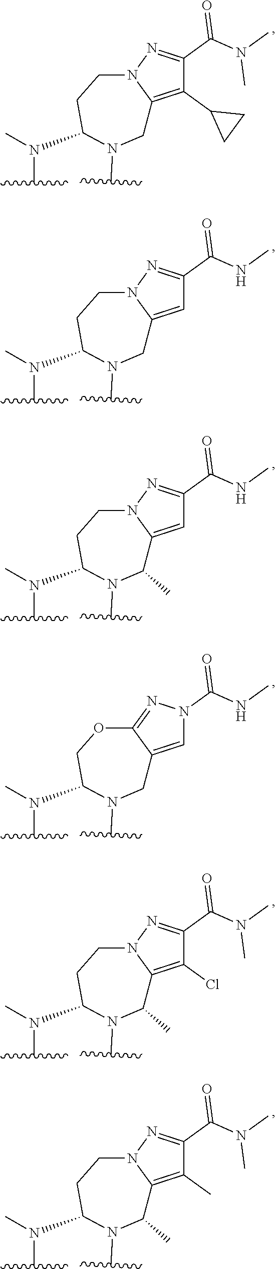

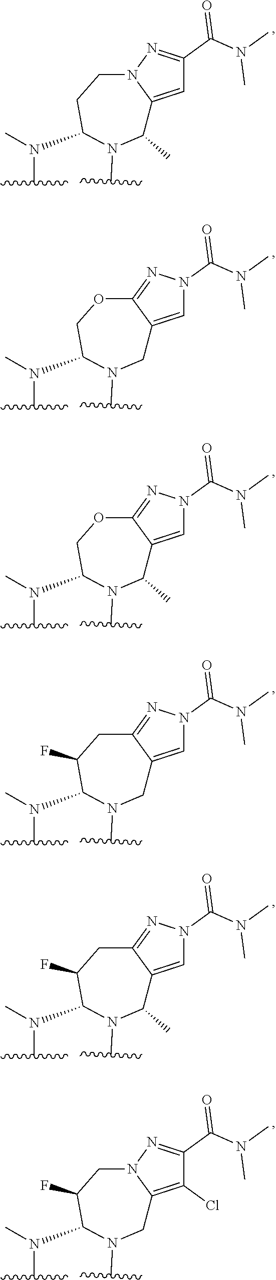

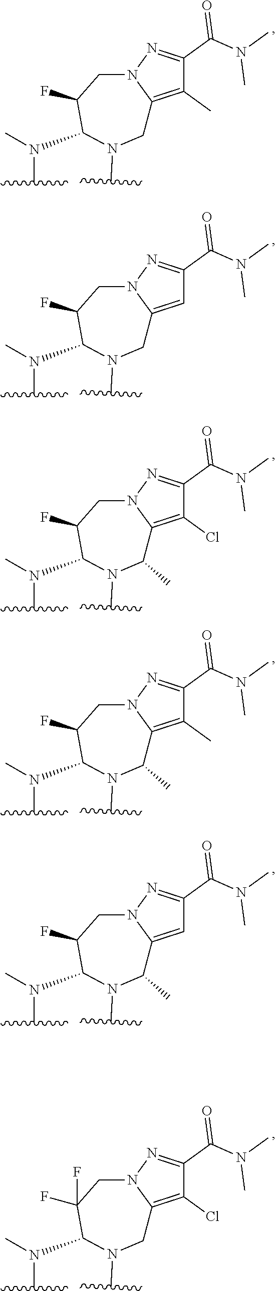

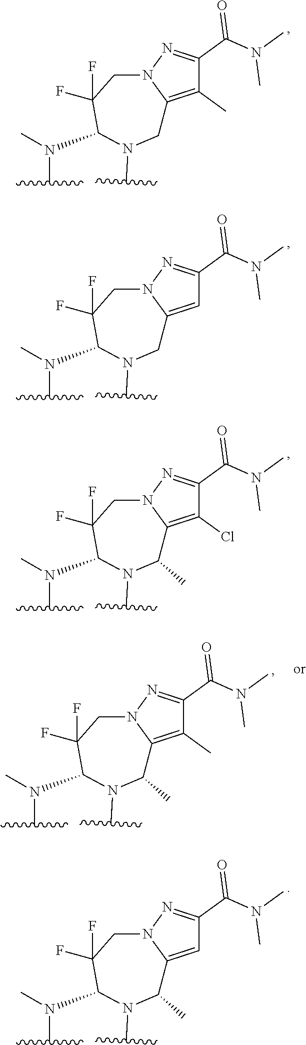

In some embodiments,

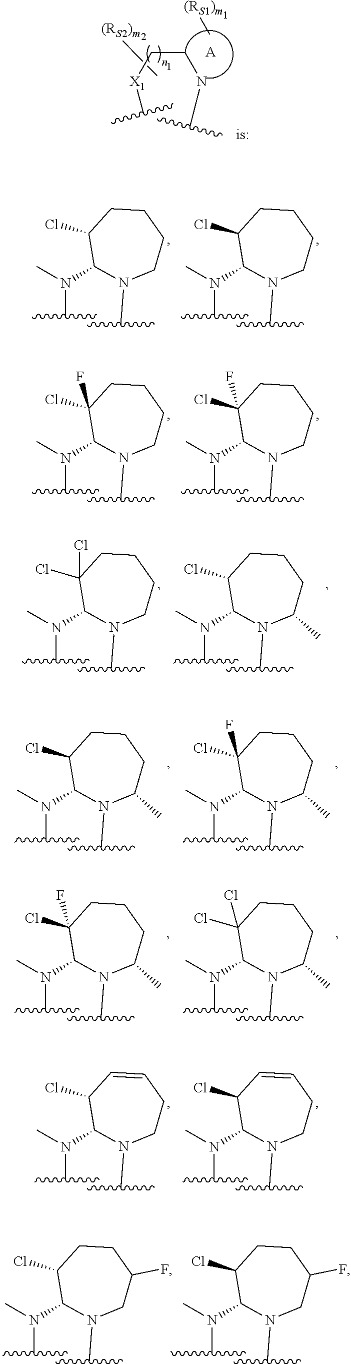

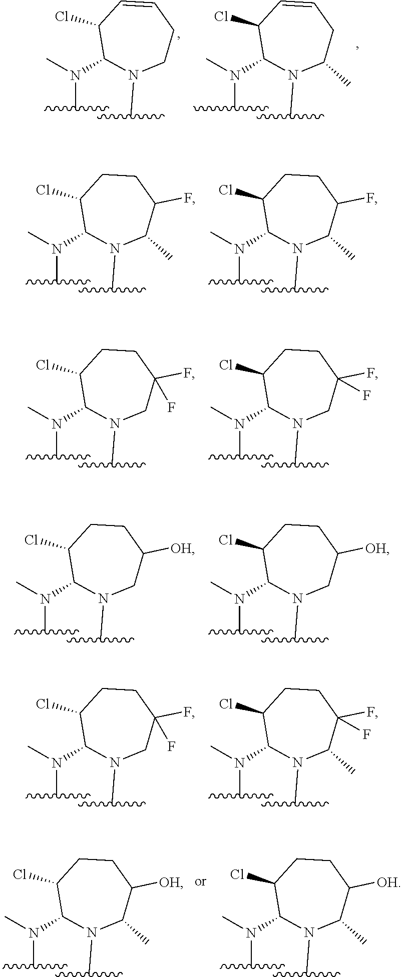

[17]. The compound of any one of [1] to [16], wherein, the compound is any one of the formulas in the Table A:

TABLE A

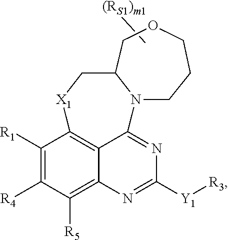

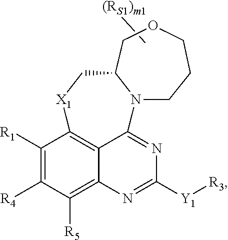

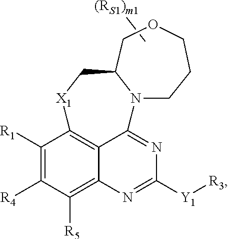

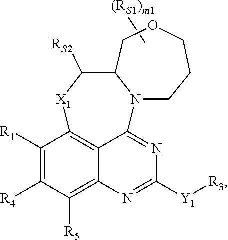

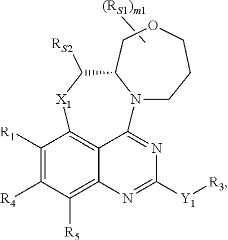

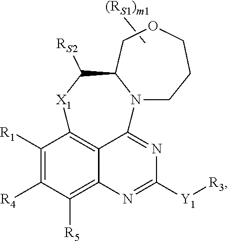

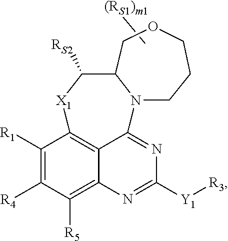

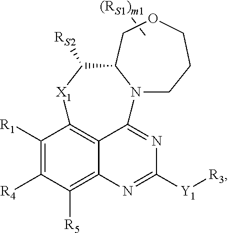

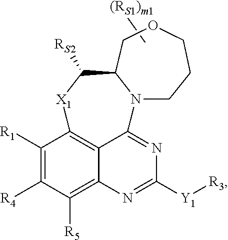

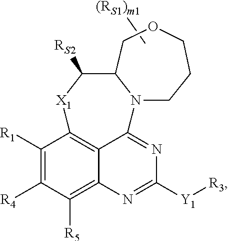

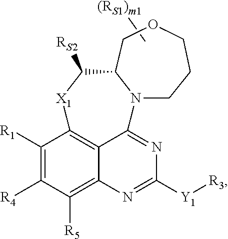

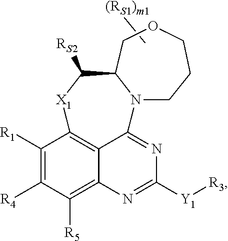

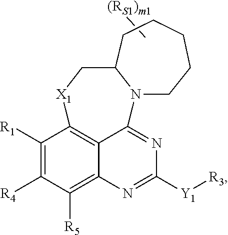

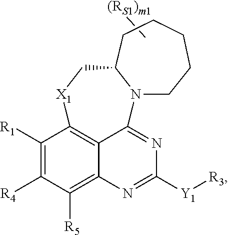

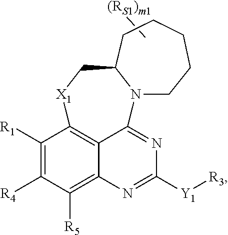

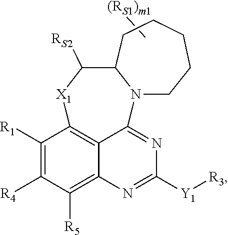

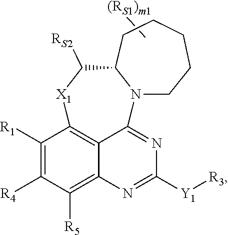

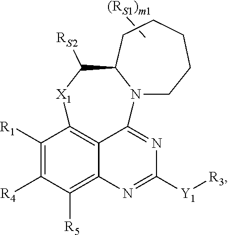

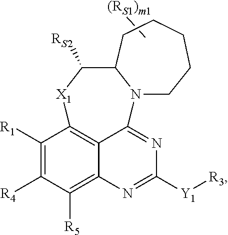

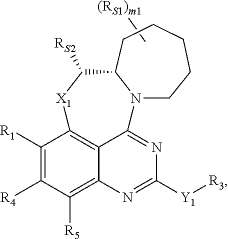

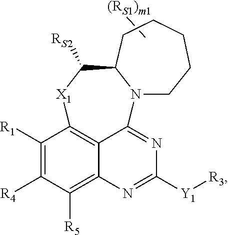

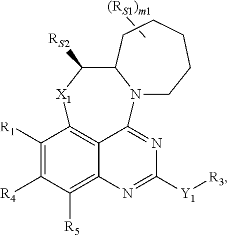

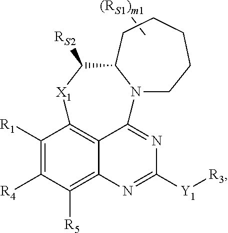

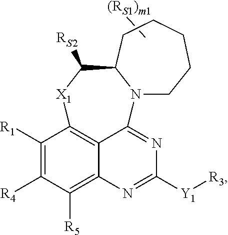

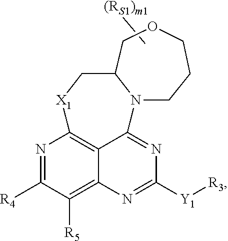

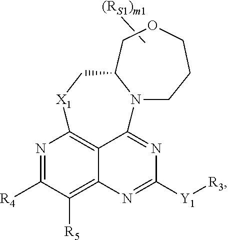

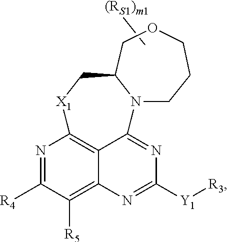

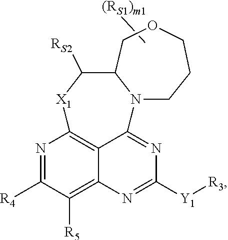

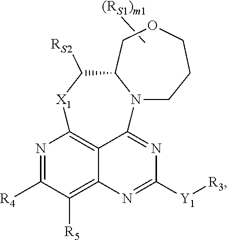

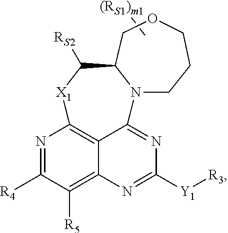

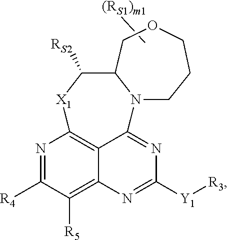

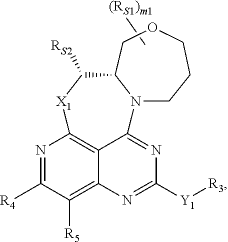

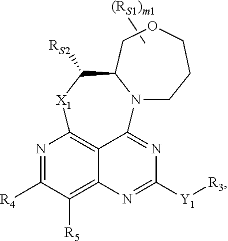

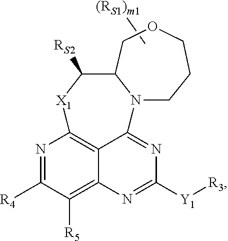

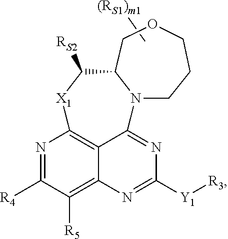

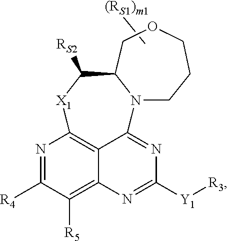

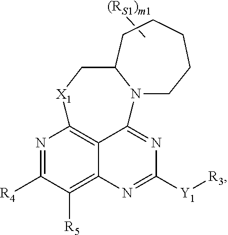

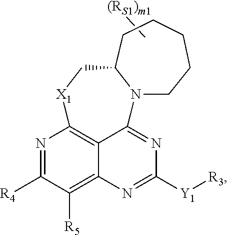

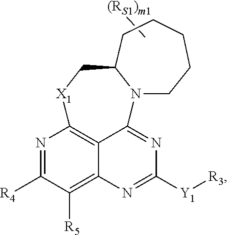

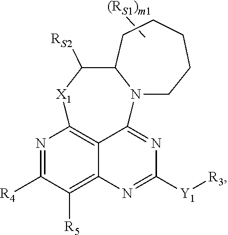

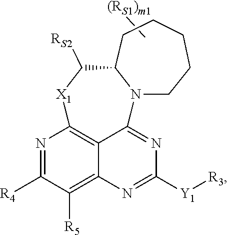

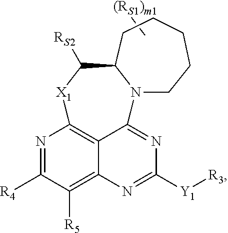

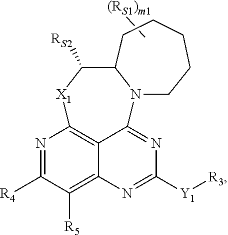

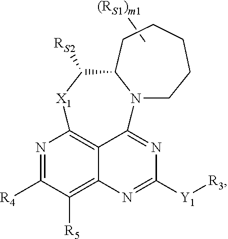

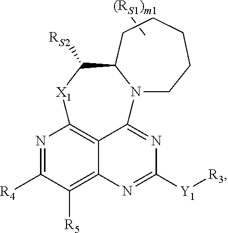

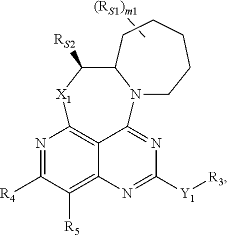

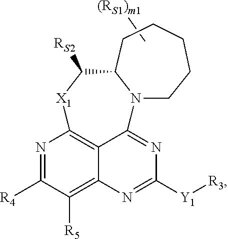

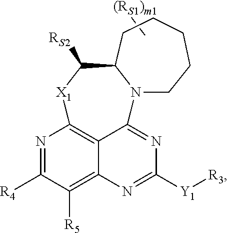

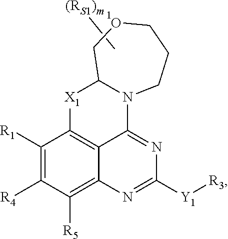

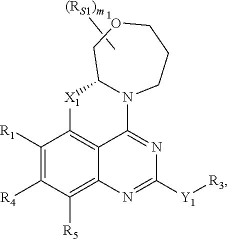

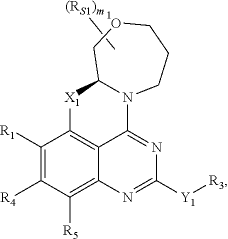

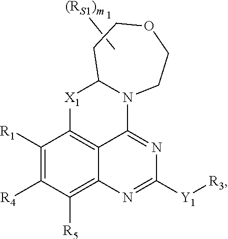

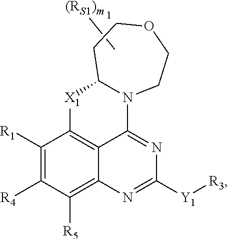

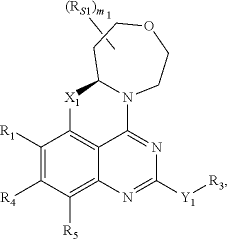

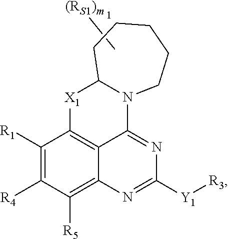

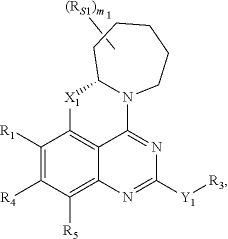

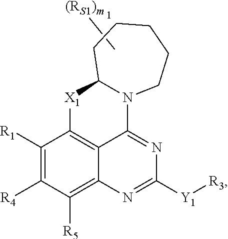

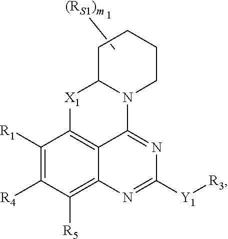

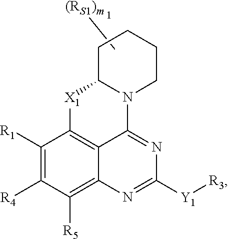

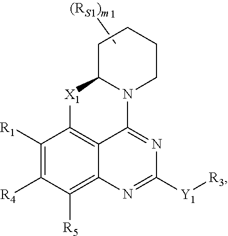

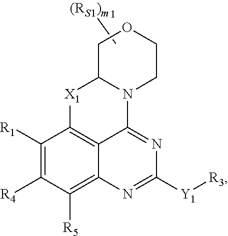

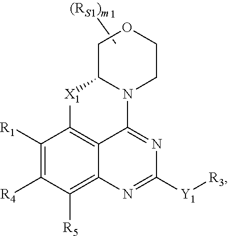

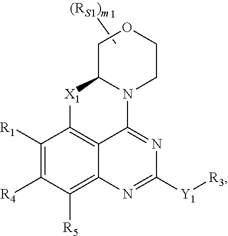

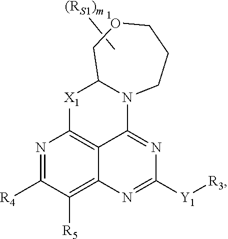

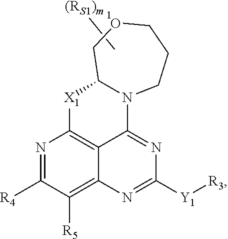

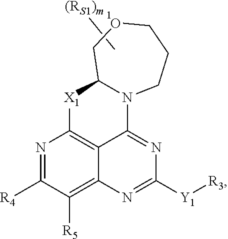

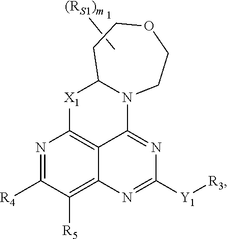

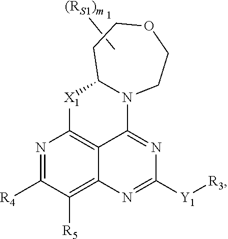

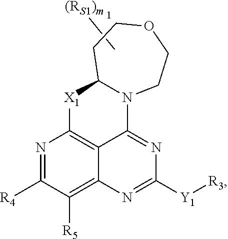

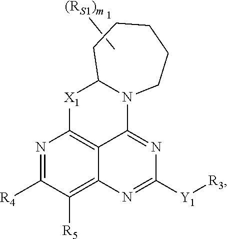

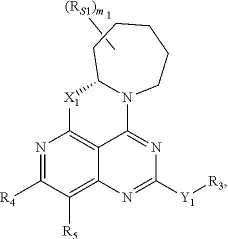

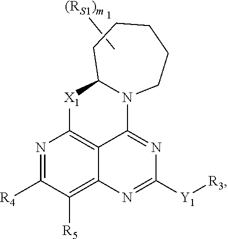

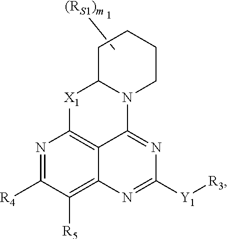

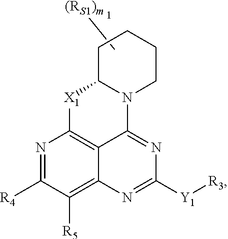

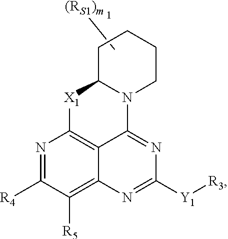

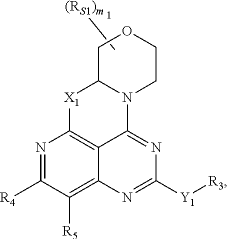

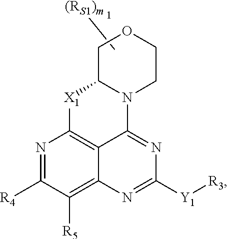

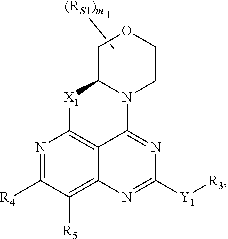

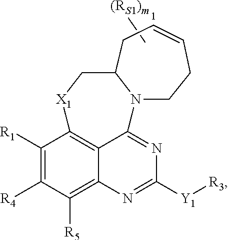

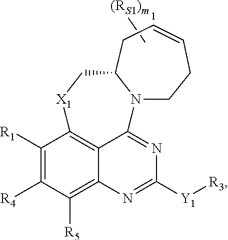

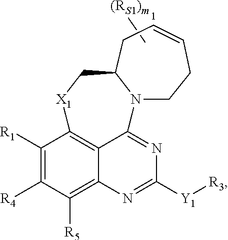

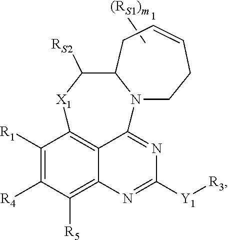

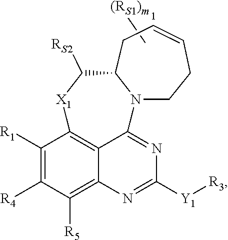

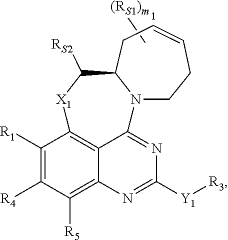

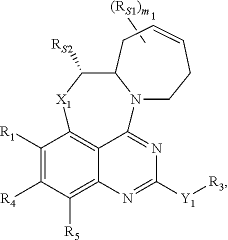

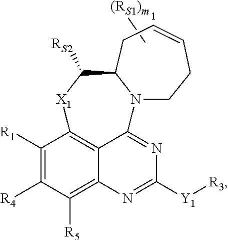

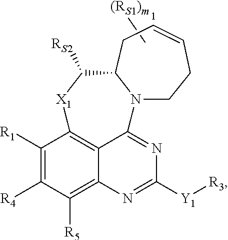

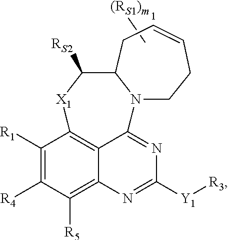

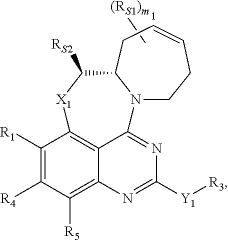

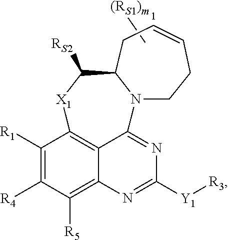

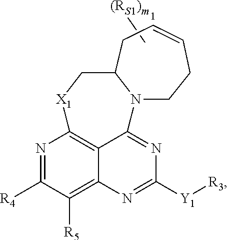

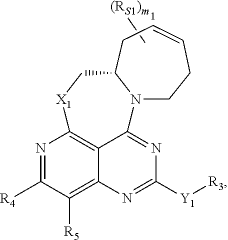

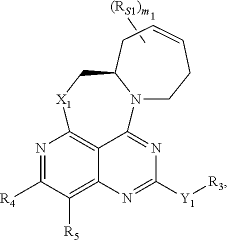

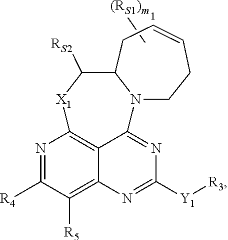

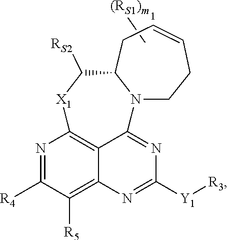

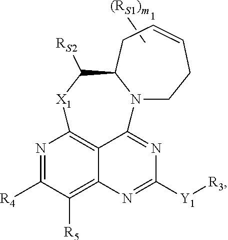

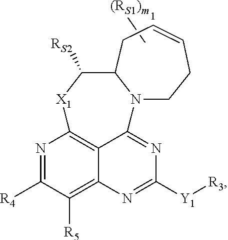

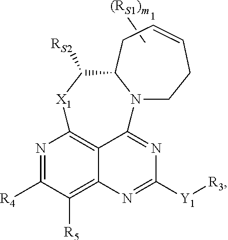

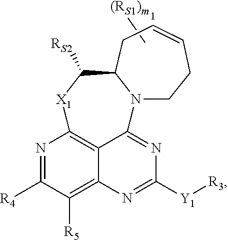

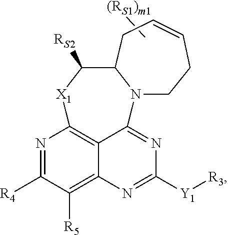

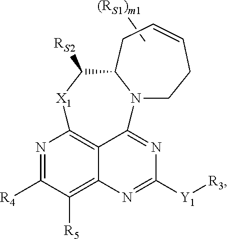

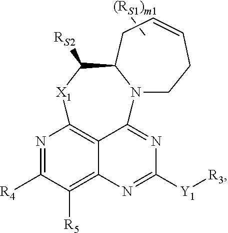

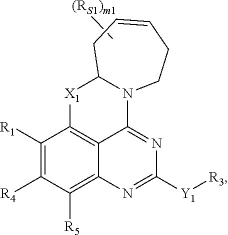

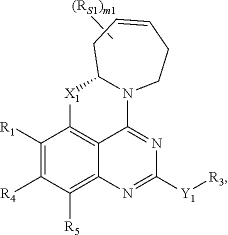

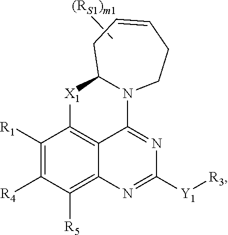

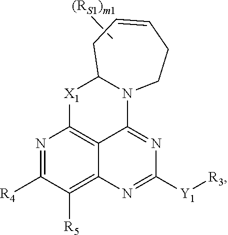

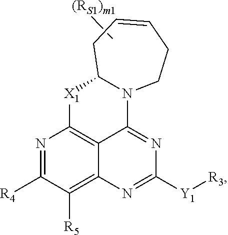

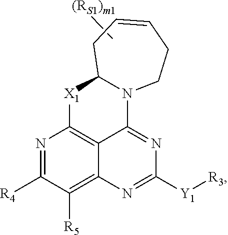

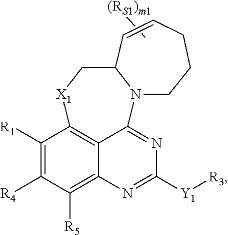

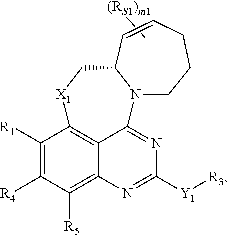

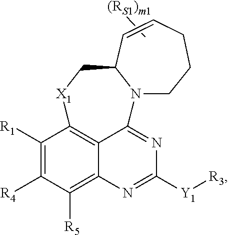

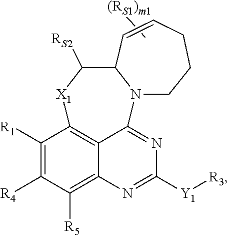

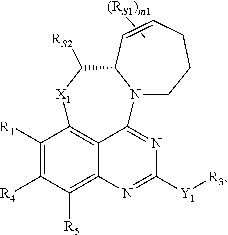

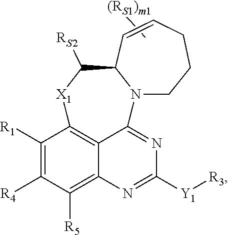

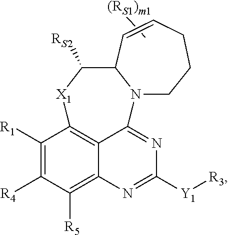

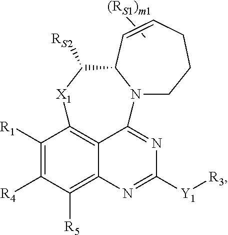

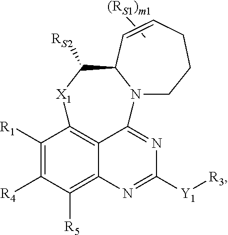

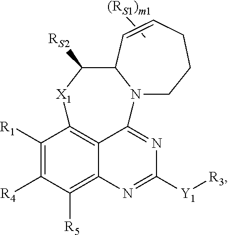

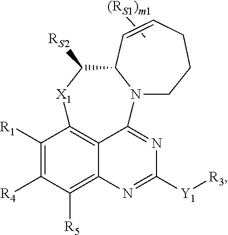

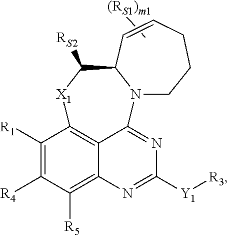

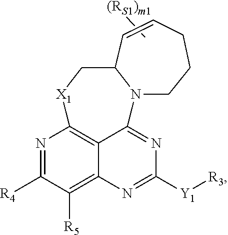

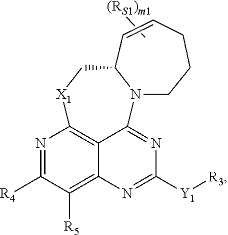

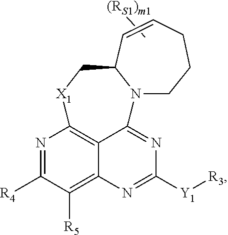

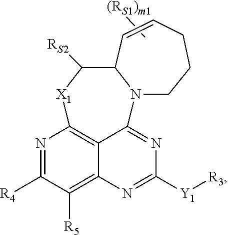

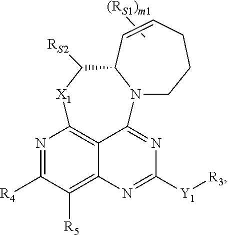

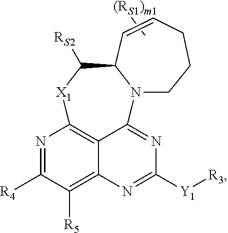

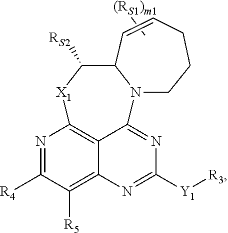

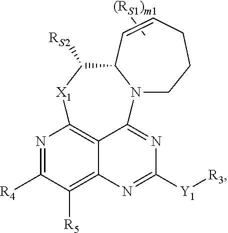

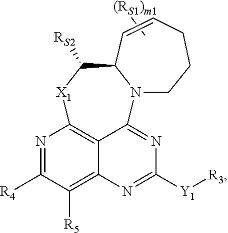

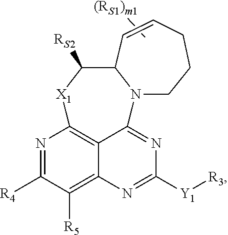

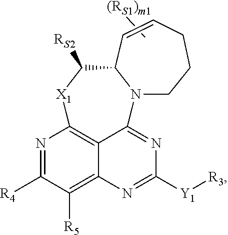

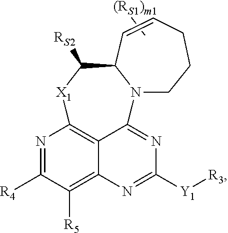

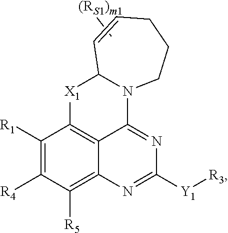

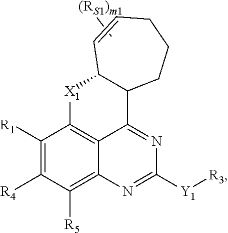

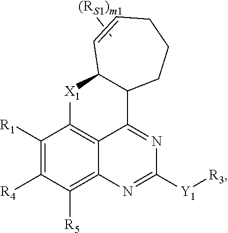

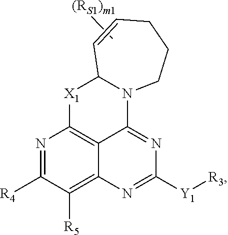

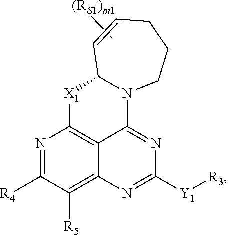

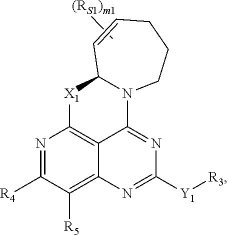

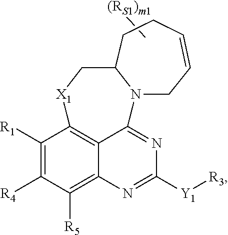

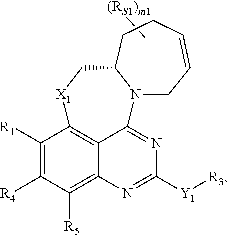

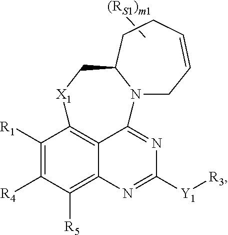

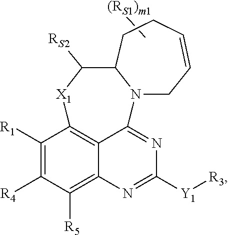

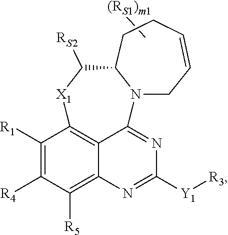

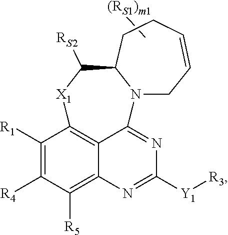

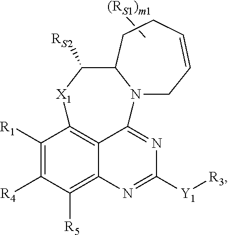

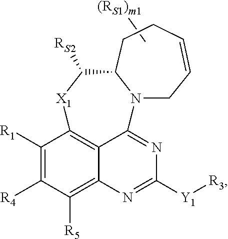

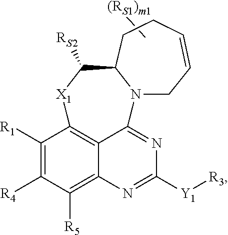

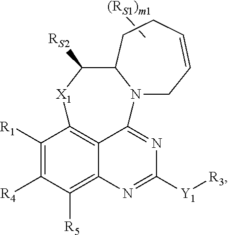

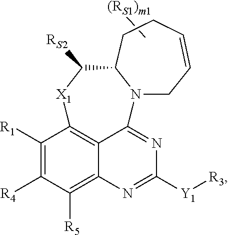

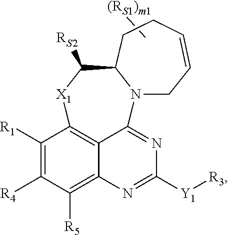

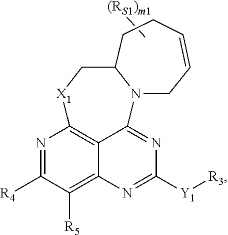

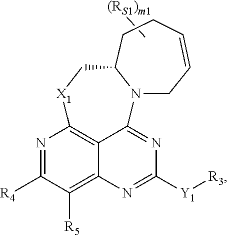

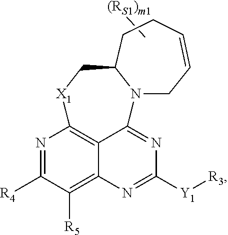

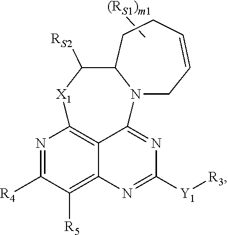

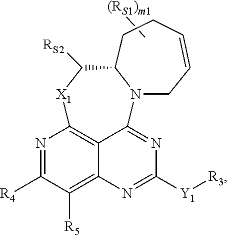

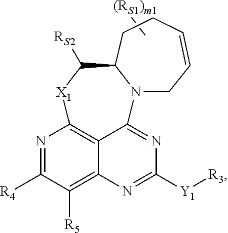

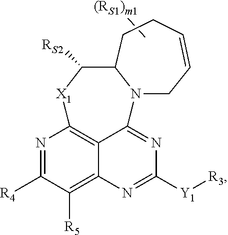

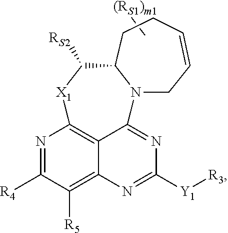

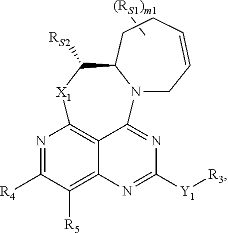

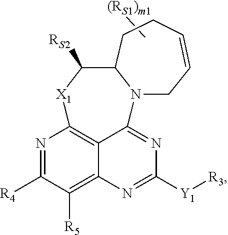

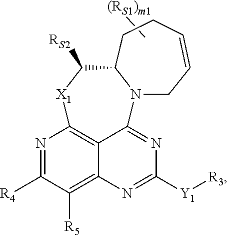

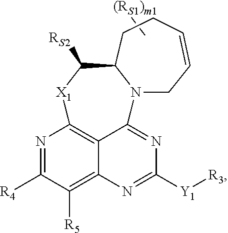

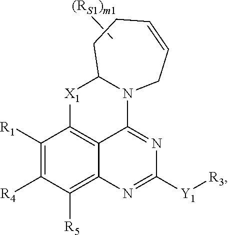

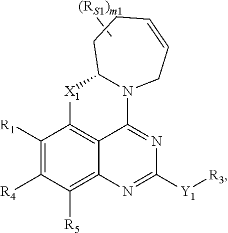

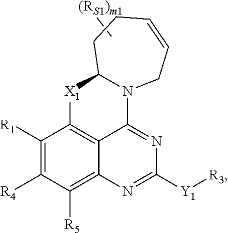

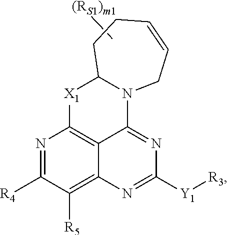

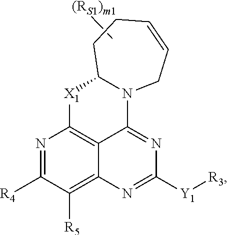

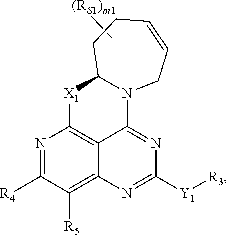

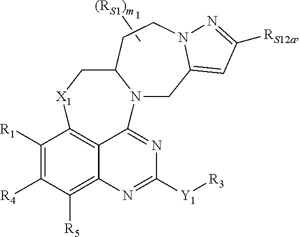

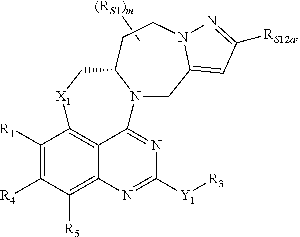

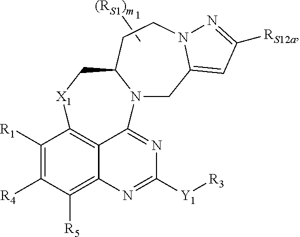

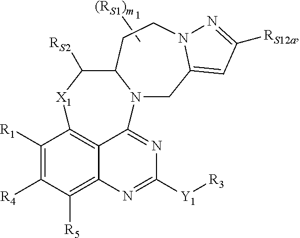

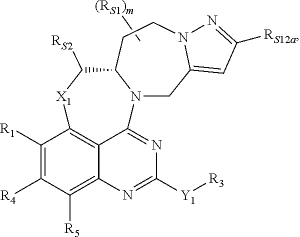

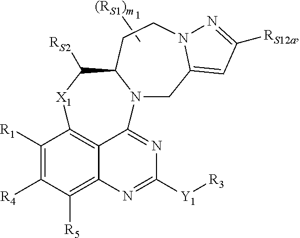

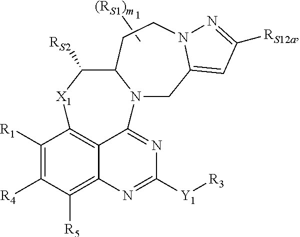

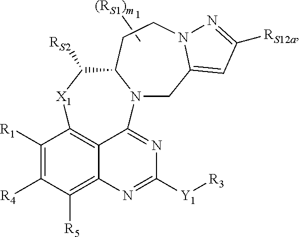

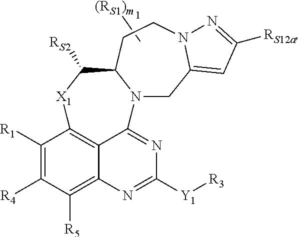

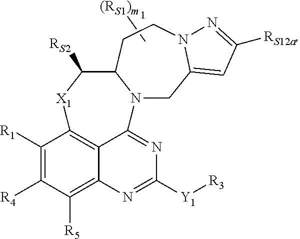

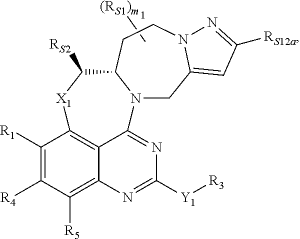

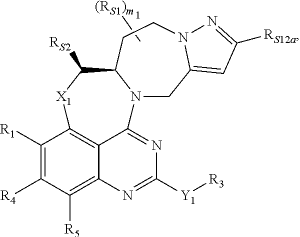

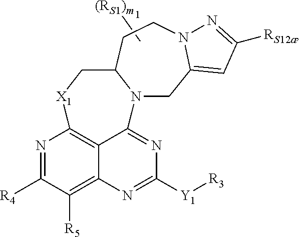

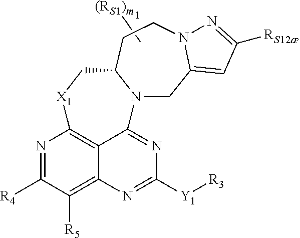

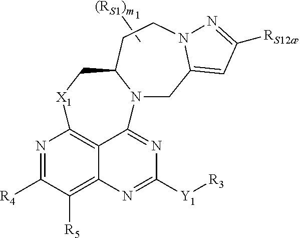

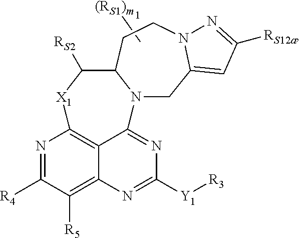

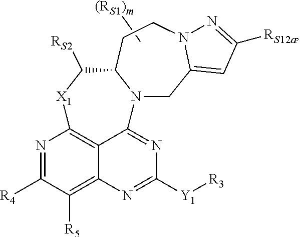

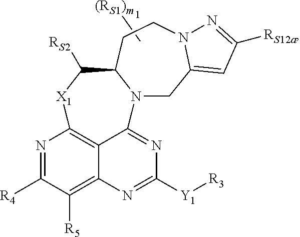

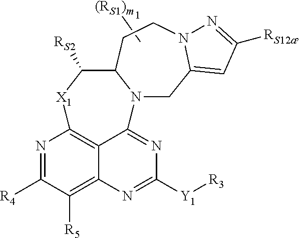

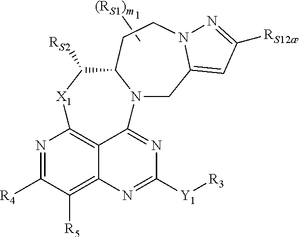

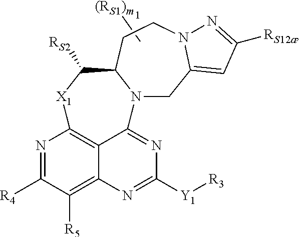

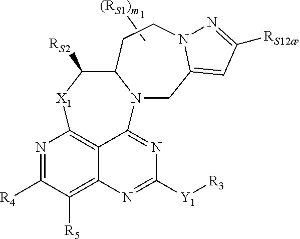

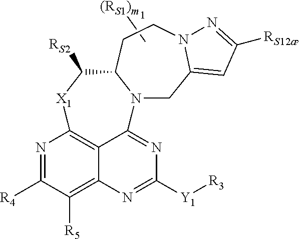

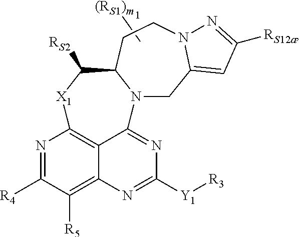

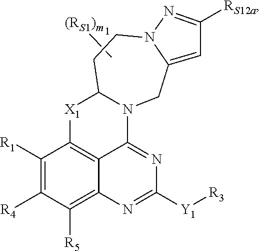

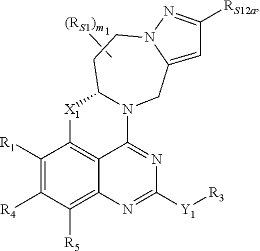

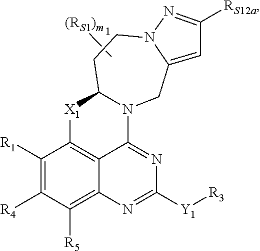

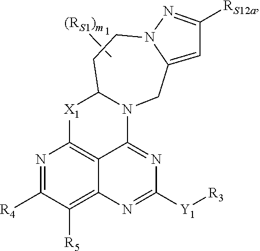

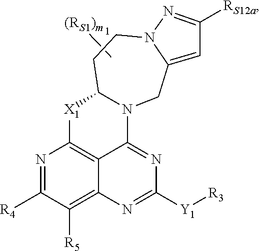

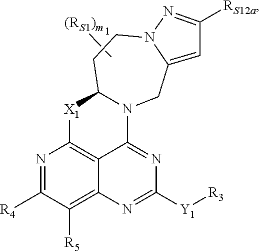

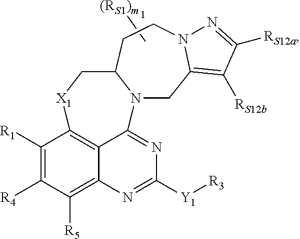

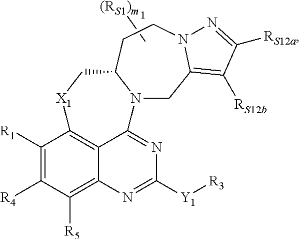

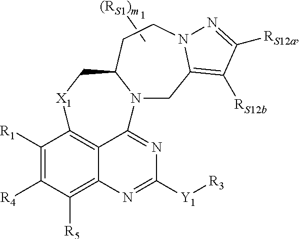

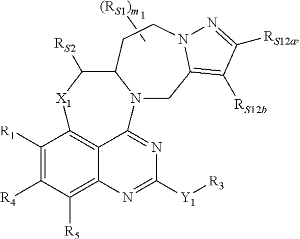

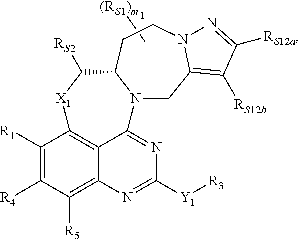

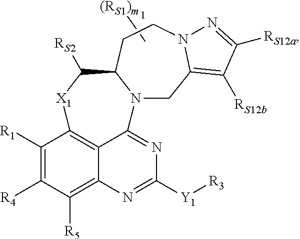

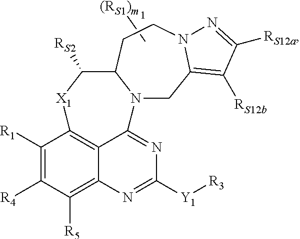

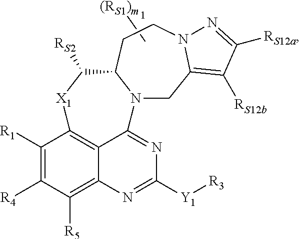

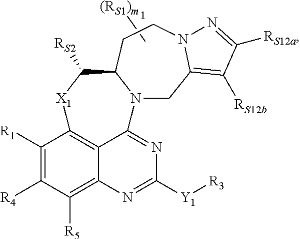

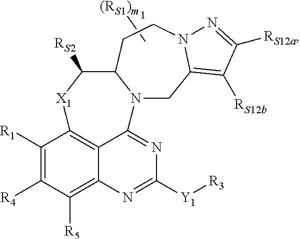

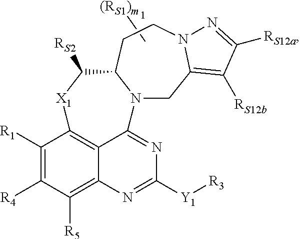

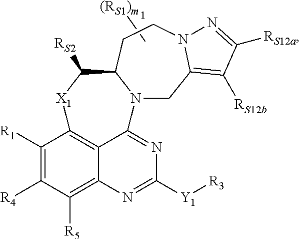

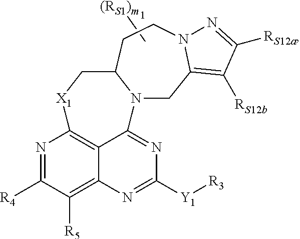

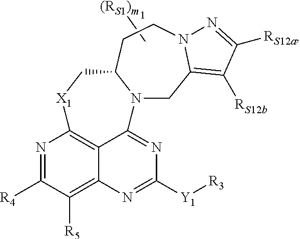

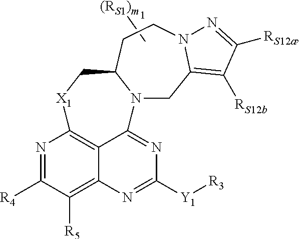

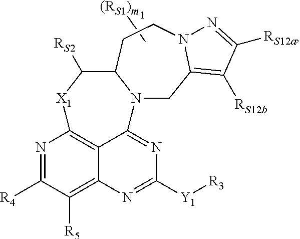

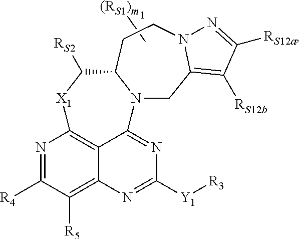

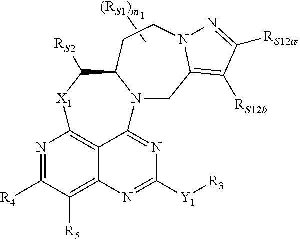

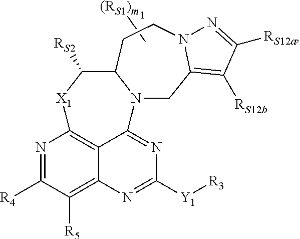

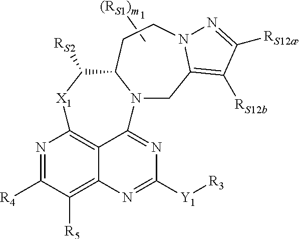

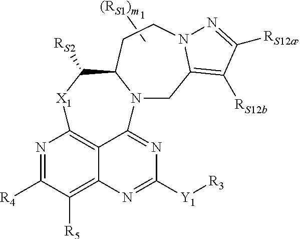

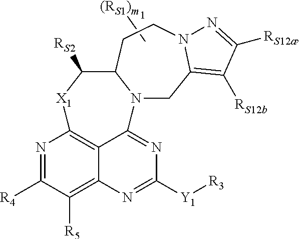

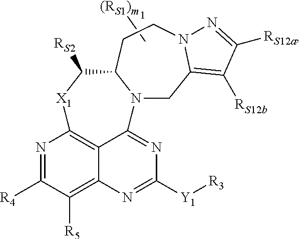

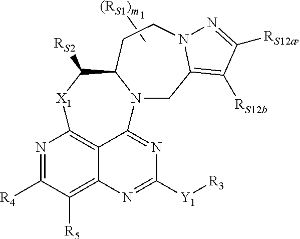

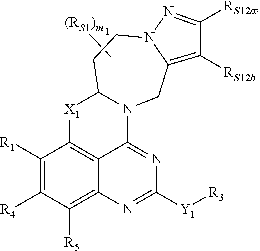

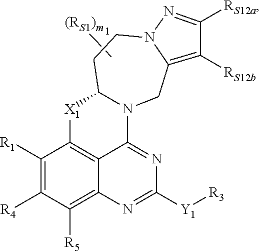

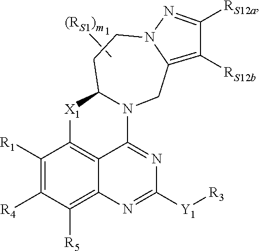

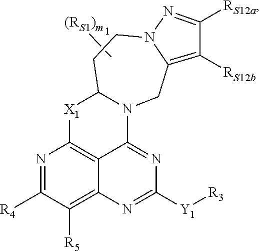

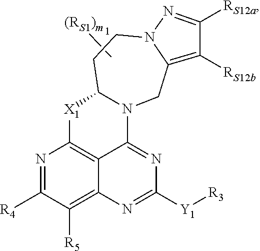

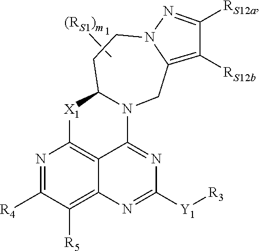

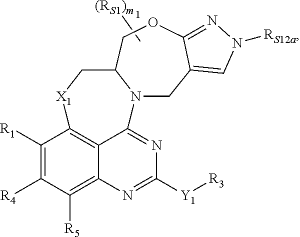

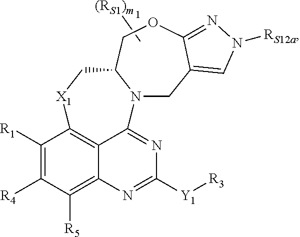

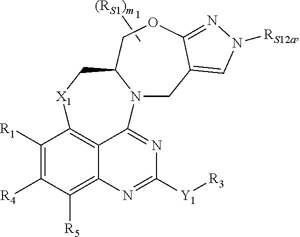

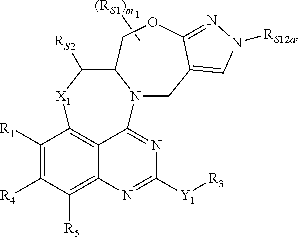

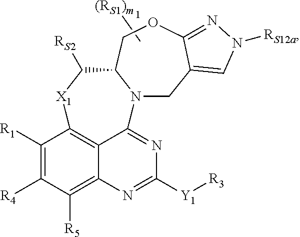

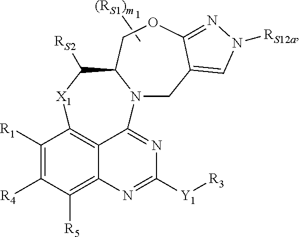

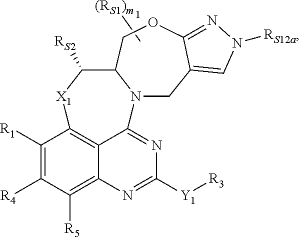

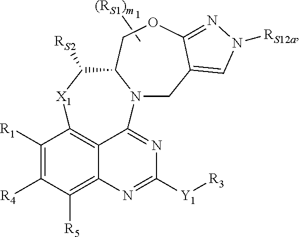

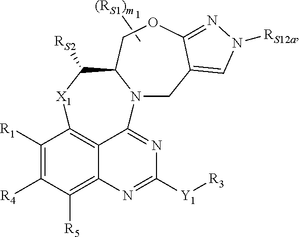

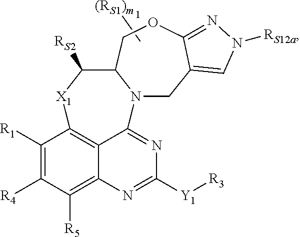

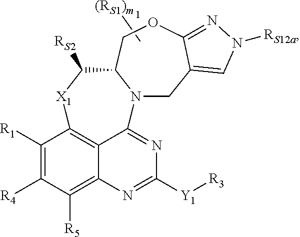

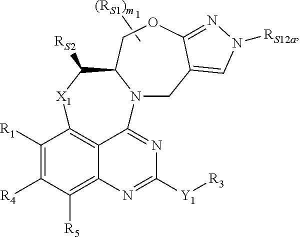

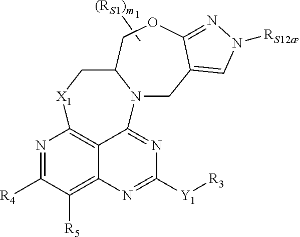

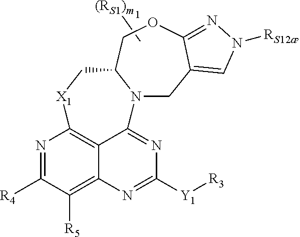

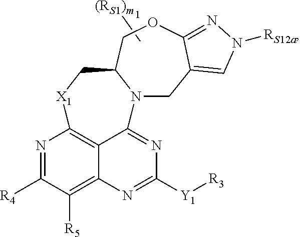

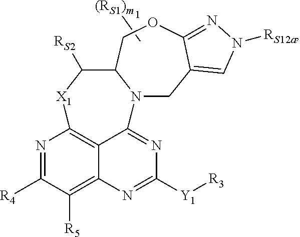

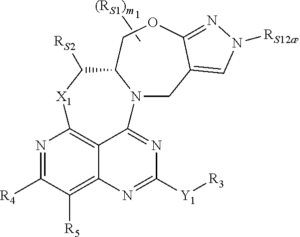

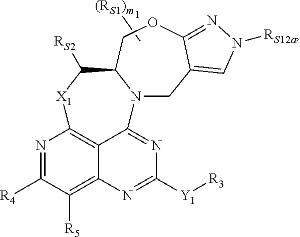

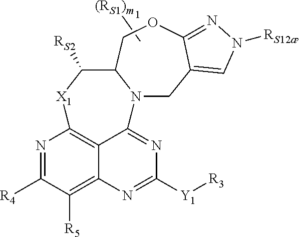

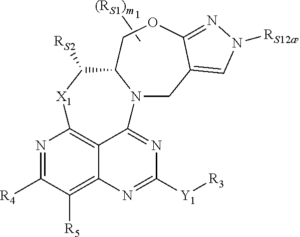

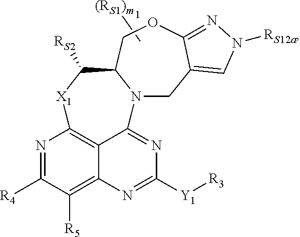

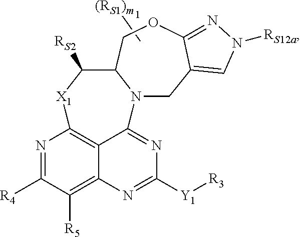

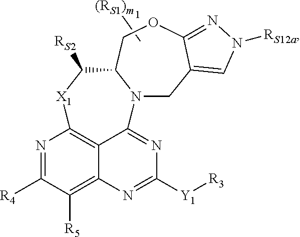

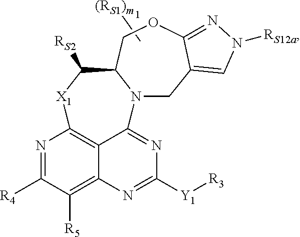

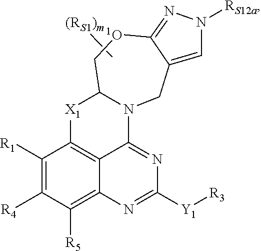

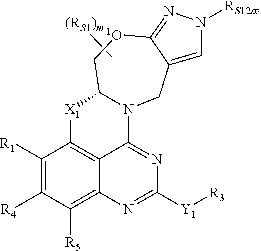

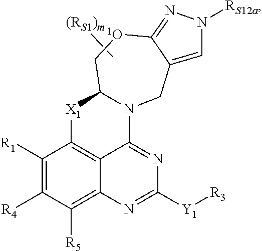

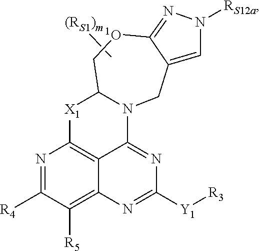

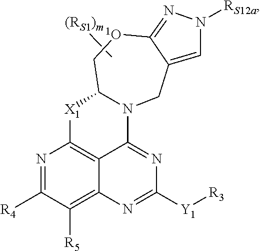

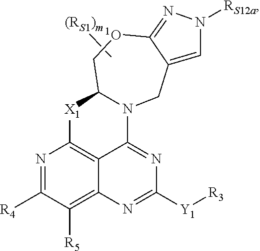

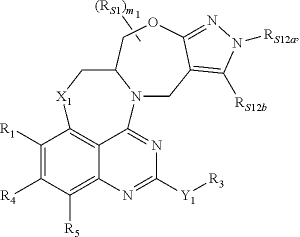

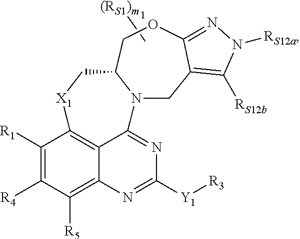

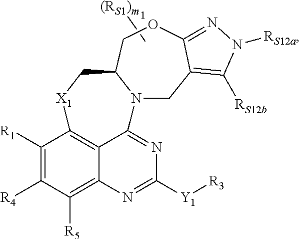

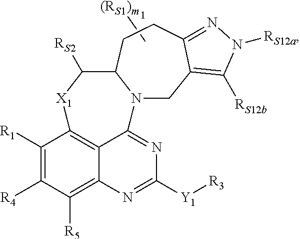

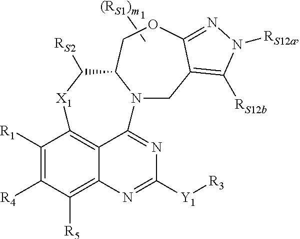

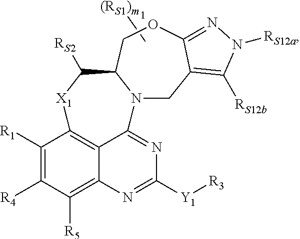

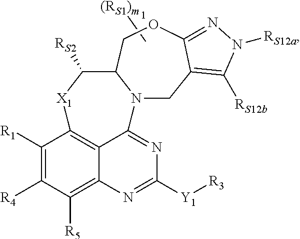

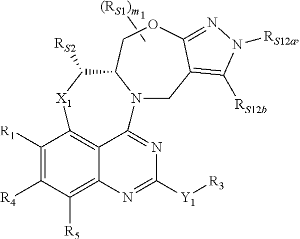

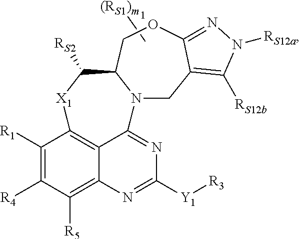

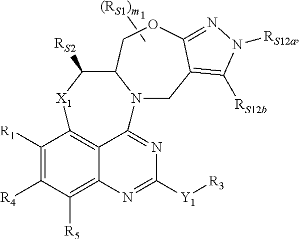

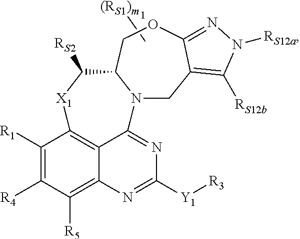

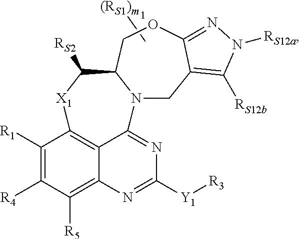

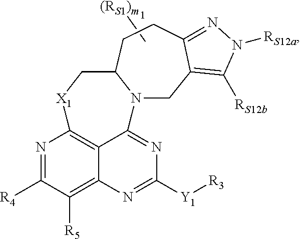

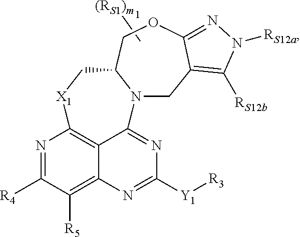

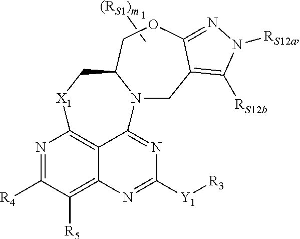

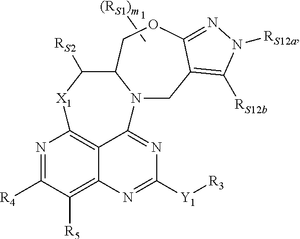

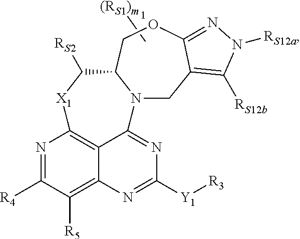

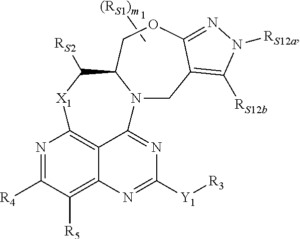

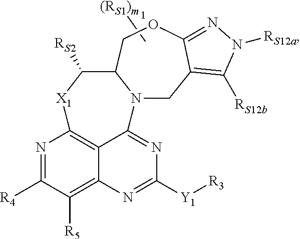

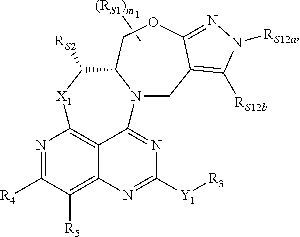

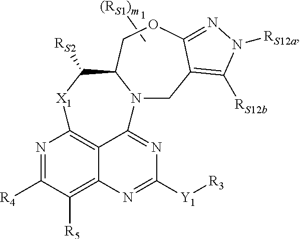

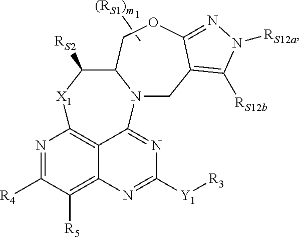

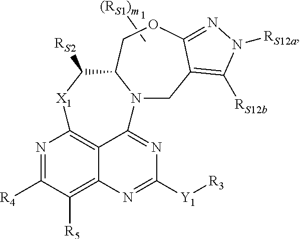

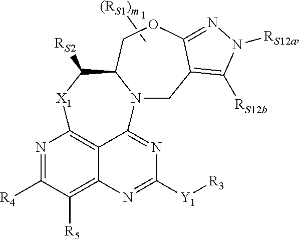

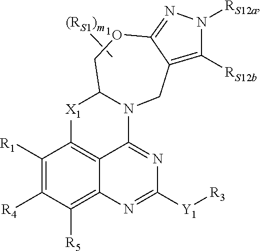

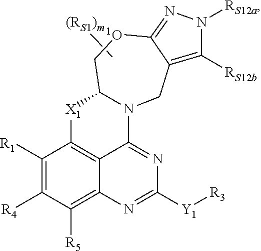

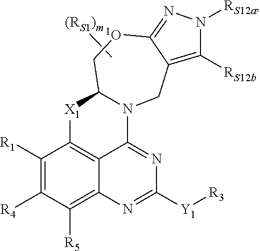

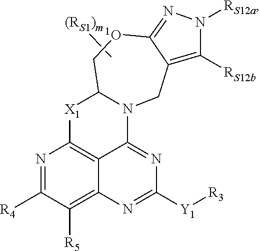

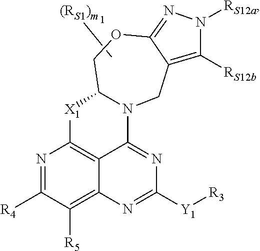

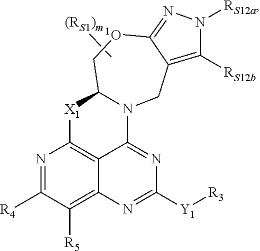

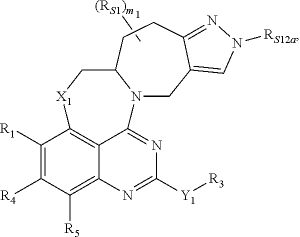

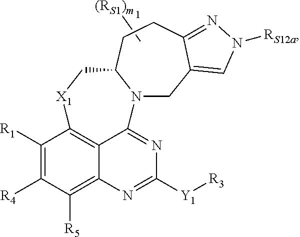

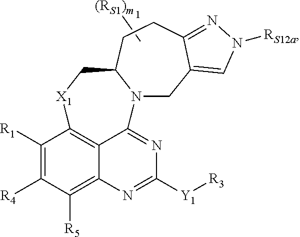

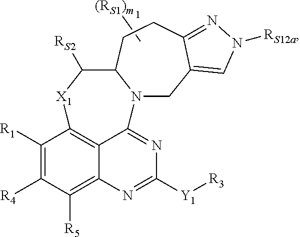

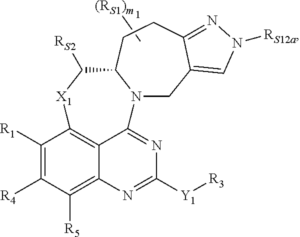

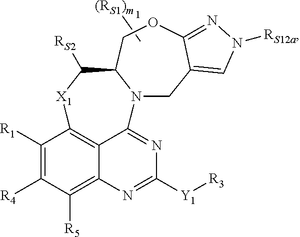

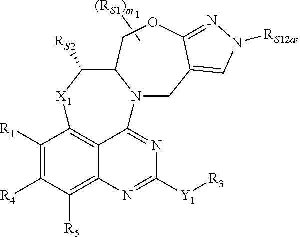

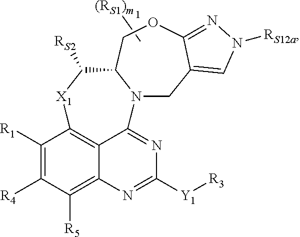

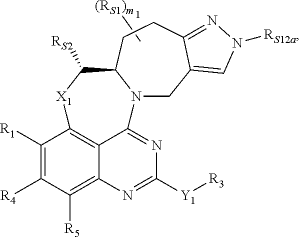

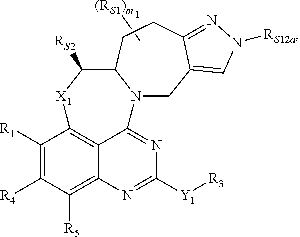

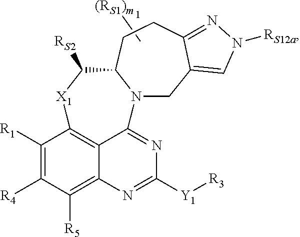

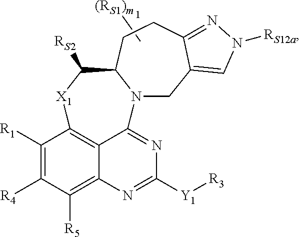

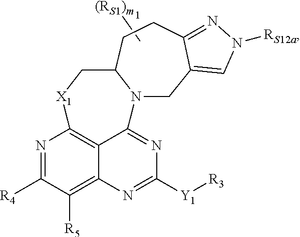

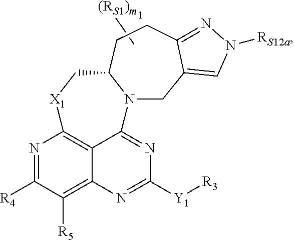

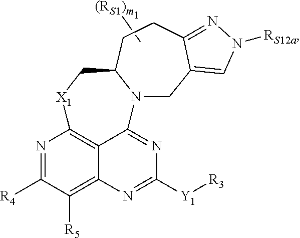

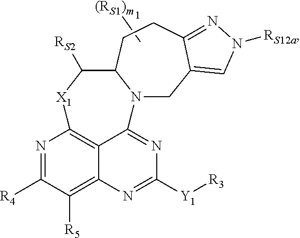

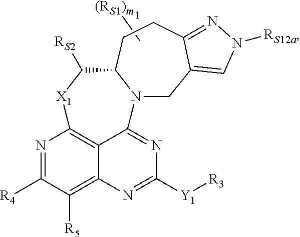

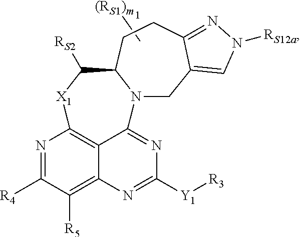

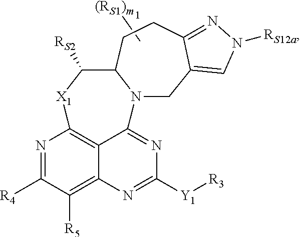

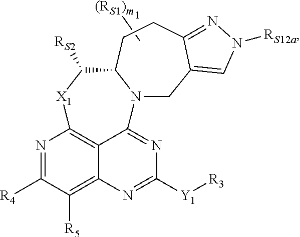

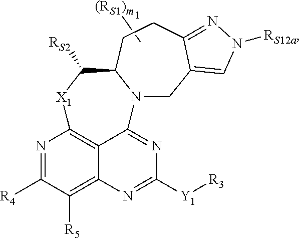

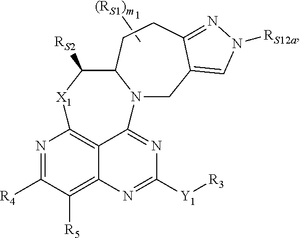

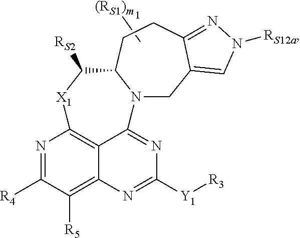

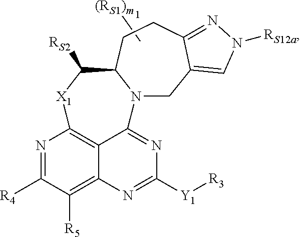

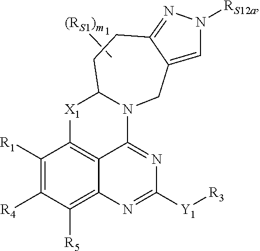

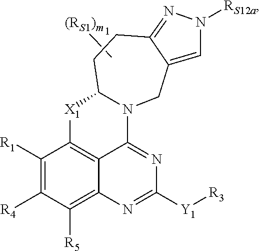

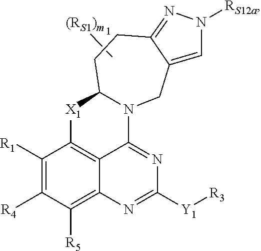

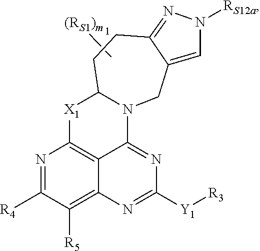

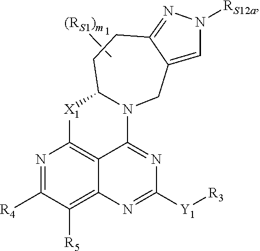

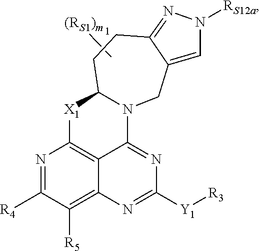

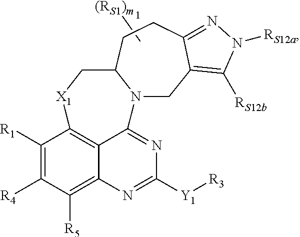

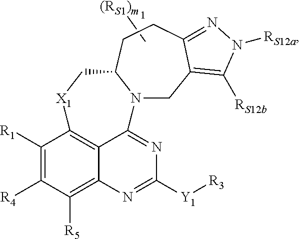

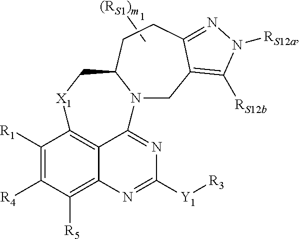

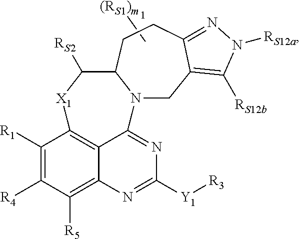

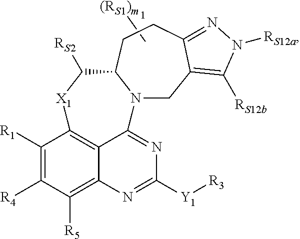

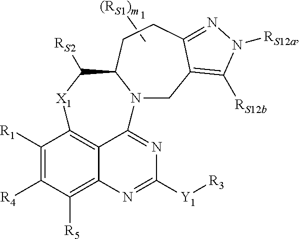

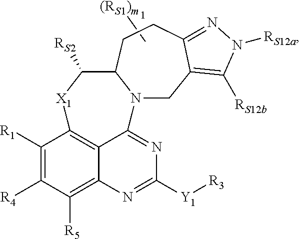

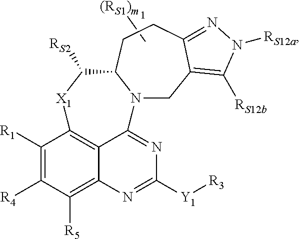

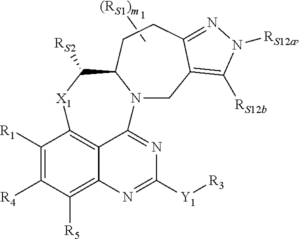

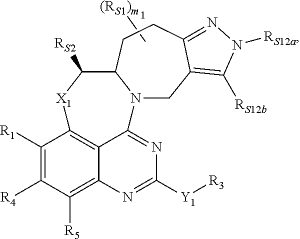

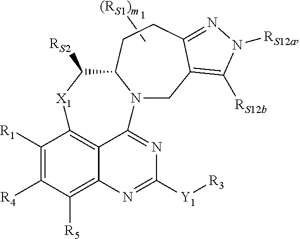

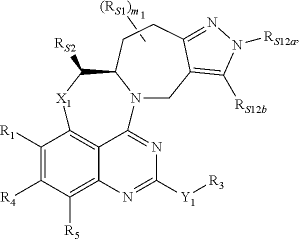

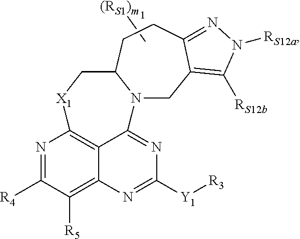

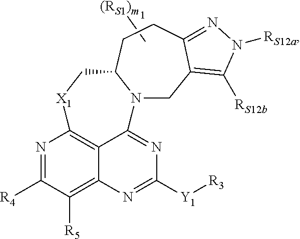

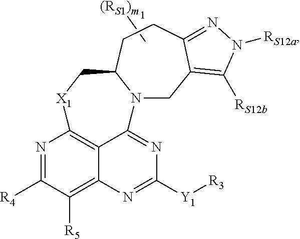

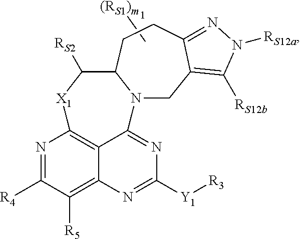

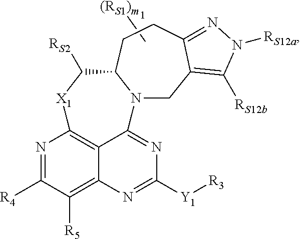

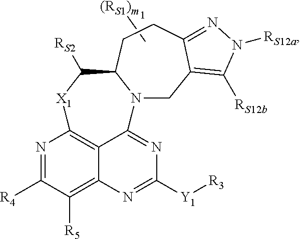

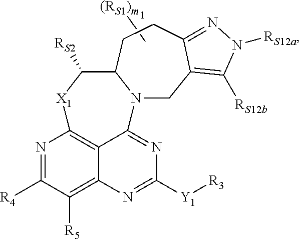

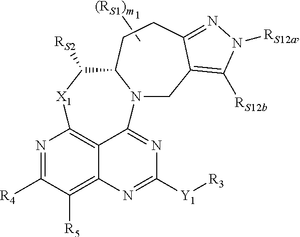

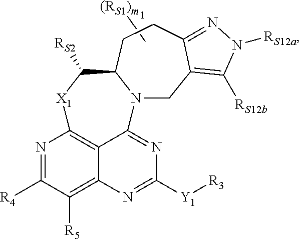

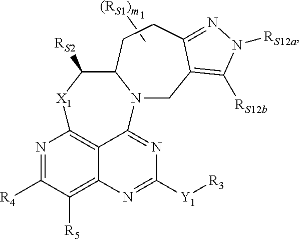

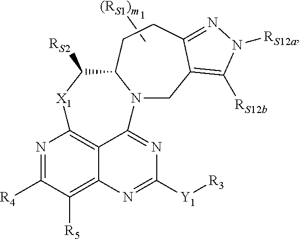

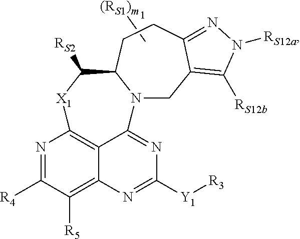

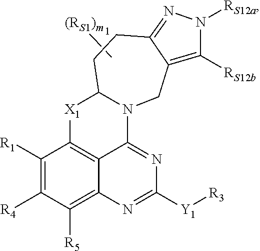

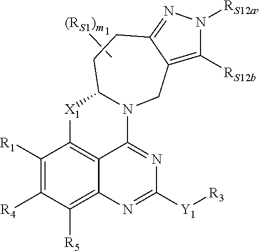

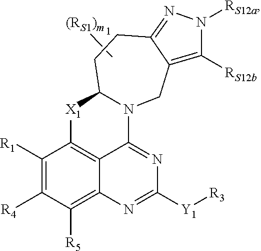

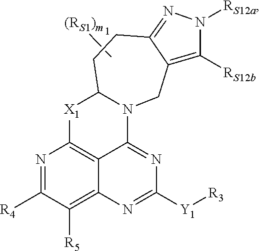

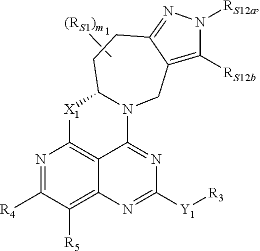

or

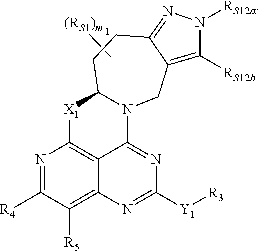

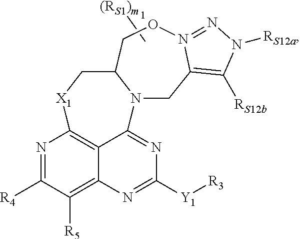

[18]. The compound of any one of [1] to [17], wherein, the compound is any one of the formulas in the Table B:

TABLE B

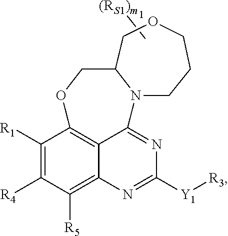

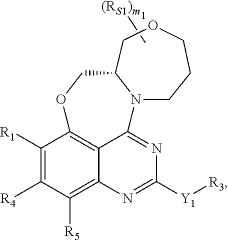

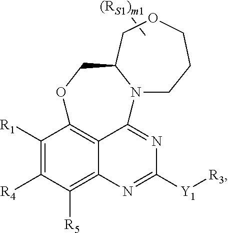

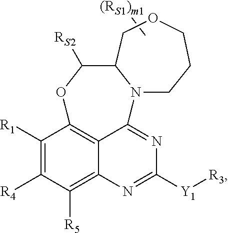

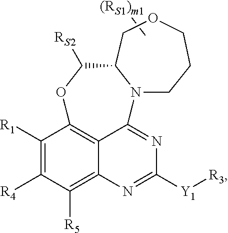

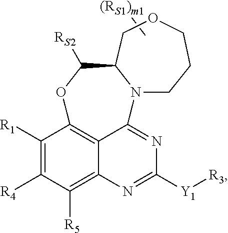

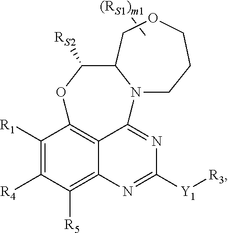

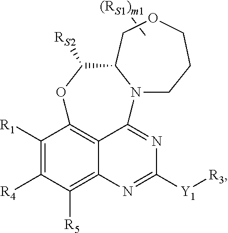

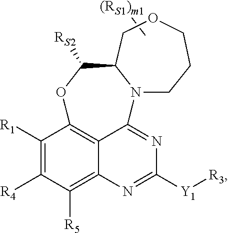

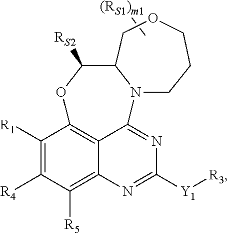

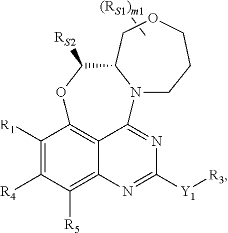

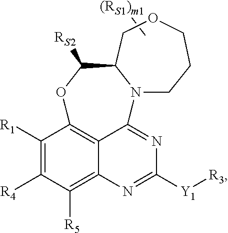

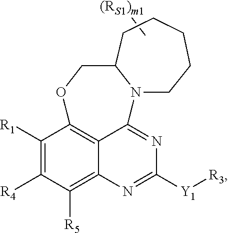

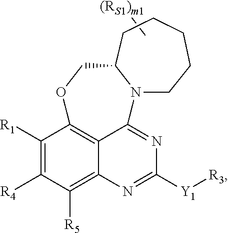

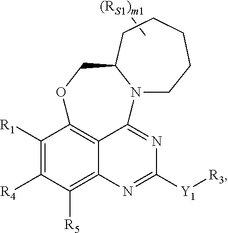

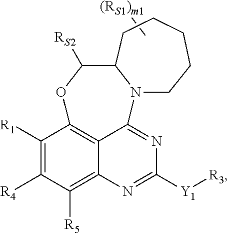

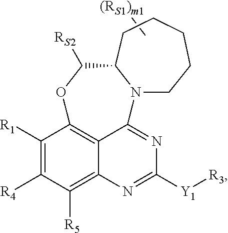

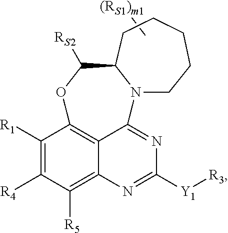

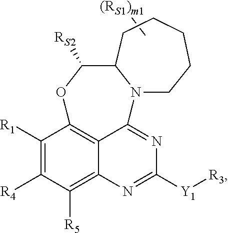

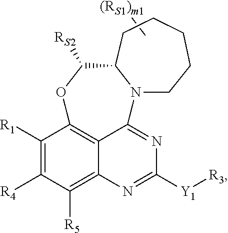

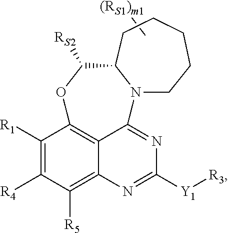

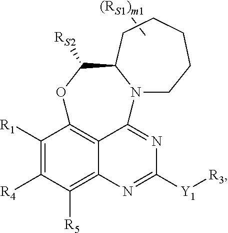

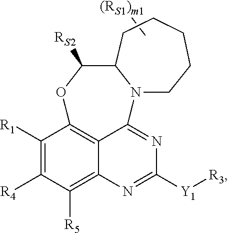

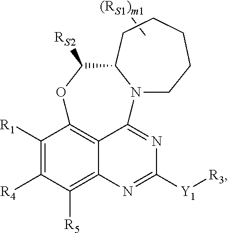

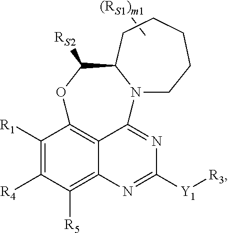

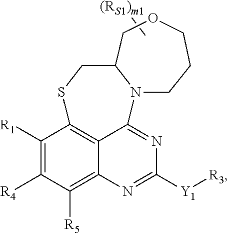

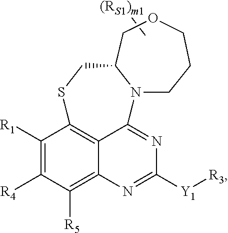

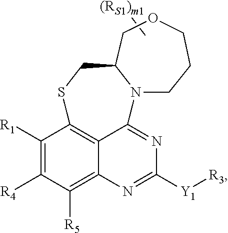

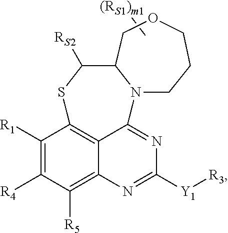

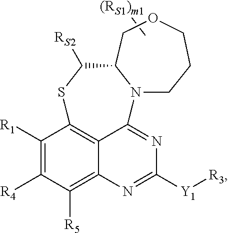

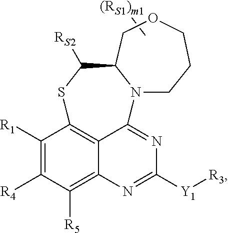

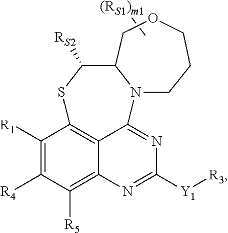

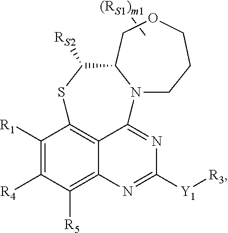

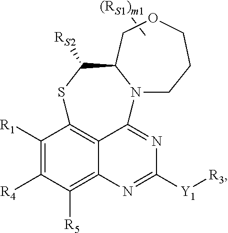

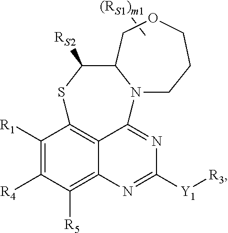

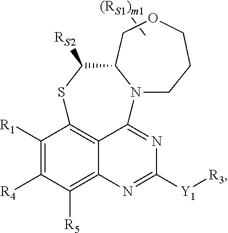

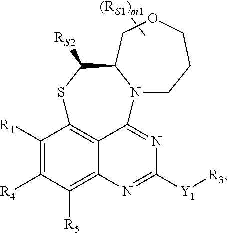

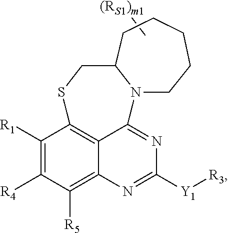

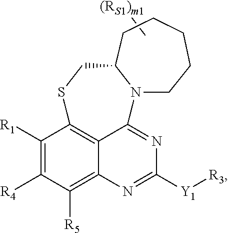

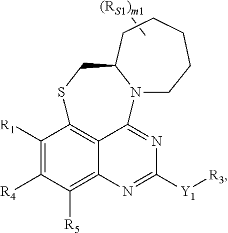

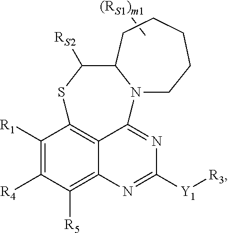

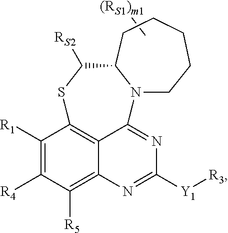

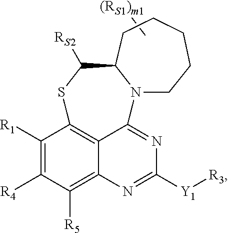

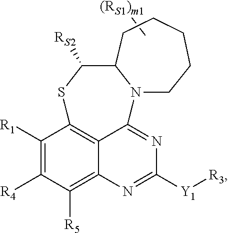

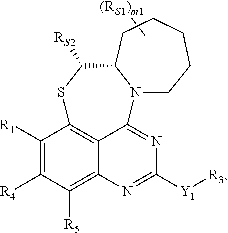

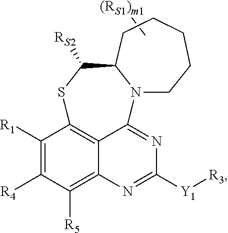

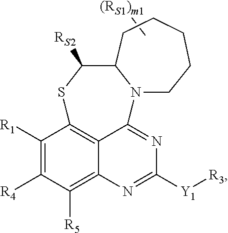

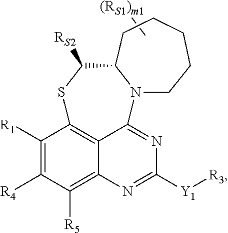

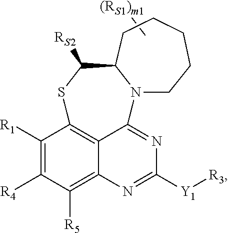

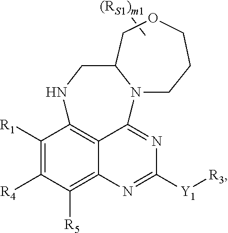

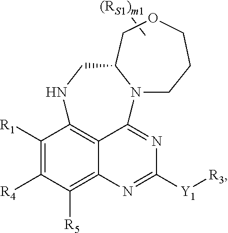

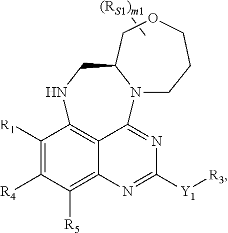

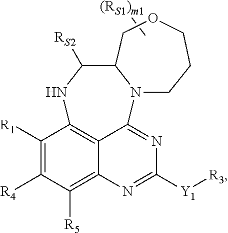

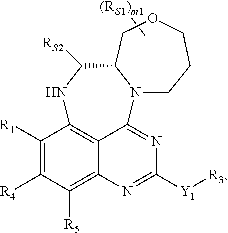

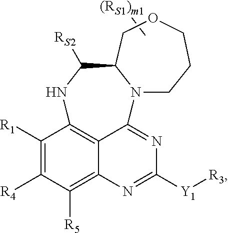

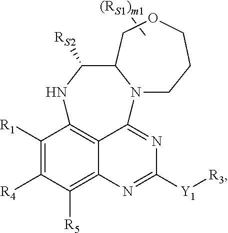

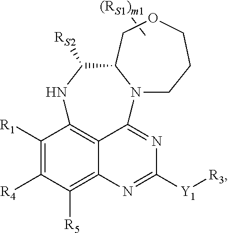

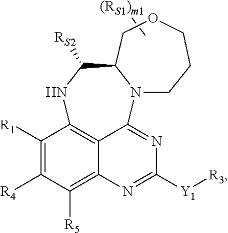

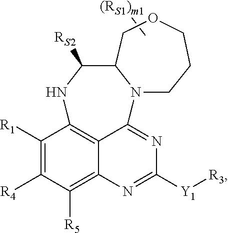

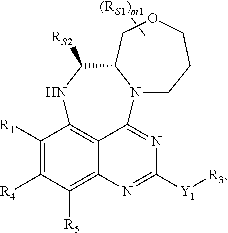

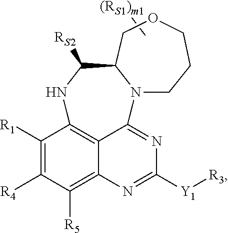

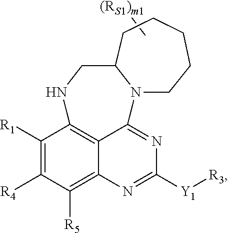

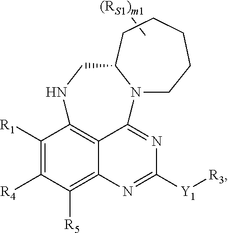

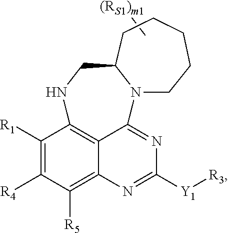

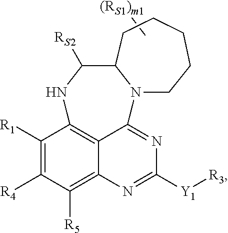

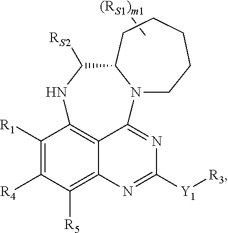

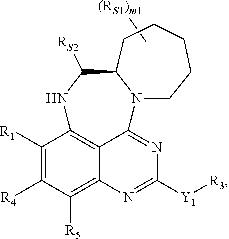

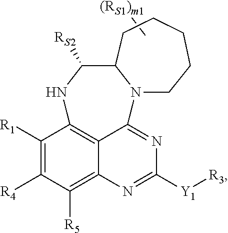

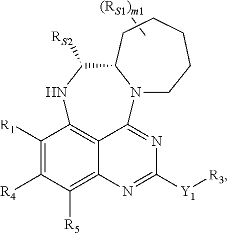

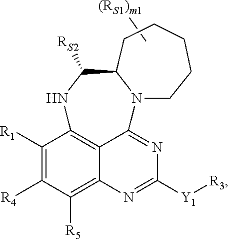

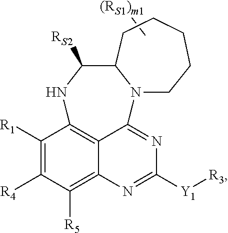

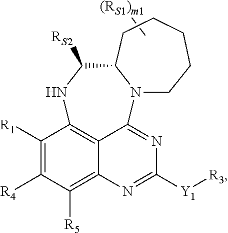

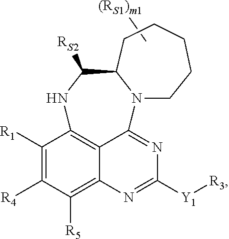

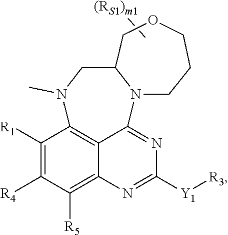

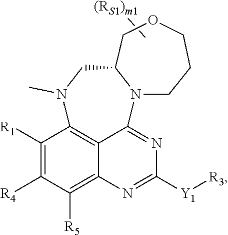

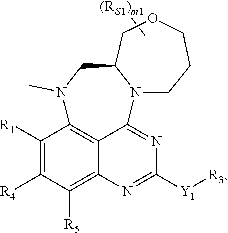

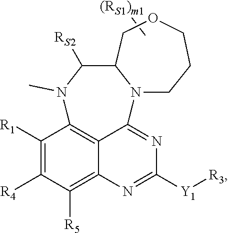

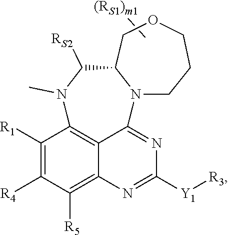

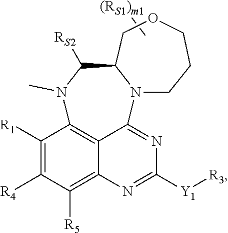

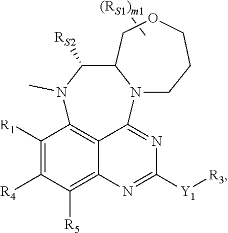

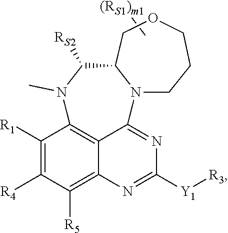

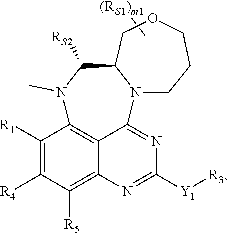

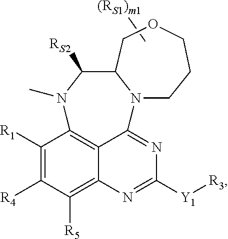

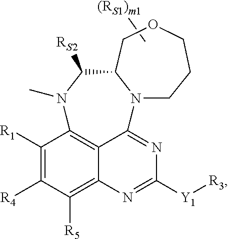

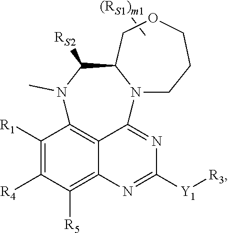

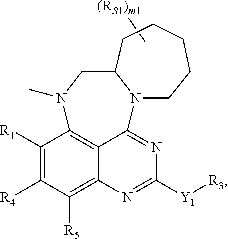

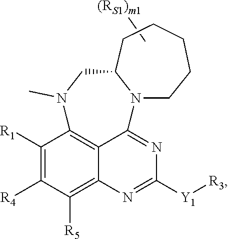

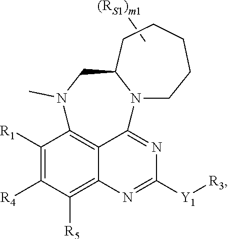

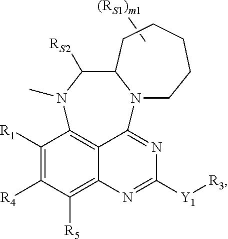

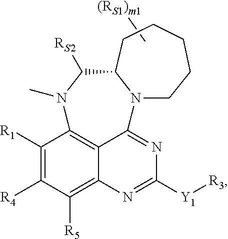

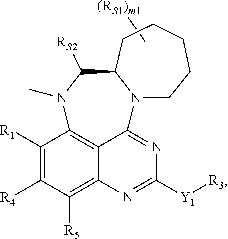

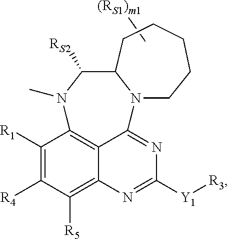

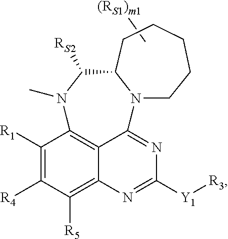

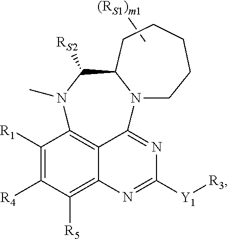

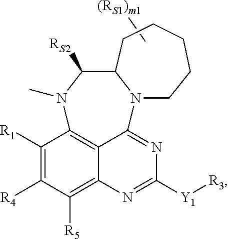

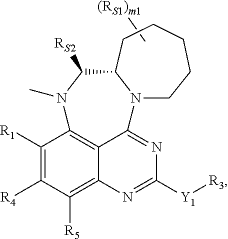

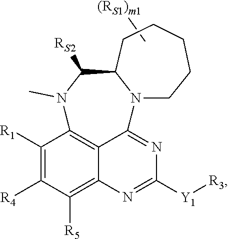

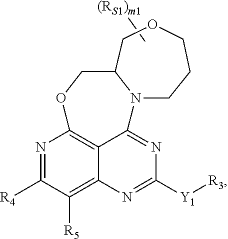

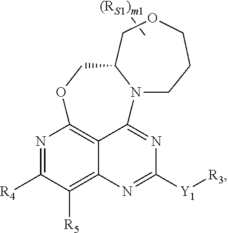

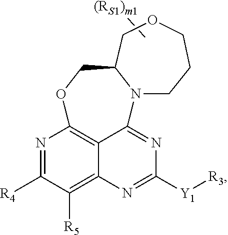

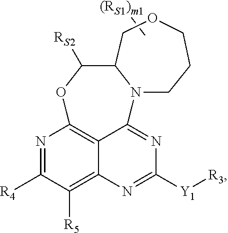

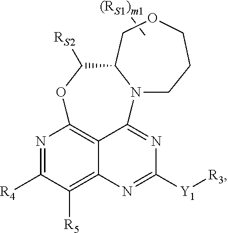

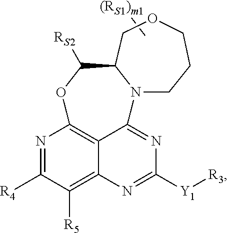

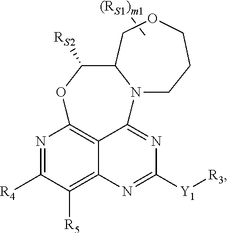

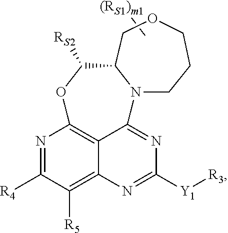

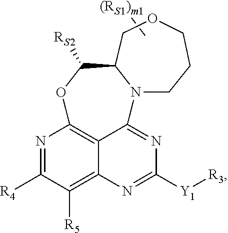

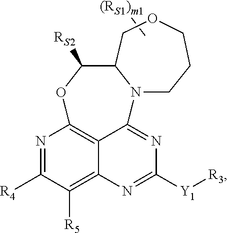

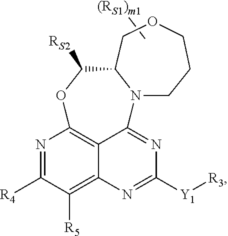

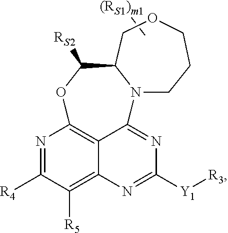

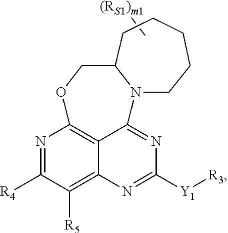

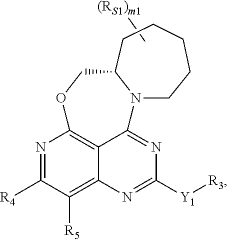

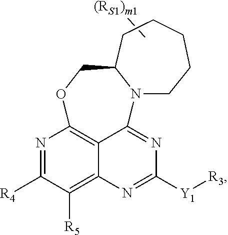

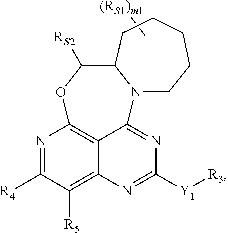

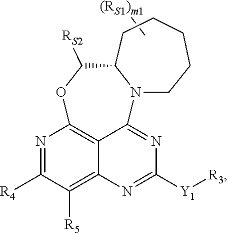

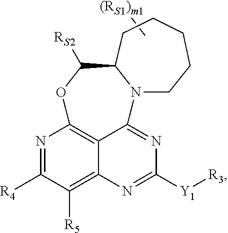

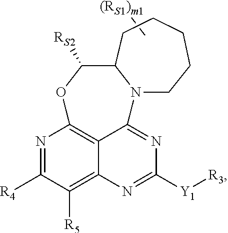

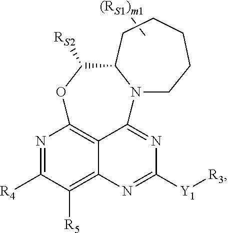

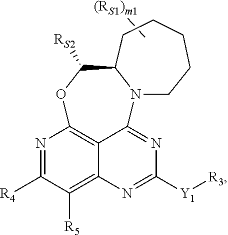

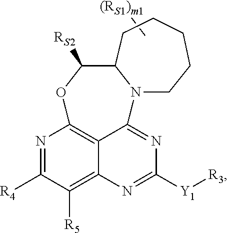

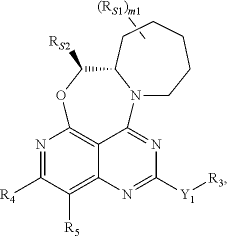

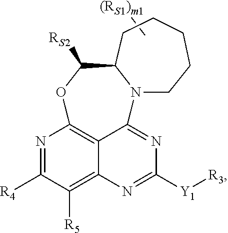

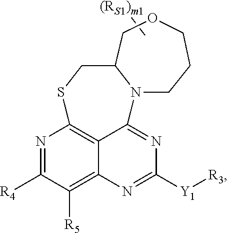

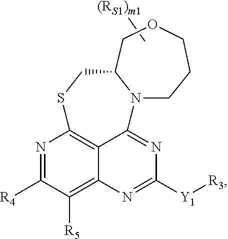

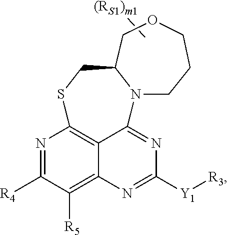

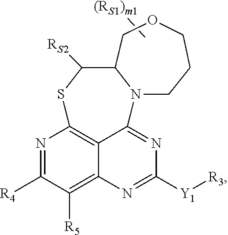

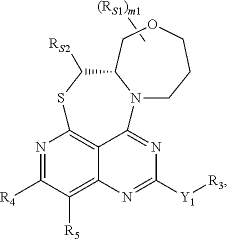

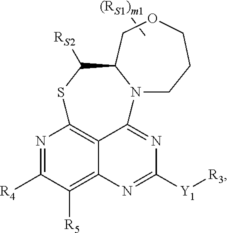

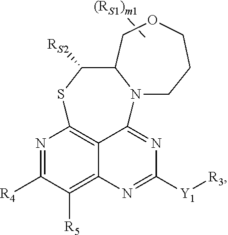

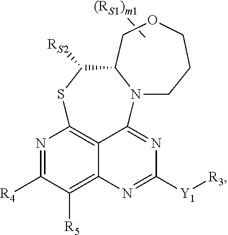

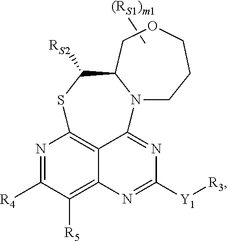

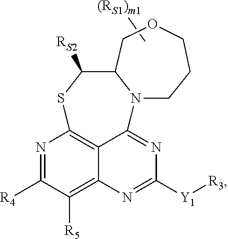

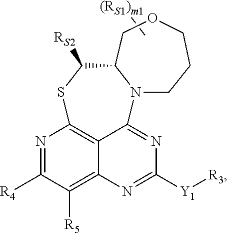

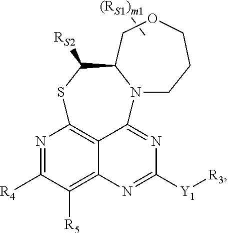

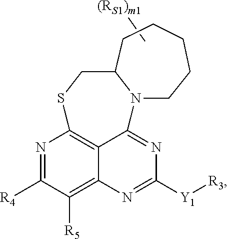

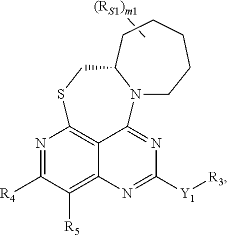

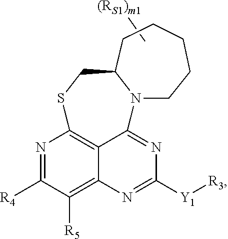

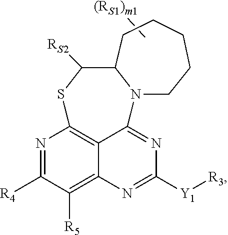

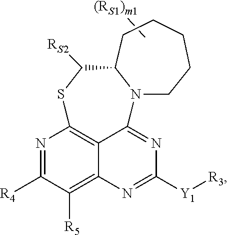

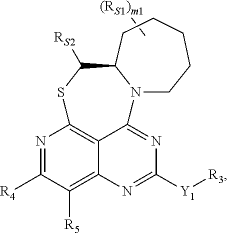

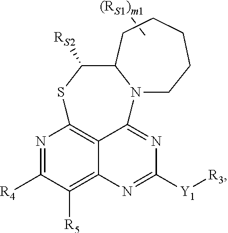

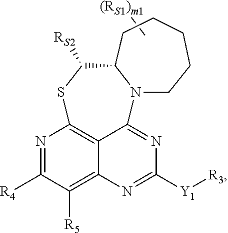

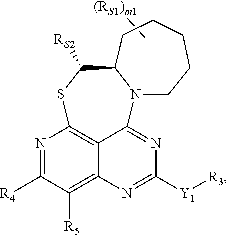

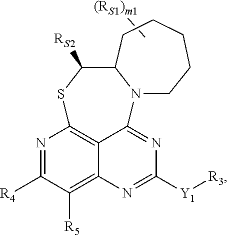

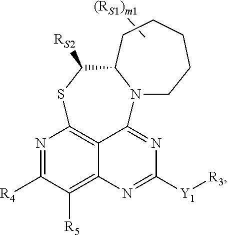

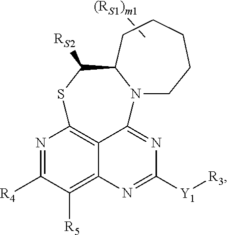

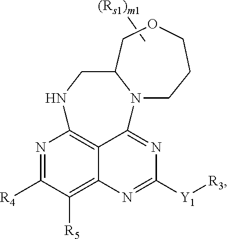

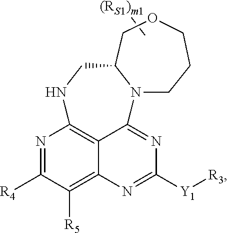

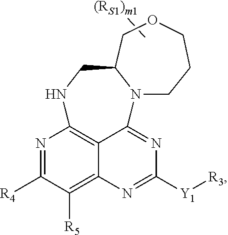

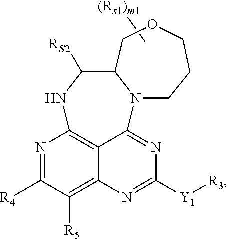

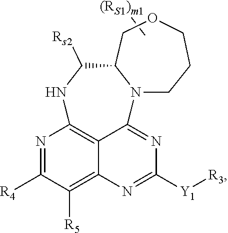

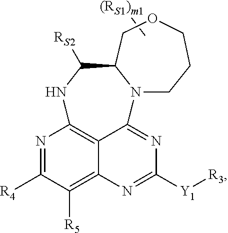

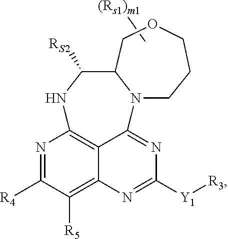

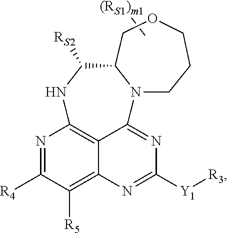

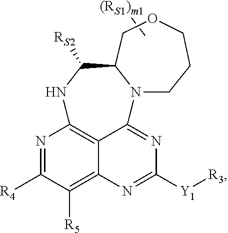

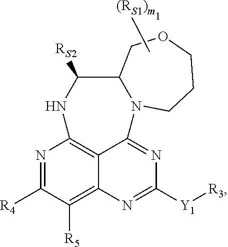

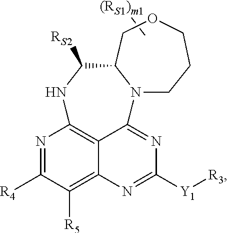

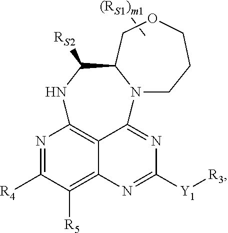

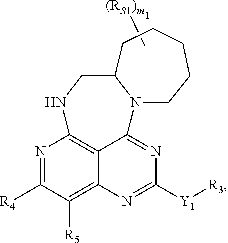

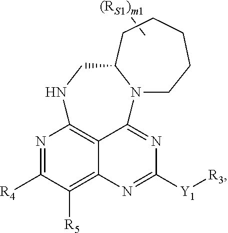

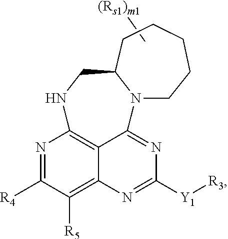

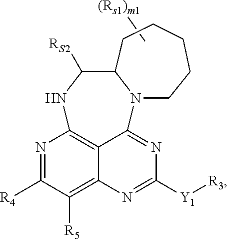

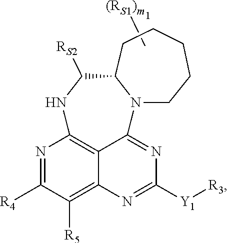

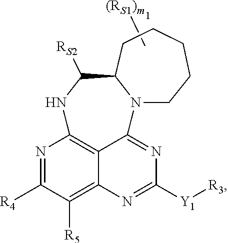

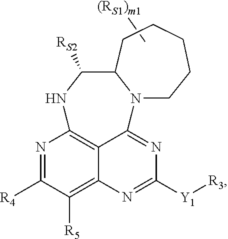

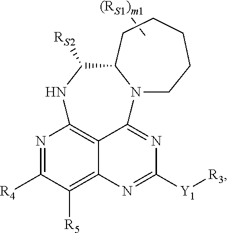

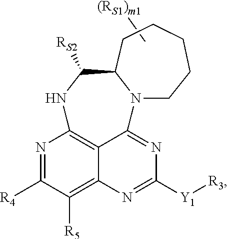

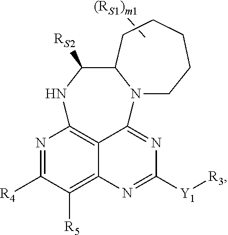

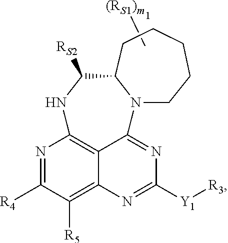

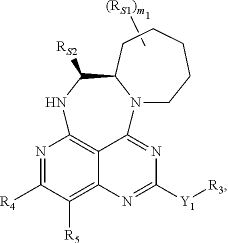

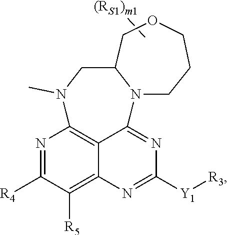

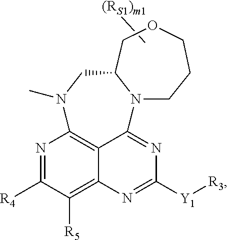

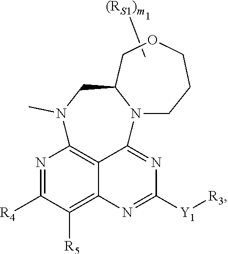

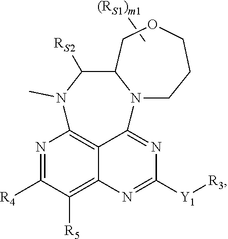

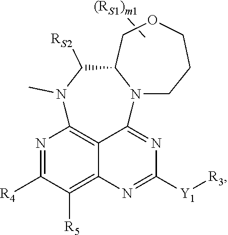

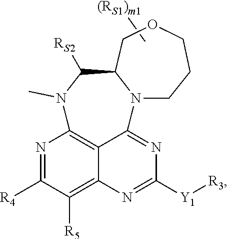

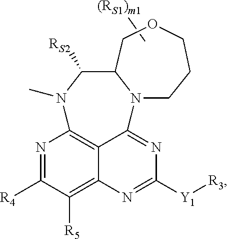

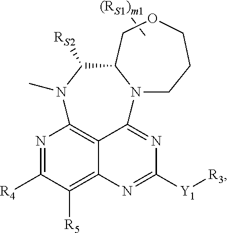

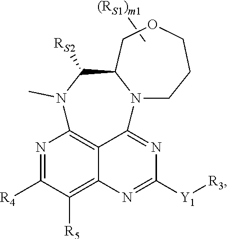

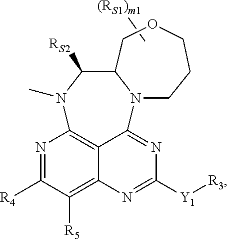

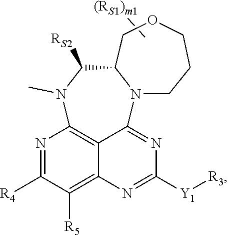

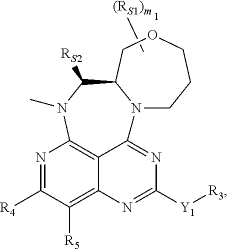

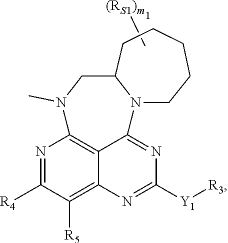

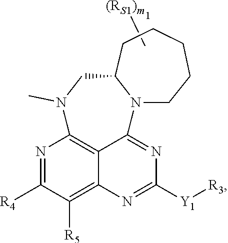

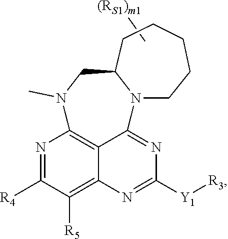

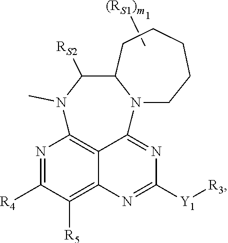

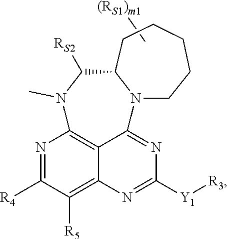

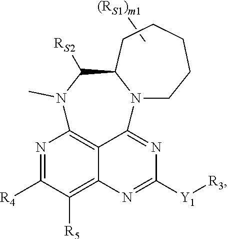

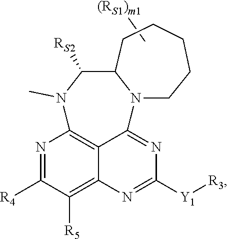

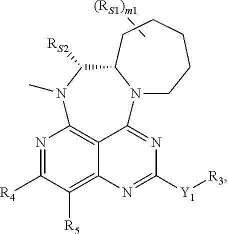

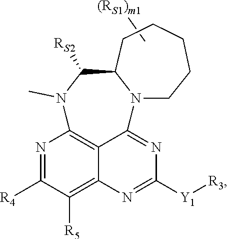

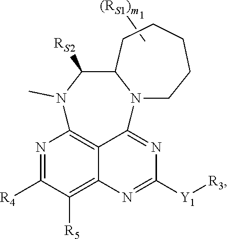

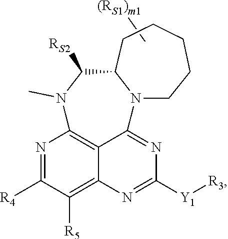

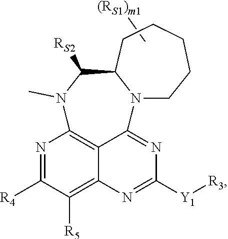

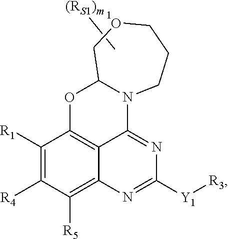

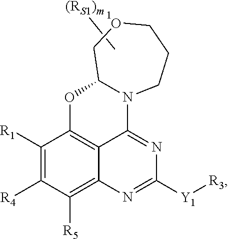

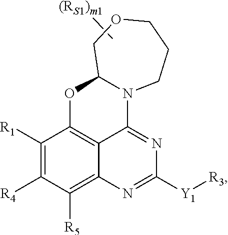

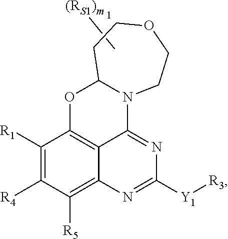

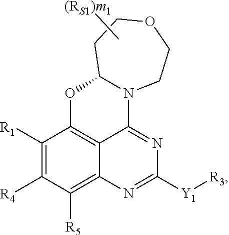

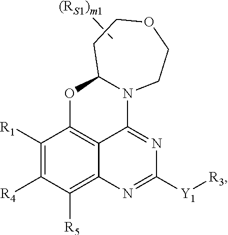

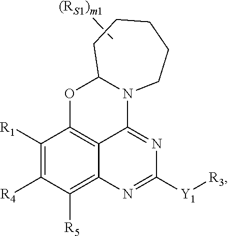

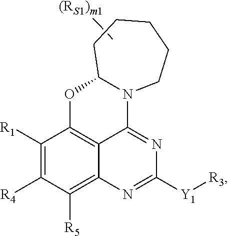

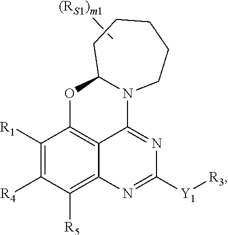

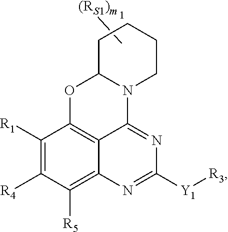

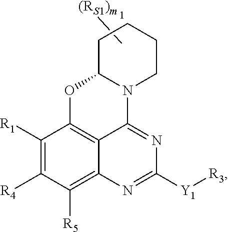

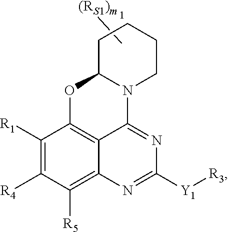

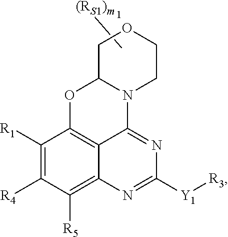

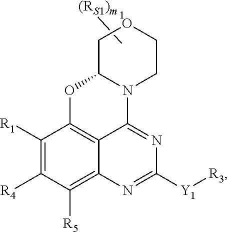

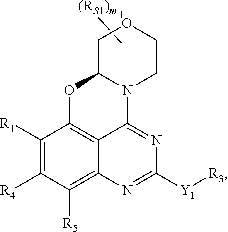

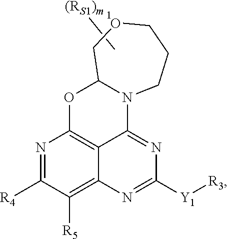

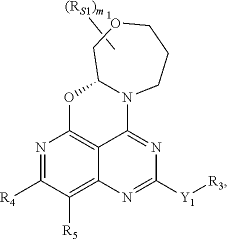

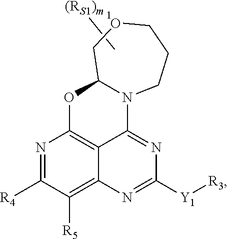

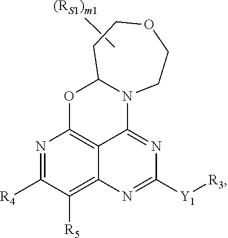

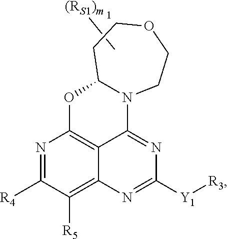

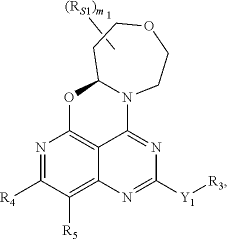

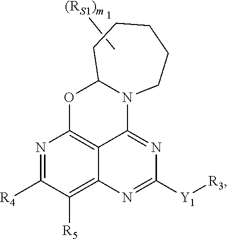

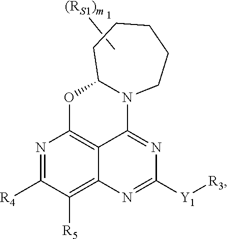

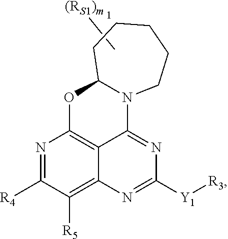

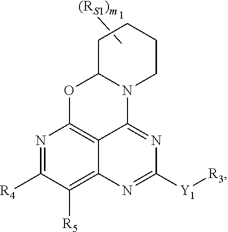

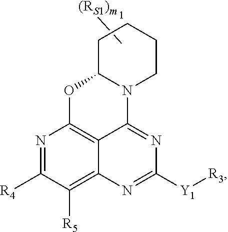

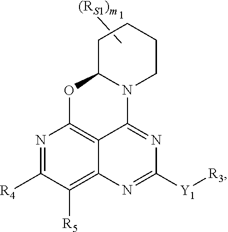

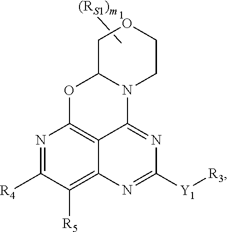

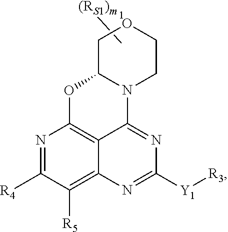

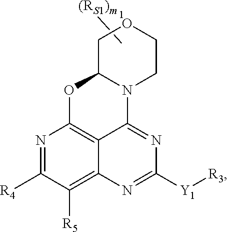

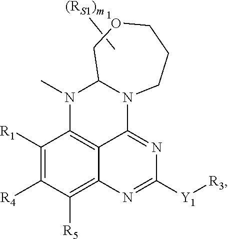

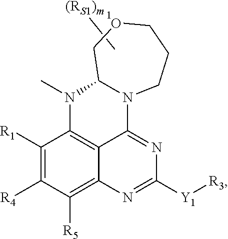

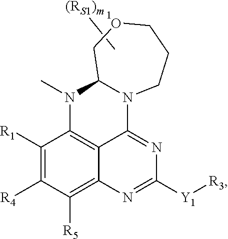

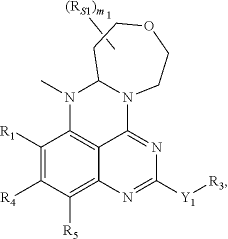

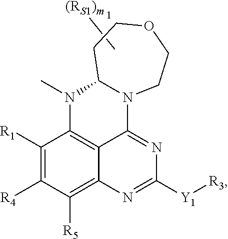

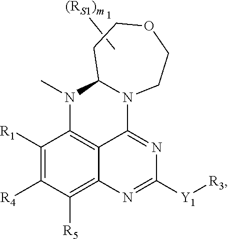

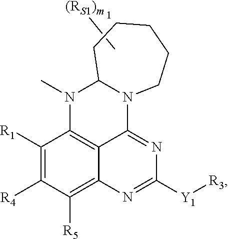

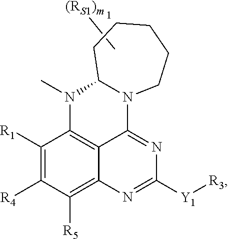

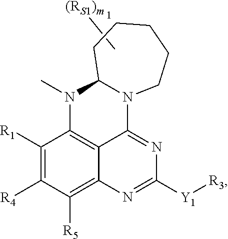

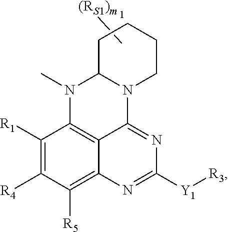

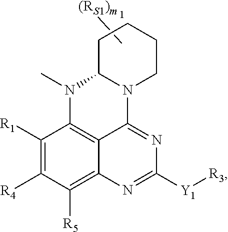

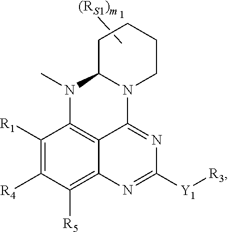

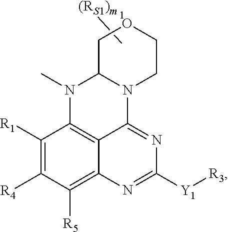

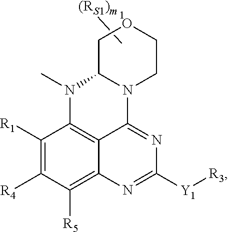

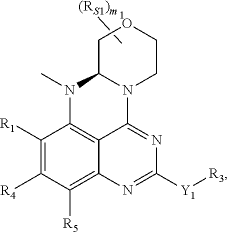

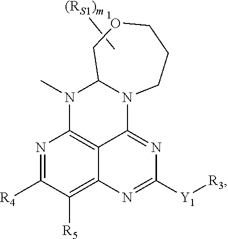

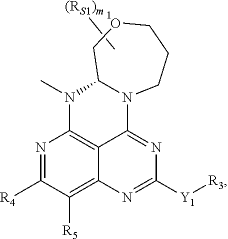

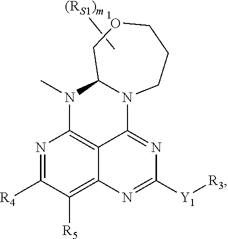

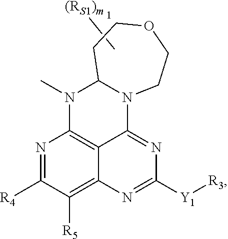

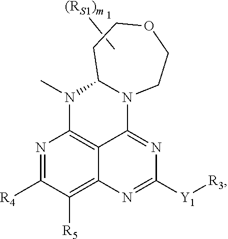

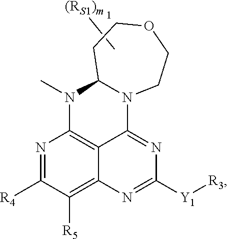

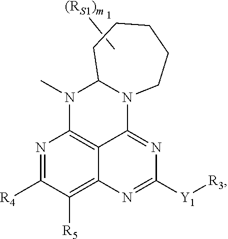

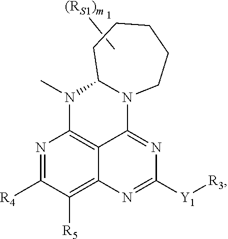

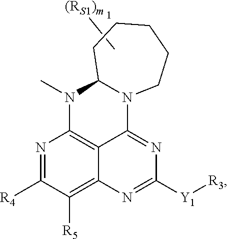

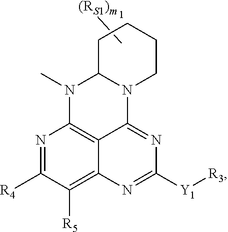

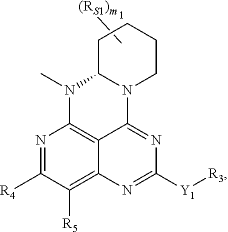

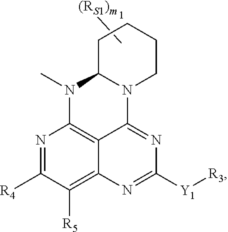

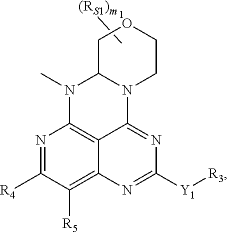

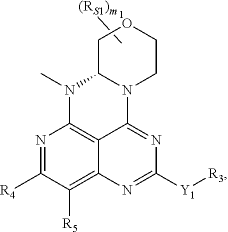

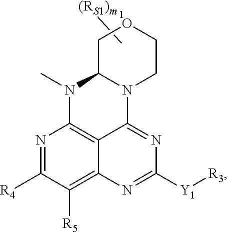

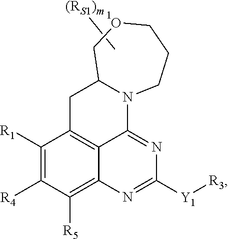

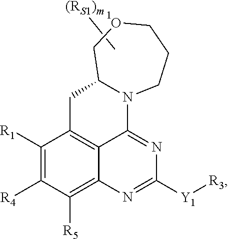

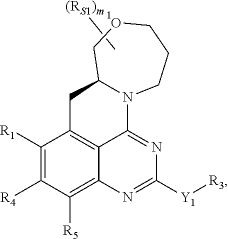

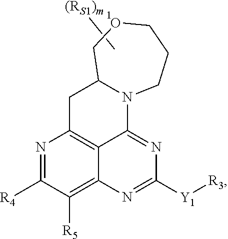

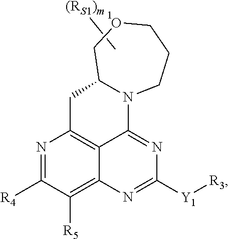

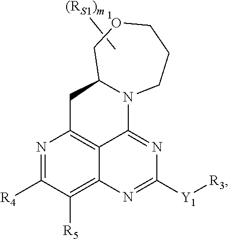

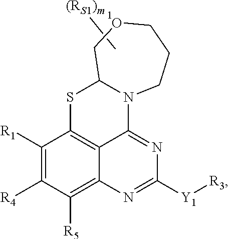

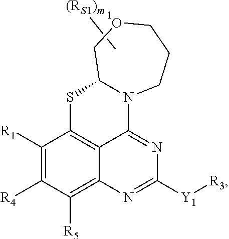

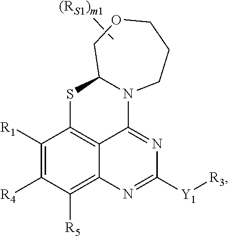

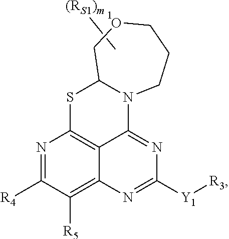

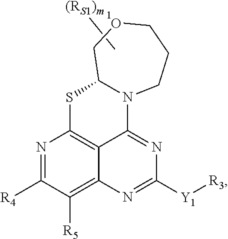

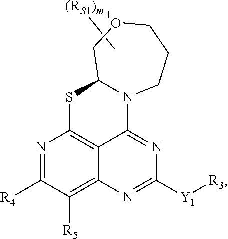

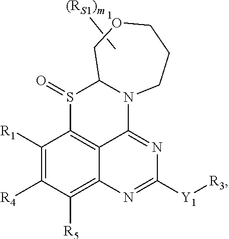

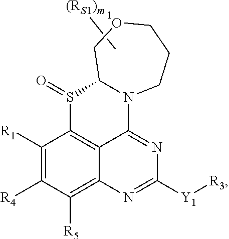

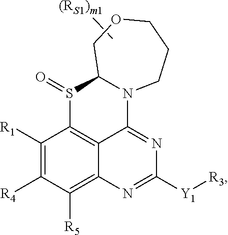

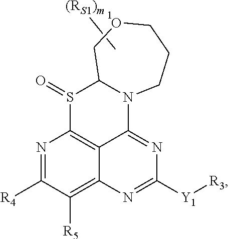

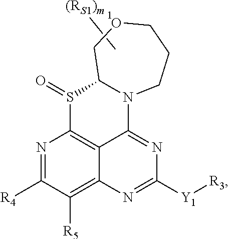

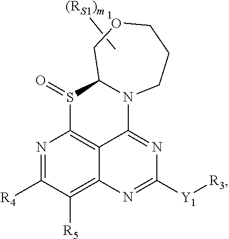

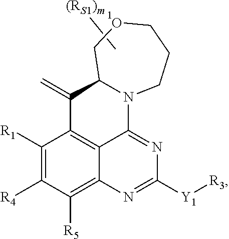

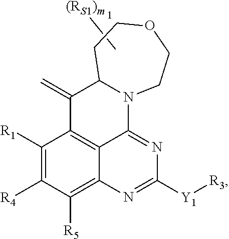

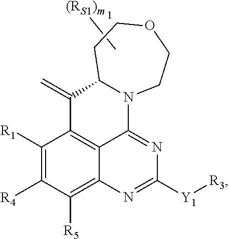

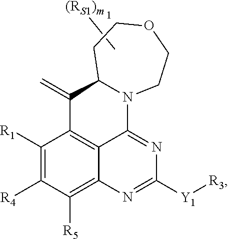

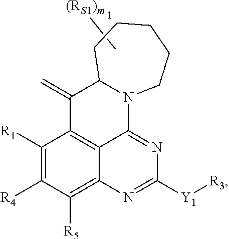

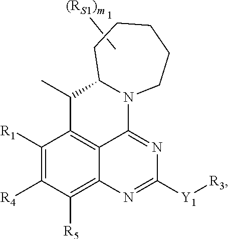

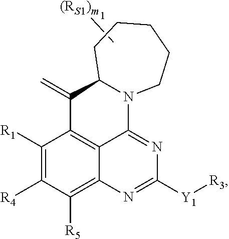

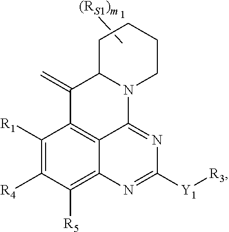

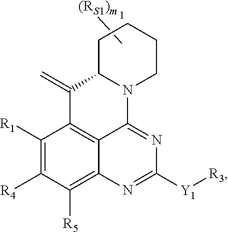

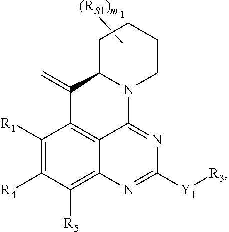

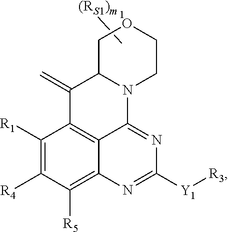

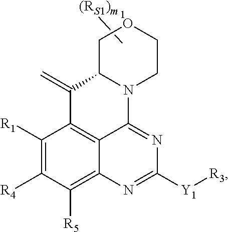

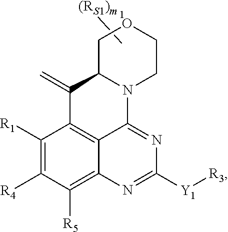

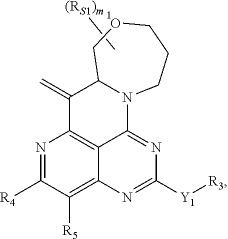

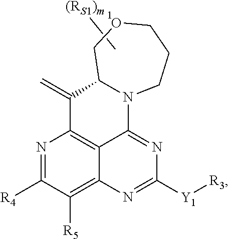

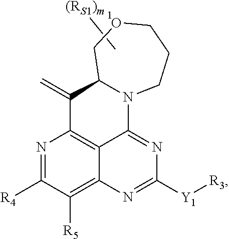

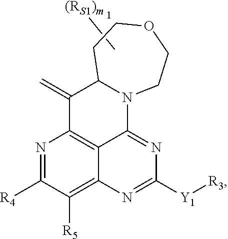

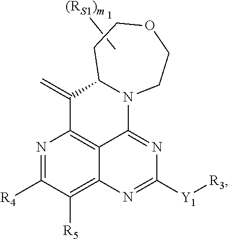

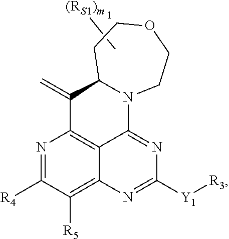

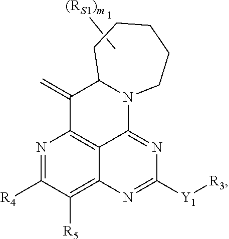

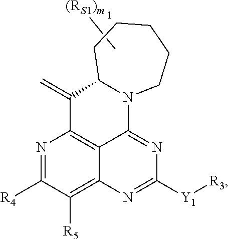

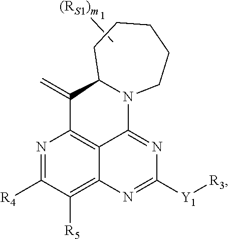

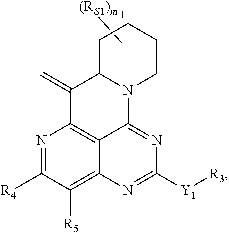

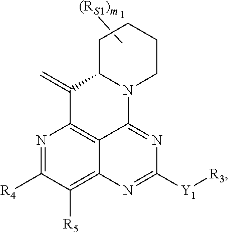

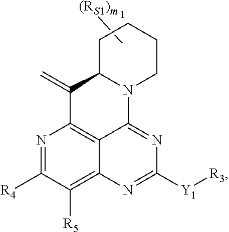

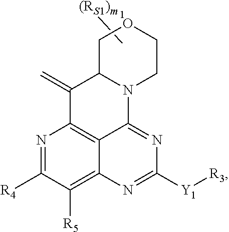

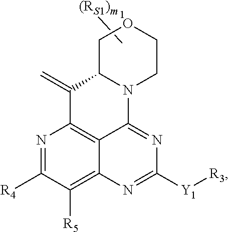

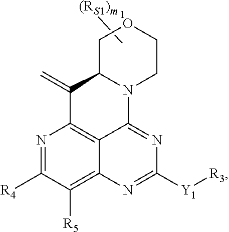

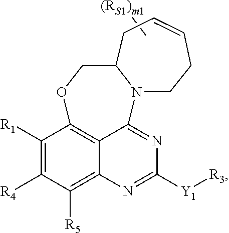

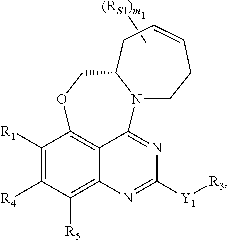

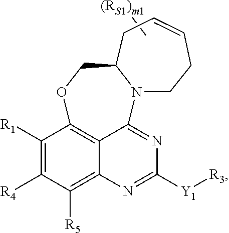

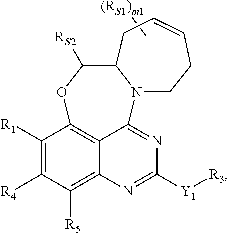

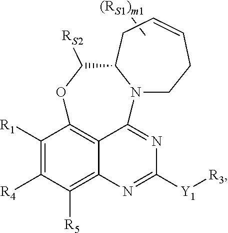

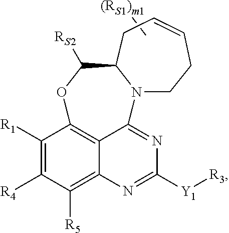

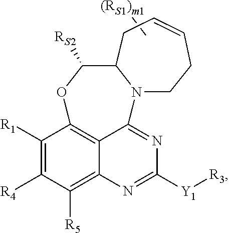

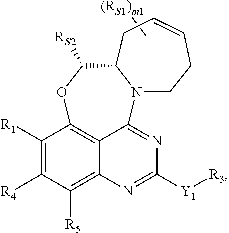

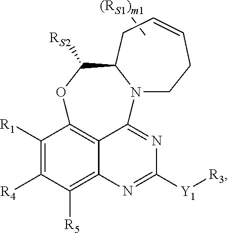

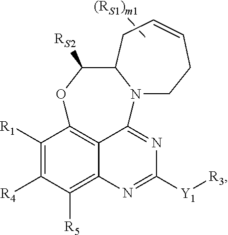

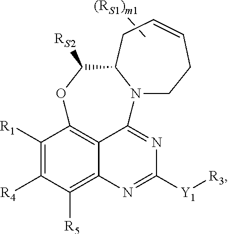

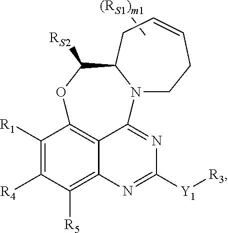

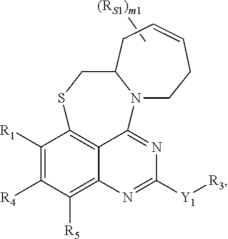

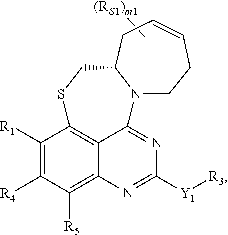

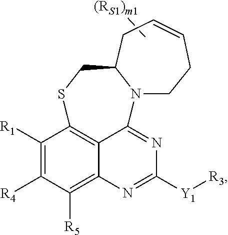

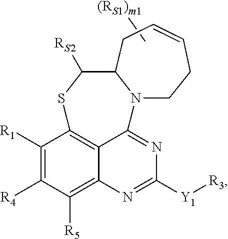

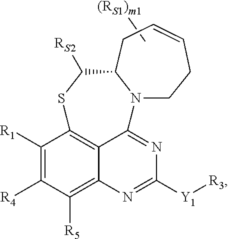

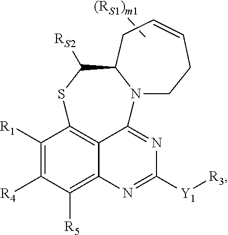

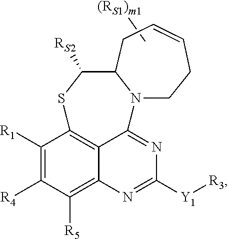

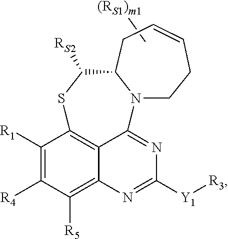

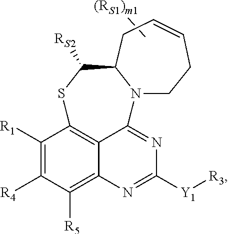

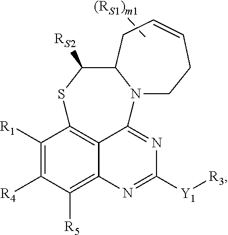

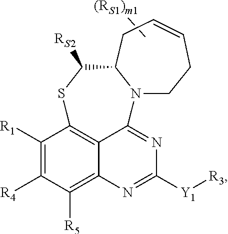

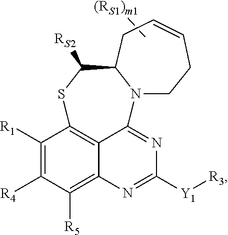

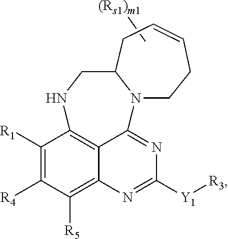

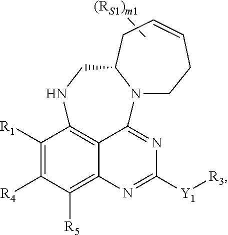

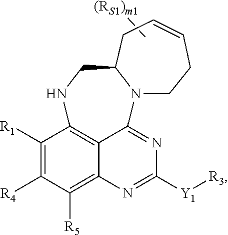

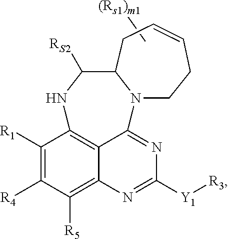

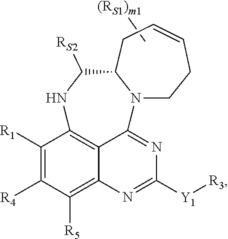

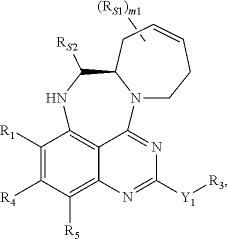

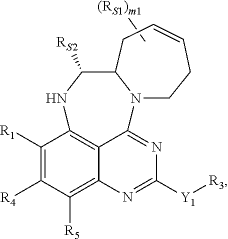

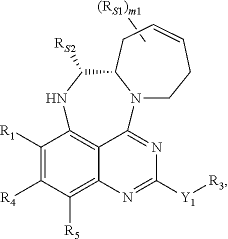

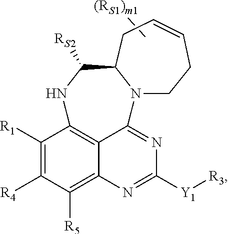

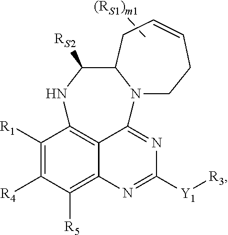

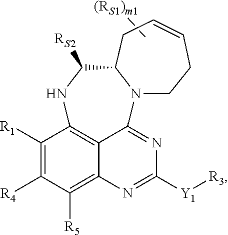

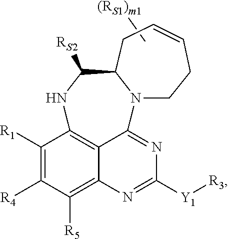

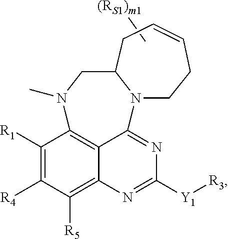

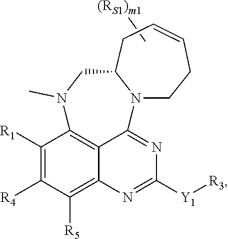

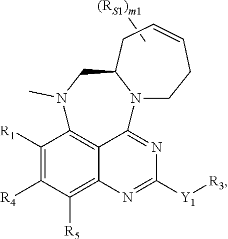

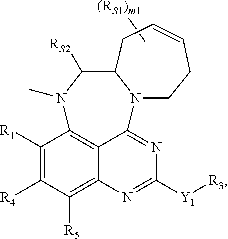

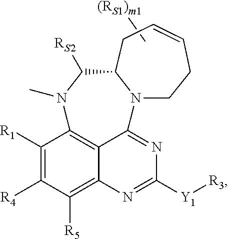

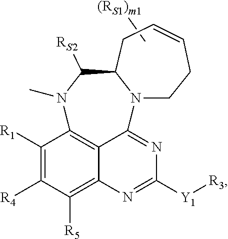

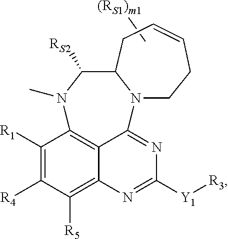

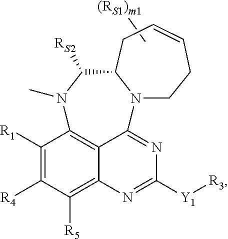

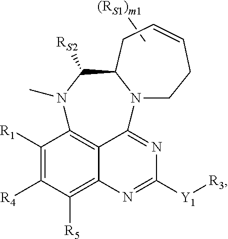

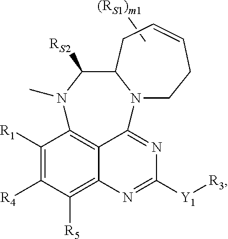

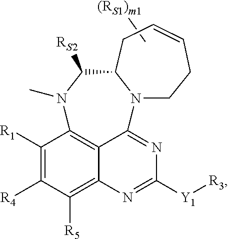

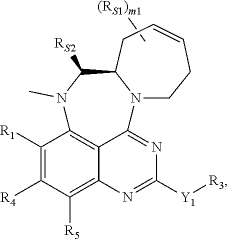

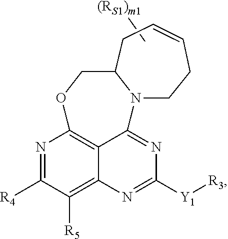

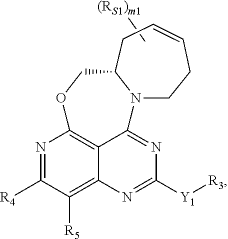

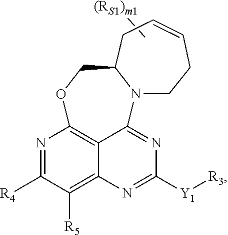

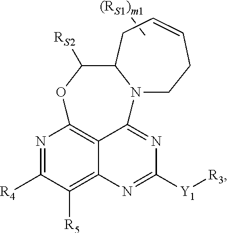

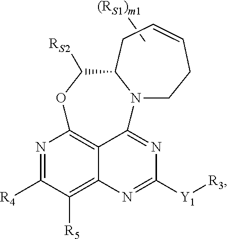

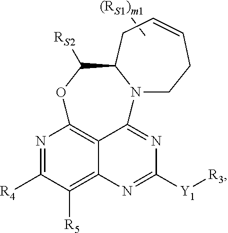

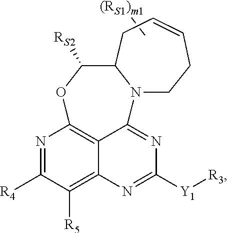

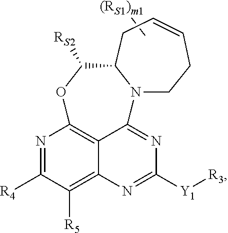

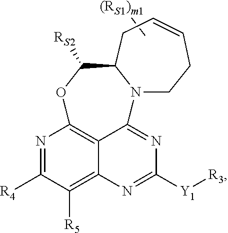

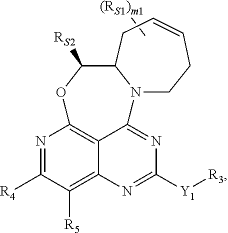

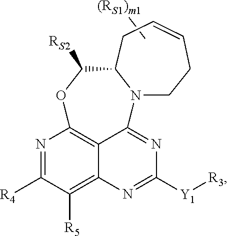

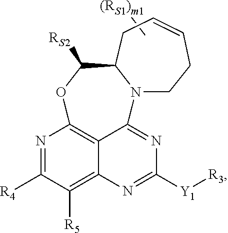

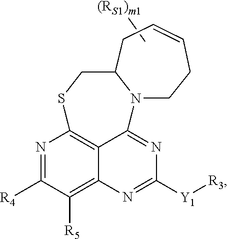

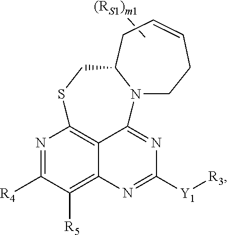

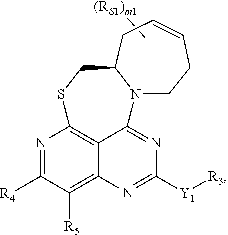

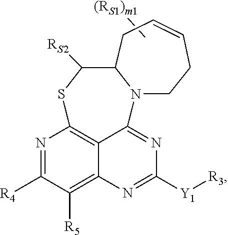

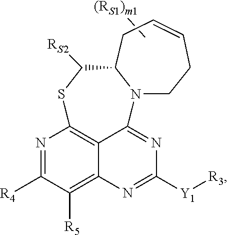

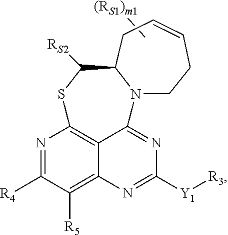

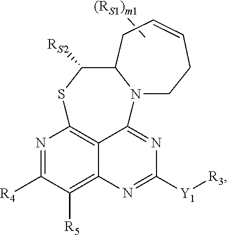

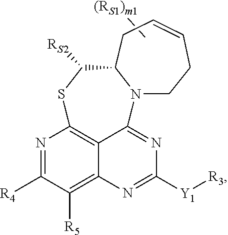

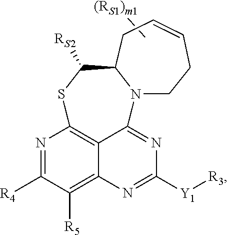

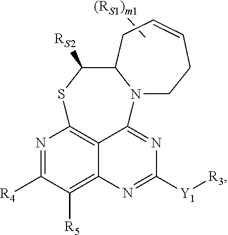

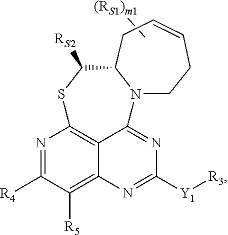

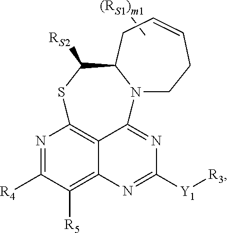

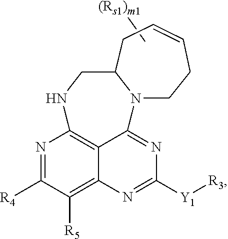

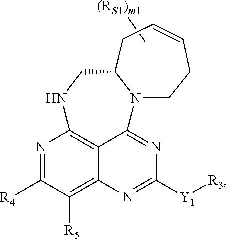

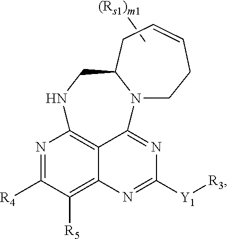

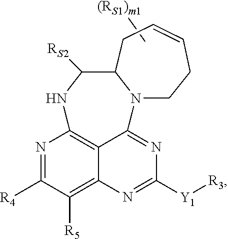

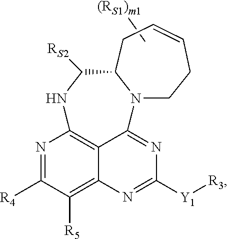

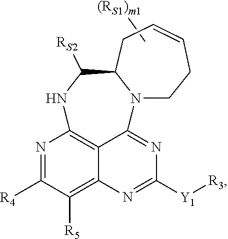

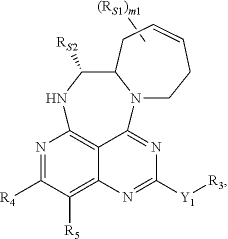

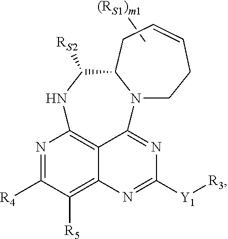

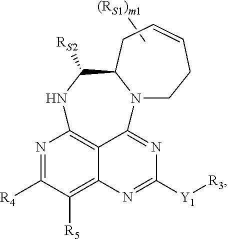

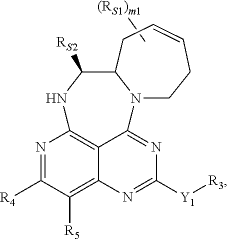

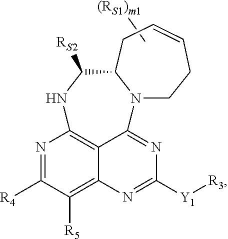

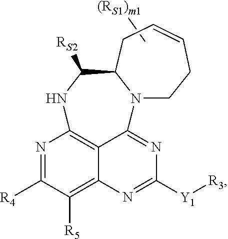

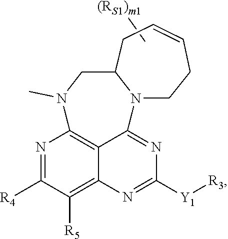

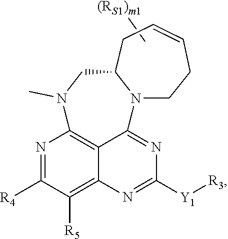

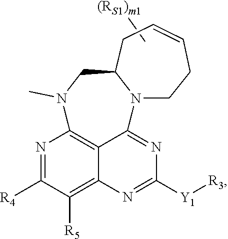

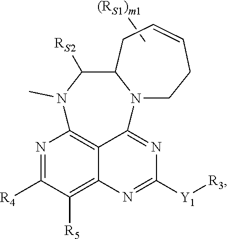

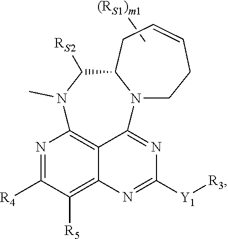

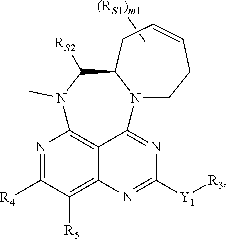

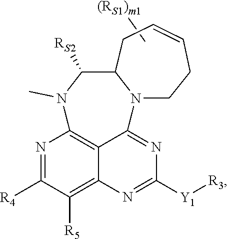

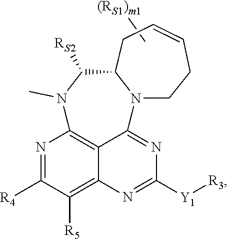

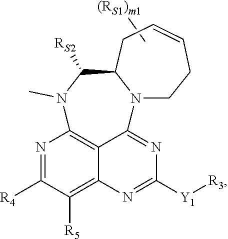

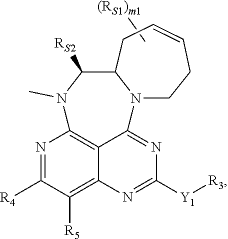

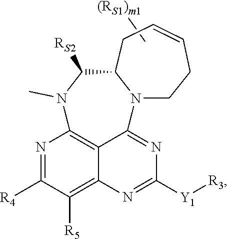

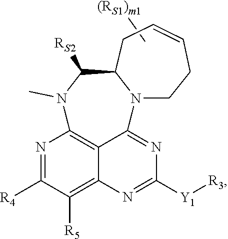

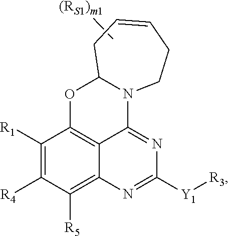

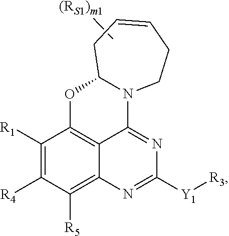

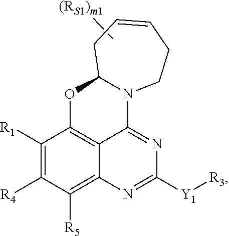

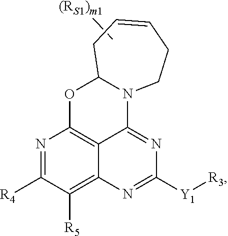

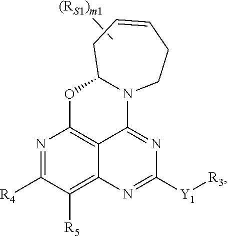

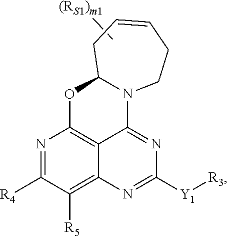

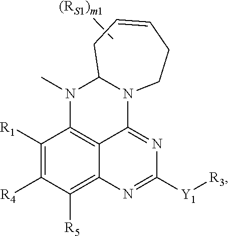

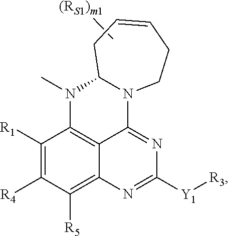

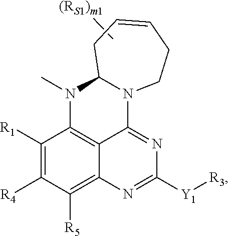

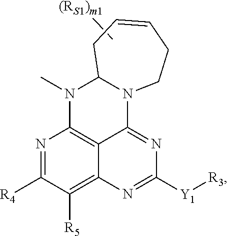

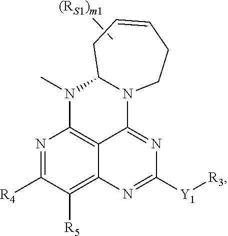

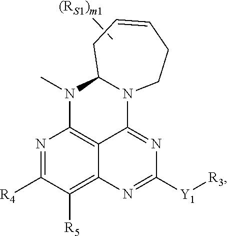

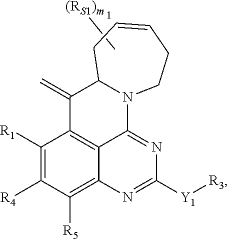

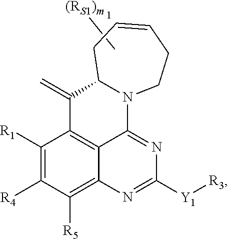

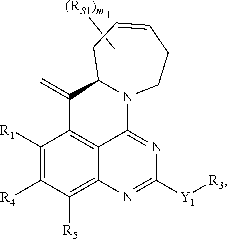

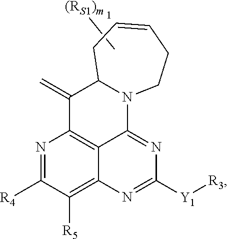

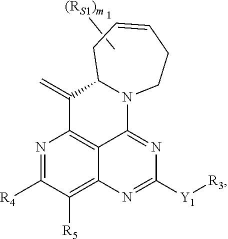

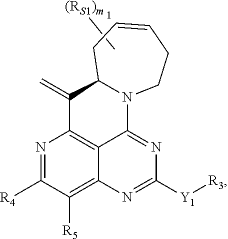

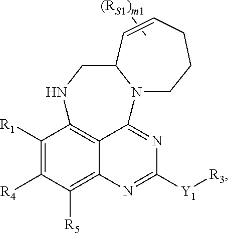

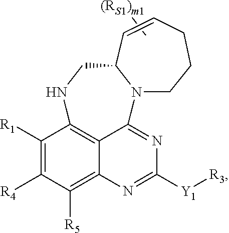

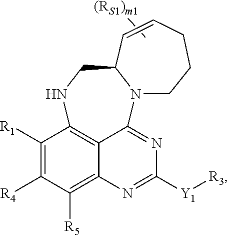

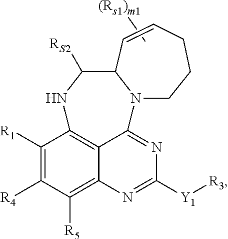

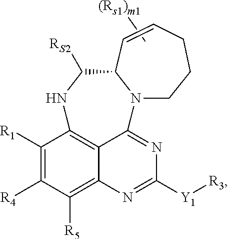

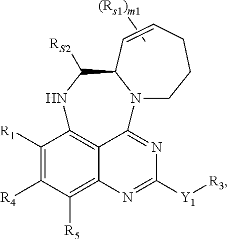

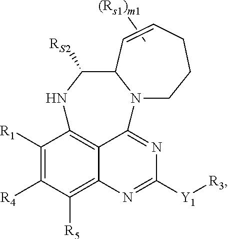

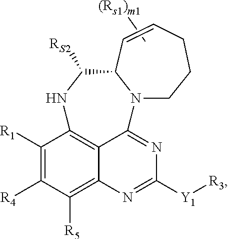

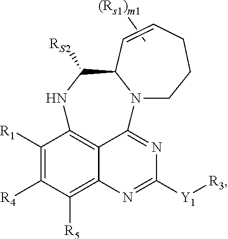

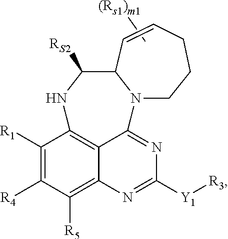

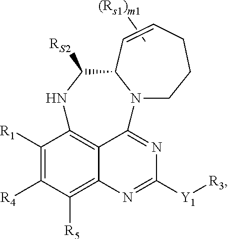

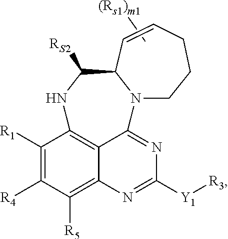

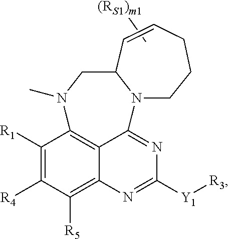

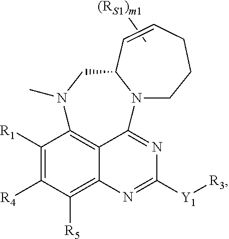

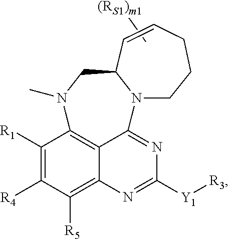

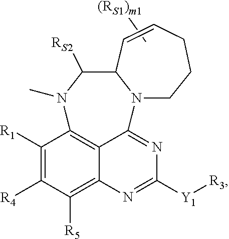

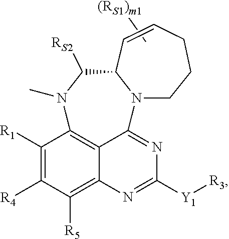

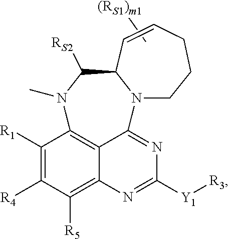

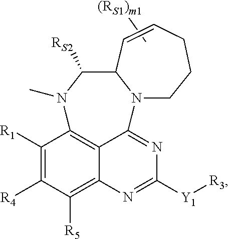

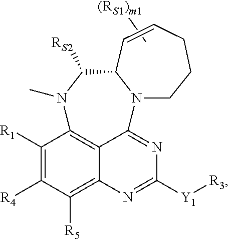

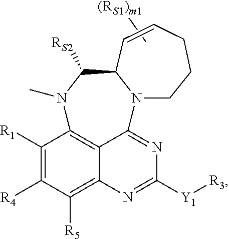

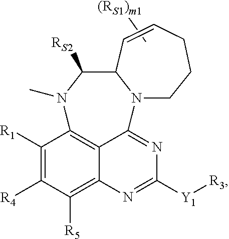

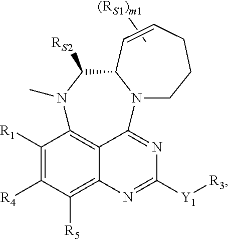

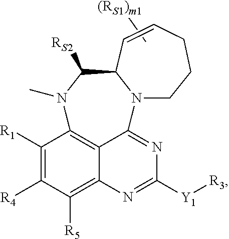

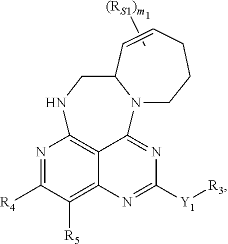

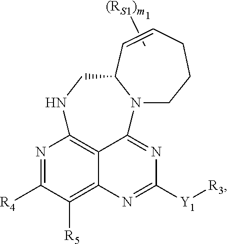

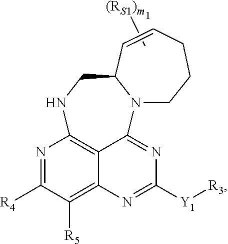

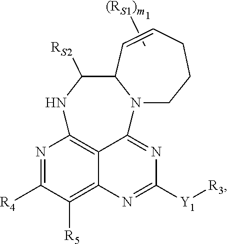

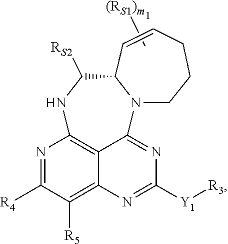

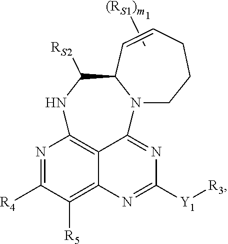

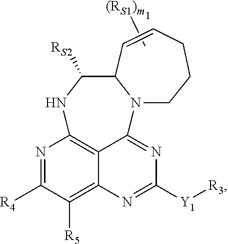

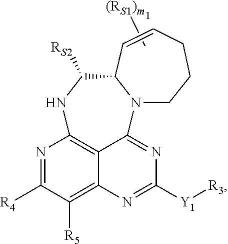

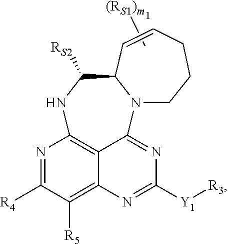

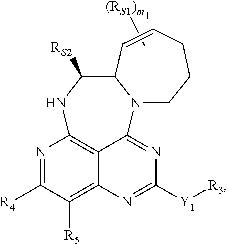

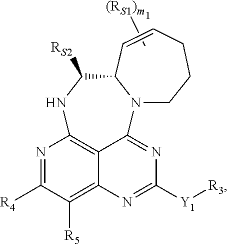

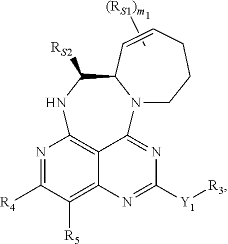

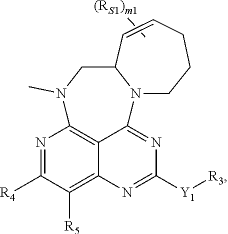

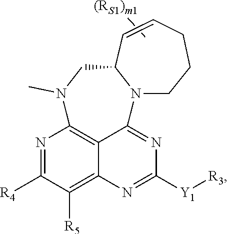

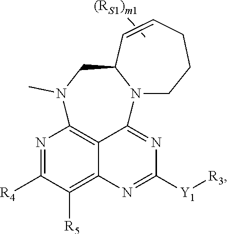

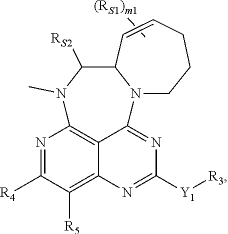

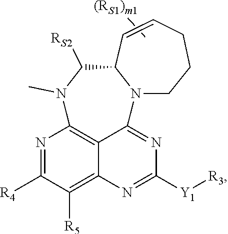

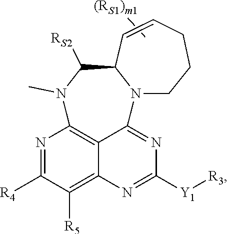

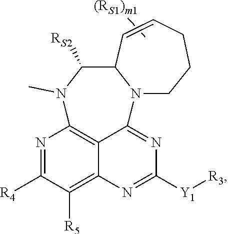

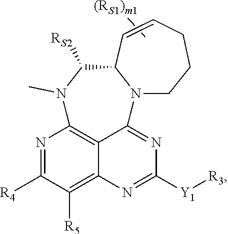

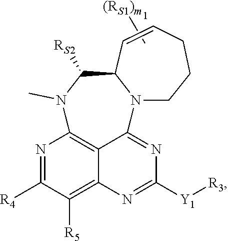

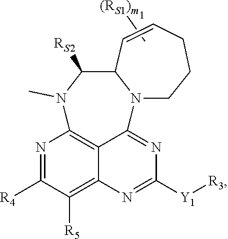

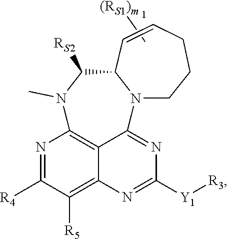

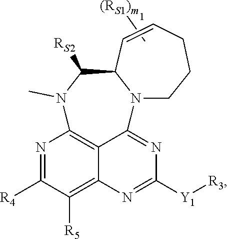

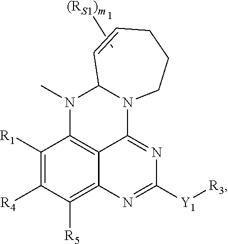

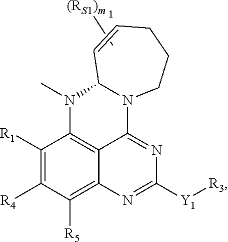

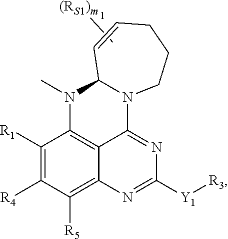

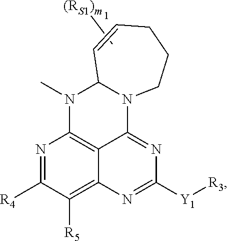

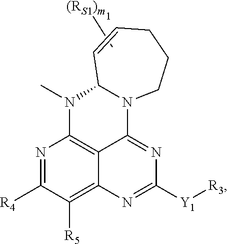

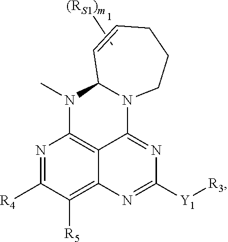

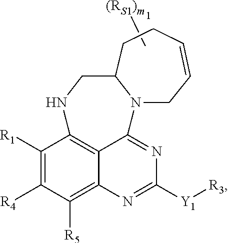

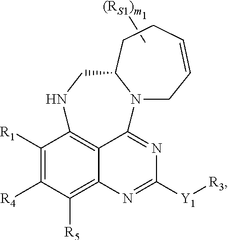

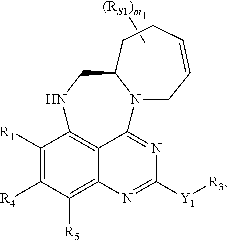

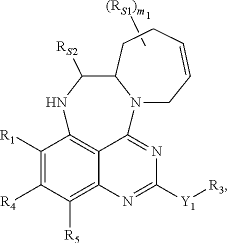

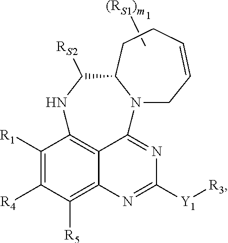

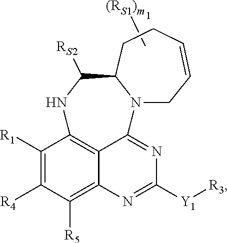

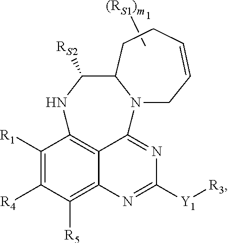

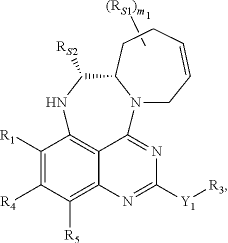

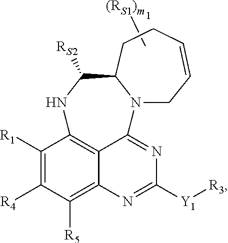

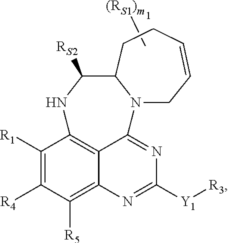

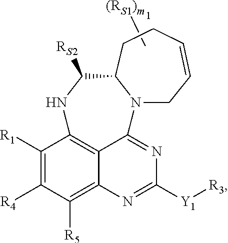

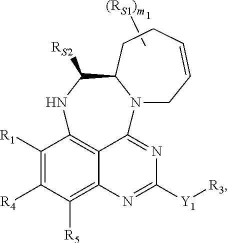

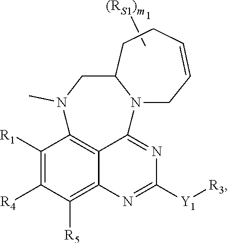

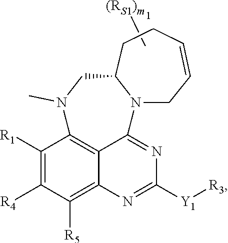

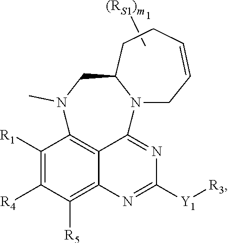

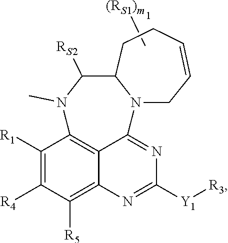

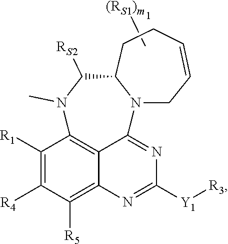

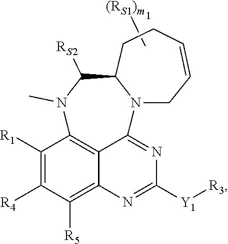

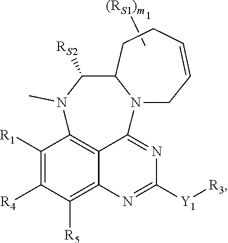

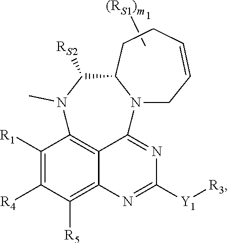

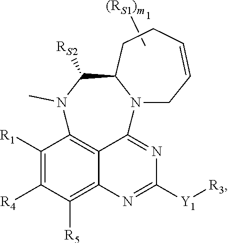

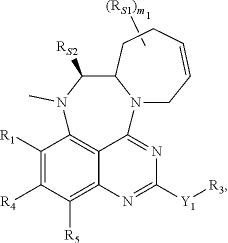

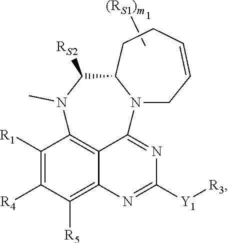

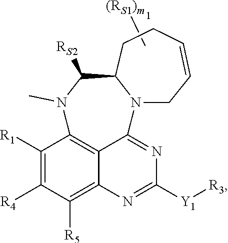

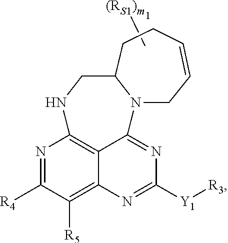

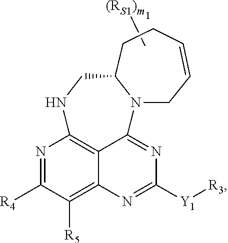

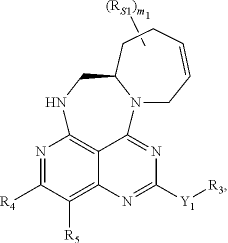

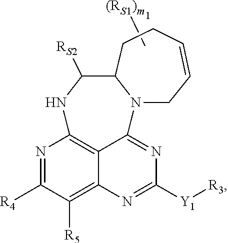

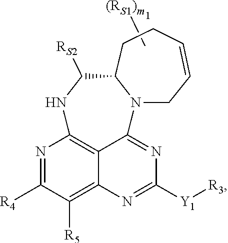

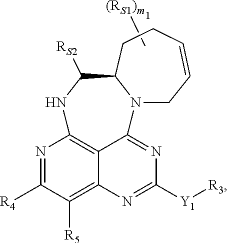

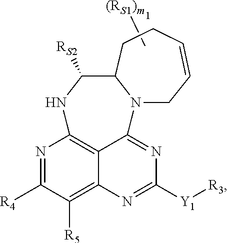

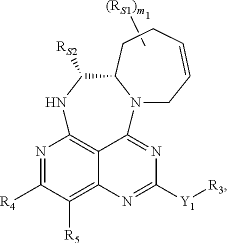

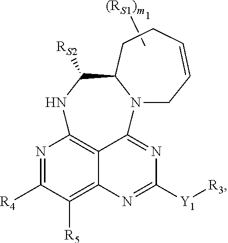

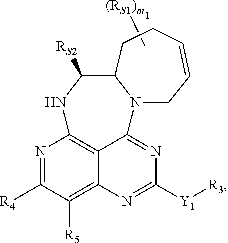

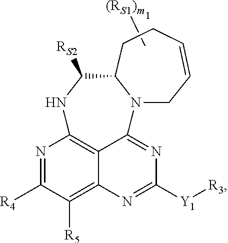

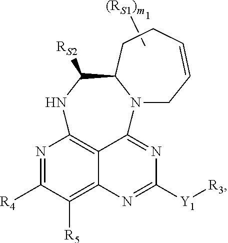

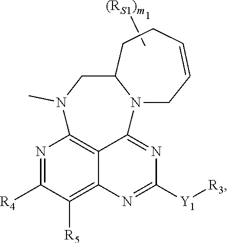

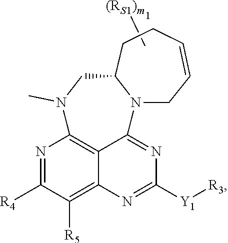

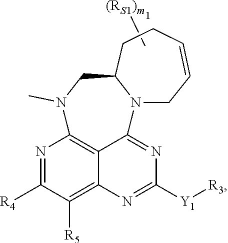

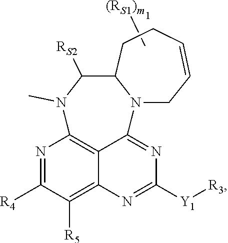

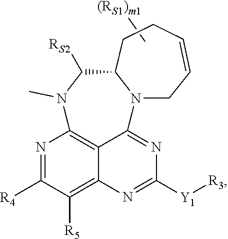

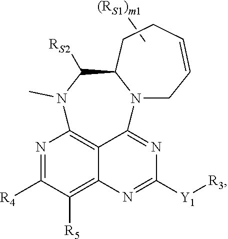

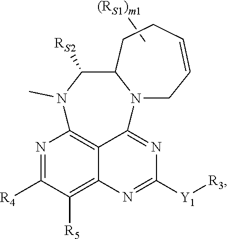

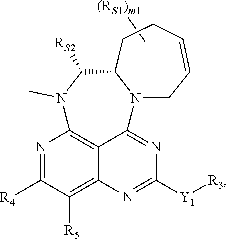

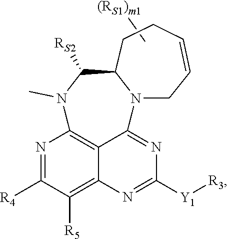

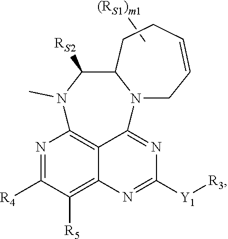

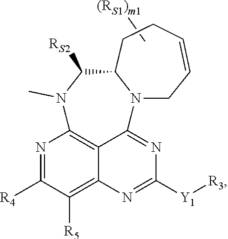

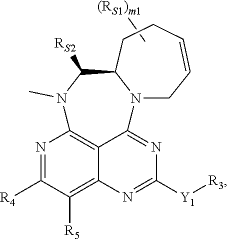

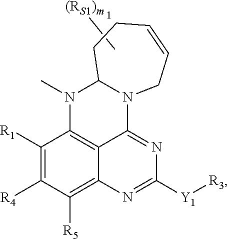

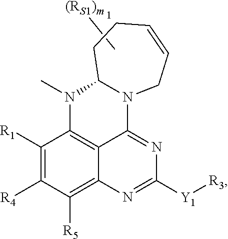

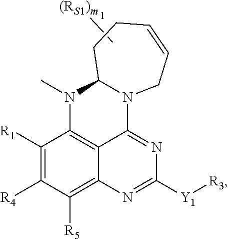

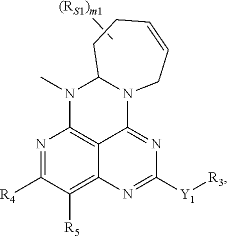

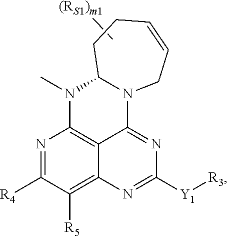

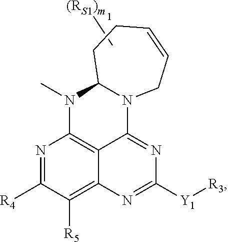

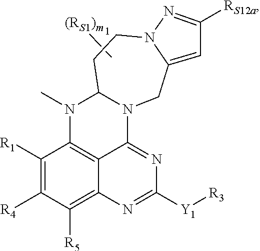

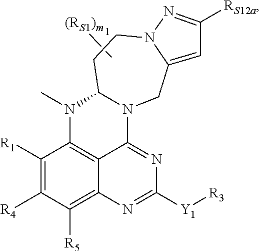

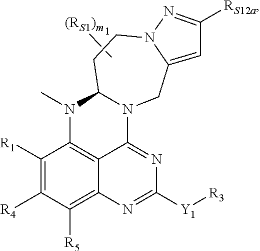

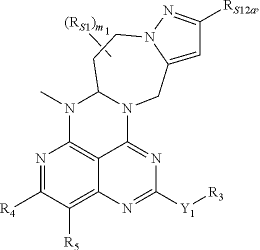

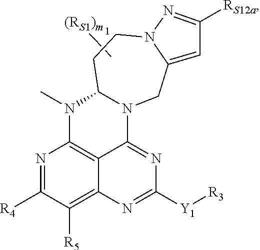

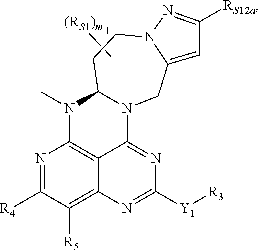

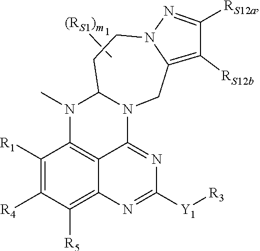

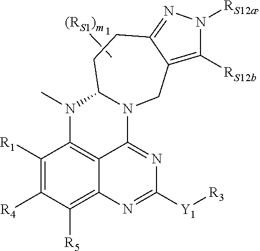

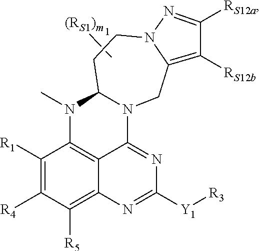

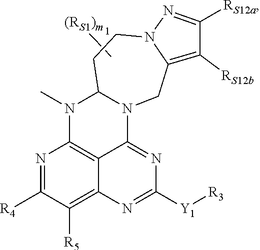

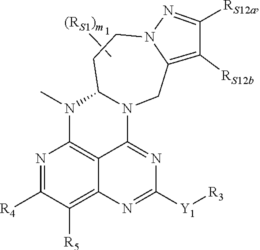

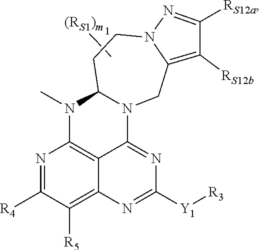

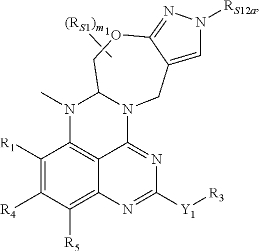

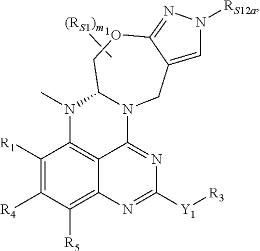

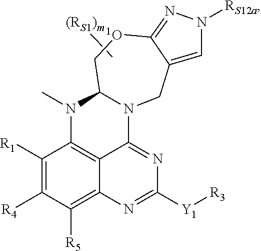

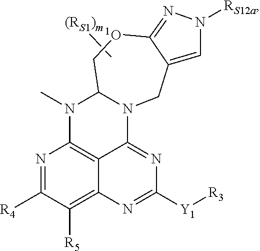

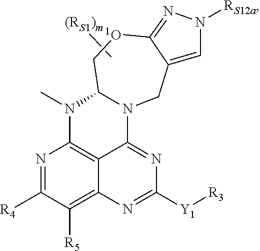

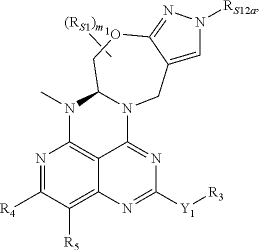

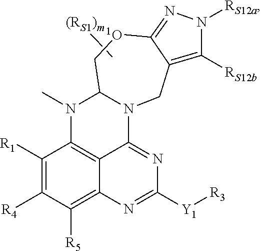

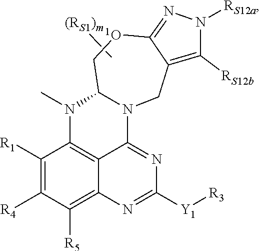

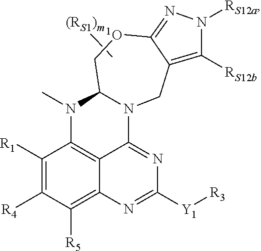

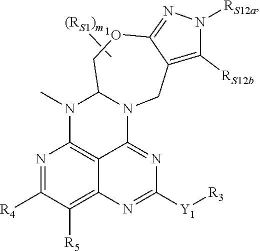

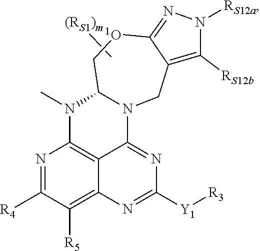

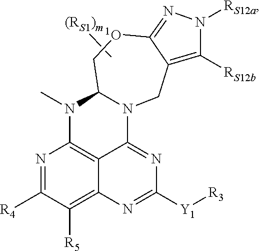

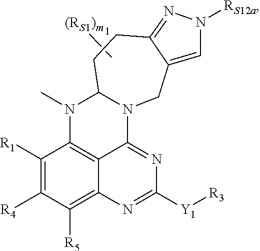

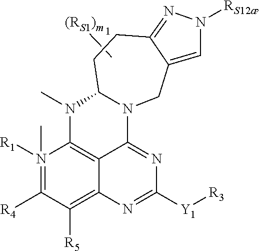

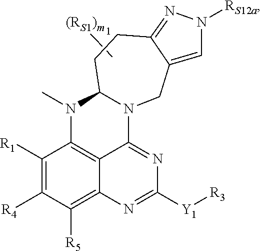

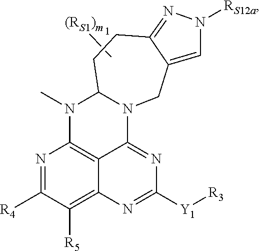

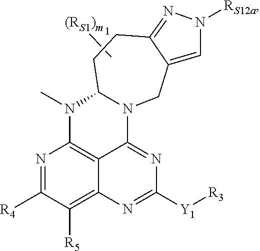

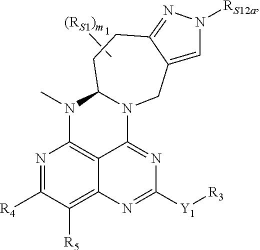

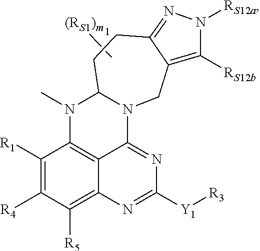

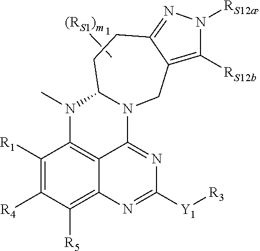

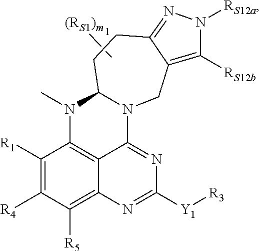

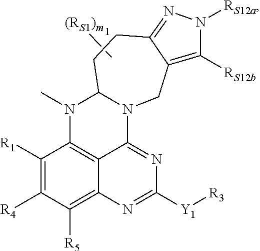

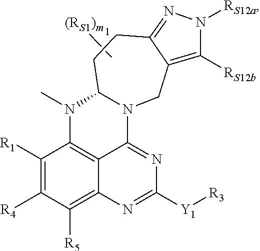

or

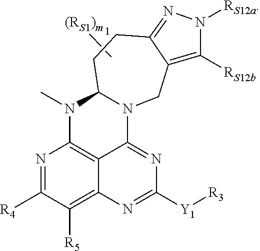

[19]. The compound of any one of [1] to [18], wherein, Y₁ is O.

[20]. The compound ofany one of [1] to [19], wherein, each of R₃₈ and R₃₉ is independently hydrogen or deuterium.

[21]. The compound ofany one of [1] to [20], wherein, n₅ is 1.

[22]. The compound of any one of [1] to [21], wherein,
Ring I is a 4-6 membered cycloalkyl ring.

[23]. The compound ofany one of [1] to [22], wherein:
Ring J is a 4-6 membered heterocyclic ring containing 1 or 2 heteroatoms selected from N, or S.

[24]. The compound of any one of [1] to [23], wherein:

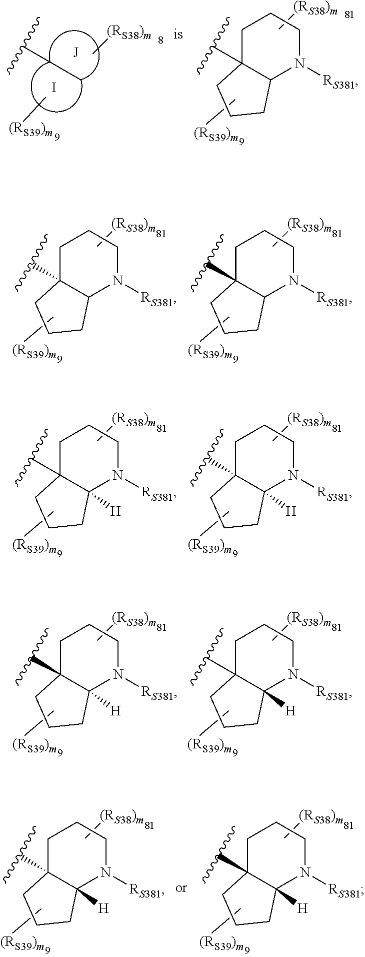

Wherein, R_{S381} is hydrogen or R_{S38};
m₈₁ is 0, 1, 2, 3, 4, 5, 6, 7, or 8.

[25]. The compound of any one of [1] to [24], wherein:
R_{S381} is hydrogen, deuterium, —C₁₋₆alkyl or 3-6 membered cycloalkyl, wherein, said —C₁₋₆alkyl or 3-6 membered cycloalkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl.

[26]. The compound of any one of [1] to [25], wherein, R_{S381} is hydrogen, deuterium, —CH₃, —CH₂CH₃ or cyclopropyl.

[27]. The compound of any one of [1] to [26], wherein, m₈₁ is 0.

[28]. The compound of any one of [1] to [27], wherein:
m₉ is 0, 1 or 2.

[29]. The compound of any one of [1] to [28], wherein:
m₉ is 0.

[30]. The compound of any one of [1] to [29], wherein:
m₉ is 1.

[31]. The compound of any one of [1] to [30], wherein:

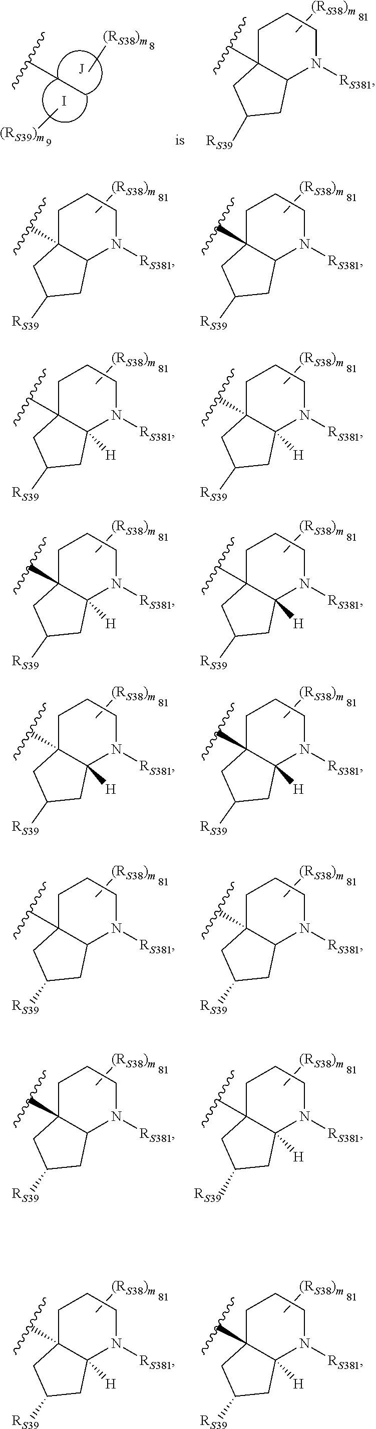

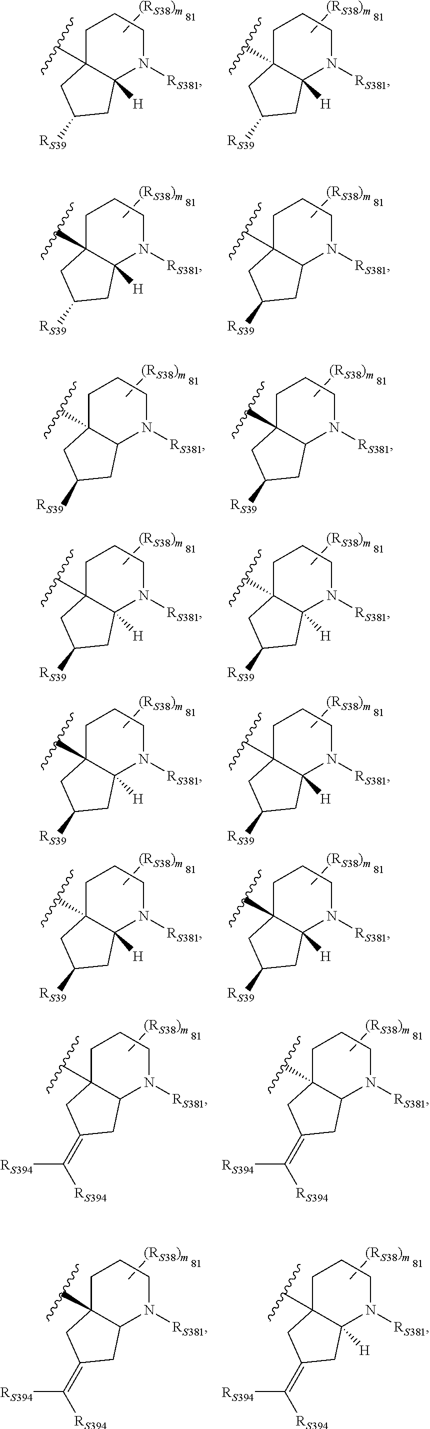

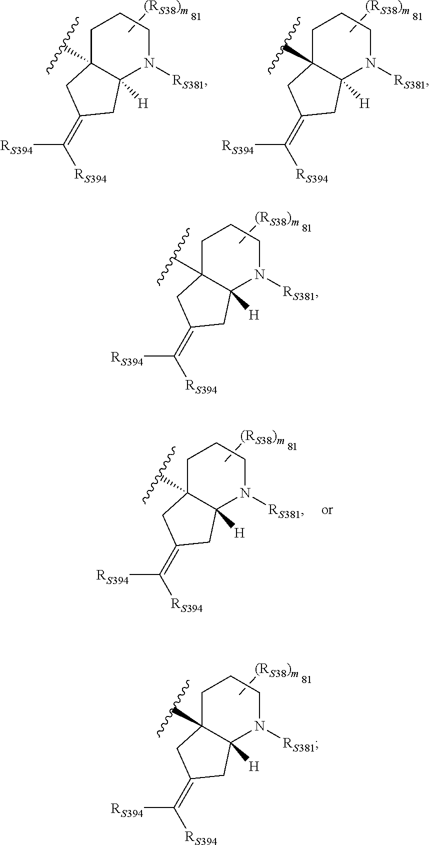

Wherein, R_{S394} is hydrogen or R_{S391}.

[32]. The compound of any one of [1] to [31], wherein:
R_{S39} is halogen;

Preferably, R_{S39} is —F.

[33]. The compound of any one of [1] to [32], wherein:
R_{S391} is hydrogen, deuterium, halogen, or —C₁₋₆alkyl; wherein, said —C₁₋₆alkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl;
Preferably, R_{S391} is hydrogen, deuterium, —F, or —CH₃.

[34]. The compound of any one of [1] to [33], wherein:

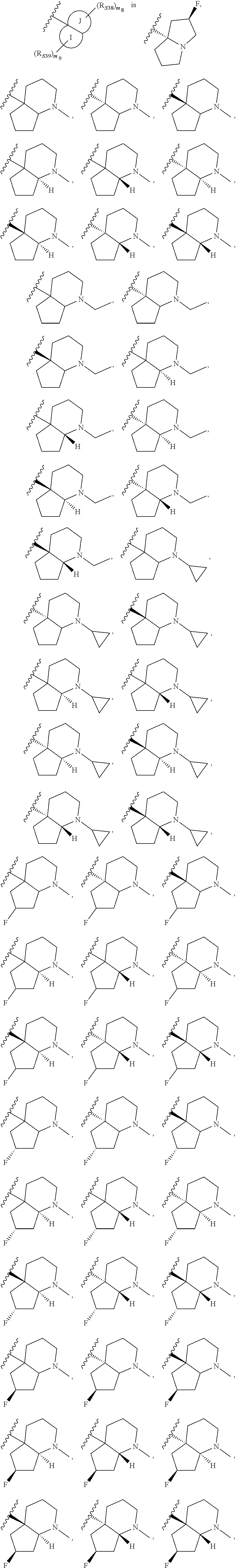

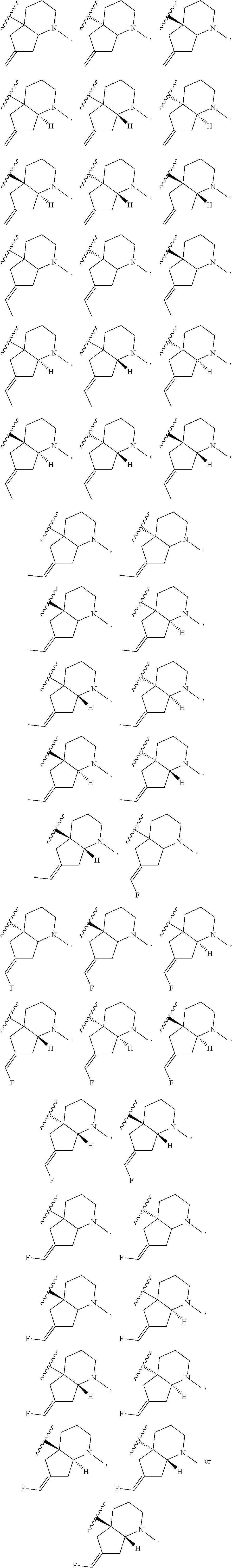

[35]. The compound of any of [1] to [34], wherein:

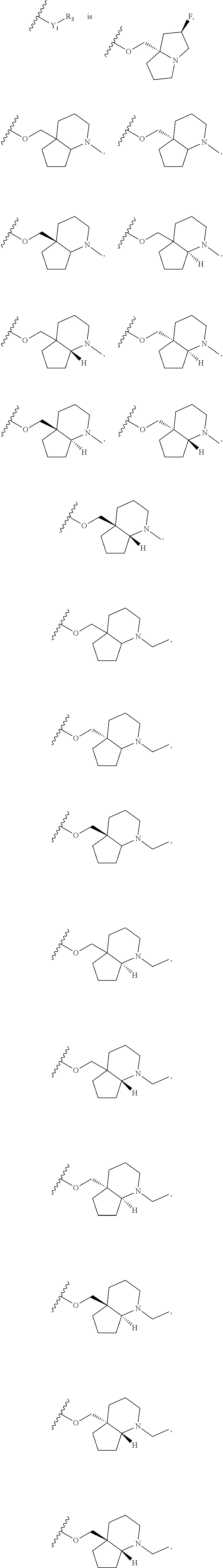

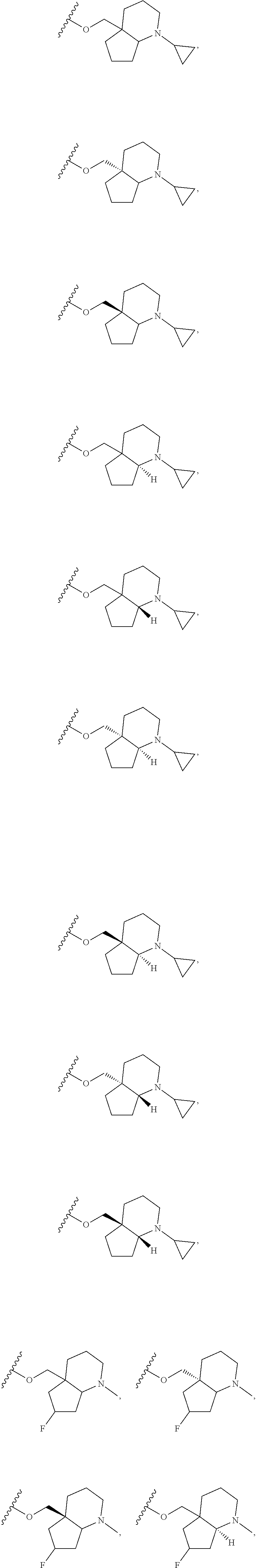

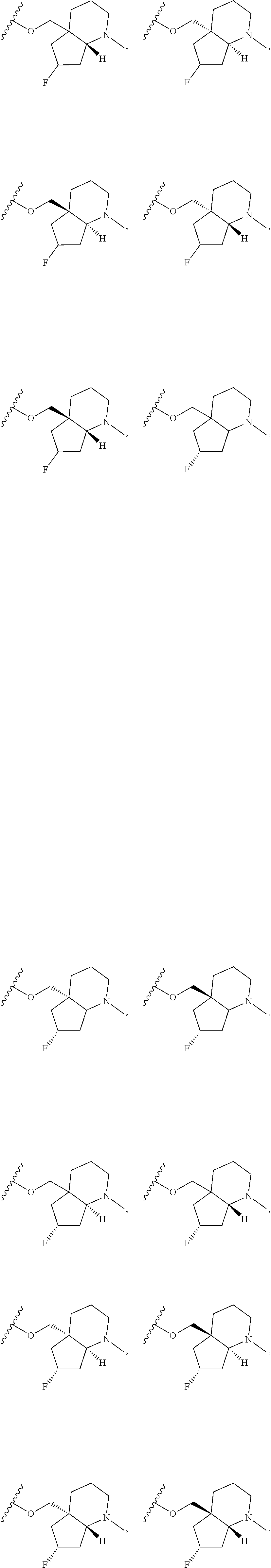

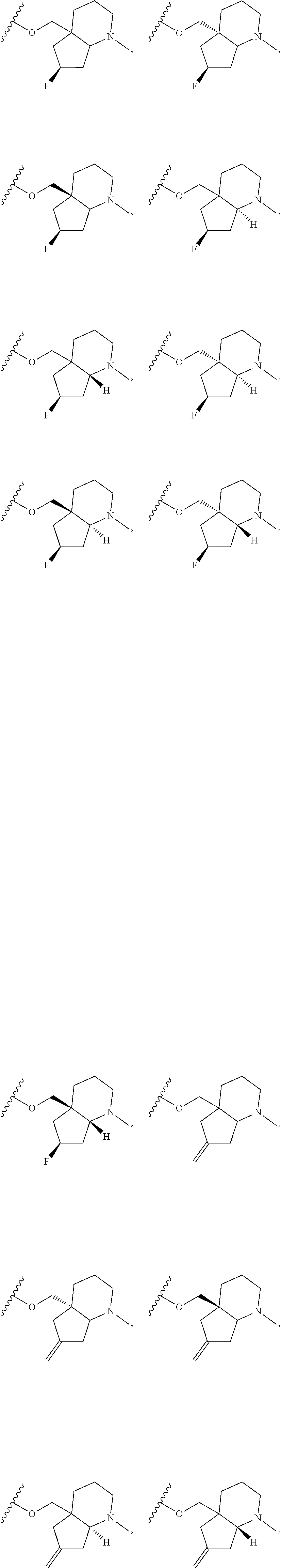

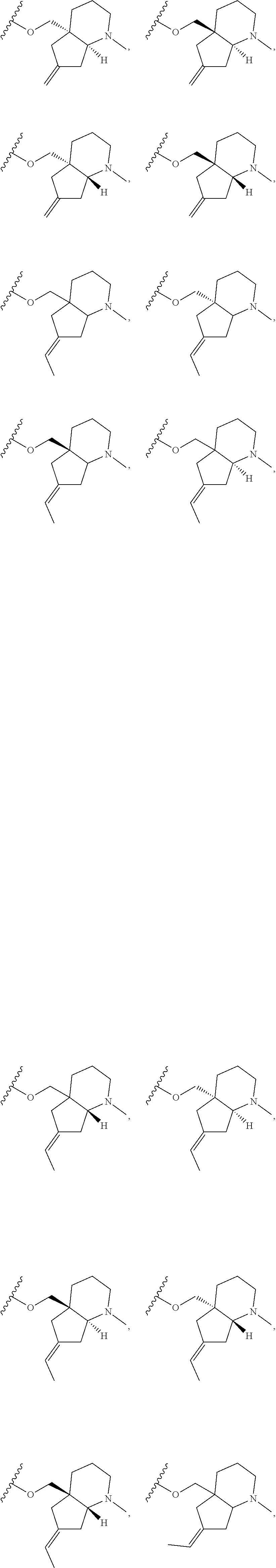

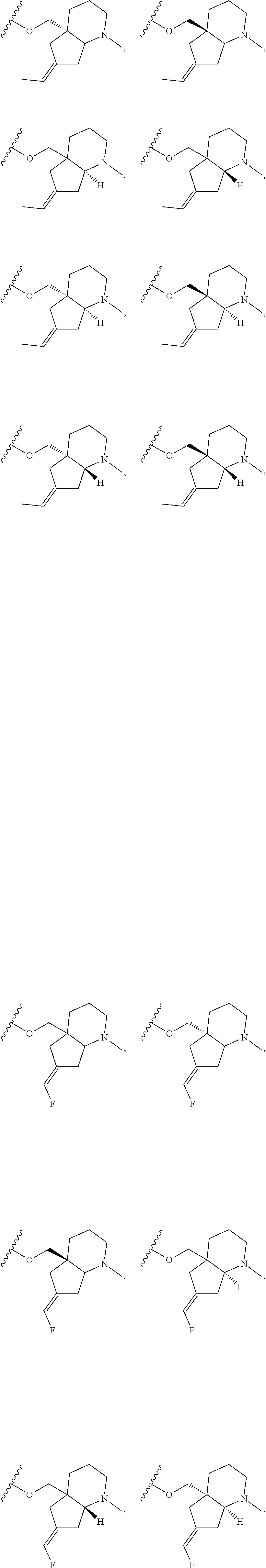

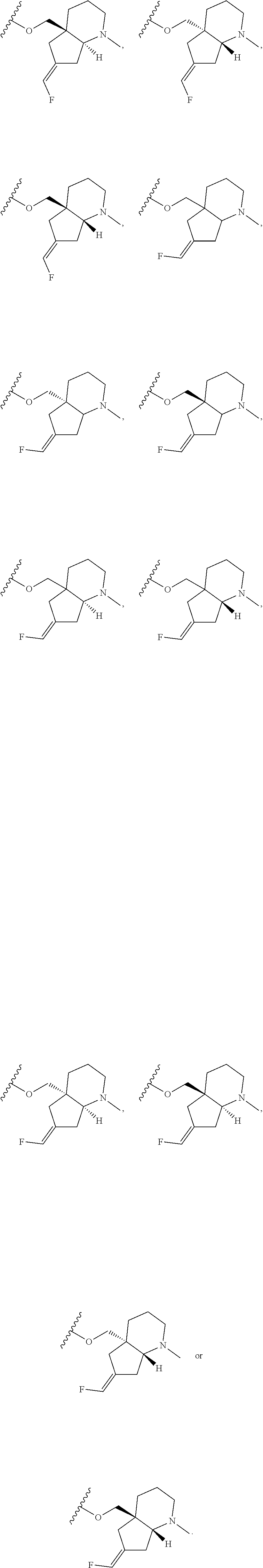

[36]. The compound of any one of [1] to [21], wherein:
Ring B is a 4-6 membered heterocyclic ring containing the fused N atom.

[37]. The compound of any one of [1] to [21] and [36], wherein:
Ring C is a 4-6 membered heterocyclic ring containing the fused N atom.

[38]. The compound of any of [1] to [21] and [36] to [37], wherein:

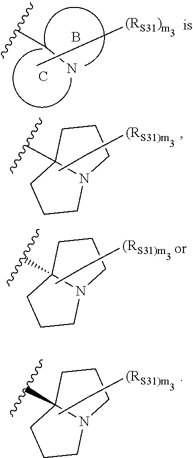

[39]. The compound of any one of [1] to [21] and [36] to [38], wherein, m₃ is 0, 1, 2, 3, or 4.

[40]. The compound of any one of [1] to [21] and [31] to [39], wherein, R_{S31} is deuterium or —F;
Optionally, two R_{S31} together the carbon atom to which they are both attached form

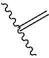

or cyclopropyl; wherein, said

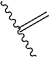

or cyclopropyl is independently unsubstituted or substituted with 1, 2 or 3 R_{S311}; or
Optionally, two adjacent R_{S31} together with the carbon atoms to which they are respectively attached form a 5-10 membered heterocyclic ring containing 1 or 2 heteroatoms selected from N or O, a phenyl ring or a 5-10 membered heteroaryl ring containing 1 or 2 heteroatoms selected from N, O or S, wherein, each of rings is independently unsubstituted or substituted with 1, 2 or 3 R_{S312}.

[41]. The compound of any one of [1] to [21] and [36] to [40], wherein:

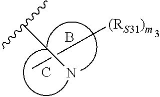

is selected from

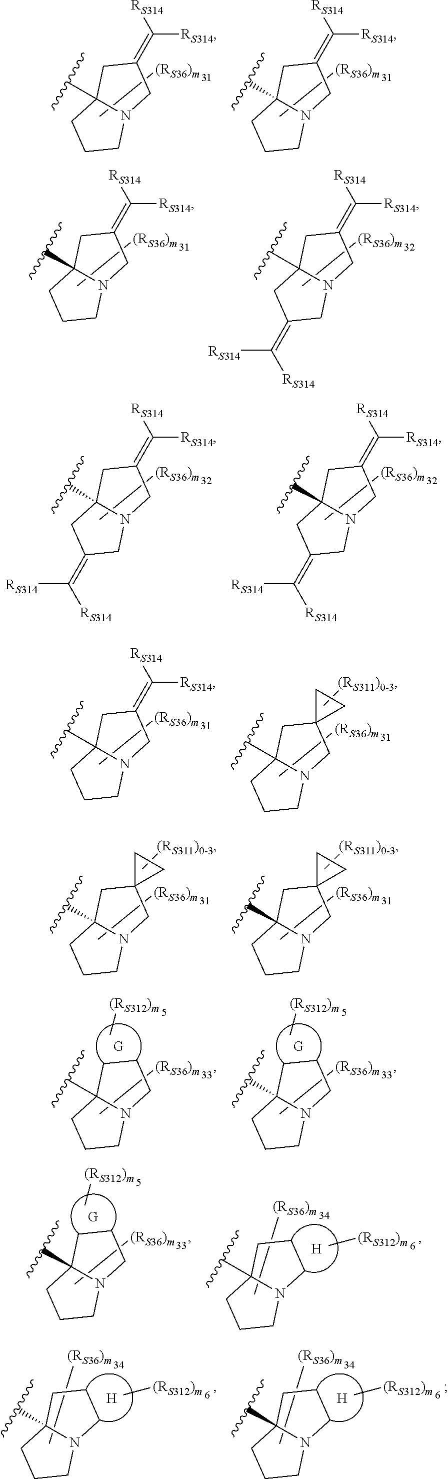

Wherein,
Ring G is a 5-6 membered heterocyclic ring containing 1 or 2 heteroatoms selected from N or O, a phenyl ring, a 5-6 membered heteroaryl ring containing 1, 2 or 3 heteroatoms selected from N, O, or S;
Ring H is a 5-10 membered heterocyclic ring containing 1 or 2 heteroatoms selected from N or O, a phenyl ring, a 5-10 membered heteroaryl ring containing 1 or 2 heteroatoms selected from N, O, or S;
The definition of R_{S36} is same as R_{S31};
R_{S314} is hydrogen or R_{S311};
m₃₁ is 0, 1, 2, 3, 4, 5, 6, 7, 8, 9 or 10;
m₃₂ is 0, 1, 2, 3, 4, 5, 6, 7 or 8;
m₃₃ is 0, 1, 2, 3, 4, 5, 6, 7, 8, 9, or 10;
m₃₄ is 0, 1, 2, 3, 4, 5, 6, 7, 8, 9, 10, 11 or 12;
m₅ is 0, 1, 2, 3, 4, 5, or 6;
m₆ is 0, 1, 2, 3, 4, 5, or 6.

[42]. The compound of any one of [1] to [21] and [36] to [41], wherein:

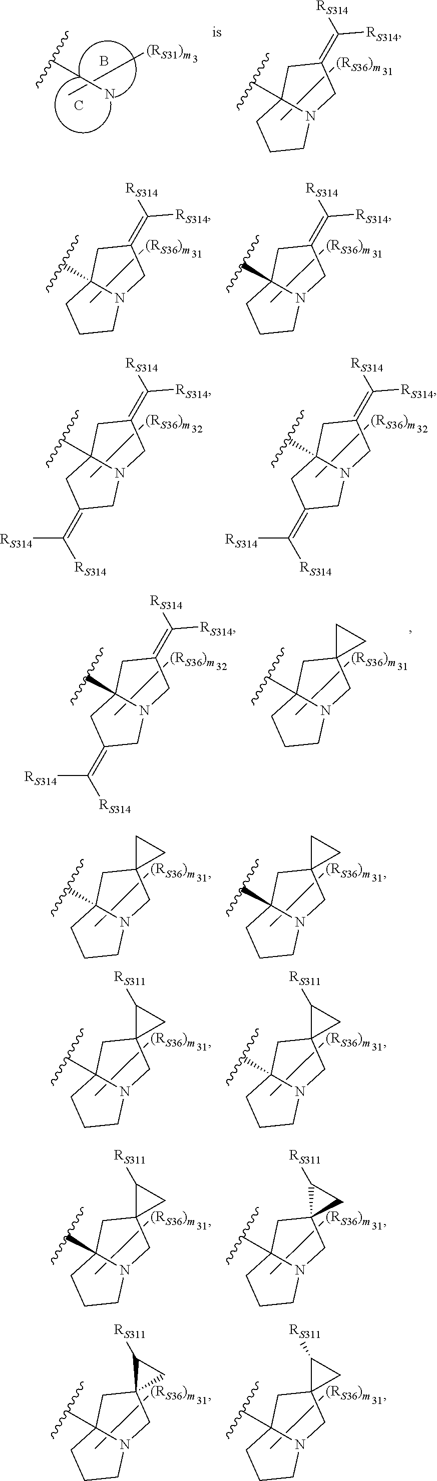

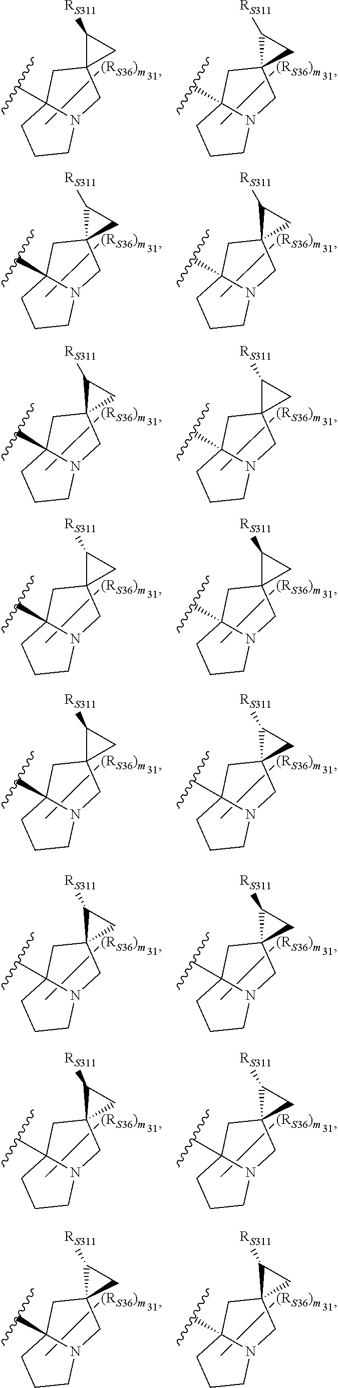

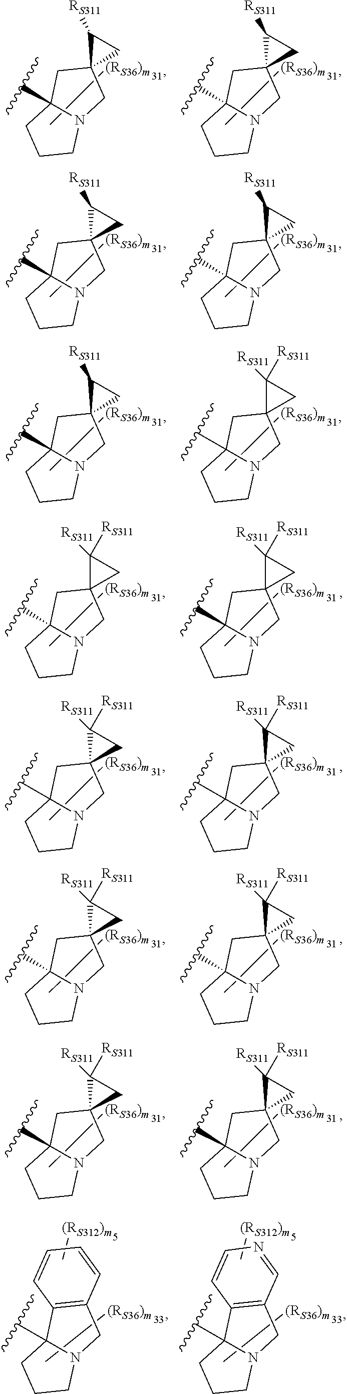

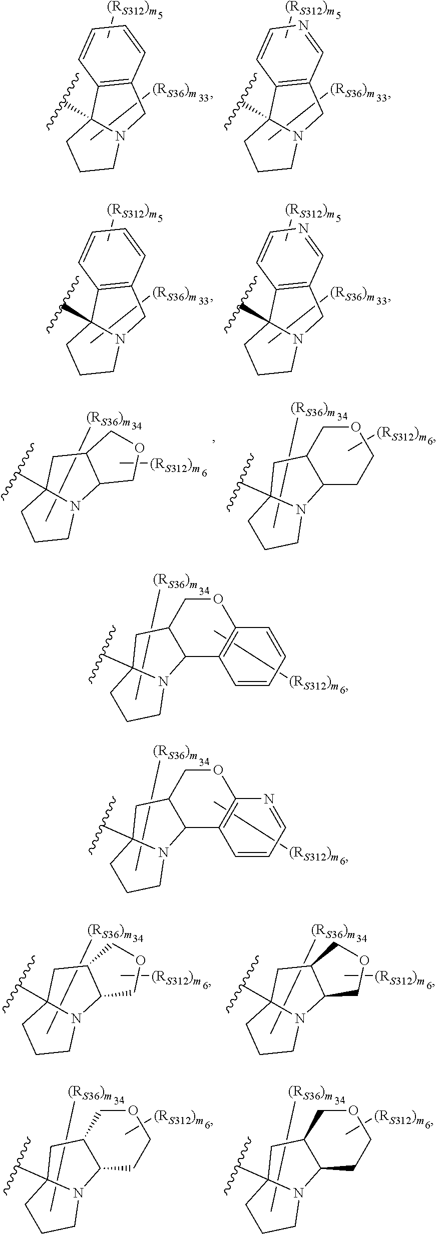

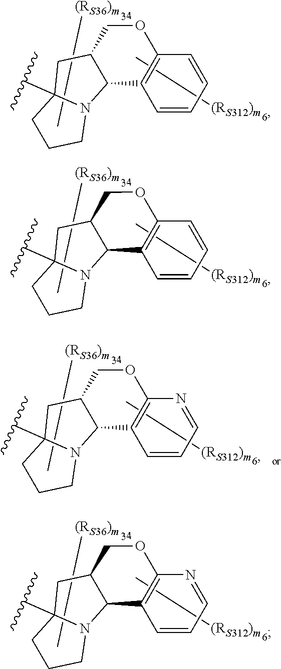

Wherein,
m₃₁ is 0, 1, 2, 3, 4, 5, 6, 7, 8, 9 or 10;
m₃₂ is 0, 1, 2, 3, 4, 5, 6, 7 or 8;
m₃₃ is 0, 1, 2, 3, 4, 5, 6, 7, 8, 9, or 10;
m₃₄ is 0, 1, 2, 3, 4, 5, 6, 7, 8, 9, 10, 11 or 12;
m₅ is 0, 1, 2, 3, 4, 5, or 6;
m₆ is 0, 1, 2, 3, 4, 5, or 6.

[43]. The compound of any one of [1] to [21] and [36] to [42], wherein:
R_{S311} is independently deuterium, halogen, —C₁₋₆alkyl, haloC₁₋₆alkyl, haloC₁₋₆alkoxy, —OH, —OC₁₋₆alkyl, —CN, —NH₂, —NH(C₁₋₆alkyl) or —N(C₁₋₆alkyl)₂; wherein, said —C₁₋₆alkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆-alkyl)₂, —OH or —OC₁₋₆alkyl;
R_{S312} is independently deuterium, halogen, —C₁₋₆alkyl, haloC₁₋₆alkyl, haloC₁₋₆alkoxy, —OH, —OC₁₋₆alkyl, —CN, —NH₂, —NH(C₁₋₆alkyl) or —N(C₁₋₆alkyl)₂; wherein, said —C₁₋₆alkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl;
R_{S314} is independently hydrogen, deuterium, halogen, —C₁₋₆alkyl, wherein, said —C₁₋₆alkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl.

[44]. The compound of any one of [1] to [21] and [36] to [43], wherein:
R_{S311} is independently deuterium, —F or —OCH₃;
R_{S312} is independently deuterium, —F, —OCH₃, or —CH₂OCH₃;
R_{S314} is independently hydrogen, deuterium, —F, —CH₃, —CH₂OCH₃, —CH₂CH₂CH₃, —CH(CH₃)₂, —CHF₂, —CH₂CH(CH₃)₂.

[45]. The compound of any one of [1] to [21] and [36] to [44], wherein:
R_{S36} is deuterium or —F.

[46]. The compound of any one of [1] to [21] and [36] to [45], wherein:
m₃₁ is 0 or 1;
m₃₂ is 0 or 1;
m₃₃ is 0 or 1;
m₃₄ is 0 or 1;
m₅ is 0 or 1;
m₆ is 0 or 1.

[47]. The compound of any one of [1] to [21] and [36] to [46], wherein:

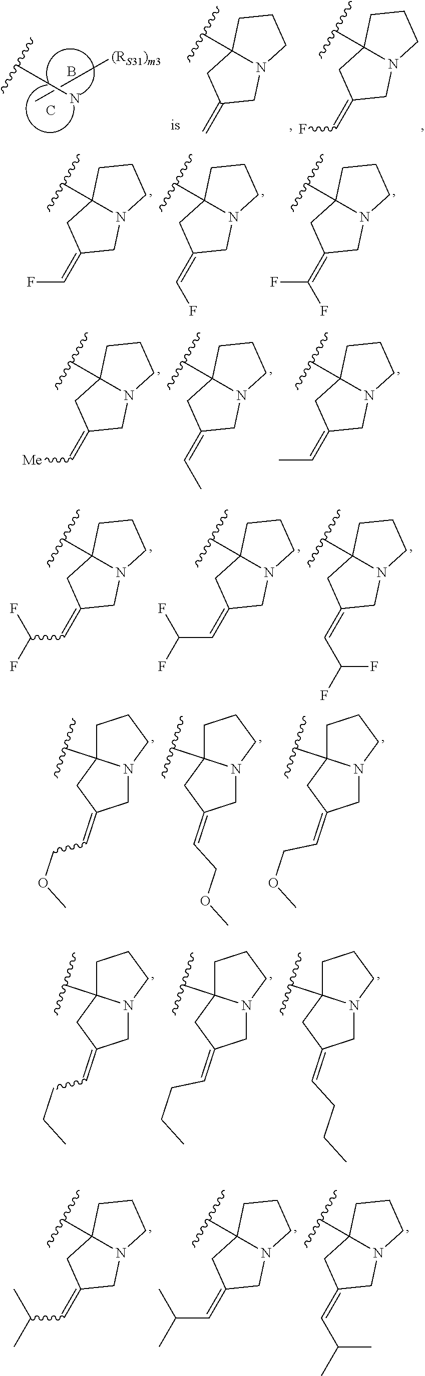

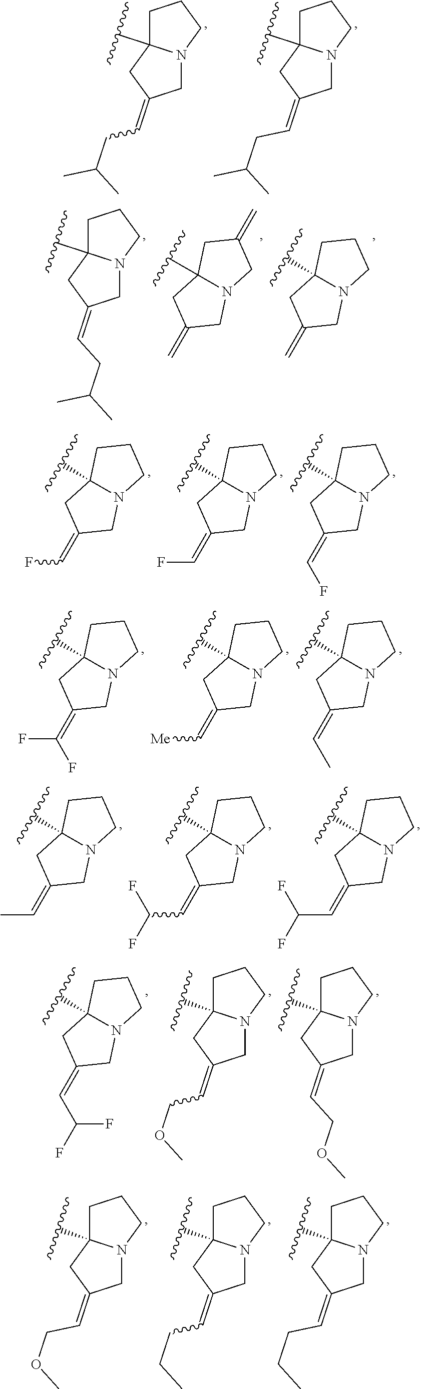

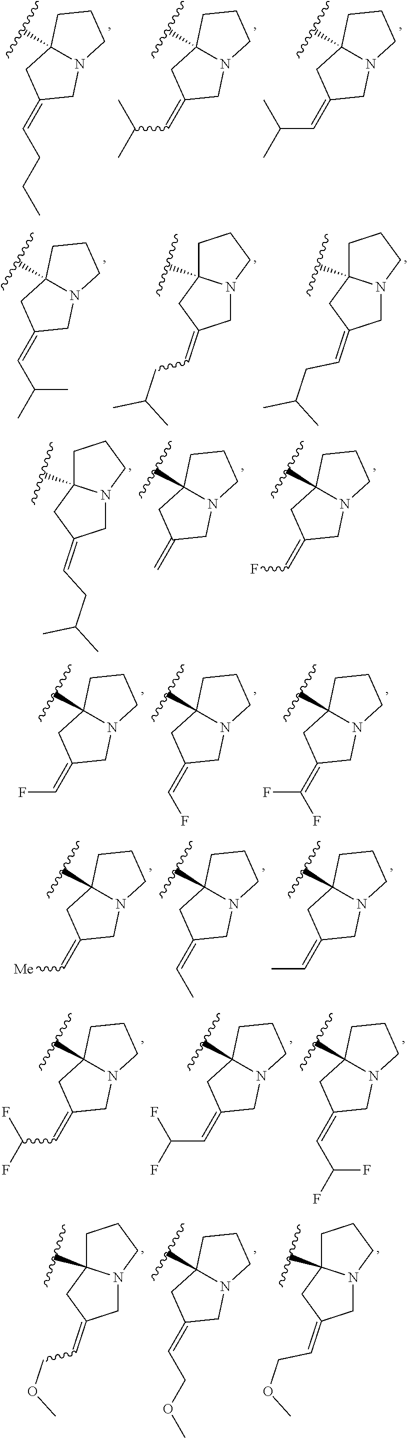

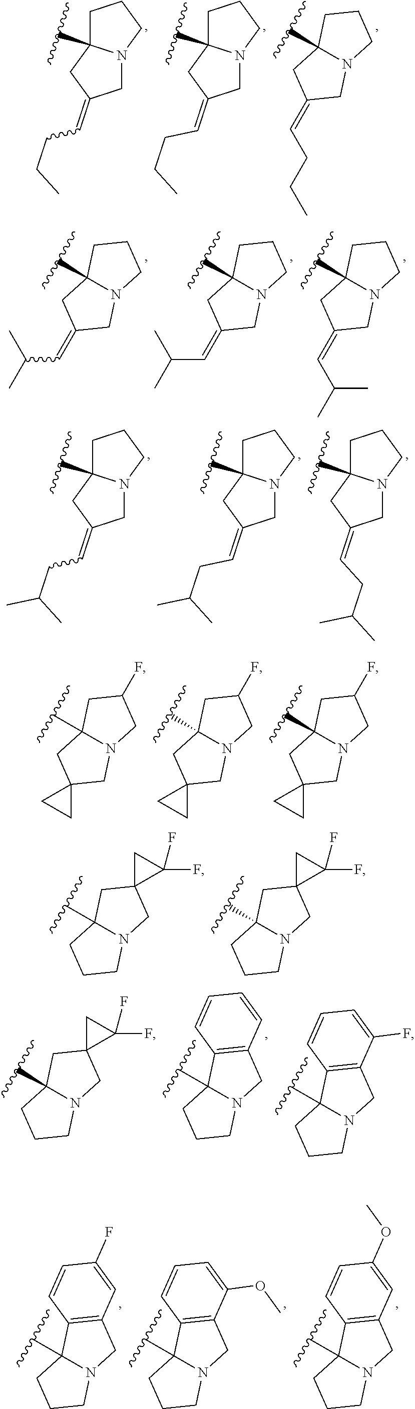

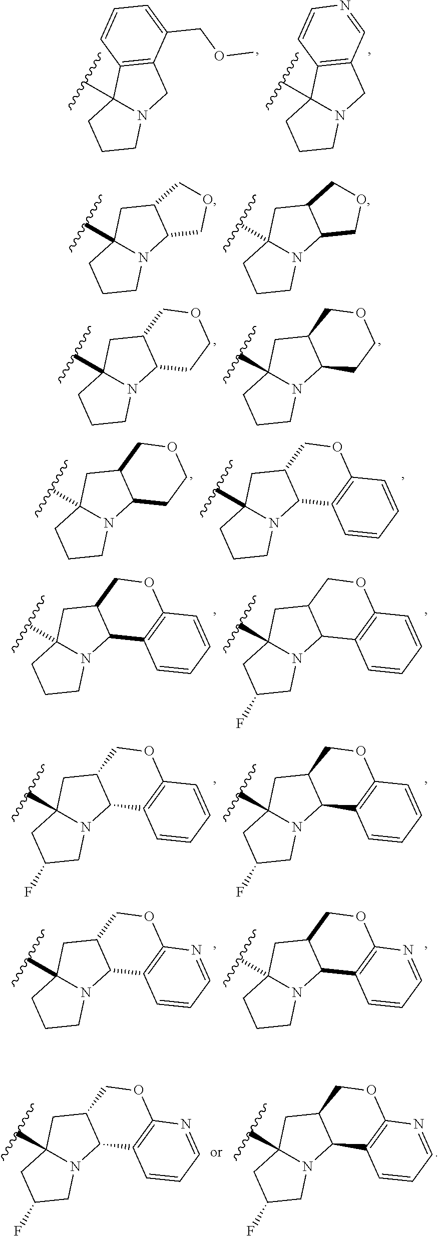

[48]. The compound of any of [1] to [21] and [36] to [47], wherein:

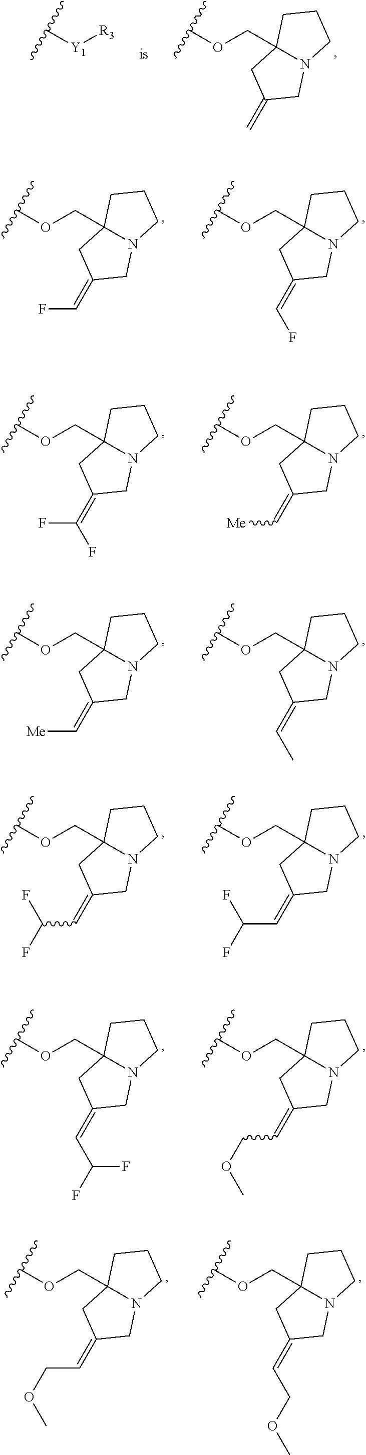

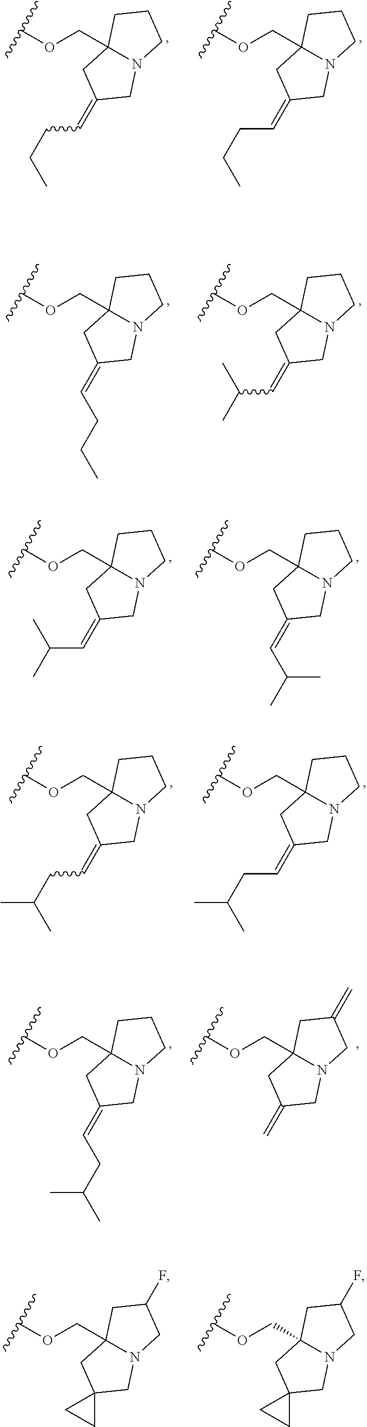

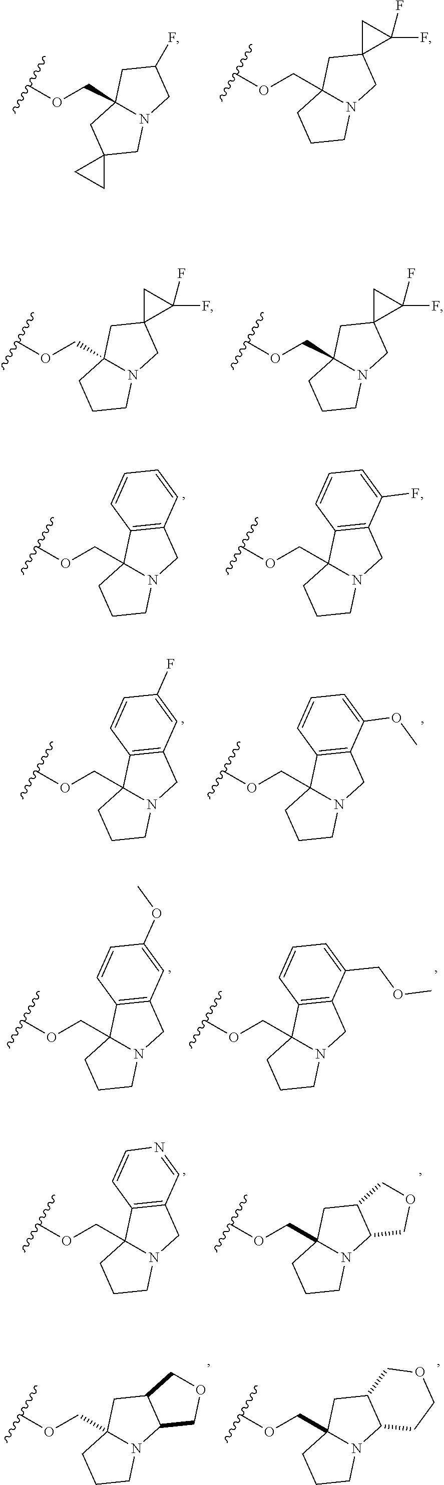

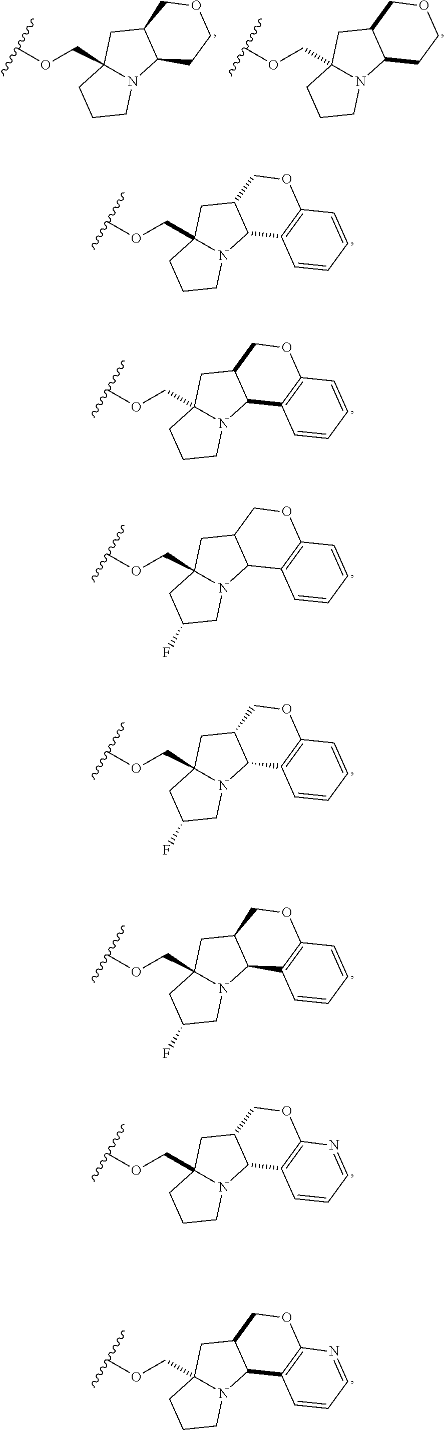

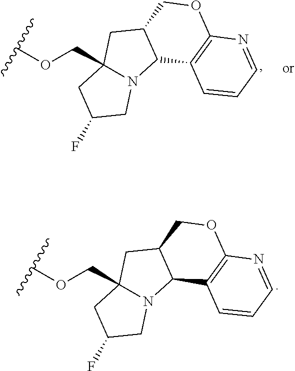

[49]. The compound of any one of [1] to [21], wherein, each of R₃₁₀ and R₃₁₁ is independently hydrogen or deuterium.

[50]. The compound of any one of [1] to [21], or [49], wherein, n₆ is 1 or 2.

[51]. The compound of any one of [1] to [21], [49] or [50], wherein:
Ring K is a 4-10 membered heterocyclic ring containing 1 or 2 heteroatoms selected from N or O.

[52]. The compound of any one of [1] to [21], [49] to [51], wherein:

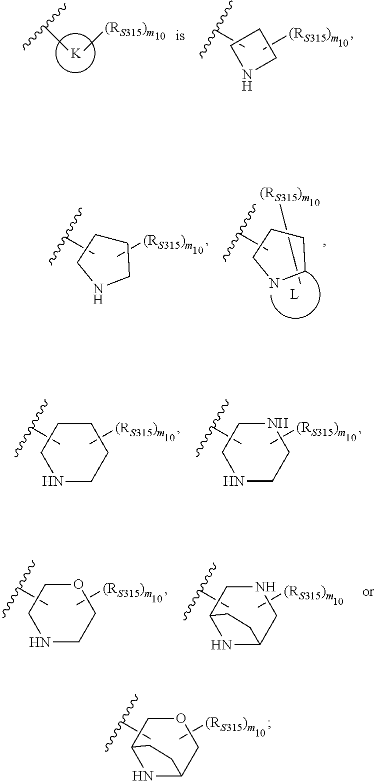

Wherein, ring L is a 4-6 membered heterocyclic ring optionally further containing 1 or 2 heteroatoms selected from N or O.

[53]. The compound of any one of [1] to [21], [49] to [52], wherein:

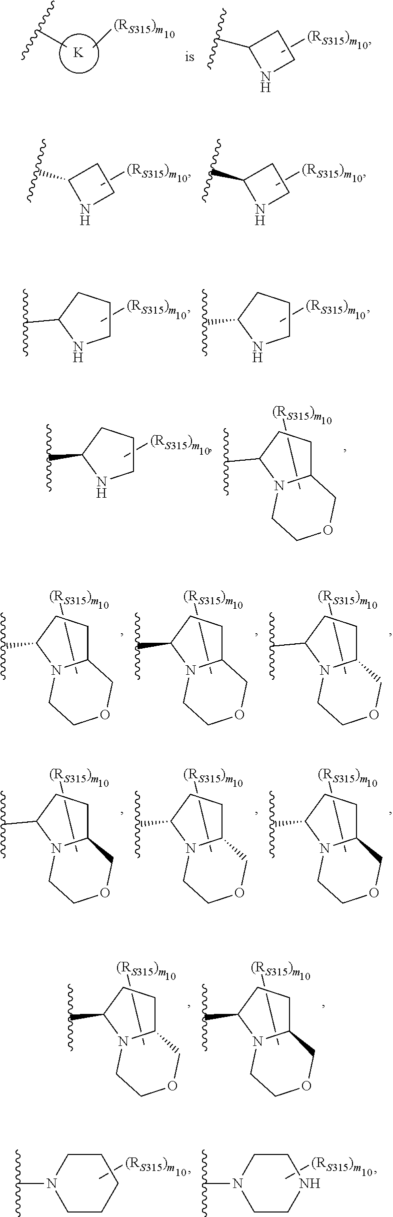

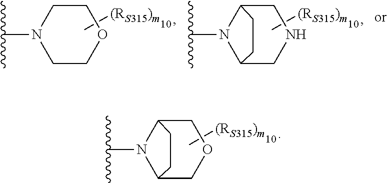

[54]. The compound of any one of [1] to [21], [49] to [53], wherein:
R_{S315} is independently deuterium, halogen, —C₁₋₆alkyl, haloC₁₋₆alkyl, haloC₁₋₆alkoxy, —OH, —OC₁₋₆alkyl, —CN, —NH₂, —NH(C₁₋₆alkyl) or —N(C₁₋₆alkyl)₂; wherein, said —C₁₋₆alkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkyl.

[55]. The compound of any one of [1] to [21], [49] to [54], wherein, R_{S315} is independently —F or —CH₃.

[56]. The compound of any one of [1] to [21], [49] to [55], wherein, m₁₀ is 0, 1, 2, or 3.

[57]. The compound of any one of [1] to [21], [49] to [56], wherein:

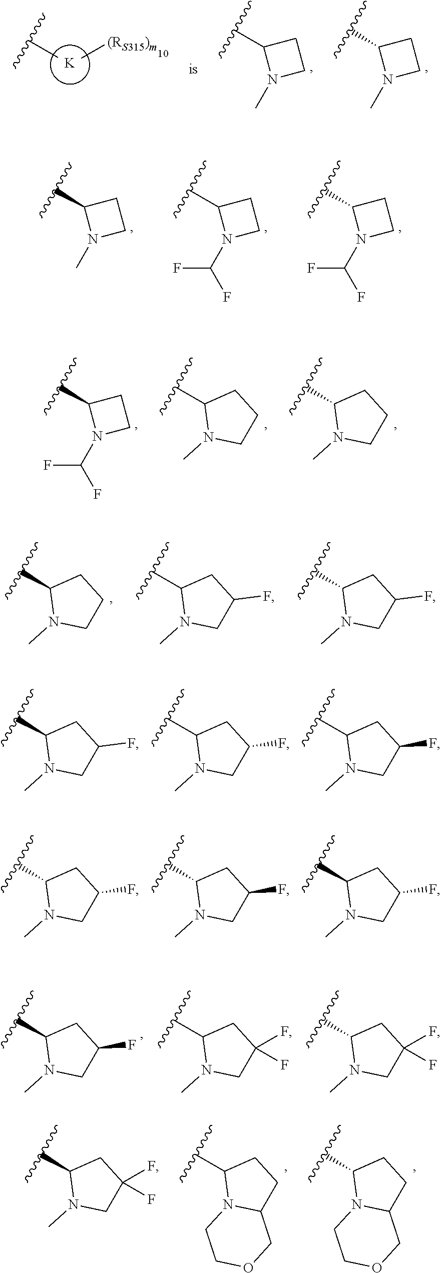

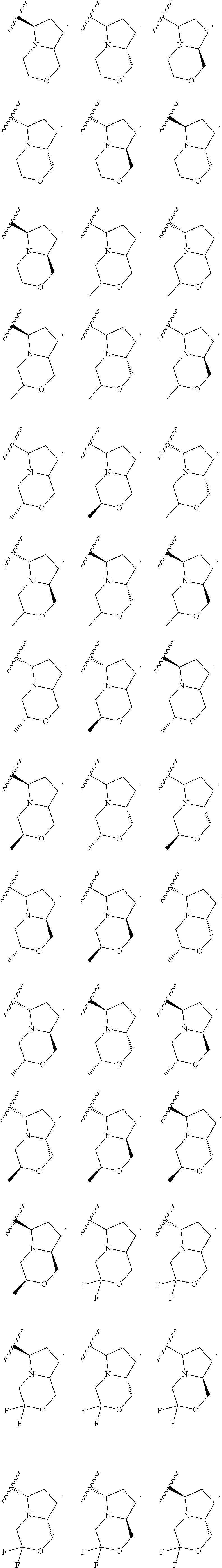

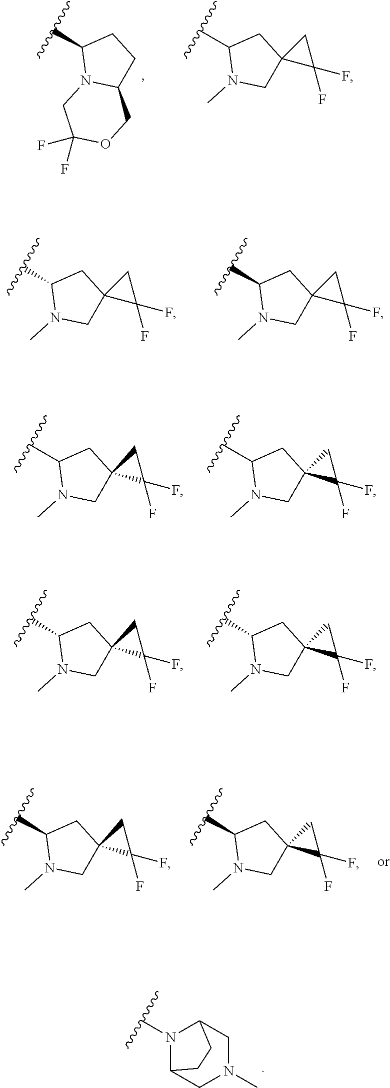

[58]. The compound of any one of [1] to [21], [49] to [57], wherein:

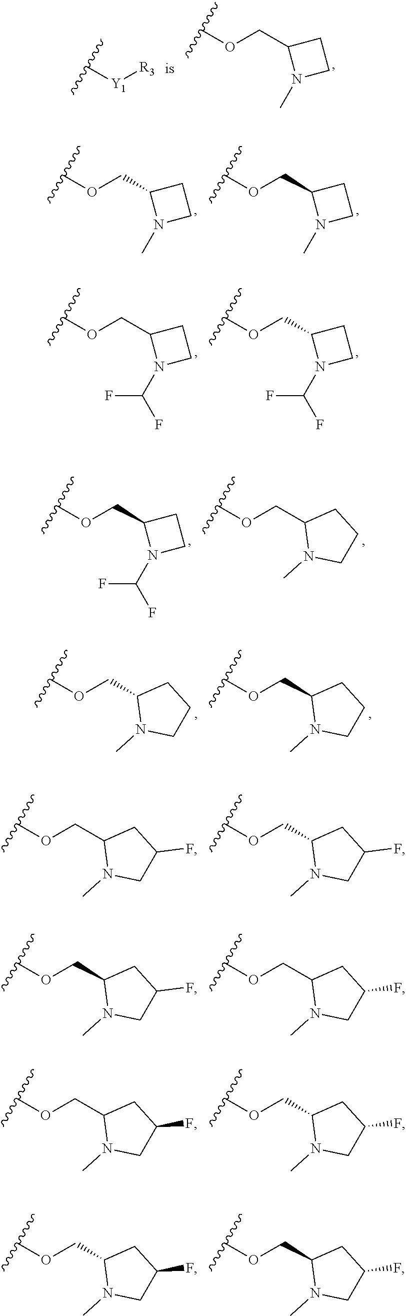

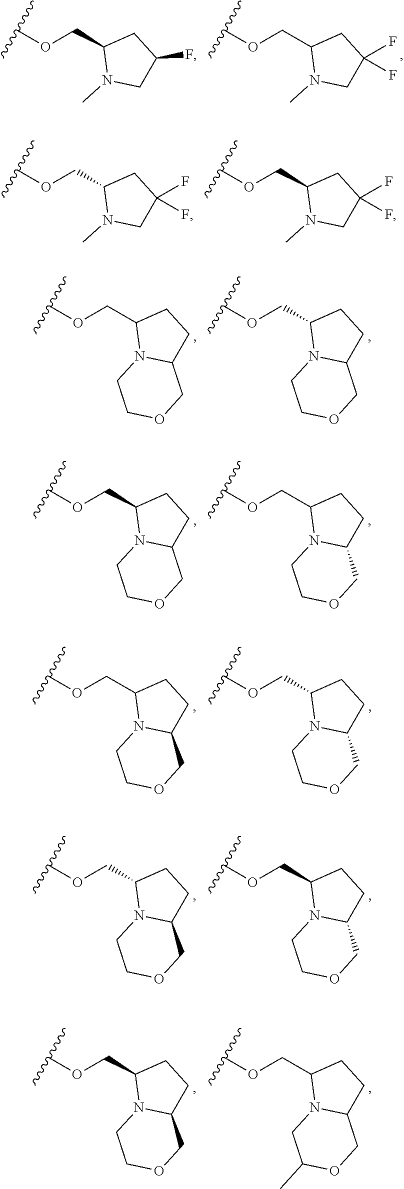

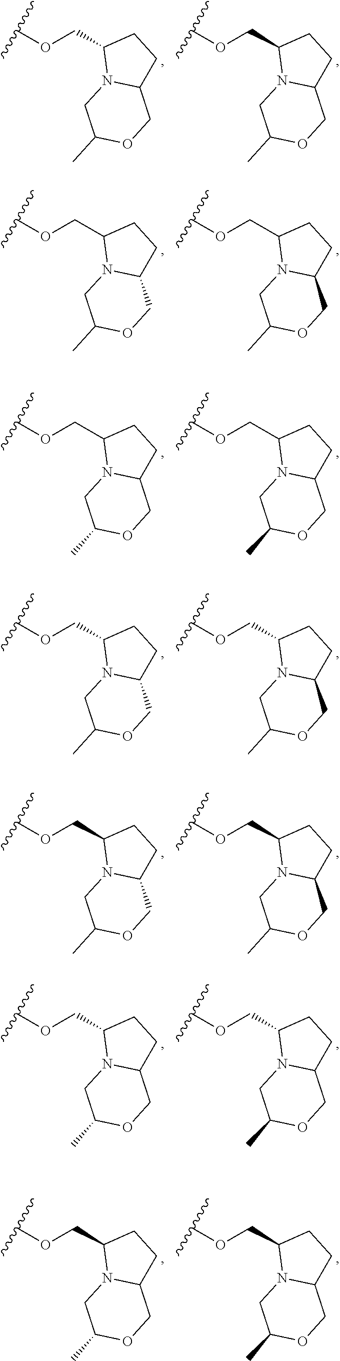

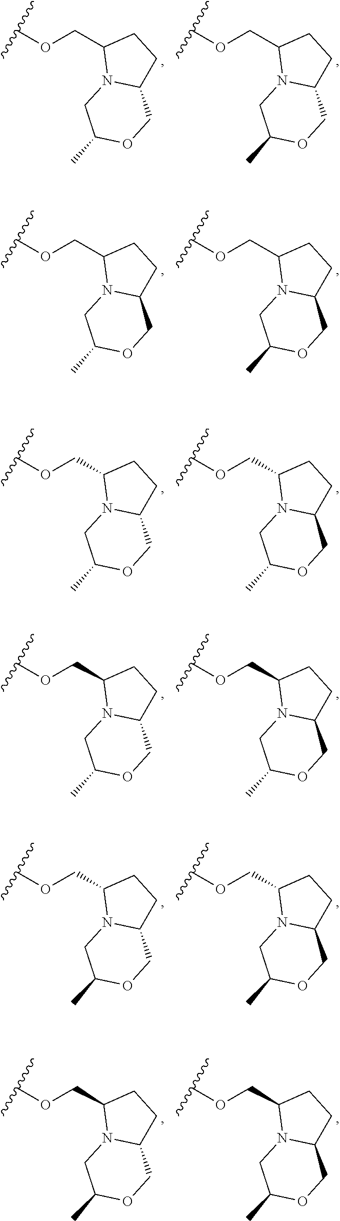

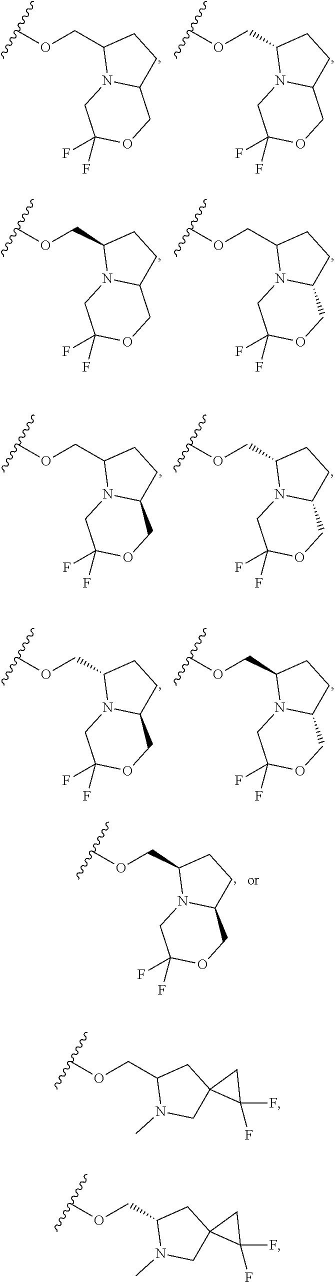

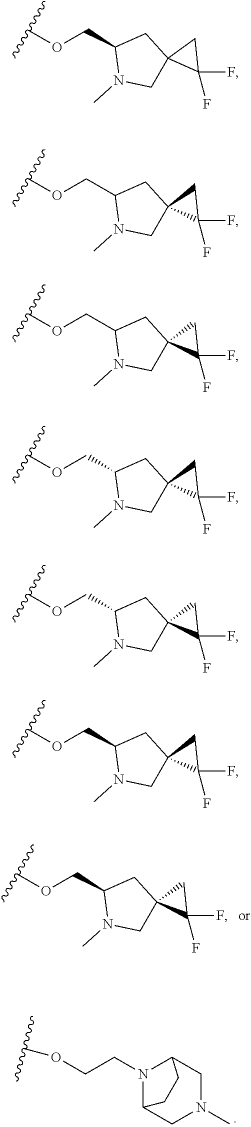

[59]. The compound of any of [1] to [21], wherein,

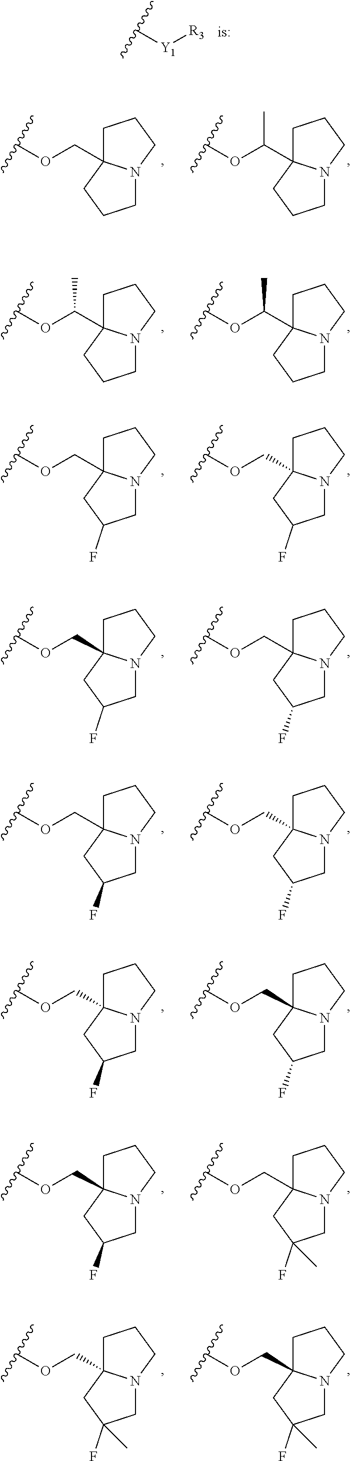

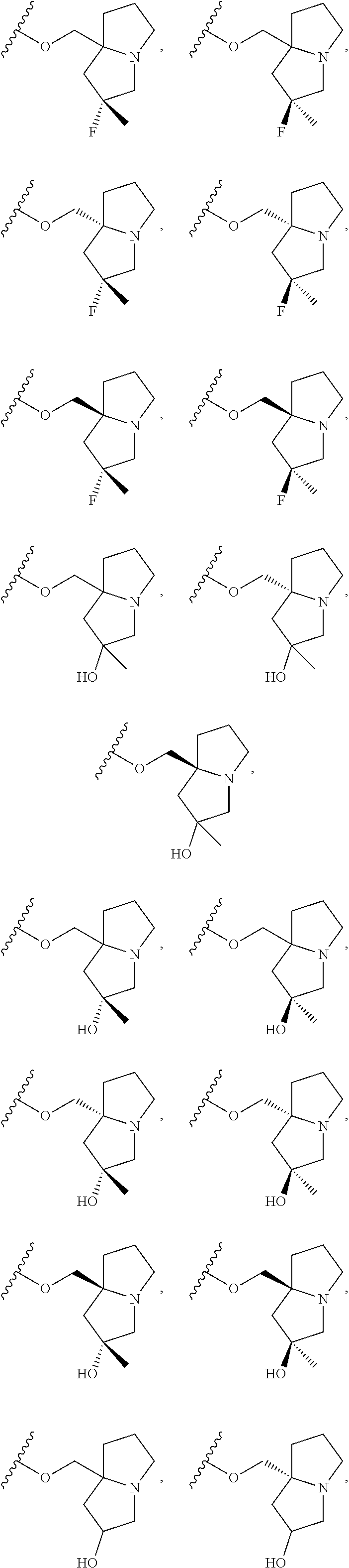

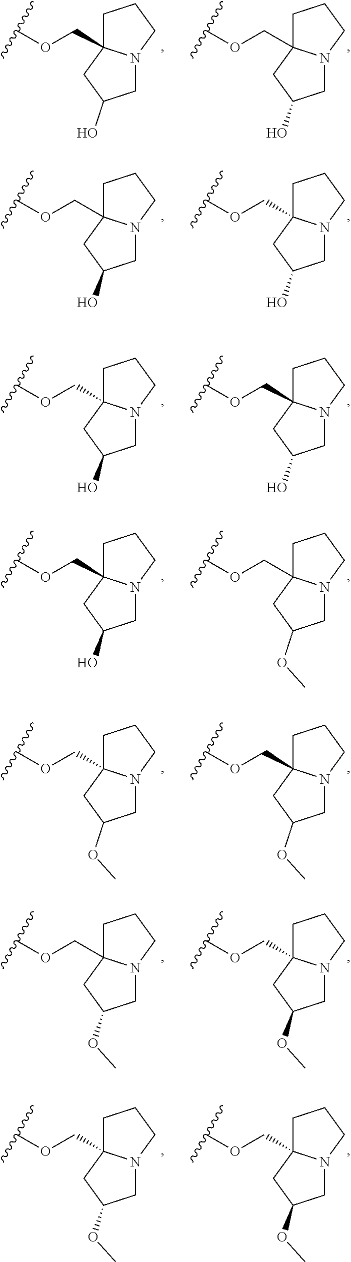

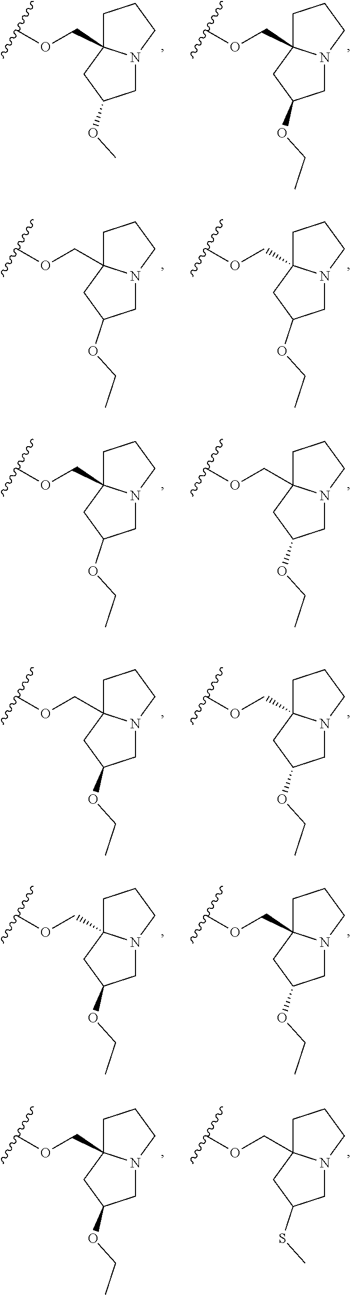

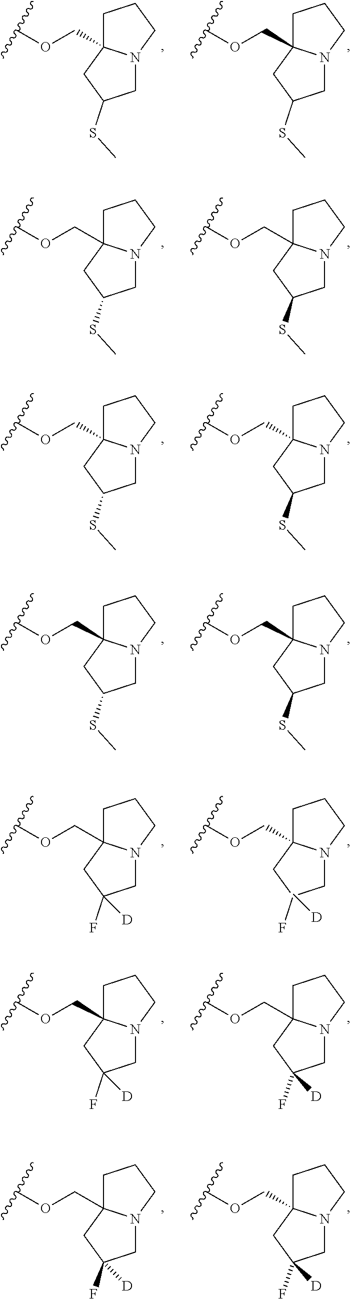

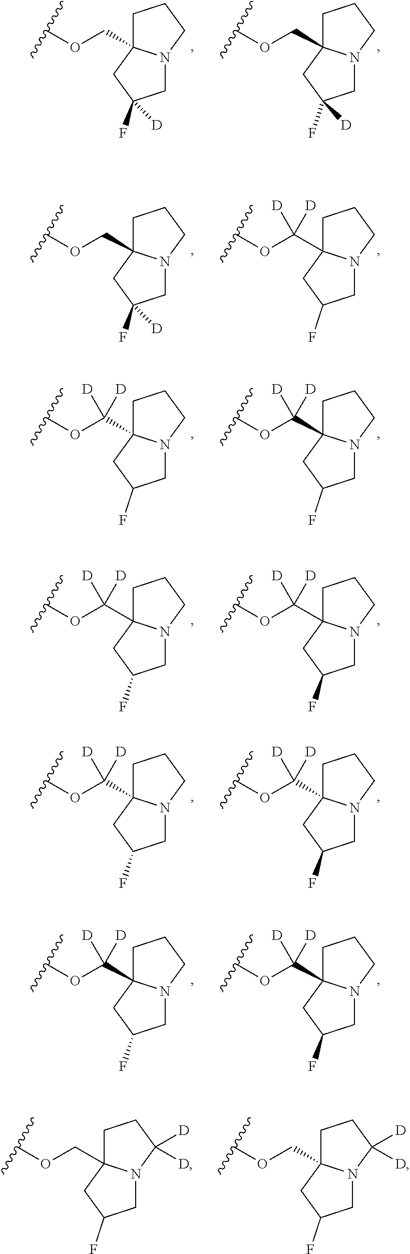

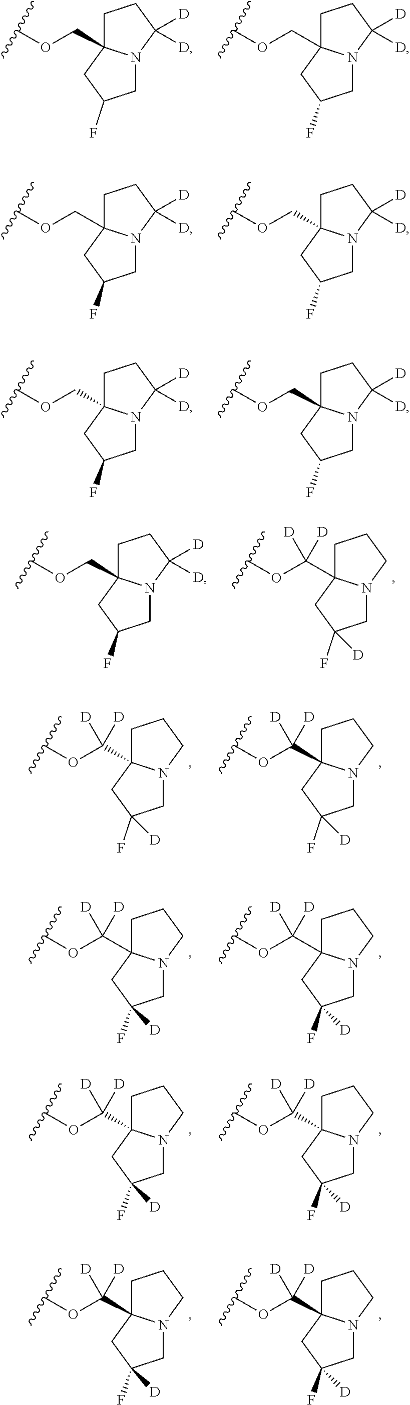

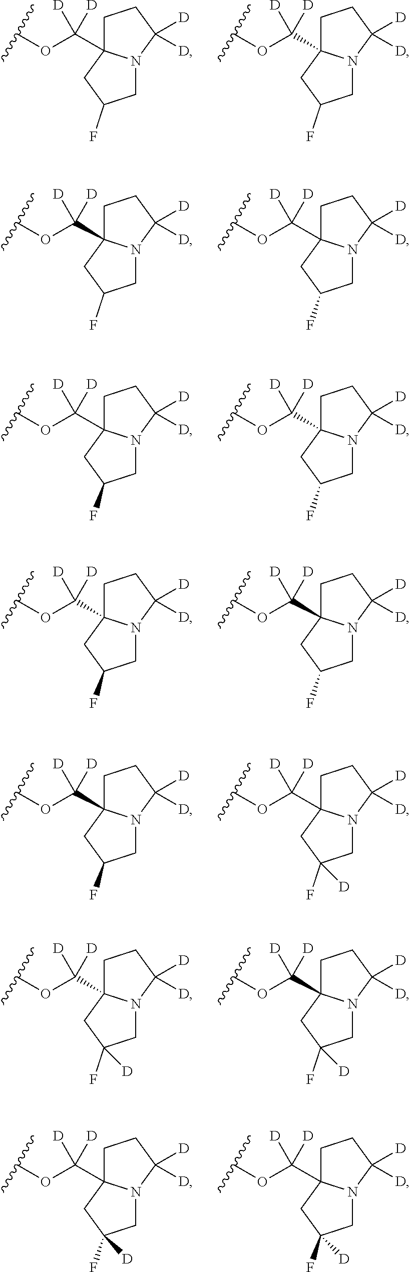

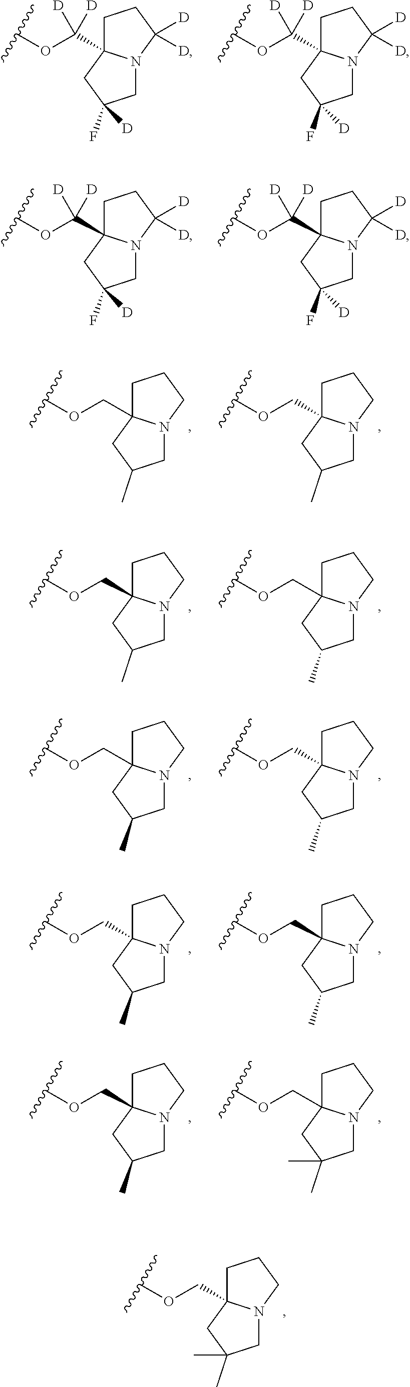

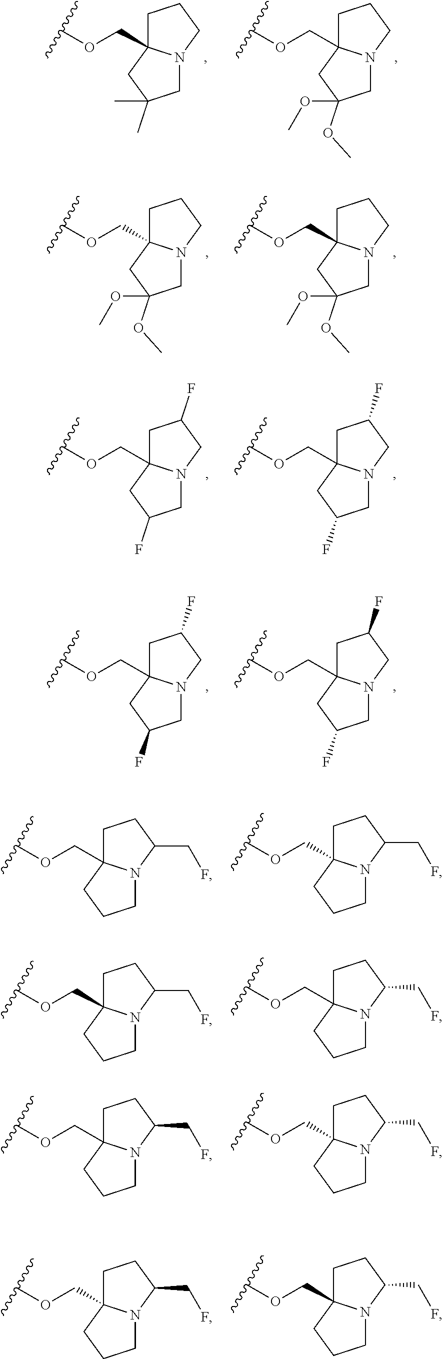

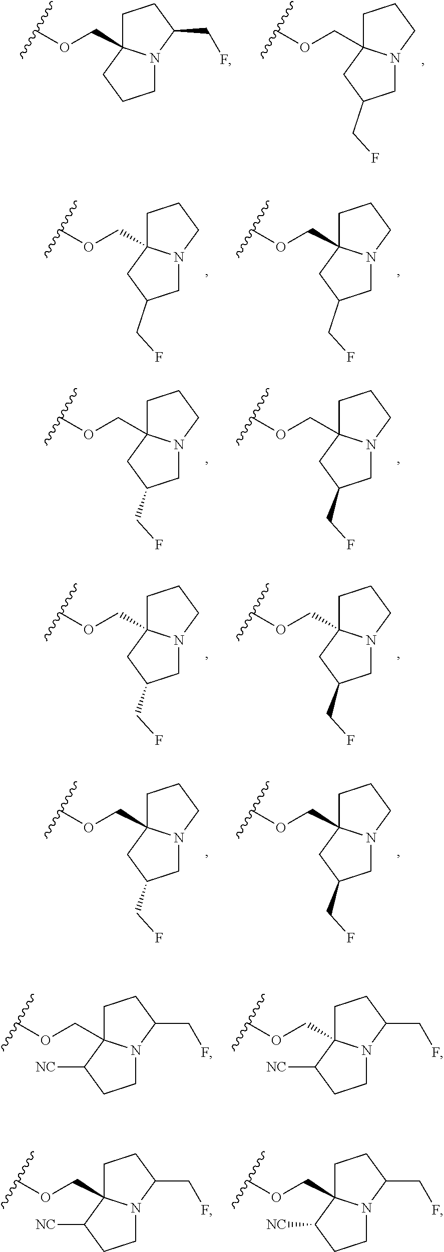

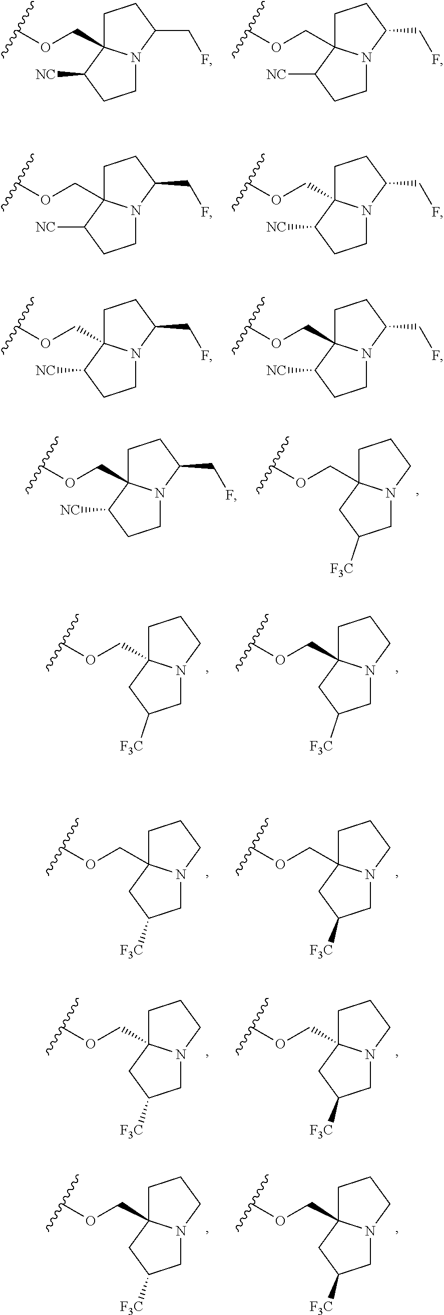

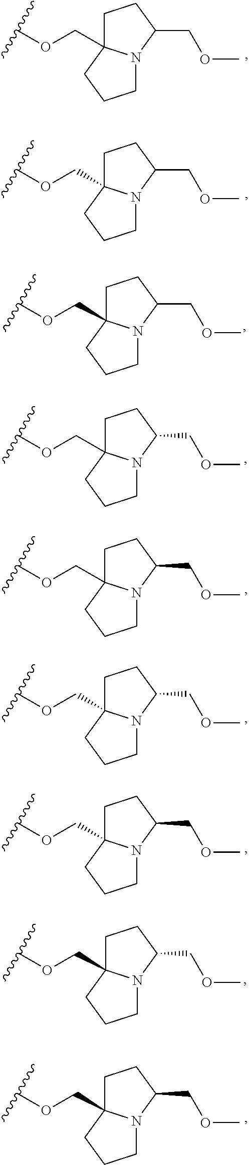

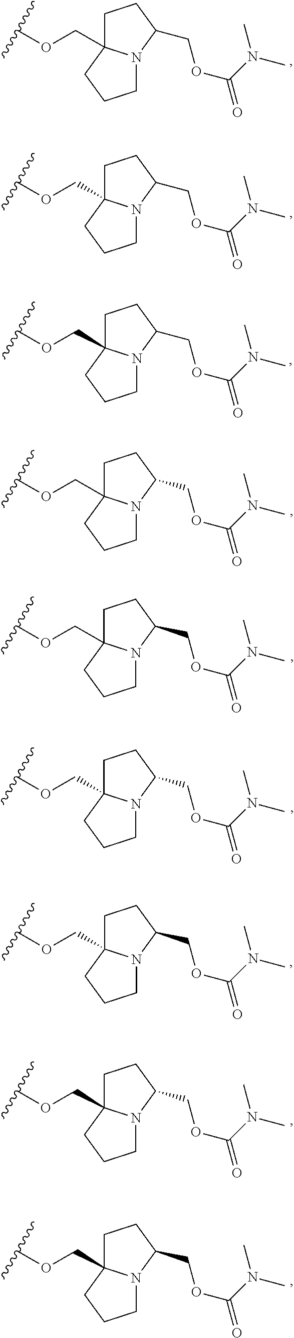

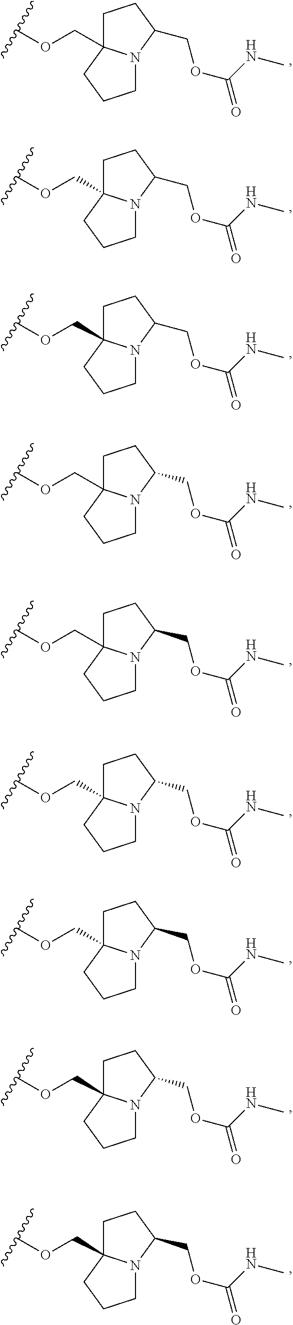

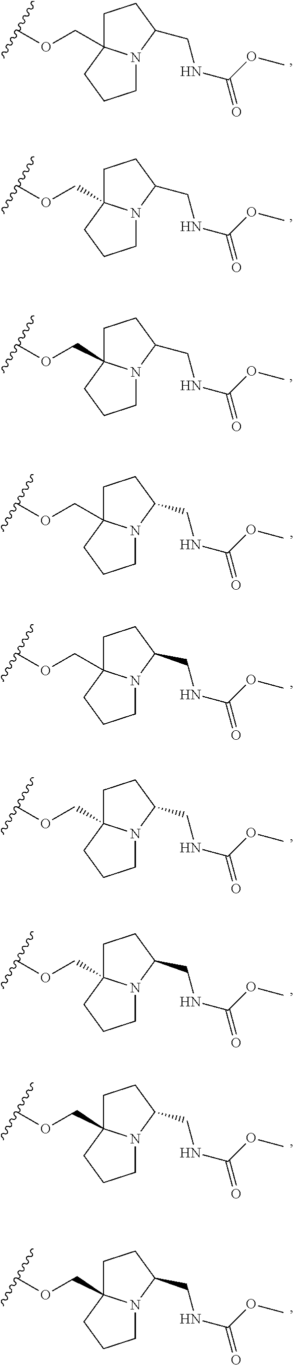

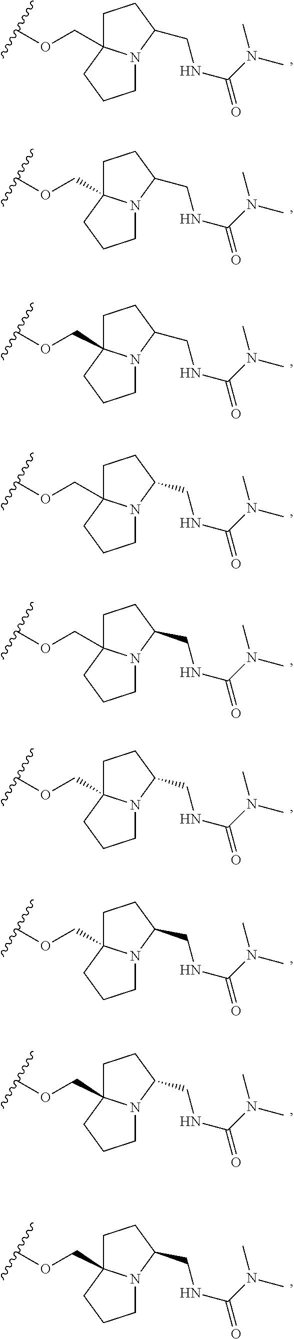

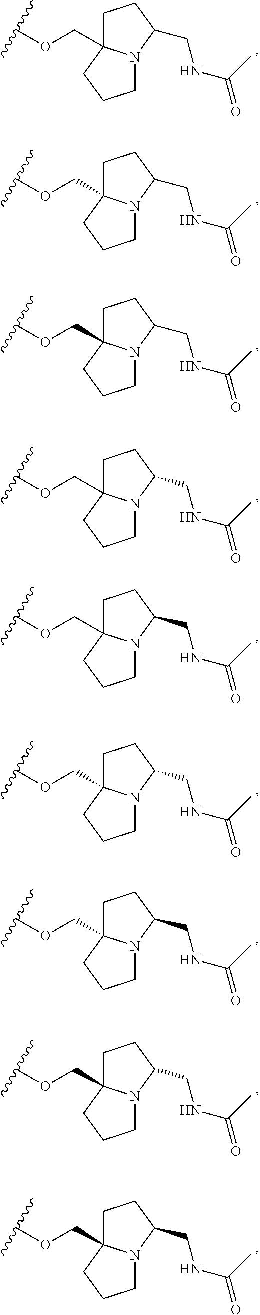

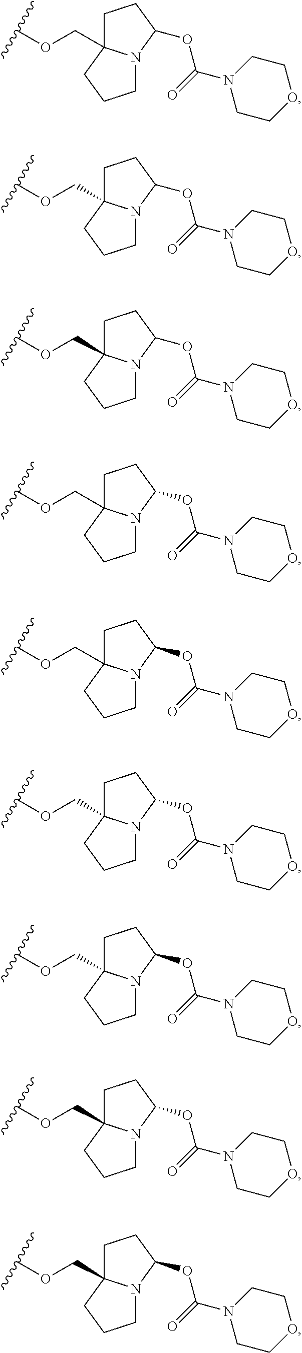

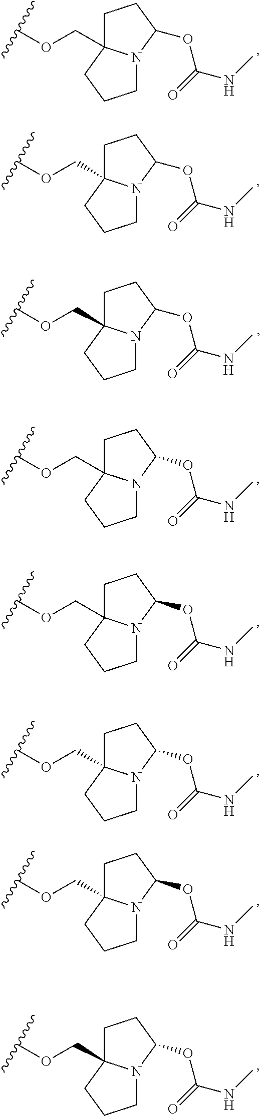

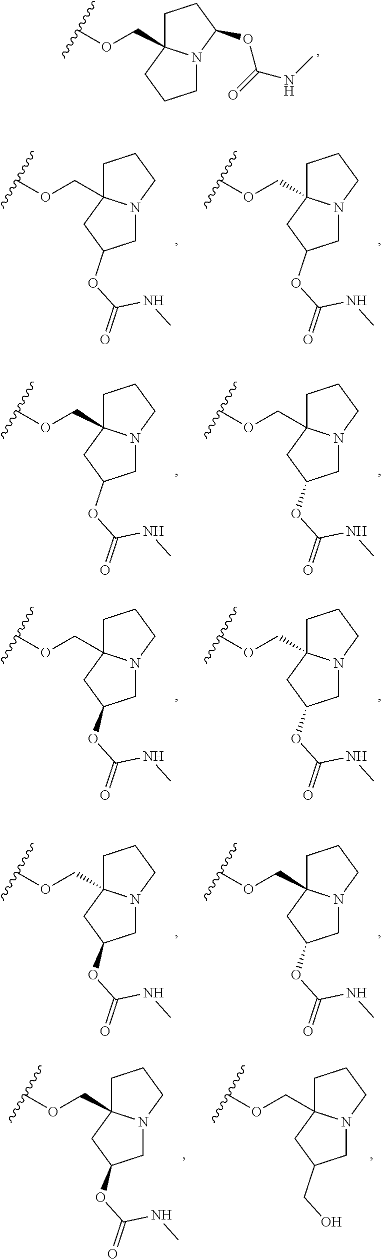

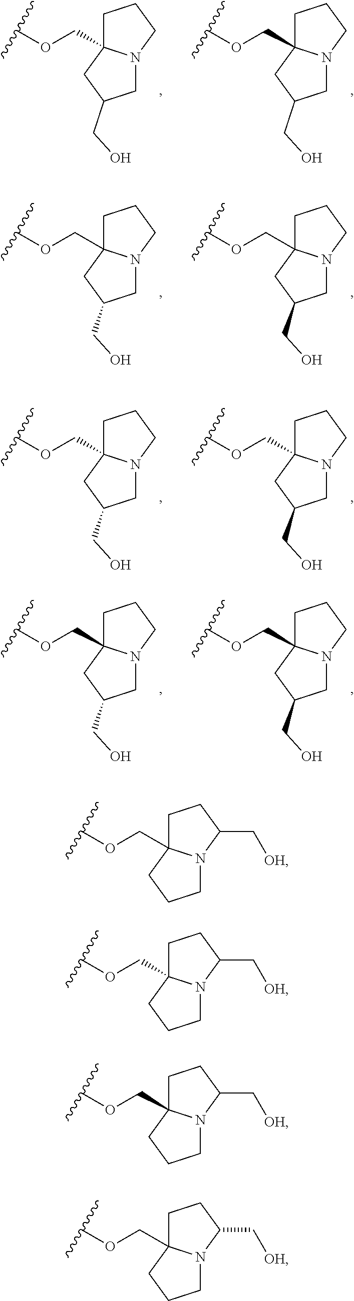

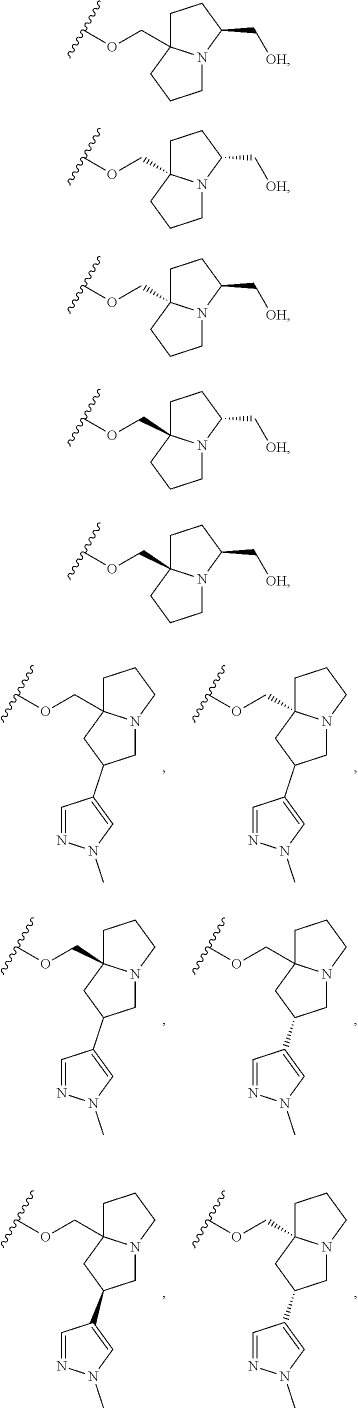

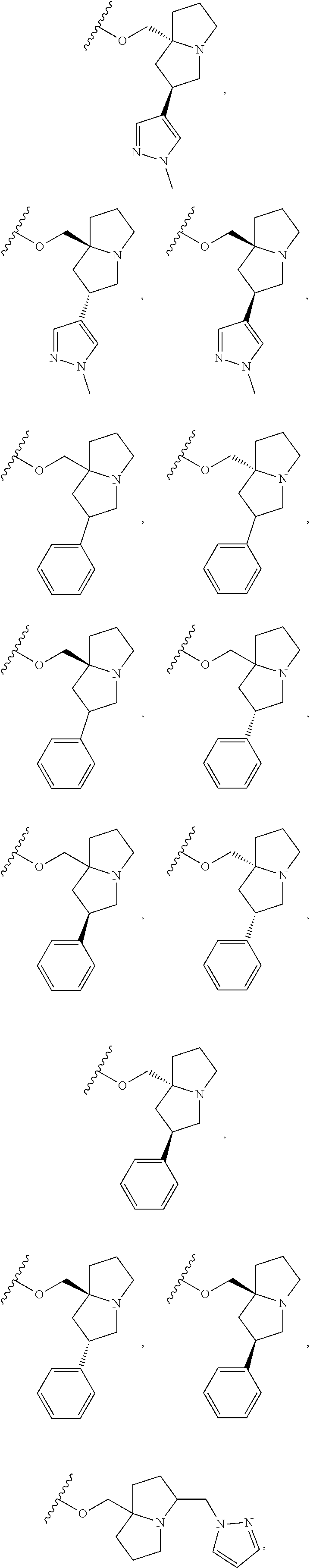

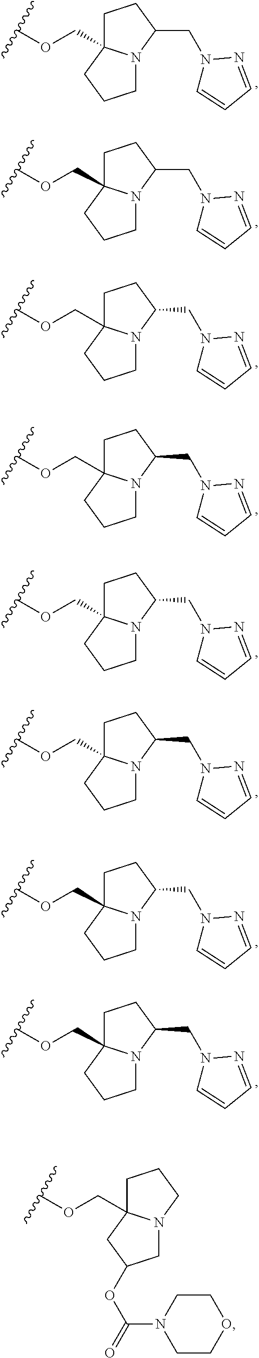

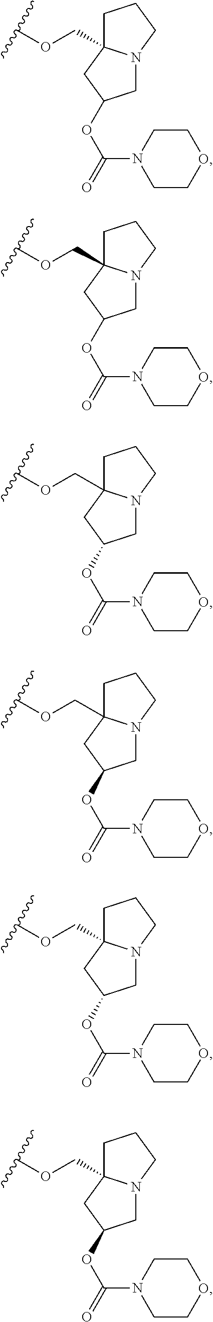

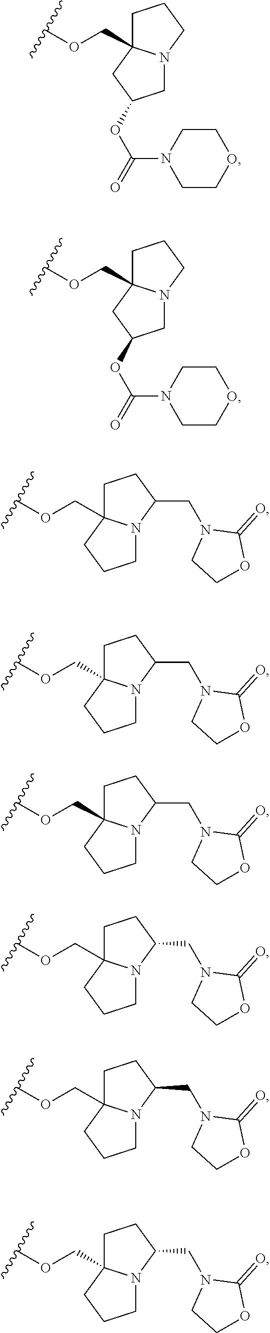

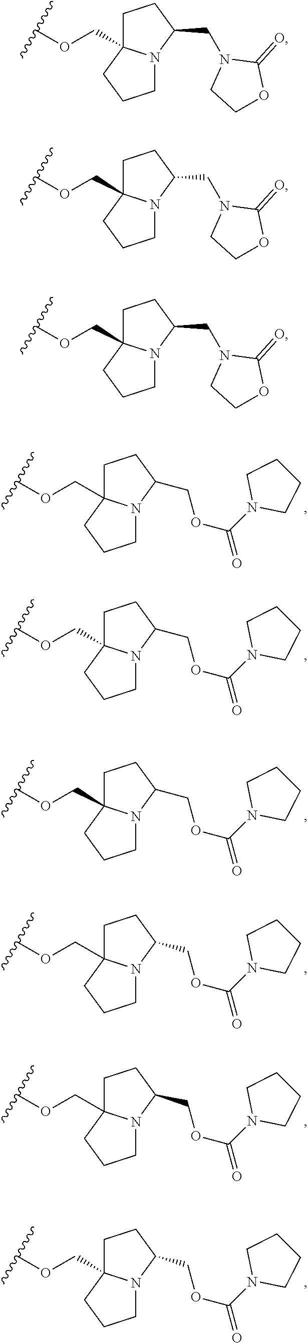

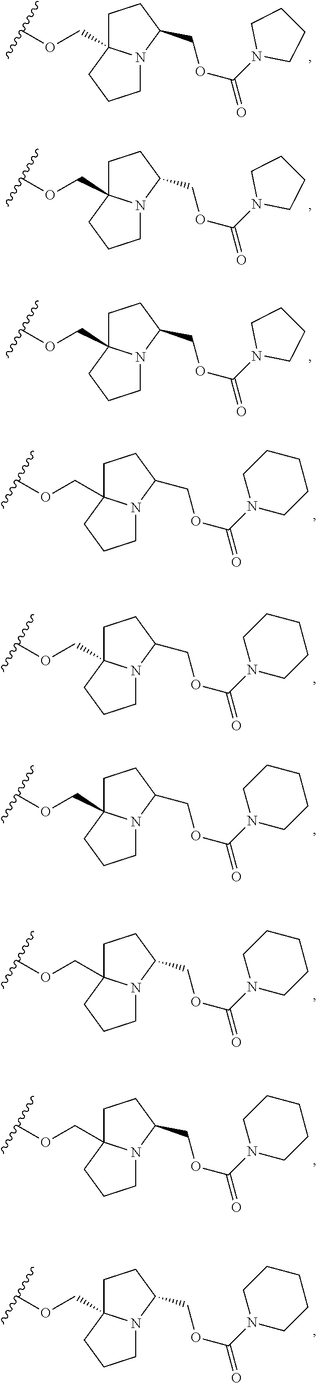

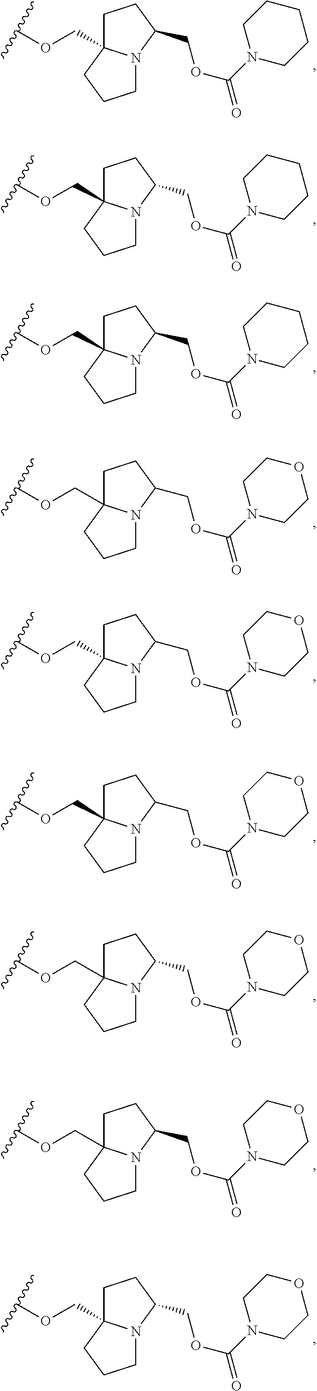

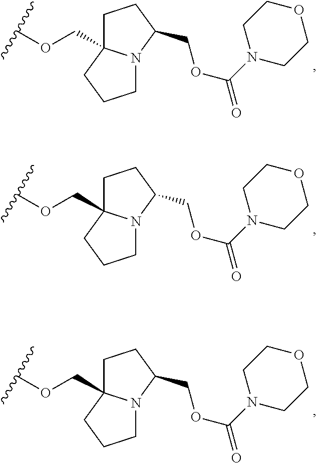 [SMILES: *OC[C@@]12CCCN1[C@H](COC(=O)N1CCOCC1)CC2 ; *OC[C@]12CCCN1[C@@H](COC(=O)N1CCOCC1)CC2 ; *OC[C@]12CCCN1[C@H](COC(=O)N1CCOCC1)CC2]

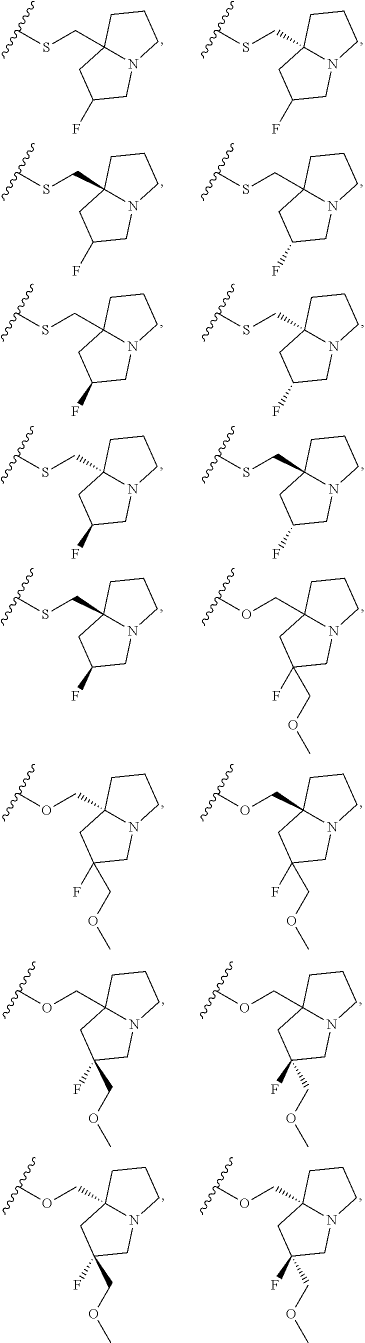

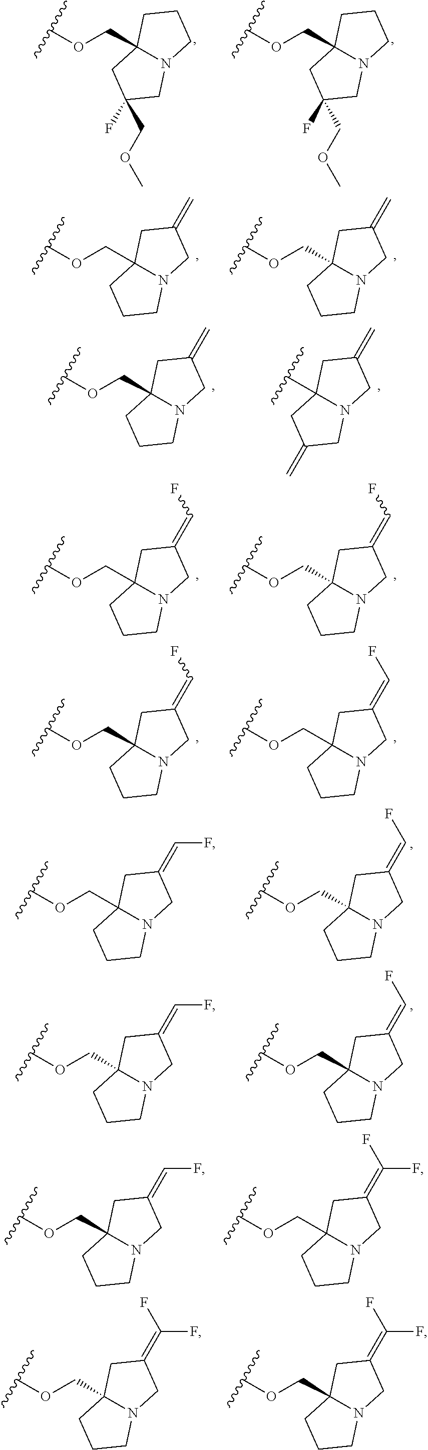

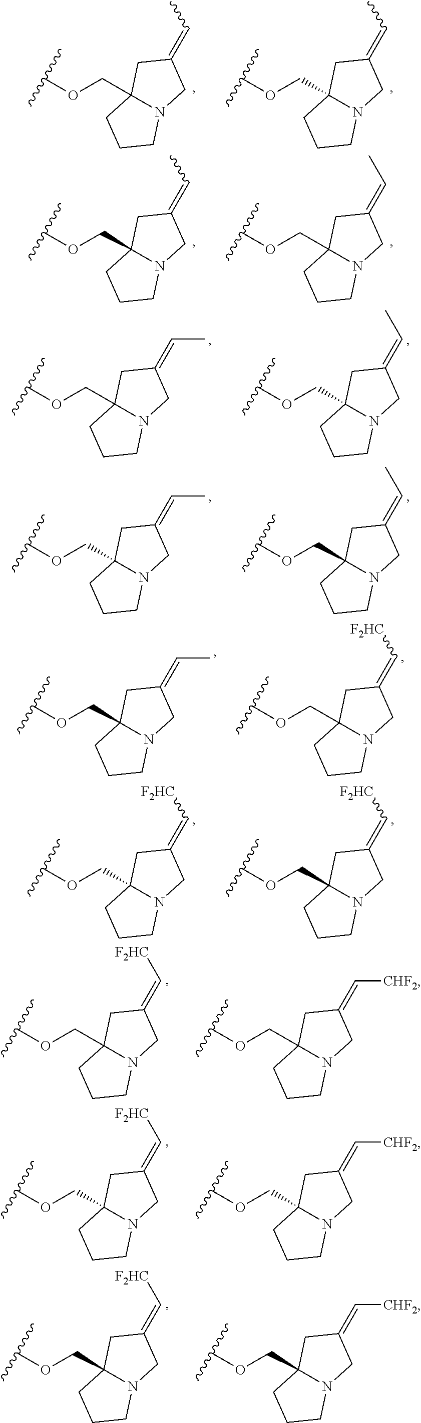

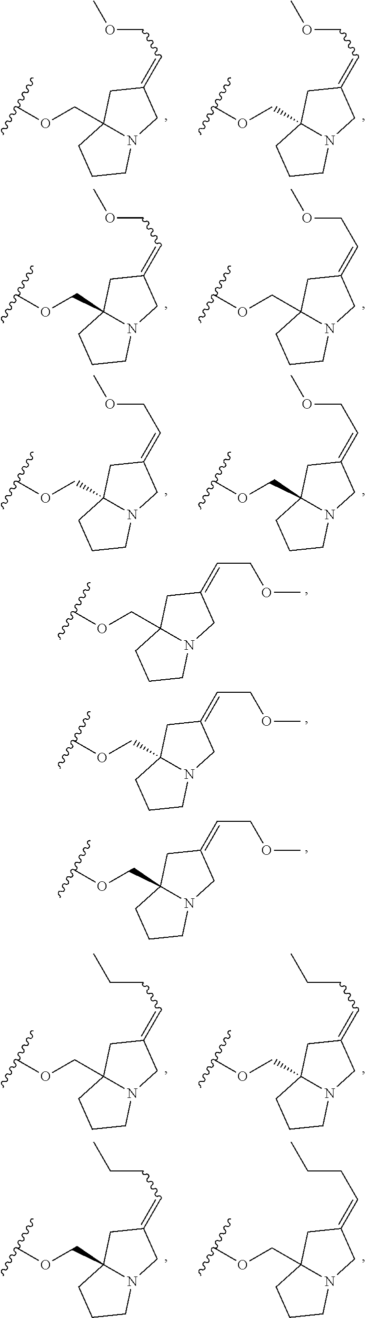

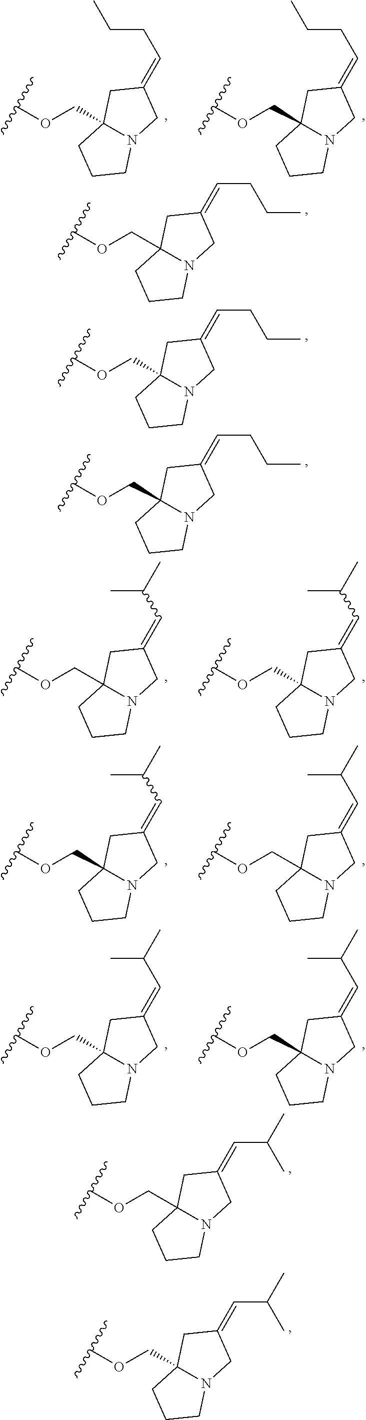

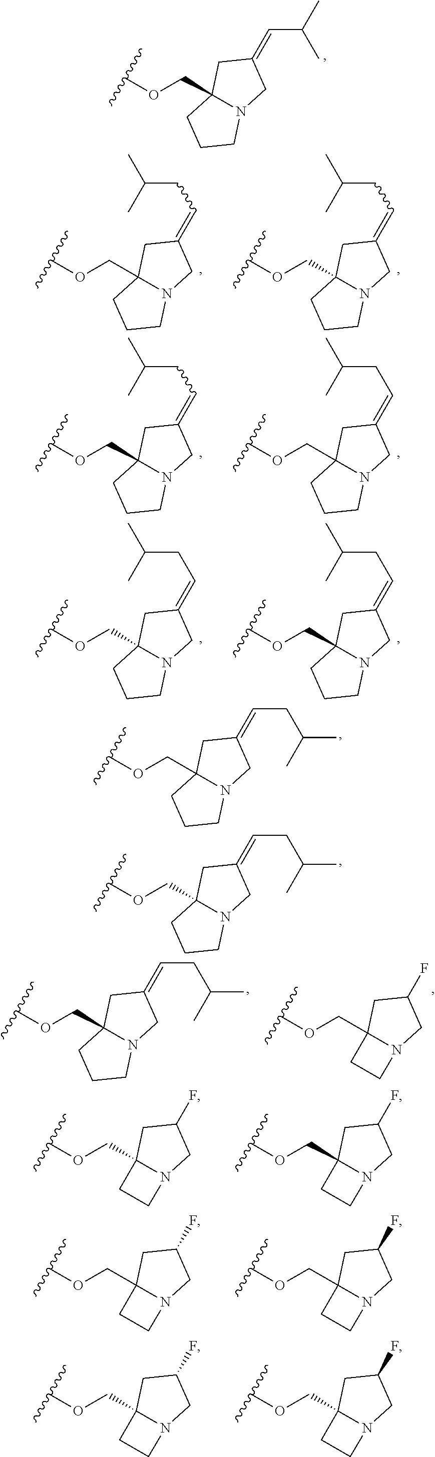

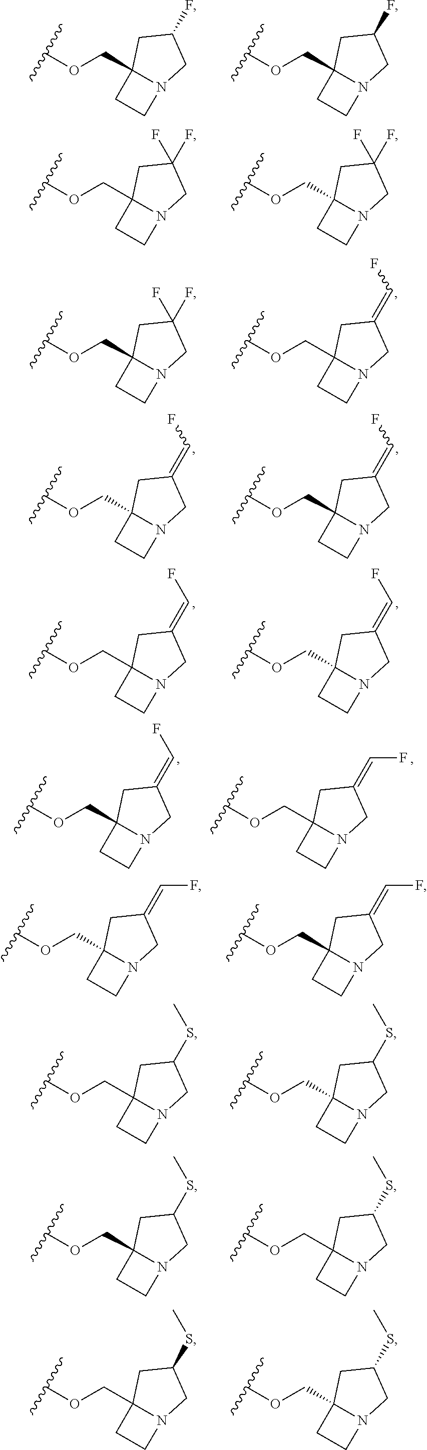

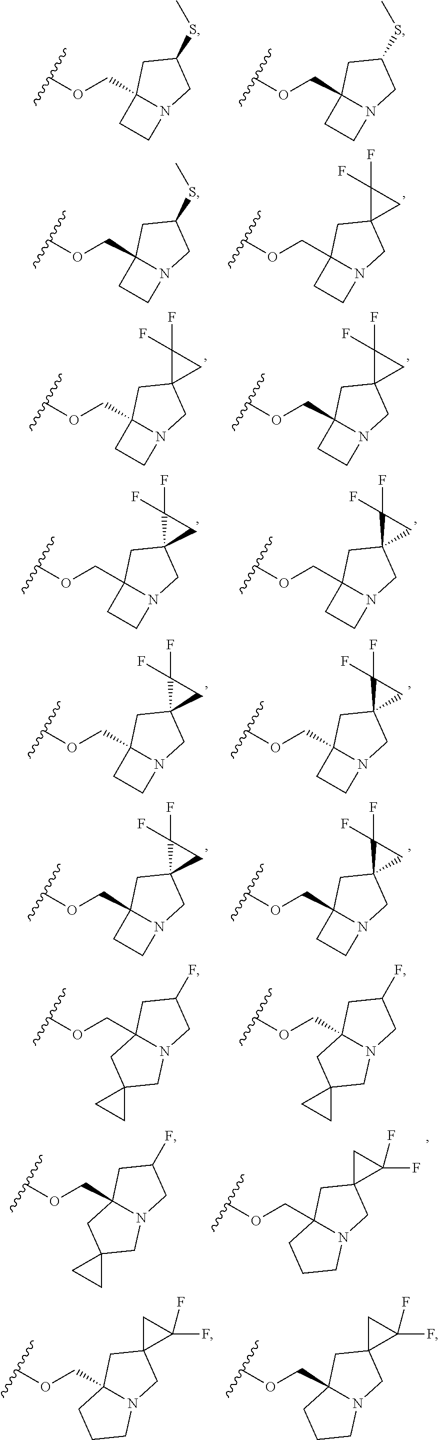

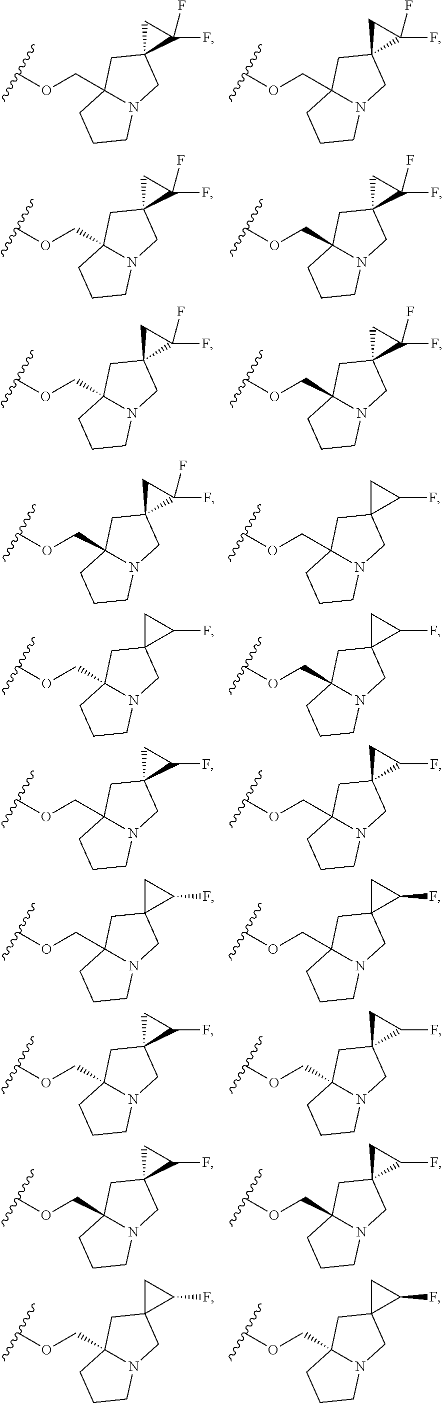

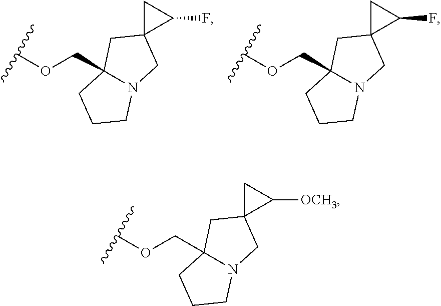 [SMILES: *OC[C@]12CCCN1CC1(C[C@@H]1F)C2 ; *OC[C@]12CCCN1CC1(C[C@H]1F)C2 ; *OCC12CCCN1CC1(CC1OC)C2]

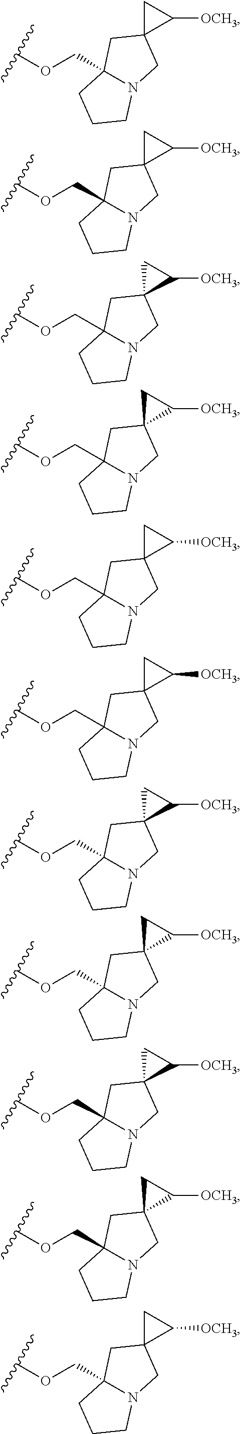

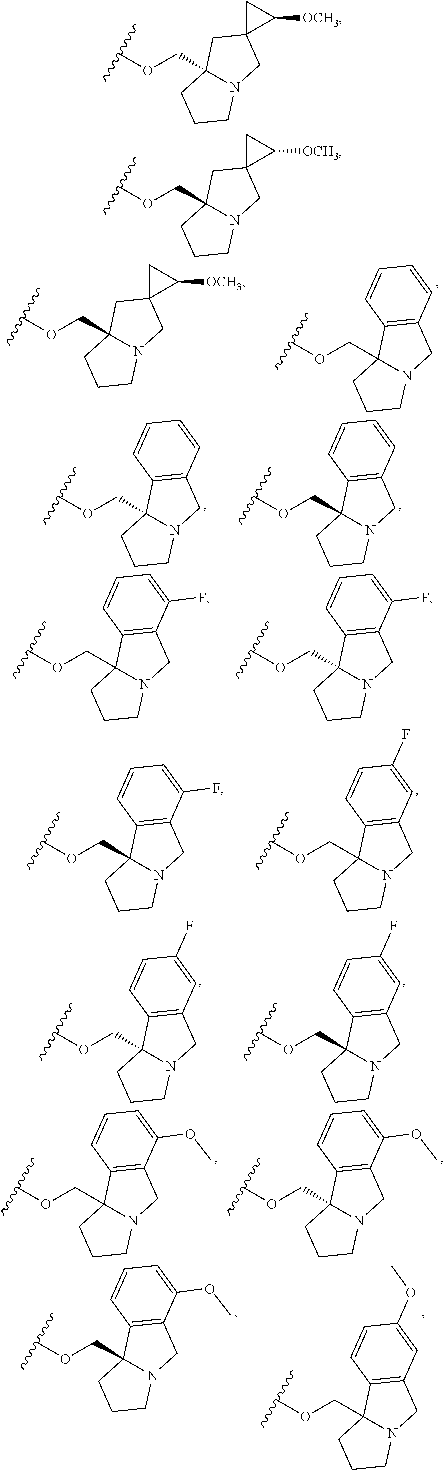

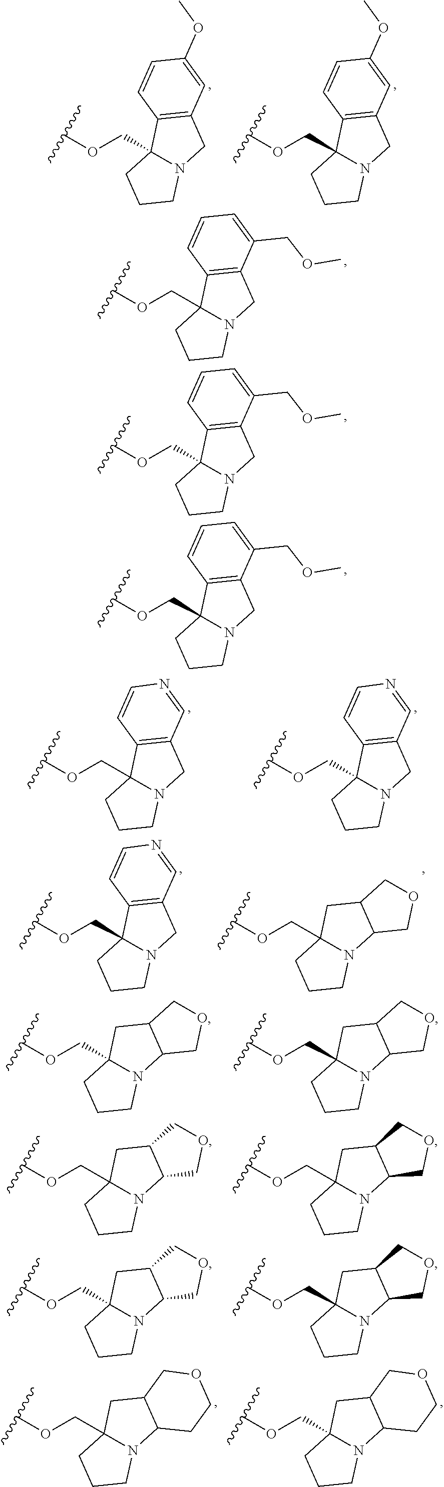

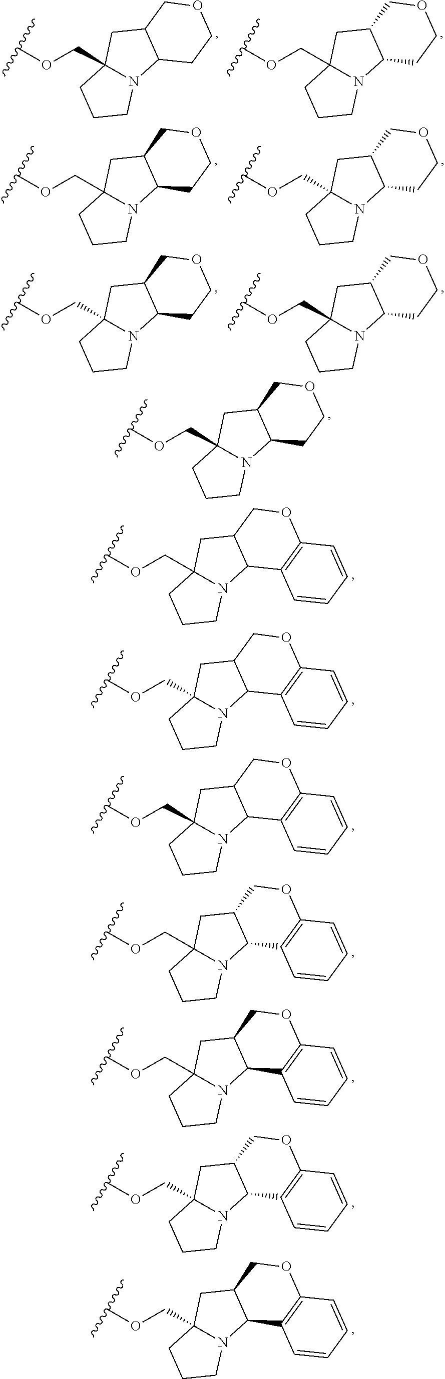

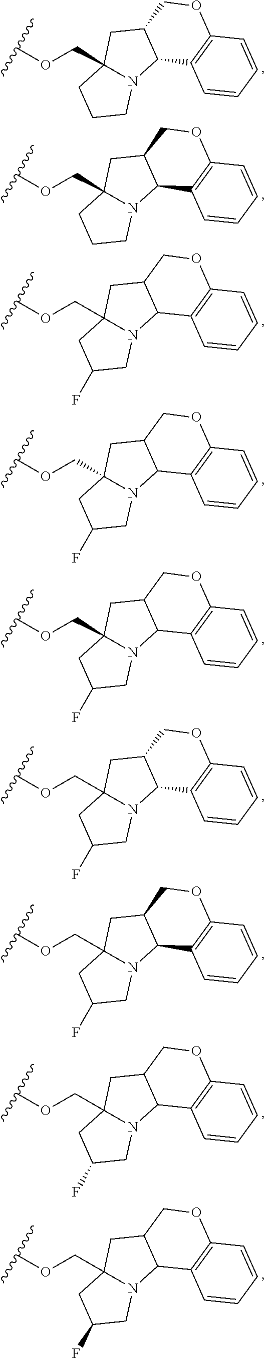

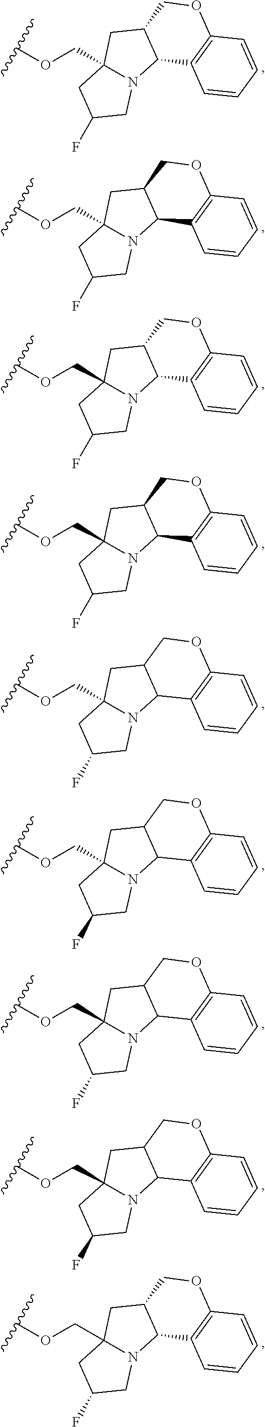

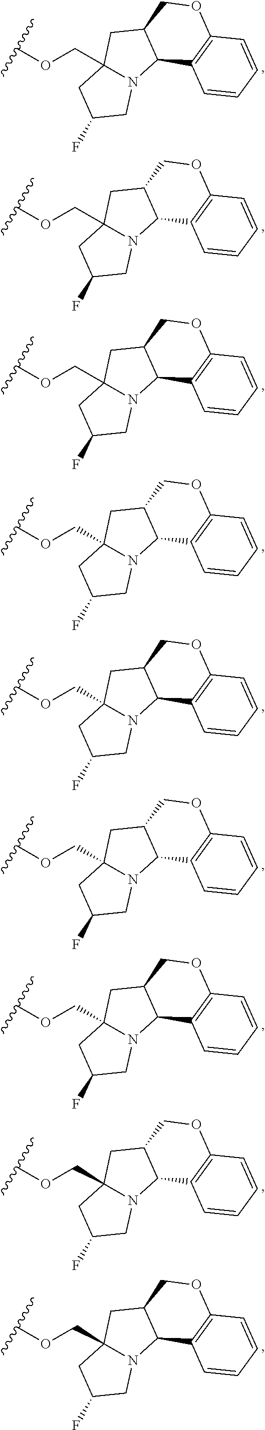

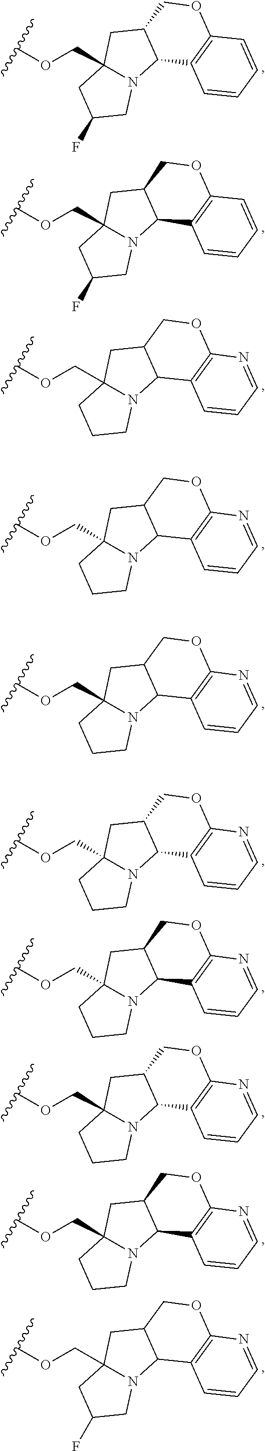

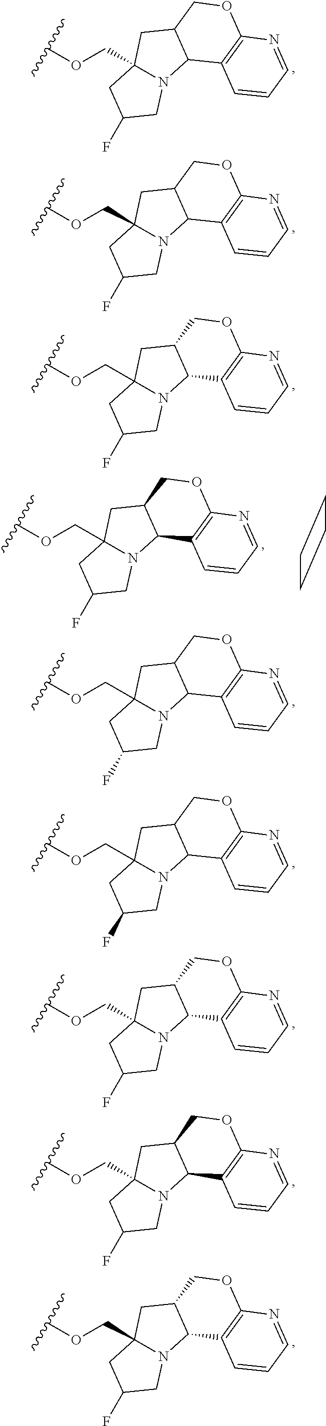

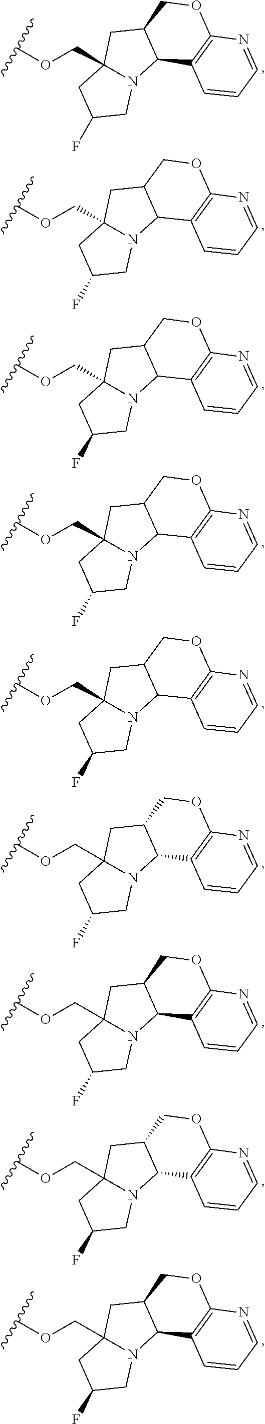

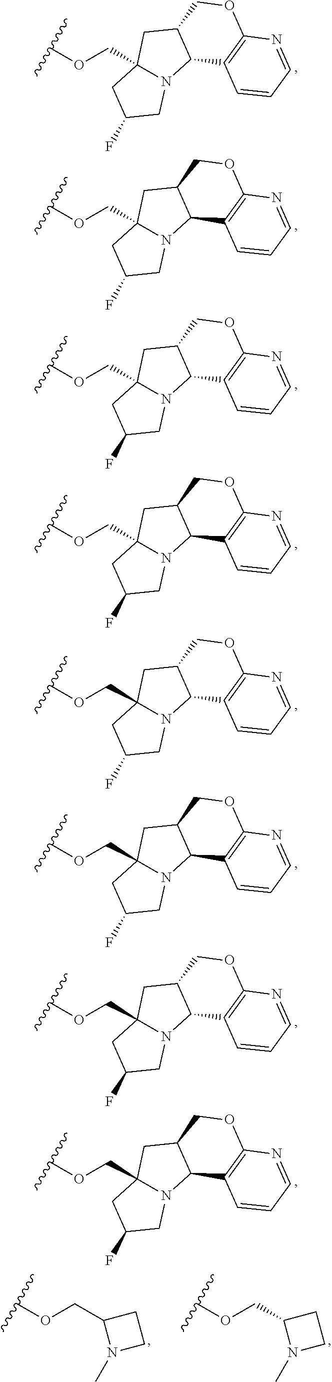

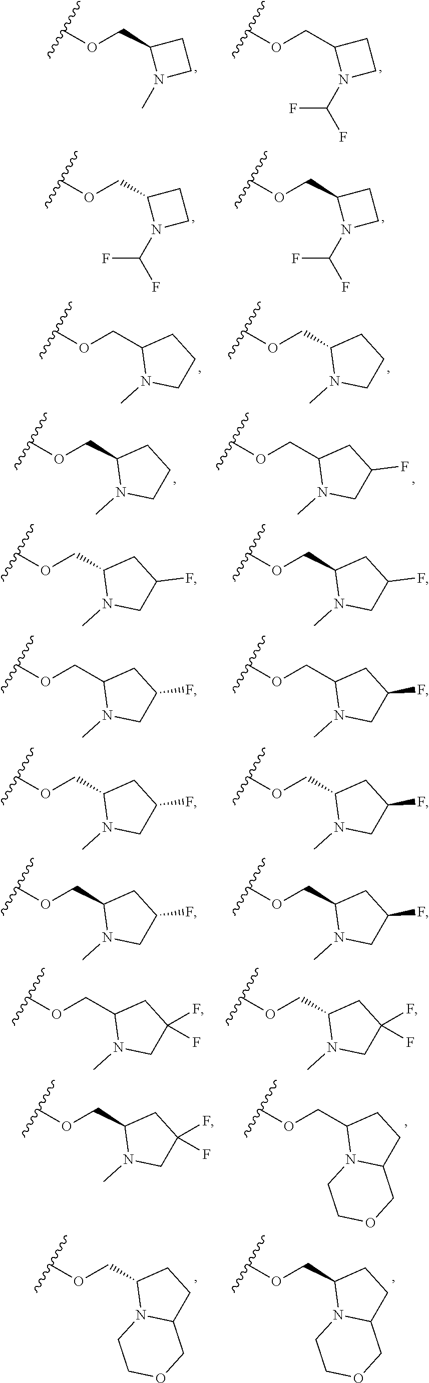

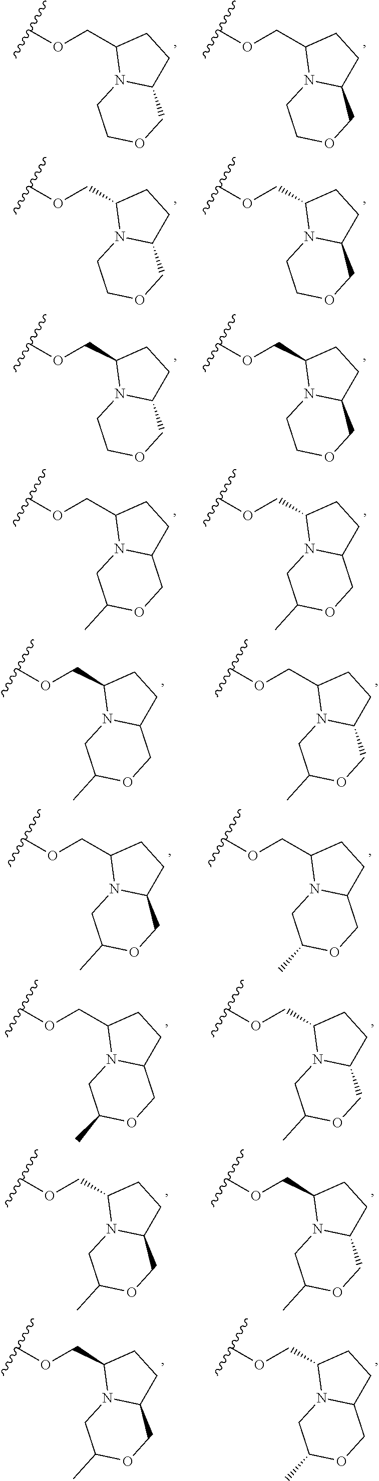

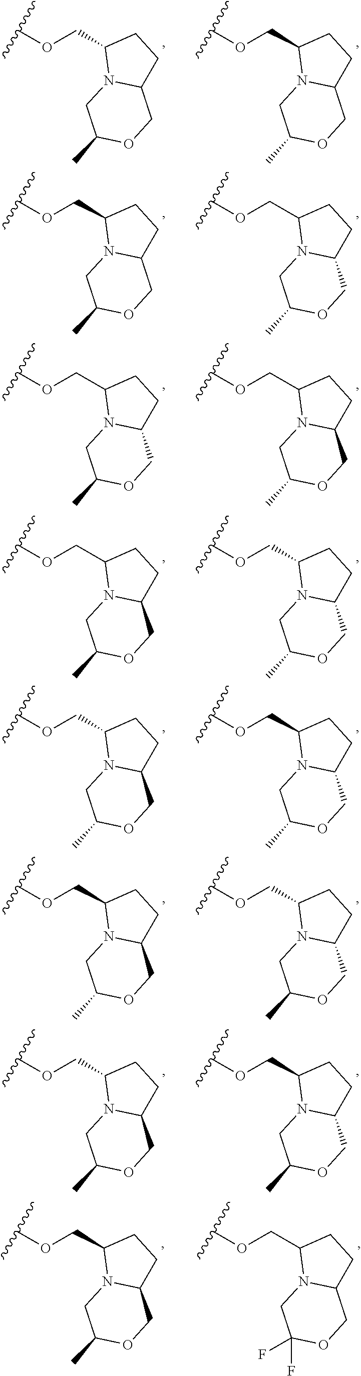

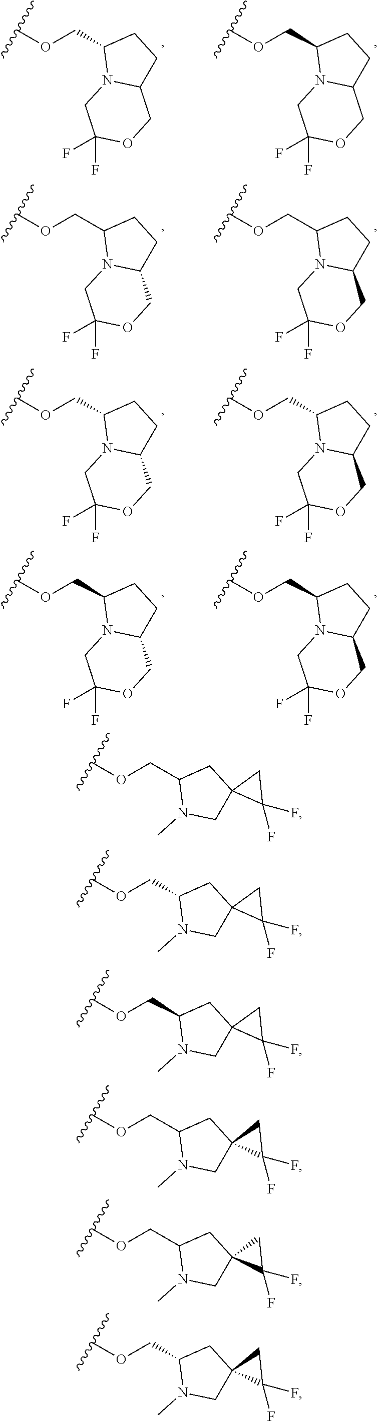

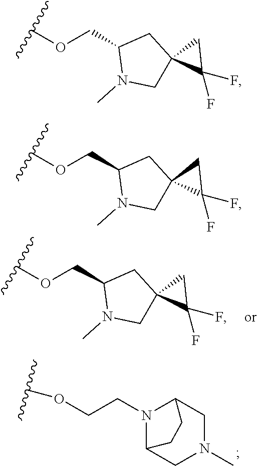 [SMILES: *OC[C@@H]1C[C@@]2(CN1C)CC2(F)F ; *OC[C@H]1C[C@]2(CN1C)CC2(F)F ; *OC[C@H]1C[C@@]2(CN1C)CC2(F)F ; *OCCN1C2CCC1CN(C)C2]

In some embodiments,

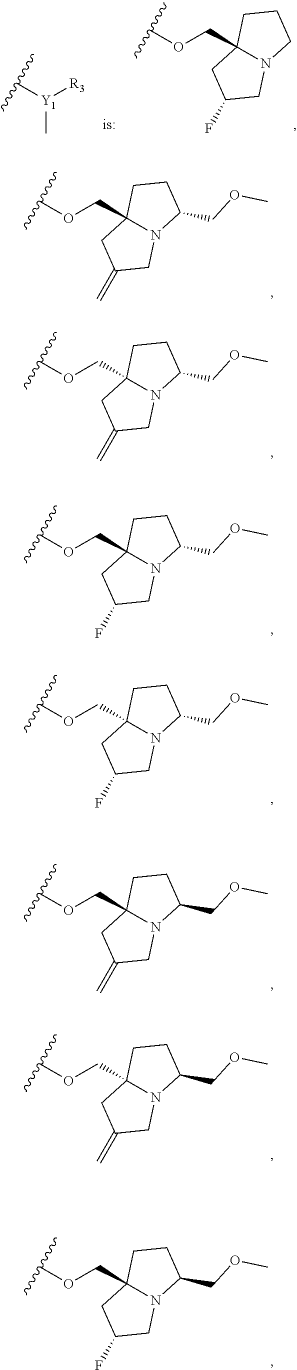

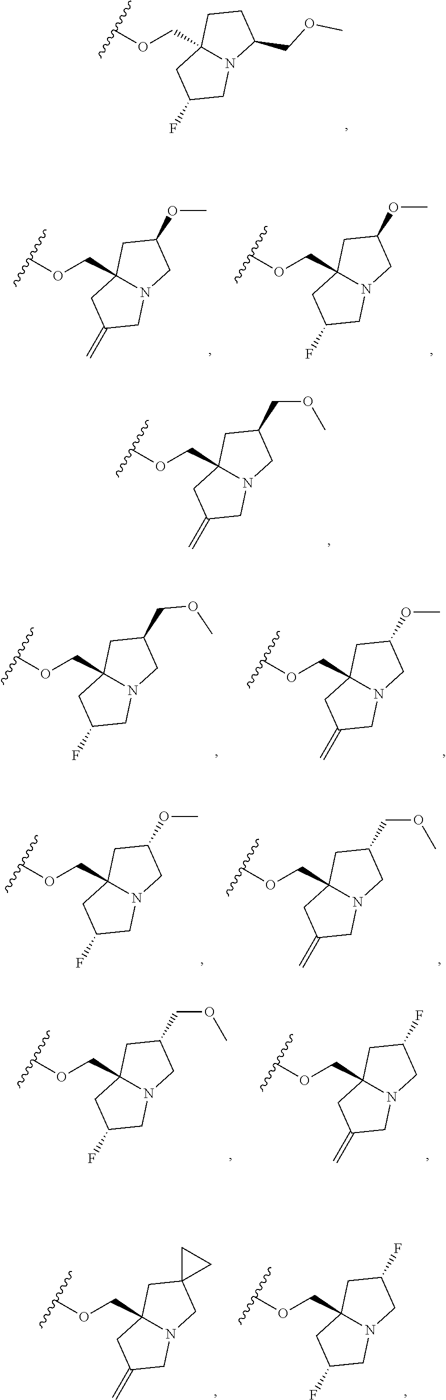

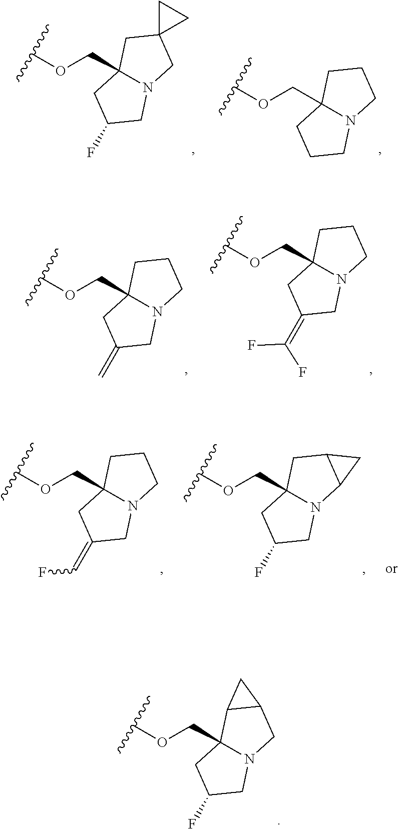

In some embodiments,

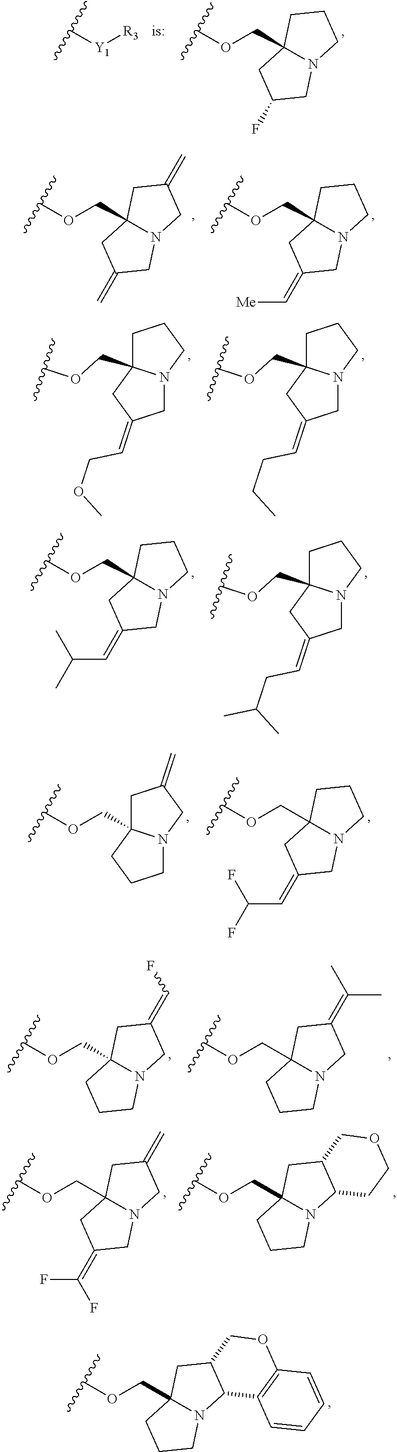

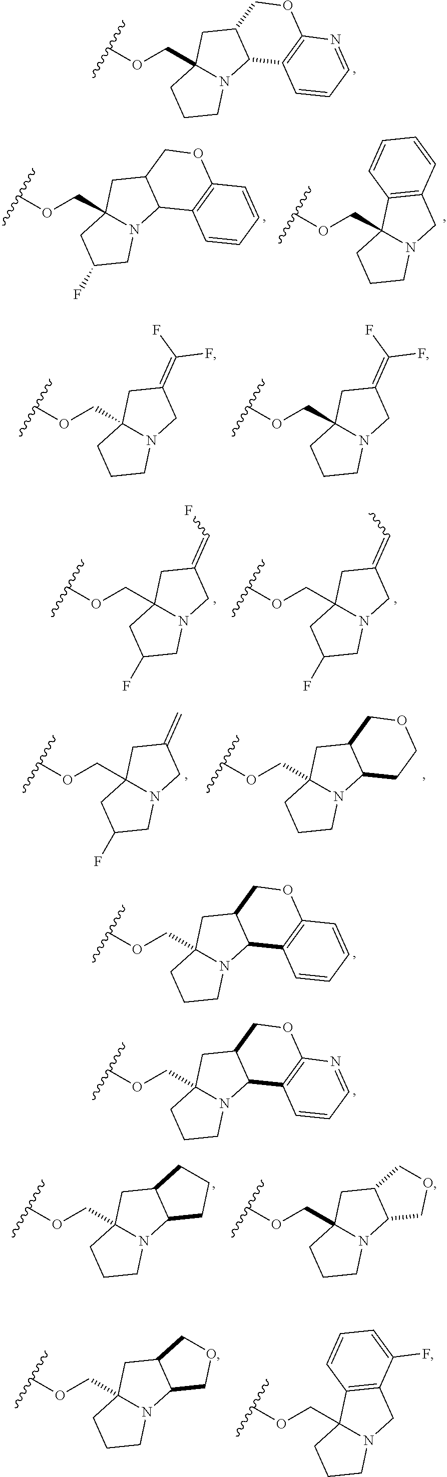

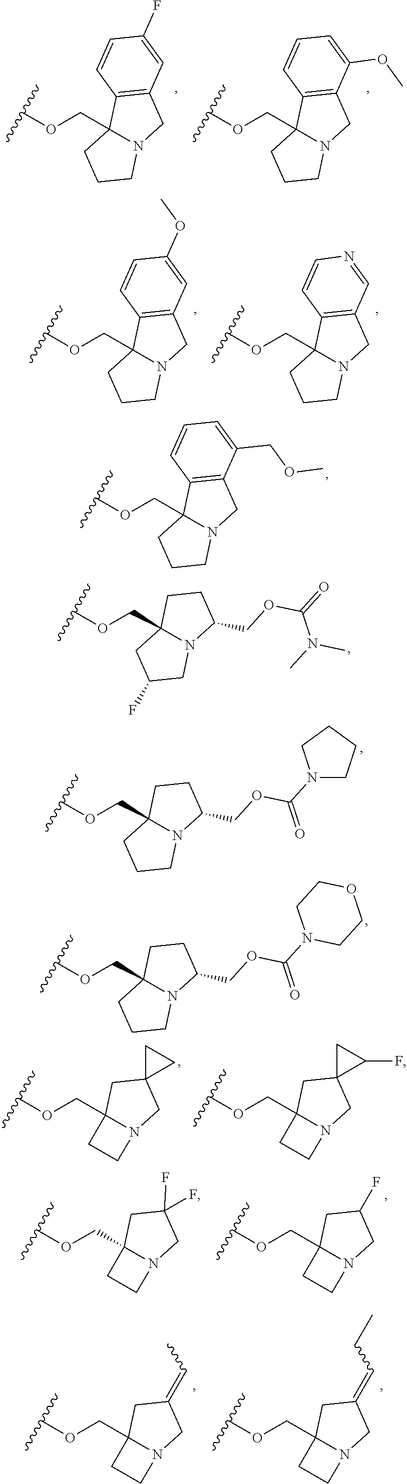

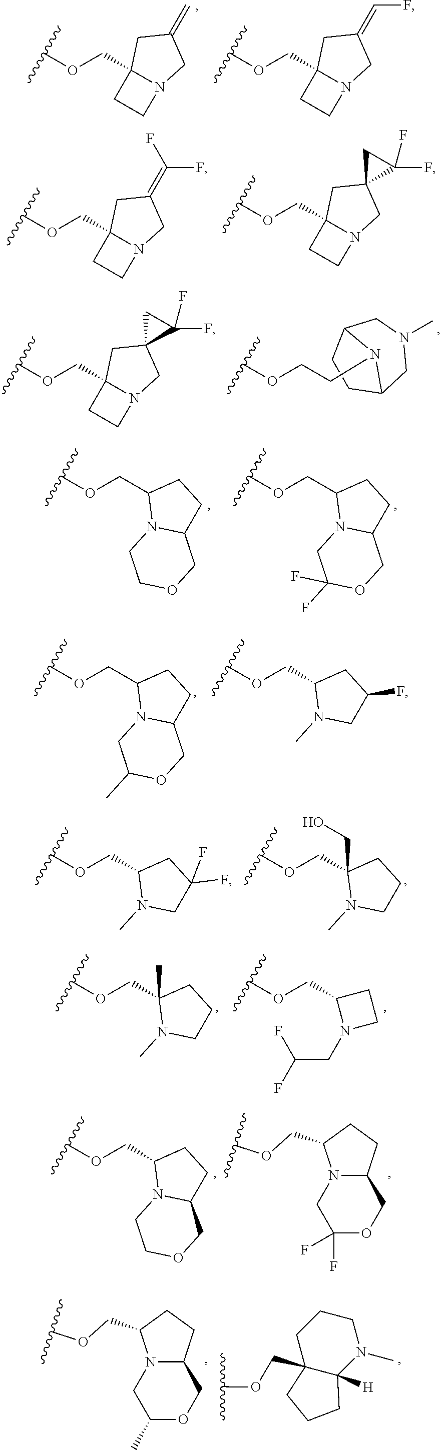

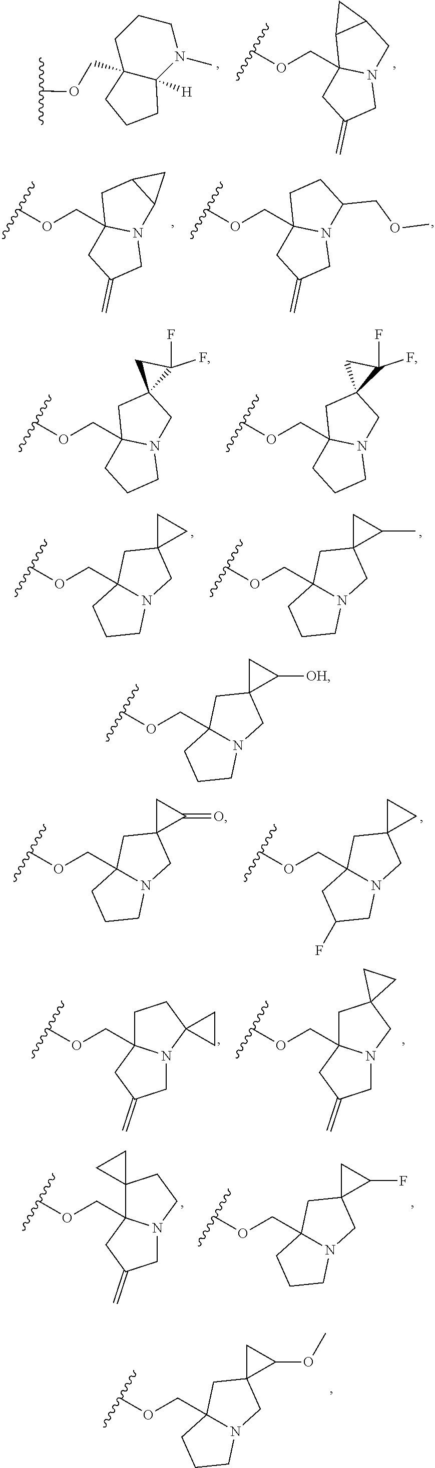

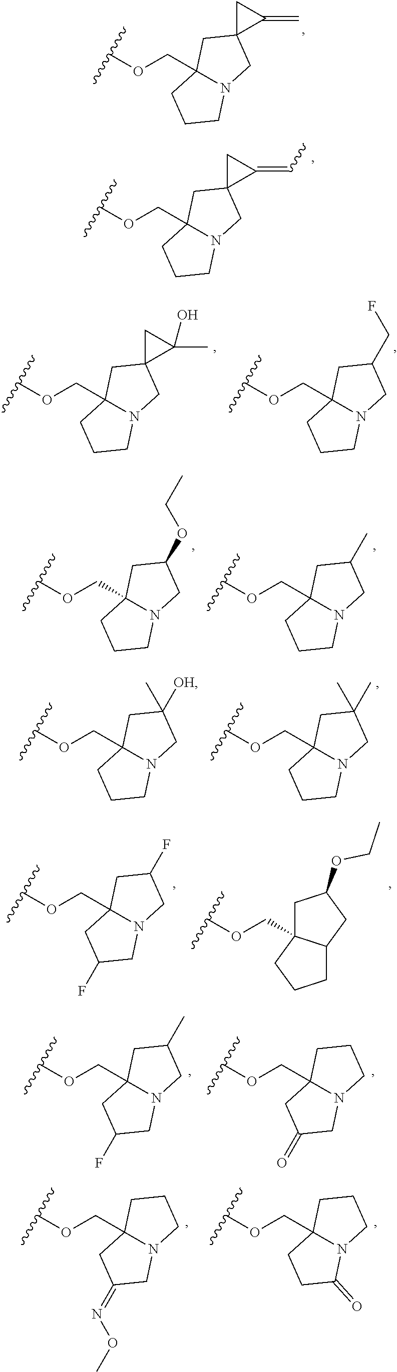

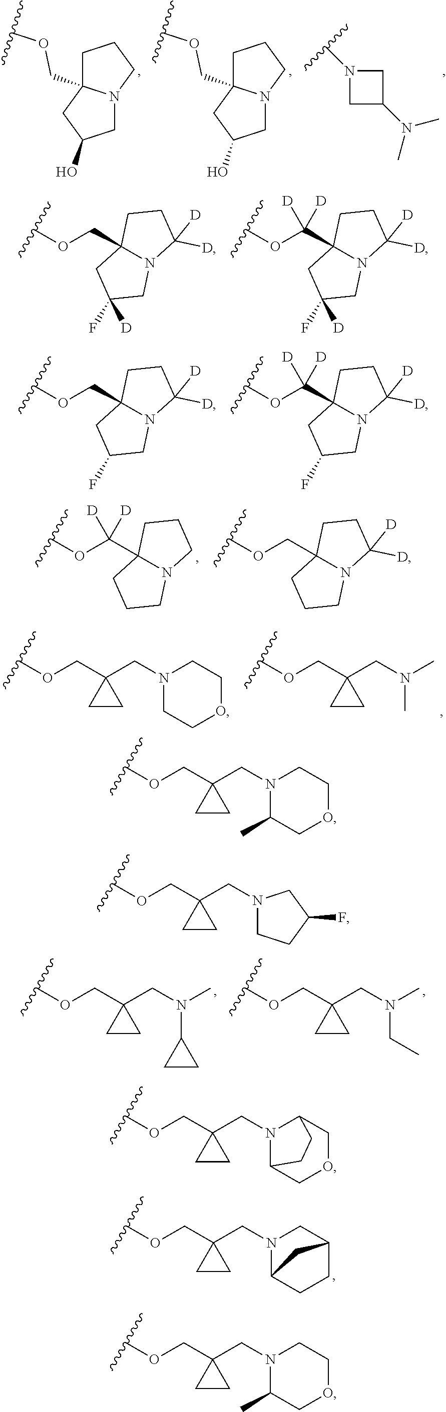

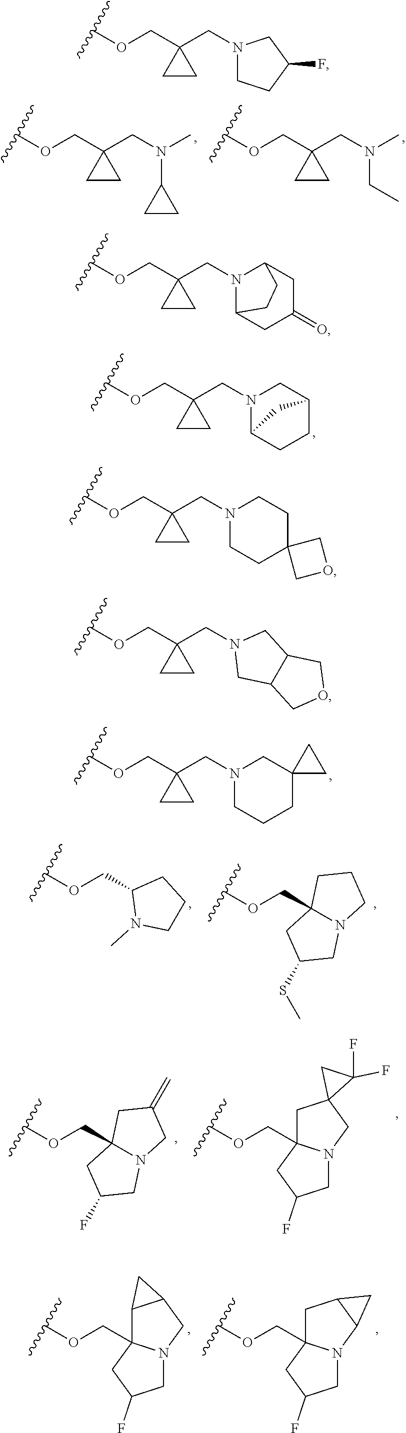

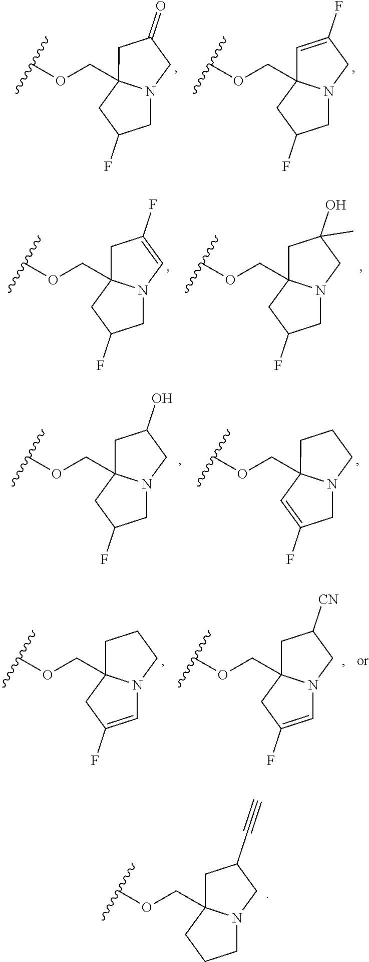

In some embodiments,

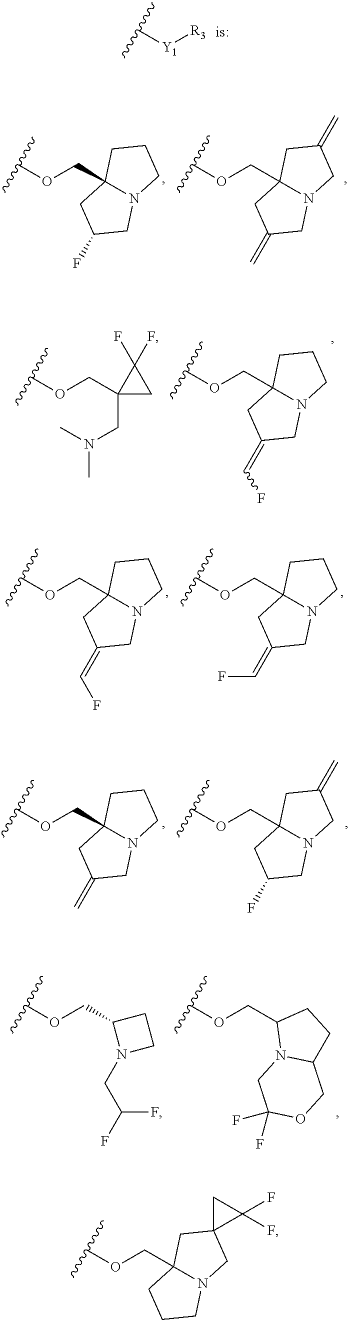

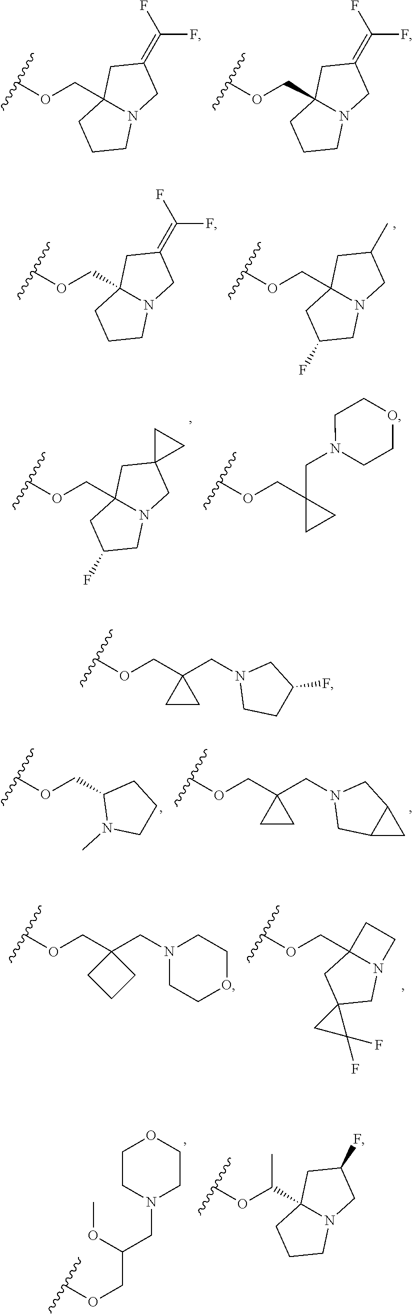

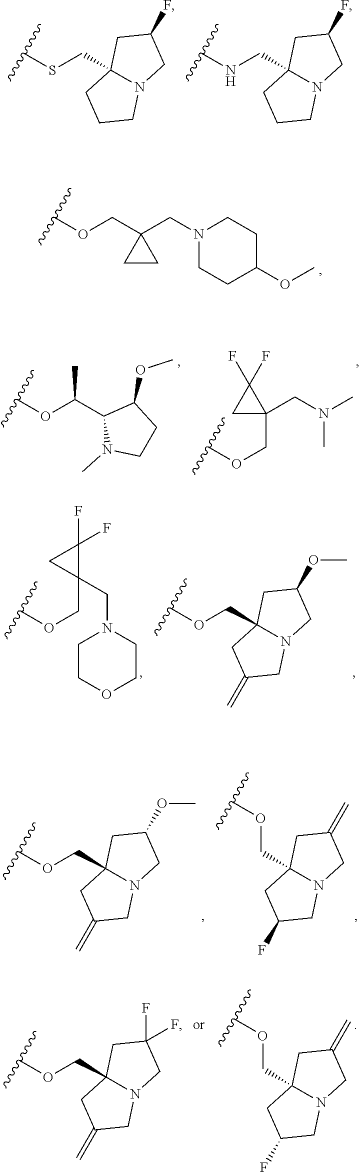

[60]. The compound of any one of [1] to [59], wherein, R₄ is phenyl, pyridyl, naphthyl, quinolyl, isoquinolyl, indazolyl, benzothiaphenyl or benzothiozolyl, said phenyl, pyridyl, naphthyl, quinolyl, isoquinolyl, indazolyl, benzothiaphenyl or benzothiozolyl is unsubstituted or substituted with 1, 2, 3, 4, 5 or 6 R_{S4}.

[61]. The compound of any one of [1] to [60], wherein R₄ is

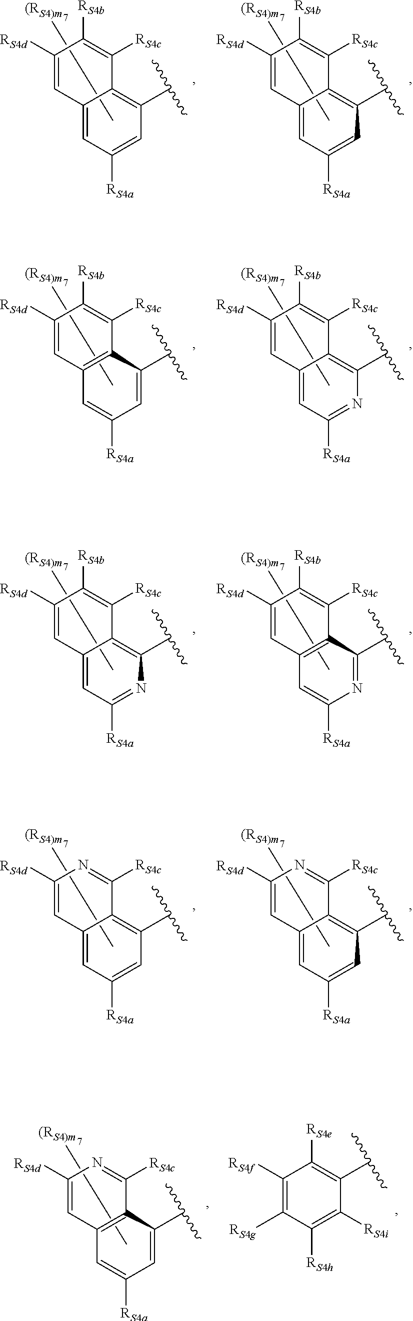

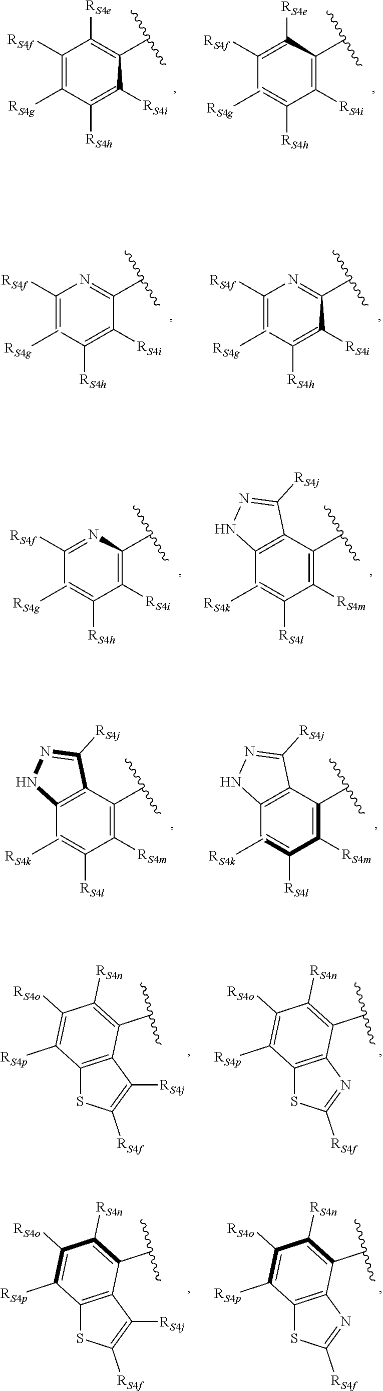

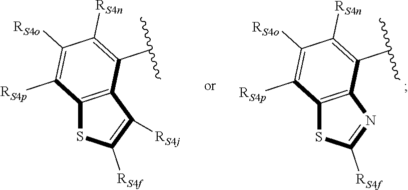

m₇ is 0, 1, 2, or 3;
R_{S4a} is —OH or —NH₂;
R_{S4b} is hydrogen, deuterium, or halogen;
R_{S4c} is hydrogen, deuterium, —C₁₋₃alkyl, —C₂₋₃alkenyl or —C₂₋₃alkynyl;
R_{S4d} is hydrogen, deuterium or halogen;
R_{S4e} is hydrogen, deuterium, halogen, —C₁₋₃alkyl or haloC₁₋₃alkyl;
R_{S4f} is —OH or —NH₂;
R_{S4g} is hydrogen, deuterium, halogen, —C₁₋₃alkyl or haloC₁₋₃alkyl;
R_{S4h} is hydrogen, deuterium, halogen, —C₁₋₃alkyl or haloC₁₋₃alkyl;
R_{S4i} is hydrogen, deuterium, halogen, —C₁₋₃alkyl or haloC₁₋₃alkyl;
R_{S4j} is hydrogen, deuterium, halogen, —CN, —C₁₋₃alkyl, haloC₁₋₃alkyl or —OhaloC₁₋₃alkyl;
R_{S4k} is hydrogen, deuterium, halogen, —CN, —C₁₋₃alkyl, haloC₁₋₃alkyl or —OhaloC₁₋₃alkyl;
R_{S4l} is hydrogen, deuterium, halogen, —CN, —C₁₋₃alkyl, haloC₁₋₃alkyl or —OhaloC₁₋₃alkyl;
R_{S4m} is hydrogen, deuterium, halogen, —CN, —C₁₋₃alkyl, haloC₁₋₃alkyl or —OhaloC₁₋₃alkyl;
R_{S4n} is hydrogen, deuterium, halogen, —C₁₋₃alkyl or haloC₁₋₃alkyl;
R_{S4o} is hydrogen, deuterium, halogen, —C₁₋₃alkyl or haloC₁₋₃alkyl;
R_{S4p} is hydrogen, deuterium, halogen, —C₁₋₃alkyl or haloC₁₋₃alkyl.

[62]. The compound of [61], wherein:
m₇ is 0;
R_{S4a} is —OH or —NH₂;
R_{S4b} is —F;
R_{S4c} is ethyl, ethenyl or ethynyl;
R_{S4d} is hydrogen, or —F;
R_{S4e} is —F;
R_{S4f} is —NH₂;
R_{S4g} is hydrogen, —F, or methyl;
R_{S4h} is hydrogen, —F or methyl;
R_{S4i} is —I or —CF₃;
R_{S4j} is —CN;
R_{S4k} is hydrogen;
R_{S4l} is methyl;
R_{S4m} is —CF₃, —OCF₂Cl, —OCF₃ or —CF₂H;
R_{S4n} is hydrogen, —F, or methyl;
R_{S4o} is hydrogen, —F or methyl;
R_{S4p} is hydrogen, —F, or methyl.

[63]. The compound of any one of [1] to [62], wherein:
R₄ is

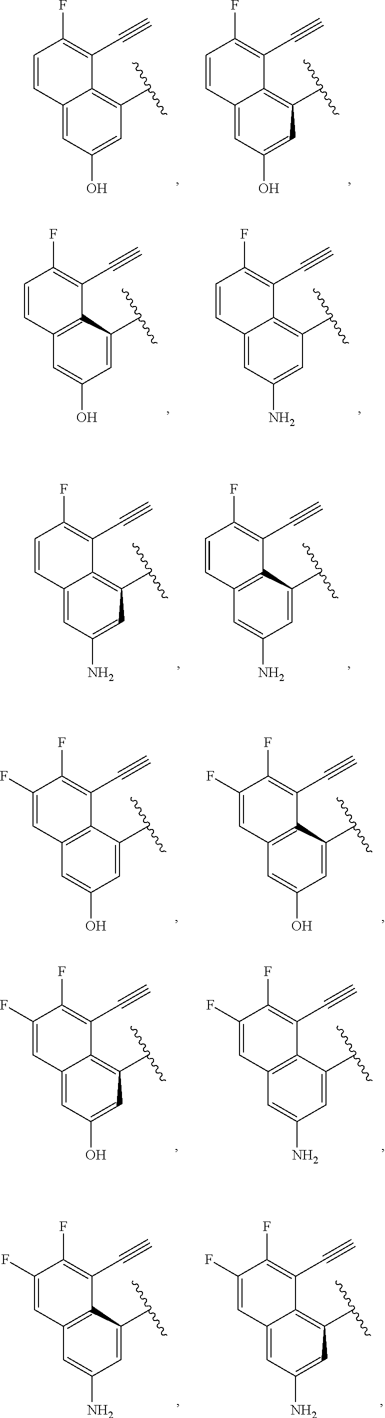

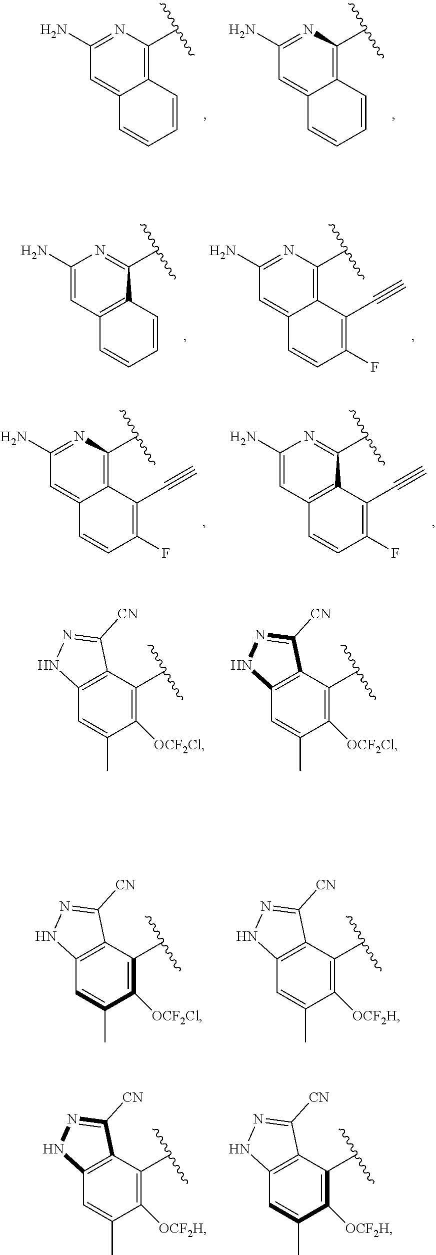

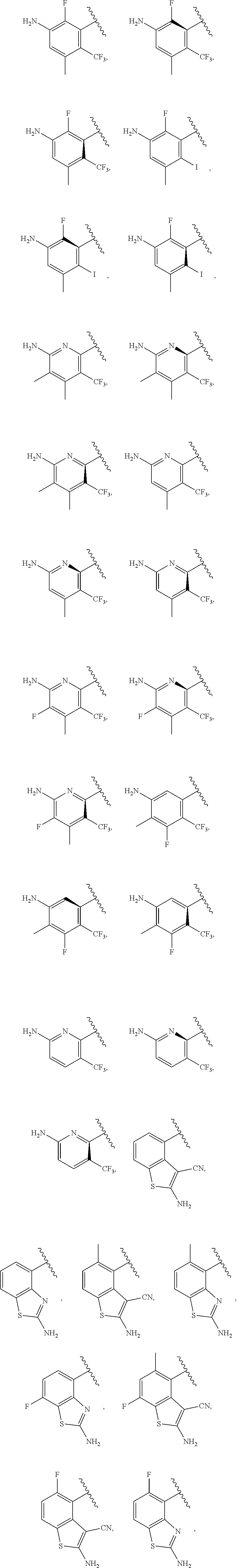

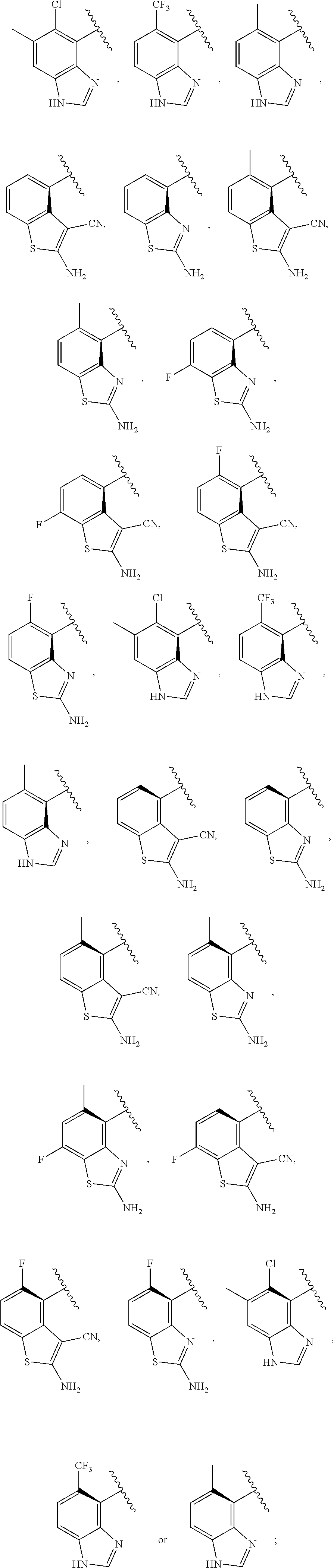

In some embodiments, R₄ is

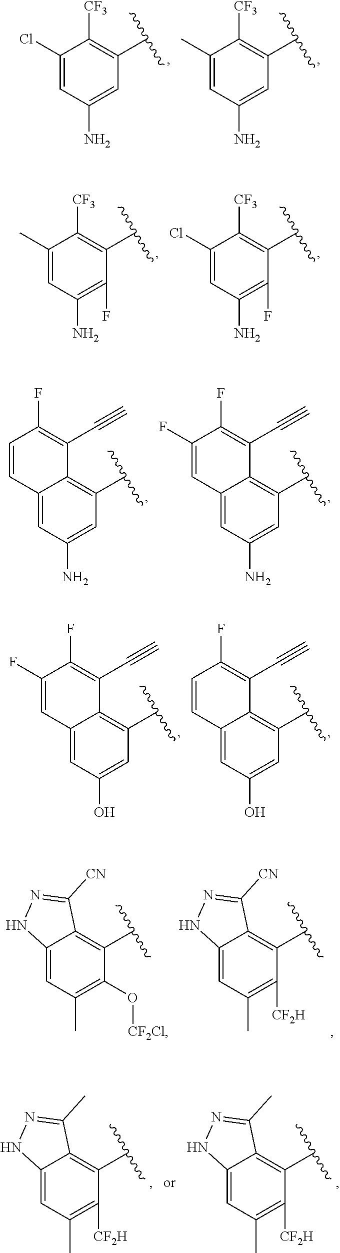

In some embodiments, R₄ is

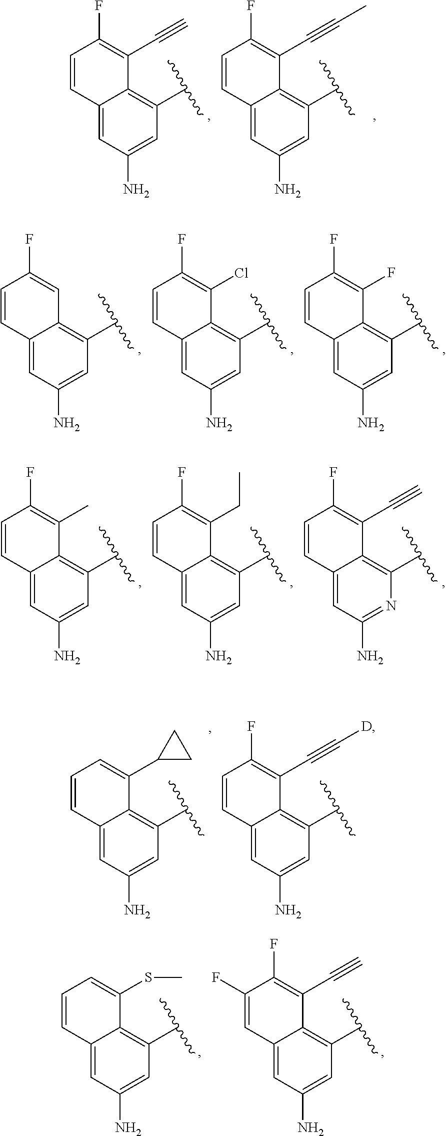

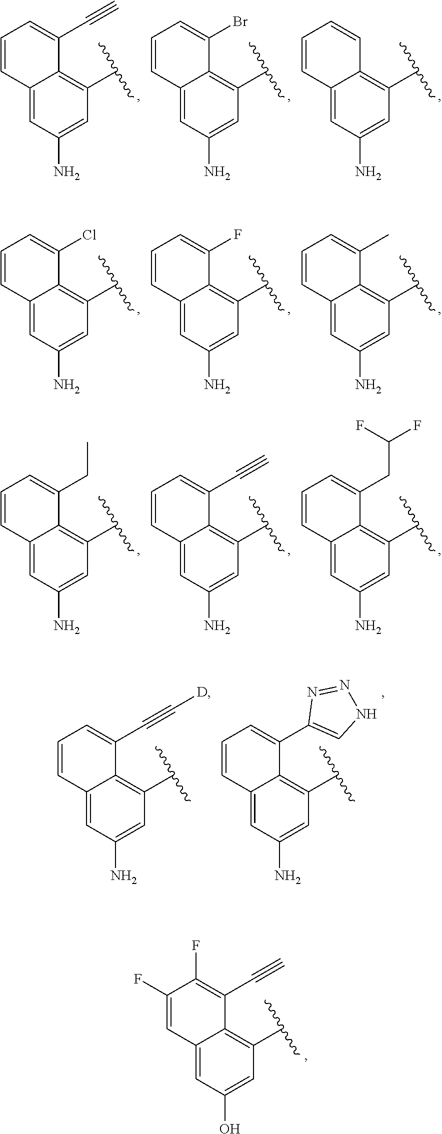

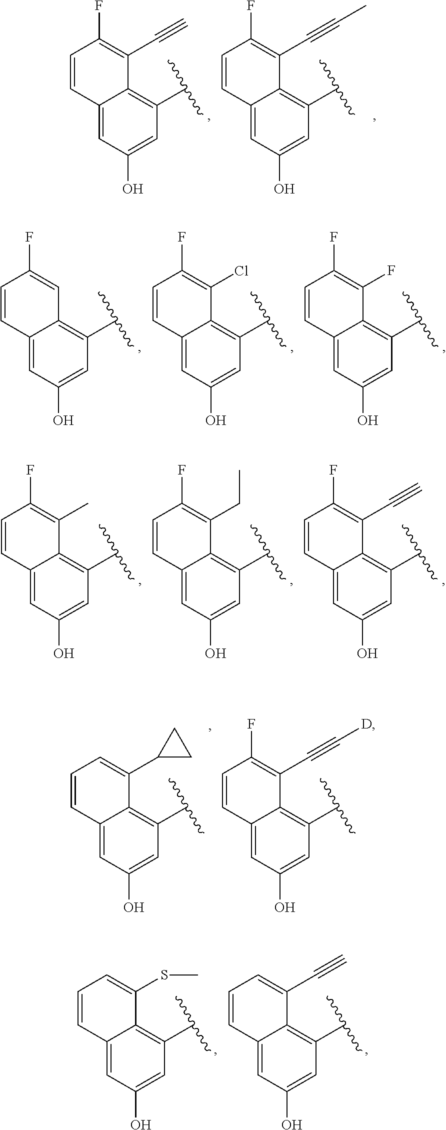

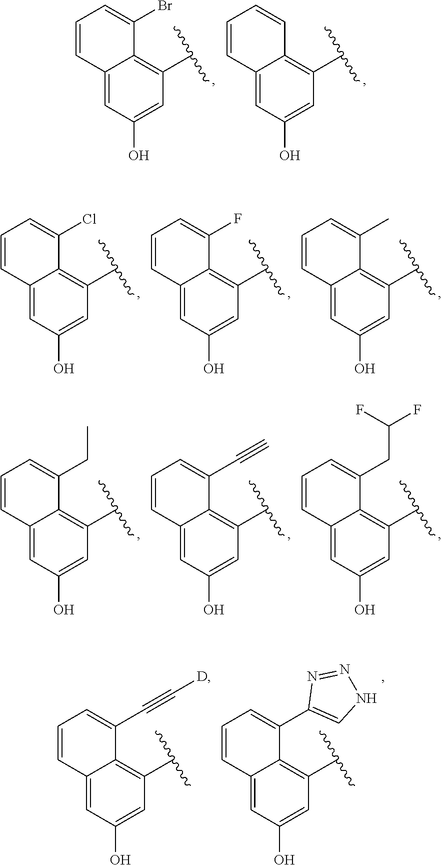

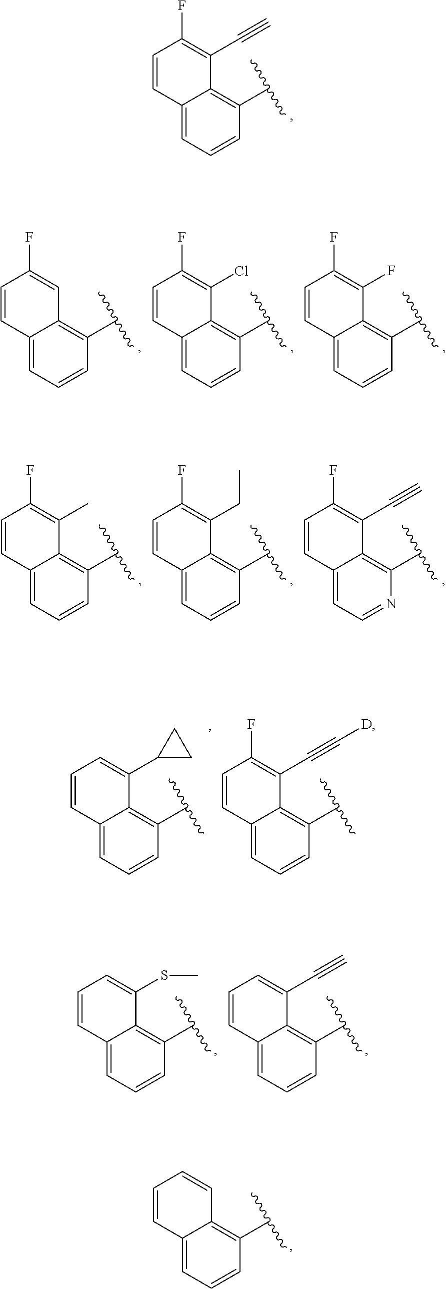

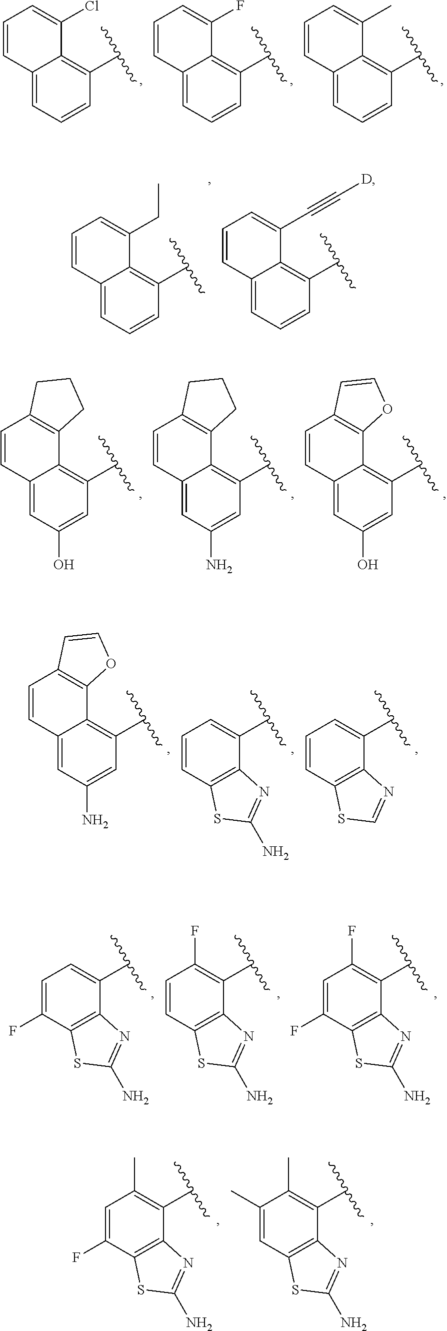

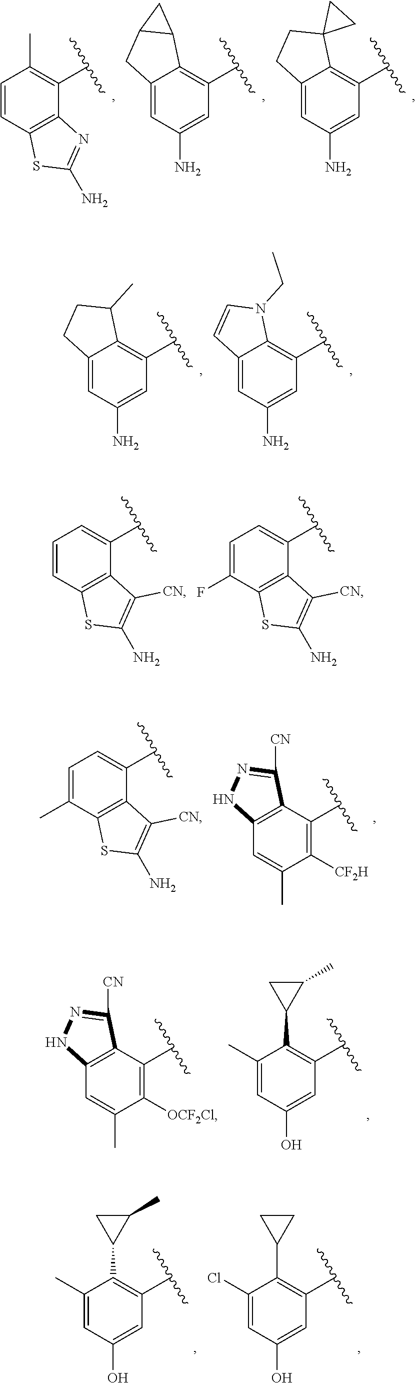

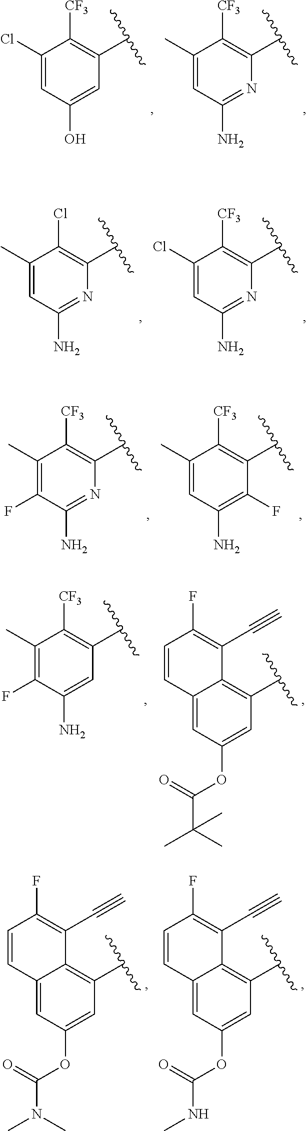

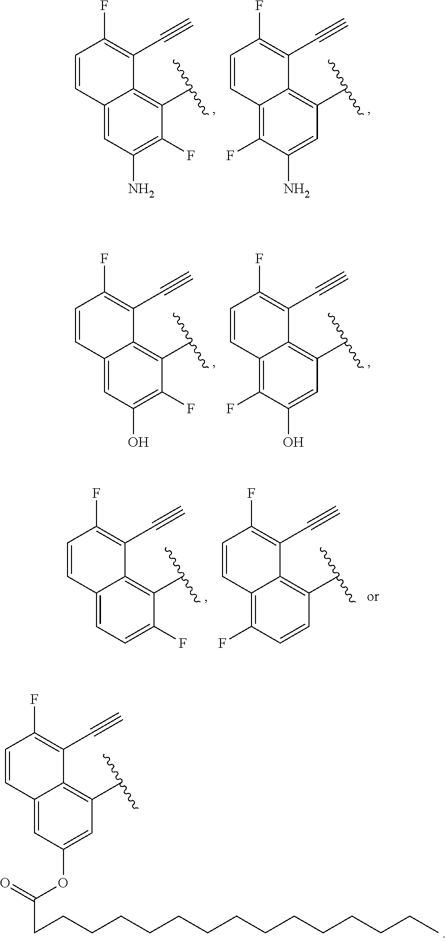

In some embodiments, R₄ is

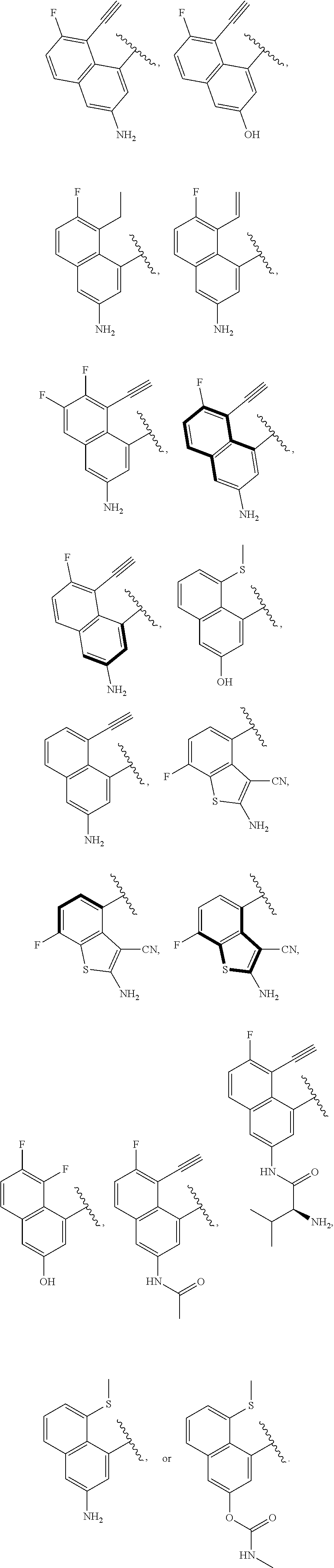

[64]. The compound of any one of [1] to [63], wherein, R₅ is halogen.

[65]. The compound of any one of [1] to [64], wherein, R₅ is —F.

In some embodiments, the compound is:

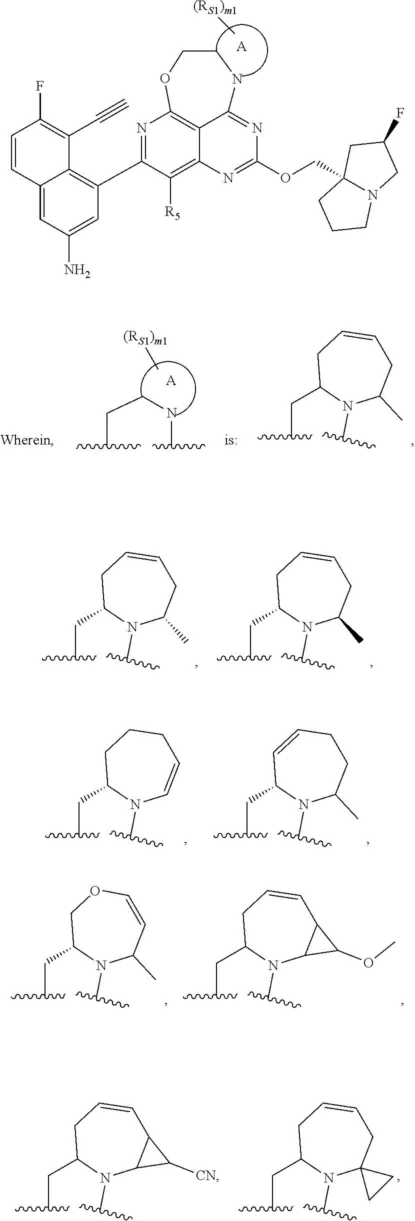

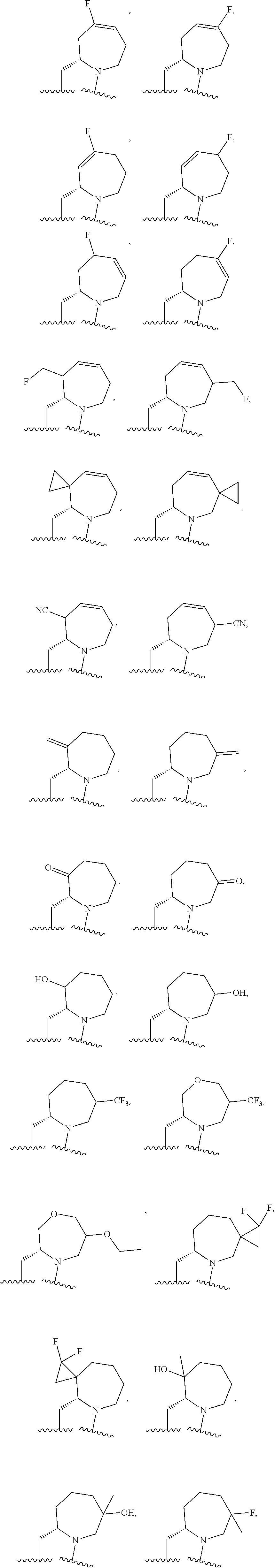

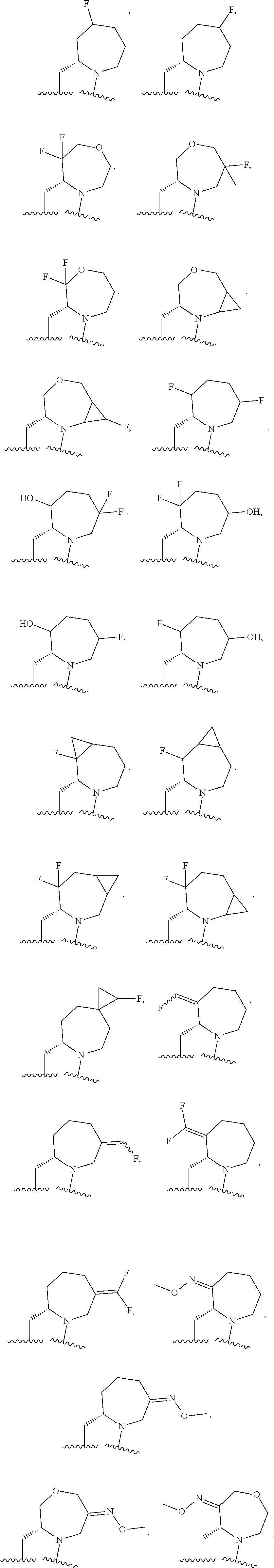

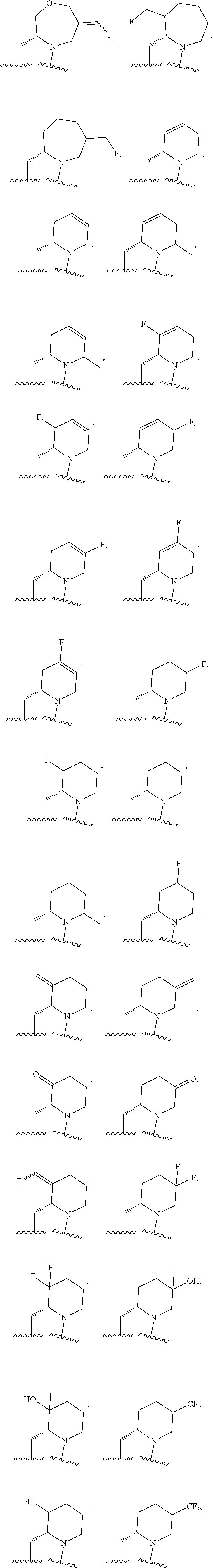

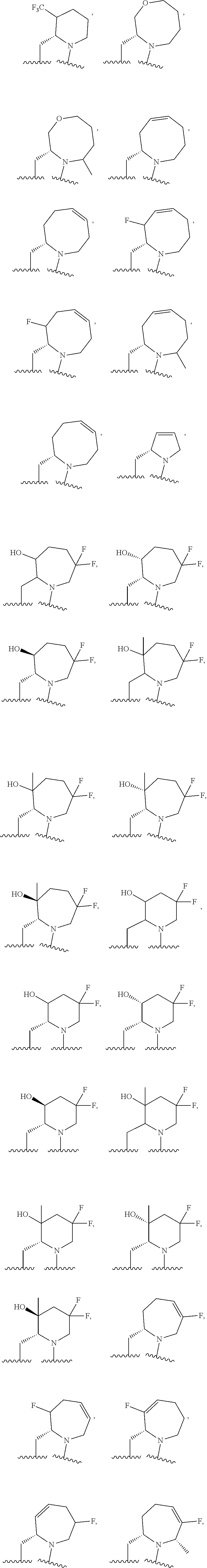

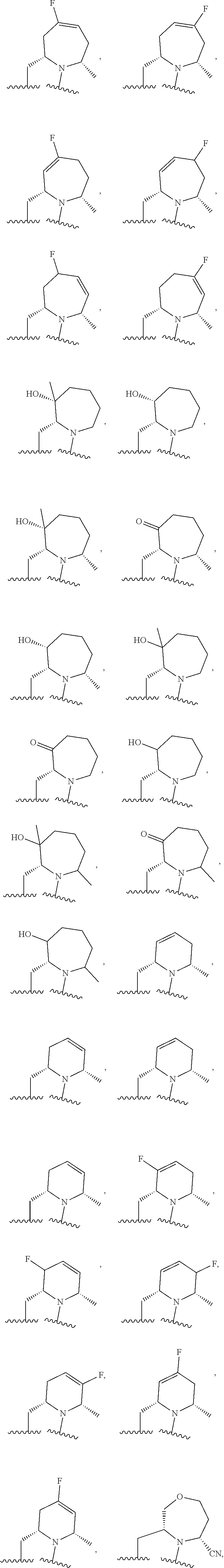

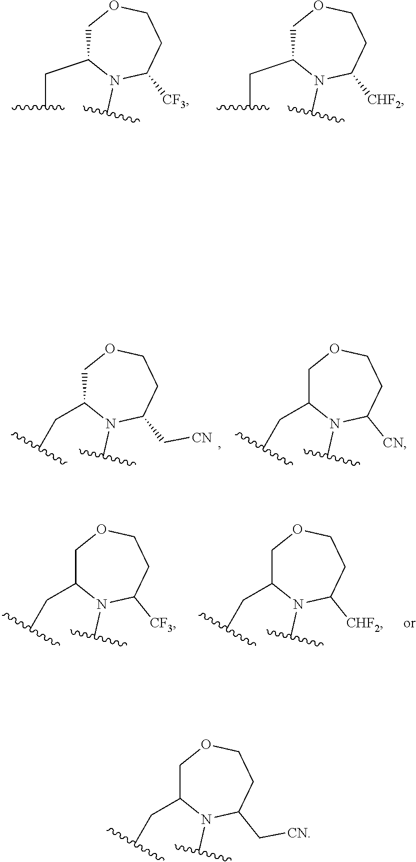

In some embodiments, the compound is:

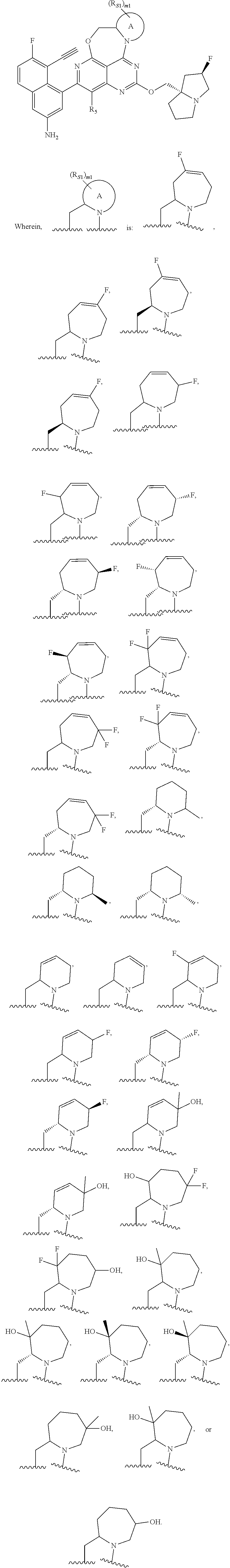

In some embodiments, the compound is:

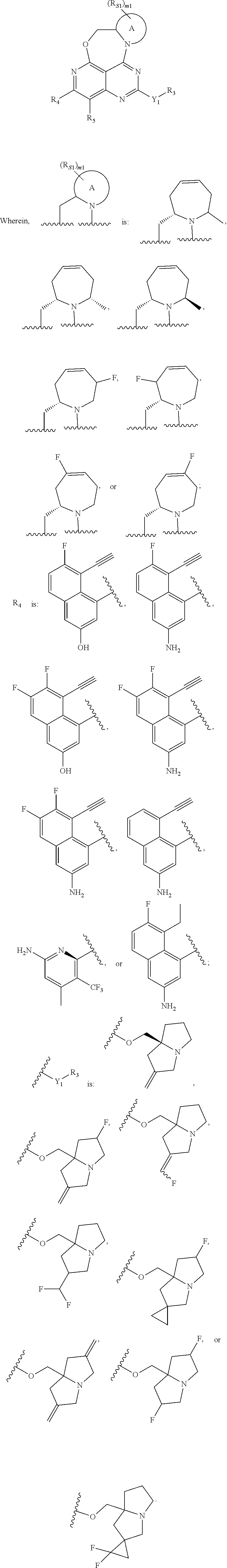

In some embodiments, the compound is any one of the following formulas:

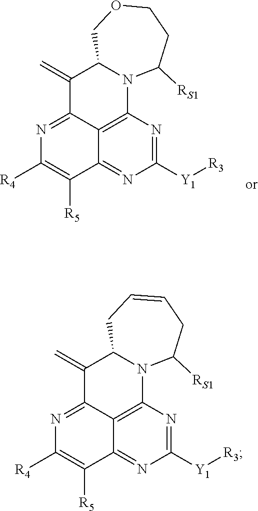

R₅ is —F, —Cl, —CH₃ or —OCH₃;
R_{S1} is —H, —CH₃ or —CD₃;
—Y₁—R₃ is:

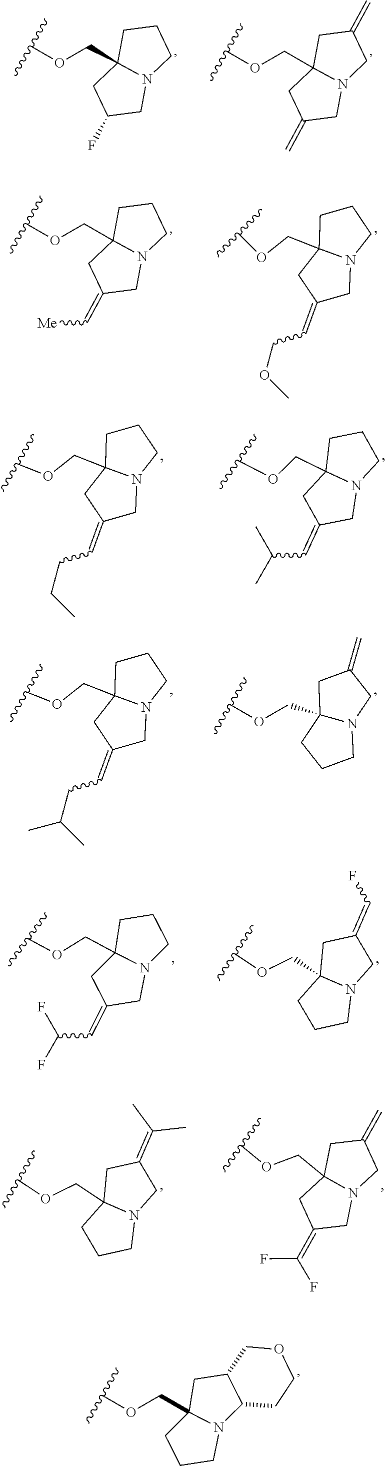

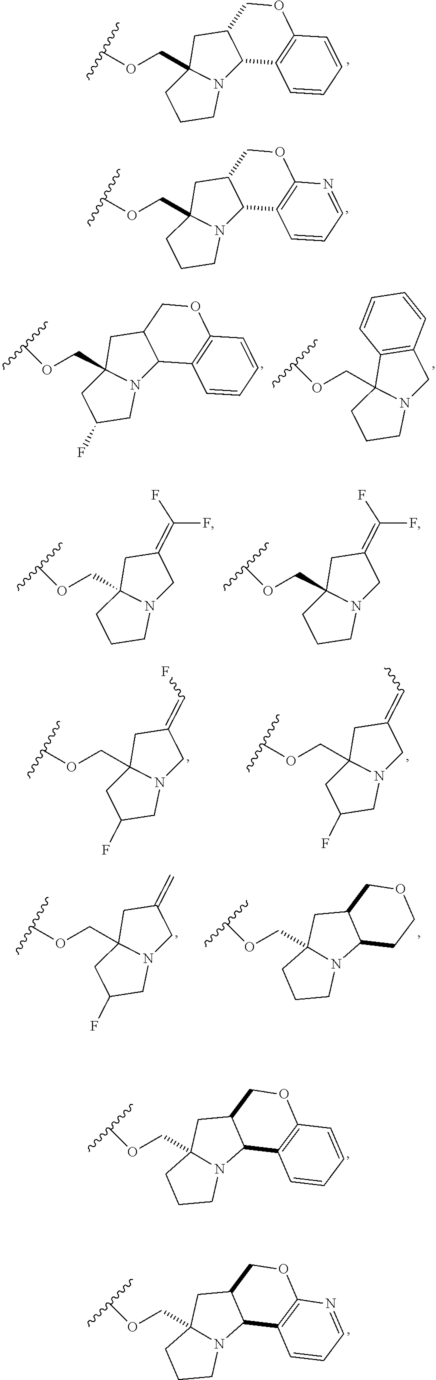

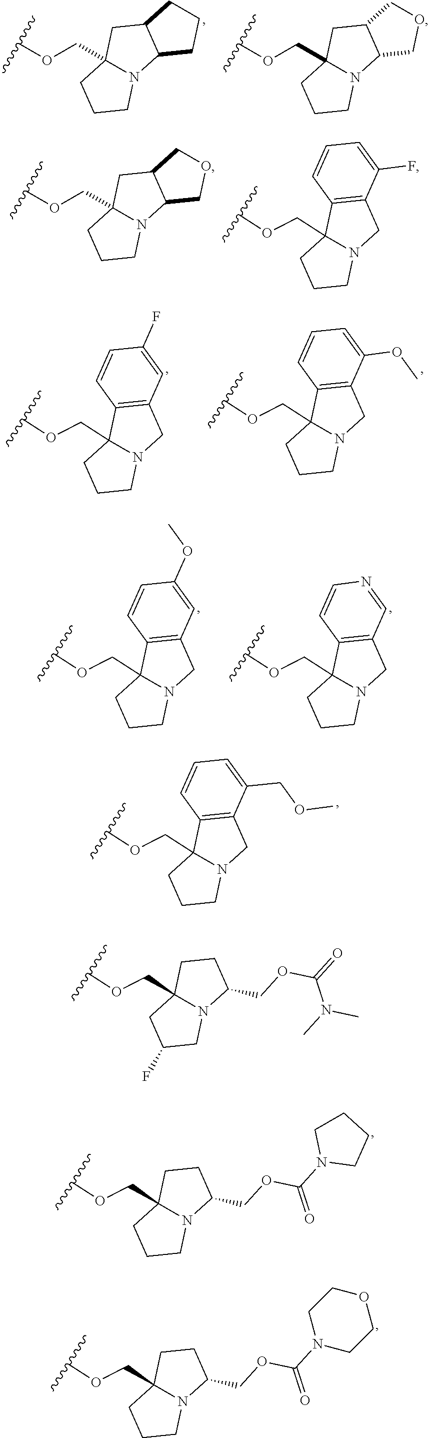

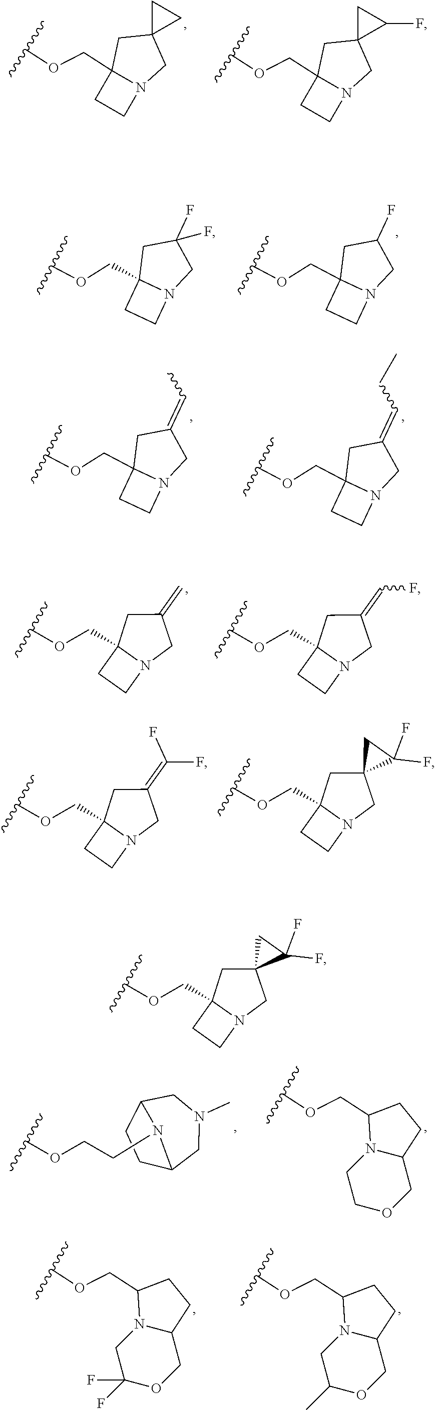

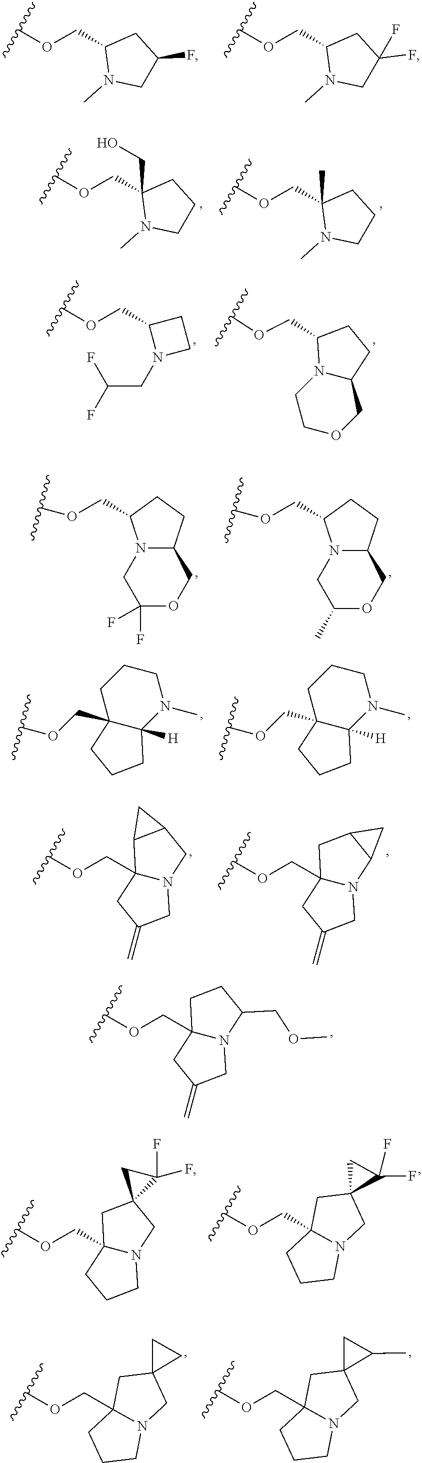

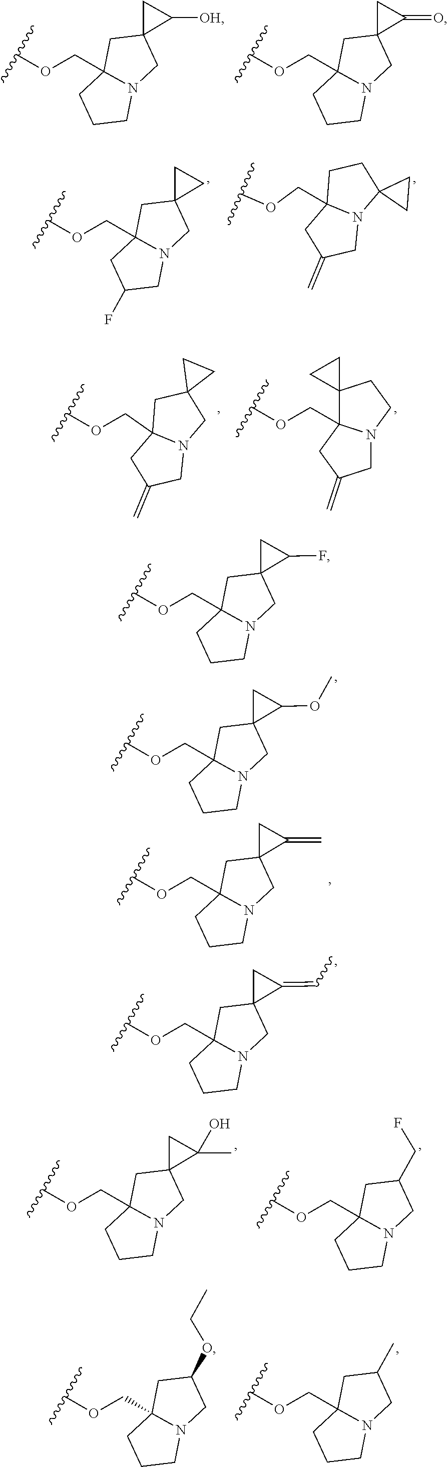

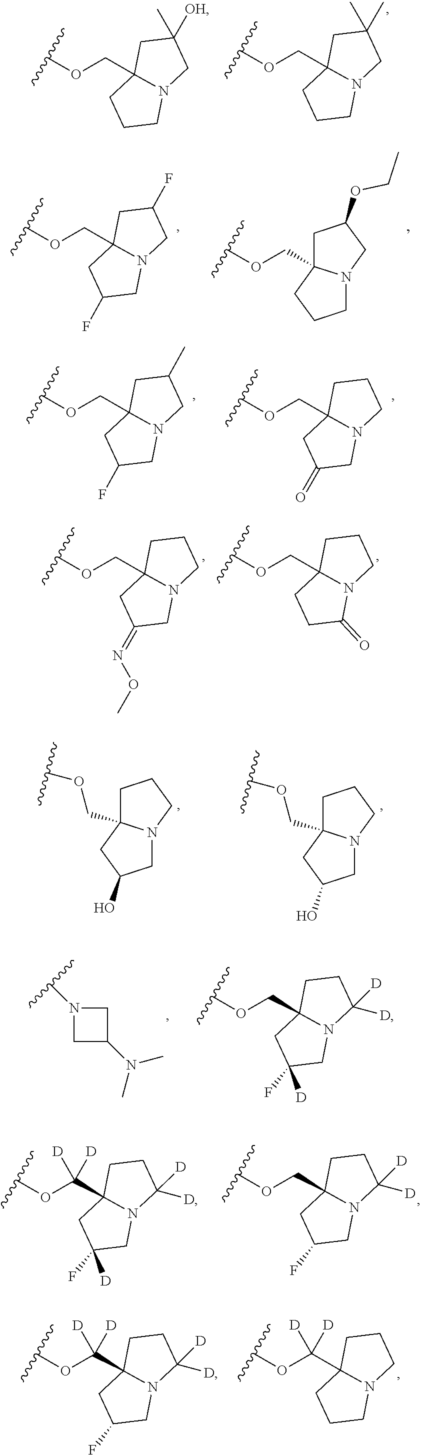

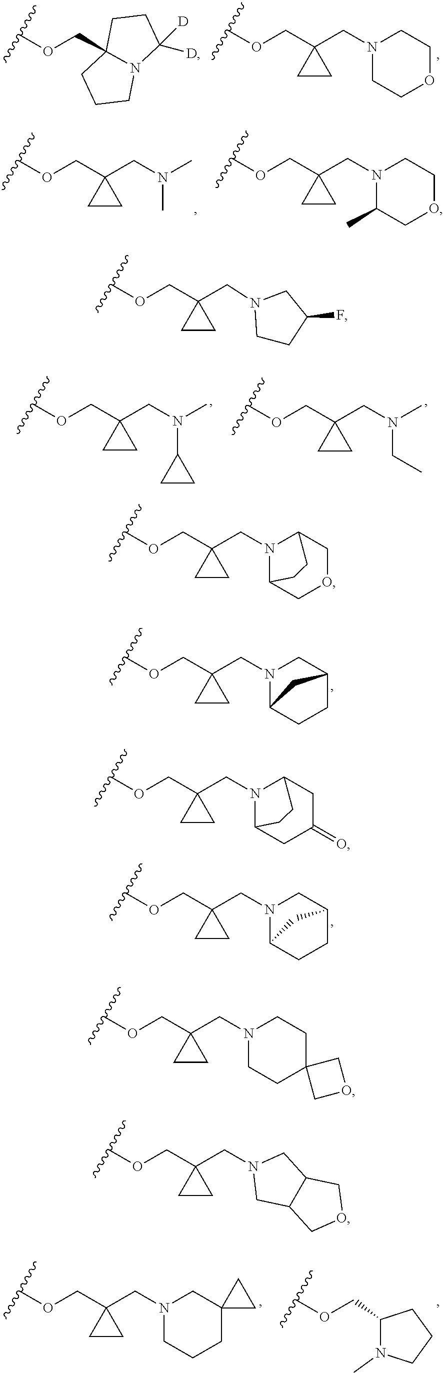

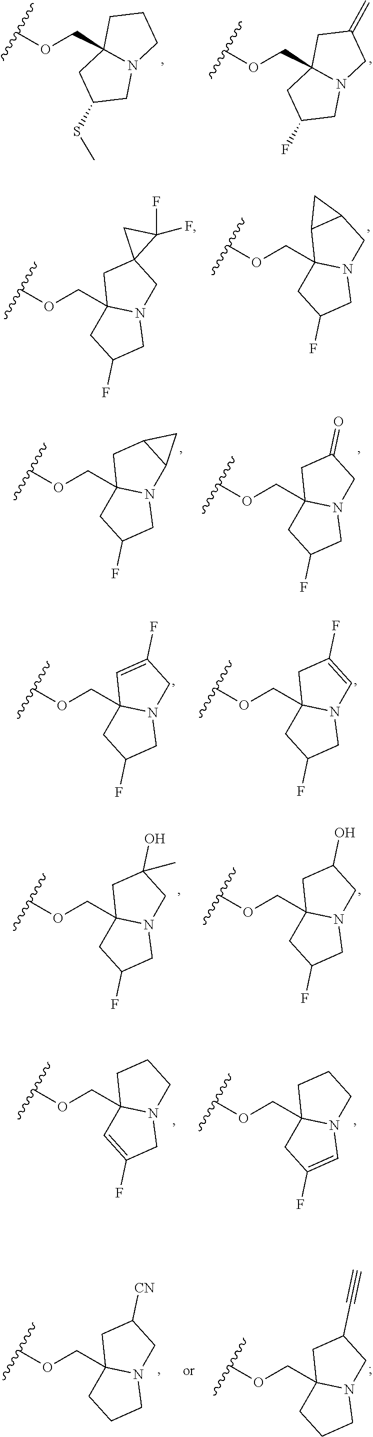

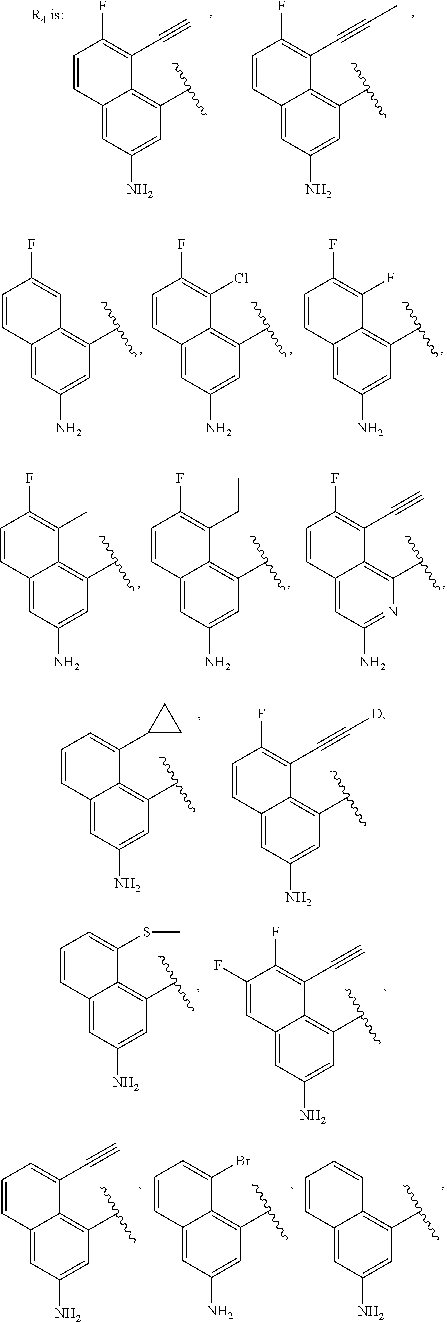

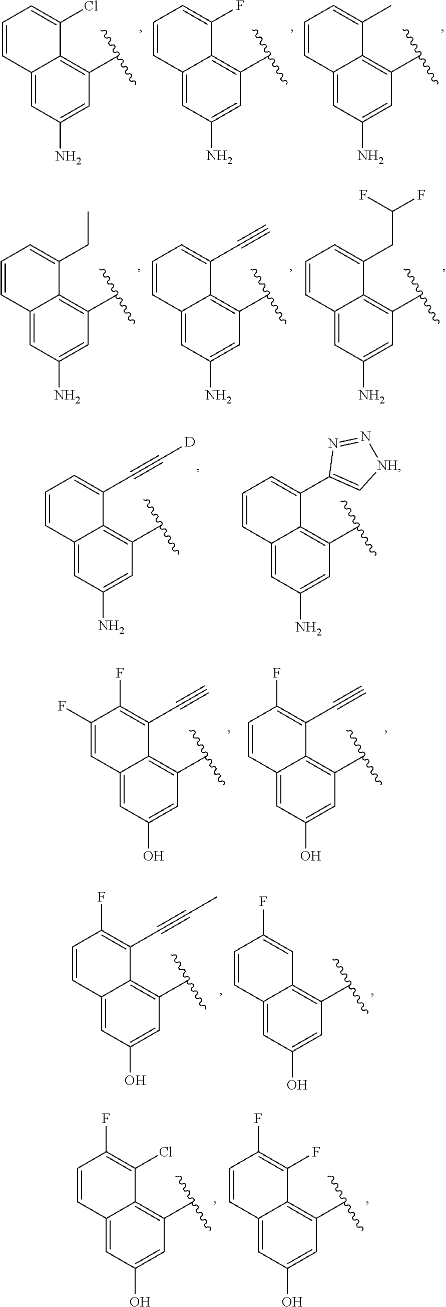

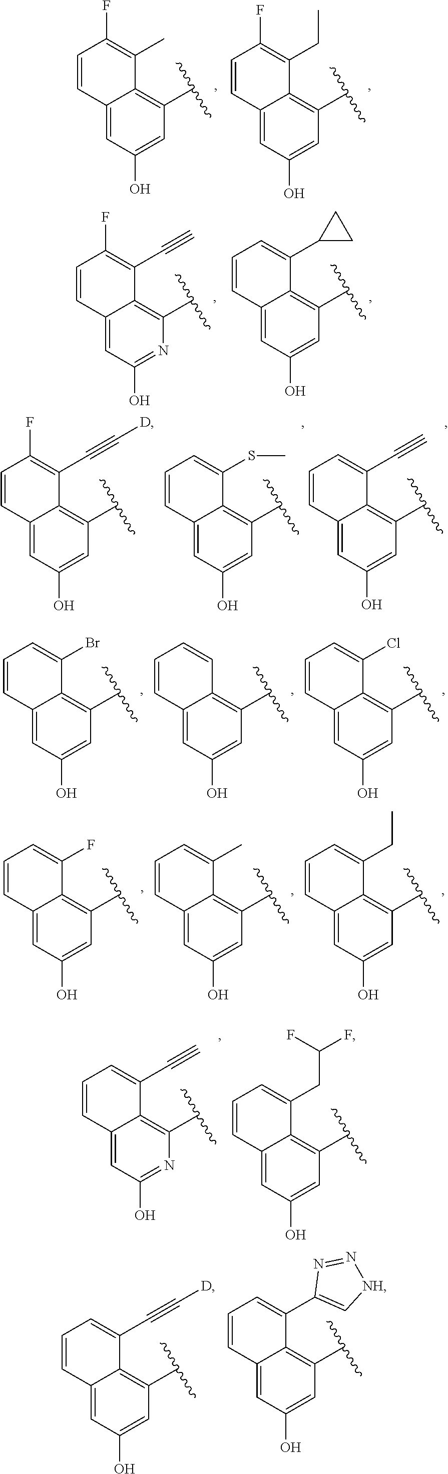

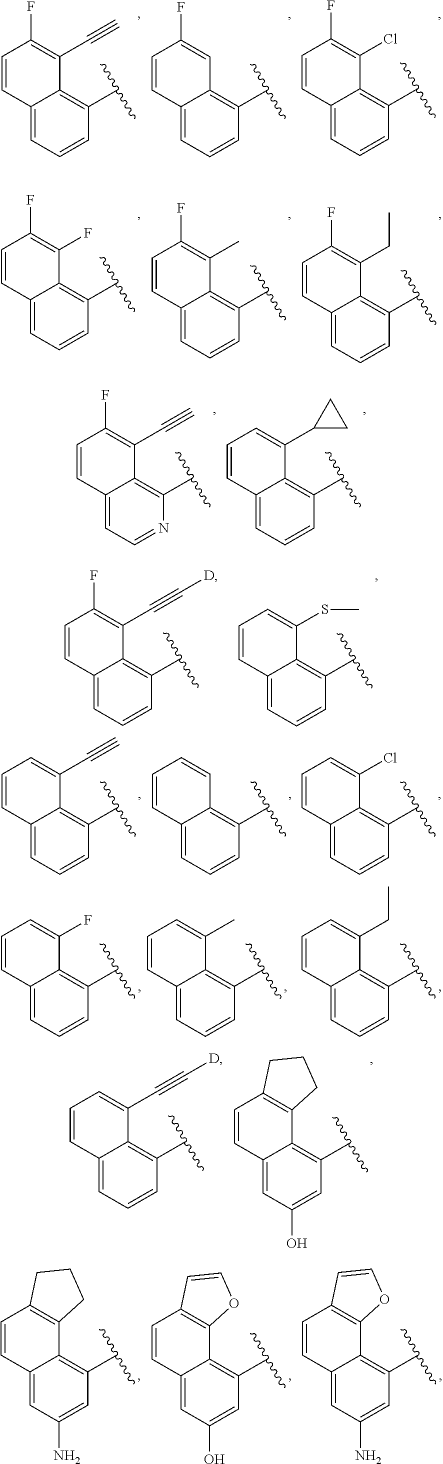

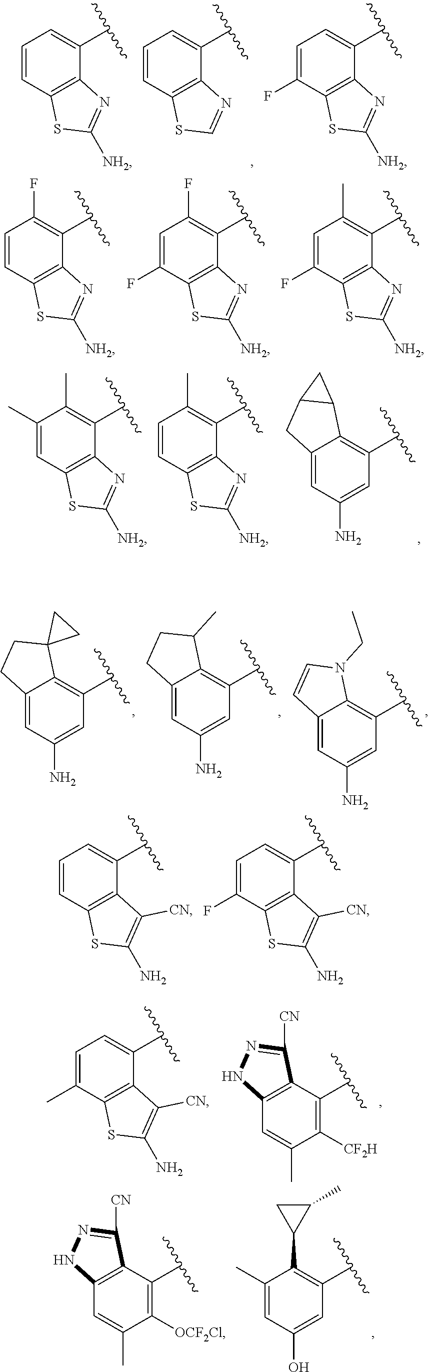

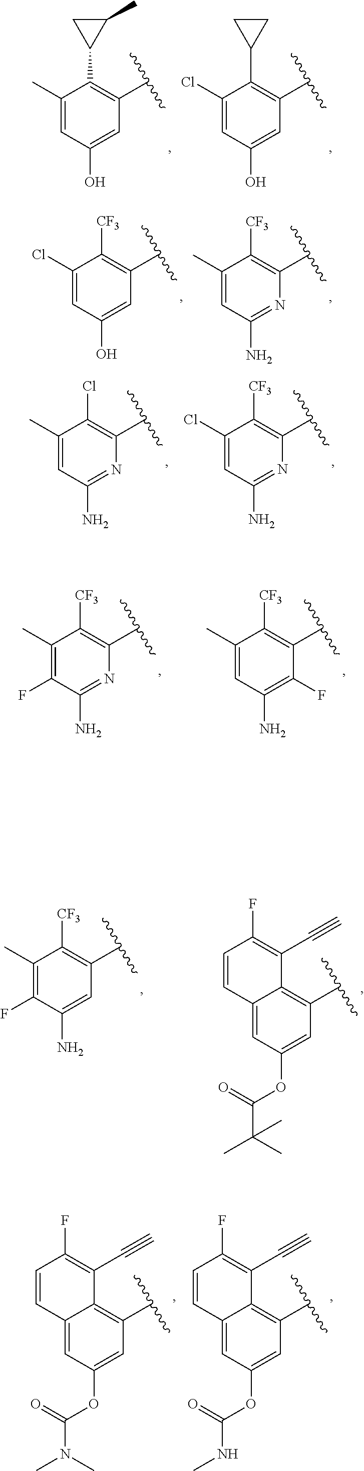

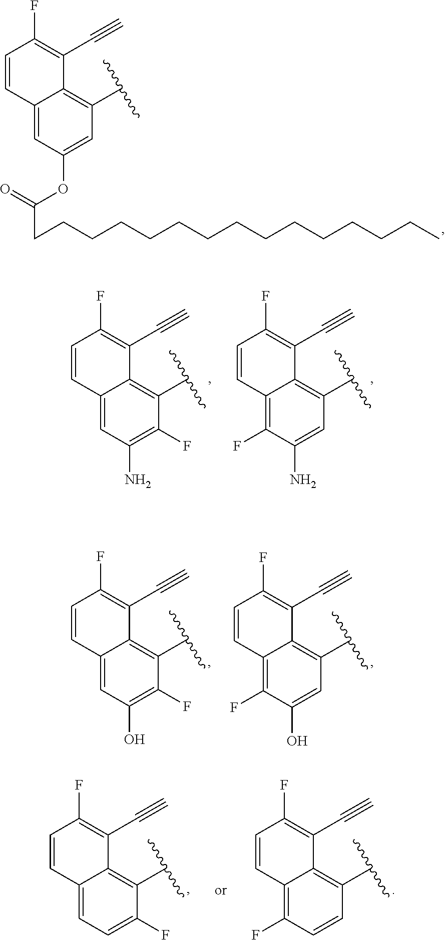

In some embodiments, the compound is any one of the following formulas:

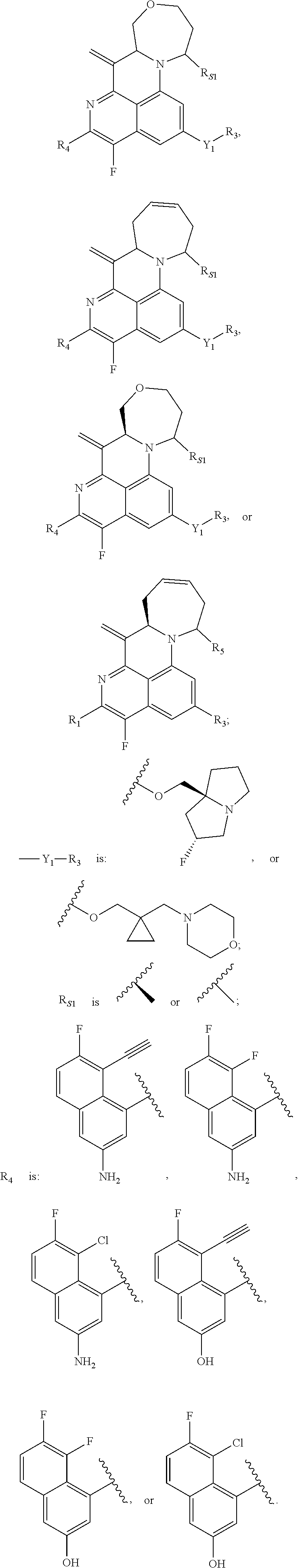

In some embodiments, the compound is any one of the following formulas:

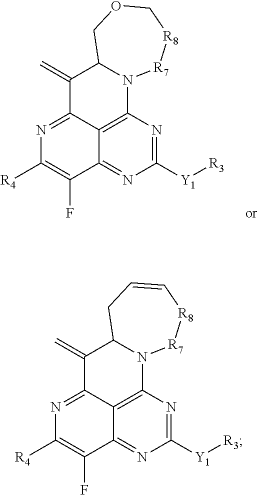

Wherein:

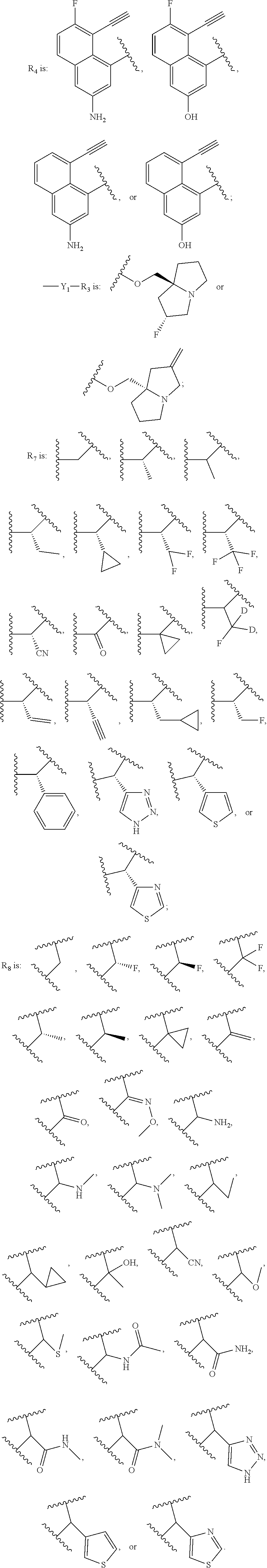

in some embodiments, the compound is any one of the following formulas:

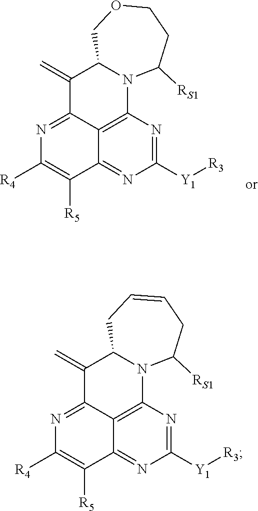

Wherein:
R₅ is —F, —Cl, —CH₃ or —OCH₃;
R_{S1} is —H,

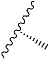

or —CD₃;
—Y₁—R₃ is:

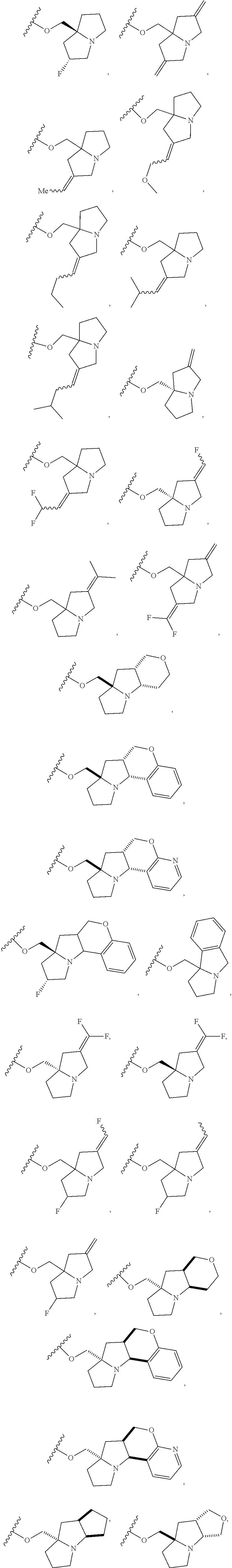

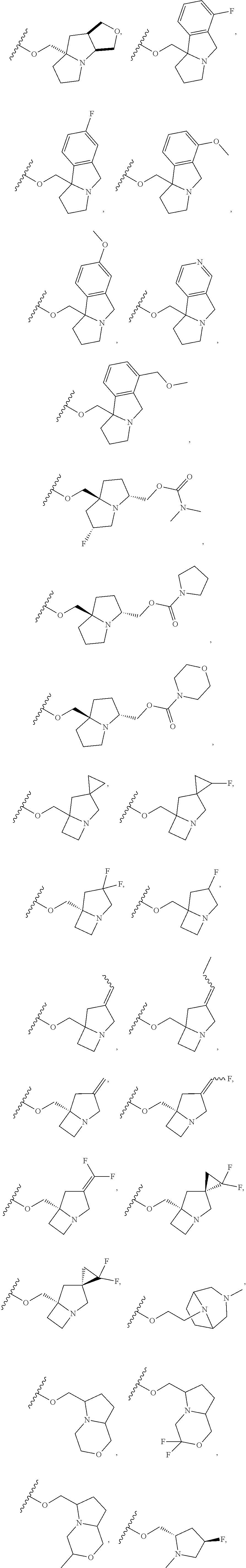

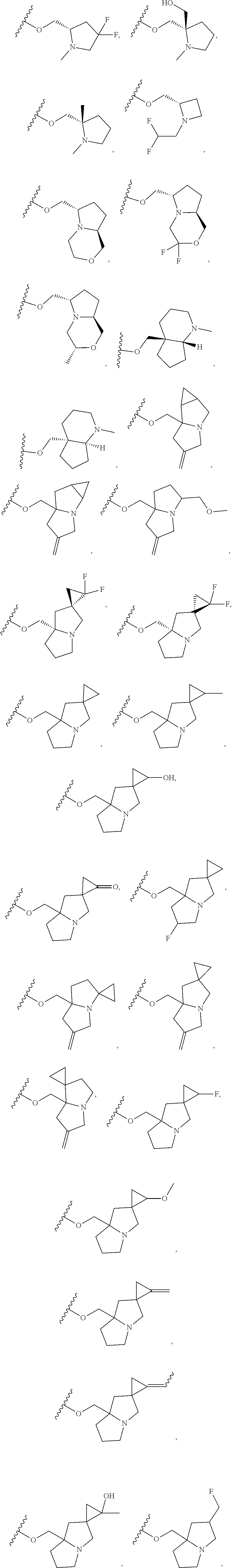

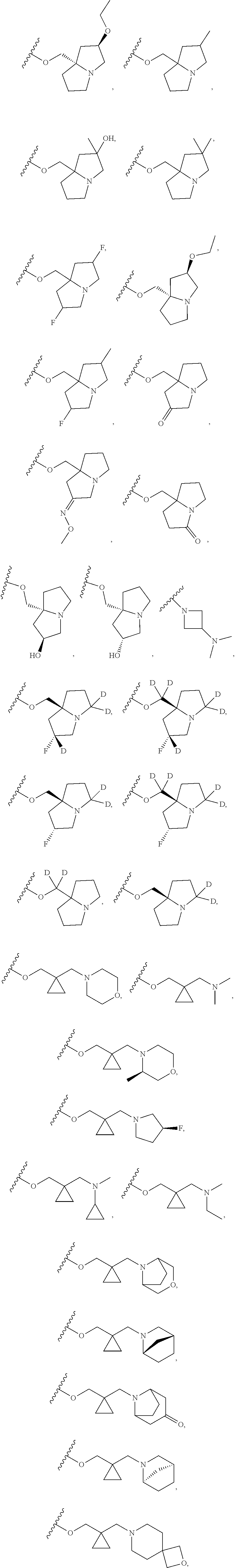

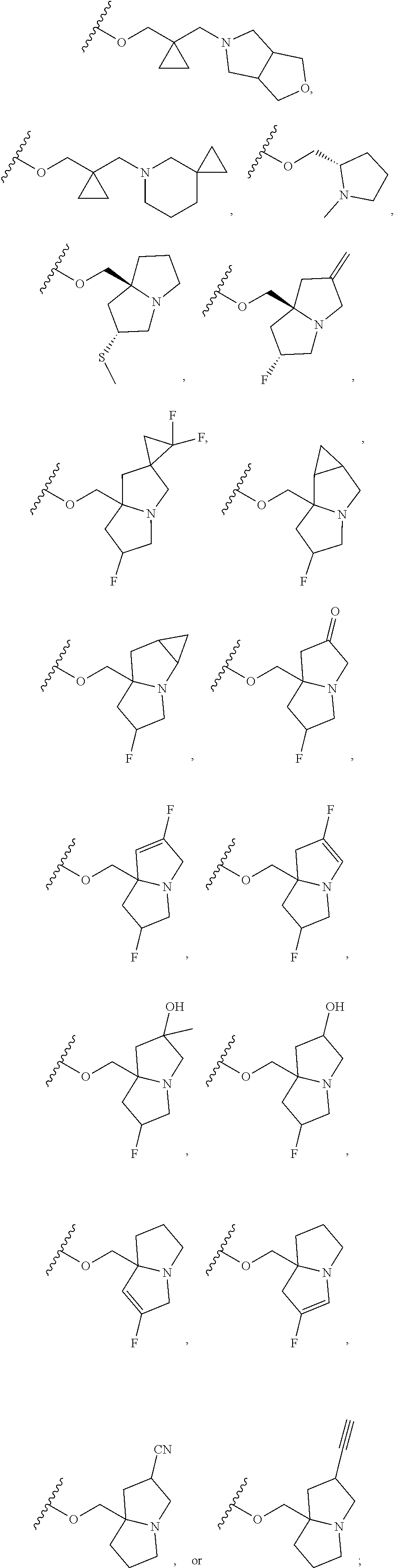

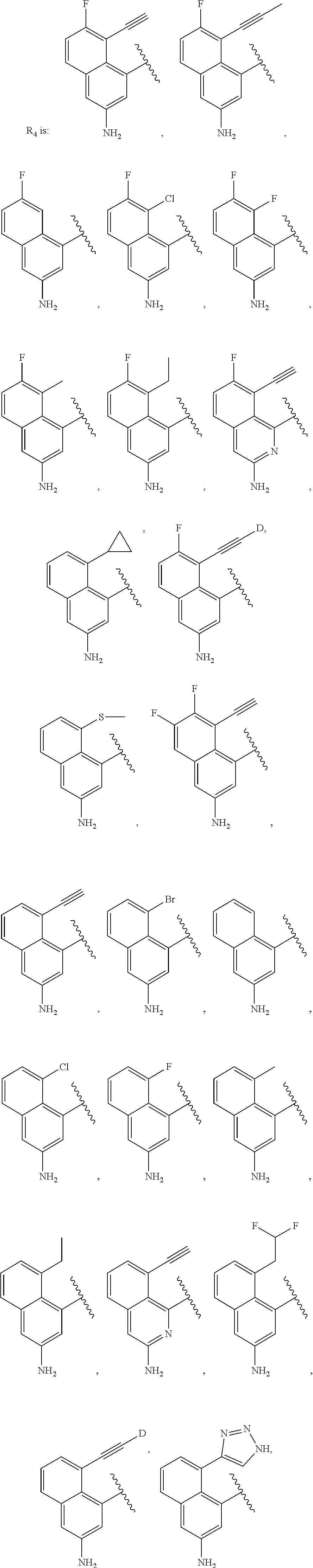

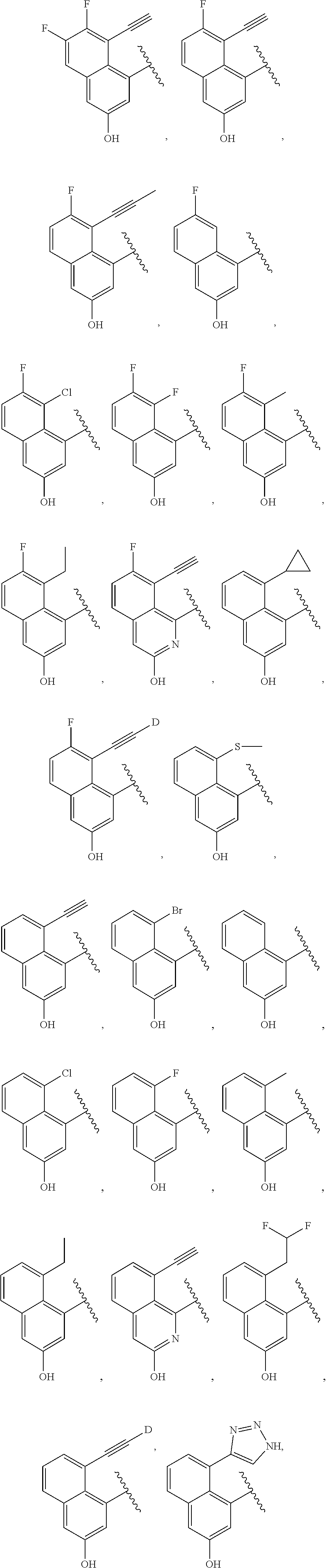

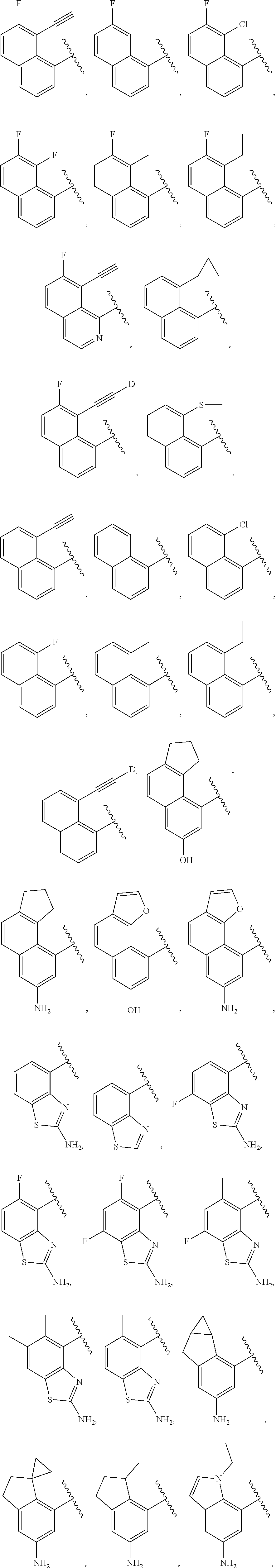

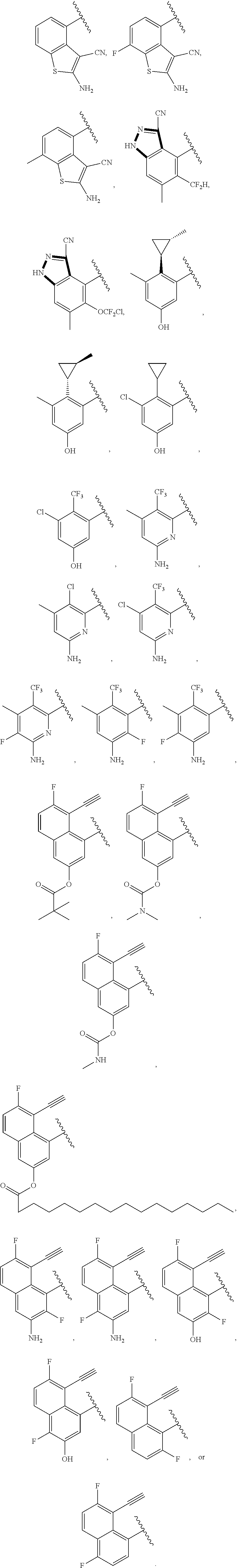

In some embodiments, the compound is any one of the following formulas:

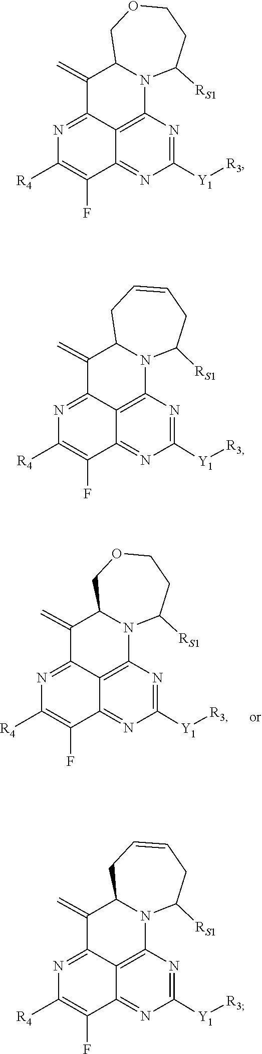

Wherein:
—Y₁—R₃ is:

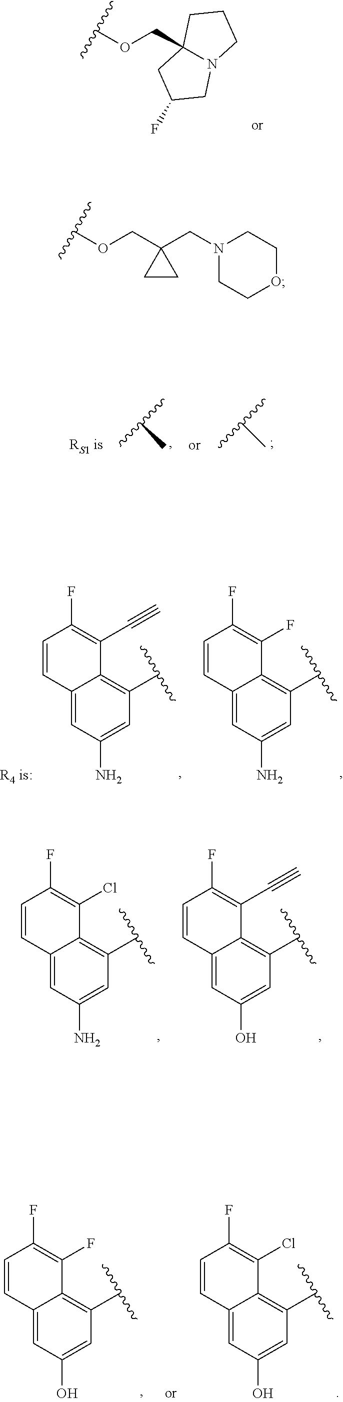

In some embodiments, the compound is any one of the following formulas:

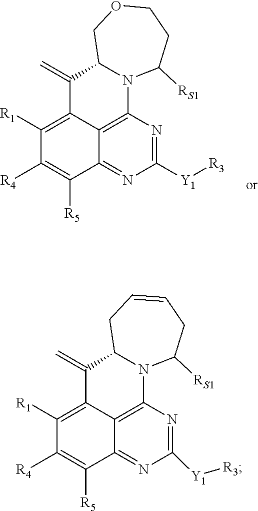

R₅ is —F, —Cl or —CH₃;
R_{S1} is

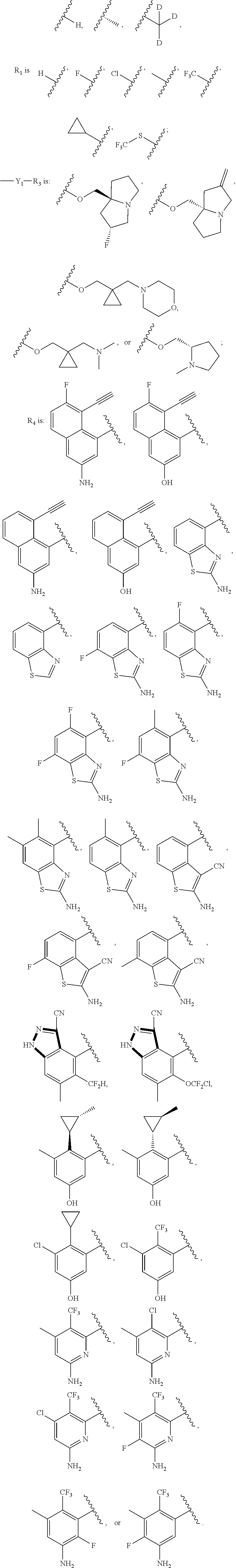

In some embodiments, the compound is any one of the following formulas:

In some embodiments, the compound is any one of the following formulas:

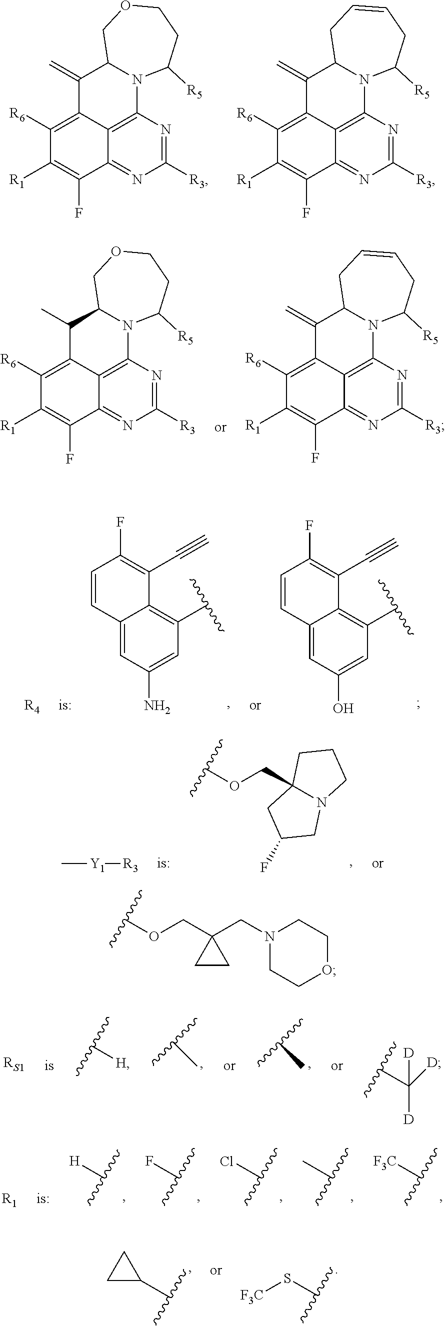

Wherein:
R₅ is:

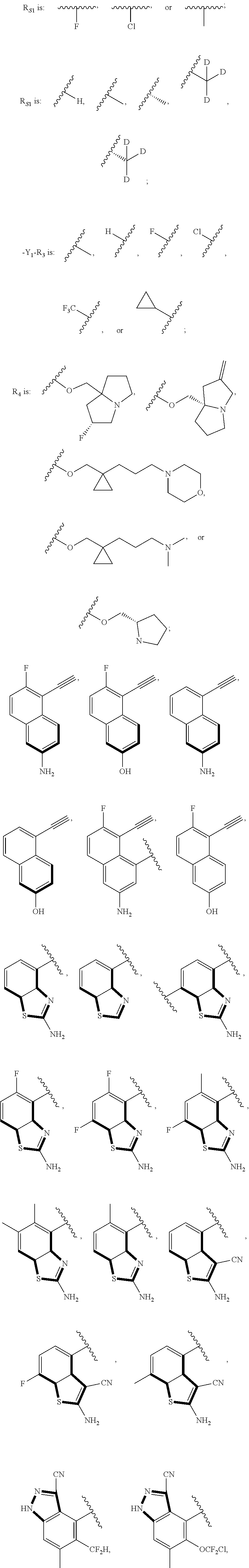

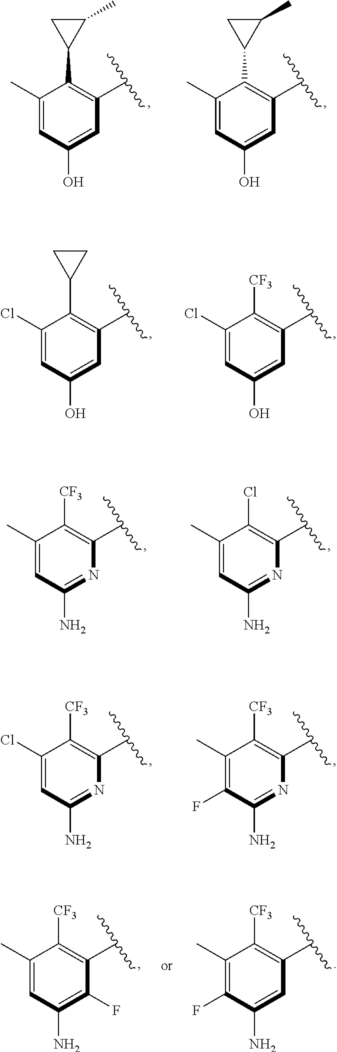

In some embodiments, the compound is any one of the following formulas:

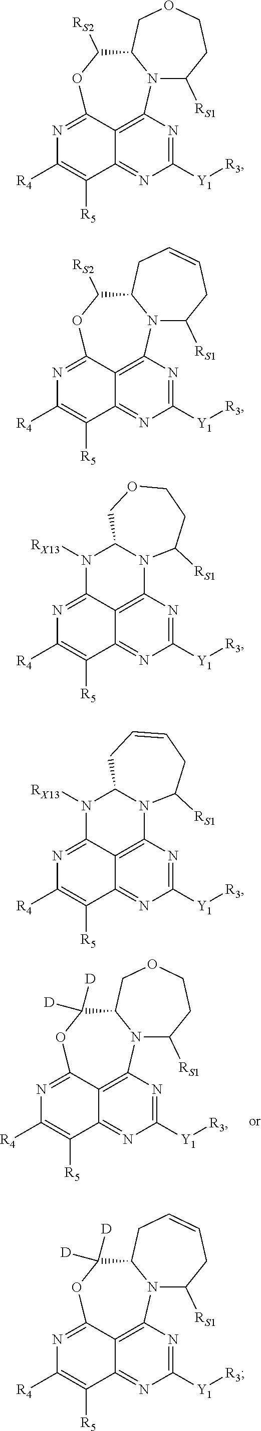

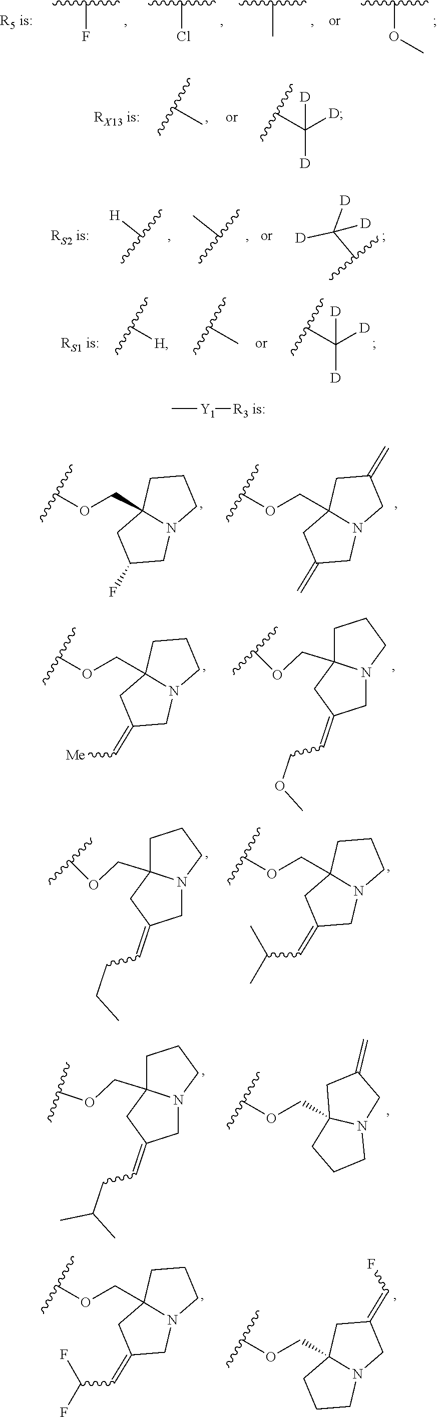

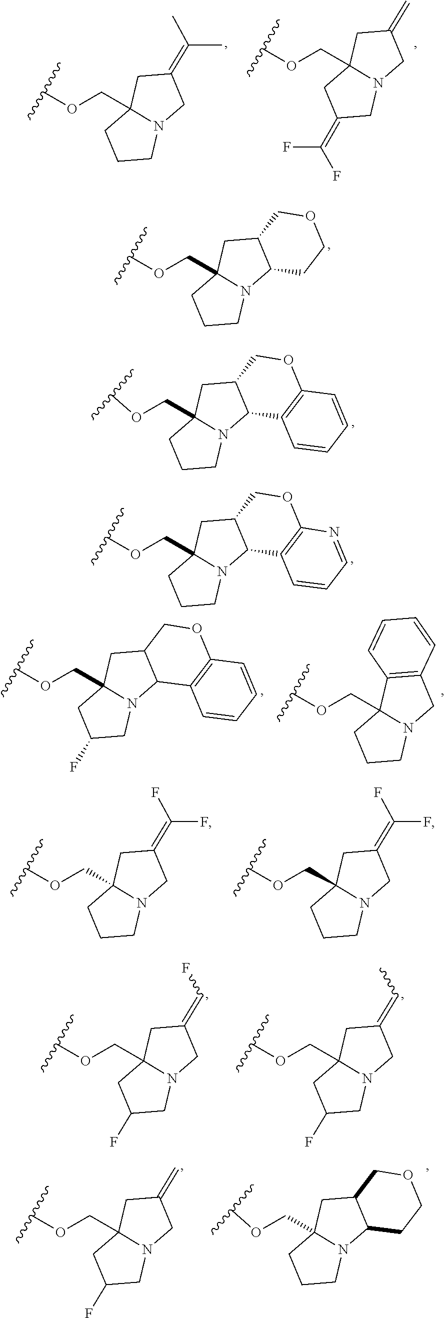

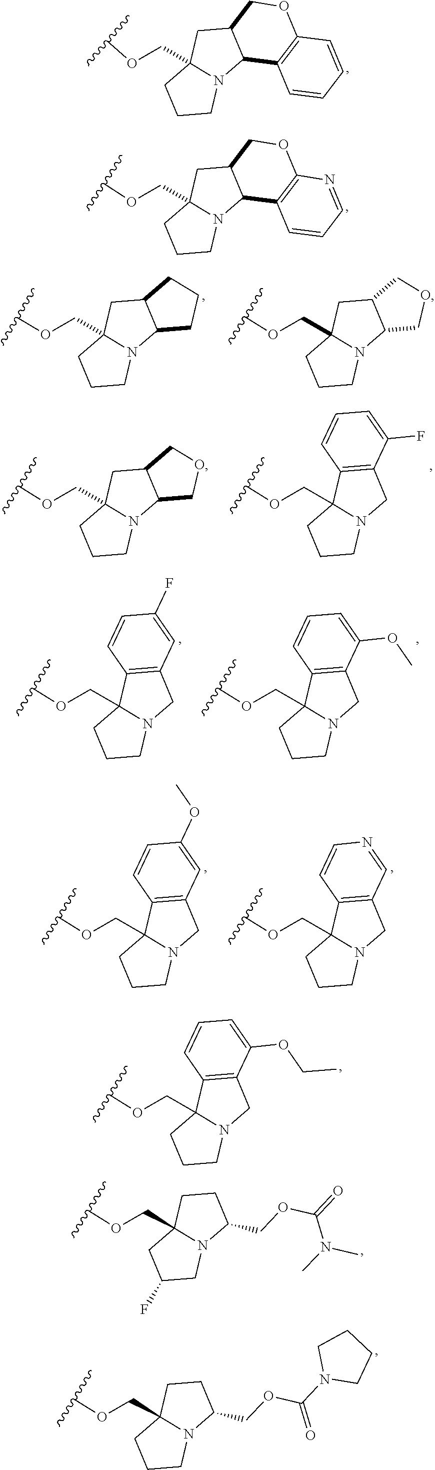

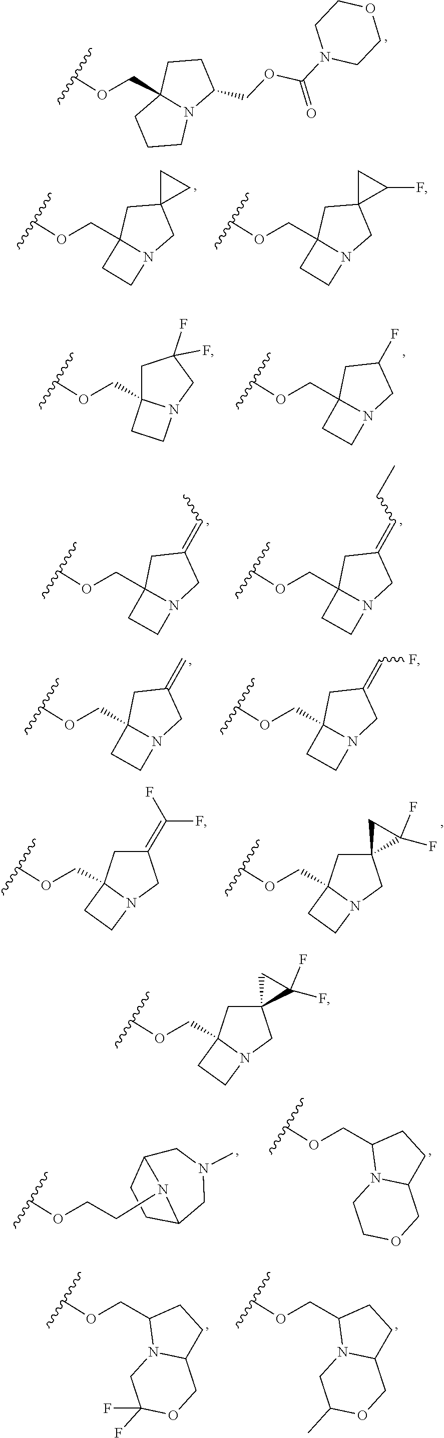

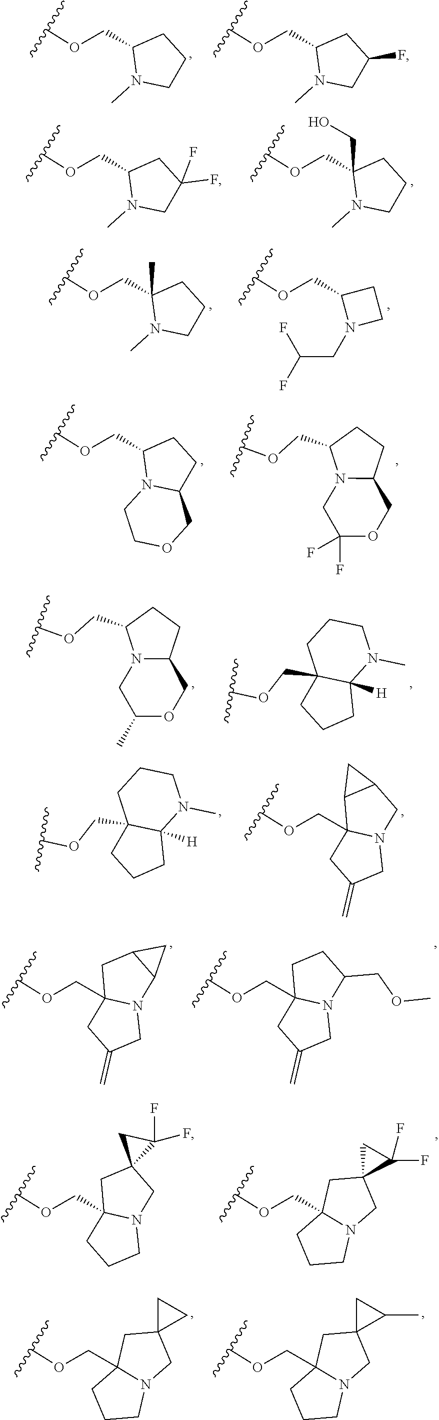

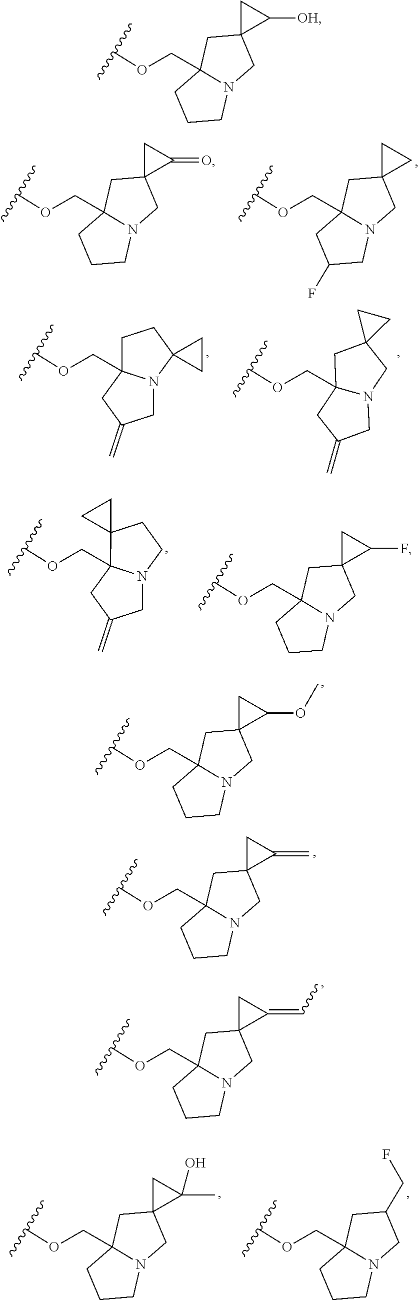

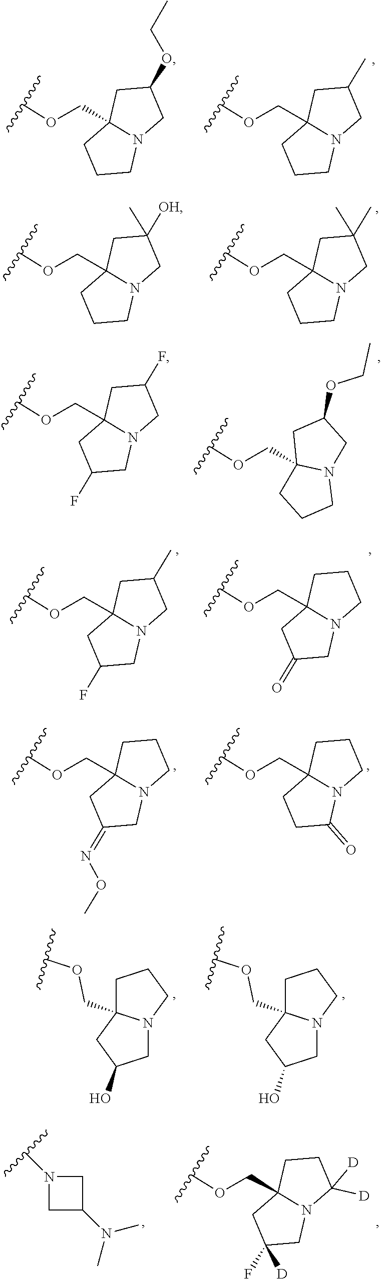

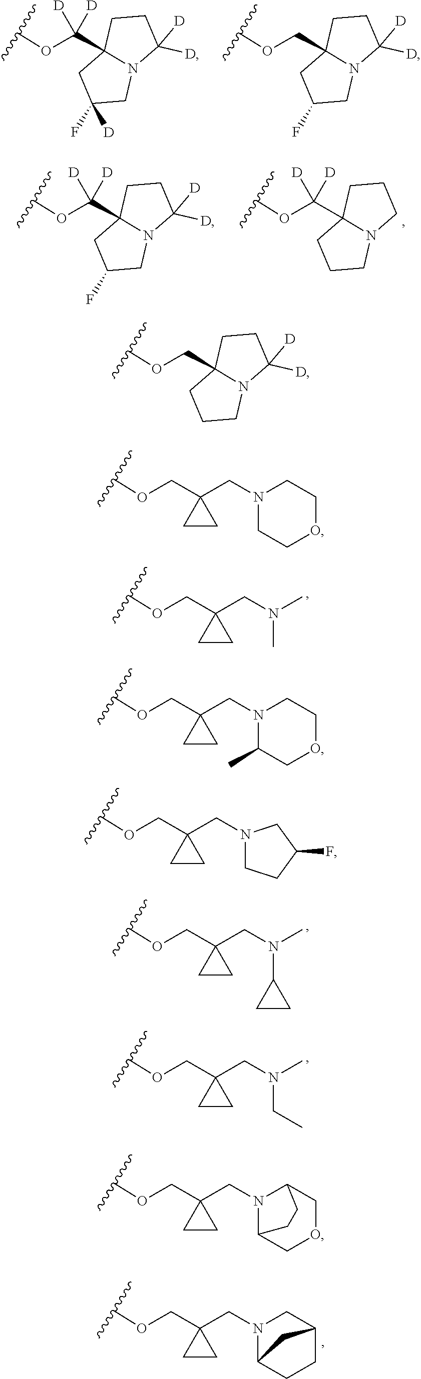

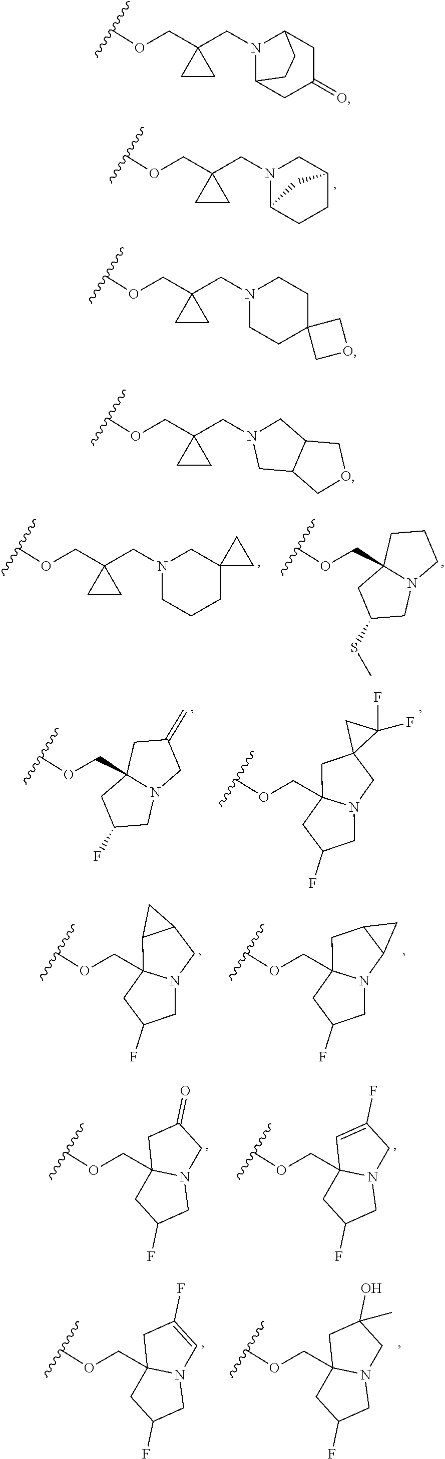

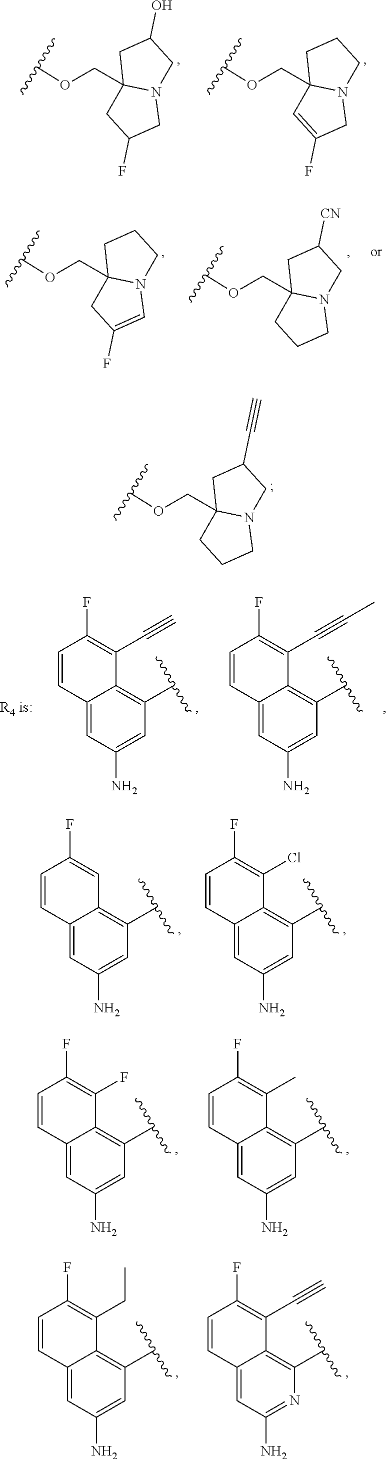

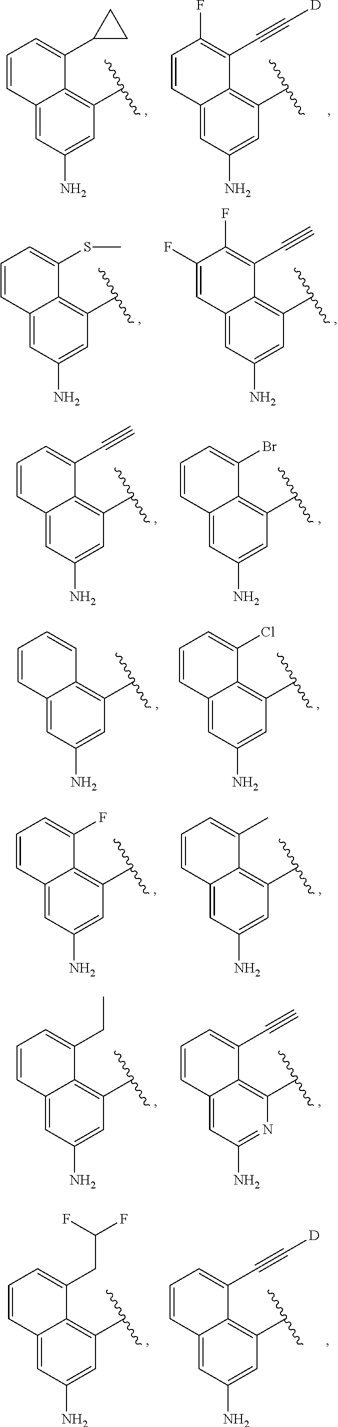

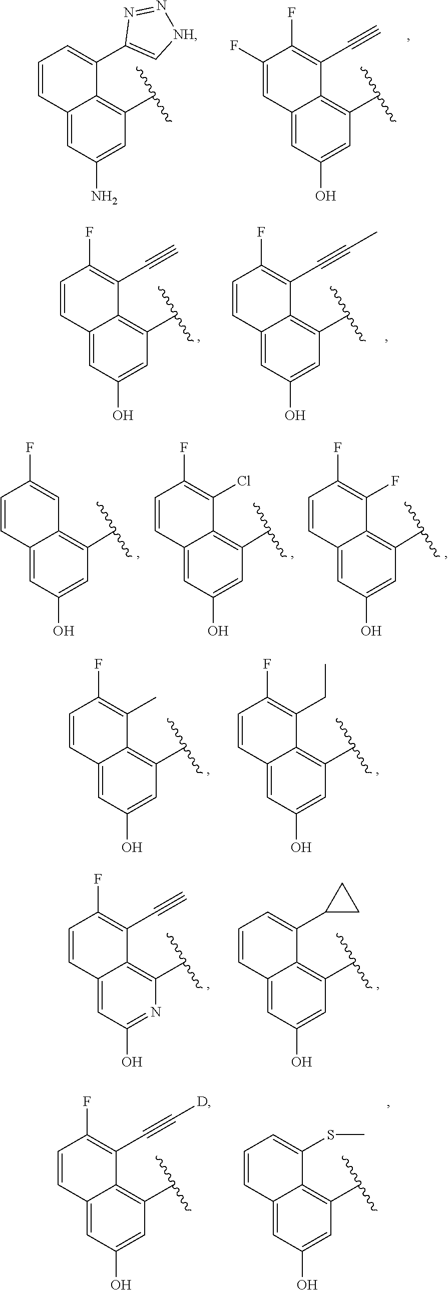

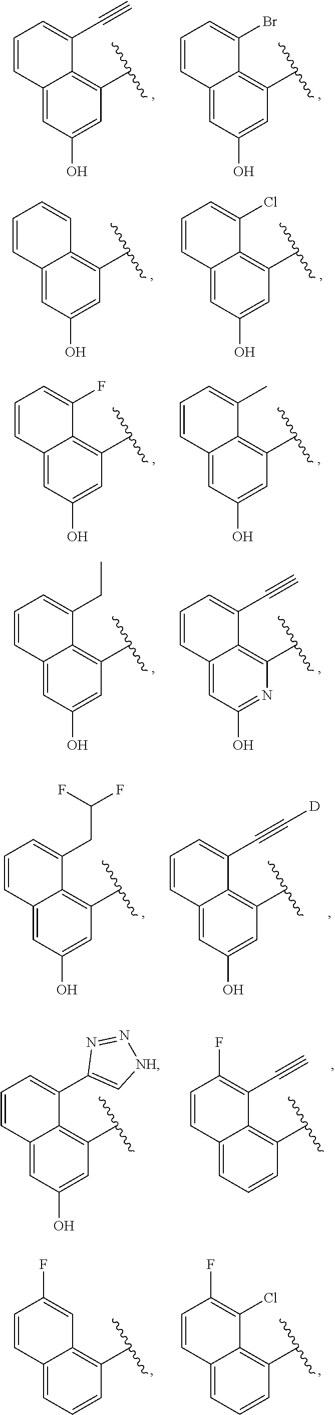

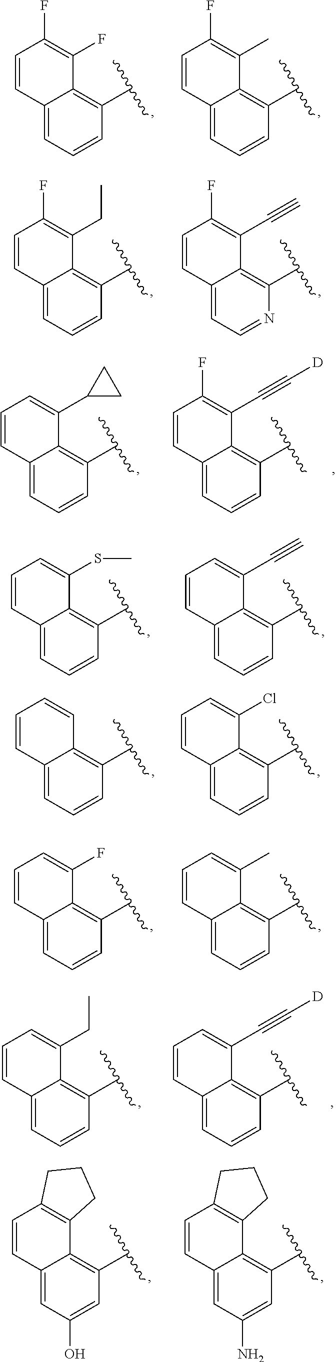

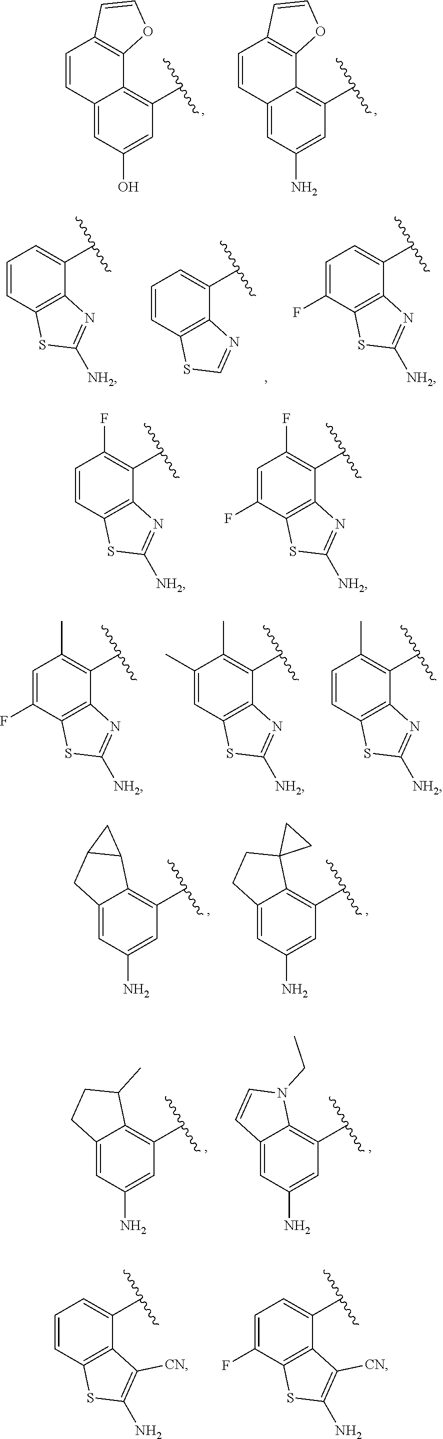

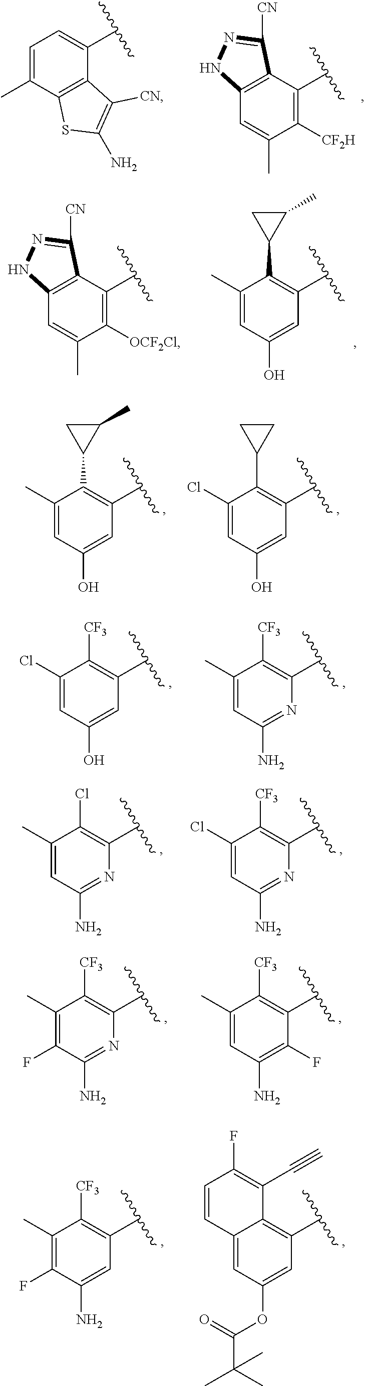

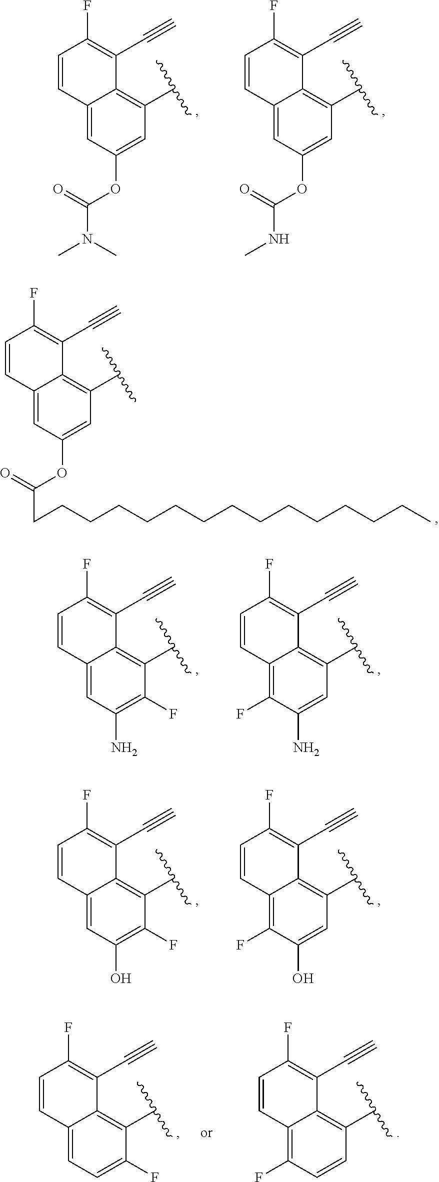

In some embodiments, the compound is an one of the following formulas:

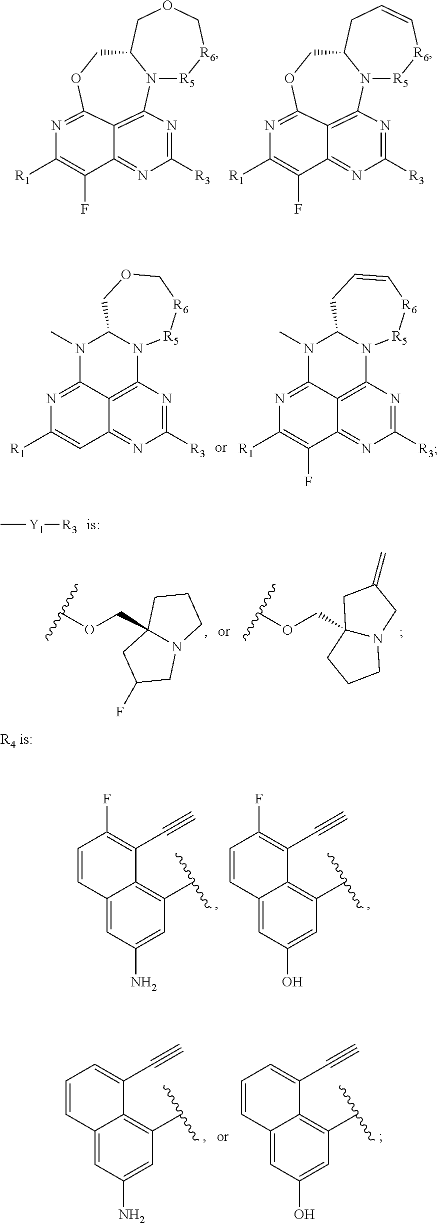

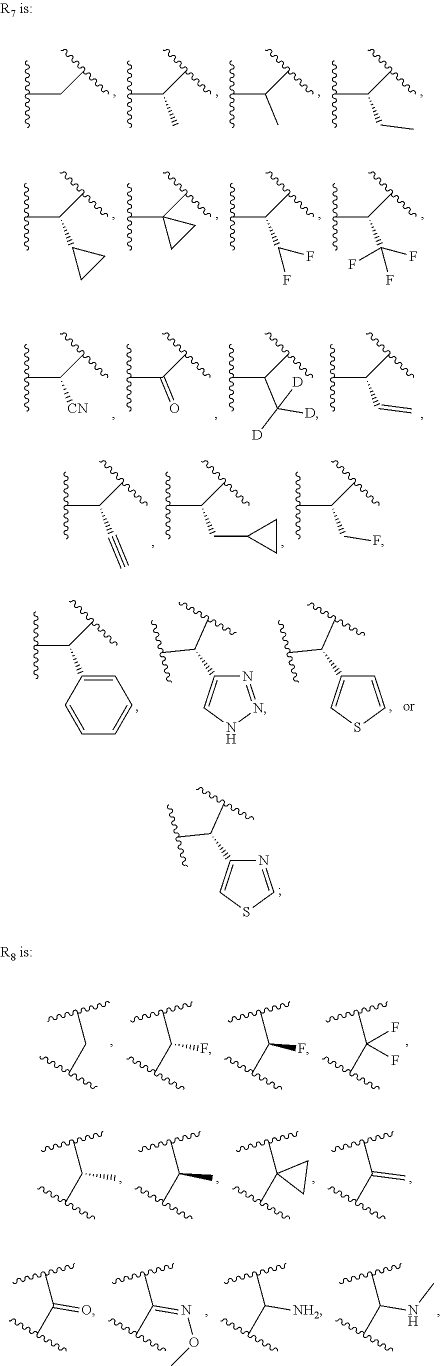

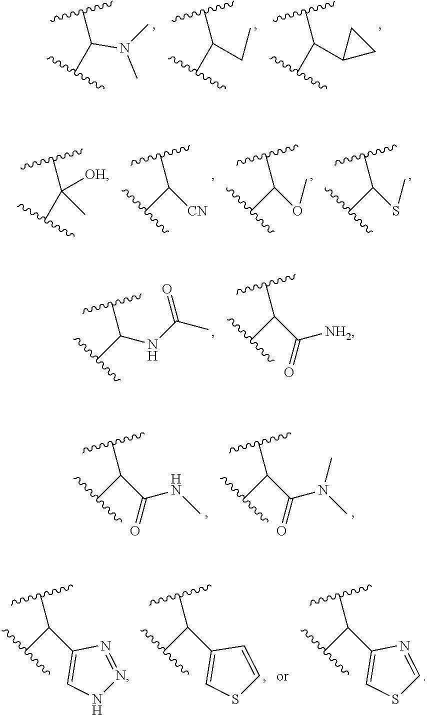

In some embodiments, the compound is any one of the following formulas:

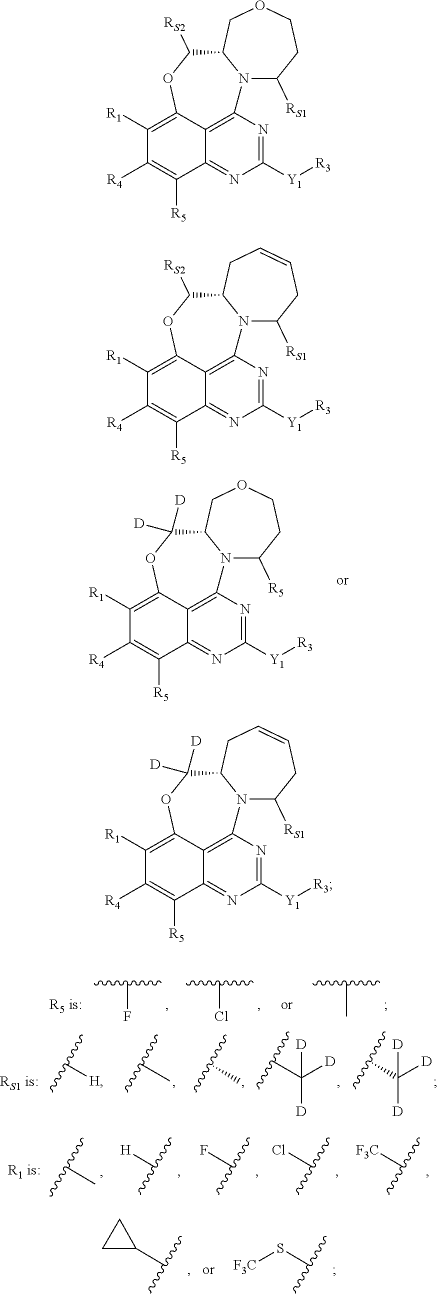

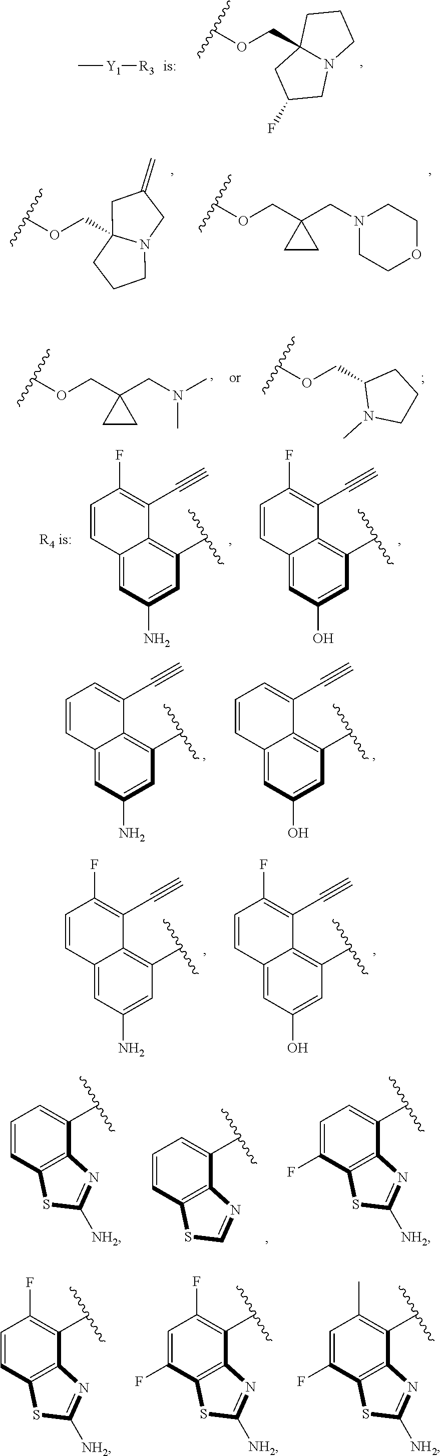

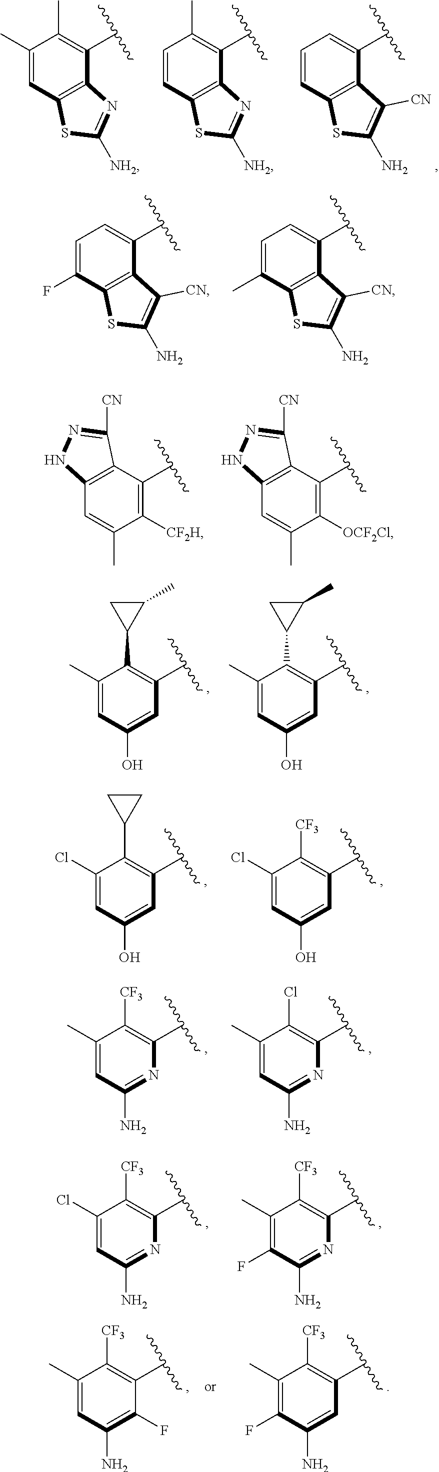

In some embodiments, the compound is any one of the following formula:

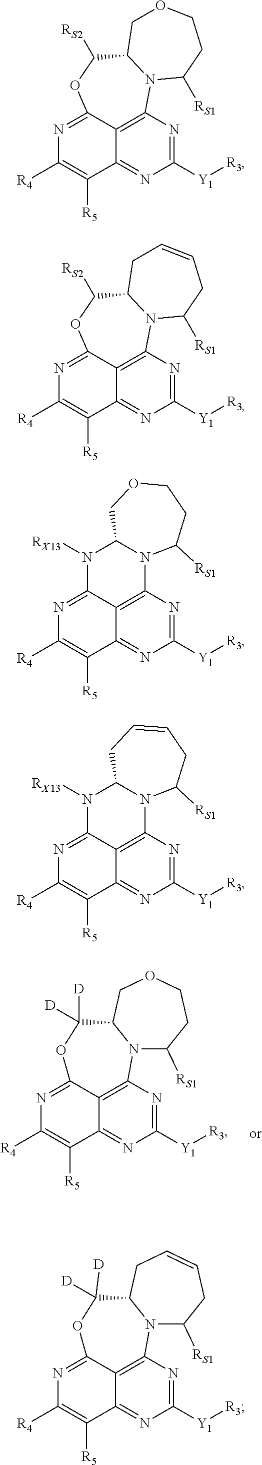

Wherein,
R₅ is —F, —Cl, —CH₃ or —OCH₃;
R_{X13} is —CH₃ or —CD₃;
R_{S2} is —H, —CH₃ or —CD₃;
R_{S1} is —H, —CH₃ or —CD₃;
—Y₁—R₃ is:

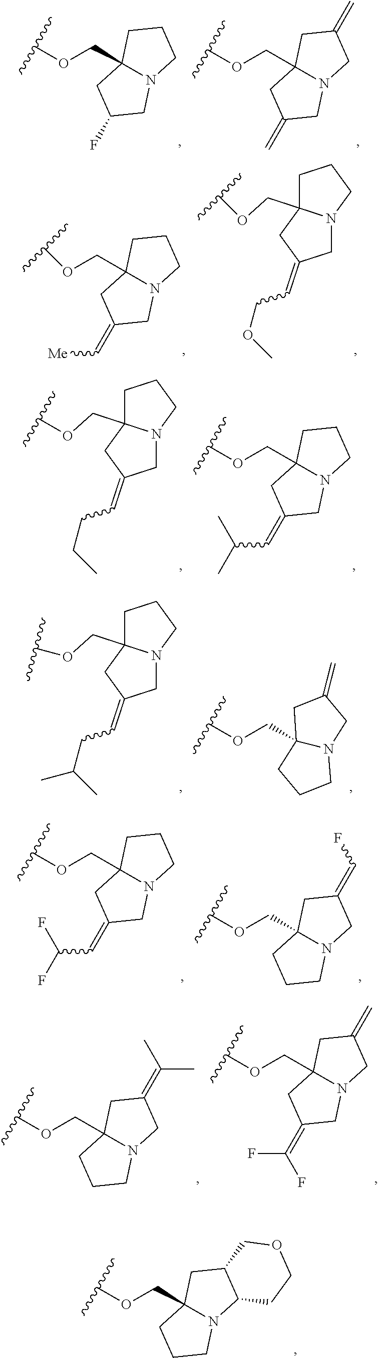

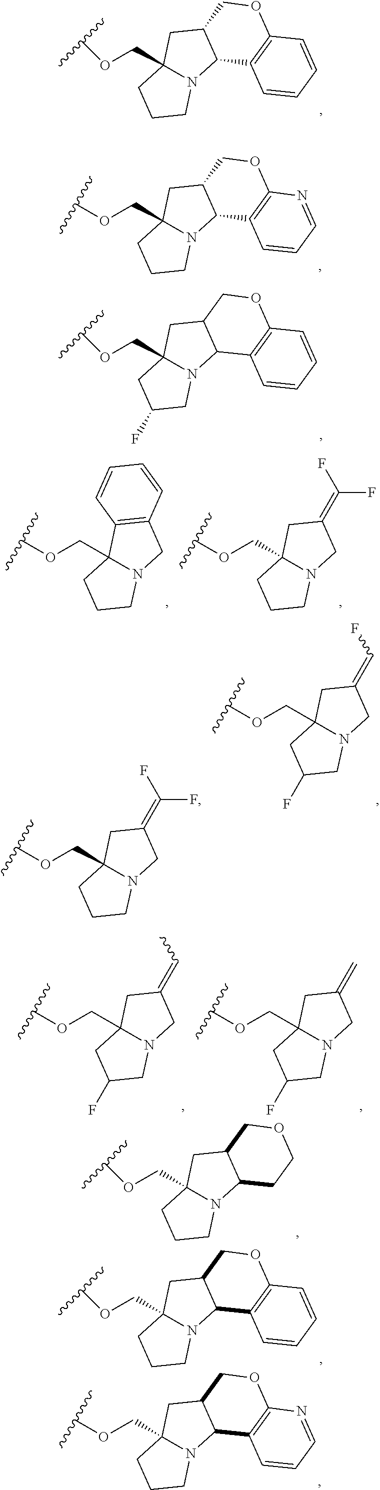

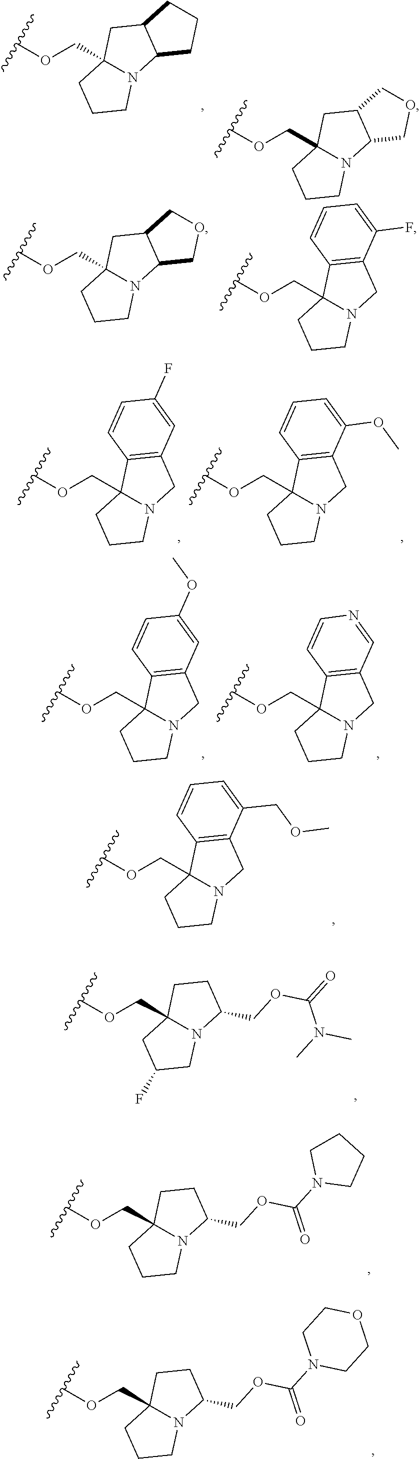

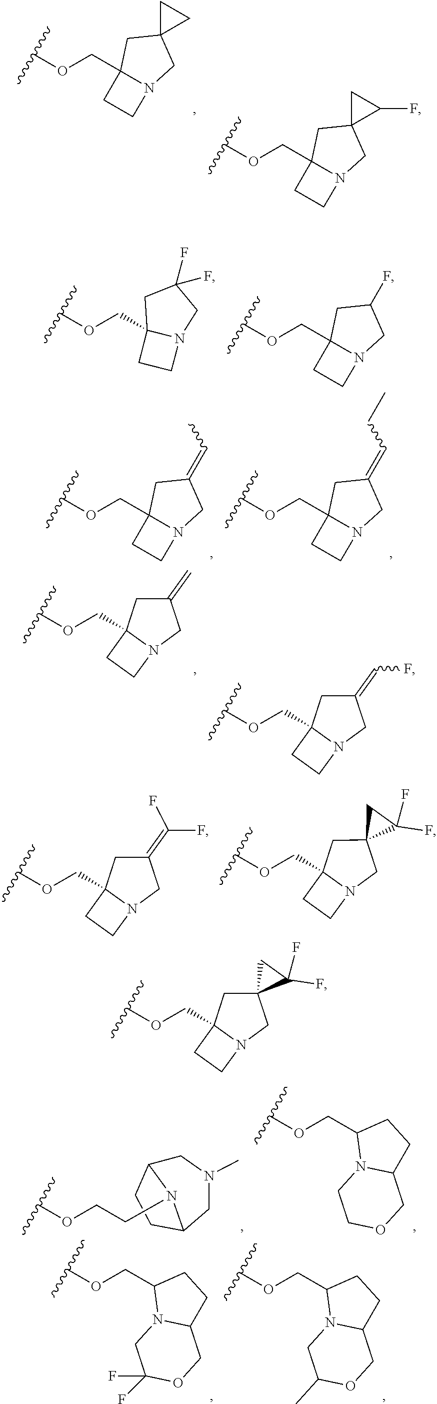

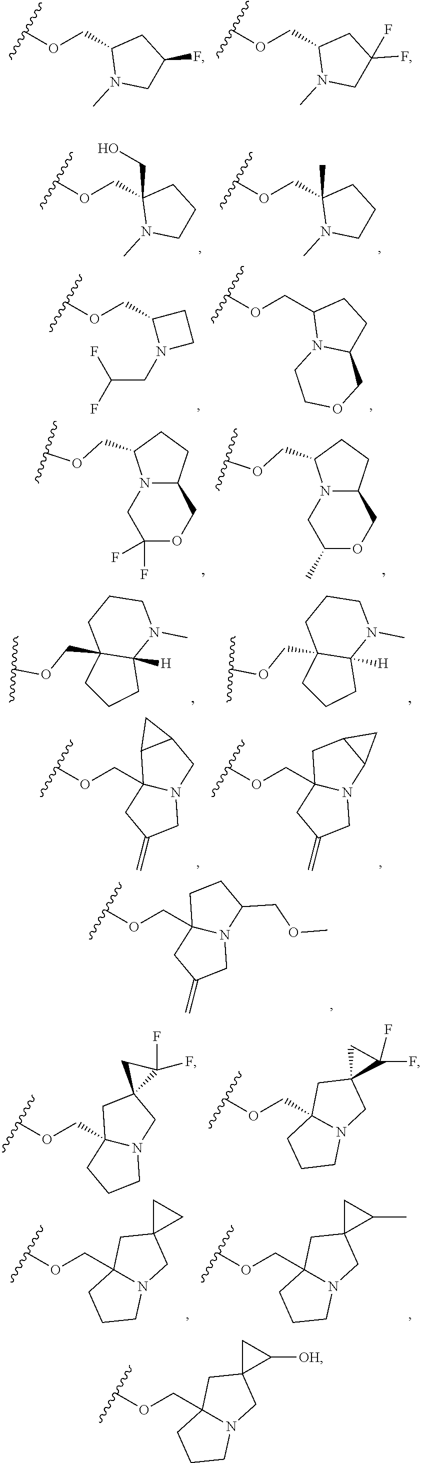

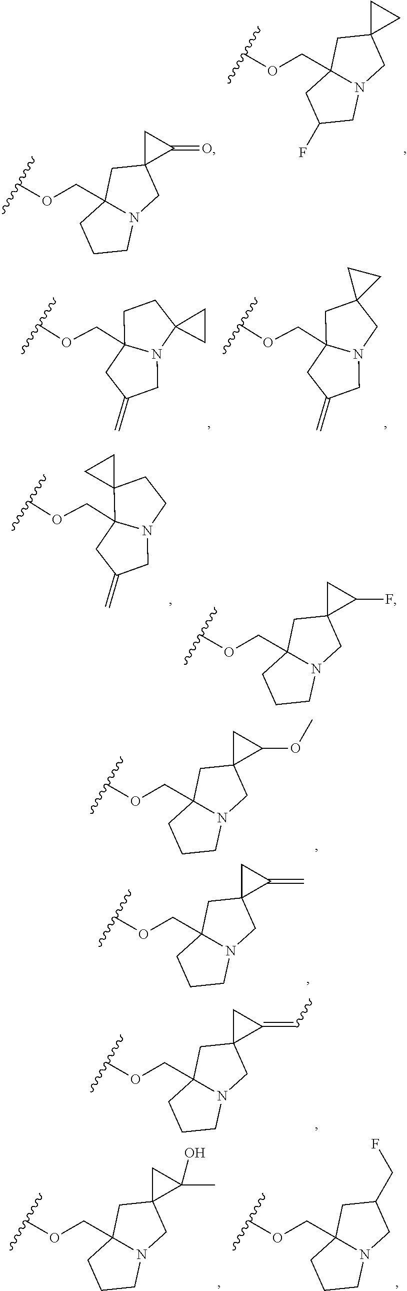

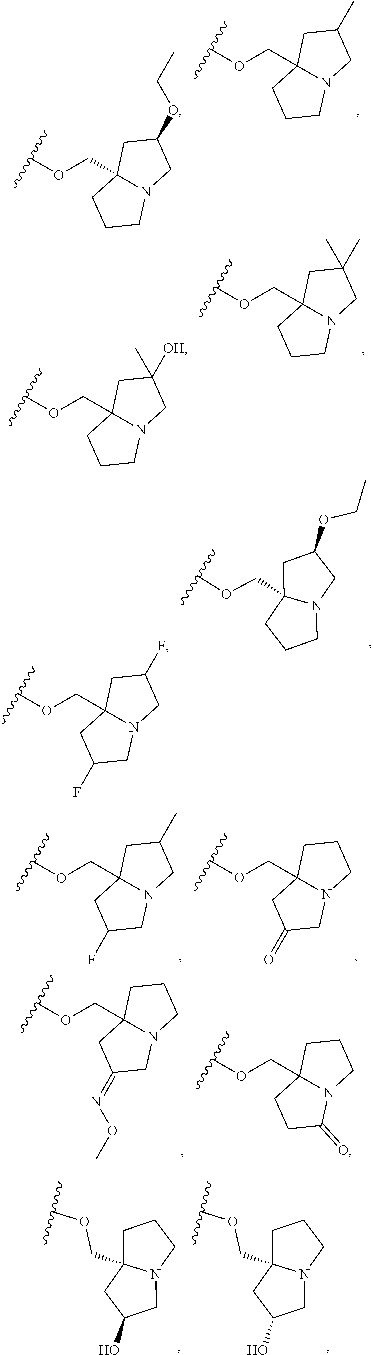

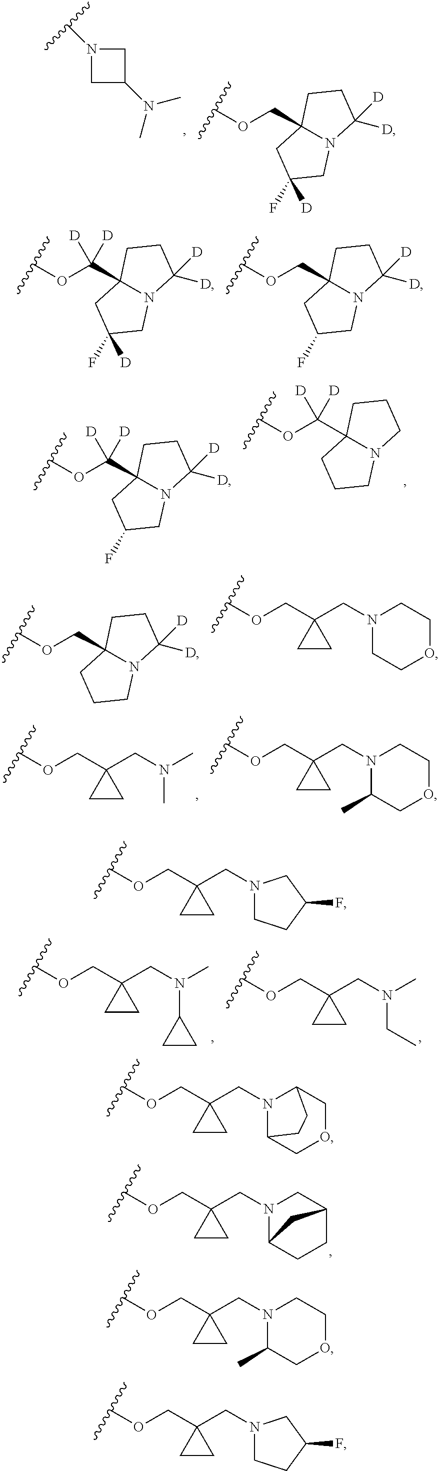

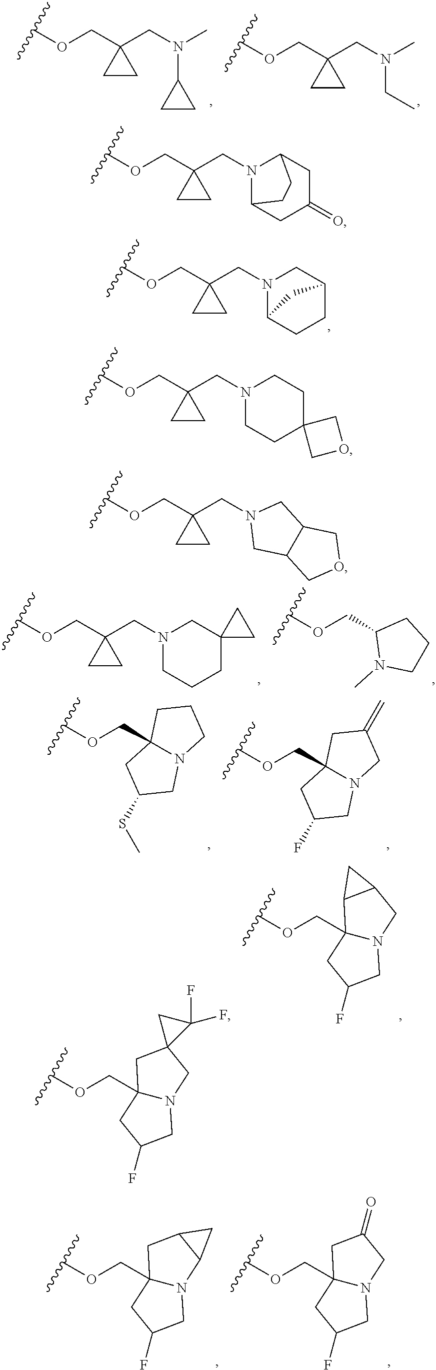

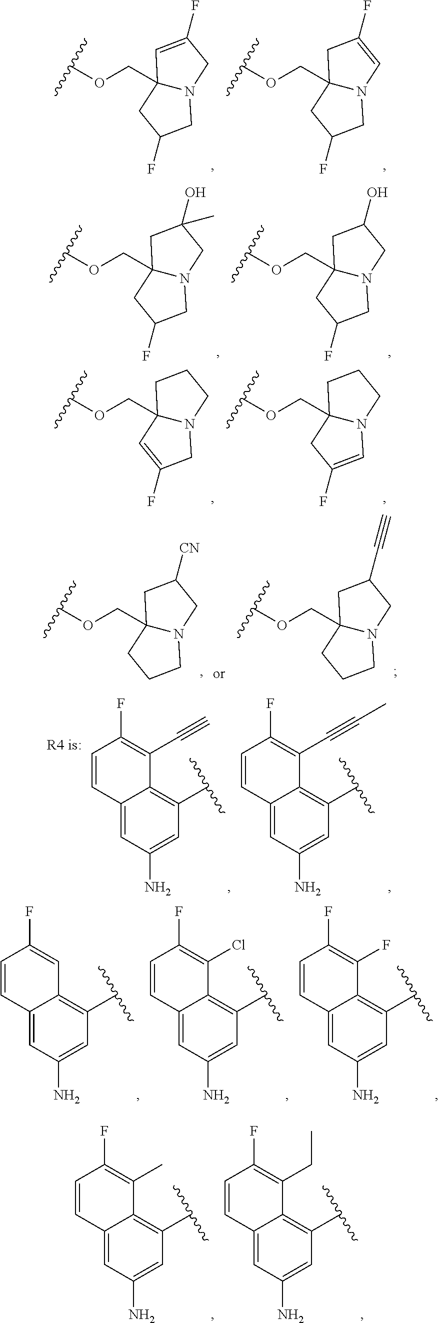

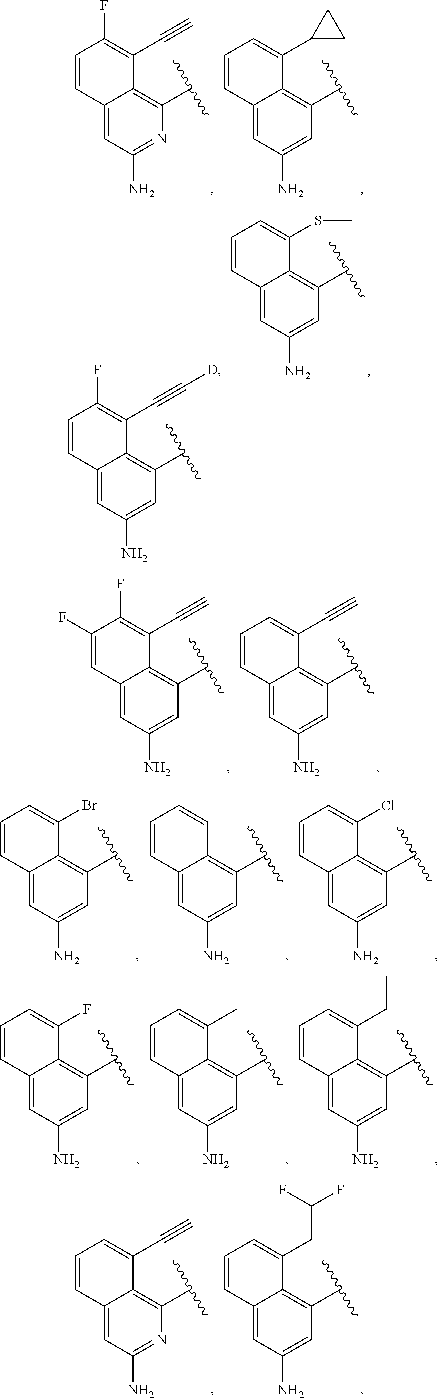

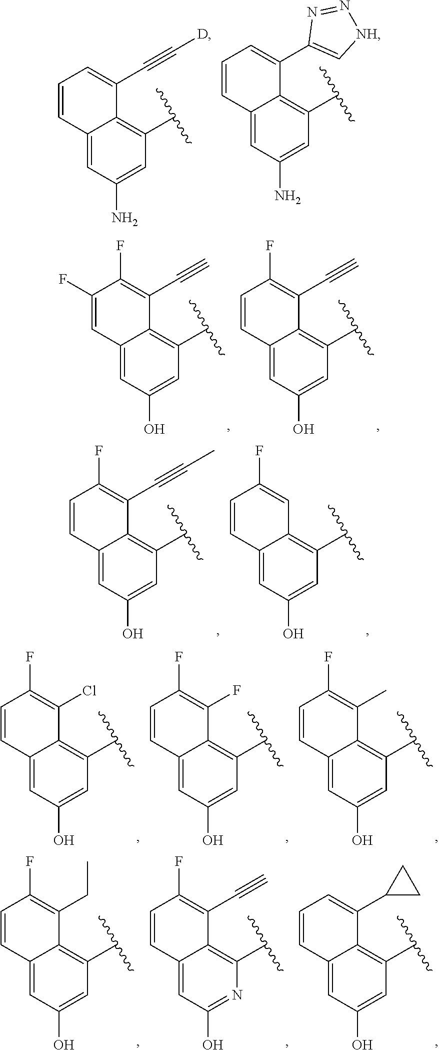

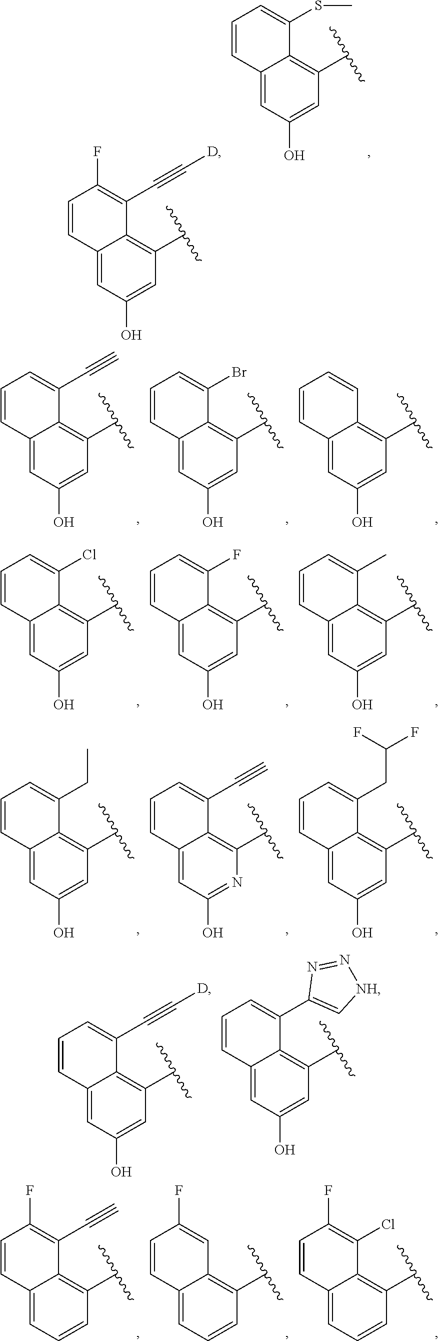

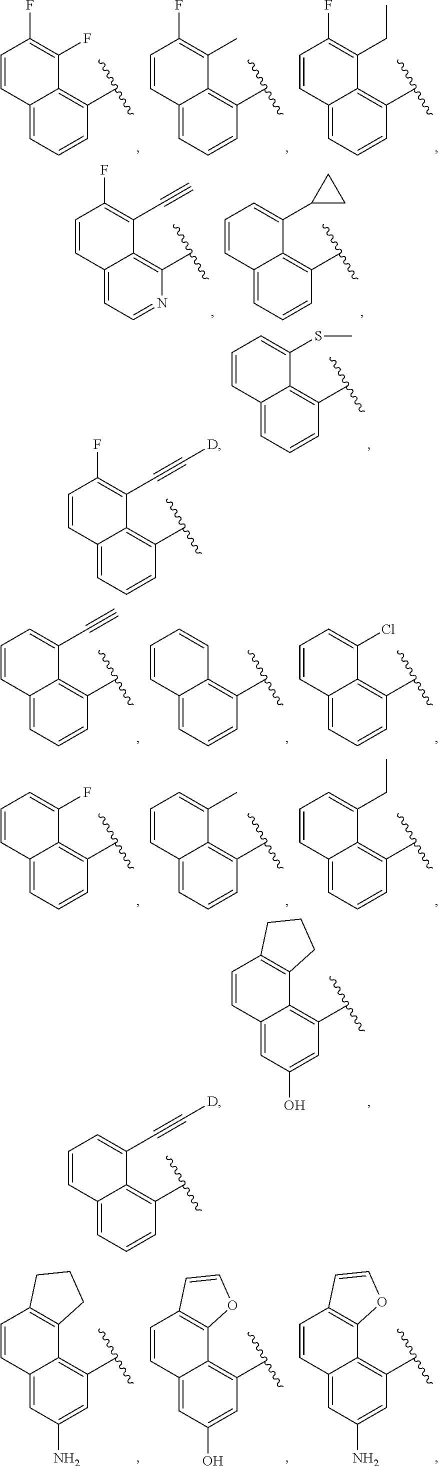

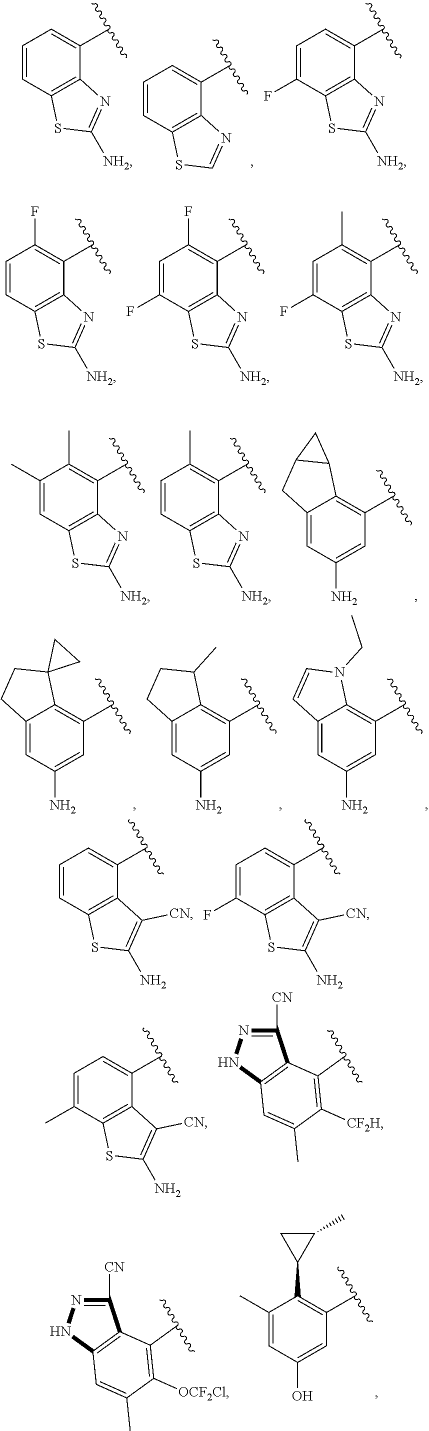

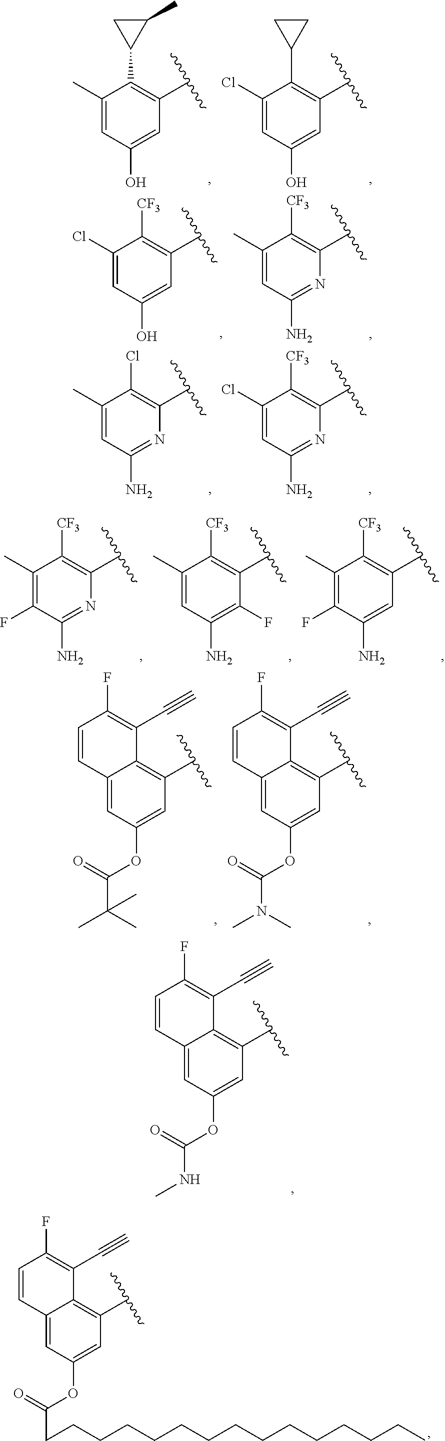

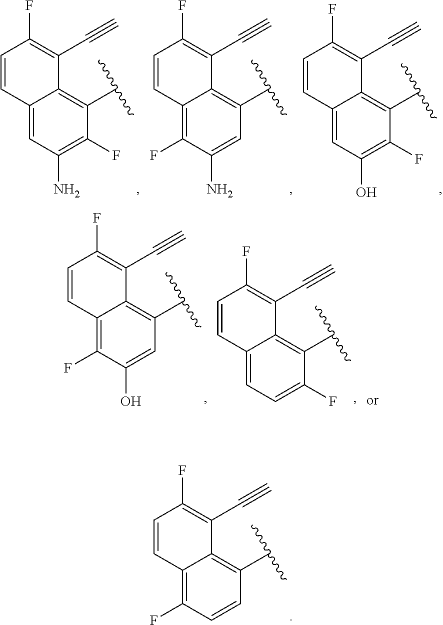

In some embodiments, the compound is:

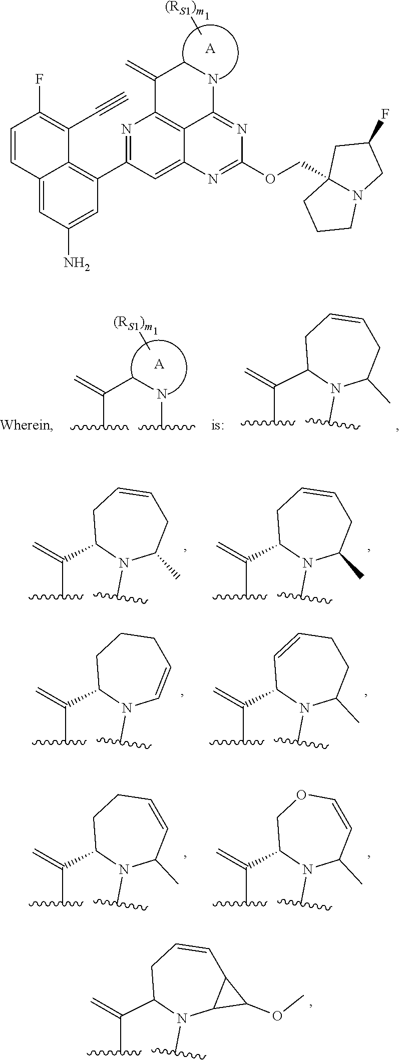

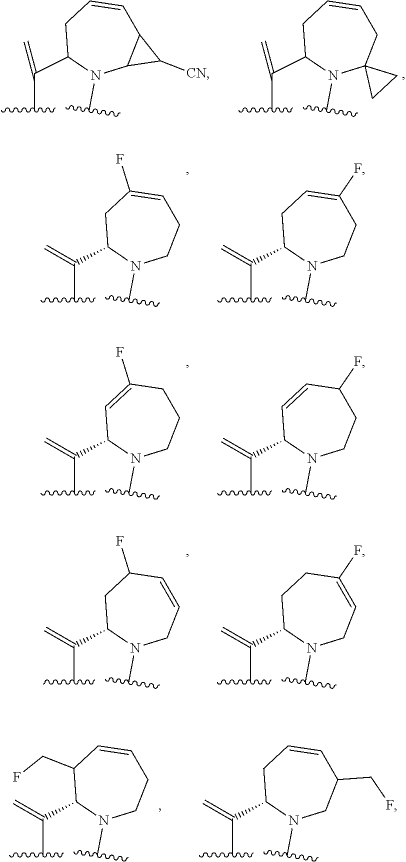

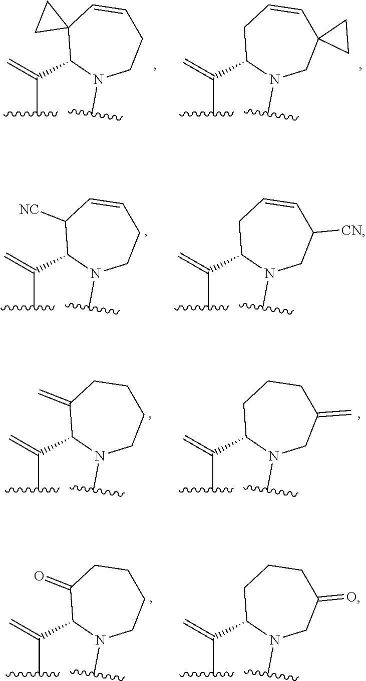

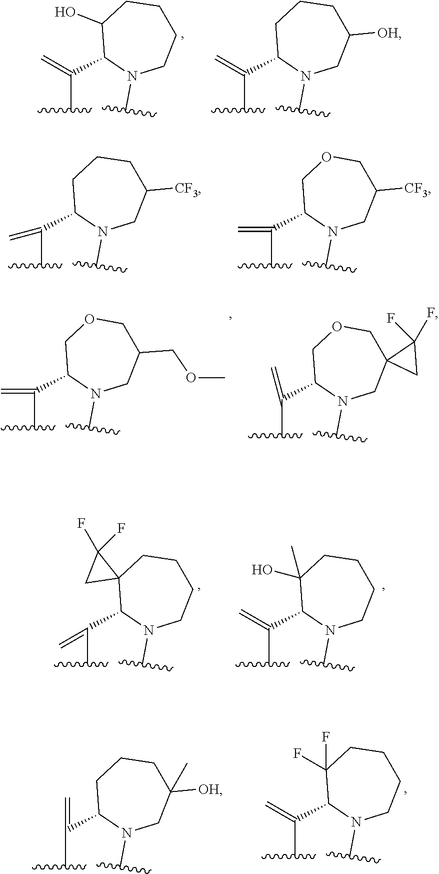

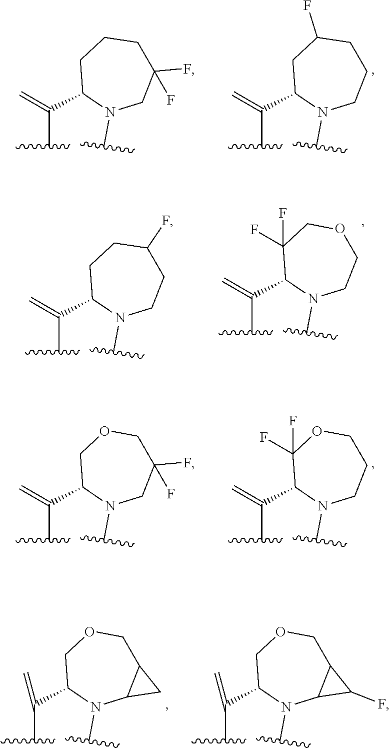

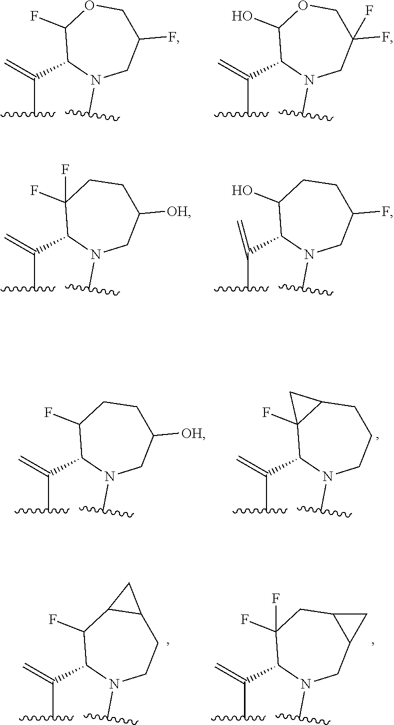

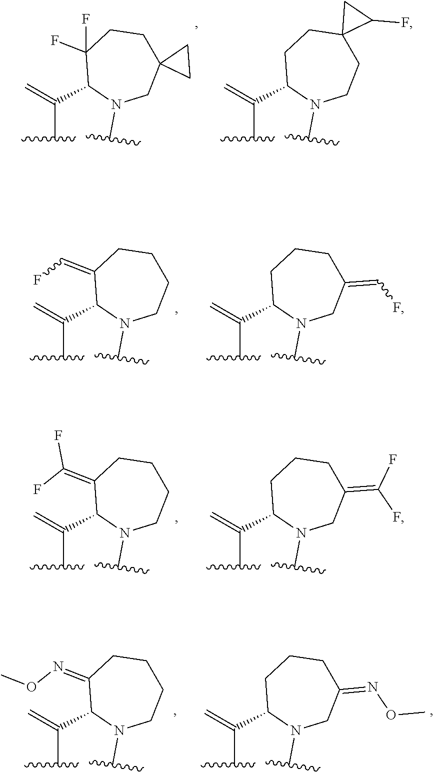

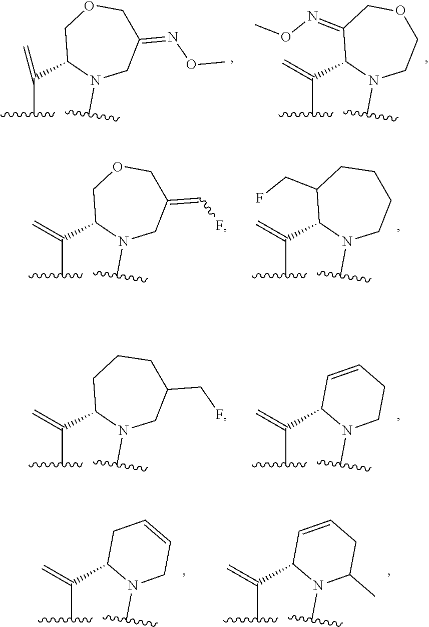

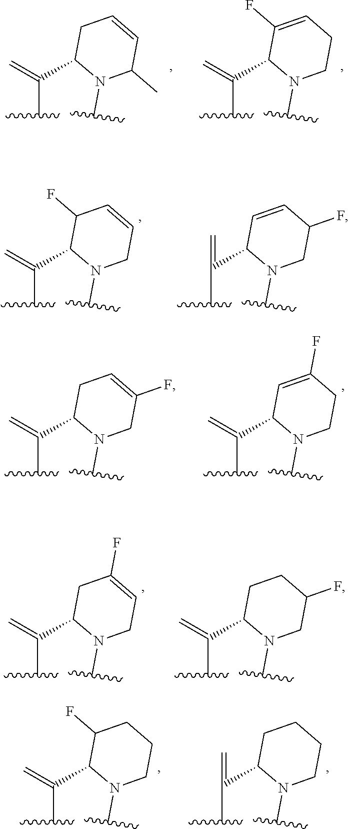

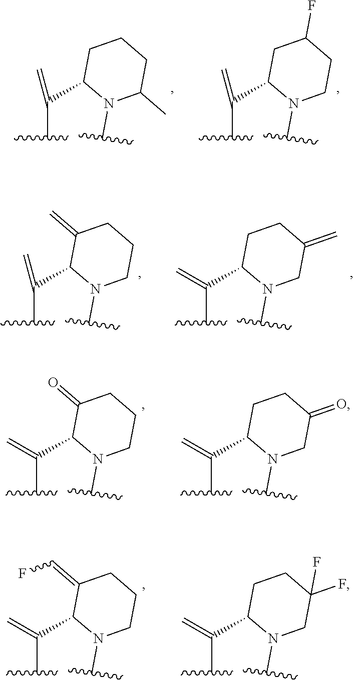

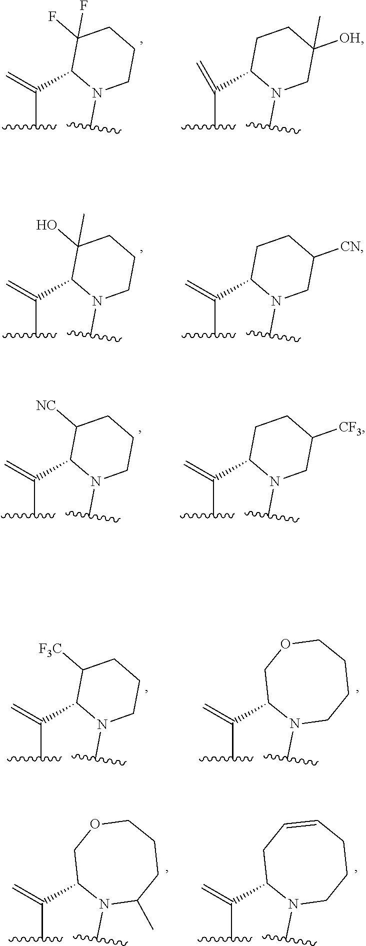

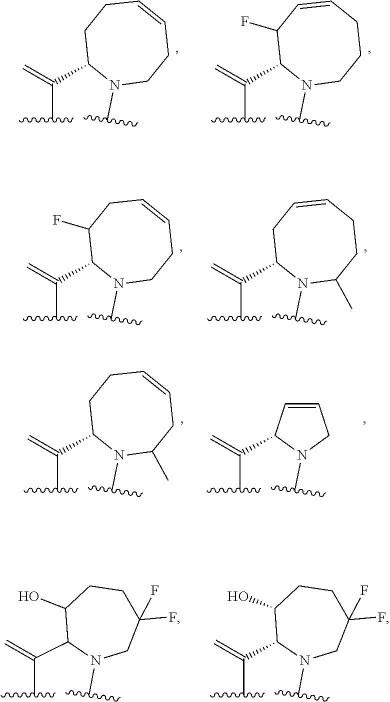

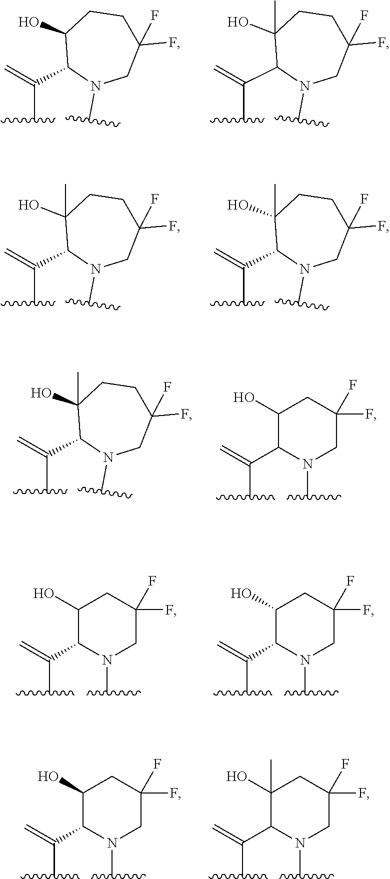

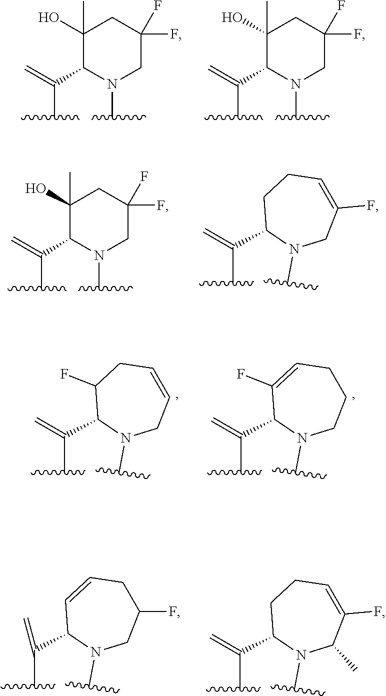

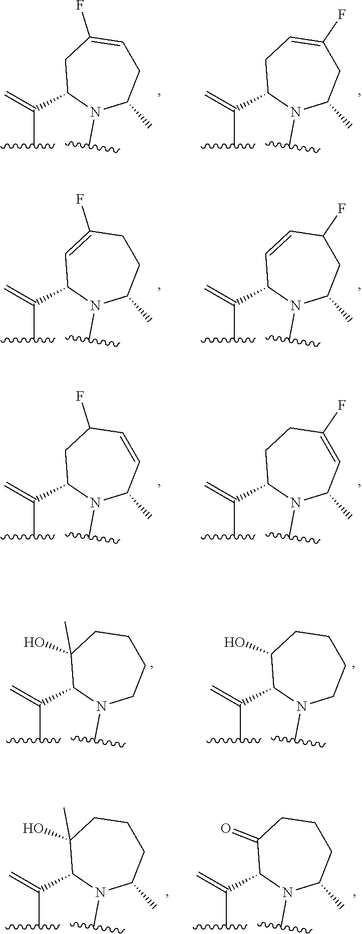

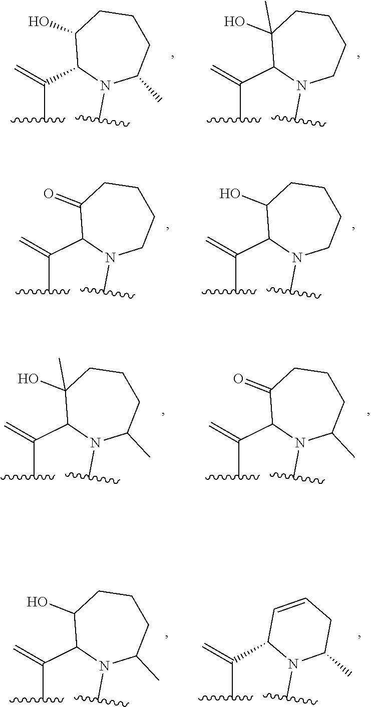

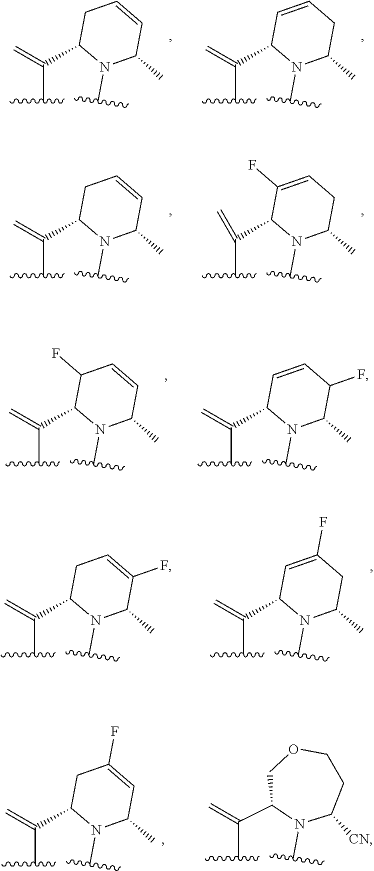

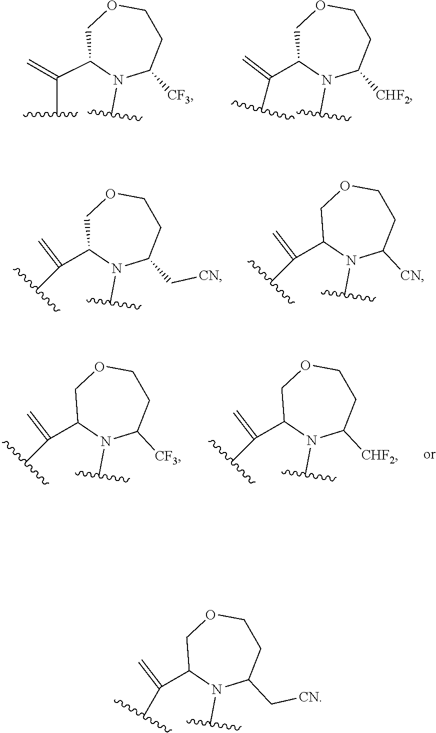

In some embodiments, the compound is:

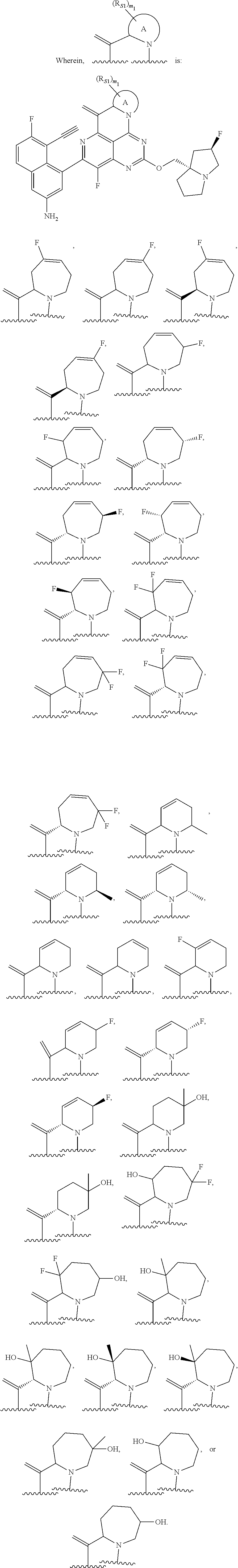

In some embodiments, the compound is:

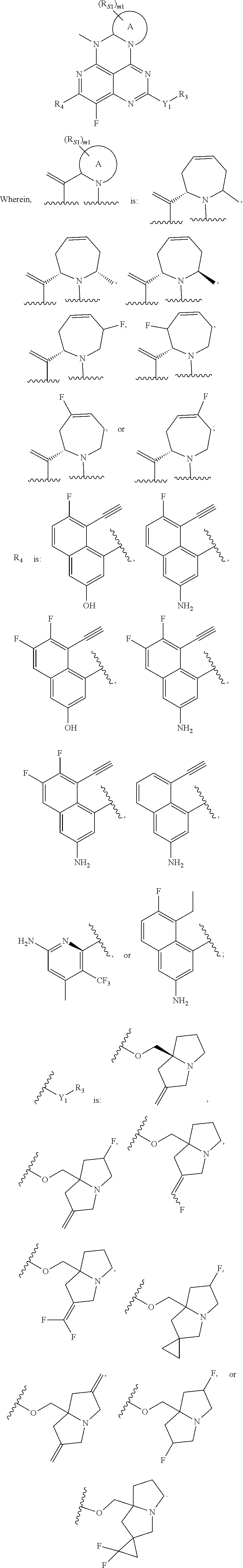

In some embodiments, the compound is:

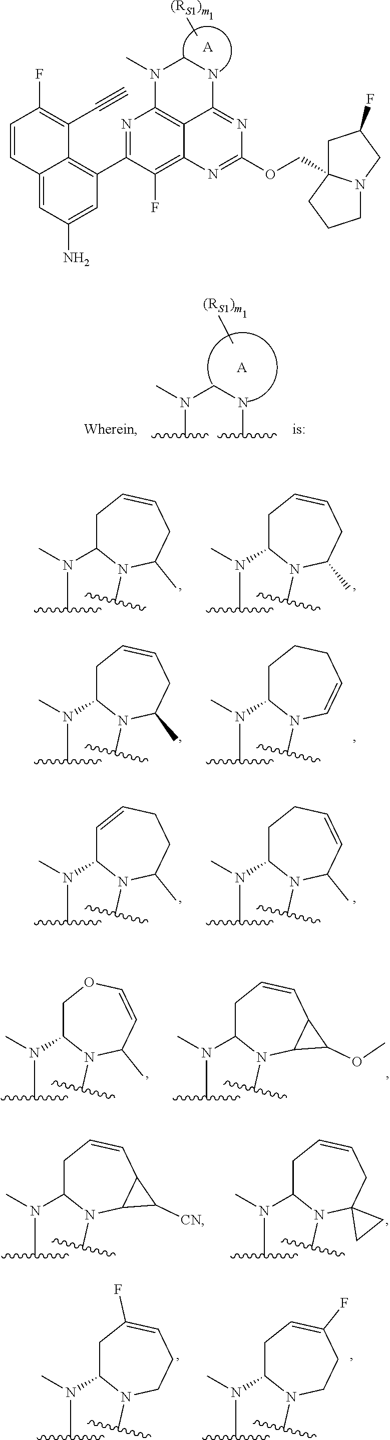

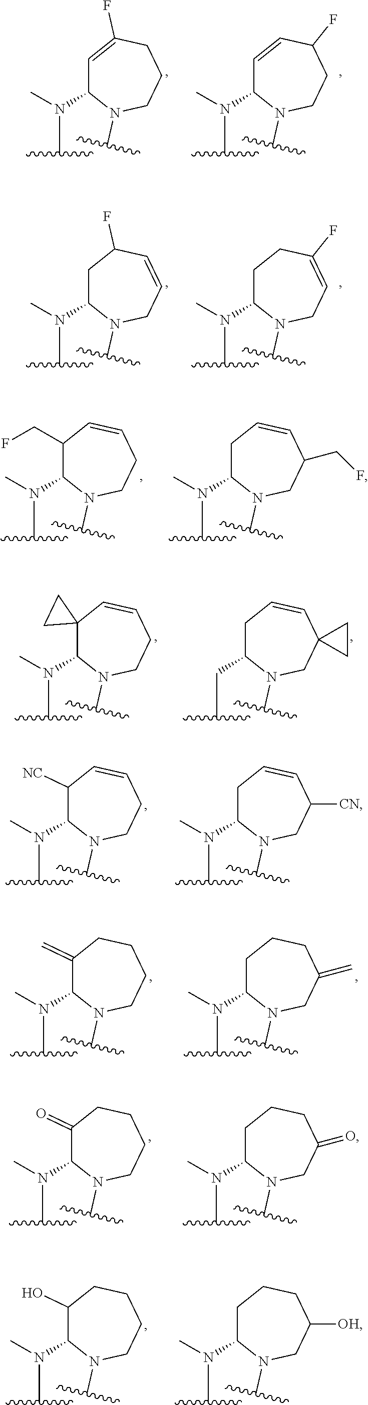

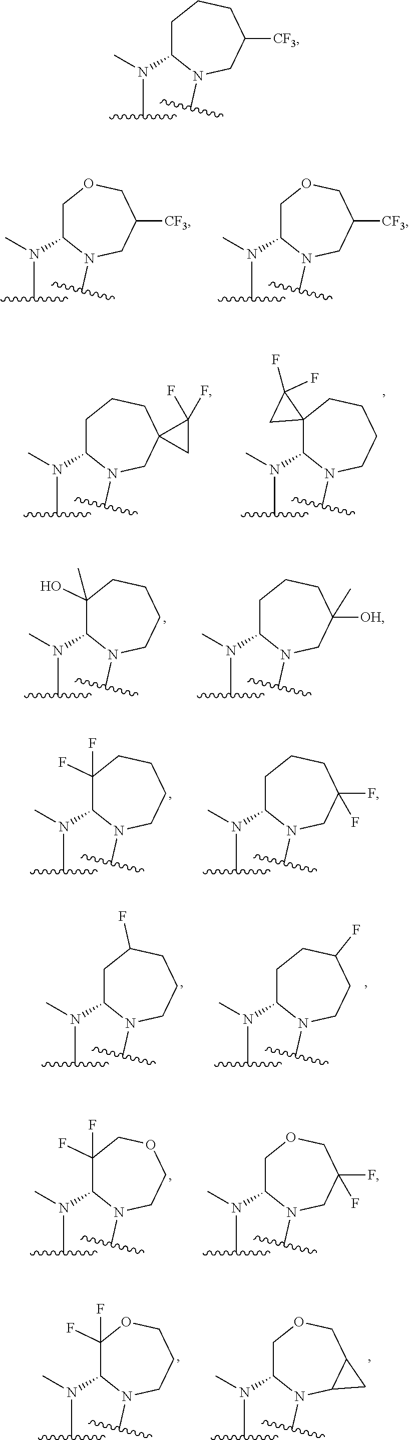

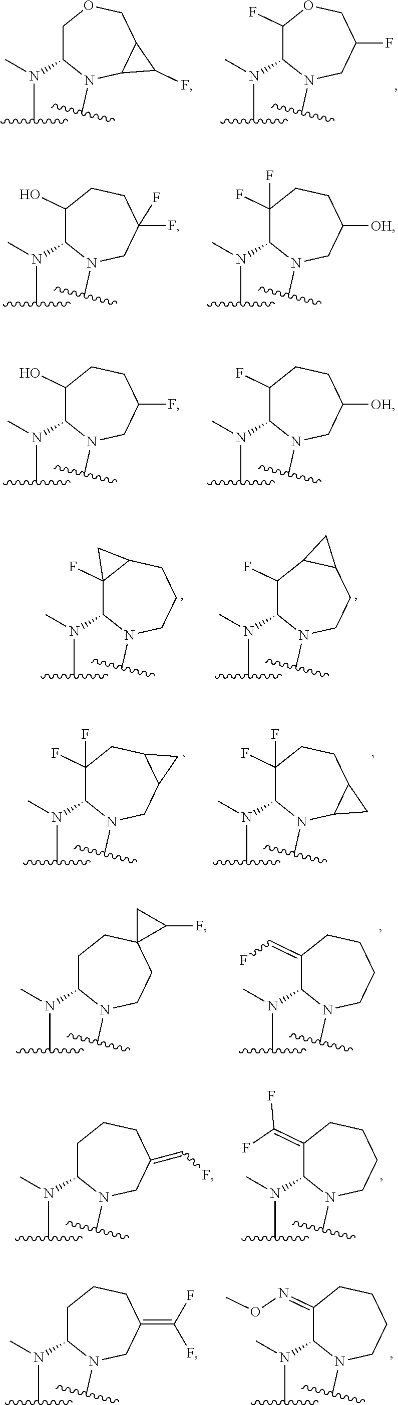

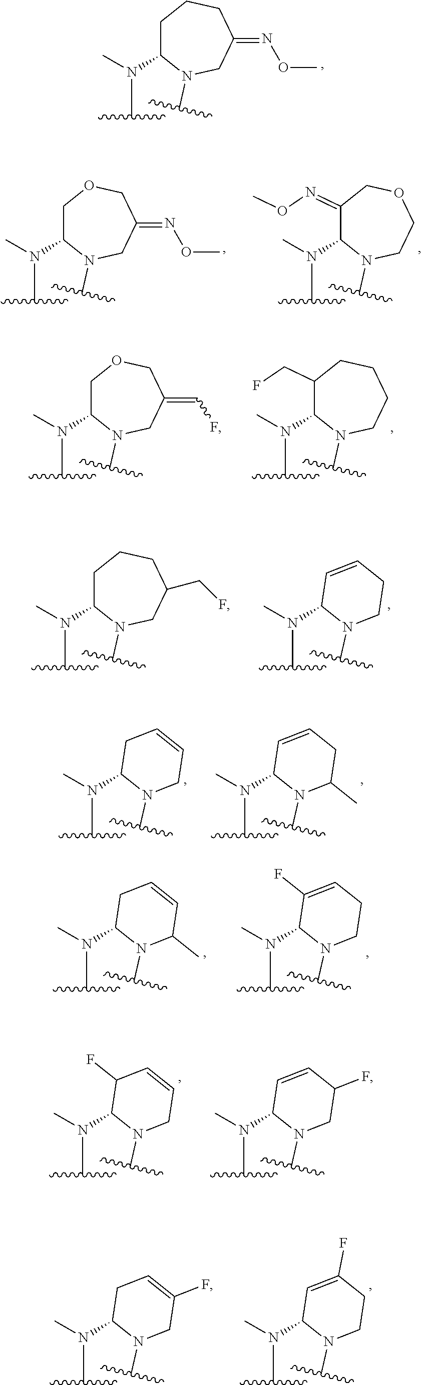

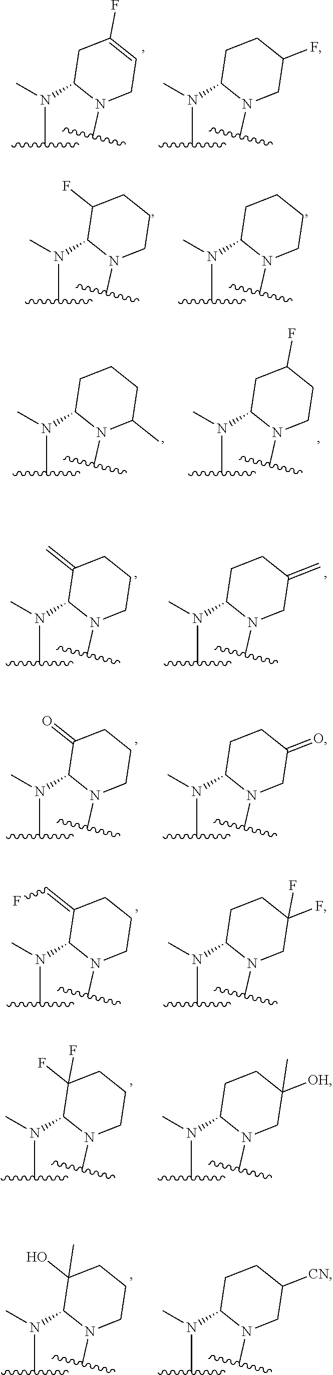

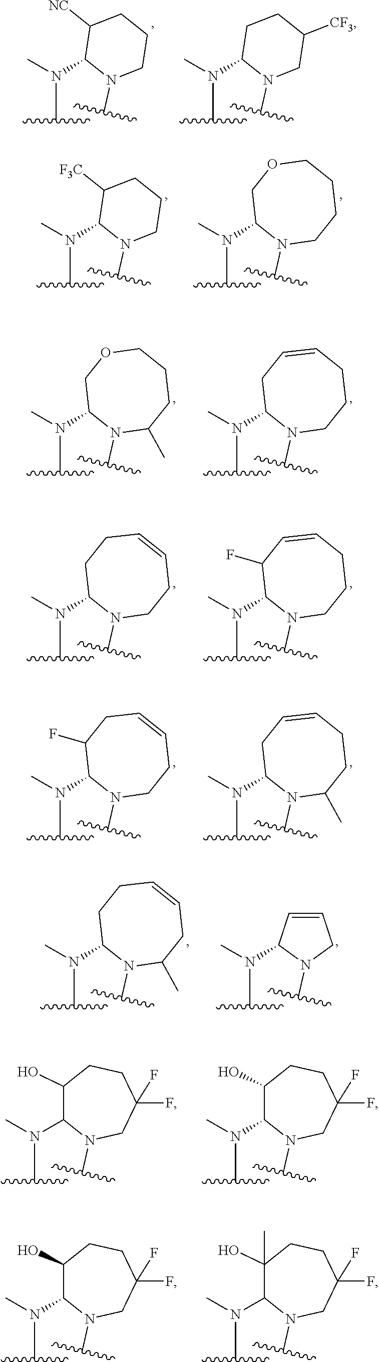

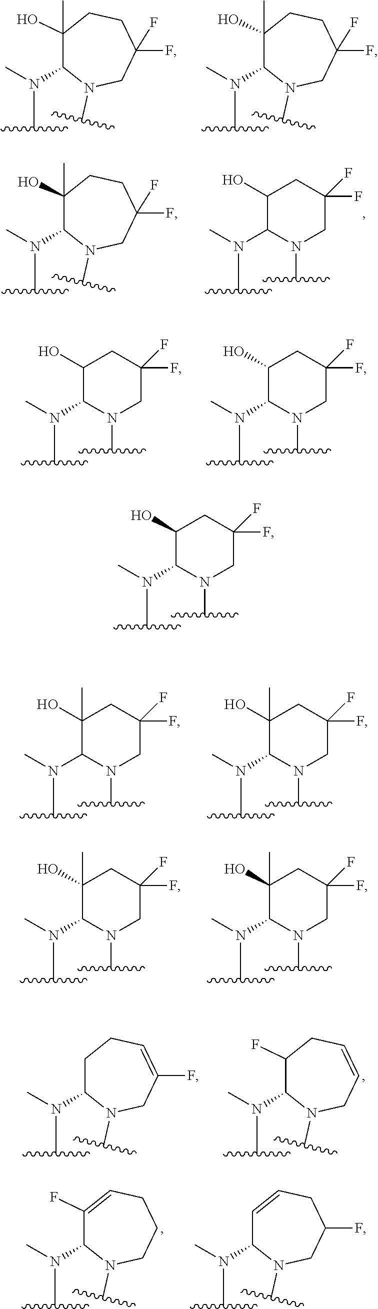

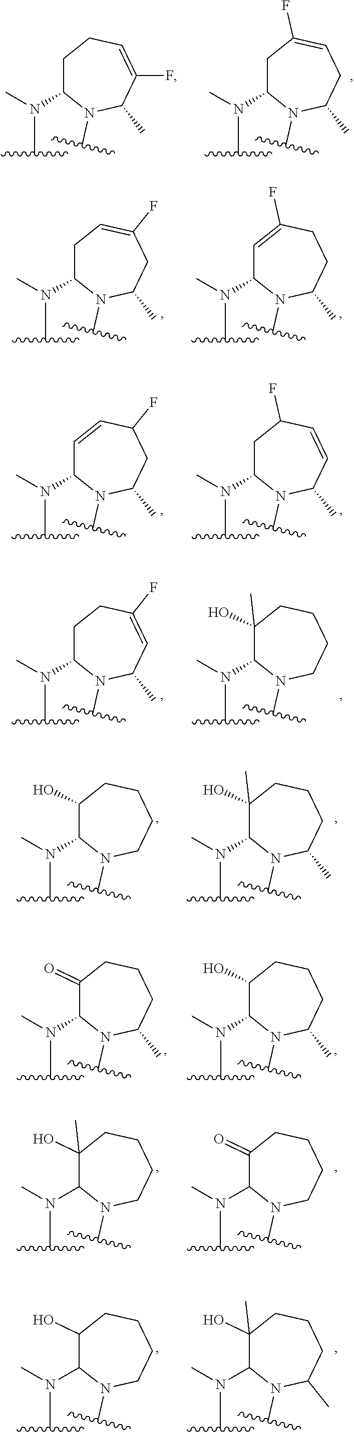

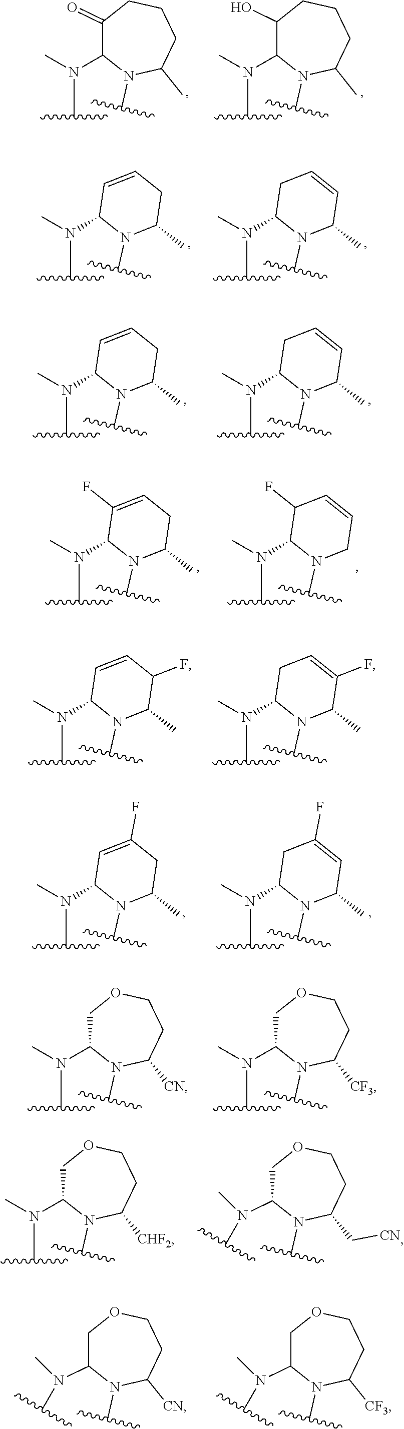

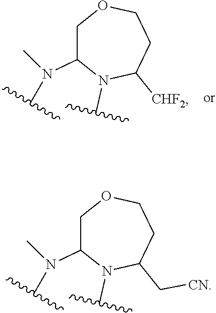 [SMILES: *N(C)C1COCCC(C(F)F)N1* ; "or" ; *N(C)C1COCCC(CC#N)N1*]

In some embodiments, the compound is:

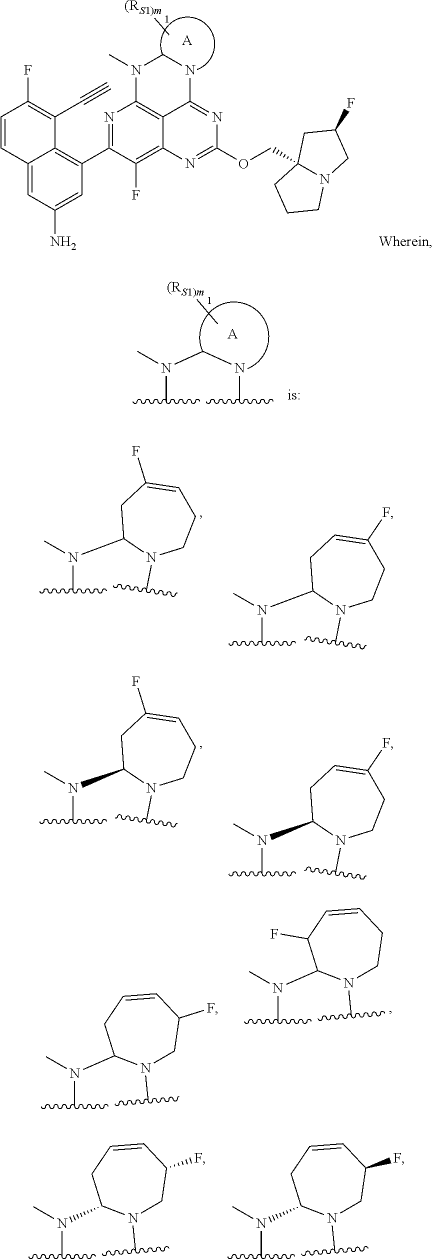

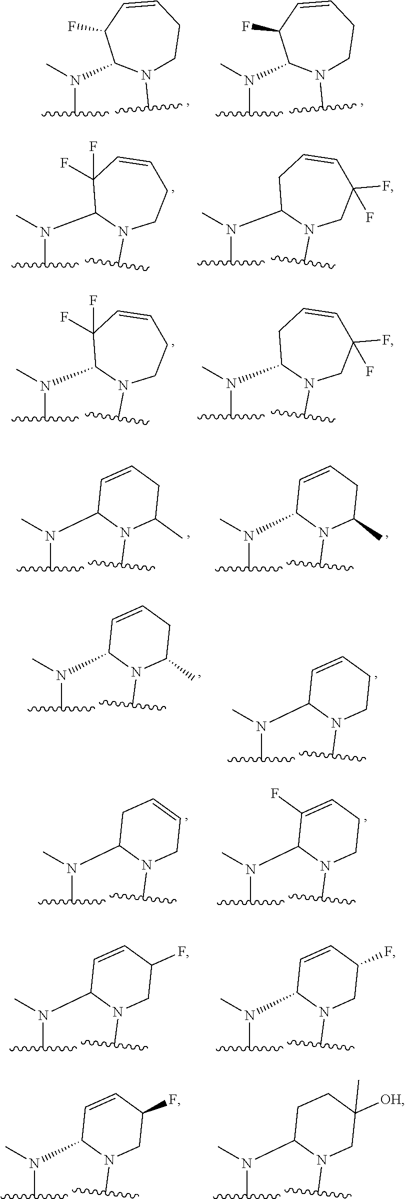

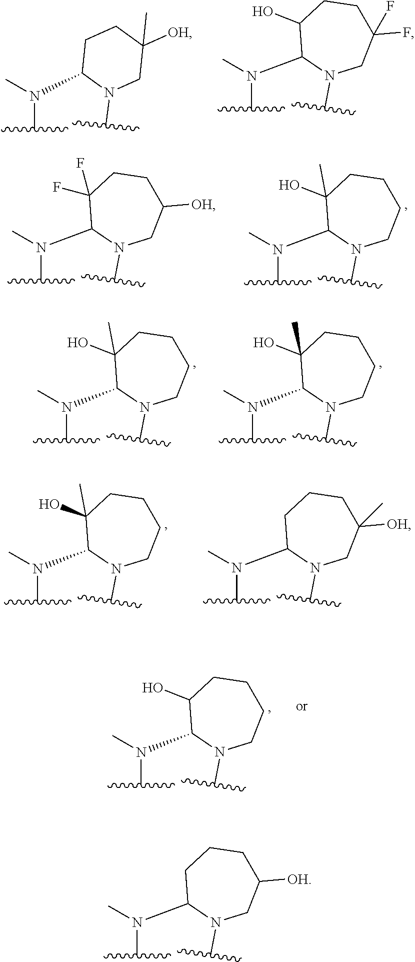

In some embodiments, the compound is:

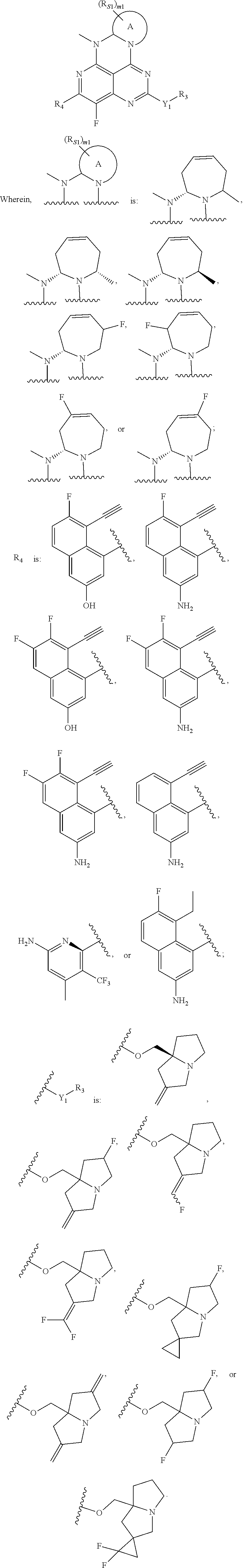

In some embodiments, the compound is:

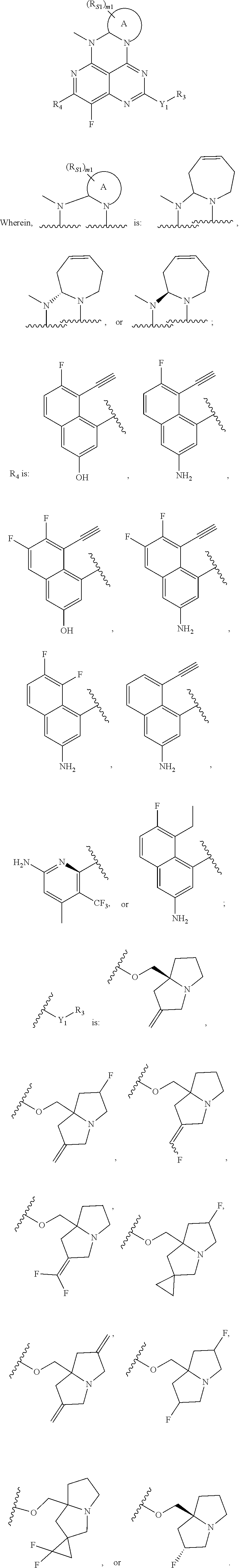

In some embodiments, the compound is:

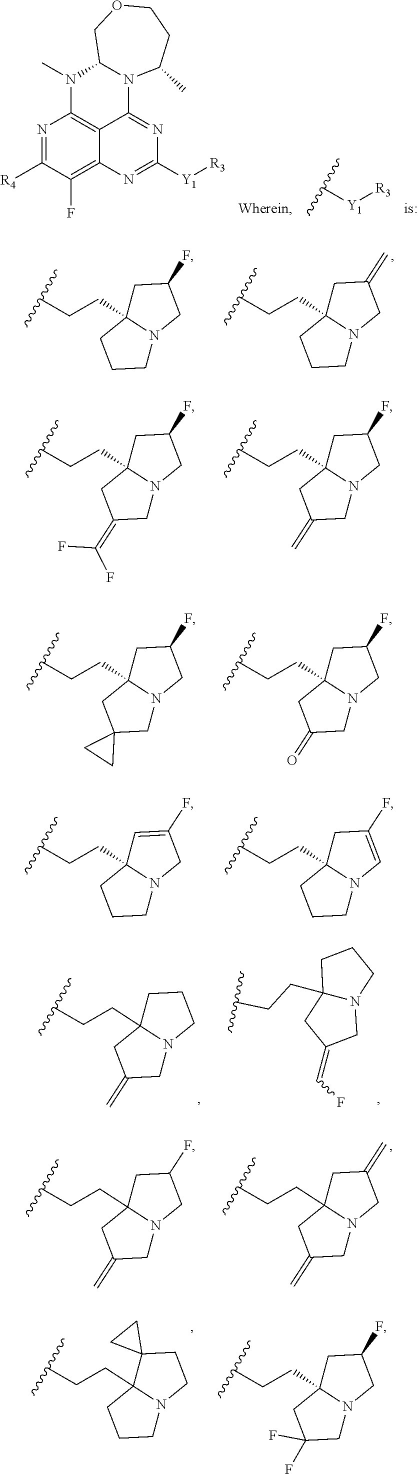

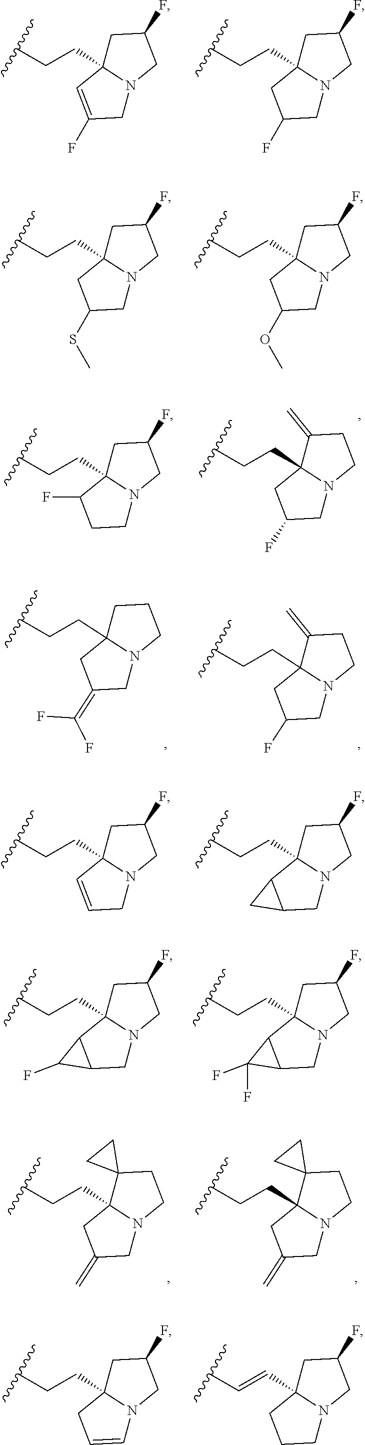

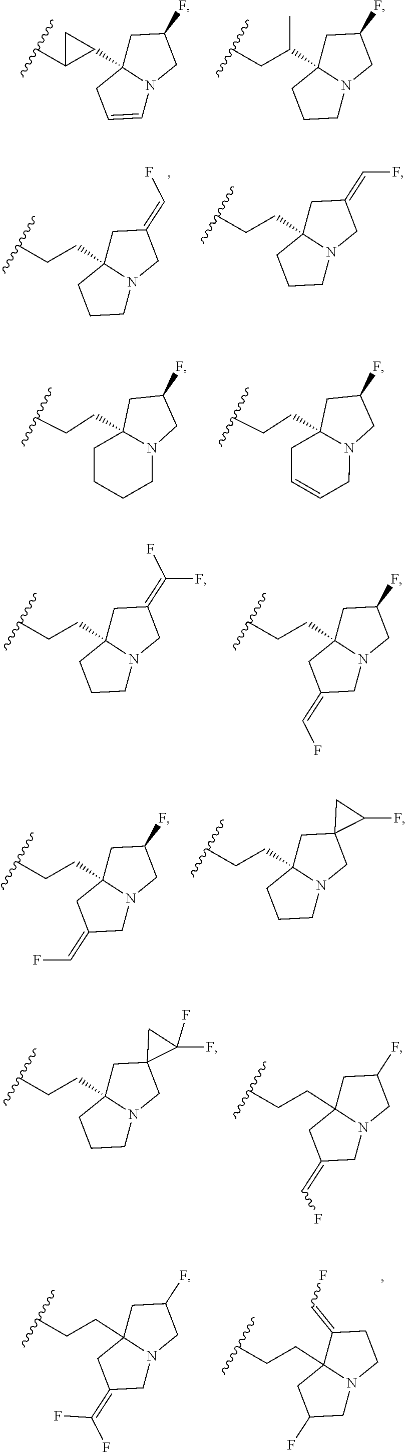

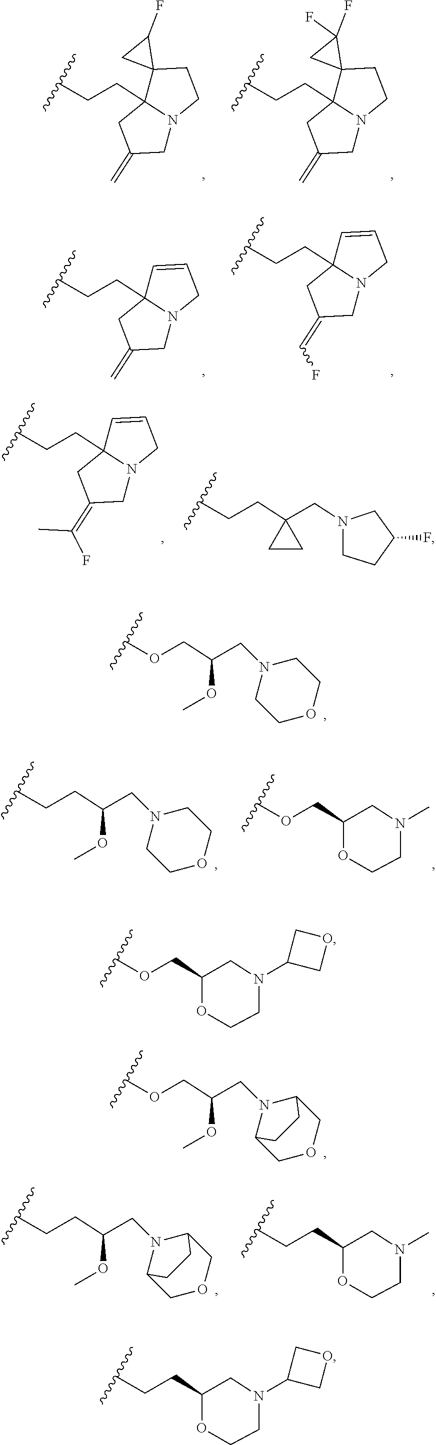

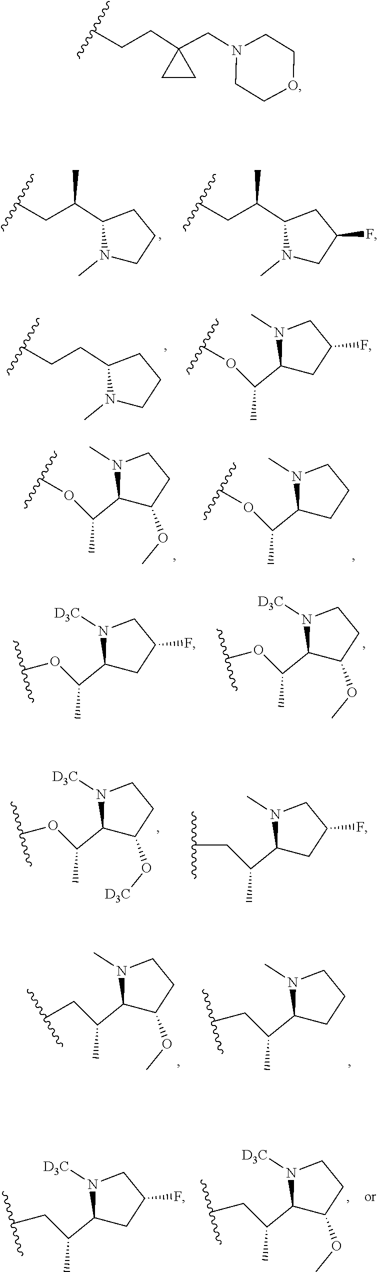

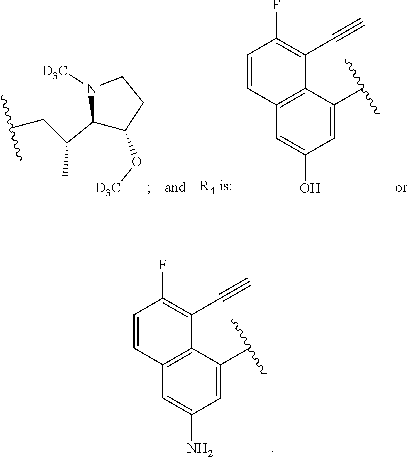 [SMILES: *C[C@@H](C)[C@@H]1[C@@H](OC([2H])([2H])[2H])CCN1C([2H])([2H])[2H] ; *c1cc(O)cc2ccc(F)c(C#C)c12 ; *c1cc(N)cc2ccc(F)c(C#C)c12]

In some embodiments, the compound is:

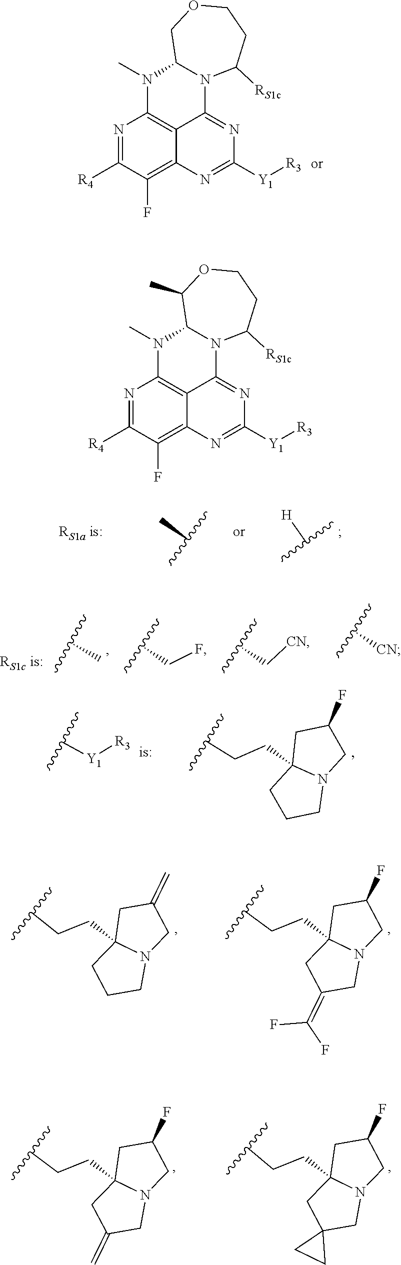

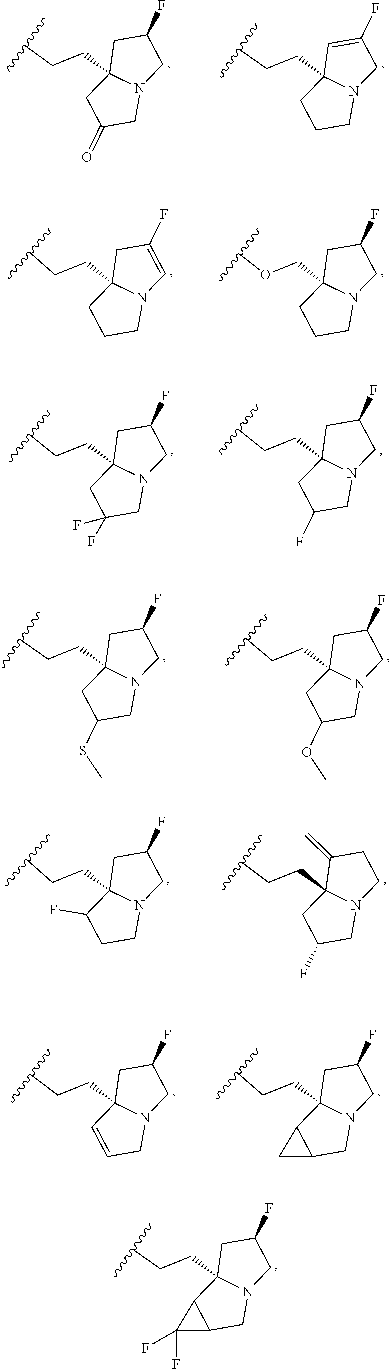

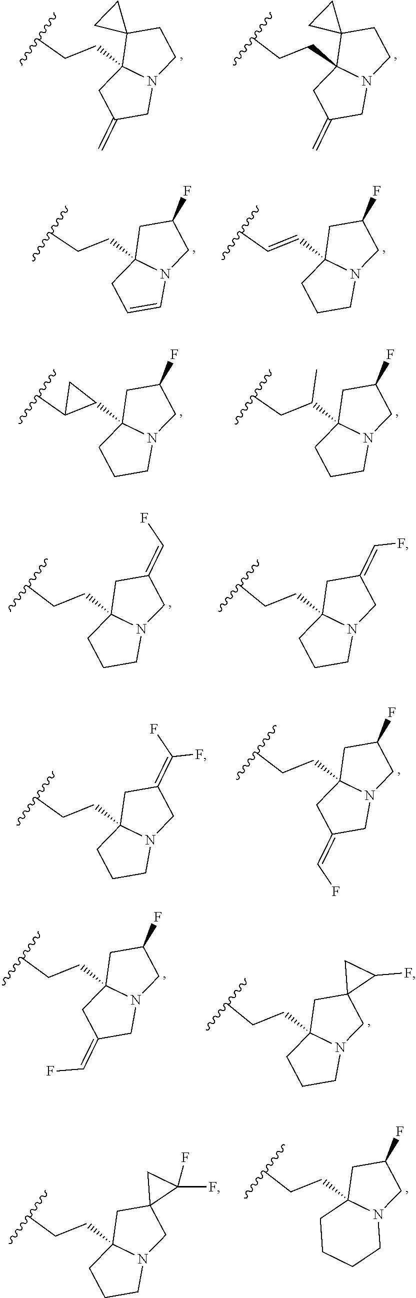

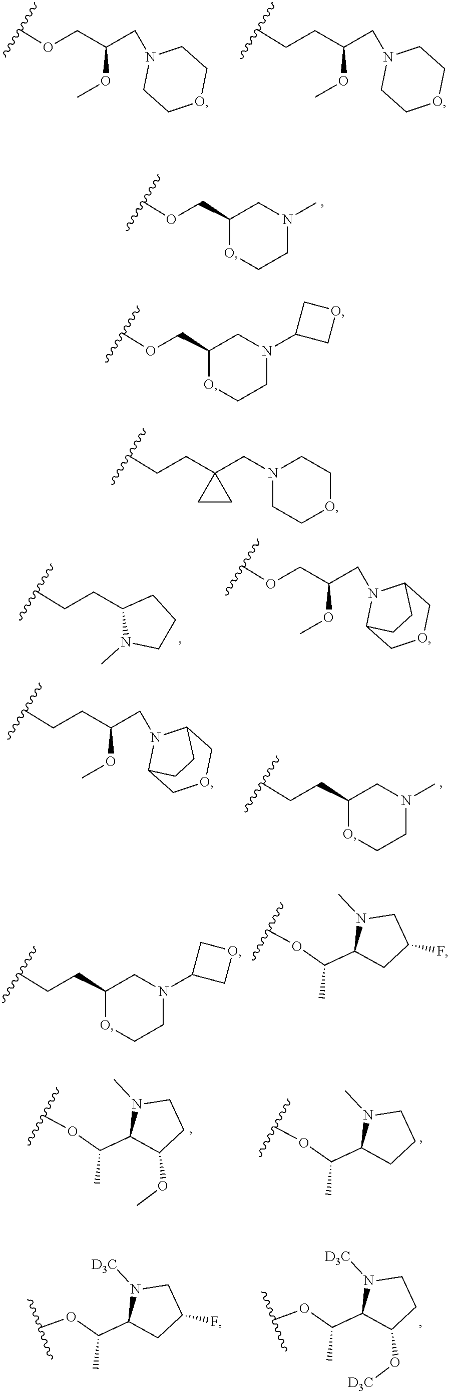

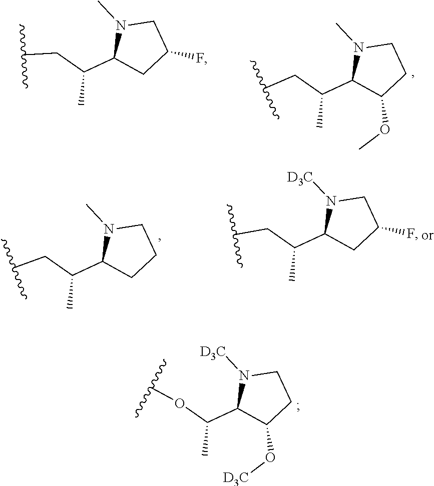

Wherein, R_{S1a} is:
and
R₄ is:

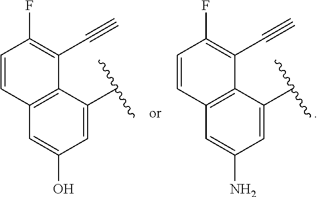 [SMILES: *c1cc(O)cc2ccc(F)c(C#C)c12 ; *c1cc(N)cc2ccc(F)c(C#C)c12]

In some embodiments, the compound is:

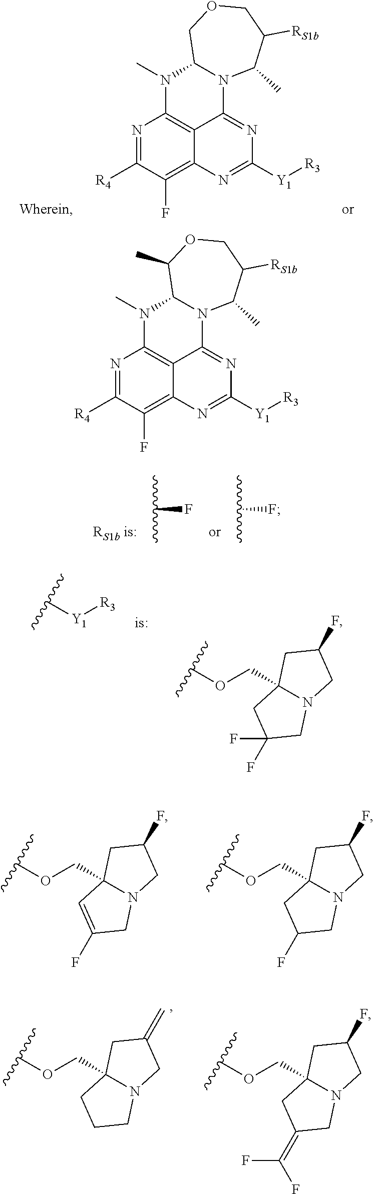

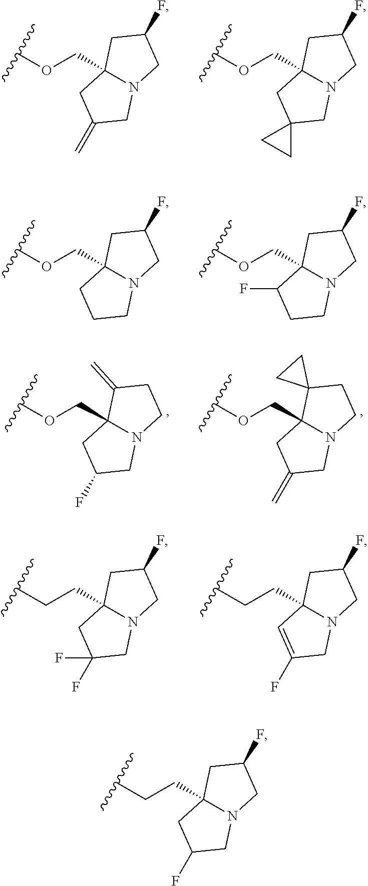

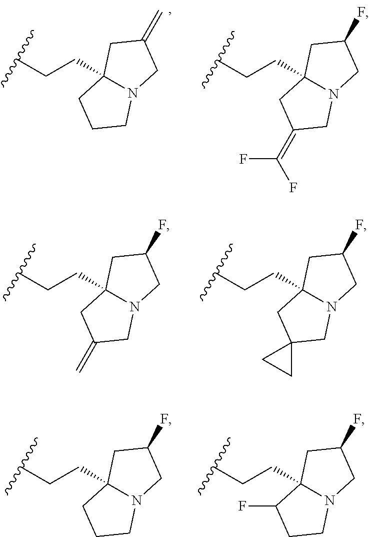 [SMILES: *CC[C@@]12CCCN1CC(=C)C2 ; *CC[C@@]12CC(=C(F)F)CN1C[C@H](F)C2 ; *CC[C@@]12CC(=C)CN1C[C@H](F)C2 ; *CC[C@]12C[C@@H](F)CN1CC1(CC1)C2 ; *CC[C@@]12CCCN1C[C@H](F)C2 ; *CC[C@]12C[C@@H](F)CN1CCC2F]

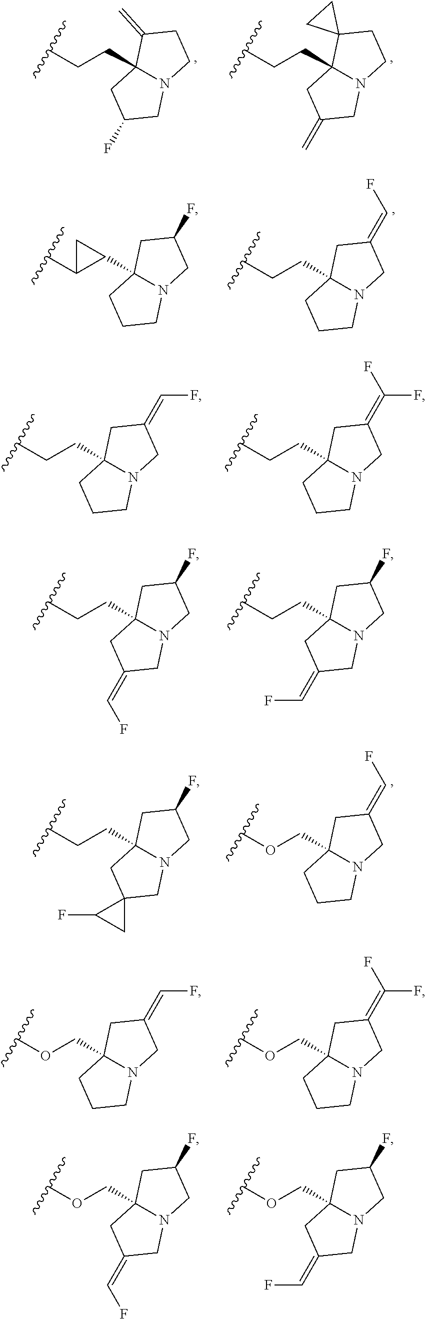

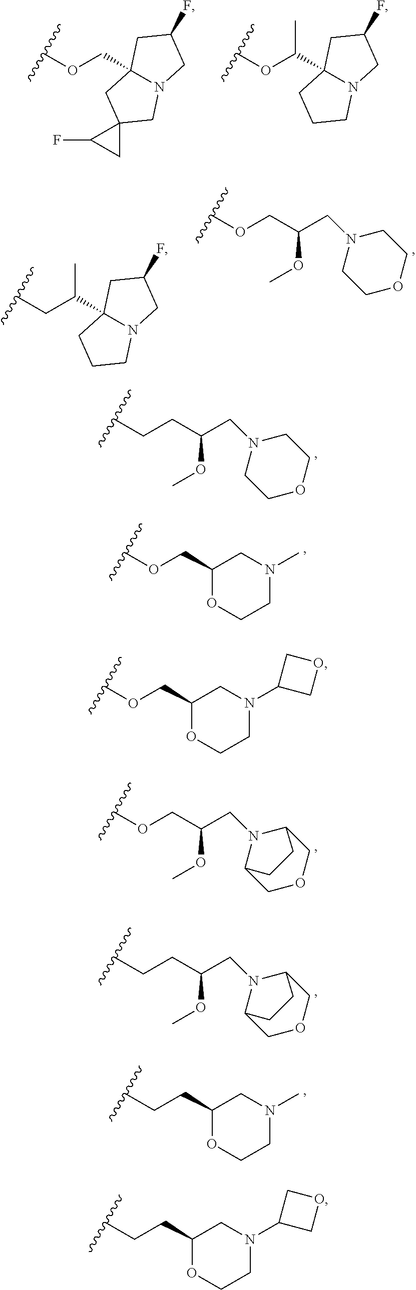

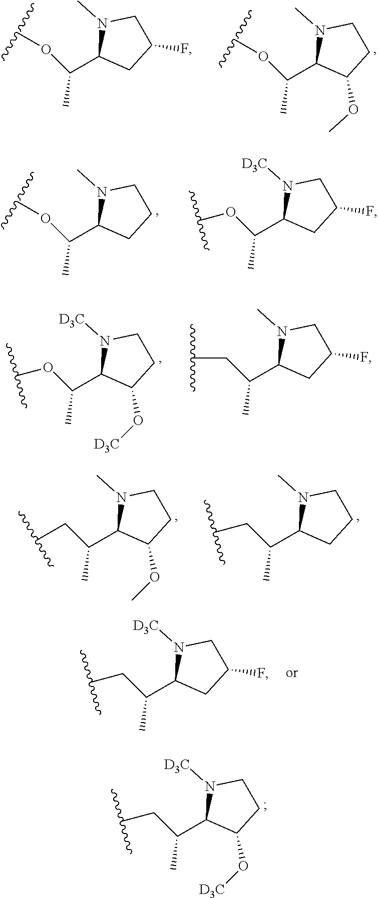

and
R₄ is:

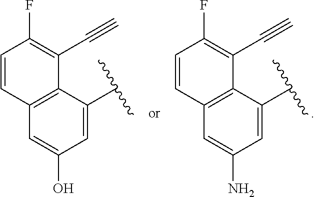 [SMILES: *c1cc(O)cc2ccc(F)c(C#C)c12 ; *c1cc(N)cc2ccc(F)c(C#C)c12]

In some embodiments, the compound is:

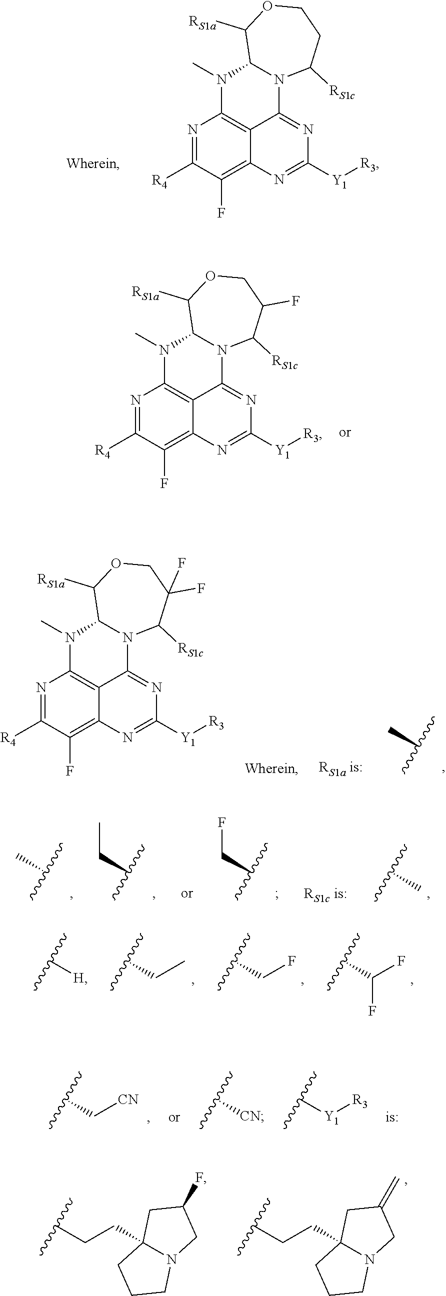

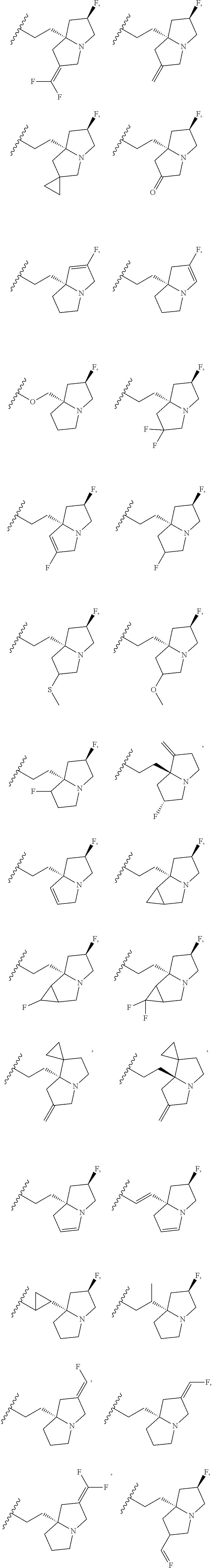

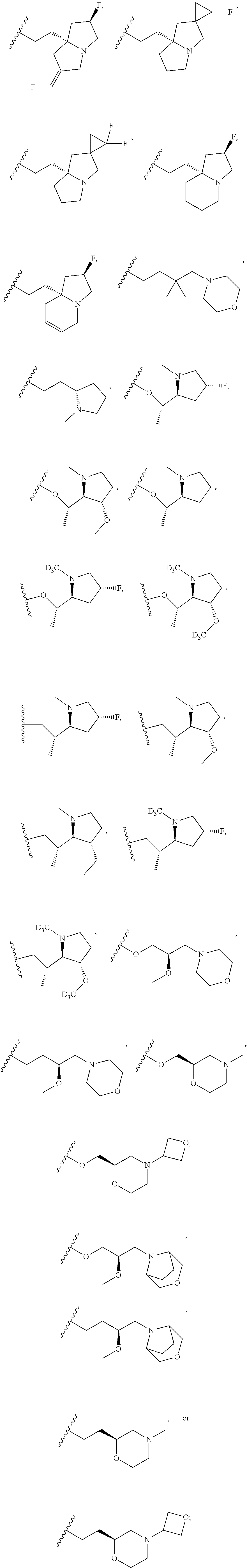

and
R₄ is:

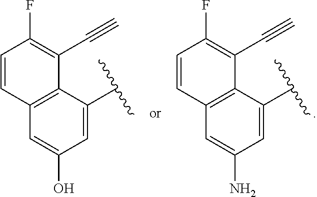 [SMILES: *c1cc(O)cc2ccc(F)c(C#C)c12 ; *c1cc(N)cc2ccc(F)c(C#C)c12]

In some embodiments, the compound is:

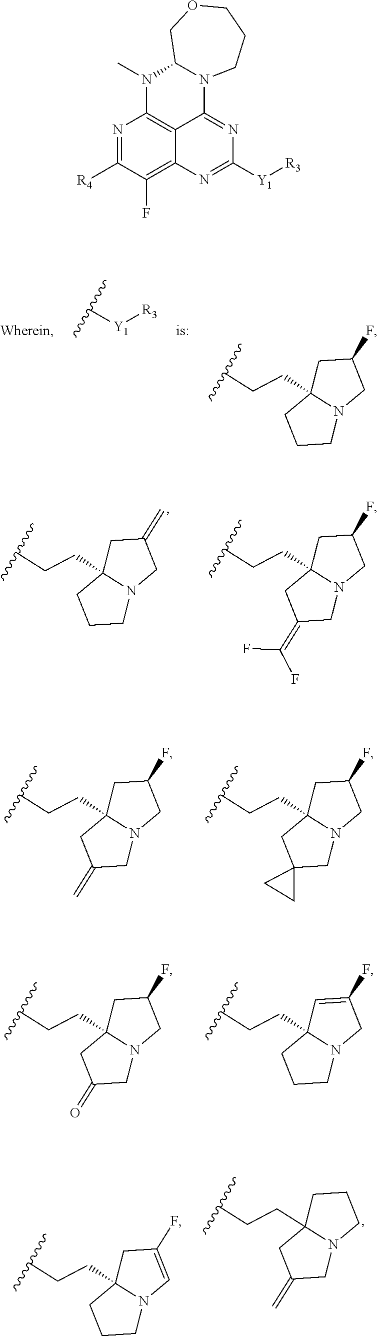

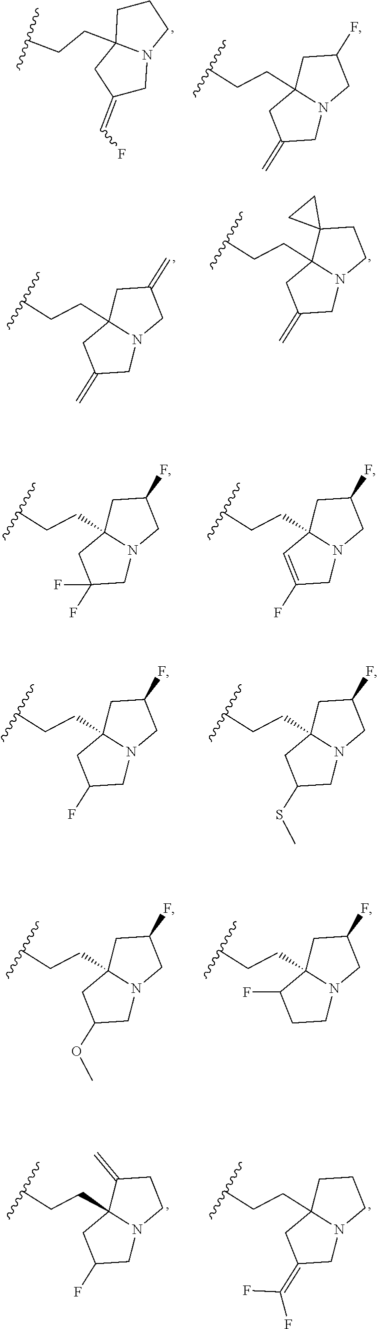

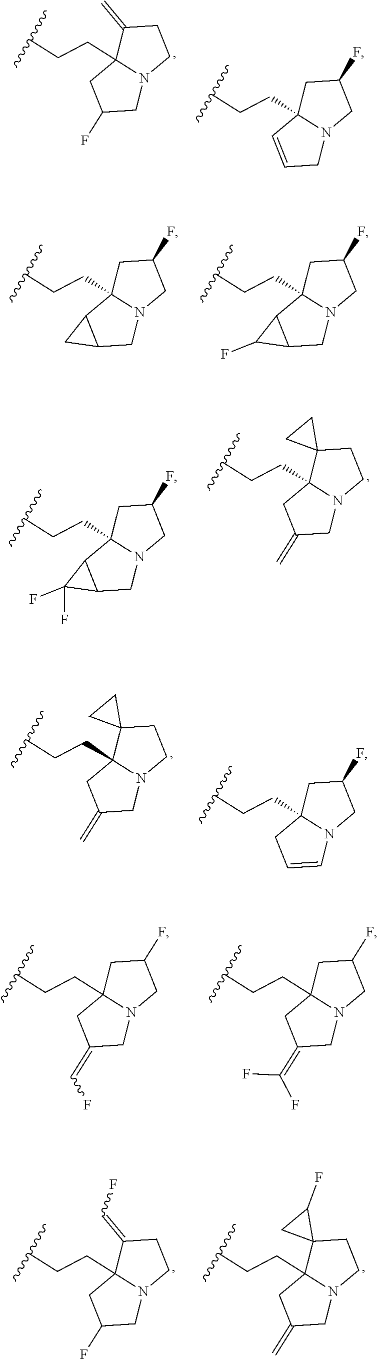

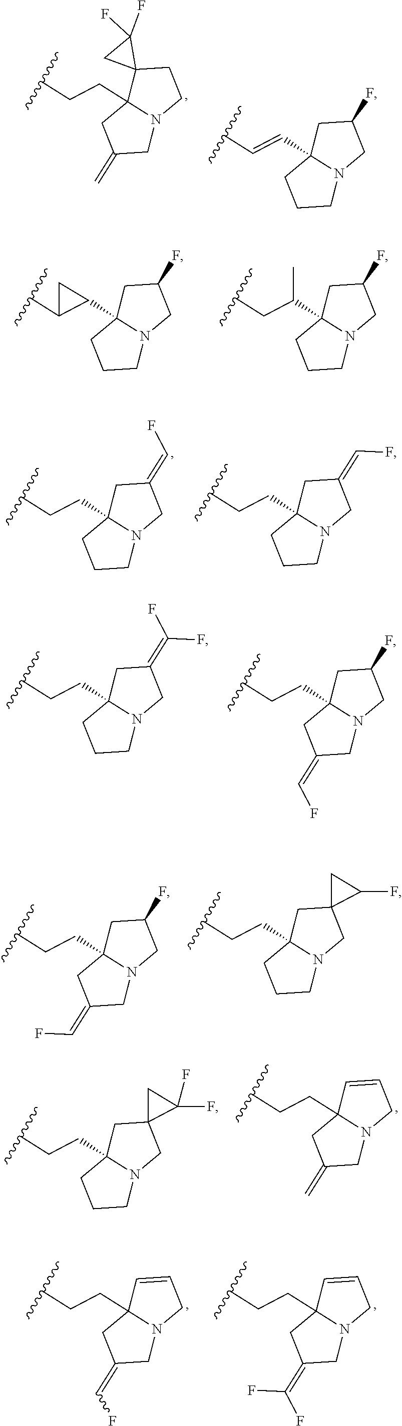

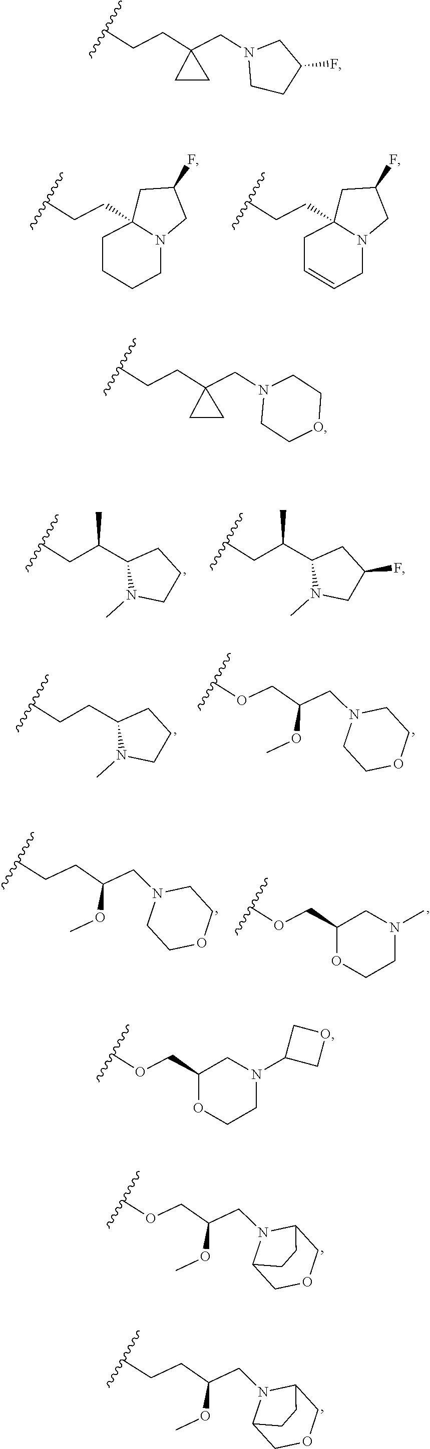

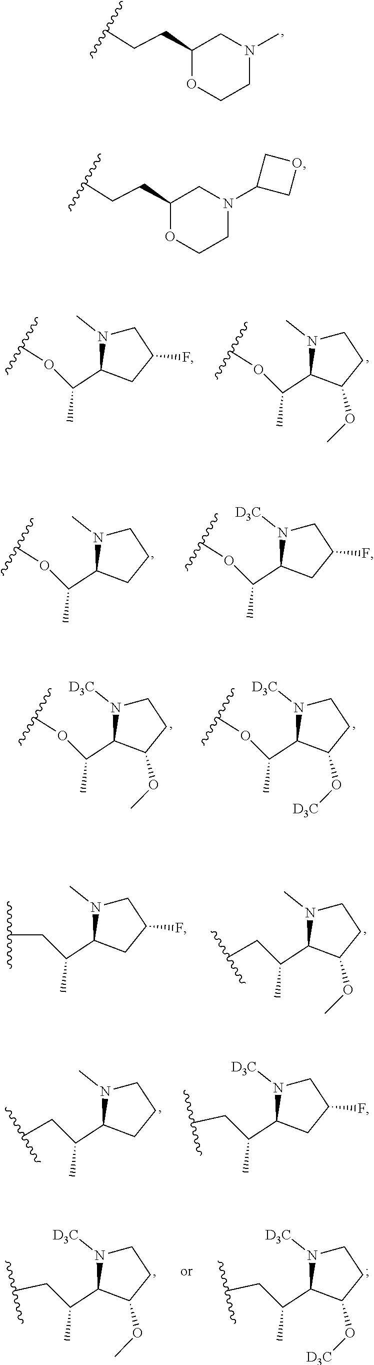

and
R₄ is:

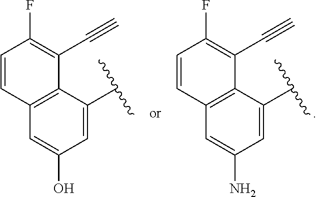 [SMILES: *c1cc(O)cc2ccc(F)c(C#C)c12 ; *c1cc(N)cc2ccc(F)c(C#C)c12]

In some embodiments, the compound is:

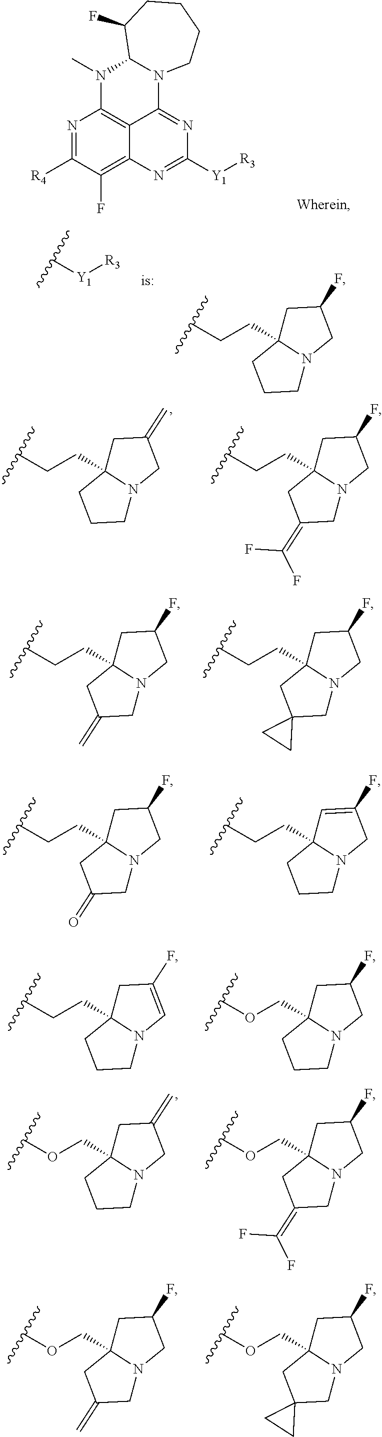

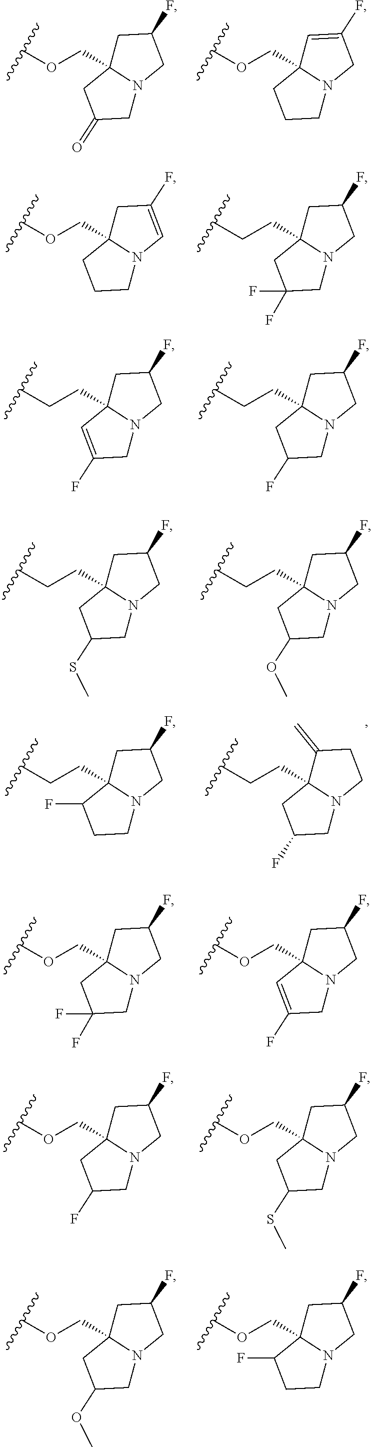

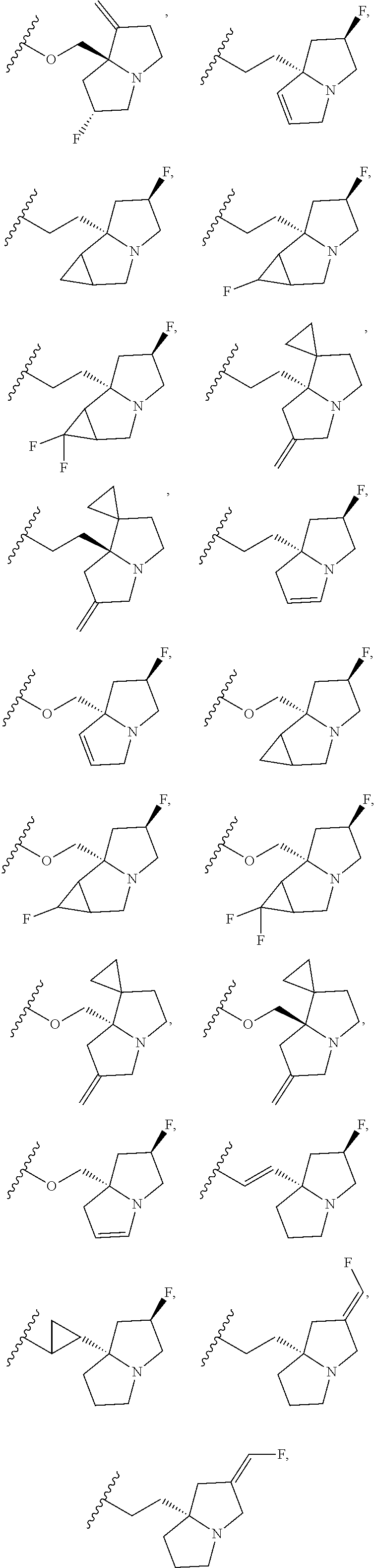

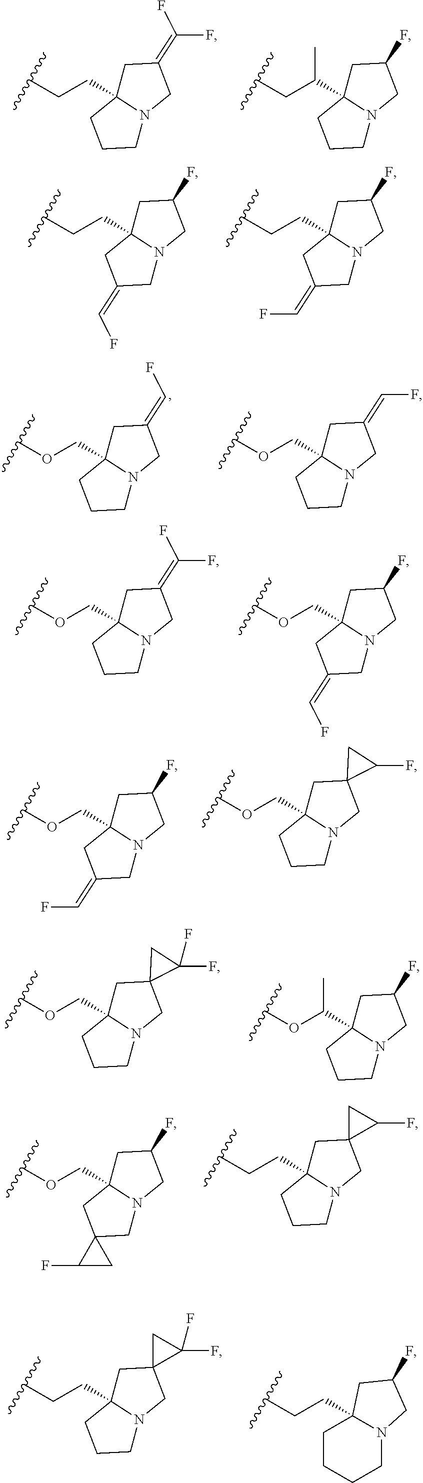

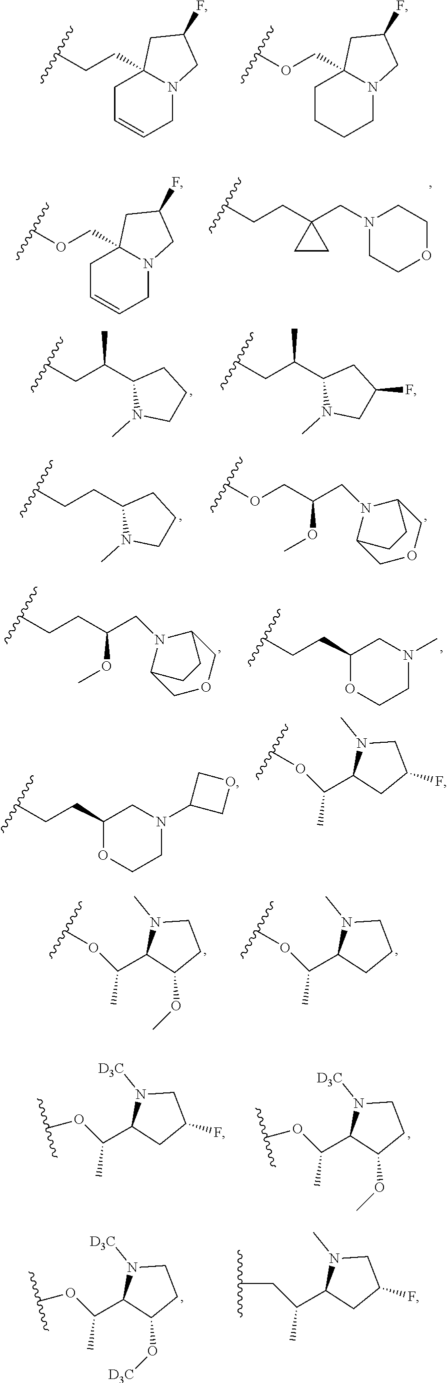

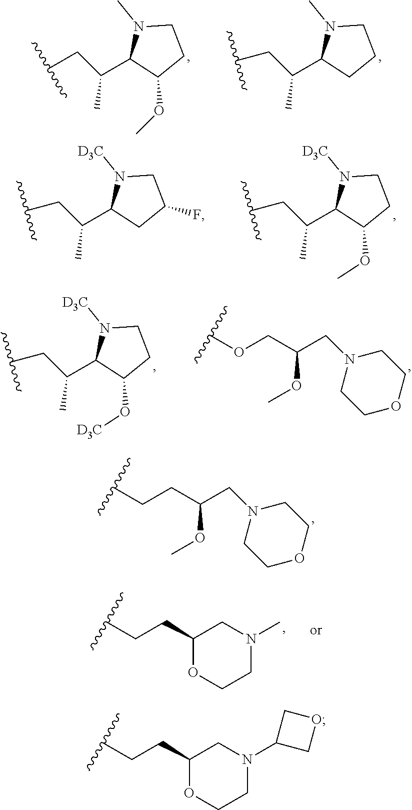

and
R₄ is:

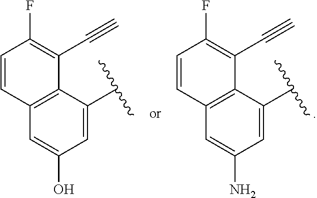 [SMILES: *c1cc(O)cc2ccc(F)c(C#C)c12 ; *c1cc(N)cc2ccc(F)c(C#C)c12]

In some embodiments, the compound is:

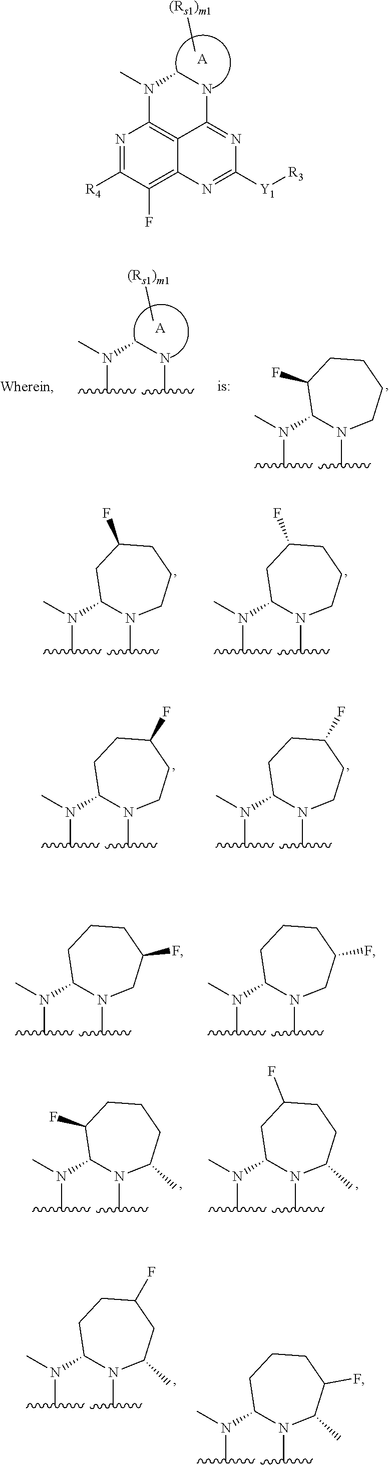

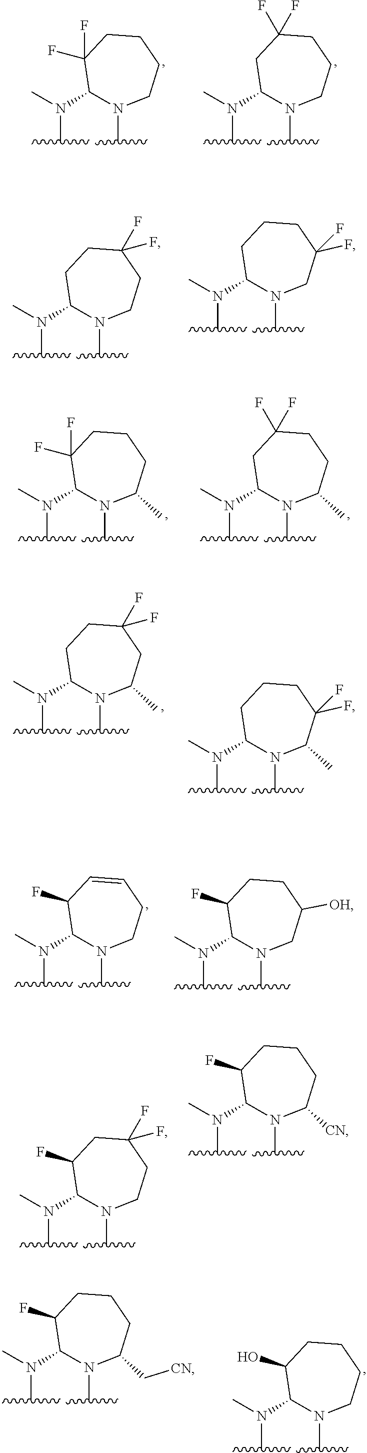

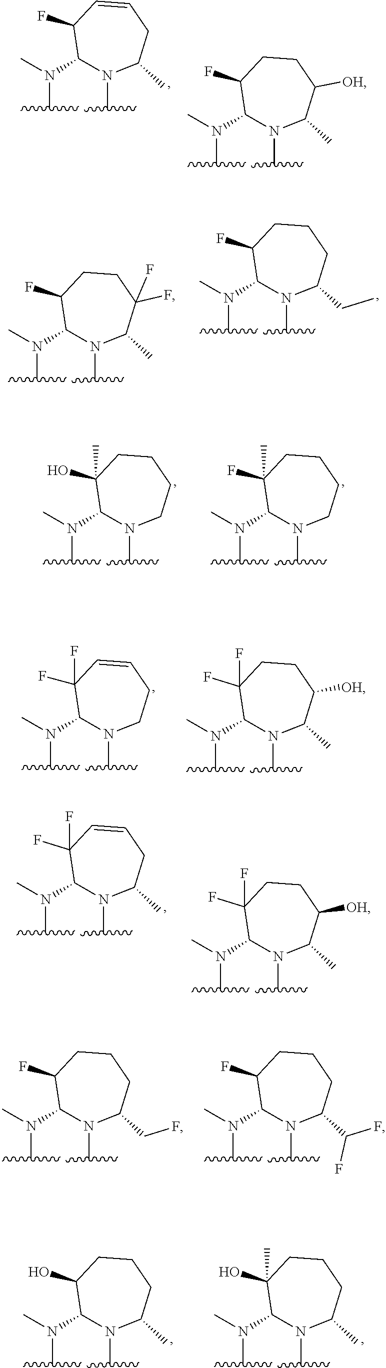

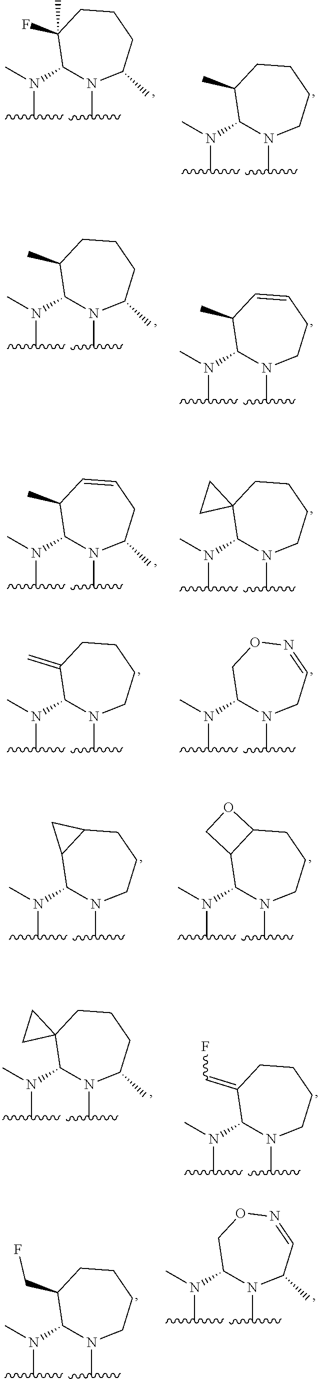

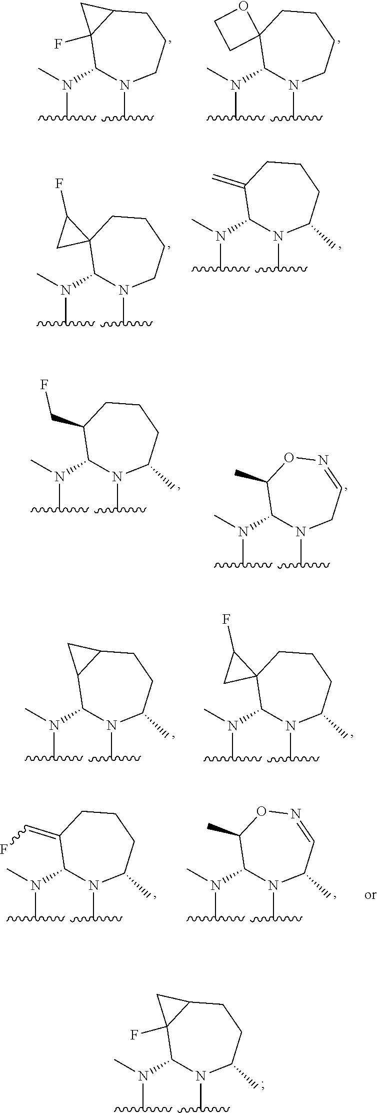

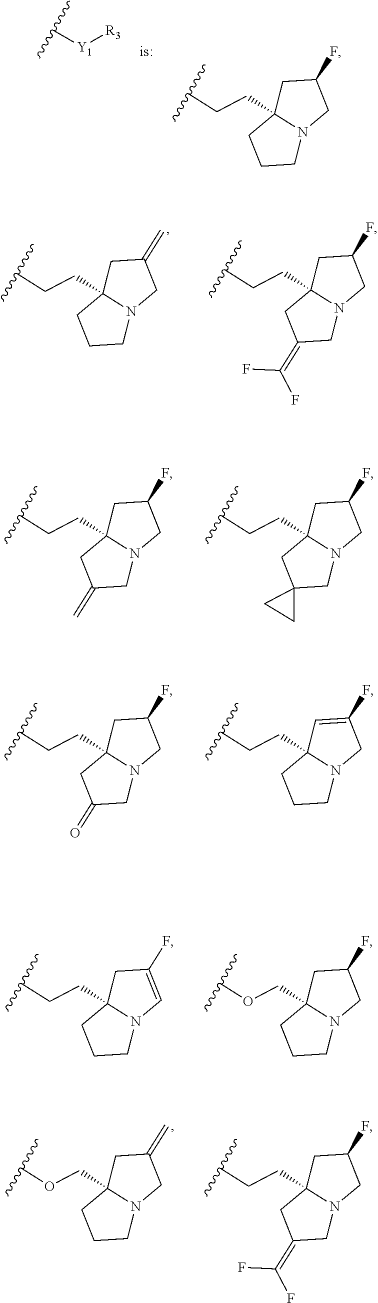

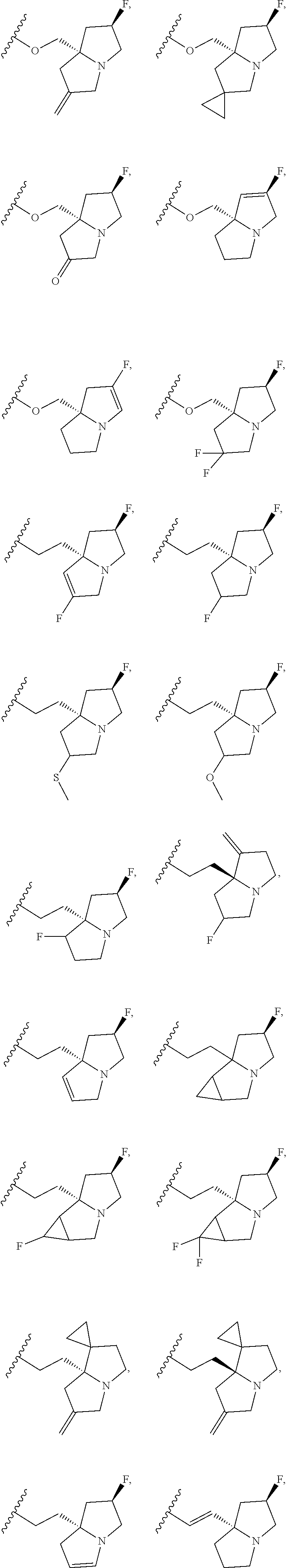

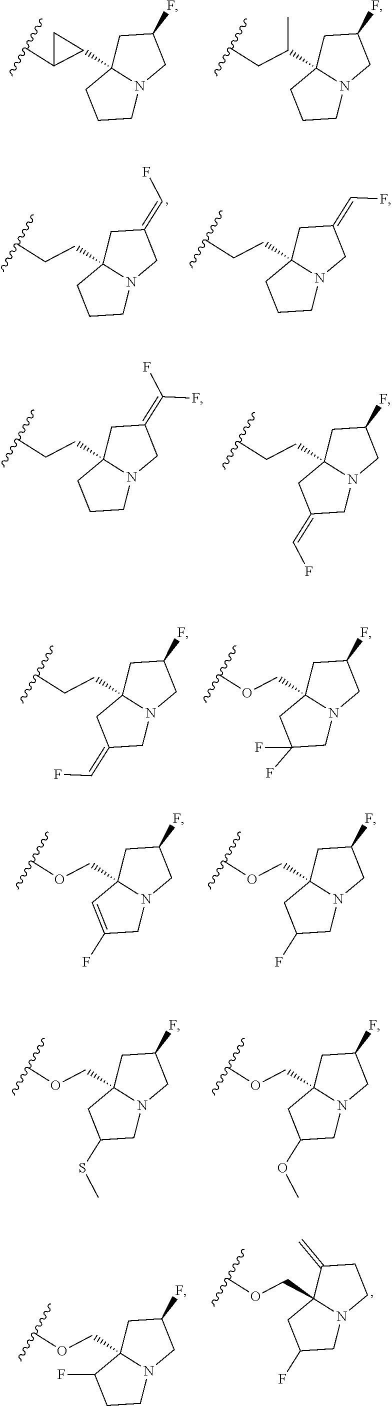

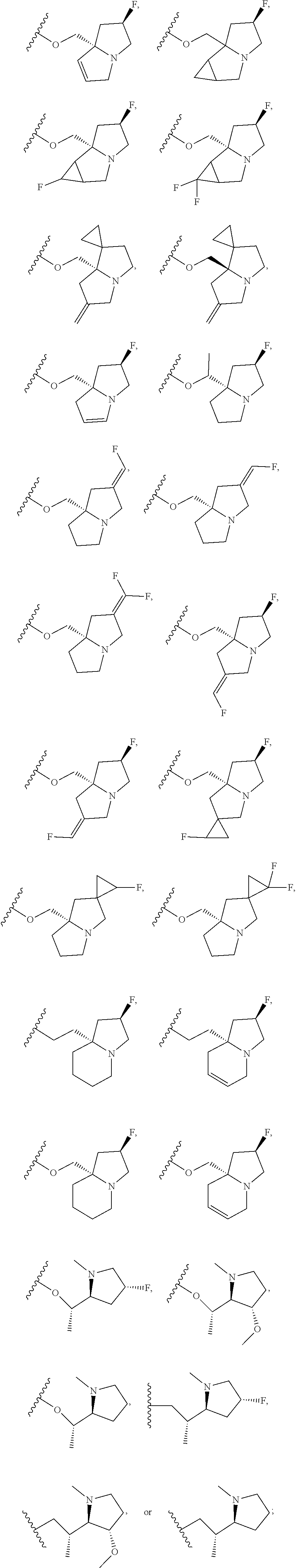

and
R₄ is:

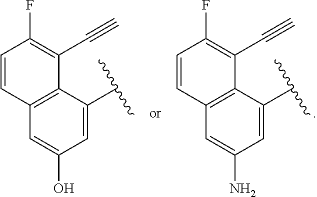 [SMILES: *c1cc(O)cc2ccc(F)c(C#C)c12 ; *c1cc(N)cc2ccc(F)c(C#C)c12]

In some embodiments, the compound is:

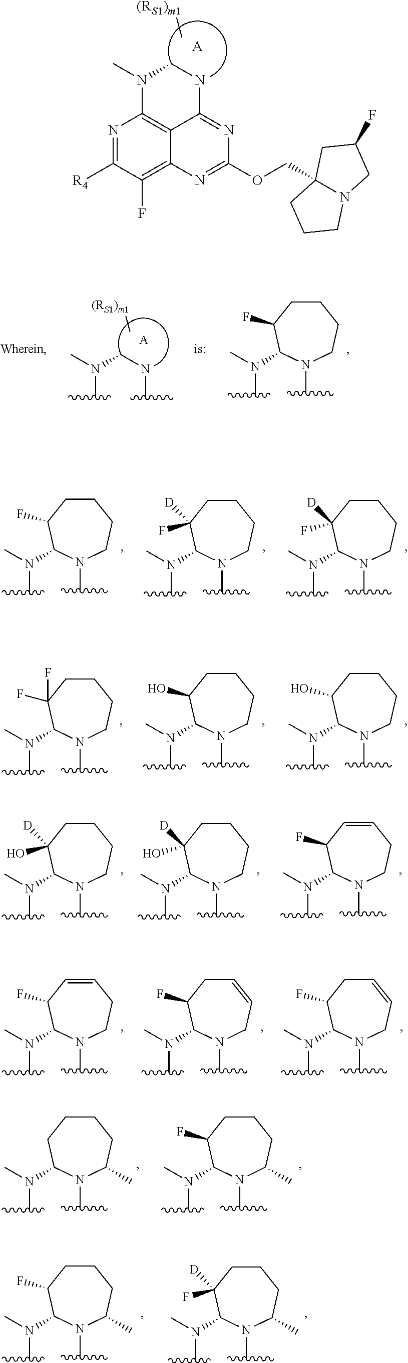

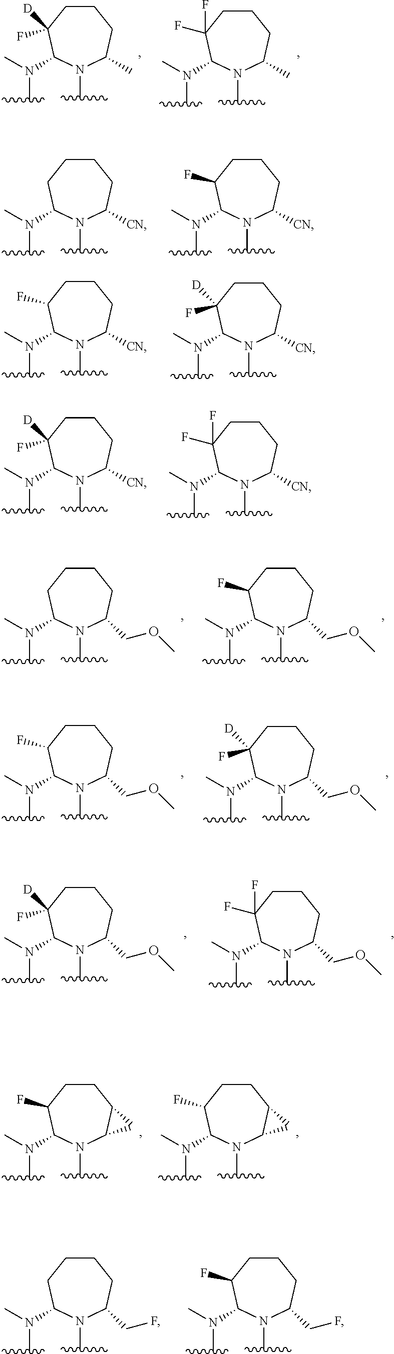

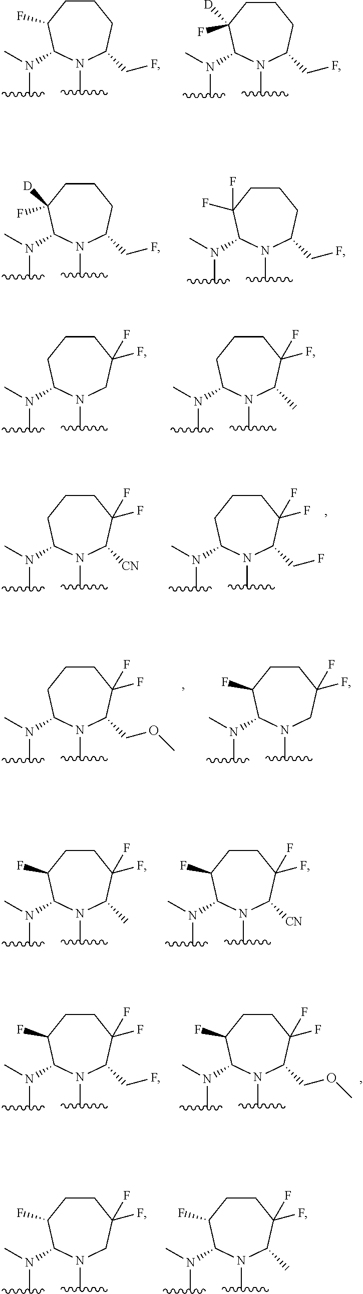

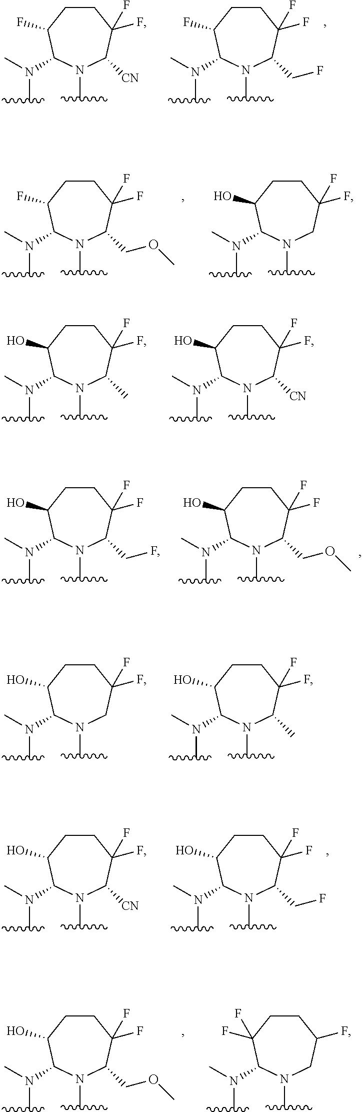

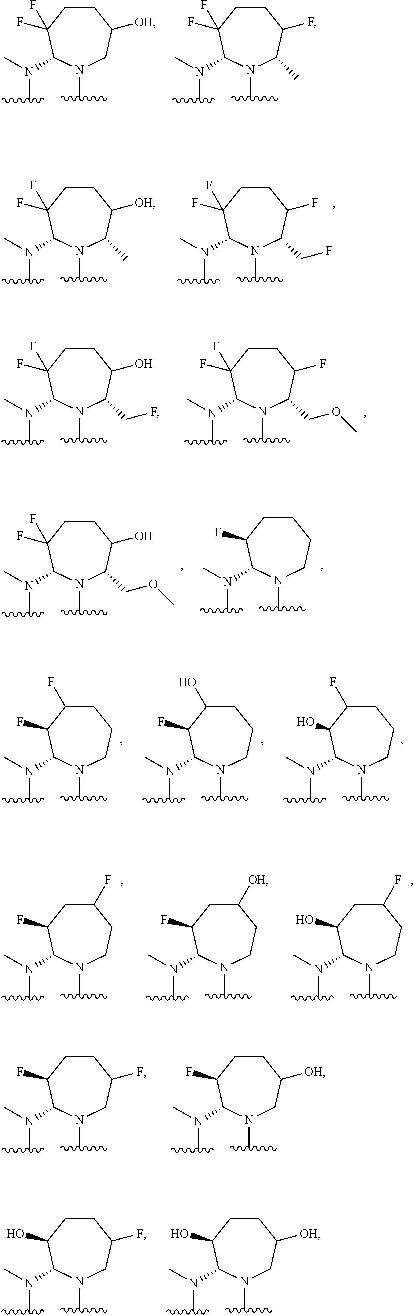

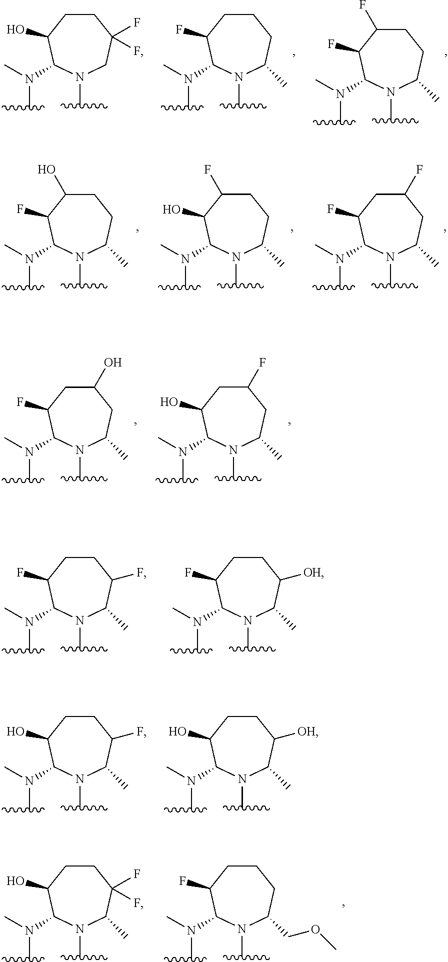

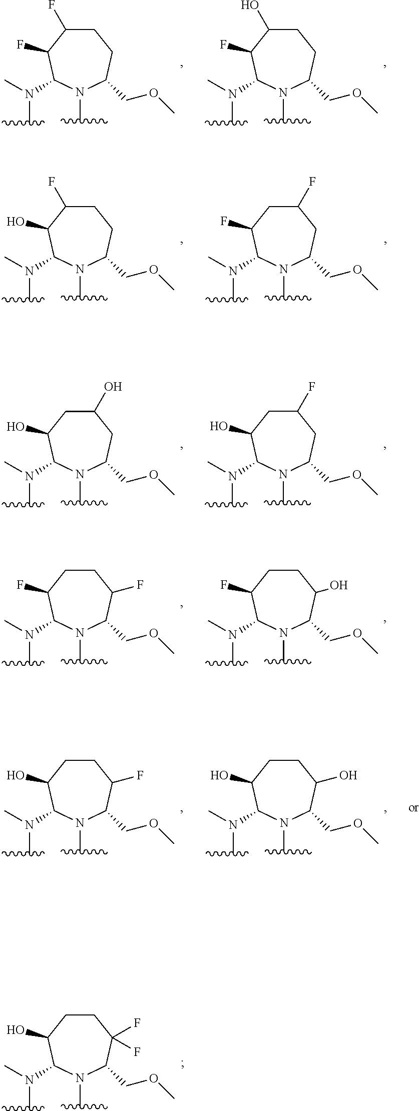

and
R₄ is:

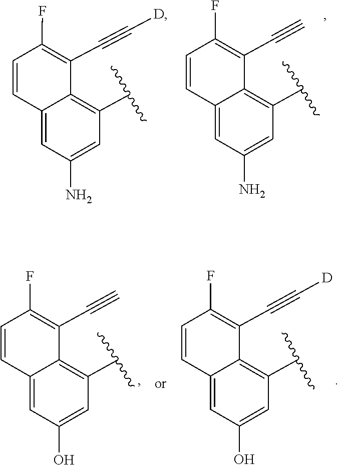

In some embodiments, the compound is:

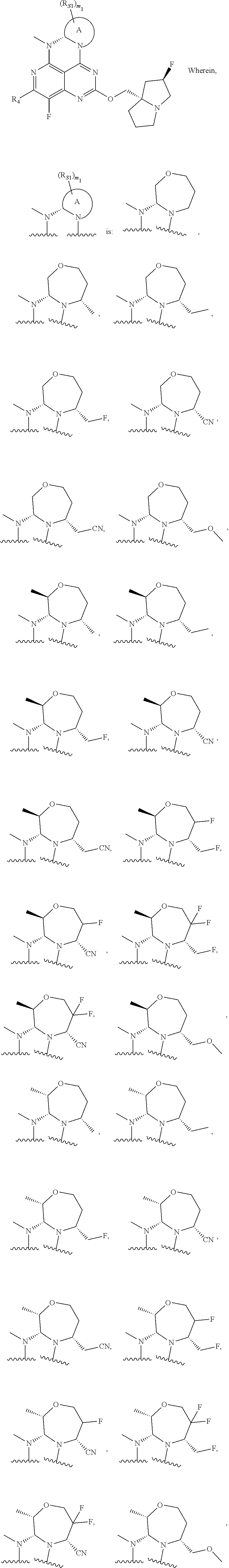

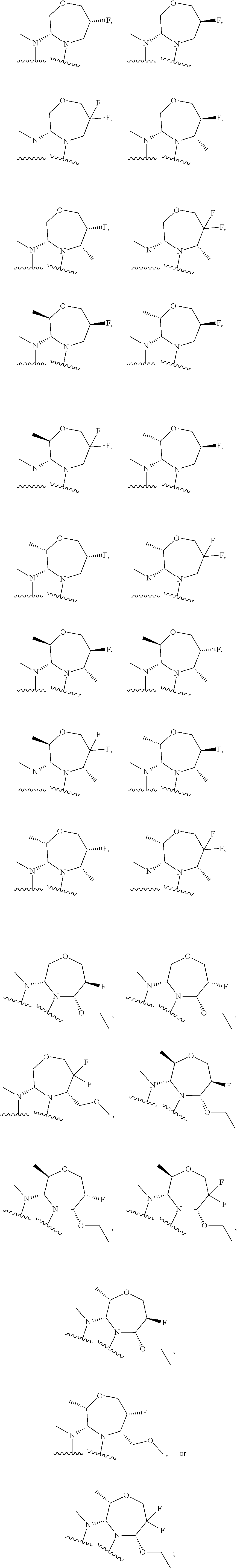

and
R₄ is:

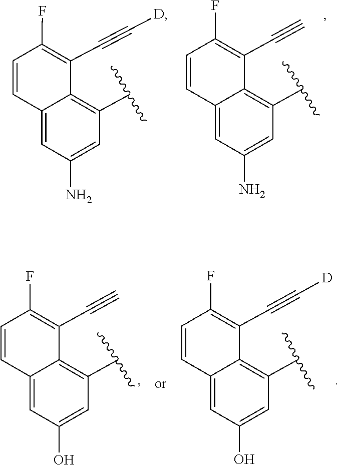

In some embodiments, the compound is:

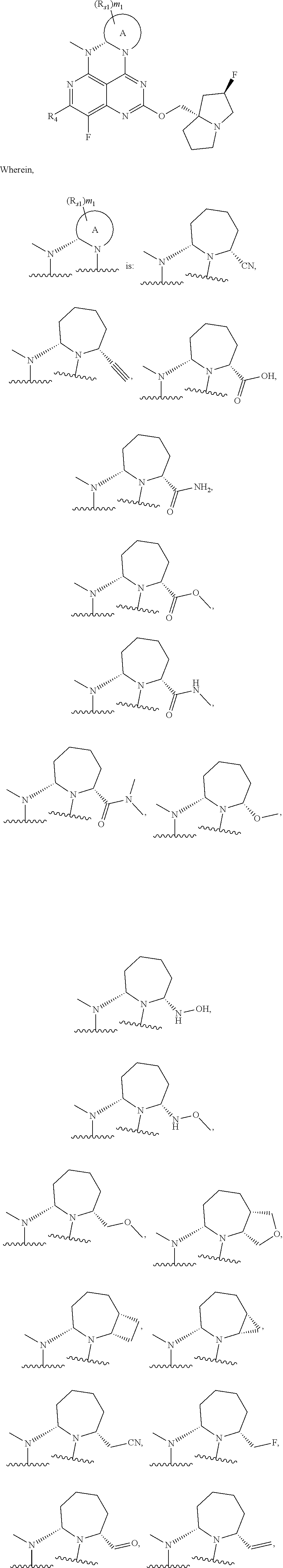

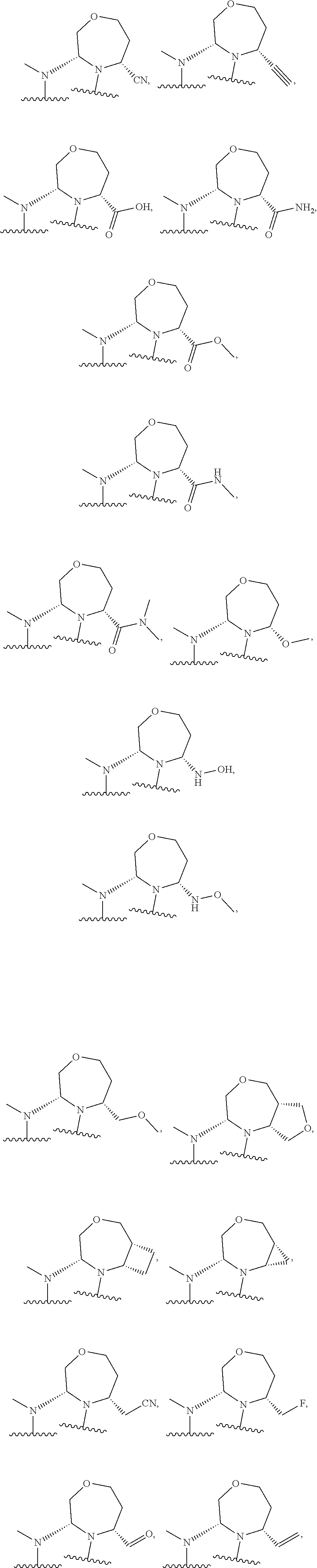

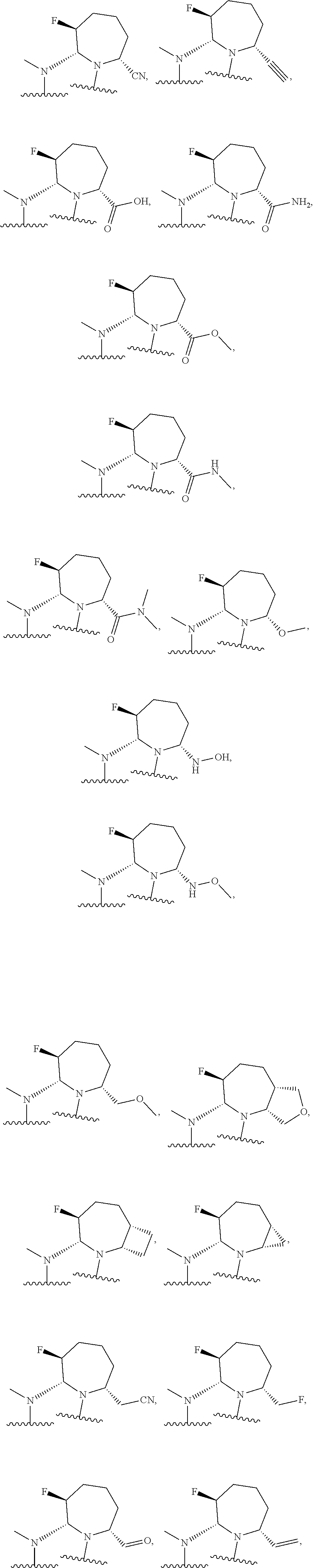

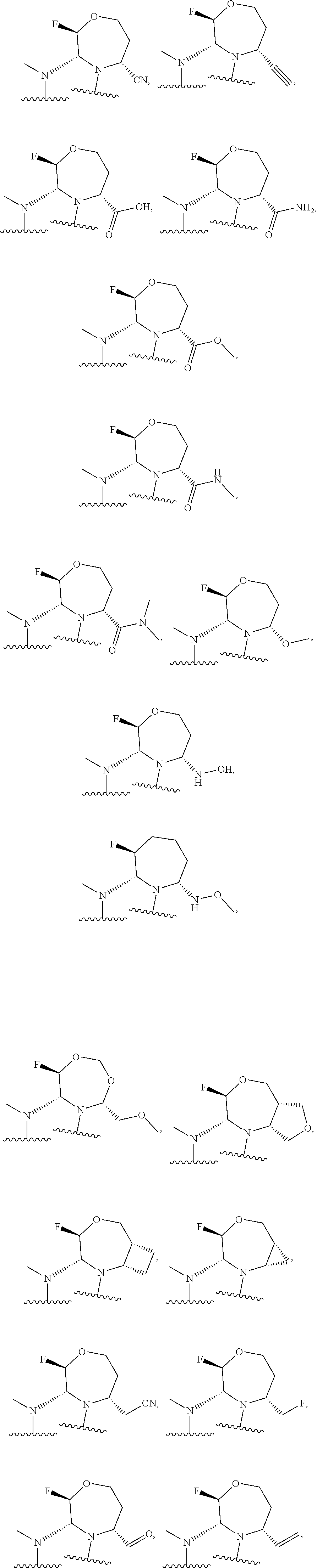

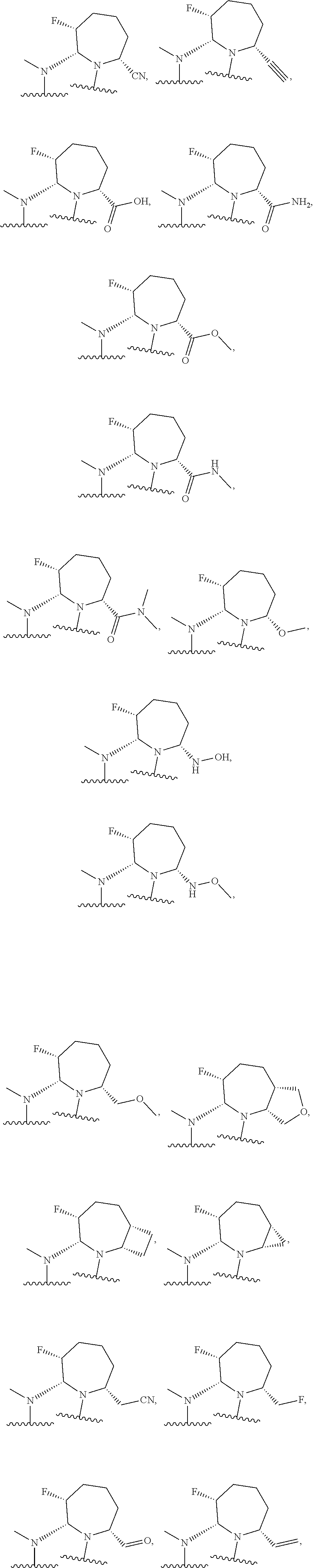

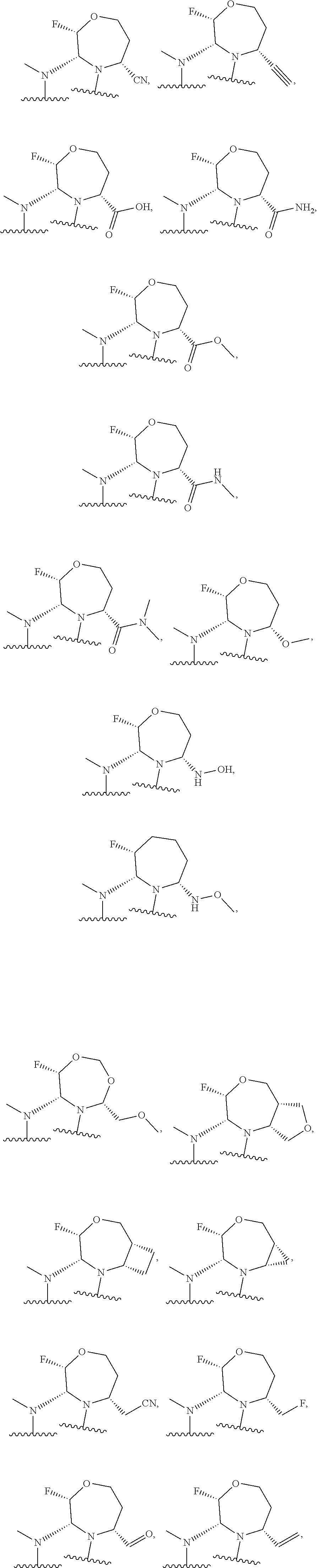

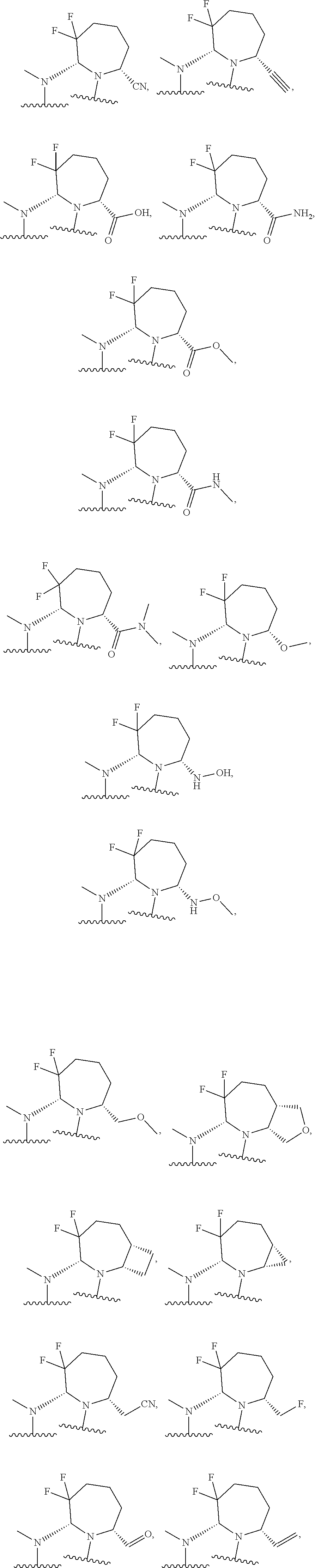

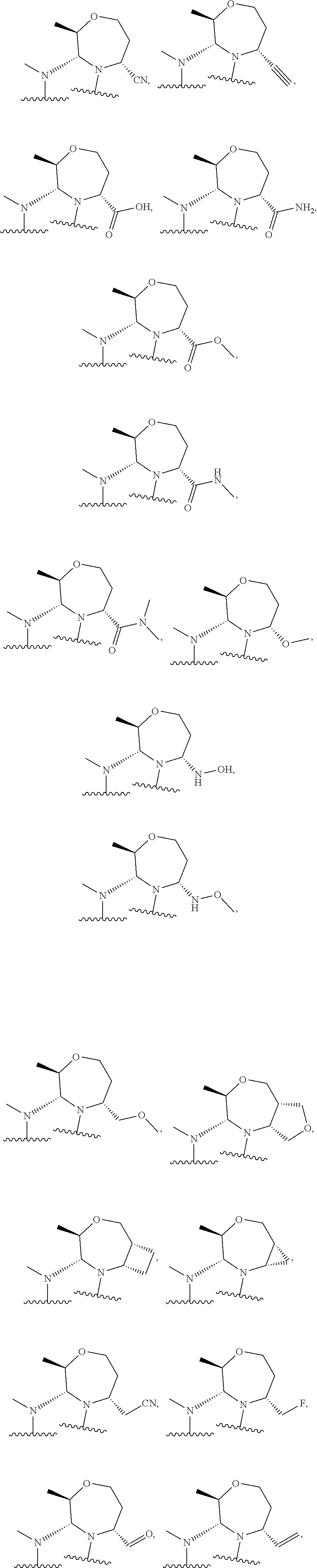

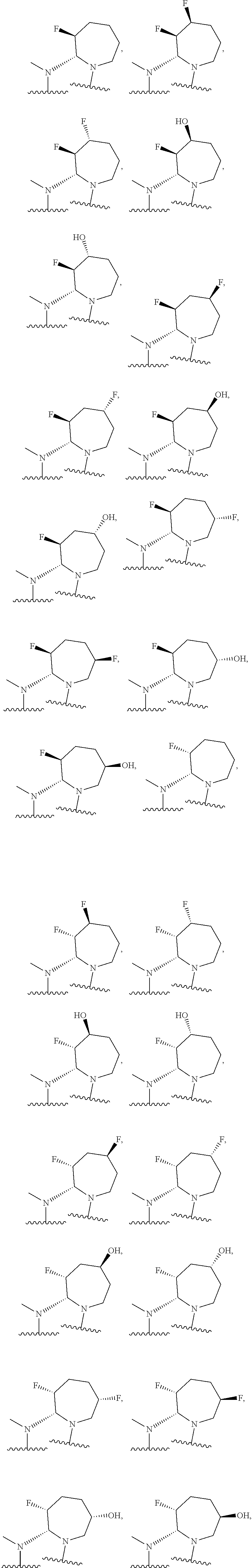

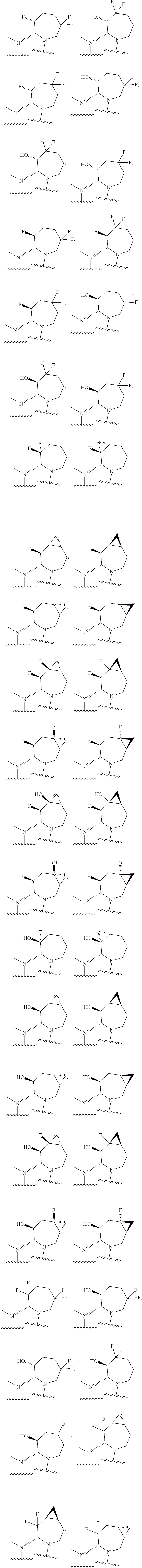

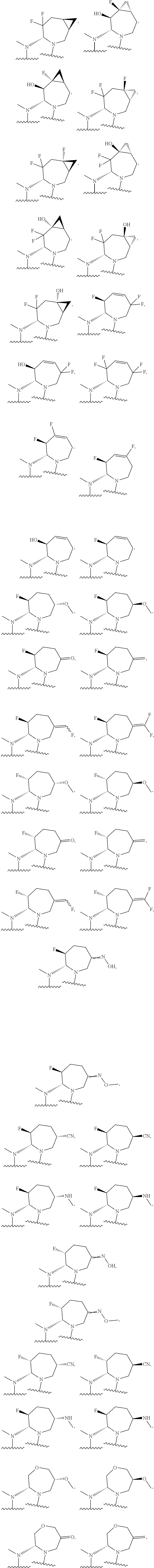

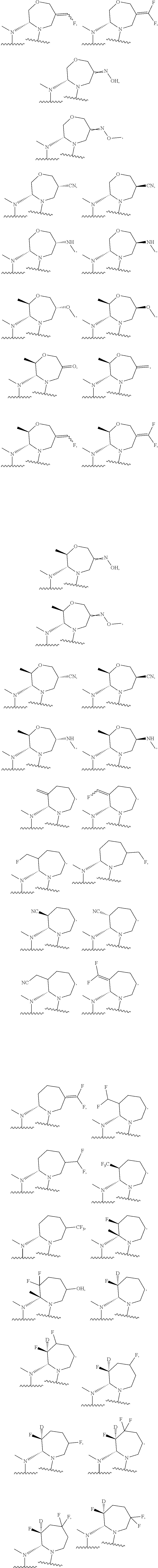

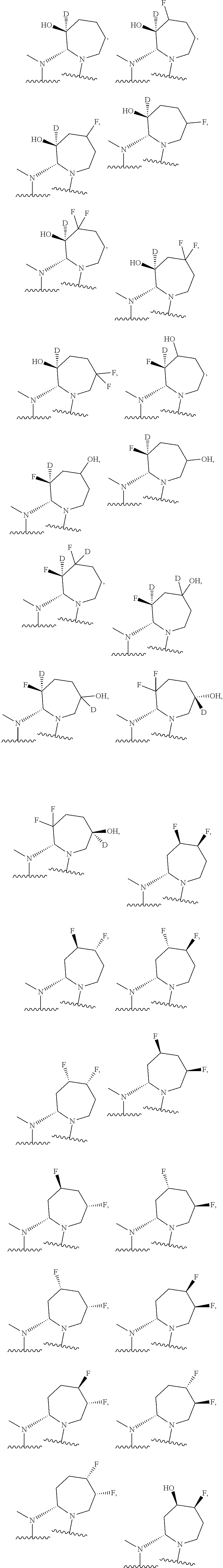

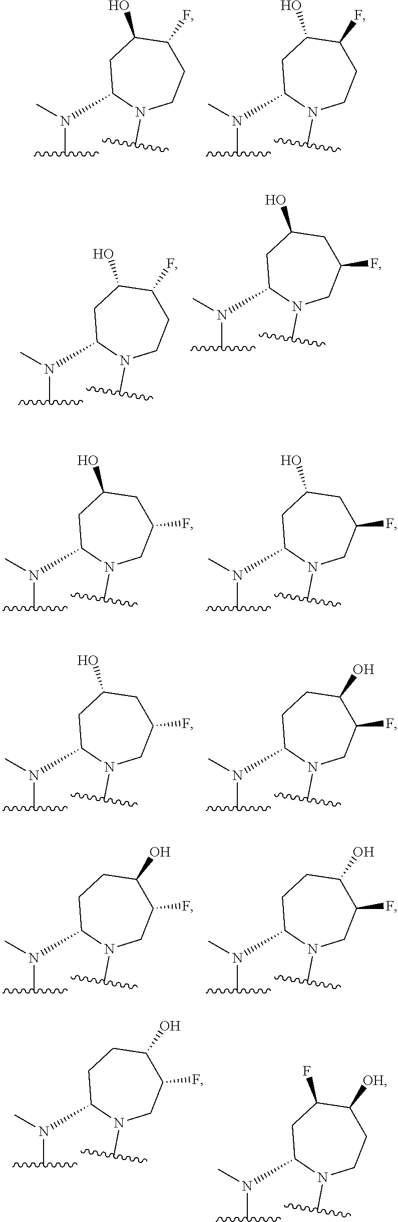

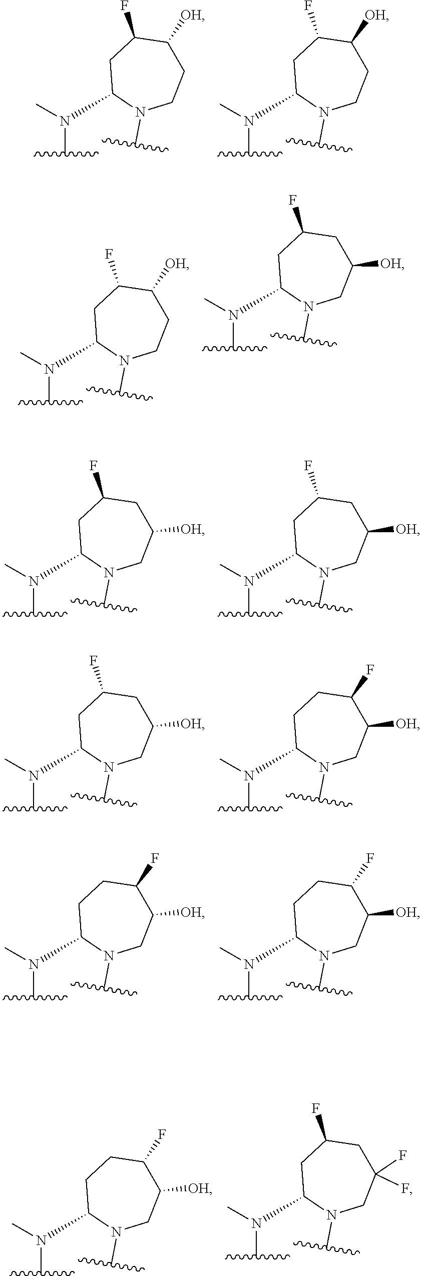

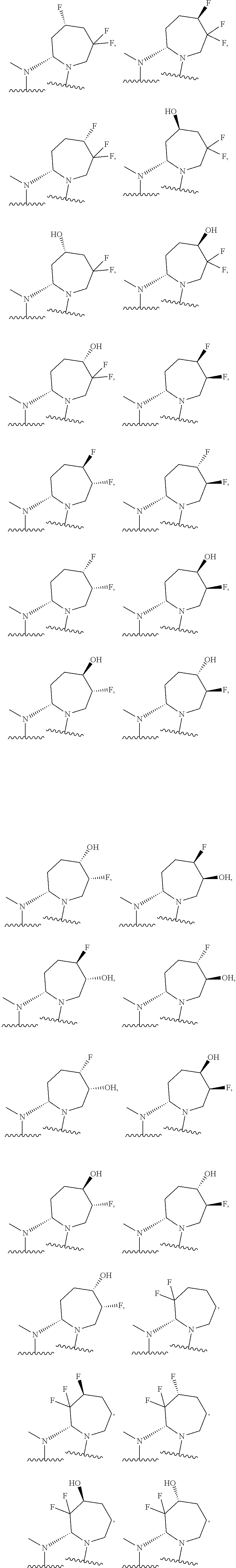

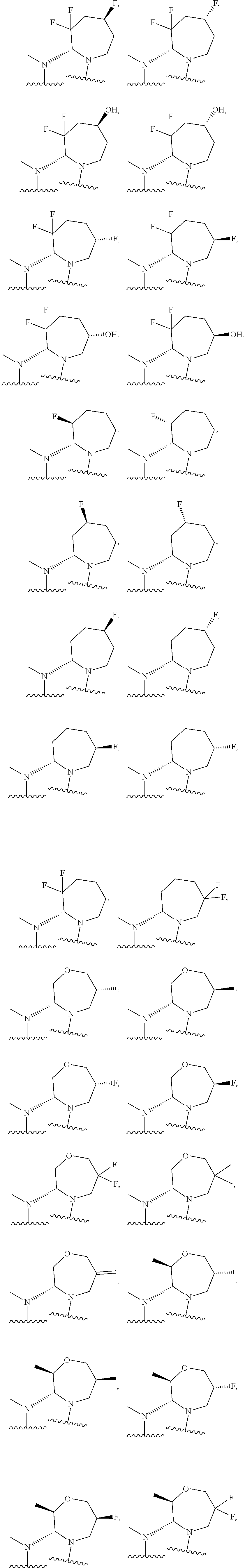

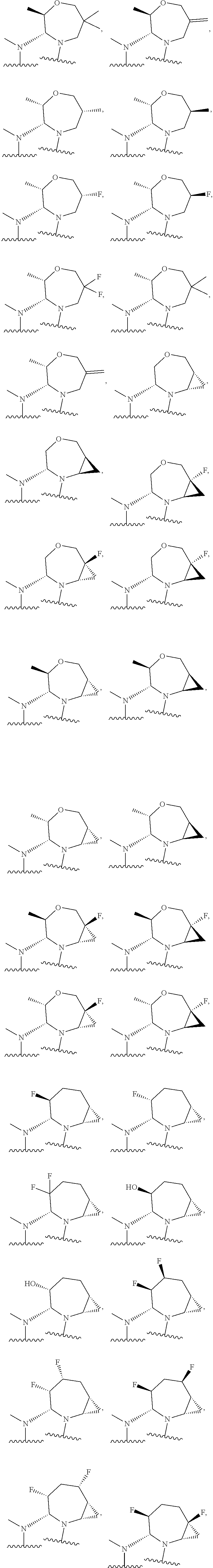

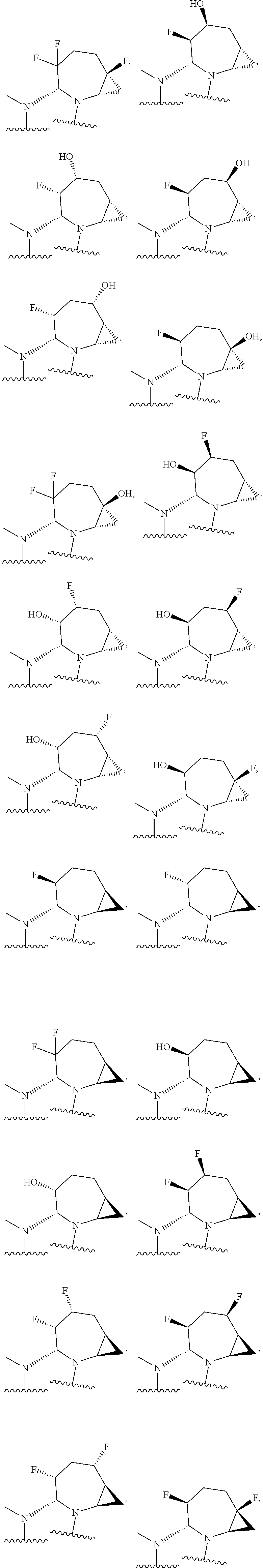

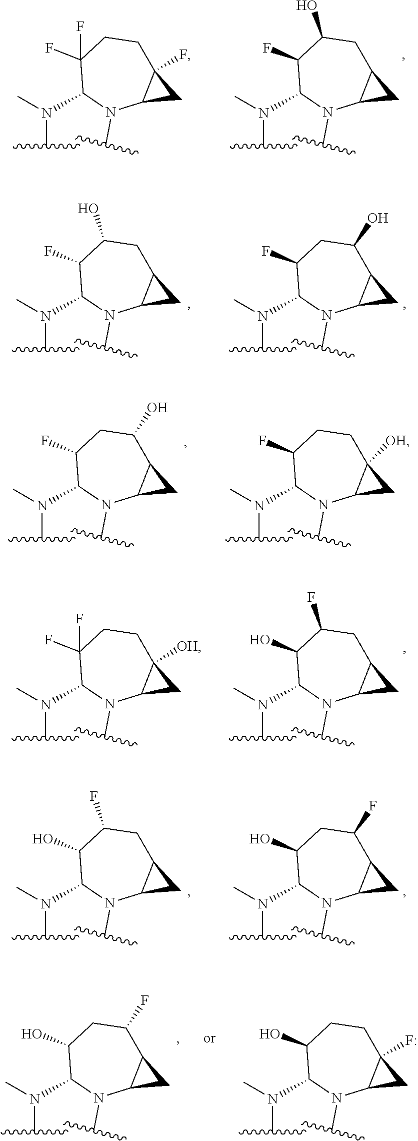

and
R₄ is:

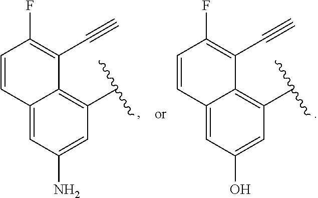 [SMILES: *c1cc(N)cc2ccc(F)c(C#C)c12 ; *c1cc(O)cc2ccc(F)c(C#C)c12]

In some embodiments, the compound is:

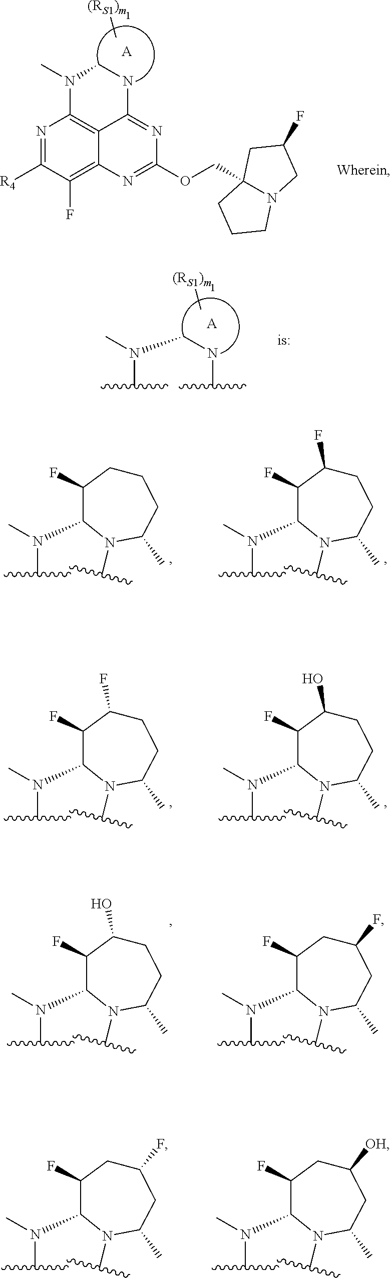

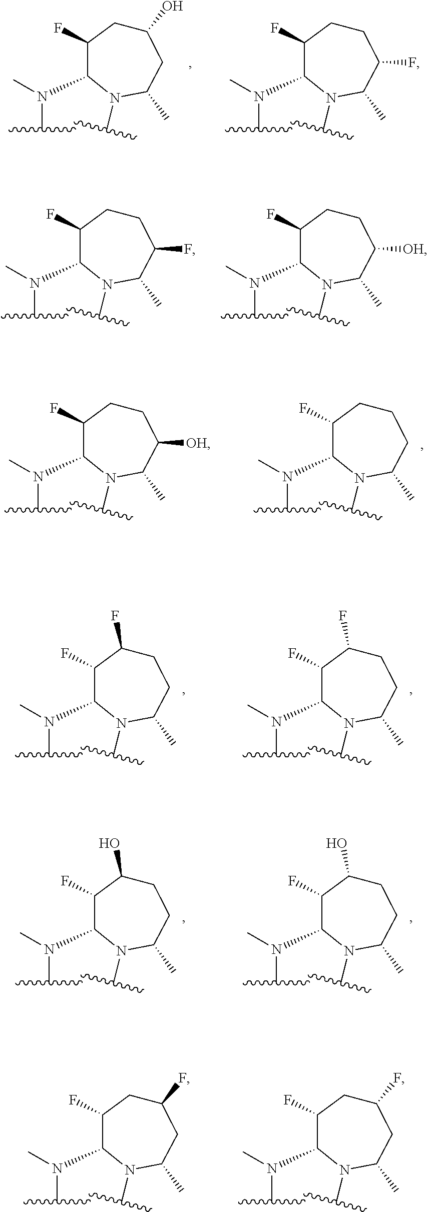

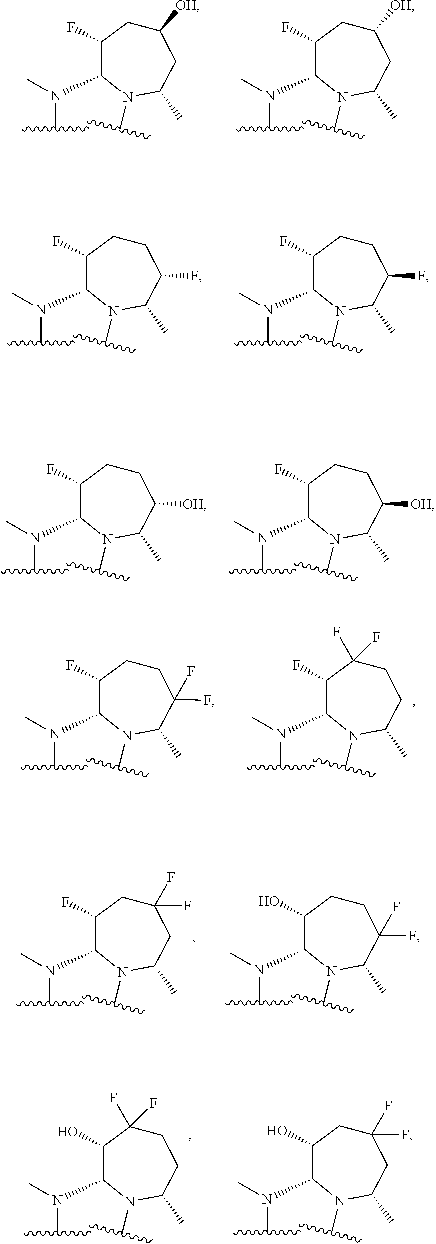

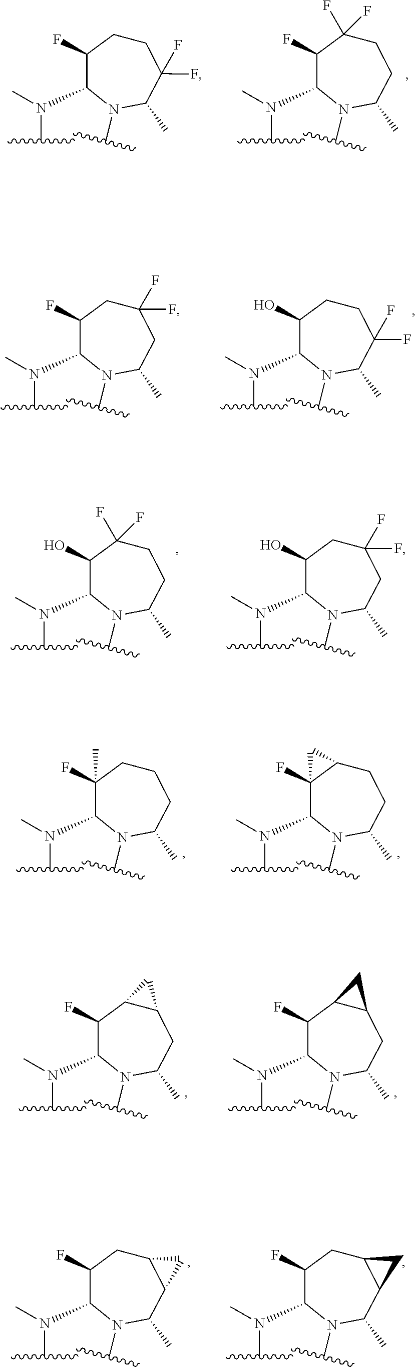

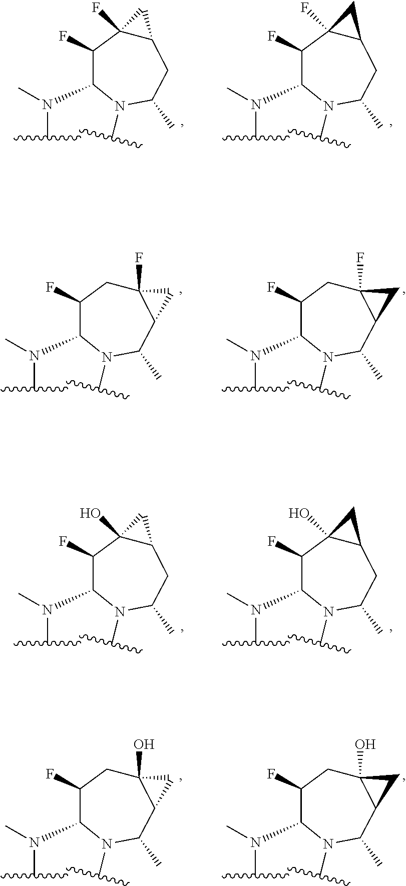

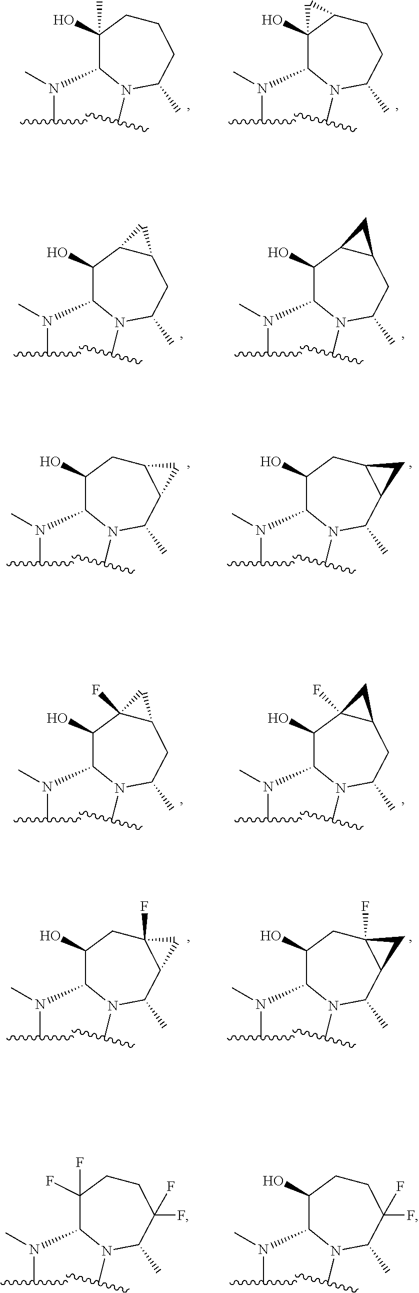

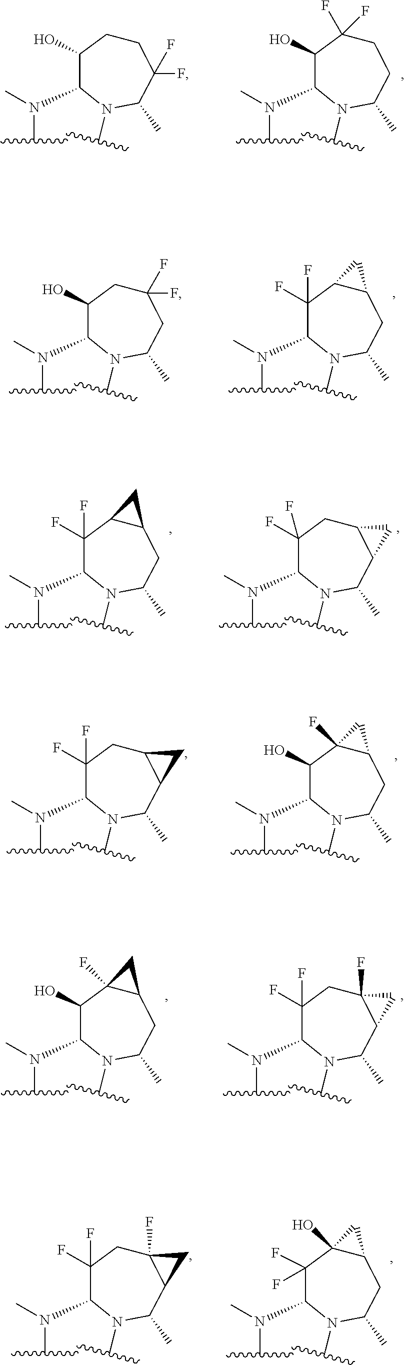

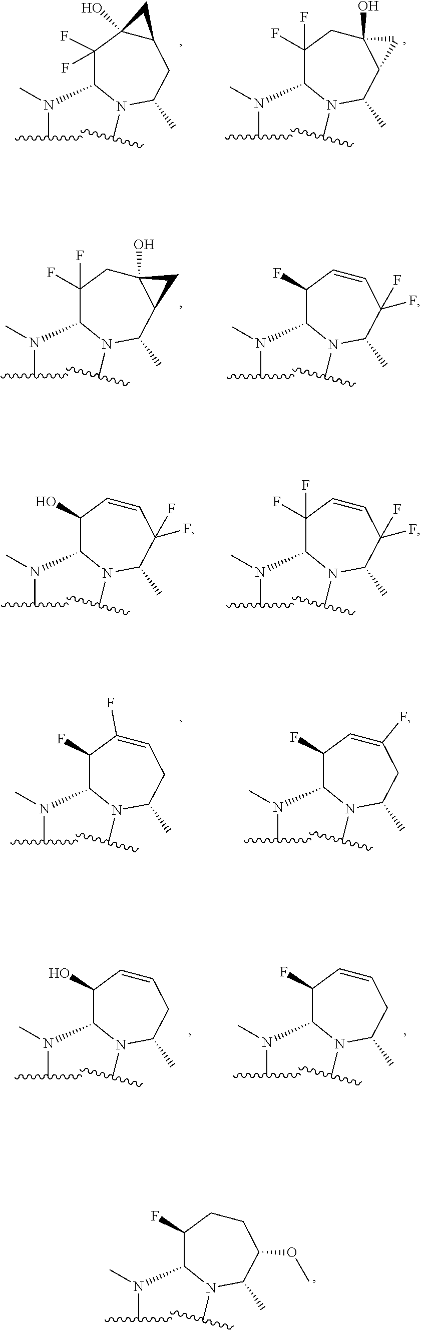

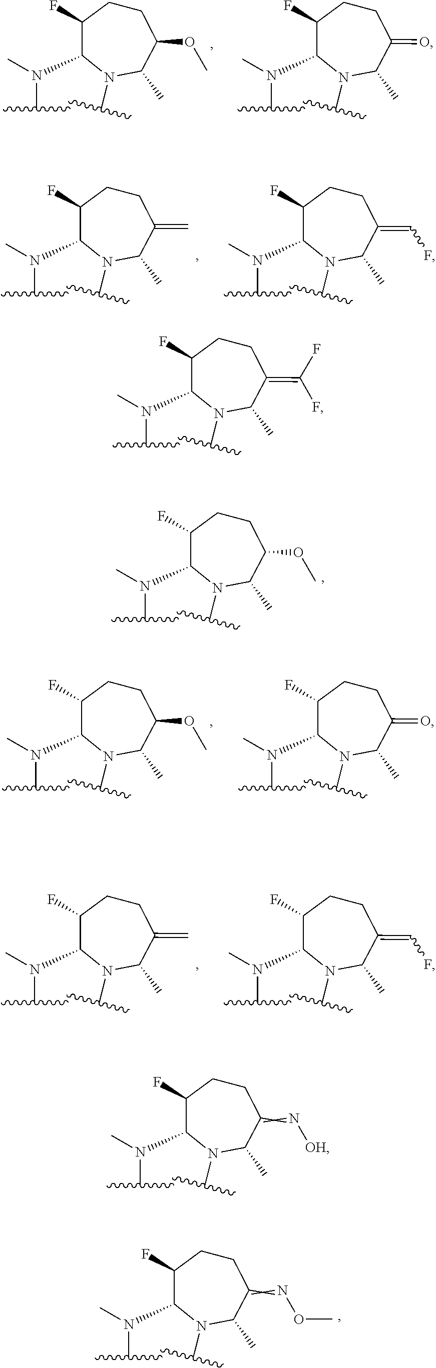

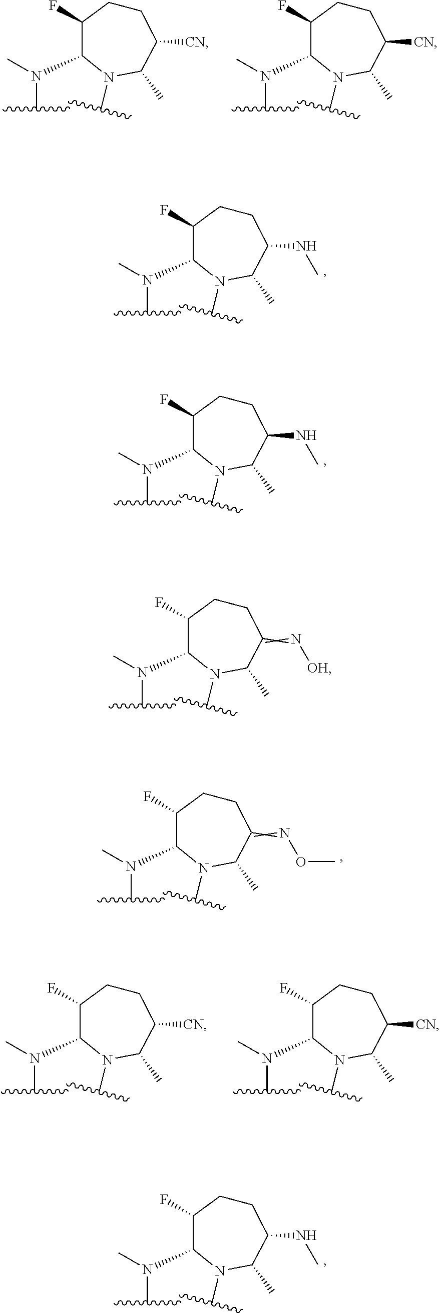

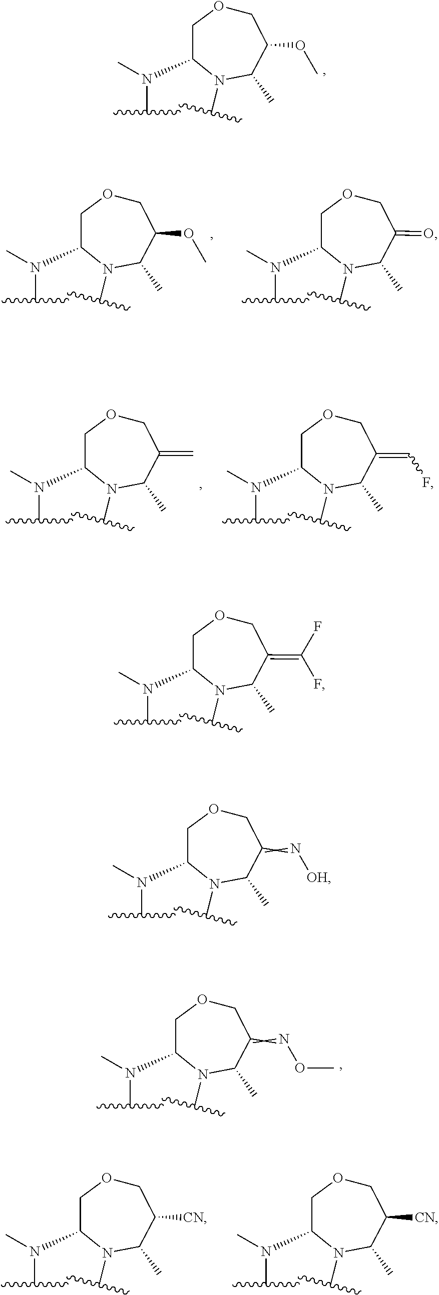

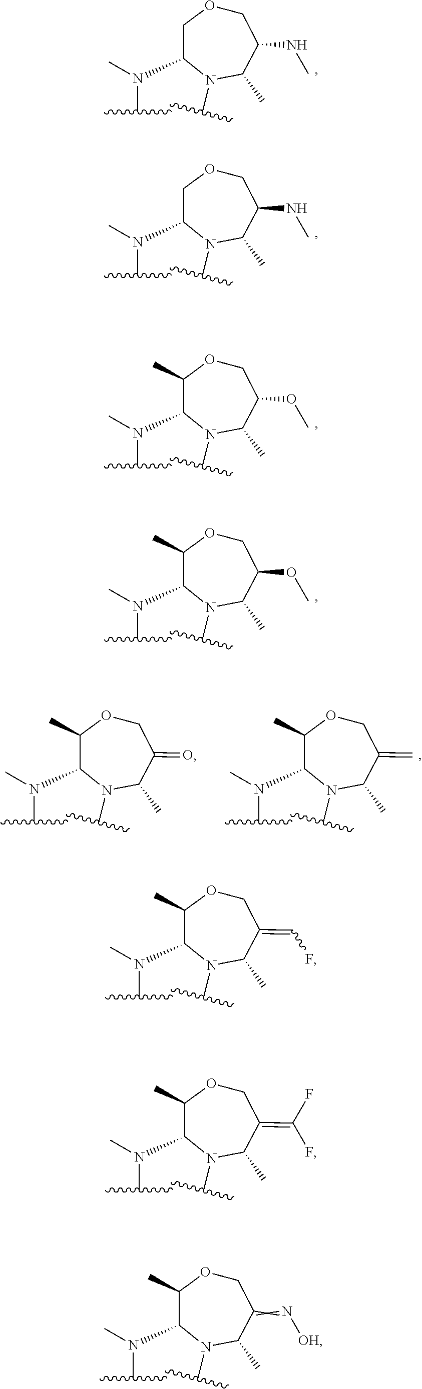

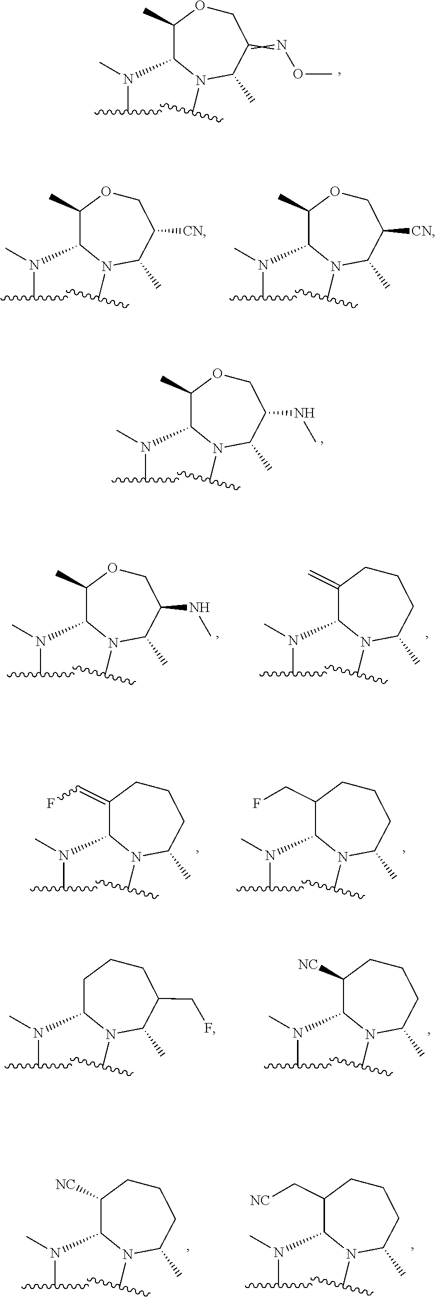

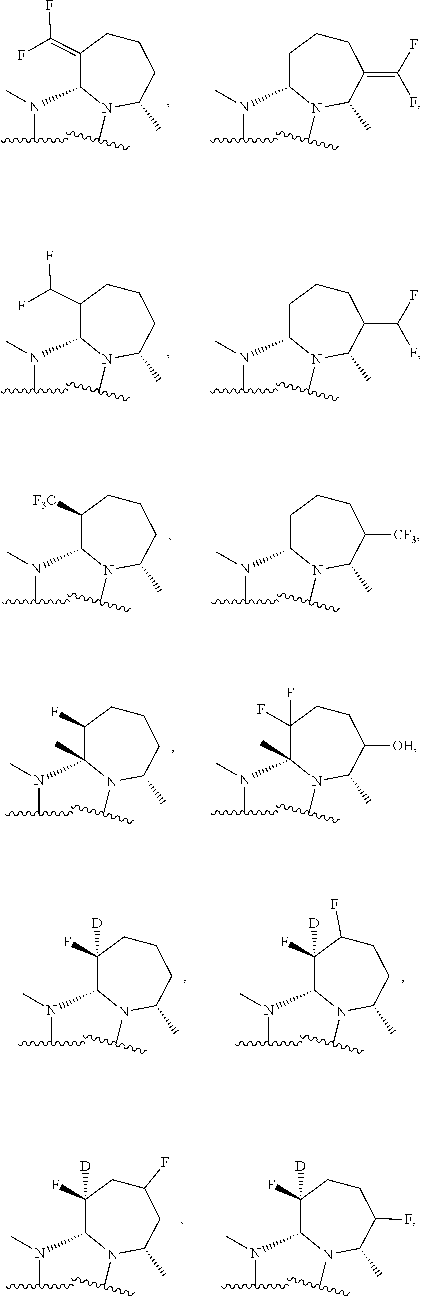

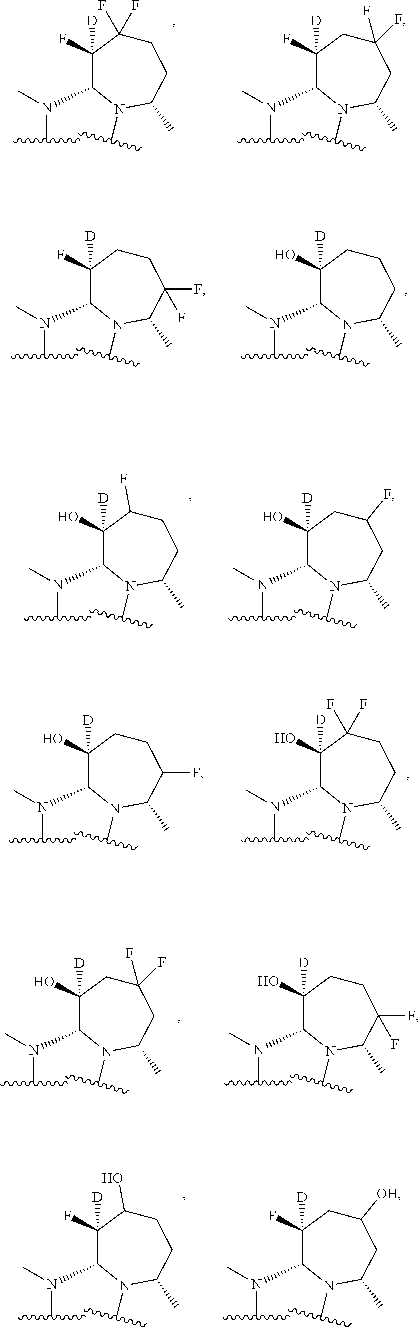

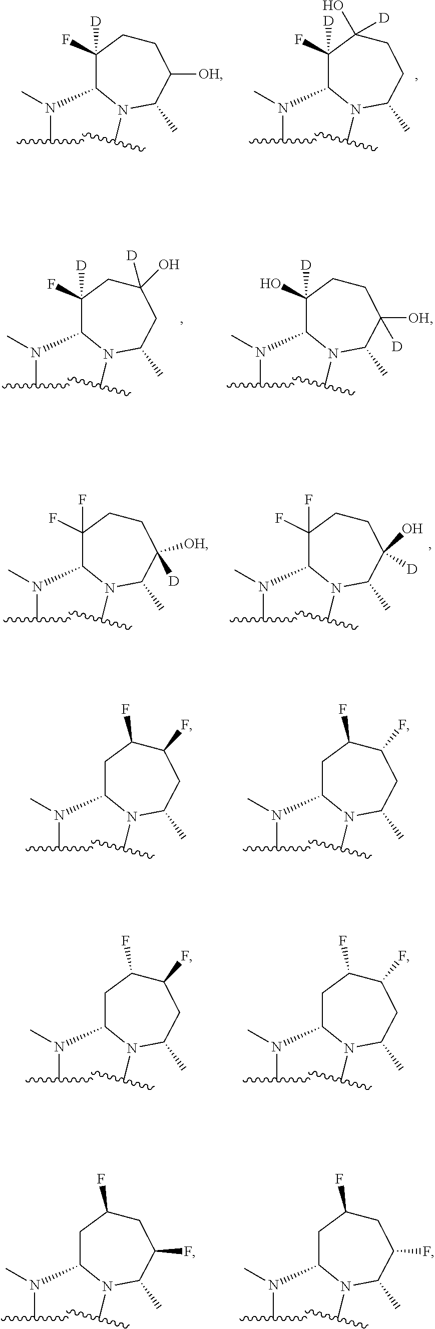

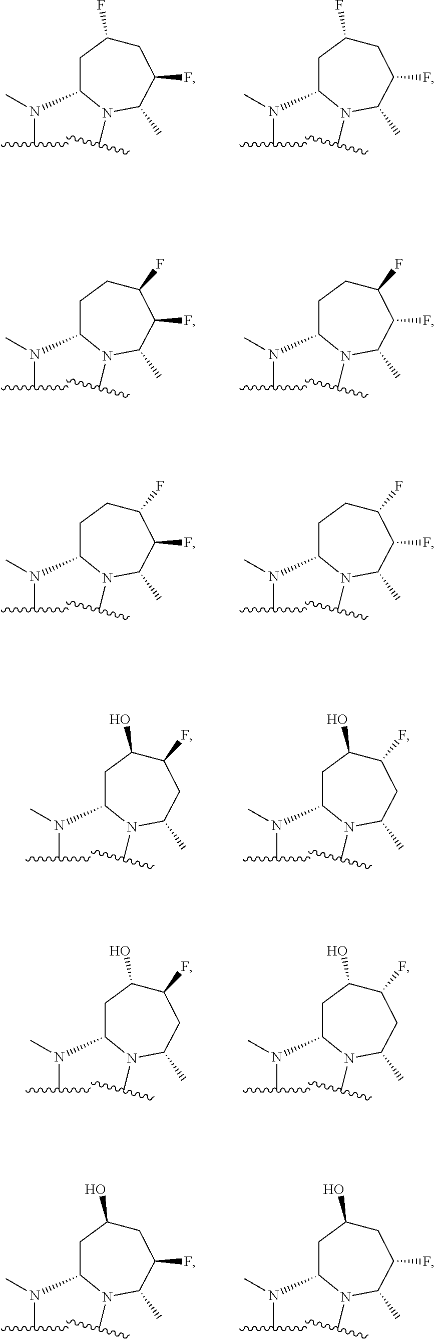

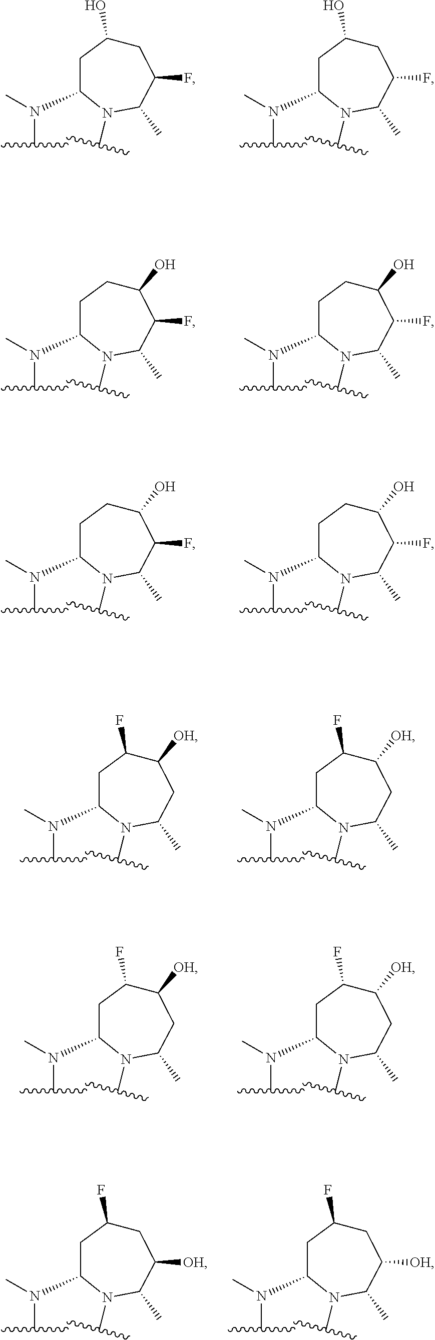

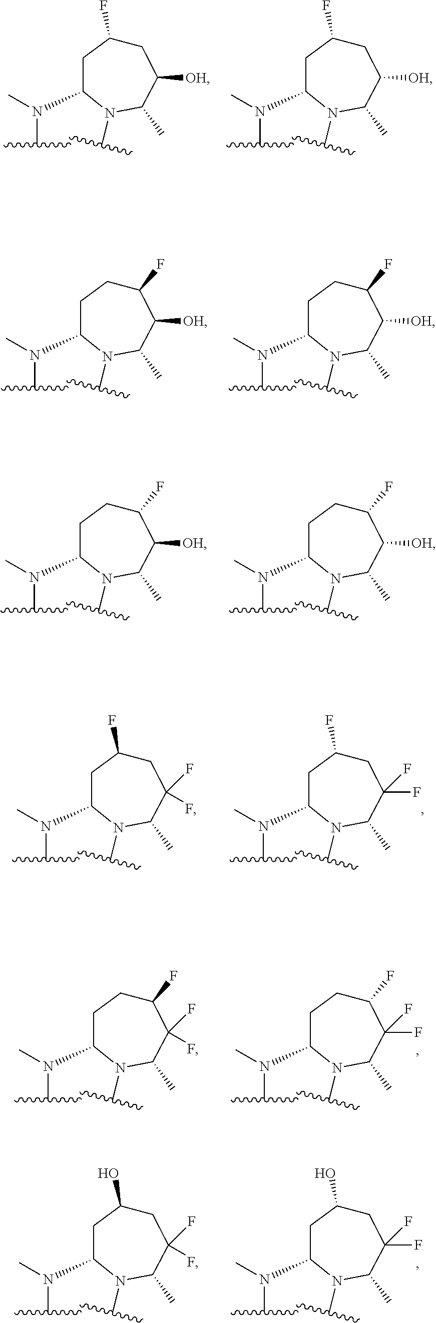

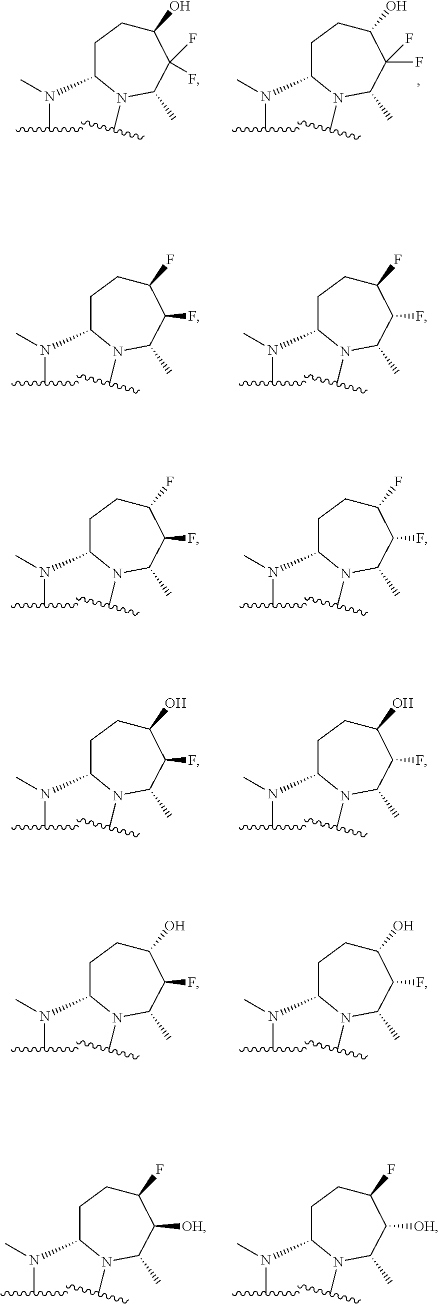

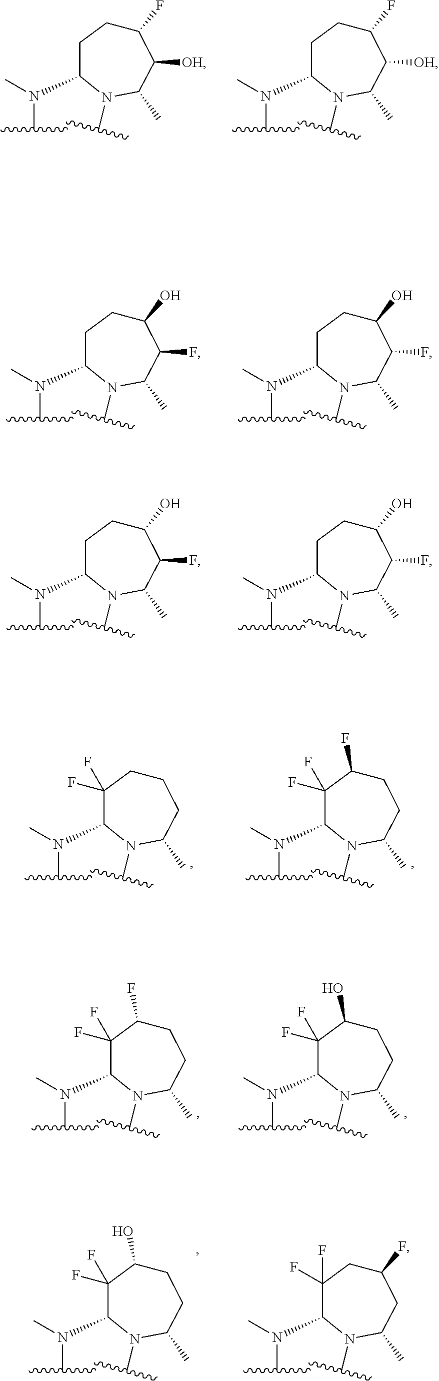

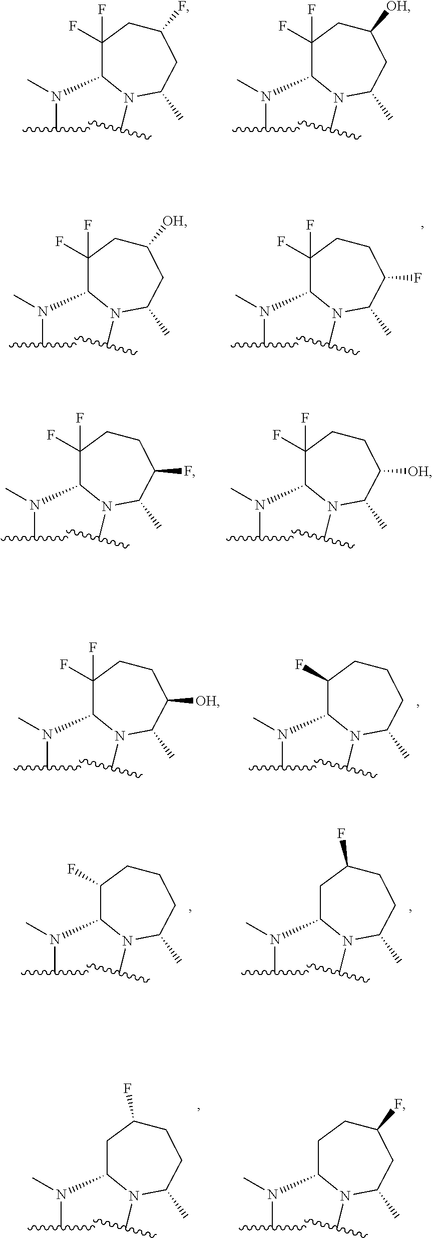

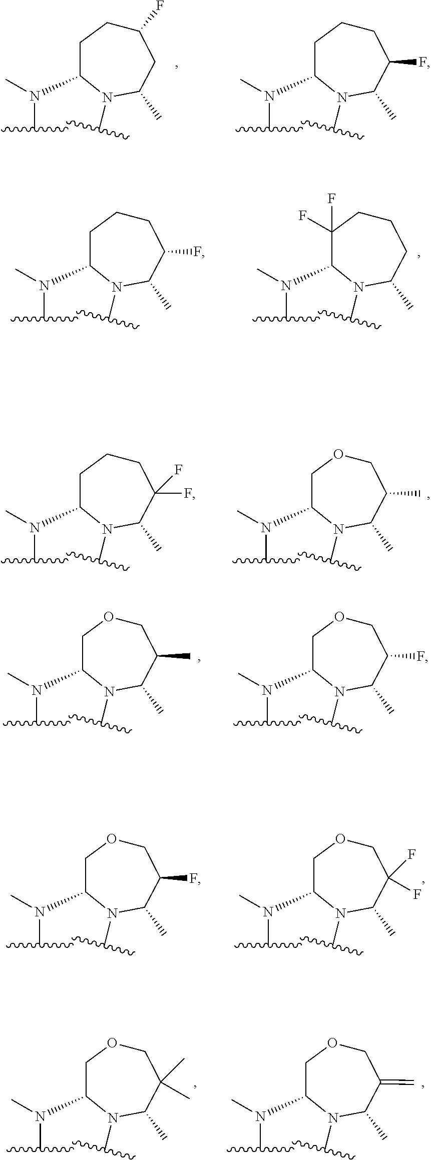

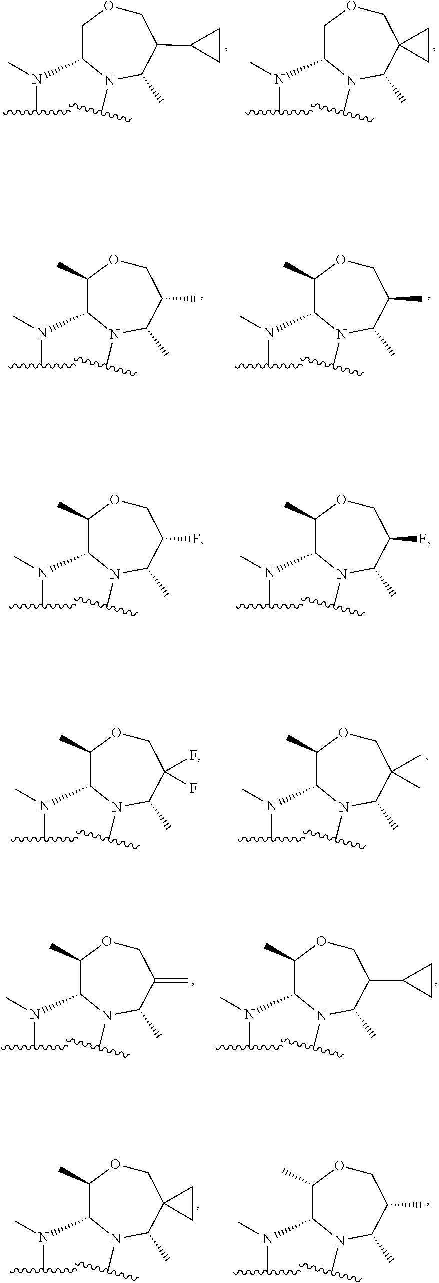

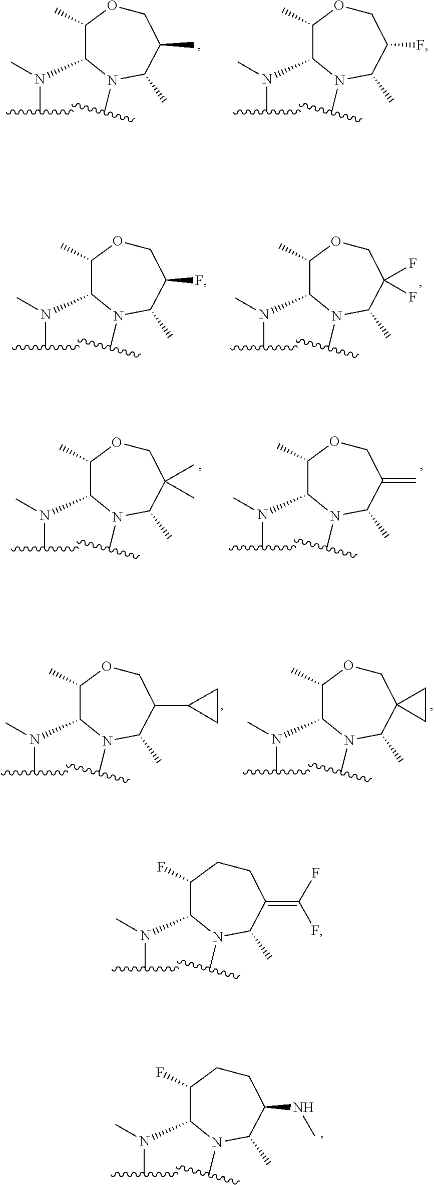

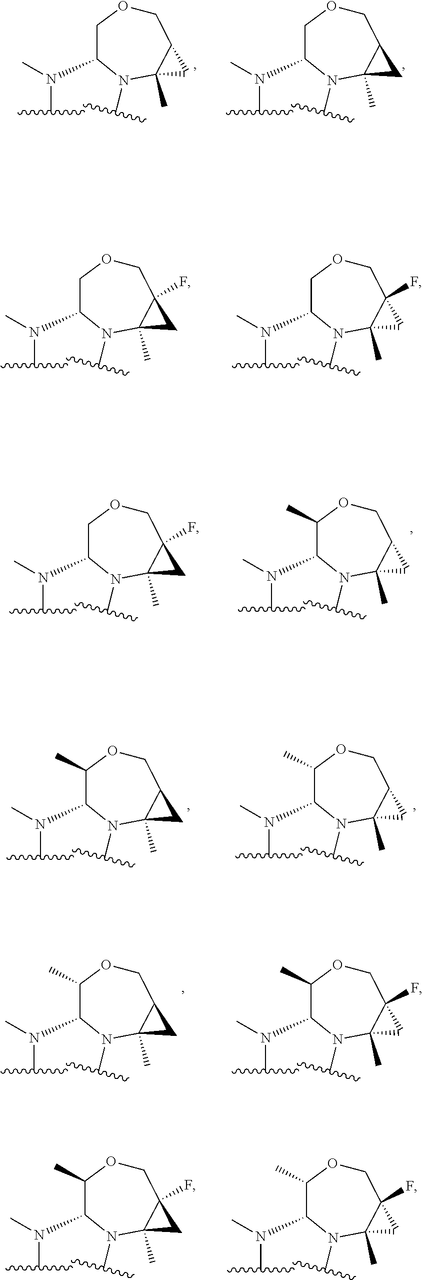

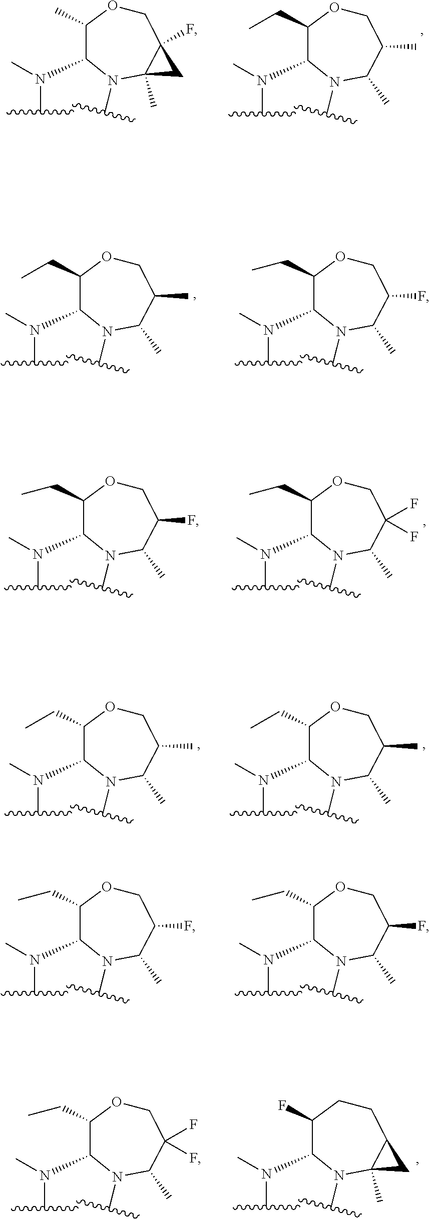

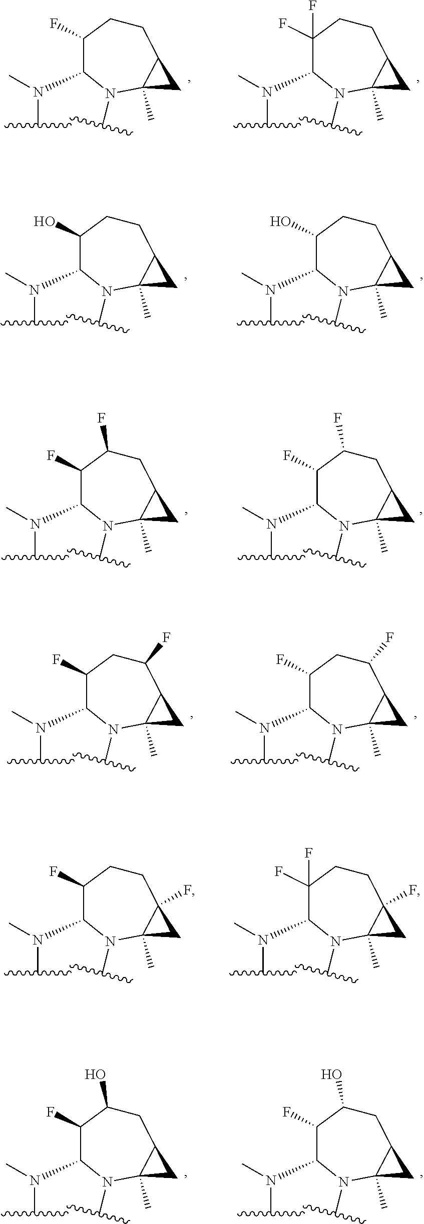

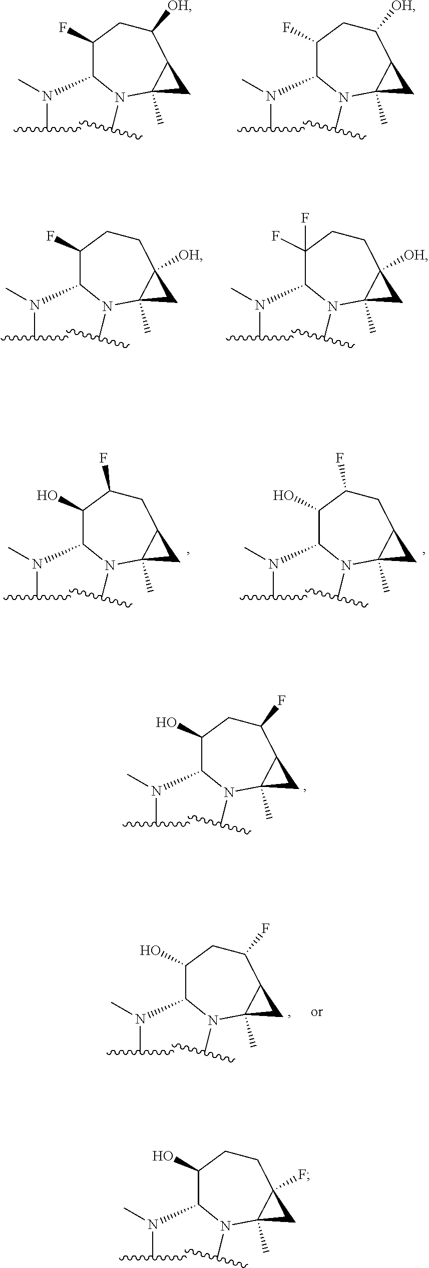

and
R₄ is:

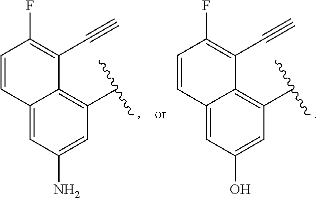 [SMILES: *c1cc(N)cc2ccc(F)c(C#C)c12 ; *c1cc(O)cc2ccc(F)c(C#C)c12]

In some embodiments, the compound is:

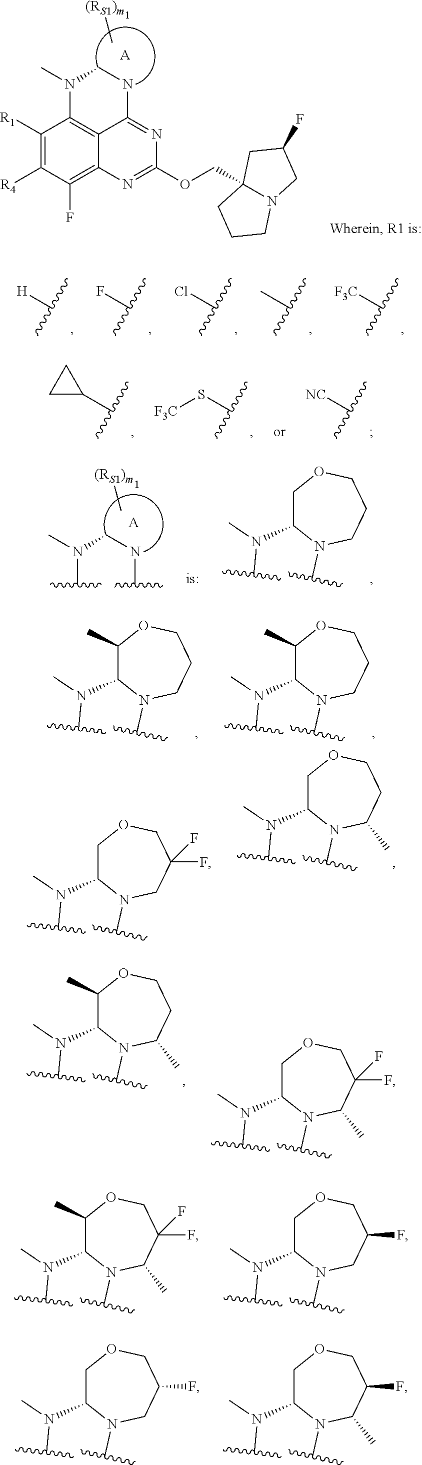

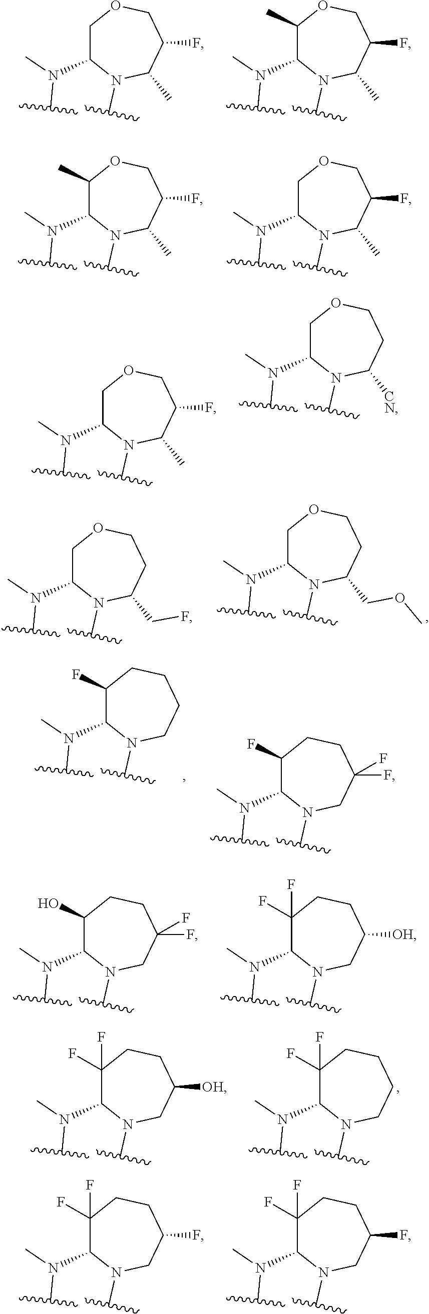

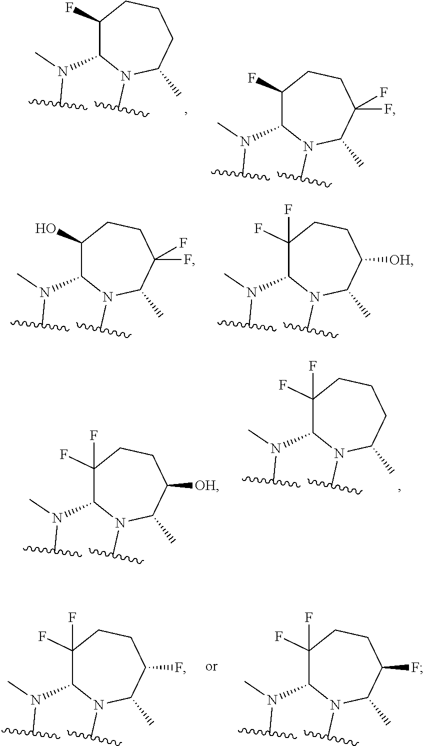

and
R₄ is:

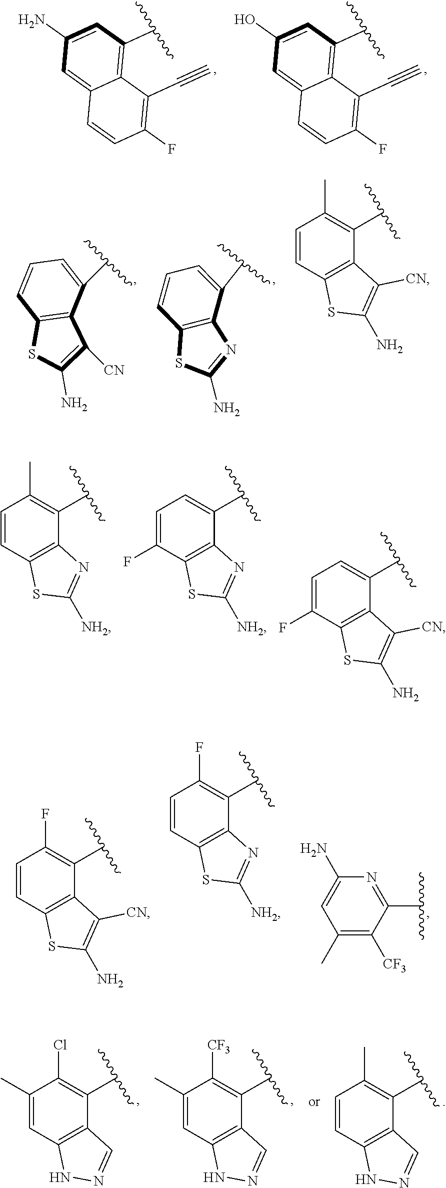

In some embodiments, the compound is:

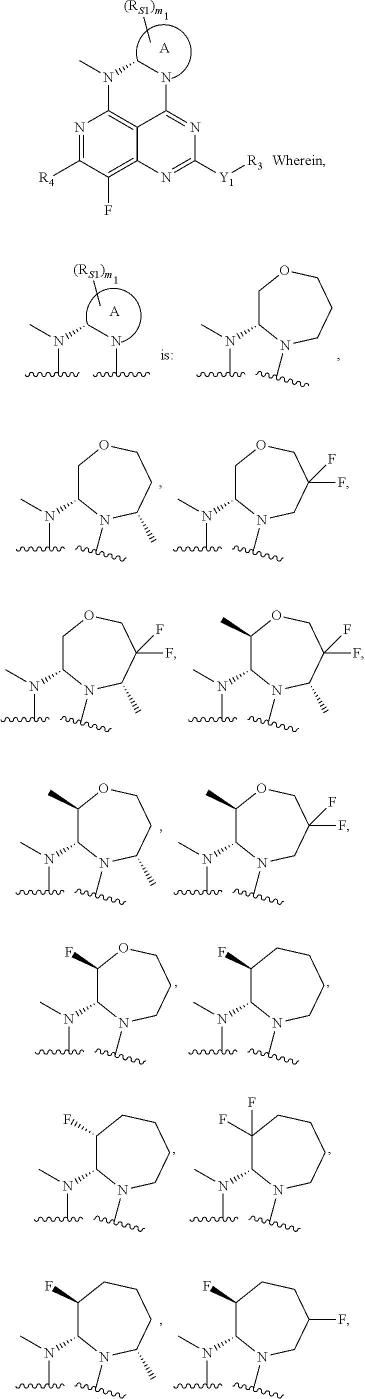

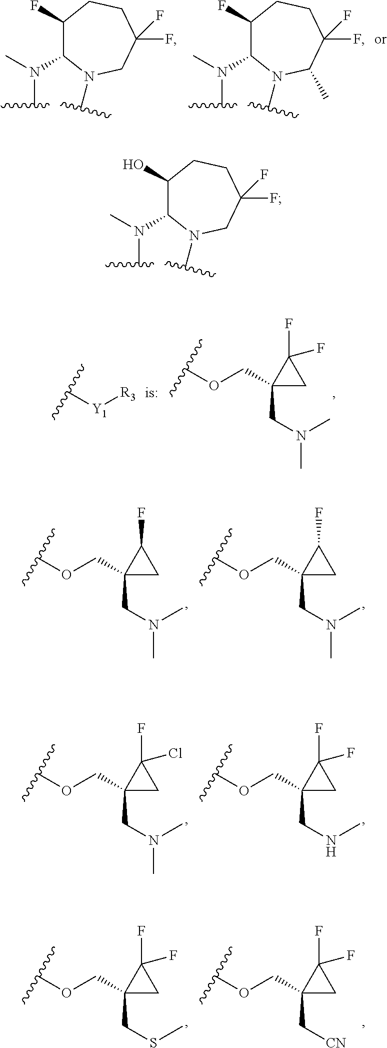

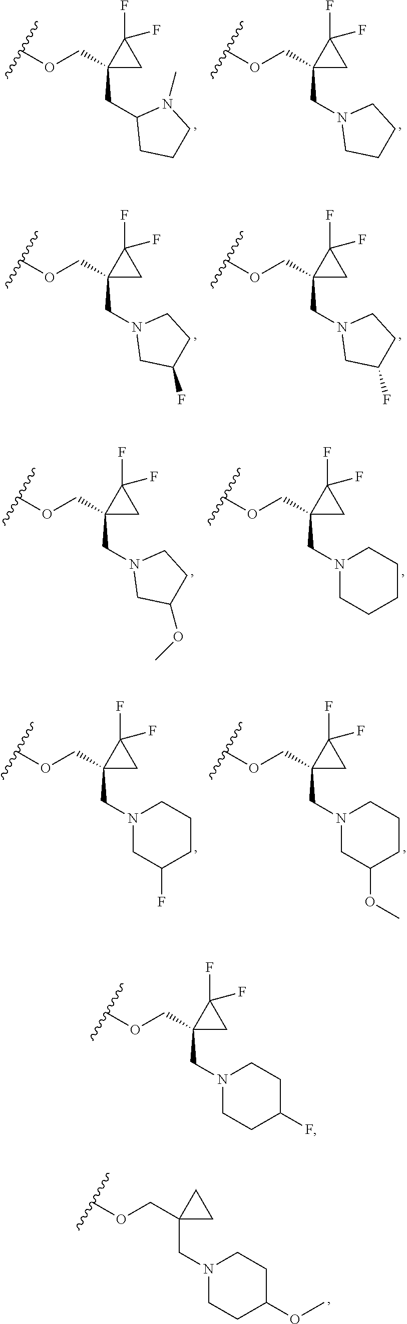

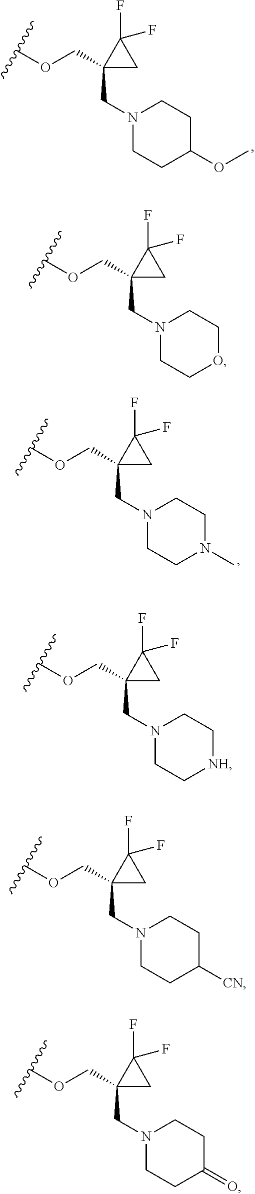 [SMILES: *OC[C@@]1(CN2CCC(OC)CC2)CC1(F)F ; *OC[C@@]1(CN2CCOCC2)CC1(F)F ; *OC[C@@]1(CN2CCN(C)CC2)CC1(F)F ; *OC[C@@]1(CN2CCNCC2)CC1(F)F ; *OC[C@@]1(CN2CCC(C#N)CC2)CC1(F)F ; *OC[C@@]1(CN2CCC(=O)CC2)CC1(F)F]

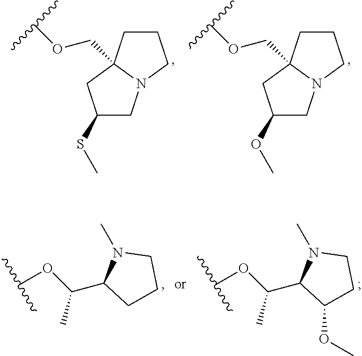

and
R₄ is:

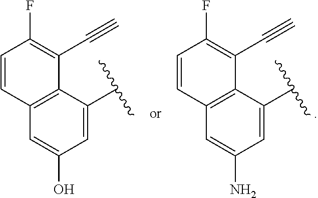 [SMILES: *c1cc(O)cc2ccc(F)c(C#C)c12 ; *c1cc(N)cc2ccc(F)c(C#C)c12]

In some embodiments, the compound is:

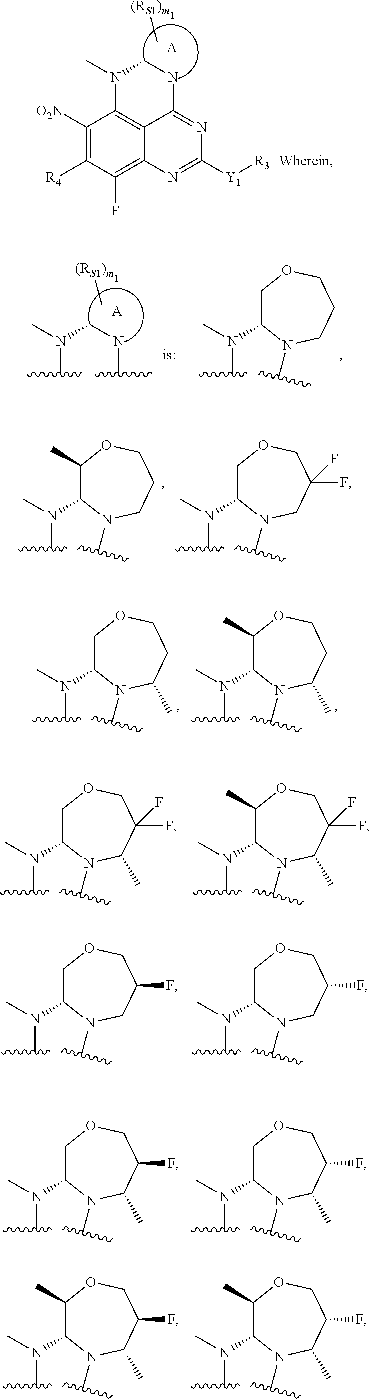

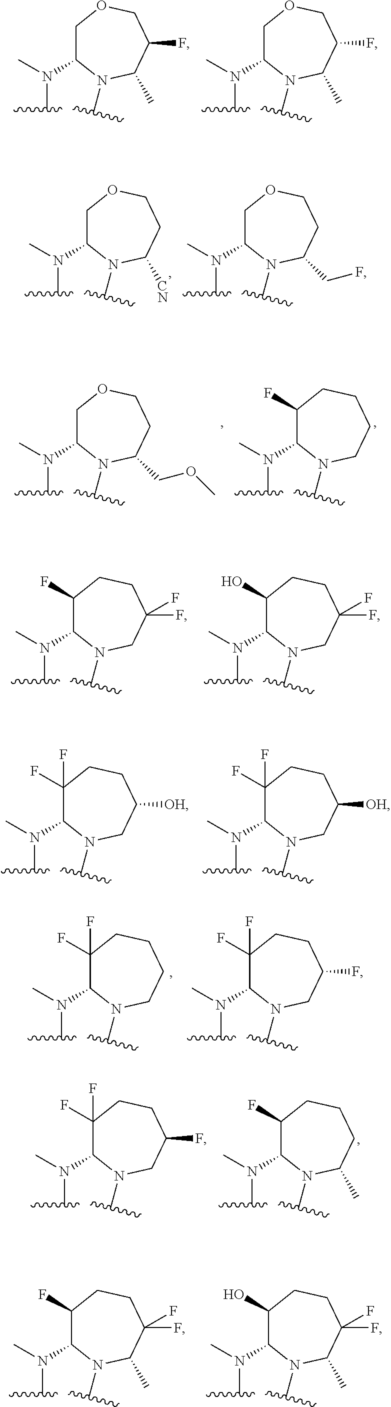

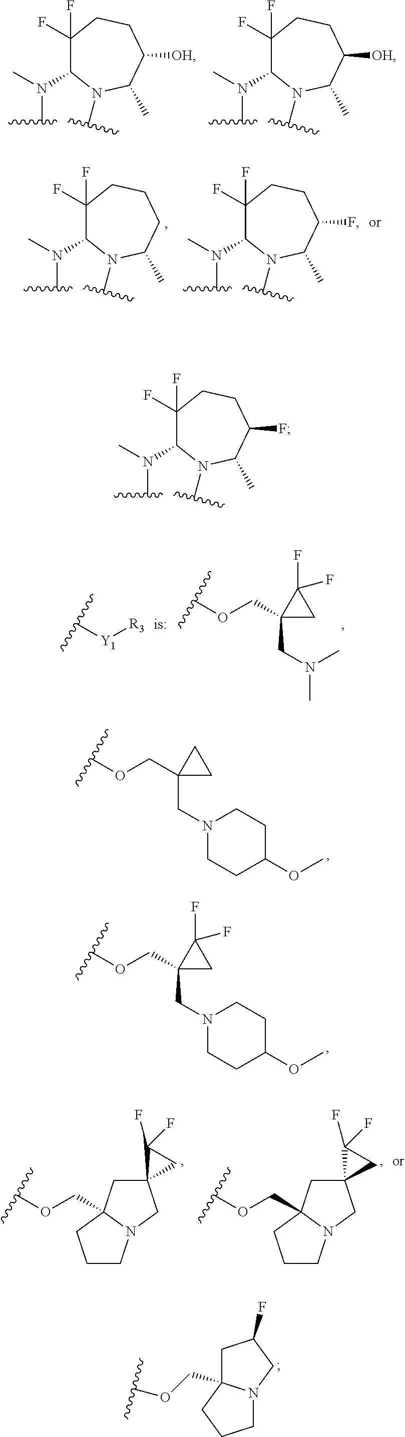

and
R₄ is:

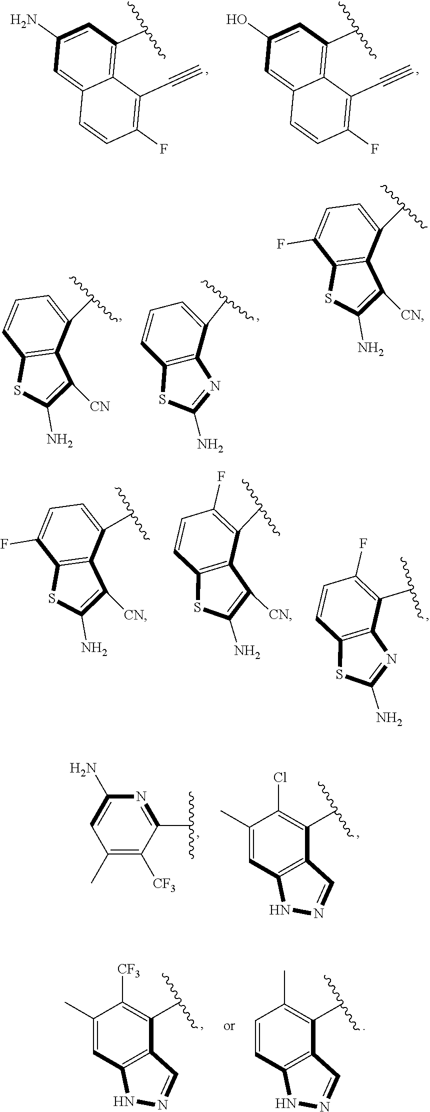

In some embodiments, the compound is:

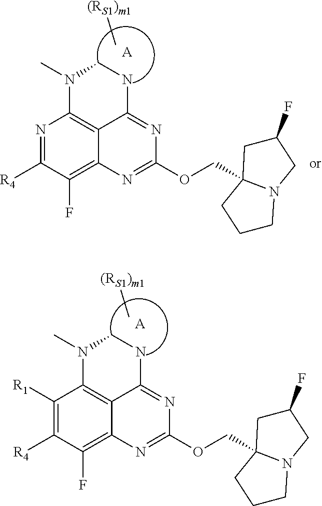

Wherein,
R₁ is:

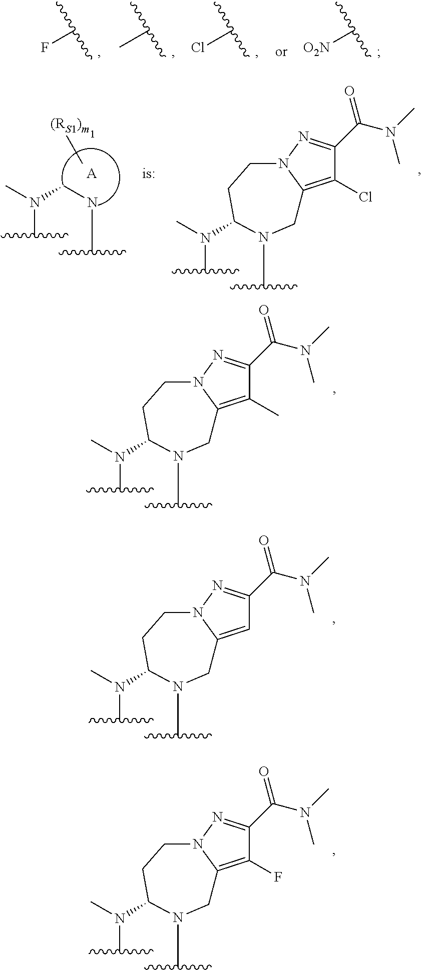

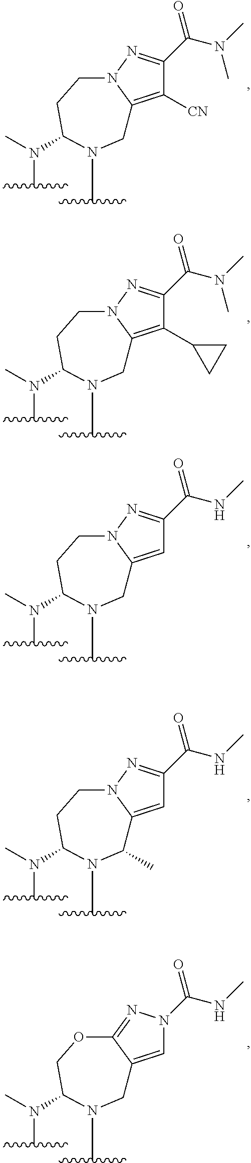

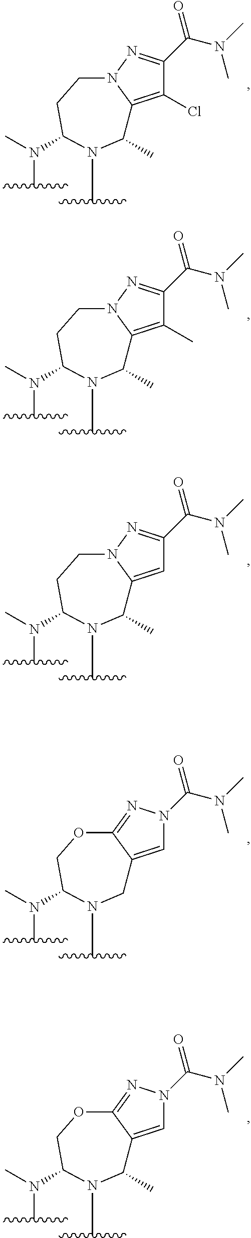

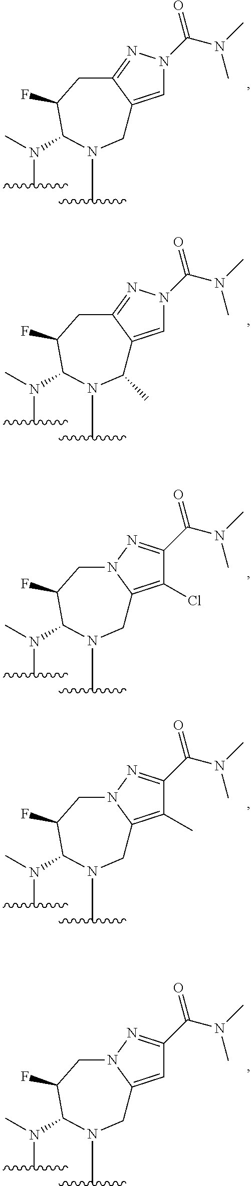

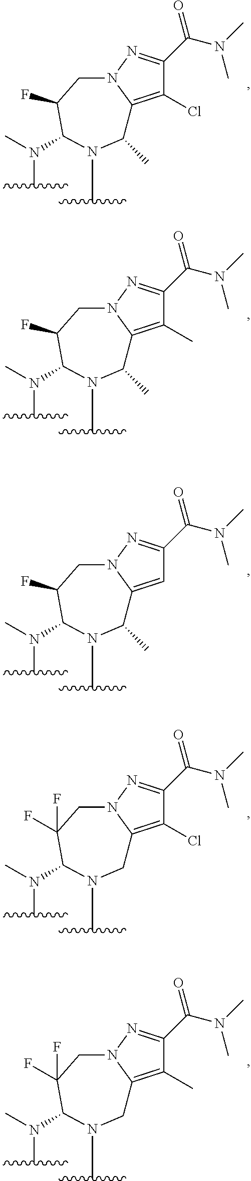

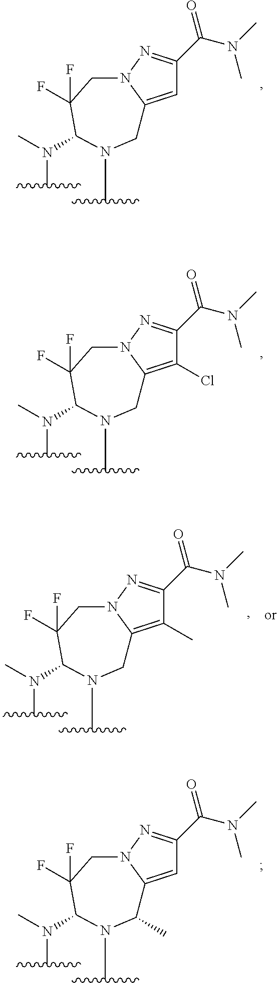

and
R₄ is:

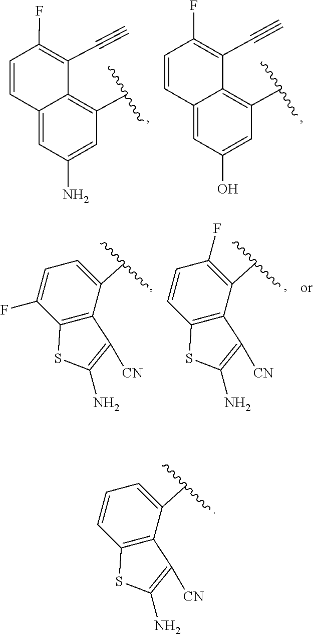

In some embodiments, the compound is:

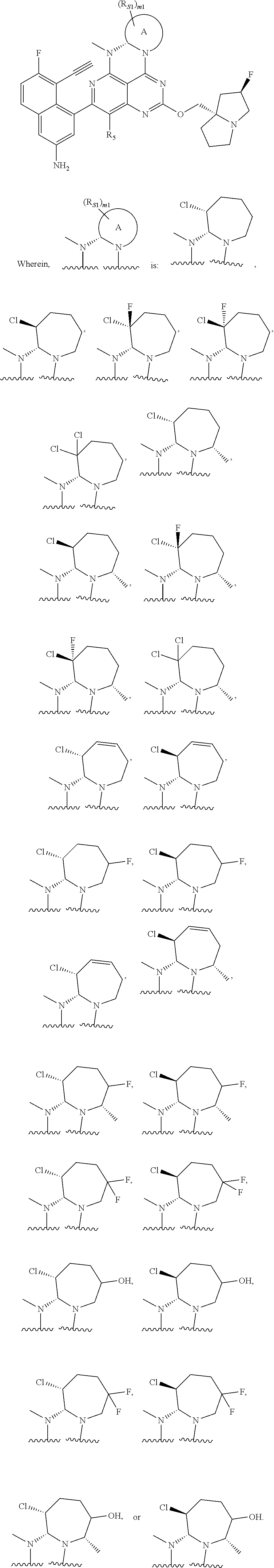

[66]. The compound of any one of [1] to [65], wherein, the compound is any one of compounds in the Table C:

TABLE C

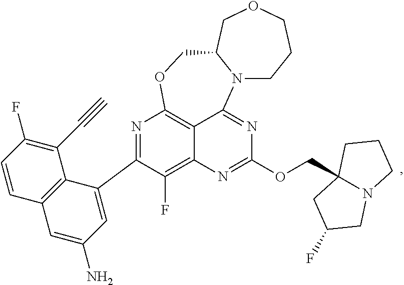 [SMILES: C#Cc1c(F)ccc2cc(N)cc(-c3nc4c5c(nc(OC[C@@]67CCCN6C[C@H](F)C7)nc5c3F)N3CCCOC[C@H]3CO4)c12]

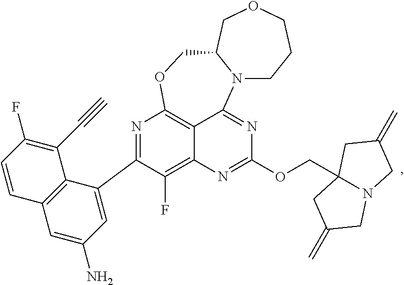 [SMILES: C#Cc1c(F)ccc2cc(N)cc(-c3nc4c5c(nc(OCC67CC(=C)CN6CC(=C)C7)nc5c3F)N3CCCOC[C@H]3CO4)c12]

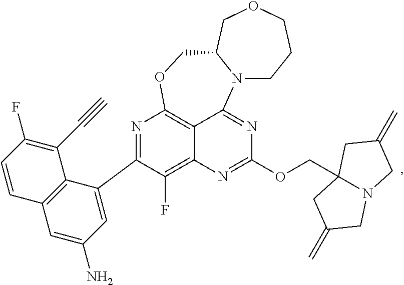 [SMILES: C#Cc1c(F)ccc2cc(N)cc(-c3nc4c5c(nc(OCC67CC(=C)CN6CC(=C)C7)nc5c3F)N3CCCOC[C@H]3CO4)c12]

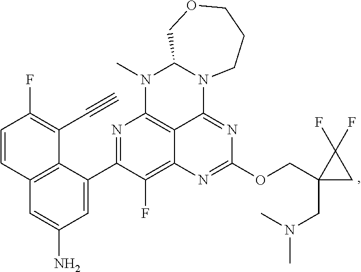 [SMILES: C#Cc1c(F)ccc2cc(N)cc(-c3nc4c5c(nc(OCC6(CN(C)C)CC6(F)F)nc5c3F)N3CCCOC[C@H]3N4C)c12]

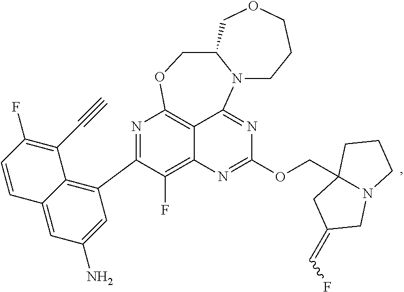 [SMILES: C#Cc1c(F)ccc2cc(N)cc(-c3nc4c5c(nc(OCC67CCCN6CC(=CF)C7)nc5c3F)N3CCCOC[C@H]3CO4)c12]

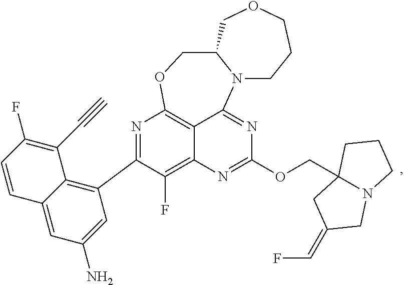 [SMILES: C#Cc1c(F)ccc2cc(N)cc(-c3nc4c5c(nc(OCC67CCCN6C/C(=C/F)C7)nc5c3F)N3CCCOC[C@H]3CO4)c12]

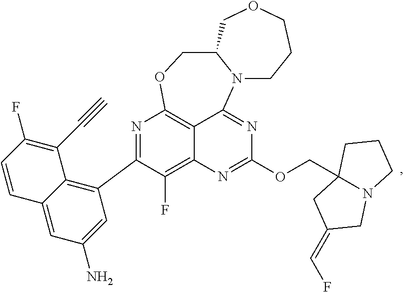 [SMILES: C#Cc1c(F)ccc2cc(N)cc(-c3nc4c5c(nc(OCC67CCCN6C/C(=C\F)C7)nc5c3F)N3CCCOC[C@H]3CO4)c12]

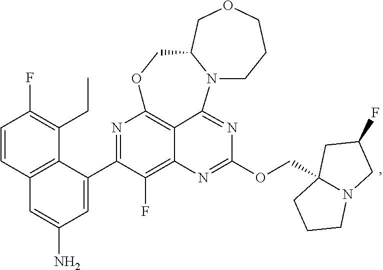 [SMILES: CCc1c(F)ccc2cc(N)cc(-c3nc4c5c(nc(OC[C@@]67CCCN6C[C@H](F)C7)nc5c3F)N3CCCOC[C@H]3CO4)c12]

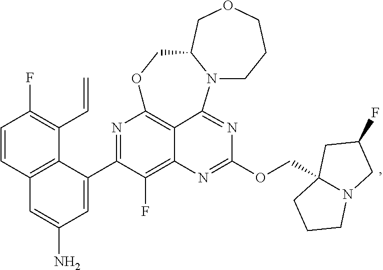 [SMILES: C=Cc1c(F)ccc2cc(N)cc(-c3nc4c5c(nc(OC[C@@]67CCCN6C[C@H](F)C7)nc5c3F)N3CCCOC[C@H]3CO4)c12]

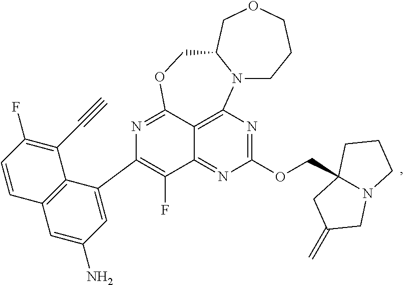 [SMILES: C#Cc1c(F)ccc2cc(N)cc(-c3nc4c5c(nc(OC[C@@]67CCCN6CC(=C)C7)nc5c3F)N3CCCOC[C@H]3CO4)c12]

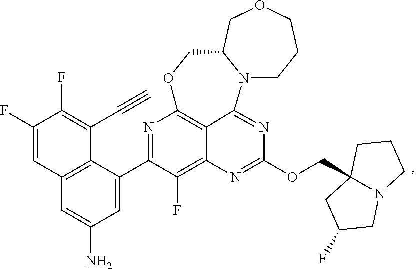 [SMILES: C#Cc1c(F)c(F)cc2cc(N)cc(-c3nc4c5c(nc(OC[C@@]67CCCN6C[C@H](F)C7)nc5c3F)N3CCCOC[C@H]3CO4)c12]

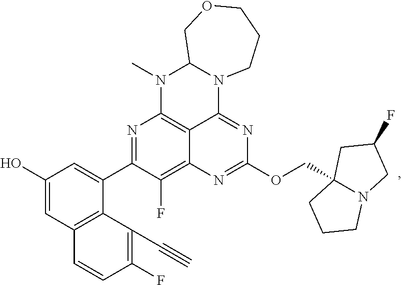 [SMILES: C#Cc1c(F)ccc2cc(O)cc(-c3nc4c5c(nc(OC[C@@]67CCCN6C[C@H](F)C7)nc5c3F)N3CCCOCC3N4C)c12]

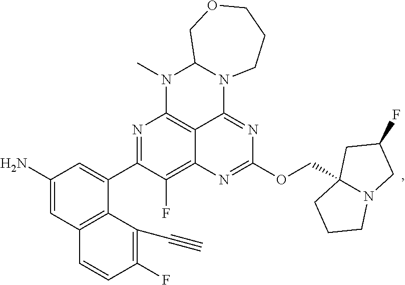 [SMILES: C#Cc1c(F)ccc2cc(N)cc(-c3nc4c5c(nc(OC[C@@]67CCCN6C[C@H](F)C7)nc5c3F)N3CCCOCC3N4C)c12]

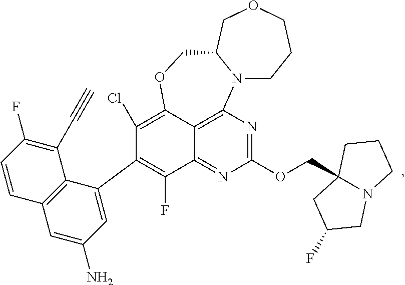 [SMILES: C#Cc1c(F)ccc2cc(N)cc(-c3c(Cl)c4c5c(nc(OC[C@@]67CCCN6C[C@H](F)C7)nc5c3F)N3CCCOC[C@H]3CO4)c12]

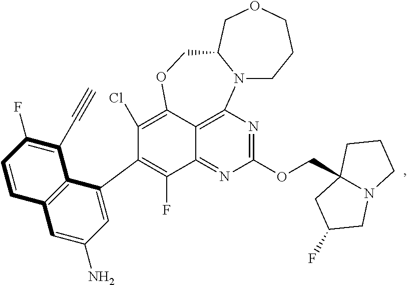

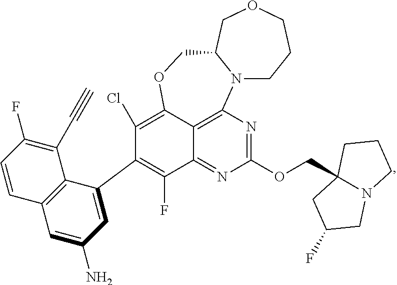

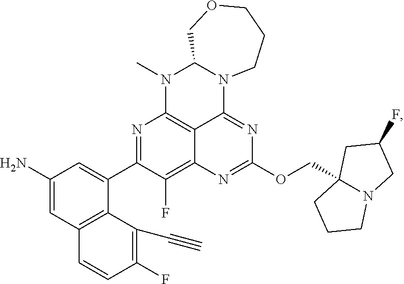 [SMILES: C#Cc1c(F)ccc2cc(N)cc(-c3nc4c5c(nc(OC[C@@]67CCCN6C[C@H](F)C7)nc5c3F)N3CCCOC[C@H]3N4C)c12]

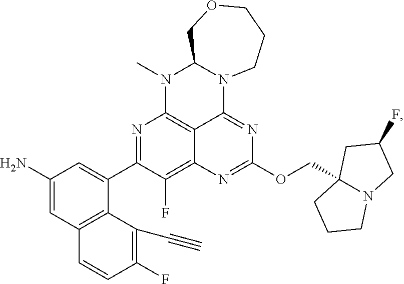 [SMILES: C#Cc1c(F)ccc2cc(N)cc(-c3nc4c5c(nc(OC[C@@]67CCCN6C[C@H](F)C7)nc5c3F)N3CCCOC[C@@H]3N4C)c12]

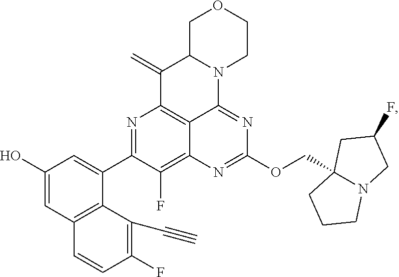 [SMILES: C#Cc1c(F)ccc2cc(O)cc(-c3nc4c5c(nc(OC[C@@]67CCCN6C[C@H](F)C7)nc5c3F)N3CCOCC3C4=C)c12]

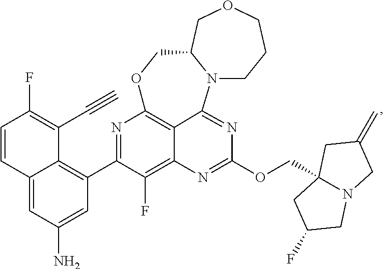 [SMILES: C#Cc1c(F)ccc2cc(N)cc(-c3nc4c5c(nc(OC[C@]67CC(=C)CN6C[C@H](F)C7)nc5c3F)N3CCCOC[C@H]3CO4)c12 ; ","]

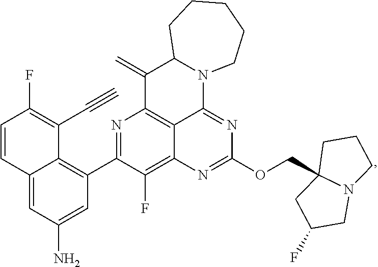 [SMILES: C#Cc1c(F)ccc2cc(N)cc(-c3nc4c5c(nc(OC[C@@]67CCCN6C[C@H](F)C7)nc5c3F)N3CCCCCC3C4=C)c12 ; ","]

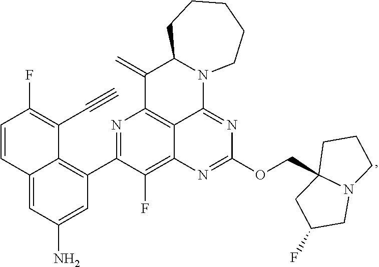 [SMILES: C#Cc1c(F)ccc2cc(N)cc(-c3nc4c5c(nc(OC[C@@]67CCCN6C[C@H](F)C7)nc5c3F)N3CCCCC[C@@H]3C4=C)c12 ; ","]

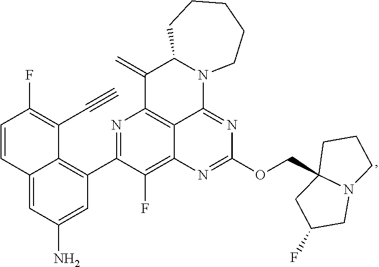 [SMILES: C#Cc1c(F)ccc2cc(N)cc(-c3nc4c5c(nc(OC[C@@]67CCCN6C[C@H](F)C7)nc5c3F)N3CCCCC[C@H]3C4=C)c12 ; ","]

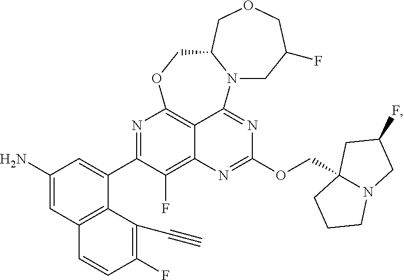 [SMILES: C#Cc1c(F)ccc2cc(N)cc(-c3nc4c5c(nc(OC[C@@]67CCCN6C[C@H](F)C7)nc5c3F)N3CC(F)COC[C@H]3CO4)c12]

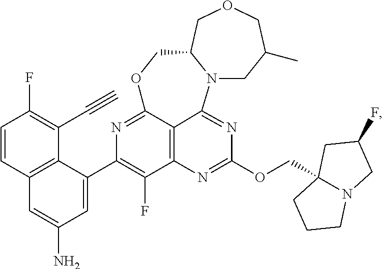 [SMILES: C#Cc1c(F)ccc2cc(N)cc(-c3nc4c5c(nc(OC[C@@]67CCCN6C[C@H](F)C7)nc5c3F)N3CC(C)COC[C@H]3CO4)c12]

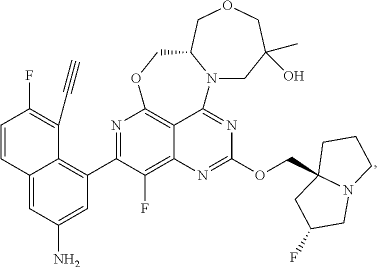 [SMILES: C#Cc1c(F)ccc2cc(N)cc(-c3nc4c5c(nc(OC[C@@]67CCCN6C[C@H](F)C7)nc5c3F)N3CC(C)(O)COC[C@H]3CO4)c12 ; ","]

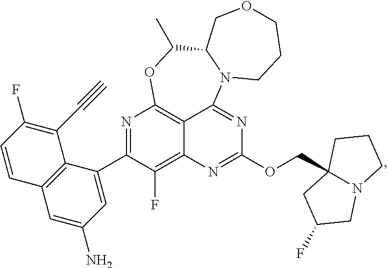

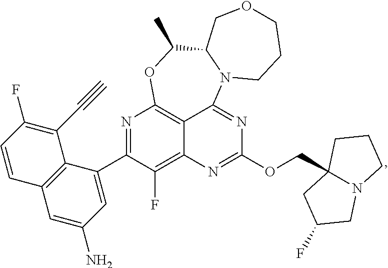 [SMILES: C#Cc1c(F)ccc2cc(N)cc(-c3nc4c5c(nc(OC[C@@]67CCCN6C[C@H](F)C7)nc5c3F)N3CCCOC[C@H]3[C@H](C)O4)c12 ; ","]

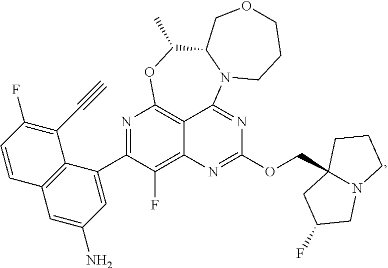 [SMILES: C#Cc1c(F)ccc2cc(N)cc(-c3nc4c5c(nc(OC[C@@]67CCCN6C[C@H](F)C7)nc5c3F)N3CCCOC[C@H]3[C@@H](C)O4)c12 ; ","]

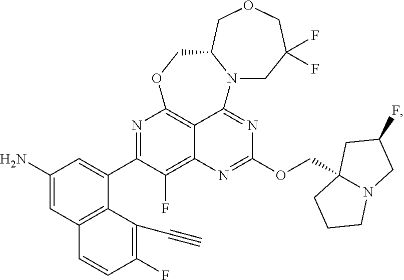 [SMILES: C#Cc1c(F)ccc2cc(N)cc(-c3nc4c5c(nc(OC[C@@]67CCCN6C[C@H](F)C7)nc5c3F)N3CC(F)(F)COC[C@H]3CO4)c12]

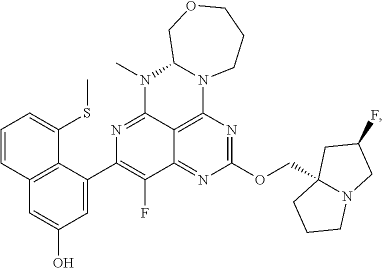 [SMILES: CSc1cccc2cc(O)cc(-c3nc4c5c(nc(OC[C@@]67CCCN6C[C@H](F)C7)nc5c3F)N3CCCOC[C@H]3N4C)c12]

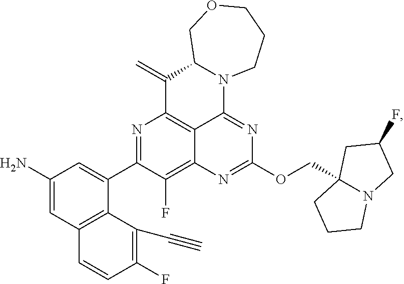 [SMILES: C#Cc1c(F)ccc2cc(N)cc(-c3nc4c5c(nc(OC[C@@]67CCCN6C[C@H](F)C7)nc5c3F)N3CCCOC[C@H]3C4=C)c12]

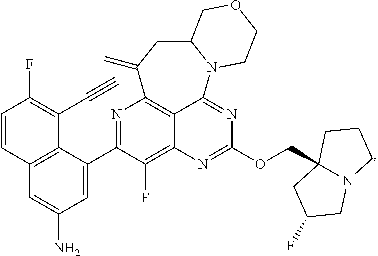 [SMILES: C#Cc1c(F)ccc2cc(N)cc(-c3nc4c5c(nc(OC[C@@]67CCCN6C[C@H](F)C7)nc5c3F)N3CCOCC3CC4=C)c12 ; ","]

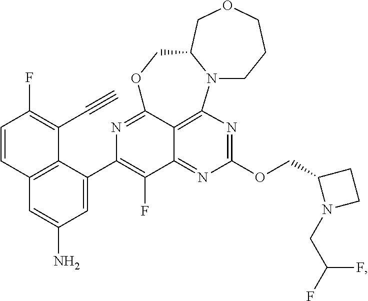 [SMILES: C#Cc1c(F)ccc2cc(N)cc(-c3nc4c5c(nc(OC[C@@H]6CCN6CC(F)F)nc5c3F)N3CCCOC[C@H]3CO4)c12]

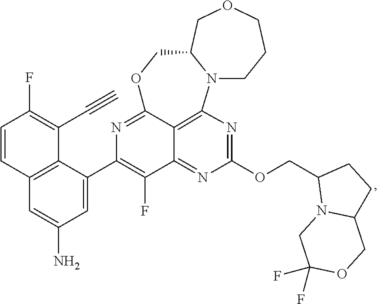 [SMILES: C#Cc1c(F)ccc2cc(N)cc(-c3nc4c5c(nc(OCC6CCC7COC(F)(F)CN67)nc5c3F)N3CCCOC[C@H]3CO4)c12 ; ","]

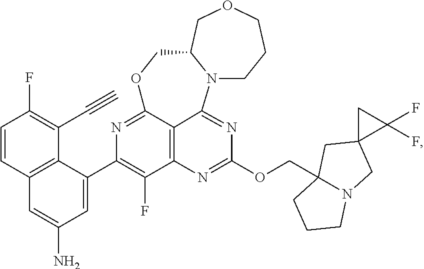 [SMILES: C#Cc1c(F)ccc2cc(N)cc(-c3nc4c5c(nc(OCC67CCCN6CC6(C7)CC6(F)F)nc5c3F)N3CCCOC[C@H]3CO4)c12]

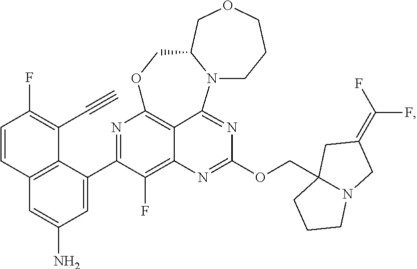 [SMILES: C#Cc1c(F)ccc2cc(N)cc(-c3nc4c5c(nc(OCC67CCCN6CC(=C(F)F)C7)nc5c3F)N3CCCOC[C@H]3CO4)c12]

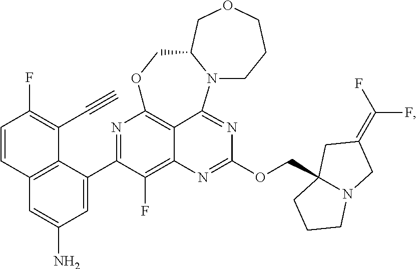 [SMILES: C#Cc1c(F)ccc2cc(N)cc(-c3nc4c5c(nc(OC[C@]67CCCN6CC(=C(F)F)C7)nc5c3F)N3CCCOC[C@H]3CO4)c12]

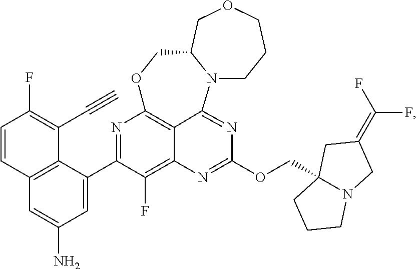 [SMILES: C#Cc1c(F)ccc2cc(N)cc(-c3nc4c5c(nc(OC[C@@]67CCCN6CC(=C(F)F)C7)nc5c3F)N3CCCOC[C@H]3CO4)c12]

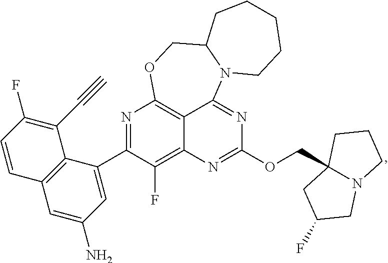 [SMILES: C#Cc1c(F)ccc2cc(N)cc(-c3nc4c5c(nc(OC[C@@]67CCCN6C[C@H](F)C7)nc5c3F)N3CCCCCC3CO4)c12 ; ","]

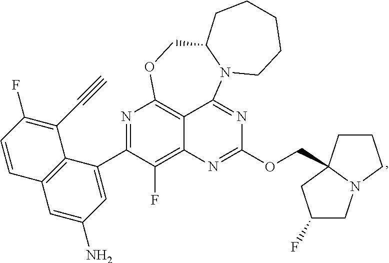 [SMILES: C#Cc1c(F)ccc2cc(N)cc(-c3nc4c5c(nc(OC[C@@]67CCCN6C[C@H](F)C7)nc5c3F)N3CCCCC[C@H]3CO4)c12 ; ","]

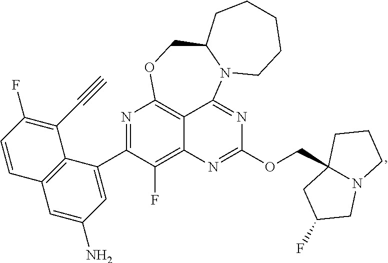 [SMILES: C#Cc1c(F)ccc2cc(N)cc(-c3nc4c5c(nc(OC[C@@]67CCCN6C[C@H](F)C7)nc5c3F)N3CCCCC[C@@H]3CO4)c12 ; ","]

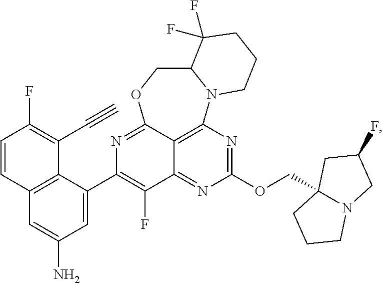 [SMILES: C#Cc1c(F)ccc2cc(N)cc(-c3nc4c5c(nc(OC[C@@]67CCCN6C[C@H](F)C7)nc5c3F)N3CCCC(F)(F)C3CO4)c12]

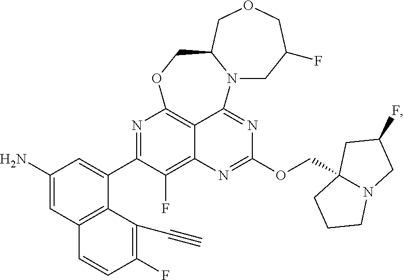 [SMILES: C#Cc1c(F)ccc2cc(N)cc(-c3nc4c5c(nc(OC[C@@]67CCCN6C[C@H](F)C7)nc5c3F)N3CC(F)COC[C@@H]3CO4)c12]

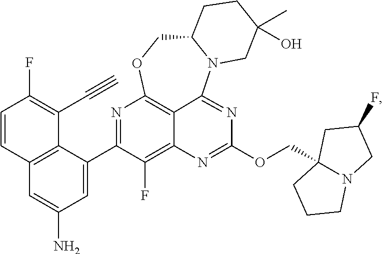 [SMILES: C#Cc1c(F)ccc2cc(N)cc(-c3nc4c5c(nc(OC[C@@]67CCCN6C[C@H](F)C7)nc5c3F)N3CC(C)(O)CC[C@H]3CO4)c12]

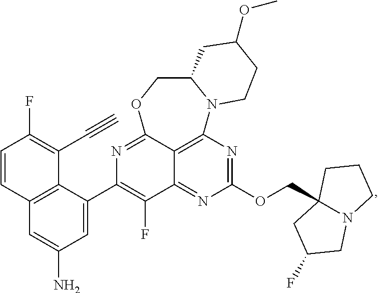

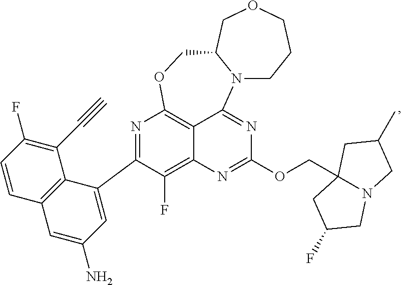 [SMILES: C#Cc1c(F)ccc2cc(N)cc(-c3nc4c5c(nc(OCC67CC(C)CN6C[C@H](F)C7)nc5c3F)N3CCCOC[C@H]3CO4)c12 ; ","]

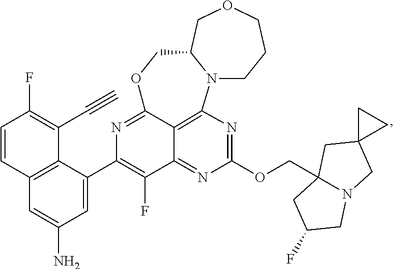 [SMILES: C#Cc1c(F)ccc2cc(N)cc(-c3nc4c5c(nc(OCC67C[C@@H](F)CN6CC6(CC6)C7)nc5c3F)N3CCCOC[C@H]3CO4)c12 ; ","]

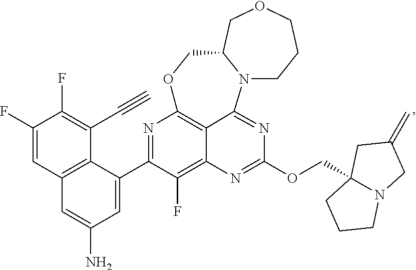 [SMILES: C#Cc1c(F)c(F)cc2cc(N)cc(-c3nc4c5c(nc(OC[C@@]67CCCN6CC(=C)C7)nc5c3F)N3CCCOC[C@H]3CO4)c12 ; ","]

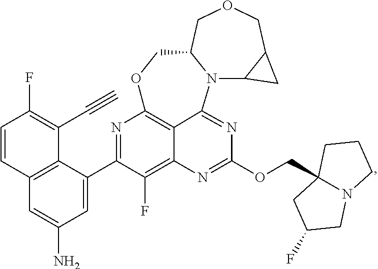 [SMILES: C#Cc1c(F)ccc2cc(N)cc(-c3nc4c5c(nc(OC[C@@]67CCCN6C[C@H](F)C7)nc5c3F)N3C5CC5COC[C@H]3CO4)c12 ; ","]

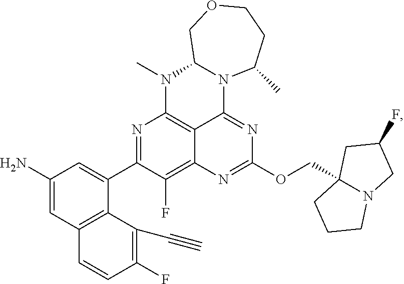 [SMILES: C#Cc1c(F)ccc2cc(N)cc(-c3nc4c5c(nc(OC[C@@]67CCCN6C[C@H](F)C7)nc5c3F)N3[C@@H](C)CCOC[C@H]3N4C)c12]

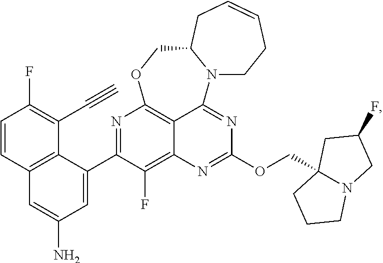 [SMILES: C#Cc1c(F)ccc2cc(N)cc(-c3nc4c5c(nc(OC[C@@]67CCCN6C[C@H](F)C7)nc5c3F)N3CCC=CC[C@H]3CO4)c12]

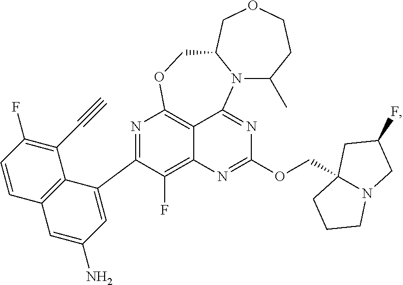 [SMILES: C#Cc1c(F)ccc2cc(N)cc(-c3nc4c5c(nc(OC[C@@]67CCCN6C[C@H](F)C7)nc5c3F)N3C(C)CCOC[C@H]3CO4)c12]

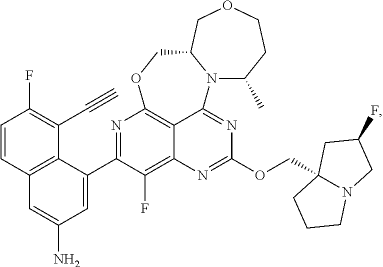 [SMILES: C#Cc1c(F)ccc2cc(N)cc(-c3nc4c5c(nc(OC[C@@]67CCCN6C[C@H](F)C7)nc5c3F)N3[C@@H](COCC[C@@H]3C)CO4)c12]

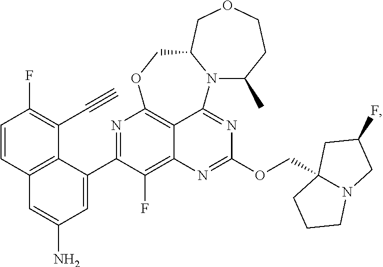 [SMILES: C#Cc1c(F)ccc2cc(N)cc(-c3nc4c5c(nc(OC[C@@]67CCCN6C[C@H](F)C7)nc5c3F)N3[C@@H](COCC[C@H]3C)CO4)c12]

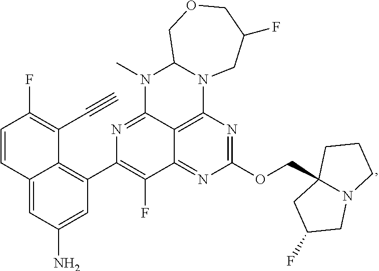 [SMILES: C#Cc1c(F)ccc2cc(N)cc(-c3nc4c5c(nc(OC[C@@]67CCCN6C[C@H](F)C7)nc5c3F)N3CC(F)COCC3N4C)c12 ; ","]

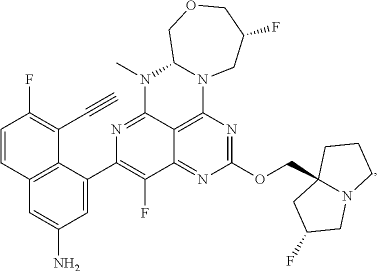 [SMILES: C#Cc1c(F)ccc2cc(N)cc(-c3nc4c5c(nc(OC[C@@]67CCCN6C[C@H](F)C7)nc5c3F)N3C[C@@H](F)COC[C@H]3N4C)c12 ; ","]

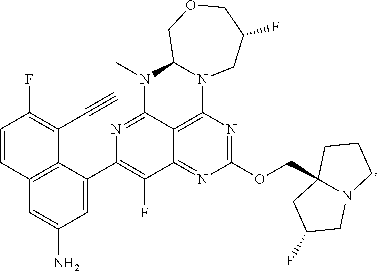 [SMILES: C#Cc1c(F)ccc2cc(N)cc(-c3nc4c5c(nc(OC[C@@]67CCCN6C[C@H](F)C7)nc5c3F)N3C[C@@H](F)COC[C@@H]3N4C)c12 ; ","]

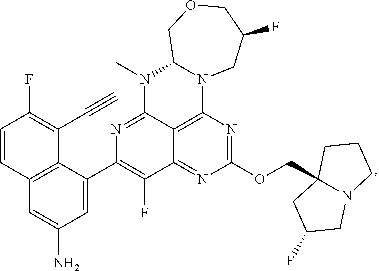 [SMILES: C#Cc1c(F)ccc2cc(N)cc(-c3nc4c5c(nc(OC[C@@]67CCCN6C[C@H](F)C7)nc5c3F)N3C[C@H](F)COC[C@H]3N4C)c12 ; ","]

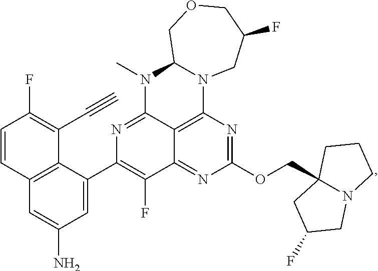 [SMILES: C#Cc1c(F)ccc2cc(N)cc(-c3nc4c5c(nc(OC[C@@]67CCCN6C[C@H](F)C7)nc5c3F)N3C[C@H](F)COC[C@@H]3N4C)c12 ; ","]

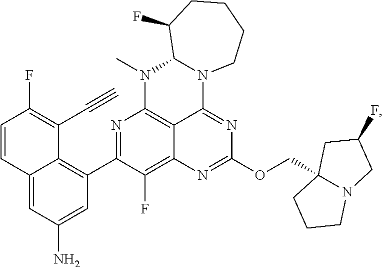 [SMILES: C#Cc1c(F)ccc2cc(N)cc(-c3nc4c5c(nc(OC[C@@]67CCCN6C[C@H](F)C7)nc5c3F)N3CCCC[C@H](F)[C@H]3N4C)c12]

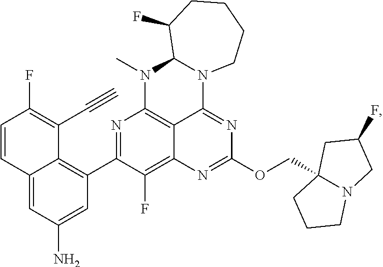 [SMILES: C#Cc1c(F)ccc2cc(N)cc(-c3nc4c5c(nc(OC[C@@]67CCCN6C[C@H](F)C7)nc5c3F)N3CCCC[C@H](F)[C@@H]3N4C)c12]

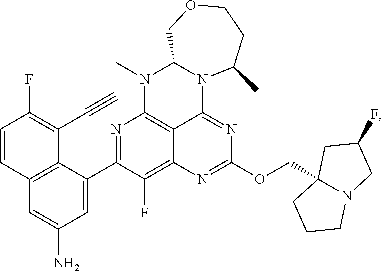 [SMILES: C#Cc1c(F)ccc2cc(N)cc(-c3nc4c5c(nc(OC[C@@]67CCCN6C[C@H](F)C7)nc5c3F)N3[C@H](C)CCOC[C@H]3N4C)c12]

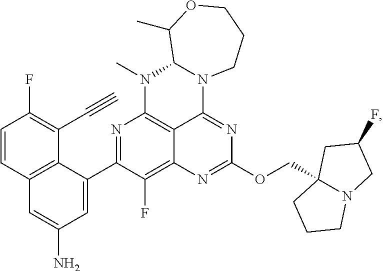 [SMILES: C#Cc1c(F)ccc2cc(N)cc(-c3nc4c5c(nc(OC[C@@]67CCCN6C[C@H](F)C7)nc5c3F)N3CCCOC(C)[C@H]3N4C)c12]

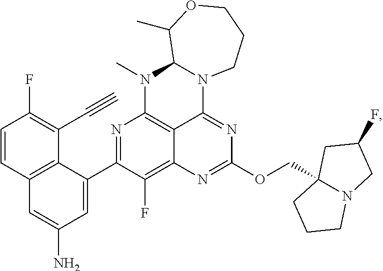 [SMILES: C#Cc1c(F)ccc2cc(N)cc(-c3nc4c5c(nc(OC[C@@]67CCCN6C[C@H](F)C7)nc5c3F)N3CCCOC(C)[C@@H]3N4C)c12]

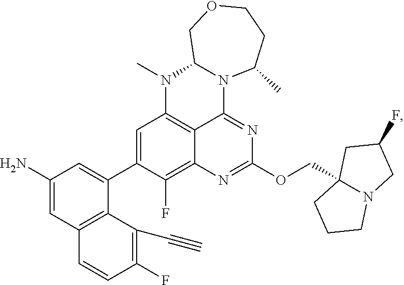 [SMILES: C#Cc1c(F)ccc2cc(N)cc(-c3cc4c5c(nc(OC[C@@]67CCCN6C[C@H](F)C7)nc5c3F)N3[C@@H](C)CCOC[C@H]3N4C)c12]

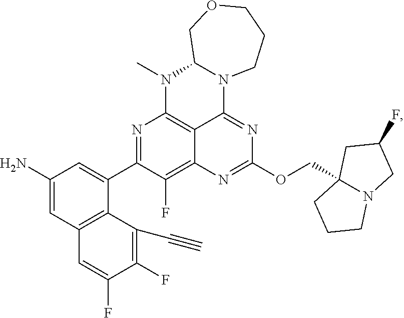 [SMILES: C#Cc1c(F)c(F)cc2cc(N)cc(-c3nc4c5c(nc(OC[C@@]67CCCN6C[C@H](F)C7)nc5c3F)N3CCCOC[C@H]3N4C)c12]

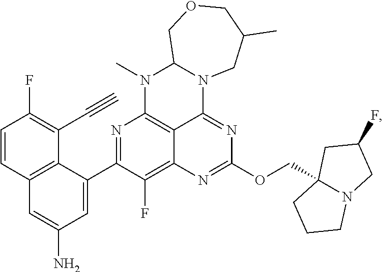 [SMILES: C#Cc1c(F)ccc2cc(N)cc(-c3nc4c5c(nc(OC[C@@]67CCCN6C[C@H](F)C7)nc5c3F)N3CC(C)COCC3N4C)c12]

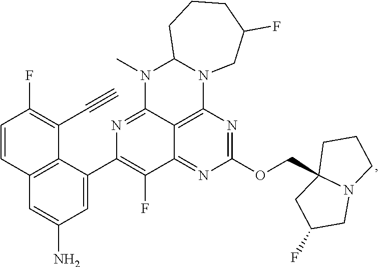 [SMILES: C#Cc1c(F)ccc2cc(N)cc(-c3nc4c5c(nc(OC[C@@]67CCCN6C[C@H](F)C7)nc5c3F)N3CC(F)CCCC3N4C)c12]

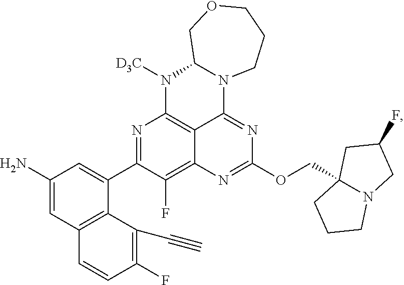 [SMILES: [2H]C([2H])([2H])N1c2nc(-c3cc(N)cc4ccc(F)c(C#C)c34)c(F)c3nc(OC[C@@]45CCCN4C[C@H](F)C5)nc(c23)N2CCCOC[C@H]21]

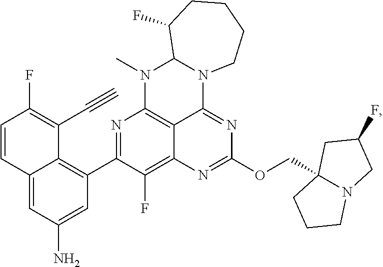 [SMILES: C#Cc1c(F)ccc2cc(N)cc(-c3nc4c5c(nc(OC[C@@]67CCCN6C[C@H](F)C7)nc5c3F)N3CCCC[C@@H](F)C3N4C)c12]

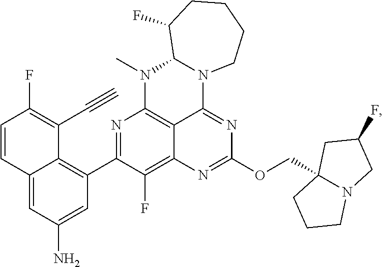 [SMILES: C#Cc1c(F)ccc2cc(N)cc(-c3nc4c5c(nc(OC[C@@]67CCCN6C[C@H](F)C7)nc5c3F)N3CCCC[C@@H](F)[C@H]3N4C)c12]

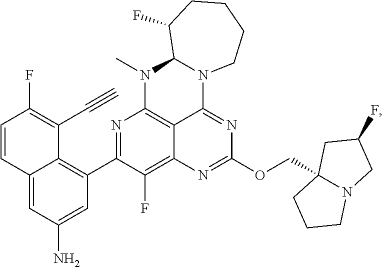 [SMILES: C#Cc1c(F)ccc2cc(N)cc(-c3nc4c5c(nc(OC[C@@]67CCCN6C[C@H](F)C7)nc5c3F)N3CCCC[C@@H](F)[C@@H]3N4C)c12]

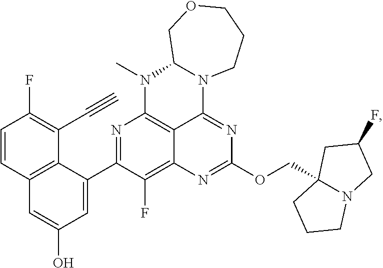 [SMILES: C#Cc1c(F)ccc2cc(O)cc(-c3nc4c5c(nc(OC[C@@]67CCCN6C[C@H](F)C7)nc5c3F)N3CCCOC[C@H]3N4C)c12]

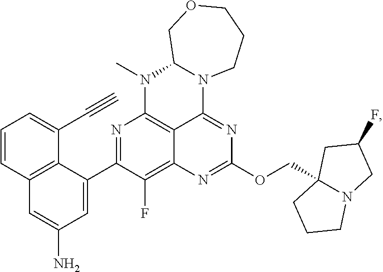 [SMILES: C#Cc1cccc2cc(N)cc(-c3nc4c5c(nc(OC[C@@]67CCCN6C[C@H](F)C7)nc5c3F)N3CCCOC[C@H]3N4C)c12]

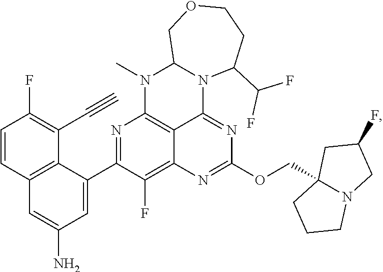 [SMILES: C#Cc1c(F)ccc2cc(N)cc(-c3nc4c5c(nc(OC[C@@]67CCCN6C[C@H](F)C7)nc5c3F)N3C(C(F)F)CCOCC3N4C)c12]

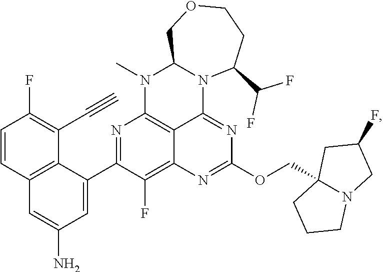 [SMILES: C#Cc1c(F)ccc2cc(N)cc(-c3nc4c5c(nc(OC[C@@]67CCCN6C[C@H](F)C7)nc5c3F)N3[C@H](COCC[C@H]3C(F)F)N4C)c12]

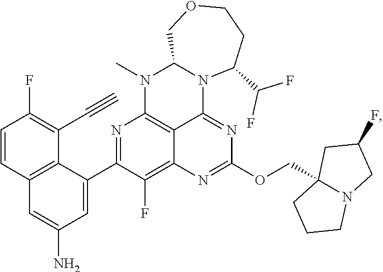 [SMILES: C#Cc1c(F)ccc2cc(N)cc(-c3nc4c5c(nc(OC[C@@]67CCCN6C[C@H](F)C7)nc5c3F)N3[C@@H](C(F)F)CCOC[C@H]3N4C)c12]

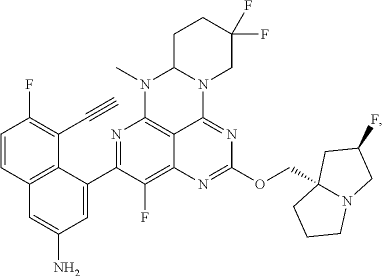 [SMILES: C#Cc1c(F)ccc2cc(N)cc(-c3nc4c5c(nc(OC[C@@]67CCCN6C[C@H](F)C7)nc5c3F)N3CC(F)(F)CCC3N4C)c12]

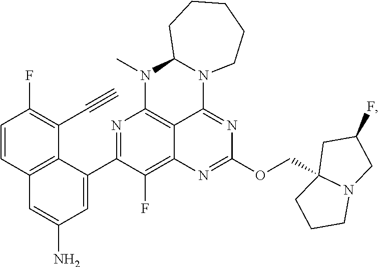 [SMILES: C#Cc1c(F)ccc2cc(N)cc(-c3nc4c5c(nc(OC[C@@]67CCCN6C[C@H](F)C7)nc5c3F)N3CCCCC[C@@H]3N4C)c12]

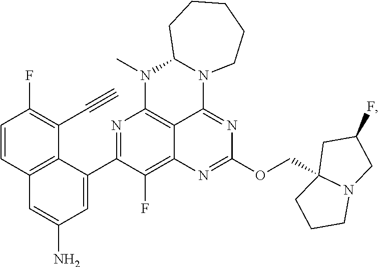 [SMILES: C#Cc1c(F)ccc2cc(N)cc(-c3nc4c5c(nc(OC[C@@]67CCCN6C[C@H](F)C7)nc5c3F)N3CCCCC[C@H]3N4C)c12]

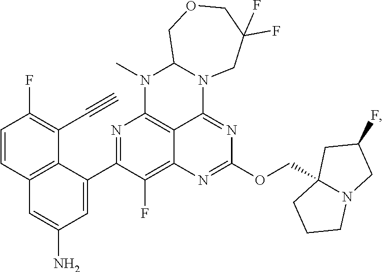 [SMILES: C#Cc1c(F)ccc2cc(N)cc(-c3nc4c5c(nc(OC[C@@]67CCCN6C[C@H](F)C7)nc5c3F)N3CC(F)(F)COCC3N4C)c12]

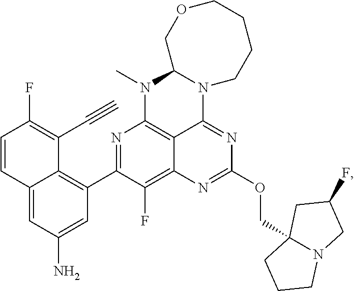 [SMILES: C#Cc1c(F)ccc2cc(N)cc(-c3nc4c5c(nc(OC[C@@]67CCCN6C[C@H](F)C7)nc5c3F)N3CCCCOC[C@@H]3N4C)c12]

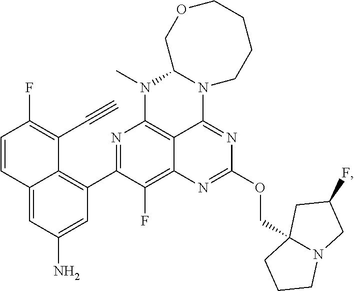 [SMILES: C#Cc1c(F)ccc2cc(N)cc(-c3nc4c5c(nc(OC[C@@]67CCCN6C[C@H](F)C7)nc5c3F)N3CCCCOC[C@H]3N4C)c12]

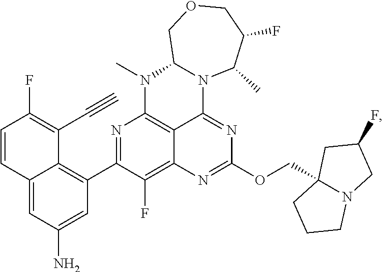 [SMILES: C#Cc1c(F)ccc2cc(N)cc(-c3nc4c5c(nc(OC[C@@]67CCCN6C[C@H](F)C7)nc5c3F)N3[C@@H](C)[C@@H](F)COC[C@H]3N4C)c12]

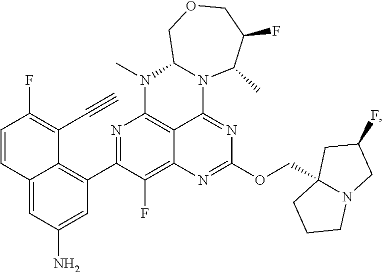 [SMILES: C#Cc1c(F)ccc2cc(N)cc(-c3nc4c5c(nc(OC[C@@]67CCCN6C[C@H](F)C7)nc5c3F)N3[C@@H](C)[C@H](F)COC[C@H]3N4C)c12]

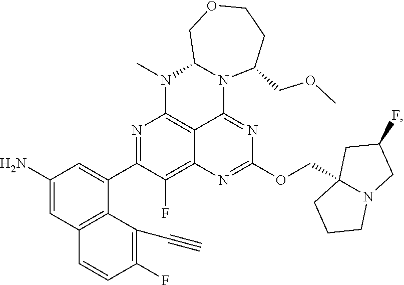 [SMILES: C#Cc1c(F)ccc2cc(N)cc(-c3nc4c5c(nc(OC[C@@]67CCCN6C[C@H](F)C7)nc5c3F)N3[C@@H](COC)CCOC[C@H]3N4C)c12]

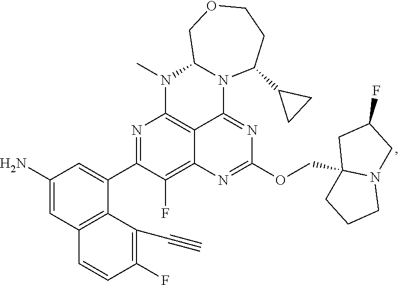 [SMILES: C#Cc1c(F)ccc2cc(N)cc(-c3nc4c5c(nc(OC[C@@]67CCCN6C[C@H](F)C7)nc5c3F)N3[C@@H](C5CC5)CCOC[C@H]3N4C)c12]

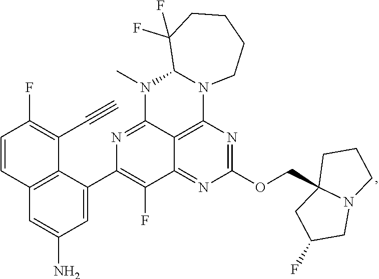 [SMILES: C#Cc1c(F)ccc2cc(N)cc(-c3nc4c5c(nc(OC[C@@]67CCCN6C[C@H](F)C7)nc5c3F)N3CCCCC(F)(F)[C@H]3N4C)c12]

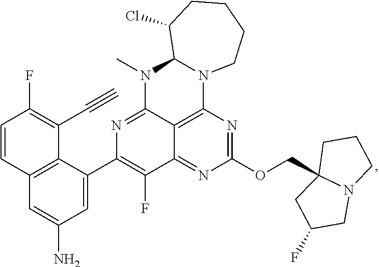 [SMILES: C#Cc1c(F)ccc2cc(N)cc(-c3nc4c5c(nc(OC[C@@]67CCCN6C[C@H](F)C7)nc5c3F)N3CCCC[C@@H](Cl)[C@@H]3N4C)c12]

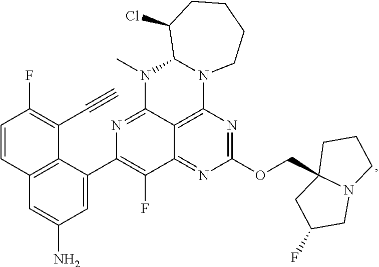 [SMILES: C#Cc1c(F)ccc2cc(N)cc(-c3nc4c5c(nc(OC[C@@]67CCCN6C[C@H](F)C7)nc5c3F)N3CCCC[C@H](Cl)[C@H]3N4C)c12]

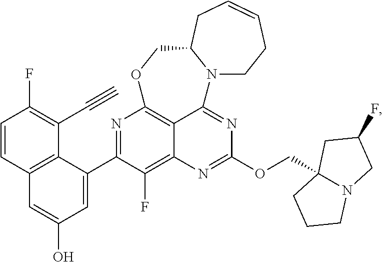 [SMILES: C#Cc1c(F)ccc2cc(O)cc(-c3nc4c5c(nc(OC[C@@]67CCCN6C[C@H](F)C7)nc5c3F)N3CCC=CC[C@H]3CO4)c12]

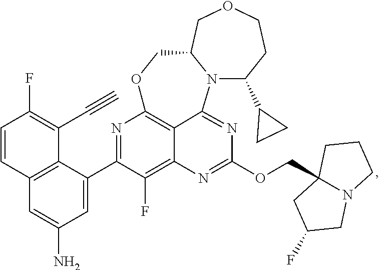 [SMILES: C#Cc1c(F)ccc2cc(N)cc(-c3nc4c5c(nc(OC[C@@]67CCCN6C[C@H](F)C7)nc5c3F)N3[C@@H](COCC[C@@H]3C3CC3)CO4)c12]

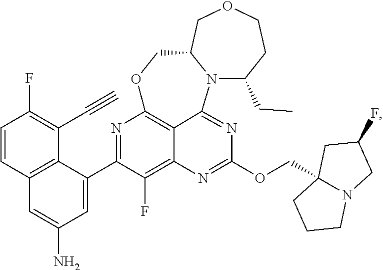 [SMILES: C#Cc1c(F)ccc2cc(N)cc(-c3nc4c5c(nc(OC[C@@]67CCCN6C[C@H](F)C7)nc5c3F)N3[C@@H](CC)CCOC[C@H]3CO4)c12]

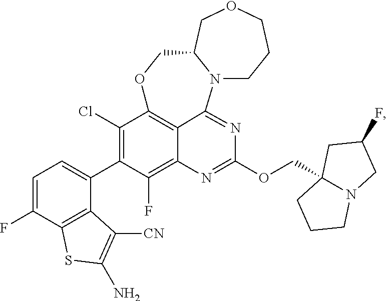 [SMILES: N#Cc1c(N)sc2c(F)ccc(-c3c(Cl)c4c5c(nc(OC[C@@]67CCCN6C[C@H](F)C7)nc5c3F)N3CCCOC[C@H]3CO4)c12]

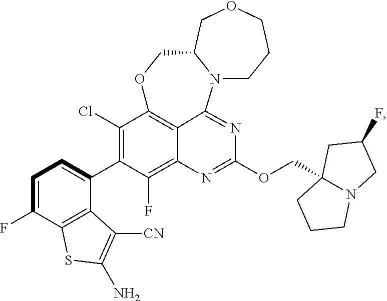

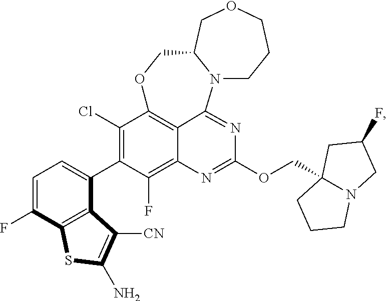

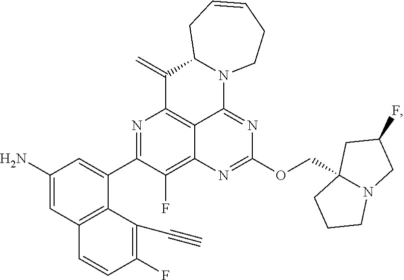 [SMILES: C#Cc1c(F)ccc2cc(N)cc(-c3nc4c5c(nc(OC[C@@]67CCCN6C[C@H](F)C7)nc5c3F)N3CCC=CC[C@H]3C4=C)c12]

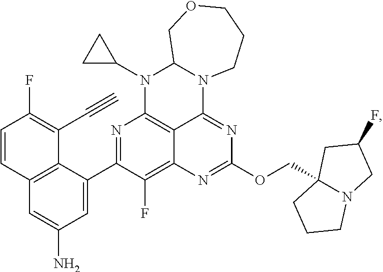 [SMILES: C#Cc1c(F)ccc2cc(N)cc(-c3nc4c5c(nc(OC[C@@]67CCCN6C[C@H](F)C7)nc5c3F)N3CCCOCC3N4C3CC3)c12]

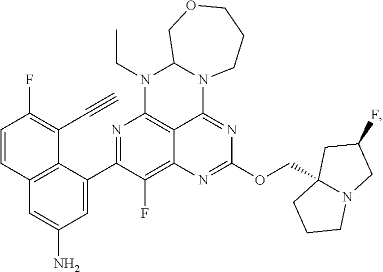 [SMILES: C#Cc1c(F)ccc2cc(N)cc(-c3nc4c5c(nc(OC[C@@]67CCCN6C[C@H](F)C7)nc5c3F)N3CCCOCC3N4CC)c12]

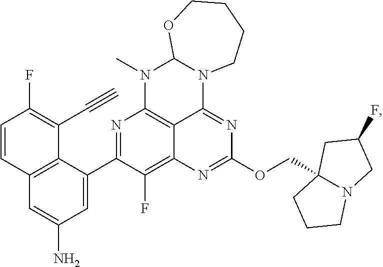 [SMILES: C#Cc1c(F)ccc2cc(N)cc(-c3nc4c5c(nc(OC[C@@]67CCCN6C[C@H](F)C7)nc5c3F)N3CCCCOC3N4C)c12]

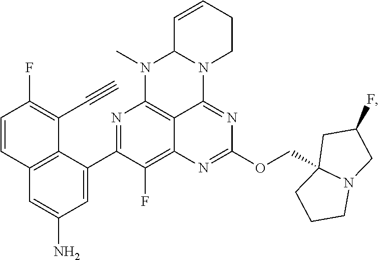 [SMILES: C#Cc1c(F)ccc2cc(N)cc(-c3nc4c5c(nc(OC[C@@]67CCCN6C[C@H](F)C7)nc5c3F)N3CCC=CC3N4C)c12]

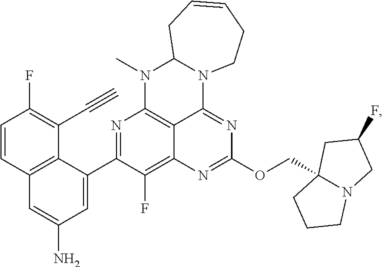 [SMILES: C#Cc1c(F)ccc2cc(N)cc(-c3nc4c5c(nc(OC[C@@]67CCCN6C[C@H](F)C7)nc5c3F)N3CCC=CCC3N4C)c12]

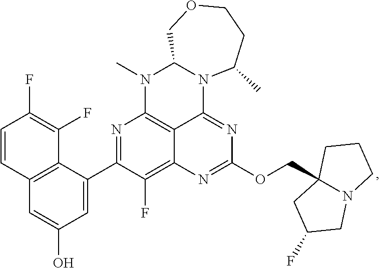 [SMILES: C[C@H]1CCOC[C@H]2N(C)c3nc(-c4cc(O)cc5ccc(F)c(F)c45)c(F)c4nc(OC[C@@]56CCCN5C[C@H](F)C6)nc(c34)N12]

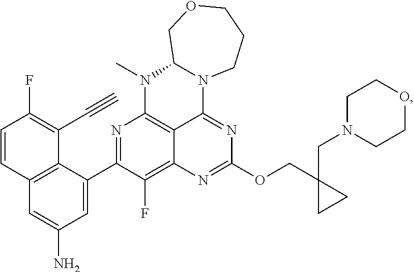 [SMILES: C#Cc1c(F)ccc2cc(N)cc(-c3nc4c5c(nc(OCC6(CN7CCOCC7)CC6)nc5c3F)N3CCCOC[C@H]3N4C)c12]

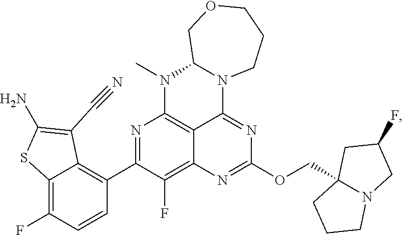 [SMILES: CN1c2nc(-c3ccc(F)c4sc(N)c(C#N)c34)c(F)c3nc(OC[C@@]45CCCN4C[C@H](F)C5)nc(c23)N2CCCOC[C@@H]12]

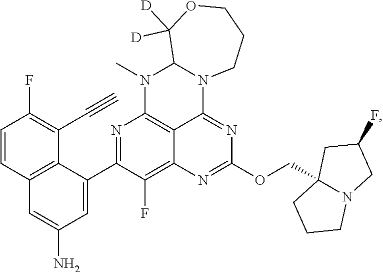 [SMILES: [2H]C1([2H])OCCCN2c3nc(OC[C@@]45CCCN4C[C@H](F)C5)nc4c(F)c(-c5cc(N)cc6ccc(F)c(C#C)c56)nc(c34)N(C)C21]

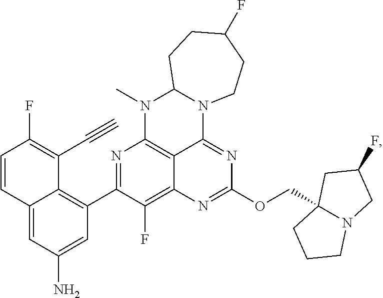 [SMILES: C#Cc1c(F)ccc2cc(N)cc(-c3nc4c5c(nc(OC[C@@]67CCCN6C[C@H](F)C7)nc5c3F)N3CCC(F)CCC3N4C)c12]

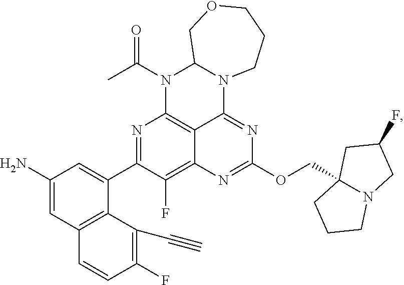 [SMILES: C#Cc1c(F)ccc2cc(N)cc(-c3nc4c5c(nc(OC[C@@]67CCCN6C[C@H](F)C7)nc5c3F)N3CCCOCC3N4C(C)=O)c12]

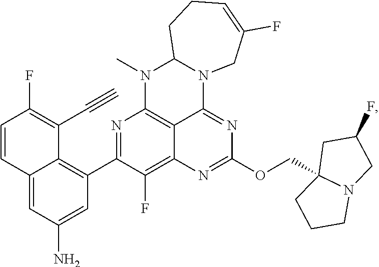 [SMILES: C#Cc1c(F)ccc2cc(N)cc(-c3nc4c5c(nc(OC[C@@]67CCCN6C[C@H](F)C7)nc5c3F)N3CC(F)=CCCC3N4C)c12]

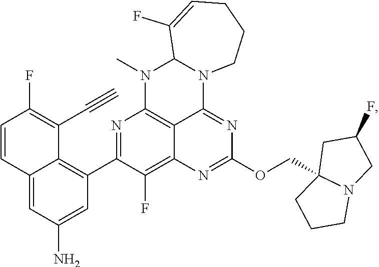 [SMILES: C#Cc1c(F)ccc2cc(N)cc(-c3nc4c5c(nc(OC[C@@]67CCCN6C[C@H](F)C7)nc5c3F)N3CCCC=C(F)C3N4C)c12]

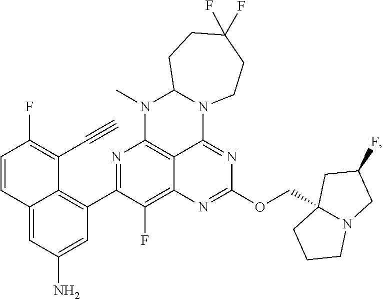 [SMILES: C#Cc1c(F)ccc2cc(N)cc(-c3nc4c5c(nc(OC[C@@]67CCCN6C[C@H](F)C7)nc5c3F)N3CCC(F)(F)CCC3N4C)c12]

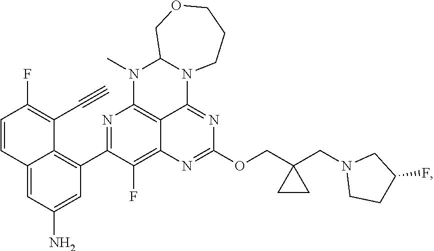 [SMILES: C#Cc1c(F)ccc2cc(N)cc(-c3nc4c5c(nc(OCC6(CN7CC[C@@H](F)C7)CC6)nc5c3F)N3CCCOCC3N4C)c12]

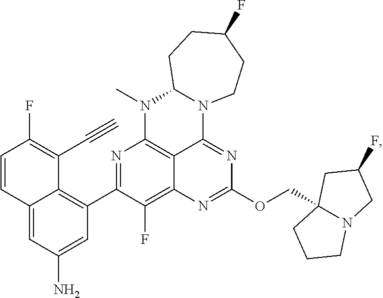 [SMILES: C#Cc1c(F)ccc2cc(N)cc(-c3nc4c5c(nc(OC[C@@]67CCCN6C[C@H](F)C7)nc5c3F)N3CC[C@H](F)CC[C@H]3N4C)c12]

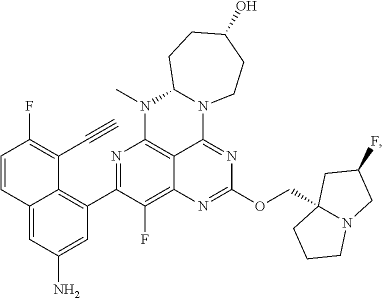 [SMILES: C#Cc1c(F)ccc2cc(N)cc(-c3nc4c5c(nc(OC[C@@]67CCCN6C[C@H](F)C7)nc5c3F)N3CC[C@@H](O)CC[C@H]3N4C)c12]

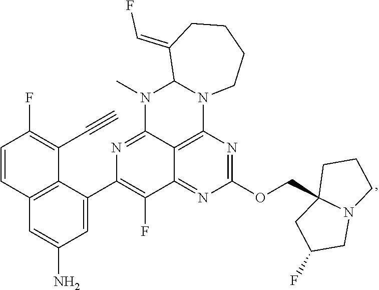 [SMILES: C#Cc1c(F)ccc2cc(N)cc(-c3nc4c5c(nc(OC[C@@]67CCCN6C[C@H](F)C7)nc5c3F)N3CCCC/C(=C\F)C3N4C)c12 ; ","]

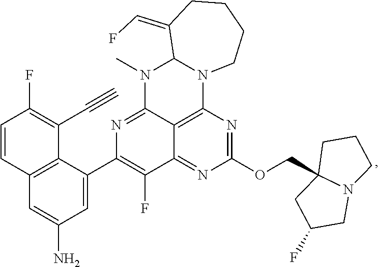 [SMILES: C#Cc1c(F)ccc2cc(N)cc(-c3nc4c5c(nc(OC[C@@]67CCCN6C[C@H](F)C7)nc5c3F)N3CCCC/C(=C/F)C3N4C)c12 ; ","]

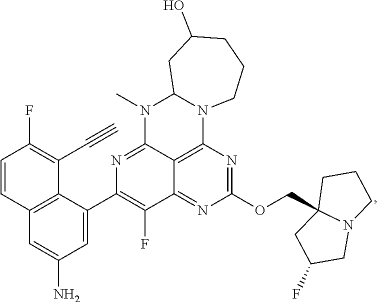 [SMILES: C#Cc1c(F)ccc2cc(N)cc(-c3nc4c5c(nc(OC[C@@]67CCCN6C[C@H](F)C7)nc5c3F)N3CCCC(O)CC3N4C)c12 ; ","]

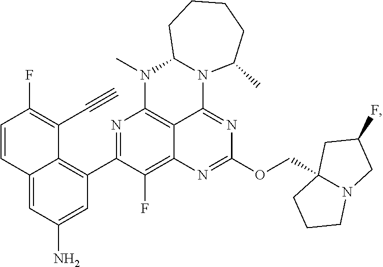 [SMILES: C#Cc1c(F)ccc2cc(N)cc(-c3nc4c5c(nc(OC[C@@]67CCCN6C[C@H](F)C7)nc5c3F)N3[C@@H](C)CCCC[C@H]3N4C)c12]

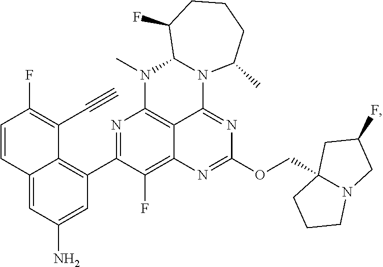 [SMILES: C#Cc1c(F)ccc2cc(N)cc(-c3nc4c5c(nc(OC[C@@]67CCCN6C[C@H](F)C7)nc5c3F)N3[C@@H](C)CCC[C@H](F)[C@H]3N4C)c12]

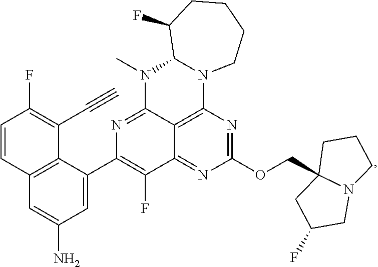 [SMILES: C#Cc1c(F)ccc2cc(N)cc(-c3nc4c5c(nc(OC[C@@]67CCCN6C[C@H](F)C7)nc5c3F)N3CCCC[C@H](F)[C@H]3N4C)c12 ; ","]

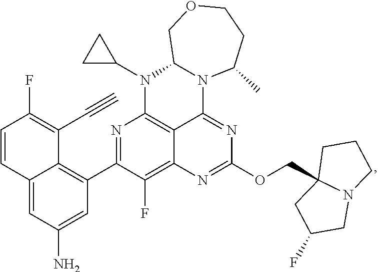 [SMILES: C#Cc1c(F)ccc2cc(N)cc(-c3nc4c5c(nc(OC[C@@]67CCCN6C[C@H](F)C7)nc5c3F)N3[C@@H](C)CCOC[C@H]3N4C3CC3)c12 ; ","]

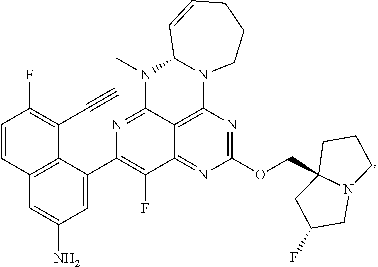 [SMILES: C#Cc1c(F)ccc2cc(N)cc(-c3nc4c5c(nc(OC[C@@]67CCCN6C[C@H](F)C7)nc5c3F)N3CCCC=C[C@H]3N4C)c12 ; ","]

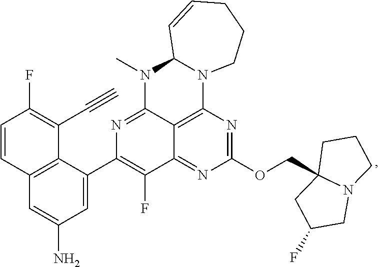 [SMILES: C#Cc1c(F)ccc2cc(N)cc(-c3nc4c5c(nc(OC[C@@]67CCCN6C[C@H](F)C7)nc5c3F)N3CCCC=C[C@@H]3N4C)c12 ; ","]

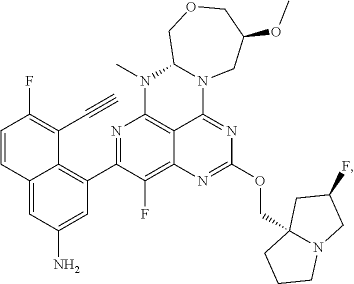 [SMILES: C#Cc1c(F)ccc2cc(N)cc(-c3nc4c5c(nc(OC[C@@]67CCCN6C[C@H](F)C7)nc5c3F)N3C[C@H](OC)COC[C@H]3N4C)c12]

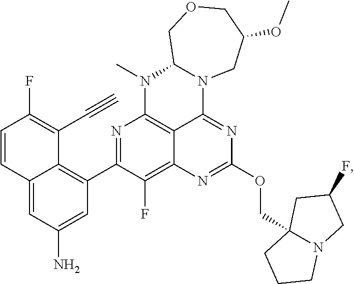 [SMILES: C#Cc1c(F)ccc2cc(N)cc(-c3nc4c5c(nc(OC[C@@]67CCCN6C[C@H](F)C7)nc5c3F)N3C[C@@H](OC)COC[C@H]3N4C)c12]

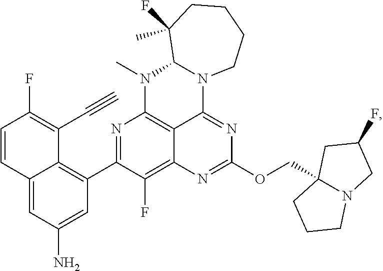 [SMILES: C#Cc1c(F)ccc2cc(N)cc(-c3nc4c5c(nc(OC[C@@]67CCCN6C[C@H](F)C7)nc5c3F)N3CCCC[C@](C)(F)[C@H]3N4C)c12]

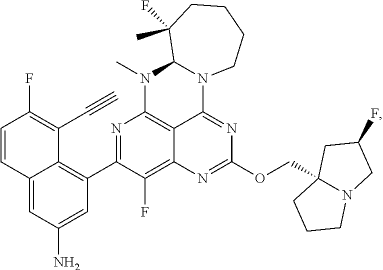 [SMILES: C#Cc1c(F)ccc2cc(N)cc(-c3nc4c5c(nc(OC[C@@]67CCCN6C[C@H](F)C7)nc5c3F)N3CCCC[C@@](C)(F)[C@@H]3N4C)c12]

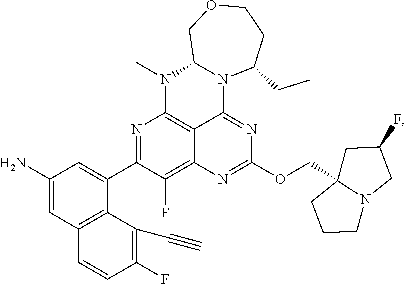 [SMILES: C#Cc1c(F)ccc2cc(N)cc(-c3nc4c5c(nc(OC[C@@]67CCCN6C[C@H](F)C7)nc5c3F)N3[C@@H](CC)CCOC[C@H]3N4C)c12]

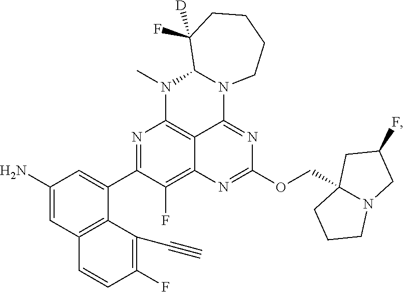 [SMILES: [2H][C@]1(F)CCCCN2c3nc(OC[C@@]45CCCN4C[C@H](F)C5)nc4c(F)c(-c5cc(N)cc6ccc(F)c(C#C)c56)nc(c34)N(C)[C@@H]21]

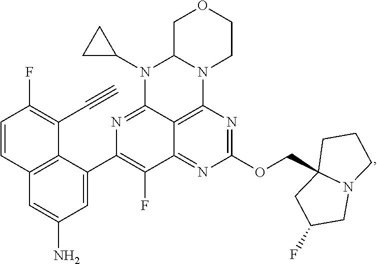 [SMILES: C#Cc1c(F)ccc2cc(N)cc(-c3nc4c5c(nc(OC[C@@]67CCCN6C[C@H](F)C7)nc5c3F)N3CCOCC3N4C3CC3)c12 ; ","]

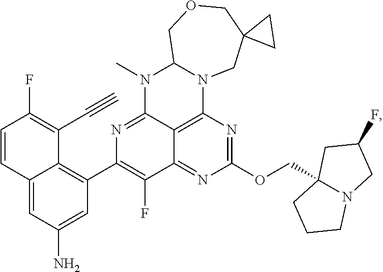 [SMILES: C#Cc1c(F)ccc2cc(N)cc(-c3nc4c5c(nc(OC[C@@]67CCCN6C[C@H](F)C7)nc5c3F)N3CC5(CC5)COCC3N4C)c12]

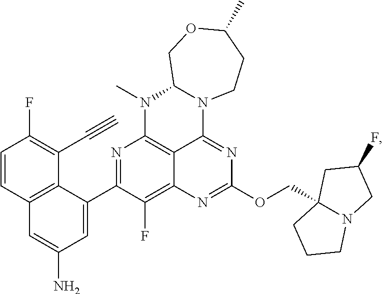 [SMILES: C#Cc1c(F)ccc2cc(N)cc(-c3nc4c5c(nc(OC[C@@]67CCCN6C[C@H](F)C7)nc5c3F)N3CC[C@@H](C)OC[C@H]3N4C)c12]

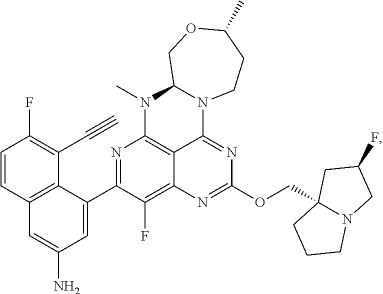 [SMILES: C#Cc1c(F)ccc2cc(N)cc(-c3nc4c5c(nc(OC[C@@]67CCCN6C[C@H](F)C7)nc5c3F)N3CC[C@@H](C)OC[C@@H]3N4C)c12]

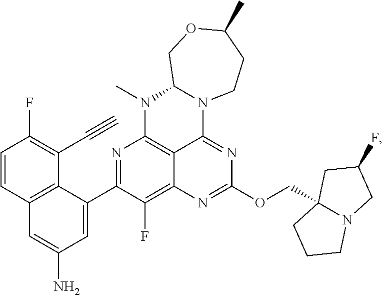 [SMILES: C#Cc1c(F)ccc2cc(N)cc(-c3nc4c5c(nc(OC[C@@]67CCCN6C[C@H](F)C7)nc5c3F)N3CC[C@H](C)OC[C@H]3N4C)c12]

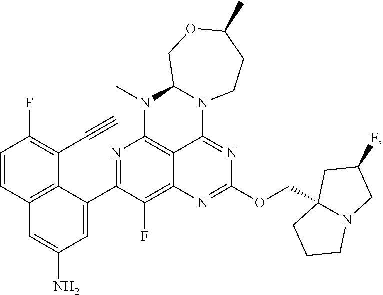 [SMILES: C#Cc1c(F)ccc2cc(N)cc(-c3nc4c5c(nc(OC[C@@]67CCCN6C[C@H](F)C7)nc5c3F)N3CC[C@H](C)OC[C@@H]3N4C)c12]

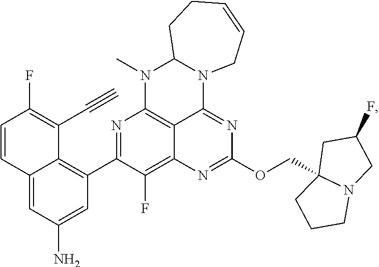 [SMILES: C#Cc1c(F)ccc2cc(N)cc(-c3nc4c5c(nc(OC[C@@]67CCCN6C[C@H](F)C7)nc5c3F)N3CC=CCCC3N4C)c12]

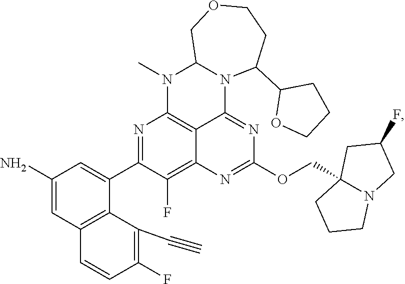 [SMILES: C#Cc1c(F)ccc2cc(N)cc(-c3nc4c5c(nc(OC[C@@]67CCCN6C[C@H](F)C7)nc5c3F)N3C(C5CCCO5)CCOCC3N4C)c12]

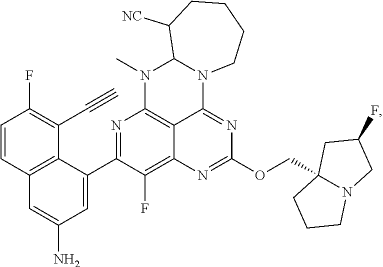 [SMILES: C#Cc1c(F)ccc2cc(N)cc(-c3nc4c5c(nc(OC[C@@]67CCCN6C[C@H](F)C7)nc5c3F)N3CCCCC(C#N)C3N4C)c12]

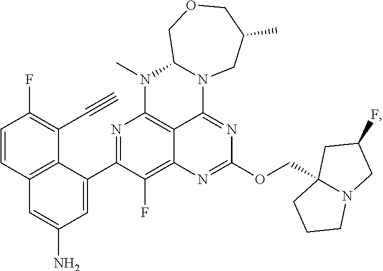 [SMILES: C#Cc1c(F)ccc2cc(N)cc(-c3nc4c5c(nc(OC[C@@]67CCCN6C[C@H](F)C7)nc5c3F)N3C[C@@H](C)COC[C@H]3N4C)c12]

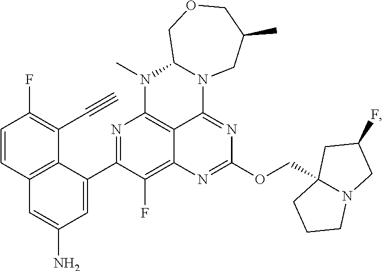 [SMILES: C#Cc1c(F)ccc2cc(N)cc(-c3nc4c5c(nc(OC[C@@]67CCCN6C[C@H](F)C7)nc5c3F)N3C[C@H](C)COC[C@H]3N4C)c12]

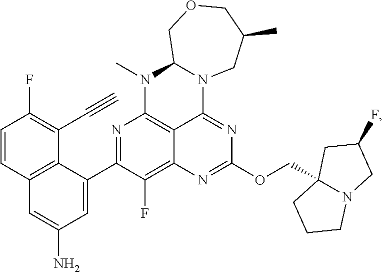 [SMILES: C#Cc1c(F)ccc2cc(N)cc(-c3nc4c5c(nc(OC[C@@]67CCCN6C[C@H](F)C7)nc5c3F)N3C[C@H](C)COC[C@@H]3N4C)c12]

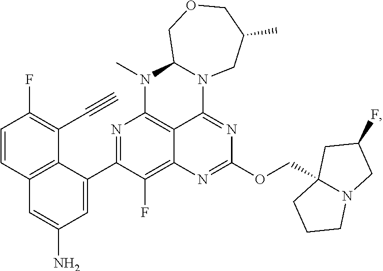 [SMILES: C#Cc1c(F)ccc2cc(N)cc(-c3nc4c5c(nc(OC[C@@]67CCCN6C[C@H](F)C7)nc5c3F)N3C[C@@H](C)COC[C@@H]3N4C)c12]

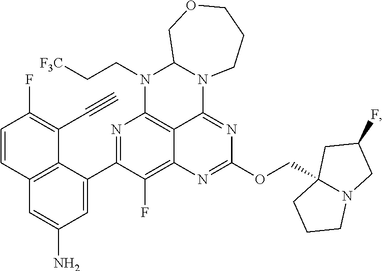 [SMILES: C#Cc1c(F)ccc2cc(N)cc(-c3nc4c5c(nc(OC[C@@]67CCCN6C[C@H](F)C7)nc5c3F)N3CCCOCC3N4CCC(F)(F)F)c12]

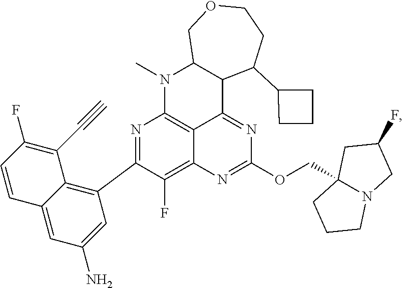 [SMILES: C#Cc1c(F)ccc2cc(N)cc(-c3nc4c5c(nc(OC[C@@]67CCCN6C[C@H](F)C7)nc5c3F)C3C(C5CCC5)CCOCC3N4C)c12]

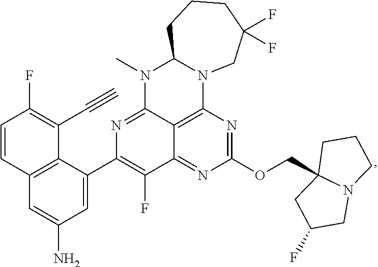 [SMILES: C#Cc1c(F)ccc2cc(N)cc(-c3nc4c5c(nc(OC[C@@]67CCCN6C[C@H](F)C7)nc5c3F)N3CC(F)(F)CCC[C@@H]3N4C)c12 ; ","]

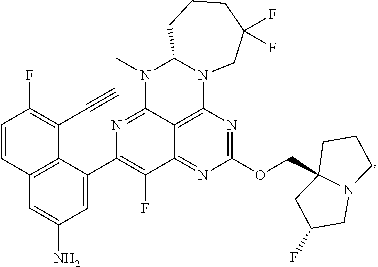 [SMILES: C#Cc1c(F)ccc2cc(N)cc(-c3nc4c5c(nc(OC[C@@]67CCCN6C[C@H](F)C7)nc5c3F)N3CC(F)(F)CCC[C@H]3N4C)c12 ; ","]

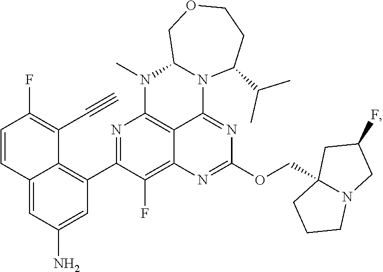 [SMILES: C#Cc1c(F)ccc2cc(N)cc(-c3nc4c5c(nc(OC[C@@]67CCCN6C[C@H](F)C7)nc5c3F)N3[C@@H](C(C)C)CCOC[C@H]3N4C)c12]

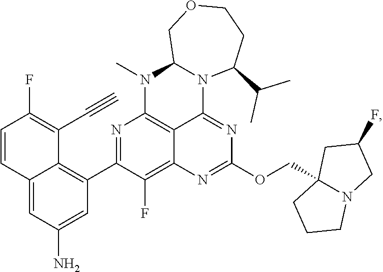 [SMILES: C#Cc1c(F)ccc2cc(N)cc(-c3nc4c5c(nc(OC[C@@]67CCCN6C[C@H](F)C7)nc5c3F)N3[C@H](COCC[C@H]3C(C)C)N4C)c12]

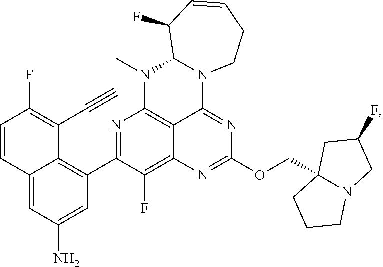 [SMILES: C#Cc1c(F)ccc2cc(N)cc(-c3nc4c5c(nc(OC[C@@]67CCCN6C[C@H](F)C7)nc5c3F)N3CCC=C[C@H](F)[C@H]3N4C)c12]

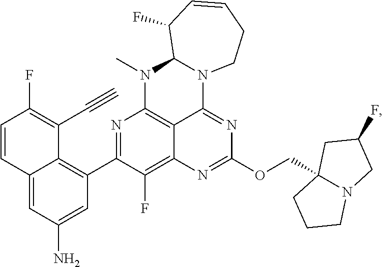 [SMILES: C#Cc1c(F)ccc2cc(N)cc(-c3nc4c5c(nc(OC[C@@]67CCCN6C[C@H](F)C7)nc5c3F)N3CCC=C[C@@H](F)[C@@H]3N4C)c12]

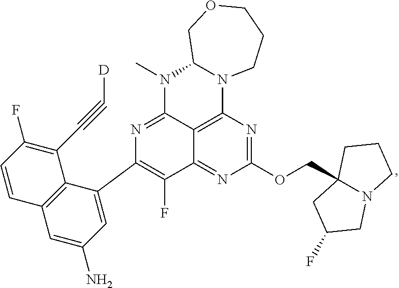 [SMILES: [2H]C#Cc1c(F)ccc2cc(N)cc(-c3nc4c5c(nc(OC[C@@]67CCCN6C[C@H](F)C7)nc5c3F)N3CCCOC[C@H]3N4C)c12 ; ","]

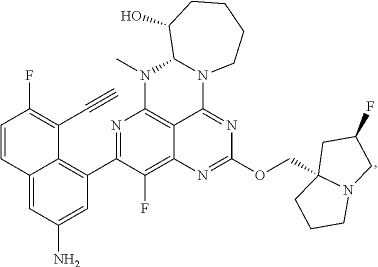 [SMILES: C#Cc1c(F)ccc2cc(N)cc(-c3nc4c5c(nc(OC[C@@]67CCCN6C[C@H](F)C7)nc5c3F)N3CCCC[C@@H](O)[C@H]3N4C)c12 ; ","]

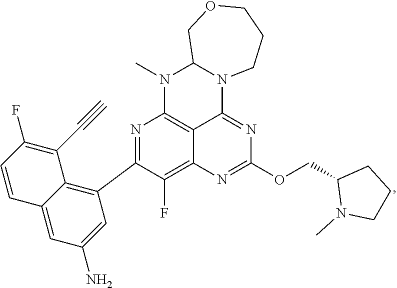 [SMILES: C#Cc1c(F)ccc2cc(N)cc(-c3nc4c5c(nc(OC[C@@H]6CCCN6C)nc5c3F)N3CCCOCC3N4C)c12 ; ","]

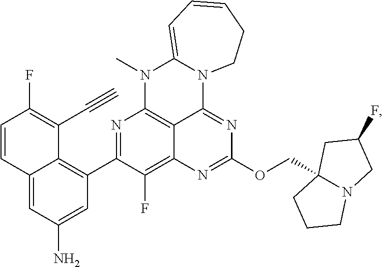 [SMILES: C#Cc1c(F)ccc2cc(N)cc(-c3nc4c5c(nc(OC[C@@]67CCCN6C[C@H](F)C7)nc5n5c(n4C)=CC=CCC5)c3F)c12]

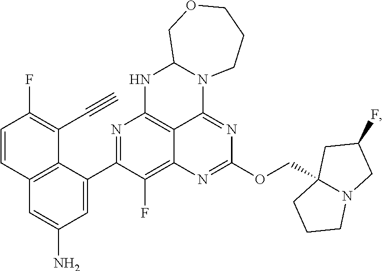 [SMILES: C#Cc1c(F)ccc2cc(N)cc(-c3nc4c5c(nc(OC[C@@]67CCCN6C[C@H](F)C7)nc5c3F)N3CCCOCC3N4)c12]

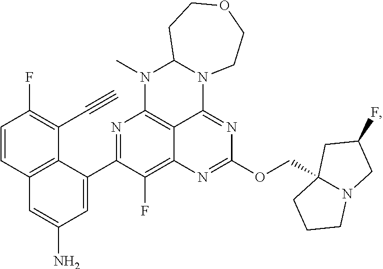 [SMILES: C#Cc1c(F)ccc2cc(N)cc(-c3nc4c5c(nc(OC[C@@]67CCCN6C[C@H](F)C7)nc5c3F)N3CCOCCC3N4C)c12]

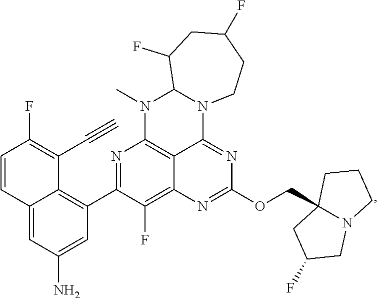 [SMILES: C#Cc1c(F)ccc2cc(N)cc(-c3nc4c5c(nc(OC[C@@]67CCCN6C[C@H](F)C7)nc5c3F)N3CCC(F)CC(F)C3N4C)c12 ; ","]

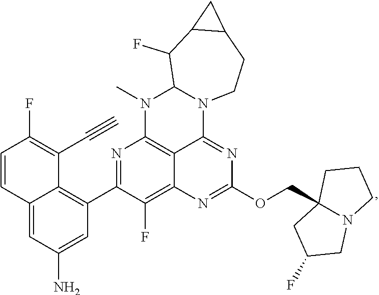 [SMILES: C#Cc1c(F)ccc2cc(N)cc(-c3nc4c5c(nc(OC[C@@]67CCCN6C[C@H](F)C7)nc5c3F)N3CCC5CC5C(F)C3N4C)c12 ; ","]

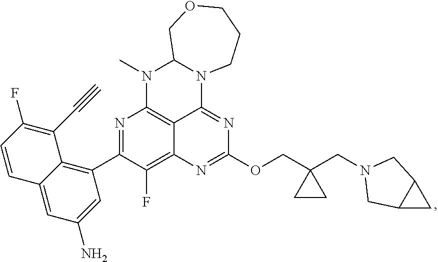 [SMILES: C#Cc1c(F)ccc2cc(N)cc(-c3nc4c5c(nc(OCC6(CN7CC8CC8C7)CC6)nc5c3F)N3CCCOCC3N4C)c12 ; ","]

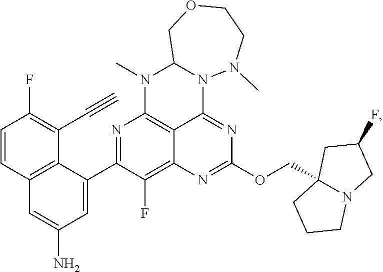 [SMILES: C#Cc1c(F)ccc2cc(N)cc(-c3nc4c5c(nc(OC[C@@]67CCCN6C[C@H](F)C7)nc5c3F)N3C(COCCN3C)N4C)c12]

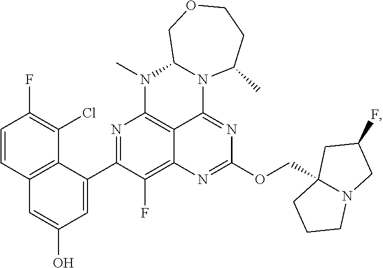 [SMILES: C[C@H]1CCOC[C@H]2N(C)c3nc(-c4cc(O)cc5ccc(F)c(Cl)c45)c(F)c4nc(OC[C@@]56CCCN5C[C@H](F)C6)nc(c34)N12]

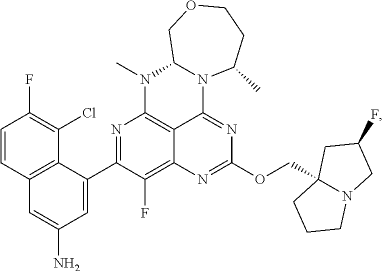 [SMILES: C[C@H]1CCOC[C@H]2N(C)c3nc(-c4cc(N)cc5ccc(F)c(Cl)c45)c(F)c4nc(OC[C@@]56CCCN5C[C@H](F)C6)nc(c34)N12]

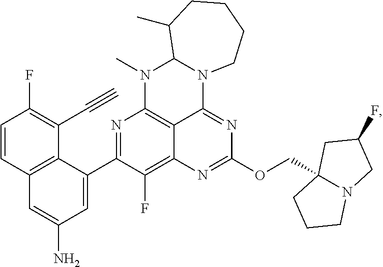 [SMILES: C#Cc1c(F)ccc2cc(N)cc(-c3nc4c5c(nc(OC[C@@]67CCCN6C[C@H](F)C7)nc5c3F)N3CCCCC(C)C3N4C)c12]

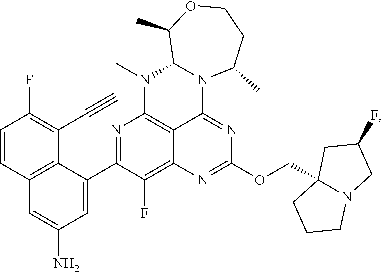 [SMILES: C#Cc1c(F)ccc2cc(N)cc(-c3nc4c5c(nc(OC[C@@]67CCCN6C[C@H](F)C7)nc5c3F)N3[C@@H](C)CCO[C@H](C)[C@H]3N4C)c12]

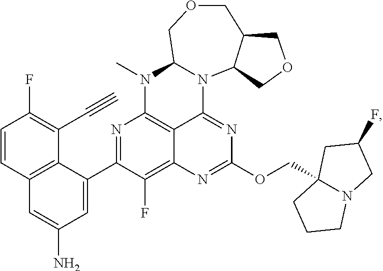 [SMILES: C#Cc1c(F)ccc2cc(N)cc(-c3nc4c5c(nc(OC[C@@]67CCCN6C[C@H](F)C7)nc5c3F)N3[C@@H]5COC[C@@H]5COC[C@@H]3N4C)c12]

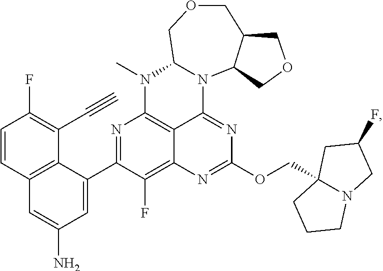 [SMILES: C#Cc1c(F)ccc2cc(N)cc(-c3nc4c5c(nc(OC[C@@]67CCCN6C[C@H](F)C7)nc5c3F)N3[C@@H]5COC[C@@H]5COC[C@H]3N4C)c12]

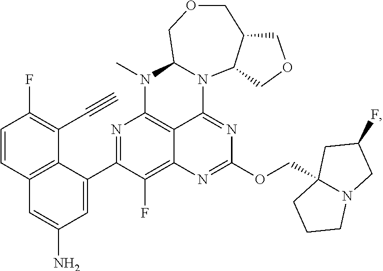 [SMILES: C#Cc1c(F)ccc2cc(N)cc(-c3nc4c5c(nc(OC[C@@]67CCCN6C[C@H](F)C7)nc5c3F)N3[C@H](COC[C@@H]5COC[C@@H]53)N4C)c12]

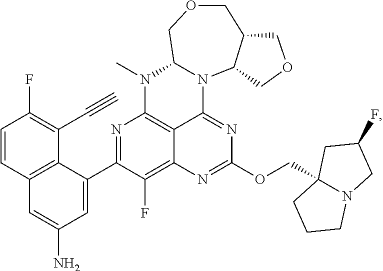 [SMILES: C#Cc1c(F)ccc2cc(N)cc(-c3nc4c5c(nc(OC[C@@]67CCCN6C[C@H](F)C7)nc5c3F)N3[C@H]5COC[C@H]5COC[C@H]3N4C)c12]

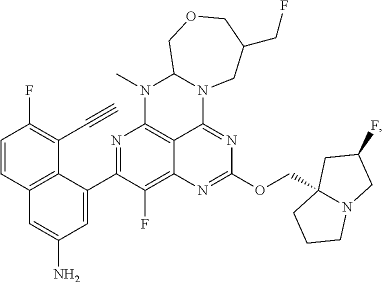 [SMILES: C#Cc1c(F)ccc2cc(N)cc(-c3nc4c5c(nc(OC[C@@]67CCCN6C[C@H](F)C7)nc5c3F)N3CC(CF)COCC3N4C)c12]

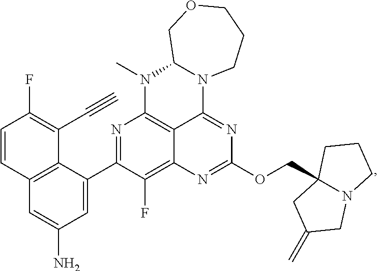 [SMILES: C#Cc1c(F)ccc2cc(N)cc(-c3nc4c5c(nc(OC[C@@]67CCCN6CC(=C)C7)nc5c3F)N3CCCOC[C@H]3N4C)c12 ; ","]

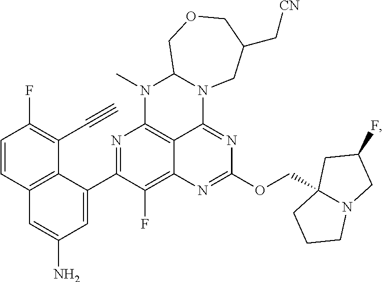 [SMILES: C#Cc1c(F)ccc2cc(N)cc(-c3nc4c5c(nc(OC[C@@]67CCCN6C[C@H](F)C7)nc5c3F)N3CC(CC#N)COCC3N4C)c12]

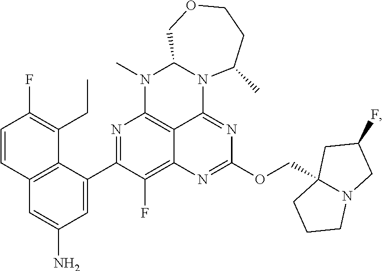 [SMILES: CCc1c(F)ccc2cc(N)cc(-c3nc4c5c(nc(OC[C@@]67CCCN6C[C@H](F)C7)nc5c3F)N3[C@@H](C)CCOC[C@H]3N4C)c12]

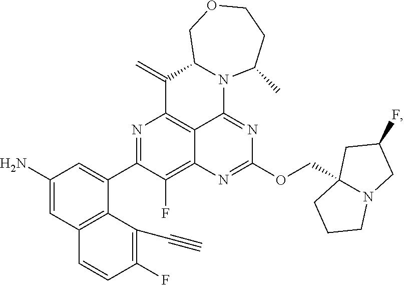 [SMILES: C#Cc1c(F)ccc2cc(N)cc(-c3nc4c5c(nc(OC[C@@]67CCCN6C[C@H](F)C7)nc5c3F)N3[C@@H](C)CCOC[C@H]3C4=C)c12]

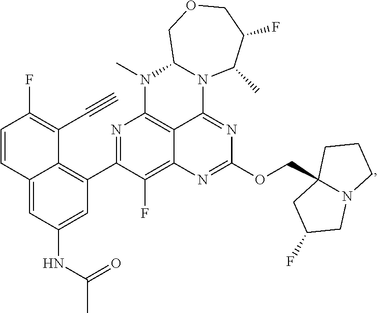 [SMILES: C#Cc1c(F)ccc2cc(NC(C)=O)cc(-c3nc4c5c(nc(OC[C@@]67CCCN6C[C@H](F)C7)nc5c3F)N3[C@@H](C)[C@@H](F)COC[C@H]3N4C)c12 ; ","]

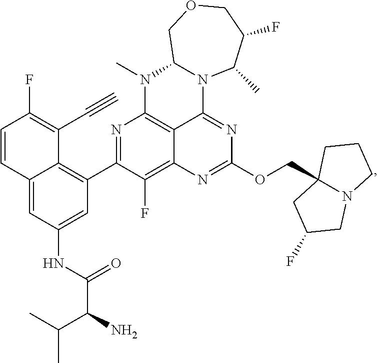 [SMILES: C#Cc1c(F)ccc2cc(NC(=O)[C@@H](N)C(C)C)cc(-c3nc4c5c(nc(OC[C@@]67CCCN6C[C@H](F)C7)nc5c3F)N3[C@@H](C)[C@@H](F)COC[C@H]3N4C)c12 ; ","]

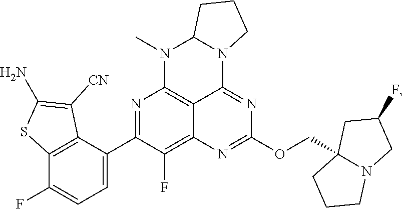 [SMILES: CN1c2nc(-c3ccc(F)c4sc(N)c(C#N)c34)c(F)c3nc(OC[C@@]45CCCN4C[C@H](F)C5)nc(c23)N2CCCC12]

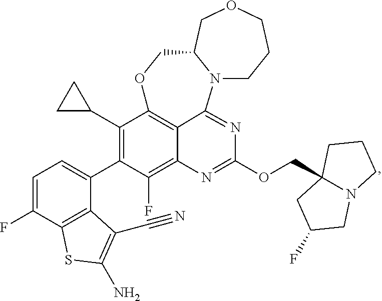 [SMILES: N#Cc1c(N)sc2c(F)ccc(-c3c(C4CC4)c4c5c(nc(OC[C@@]67CCCN6C[C@H](F)C7)nc5c3F)N3CCCOC[C@H]3CO4)c12 ; ","]

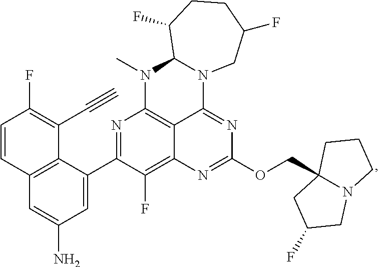 [SMILES: C#Cc1c(F)ccc2cc(N)cc(-c3nc4c5c(nc(OC[C@@]67CCCN6C[C@H](F)C7)nc5c3F)N3CC(F)CC[C@@H](F)[C@@H]3N4C)c12 ; ","]

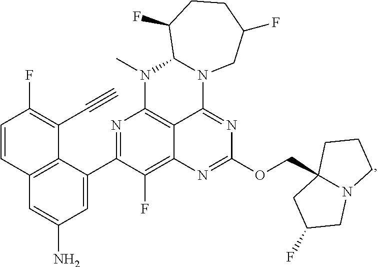 [SMILES: C#Cc1c(F)ccc2cc(N)cc(-c3nc4c5c(nc(OC[C@@]67CCCN6C[C@H](F)C7)nc5c3F)N3CC(F)CC[C@H](F)[C@H]3N4C)c12 ; ","]

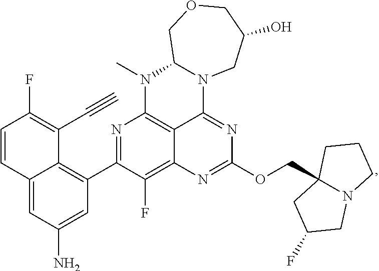 [SMILES: C#Cc1c(F)ccc2cc(N)cc(-c3nc4c5c(nc(OC[C@@]67CCCN6C[C@H](F)C7)nc5c3F)N3C[C@@H](O)COC[C@H]3N4C)c12 ; ","]

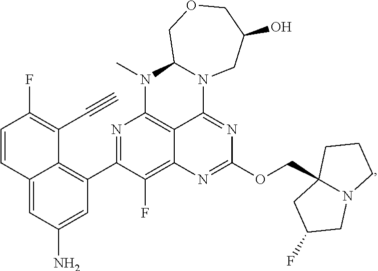 [SMILES: C#Cc1c(F)ccc2cc(N)cc(-c3nc4c5c(nc(OC[C@@]67CCCN6C[C@H](F)C7)nc5c3F)N3C[C@H](O)COC[C@@H]3N4C)c12 ; ","]

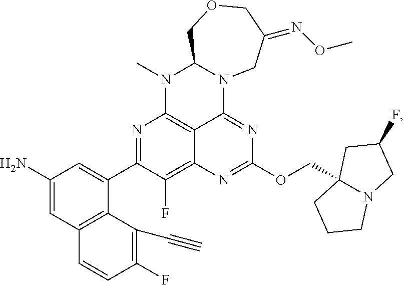 [SMILES: C#Cc1c(F)ccc2cc(N)cc(-c3nc4c5c(nc(OC[C@@]67CCCN6C[C@H](F)C7)nc5c3F)N3C/C(=N\OC)COC[C@@H]3N4C)c12]

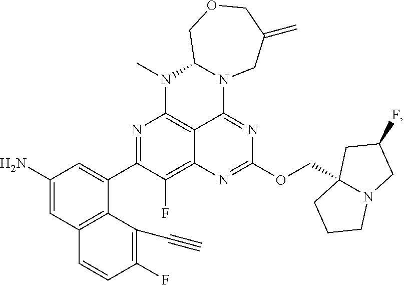 [SMILES: C#Cc1c(F)ccc2cc(N)cc(-c3nc4c5c(nc(OC[C@@]67CCCN6C[C@H](F)C7)nc5c3F)N3CC(=C)COC[C@H]3N4C)c12]

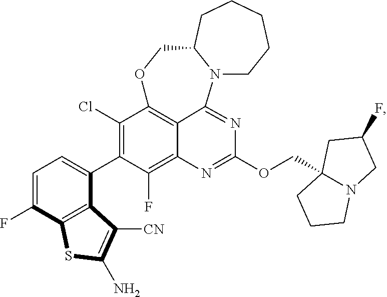

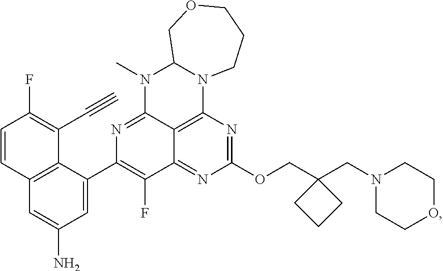 [SMILES: C#Cc1c(F)ccc2cc(N)cc(-c3nc4c5c(nc(OCC6(CN7CCOCC7)CCC6)nc5c3F)N3CCCOCC3N4C)c12]

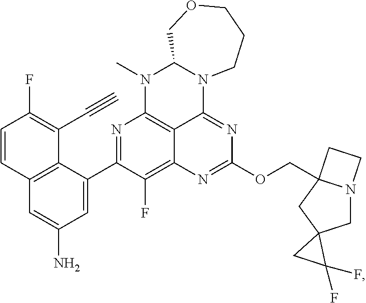 [SMILES: C#Cc1c(F)ccc2cc(N)cc(-c3nc4c5c(nc(OCC67CCN6CC6(C7)CC6(F)F)nc5c3F)N3CCCOC[C@H]3N4C)c12]

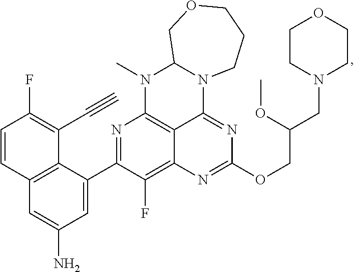 [SMILES: C#Cc1c(F)ccc2cc(N)cc(-c3nc4c5c(nc(OCC(CN6CCOCC6)OC)nc5c3F)N3CCCOCC3N4C)c12 ; ","]

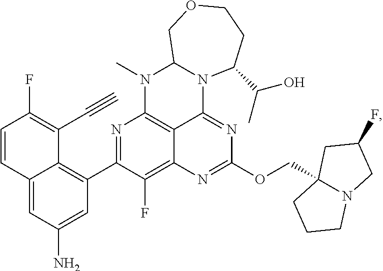 [SMILES: C#Cc1c(F)ccc2cc(N)cc(-c3nc4c5c(nc(OC[C@@]67CCCN6C[C@H](F)C7)nc5c3F)N3C(COCC[C@@H]3C(C)O)N4C)c12]

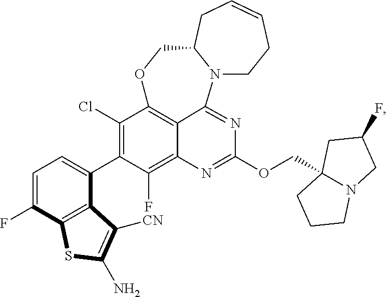

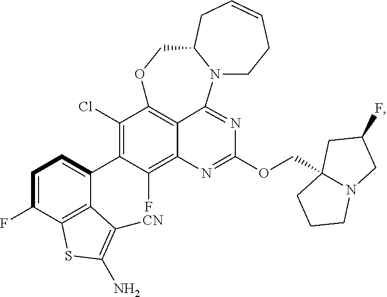

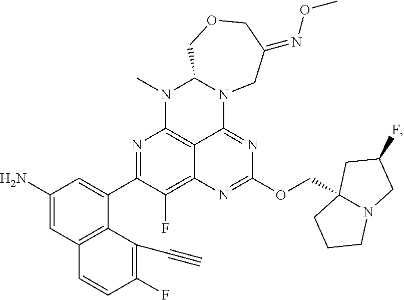 [SMILES: C#Cc1c(F)ccc2cc(N)cc(-c3nc4c5c(nc(OC[C@@]67CCCN6C[C@H](F)C7)nc5c3F)N3C/C(=N/OC)COC[C@H]3N4C)c12]

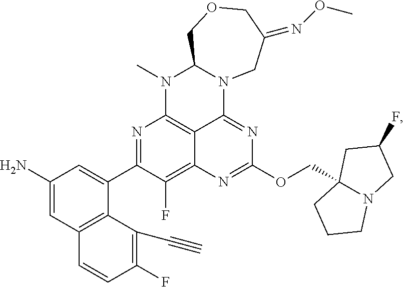 [SMILES: C#Cc1c(F)ccc2cc(N)cc(-c3nc4c5c(nc(OC[C@@]67CCCN6C[C@H](F)C7)nc5c3F)N3C/C(=N/OC)COC[C@@H]3N4C)c12]

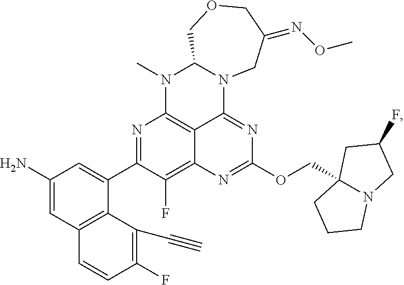 [SMILES: C#Cc1c(F)ccc2cc(N)cc(-c3nc4c5c(nc(OC[C@@]67CCCN6C[C@H](F)C7)nc5c3F)N3C/C(=N\OC)COC[C@H]3N4C)c12]

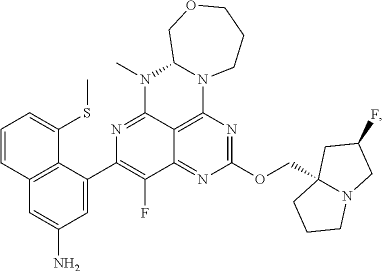 [SMILES: CSc1cccc2cc(N)cc(-c3nc4c5c(nc(OC[C@@]67CCCN6C[C@H](F)C7)nc5c3F)N3CCCOC[C@H]3N4C)c12]

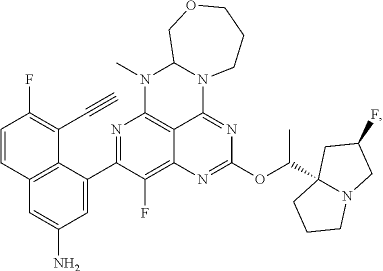 [SMILES: C#Cc1c(F)ccc2cc(N)cc(-c3nc4c5c(nc(OC(C)[C@@]67CCCN6C[C@H](F)C7)nc5c3F)N3CCCOCC3N4C)c12]

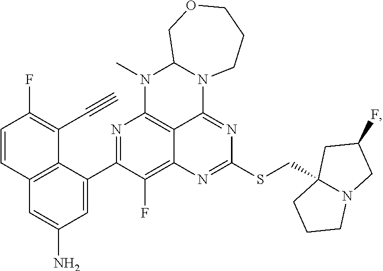 [SMILES: C#Cc1c(F)ccc2cc(N)cc(-c3nc4c5c(nc(SC[C@@]67CCCN6C[C@H](F)C7)nc5c3F)N3CCCOCC3N4C)c12]

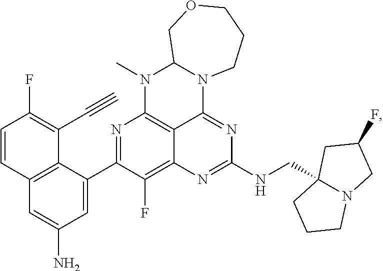 [SMILES: C#Cc1c(F)ccc2cc(N)cc(-c3nc4c5c(nc(NC[C@@]67CCCN6C[C@H](F)C7)nc5c3F)N3CCCOCC3N4C)c12]

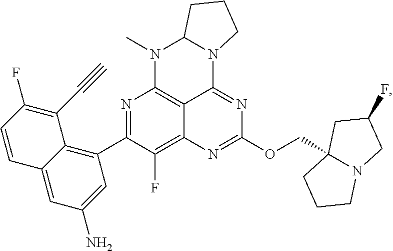 [SMILES: C#Cc1c(F)ccc2cc(N)cc(-c3nc4c5c(nc(OC[C@@]67CCCN6C[C@H](F)C7)nc5c3F)N3CCCC3N4C)c12]

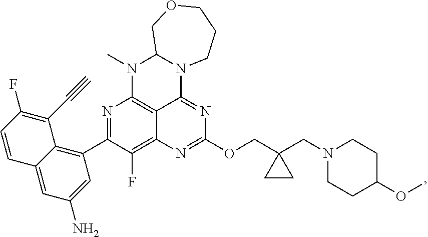

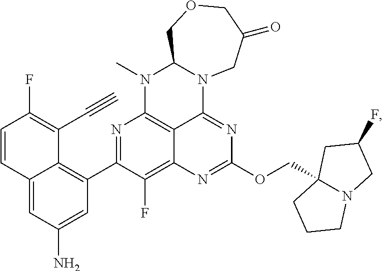 [SMILES: C#Cc1c(F)ccc2cc(N)cc(-c3nc4c5c(nc(OC[C@@]67CCCN6C[C@H](F)C7)nc5c3F)N3CC(=O)COC[C@@H]3N4C)c12]

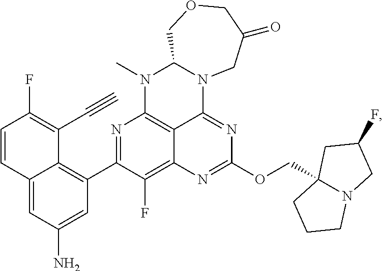 [SMILES: C#Cc1c(F)ccc2cc(N)cc(-c3nc4c5c(nc(OC[C@@]67CCCN6C[C@H](F)C7)nc5c3F)N3CC(=O)COC[C@H]3N4C)c12]

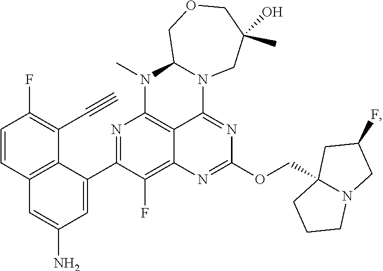 [SMILES: C#Cc1c(F)ccc2cc(N)cc(-c3nc4c5c(nc(OC[C@@]67CCCN6C[C@H](F)C7)nc5c3F)N3C[C@@](C)(O)COC[C@@H]3N4C)c12]

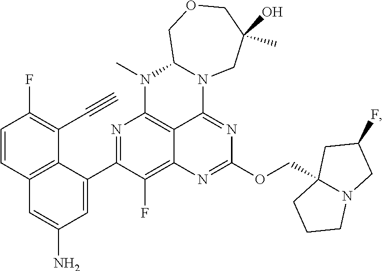 [SMILES: C#Cc1c(F)ccc2cc(N)cc(-c3nc4c5c(nc(OC[C@@]67CCCN6C[C@H](F)C7)nc5c3F)N3C[C@](C)(O)COC[C@H]3N4C)c12]

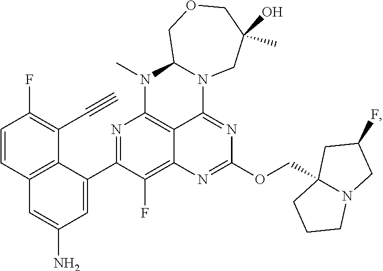 [SMILES: C#Cc1c(F)ccc2cc(N)cc(-c3nc4c5c(nc(OC[C@@]67CCCN6C[C@H](F)C7)nc5c3F)N3C[C@](C)(O)COC[C@@H]3N4C)c12]

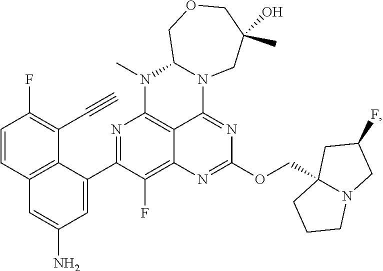 [SMILES: C#Cc1c(F)ccc2cc(N)cc(-c3nc4c5c(nc(OC[C@@]67CCCN6C[C@H](F)C7)nc5c3F)N3C[C@@](C)(O)COC[C@H]3N4C)c12]

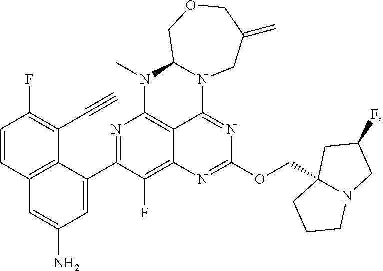 [SMILES: C#Cc1c(F)ccc2cc(N)cc(-c3nc4c5c(nc(OC[C@@]67CCCN6C[C@H](F)C7)nc5c3F)N3CC(=C)COC[C@@H]3N4C)c12]

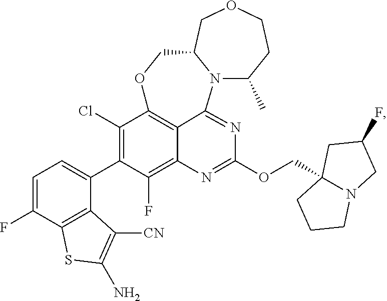 [SMILES: C[C@H]1CCOC[C@H]2COc3c(Cl)c(-c4ccc(F)c5sc(N)c(C#N)c45)c(F)c4nc(OC[C@@]56CCCN5C[C@H](F)C6)nc(c34)N21]

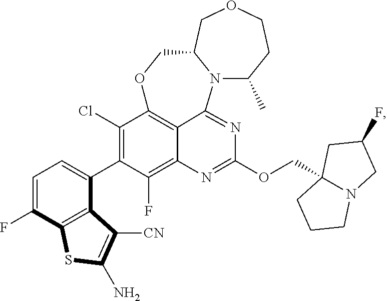

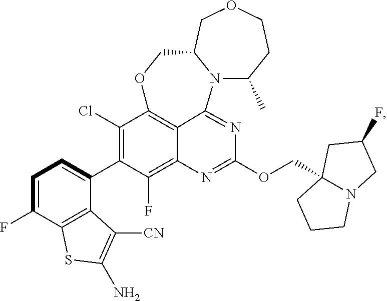

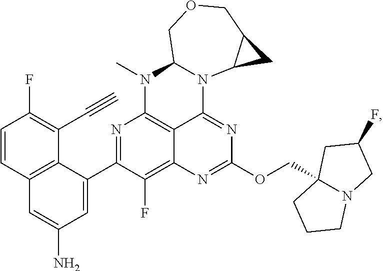 [SMILES: C#Cc1c(F)ccc2cc(N)cc(-c3nc4c5c(nc(OC[C@@]67CCCN6C[C@H](F)C7)nc5c3F)N3[C@@H]5C[C@@H]5COC[C@@H]3N4C)c12]

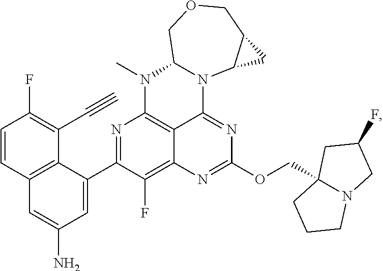 [SMILES: C#Cc1c(F)ccc2cc(N)cc(-c3nc4c5c(nc(OC[C@@]67CCCN6C[C@H](F)C7)nc5c3F)N3[C@H]5C[C@H]5COC[C@H]3N4C)c12]

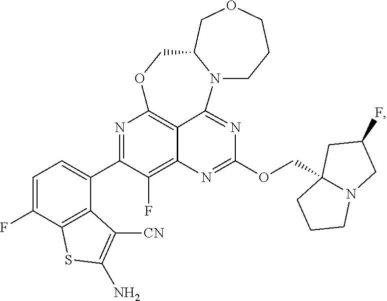 [SMILES: N#Cc1c(N)sc2c(F)ccc(-c3nc4c5c(nc(OC[C@@]67CCCN6C[C@H](F)C7)nc5c3F)N3CCCOC[C@H]3CO4)c12]

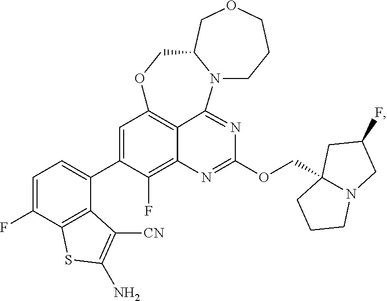 [SMILES: N#Cc1c(N)sc2c(F)ccc(-c3cc4c5c(nc(OC[C@@]67CCCN6C[C@H](F)C7)nc5c3F)N3CCCOC[C@H]3CO4)c12]

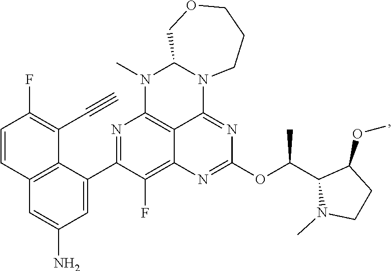

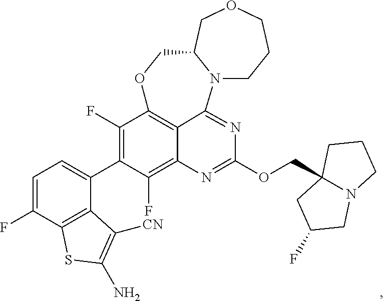 [SMILES: N#Cc1c(N)sc2c(F)ccc(-c3c(F)c4c5c(nc(OC[C@@]67CCCN6C[C@H](F)C7)nc5c3F)N3CCCOC[C@H]3CO4)c12]

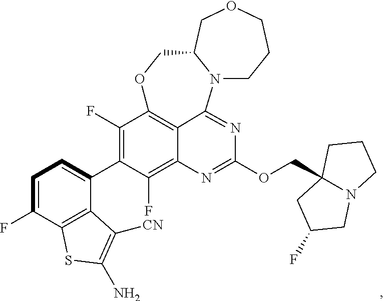

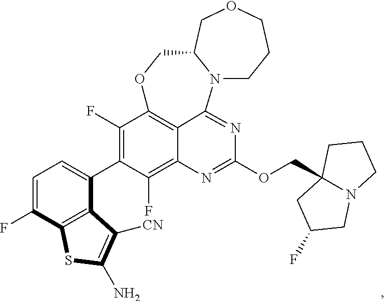

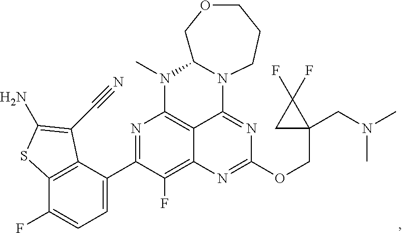 [SMILES: CN(C)CC1(COc2nc3c4c(nc(-c5ccc(F)c6sc(N)c(C#N)c56)c(F)c4n2)N(C)[C@@H]2COCCCN32)CC1(F)F]

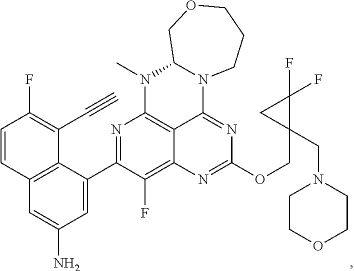 [SMILES: C#Cc1c(F)ccc2cc(N)cc(-c3nc4c5c(nc(OCC6(CN7CCOCC7)CC6(F)F)nc5c3F)N3CCCOC[C@H]3N4C)c12]

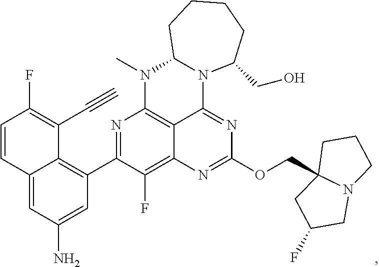 [SMILES: C#Cc1c(F)ccc2cc(N)cc(-c3nc4c5c(nc(OC[C@@]67CCCN6C[C@H](F)C7)nc5c3F)N3[C@@H](CO)CCCC[C@H]3N4C)c12]

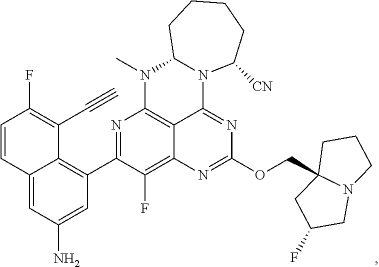 [SMILES: C#Cc1c(F)ccc2cc(N)cc(-c3nc4c5c(nc(OC[C@@]67CCCN6C[C@H](F)C7)nc5c3F)N3[C@@H](C#N)CCCC[C@H]3N4C)c12]

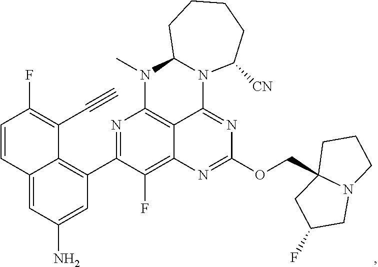 [SMILES: C#Cc1c(F)ccc2cc(N)cc(-c3nc4c5c(nc(OC[C@@]67CCCN6C[C@H](F)C7)nc5c3F)N3[C@H](CCCC[C@@H]3C#N)N4C)c12]

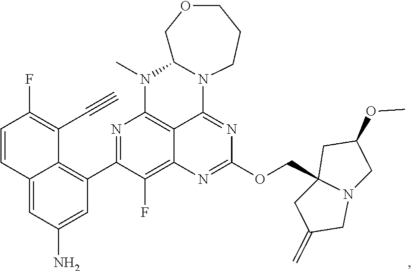 [SMILES: C#Cc1c(F)ccc2cc(N)cc(-c3nc4c5c(nc(OC[C@]67CC(=C)CN6C[C@H](OC)C7)nc5c3F)N3CCCOC[C@H]3N4C)c12]

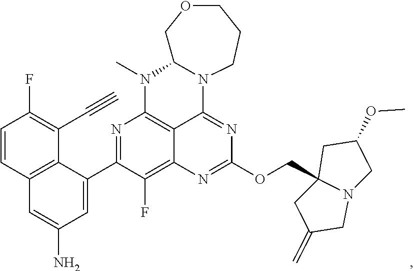 [SMILES: C#Cc1c(F)ccc2cc(N)cc(-c3nc4c5c(nc(OC[C@]67CC(=C)CN6C[C@@H](OC)C7)nc5c3F)N3CCCOC[C@H]3N4C)c12]

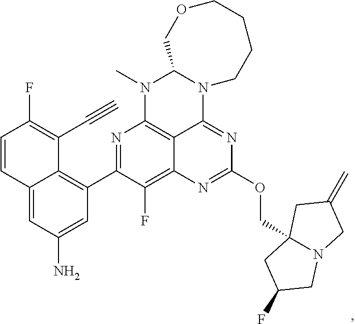 [SMILES: C#Cc1c(F)ccc2cc(N)cc(-c3nc4c5c(nc(OC[C@]67CC(=C)CN6C[C@@H](F)C7)nc5c3F)N3CCCCOC[C@H]3N4C)c12]

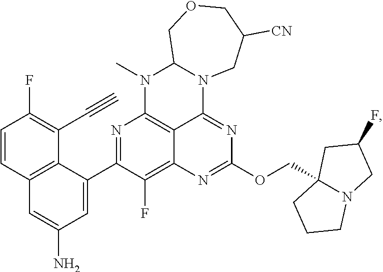 [SMILES: C#Cc1c(F)ccc2cc(N)cc(-c3nc4c5c(nc(OC[C@@]67CCCN6C[C@H](F)C7)nc5c3F)N3CC(C#N)COCC3N4C)c12]

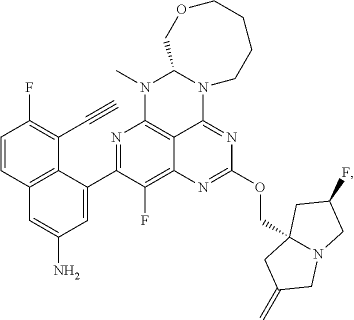 [SMILES: C#Cc1c(F)ccc2cc(N)cc(-c3nc4c5c(nc(OC[C@@]67CC(=C)CN6C[C@H](F)C7)nc5c3F)N3CCCCOC[C@H]3N4C)c12]

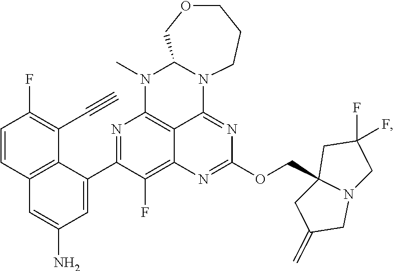 [SMILES: C#Cc1c(F)ccc2cc(N)cc(-c3nc4c5c(nc(OC[C@]67CC(=C)CN6CC(F)(F)C7)nc5c3F)N3CCCOC[C@H]3N4C)c12]

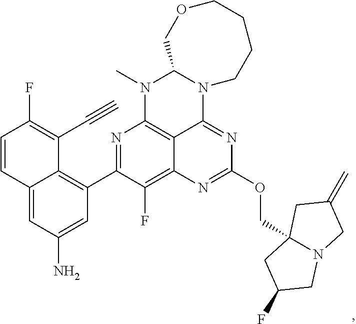 [SMILES: C#Cc1c(F)ccc2cc(N)cc(-c3nc4c5c(nc(OC[C@]67CC(=C)CN6C[C@@H](F)C7)nc5c3F)N3CCCCOC[C@H]3N4C)c12]

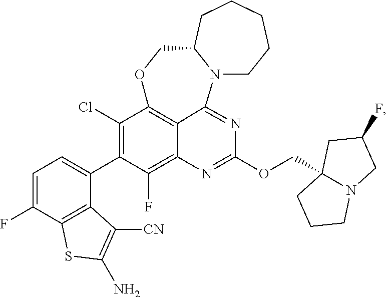 [SMILES: N#Cc1c(N)sc2c(F)ccc(-c3c(Cl)c4c5c(nc(OC[C@@]67CCCN6C[C@H](F)C7)nc5c3F)N3CCCCC[C@H]3CO4)c12]

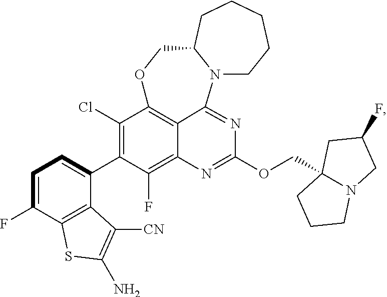

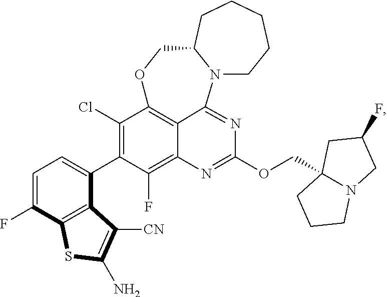

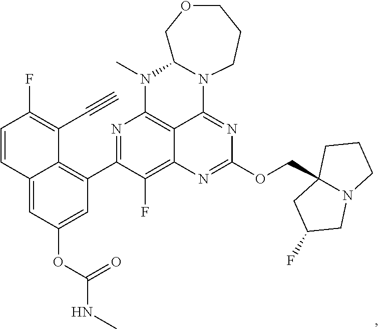 [SMILES: C#Cc1c(F)ccc2cc(OC(=O)NC)cc(-c3nc4c5c(nc(OC[C@@]67CCCN6C[C@H](F)C7)nc5c3F)N3CCCOC[C@H]3N4C)c12]

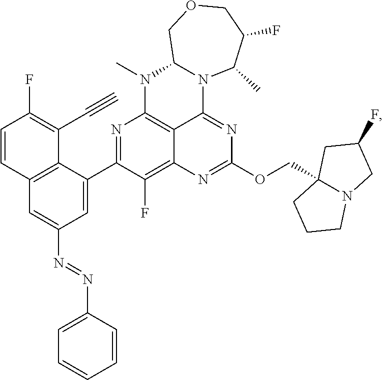 [SMILES: C#Cc1c(F)ccc2cc(/N=N/c3ccccc3)cc(-c3nc4c5c(nc(OC[C@@]67CCCN6C[C@H](F)C7)nc5c3F)N3[C@@H](C)[C@@H](F)COC[C@H]3N4C)c12]

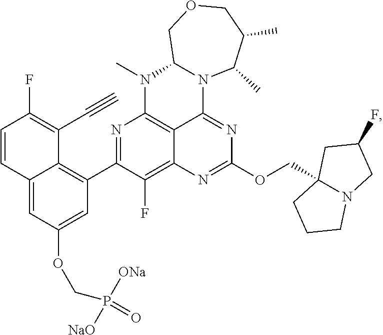

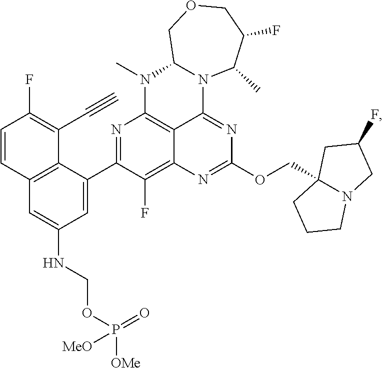 [SMILES: C#Cc1c(F)ccc2cc(NCOP(=O)(OC)OC)cc(-c3nc4c5c(nc(OC[C@@]67CCCN6C[C@H](F)C7)nc5c3F)N3[C@@H](C)[C@@H](F)COC[C@H]3N4C)c12]

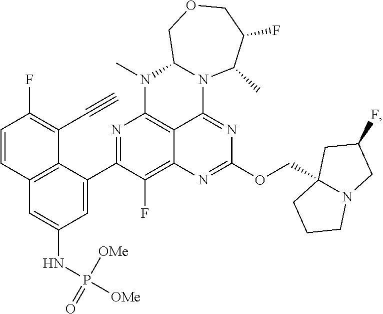 [SMILES: C#Cc1c(F)ccc2cc(NP(=O)(OC)OC)cc(-c3nc4c5c(nc(OC[C@@]67CCCN6C[C@H](F)C7)nc5c3F)N3[C@@H](C)[C@@H](F)COC[C@H]3N4C)c12]

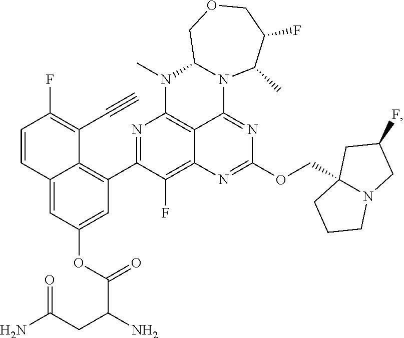 [SMILES: C#Cc1c(F)ccc2cc(OC(=O)C(N)CC(N)=O)cc(-c3nc4c5c(nc(OC[C@@]67CCCN6C[C@H](F)C7)nc5c3F)N3[C@@H](C)[C@@H](F)COC[C@H]3N4C)c12]

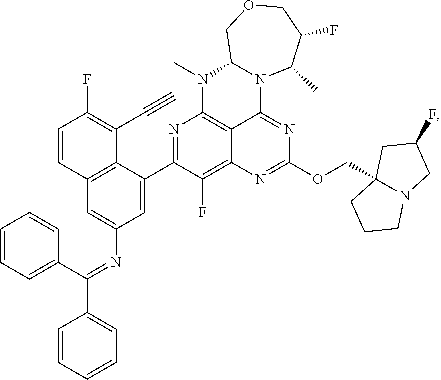 [SMILES: C#Cc1c(F)ccc2cc(N=C(c3ccccc3)c3ccccc3)cc(-c3nc4c5c(nc(OC[C@@]67CCCN6C[C@H](F)C7)nc5c3F)N3[C@@H](C)[C@@H](F)COC[C@H]3N4C)c12]

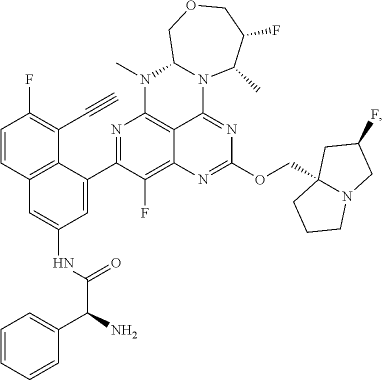 [SMILES: C#Cc1c(F)ccc2cc(NC(=O)[C@@H](N)c3ccccc3)cc(-c3nc4c5c(nc(OC[C@@]67CCCN6C[C@H](F)C7)nc5c3F)N3[C@@H](C)[C@@H](F)COC[C@H]3N4C)c12]

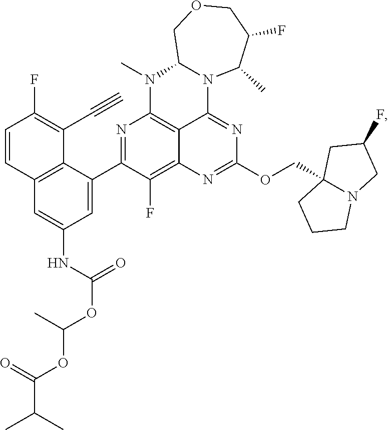 [SMILES: C#Cc1c(F)ccc2cc(NC(=O)OC(C)OC(=O)C(C)C)cc(-c3nc4c5c(nc(OC[C@@]67CCCN6C[C@H](F)C7)nc5c3F)N3[C@@H](C)[C@@H](F)COC[C@H]3N4C)c12]

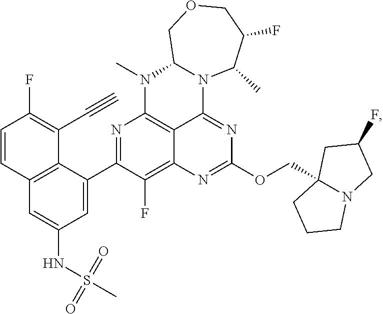 [SMILES: C#Cc1c(F)ccc2cc(NS(C)(=O)=O)cc(-c3nc4c5c(nc(OC[C@@]67CCCN6C[C@H](F)C7)nc5c3F)N3[C@@H](C)[C@@H](F)COC[C@H]3N4C)c12]

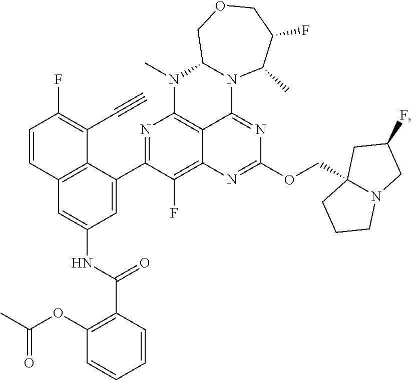 [SMILES: C#Cc1c(F)ccc2cc(NC(=O)c3ccccc3OC(C)=O)cc(-c3nc4c5c(nc(OC[C@@]67CCCN6C[C@H](F)C7)nc5c3F)N3[C@@H](C)[C@@H](F)COC[C@H]3N4C)c12]

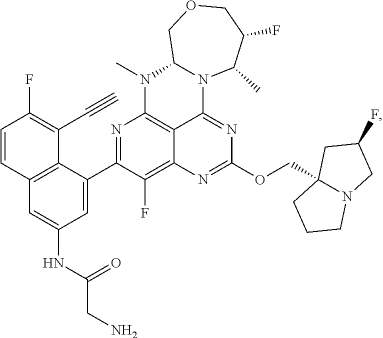 [SMILES: C#Cc1c(F)ccc2cc(NC(=O)CN)cc(-c3nc4c5c(nc(OC[C@@]67CCCN6C[C@H](F)C7)nc5c3F)N3[C@@H](C)[C@@H](F)COC[C@H]3N4C)c12]

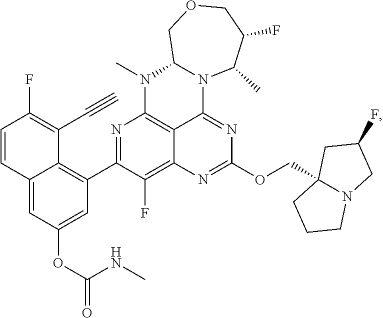 [SMILES: C#Cc1c(F)ccc2cc(OC(=O)NC)cc(-c3nc4c5c(nc(OC[C@@]67CCCN6C[C@H](F)C7)nc5c3F)N3[C@@H](C)[C@@H](F)COC[C@H]3N4C)c12]

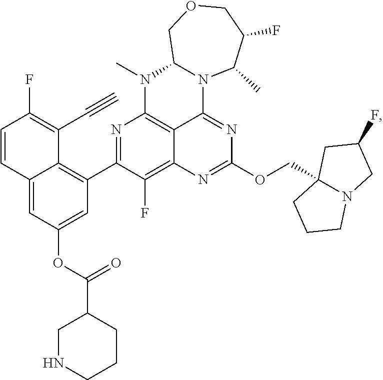 [SMILES: C#Cc1c(F)ccc2cc(OC(=O)C3CCCNC3)cc(-c3nc4c5c(nc(OC[C@@]67CCCN6C[C@H](F)C7)nc5c3F)N3[C@@H](C)[C@@H](F)COC[C@H]3N4C)c12]

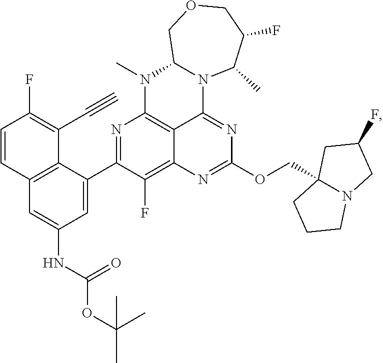 [SMILES: C#Cc1c(F)ccc2cc(NC(=O)OC(C)(C)C)cc(-c3nc4c5c(nc(OC[C@@]67CCCN6C[C@H](F)C7)nc5c3F)N3[C@@H](C)[C@@H](F)COC[C@H]3N4C)c12]

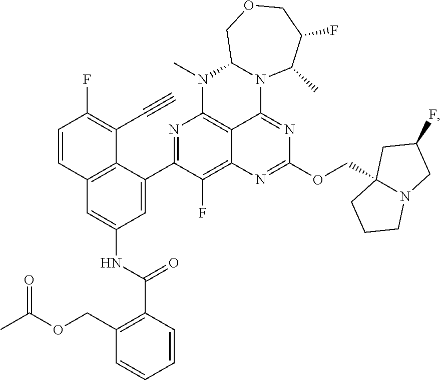 [SMILES: C#Cc1c(F)ccc2cc(NC(=O)c3ccccc3COC(C)=O)cc(-c3nc4c5c(nc(OC[C@@]67CCCN6C[C@H](F)C7)nc5c3F)N3[C@@H](C)[C@@H](F)COC[C@H]3N4C)c12]

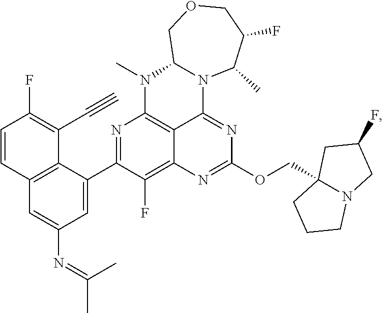 [SMILES: C#Cc1c(F)ccc2cc(N=C(C)C)cc(-c3nc4c5c(nc(OC[C@@]67CCCN6C[C@H](F)C7)nc5c3F)N3[C@@H](C)[C@@H](F)COC[C@H]3N4C)c12]

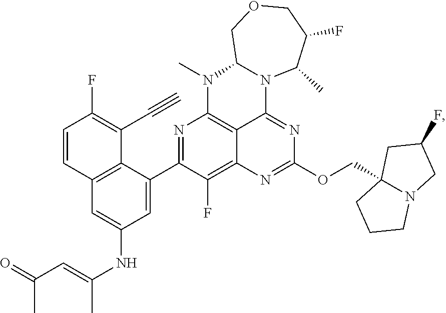 [SMILES: C#Cc1c(F)ccc2cc(N/C(C)=C/C(C)=O)cc(-c3nc4c5c(nc(OC[C@@]67CCCN6C[C@H](F)C7)nc5c3F)N3[C@@H](C)[C@@H](F)COC[C@H]3N4C)c12]

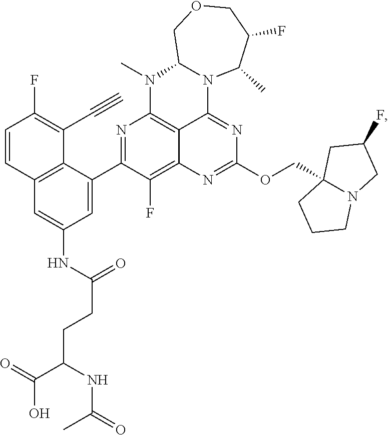 [SMILES: C#Cc1c(F)ccc2cc(NC(=O)CCC(NC(C)=O)C(=O)O)cc(-c3nc4c5c(nc(OC[C@@]67CCCN6C[C@H](F)C7)nc5c3F)N3[C@@H](C)[C@@H](F)COC[C@H]3N4C)c12]

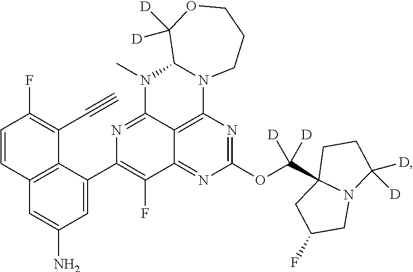

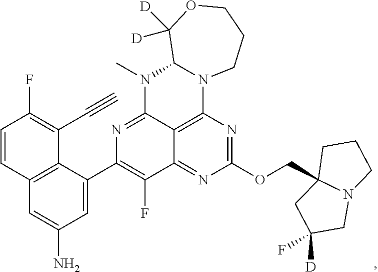 [SMILES: [2H]C1([2H])OCCCN2c3nc(OC[C@@]45CCCN4C[C@]([2H])(F)C5)nc4c(F)c(-c5cc(N)cc6ccc(F)c(C#C)c56)nc(c34)N(C)[C@@H]21]

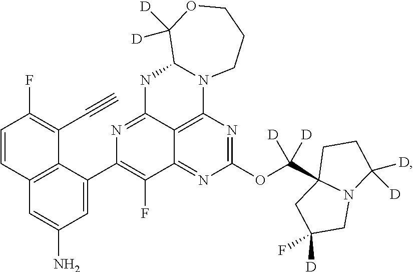

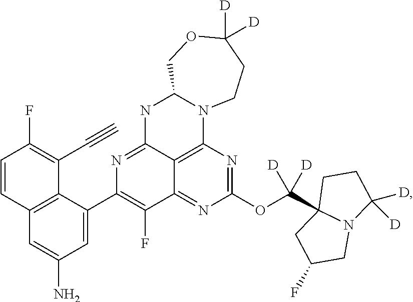

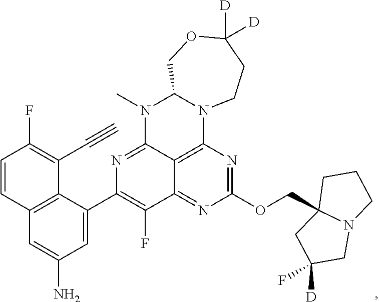 [SMILES: [2H]C1([2H])CCN2c3nc(OC[C@@]45CCCN4C[C@]([2H])(F)C5)nc4c(F)c(-c5cc(N)cc6ccc(F)c(C#C)c56)nc(c34)N(C)[C@@H]2CO1]

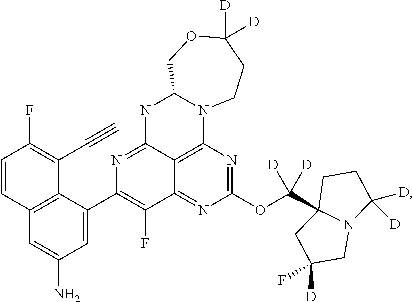

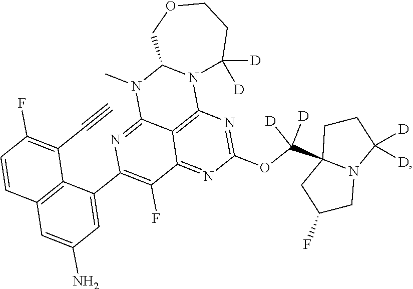

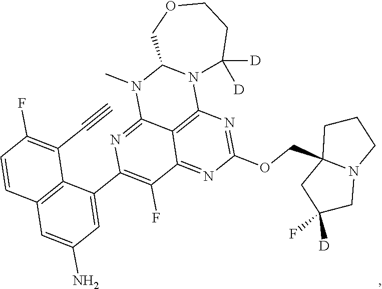 [SMILES: [2H]C1([2H])CCOC[C@H]2N(C)c3nc(-c4cc(N)cc5ccc(F)c(C#C)c45)c(F)c4nc(OC[C@@]56CCCN5C[C@]([2H])(F)C6)nc(c34)N21]

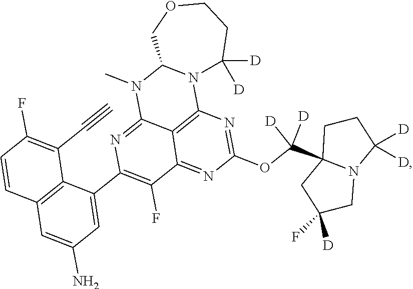

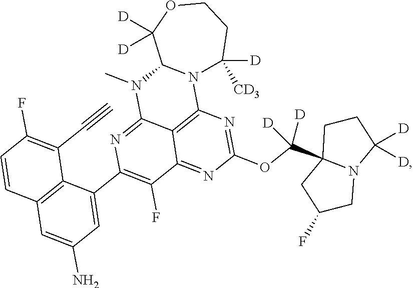

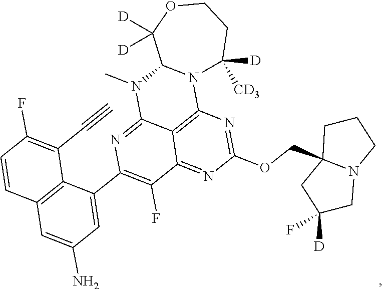 [SMILES: [2H]C1([2H])OCC[C@]([2H])(C([2H])([2H])[2H])N2c3nc(OC[C@@]45CCCN4C[C@]([2H])(F)C5)nc4c(F)c(-c5cc(N)cc6ccc(F)c(C#C)c56)nc(c34)N(C)[C@@H]21]

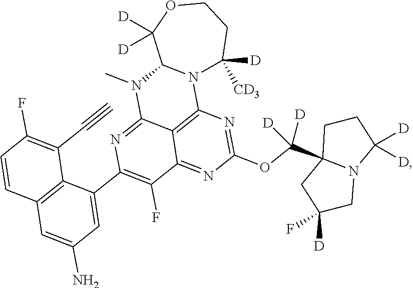

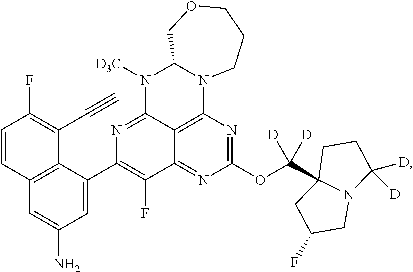

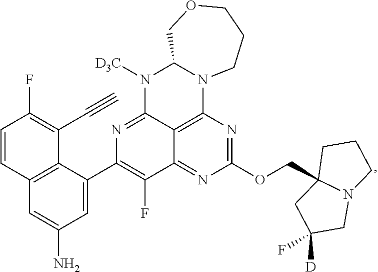 [SMILES: [2H]C([2H])([2H])N1c2nc(-c3cc(N)cc4ccc(F)c(C#C)c34)c(F)c3nc(OC[C@@]45CCCN4C[C@]([2H])(F)C5)nc(c23)N2CCCOC[C@H]21 ; ","]

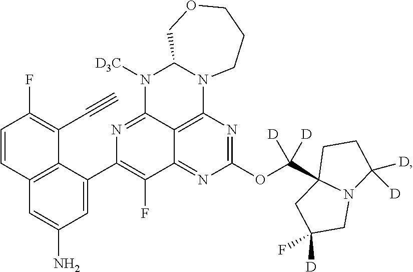

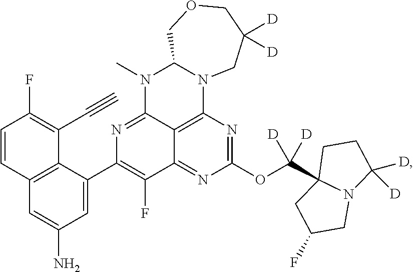

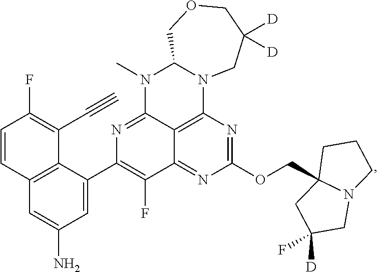 [SMILES: [2H]C1([2H])COC[C@H]2N(C)c3nc(-c4cc(N)cc5ccc(F)c(C#C)c45)c(F)c4nc(OC[C@@]56CCCN5C[C@]([2H])(F)C6)nc(c34)N2C1 ; ","]

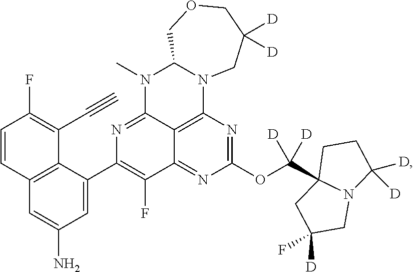

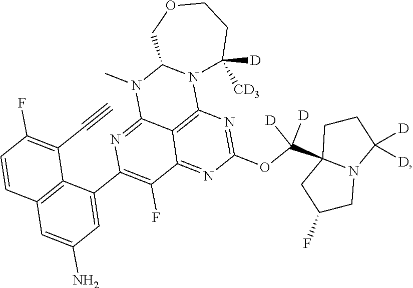

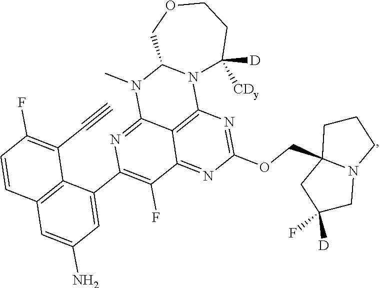

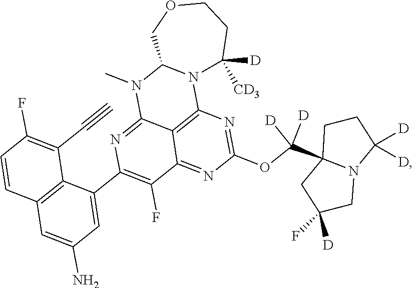

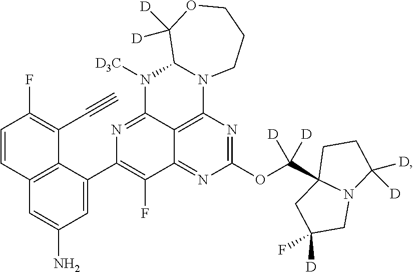

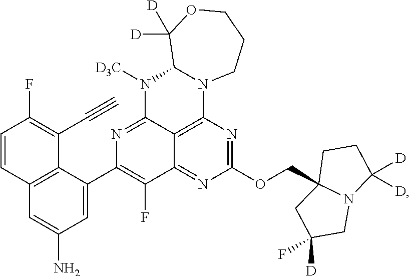

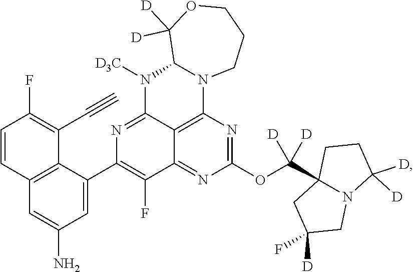

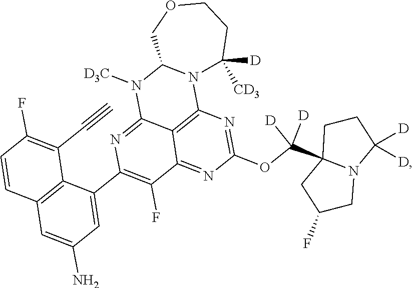

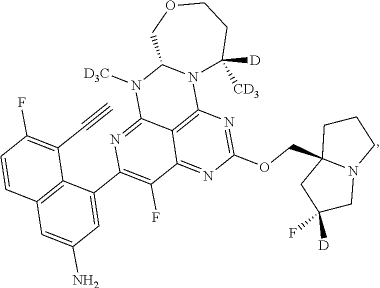 [SMILES: [2H]C([2H])([2H])N1c2nc(-c3cc(N)cc4ccc(F)c(C#C)c34)c(F)c3nc(OC[C@@]45CCCN4C[C@]([2H])(F)C5)nc(c23)N2[C@H]1COCC[C@]2([2H])C([2H])([2H])[2H] ; ","]

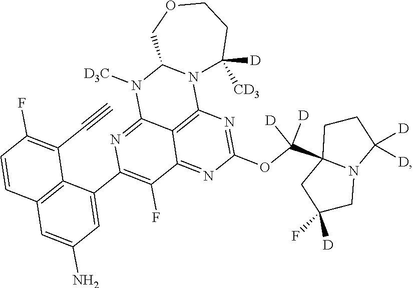

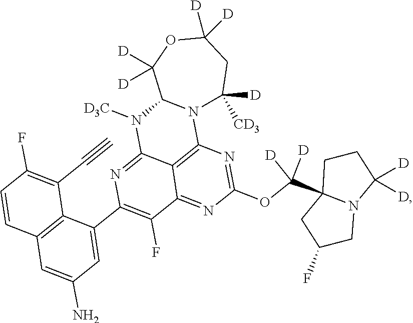

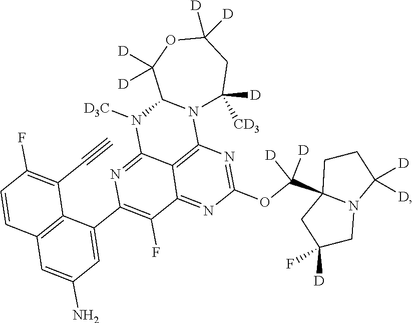

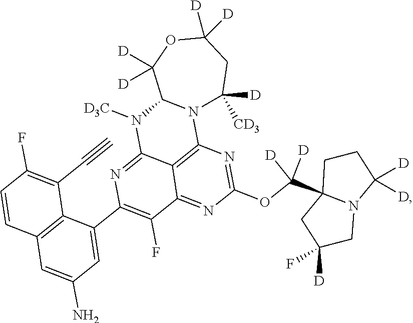

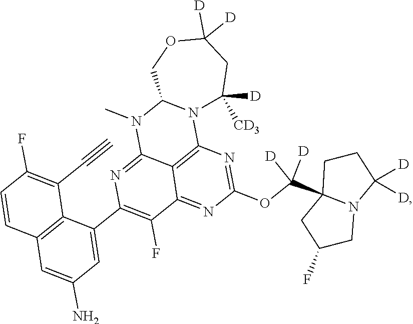

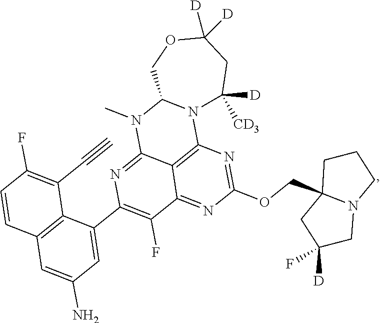 [SMILES: [2H]C1([2H])C[C@]([2H])(C([2H])([2H])[2H])N2c3nc(OC[C@@]45CCCN4C[C@]([2H])(F)C5)nc4c(F)c(-c5cc(N)cc6ccc(F)c(C#C)c56)nc(c34)N(C)[C@@H]2CO1 ; ","]

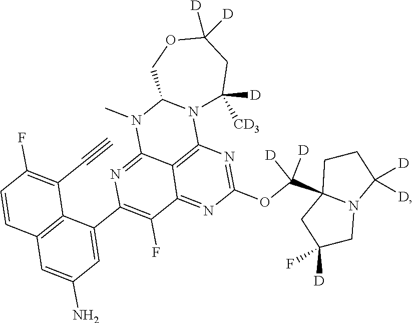

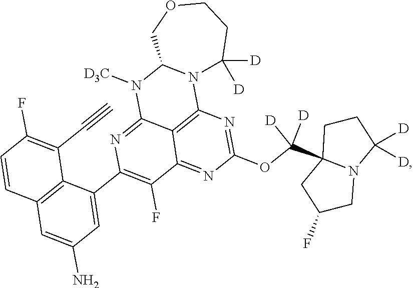

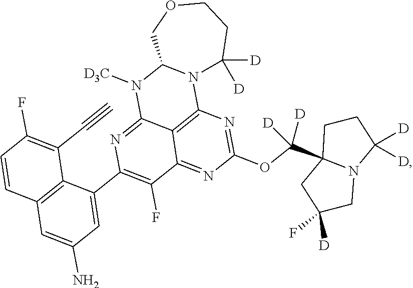

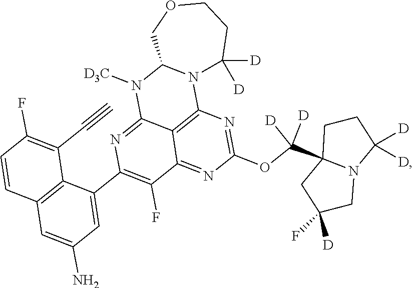

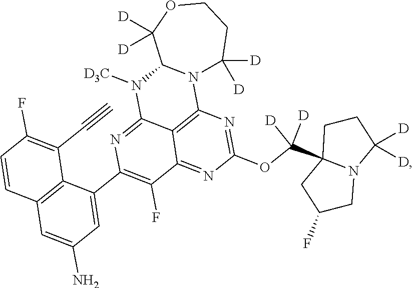

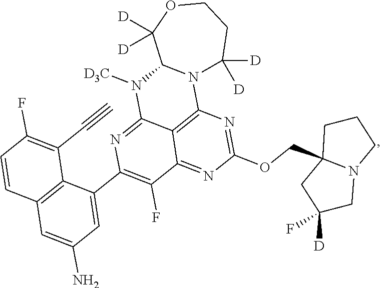 [SMILES: [2H]C1([2H])OCCC([2H])([2H])N2c3nc(OC[C@@]45CCCN4C[C@]([2H])(F)C5)nc4c(F)c(-c5cc(N)cc6ccc(F)c(C#C)c56)nc(c34)N(C([2H])([2H])[2H])[C@@H]21 ; ","]

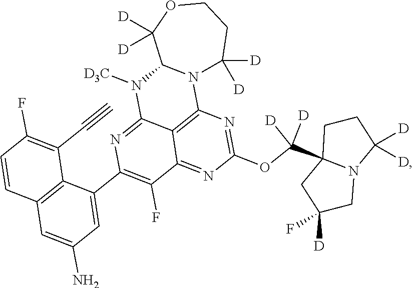

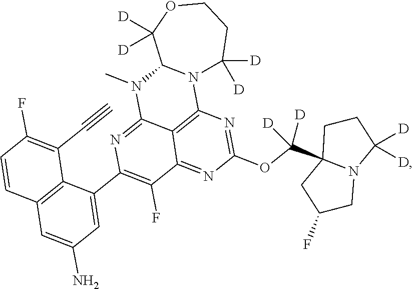

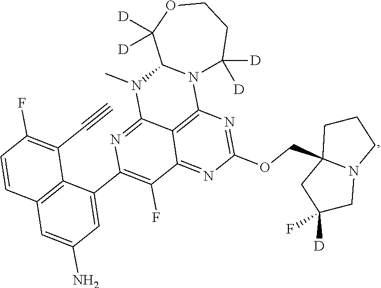 [SMILES: [2H]C1([2H])OCCC([2H])([2H])N2c3nc(OC[C@@]45CCCN4C[C@]([2H])(F)C5)nc4c(F)c(-c5cc(N)cc6ccc(F)c(C#C)c56)nc(c34)N(C)[C@@H]21 ; ","]

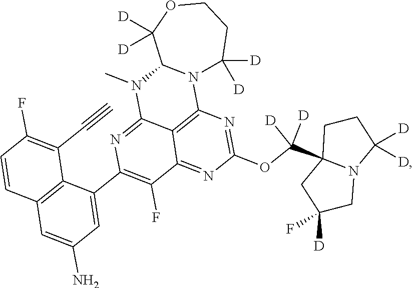

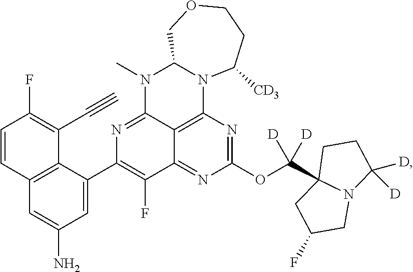

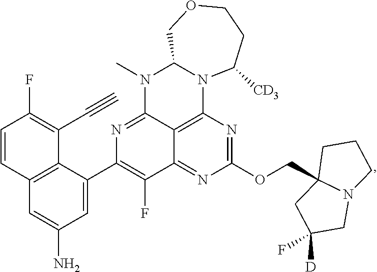 [SMILES: [2H]C([2H])([2H])[C@H]1CCOC[C@H]2N(C)c3nc(-c4cc(N)cc5ccc(F)c(C#C)c45)c(F)c4nc(OC[C@@]56CCCN5C[C@]([2H])(F)C6)nc(c34)N21 ; ","]

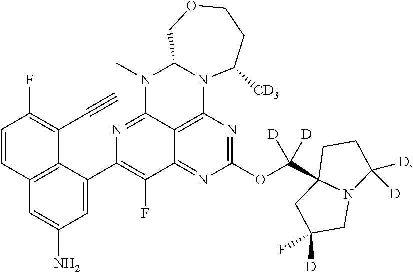

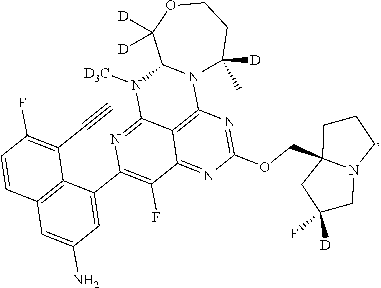 [SMILES: [2H]C1([2H])OCC[C@]([2H])(C)N2c3nc(OC[C@@]45CCCN4C[C@]([2H])(F)C5)nc4c(F)c(-c5cc(N)cc6ccc(F)c(C#C)c56)nc(c34)N(C([2H])([2H])[2H])[C@@H]21 ; ","]

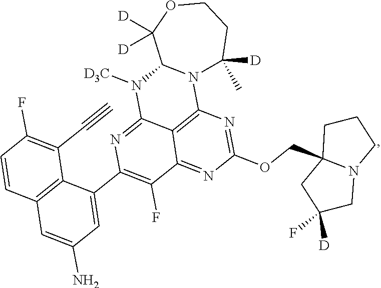 [SMILES: [2H]C1([2H])OCC[C@]([2H])(C)N2c3nc(OC[C@@]45CCCN4C[C@]([2H])(F)C5)nc4c(F)c(-c5cc(N)cc6ccc(F)c(C#C)c56)nc(c34)N(C([2H])([2H])[2H])[C@@H]21 ; ","]

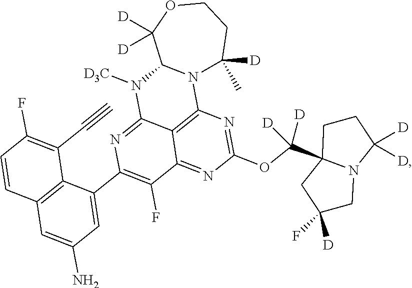

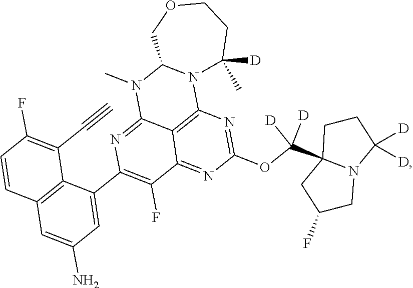

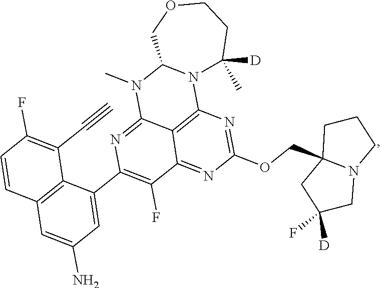 [SMILES: [2H][C@]1(F)CN2CCC[C@@]2(COc2nc3c4c(nc(-c5cc(N)cc6ccc(F)c(C#C)c56)c(F)c4n2)N(C)[C@@H]2COCC[C@]([2H])(C)N32)C1 ; ","]

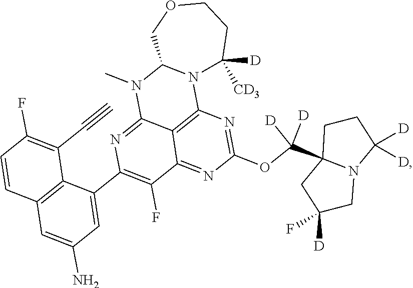

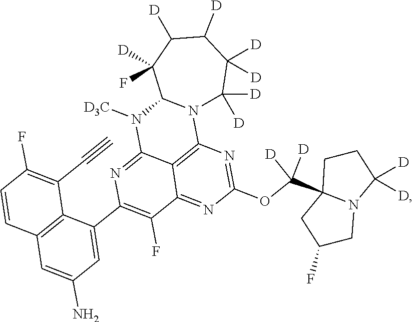

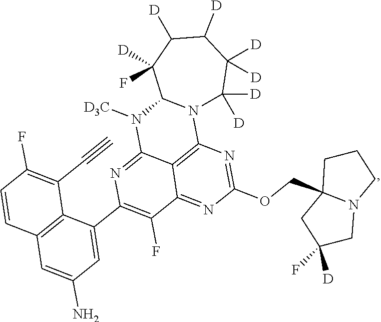 [SMILES: [2H]C1C([2H])[C@]([2H])(F)[C@H]2N(C([2H])([2H])[2H])c3nc(-c4cc(N)cc5ccc(F)c(C#C)c45)c(F)c4nc(OC[C@@]56CCCN5C[C@]([2H])(F)C6)nc(c34)N2C([2H])([2H])C1([2H])[2H] ; ","]

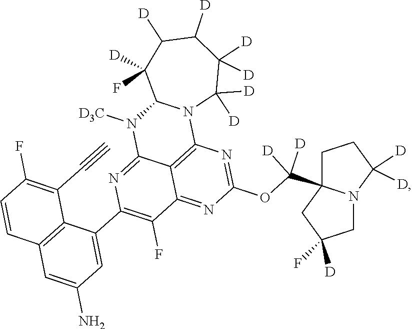

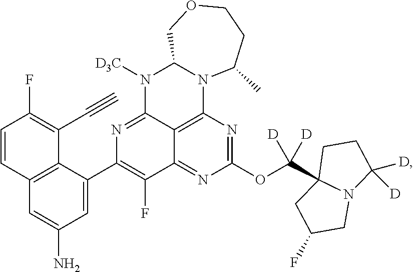

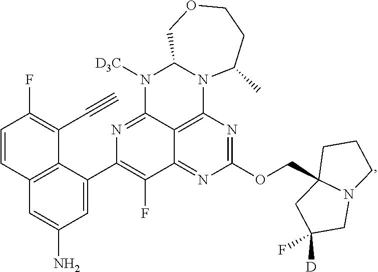 [SMILES: [2H]C([2H])([2H])N1c2nc(-c3cc(N)cc4ccc(F)c(C#C)c34)c(F)c3nc(OC[C@@]45CCCN4C[C@]([2H])(F)C5)nc(c23)N2[C@H]1COCC[C@@H]2C ; ","]

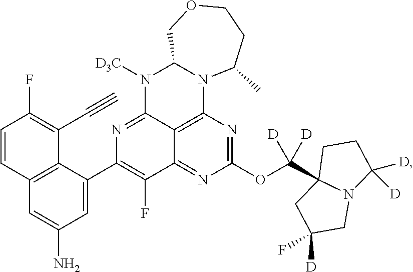

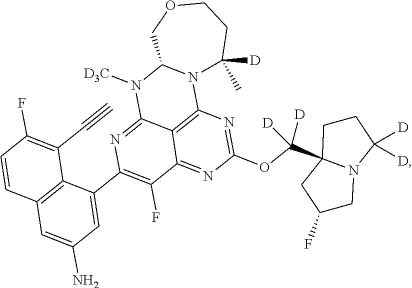

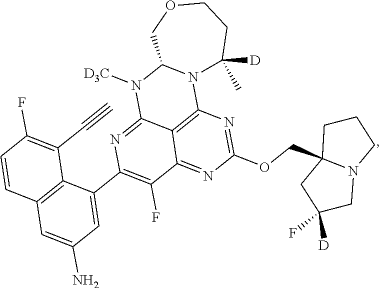 [SMILES: [2H]C([2H])([2H])N1c2nc(-c3cc(N)cc4ccc(F)c(C#C)c34)c(F)c3nc(OC[C@@]45CCCN4C[C@]([2H])(F)C5)nc(c23)N2[C@H]1COCC[C@]2([2H])C ; ","]

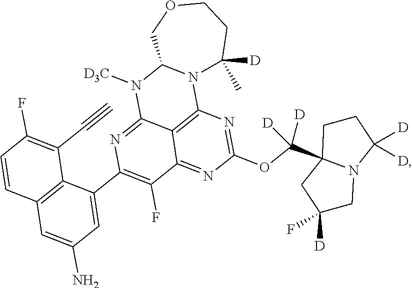

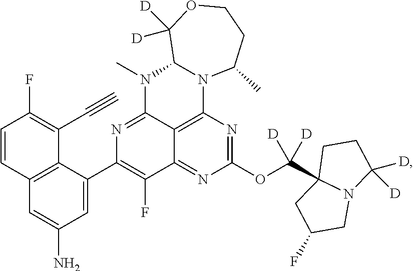

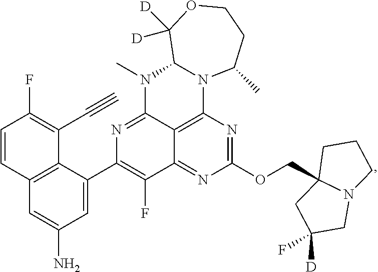 [SMILES: [2H]C1([2H])OCC[C@H](C)N2c3nc(OC[C@@]45CCCN4C[C@]([2H])(F)C5)nc4c(F)c(-c5cc(N)cc6ccc(F)c(C#C)c56)nc(c34)N(C)[C@@H]21 ; ","]

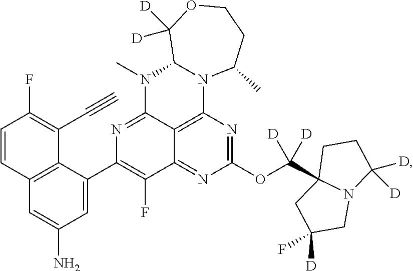

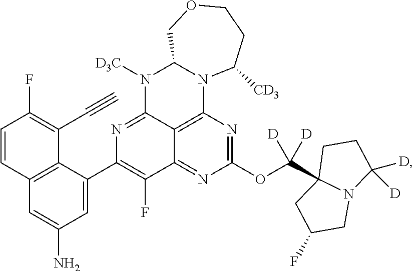

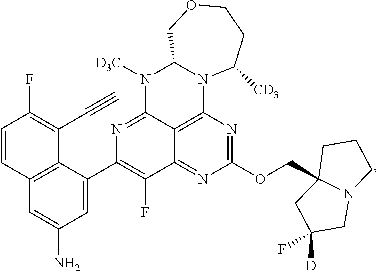 [SMILES: [2H]C([2H])([2H])[C@H]1CCOC[C@@H]2N1c1nc(OC[C@@]34CCCN3C[C@]([2H])(F)C4)nc3c(F)c(-c4cc(N)cc5ccc(F)c(C#C)c45)nc(c13)N2C([2H])([2H])[2H] ; ","]

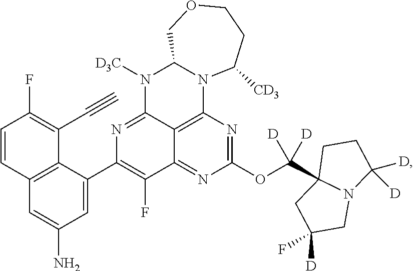

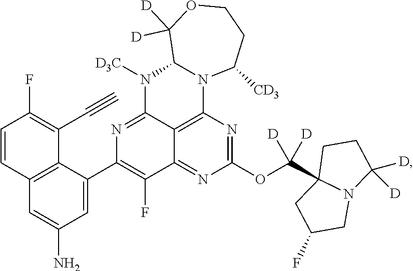

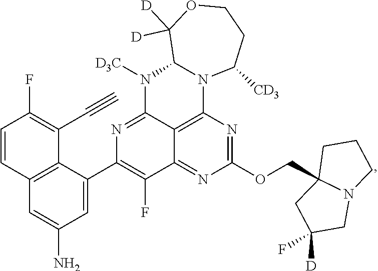 [SMILES: [2H]C([2H])([2H])[C@H]1CCOC([2H])([2H])[C@@H]2N1c1nc(OC[C@@]34CCCN3C[C@]([2H])(F)C4)nc3c(F)c(-c4cc(N)cc5ccc(F)c(C#C)c45)nc(c13)N2C([2H])([2H])[2H] ; ","]

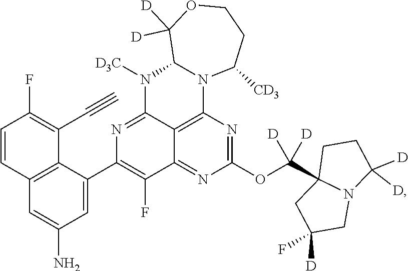

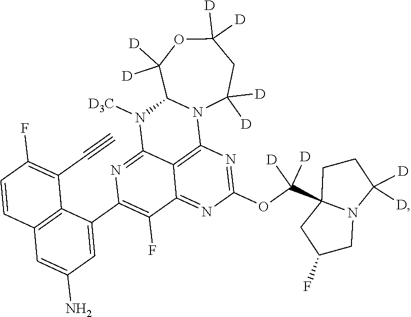

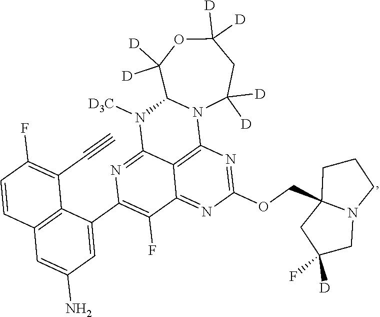 [SMILES: [2H]C1([2H])CC([2H])([2H])N2c3nc(OC[C@@]45CCCN4C[C@]([2H])(F)C5)nc4c(F)c(-c5cc(N)cc6ccc(F)c(C#C)c56)nc(c34)N(C([2H])([2H])[2H])[C@@H]2C([2H])([2H])O1 ; ","]

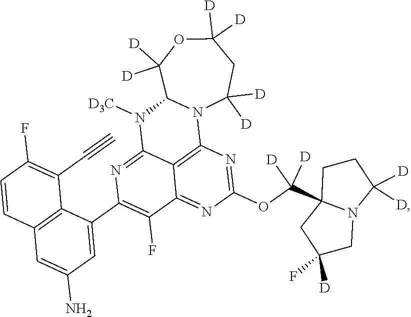

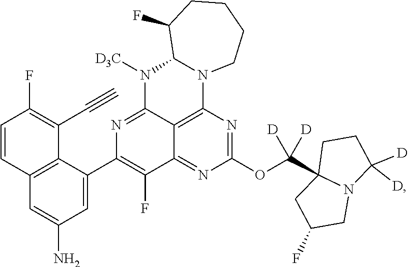

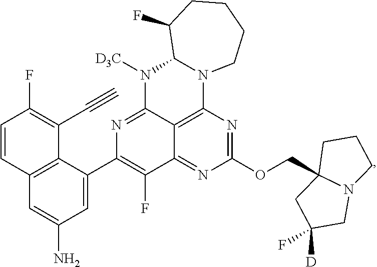 [SMILES: [2H]C([2H])([2H])N1c2nc(-c3cc(N)cc4ccc(F)c(C#C)c34)c(F)c3nc(OC[C@@]45CCCN4C[C@]([2H])(F)C5)nc(c23)N2CCCC[C@H](F)[C@H]21 ; ","]

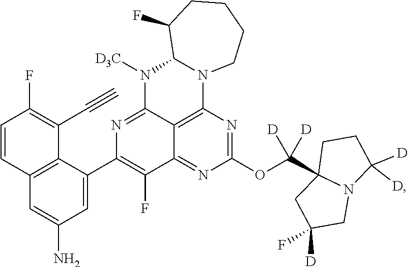

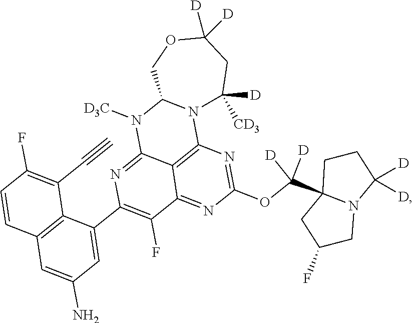

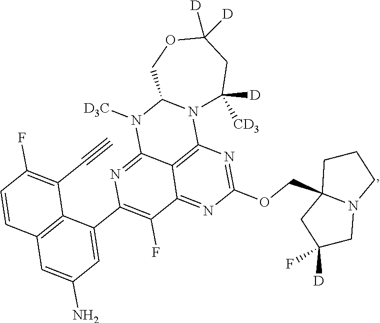 [SMILES: [2H]C1([2H])C[C@]([2H])(C([2H])([2H])[2H])N2c3nc(OC[C@@]45CCCN4C[C@]([2H])(F)C5)nc4c(F)c(-c5cc(N)cc6ccc(F)c(C#C)c56)nc(c34)N(C([2H])([2H])[2H])[C@@H]2CO1 ; ","]

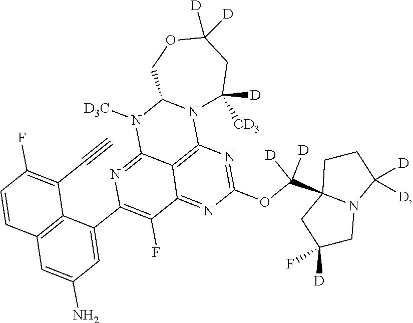

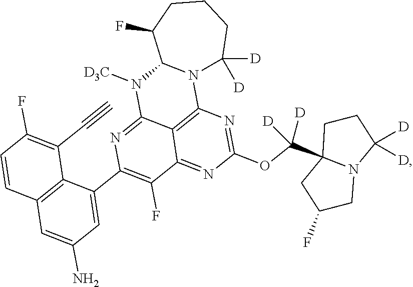

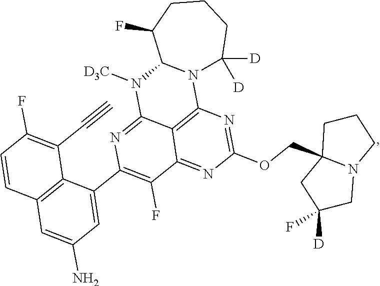 [SMILES: [2H]C([2H])([2H])N1c2nc(-c3cc(N)cc4ccc(F)c(C#C)c34)c(F)c3nc(OC[C@@]45CCCN4C[C@]([2H])(F)C5)nc(c23)N2[C@H]1[C@@H](F)CCCC2([2H])[2H] ; ","]

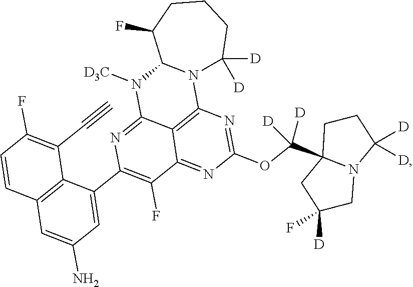

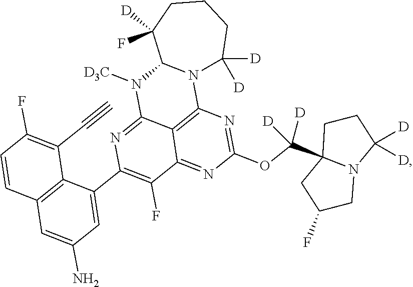

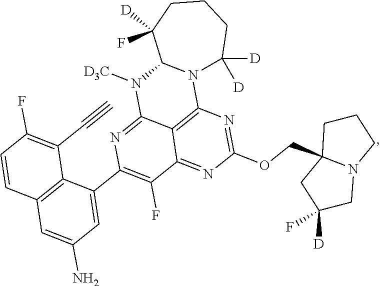 [SMILES: [2H]C([2H])([2H])N1c2nc(-c3cc(N)cc4ccc(F)c(C#C)c34)c(F)c3nc(OC[C@@]45CCCN4C[C@]([2H])(F)C5)nc(c23)N2[C@H]1[C@@]([2H])(F)CCCC2([2H])[2H] ; ","]

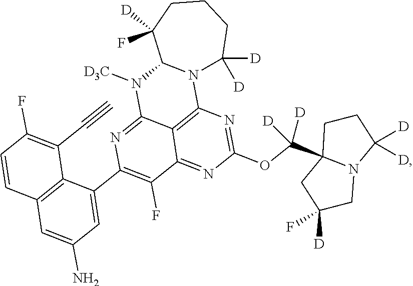

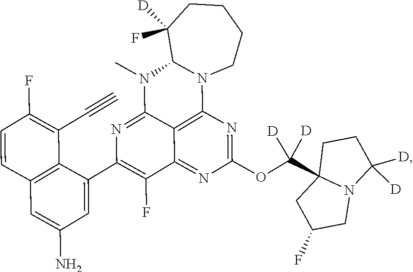

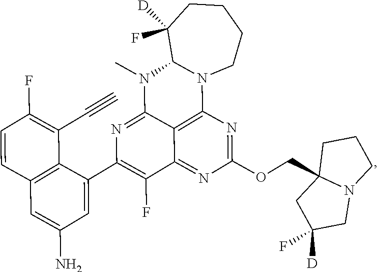 [SMILES: [2H][C@]1(F)CN2CCC[C@@]2(COc2nc3c4c(nc(-c5cc(N)cc6ccc(F)c(C#C)c56)c(F)c4n2)N(C)[C@H]2N3CCCC[C@]2([2H])F)C1 ; ","]

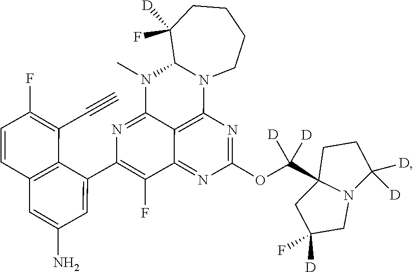

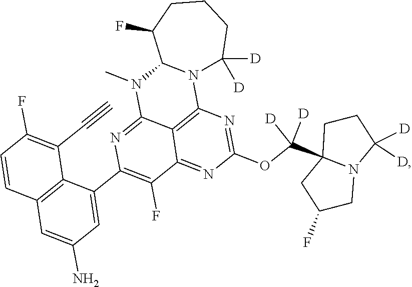

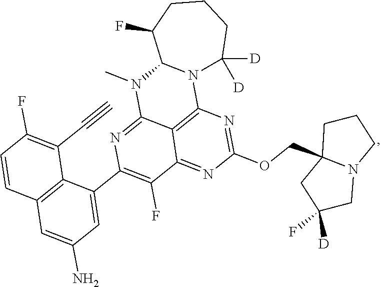 [SMILES: [2H]C1([2H])CCC[C@H](F)[C@H]2N(C)c3nc(-c4cc(N)cc5ccc(F)c(C#C)c45)c(F)c4nc(OC[C@@]56CCCN5C[C@]([2H])(F)C6)nc(c34)N21 ; ","]

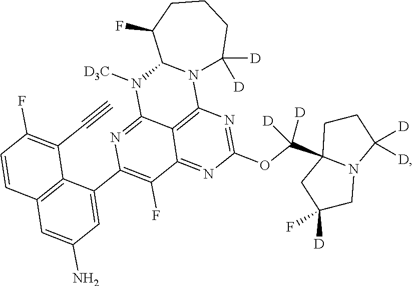

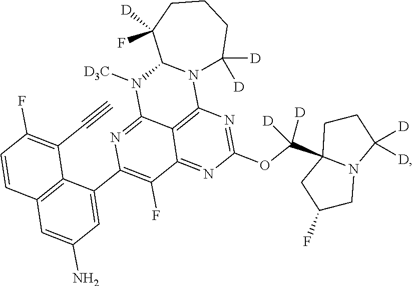

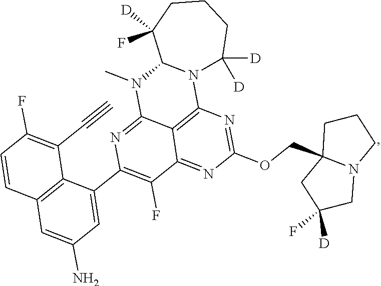 [SMILES: [2H]C1([2H])CCC[C@]([2H])(F)[C@H]2N(C)c3nc(-c4cc(N)cc5ccc(F)c(C#C)c45)c(F)c4nc(OC[C@@]56CCCN5C[C@]([2H])(F)C6)nc(c34)N21 ; ","]

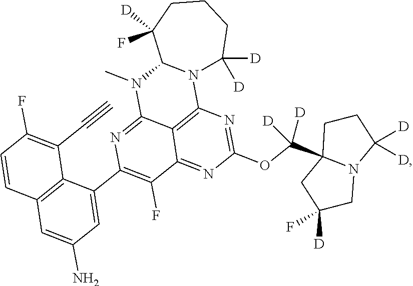

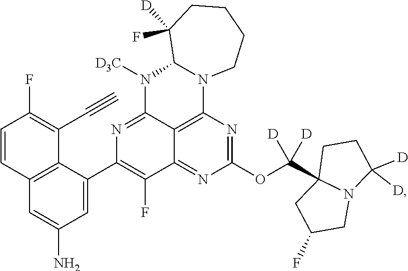

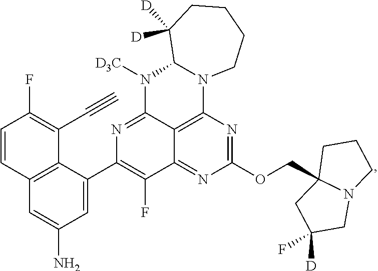

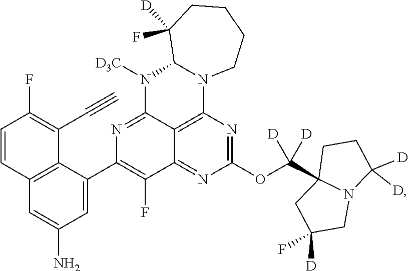

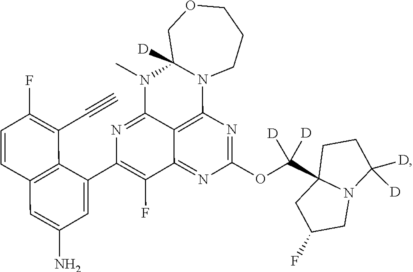

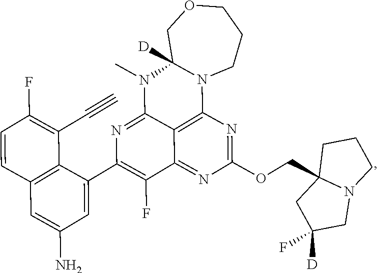 [SMILES: [2H][C@]1(F)CN2CCC[C@@]2(COc2nc3c4c(nc(-c5cc(N)cc6ccc(F)c(C#C)c56)c(F)c4n2)N(C)[C@]2([2H])COCCCN32)C1 ; ","]

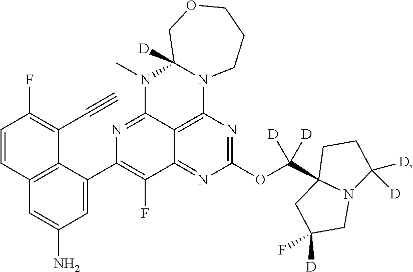

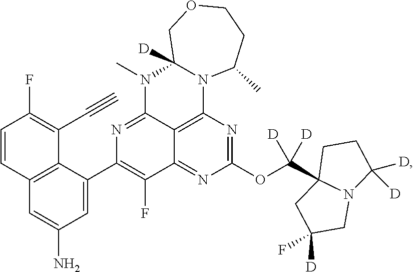

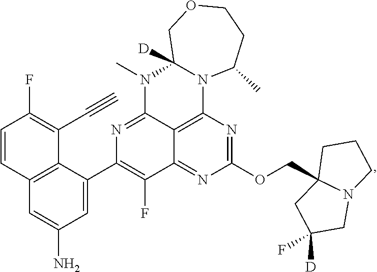 [SMILES: [2H][C@]1(F)CN2CCC[C@@]2(COc2nc3c4c(nc(-c5cc(N)cc6ccc(F)c(C#C)c56)c(F)c4n2)N(C)[C@]2([2H])COCC[C@H](C)N32)C1 ; ","]

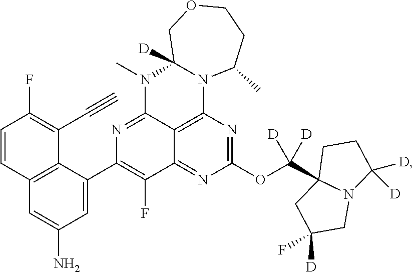

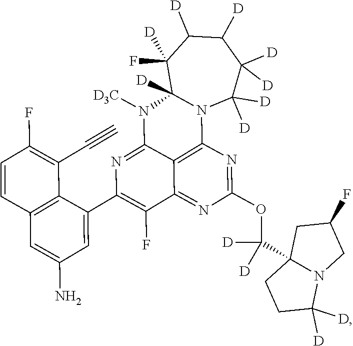

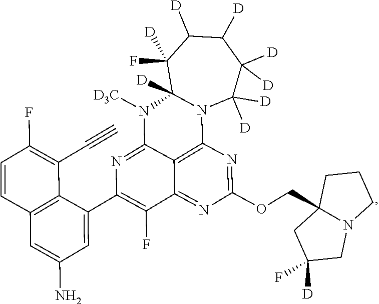 [SMILES: [2H]C1C([2H])[C@]([2H])(F)[C@@]2([2H])N(C([2H])([2H])[2H])c3nc(-c4cc(N)cc5ccc(F)c(C#C)c45)c(F)c4nc(OC[C@@]56CCCN5C[C@]([2H])(F)C6)nc(c34)N2C([2H])([2H])C1([2H])[2H] ; ","]

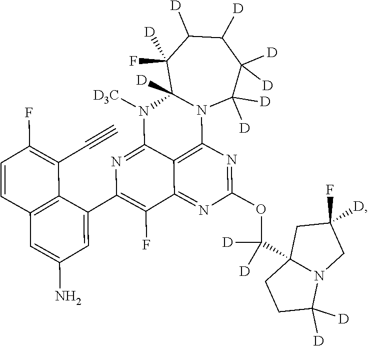

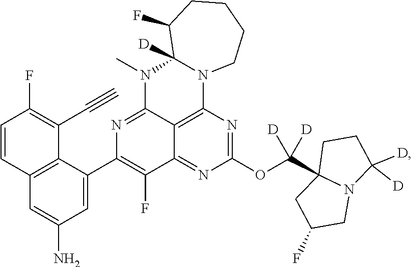

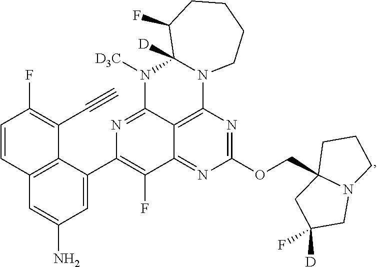 [SMILES: [2H]C([2H])([2H])N1c2nc(-c3cc(N)cc4ccc(F)c(C#C)c34)c(F)c3nc(OC[C@@]45CCCN4C[C@]([2H])(F)C5)nc(c23)N2CCCC[C@H](F)[C@]21[2H] ; ","]

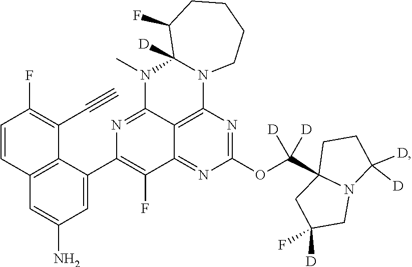

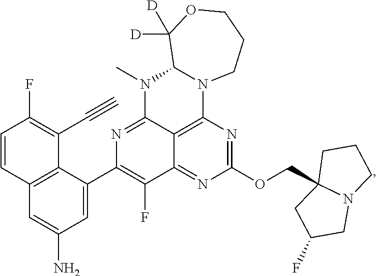 [SMILES: [2H]C1([2H])OCCCN2c3nc(OC[C@@]45CCCN4C[C@H](F)C5)nc4c(F)c(-c5cc(N)cc6ccc(F)c(C#C)c56)nc(c34)N(C)[C@@H]21 ; ","]

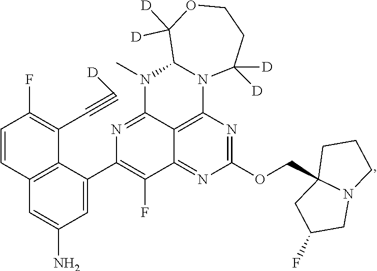 [SMILES: [2H]C#Cc1c(F)ccc2cc(N)cc(-c3nc4c5c(nc(OC[C@@]67CCCN6C[C@H](F)C7)nc5c3F)N3[C@H](N4C)C([2H])([2H])OCCC3([2H])[2H])c12 ; ","]

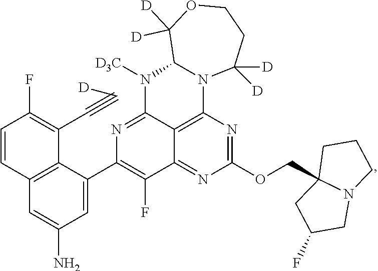 [SMILES: [2H]C#Cc1c(F)ccc2cc(N)cc(-c3nc4c5c(nc(OC[C@@]67CCCN6C[C@H](F)C7)nc5c3F)N3[C@H](N4C([2H])([2H])[2H])C([2H])([2H])OCCC3([2H])[2H])c12 ; ","]

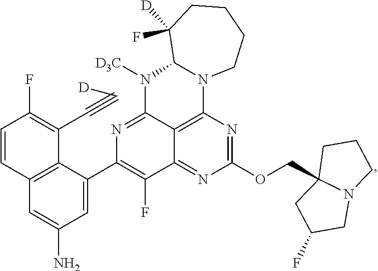 [SMILES: [2H]C#Cc1c(F)ccc2cc(N)cc(-c3nc4c5c(nc(OC[C@@]67CCCN6C[C@H](F)C7)nc5c3F)N3CCCC[C@]([2H])(F)[C@H]3N4C([2H])([2H])[2H])c12 ; ","]

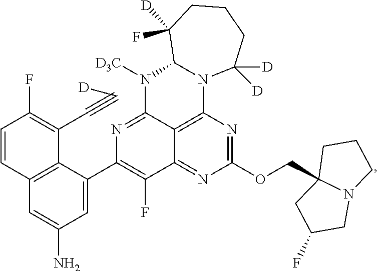 [SMILES: [2H]C#Cc1c(F)ccc2cc(N)cc(-c3nc4c5c(nc(OC[C@@]67CCCN6C[C@H](F)C7)nc5c3F)N3[C@H](N4C([2H])([2H])[2H])[C@@]([2H])(F)CCCC3([2H])[2H])c12 ; ","]

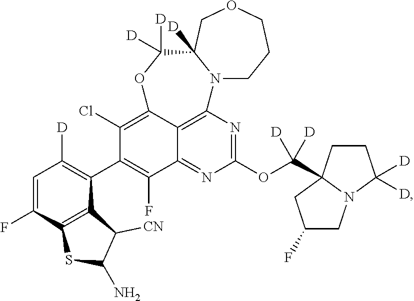

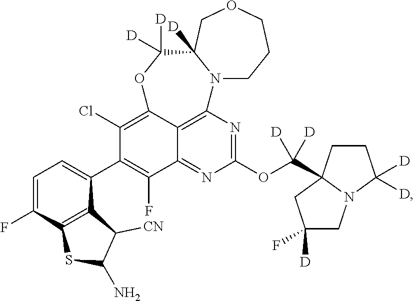

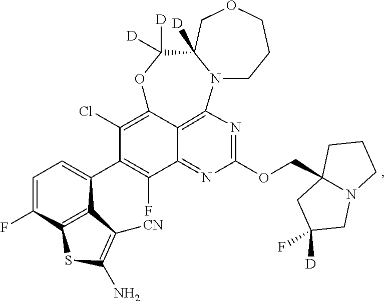

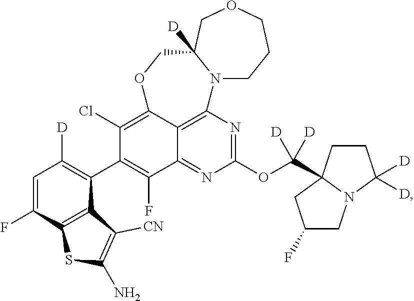

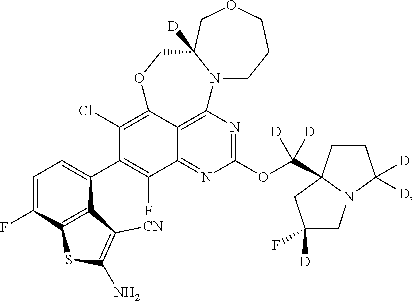

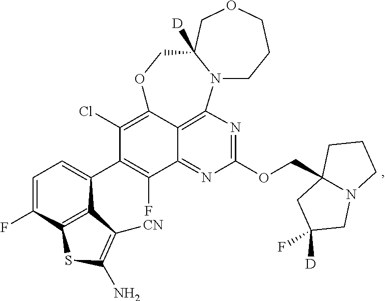

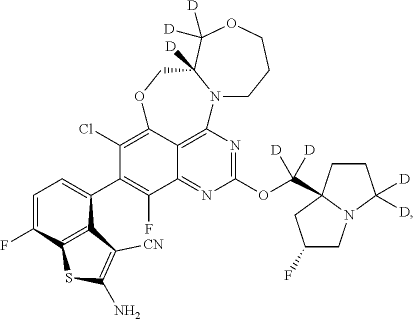

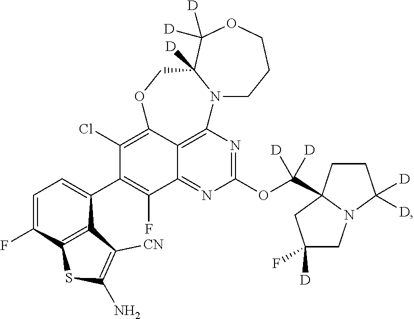

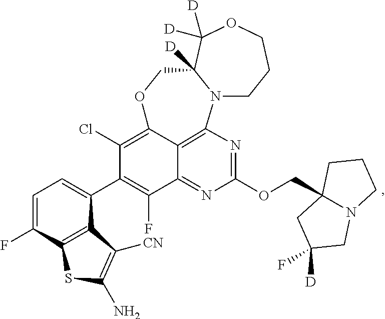

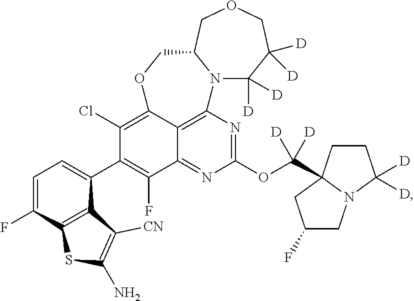

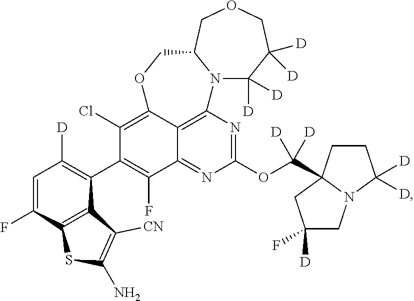

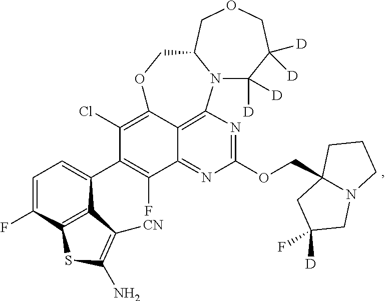

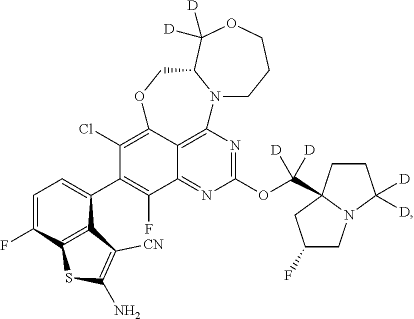

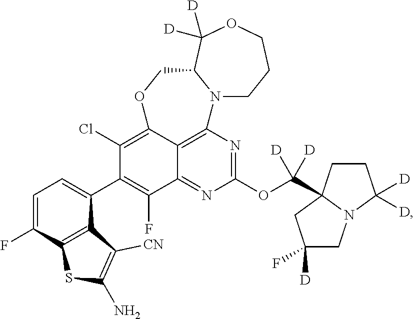

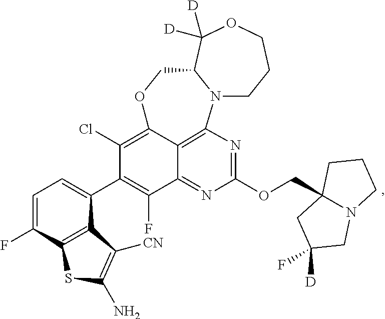

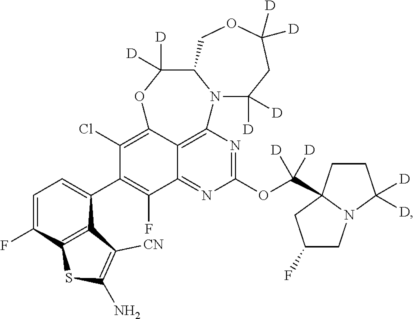

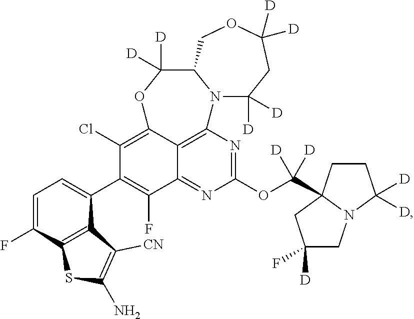

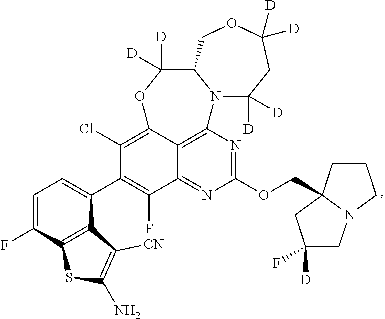

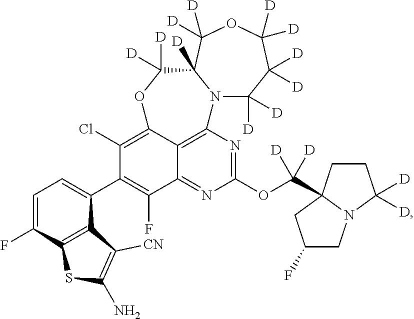

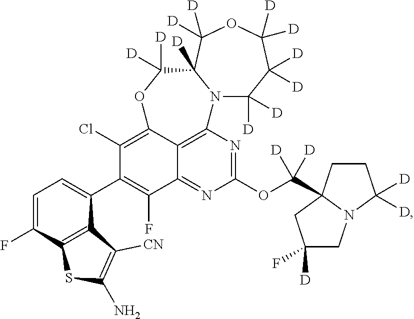

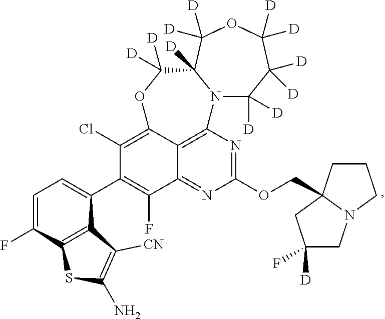

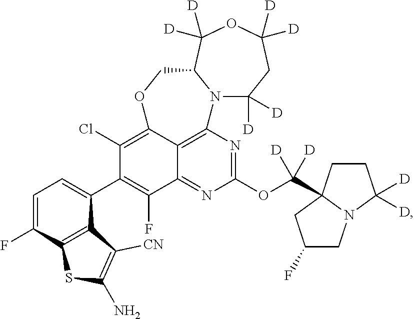

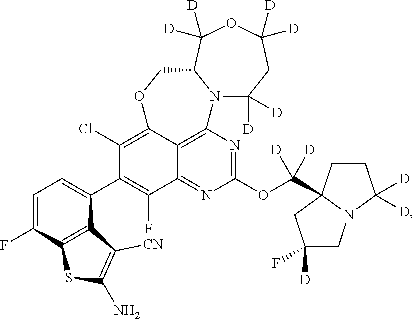

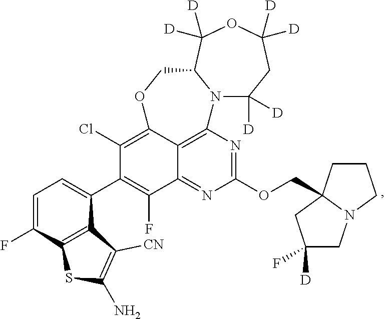

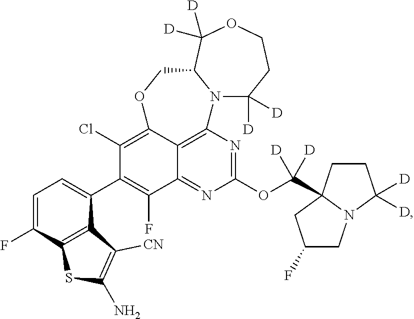

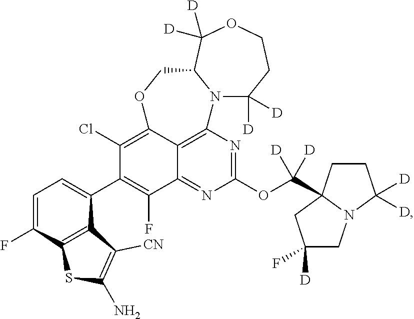

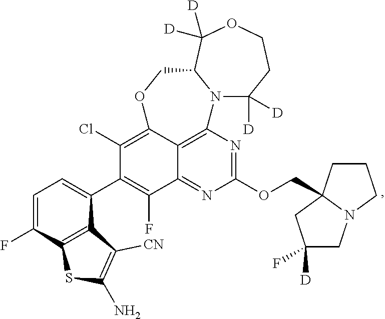

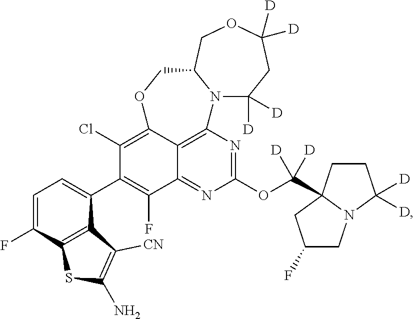

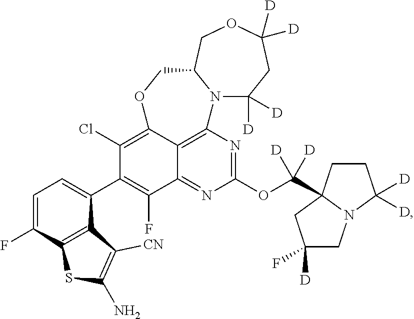

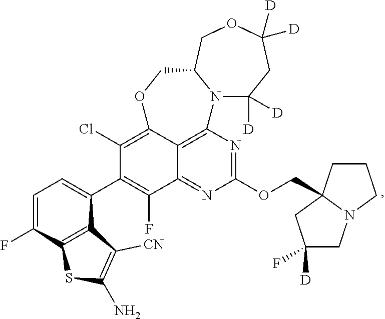

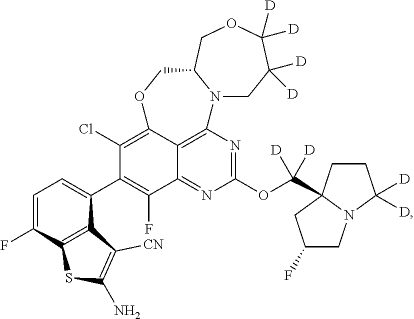

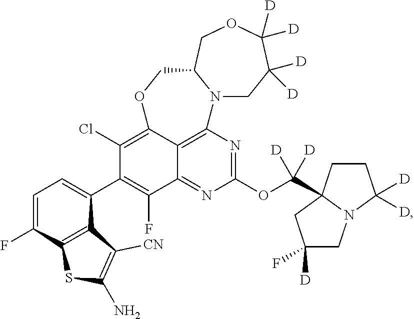

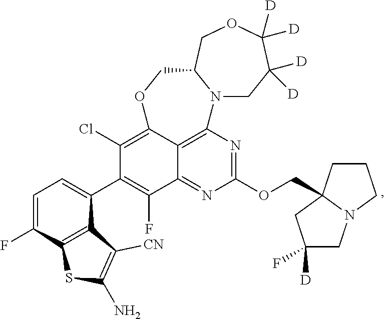

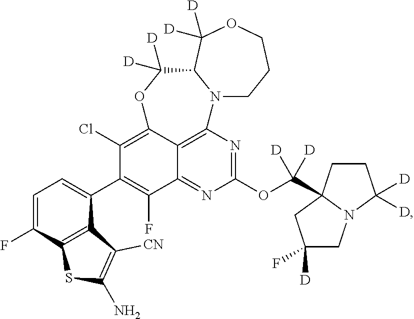

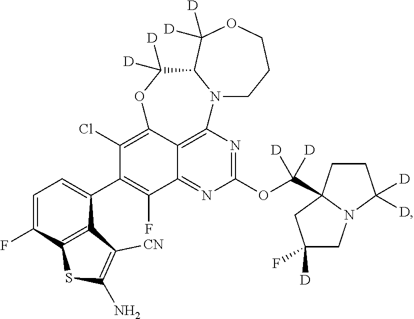

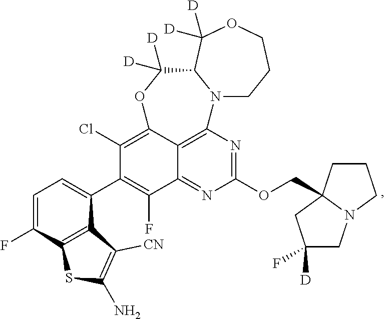

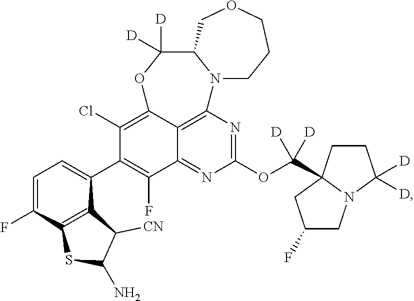

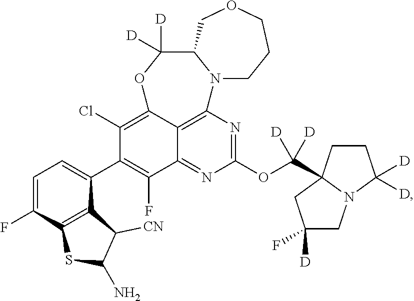

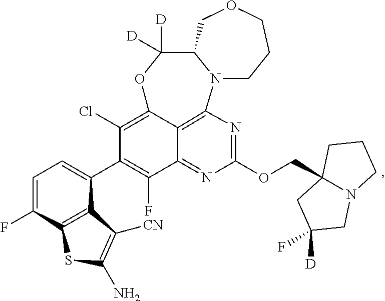

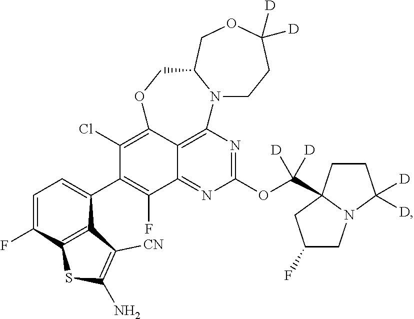

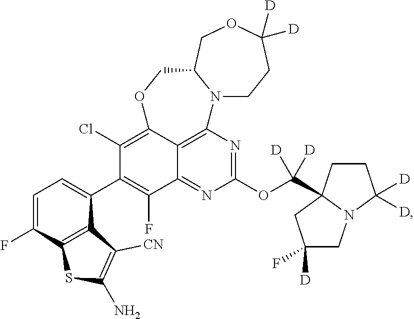

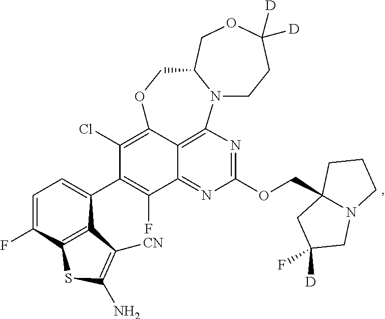

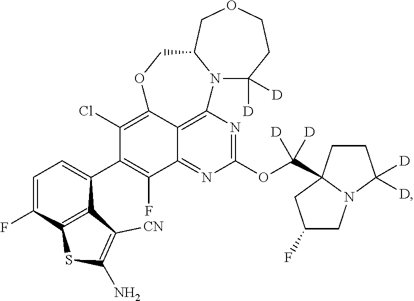

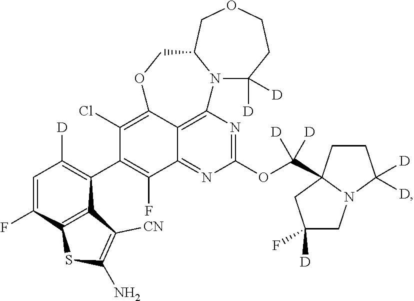

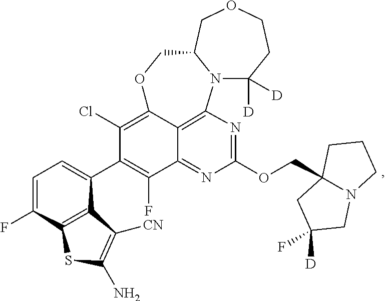

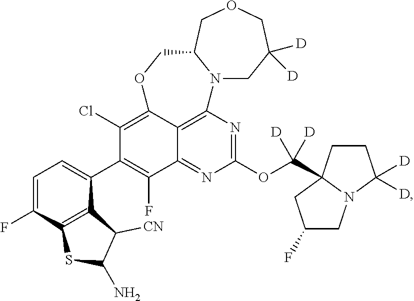

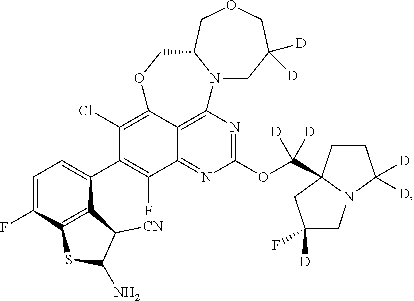

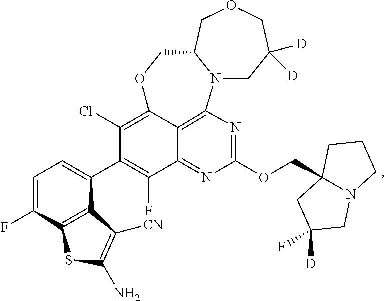

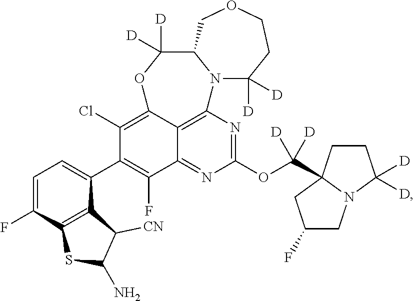

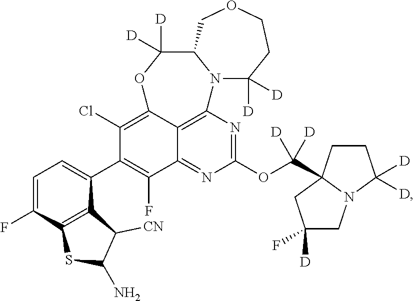

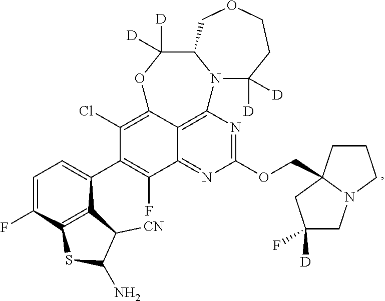

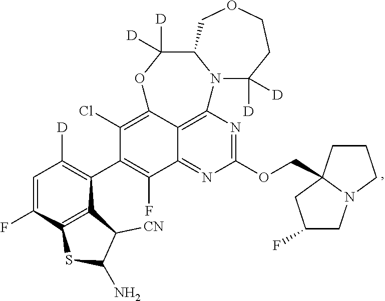

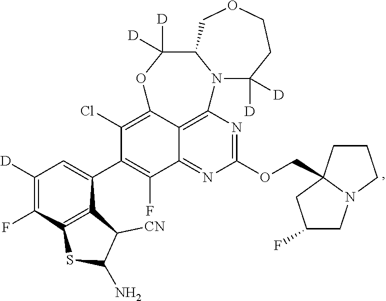

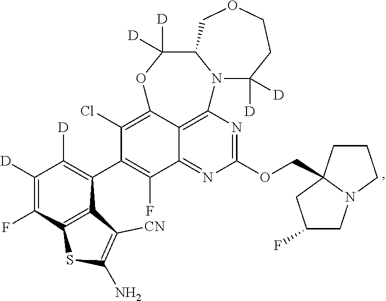

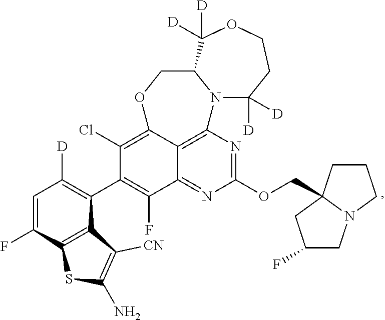

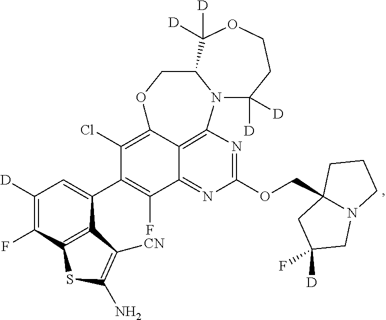

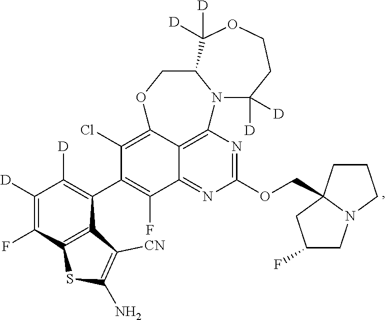

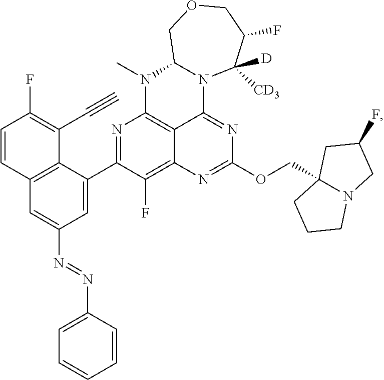 [SMILES: [2H]C([2H])([2H])[C@@]1([2H])[C@@H](F)COC[C@H]2N(C)c3nc(-c4cc(/N=N/c5ccccc5)cc5ccc(F)c(C#C)c45)c(F)c4nc(OC[C@@]56CCCN5C[C@H](F)C6)nc(c34)N21]

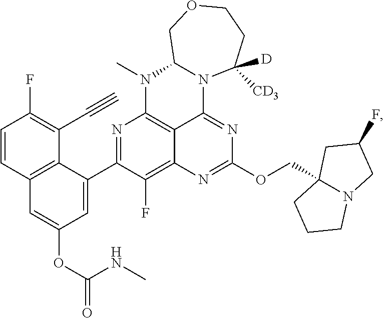 [SMILES: [2H]C([2H])([2H])[C@@]1([2H])CCOC[C@H]2N(C)c3nc(-c4cc(OC(=O)NC)cc5ccc(F)c(C#C)c45)c(F)c4nc(OC[C@@]56CCCN5C[C@H](F)C6)nc(c34)N21]

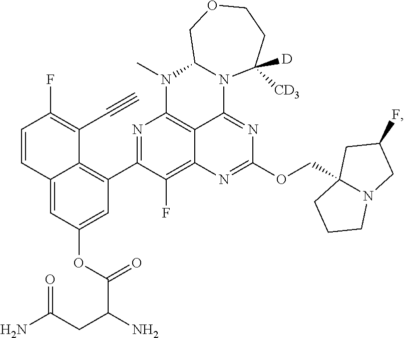 [SMILES: [2H]C([2H])([2H])[C@@]1([2H])CCOC[C@H]2N(C)c3nc(-c4cc(OC(=O)C(N)CC(N)=O)cc5ccc(F)c(C#C)c45)c(F)c4nc(OC[C@@]56CCCN5C[C@H](F)C6)nc(c34)N21]

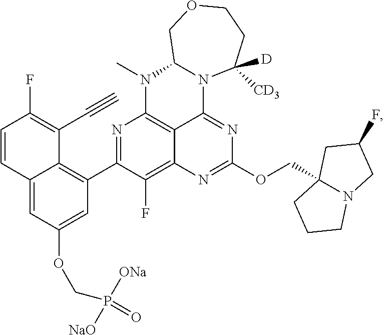

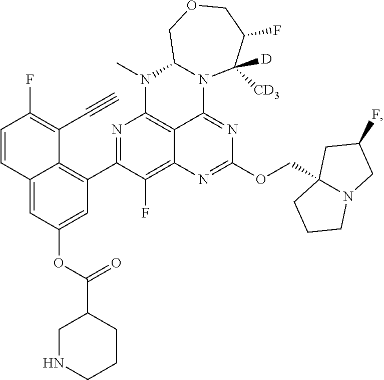 [SMILES: [2H]C([2H])([2H])[C@@]1([2H])[C@@H](F)COC[C@H]2N(C)c3nc(-c4cc(OC(=O)C5CCCNC5)cc5ccc(F)c(C#C)c45)c(F)c4nc(OC[C@@]56CCCN5C[C@H](F)C6)nc(c34)N21]

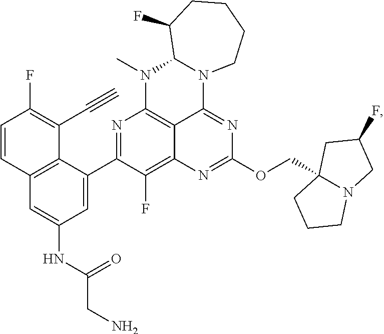 [SMILES: C#Cc1c(F)ccc2cc(NC(=O)CN)cc(-c3nc4c5c(nc(OC[C@@]67CCCN6C[C@H](F)C7)nc5c3F)N3CCCC[C@H](F)[C@H]3N4C)c12]

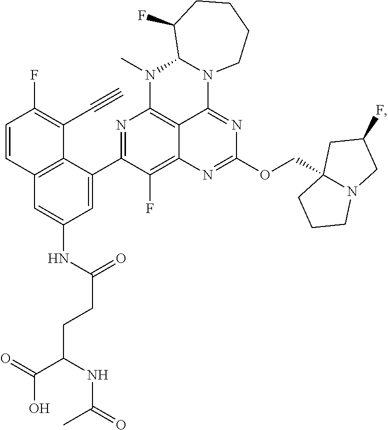 [SMILES: C#Cc1c(F)ccc2cc(NC(=O)CCC(NC(C)=O)C(=O)O)cc(-c3nc4c5c(nc(OC[C@@]67CCCN6C[C@H](F)C7)nc5c3F)N3CCCC[C@H](F)[C@H]3N4C)c12]

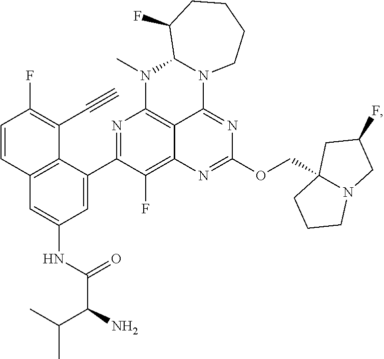 [SMILES: C#Cc1c(F)ccc2cc(NC(=O)[C@@H](N)C(C)C)cc(-c3nc4c5c(nc(OC[C@@]67CCCN6C[C@H](F)C7)nc5c3F)N3CCCC[C@H](F)[C@H]3N4C)c12]

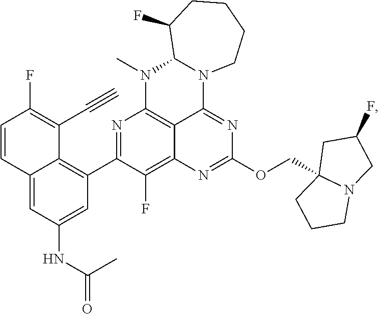 [SMILES: C#Cc1c(F)ccc2cc(NC(C)=O)cc(-c3nc4c5c(nc(OC[C@@]67CCCN6C[C@H](F)C7)nc5c3F)N3CCCC[C@H](F)[C@H]3N4C)c12]

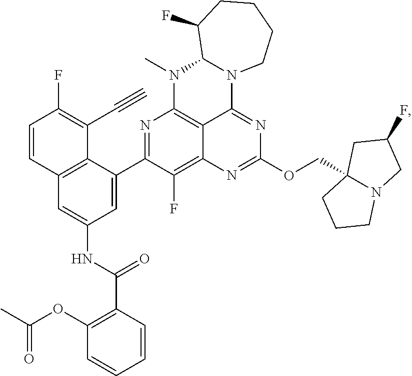 [SMILES: C#Cc1c(F)ccc2cc(NC(=O)c3ccccc3OC(C)=O)cc(-c3nc4c5c(nc(OC[C@@]67CCCN6C[C@H](F)C7)nc5c3F)N3CCCC[C@H](F)[C@H]3N4C)c12]

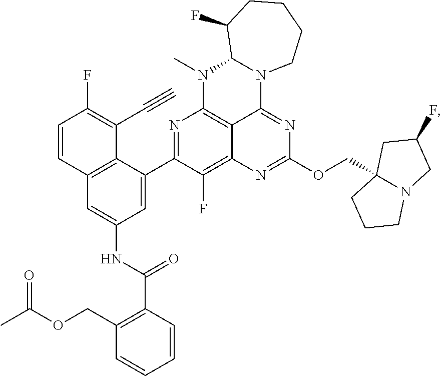 [SMILES: C#Cc1c(F)ccc2cc(NC(=O)c3ccccc3COC(C)=O)cc(-c3nc4c5c(nc(OC[C@@]67CCCN6C[C@H](F)C7)nc5c3F)N3CCCC[C@H](F)[C@H]3N4C)c12]

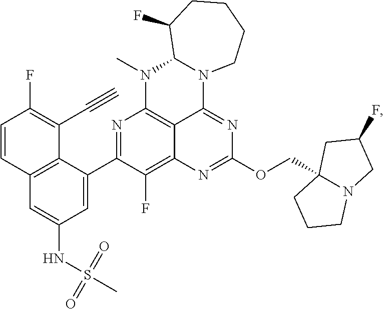 [SMILES: C#Cc1c(F)ccc2cc(NS(C)(=O)=O)cc(-c3nc4c5c(nc(OC[C@@]67CCCN6C[C@H](F)C7)nc5c3F)N3CCCC[C@H](F)[C@H]3N4C)c12]

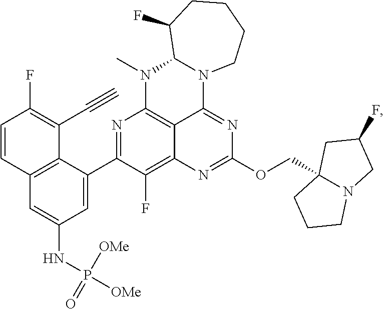 [SMILES: C#Cc1c(F)ccc2cc(NP(=O)(OC)OC)cc(-c3nc4c5c(nc(OC[C@@]67CCCN6C[C@H](F)C7)nc5c3F)N3CCCC[C@H](F)[C@H]3N4C)c12]

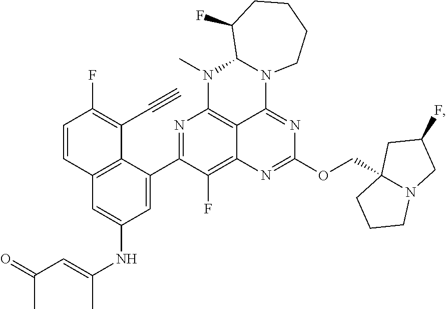 [SMILES: C#Cc1c(F)ccc2cc(N/C(C)=C/C(C)=O)cc(-c3nc4c5c(nc(OC[C@@]67CCCN6C[C@H](F)C7)nc5c3F)N3CCCC[C@H](F)[C@H]3N4C)c12]

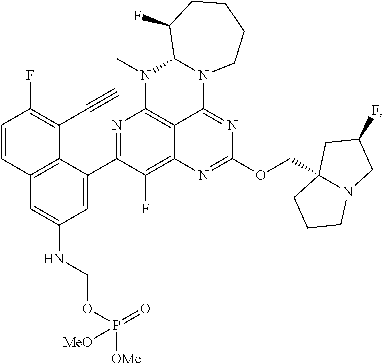 [SMILES: C#Cc1c(F)ccc2cc(NCOP(=O)(OC)OC)cc(-c3nc4c5c(nc(OC[C@@]67CCCN6C[C@H](F)C7)nc5c3F)N3CCCC[C@H](F)[C@H]3N4C)c12]

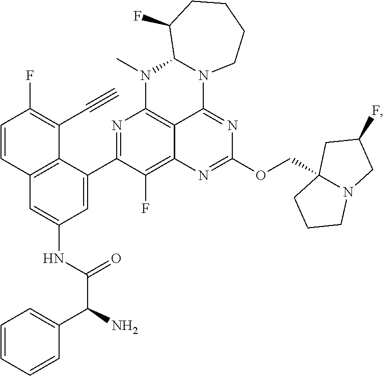 [SMILES: C#Cc1c(F)ccc2cc(NC(=O)[C@@H](N)c3ccccc3)cc(-c3nc4c5c(nc(OC[C@@]67CCCN6C[C@H](F)C7)nc5c3F)N3CCCC[C@H](F)[C@H]3N4C)c12]

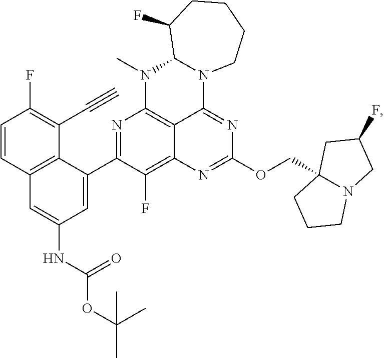 [SMILES: C#Cc1c(F)ccc2cc(NC(=O)OC(C)(C)C)cc(-c3nc4c5c(nc(OC[C@@]67CCCN6C[C@H](F)C7)nc5c3F)N3CCCC[C@H](F)[C@H]3N4C)c12]

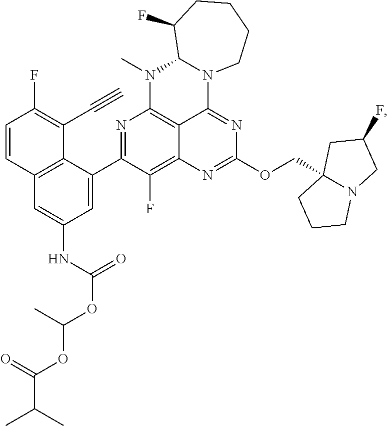 [SMILES: C#Cc1c(F)ccc2cc(NC(=O)OC(C)OC(=O)C(C)C)cc(-c3nc4c5c(nc(OC[C@@]67CCCN6C[C@H](F)C7)nc5c3F)N3CCCC[C@H](F)[C@H]3N4C)c12]

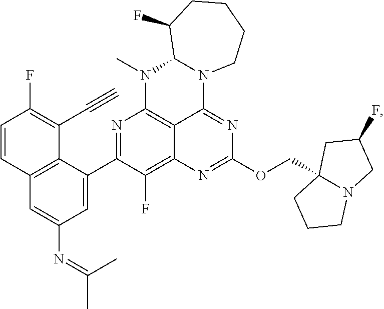 [SMILES: C#Cc1c(F)ccc2cc(N=C(C)C)cc(-c3nc4c5c(nc(OC[C@@]67CCCN6C[C@H](F)C7)nc5c3F)N3CCCC[C@H](F)[C@H]3N4C)c12]

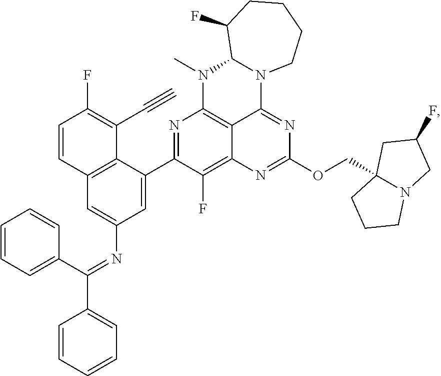 [SMILES: C#Cc1c(F)ccc2cc(N=C(c3ccccc3)c3ccccc3)cc(-c3nc4c5c(nc(OC[C@@]67CCCN6C[C@H](F)C7)nc5c3F)N3CCCC[C@H](F)[C@H]3N4C)c12]

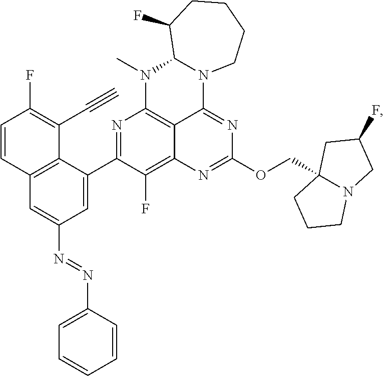 [SMILES: C#Cc1c(F)ccc2cc(/N=N/c3ccccc3)cc(-c3nc4c5c(nc(OC[C@@]67CCCN6C[C@H](F)C7)nc5c3F)N3CCCC[C@H](F)[C@H]3N4C)c12]

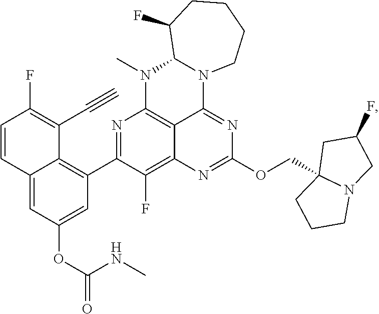 [SMILES: C#Cc1c(F)ccc2cc(OC(=O)NC)cc(-c3nc4c5c(nc(OC[C@@]67CCCN6C[C@H](F)C7)nc5c3F)N3CCCC[C@H](F)[C@H]3N4C)c12]

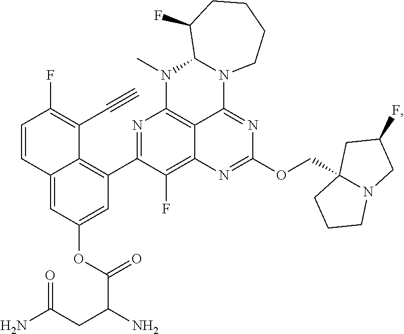 [SMILES: C#Cc1c(F)ccc2cc(OC(=O)C(N)CC(N)=O)cc(-c3nc4c5c(nc(OC[C@@]67CCCN6C[C@H](F)C7)nc5c3F)N3CCCC[C@H](F)[C@H]3N4C)c12]

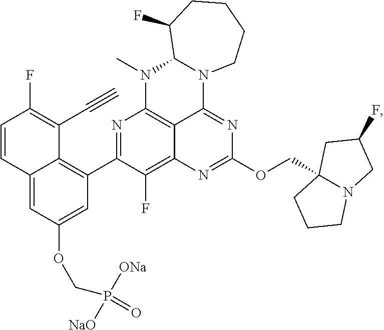

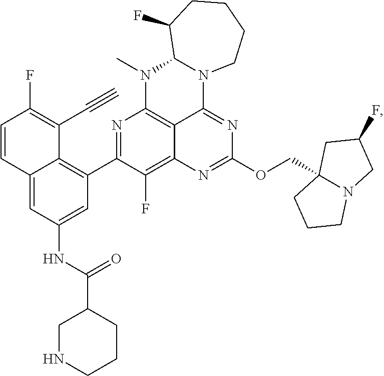 [SMILES: C#Cc1c(F)ccc2cc(NC(=O)C3CCCNC3)cc(-c3nc4c5c(nc(OC[C@@]67CCCN6C[C@H](F)C7)nc5c3F)N3CCCC[C@H](F)[C@H]3N4C)c12]

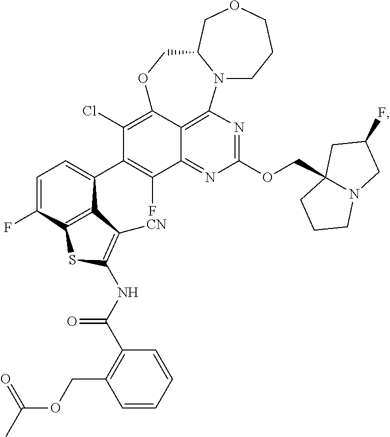

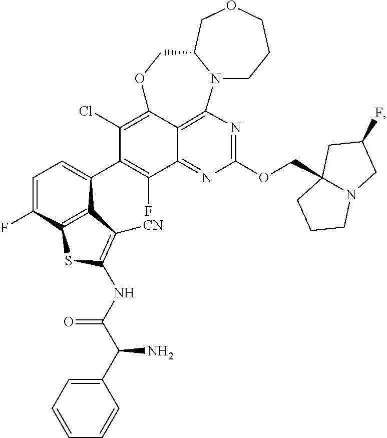

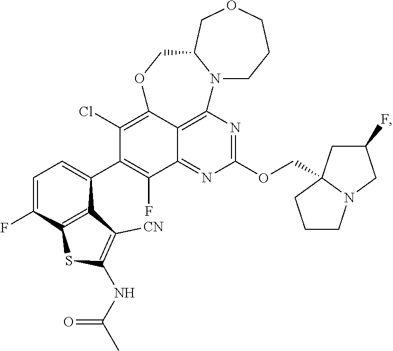

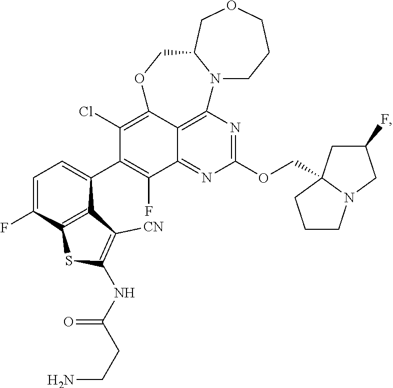

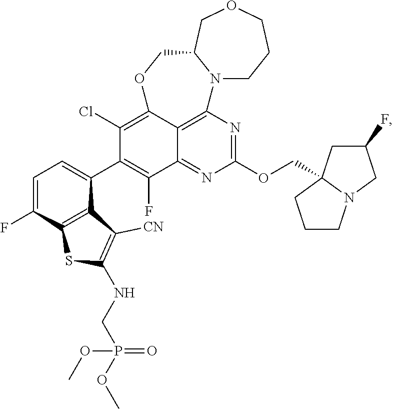

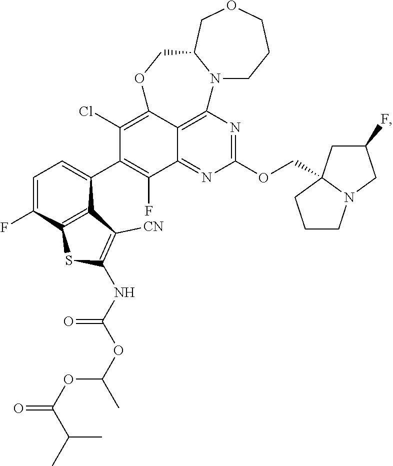

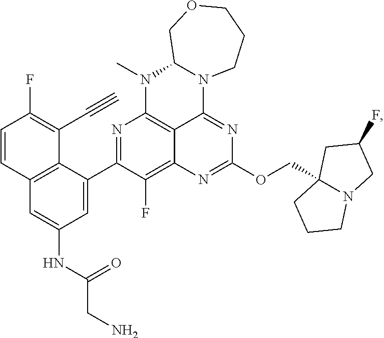 [SMILES: C#Cc1c(F)ccc2cc(NC(=O)CN)cc(-c3nc4c5c(nc(OC[C@@]67CCCN6C[C@H](F)C7)nc5c3F)N3CCCOC[C@H]3N4C)c12]

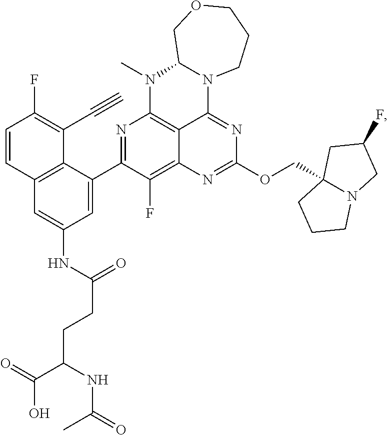 [SMILES: C#Cc1c(F)ccc2cc(NC(=O)CCC(NC(C)=O)C(=O)O)cc(-c3nc4c5c(nc(OC[C@@]67CCCN6C[C@H](F)C7)nc5c3F)N3CCCOC[C@H]3N4C)c12]

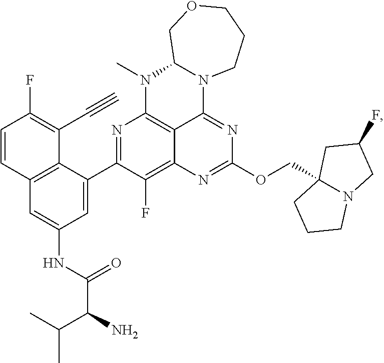 [SMILES: C#Cc1c(F)ccc2cc(NC(=O)[C@@H](N)C(C)C)cc(-c3nc4c5c(nc(OC[C@@]67CCCN6C[C@H](F)C7)nc5c3F)N3CCCOC[C@H]3N4C)c12]

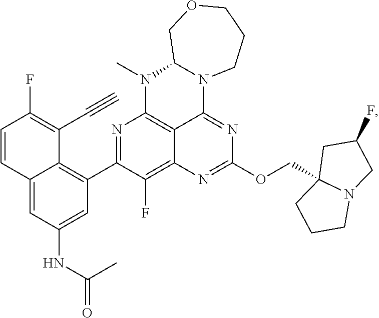 [SMILES: C#Cc1c(F)ccc2cc(NC(C)=O)cc(-c3nc4c5c(nc(OC[C@@]67CCCN6C[C@H](F)C7)nc5c3F)N3CCCOC[C@H]3N4C)c12]

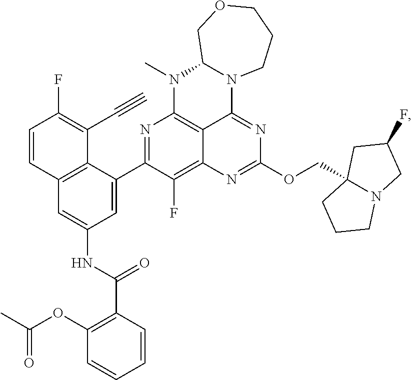 [SMILES: C#Cc1c(F)ccc2cc(NC(=O)c3ccccc3OC(C)=O)cc(-c3nc4c5c(nc(OC[C@@]67CCCN6C[C@H](F)C7)nc5c3F)N3CCCOC[C@H]3N4C)c12]

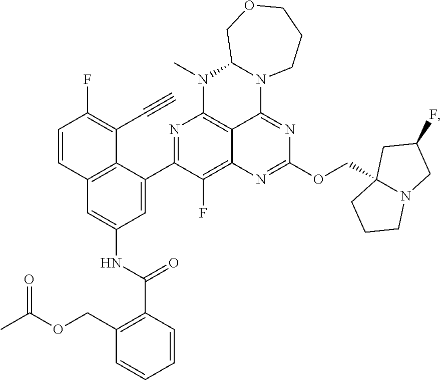 [SMILES: C#Cc1c(F)ccc2cc(NC(=O)c3ccccc3COC(C)=O)cc(-c3nc4c5c(nc(OC[C@@]67CCCN6C[C@H](F)C7)nc5c3F)N3CCCOC[C@H]3N4C)c12]

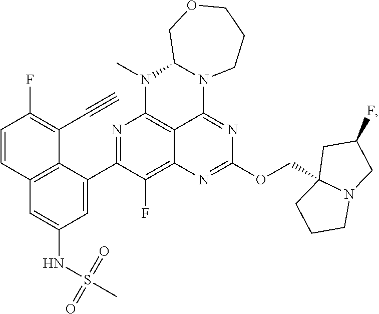 [SMILES: C#Cc1c(F)ccc2cc(NS(C)(=O)=O)cc(-c3nc4c5c(nc(OC[C@@]67CCCN6C[C@H](F)C7)nc5c3F)N3CCCOC[C@H]3N4C)c12]

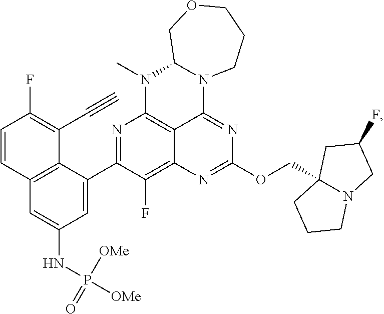 [SMILES: C#Cc1c(F)ccc2cc(NP(=O)(OC)OC)cc(-c3nc4c5c(nc(OC[C@@]67CCCN6C[C@H](F)C7)nc5c3F)N3CCCOC[C@H]3N4C)c12]

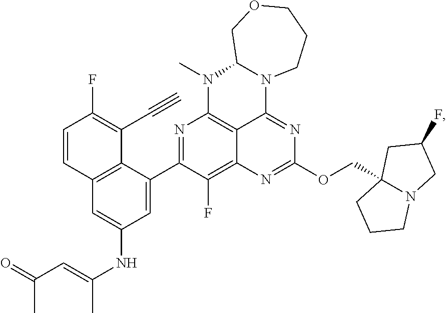 [SMILES: C#Cc1c(F)ccc2cc(N/C(C)=C/C(C)=O)cc(-c3nc4c5c(nc(OC[C@@]67CCCN6C[C@H](F)C7)nc5c3F)N3CCCOC[C@H]3N4C)c12]

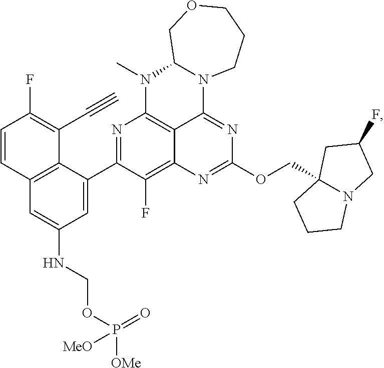 [SMILES: C#Cc1c(F)ccc2cc(NCOP(=O)(OC)OC)cc(-c3nc4c5c(nc(OC[C@@]67CCCN6C[C@H](F)C7)nc5c3F)N3CCCOC[C@H]3N4C)c12]

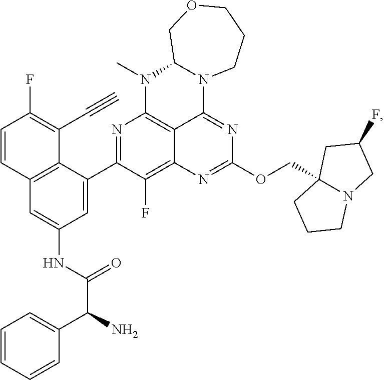 [SMILES: C#Cc1c(F)ccc2cc(NC(=O)[C@@H](N)c3ccccc3)cc(-c3nc4c5c(nc(OC[C@@]67CCCN6C[C@H](F)C7)nc5c3F)N3CCCOC[C@H]3N4C)c12]

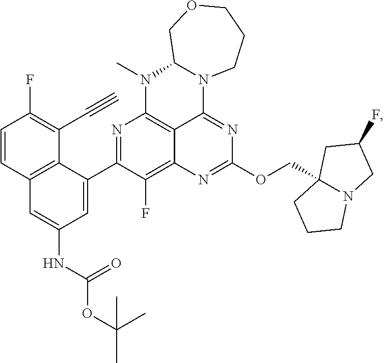 [SMILES: C#Cc1c(F)ccc2cc(NC(=O)OC(C)(C)C)cc(-c3nc4c5c(nc(OC[C@@]67CCCN6C[C@H](F)C7)nc5c3F)N3CCCOC[C@H]3N4C)c12]

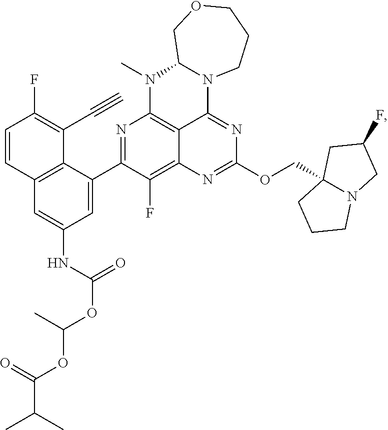 [SMILES: C#Cc1c(F)ccc2cc(NC(=O)OC(C)OC(=O)C(C)C)cc(-c3nc4c5c(nc(OC[C@@]67CCCN6C[C@H](F)C7)nc5c3F)N3CCCOC[C@H]3N4C)c12]

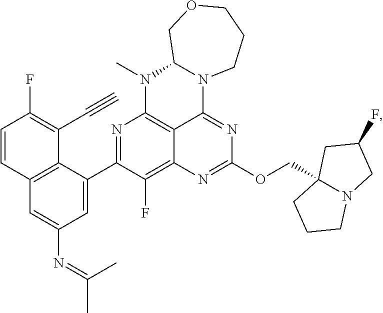 [SMILES: C#Cc1c(F)ccc2cc(N=C(C)C)cc(-c3nc4c5c(nc(OC[C@@]67CCCN6C[C@H](F)C7)nc5c3F)N3CCCOC[C@H]3N4C)c12]

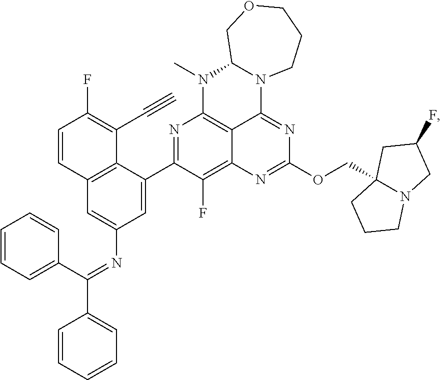 [SMILES: C#Cc1c(F)ccc2cc(N=C(c3ccccc3)c3ccccc3)cc(-c3nc4c5c(nc(OC[C@@]67CCCN6C[C@H](F)C7)nc5c3F)N3CCCOC[C@H]3N4C)c12]

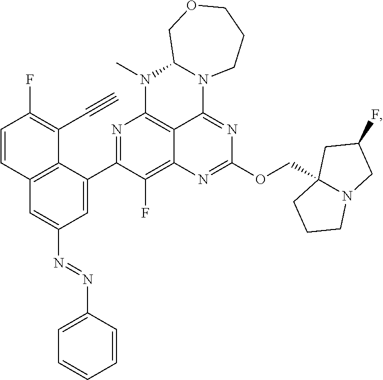 [SMILES: C#Cc1c(F)ccc2cc(/N=N/c3ccccc3)cc(-c3nc4c5c(nc(OC[C@@]67CCCN6C[C@H](F)C7)nc5c3F)N3CCCOC[C@H]3N4C)c12]

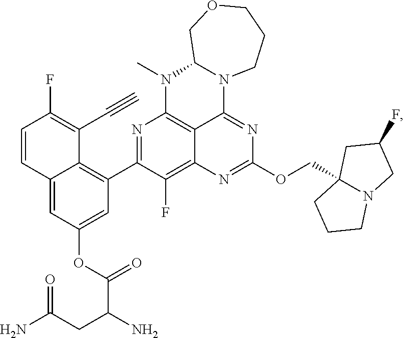 [SMILES: C#Cc1c(F)ccc2cc(OC(=O)C(N)CC(N)=O)cc(-c3nc4c5c(nc(OC[C@@]67CCCN6C[C@H](F)C7)nc5c3F)N3CCCOC[C@H]3N4C)c12]

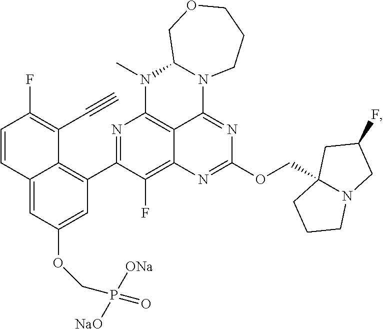

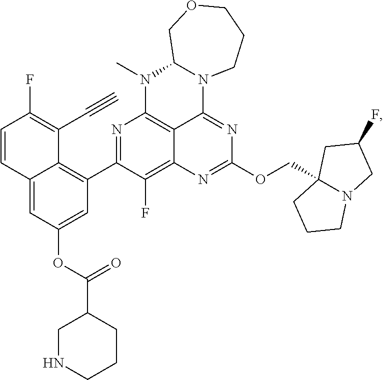 [SMILES: C#Cc1c(F)ccc2cc(OC(=O)C3CCCNC3)cc(-c3nc4c5c(nc(OC[C@@]67CCCN6C[C@H](F)C7)nc5c3F)N3CCCOC[C@H]3N4C)c12]

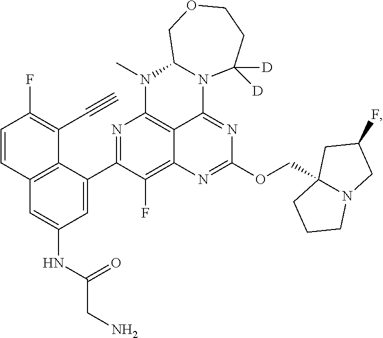 [SMILES: [2H]C1([2H])CCOC[C@H]2N(C)c3nc(-c4cc(NC(=O)CN)cc5ccc(F)c(C#C)c45)c(F)c4nc(OC[C@@]56CCCN5C[C@H](F)C6)nc(c34)N21]

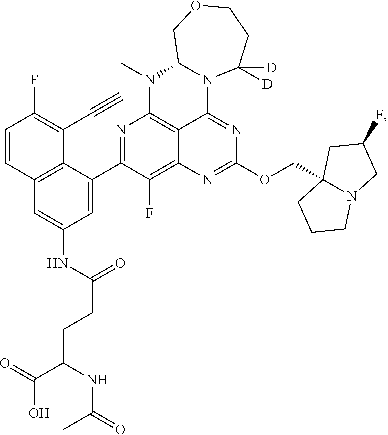 [SMILES: [2H]C1([2H])CCOC[C@H]2N(C)c3nc(-c4cc(NC(=O)CCC(NC(C)=O)C(=O)O)cc5ccc(F)c(C#C)c45)c(F)c4nc(OC[C@@]56CCCN5C[C@H](F)C6)nc(c34)N21]

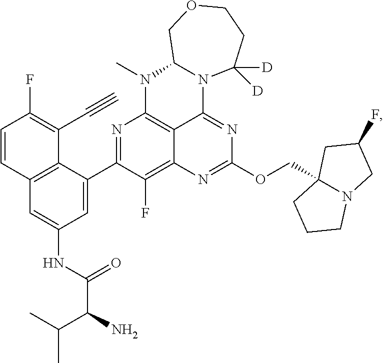 [SMILES: [2H]C1([2H])CCOC[C@H]2N(C)c3nc(-c4cc(NC(=O)[C@@H](N)C(C)C)cc5ccc(F)c(C#C)c45)c(F)c4nc(OC[C@@]56CCCN5C[C@H](F)C6)nc(c34)N21]

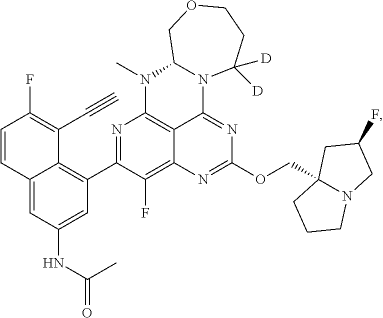 [SMILES: [2H]C1([2H])CCOC[C@H]2N(C)c3nc(-c4cc(NC(C)=O)cc5ccc(F)c(C#C)c45)c(F)c4nc(OC[C@@]56CCCN5C[C@H](F)C6)nc(c34)N21]

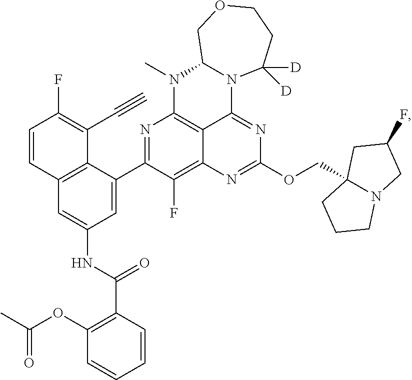 [SMILES: [2H]C1([2H])CCOC[C@H]2N(C)c3nc(-c4cc(NC(=O)c5ccccc5OC(C)=O)cc5ccc(F)c(C#C)c45)c(F)c4nc(OC[C@@]56CCCN5C[C@H](F)C6)nc(c34)N21]

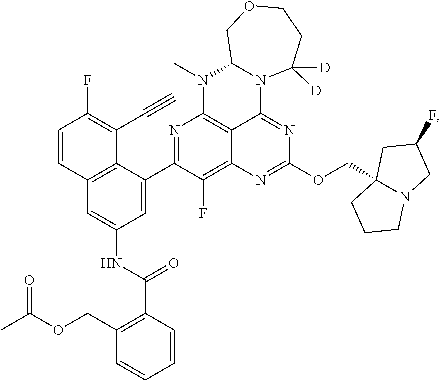 [SMILES: [2H]C1([2H])CCOC[C@H]2N(C)c3nc(-c4cc(NC(=O)c5ccccc5COC(C)=O)cc5ccc(F)c(C#C)c45)c(F)c4nc(OC[C@@]56CCCN5C[C@H](F)C6)nc(c34)N21]

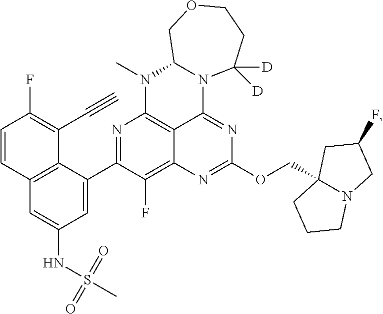 [SMILES: [2H]C1([2H])CCOC[C@H]2N(C)c3nc(-c4cc(NS(C)(=O)=O)cc5ccc(F)c(C#C)c45)c(F)c4nc(OC[C@@]56CCCN5C[C@H](F)C6)nc(c34)N21]

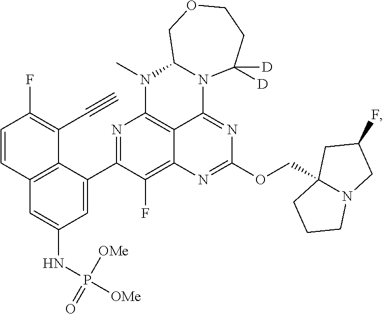 [SMILES: [2H]C1([2H])CCOC[C@H]2N(C)c3nc(-c4cc(NP(=O)(OC)OC)cc5ccc(F)c(C#C)c45)c(F)c4nc(OC[C@@]56CCCN5C[C@H](F)C6)nc(c34)N21]

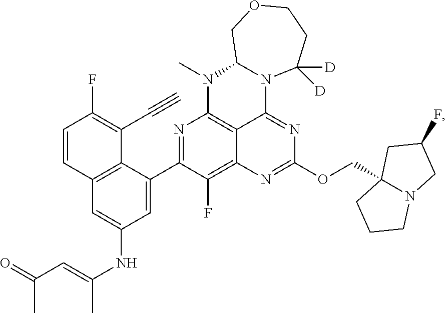 [SMILES: [2H]C1([2H])CCOC[C@H]2N(C)c3nc(-c4cc(N/C(C)=C/C(C)=O)cc5ccc(F)c(C#C)c45)c(F)c4nc(OC[C@@]56CCCN5C[C@H](F)C6)nc(c34)N21]

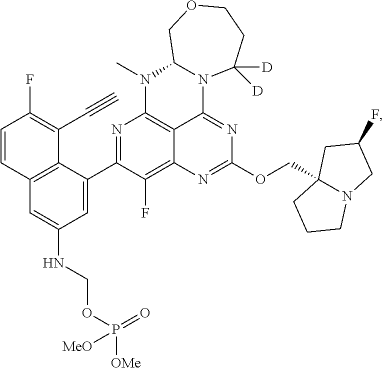 [SMILES: [2H]C1([2H])CCOC[C@H]2N(C)c3nc(-c4cc(NCOP(=O)(OC)OC)cc5ccc(F)c(C#C)c45)c(F)c4nc(OC[C@@]56CCCN5C[C@H](F)C6)nc(c34)N21]

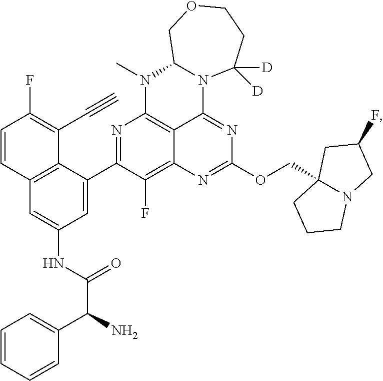 [SMILES: [2H]C1([2H])CCOC[C@H]2N(C)c3nc(-c4cc(NC(=O)[C@@H](N)c5ccccc5)cc5ccc(F)c(C#C)c45)c(F)c4nc(OC[C@@]56CCCN5C[C@H](F)C6)nc(c34)N21]

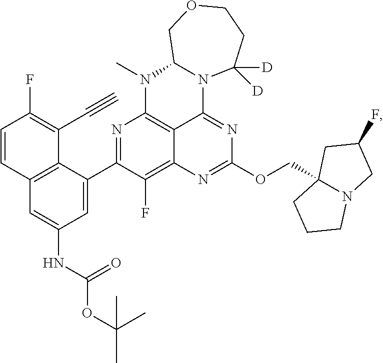 [SMILES: [2H]C1([2H])CCOC[C@H]2N(C)c3nc(-c4cc(NC(=O)OC(C)(C)C)cc5ccc(F)c(C#C)c45)c(F)c4nc(OC[C@@]56CCCN5C[C@H](F)C6)nc(c34)N21]

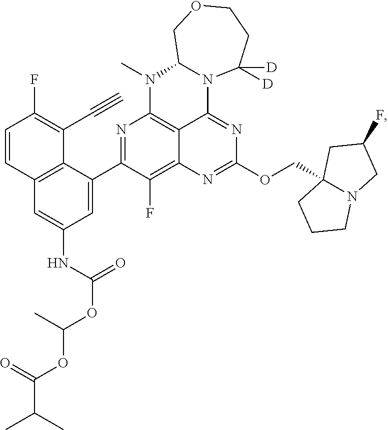 [SMILES: [2H]C1([2H])CCOC[C@H]2N(C)c3nc(-c4cc(NC(=O)OC(C)OC(=O)C(C)C)cc5ccc(F)c(C#C)c45)c(F)c4nc(OC[C@@]56CCCN5C[C@H](F)C6)nc(c34)N21]

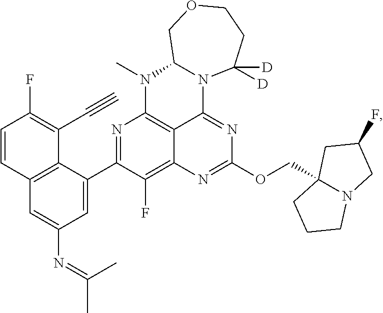 [SMILES: [2H]C1([2H])CCOC[C@H]2N(C)c3nc(-c4cc(N=C(C)C)cc5ccc(F)c(C#C)c45)c(F)c4nc(OC[C@@]56CCCN5C[C@H](F)C6)nc(c34)N21]

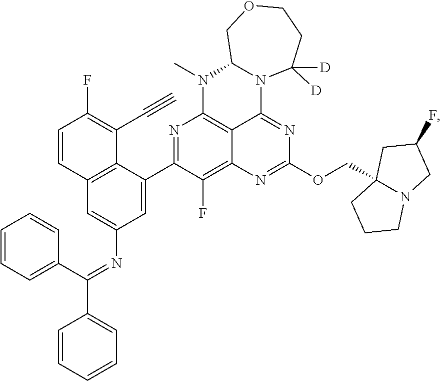 [SMILES: [2H]C1([2H])CCOC[C@H]2N(C)c3nc(-c4cc(N=C(c5ccccc5)c5ccccc5)cc5ccc(F)c(C#C)c45)c(F)c4nc(OC[C@@]56CCCN5C[C@H](F)C6)nc(c34)N21]

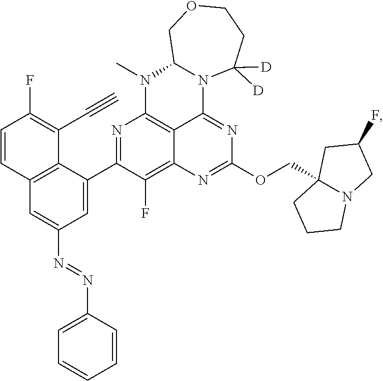 [SMILES: [2H]C1([2H])CCOC[C@H]2N(C)c3nc(-c4cc(/N=N/c5ccccc5)cc5ccc(F)c(C#C)c45)c(F)c4nc(OC[C@@]56CCCN5C[C@H](F)C6)nc(c34)N21]

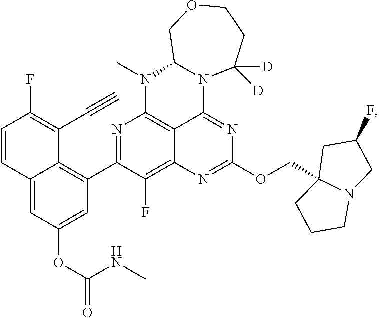 [SMILES: [2H]C1([2H])CCOC[C@H]2N(C)c3nc(-c4cc(OC(=O)NC)cc5ccc(F)c(C#C)c45)c(F)c4nc(OC[C@@]56CCCN5C[C@H](F)C6)nc(c34)N21]

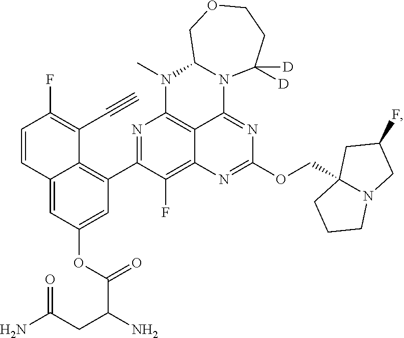 [SMILES: [2H]C1([2H])CCOC[C@H]2N(C)c3nc(-c4cc(OC(=O)C(N)CC(N)=O)cc5ccc(F)c(C#C)c45)c(F)c4nc(OC[C@@]56CCCN5C[C@H](F)C6)nc(c34)N21]

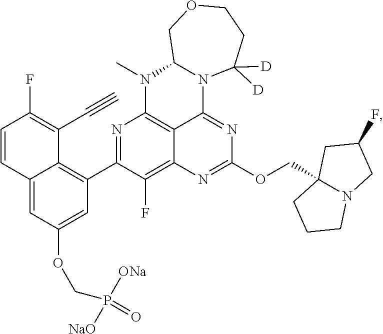

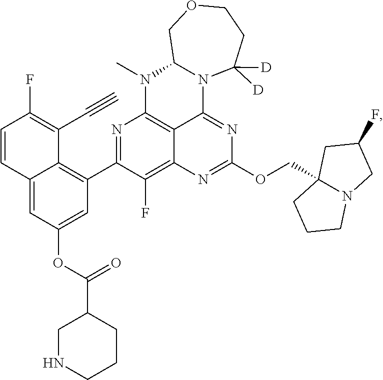 [SMILES: [2H]C1([2H])CCOC[C@H]2N(C)c3nc(-c4cc(OC(=O)C5CCCNC5)cc5ccc(F)c(C#C)c45)c(F)c4nc(OC[C@@]56CCCN5C[C@H](F)C6)nc(c34)N21]

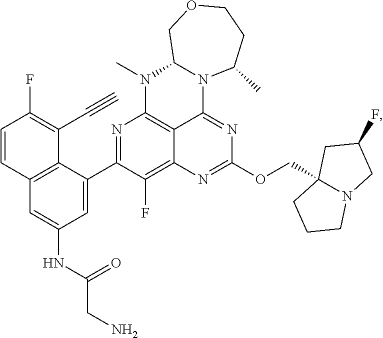 [SMILES: C#Cc1c(F)ccc2cc(NC(=O)CN)cc(-c3nc4c5c(nc(OC[C@@]67CCCN6C[C@H](F)C7)nc5c3F)N3[C@@H](C)CCOC[C@H]3N4C)c12]

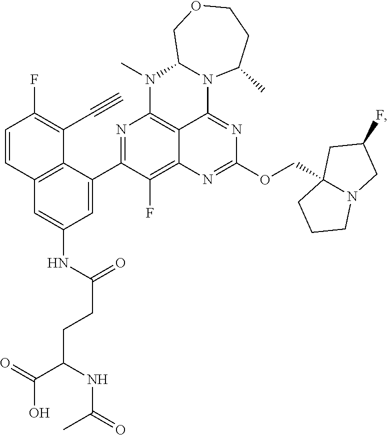 [SMILES: C#Cc1c(F)ccc2cc(NC(=O)CCC(NC(C)=O)C(=O)O)cc(-c3nc4c5c(nc(OC[C@@]67CCCN6C[C@H](F)C7)nc5c3F)N3[C@@H](C)CCOC[C@H]3N4C)c12]

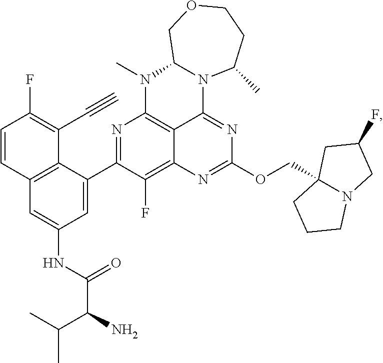 [SMILES: C#Cc1c(F)ccc2cc(NC(=O)[C@@H](N)C(C)C)cc(-c3nc4c5c(nc(OC[C@@]67CCCN6C[C@H](F)C7)nc5c3F)N3[C@@H](C)CCOC[C@H]3N4C)c12]

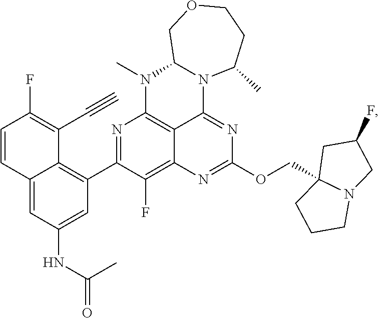 [SMILES: C#Cc1c(F)ccc2cc(NC(C)=O)cc(-c3nc4c5c(nc(OC[C@@]67CCCN6C[C@H](F)C7)nc5c3F)N3[C@@H](C)CCOC[C@H]3N4C)c12]

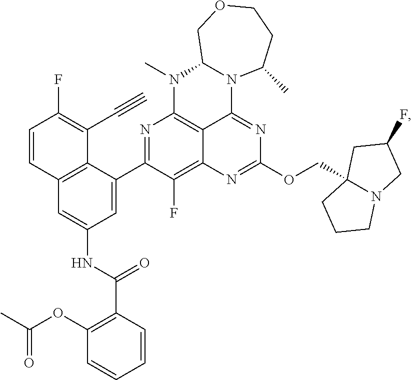 [SMILES: C#Cc1c(F)ccc2cc(NC(=O)c3ccccc3OC(C)=O)cc(-c3nc4c5c(nc(OC[C@@]67CCCN6C[C@H](F)C7)nc5c3F)N3[C@@H](C)CCOC[C@H]3N4C)c12]

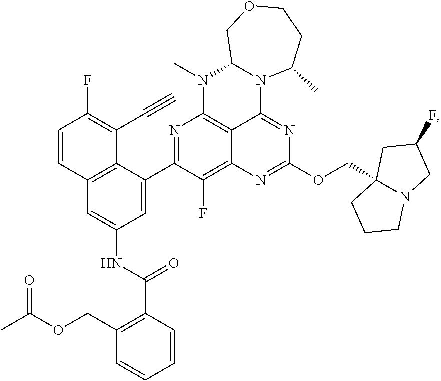 [SMILES: C#Cc1c(F)ccc2cc(NC(=O)c3ccccc3COC(C)=O)cc(-c3nc4c5c(nc(OC[C@@]67CCCN6C[C@H](F)C7)nc5c3F)N3[C@@H](C)CCOC[C@H]3N4C)c12]

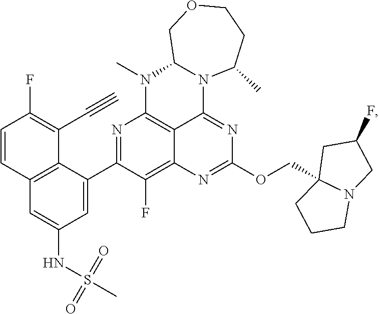 [SMILES: C#Cc1c(F)ccc2cc(NS(C)(=O)=O)cc(-c3nc4c5c(nc(OC[C@@]67CCCN6C[C@H](F)C7)nc5c3F)N3[C@@H](C)CCOC[C@H]3N4C)c12]

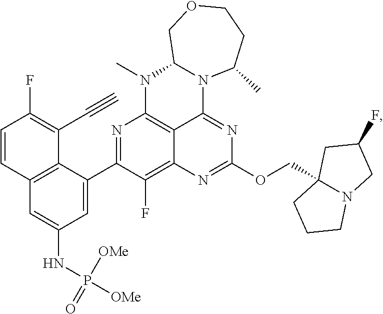 [SMILES: C#Cc1c(F)ccc2cc(NP(=O)(OC)OC)cc(-c3nc4c5c(nc(OC[C@@]67CCCN6C[C@H](F)C7)nc5c3F)N3[C@@H](C)CCOC[C@H]3N4C)c12]

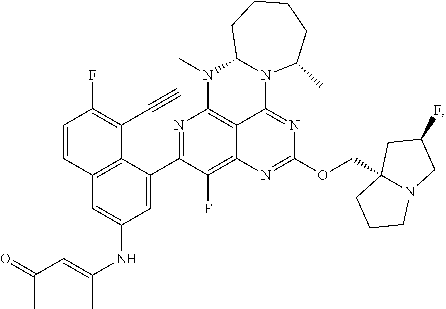 [SMILES: C#Cc1c(F)ccc2cc(N/C(C)=C/C(C)=O)cc(-c3nc4c5c(nc(OC[C@@]67CCCN6C[C@H](F)C7)nc5c3F)N3[C@@H](C)CCCC[C@H]3N4C)c12]

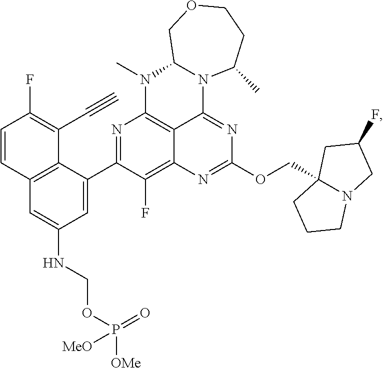 [SMILES: C#Cc1c(F)ccc2cc(NCOP(=O)(OC)OC)cc(-c3nc4c5c(nc(OC[C@@]67CCCN6C[C@H](F)C7)nc5c3F)N3[C@@H](C)CCOC[C@H]3N4C)c12]

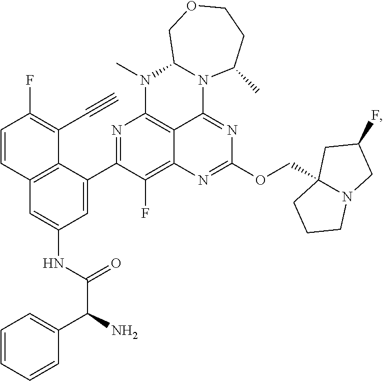 [SMILES: C#Cc1c(F)ccc2cc(NC(=O)[C@@H](N)c3ccccc3)cc(-c3nc4c5c(nc(OC[C@@]67CCCN6C[C@H](F)C7)nc5c3F)N3[C@@H](C)CCOC[C@H]3N4C)c12]

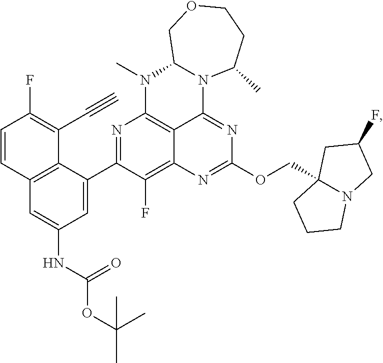 [SMILES: C#Cc1c(F)ccc2cc(NC(=O)OC(C)(C)C)cc(-c3nc4c5c(nc(OC[C@@]67CCCN6C[C@H](F)C7)nc5c3F)N3[C@@H](C)CCOC[C@H]3N4C)c12]

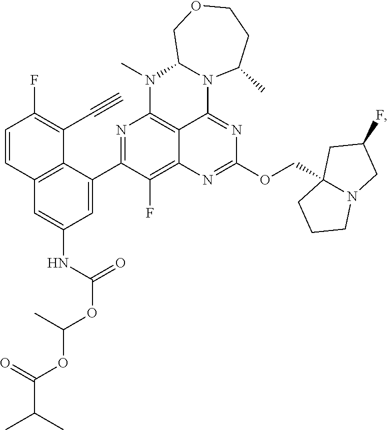 [SMILES: C#Cc1c(F)ccc2cc(NC(=O)OC(C)OC(=O)C(C)C)cc(-c3nc4c5c(nc(OC[C@@]67CCCN6C[C@H](F)C7)nc5c3F)N3[C@@H](C)CCOC[C@H]3N4C)c12]

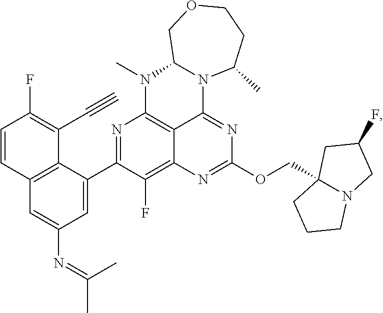 [SMILES: C#Cc1c(F)ccc2cc(N=C(C)C)cc(-c3nc4c5c(nc(OC[C@@]67CCCN6C[C@H](F)C7)nc5c3F)N3[C@@H](C)CCOC[C@H]3N4C)c12]

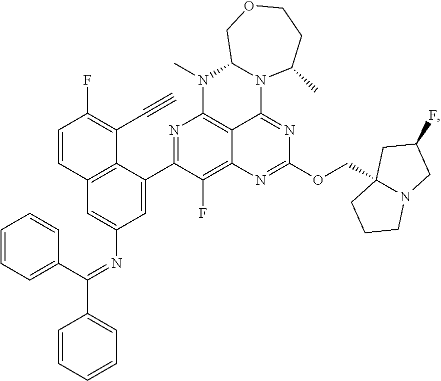 [SMILES: C#Cc1c(F)ccc2cc(N=C(c3ccccc3)c3ccccc3)cc(-c3nc4c5c(nc(OC[C@@]67CCCN6C[C@H](F)C7)nc5c3F)N3[C@@H](C)CCOC[C@H]3N4C)c12]

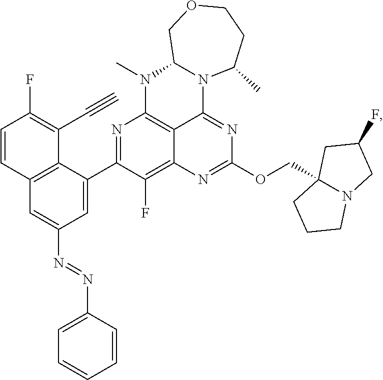 [SMILES: C#Cc1c(F)ccc2cc(/N=N/c3ccccc3)cc(-c3nc4c5c(nc(OC[C@@]67CCCN6C[C@H](F)C7)nc5c3F)N3[C@@H](C)CCOC[C@H]3N4C)c12]

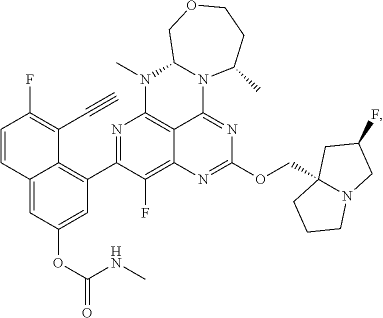 [SMILES: C#Cc1c(F)ccc2cc(OC(=O)NC)cc(-c3nc4c5c(nc(OC[C@@]67CCCN6C[C@H](F)C7)nc5c3F)N3[C@@H](C)CCOC[C@H]3N4C)c12]

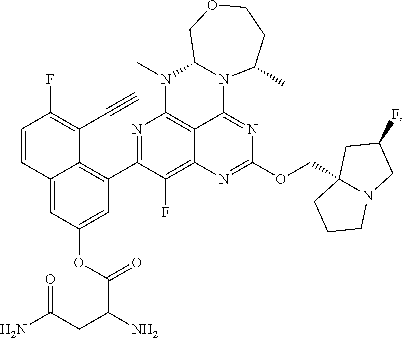 [SMILES: C#Cc1c(F)ccc2cc(OC(=O)C(N)CC(N)=O)cc(-c3nc4c5c(nc(OC[C@@]67CCCN6C[C@H](F)C7)nc5c3F)N3[C@@H](C)CCOC[C@H]3N4C)c12]

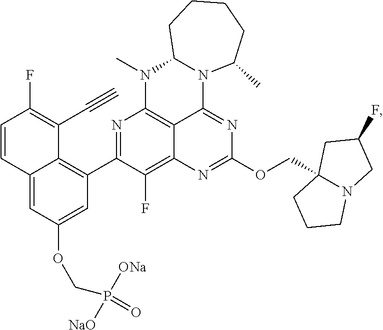

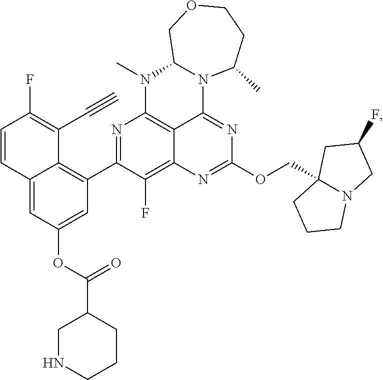 [SMILES: C#Cc1c(F)ccc2cc(OC(=O)C3CCCNC3)cc(-c3nc4c5c(nc(OC[C@@]67CCCN6C[C@H](F)C7)nc5c3F)N3[C@@H](C)CCOC[C@H]3N4C)c12]

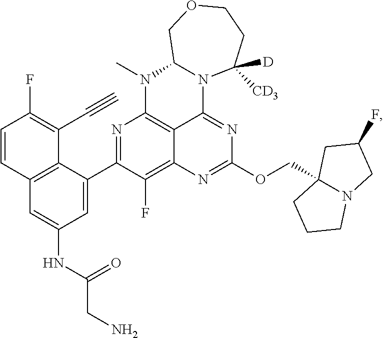 [SMILES: [2H]C([2H])([2H])[C@@]1([2H])CCOC[C@H]2N(C)c3nc(-c4cc(NC(=O)CN)cc5ccc(F)c(C#C)c45)c(F)c4nc(OC[C@@]56CCCN5C[C@H](F)C6)nc(c34)N21]

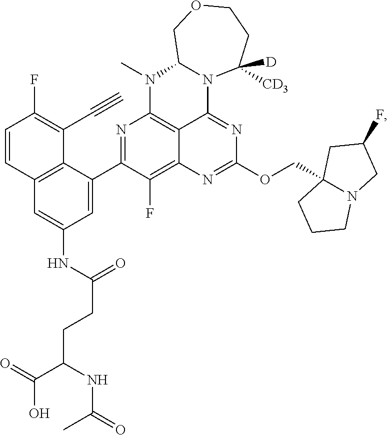 [SMILES: [2H]C([2H])([2H])[C@@]1([2H])CCOC[C@H]2N(C)c3nc(-c4cc(NC(=O)CCC(NC(C)=O)C(=O)O)cc5ccc(F)c(C#C)c45)c(F)c4nc(OC[C@@]56CCCN5C[C@H](F)C6)nc(c34)N21]

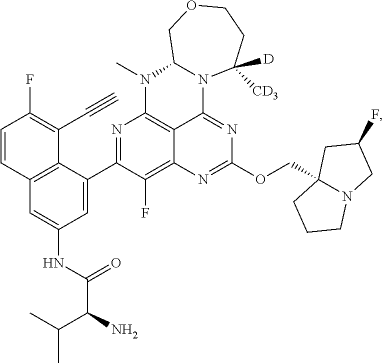 [SMILES: [2H]C([2H])([2H])[C@@]1([2H])CCOC[C@H]2N(C)c3nc(-c4cc(NC(=O)[C@@H](N)C(C)C)cc5ccc(F)c(C#C)c45)c(F)c4nc(OC[C@@]56CCCN5C[C@H](F)C6)nc(c34)N21]

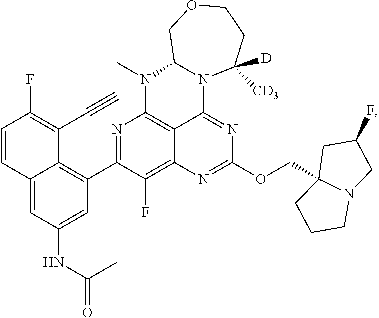 [SMILES: [2H]C([2H])([2H])[C@@]1([2H])CCOC[C@H]2N(C)c3nc(-c4cc(NC(C)=O)cc5ccc(F)c(C#C)c45)c(F)c4nc(OC[C@@]56CCCN5C[C@H](F)C6)nc(c34)N21]

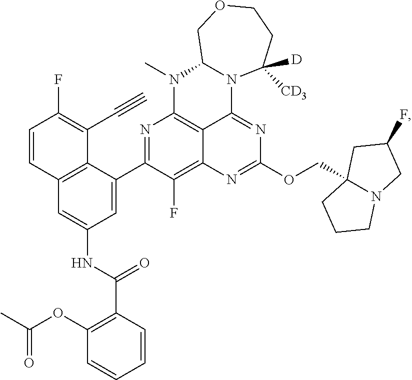 [SMILES: [2H]C([2H])([2H])[C@@]1([2H])CCOC[C@H]2N(C)c3nc(-c4cc(NC(=O)c5ccccc5OC(C)=O)cc5ccc(F)c(C#C)c45)c(F)c4nc(OC[C@@]56CCCN5C[C@H](F)C6)nc(c34)N21]

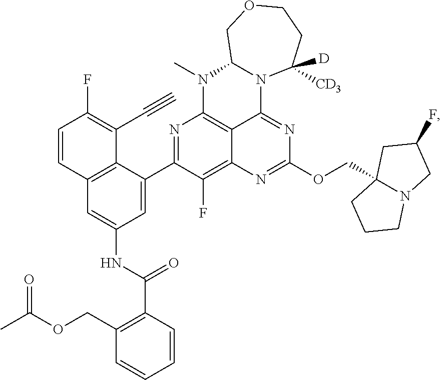 [SMILES: [2H]C([2H])([2H])[C@@]1([2H])CCOC[C@H]2N(C)c3nc(-c4cc(NC(=O)c5ccccc5COC(C)=O)cc5ccc(F)c(C#C)c45)c(F)c4nc(OC[C@@]56CCCN5C[C@H](F)C6)nc(c34)N21]

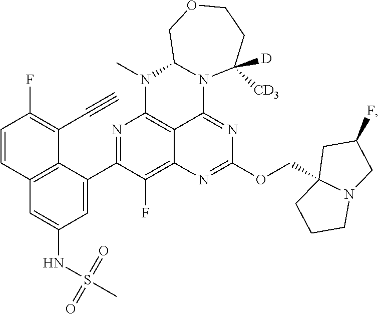 [SMILES: [2H]C([2H])([2H])[C@@]1([2H])CCOC[C@H]2N(C)c3nc(-c4cc(NS(C)(=O)=O)cc5ccc(F)c(C#C)c45)c(F)c4nc(OC[C@@]56CCCN5C[C@H](F)C6)nc(c34)N21]

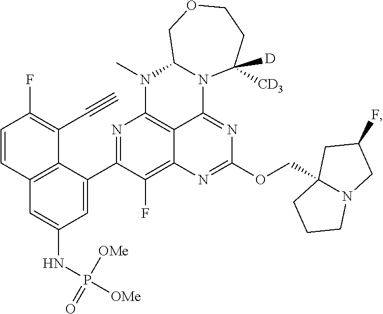 [SMILES: [2H]C([2H])([2H])[C@@]1([2H])CCOC[C@H]2N(C)c3nc(-c4cc(NP(=O)(OC)OC)cc5ccc(F)c(C#C)c45)c(F)c4nc(OC[C@@]56CCCN5C[C@H](F)C6)nc(c34)N21]

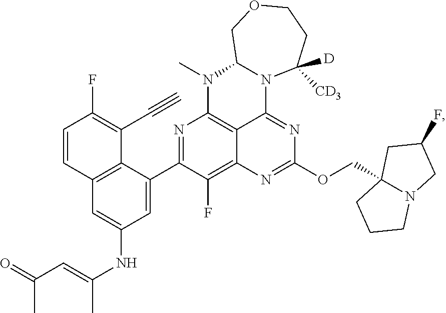 [SMILES: [2H]C([2H])([2H])[C@@]1([2H])CCOC[C@H]2N(C)c3nc(-c4cc(N/C(C)=C/C(C)=O)cc5ccc(F)c(C#C)c45)c(F)c4nc(OC[C@@]56CCCN5C[C@H](F)C6)nc(c34)N21]

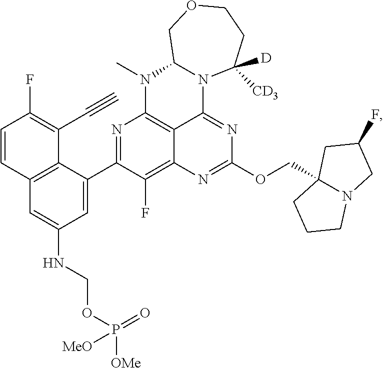 [SMILES: [2H]C([2H])([2H])[C@@]1([2H])CCOC[C@H]2N(C)c3nc(-c4cc(NCOP(=O)(OC)OC)cc5ccc(F)c(C#C)c45)c(F)c4nc(OC[C@@]56CCCN5C[C@H](F)C6)nc(c34)N21]

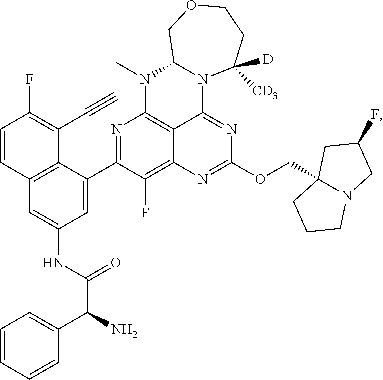 [SMILES: [2H]C([2H])([2H])[C@@]1([2H])CCOC[C@H]2N(C)c3nc(-c4cc(NC(=O)[C@@H](N)c5ccccc5)cc5ccc(F)c(C#C)c45)c(F)c4nc(OC[C@@]56CCCN5C[C@H](F)C6)nc(c34)N21]

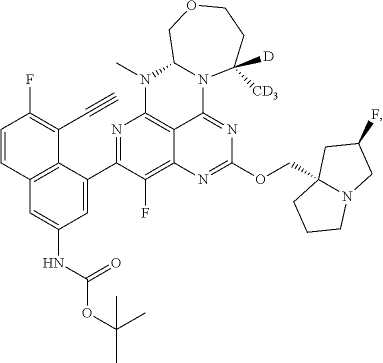 [SMILES: [2H]C([2H])([2H])[C@@]1([2H])CCOC[C@H]2N(C)c3nc(-c4cc(NC(=O)OC(C)(C)C)cc5ccc(F)c(C#C)c45)c(F)c4nc(OC[C@@]56CCCN5C[C@H](F)C6)nc(c34)N21]

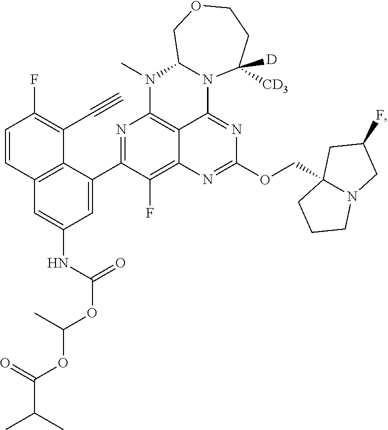 [SMILES: [2H]C([2H])([2H])[C@@]1([2H])CCOC[C@H]2N(C)c3nc(-c4cc(NC(=O)OC(C)OC(=O)C(C)C)cc5ccc(F)c(C#C)c45)c(F)c4nc(OC[C@@]56CCCN5C[C@H](F)C6)nc(c34)N21]

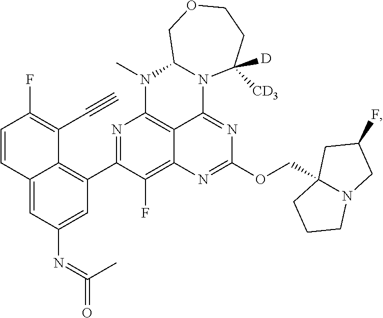

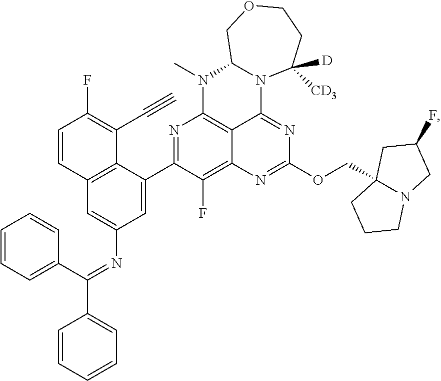 [SMILES: [2H]C([2H])([2H])[C@@]1([2H])CCOC[C@H]2N(C)c3nc(-c4cc(N=C(c5ccccc5)c5ccccc5)cc5ccc(F)c(C#C)c45)c(F)c4nc(OC[C@@]56CCCN5C[C@H](F)C6)nc(c34)N21]

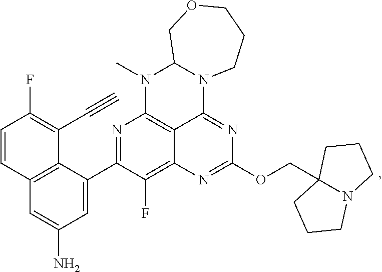 [SMILES: C#Cc1c(F)ccc2cc(N)cc(-c3nc4c5c(nc(OCC67CCCN6CCC7)nc5c3F)N3CCCOCC3N4C)c12]

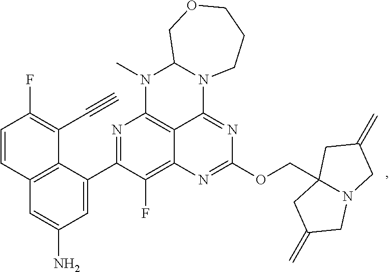 [SMILES: C#Cc1c(F)ccc2cc(N)cc(-c3nc4c5c(nc(OCC67CC(=C)CN6CC(=C)C7)nc5c3F)N3CCCOCC3N4C)c12]

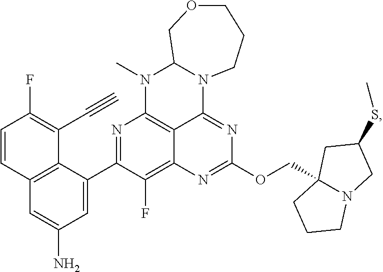 [SMILES: C#Cc1c(F)ccc2cc(N)cc(-c3nc4c5c(nc(OC[C@@]67CCCN6C[C@H](SC)C7)nc5c3F)N3CCCOCC3N4C)c12]

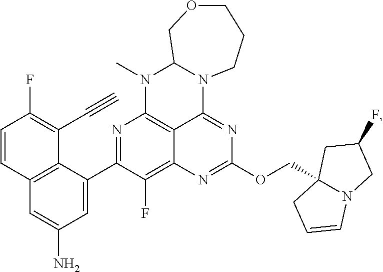 [SMILES: C#Cc1c(F)ccc2cc(N)cc(-c3nc4c5c(nc(OC[C@@]67CC=CN6C[C@H](F)C7)nc5c3F)N3CCCOCC3N4C)c12]

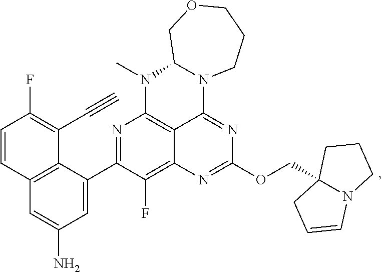 [SMILES: C#Cc1c(F)ccc2cc(N)cc(-c3nc4c5c(nc(OC[C@@]67CC=CN6CCC7)nc5c3F)N3CCCOC[C@H]3N4C)c12]

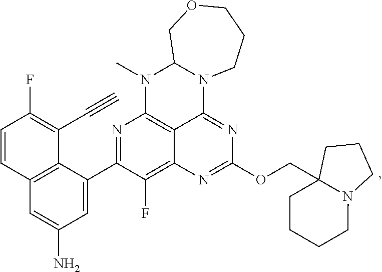 [SMILES: C#Cc1c(F)ccc2cc(N)cc(-c3nc4c5c(nc(OCC67CCCCN6CCC7)nc5c3F)N3CCCOCC3N4C)c12]

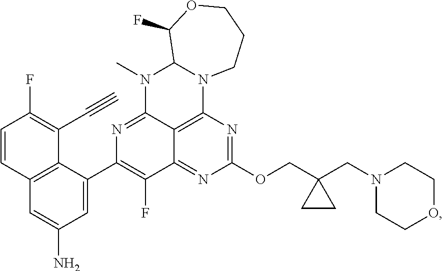 [SMILES: C#Cc1c(F)ccc2cc(N)cc(-c3nc4c5c(nc(OCC6(CN7CCOCC7)CC6)nc5c3F)N3CCCO[C@H](F)C3N4C)c12]

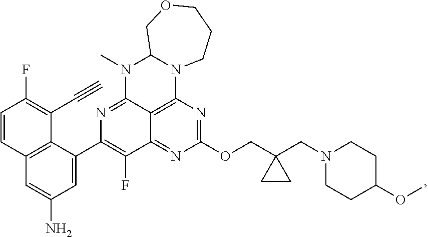

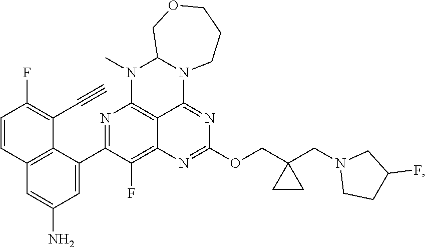 [SMILES: C#Cc1c(F)ccc2cc(N)cc(-c3nc4c5c(nc(OCC6(CN7CCC(F)C7)CC6)nc5c3F)N3CCCOCC3N4C)c12]

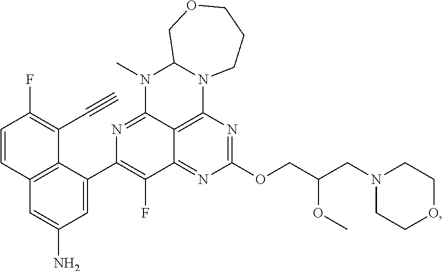 [SMILES: C#Cc1c(F)ccc2cc(N)cc(-c3nc4c5c(nc(OCC(CN6CCOCC6)OC)nc5c3F)N3CCCOCC3N4C)c12]

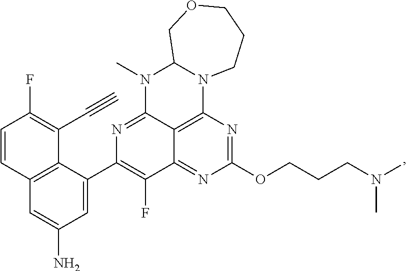

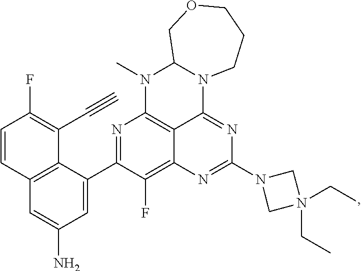

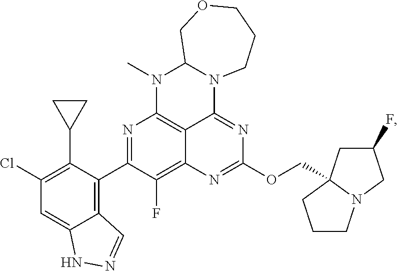 [SMILES: CN1c2nc(-c3c(C4CC4)c(Cl)cc4[nH]ncc34)c(F)c3nc(OC[C@@]45CCCN4C[C@H](F)C5)nc(c23)N2CCCOCC12]

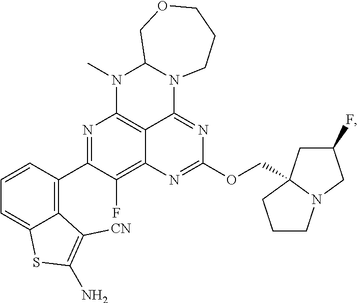 [SMILES: CN1c2nc(-c3cccc4sc(N)c(C#N)c34)c(F)c3nc(OC[C@@]45CCCN4C[C@H](F)C5)nc(c23)N2CCCOCC12]

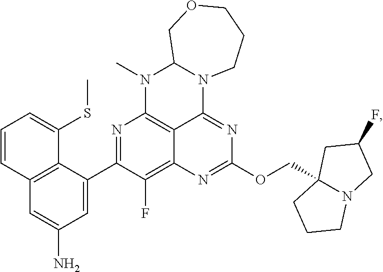 [SMILES: CSc1cccc2cc(N)cc(-c3nc4c5c(nc(OC[C@@]67CCCN6C[C@H](F)C7)nc5c3F)N3CCCOCC3N4C)c12]

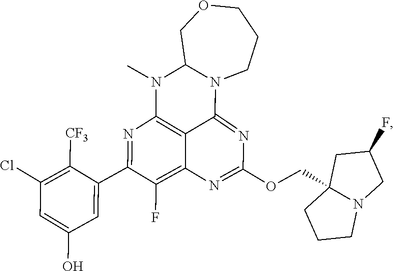 [SMILES: CN1c2nc(-c3cc(O)cc(Cl)c3C(F)(F)F)c(F)c3nc(OC[C@@]45CCCN4C[C@H](F)C5)nc(c23)N2CCCOCC12]

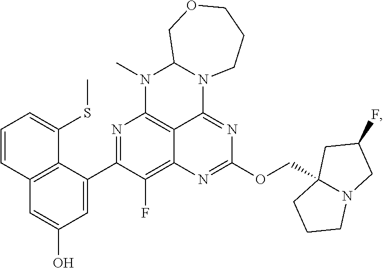 [SMILES: CSc1cccc2cc(O)cc(-c3nc4c5c(nc(OC[C@@]67CCCN6C[C@H](F)C7)nc5c3F)N3CCCOCC3N4C)c12]

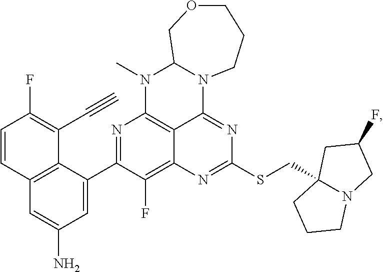 [SMILES: C#Cc1c(F)ccc2cc(N)cc(-c3nc4c5c(nc(SC[C@@]67CCCN6C[C@H](F)C7)nc5c3F)N3CCCOCC3N4C)c12]

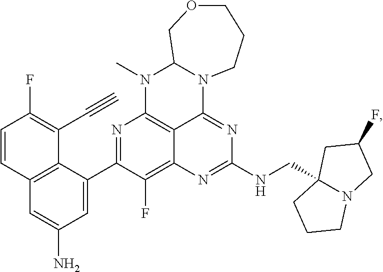 [SMILES: C#Cc1c(F)ccc2cc(N)cc(-c3nc4c5c(nc(NC[C@@]67CCCN6C[C@H](F)C7)nc5c3F)N3CCCOCC3N4C)c12]

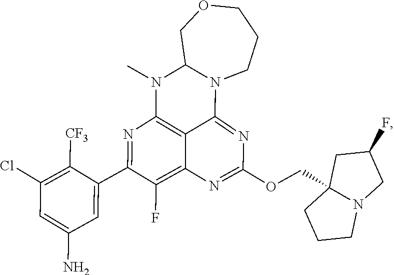 [SMILES: CN1c2nc(-c3cc(N)cc(Cl)c3C(F)(F)F)c(F)c3nc(OC[C@@]45CCCN4C[C@H](F)C5)nc(c23)N2CCCOCC12]

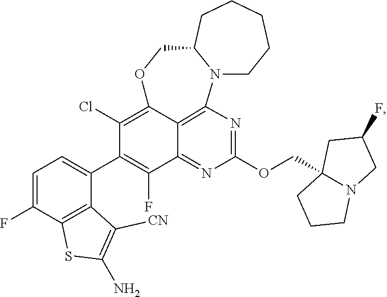 [SMILES: N#Cc1c(N)sc2c(F)ccc(-c3c(Cl)c4c5c(nc(OC[C@@]67CCCN6C[C@H](F)C7)nc5c3F)N3CCCCC[C@H]3CO4)c12]

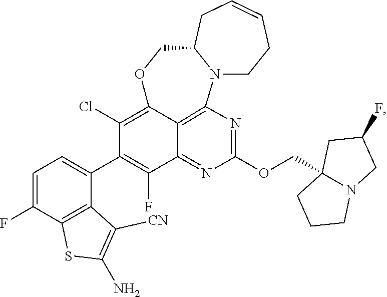 [SMILES: N#Cc1c(N)sc2c(F)ccc(-c3c(Cl)c4c5c(nc(OC[C@@]67CCCN6C[C@H](F)C7)nc5c3F)N3CCC=CC[C@H]3CO4)c12]

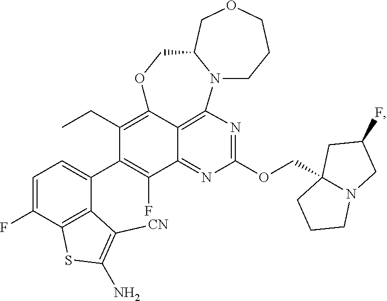 [SMILES: CCc1c(-c2ccc(F)c3sc(N)c(C#N)c23)c(F)c2nc(OC[C@@]34CCCN3C[C@H](F)C4)nc3c2c1OC[C@@H]1COCCCN31]

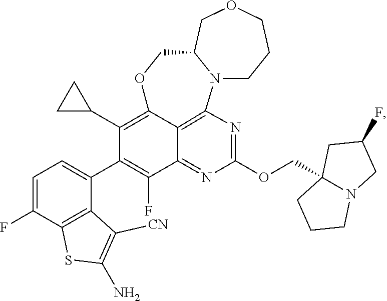 [SMILES: N#Cc1c(N)sc2c(F)ccc(-c3c(C4CC4)c4c5c(nc(OC[C@@]67CCCN6C[C@H](F)C7)nc5c3F)N3CCCOC[C@H]3CO4)c12]

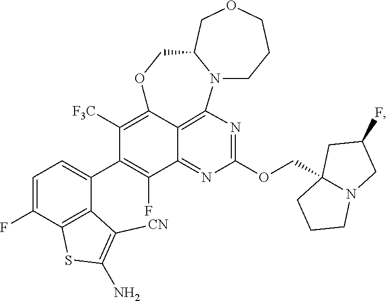 [SMILES: N#Cc1c(N)sc2c(F)ccc(-c3c(C(F)(F)F)c4c5c(nc(OC[C@@]67CCCN6C[C@H](F)C7)nc5c3F)N3CCCOC[C@H]3CO4)c12]

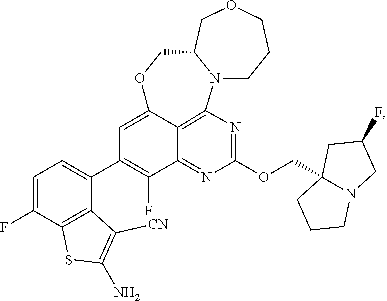 [SMILES: N#Cc1c(N)sc2c(F)ccc(-c3cc4c5c(nc(OC[C@@]67CCCN6C[C@H](F)C7)nc5c3F)N3CCCOC[C@H]3CO4)c12]

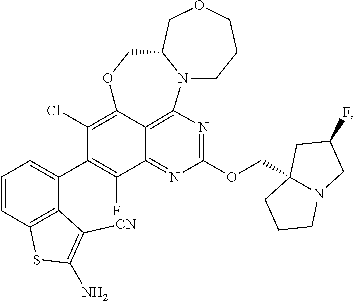 [SMILES: N#Cc1c(N)sc2cccc(-c3c(Cl)c4c5c(nc(OC[C@@]67CCCN6C[C@H](F)C7)nc5c3F)N3CCCOC[C@H]3CO4)c12]

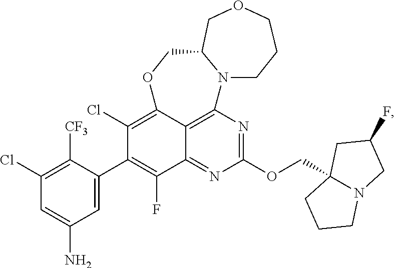 [SMILES: Nc1cc(Cl)c(C(F)(F)F)c(-c2c(Cl)c3c4c(nc(OC[C@@]56CCCN5C[C@H](F)C6)nc4c2F)N2CCCOC[C@H]2CO3)c1]

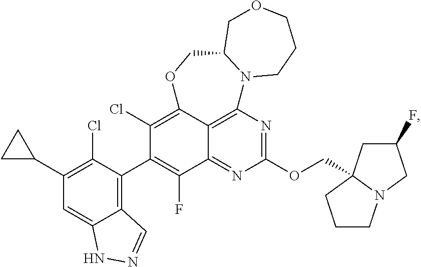 [SMILES: Fc1c(-c2c(Cl)c(C3CC3)cc3[nH]ncc23)c(Cl)c2c3c(nc(OC[C@@]45CCCN4C[C@H](F)C5)nc13)N1CCCOC[C@H]1CO2]

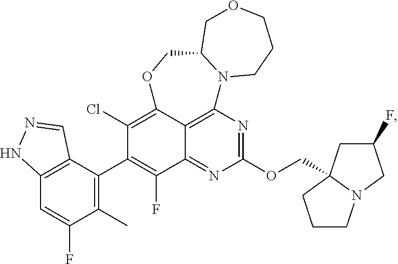 [SMILES: Cc1c(F)cc2[nH]ncc2c1-c1c(Cl)c2c3c(nc(OC[C@@]45CCCN4C[C@H](F)C5)nc3c1F)N1CCCOC[C@H]1CO2]

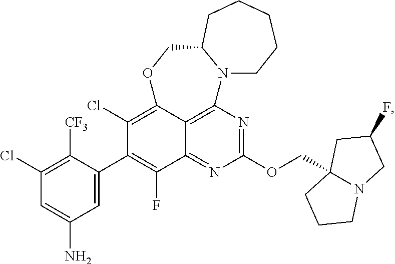 [SMILES: Nc1cc(Cl)c(C(F)(F)F)c(-c2c(Cl)c3c4c(nc(OC[C@@]56CCCN5C[C@H](F)C6)nc4c2F)N2CCCCC[C@H]2CO3)c1]

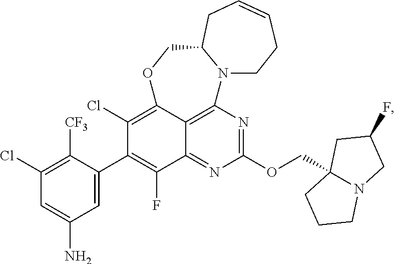 [SMILES: Nc1cc(Cl)c(C(F)(F)F)c(-c2c(Cl)c3c4c(nc(OC[C@@]56CCCN5C[C@H](F)C6)nc4c2F)N2CCC=CC[C@H]2CO3)c1]

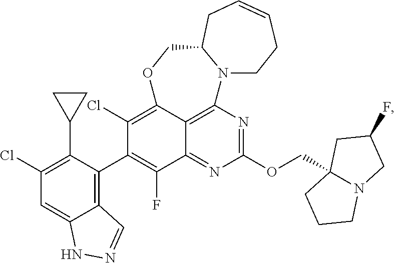 [SMILES: Fc1c(-c2c(C3CC3)c(Cl)cc3[nH]ncc23)c(Cl)c2c3c(nc(OC[C@@]45CCCN4C[C@H](F)C5)nc13)N1CCC=CC[C@H]1CO2]

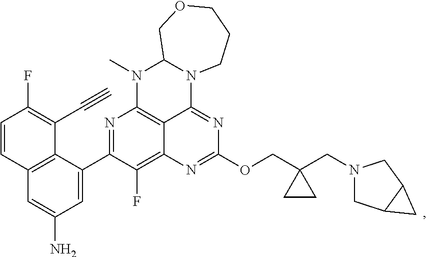 [SMILES: C#Cc1c(F)ccc2cc(N)cc(-c3nc4c5c(nc(OCC6(CN7CC8CC8C7)CC6)nc5c3F)N3CCCOCC3N4C)c12]

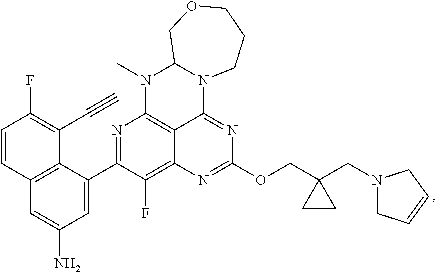 [SMILES: C#Cc1c(F)ccc2cc(N)cc(-c3nc4c5c(nc(OCC6(CN7CC=CC7)CC6)nc5c3F)N3CCCOCC3N4C)c12]

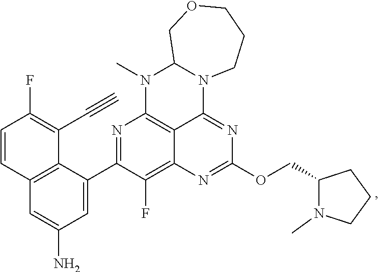 [SMILES: C#Cc1c(F)ccc2cc(N)cc(-c3nc4c5c(nc(OC[C@@H]6CCCN6C)nc5c3F)N3CCCOCC3N4C)c12]

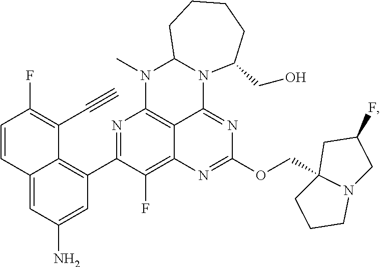 [SMILES: C#Cc1c(F)ccc2cc(N)cc(-c3nc4c5c(nc(OC[C@@]67CCCN6C[C@H](F)C7)nc5c3F)N3C(CCCC[C@@H]3CO)N4C)c12]

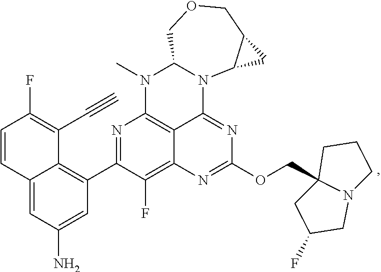 [SMILES: C#Cc1c(F)ccc2cc(N)cc(-c3nc4c5c(nc(OC[C@@]67CCCN6C[C@H](F)C7)nc5c3F)N3[C@H]5C[C@H]5COC[C@H]3N4C)c12]

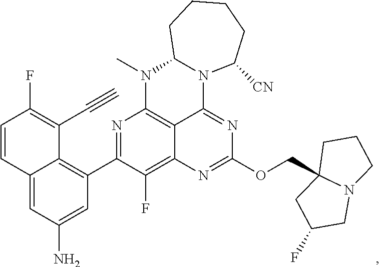 [SMILES: C#Cc1c(F)ccc2cc(N)cc(-c3nc4c5c(nc(OC[C@@]67CCCN6C[C@H](F)C7)nc5c3F)N3[C@@H](C#N)CCCC[C@H]3N4C)c12]

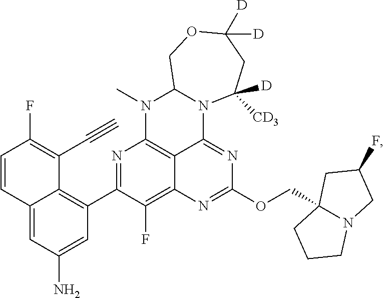 [SMILES: [2H]C1([2H])C[C@]([2H])(C([2H])([2H])[2H])N2c3nc(OC[C@@]45CCCN4C[C@H](F)C5)nc4c(F)c(-c5cc(N)cc6ccc(F)c(C#C)c56)nc(c34)N(C)C2CO1]

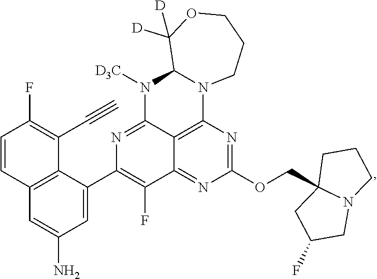 [SMILES: [2H]C1([2H])OCCCN2c3nc(OC[C@@]45CCCN4C[C@H](F)C5)nc4c(F)c(-c5cc(N)cc6ccc(F)c(C#C)c56)nc(c34)N(C([2H])([2H])[2H])[C@H]21 ; ","]

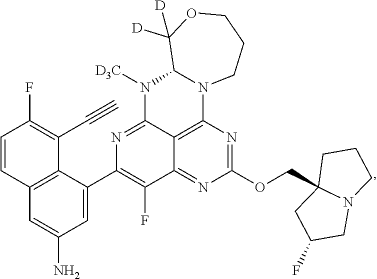 [SMILES: [2H]C1([2H])OCCCN2c3nc(OC[C@@]45CCCN4C[C@H](F)C5)nc4c(F)c(-c5cc(N)cc6ccc(F)c(C#C)c56)nc(c34)N(C([2H])([2H])[2H])[C@@H]21 ; ","]

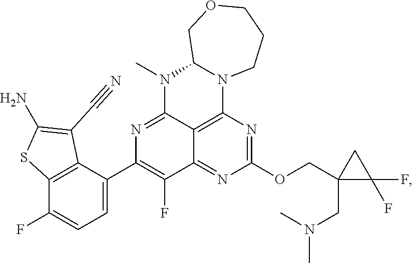 [SMILES: CN(C)CC1(COc2nc3c4c(nc(-c5ccc(F)c6sc(N)c(C#N)c56)c(F)c4n2)N(C)[C@@H]2COCCCN32)CC1(F)F]

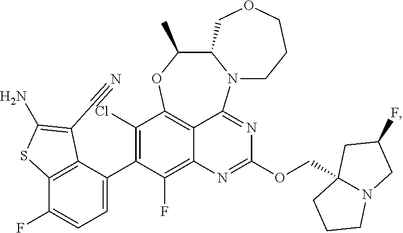 [SMILES: C[C@@H]1Oc2c(Cl)c(-c3ccc(F)c4sc(N)c(C#N)c34)c(F)c3nc(OC[C@@]45CCCN4C[C@H](F)C5)nc(c23)N2CCCOC[C@@H]12]

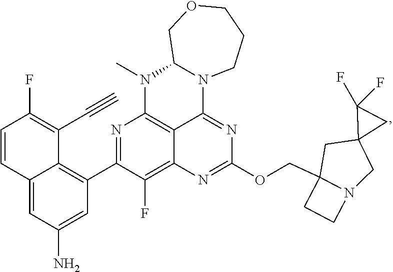 [SMILES: C#Cc1c(F)ccc2cc(N)cc(-c3nc4c5c(nc(OCC67CCN6CC6(C7)CC6(F)F)nc5c3F)N3CCCOC[C@H]3N4C)c12 ; ","]

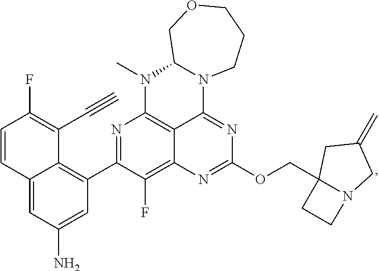 [SMILES: C#Cc1c(F)ccc2cc(N)cc(-c3nc4c5c(nc(OCC67CCN6CC(=C)C7)nc5c3F)N3CCCOC[C@H]3N4C)c12 ; ","]

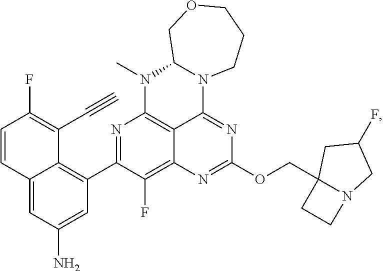 [SMILES: C#Cc1c(F)ccc2cc(N)cc(-c3nc4c5c(nc(OCC67CCN6CC(F)C7)nc5c3F)N3CCCOC[C@H]3N4C)c12]

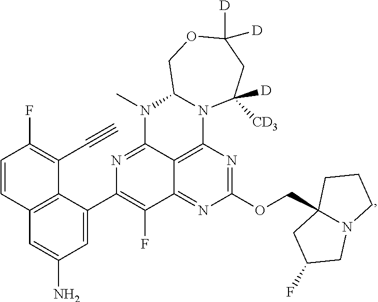 [SMILES: [2H]C1([2H])C[C@]([2H])(C([2H])([2H])[2H])N2c3nc(OC[C@@]45CCCN4C[C@H](F)C5)nc4c(F)c(-c5cc(N)cc6ccc(F)c(C#C)c56)nc(c34)N(C)[C@@H]2CO1 ; ","]

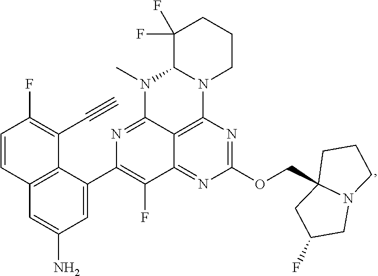 [SMILES: C#Cc1c(F)ccc2cc(N)cc(-c3nc4c5c(nc(OC[C@@]67CCCN6C[C@H](F)C7)nc5c3F)N3CCCC(F)(F)[C@H]3N4C)c12 ; ","]

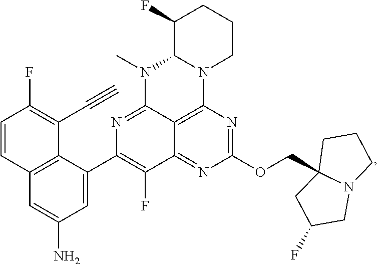 [SMILES: C#Cc1c(F)ccc2cc(N)cc(-c3nc4c5c(nc(OC[C@@]67CCCN6C[C@H](F)C7)nc5c3F)N3CCC[C@H](F)[C@H]3N4C)c12 ; ","]

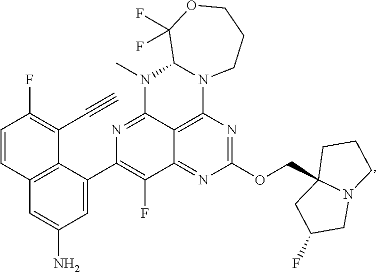 [SMILES: C#Cc1c(F)ccc2cc(N)cc(-c3nc4c5c(nc(OC[C@@]67CCCN6C[C@H](F)C7)nc5c3F)N3CCCOC(F)(F)[C@H]3N4C)c12 ; ","]

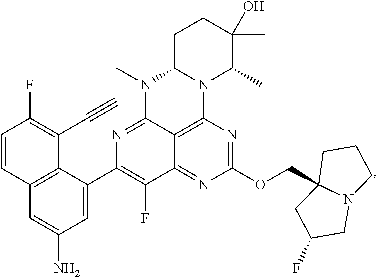 [SMILES: C#Cc1c(F)ccc2cc(N)cc(-c3nc4c5c(nc(OC[C@@]67CCCN6C[C@H](F)C7)nc5c3F)N3[C@@H](C)C(C)(O)CC[C@H]3N4C)c12 ; ","]

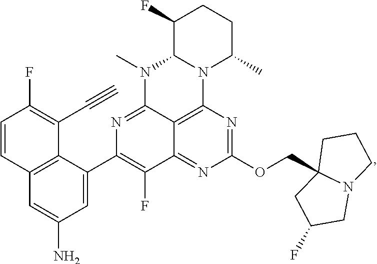 [SMILES: C#Cc1c(F)ccc2cc(N)cc(-c3nc4c5c(nc(OC[C@@]67CCCN6C[C@H](F)C7)nc5c3F)N3[C@@H](C)CC[C@H](F)[C@H]3N4C)c12 ; ","]

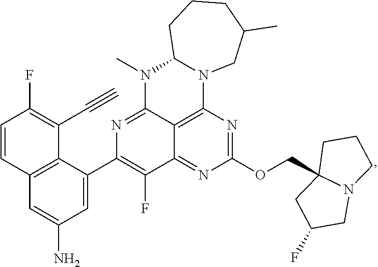 [SMILES: C#Cc1c(F)ccc2cc(N)cc(-c3nc4c5c(nc(OC[C@@]67CCCN6C[C@H](F)C7)nc5c3F)N3CC(C)CCC[C@H]3N4C)c12 ; ","]

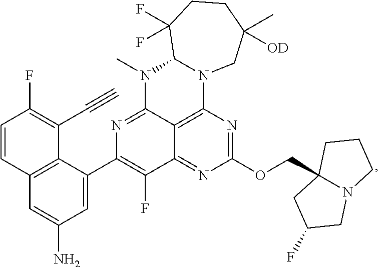

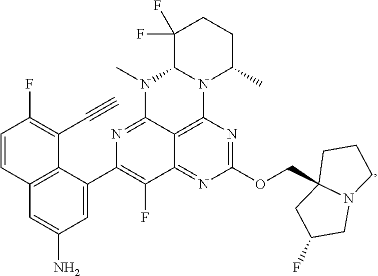 [SMILES: C#Cc1c(F)ccc2cc(N)cc(-c3nc4c5c(nc(OC[C@@]67CCCN6C[C@H](F)C7)nc5c3F)N3[C@@H](C)CCC(F)(F)[C@H]3N4C)c12 ; ","]

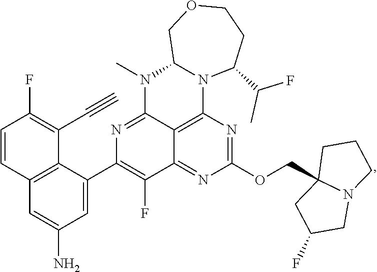 [SMILES: C#Cc1c(F)ccc2cc(N)cc(-c3nc4c5c(nc(OC[C@@]67CCCN6C[C@H](F)C7)nc5c3F)N3[C@@H](C(C)F)CCOC[C@H]3N4C)c12 ; ","]

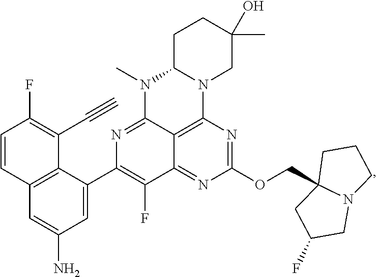 [SMILES: C#Cc1c(F)ccc2cc(N)cc(-c3nc4c5c(nc(OC[C@@]67CCCN6C[C@H](F)C7)nc5c3F)N3CC(C)(O)CC[C@H]3N4C)c12 ; ","]

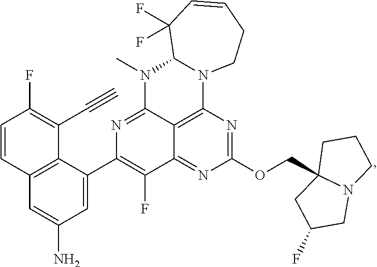 [SMILES: C#Cc1c(F)ccc2cc(N)cc(-c3nc4c5c(nc(OC[C@@]67CCCN6C[C@H](F)C7)nc5c3F)N3CCC=CC(F)(F)[C@H]3N4C)c12 ; ","]

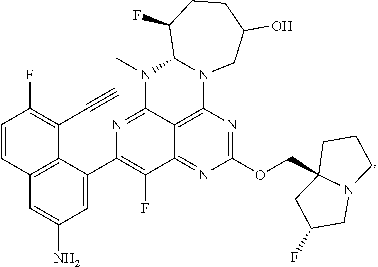 [SMILES: C#Cc1c(F)ccc2cc(N)cc(-c3nc4c5c(nc(OC[C@@]67CCCN6C[C@H](F)C7)nc5c3F)N3CC(O)CC[C@H](F)[C@H]3N4C)c12 ; ","]

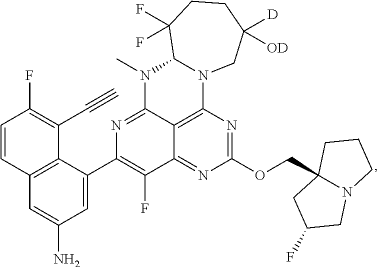

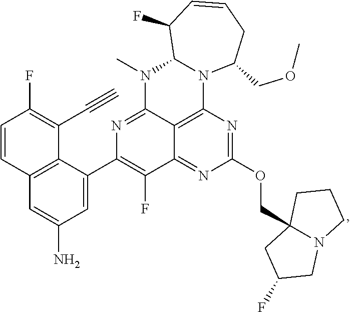 [SMILES: C#Cc1c(F)ccc2cc(N)cc(-c3nc4c5c(nc(OC[C@@]67CCCN6C[C@H](F)C7)nc5c3F)N3[C@@H](COC)CC=C[C@H](F)[C@H]3N4C)c12 ; ","]

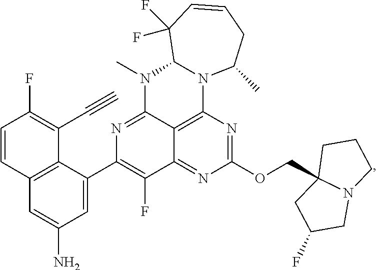 [SMILES: C#Cc1c(F)ccc2cc(N)cc(-c3nc4c5c(nc(OC[C@@]67CCCN6C[C@H](F)C7)nc5c3F)N3[C@@H](C)CC=CC(F)(F)[C@H]3N4C)c12 ; ","]

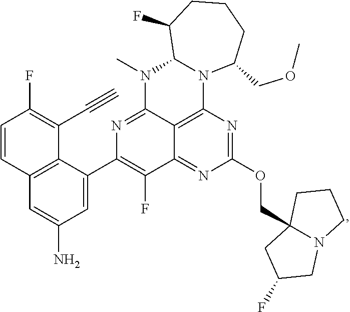 [SMILES: C#Cc1c(F)ccc2cc(N)cc(-c3nc4c5c(nc(OC[C@@]67CCCN6C[C@H](F)C7)nc5c3F)N3[C@@H](COC)CCC[C@H](F)[C@H]3N4C)c12 ; ","]

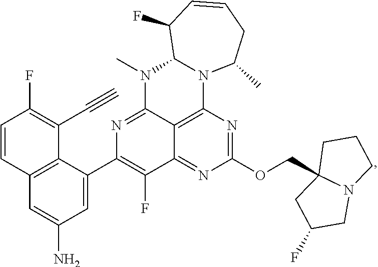 [SMILES: C#Cc1c(F)ccc2cc(N)cc(-c3nc4c5c(nc(OC[C@@]67CCCN6C[C@H](F)C7)nc5c3F)N3[C@@H](C)CC=C[C@H](F)[C@H]3N4C)c12 ; ","]

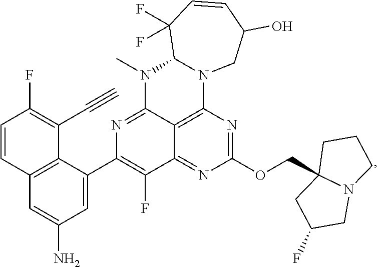 [SMILES: C#Cc1c(F)ccc2cc(N)cc(-c3nc4c5c(nc(OC[C@@]67CCCN6C[C@H](F)C7)nc5c3F)N3CC(O)C=CC(F)(F)[C@H]3N4C)c12 ; ","]

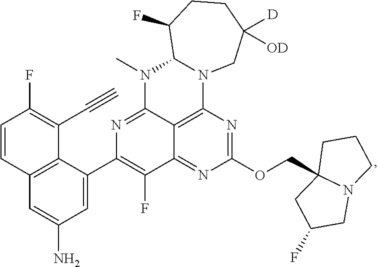

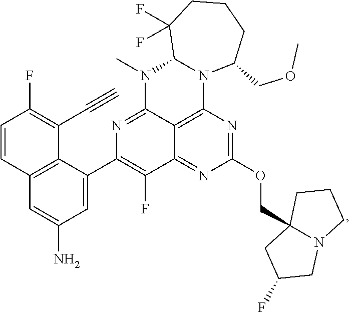 [SMILES: C#Cc1c(F)ccc2cc(N)cc(-c3nc4c5c(nc(OC[C@@]67CCCN6C[C@H](F)C7)nc5c3F)N3[C@@H](COC)CCCC(F)(F)[C@H]3N4C)c12 ; ","]

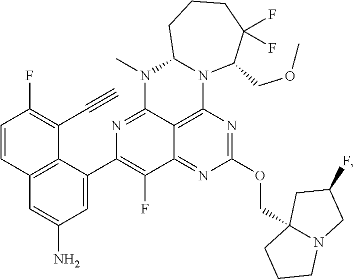 [SMILES: C#Cc1c(F)ccc2cc(N)cc(-c3nc4c5c(nc(OC[C@@]67CCCN6C[C@H](F)C7)nc5c3F)N3[C@@H](COC)C(F)(F)CCC[C@H]3N4C)c12]

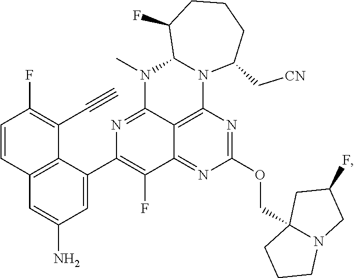 [SMILES: C#Cc1c(F)ccc2cc(N)cc(-c3nc4c5c(nc(OC[C@@]67CCCN6C[C@H](F)C7)nc5c3F)N3[C@@H](CC#N)CCC[C@H](F)[C@H]3N4C)c12]

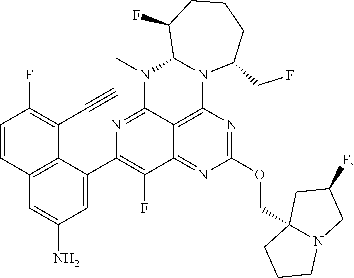 [SMILES: C#Cc1c(F)ccc2cc(N)cc(-c3nc4c5c(nc(OC[C@@]67CCCN6C[C@H](F)C7)nc5c3F)N3[C@@H](CF)CCC[C@H](F)[C@H]3N4C)c12]

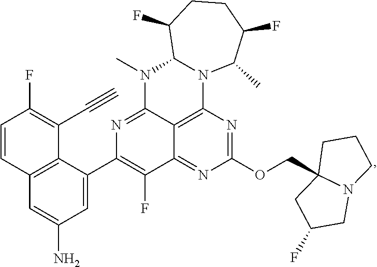 [SMILES: C#Cc1c(F)ccc2cc(N)cc(-c3nc4c5c(nc(OC[C@@]67CCCN6C[C@H](F)C7)nc5c3F)N3[C@@H](C)[C@H](F)CC[C@H](F)[C@H]3N4C)c12 ; ","]

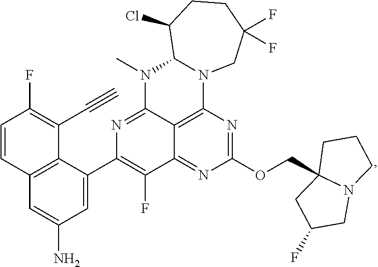 [SMILES: C#Cc1c(F)ccc2cc(N)cc(-c3nc4c5c(nc(OC[C@@]67CCCN6C[C@H](F)C7)nc5c3F)N3CC(F)(F)CC[C@H](Cl)[C@H]3N4C)c12 ; ","]

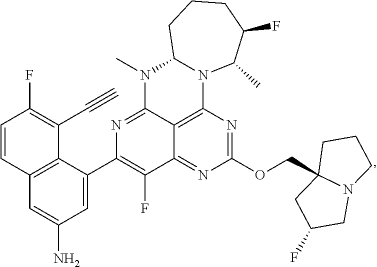 [SMILES: C#Cc1c(F)ccc2cc(N)cc(-c3nc4c5c(nc(OC[C@@]67CCCN6C[C@H](F)C7)nc5c3F)N3[C@@H](C)[C@H](F)CCC[C@H]3N4C)c12 ; ","]

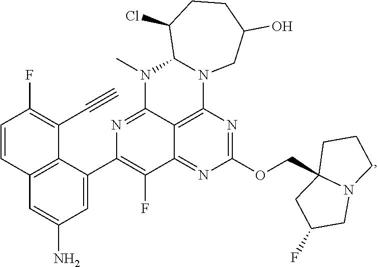 [SMILES: C#Cc1c(F)ccc2cc(N)cc(-c3nc4c5c(nc(OC[C@@]67CCCN6C[C@H](F)C7)nc5c3F)N3CC(O)CC[C@H](Cl)[C@H]3N4C)c12 ; ","]

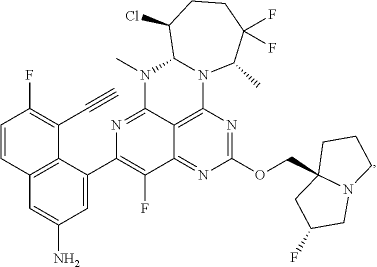 [SMILES: C#Cc1c(F)ccc2cc(N)cc(-c3nc4c5c(nc(OC[C@@]67CCCN6C[C@H](F)C7)nc5c3F)N3[C@@H](C)C(F)(F)CC[C@H](Cl)[C@H]3N4C)c12 ; ","]

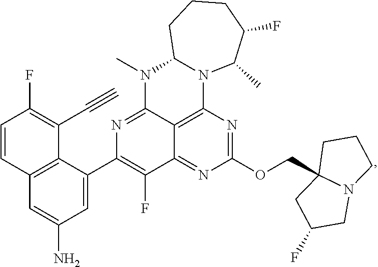 [SMILES: C#Cc1c(F)ccc2cc(N)cc(-c3nc4c5c(nc(OC[C@@]67CCCN6C[C@H](F)C7)nc5c3F)N3[C@@H](C)[C@@H](F)CCC[C@H]3N4C)c12 ; ","]

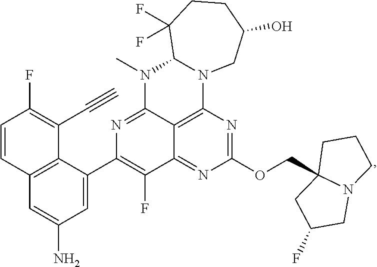 [SMILES: C#Cc1c(F)ccc2cc(N)cc(-c3nc4c5c(nc(OC[C@@]67CCCN6C[C@H](F)C7)nc5c3F)N3C[C@@H](O)CCC(F)(F)[C@H]3N4C)c12 ; ","]

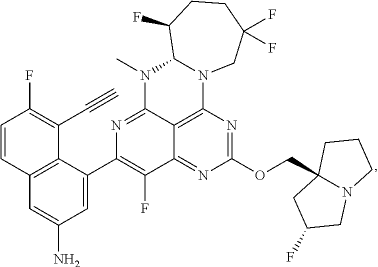 [SMILES: C#Cc1c(F)ccc2cc(N)cc(-c3nc4c5c(nc(OC[C@@]67CCCN6C[C@H](F)C7)nc5c3F)N3CC(F)(F)CC[C@H](F)[C@H]3N4C)c12 ; ","]

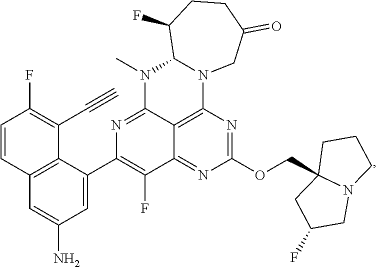 [SMILES: C#Cc1c(F)ccc2cc(N)cc(-c3nc4c5c(nc(OC[C@@]67CCCN6C[C@H](F)C7)nc5c3F)N3CC(=O)CC[C@H](F)[C@H]3N4C)c12 ; ","]

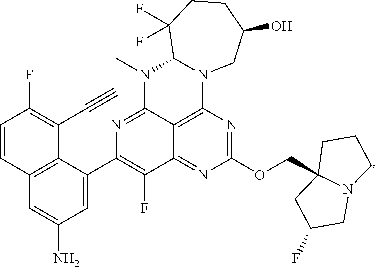 [SMILES: C#Cc1c(F)ccc2cc(N)cc(-c3nc4c5c(nc(OC[C@@]67CCCN6C[C@H](F)C7)nc5c3F)N3C[C@H](O)CCC(F)(F)[C@H]3N4C)c12 ; ","]

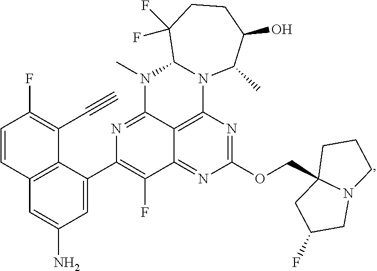 [SMILES: C#Cc1c(F)ccc2cc(N)cc(-c3nc4c5c(nc(OC[C@@]67CCCN6C[C@H](F)C7)nc5c3F)N3[C@@H](C)[C@H](O)CCC(F)(F)[C@H]3N4C)c12 ; ","]

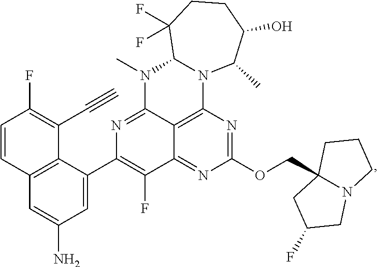 [SMILES: C#Cc1c(F)ccc2cc(N)cc(-c3nc4c5c(nc(OC[C@@]67CCCN6C[C@H](F)C7)nc5c3F)N3[C@@H](C)[C@@H](O)CCC(F)(F)[C@H]3N4C)c12 ; ","]

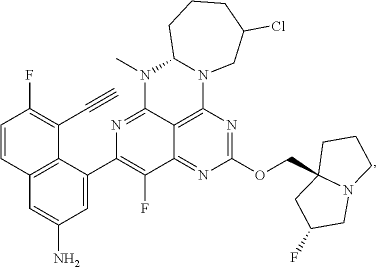 [SMILES: C#Cc1c(F)ccc2cc(N)cc(-c3nc4c5c(nc(OC[C@@]67CCCN6C[C@H](F)C7)nc5c3F)N3CC(Cl)CCC[C@H]3N4C)c12 ; ","]

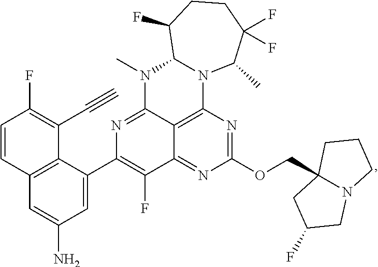 [SMILES: C#Cc1c(F)ccc2cc(N)cc(-c3nc4c5c(nc(OC[C@@]67CCCN6C[C@H](F)C7)nc5c3F)N3[C@@H](C)C(F)(F)CC[C@H](F)[C@H]3N4C)c12 ; ","]

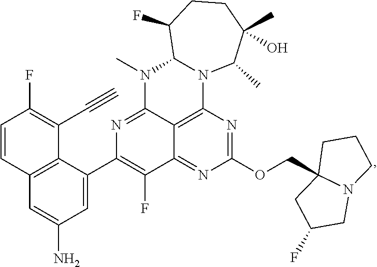 [SMILES: C#Cc1c(F)ccc2cc(N)cc(-c3nc4c5c(nc(OC[C@@]67CCCN6C[C@H](F)C7)nc5c3F)N3[C@@H](C)[C@@](C)(O)CC[C@H](F)[C@H]3N4C)c12 ; ","]

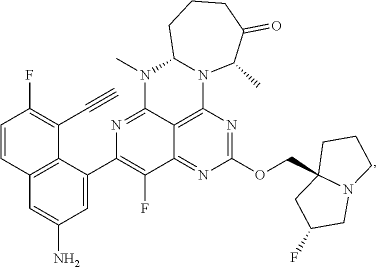 [SMILES: C#Cc1c(F)ccc2cc(N)cc(-c3nc4c5c(nc(OC[C@@]67CCCN6C[C@H](F)C7)nc5c3F)N3[C@@H](C)C(=O)CCC[C@H]3N4C)c12 ; ","]

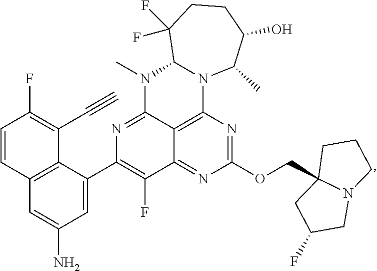 [SMILES: C#Cc1c(F)ccc2cc(N)cc(-c3nc4c5c(nc(OC[C@@]67CCCN6C[C@H](F)C7)nc5c3F)N3[C@@H](C)[C@@H](O)CCC(F)(F)[C@H]3N4C)c12 ; ","]

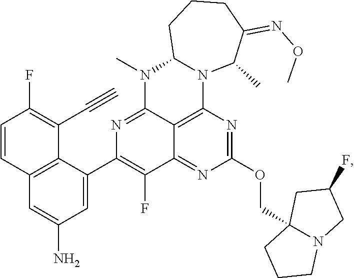 [SMILES: C#Cc1c(F)ccc2cc(N)cc(-c3nc4c5c(nc(OC[C@@]67CCCN6C[C@H](F)C7)nc5c3F)N3[C@@H](C)/C(=N\OC)CCC[C@H]3N4C)c12]

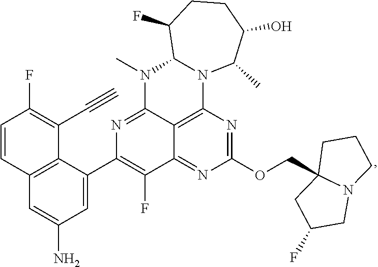 [SMILES: C#Cc1c(F)ccc2cc(N)cc(-c3nc4c5c(nc(OC[C@@]67CCCN6C[C@H](F)C7)nc5c3F)N3[C@@H](C)[C@@H](O)CC[C@H](F)[C@H]3N4C)c12 ; ","]

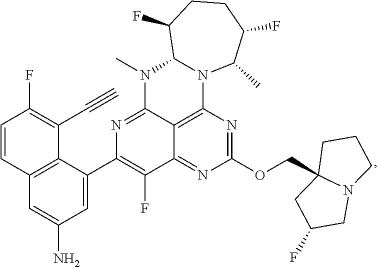 [SMILES: C#Cc1c(F)ccc2cc(N)cc(-c3nc4c5c(nc(OC[C@@]67CCCN6C[C@H](F)C7)nc5c3F)N3[C@@H](C)[C@@H](F)CC[C@H](F)[C@H]3N4C)c12 ; ","]

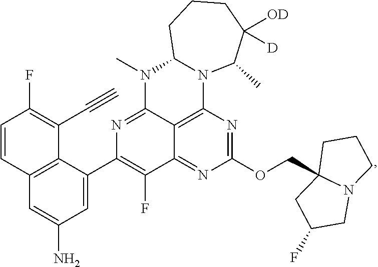

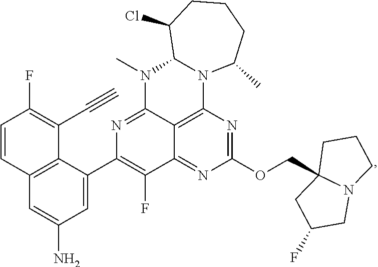 [SMILES: C#Cc1c(F)ccc2cc(N)cc(-c3nc4c5c(nc(OC[C@@]67CCCN6C[C@H](F)C7)nc5c3F)N3[C@@H](C)CCC[C@H](Cl)[C@H]3N4C)c12 ; ","]

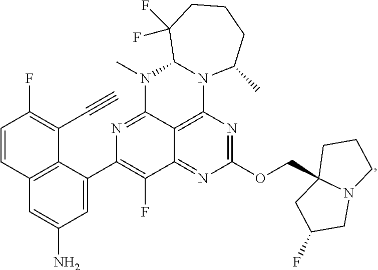 [SMILES: C#Cc1c(F)ccc2cc(N)cc(-c3nc4c5c(nc(OC[C@@]67CCCN6C[C@H](F)C7)nc5c3F)N3[C@@H](C)CCCC(F)(F)[C@H]3N4C)c12 ; ","]

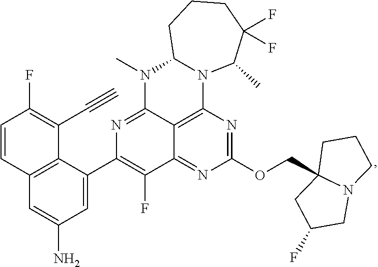 [SMILES: C#Cc1c(F)ccc2cc(N)cc(-c3nc4c5c(nc(OC[C@@]67CCCN6C[C@H](F)C7)nc5c3F)N3[C@@H](C)C(F)(F)CCC[C@H]3N4C)c12 ; ","]

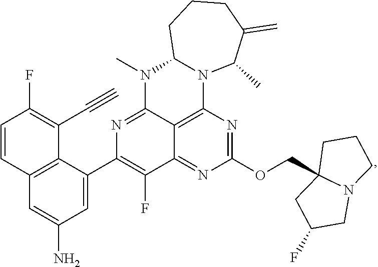 [SMILES: C#Cc1c(F)ccc2cc(N)cc(-c3nc4c5c(nc(OC[C@@]67CCCN6C[C@H](F)C7)nc5c3F)N3[C@@H](C)C(=C)CCC[C@H]3N4C)c12 ; ","]

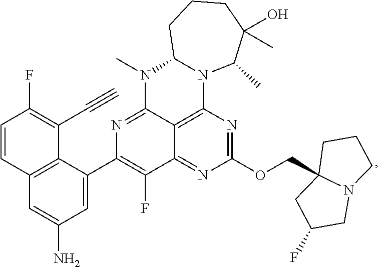 [SMILES: C#Cc1c(F)ccc2cc(N)cc(-c3nc4c5c(nc(OC[C@@]67CCCN6C[C@H](F)C7)nc5c3F)N3[C@@H](C)C(C)(O)CCC[C@H]3N4C)c12 ; ","]

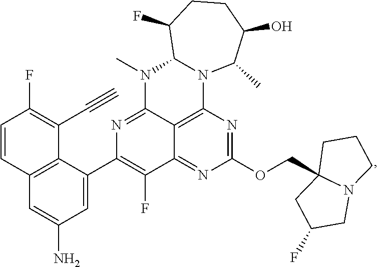 [SMILES: C#Cc1c(F)ccc2cc(N)cc(-c3nc4c5c(nc(OC[C@@]67CCCN6C[C@H](F)C7)nc5c3F)N3[C@@H](C)[C@H](O)CC[C@H](F)[C@H]3N4C)c12 ; ","]

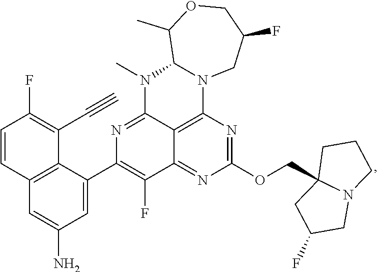 [SMILES: C#Cc1c(F)ccc2cc(N)cc(-c3nc4c5c(nc(OC[C@@]67CCCN6C[C@H](F)C7)nc5c3F)N3C[C@H](F)COC(C)[C@H]3N4C)c12 ; ","]

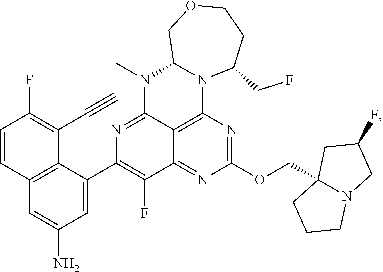 [SMILES: C#Cc1c(F)ccc2cc(N)cc(-c3nc4c5c(nc(OC[C@@]67CCCN6C[C@H](F)C7)nc5c3F)N3[C@@H](CF)CCOC[C@H]3N4C)c12]

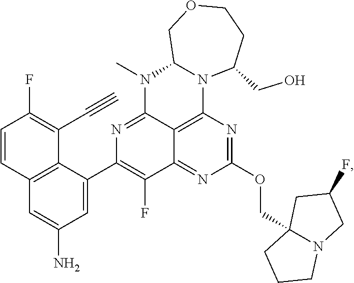 [SMILES: C#Cc1c(F)ccc2cc(N)cc(-c3nc4c5c(nc(OC[C@@]67CCCN6C[C@H](F)C7)nc5c3F)N3[C@@H](CO)CCOC[C@H]3N4C)c12]

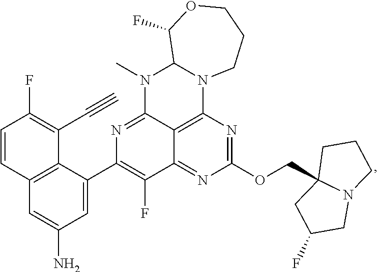 [SMILES: C#Cc1c(F)ccc2cc(N)cc(-c3nc4c5c(nc(OC[C@@]67CCCN6C[C@H](F)C7)nc5c3F)N3CCCO[C@@H](F)C3N4C)c12 ; ","]

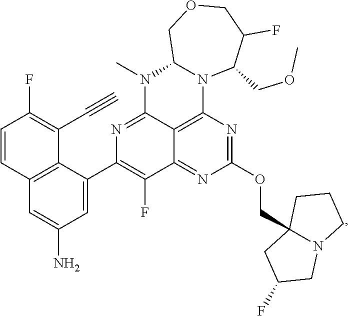 [SMILES: C#Cc1c(F)ccc2cc(N)cc(-c3nc4c5c(nc(OC[C@@]67CCCN6C[C@H](F)C7)nc5c3F)N3[C@@H](COC)C(F)COC[C@H]3N4C)c12 ; ","]

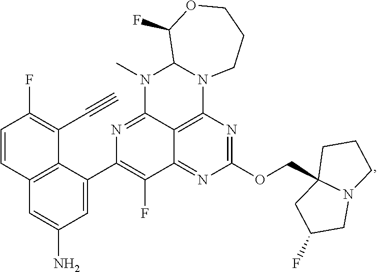 [SMILES: C#Cc1c(F)ccc2cc(N)cc(-c3nc4c5c(nc(OC[C@@]67CCCN6C[C@H](F)C7)nc5c3F)N3CCCO[C@H](F)C3N4C)c12 ; ","]

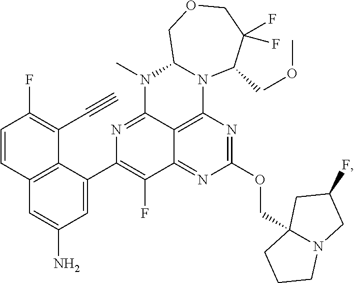

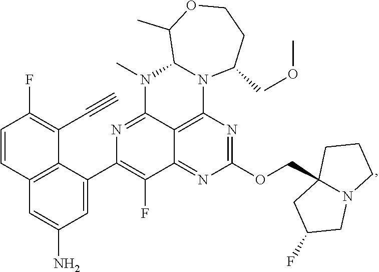

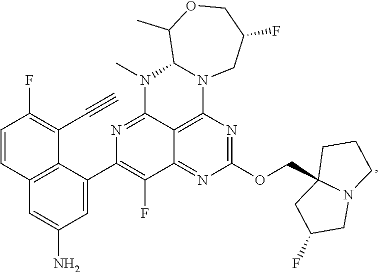 [SMILES: C#Cc1c(F)ccc2cc(N)cc(-c3nc4c5c(nc(OC[C@@]67CCCN6C[C@H](F)C7)nc5c3F)N3C[C@@H](F)COC(C)[C@H]3N4C)c12 ; ","]

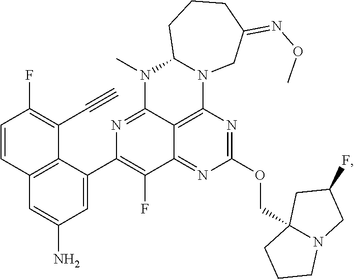 [SMILES: C#Cc1c(F)ccc2cc(N)cc(-c3nc4c5c(nc(OC[C@@]67CCCN6C[C@H](F)C7)nc5c3F)N3C/C(=N\OC)CCC[C@H]3N4C)c12]

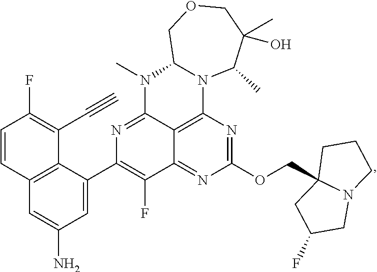 [SMILES: C#Cc1c(F)ccc2cc(N)cc(-c3nc4c5c(nc(OC[C@@]67CCCN6C[C@H](F)C7)nc5c3F)N3[C@@H](C)C(C)(O)COC[C@H]3N4C)c12 ; ","]

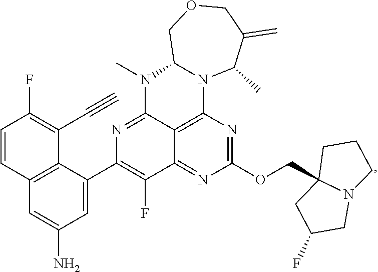 [SMILES: C#Cc1c(F)ccc2cc(N)cc(-c3nc4c5c(nc(OC[C@@]67CCCN6C[C@H](F)C7)nc5c3F)N3[C@@H](C)C(=C)COC[C@H]3N4C)c12 ; ","]

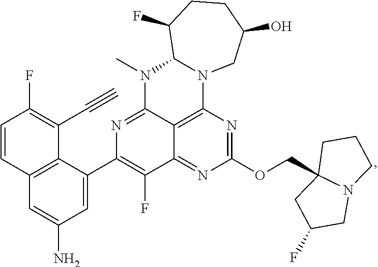 [SMILES: C#Cc1c(F)ccc2cc(N)cc(-c3nc4c5c(nc(OC[C@@]67CCCN6C[C@H](F)C7)nc5c3F)N3C[C@H](O)CC[C@H](F)[C@H]3N4C)c12 ; ","]

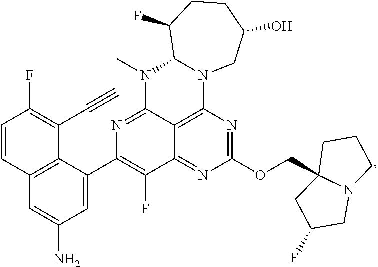 [SMILES: C#Cc1c(F)ccc2cc(N)cc(-c3nc4c5c(nc(OC[C@@]67CCCN6C[C@H](F)C7)nc5c3F)N3C[C@@H](O)CC[C@H](F)[C@H]3N4C)c12 ; ","]

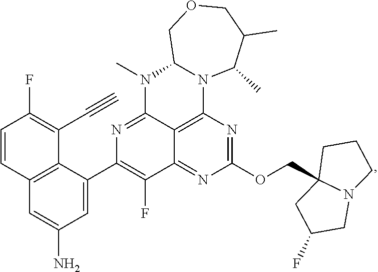 [SMILES: C#Cc1c(F)ccc2cc(N)cc(-c3nc4c5c(nc(OC[C@@]67CCCN6C[C@H](F)C7)nc5c3F)N3[C@@H](C)C(C)COC[C@H]3N4C)c12 ; ","]

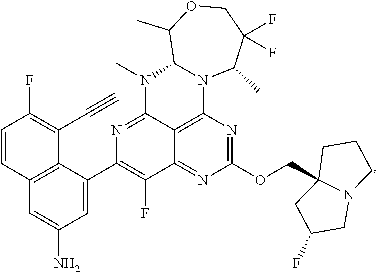 [SMILES: C#Cc1c(F)ccc2cc(N)cc(-c3nc4c5c(nc(OC[C@@]67CCCN6C[C@H](F)C7)nc5c3F)N3[C@@H](C)C(F)(F)COC(C)[C@H]3N4C)c12 ; ","]

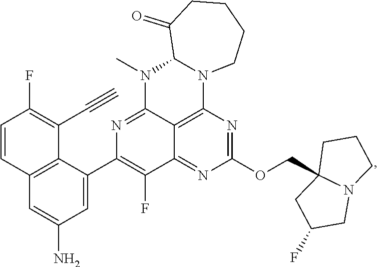 [SMILES: C#Cc1c(F)ccc2cc(N)cc(-c3nc4c5c(nc(OC[C@@]67CCCN6C[C@H](F)C7)nc5c3F)N3CCCCC(=O)[C@H]3N4C)c12 ; ","]

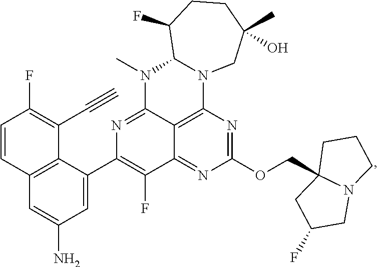 [SMILES: C#Cc1c(F)ccc2cc(N)cc(-c3nc4c5c(nc(OC[C@@]67CCCN6C[C@H](F)C7)nc5c3F)N3C[C@@](C)(O)CC[C@H](F)[C@H]3N4C)c12 ; ","]

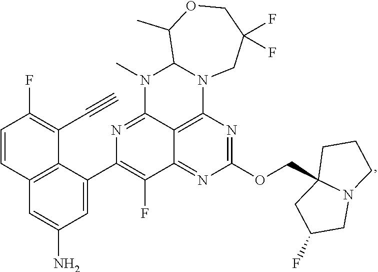 [SMILES: C#Cc1c(F)ccc2cc(N)cc(-c3nc4c5c(nc(OC[C@@]67CCCN6C[C@H](F)C7)nc5c3F)N3CC(F)(F)COC(C)C3N4C)c12 ; ","]

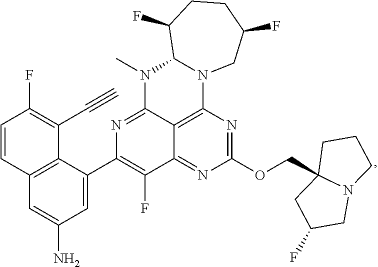 [SMILES: C#Cc1c(F)ccc2cc(N)cc(-c3nc4c5c(nc(OC[C@@]67CCCN6C[C@H](F)C7)nc5c3F)N3C[C@H](F)CC[C@H](F)[C@H]3N4C)c12 ; ","]

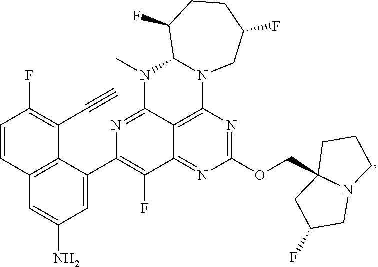 [SMILES: C#Cc1c(F)ccc2cc(N)cc(-c3nc4c5c(nc(OC[C@@]67CCCN6C[C@H](F)C7)nc5c3F)N3C[C@@H](F)CC[C@H](F)[C@H]3N4C)c12 ; ","]

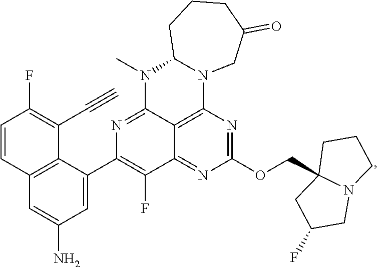 [SMILES: C#Cc1c(F)ccc2cc(N)cc(-c3nc4c5c(nc(OC[C@@]67CCCN6C[C@H](F)C7)nc5c3F)N3CC(=O)CCC[C@H]3N4C)c12 ; ","]

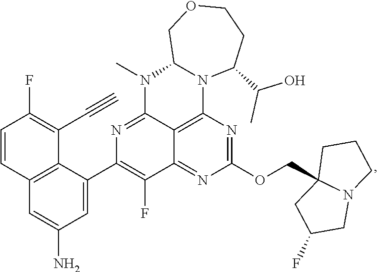 [SMILES: C#Cc1c(F)ccc2cc(N)cc(-c3nc4c5c(nc(OC[C@@]67CCCN6C[C@H](F)C7)nc5c3F)N3[C@@H](C(C)O)CCOC[C@H]3N4C)c12 ; ","]

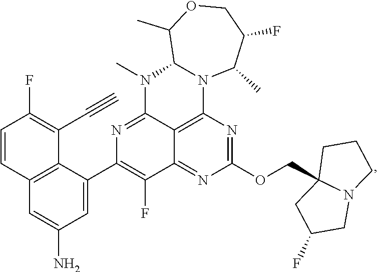 [SMILES: C#Cc1c(F)ccc2cc(N)cc(-c3nc4c5c(nc(OC[C@@]67CCCN6C[C@H](F)C7)nc5c3F)N3[C@@H](C)[C@@H](F)COC(C)[C@H]3N4C)c12 ; ","]

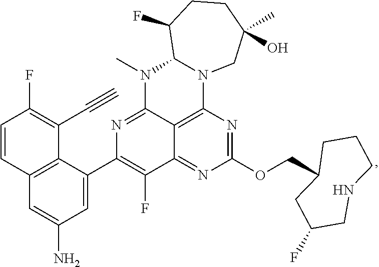

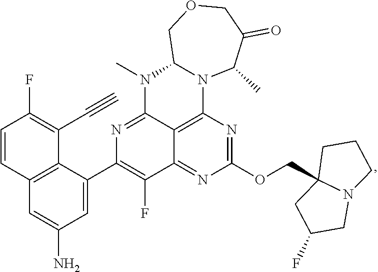 [SMILES: C#Cc1c(F)ccc2cc(N)cc(-c3nc4c5c(nc(OC[C@@]67CCCN6C[C@H](F)C7)nc5c3F)N3[C@@H](C)C(=O)COC[C@H]3N4C)c12 ; ","]

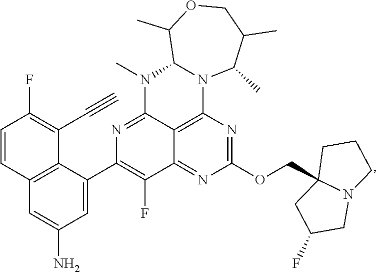 [SMILES: C#Cc1c(F)ccc2cc(N)cc(-c3nc4c5c(nc(OC[C@@]67CCCN6C[C@H](F)C7)nc5c3F)N3[C@@H](C)C(C)COC(C)[C@H]3N4C)c12 ; ","]

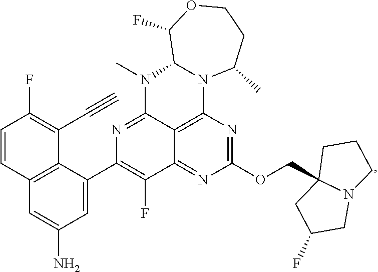 [SMILES: C#Cc1c(F)ccc2cc(N)cc(-c3nc4c5c(nc(OC[C@@]67CCCN6C[C@H](F)C7)nc5c3F)N3[C@@H](C)CCO[C@@H](F)[C@H]3N4C)c12 ; ","]

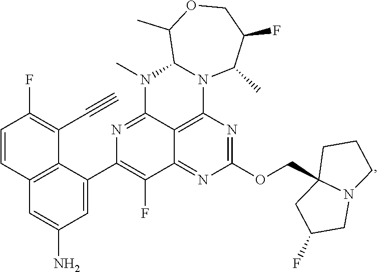 [SMILES: C#Cc1c(F)ccc2cc(N)cc(-c3nc4c5c(nc(OC[C@@]67CCCN6C[C@H](F)C7)nc5c3F)N3[C@@H](C)[C@H](F)COC(C)[C@H]3N4C)c12 ; ","]

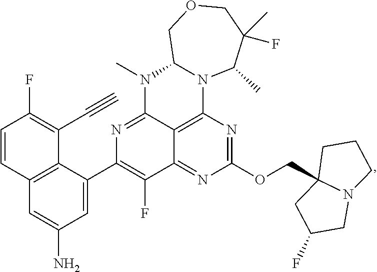 [SMILES: C#Cc1c(F)ccc2cc(N)cc(-c3nc4c5c(nc(OC[C@@]67CCCN6C[C@H](F)C7)nc5c3F)N3[C@@H](C)C(C)(F)COC[C@H]3N4C)c12 ; ","]

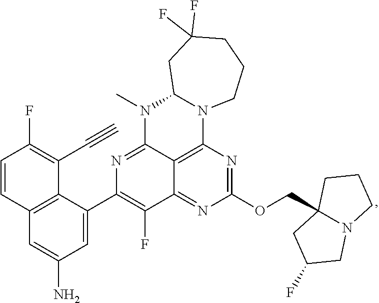 [SMILES: C#Cc1c(F)ccc2cc(N)cc(-c3nc4c5c(nc(OC[C@@]67CCCN6C[C@H](F)C7)nc5c3F)N3CCCC(F)(F)C[C@H]3N4C)c12 ; ","]

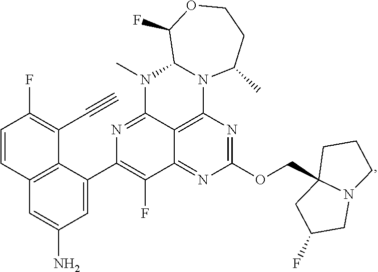 [SMILES: C#Cc1c(F)ccc2cc(N)cc(-c3nc4c5c(nc(OC[C@@]67CCCN6C[C@H](F)C7)nc5c3F)N3[C@@H](C)CCO[C@H](F)[C@H]3N4C)c12 ; ","]

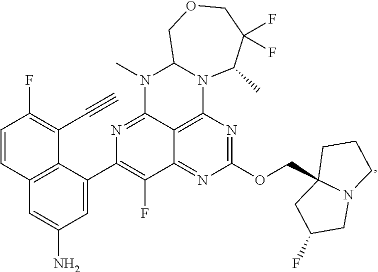 [SMILES: C#Cc1c(F)ccc2cc(N)cc(-c3nc4c5c(nc(OC[C@@]67CCCN6C[C@H](F)C7)nc5c3F)N3C(COCC(F)(F)[C@@H]3C)N4C)c12 ; ","]

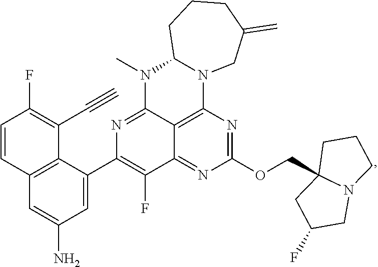 [SMILES: C#Cc1c(F)ccc2cc(N)cc(-c3nc4c5c(nc(OC[C@@]67CCCN6C[C@H](F)C7)nc5c3F)N3CC(=C)CCC[C@H]3N4C)c12 ; ","]

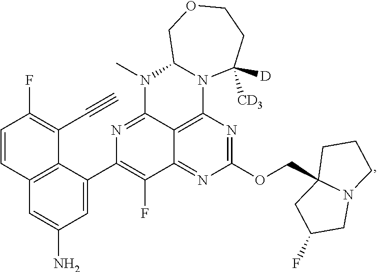 [SMILES: [2H]C([2H])([2H])[C@@]1([2H])CCOC[C@H]2N(C)c3nc(-c4cc(N)cc5ccc(F)c(C#C)c45)c(F)c4nc(OC[C@@]56CCCN5C[C@H](F)C6)nc(c34)N21 ; ","]

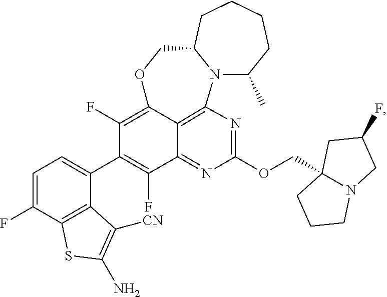 [SMILES: C[C@H]1CCCC[C@H]2COc3c(F)c(-c4ccc(F)c5sc(N)c(C#N)c45)c(F)c4nc(OC[C@@]56CCCN5C[C@H](F)C6)nc(c34)N21]

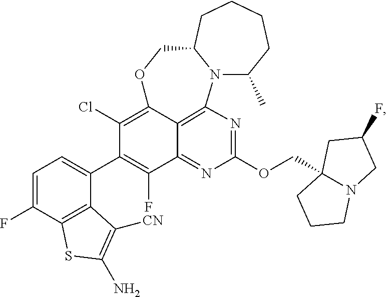 [SMILES: C[C@H]1CCCC[C@H]2COc3c(Cl)c(-c4ccc(F)c5sc(N)c(C#N)c45)c(F)c4nc(OC[C@@]56CCCN5C[C@H](F)C6)nc(c34)N21]

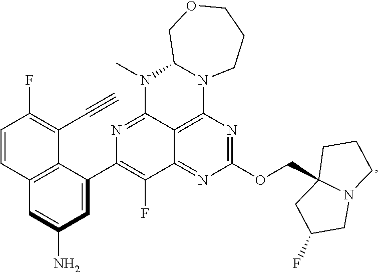

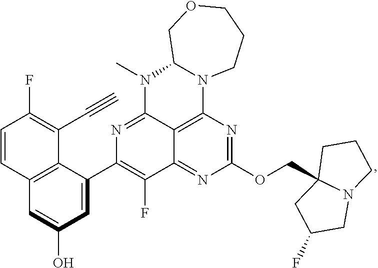

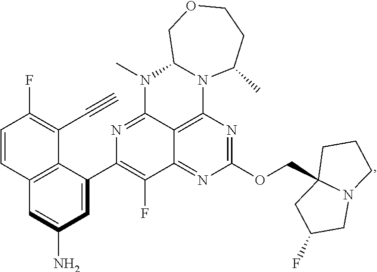

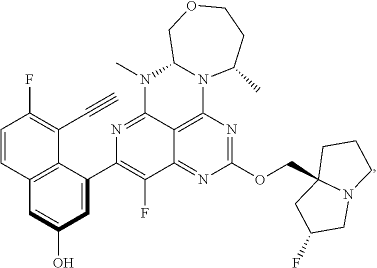

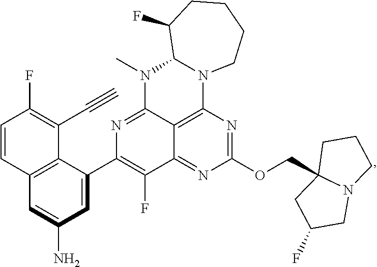

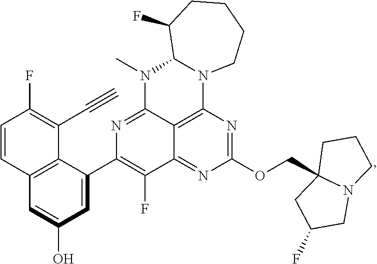

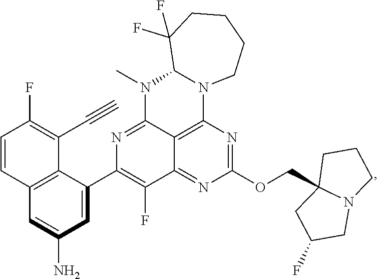

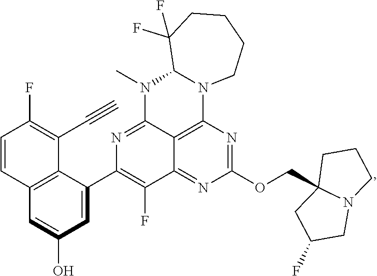

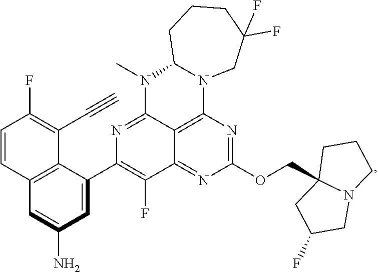

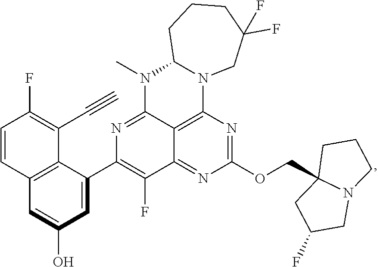

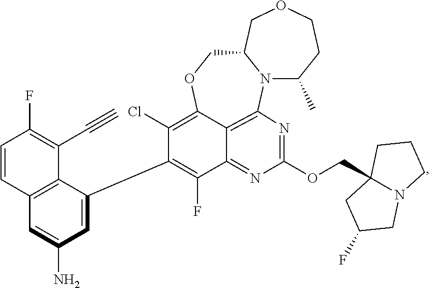

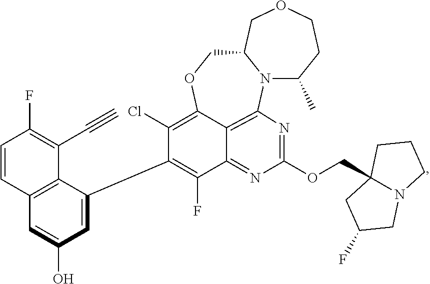

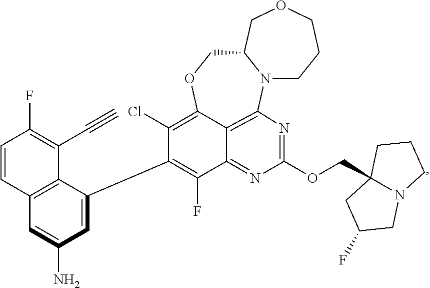

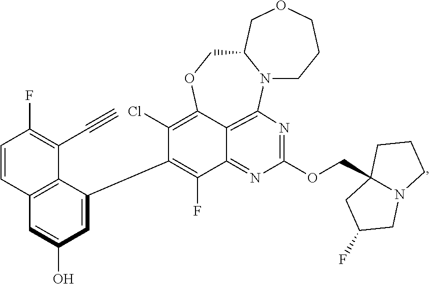

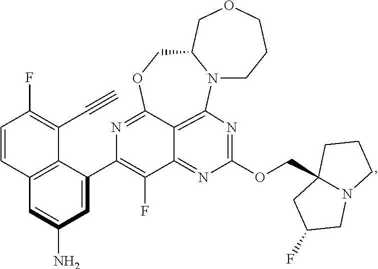

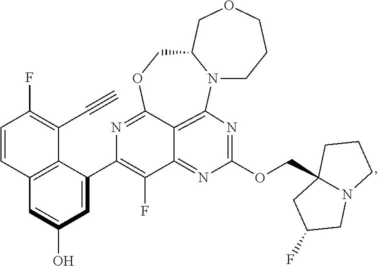

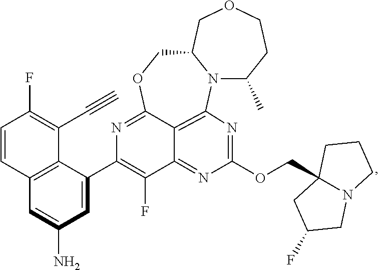

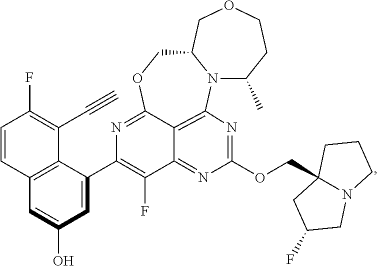

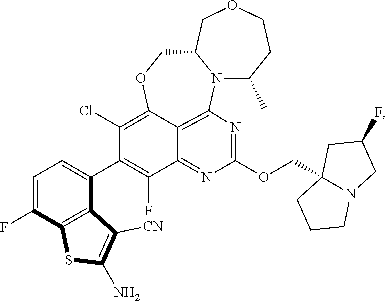

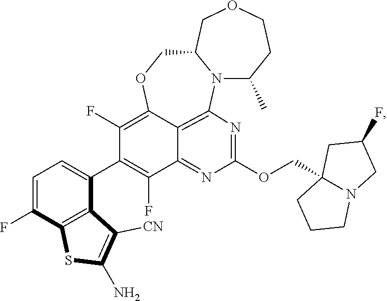

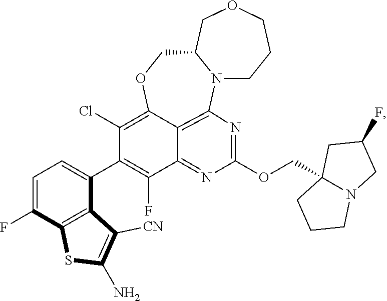

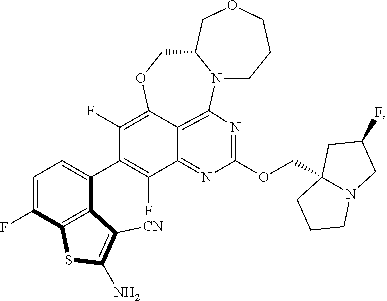

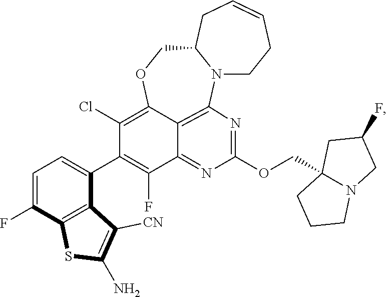

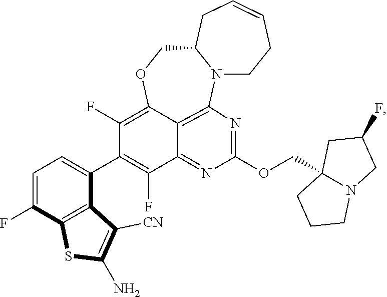

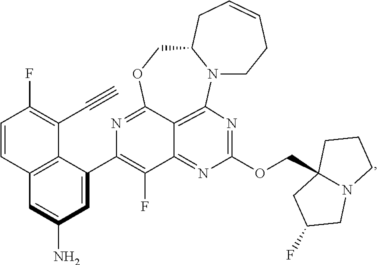

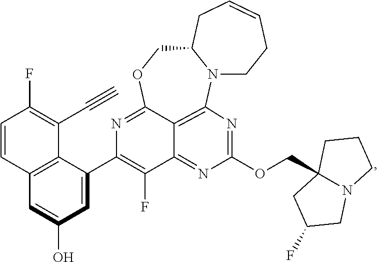

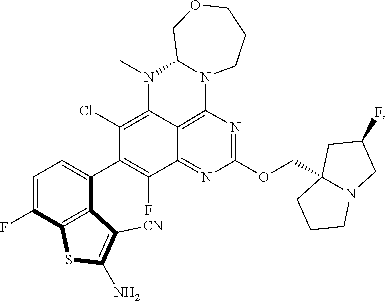

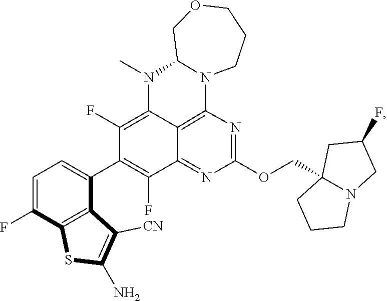

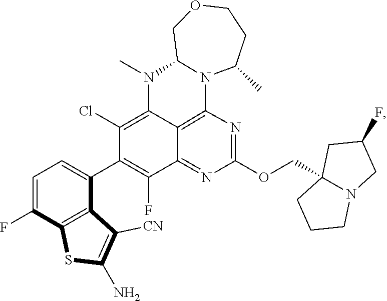

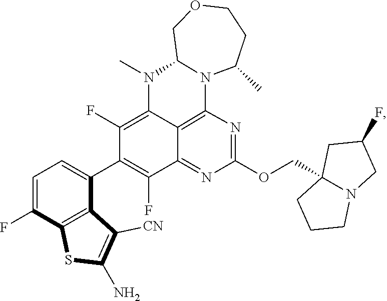

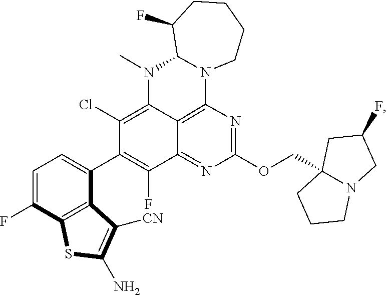

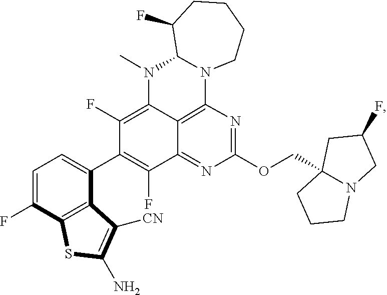

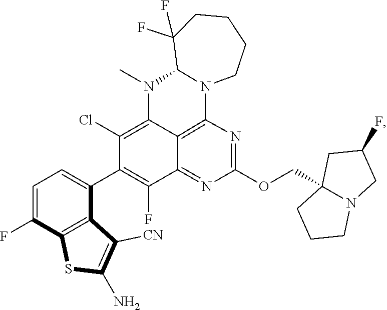

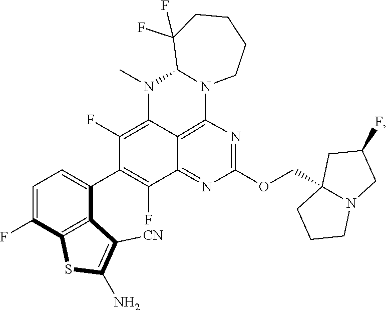

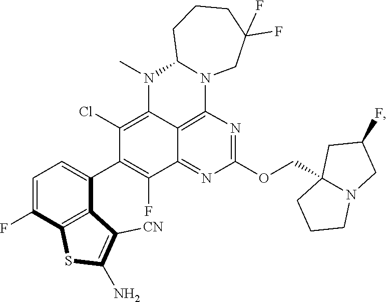

or

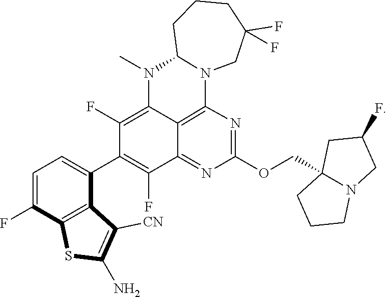

[67]. A pharmaceutical composition, comprising a therapeutically effective amount of the compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [66], and a pharmaceutically acceptable excipient.

[68]. A method for treating cancer in a subject comprising administering a therapeutically effective amount of the compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [66], or the pharmaceutical composition of to a subject in need thereof.

[69]. A method for treating cancer in a subject in need thereof, the method comprising:
(a) determining whether the cancer is associated with K-Ras G12C, K-Ras G12D, K-Ras G12V, K-Ras G13D, K-Ras G12R, K-Ras G12S, K-Ras G12A, K-Ras Q61H mutation and/or K-Ras wild type amplification; and
(b) if so, administering a therapeutically effective amount of the compound of Formula (I), a stereoisomer thereof, a pharmaceutically acceptable salt thereof, a pharmaceutically acceptable salt of the stereoisomer thereof, a prodrug thereof, a deuterated molecule thereof or a PROTAC molecule thereof of any one of [1] to [66], or the pharmaceutical composition of [67] to the subject in need thereof.

[70]. The compound of formula (I), a stereoisomer thereof, a pharmaceutically acceptable salt thereof, a pharmaceutically acceptable salt of the stereoisomer, a prodrug thereof, a deuterated molecule thereof or the PROTAC molecule thereof of any one of [1] to [66], or the pharmaceutical composition of [67] for use in therapy.

[71]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [66], or the pharmaceutical composition of [67] for use as a medicament.

[72]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [66], or the pharmaceutical composition of [67] for use in a method for the treatment of cancer.

[73]. A use of the compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [66], or the pharmaceutical composition of [67] for the treatment of cancer.

[74]. A use of the compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [66], or the pharmaceutical composition of [67] for the manufacture of a medicament for the treatment of cancer.

[75]. The method for treating cancer of [68], the use in a method for the treatment of cancer of [72], the use for the treatment of cancer of [73], or the use for the manufacture of a medicament for the treatment of cancer of [74], wherein, said cancer is pancreatic carcinoma, colorectal carcinoma, lung carcinoma (such as non-small cell lung cancer), breast carcinoma, large intestine carcinoma, stomach carcinoma, endometrial carcinoma, esophageal carcinoma or gastroesophageal junction carcinoma.

[76]. The method for treating cancer of [68] or [75], the use in a method for the treatment of cancer of [72] or [75], the use for the treatment of cancer of [73] or [75], or the use for the manufacture of a medicament for the treatment of cancer of [74] or [75], wherein, the cancer is associated with at least one of K-Ras G12C, K-Ras G12D, K-Ras G12V. K-Ras G13D, K-Ras G12R, K-Ras G12S, K-Ras G12A, K-Ras Q61H mutation and/or K-Ras wild type amplification.

Provided herein is the following:

[B-1]. A compound of formula (I):

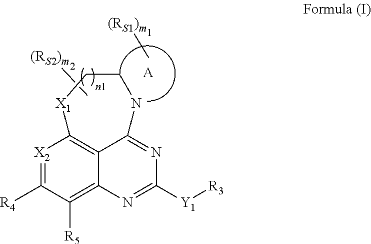

a stereoisomer thereof, a pharmaceutically acceptable salt thereof, a pharmaceutically acceptable salt of the stereoisomer, a prodrug thereof, a deuterated molecule thereof or a PROTAC molecule thereof;
Wherein,
R₃ is —C₁₋₆alkylene, which is optionally substituted with one or more deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}—C(═O)R_{D}, —C(═O)OR_{c}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{c}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Ring A, R_{S1}, m₁, R_{S2}, m₂, n₁, X₁, X₂, R₄, R₅, Y₁, R_{A}, R_{B}, R_{C}, R_{D} has the identical definition of any one of [1] to [66].

[B-2]. The compound of [B-1], wherein, R₃ is —C₁₋₆alkylene substituted with —N(R_{A})₂; R_{A} is hydrogen or —C₁₋₃alkyl.

[B-3]. The compound of [B-2], wherein, R₃ is —C₁₋₆alkylene substituted with —N(CH₃)₂.

Provided herein is the following:

[C-1]. An intermediate having the following structure:

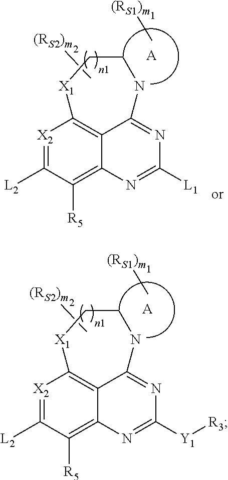

L₁ is a leaving group;
L₂ is a leaving group;
Ring A, R_{S1}, m₁, R_{S2}, m₂, n₁, X₁, X₂, R₅, Y₁, R₃ has the identical definition of any one of [1] to [66] and [B-1] to [B-3].

[C-2]. The intermediate of [C-1], wherein, L₁ is —Cl, —Br, —S(═O)CH₃, or —S(═O)₂CH₃.

[C-3]. The intermediate of [C-1] or [C-2], wherein, L₂ is —Cl or —Br.

[C-4]. The intermediate of any one of [C-1] to [C-3], wherein, —Y₁—R₃ is

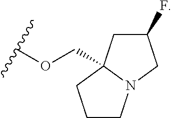 [SMILES: *OC[C@@]12CCCN1C[C@H](F)C2]

[C-5]. The intermediate of any one of [C-1] to [C-4], wherein, the intermediate is any one of intermediates in the Table D:

TABLE D

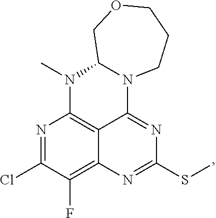

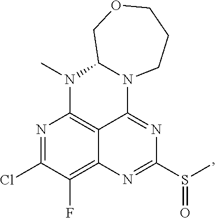

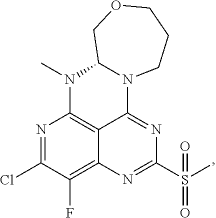

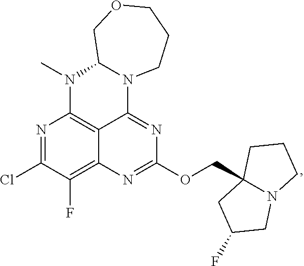 [SMILES: CN1c2nc(Cl)c(F)c3nc(OC[C@@]45CCCN4C[C@H](F)C5)nc(c23)N2CCCOC[C@@H]12 ; ","]

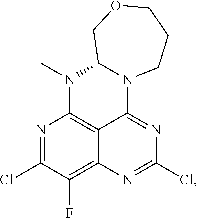 [SMILES: CN1c2nc(Cl)c(F)c3nc(Cl)nc(c23)N2CCCOC[C@@H]12]

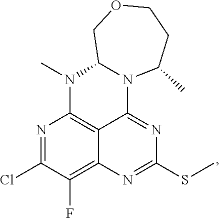 [SMILES: CSc1nc2c3c(nc(Cl)c(F)c3n1)N(C)[C@@H]1COCC[C@H](C)N21 ; ","]

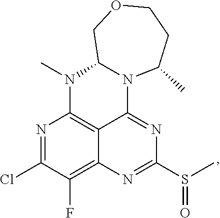 [SMILES: C[C@H]1CCOC[C@H]2N(C)c3nc(Cl)c(F)c4nc(S(C)=O)nc(c34)N12 ; ","]

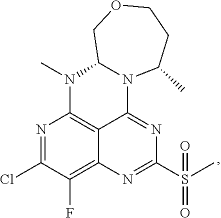 [SMILES: C[C@H]1CCOC[C@H]2N(C)c3nc(Cl)c(F)c4nc(S(C)(=O)=O)nc(c34)N12 ; ","]

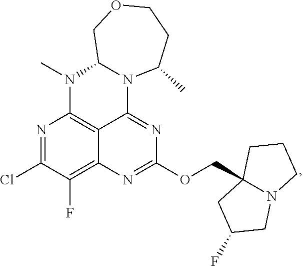 [SMILES: C[C@H]1CCOC[C@H]2N(C)c3nc(Cl)c(F)c4nc(OC[C@@]56CCCN5C[C@H](F)C6)nc(c34)N12 ; ","]

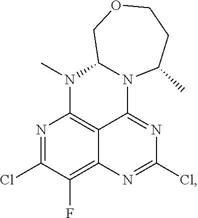 [SMILES: C[C@H]1CCOC[C@H]2N(C)c3nc(Cl)c(F)c4nc(Cl)nc(c34)N12]

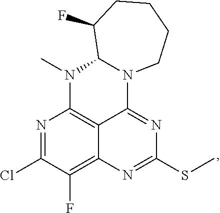 [SMILES: CSc1nc2c3c(nc(Cl)c(F)c3n1)N(C)[C@@H]1[C@@H](F)CCCCN21 ; ","]

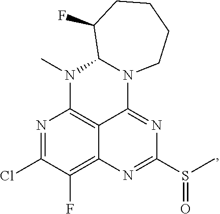 [SMILES: CN1c2nc(Cl)c(F)c3nc(S(C)=O)nc(c23)N2CCCC[C@H](F)[C@@H]12 ; ","]

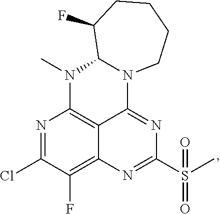 [SMILES: CN1c2nc(Cl)c(F)c3nc(S(C)(=O)=O)nc(c23)N2CCCC[C@H](F)[C@@H]12 ; ","]

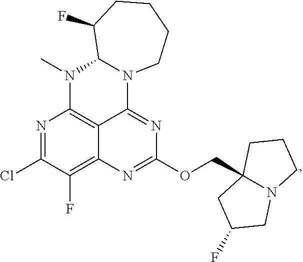 [SMILES: CN1c2nc(Cl)c(F)c3nc(OC[C@@]45CCCN4C[C@H](F)C5)nc(c23)N2CCCC[C@H](F)[C@@H]12 ; ","]

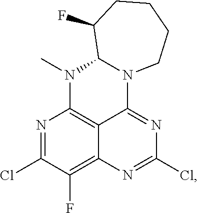 [SMILES: CN1c2nc(Cl)c(F)c3nc(Cl)nc(c23)N2CCCC[C@H](F)[C@@H]12]

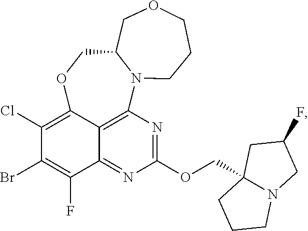 [SMILES: Fc1c(Br)c(Cl)c2c3c(nc(OC[C@@]45CCCN4C[C@H](F)C5)nc13)N1CCCOC[C@H]1CO2]

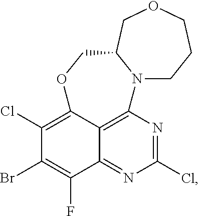 [SMILES: Fc1c(Br)c(Cl)c2c3c(nc(Cl)nc13)N1CCCOC[C@H]1CO2]

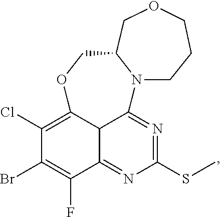

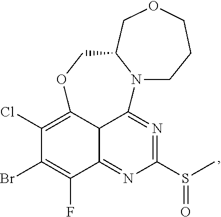

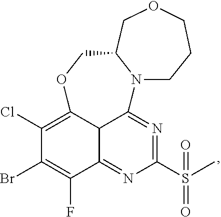

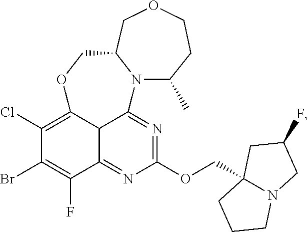

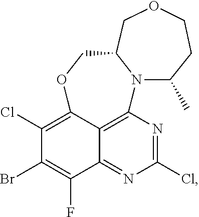 [SMILES: C[C@H]1CCOC[C@H]2COc3c(Cl)c(Br)c(F)c4nc(Cl)nc(c34)N21]

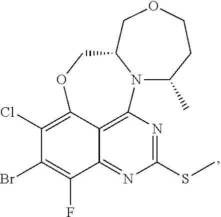 [SMILES: CSc1nc2c3c(c(Cl)c(Br)c(F)c3n1)OC[C@@H]1COCC[C@H](C)N21 ; ","]

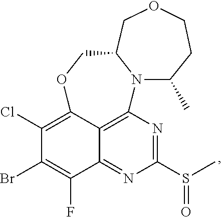 [SMILES: C[C@H]1CCOC[C@H]2COc3c(Cl)c(Br)c(F)c4nc(S(C)=O)nc(c34)N21 ; ","]

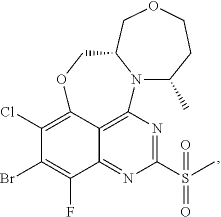 [SMILES: C[C@H]1CCOC[C@H]2COc3c(Cl)c(Br)c(F)c4nc(S(C)(=O)=O)nc(c34)N21 ; ","]

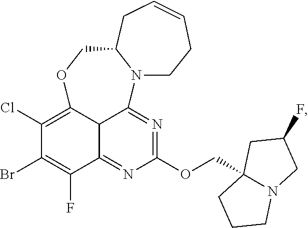

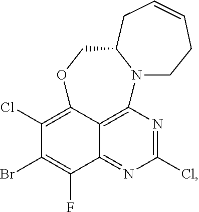 [SMILES: Fc1c(Br)c(Cl)c2c3c(nc(Cl)nc13)N1CCC=CC[C@H]1CO2]

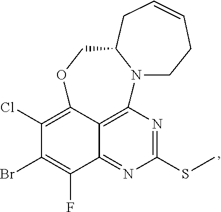 [SMILES: CSc1nc2c3c(c(Cl)c(Br)c(F)c3n1)OC[C@@H]1CC=CCCN21 ; ","]

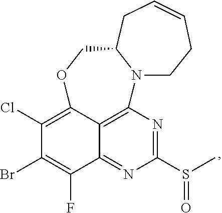 [SMILES: CS(=O)c1nc2c3c(c(Cl)c(Br)c(F)c3n1)OC[C@@H]1CC=CCCN21 ; ","]

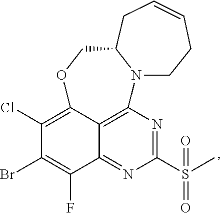 [SMILES: CS(=O)(=O)c1nc2c3c(c(Cl)c(Br)c(F)c3n1)OC[C@@H]1CC=CCCN21 ; ","]

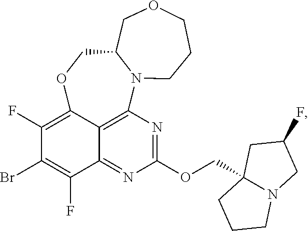 [SMILES: Fc1c(Br)c(F)c2nc(OC[C@@]34CCCN3C[C@H](F)C4)nc3c2c1OC[C@@H]1COCCCN31]

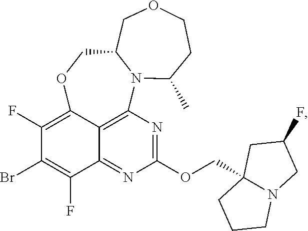 [SMILES: C[C@H]1CCOC[C@H]2COc3c(F)c(Br)c(F)c4nc(OC[C@@]56CCCN5C[C@H](F)C6)nc(c34)N21]

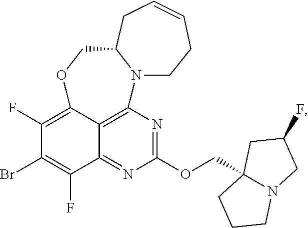 [SMILES: Fc1c(Br)c(F)c2nc(OC[C@@]34CCCN3C[C@H](F)C4)nc3c2c1OC[C@@H]1CC=CCCN31]

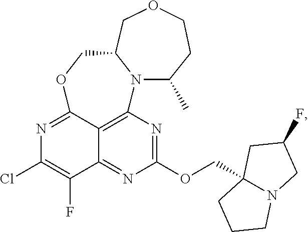 [SMILES: C[C@H]1CCOC[C@H]2COc3nc(Cl)c(F)c4nc(OC[C@@]56CCCN5C[C@H](F)C6)nc(c34)N21]

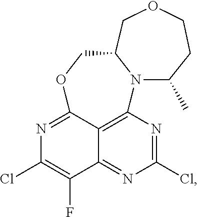 [SMILES: C[C@H]1CCOC[C@H]2COc3nc(Cl)c(F)c4nc(Cl)nc(c34)N21]

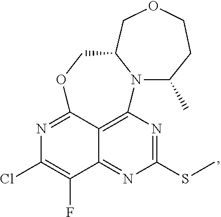 [SMILES: CSc1nc2c3c(nc(Cl)c(F)c3n1)OC[C@@H]1COCC[C@H](C)N21 ; ","]

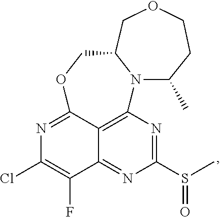 [SMILES: C[C@H]1CCOC[C@H]2COc3nc(Cl)c(F)c4nc(S(C)=O)nc(c34)N21 ; ","]

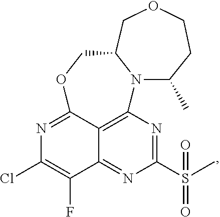 [SMILES: C[C@H]1CCOC[C@H]2COc3nc(Cl)c(F)c4nc(S(C)(=O)=O)nc(c34)N21 ; ","]

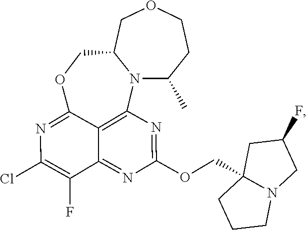 [SMILES: C[C@H]1CCOC[C@H]2COc3nc(Cl)c(F)c4nc(OC[C@@]56CCCN5C[C@H](F)C6)nc(c34)N21]

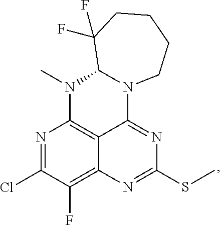 [SMILES: CSc1nc2c3c(nc(Cl)c(F)c3n1)N(C)[C@H]1N2CCCCC1(F)F ; ","]

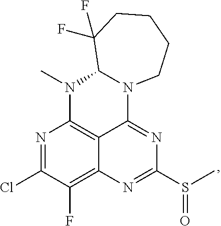 [SMILES: CN1c2nc(Cl)c(F)c3nc(S(C)=O)nc(c23)N2CCCCC(F)(F)[C@@H]12 ; ","]

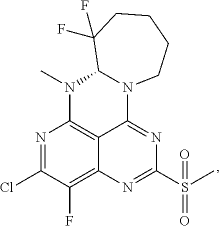 [SMILES: CN1c2nc(Cl)c(F)c3nc(S(C)(=O)=O)nc(c23)N2CCCCC(F)(F)[C@@H]12 ; ","]

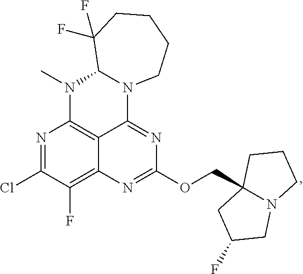 [SMILES: CN1c2nc(Cl)c(F)c3nc(OC[C@@]45CCCN4C[C@H](F)C5)nc(c23)N2CCCCC(F)(F)[C@@H]12 ; ","]

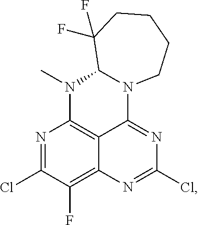 [SMILES: CN1c2nc(Cl)c(F)c3nc(Cl)nc(c23)N2CCCCC(F)(F)[C@@H]12]

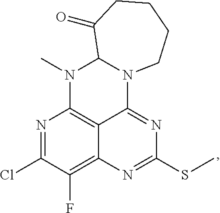 [SMILES: CSc1nc2c3c(nc(Cl)c(F)c3n1)N(C)C1C(=O)CCCCN21 ; ","]

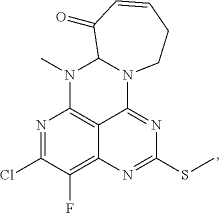 [SMILES: CSc1nc2c3c(nc(Cl)c(F)c3n1)N(C)C1C(=O)C=CCCN21 ; ","]

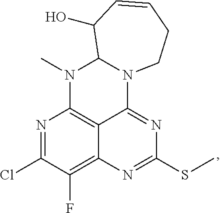 [SMILES: CSc1nc2c3c(nc(Cl)c(F)c3n1)N(C)C1C(O)C=CCCN21 ; ","]

or

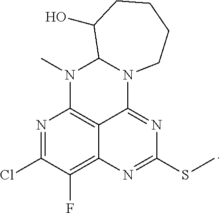 [SMILES: CSc1nc2c3c(nc(Cl)c(F)c3n1)N(C)C1C(O)CCCCN21 ; "."]

### Definition

Unless defined otherwise, all technical and scientific terms used herein have the same meaning as is commonly understood by one of skill in the art to which this invention belongs. All patents, patent applications, and publications referred to herein are incorporated by reference.

The term “halogen” or “halo”, as used herein, unless otherwise indicated, means fluoro, chloro, bromo or iodo. The preferred halogen groups include —F, —Cl and —Br.

The term “alkyl”, as used herein, unless otherwise indicated, includes saturated monovalent hydrocarbon radicals having straight or branched. For example, —C₁₋₆alkyl radicals include methyl, ethyl, propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl. Similarly, C₁₋₃, as in —C₁₋₃alkyl is defined to identify the group as having 1, 2, or 3 carbon atoms in a linear or branched arrangement.

The term “haloalkyl” (such as —C₁₋₆haloalkyl, —C₁₋₄haloalkyl, —C₁₋₃haloalkyl, or haloC₁₋₆alkyl) as used herein, unless otherwise indicated, the alkyl (such as —C₁₋₆alkyl, —C₁₋₄alkyl or —C₁₋₃alkyl) as defined herein in which one or more (such as one, two or three) hydrogen has been replaced by a halogen. Examples include trifluoromethyl, difluoromethyl and fluoromethyl.

The term “alkylene” means a difunctional group obtained by removal of a hydrogen atom from an alkyl group defined above. For example, methylene (i.e., —CH₂—), ethylene (i.e., —CH₂—CH₂— or —CH(CH₃)—) and propylene (i.e., —CH₂—CH₂—CH₂—, —CH(—CH₂—CH₃)— or —CH₂—CH(CH₃)—).

The term “alkenyl” means a straight or branch-chained hydrocarbon radical containing one or more double bonds and typically from 2 to 20 carbon atoms in length. For example, “—C₂₋₆alkenyl” is the Alkenyl containing 2 to 6 of carbon atoms. Alkenyl group include, but are not limited to, for example, ethenyl, propenyl, butenyl, 2-methyl-2-buten-1-yl, heptenyl, octenyl and the like.

The term “alkynyl” contains a straight or branch-chained hydrocarbon radical containing one or more triple bonds and typically from 2 to 20 carbon atoms in length. For example, “—C₂₋₆alkynyl” is the alkynyl containing 2 to 6 of carbon atoms. Representative alkynyl groups include, but are not limited to, for example, ethynyl, 1-propynyl, 1-butynyl, heptynyl, octynyl and the like.

The term “alkoxy” radicals are oxygen ethers formed from the previously described alkyl groups.

The term “aryl”, as used herein, unless otherwise indicated, refers to an unsubstituted or substituted mono or polycyclic aromatic ring system containing carbon ring atoms. The preferred aryls are mono cyclic or bicyclic 6-10 membered aromatic ring systems. Phenyl and naphthyl are preferred aryls.

The term “heterocyclic” or “heterocyclyl”, as used herein, unless otherwise indicated, refers to unsubstituted and substituted mono or polycyclic non-aromatic ring system containing one or more heteroatoms, which comprising monocyclic heterocyclic ring, bicyclic heterocyclic ring, bridged heterocyclic ring, fused heterocyclic ring or spiro heterocyclic ring. Preferred heteroatoms include N, O, and S, including N-oxides, sulfur oxides, and dioxides. Preferably, the ring is three to ten membered and is either fully saturated or has one or more degrees of unsaturation. Multiple degrees of substitution, preferably one, two or three, are included within the present definition. Examples of such heterocyclic groups include, but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinylsulfoxide, thiamorpholinylsulfone and oxadiazolyl.

The term “heteroaryl”, as used herein, unless otherwise indicated, represents an aromatic ring system containing carbon(s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 10 hetero atoms. A polycyclic heteroaryl ring may contain fused, spiro or bridged ring junction, for example, bycyclicheteroaryl is a polycyclic heteroaryl. Bicyclic heteroaryl rings may contain from 8 to 12 member atoms. Monocyclic heteroaryl rings may contain from 5 to 8 member atoms (carbons and heteroatoms). Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyladeninyl, quinolinyl or isoquinolinyl.

The term “carbocyclic” refers to a substituted or unsubstituted monocyclic ring, bicyclic ring, bridged ring, fused ring, spiro ring non-aromatic ring system only containing carbon atoms. Exemplary “cycloalkyl” groups include but not limited to, cyclopropyl, cyclobutyl, cyclopentyl, cyclohexyl and so on.

The term “one or more”, as used herein, unless otherwise indicated, refers to one or more than one. In some embodiments, “one or more” refers to 1, 2, 3, 4, 5 or 6. In some embodiments, “one or more” refers to 1, 2, 3 or 4. In some embodiments. “one or more” refers to 1, 2, or 3. In some embodiments, “one or more” refers to 1 or 2. In some embodiments, “one or more” refers to 1. In some embodiments, “one or more” refers to 2. In some embodiments, “one or more” refers to 3. In some embodiments, “one or more” refers to 4. In some embodiments, “one or more” refers to 5. In some embodiments, “one or more” refers to 6.

The term “substituted”, as used herein, unless otherwise indicated, refers to a hydrogen atom on the carbon atom or a hydrogen atom on the nitrogen atom is replaced by a substituent. When one or more substituents are substituted on a ring in the present invention, it means that each of substituents may be respectively independently substituted on every ring atom of the ring including but not limited to a ring carbon atom or a ring nitrogen atom. In addition, when the ring is a polycyclic ring, such as a fused ring, a bridged ring or a spiro ring, each of substituents may be respectively independently substituted on every ring atom of the polycyclic ring.

The term “oxo” refers to oxygen atom together with the attached carbon atom form the group

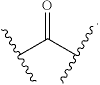 [SMILES: *C(*)=O]

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The term “composition”, as used herein, is intended to encompass a product comprising the specified ingredients in the specified amounts, as well as any product which resulted, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

The term “pharmaceutically acceptable salt” refers to a salt prepared from pharmaceutically acceptable non-toxic bases or acids. When the compound of the present invention is acidic, its corresponding salt can be conveniently prepared from pharmaceutically acceptable non-toxic bases, including inorganic bases and organic bases. When the compound of the present invention is basic, its corresponding salt can be conveniently prepared from pharmaceutically acceptable non-toxic acids, including inorganic and organic acids. Since the compounds in the present invention are intended for pharmaceutical use they are preferably provided in substantially pure form, for example at least 60% pure, more suitably at least 75% pure, especially at least 98% pure (% are on a weight for weight basis).

The present invention includes within its scope the prodrug of the compounds of this invention. In general, such prodrug will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes all stereoisomers of the compound and pharmaceutically acceptable salts thereof. Further, mixtures of stereoisomers as well as isolated specific stereoisomers are also included. During the course of the synthetic procedures used to prepare such compounds or in using racemization or epimerization procedures known to those skilled in the art, the products of such procedures can be a mixture of stereoisomers. The term “stereoisomer” as used in the present invention refers to an isomer in which atoms or groups of atoms in the molecule are connected to each other in the same order but differ in spatial arrangement, including conformational isomers and configuration isomers. The configuration isomers include geometric isomers and optical isomers, and optical isomers mainly include enantiomers and diastereomers. The invention includes all possible stereoisomers of the compound (such as atropisomer). Absolute configuration of the compound of the present invention can be confirmed through general technical methods such as X-ray single crystal diffraction or co-crystal with a KRAS mutant protein, or through the comparison of pharmacological activities of two isomers to the pharmacological activities of a pair of isomers with confirmed absolute configuration.

The present invention is intended to include all isotopes of atoms occurring in the present compounds. Isotopes include those atoms having the same atomic number but different mass numbers. By way of general example and without limitation, isotopes of hydrogen include deuterium and tritium. The isotopes of hydrogen can be denoted as ¹H (hydrogen), ²H (deuterium) and ³H (tritium). They are also commonly denoted as D for deuterium and T for tritium. In the application, CD₃ denotes a methyl group wherein all of the hydrogen atoms are deuterium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent.

The term “deuterated derivative”, used herein, unless otherwise indicated, refers to a compound having the same chemical structure as a reference compound, but with one or more hydrogen atoms replaced by a deuterium atom (“D”). It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivative described herein. Thus, unless otherwise stated, when a reference is made to a “deuterated derivative” of a compound of the disclosure, at least one hydrogen is replaced with deuterium at well above its natural isotopic abundance (which is typically about 0.015%)

When a tautomer of the compound in the present invention exists, the present invention includes any possible tautomer and pharmaceutically acceptable salts thereof, and mixtures thereof, except where specifically stated otherwise.

The “PROTAC molecule” refers to herein that the compound described herein is conjugated to another agent through a linker or not through a linker, wherein, the compound functions as a binder or an inhibitor of K-Ras protein (including K-Ras G12C, K-Ras G12D, K-Ras G12V, K-Ras G13D, K-Ras G12R, K-Ras G12S, K-Ras G12A, K-Ras Q61H mutant protein and K-Ras wild type protein), e.g. the compound is incorporated into proteolysis targeting chimeras (PROTACs).

Unless otherwise apparent from the context, when a value is expressed as “about” X or “approximately” X, the stated value of X will be understood to be accurate to ±10%, preferably, ±5%, ±2%.

The term “subject” refers to an animal. In some embodiments, the animal is a mammal. A subject also refers to for example, primates (e.g., humans), cows, sheep, goats, horses, dogs, cats, rabbits, rats, mice, fish, birds and the like. In certain embodiments, the subject is a human. A “patient” as used herein refers to a human subject. As used herein, a subject is “in need of” a treatment if such subject would benefit biologically, medically or in quality of life from such treatment. In some embodiments, the subject has experienced and/or exhibited at least one symptom of cancer to be treated and/or prevented. In some embodiments, the subject has been identified or diagnosed as having a cancer having wild type K-Ras or a K-Ras G12A, K-Ras G12C, K-Ras G12D, K-Ras G12R, K-Ras G12S, K-Ras G12V, K-Ras G13D and/or K-Ras Q61H mutation

The term “inhibition”. “inhibiting” or “inhibit” refers to the reduction or suppression of a given condition, symptom, or disorder, or disease, or a significant decrease in the baseline activity of a biological activity or process.

The term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment, “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment. “treat”, “treating” or “treatment” refers to preventing or delaying the onset or development or progression of the disease or disorder.

### EXAMPLES

The following examples are provided to better illustrate the present invention. All parts and percentages are by weight and all temperatures are degrees Celsius, unless explicitly stated otherwise. The following abbreviations have been used in the examples:

| | |
|---|---|
| DMF | N,N-dimethylformamide |
| EA/EtOAc | Ethyl acetate |
| Hex | Hexane |
| MeOH | Methanol |
| DCM | Dichloromethane |
| DCE | 1,2-dichloroethane |
| EtOH | Ethanol |
| THF | Tetrahydrofuran |
| DIEA/DIPEA | N,N-Diisopropylethylamine |
| Pd(PPh₃)₄ | Tetrakis(triphenylphosphine)palladium |
| Pd(dppf)Cl₂ | [1,1′-Bis(diphenylphosphino)ferrocene]dichloropalladium(II) |
| TFA | 2,2,2-trifluoroacetic acid |
| ACN/MeCN/CH₃CN | Acetonitrile |
| Et₃N/TEA | Triethylamine |
| NIS | N-Iodosuccinimide |
| DMSO | Dimethyl sulfoxide |
| NCS | N-chlorosuccinimide |
| TBSCl | Tert-butyldimethylsilyl chloride |
| TMSCl | Trimethylsilyl chloride |
| MOMCl | Methoxymethyl chloride |
| MsCl | Methanesulfonyl chloride |
| LAH | Lithium aluminum hydride |
| LDA | Lithium diisopropylamide |
| LiHMDS | Lithium hexamethyldisilazide |
| B₂(Pin)₂ | Bis(pinacolato)diboron |
| NFSI | N-Fluorobenzenesulfonimide |
| MTBE | Methyl tert-butyl ether |
| DMAP | N,N-dimethylpyridin-4-amine |
| DABCO | Triethylenediamine |
| TBAF | Tetrabutylammonium fluoride |
| m-CPBA | 3-Chloroperbenzoic acid |
| NMP | N-methylpyrrolidone |
| rt/RT/R.T | Room temperature |
| min(s) | minute(s) |
| h/hr(s) | hour(s) |
| aq | aqueous |
| Sat. | saturated |
| TLC | Thin layer chromatography |
| Prep-TLC | Preparative thin layer chromatography |
| MOMO | Methoxymethoxyl |
| TIPS | Triisopropylsilyl |
| IPA | Isopropyl alcohol |
| cataCXium A Pd G3 | Methanesulfonato(diadamantyl-n-butylphosphino)-2′-amino-1,1′- |
| | biphenyl-2-yl)palladium(II) |
| 4A MS | 4A Molecular sieve |
| CP | Compound |
| RT | Room temperature |
| Confirmed | The absolute configuration of the compound was confirmed. |
| Assumed | The absolute configuration of the compound was assumed. |
| Grubbs Gen 2nd | GRUBBS CATALYST 2ND GENERATION |
| DMAc | N,N-Dimethylacetamide |
| Xantphos | 4,5-Bis(diphenylphosphino)-9,9-dimethylxanthene |
| DEA | diethylamine |

The above intermediate was purchased or prepared by prior art.

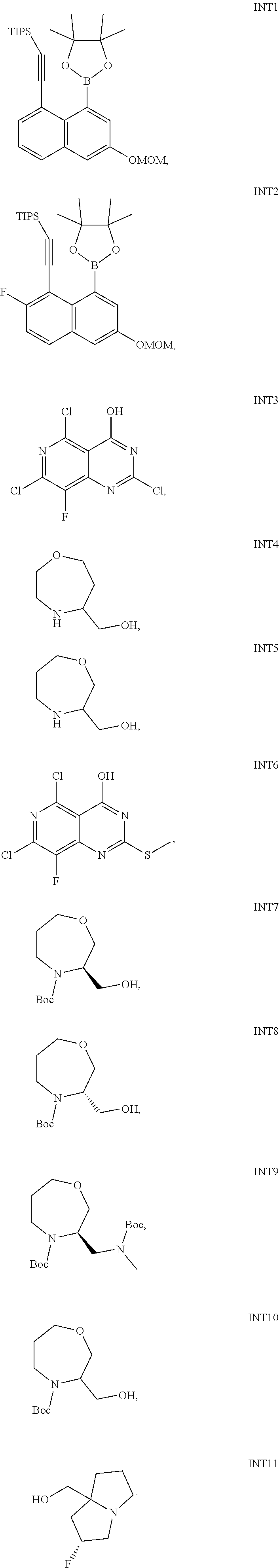

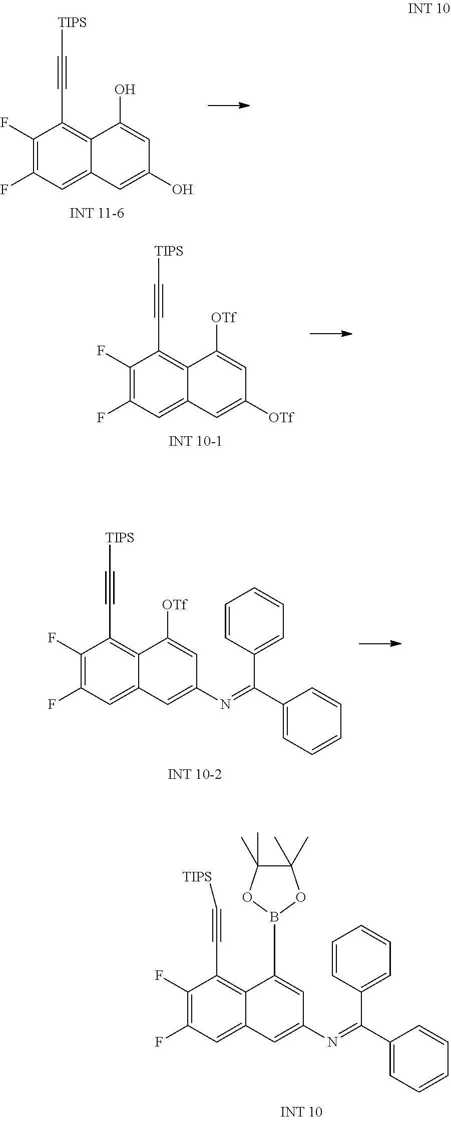

To a solution of INT11-6 (8.2 g) in DCM (100 mL) was added DIEA (11.82 g), trifluoromethanesulfonic anhydride (15.77 g) was dropwised at 0° C. The reaction mixture was stirred at RT overnight under N₂, adjusted to pH 8 with NaHCO₃ solution and extracted with DCM. The organic layer was post processed and purified to give INT10-1 (9.24 g).

A solution of INT10-1 (9.24 g), benzophenone imine (3.34 g), Cs₂CO₃ (14.87 g), Pd₂(dba)₃ (0.90 g) and Xantphos (1.7 g) in 1,4-dioxane (100 mL) was stirred 80° C. overnight under N₂. The solution was extracted with EA. The separated organic layer was post processed and purified to give INT10-2 (5.71 g).

A solution of INT10-2 (5.71 g), KOAc (2.67 g), bis(pinacolato)diboron (4.24 g) and Pd(dppf)Cl₂ (0.66 g) in toluene (60 mL) was stirred 120° C. overnight under N₂. The solution was extracted with EA. The separated organic layer was post processed and purified to give INT10 (3.02 g).

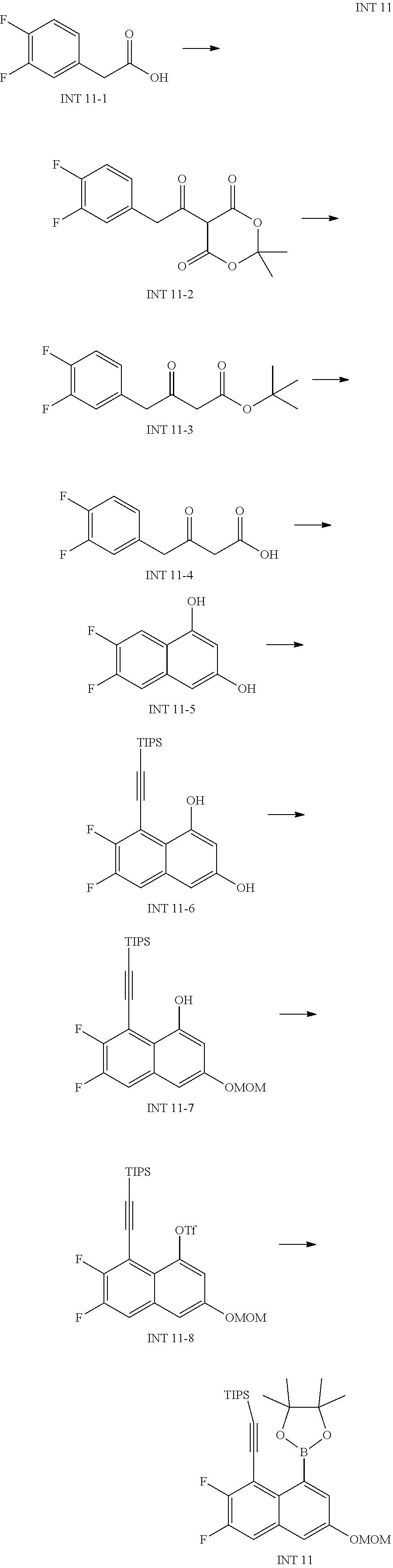

INT 11 was prepared the similar procedure for the synthesis of INT 2 in the WO2021041671.

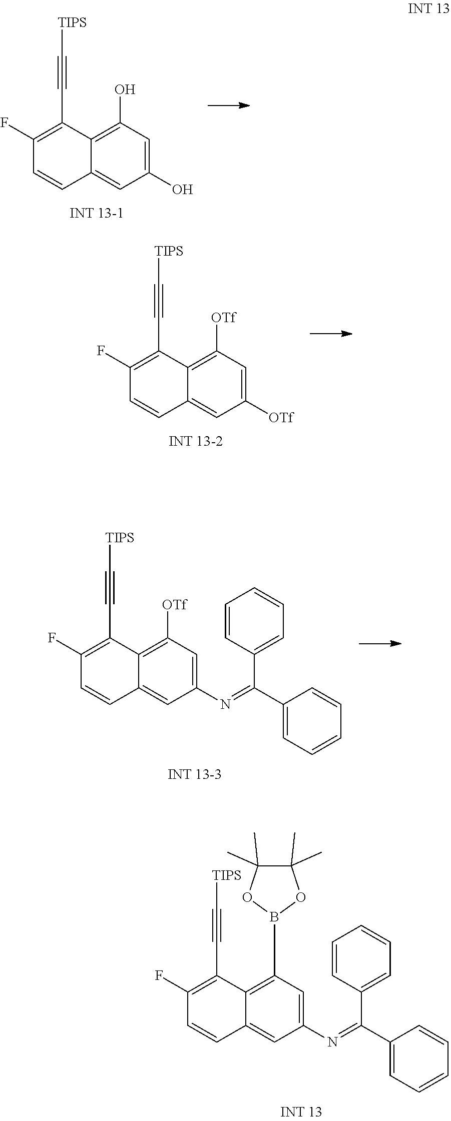

INT 13 was prepared the similar procedure for the synthesis of INT 10.

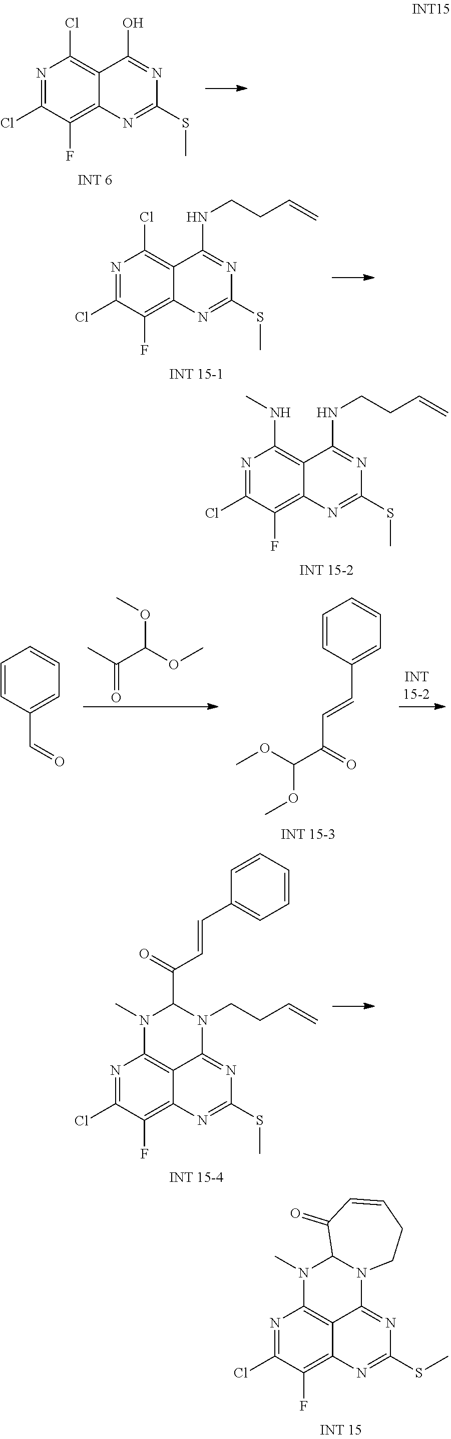

To a solution of INT6 (4.03 g) and DIEA (8 mL) in toluene (25 mL) was added POCl₃ (4 mL) under N₂ and the resulting mixture was stirred at 80° C. for 2 hours and concentrated. The residue was diluted with DCM (40 mL), DIEA (3 mL) and but-3-en-1-amine hydrochloride (1.65 g) was added. The reaction mixture was stirred at RT for 1 hour, diluted with DCM and H₂O. The separated organic layer was concentrated and purified to give INT15-1 (4.61 g). MS: m/z: 333[M+H]⁺.

To a solution of INT15-1 (4.61 g) and methanamine hydrochloride (2.86 g) in DMAc (40 mL) was added DIEA (9.07 g). The mixture was purged with nitrogen followed by stirring at 70° C. for 16 hours, diluted with EA and H₂O. The separated organic layer was concentrated and purified to give INT15-2 (3.60 g). MS: m/z: 328[M+H]⁺.

To a solution of benzaldehyde (1.00 g) in MeOH (20 mL) were added piperidine (2.34 g) and 1,1-dimethoxypropan-2-one (4.36 g). The mixture was purged with nitrogen followed by stirring at rt overnight, diluted with EA and washed with water. The separated organic layer was concentrated and purified to give INT15-3 (1.0 g). MS: m/z: 207[M+H]⁺.

To a solution of INT15-3 (0.31 g) and INT 15-2 (0.38 g) in DMSO (5 mL) was added TsOH·H₂O (0.30 g). The mixture was purged with nitrogen followed by stirring at 80° C. overnight, cooled to RT, diluted with EA and washed with water. The separated organic layer was concentrated and purified to give INT15-4 (0.48 g). MS: m/z: 470[M+H]⁺.

To a solution of INT15-4 (1.12 g) in toluene (20 mL) was added Grubbs Gen 2nd (0.42 g). The mixture was purged with nitrogen followed by stirring at 80° C. for 2 h, cooled to RT and concentrated. The residue was purified to give INT15 (0.28 g). MS: m/z: 366[M+H]⁺.

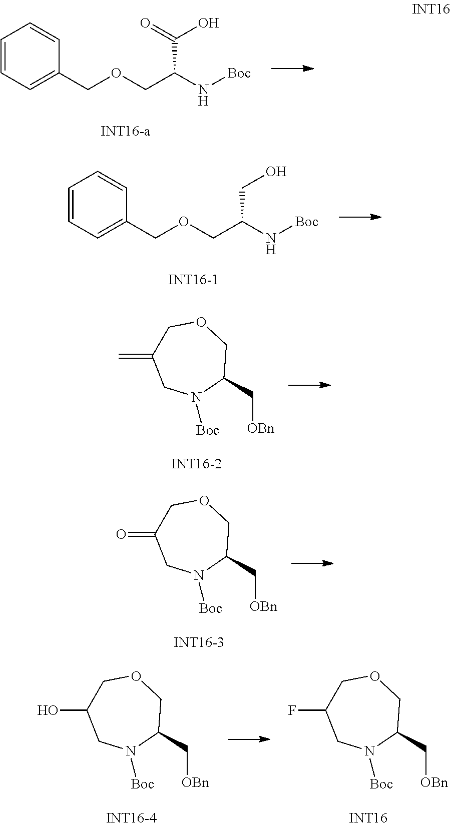

A solution of INT16-a (70.59 g) and TEA (29.70 g) in THF (1000 mL) was cooled to −15° C. and to the resulting mixture was added isobutyl carbonochloridate (36.31 g), stirred for 2 hours at the same temperature and filtered. The filtrate was cooled to 0° C. and added to a solution of NaBH₄ (16.71 g) in water (200 mL) dropwise. The solution was stirred for 1 hour at 0° C., quenched by water and extracted with EA. The organic layer was post processed and purified to give INT 16-1 (74.35 g). MS (ESI, m/z): 282[M+H]⁺.

To a solution of 3-chloro-2-(chloromethyl)prop-1-ene (1.92 g) in DMF (15 mL) was added sodium hydride (60% in oil, 1.77 g) under nitrogen at 0° C. and the resulting mixture was stirred at RT for 30 minutes, a solution of INT16-1 (3.82 g) in dry THF (15 mL) was added to the reaction mixture and stirred at RT for 18 hours, diluted with EA and water. The separated organic layer was post processed and purified to give INT16-2 (2308 mg). MS: m/z: 334[M+H]⁺.

To a solution of INT16-2 (2308 mg) and K₂OsO₄·2H₂O (0.13 g) in THF (25 mL) and water (12 mL) was added NaIO₄ (6.06 g). The mixture was stirred at RT for 18 hours, diluted with EA and water. The separated organic layer was post processed and purified to give INT16-3 (2165 mg). MS: m/z: 336[M+H]⁺.

To a solution of INT16-3 (2165 mg) in THF (15 mL) was added NaBH₄ (495 mg) under nitrogen at −20° C. and the resulting mixture was stirred for 1 hour, diluted with EA and saturated NH₄Cl. The separated organic layer was post processed and purified to give INT16-4 (2.02 g). MS: m/z: 338[M+1]⁺.

To a solution of INT16-4 (2.02 g) in DCM (15 mL) was added BAST (495 mg) under nitrogen at −20° C. and the resulting mixture was stirred at RT for 18 hours, diluted with DCM and saturated NaHCO₃. The separated organic layer was concentrated and purified to give INT16 (0.98 g). MS: m/z: 340[M+1]⁺.

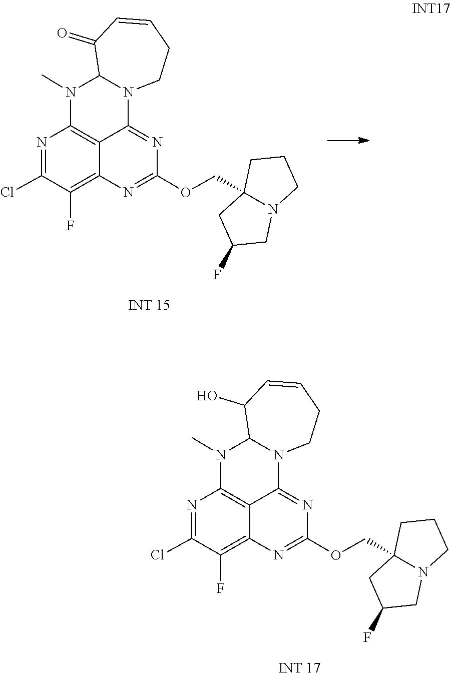 [SMILES: CN1c2nc(Cl)c(F)c3nc(OC[C@]45CCCN4C[C@@H](F)C5)nc(c23)N2CCC=CC(=O)C12 ; CN1c2nc(Cl)c(F)c3nc(OC[C@]45CCCN4C[C@@H](F)C5)nc(c23)N2CCC=CC(O)C12]

To a solution of INT15 (2.02 g) in MeOH (200 mL) and DCM (300 mL) were added NaBH₄ (0.64 g) and CeCl₃·7H₂O (6.12 g) at 0° C. and the resulting mixture was stirred for 4 hour, quenched with H₂O and extracted with EA. The organic layer was post processed to give INT17 (2.02 g, crude). MS: m/z: 479[M+1]⁺.

### Example 1

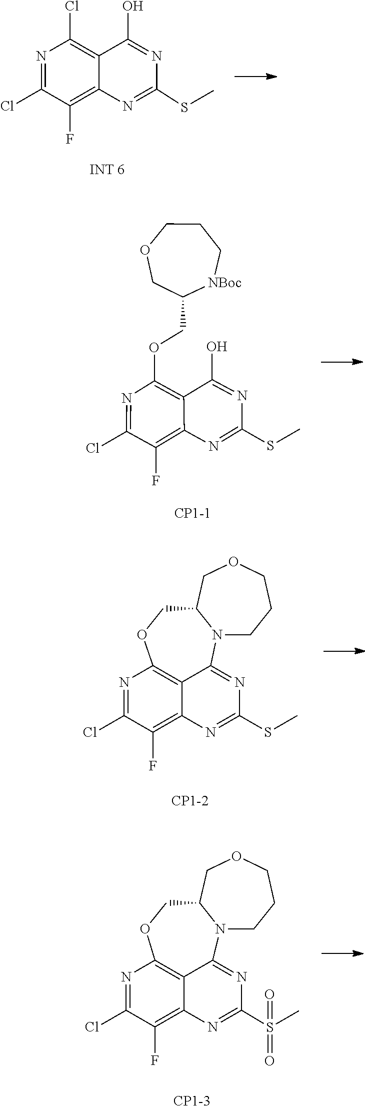 [SMILES: CSc1nc(O)c2c(Cl)nc(Cl)c(F)c2n1 ; CSc1nc(O)c2c(OC[C@@H]3COCCCN3C(=O)OC(C)(C)C)nc(Cl)c(F)c2n1 ; CSc1nc2c3c(nc(Cl)c(F)c3n1)OC[C@@H]1COCCCN21 ; CS(=O)(=O)c1nc2c3c(nc(Cl)c(F)c3n1)OC[C@@H]1COCCCN21]

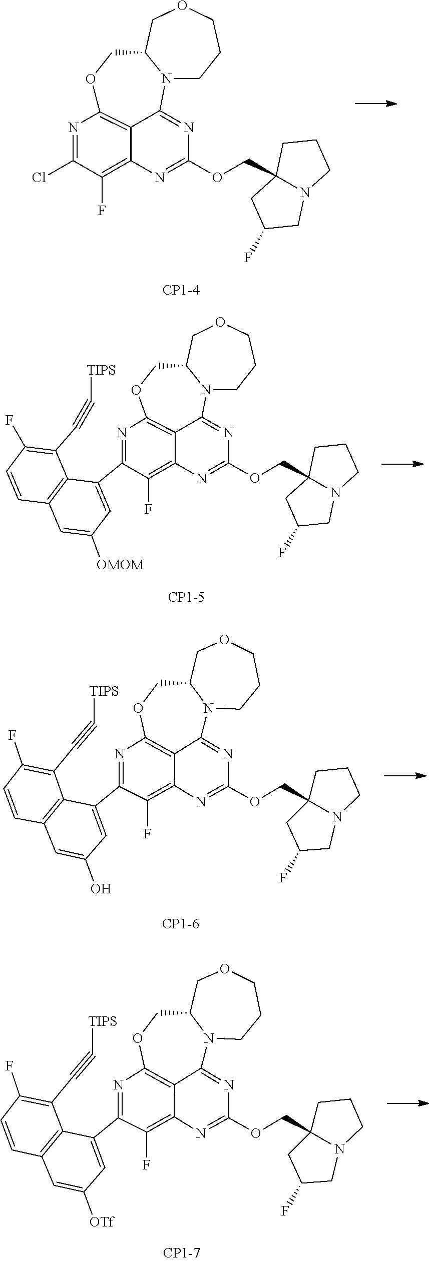

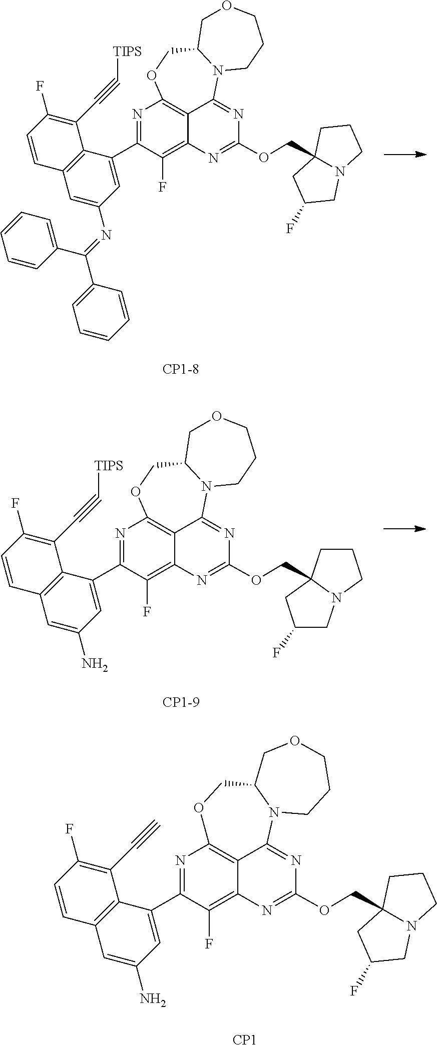

CP1-5 was prepared in a manner similar to the procedure of WO2023046135.

To a solution of CP1-5 (296 mg) in CH₃CN (15 mL) was added HCl (4M in 1,4-dioxane, 5 mL). The reaction mixture was stirred at RT for 1 hour and concentrated. The residue was dissolved in EA and washed with aq. NaHCO₃ (sat.). The organic layer was dried and concentrated to give CP1-6 (316 mg). MS (ESI, m/z): 774[M+H]⁺.

A solution of CP1-6 (292 mg) and pyridine (360 mg) in DCM (6 mL) was cooled to −20° C. T, trifluoromethanesulfonic anhydride (573 mg) was added dropwise and the mixture was warmed to RT. The reaction mixture was stirred at RT for 2 hours and concentrated. The residue was dissolved in EA and washed with water. The organic layer was dried, concentrated and purified by Prep-TLC (DCM:MeOH=30:1 v/v) to afford CP1-7 (251 mg). MS (ESI, m/z): 906[M+H]⁺.

A solution of CP1-7 (51 mg), benzophenone imine (24 mg), Pd₂(dba)₃ (12 mg), Xantphos (13 mg) and Cs₂CO₃ (55 mg) in toluene (3 mL) was stirred at 100° C. for 16 hours under N₂ and diluted with EA and washed with water. The organic layer was dried, concentrated and purified by Prep-TLC (hex:EA=0:1, v/v) to afford CP1-8 (49 mg). MS (ESI, m/z): 937[M+H]⁺.

To a solution of CP1-8 (49 mg) in DCM (3 mL) was added HC (4M in 1,4-dioxane, 0.3 mL). The reaction mixture was stirred at RT for 16 hours and concentrated. The residue was dissolved in EA and washed with sat. aq. NaHCO₃. The organic layer was dried and concentrated to afford CP1-9 (53 mg). MS (ESI, m/z): 773[M+H]⁺.

To a solution of CP1-9 (53 mg) in DMF (3 mL) was added CsF (105 mg). The reaction mixture was stirred at 40° C. for 2 hours, filtered to collect the filtrate. The filtrate was concentrated and purified by Prep-HPLC (Ultimate XB-C18, phase A: 0.05% TFA in water, phase B: CH₃CN, Gradient: 10% B to 28% B in 25 min at a flow rate of 40 mL/min, 228 nm) to afford Compound 1 (CP1, Confirmed). MS (ESI, m/z): 617[M+H]⁺.

### Example 2

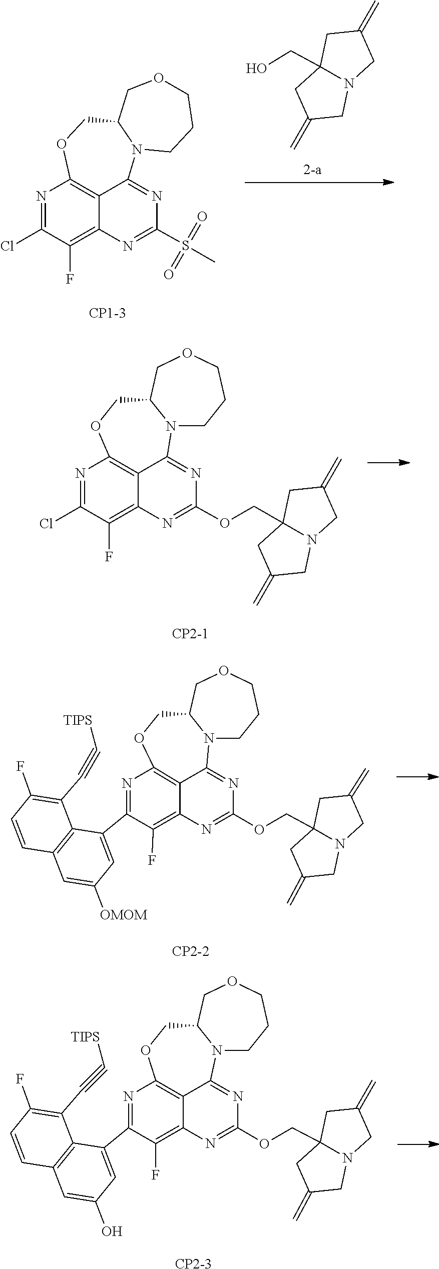

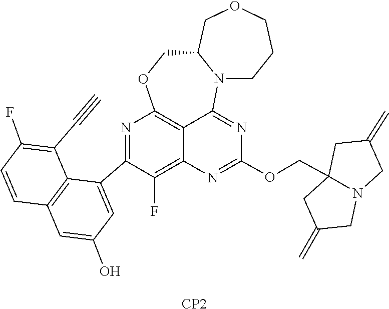 [SMILES: C#Cc1c(F)ccc2cc(O)cc(-c3nc4c5c(nc(OCC67CC(=C)CN6CC(=C)C7)nc5c3F)N3CCCOC[C@H]3CO4)c12 ; "CP2"]

Compound 2 (CP2, 38.5 mg, TFA salt, Confirmed) was synthesized in a manner similar to CP1 with 2-a. MS (ESI, m/z): 624[M+H]⁺.

### Example 3

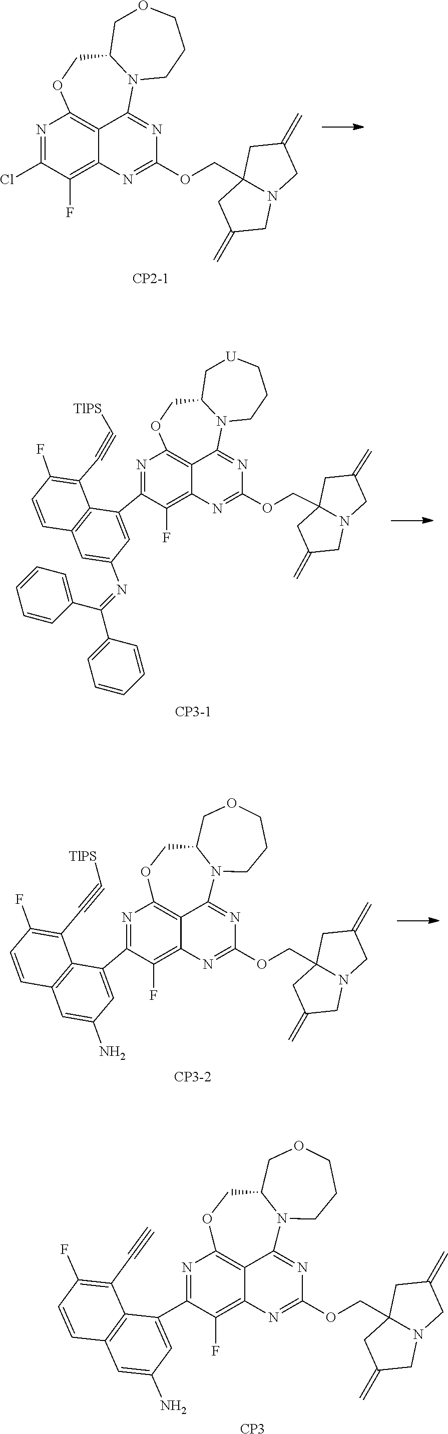

To a solution of CP2-1 (103 mg) and INT13 (337 mg) in toluene (7.5 mL) and water (1.5 mL) were added Cs₂CO₃ (147 mg) and cataCXium A Pd G3 (18 mg). The reaction mixture was stirred at 100° C. overnight under N₂, post processed and purified by Prep-TLC to give CP3-1 (65 mg). MS: m/z: 943[M+H]⁺.

Compound 3 (CP3, 19.5 mg. TEA salt, Confirmed) was obtained by similar procedure of preparing CP1 with CP3-1. MS (ESI, m/z): 623[M+H]⁺.

### Example 4

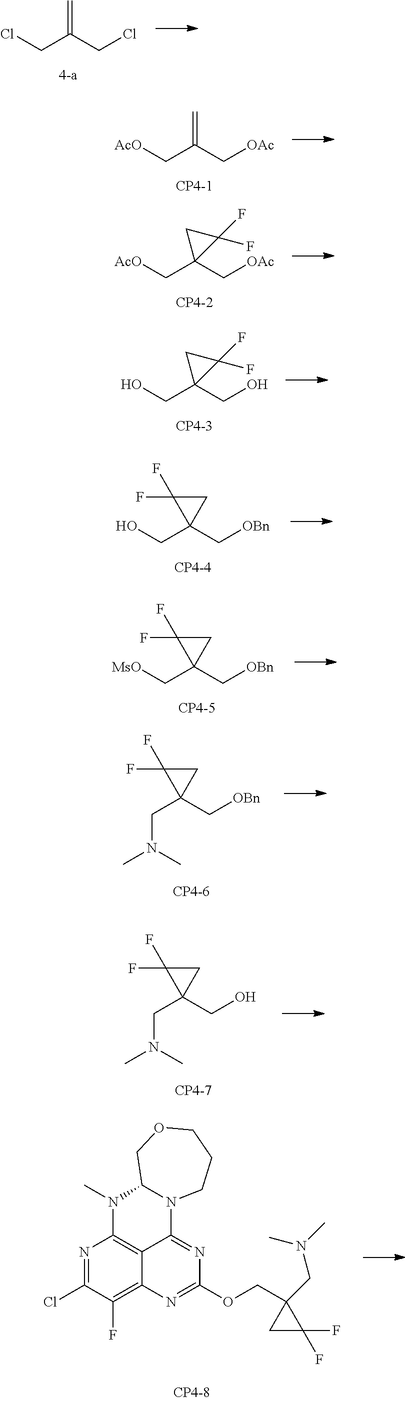

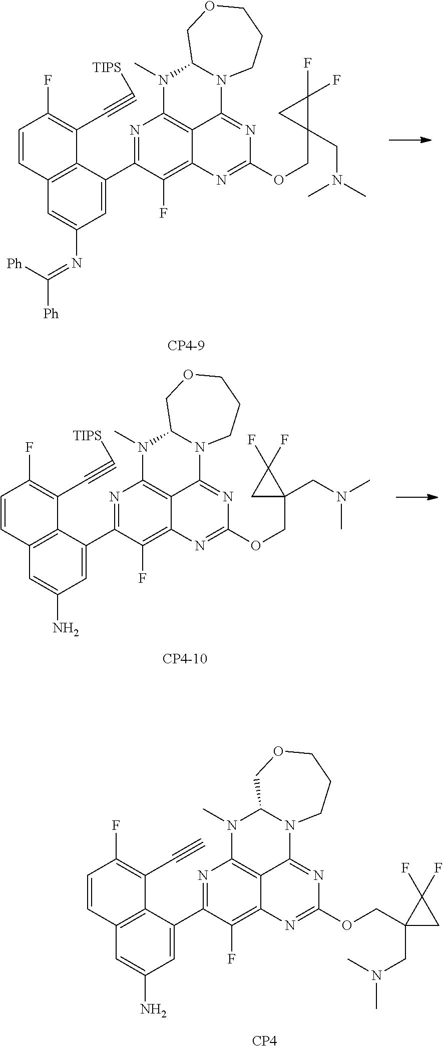

Compound 4 (CP4, confirmed) was synthesized in a manner similar to CP9.

### Example 5

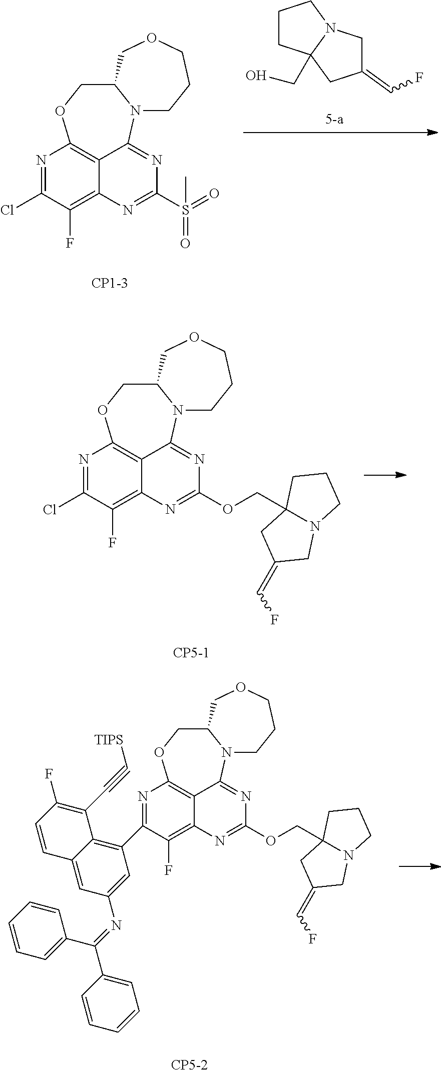

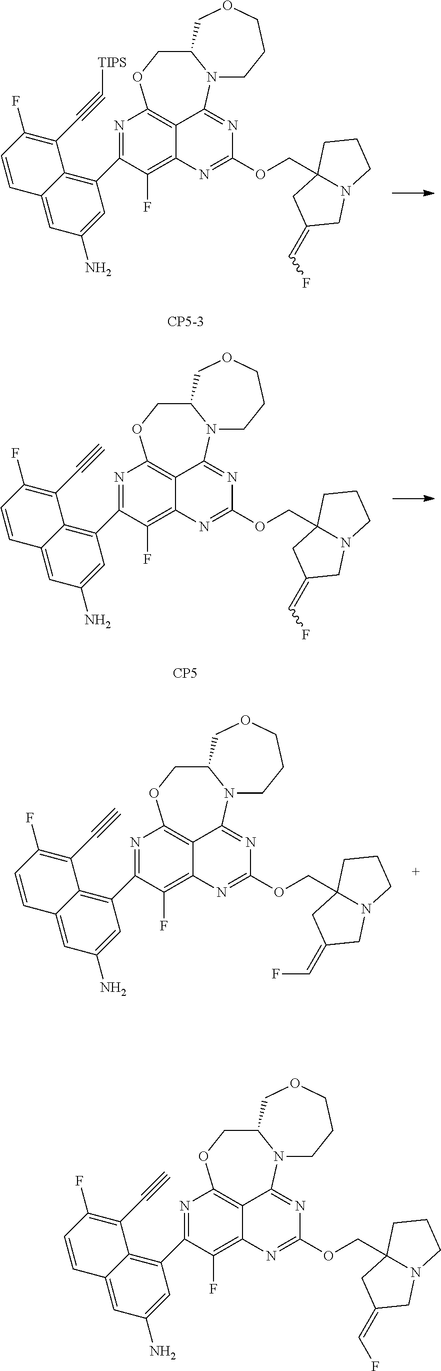

Compound 5(CP, confirmed) was synthesized in a manner similar to CP3 with 5-a and CP1-3, purified and separated by Prep-HPLC (Ultimate XB-C18, 30 mm×150 mm, Sumi, A: 0.1% TEA in water, B: CH₃CN, Gradient: 15%/B to 35% B in 30 min at a flow rate of 40 mL/min, 250 nm) to give Compound 5A (CP5A, 2.9 mg, the first peak, TFA salt) and Compound 5B (CP5B (3.6 mg, the second peak, TFA salt). MS (ESI, m/z): 629[M+H]⁺.

### Example 6

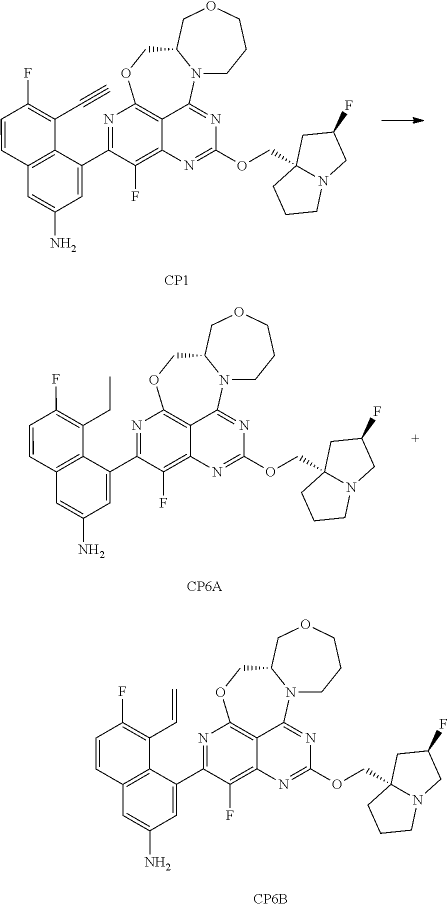 [SMILES: C#Cc1c(F)ccc2cc(N)cc(-c3nc4c5c(nc(OC[C@@]67CCCN6C[C@H](F)C7)nc5c3F)N3CCCOC[C@H]3CO4)c12 ; "CP1" ; CCc1c(F)ccc2cc(N)cc(-c3nc4c5c(nc(OC[C@@]67CCCN6C[C@H](F)C7)nc5c3F)N3CCCOC[C@H]3CO4)c12 ; "CP6A" ; C=Cc1c(F)ccc2cc(N)cc(-c3nc4c5c(nc(OC[C@@]67CCCN6C[C@H](F)C7)nc5c3F)N3CCCOC[C@H]3CO4)c12 ; "CP6B"]

To a solution of CP1 (30 mg) in MeOH (10 mL) was added Pd/C (75 mg, 10% content). The mixture was stirred for 1.5 hours at RT under H₂, filtered and the filtrate was concentrated, purified and separated by Prep-HPLC (Agela Durashell C18, 30 mm×250 mm, 10 um, A: 0.1% TFA in water, B: CH₃CN, Gradient: 10% B to 29% B in 31 min at a flow rate of 40 ml/min, 230 nm) to give Compound 6A (CP6A, 4.7 mg, TFA salt, Confirmed) LCMS: m/z: 621[M+H]⁺ and Compound 6B (CP6B, 10 mg, TFA salt, Confirmed). LCMS: m/z: 619[M+H]⁺.

### Example 7

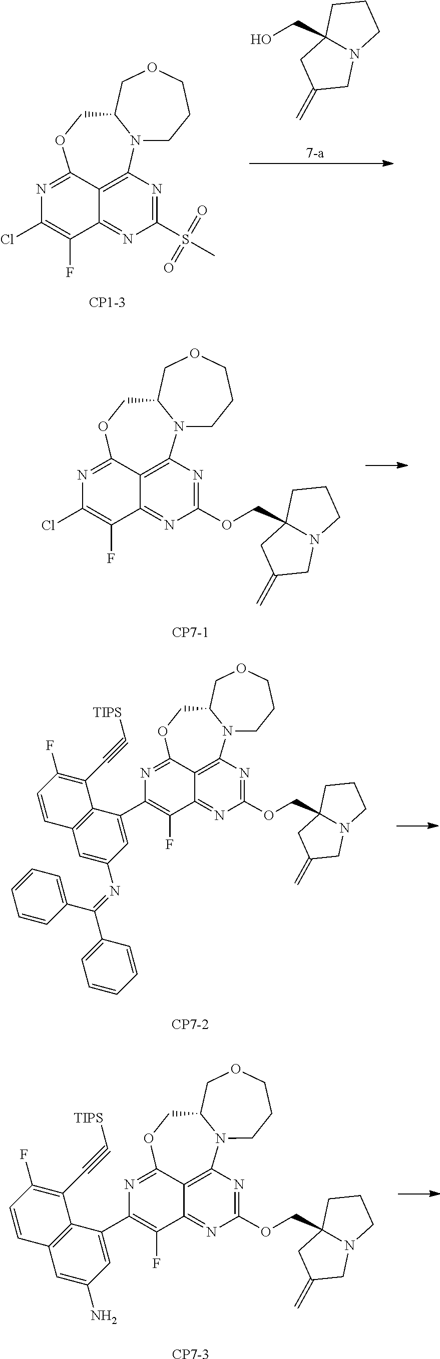

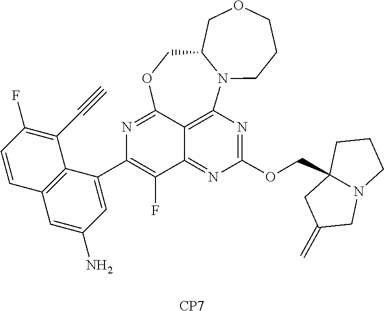

Compound 7 (CP7, 25.9 mg, TEA salt, confirmed) was synthesized in a manner similar to CP1 with 7-a and CP1-3. LCMS: m/z: 611[M+H]⁺.

### Example 8

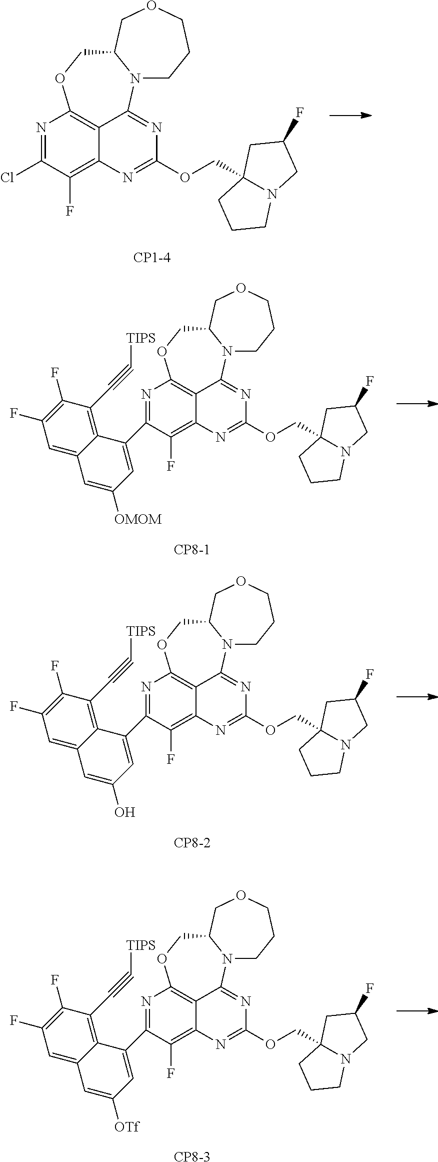

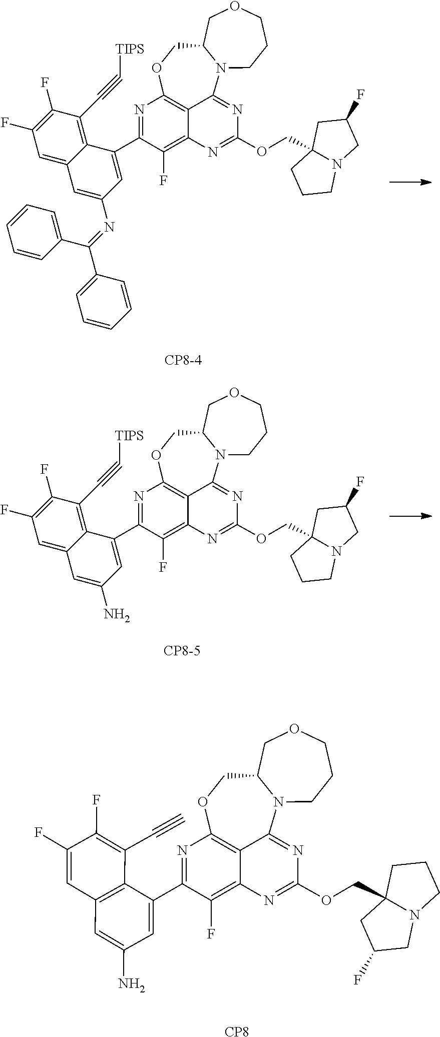

Compound 8 (CP8, confirmed) was synthesized in a manner similar to CP1 with INT11 and CP1-4. LCMS: m/z: 635[M+H]⁺.

### Example 9

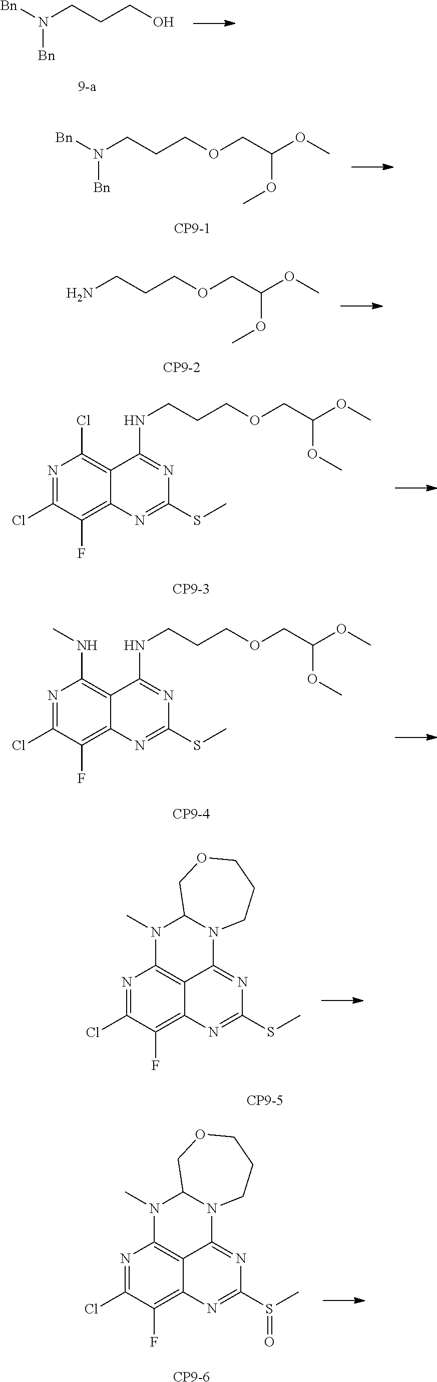

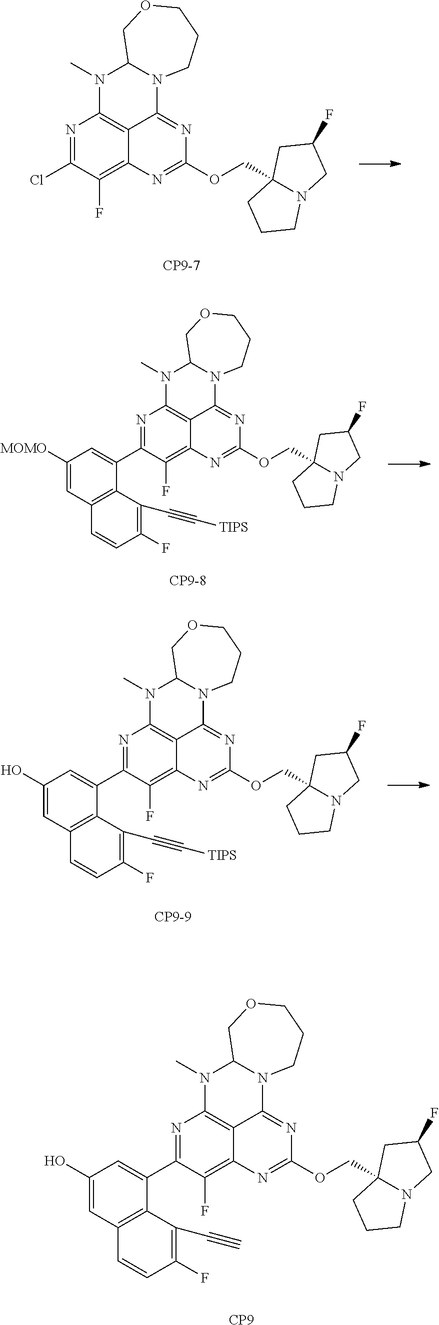

To a solution of 9-a (2.49 g) in THF (10 mL) was added NaH (60% in oil, 1.15 g) at 0° C. After stirring for 0.75h, to the mixture were added DMAP (63 mg), TBAI (394 mg) and bromoacetaldehyde dimethyl acetal (4.35 g). The reaction mixture was stirred at 70° C. for 23 hours, quenched with water and extracted with EA. The organic layer was washed with brine, dried, filtered and concentrated. The residue was purified by silica gel column to give CP9-1 (3.55 g). LCMS: m/z: 343[M+H]⁺.

To a solution of CP9-4 (1.56 g) in MeOH (25 mL) was added Pd(OH)₂/C (0.84 g). The reaction mixture was stirred at RT for 5 hours under H₂, filtered and the filtrate was concentrated to give CP9-2 (0.75 g). MS: m/z: 164[M+H]⁺.

To a solution of INT6 (1.27 g) and DIEA (1.76 g) in toluene (15 mL) was added POCl₃ (0.8 ml). The reaction mixture was stirred at 80° C. for 1.5 h and concentrated. The obtained residue was dissolved in DCM (10 mL), CP9-2 (0.75 g) and DIEA (2 ml) were added at 0° C. The reaction was stirred at RT for 1.5 h, diluted with water and extracted with EA. The collected organic layer was washed with brine, dried, concentrated and purified by silica gel column to give CP9-3 (1.38 g). MS: m/z: 425[M+H]⁺.

A mixture of CP9-3 (0.31 g), methylamine hydrochloride (216 mg), Cs₂CO₃ (1254 mg) and DMAc (4 mL) in 8 ml sealed bottle was stirred at 100° C. for 17 h, diluted with water and extracted with EA. The organic layer was washed with brine, dried, concentrated and purified by Prep-TLC to give CP9-4 (226 mg). MS: m/z: 420[M+H]⁺.

To a solution of CP94 (200 mg) in 1,4-dioxane (4 mL) was added TsOH·H₂O (97 mg). The solution was stirred at 100° C. for 24 h under N₂, diluted with water and extracted with EA. The collected organic layer was washed with brine, dried, concentrated and purified by Prep-TLC to give CP9-5 (52 mg). MS: m/z: 356[M+H]⁺.

To a solution of CP9-5 (52 mg) in DCM (10 mL) was added m-CPBA (50 mg) at RT. The mixture was stirred for 0.75 h, quenched with aq. Na₂S₂O₃ and extracted with DCM. The organic layer was washed with sat. aq. NaHCO₃, dried and concentrated to give CP9-6 (58 mg). MS: m/z: 372[M+H]⁺.

To a solution of INT14 (42 mg) in THF (3 mL) was added t-BuONa (30 mg). After stirring for 10 min, CP9-6 (58 mg) in THF (1 ml) was added. The mixture was stirred at RT for 0.5 h, quenched with water and extracted with EA. The organic layer was washed with brine, dried, concentrated and was purified by Prep-TLC to give CP9-7 (53 mg). MS: m/z: 467[M+H]⁺.

To a solution of CP9-7 (53 mg), INT2 (77 mg) in toluene (6 mL) and water (1.5 mL) were added cataCXium A Pd G3 (23 mg) and Cs₂CO₃ (91 mg). The reaction mixture was stirred at 100° C. for 18 hours under N₂. The mixture was diluted with water and extracted with EA. The collected organic layer was washed with aq.NaCl, dried, concentrated and purified by Prep-TLC to give CP9-8 (43 mg). MS: m/z: 817[M+H]⁺.

A solution of CP9-8 (43 mg), HCl (4M in dioxane, 1 mL) in DCM (3 mL) was stirred at RT for 1 h. The solution was concentrated, diluted with sat.NaHCO₃ solution, extracted with EA. The collected organic layer was washed with brine, dried and concentrated to give CP9-9 (51 mg, crude). MS: m/z: 773[M+H]⁺.

A mixture of CP9-9 (51 mg, crude) and CsF (0.30 g) in DMF (5 mL) was stirred at 40° C. for 1.5 h. The mixture was diluted with water and extracted with EA. The organic layer was dried, concentrated and purified by Prep-HPLC (Daisogel-C18, 50 mm×250 mm, 10 um, A: 0.05% TFA in water, B: CH₃CN, Gradient: 20% B to 45% B in 35 min at a flow rate of 70 mL/min, 225 nm) to freeze-dried to give Compound 9 (CP9, 8 mg, TFA salt, confirmed). MS: m/z: 617[M+H]⁺.

### Example 10

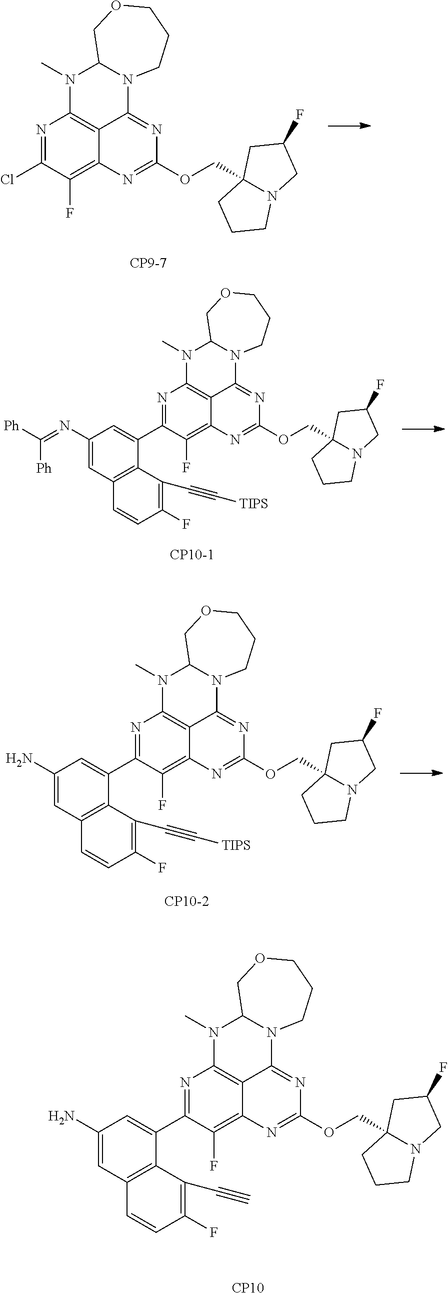

Compound 10 (CP10, confirmed) was synthesized in a manner similar to CP9 with CP9-7 and INT13 and purified by Prep-HPLC (YMC-Triart C18-S12 nm, phase A: 0.1% TFA in water, phase B: CH₃CN, Gradient: 15% B to 45% B in 30 min at a flow rate of 70 mL/min, 226 nm). LCMS: m/z: 616[M+H]⁺.

### Example 11

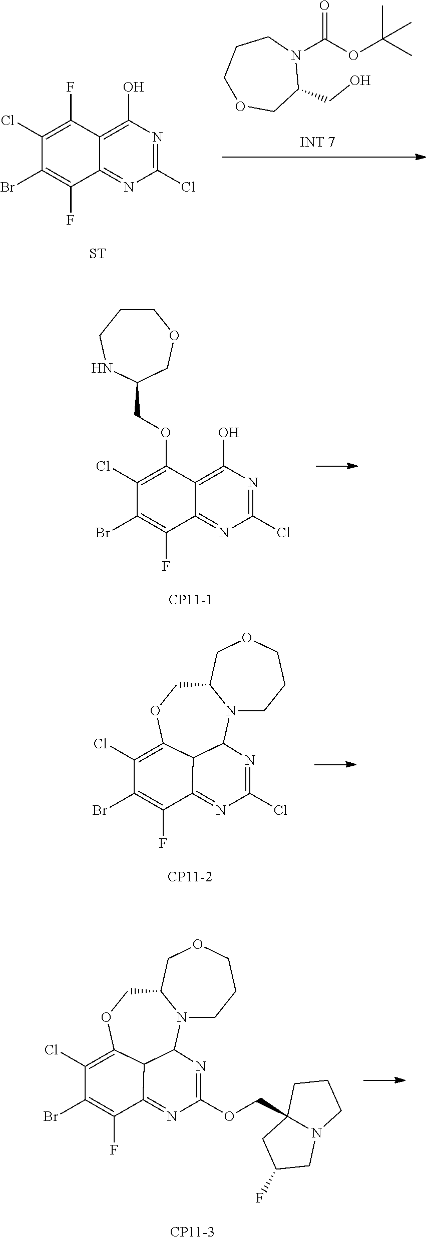

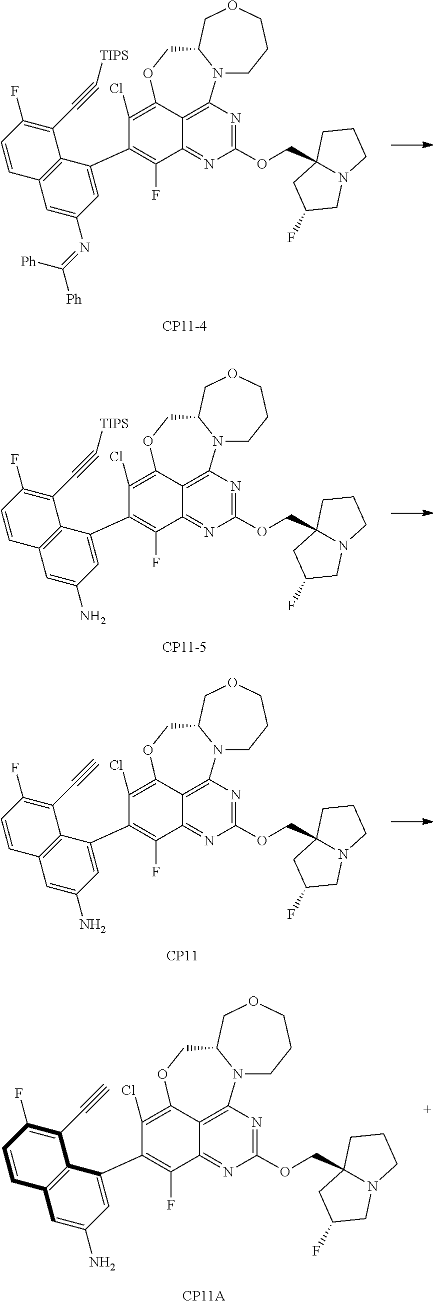

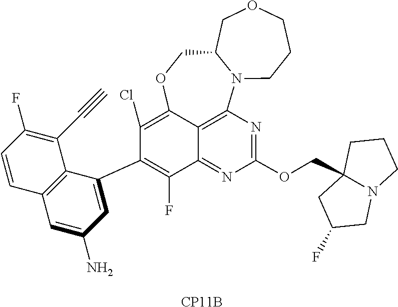

CP11-3 was synthesized in a manner similar to CP1-4 with INT7 and ST.

Compound 11(CP11, 7.1 mg) was synthesized in a manner similar to CP3 with CP11-3 and INT13. MS: m/z: 650[M+H]⁺.

CP11 (3.08 mg) was separated by Prep-HPLC Gilson with the conditions: Column, CHIRAL ART Cellulose-SC column (2 cm×25 cm, 5 um); mobile phase, (Hex:DCM=3:1)(0.1% DEA)/EtOH (50:50); Flowing rate: 17 ml/min. This resulted in the first peak Compound 11A (CP11A, 2.8 mg, Retention time: 5.510 min, confirmed) and Compound 11B (CP11B, 2.4 mg, the second peak, Retention time: 8.137 min, confirmed).

### Example 12

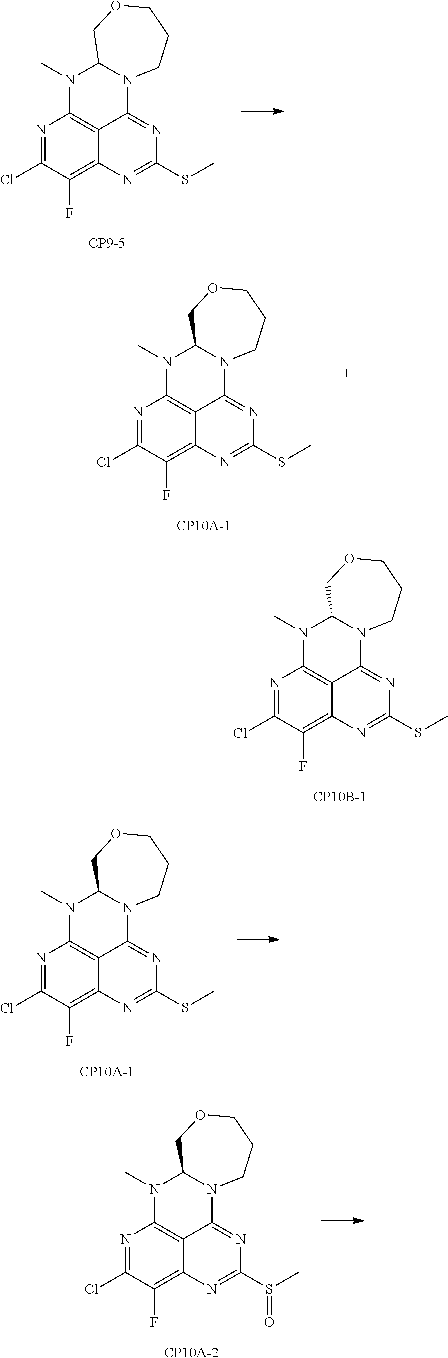

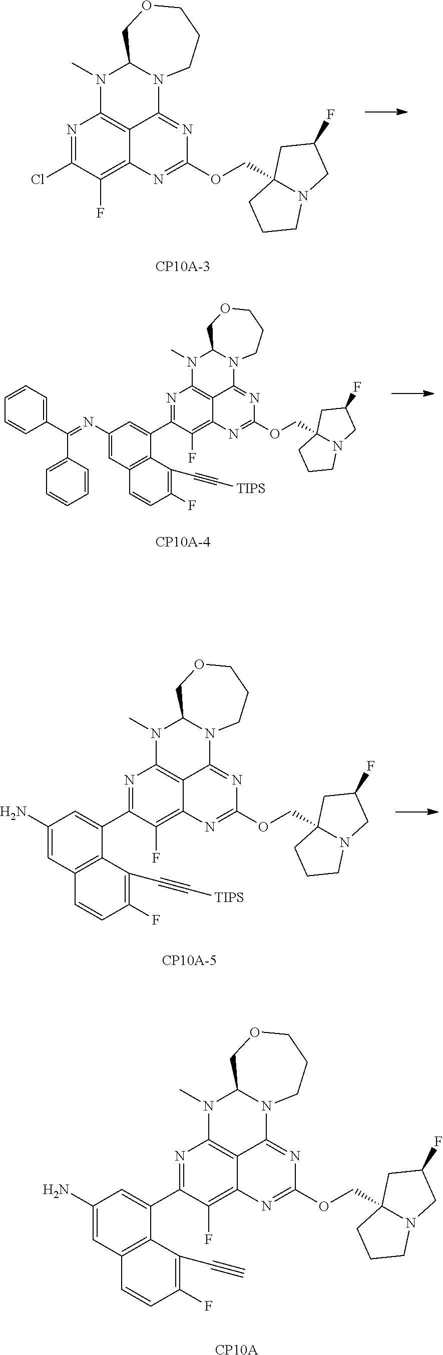

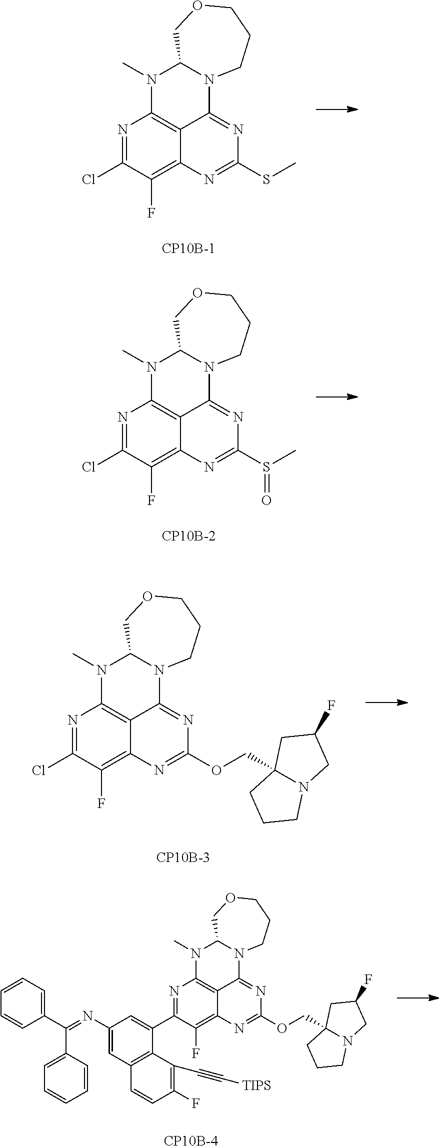

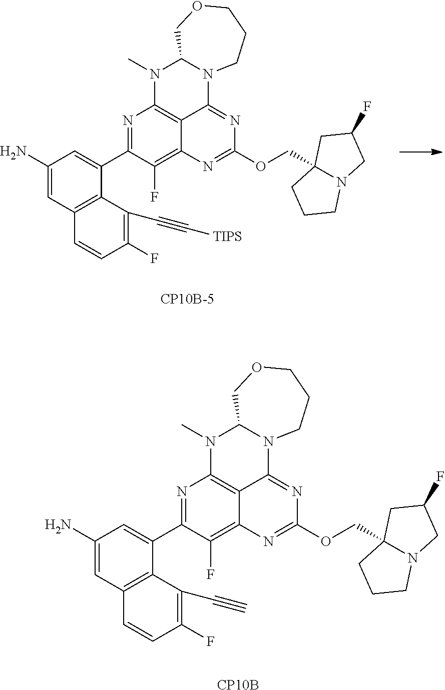

CP9-5 (0.155 g) was separated by Prep-HPLC-Gilson with the following conditions: Column. CHIRAL ART Cellulose-SC column (2 cm×25 cm, 5 um); mobile phase. (Hex:DCM=3:1)(0.1% DEA)/EtOH (50:50); Flowing rate: 16 mL/min to afford CP10A-1 (83 mg. Retention Time: 6.65 min) and CP10B-1 (61 mg, Retention Time: 9.253 min) respectively.

CP10A-3 was synthesized in a manner similar to CP9-7 with CP10A-1.

Compound 10A (CP10A, confirmed) was synthesized in a manner similar to CP3 with CP10A-3, purified by Prep-HPLC (Daisogel-C18, phase A: 0.1% TFA in water, phase B: CH₃CN, gradient: 15% B to 36% B in 38 min at a flow rate of 60 mL/min, 230 nm) and freeze-dried to afford CP10A (26.4 mg, TFA salt). MS (ESI, m/z): 616[M+H]⁺.

Compound 10B (CP10B, confirmed) was synthesized in a manner similar to CP10A with CP10B-1, purified by Prep-HPLC (Daisogel-C18, phase A: 0.1% TFA in water, phase B: CH₃CN, gradient: 15% B to 35% B in 32 min at a flow rate of 60 mL/min, 230 nm) and freeze-dried to afford CP10B (12.3 mg, TFA salt). MS (ESI, m/z): 616[M+H]⁺.

### Example 13

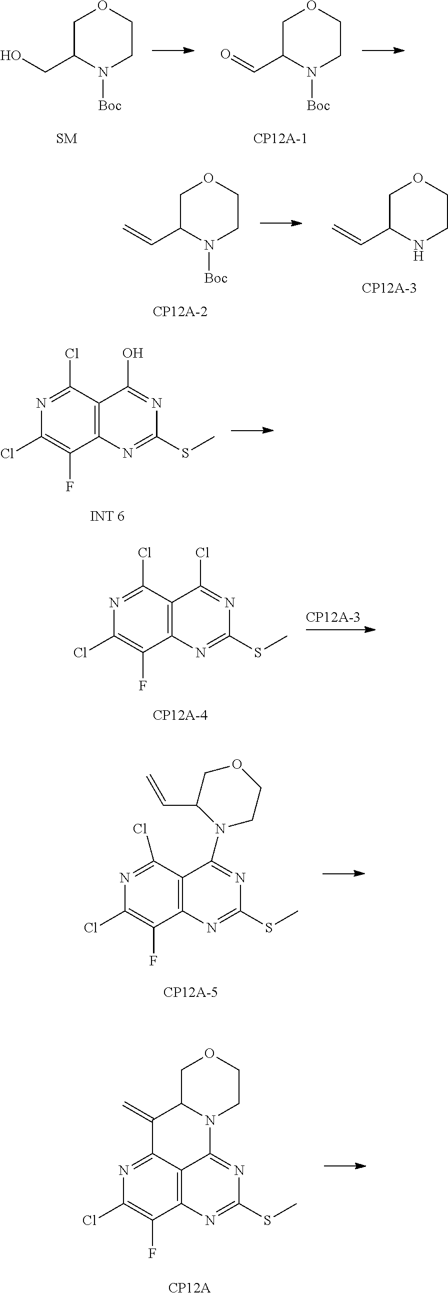

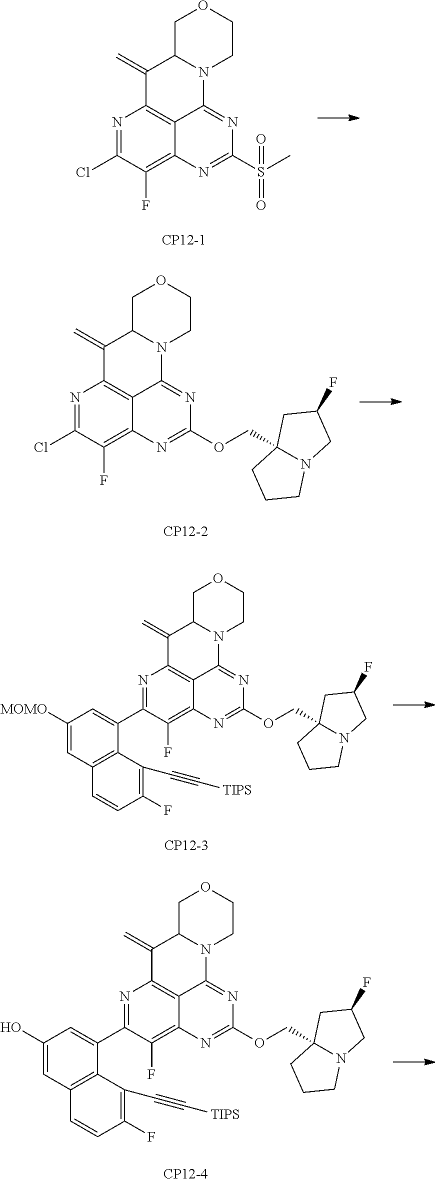

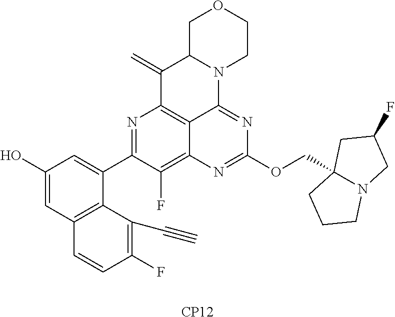

To a solution of SM (2.6 g) in DCM (80 mL) was added Dess-Martin (6.1 g) at rt. the reaction mixture was stirred overnight, quenched with 5% sodium thiosulphate in saturated NaHCO₃ aqueous solution. The resulting biphasic mixture was stirred vigorously for 15 minutes and washed with DCM. The separated organic layer was post processed to afford CP12A-1 (1.0 g). LCMS: m/z: 216[M+H]⁺.

A solution of methyltriphenylphosphonium iodide (3.0 g) and t-BuOK (834.6 mg) in THF (37 mL) at RT was heated to 45° C. and stirred for 1 h. CP12A-1 (800 mg) was added and stirred at RT overnight. The mixture was poured into water and extracted with EA. The separated organic layer was post processed to afford CP12A-2 (350 mg). LCMS: m/z: 214[M+H]⁺.

To a solution of CP12A-2 (40 mg) in dioxane (1.6 mL) was added 4M HCl/dioxane (0.4 mL) at 0° C. and the resulting mixture was stirred for 1 h. the reaction mixture was concentrated to afford CP12A-3 (crude) without any purification. MS (ESI, m/z): 114.3[M+H]⁺.

To a solution of INT6 (52 mg) in POCl₃ (1 mL) was added DIEA (0.1 mL) and the resulting mixture was stirred at 110° C. for 1 h. the reaction mixture was concentrated to afford CP12A-4 (crude) without any purification.

To a solution of CP12A-4 (crude, 0.188 mmol) in DCM (2 mL) at −40° C. was added DIEA (162.4 mg) and CP12A-3 (crude, 0.188 mmol). The reaction mixture was stirred at −40° C. for 1 h. Upon completion. H₂O was added to the reaction mixture and extracted with DCM. The separated organic layer was post processed to give CP12A-5 (30 mg). LCMS: m/z: 375[M+H]⁺.

To a solution of CP12A-5 (30 mg), K₃PO₄ (50.9 mg) and Pd(dppf)Cl₂ (6 mg) in toluene/H₂O=10/1 (1.1 mL) were added at RT. The reaction mixture was stirred at 105° C. overnight under N₂. Upon completion. H₂O was added to the reaction mixture and extracted with DCM. The separated organic layer was post processed to give CP12A (10 mg) as a yellow solid. MS (ESI, m/z): 339[M+H]⁺. ¹HNMR (300 MHz, DMSO-d₆): δ6.63 (s, 1H), 5.46 (s, 1H), 5.04-4.99 (m, 1H), 4.75-4.70 (m, 1H), 4.04-3.97 (m, 2H), 3.66-3.54 (m, 2H), 3.25-3.21 (m, 1H), 2.61 (s, 3H).

CP12-2 was synthesized in a manner similar to CP1-4 with CP12A.

Compound 12 (CP12, 1.4 mg, confirmed) was synthesized in a manner similar to CP2 with CP12-2. LCMS: m/z: 600[M+H]⁺.

### Example 14

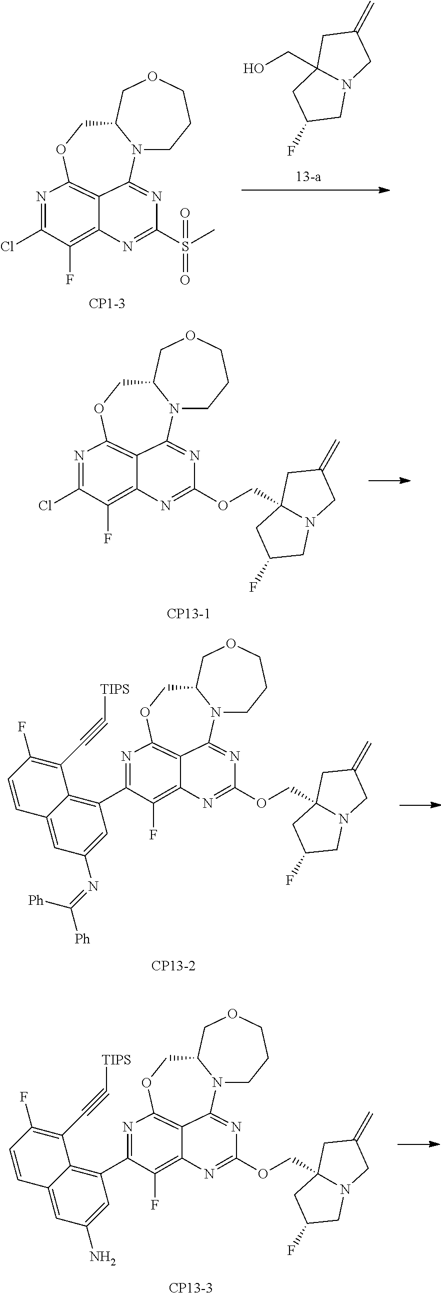

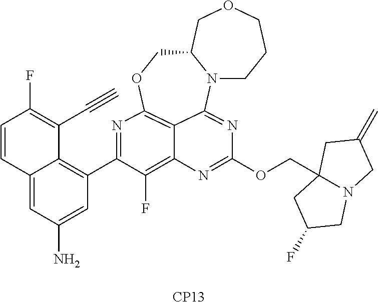

Compound 13 (CP13, 8 mg, TFA salt, confirmed) was synthesized in a manner similar to CP5 using CP1-3 and 13-a. LCMS: m/z: 629[M+H]⁺.

### Example 15

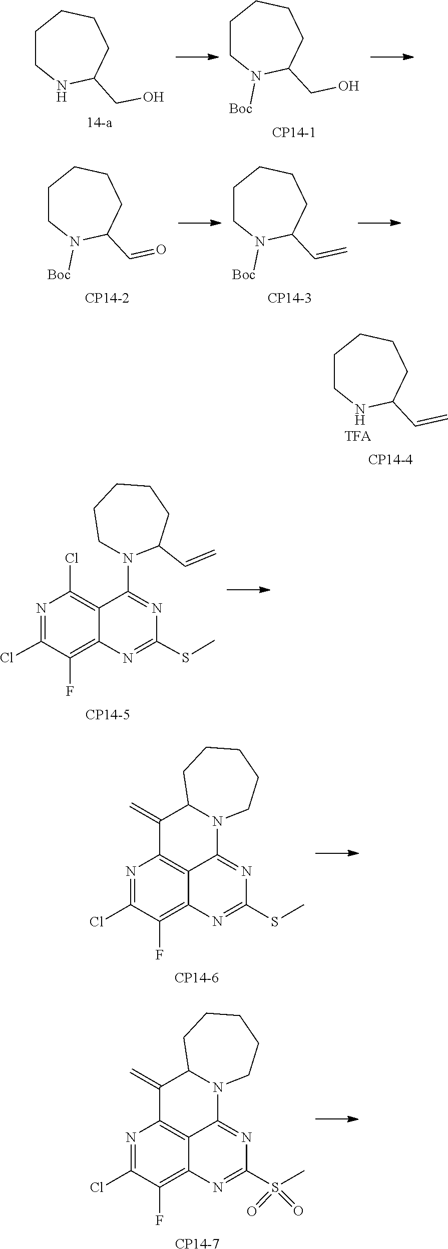

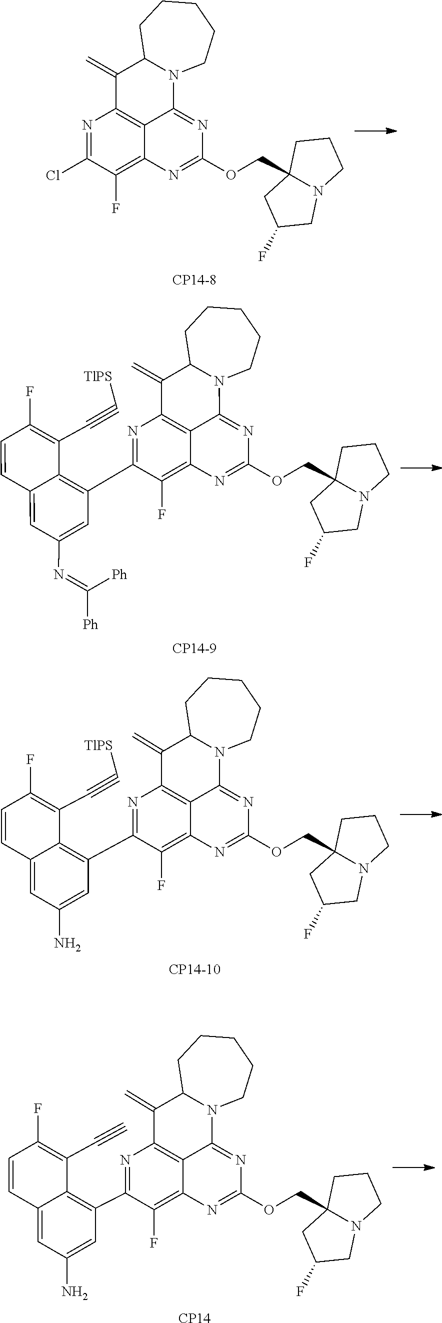

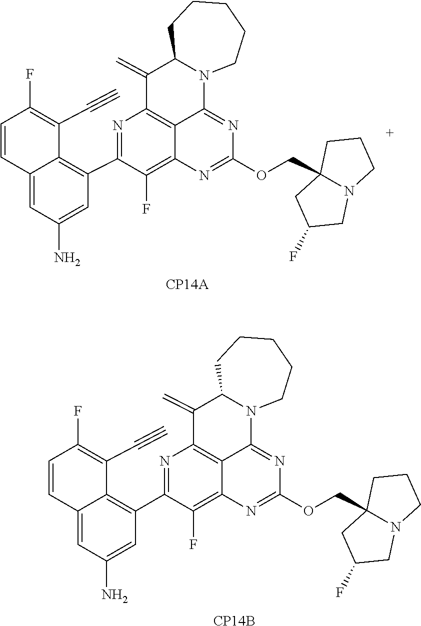

A solution of 14-a (1.119 g), TEA (3.408 g) and Boc₂O (2.483 g) in DCM (30 mL) was stirred for 16 h at RT. The reaction was diluted with DCM and washed with water. The separated organic layer was post processed to afford CP14-1 (1542 mg). LCMS: m/z: 230[M+H]⁺.

CP14-8 (321 mg) was synthesized in a manner similar to CP12-2 with CP14-1.

Compound 14 (CP14) was synthesized in a manner similar to CP3 with CP14-8 and separated by Prep-HPLC (C18 column, A: 0.05% TFA in water, B: CH₃CN, gradient: 25% B to 45% B in 40 min; at a flow rate of 70 mL/min, 230 nm) to give Compound 14A (CP14A, 12 mg, the first peak, confirmed) and Compound 14B (CP14B, 42 mg, the second peak, confirmed). LCMS: m/z: 611 [M+H]⁺.

### Example 16

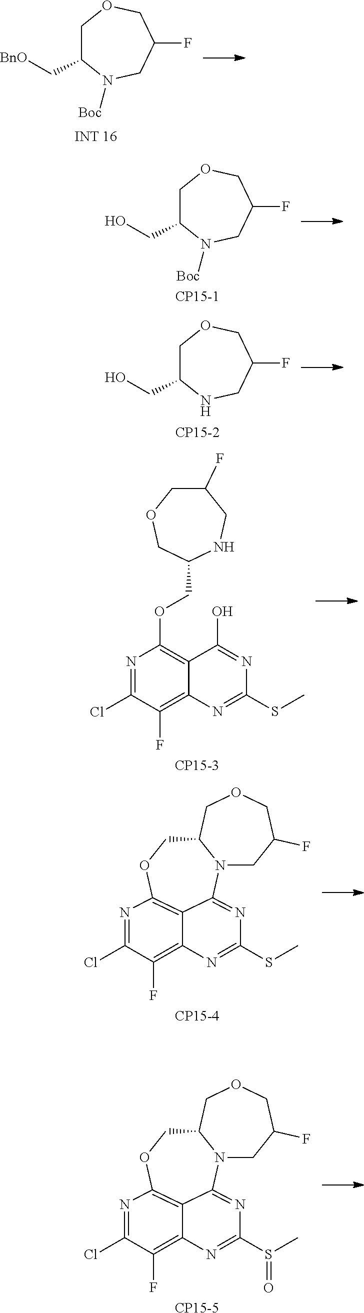

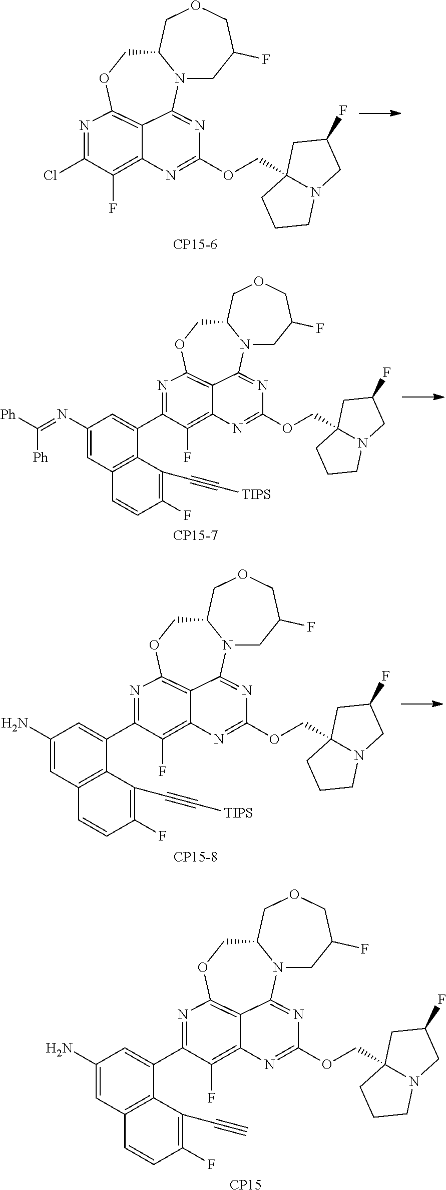

Compound 15 (CP15, confirmed) was synthesized in a manner similar to CP30 with INT16.

### Example 17

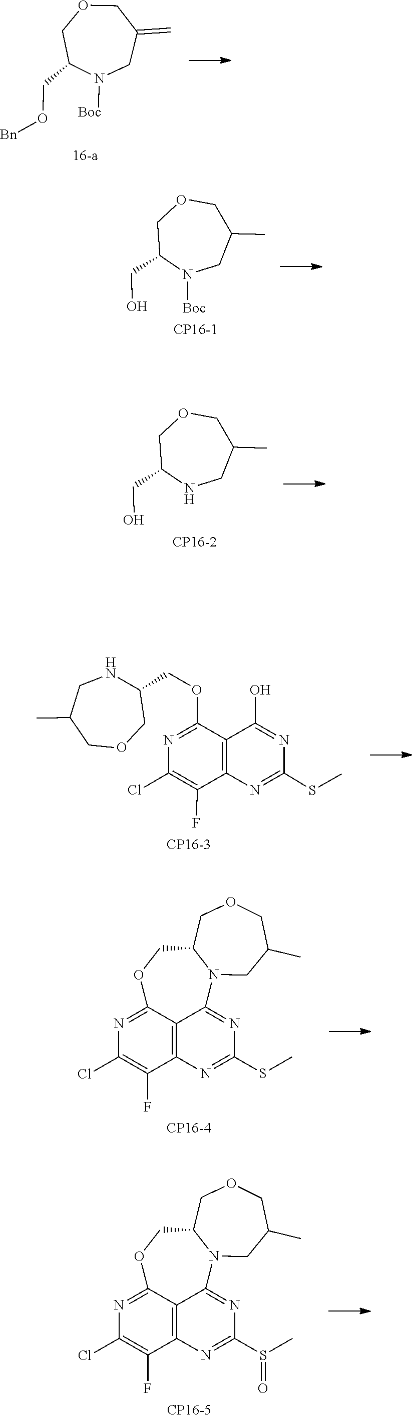

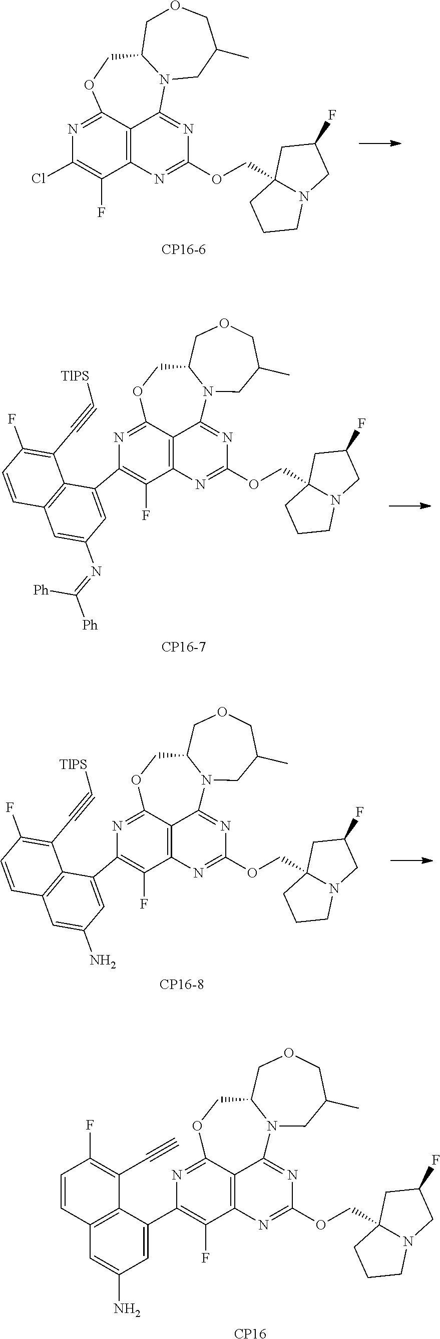

To a solution of 16-a (249 mg) in methanol (15 mL) was added Pd/C (235 mg). The solution was stirred under H₂ for 6 h, filtered and the filtrate was concentrated to afford CP16-1 (201 mg). MS (ESI, m/z): 246 [M+H]⁺.

To a solution of CP16-1 (201 mg) in CH₃CN (6 mL) was added HCl (4M in 1,4-dioxane, 2 mL). The reaction mixture was stirred at RT for 1.5 hour and concentrated. The residue was added CH₃CN (5 mL) and aq. NaHCO₃ (1 mL), ultrasound and concentrated. The residue was added DCM and filtered. The filtrate was concentrated to afford CP16-2 (168 mg). MS (ESI, m/z): 146[M+H]⁺.

A solution of CP16-2 (168 mg) in THF (10 mL) was cooled down by ice ethanol bath. NaH (194 mg, 601% content in oil) was added in batches, followed by INT6 (331 mg). The reaction mixture was stirred for 2 h, quenched with 2 drops of water. The solution was purified with RP-flash to afford CP16-3 (161 mg). MS (ESI, m/z): 389[M+H]⁺.

To a solution of CP16-3 (161 mg) in DCM (15 mL) was added DIEA (326 mg) and BOP—Cl (362 mg). The reaction mixture was stirred at RT for 24 hours, quenched with water (30 mL) and extracted with DCM (30 mL). The separated organic layer was post processed to afford CP16-4 (30 mg). MS (ESI, m/z): 371[M+H]⁺.

CP16-6 was synthesized in a manner similar to CP1-4 with CP16-4.

Compound 16 (CP16, 1.2 mg, TFA salt, confirmed) was synthesized in a manner similar to CP3 with CP16-6. LCMS: m/z: 631[M+H]⁺.

### Example 18

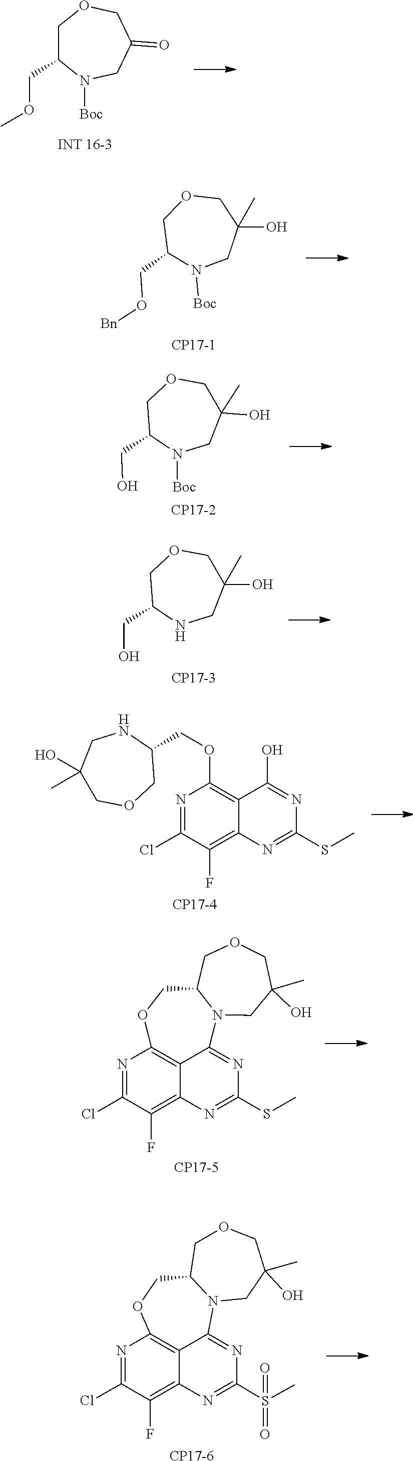

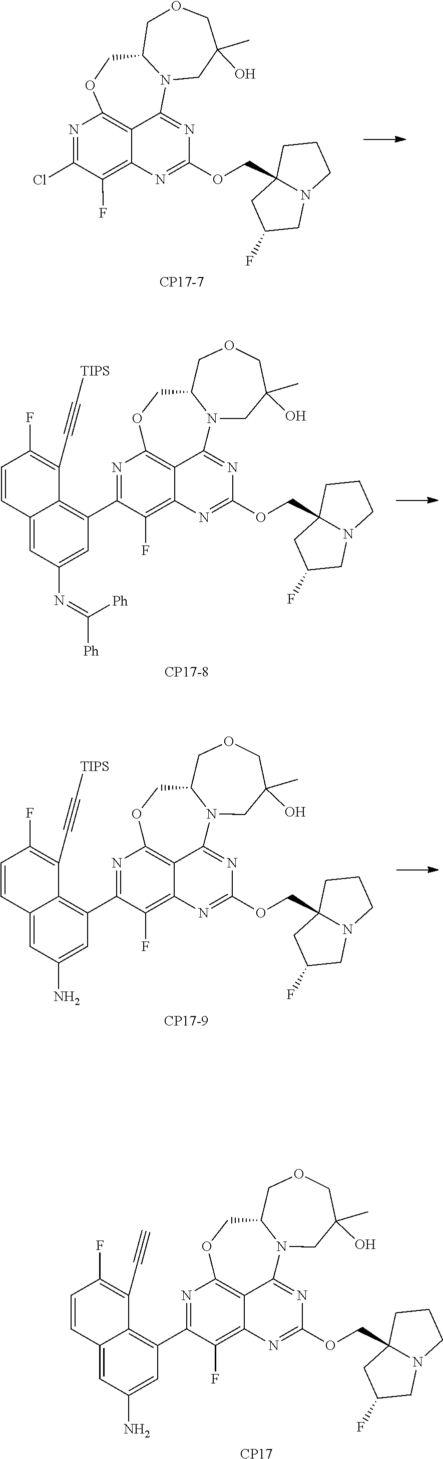

A solution of INT16-3 (352 mg) in THF (10 mL) was cooled to −70° C. under N₂. Methylmagnesium bromide (1M in THF, 4 mL) was added dropwise and the resulting mixture was stirred for 1 h at −70° C., quenched by water and extracted with EA. The separated organic layer was post processed to afford CP17-1 (361 mg). MS (ESI, m/z): 352[M+H]⁺.

To a solution of CP17-1 (361 mg) in methanol (15 mL) was added Pd/C (354 mg) at H₂. The reaction mixture was stirred at rt for 16 hours, filtered and the filtrate was concentrated to afford CP17-2 (236 mg). MS (ESI, m/z): 262[M+H]⁺.

CP17-7 was synthesized in a manner similar to CP16-6 with CP17-2.

Compound 17 (CP17, 19.1 mg, TFA salt, confirmed) was synthesized in a manner similar to CP3 with CP17-7. LCMS: m/z: 647[M+H]⁺.

### Example 19

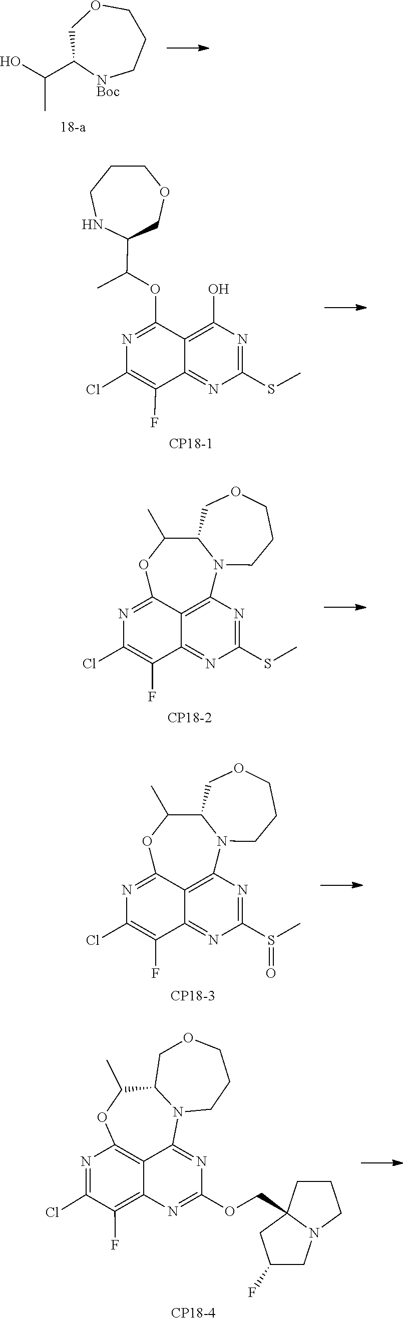

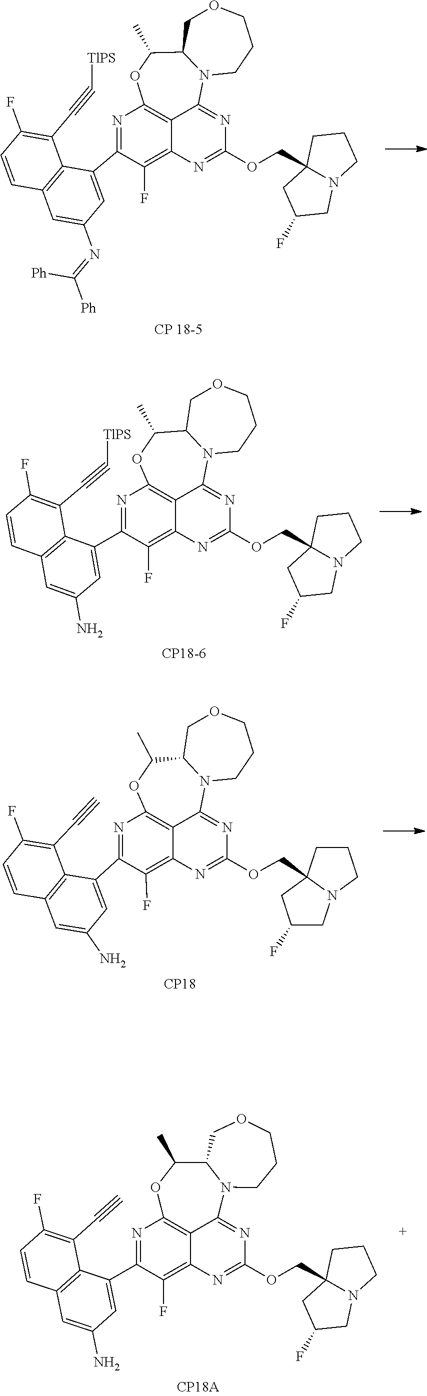

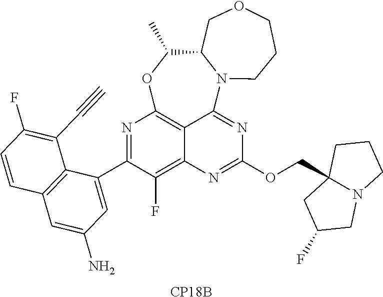

CP18-6 was synthesized in a manner similar to CP16-8 with 18-a.

To a solution of CP18-6 (153 mg) in DMF (5 mL) was added CsF (466 mg). The reaction mixture was stirred at 40° C. for 4 h. The reaction was added EA (40 mL) and washed with aq. NaHCO₃ (30 mL). The separated organic layer was post processed and separated by Prep-HPLC (YMC-Triart C18-S12 nm, 50 mm×250 mm, 7 um, phase A: 0.1% TFA in water, phase B: CH₃CN, gradient: 15% B to 43% B in 35 min at a flow rate of 70 mL/min, 220 nm) and freeze-dried to afford Compound 18A (CP18A, the first peak, 70.9 mg, TFA salt, confirmed), MS (ESI, m/z): 631[M+H]⁺ and Compound 18B (CP18B, the second peak, 20.4 mg, TFA salt, confirmed), MS (ESI, m/z): 631 [M+H]⁺.

### Example 20

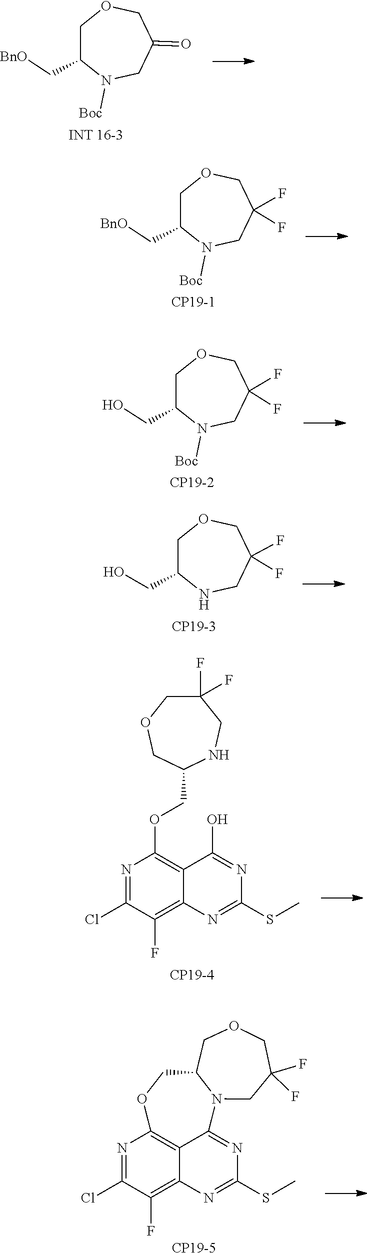

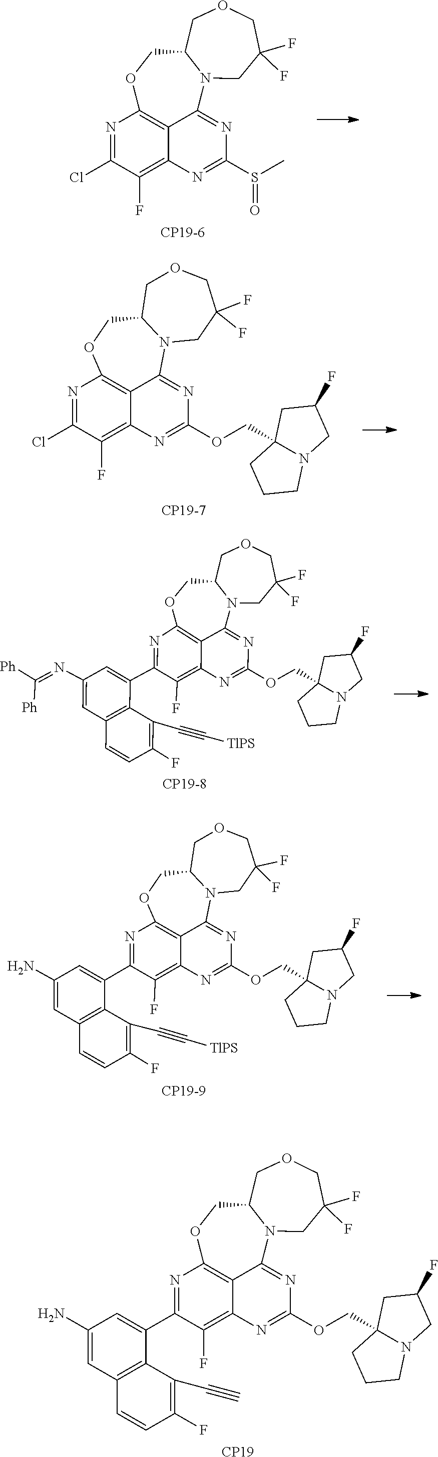

To a solution of INT16-3 (821 mg) in DCM (20 mL) was added DAST (2.38 g) at ice ethanol bath and stirred for 1.5 h. the solution was warmed to RT and stirred for 5 h. the mixture was diluted with DCM (30 mL) and washed with aq. NaHCO₃ (50 mL). The separated organic layer was post processed to afford CP19-1 (565 mg). MS (ESI, m/z): 358[M+H]⁺.

To a solution of CP19-1 (565 mg) in methanol (15 mL) was added Pd(OH)₂/C (409 mg) at H₂. The reaction mixture was stirred at RT for 21 hours, filtered and the filtrate was concentrated to afford CP19-2 (439 mg). MS (ESI, m/z): 268[M+H]⁺.

Compound 19 (CP19, 39 mg, confirmed) was synthesized in a manner similar to CP16 with CP19-2. LCMS: m/z: 653[M+H]⁺.

### Example 21

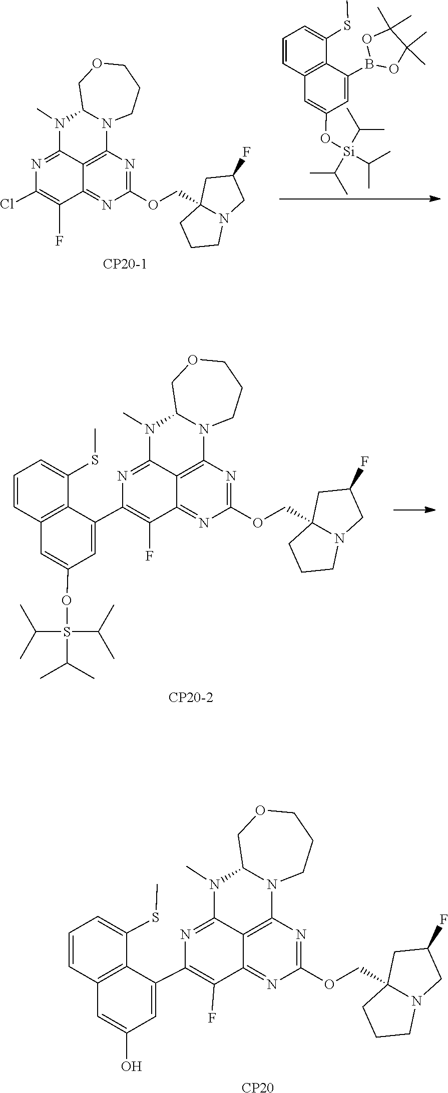

Compound 20 (CP20, confirmed) was synthesized in a manner similar to CP9.

### Example 22

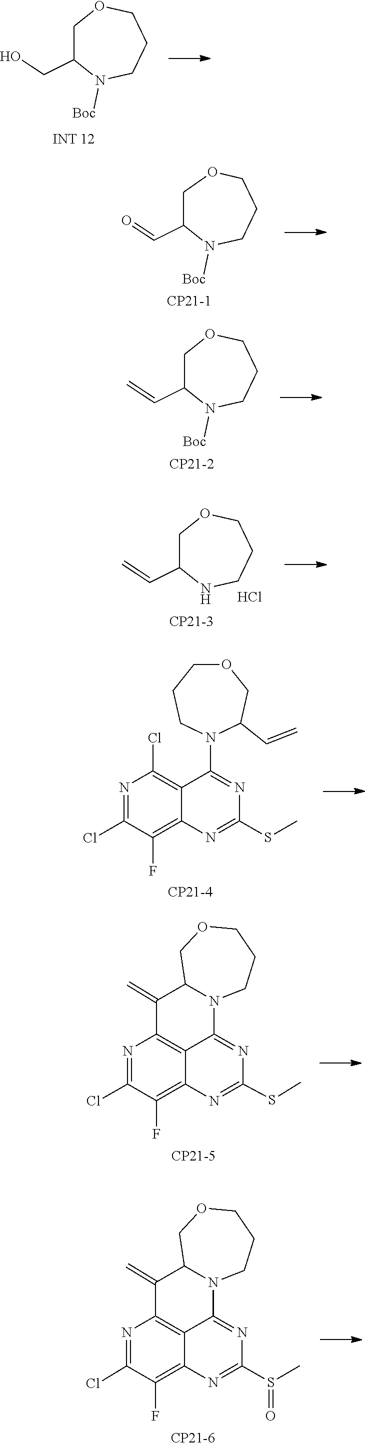

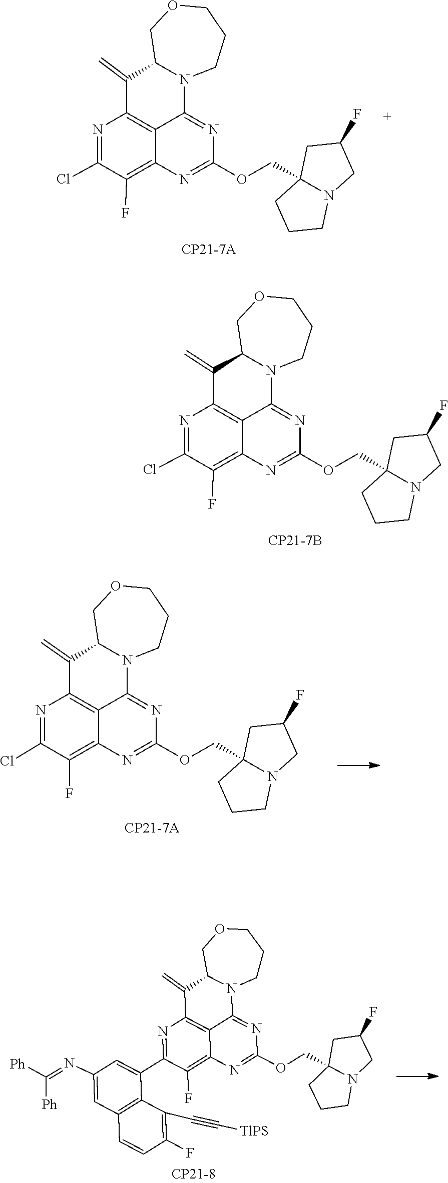

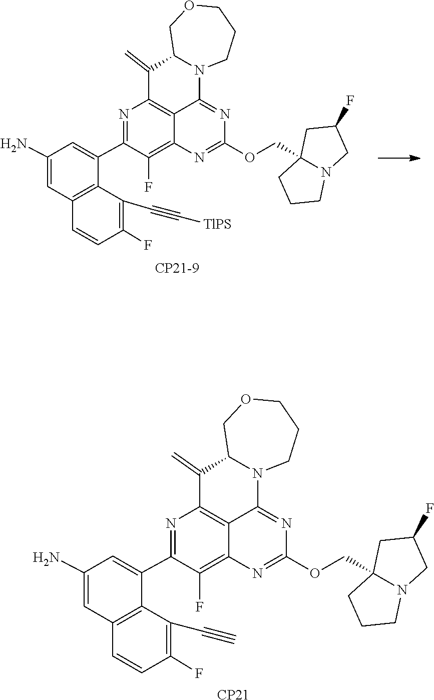

CP21-7A/21-7B was synthesized in a manner similar to CP12-2 with INT12 and separated by Prep-TLC to give two isomers CP21-7A (45 mg) and CP21-7B (38 mg). MS: m/z: 464 [M+H]⁺.

Compound 21 (CP21, confirmed) was synthesized in a manner similar to CP3 with CP21-7A. LCMS: m/z: 613 [M+H]⁺.

### Example 23

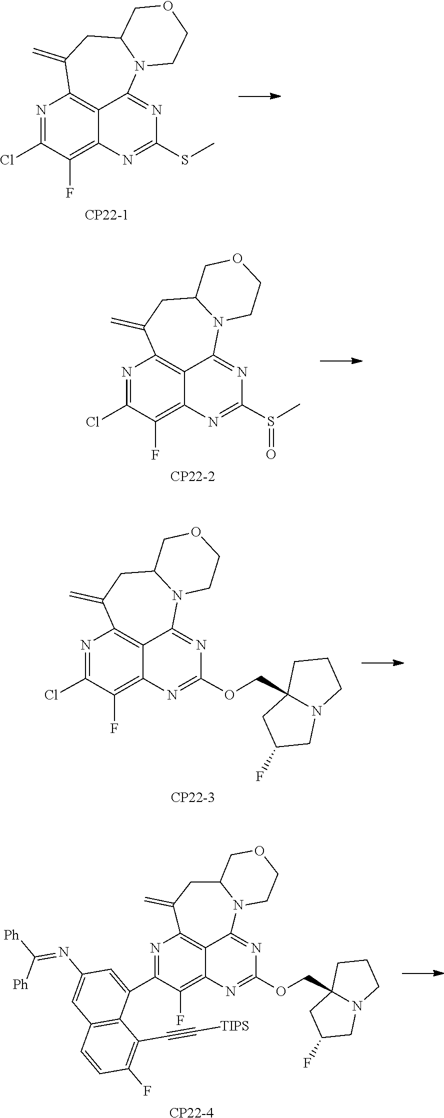

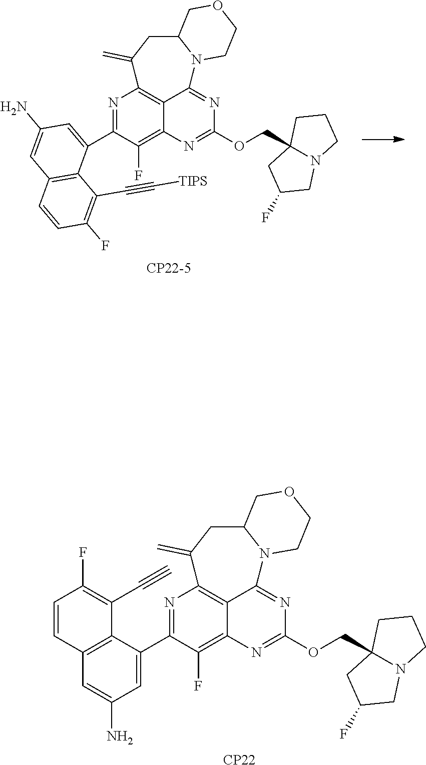

CP22-1 was synthesized in a manner similar to CP12A.

Compound 22 (CP22, 24.6 mg, TFA salt, confirmed) was synthesized in a manner similar to CP21 with CP22-1. LCMS: m/z: 613[M+H]⁺.

### Example 24

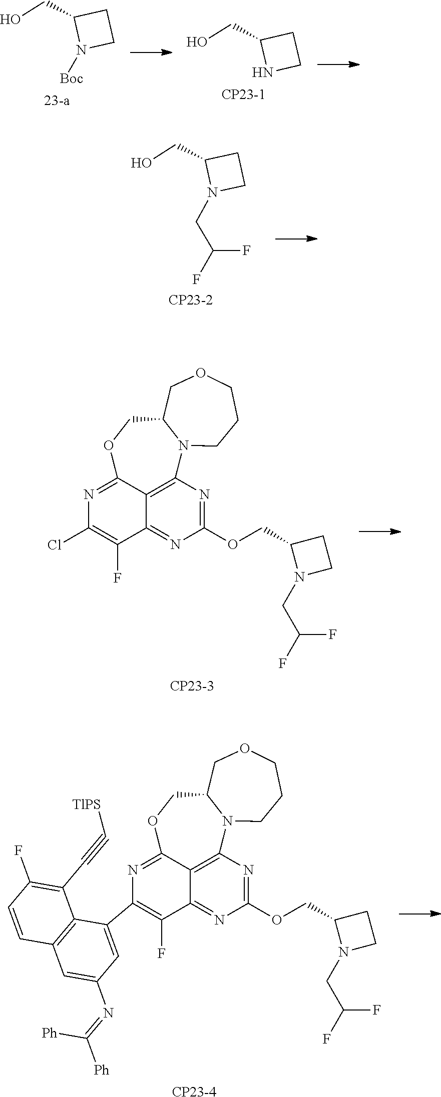

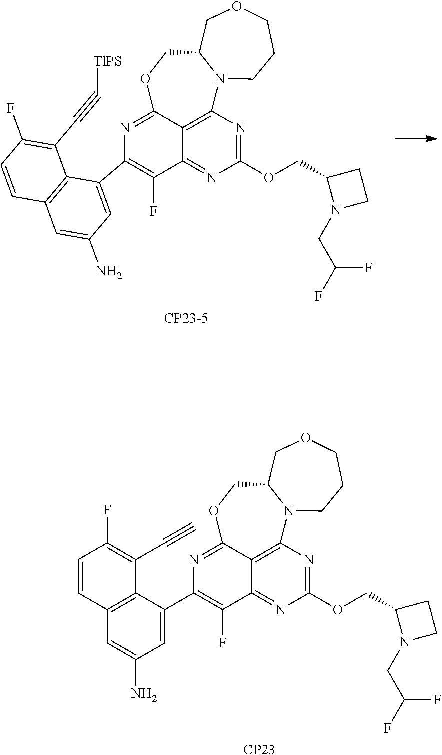

A solution of 23-a (3.02 g) in HCl (4M in 1,4-dioxane, 30 mL) was stirred at RT for 3 hours and concentrated to afford CP23-1 (2.63 g). MS (ESI, m/z): 88[M+H]⁺.

A solution of CP23-1 (1.35 g), 2,2-difluoroethyl trifluoromethanesulfonate (1.42 g) and K₂CO₃ (3.45 g) in CH₃CN (20 mL) was stirred at RT for 16 hours. The mixture was diluted with water and extracted with EA. The organic layer was post processed to afford CP23-2 (262 mg). MS (ESI, m/z): 152[M+H]⁺.

To a solution of CP1-3 (308 mg), CP23-2 (125 mg) in THE (10 mL) was added t-BuONa (127 mg). The reaction mixture was stirred for 1 hour at RT. The mixture was diluted with water and extracted with EA. The separated organic layer was post processed to afford CP23-3 (56 mg). MS (ESI, m/z): 460[M+H]⁺.

Compound 23 (CP23, 16.0 mg. TFA salt, confirmed) was synthesized in a manner similar to CP3 with CP23-3. LCMS: m/z: 609[M+H]⁺.

### Example 25

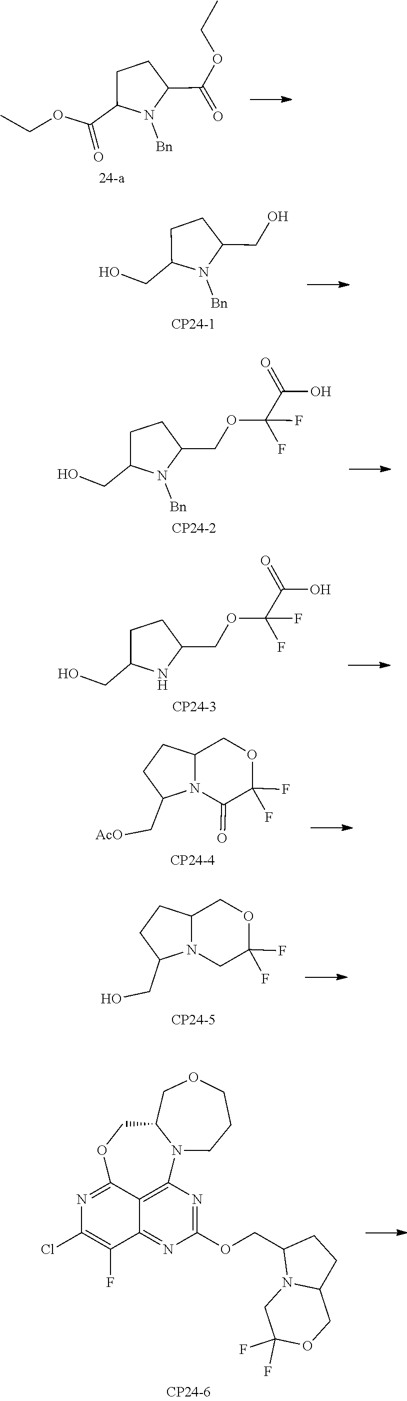

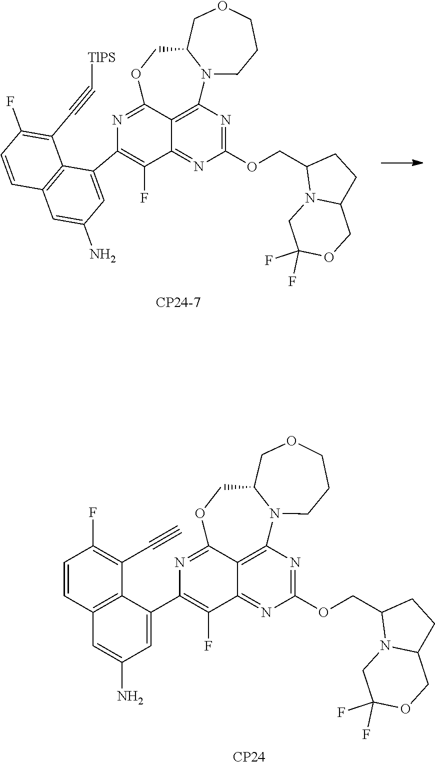

To a solution of 24-a (14.20 g) in THE (150 mL) was added LAH (2.78 g) at 0° C. The resulting mixture was stirred at RT for 1 h, quenched by water at ice water bath, filtered and extracted by EA. The separated organic layer was post processed to afford CP24-1 (11.01 g). MS (ESI, m/z): 222[M+H]⁺.

A solution of CP24-1 (1.15 g) in THE (15 mL) was cooled down to 0-5° C. at ice water bath. NaH (218 mg) was added in batches and the resulting mixture was stirred at the same temperature for 30 min. sodium bromodifluoroacetate (801 mg) was added and stirred for 16 h at RT, quenched by aq. NH₄Cl and extracted with EA. The aqueous layer was concentrated to remove most of it. The remaining part was post processed to afford CP24-2 (739.9 mg). MS (ESI, m/z): 316[M+H]⁺.

To a solution of CP24-2 (589.2 mg) in methanol (10 mL) was added Pd/C (0.39 g) at H₂. The reaction mixture was stirred at RT for 16 hours. The resulting mixture was post processed to afford CP24-3 (395.1 mg). MS (ESI, m/z): 226[M+H]⁺.

A solution of CP24-3 (398.0 mg) in acetic acid (10 mL) was stirred at 110° C. for 16 hours. The solution was concentrated to afford CP24-4 (662.7 mg). MS (ESI, m/z): 250[M+H]⁺.

A solution of CP24-4 (217 mg) in borane (1M in THF, 7 mL) was stirred for 4 h at RT, quenched by methanol, post processed to afford CP24-5 (43.3 mg). MS (ESI, m/z): 194[M+H]⁺.

Compound 24 (CP24, confirmed) was synthesized in a manner similar to CP23 with CP24-5. LCMS: m/z: 651[M+H]⁺.

### Example 26

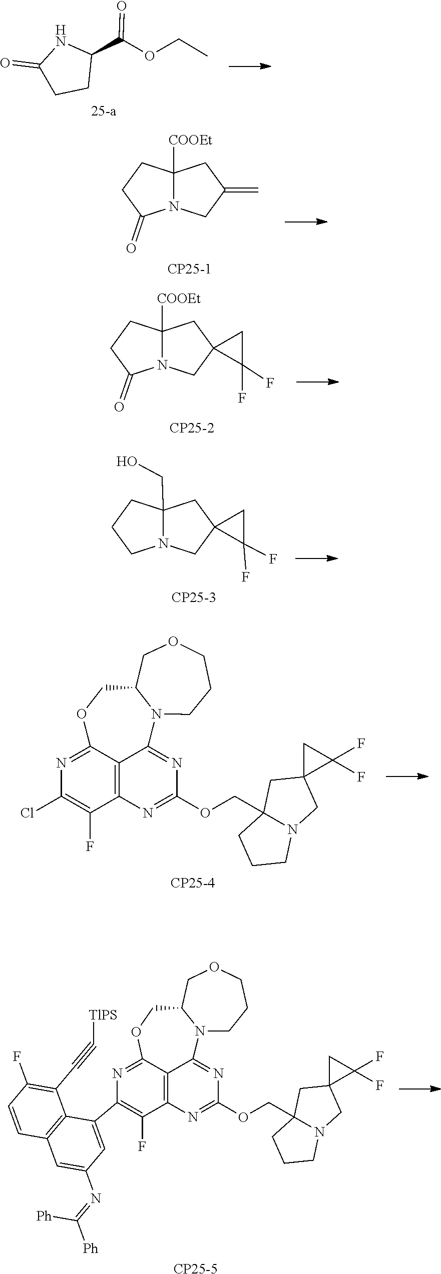

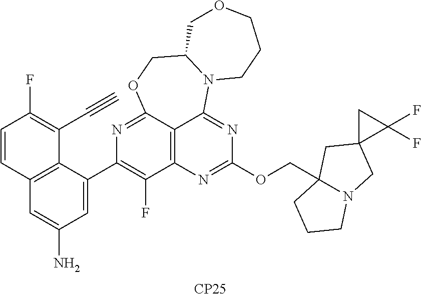

CP25-3 can be purchased or prepared according to the prior art.

Compound 25 (CP25, confirmed) was synthesized in a manner similar to CP23 with CP25-3.

### Example 27

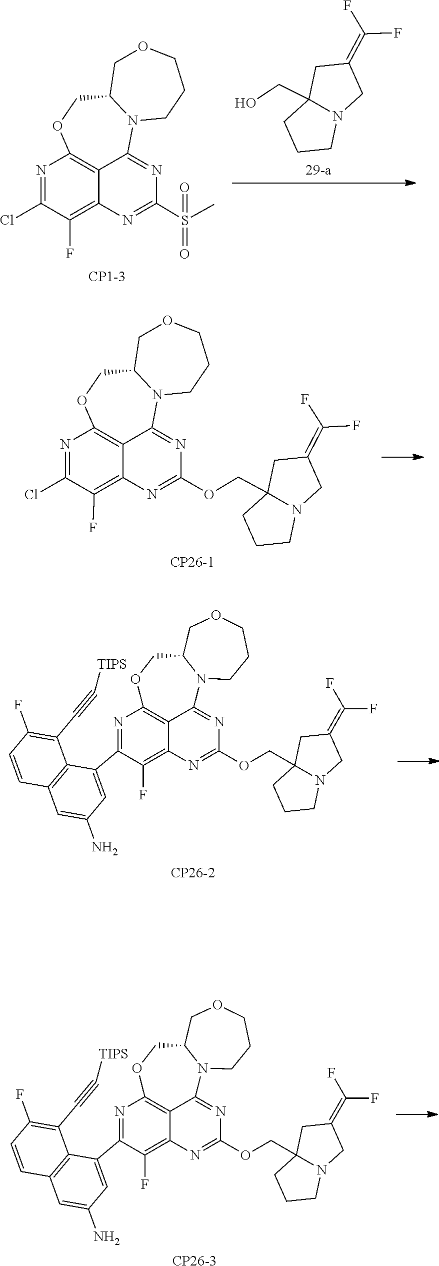

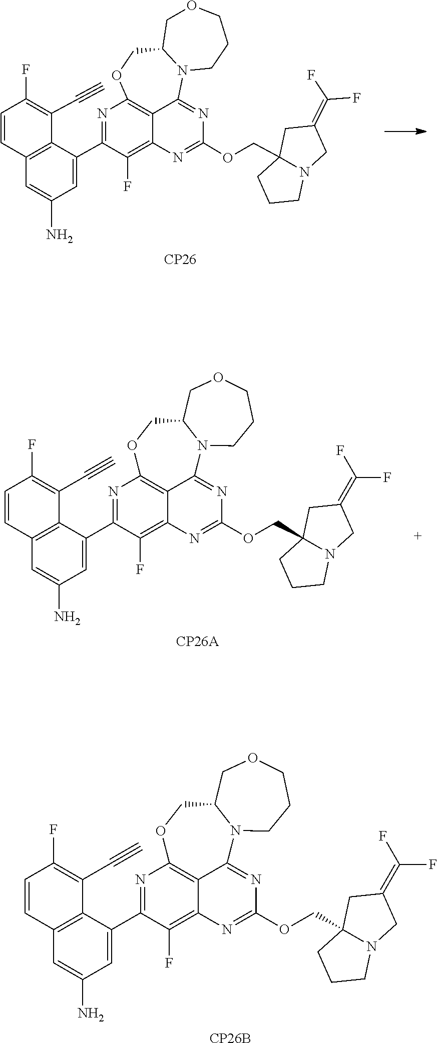

CP26 was synthesized in a manner similar to CP5 with 26-a.

CP26 (0.0171 g) was separated by Prep-HPLC-Gilson with the conditions: Column, CHIRAL ART Cellulose-SA column (2 cm×25 cm, 5 um), mobile phase, (Hex:DCM=3:1)(0.1% DEA)/EtOH (70:30), flowing rate: 18 mL/min. This resulted in Compound 26A (0.0010 g, the first eluting isomer, Retention time: 5.657 min, confirmed) and Compound 26B (0.0069 g, the second eluting isomer, Retention time: 6.437 min, confirmed). LCMS: m/z: 647[M+H]⁺.

### Example 28

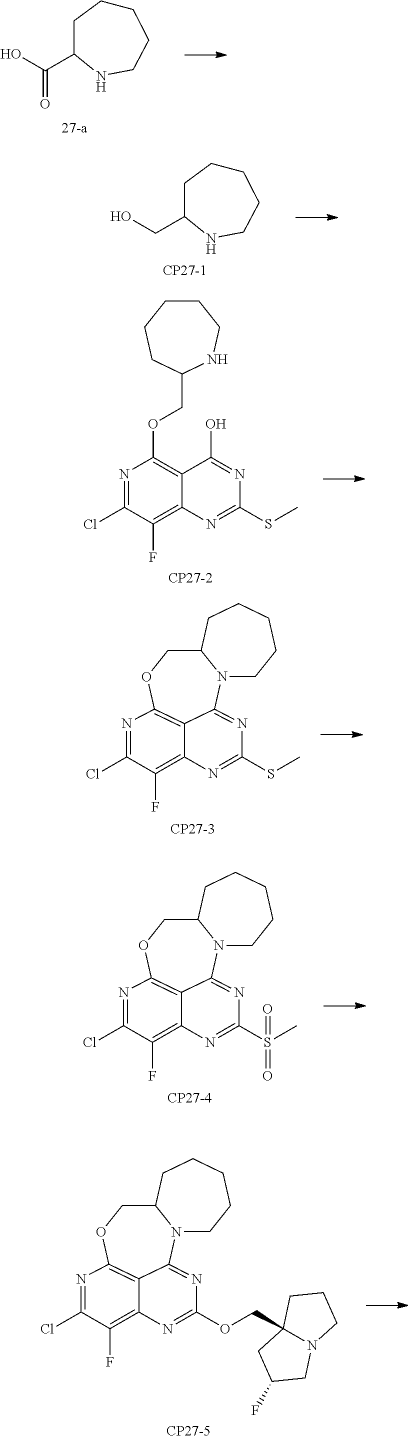

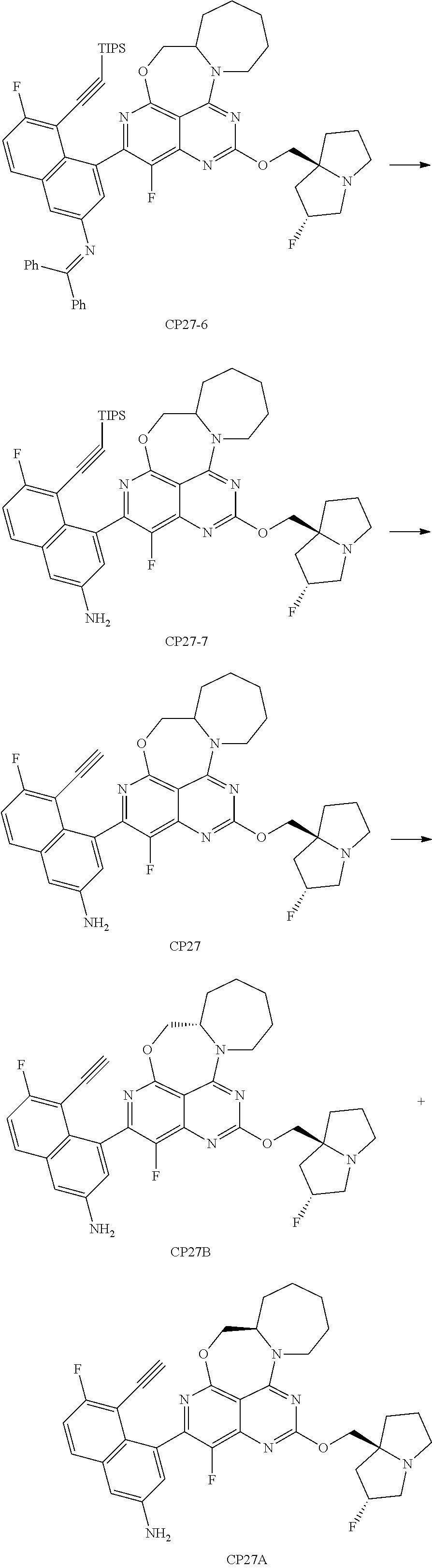

A solution of 27-a (432 mg) in THF (10 mL) was cooled to 0° C., borane (1M in THF, 4 mL) was added dropwise and the resulting mixture was stirred for 16 h at RT. The resulting mixture was post processed to afford CP27-1. LCMS: m/z: 130[M+H]⁺.

Compound 27 (CP27) was synthesized in a manner similar to CP16 with CP27-1. MS (ESI, m/z): 615[M+H]⁺.

CP27 (26 mg) was separated by Prep-HPLC-Gilson with the conditions: Column, CHIRAL ART Cellulose-SA column (2 cm×25 cm, 5 um); mobile phase, (Hex:DCM=3:1)(0.1% DEAY/EtOH (75:25); Flowing rate: 18 mL/min. This resulted in Compound 27A (CP27A, 4.7 mg, the first eluting isomer, Retention time: 5.533 min, confirmed) and Compound 27B (CP271B, 3.9 mg, the second eluting isomer, Retention time: 6.523 min, confirmed).

### Example 29

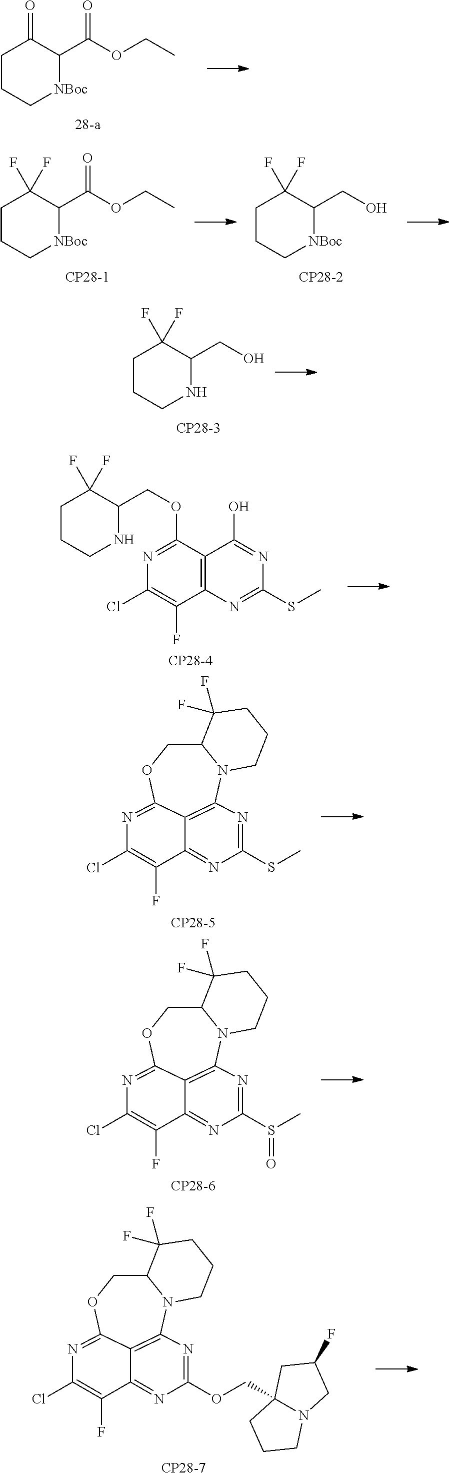

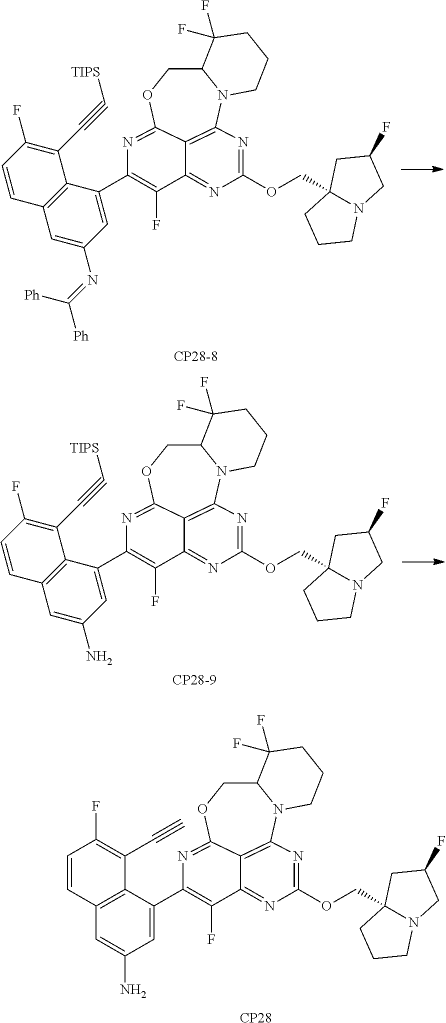

A solution of 28-a (0.95 g) and BAST (1.45 g) in DCM (15 mL) was stirred at RT for 16 hours. The solution was added aq. NaHCO₃ (10 mL). The separated organic layer was post processed to afford CP28-1 (583 mg). MS (ESI, m/z): 294[M+H]⁺.

A solution of CP28-1 (583 mg) in methanol (10 mL) was added NaBH₄ (161 mg) and the resulting mixture was stirred at RT for 16 hours. The mixture was heated to 60° C. and stirred for 11 h. The mixture was post processed to afford CP28-2 (120 mg). MS (ESI, m/z): 252[M+H]⁺.

To a solution of CP28-2 (120 mg) in DCM (5 mL) was added HCl (4M in 1,4-dioxane, 2 mL). The reaction mixture was stirred at RT for 1 hour. The reaction was concentrated to afford CP29-3 (72 mg). MS (ESI, m/z): 152[M+H]⁺.

Compound 28 (CP28, confirmed) was synthesized in a manner similar to CP16 with CP28-3. LCMS: m/z: 637[M+H]⁺.

### Example 30

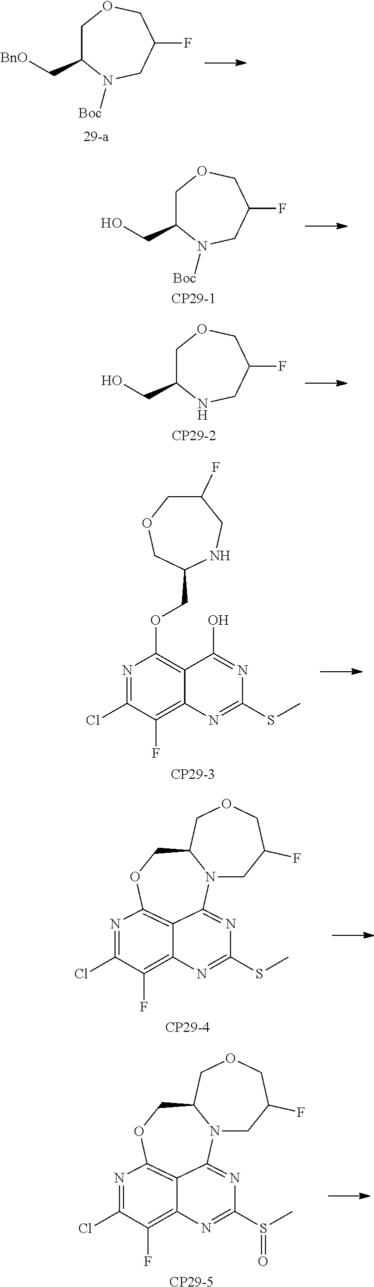

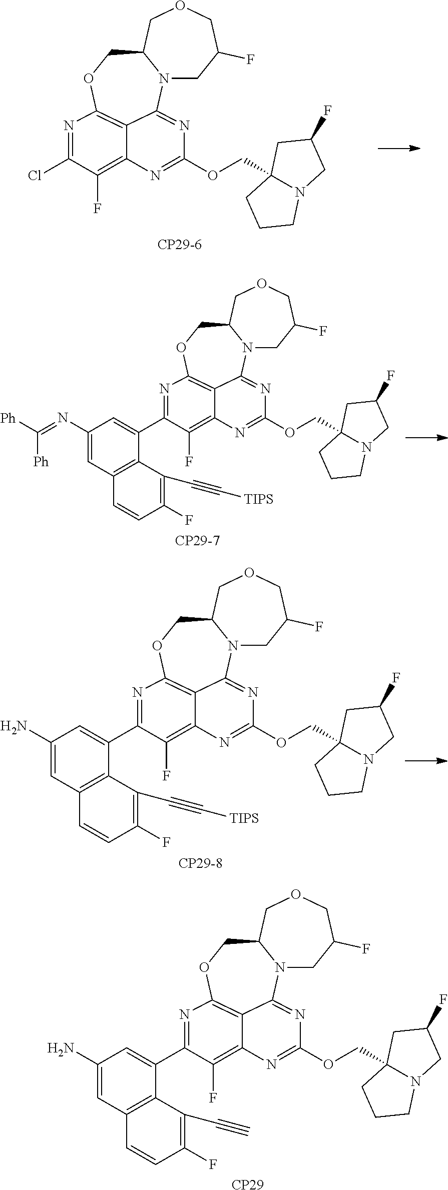

29-a was synthesized in a manner similar to INT16 with N—BOC—O-benzyl-L-seine. PGP-2167C3

To a solution of 29-a (76.30 mg) in methanol (10 mL) was added Pd/C (95 mg) at H₂. The reaction mixture was stirred at RT for 5 hours. The resulting mixture was post processed to afford CP29-1 (77 mg). MS (ESI, m/z): 250[M+H]⁺.

Compound 29 (CP29, confirmed) was synthesized in a manner similar to CP16 with CP29-1. LCMS: m/z: 635[M+H]⁺.

### Example 31

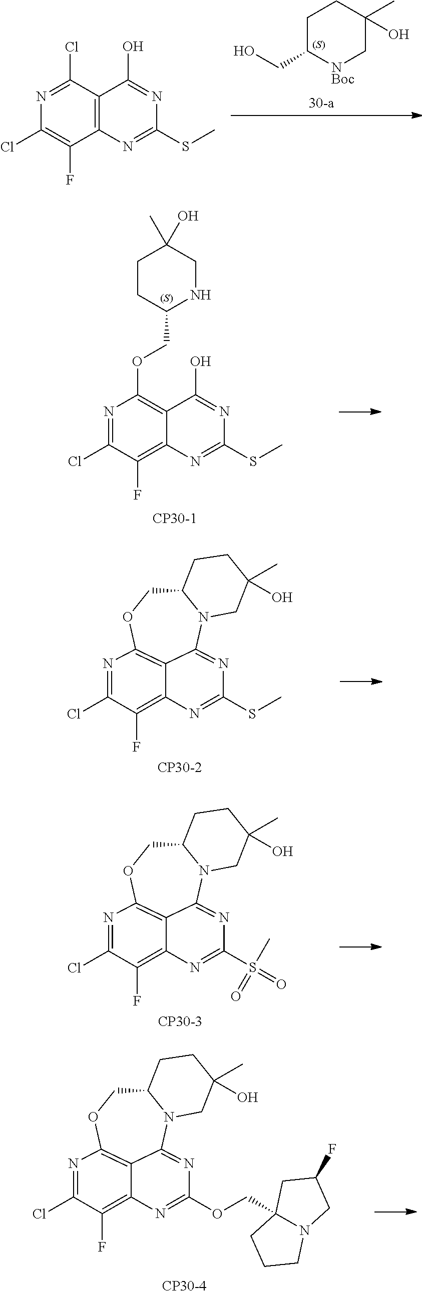

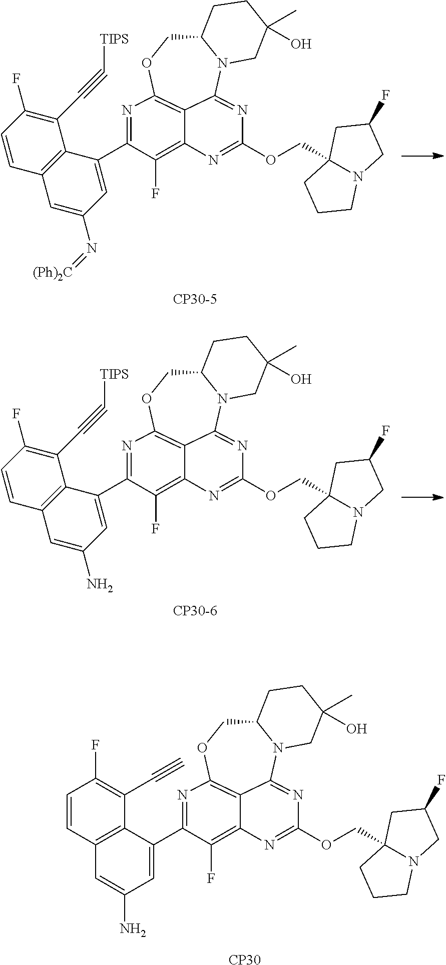

Compound 30(CP30, confirmed) was synthesized in a manner similar to C2 with 30-a.

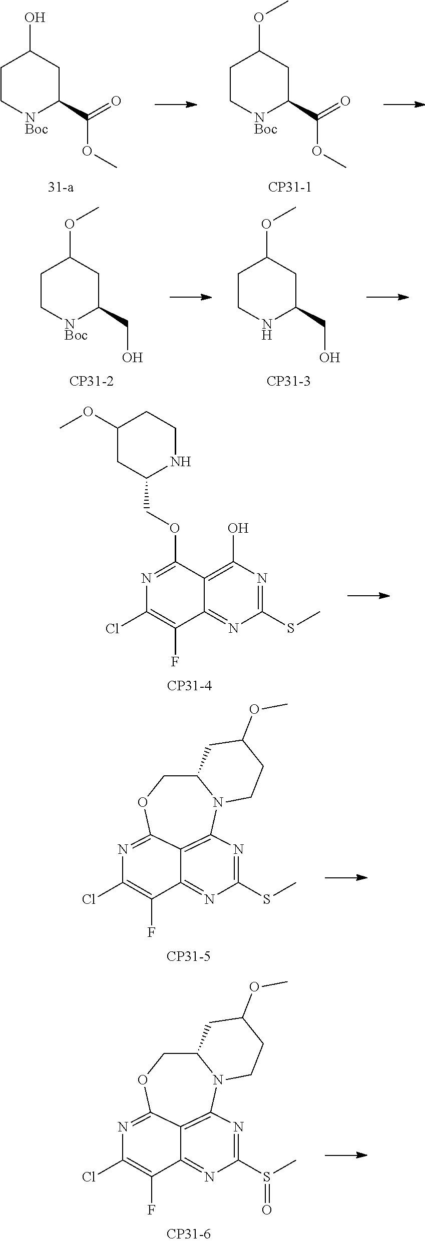

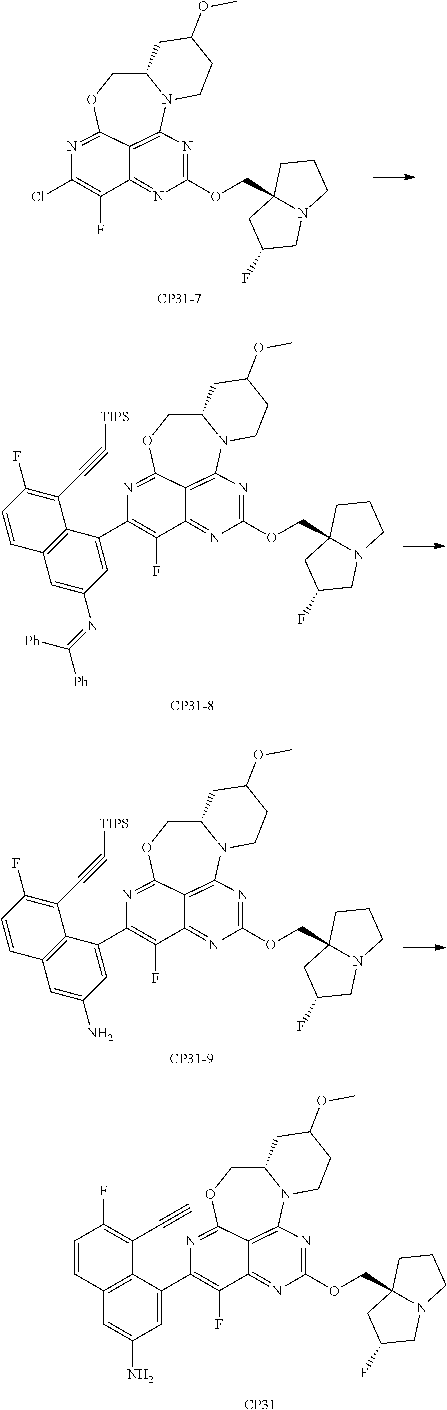

To a solution of 31-a (1191 mg) in DMF (10 mL) were added NaH (205 mg) and methyl iodide (1.16 g). The reaction mixture was stirred at RT for 18 hours, quenched with water (20 mL) and extracted with EA (20 mL). The separated organic layer was post processed to afford CP31-1 (602 mg). MS (ESI, m/z): 274 [M+H]⁺.

To a solution of C31-1 (6021 mg) in THF (10 mL) were added LAH (20 mg) and the resulting mixture was stirred at RT for 30 min. The resulting mixture was added 3 drops of water, 3 drops of 15% aqueous sodium hydroxide solution and 8 drops of water. The solution was filtered. The filtrate was concentrated to afford CP31-2 (424 mg). MS (ESI, m/z): 246[M+H]⁺.

Compound 31 (CP31, confirmed) was synthesized in a manner similar to CP16 with CP31-2. LCMS: m/z: 631[M+H]⁺.

### Example 33

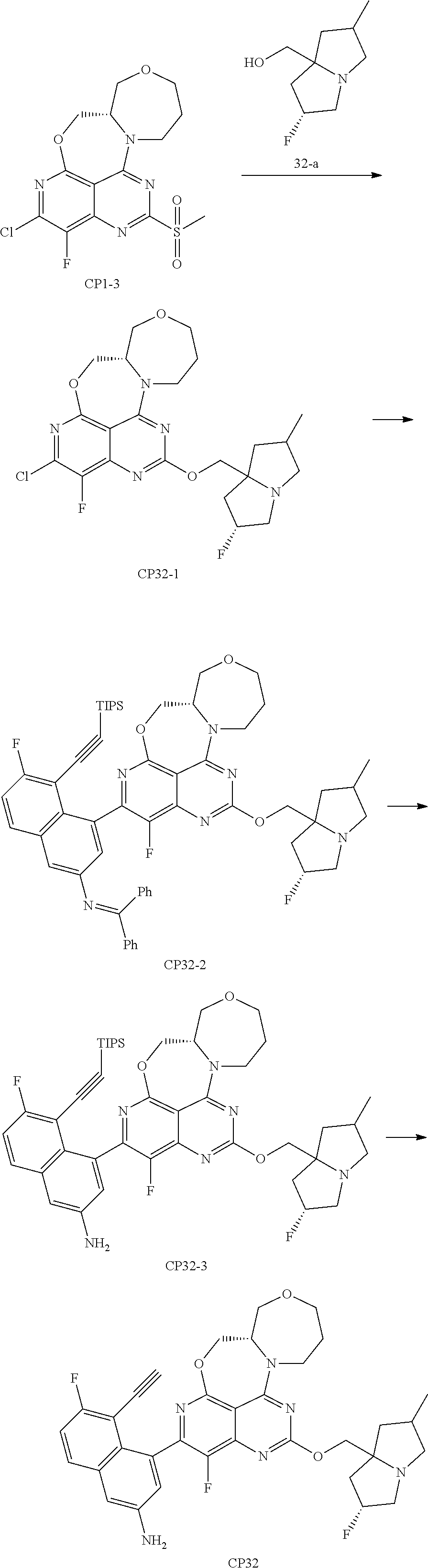

Compound 32 (CP32, confirmed) was synthesized in a manner similar to CP16 with 32-a. LCMS: m/z: 631[M+H]⁺.

### Example 34

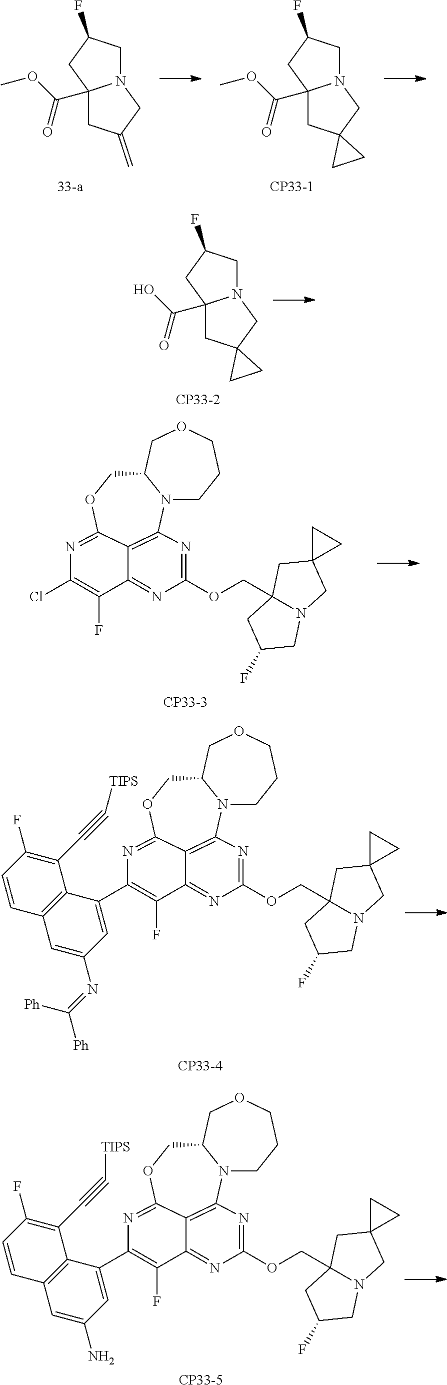

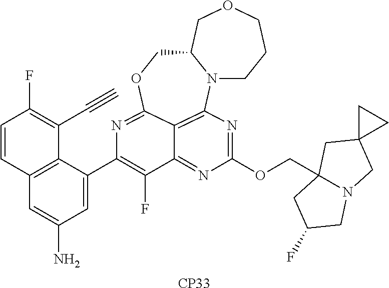

CP33-2 can be purchased or prepared according to the prior art.

Compound 33 (CP33, confirmed) was synthesized in a manner similar to CP27 with CP33-2. LCMS: m/z: 643[M+H]⁺.

### Example 35

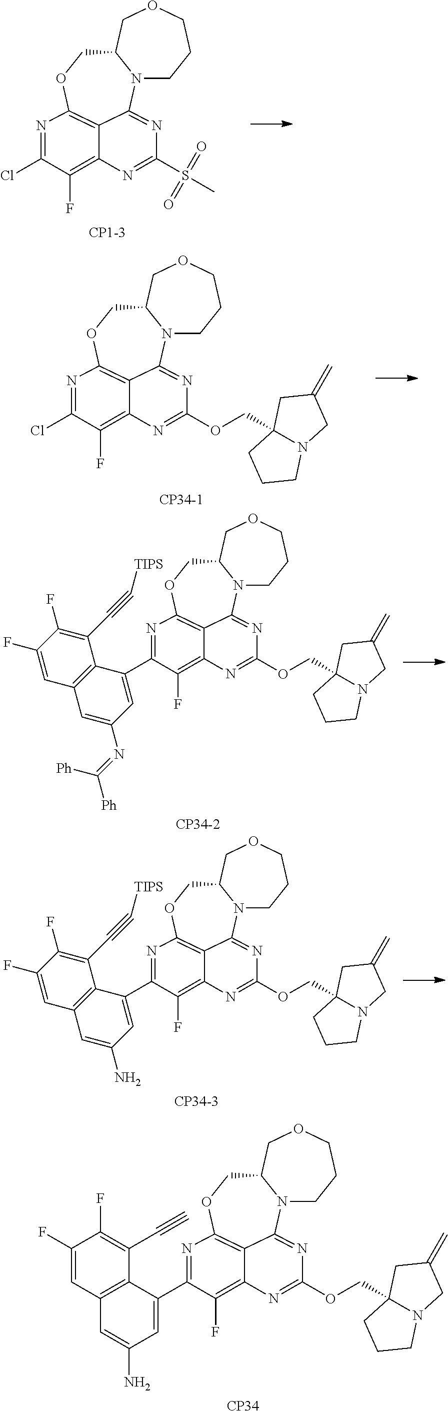

Compound 34 (CP34, confirmed) was synthesized in a manner similar to CP5. LCMS: m/z: 629[M+H]⁺.

### Example 36

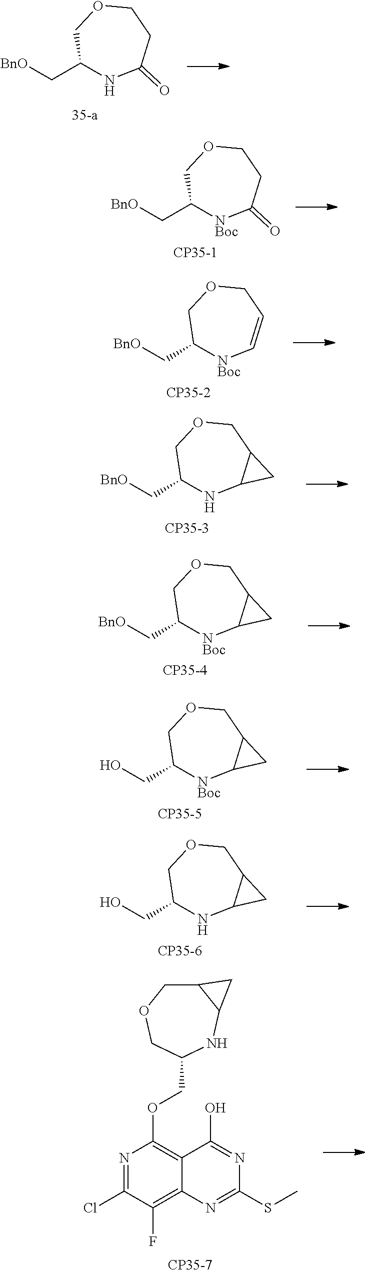

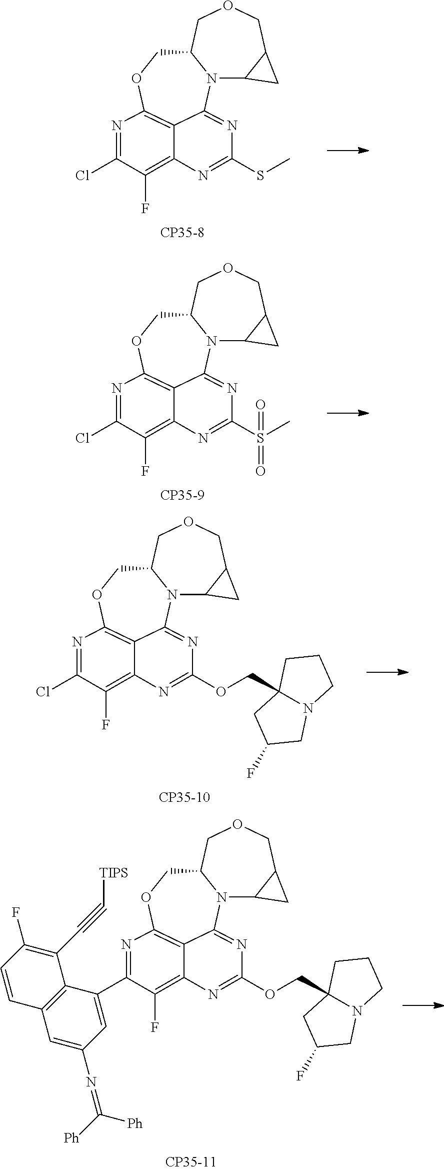

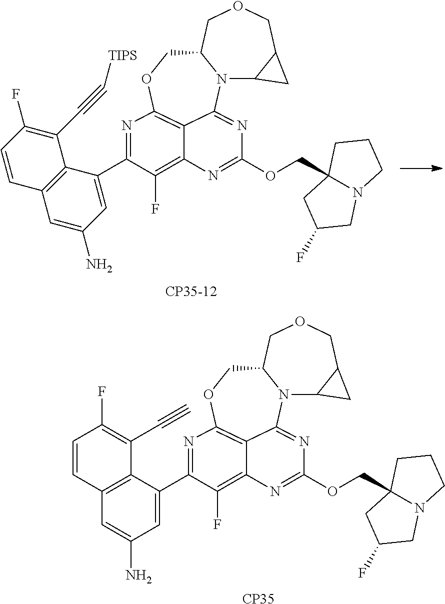

A solution of 35-a (5.15 g), DIEA (8.75 g) and DMAP (0.35 g) and Boc₂O (5.67 g) in DCM (100 mL) was stirred for 40 h at 40° C. Upon completion, the mixture was post processed to afford CP35-1 (3.29 g). MS (ESI, m/z): 336[M+H]⁺.

A solution of CP35-1 (3 g) in toluene (50 mL) was cooled to −78° C. Lithium triethylborohydride (1.4214 g) was added and the resulting mixture was stirred at −78° C. for 4.5h, DMAP (60 mg), DIEA (6.02 g) and trifluoroacetic anhydride (7.47 g) were added and stirred overnight at RT. The solution was diluted with NaHCO₃ (100 mL). The separated organic layer was post processed to afford CP35-2 (1.45 g). MS (ESI, m/z): 320[M+H]⁺.

A solution of CP35-2 (1.45 g) in DCM (30 mL) was cooled to 10° C. Diethylzinc (1M in n-hexane, 13 mL) was added dropwise and stirred for 30 min at RT, the reaction mixture was cooled to 0° C. and diiodomethane (5.19 g) was added dropwise, warmed to RT slowly and stirred for 16 h. The system was cooled down. 200 mL of saturated ammonium chloride solution was added for extraction. The separated organic layer was post processed to afford CP35-3 (1.27 g). MS (ESI, m/z): 234[M+H]⁺.

A solution of CP35-3 (1.27 g) and DMAP (0.71 g), Boc₂O (1.67 g) in DCM (20 mL) was stirred for 16 h at RT. The reaction mixture was heated to 40° C. and stirred for 24 h. The separated organic phase was post processed to afford CP35-4 (578 mg). MS (ESI, m/z): 334[M+H]⁺.

Compound 35 (CP35, confirmed) was synthesized in a manner similar to CP29 with CP35-4. MS: m/z: 629[M+H]⁺.

### Example 37

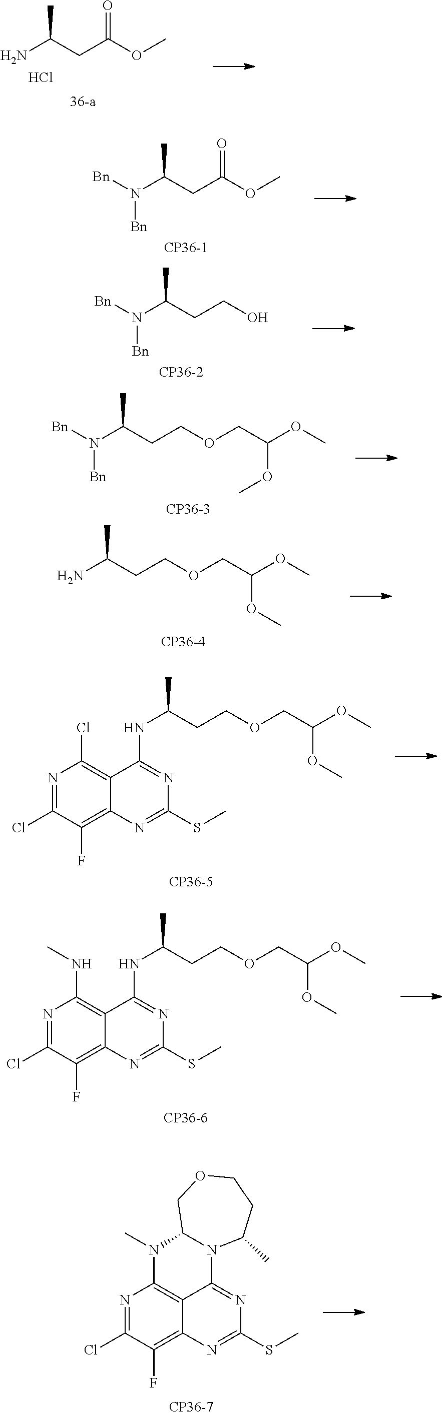

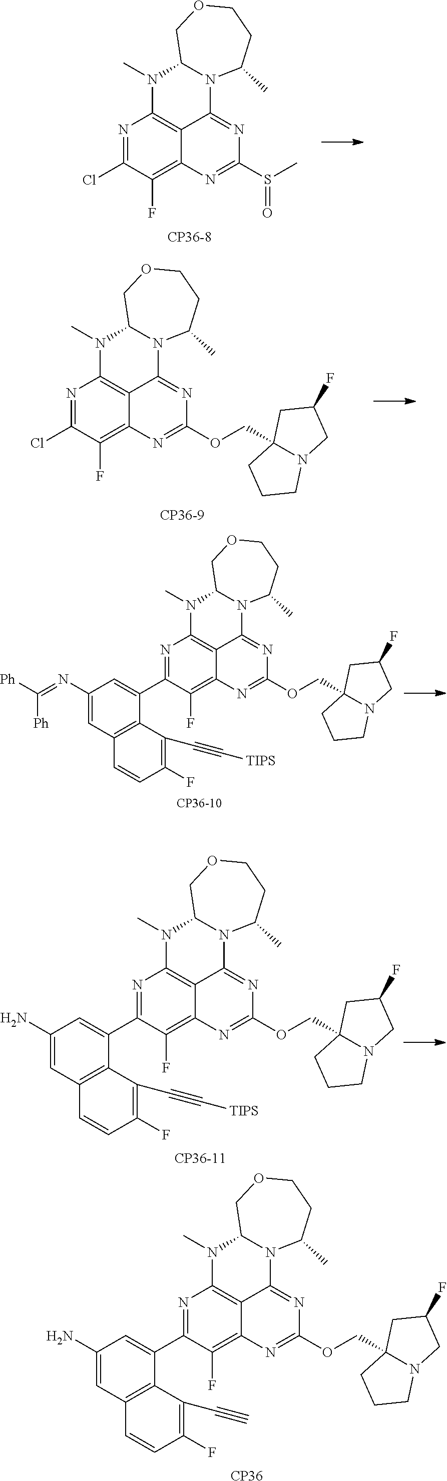

A solution of 36-a (2.90 g), K₂CO₃ (13.51 g) and phenylmethyl bromide (8.91 g) in CH₃CN (30 mL) was stirred for 24 h at 80° C. Upon completion, the mixture was post processed to afford CP36-1 (7.49 g). MS (ESI, m/z): 298[M+H]⁺.

To a solution of CP36-1 (7.49 g) in THF (20 mL) was added LAH (0.90 g) in batches at 0° C. and the resulting mixture was stirred for 1 hours, quenched by water, 15% NaOH (0.8 mL) and water (2.4 mL). The solution was post processed to afford CP36-2 (4.56 g). MS (ESI, m/z): 270[M+H]⁺.

To a solution of CP36-2 (4.56 g) in THF (30 mL) was added NaH (2396 mg) in batches at 0° C. and the resulting mixture was stirred at RT for 20 min. 2-bromo-1,1-dimethoxyethane (5.80 g), DMAP (1.08 g) and TBAI (0.66 g) were added and the resulting mixture was stirred at 70° C. for 17 h, quenched by water and extracted with EA. The separated organic layer was post processed to afford CP36-3 (5.06 g). MS (ESI, m/z): 358[M+H]⁺.

To a solution of CP36-3 (5.06 g) in ethanol (30 mL) was added Pd(OH)₂/C (0.54 g) at H₂. The reaction mixture was stirred at RT for 92 hours. The resulting mixture was post processed to afford CP36-4 (2.34 g). MS (ESI, m/z): 178[M+H]⁺.

CP36 was synthesized in a manner similar to CP46 with CP36-4 and purified by Prep-HPLC (Agela Durashell C18, phase A: 0.05% NH₄OH in water, phase B: CH₃CN, gradient: 25% B to 58% B in 35 min at a flow rate of 40 mL/min, 225 nm) to freeze-dried to afford Compound 36 (CP36, 0.0240 g, confirmed). MS (ESI, m/z): 630[M+H]⁺.

### Example 38

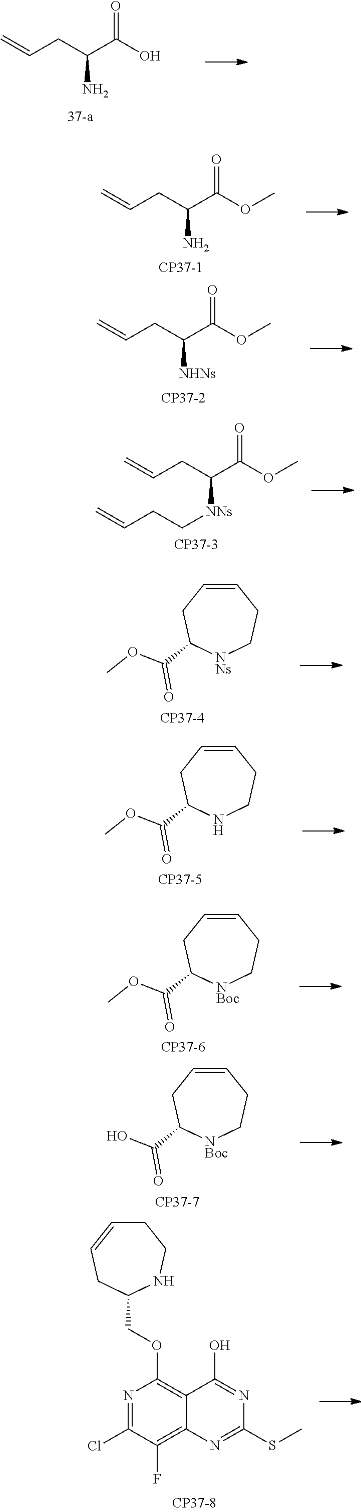

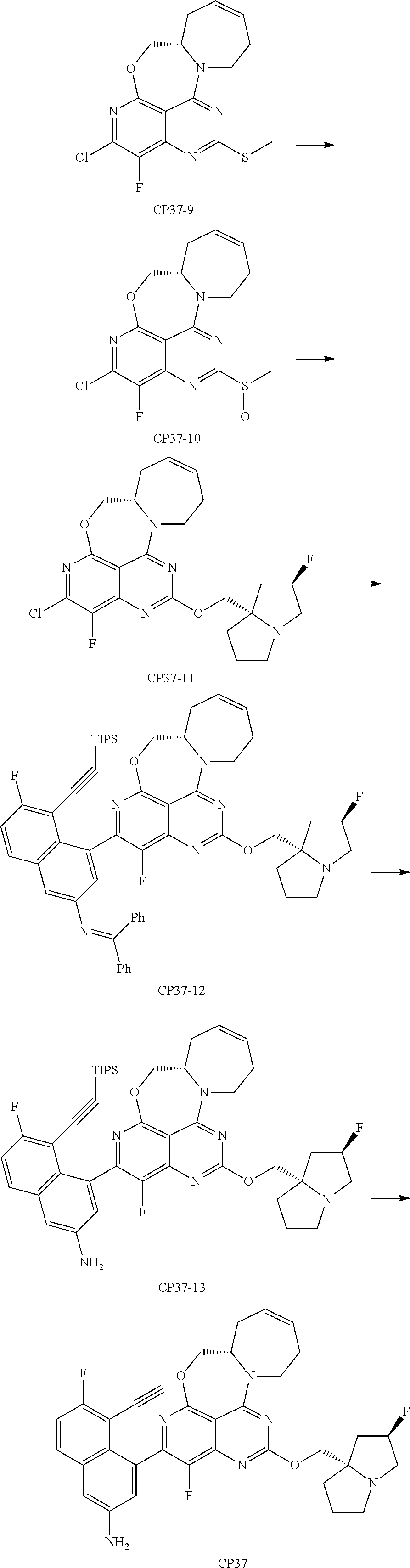

To a solution of 37-a (3.00 g) in methanol (50 mL) was added thionyl chloride (75.81 mmol) dropwise at 0° C. and the resulting mixture was stirred at RT for 16 h. The resulting mixture was concentrated to afford CP37-1 (4.34 g, HC salt). MS (ESI, m/z): 130[M+H]⁺.

To a solution of CP37-1 (4.34 g) in DCM (65 mL) was added TEA (68.34 mmol). The resulting white suspension was cooled to 0° C. and 4-nitrobenzenesulfonyl chloride (6.09 g) was added in portions. The mixture was stirred at RT for 16 h, quenched with water and extracted with DCM. The separated organic layer was post processed to afford CP37-2 (8.02 g). MS (ESI, m/z): 313[M−1]⁻.

To a solution of CP37-2 (3.01 g) in tetrahydrofuran (50 mL) at 0° C. were added but-3-en-1-ol (0.98 g) and triphenylphosphine (5.07 g). Subsequently, DIAD (3.84 g) was added slowly over 10 min. The reaction mixture was allowed to warm to RT and stirred for 16 h, quenched with water and extracted with EA (100 mL). The separated organic layer was post processed to afford CP37-3 (4.19 g).

A solution of CP37-3 (4.19 g) and Grubbs Catalyst 2nd Generation (1.03 g) in DCM (200 mL) was stirred at 50° C. for 16 h. Upon completion, the resulting mixture was post processed to afford CP37-4 (3.427 g).

To a solution of CP37-4 (3.427 g) in methanol (150 mL) were added Cs₂CO₃ (23.05 g) and mercaptoacetic acid (3.84 g). The mixture was stirred at RT for 16 h. The resulting mixture was post processed to afford CP37-5 which was used to next step directly. LCMS: m/z: 156[M+H]⁺.

To a solution of CP37-5 in THF (30 mL) and water (30 mL) was added di-tert-butyl dicarbonate (2.94 g) and the solution was stirred at RT for 1 h. The resulting mixture was extracted with EA (100 mL). The separated organic layer was post processed to afford CP37-6 (943 mg). MS (ESI, m/z): 256[M+H]⁺.

To a solution of CP37-6 (943 mg) in THF (20 mL) was added LiAlH₄ (200 mg) and the resulting mixture was stirred at RT for 2 h, quenched by water (0.2 mL), aq. 15% NaOH (0.2 mL) and water (0.6 mL) subsequently. The solution was post processed to afford CP37-7 (498 mg). MS (ESI, m/z): 228[M+H]⁺.

Compound 37 (CP37, confirmed) was synthesized in a manner similar to CP16 with CP37-7. MS (ESI, m/z): 613[M+H]⁺.

### Example 39

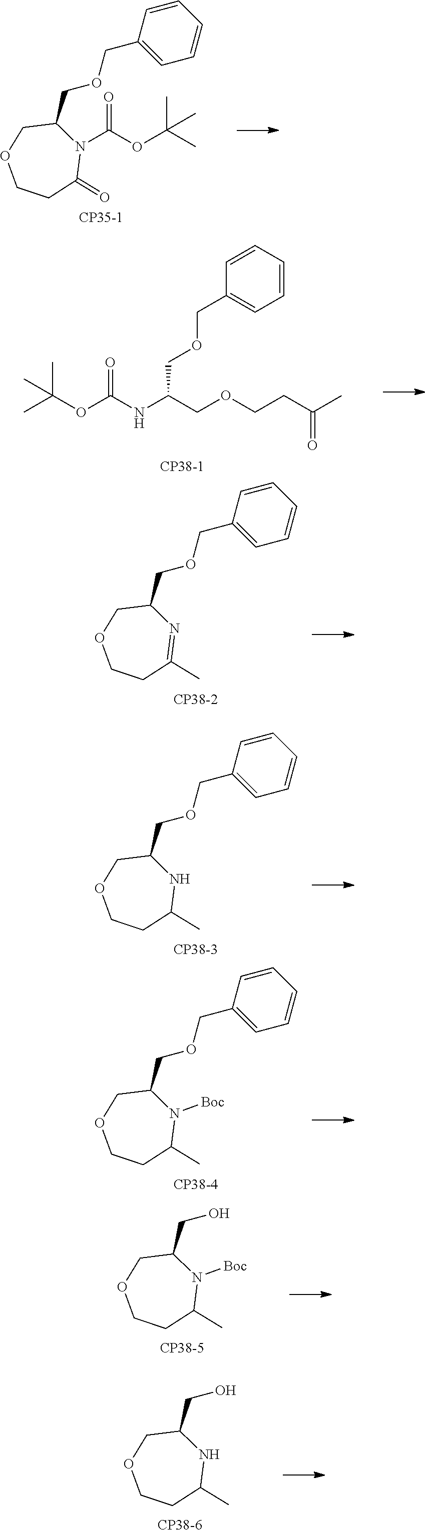

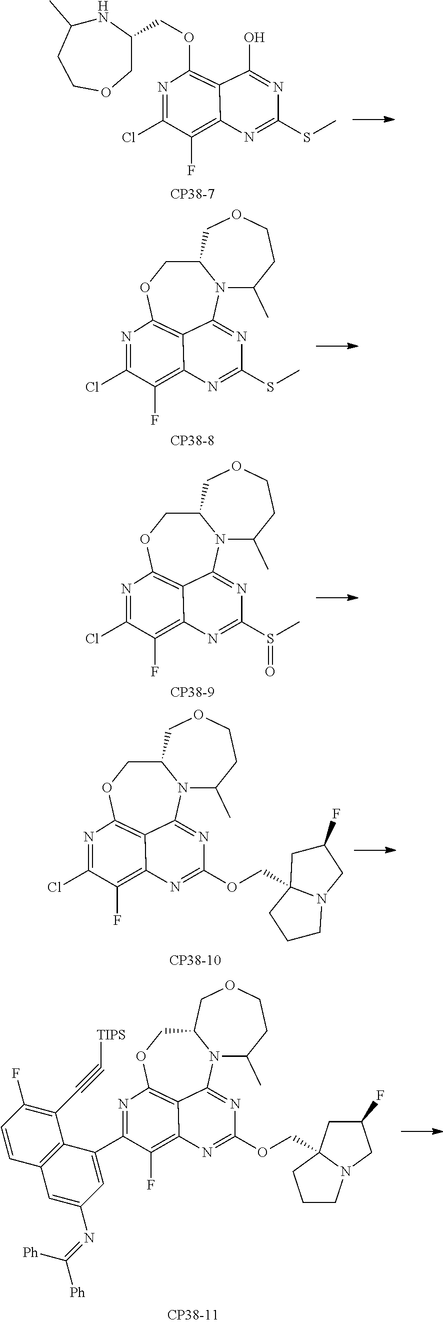

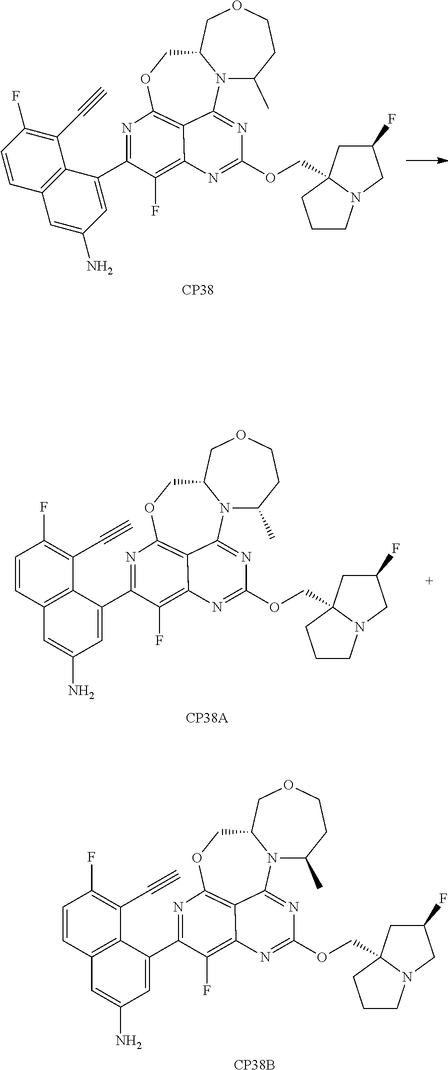

To a solution of CP35-1 (847 mg) in THF (8 mL) was added methylmagnesium bromide (3 mmol. 3M in THF) at −78° C. The reaction mixture was stirred for 3.5 hour and quenched with aq. NH₄Cl (20 mL). The organic layer was post processed to give CP38-1 (680 mg). MS (ESI, m/z): 352[M+H]⁺.

To a solution of CP38-1 (680 mg) in DCM (8 mL) was added TFA (2 mL). The reaction mixture was stirred for 3.5 hour and concentrated to give CP38-2 (crude) which was used to next step. MS (ESI, m/z): 234[M+H]⁺.

To a solution of CP38-2 (crude) in ethylene chloride (5 mL) was added sodium triacetoxyborohydride (1.26 g). The reaction mixture was stirred for 5 hour, quenched with MeOH (10 mL) and water (2 mL), and concentrated to give CP38-3 (crude) which was used to next step. MS (ESI, m/z): 236[M+H]⁺.

CP38-8 was synthesized in a manner similar to CP35 with CP38-3.

CP38-11 was synthesized in a manner similar to CP16-7 with CP38-8.

Compound 38 (CP38) was synthesized in a manner similar to CP35 with CP38-11, and separated by Prep-HPLC (Agela Durashell C18, 30 mm×250 mm, 10 um, phase A: 0.05% NH₄OH, phase B: CH₃CN, gradient: 20% B to 50% B in 34 min at a flow rate of 40 ml/min, 230 nm) to freeze-dried to afford Compound 38A (CP38A, 16.6 mg, Retention time: 35.9 min, confirmed) and Compound 38B (CP38B, 3.1 mg, Retention time: 37.5 min, confirmed). LCMS: 631[M+H]⁺.

### Example 40

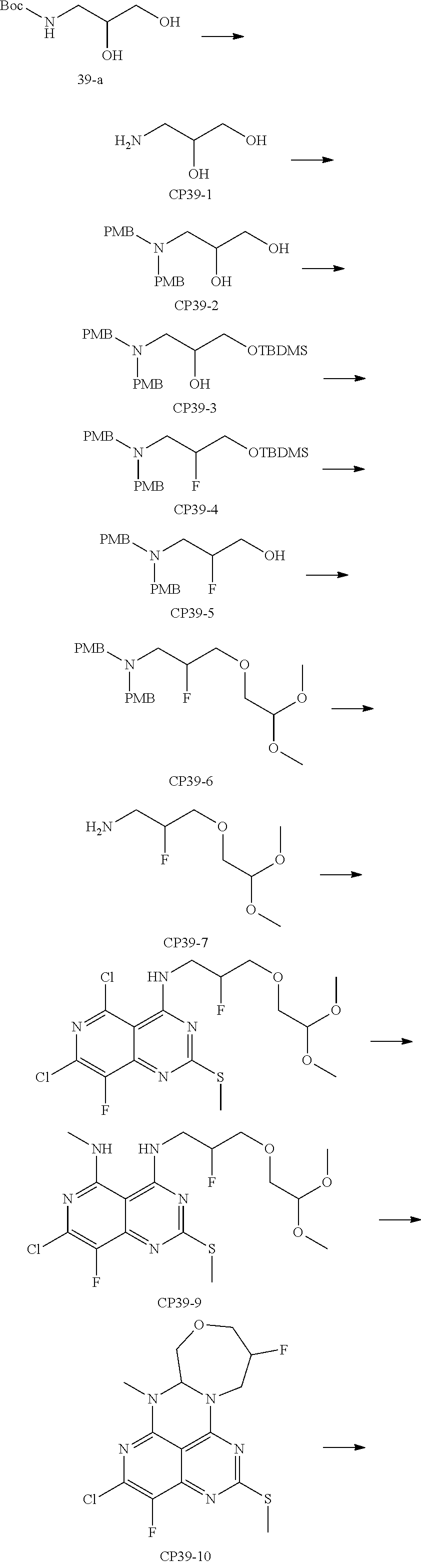

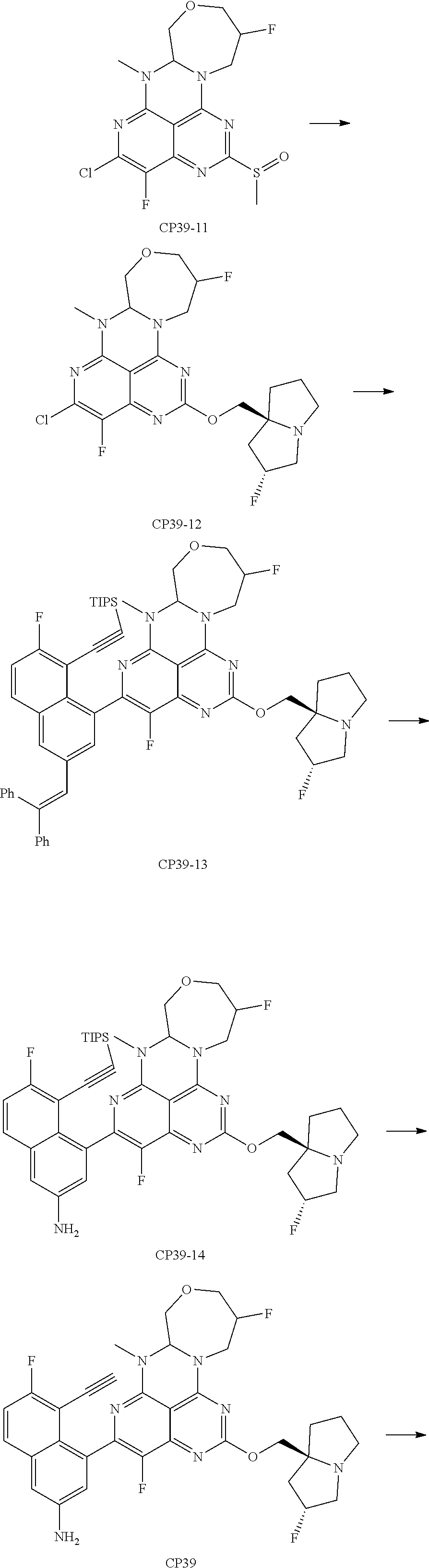

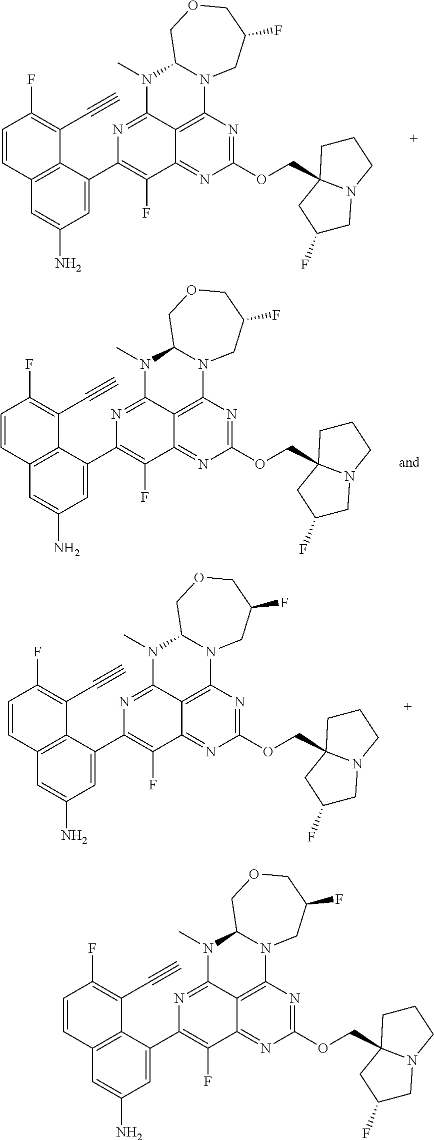

A solution of 39-a (53.03 g) in DCM (300 mL) was stirred at 0° C. The mixture was added HCl (300 ml, 4M in 1,4-dioxane) at 0° C. and stirred at RT for 2 h. The mixture was concentrated to afford the crude CP39-1 (35.12 g,). MS: m/z: 92[M+H]⁺.

A solution of CP39-1 (29.32 g), TEA (104.89 g) and p-anisaldehyde (112.00 g) in MeOH (300 mL) was stirred at 0° C. The mixture was added sodium triacetoxyborohydride (137.83 g) at 0° C. and stirred at 0° C. for 1 h. The mixture was stirred at 40° C. for 24 h, quenched with water, extracted with EA and post processed to afford CP39-2 (25.50 g). MS: m/z: 332[M+H]⁺.

A solution of CP39-2 (5.50 g), TEA (2.28 g), DMAP (0.29 g) and TBDMSCl (2.54 g) in DCM (60 mL) was stirred at RT for 16 h, quenched with water, extracted with DCM and post processed to afford CP39-3 (5.78 g). MS: m/z: 446[M+H]⁺.

A solution of CP39-3 (3.46 g) in DCM (40 mL) was added DAST (1.57 g) at −10° C. The reaction mixture was stirred at −10° C. for 1 h, quenched with 10% Na₂CO₃ solution, extracted with DCM and post processed to afford CP39-4 (1.57 g). MS: m/z: 448[M+H]⁺.

To a solution of CP39-4 (1.57 g) in DMF (25 mL) was added CsF (6861 mg). The reaction mixture was stirred for 5 hours at 30° C. The solution was quenched with water and extracted with EA and post processed to afford CP39-5 (1048 mg). MS: m/z: 334[M+H]⁺.

A solution of CP39-5 (300 mg) in THF (10 mL) was stirred at 0° C. The mixture was added sodium hydride (139 mg, 60%) at 0° C. and stirred at RT for 0.5h. The mixture was added DMAP (32 mg), TBAI (44 mg) and bromoacetaldehyde dimethyl acetal (465 mg) and stirred at 70° C. for 16 h. The mixture was allowed to cool to RT, quenched with water, extracted with EA and post processed to afford CP39-6 (247 mg). MS: m/z: 422[M+H]⁺.

Compound 39 (CP39) was synthesized in a manner similar to CP46 with CP39-6 and separated by Prep-TLC (DCM/MeOH=10:1) to give the first eluted Compound 39A (CP39A, 143 mg) and the second eluted Compound 39B (17 mg). LCMS: m/z: 634[M+H]⁺.

### Example 41

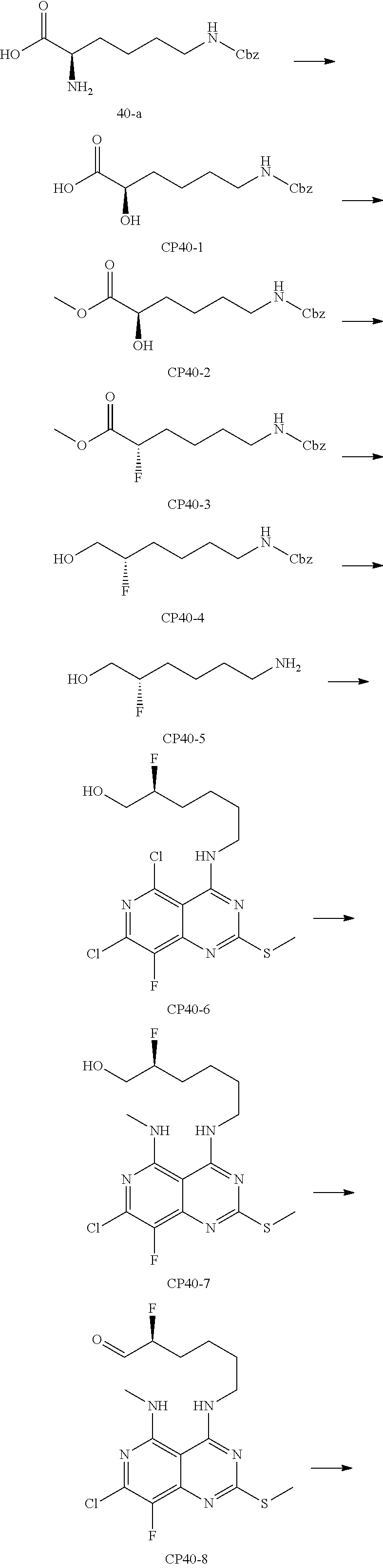

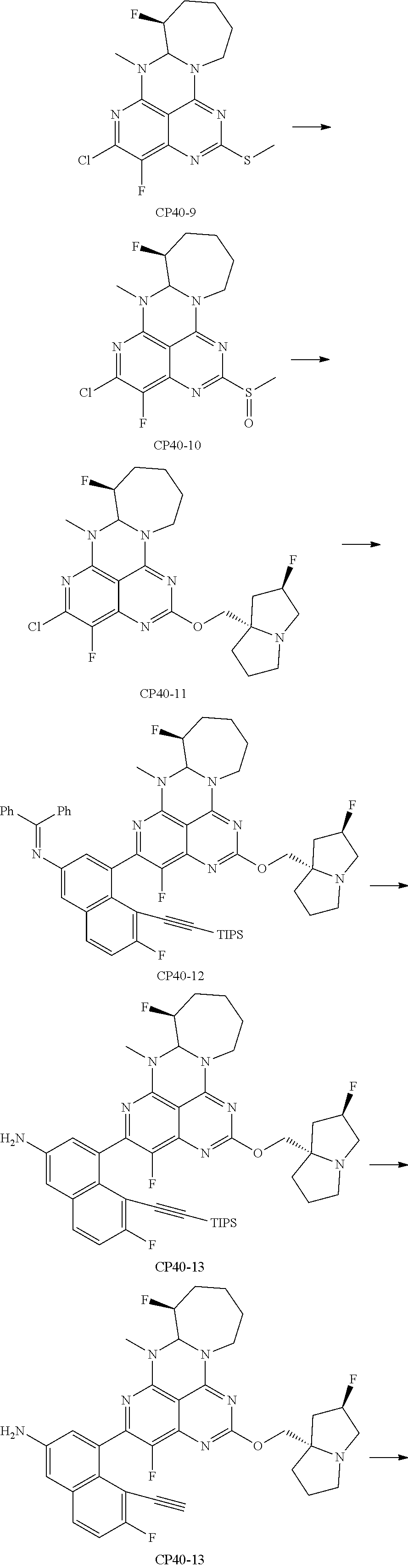

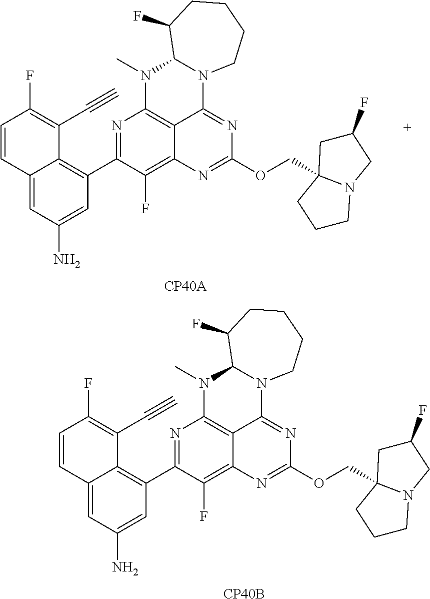

A solution of 40-a (20.42 g), acetic acid (600 mL) and water (600 mL) was cooled to 0° C. to 5° C. Sodium nitrite (25.14 g) in water (50 mL) was added slowly to the above solution. The reaction was stirred for 7 h. The resulting solution was diluted with 500 mL of EA and washed with water (200 mL). The organic phase was dried, filtered and concentrated. The residue was dissolved in MeOH (600 mL) and water (600 mL) at RT. K₂CO₃ (15.43 g) was added. The mixture was stirred for 24 h. The resulting solution was adjusted pH=1 to 2 with 1N HCl aqueous solution. The mixture was extracted with EA. The combined organic layer was concentrated to afford CP40-1 (23.37 g, crude). The crude product was used in the next step. LCMS (ESI, m/z): 282[M+H].

To a solution of CP40-1 (5.11 g) in methanol (100 mL) was added thionyl chloride (2.1611 g) at 0° C. The mixture was stirred for 16 h at reflux. The resulting solution was concentrated. The residue was purified by silica gel column chromatography eluting with MeOH/DCM (0~5%, v/v) to afford CP40-2 (1.98 g). LCMS (ESI, m/z): 296[M+H]⁺.

CP40-2 (1.98 g) was dissolved in DCM (30 mL) under nitrogen at 0° C. A solution of DAST (3.2420 g) in DCM (30 mL) was added slowly to the above solution. The resulting mixture was warmed to RT. The reaction was stirred for 20 h and quenched with saturated NaHCO₃ solution (100 mL) at 0 to 5° C. The resulting solution was diluted with 100 mL of DCM. The organic phase was washed with brine, dried, filtered and concentrated. The residue was purified by C18 gel chromatography eluting with H₂O/CH₃CN to afford CP40-3 (747 mg). LCMS (ESI, m/z): 298[M+H]⁺.

To a solution of CP40-3 (643 mg) in THF (3 mL) was added LAH (247 mg) at 0° C. The mixture was stirred for 1 h at 0° C. and quenched with Na₂SO₄ hydrated at 0-5° C. The resulting mixture was filtered and the filtrate was concentrated. The residue was purified by silica gel column chromatography eluting with EA/HEX (0 to 50%, v/v) to afford CP40-4 (423 mg). LCMS (ESI, m/z): 270[M+H]⁺.

A solution of CP40-4 (1.92 g) in methanol (40 mL) was added palladium hydroxide (354 mg), purged with N₂ and pressurized with H₂. The mixture was stirred for 20 h at RT. After completion, the resulting mixture was filtered and the filter cake was washed with methanol. The filtrate was collected and removed to afford CP40-5 (1.31 g). The crude product was used in the next step. LCMS (ESI, m/z): 136[M+H]⁺.

A solution of INT6 (894 mg), DIEA (828 mg) and POCl₃ (432 mg) in toluene (20 mL) was stirred at 100° C. for 2 h. The resulting mixture was concentrated. The residue was added to a mixture of CP40-5 (0.285 g) and DIEA (874 mg) in DCM (30 mL) at −10 to 20° C. The resulting mixture was warmed to RT. The reaction was stirred for 0.5 h. The resulting solution was diluted with 50 mL of DCM. The organic phase was washed with brine, dried over anhydrous Na₂SO₄, filtered and concentrated. The residue was purified by prep-TLC with (EA:HEX=1:2, v/v) to afford CP40-6 (253 mg). LCMS (ESI, m/z): 397[M+H]⁺.

To a solution of CP40-6 (2.09 g) in DMAc (40 mL) was added methylamine hydrochloride (532.8072 mg) and Cs₂CO₃ (3.4282 g). The mixture was stirred at 80° C. for 3 h. After cooling to RT, the resulting mixture was diluted with water (400 mL) and filtered. The filter cake was dried to afford CP40-7 (1.55 g). LCMS (ESI, m/z): 392[M+H]⁺.

To a solution of CP40-7 (1.55 g) and TEA (2.06 g) in DMSO (60 mL) and DCM (30 mL) was added pyridine sulfur trioxide (2.00 g) at −10 to 20° C. The resulting mixture was warmed to RT. The reaction was stirred for 3 h. The resulting solution was diluted with 30 mL of DCM. The organic phase was washed with brine, dried, filtered and concentrated. The residue was purified by silica gel column chromatography eluting with DCM/EA/HEX (1/1/1, v/v) to afford CP40-8 (1.10 g). LCMS (ESI, m/z): 390[M+H]⁺.

A solution of CP40-8 (1.10 g) and p-TsOH·H₂O (661 mg) in DMSO (30 mL) was stirred at 80° C. for 20 h. The solution was diluted with EA and washed with brine. The organic layer was dried, filtered and concentrated. The residue was purified by a silica gel column chromatography (eluent: 0~30% EA in Hexane) to afford CP40-9 (262 mg). LCMS (ESI, m/z): 372[M+H]⁺.

CP40 was synthesized in a manner similar to CP46 with CP40-9, purified and separated by Prep-HPLC (YMC-Triart C18-S12 nm, 50 mm×250 mm, 7 um, phase A: 0.05% ammonium hydroxide in water, phase B: CH₃CN, Gradient: 40% B to 73% B in 34 min at a flow rate of 70 mL/min, 224 nm) and freeze-dried to afford Compound 40A (CP40A, the first peak, 3.4 mg, confirmed) and Compound 40B (CP40B, the second peak, 49.2 mg, confirmed). MS (ESI, m/z): 632[M+H]⁺.

¹HNMR (400 MHz, DMSO-d₆) δ7.75 (dd, J=8.9, 6.0 Hz, 1H), 7.32 (m, 1H), 7.09-6.95 (m, 2H), 5.60 (s, 2H), 5.34 (dd, J=12.6, 7.1 Hz, 1H), 4.88 (d, J=47.7 Hz, 1H), 4.43 (s, 1H), 4.20-3.89 (m, 3H), 3.12-3.07 (m, 3H), 3.03 (d, J=4.2 Hz, 1H), 2.83 (d, J=6.4 Hz, 1H), 2.16-1.64 (m, 14H), 1.62-1.32 (m, 2H).

### Example 42

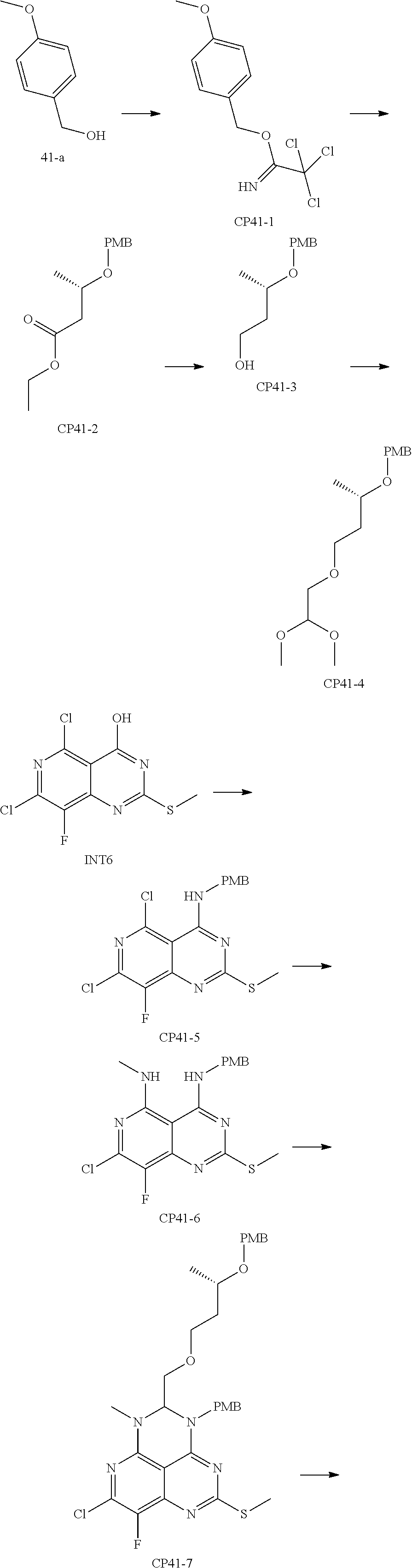

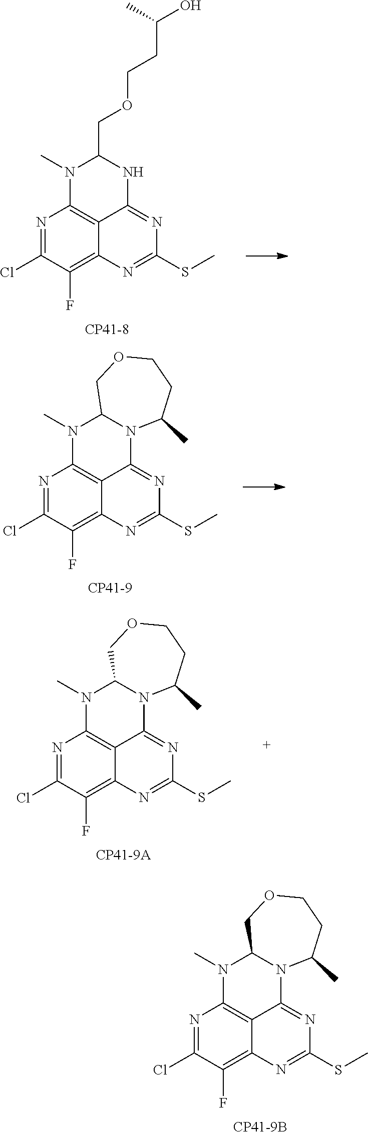 [SMILES: CSc1nc2c3c(nc(Cl)c(F)c3n1)N(C)C(COCC[C@H](C)O)N2 ; CSc1nc2c3c(nc(Cl)c(F)c3n1)N(C)C1COCC[C@@H](C)N21 ; CSc1nc2c3c(nc(Cl)c(F)c3n1)N(C)[C@@H]1COCC[C@@H](C)N21 ; CSc1nc2c3c(nc(Cl)c(F)c3n1)N(C)[C@H]1COCC[C@@H](C)N21]

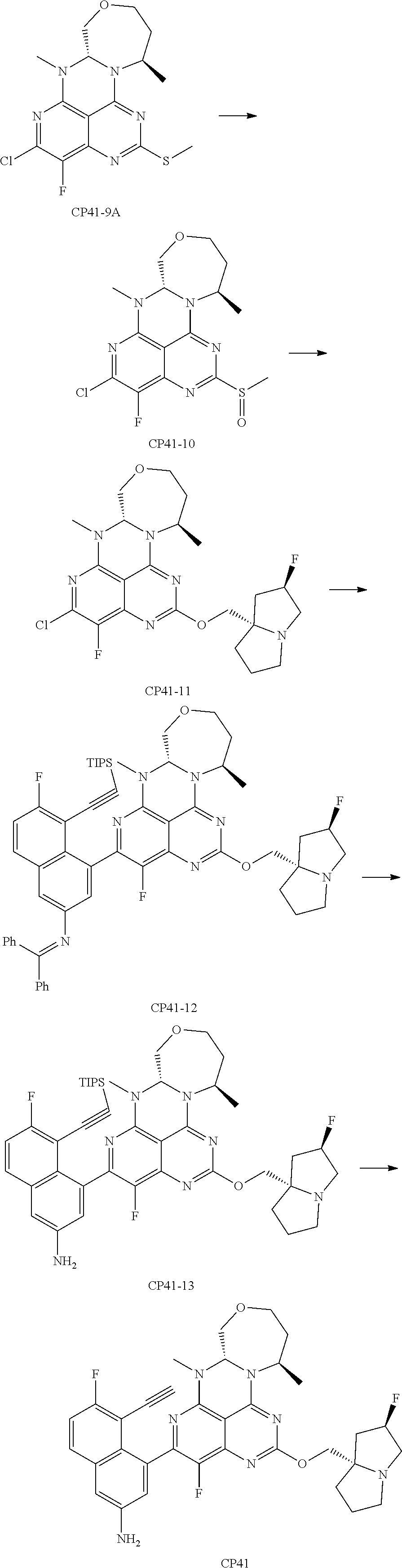

To a solution of 41-a (5539 mg) in diethyl ether (13 mL) was added NaH (405 mg) at 0° C. under N₂. The resulting mixture was stirred for 0.5 hour at RT. After cooling to 0° C., trichloroacetonitrile (4 mL) was added dropwise. The resulting mixture was stirred for 1.5 hours at 0° C., then hexane (100 mL) was added. The mixture was filtered and the filtrate was concentrated to afford CP41-1 (9.78 g).

A solution of ethyl (S)-3-hydroxybutanoate (2.6312 g) in DCM (56 mL) was stirred at RT under N₂. A solution of CP41-1 (9.78 g) in DCM (56 mL) was added dropwise. Once completion, (1S)-(+)-camphor-10-sulphonic acid (0.55 g) was added. The resulting mixture was stirred overnight at RT, quenched with sat. aq. NaHCO₃ and post processed to afford CP41-2 (7.28 g).

To a solution of CP41-2 (7.28 g) in THF (30 mL) was added LiAlH₄ (1.11 g) in portions at 0° C. The resulting mixture was stirred overnight at RT, quenched with wet Na₂SO₄ and filtered. The filtrate was concentrated and purified to afford CP41-3 (1.98 g).

To a solution of CP41-3 (1.46 g) in THF (30 mL) was added 2-bromo-1,1-dimethoxyethane (3.95 g), TBAI (0.26 g) and NaH (1.17 g), the resulting mixture was stirred at 80° C. for 16 hours, quenched by water, extracted with EA and post processed to afford CP41-4 (2.03 g).

CP41-6 was synthesized in a manner similar to CP46 with INT 6

A mixture of CP41-6 (1.39 g), CP41-4 (1.79 g) and p-TsOH·H₂O (0.78 g) in DMSO (15 mL) was stirred at 80° C. for 5 hours. The mixture was added water, extracted with EA and post processed to afford CP41-7 (2.31 g). MS (ESI, m/z): 628[M+H]⁺.

A mixture of CP41-7 (2.31 g) in TFA (15 mL) was stirred at 120° C. overnight. The mixture was concentrated. To the residue were added NaOH (aq) and MeOH and stirred for 1 hour at RT. The resulting mixture was extracted with DCM and EA and post processed to afford CP41-8 (1.06 g). MS (ESI, m/z): 388[M+H]⁺.

To a mixture of CP41-8 (1.01 g), DIEA (1033 mg) in DCM (15 mL) was added methanesulfonyl chloride (287 mg) dropwise. The resulting mixture was stirred at RT for 1 hour and then diluted with water. The organic layer was separated, dried and concentrated. The residue was dissolved in acetonitrile (15 mL). The resulting mixture was added Cs₂CO₃ (2564 mg) and stirred for 3 hours at 60° C. The resulting mixture was diluted with water and EA (30 mL) and filtered through celite. The filtrate was extracted with EA and post processed to afford CP41-9 (320 mg). MS (ESI, m/z): 370[M+H]⁺.

CP41-9 (320 mg) was separated by chiral-HPLC Gilson with the conditions: Column, CHIRAL ART Cellulose SC column (2 cm×25 cm, 5 um); mobile phase, (Hex:DCM=3:1)(0.1% DEA)/EtOH (50:50); Flowing rate: 18 mL/min. This resulted in CP41-9A (0.16 g. the first eluting isomer, Retention Time 4.933 min) and CP41-9B (the second eluting isomer, Retention Time 11.277 min).

Compound 41 (CP41, 18.2 mg, TFA salt, confirmed) was synthesized in a manner similar to CP46 with CP41-9A. MS (ESI, m/z): 630[M+H]⁺.

### Example 43

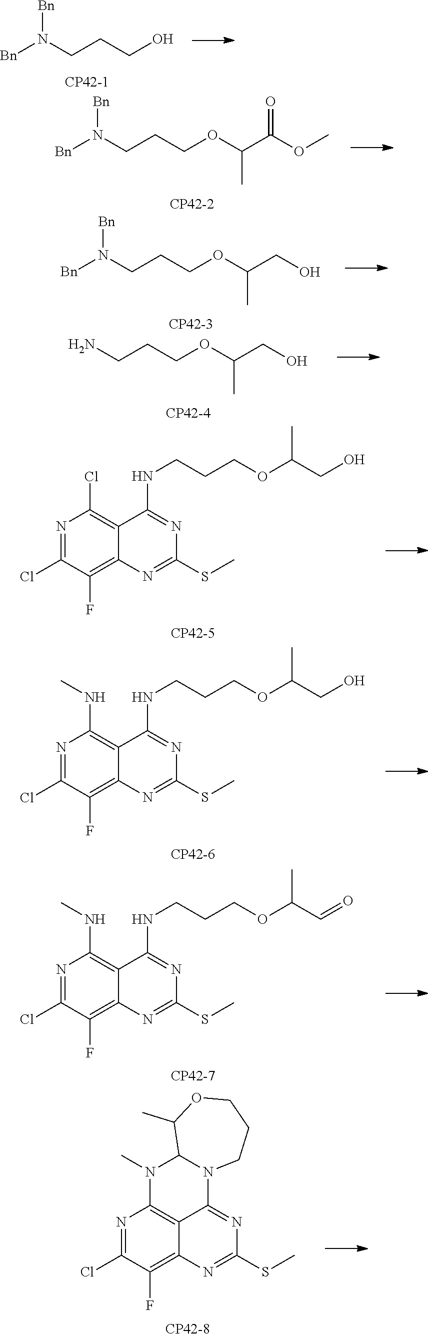

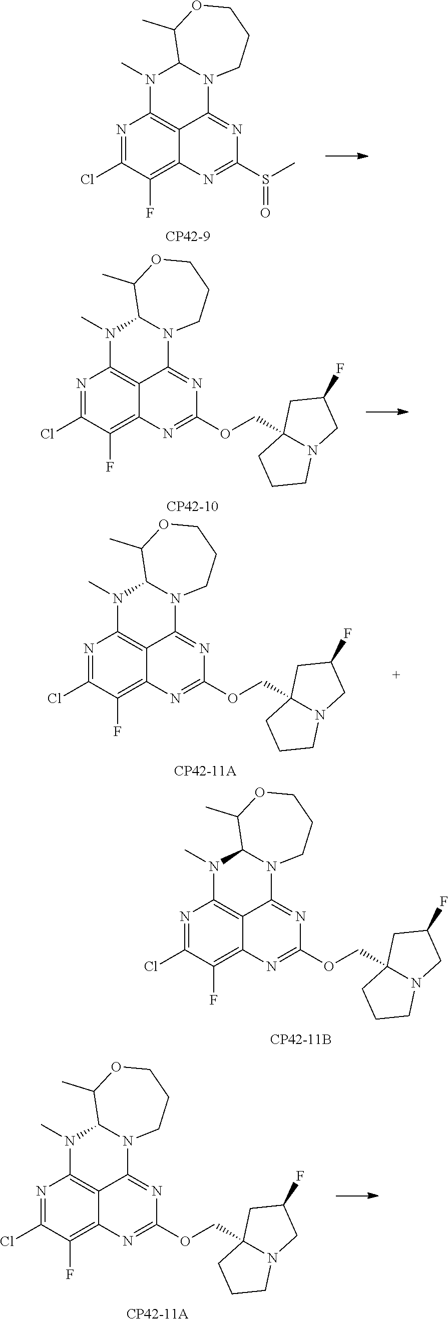

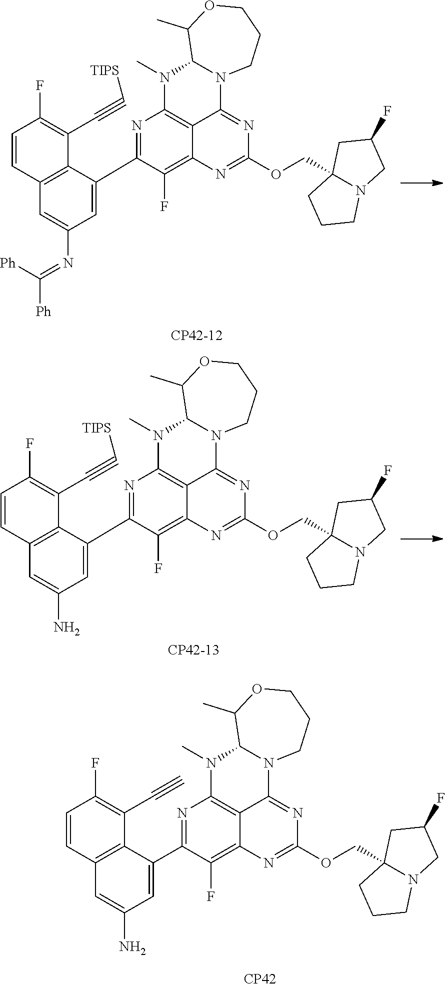

To a solution of CP42-1 (3.08 g) in dry THF (25 mL) was added sodium hydride (60% in oil, 0.59 g) under nitrogen at 0° C. and the resulting mixture was stirred at RT for 30 minutes. A solution of methyl 2-bromopropanoate (3.00 g) in dry THF (5 mL) was added to the reaction mixture and stirred at RT for 1 h. After completion, the reaction mixture was diluted with EA and saturated NH₄Cl and post processed to afford CP42-2 (4.05 g). MS: m/z: 342[M+H]⁺.

To a solution of CP42-2 (3.55 g) in THF (40 mL) was added LAH (0.45 g) under nitrogen at 0° C. and the resulting mixture was stirred at RT for 1 h, quenched with ice water (5 mL) and filtered. The filtrate was concentrated and purified to afford CP42-3 (2.11 g). MS: m/z: 314[M+1]⁺.

A solution of CP42-3 (1.94 g), Pd/C (0.54 g, 10% wt) and Pd(OH)₂/C (0.54 g, 10%/wt) in MeOH (60 mL) was stirred at RT for 5 hours under H₂. The reaction mixture was filtered and the filtrate was concentrated to afford CP42-4 (693 mg). MS: m/z: 134[M+H]⁺.

CP42-10 was synthesized in a manner similar to CP40-11 with CP42-4.

CP42-10 (270 mg) was separated by Prep-HPLC-Gilson with the conditions: Column, CHIRAL ART Cellulose-SC column (2 cm×25 cm, 5 um; Mobile phase, (HeX:DcM=3:1)(0.1% DEA)/EtOH (50:50). Flowing rate: 18 ml/min. This resulted in CP42-11A (120 mg, the first eluting isomer, Retention time: 5.557 min) and CP42-11B (116 mg, the second eluting isomer. Retention time: 6.677 min).

Compound 42 (CP42, confirmed) was synthesized in a manner similar to CP46 with CP42-11A. MS: m/z: 630[M+H]⁺.

### Example 44

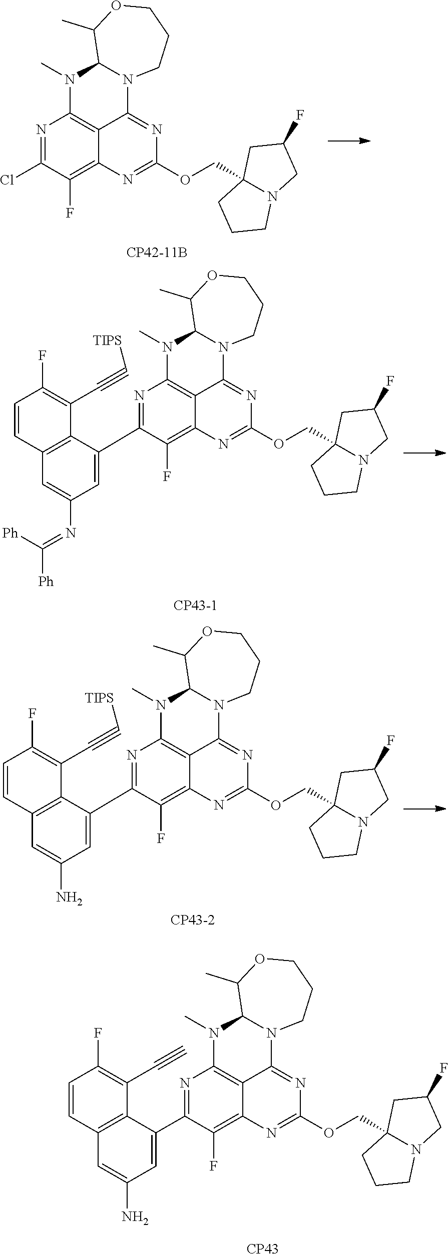

Compound 43 (CP43, confirmed) was synthesized in a manner similar to CP46 with CP42-11B. MS: m/z: 630[M+H]⁺.

### Example 45

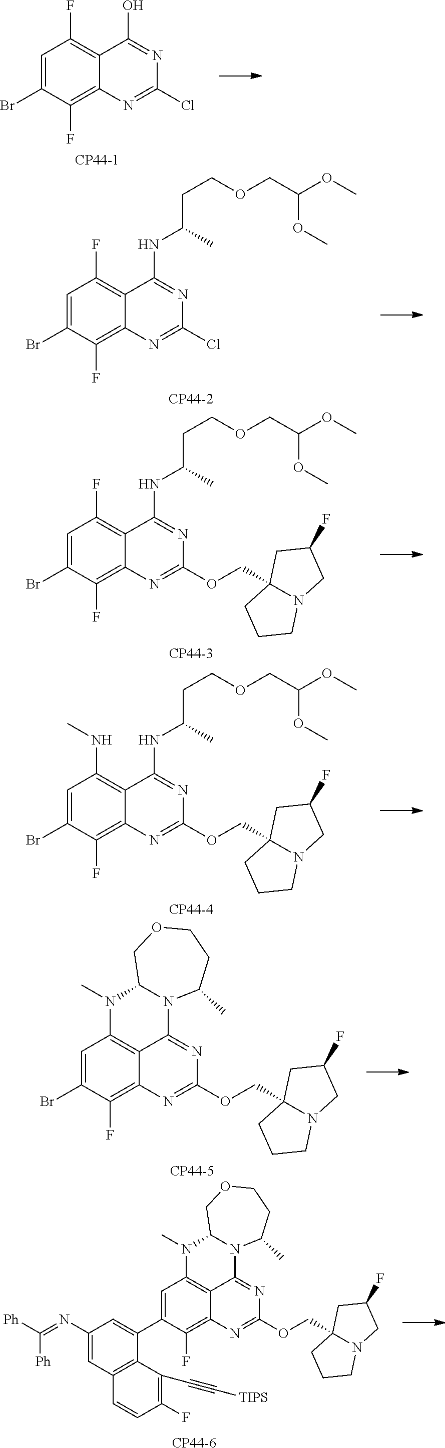

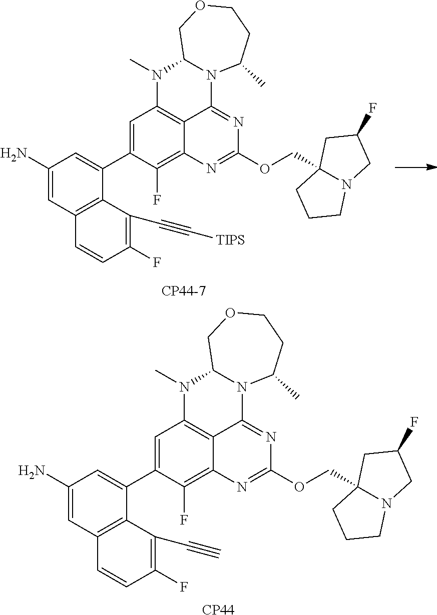

To a solution of CP44-1 (517 mg), DIEA (634 mg) in toluene (10 mL) was added POCl₃ (0.24 ml). The reaction mixture was stirred at 80° C. for 1 h and concentrated to afford the residue. To a solution of CP36-4 (338 mg), DIEA (1 ml) in DCM (10 mL) was added the residue in DCM (10 ml) at 0° C. The reaction was stirred at room temperature for 0.5 h, diluted with water, extracted with DCM and post processed to afford CP44-2 (418 mg). MS m/z: 454[M+H]⁺.

To a solution of CP44-2 (280 mg) and INT14 (217 mg) in 1,4-dioxane (2.5 mL) was added DIEA (249 mg). The mixture was stirred at 100° C. for 44 h, quenched with water (30 mL), extracted with EA (30 mL) and post processed to afford CP44-3 (226 mg). MS m/z: 577[M+H]⁺.

To a solution of CP44-3 (121 mg) and methylamine hydrochloride (203 mg) in DMAc (2 mL) was added Cs₂CO₃ (1311 mg). The mixture was stirred at 110° C. for 21 h, quenched with water (30 mL), extracted with EA (30 mL) and post processed to afford CP44-4 (120 mg). MS: m/z: 587[M+H]⁺.

Compound 44 (CP44, confirmed) was synthesized in a manner similar to CP46 with CP44-4. MS: m/z: 629[M+H]⁺.

### Example 46

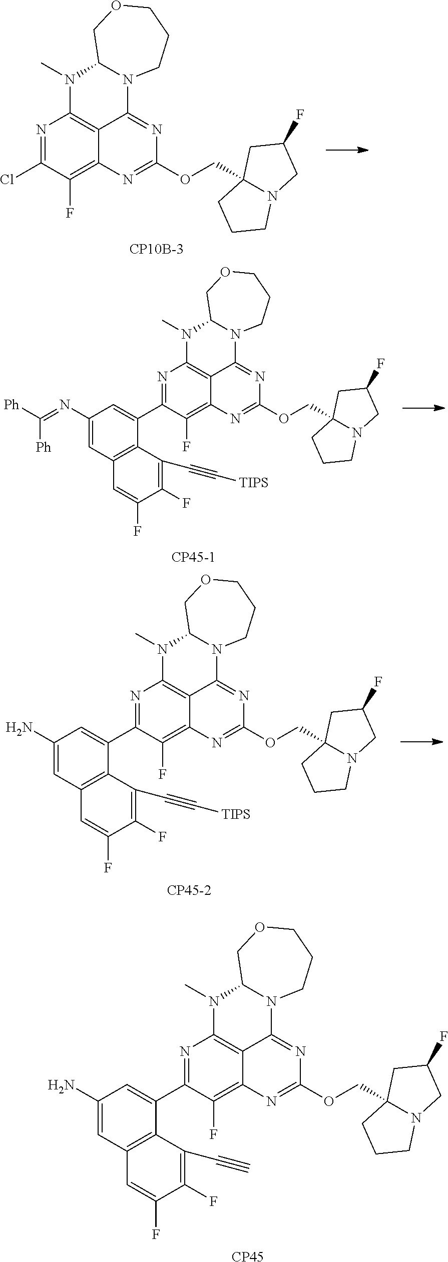

Compound 45 (CP45, confirmed) was synthesized in a manner similar to CP46 with CP10B-3 and INT10. MS: m/z: 634[M+H]⁺.

### Example 47

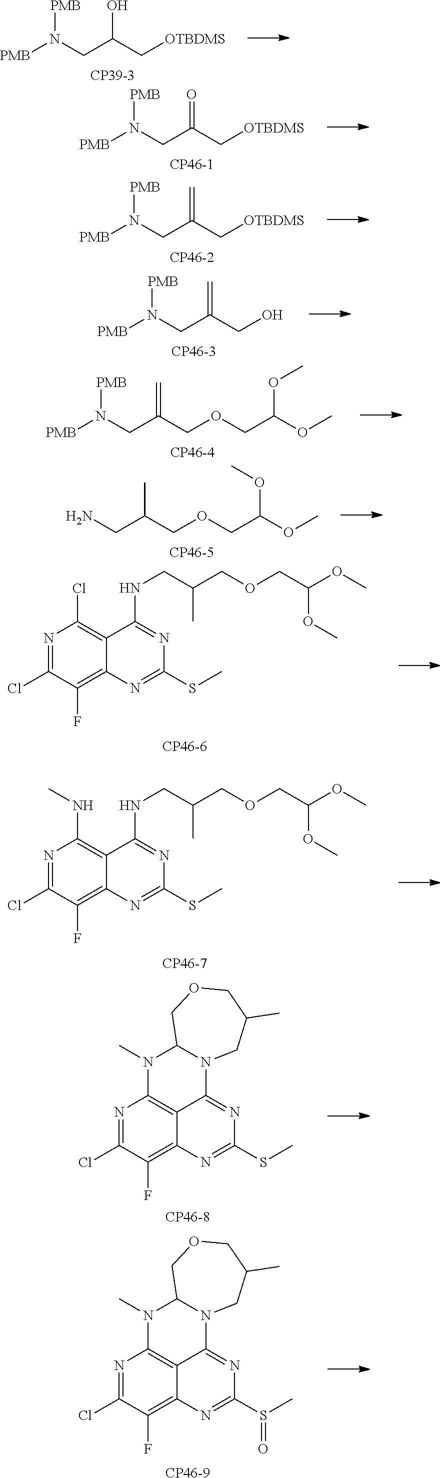

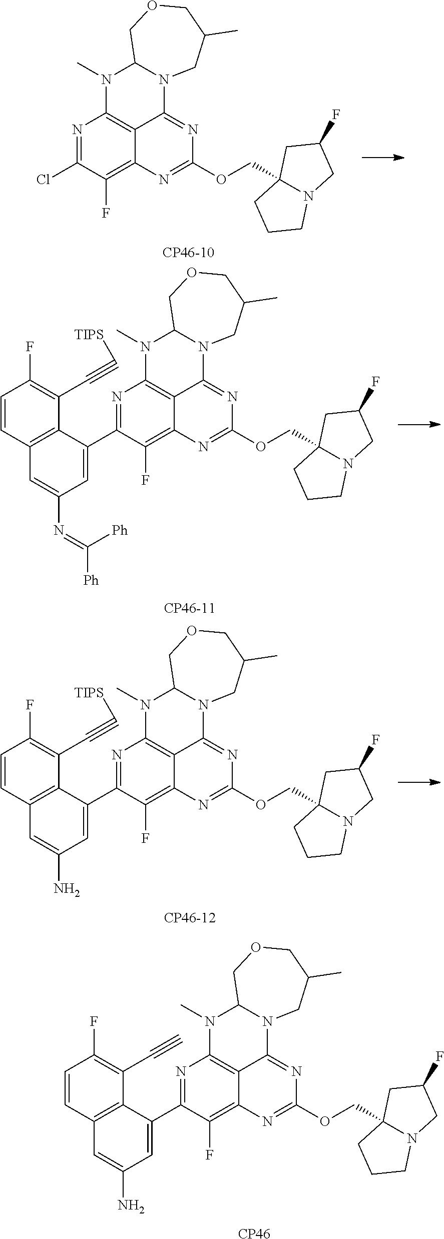

To a solution of CP39-3 (9.86 g) in DMSO (80 mL), dichloromethane (40 mL) was added TEA (19.84 g) and pyridine sulfur trioxide (14.15 g) at 0° C. The reaction mixture was stirred at RT for 16 hours. The mixture was diluted with water, extracted with EA and post processed to afford CP46-1 (10.79 g). MS: m/z: 444[M+H]⁺.

To a solution of methyltriphenylphosphonium bromide (12.68 g) in THF (100 mL) was added potassium tert-butoxide (4.04 g) at 0° C. After stirring for 1 h at RT, to the mixture was added CP46-1 (10.45 g). The reaction mixture was stirred at RT for 16 hours. The mixture was diluted with water, extracted with EA and post processed to afford CP46-2 (7.72 g). MS: m/z: 442[M+H]⁺.

To a solution of CP46-2 (7.70 g) in DMF (50 mL), CsF (8.12 g) was added. The reaction mixture was stirred for 16 hours at RT. The mixture was diluted with water, extracted with EA and post processed to afford CP46-3 (5.17 g). MS: m/z: 328[M+H]⁺.

To a solution of CP46-3 (3.78 g) in THF (50 mL) was added sodium hydride (60% in oil, 1.84 g) at 0° C. After stirring for 1 h, to the mixture were added DMAP (0.13 g), TBAI (0.63 g) and bromoacetaldehyde dimethyl acetal (6.59 g). The reaction mixture was stirred at 70° C. for 23 hours, quenched with water, extracted with EA and post processed to afford CP46-4 (4.45 g). MS: m/z: 416[M+H]⁺.

To a solution of CP46-4 (3.18 g) in MeOH (100 mL) was added Pd(OH)₂/C (2.96 g). The reaction mixture was stirred at RT for 16 hours under H₂. The mixture was filtered and the filtrate was concentrated to afford CP46-5 (1.5 g). MS: m/z: 178[M+H]⁺.

Compound 46 (CP46, 31.8 mg, confirmed) was synthesized in a manner similar to CP39 with CP46-5 and INT6. MS: m/z: 630[M+H]⁺.

### Example 48

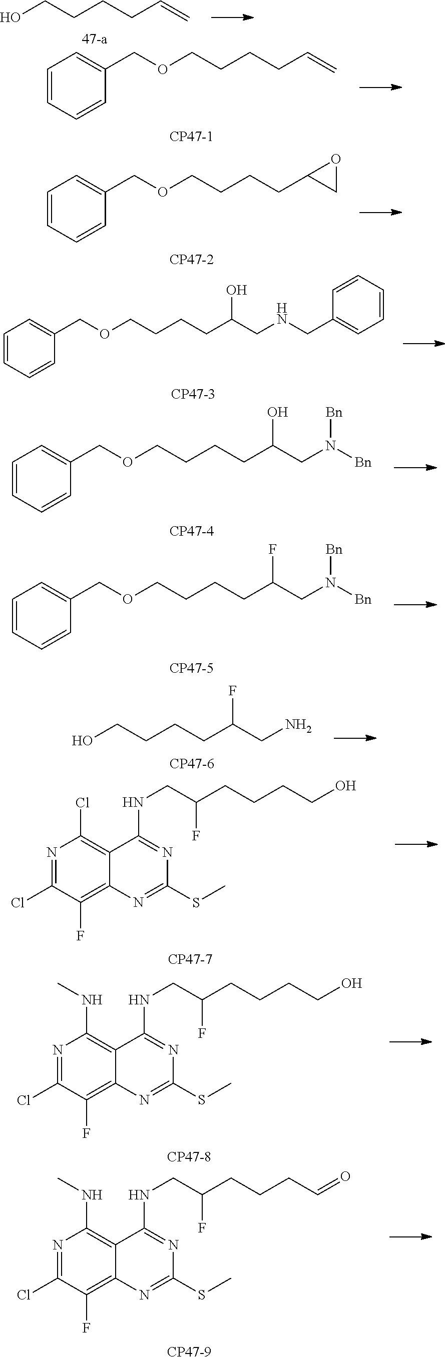

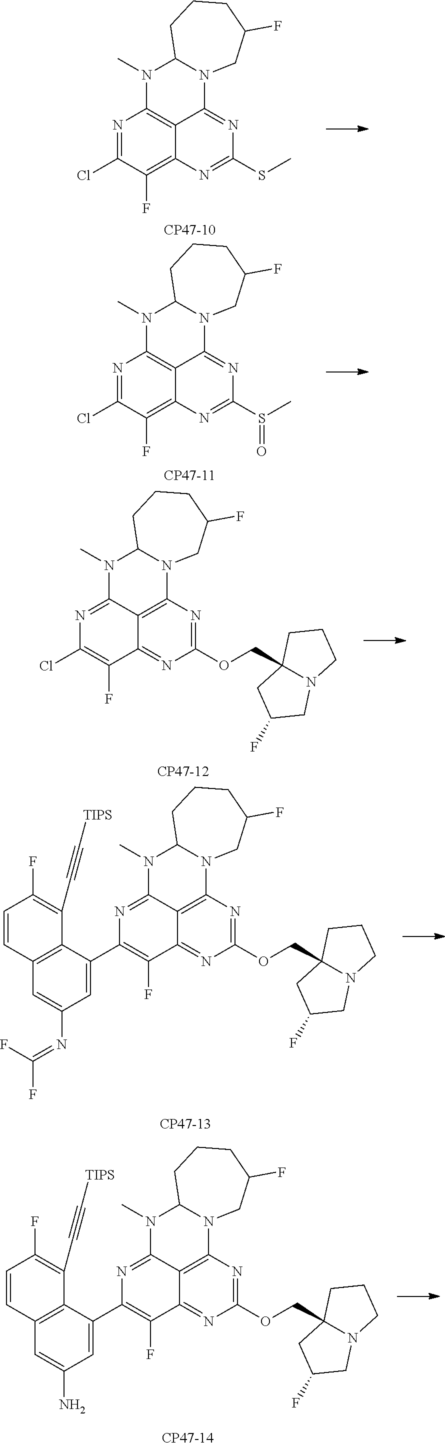

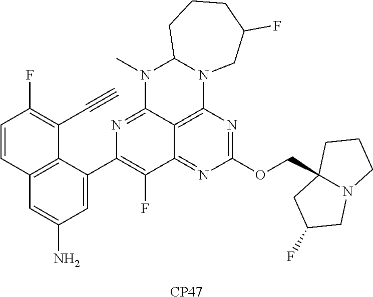

A solution of 47-a (20.04 g) in THF (200 mL) was cooled to 0° C., NaH (16.27 g), TBAI (4.45 g), DMAP (1.30 g), BnBr (43.06 g) was added to the solution. The reaction mixture was stirred for 16 h at RT, quenched with water, extracted with EA and post processed to afford CP47-1 (42.71 g).

To a solution of CP47-1 (18.64 g) in DCM (200 mL) was added m-CPBA (18.45 g) at RT and the resulting mixture was stirred for 16 h, filtrated, concentrated, and purified to afford CP47-2 (10.93 g). MS: 207[M+1]⁺.

To a solution of CP47-2 (10.93 g) in MeOH (200 mL) was added phenylmethanamine (17.15 g). The reaction mixture was stirred at 130° C. for 3 hours under N₂. The mixture was allowed to cool to RT. The mixture was concentrated and purified to afford CP47-3 (10.65 g). MS: 314[M+1]⁺.

A solution of CP47-3 (10.65 g), K₂CO₃ (14.96 g), TBAI (0.84 g), BrBn (7.78 g) in ACN (120 mL) was stirred at RT for 16 h. The mixture was concentrated and purified to afford CP47-4 (8.33 g). MS: 404[M+1]⁺.

To a solution of CP47-4 (8.33 g) in DCM (100 mL), DAST (4.72 g) was added. The reaction mixture was stirred for 3 hours at 0° C. The solution was diluted with sat.NaHCO₃, extracted with EA and post processed to afford CP47-5 (5.65 g). MS: 406[M+1]⁺.

To a solution of CP47-5 (5.65 g) in methanol (80 mL) was added Pd(OH)₂/C (1.63 g) at H₂. The reaction mixture was stirred at 60° C. for 16 hours. The resulting mixture was filtered and the filtrate was concentrated to afford CP47-6 (1.78 g). MS: 136[M+1]⁺.

Compound 47 (CP47, 12.8 mg, confirmed) was synthesized in a manner similar to CP40 with CP47-6. MS: 632[M+H]⁺.

### Example 49

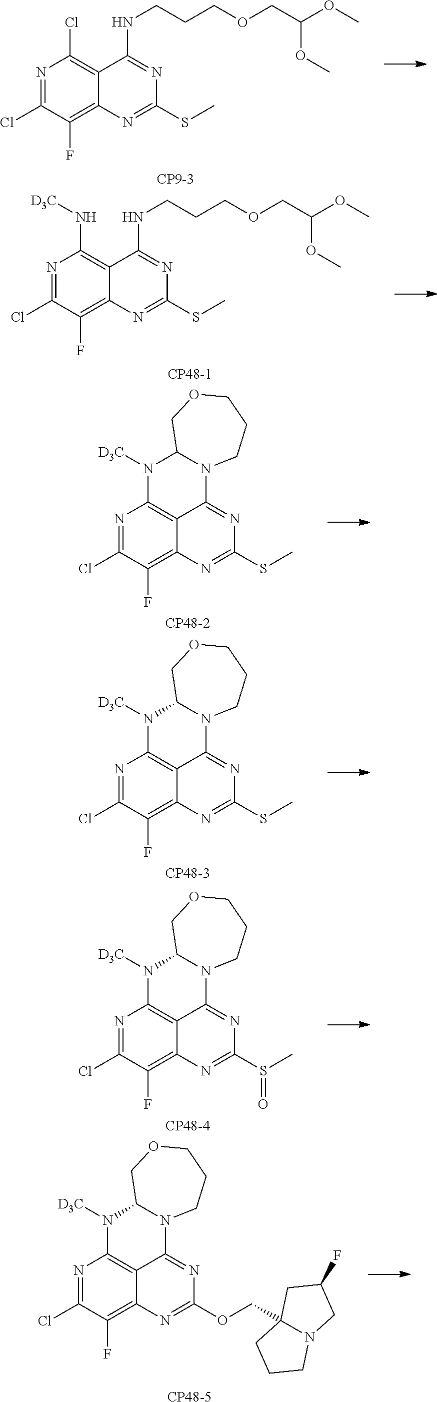

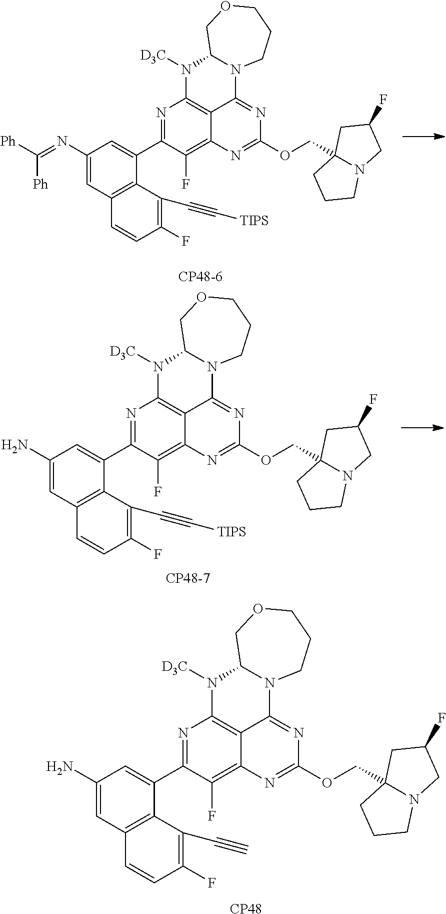

A solution of CP9-3 (1.058 g), Cs₂CO₃ (3001 mg) and CD₃NH₂·HCl (510 mg) in DMF (15 mL) was stirred at 1000(C for 4 hours. The reaction mixture was cooled to RT. The residue was diluted with water, extracted with EA and post processed to afford the crude CP48-1 which was used in the next step directly. MS (ESI, m/z): 423[M+H]⁺.

CP49-2 was synthesized in a manner similar to CP46-8 with CP48-1.

CP48-2 (690 mg) was separated by Prep-HPLC-Gilson with the conditions: Column, CHIRAL ART Cellulose-SC column (2 cm×25 cm, 5 um); mobile phase, (Hex:DCM=3:1)/EtOH=50:50; Flowing rate: 18 ml/min, 220 nm. This resulted in CP48-3 (the second eluting isomer, Retention time: 11.7 min.

Compound 48 (CP48, confirmed) was synthesized in a manner similar to CP46 with CP48-3. MS (ESI, m/z): 619 [M+H]⁺.

### Example 50

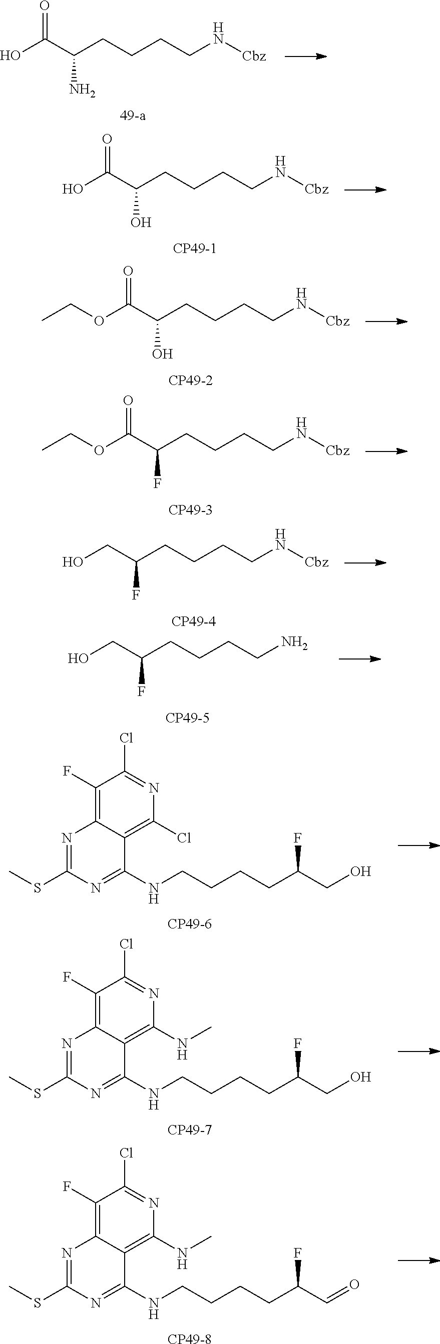

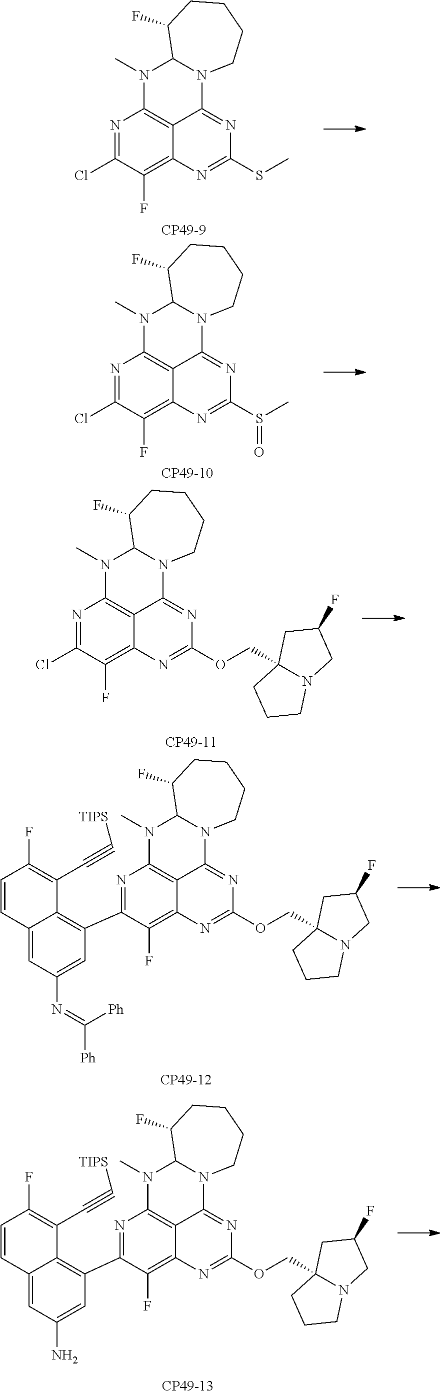

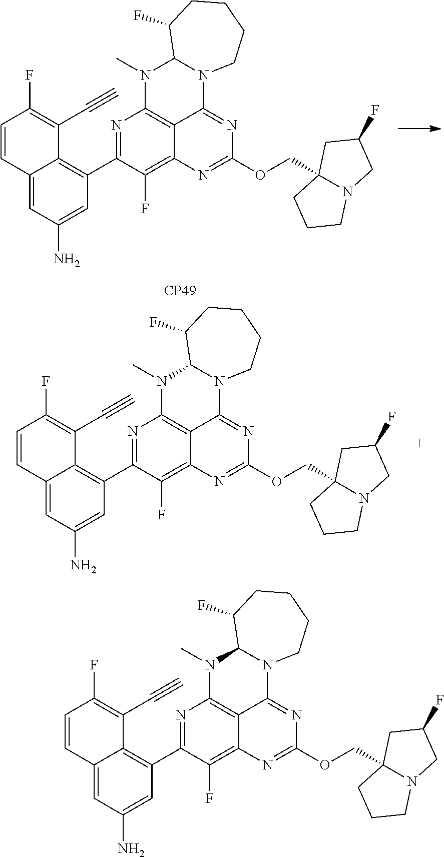

CP49 was synthesized in a manner similar to CP40 with CP49-a. MS: m/z: 632[M+1]⁺.

CP49 (50 mg) was separated by Prep-HPLC (Agela Durashell C18, 30 mm×250 mm, 10 um, A: 0.05% NH₃·H₂O, B: CH₃CN, Gradient: 25% B to 58% B in 35 min at a flow rate of 40 mL/min, 224 nm). This resulted in Compound 49A (CP49A, the first eluting isomer, Retention time: 30.8-33.39 min) and Compound 49B (CP49B, the second eluting isomer. Retention time: 33.64-35.75 min) MS: m/z: 632[M+1]⁺.

### Example 51

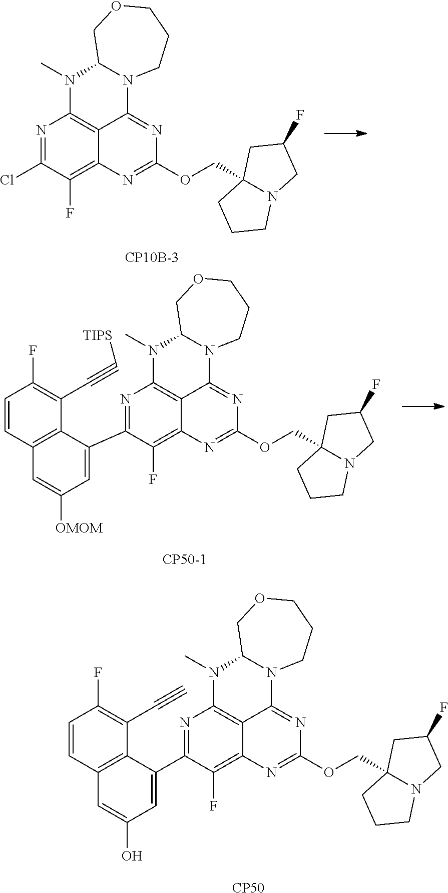

Compound 50 (CP50, confirmed) was synthesized in a manner similar to CP2 with CP10B-3. LCMS: m/z: 617[M+H]⁺.

¹HNMR (400 MHz, DMSO-d₆) δ 11.16-10.82 (m, 1H), 8.02-7.90 (m, 1H), 7.51-7.41 (m, 1H), 7.36 (d, J=1.9 Hz, 1H), 7.22-7.10 (m, 1H), 5.58 (d, J=53.2 Hz, 1H), 5.38-5.26 (m, 1H), 4.65-4.51 (m, 2H), 4.51-4.41 (m, 1H), 4.13-3.94 (m, 2H), 3.94-3.68 (m, 3H), 3.64-3.49 (m, 1H), 3.45-3.22 (m, 3H), 3.12-2.93 (m, 3H), 2.63-2.44 (m, 3H), 2.41-2.10 (m, 4H), 2.09-1.89 (m, 2H).

### Example 52

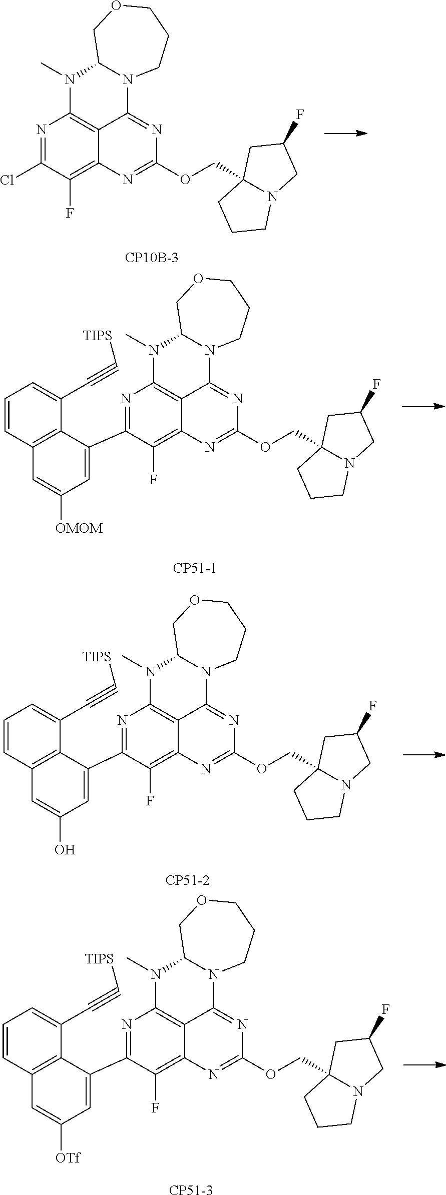

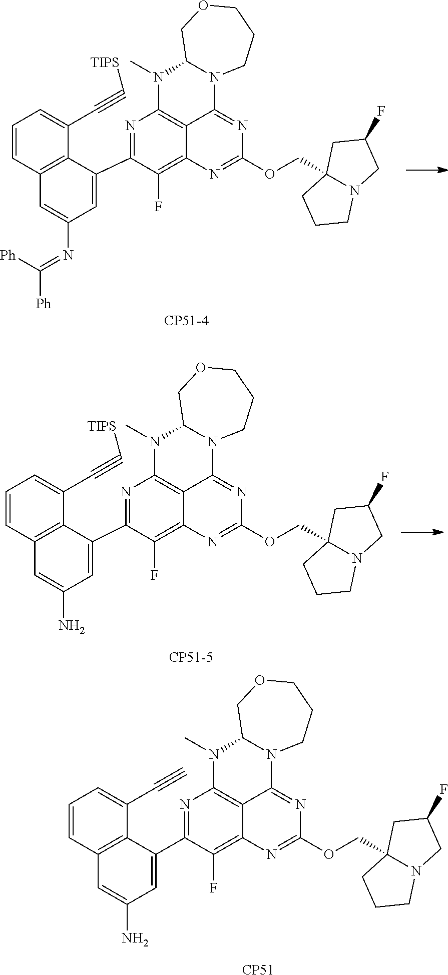

Compound 51 (CP51, confirmed) was synthesized in a manner similar to CP1 with CP10B-3 and INTL. MS: m/z: 598[M+H]⁺.

¹HNMR (400 MH-z, DMSO-d₆) 57.79-7.68 (m, 1H), 7.31-7.29 (m, 2H), 7.18-7.08 (m, 1H), 6.97-6.93 (m, 1H), 5.32 (s, 2H), 4.19-4.16 (m, 2H), 3.88-3.80 (m, 3H), 3.53-3.50 (m, 2H), 3.35-3.27 (m, 2H), 3.02 (s, 3H), 2.67-2.51 (m, 4H), 2.33-2.31 (m, 3H), 2.22-2.18 (m, 4H), 2.13-2.04 (m, 3H).

### Example 53

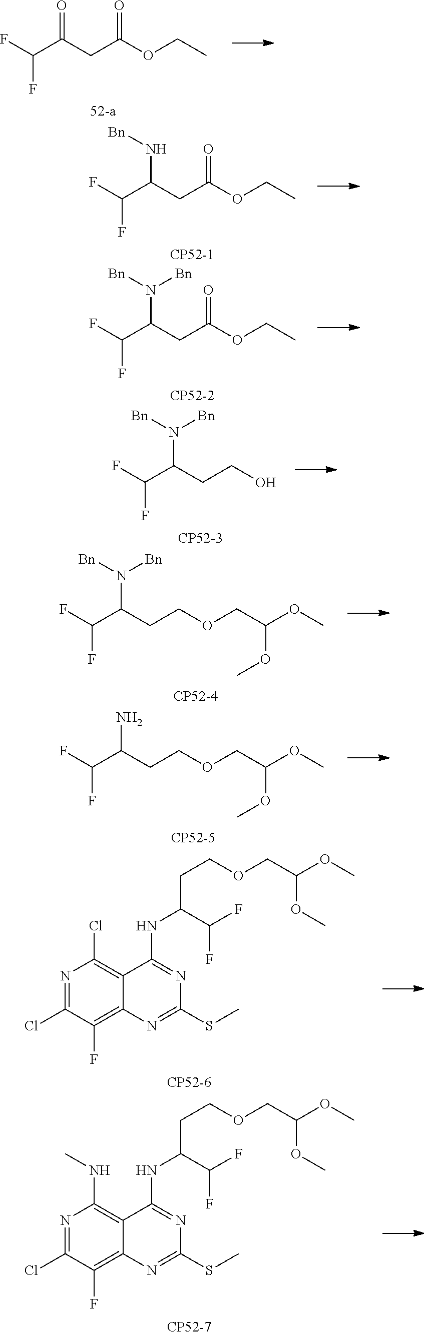

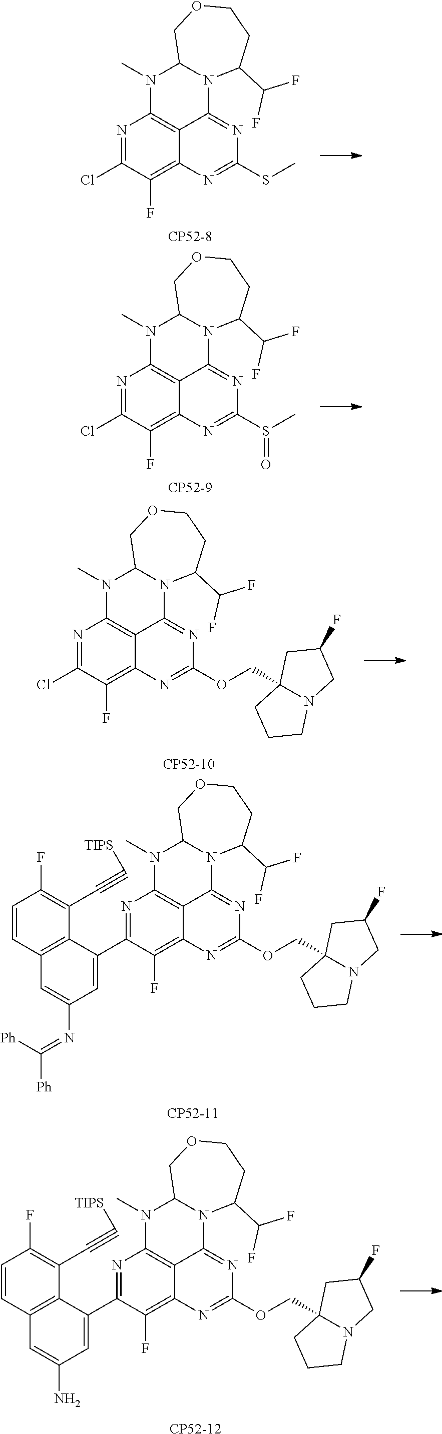

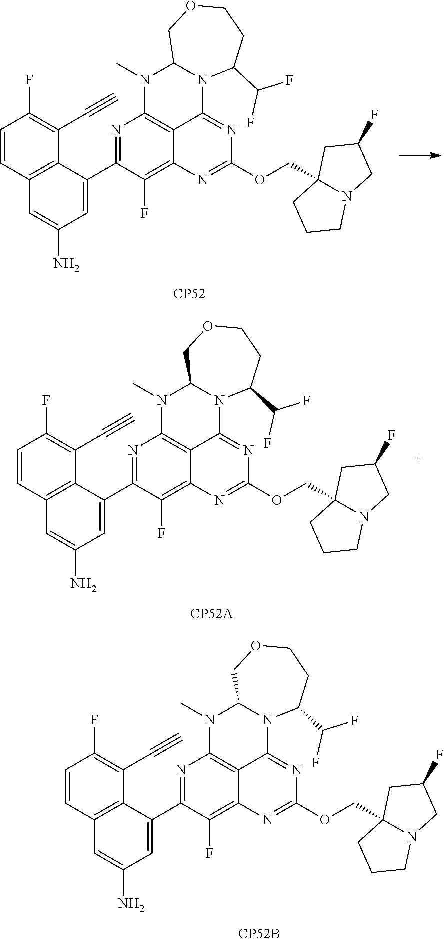

CP52 was synthesized in a manner similar to CP94 with CP52-a and separated by Prep-HPLC-Gilson with the conditions: Column. CHIRAL ART Cellulose-SA column (2 cm×25 cm, 5 um); mobile phase, Hex (0.1% DEA)/EtOH (50:50); Flowing rate: 20 mL/min. This resulted in Compound 52A (CP52A, 7.8 mg, the first eluting isomer, Retention time: 5.227 min, confirmed) and Compound 52B (CP52B, 10.7 mg, the second eluting isomer, Retention time: 6.157 min. confirmed). LCMS: m/z: 666[M+H]⁺.

### Example 54

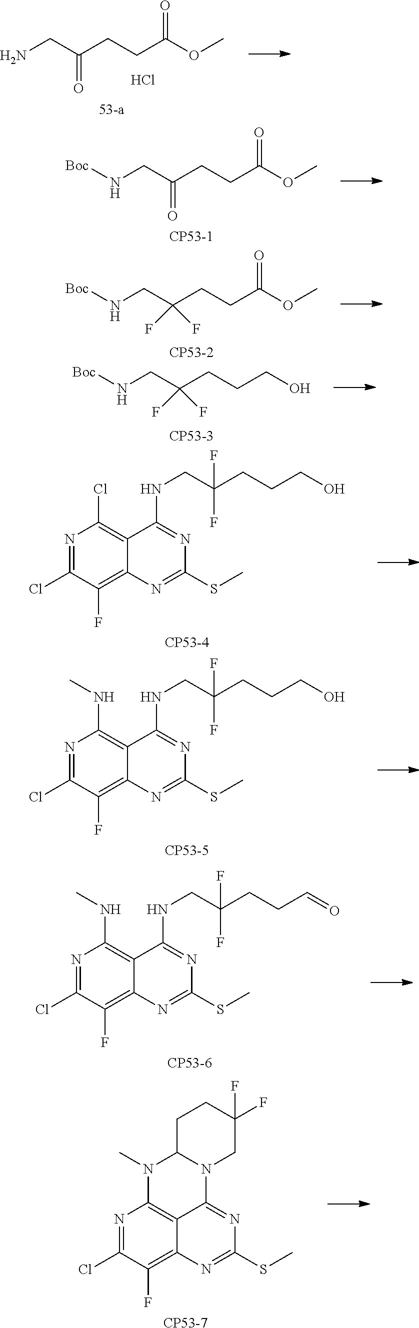

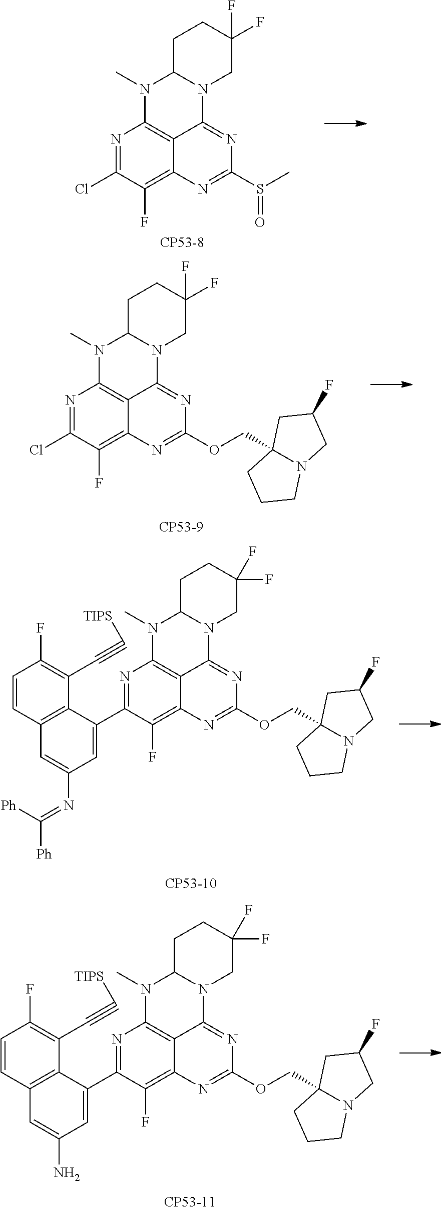

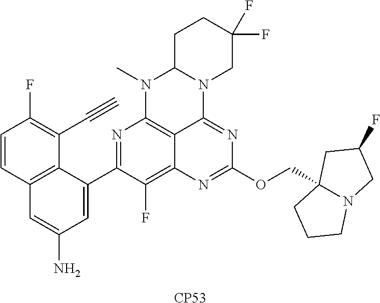

To a mixture of 53-a (3.97 g) in THF (50 mL) was added DIEA (8.47 g) and (Boc)₂O (5.20 g). The reaction mixture was stirred at RT for 3 hours, diluted with EA and water and post processed to afford CP53-1 (4.17 g). MS: m/z: 246[M+H]⁺.

To a solution of CP53-1 (4.17 g) in DCM (50 mL) was added DAST (23.90 g). The reaction mixture was stirred at 40° C. for 18 hours. Then sat. aq. Na₂CO₃ (100 mL) was added at 0° C. and post processed to afford CP53-2 (2.73 g). MS: m/z: 268[M+H]⁺.

To a solution of CP53-2 (2.52 g) in THF (20 mL) was added LAH (0.56 g) in batches at −10° C. The reaction mixture was stirred at RT for 0.5 h, quenched with water (1 mL), aq. NaOH (0.6 mL, 15% w/w) and water (3 mL). The mixture was filtered and the filtrate was concentrated and purified to afford CP53-3 (1589 mg). MS: m/z: 240[M+H]⁺.

To a solution of CP53-3 (0.77 g) in acetonitrile (9 mL) was added HCl (3 mL, 4 mol/mL in dioxane) at RT and the resulting mixture was stirred at RT for 2 hours and then concentrated to afford the residue A. To a solution of INT6 (889 mg), DIEA (1232 mg) in toluene (10 mL) was added POCl₃ (0.5 ml). The reaction mixture was stirred at 80° C. for 2 hours and concentrated to afford the residue B. To a solution of the residue A and DIEA (2 mL) in DCM (10 mL) was added the residue B in DCM (10 mL). The reaction mixture was stirred at RT for 1.5 hours, diluted with water, extracted with EA and post processed to afford CP53-4 (465 mg). MS: m/z: 401 [M+H]⁺.

Compound 53 (CP53, confirmed) was synthesized in a manner similar to CP47 with CP53-4. MS m/z: 636[M+H]⁺.

### Example 55

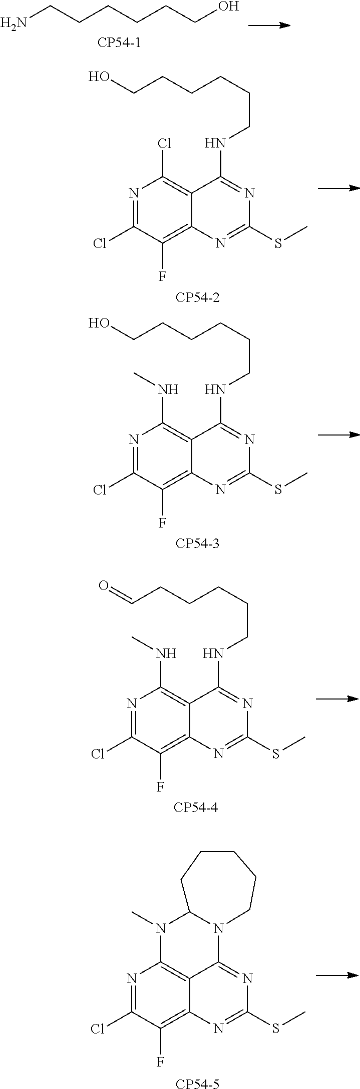

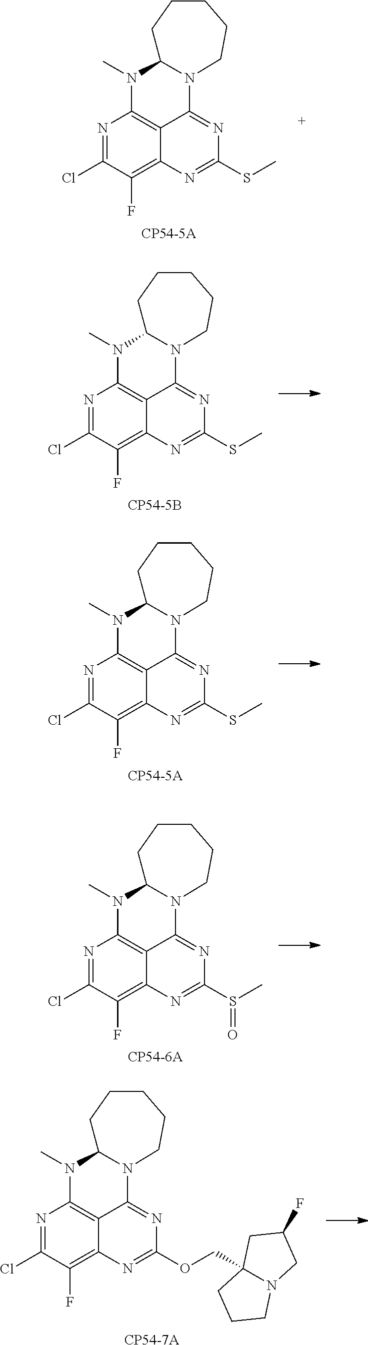

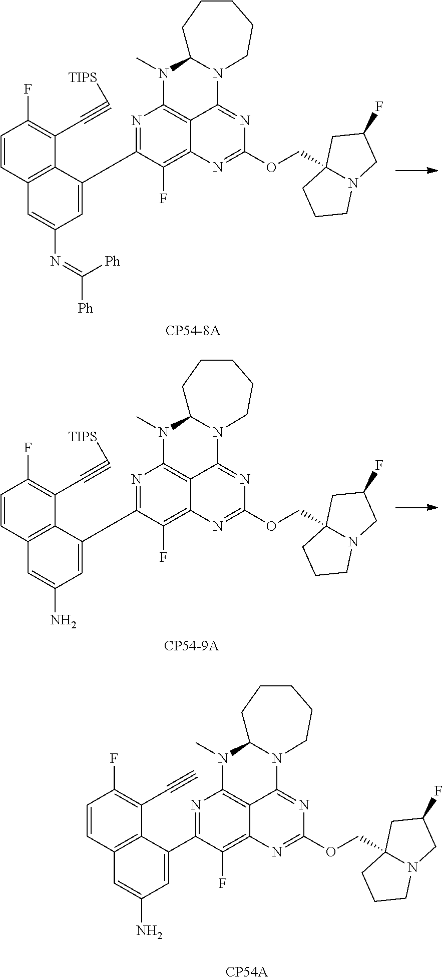

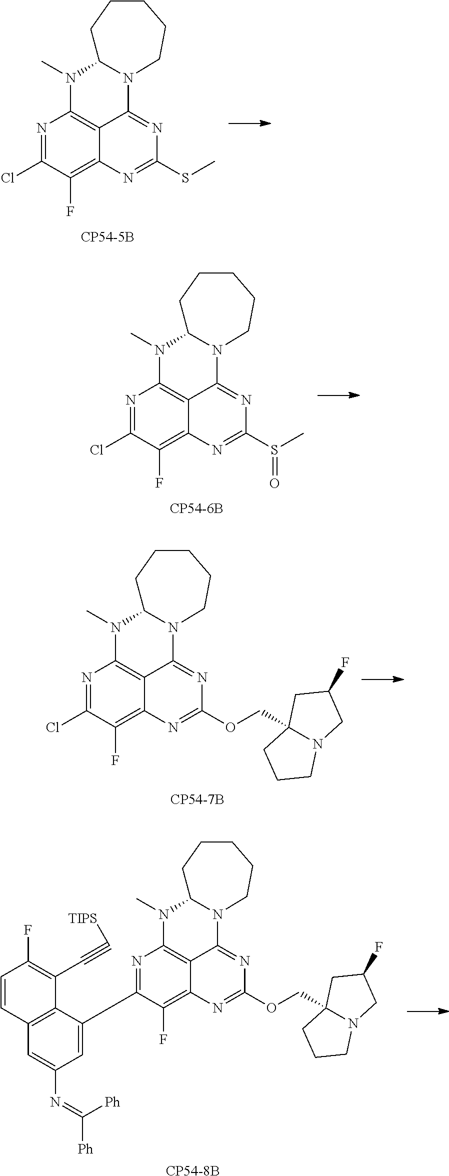

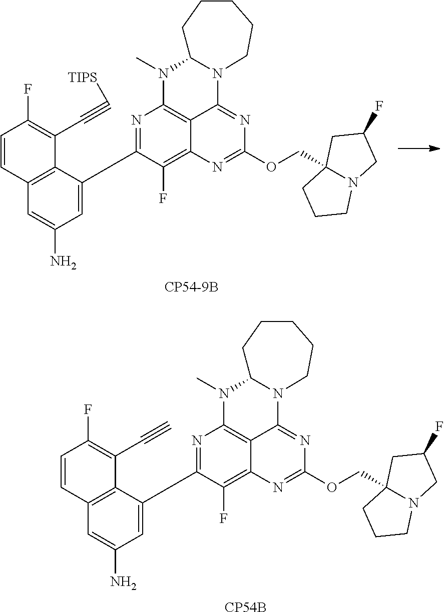

CP54-5 was synthesized in a manner similar to CP47-10 with CP54-1.

CP54-5 (133 mg) was separated by Prep-HPLC-Gilson with the conditions: Column, CHIRAL ART Cellulose-SC column (2 cm×25 cm, 5 um); mobile phase, (Hex:DCM=3:1)(0.1% DEA)/EtOH (50:50); Flow rate: 17 mL/min. This resulted in CP54-5A (53 mg, the first eluting isomer, Retention time: 5.923 min) and CP54-5B (52 mg, the second eluting isomer, Retention time: 8.129 min).

Compound 54A (CP54A, confirmed) was synthesized in a manner similar to CP47 with CP54-5A. MS: m/z: 614[M+H]⁺.

Compound 54B (CP54B, confirmed) was synthesized in a manner similar to CP47 with CP54-5B. MS: m/z: 614[M+H]⁺.

### Example 56

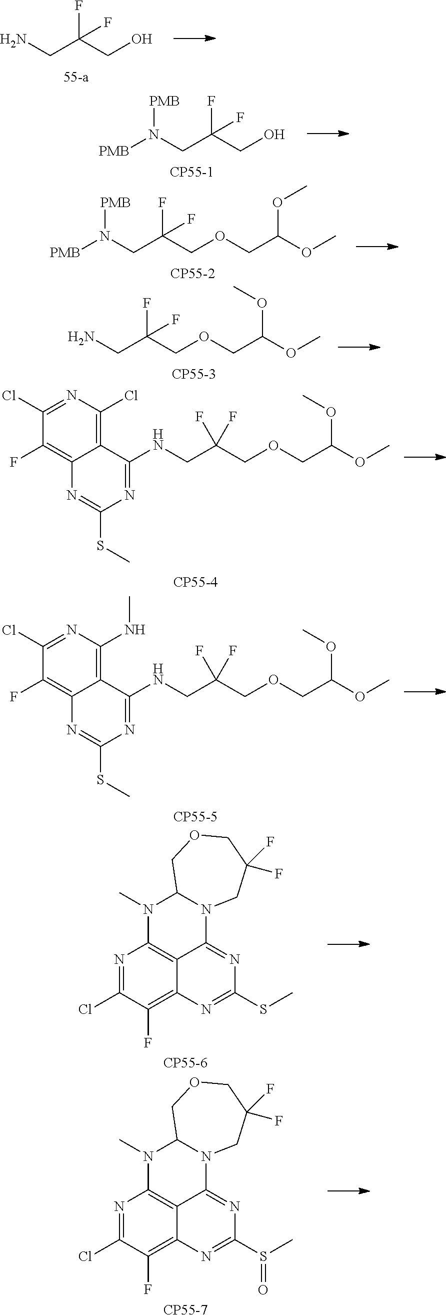

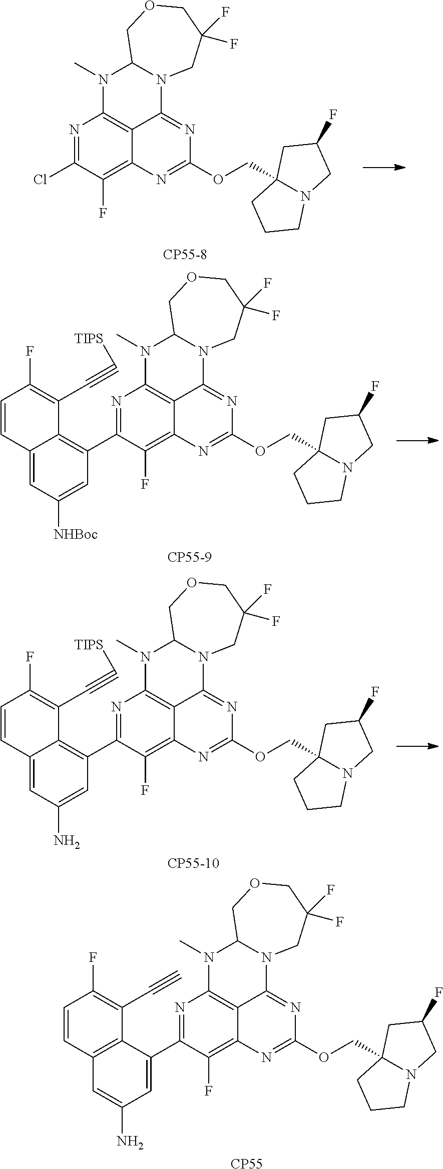

A solution of 55-a (1.58 g), DIEA (4.63 g), 4-methoxybenzylchloride (5.04 g) in DCM (20 mL) was stirred at RT for 18 h, quenched by water, extracted with DCM and post processed to afford CP55-1 (1.8874 g). MS: m/z: 352[M+H]⁺.

A solution of CP55-1 (2.7114 g) in THF (30 mL) was added NaH (1.6382 g, 60% h purity) at RT under N₂. The reaction mixture was stirred for 1 hour. The mixture was added DMAP (217 mg), TBAI (676 mg) and bromoacetaldehyde dimethyl acetal (5175 mg). The reaction mixture was stirred at 70° C. for 17 hours under N₂, quenched with water, extracted with EA and post processed to afford CP55-2 (2.8029 g, 70% purity). MS: m/z: 440[M+H]⁺.

Compound 55 (CP55, confirmed) was synthesized in a manner similar to CP46 with CP55-2. MS: m/z: 652[M+H]⁺.

### Example 57

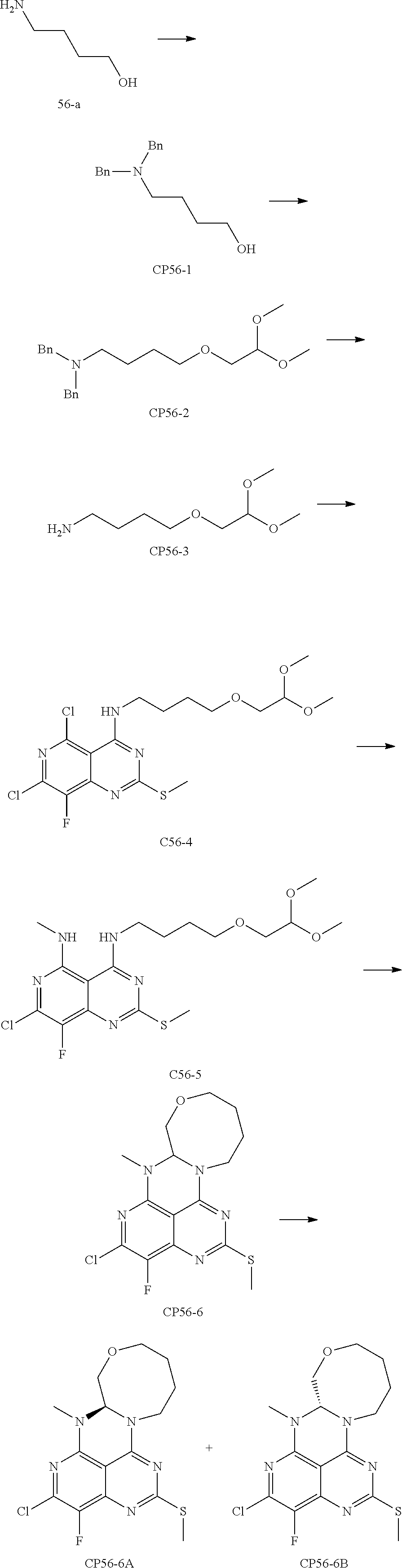

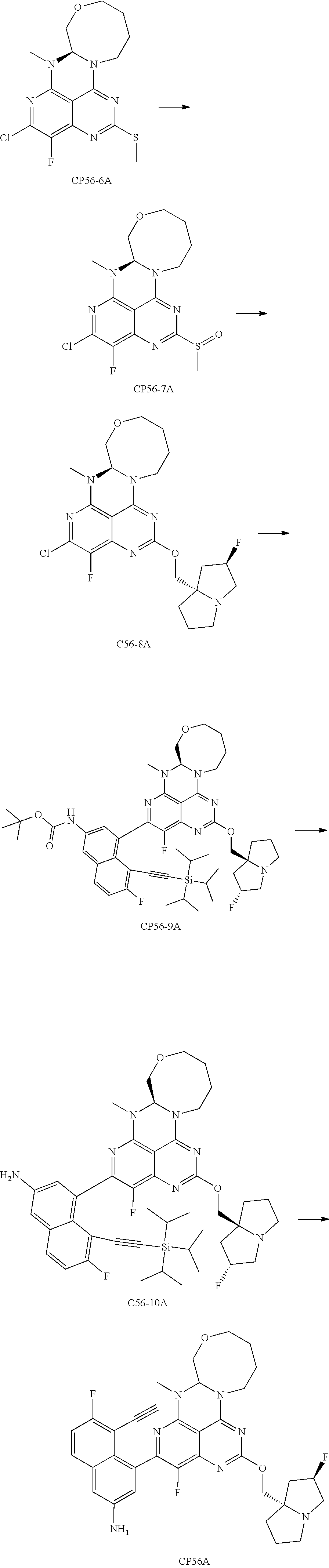

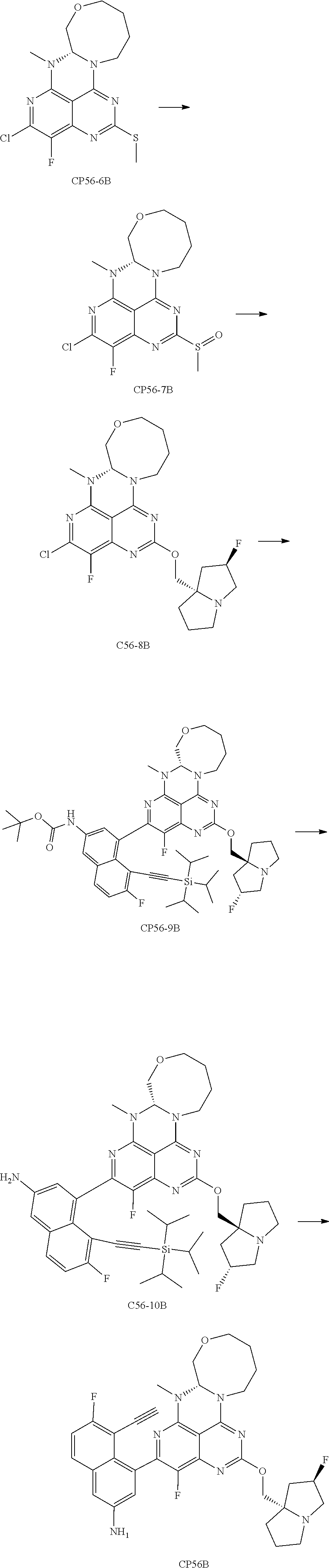

A solution of 56-a (5.03 g), TBAI (1.30 g), K₂CO₃ (24.83 g) and BrBn (24.19 g) was stirred at RT for 16 h. The mixture was concentrated and purified to afford CP56-1 (9.2 g). MS: m/z: 270[M+H]⁺.

To a solution of CP56-1 (8.8 g) in THF (100 mL) was added NaH (5.41 g, 60% purity). The reaction mixture was stirred at RT for 1 h. The reaction mixture was added DMAP (0.39 g), TBAI (1.24 g) and bromoacetaldehyde dimethyl acetal (17.72 g). The resulting mixture was stirred at 75° C. for 16 h, quenched with ice water, extracted with EA and post processed to afford CP56-2 (9.74 g). MS: m/z: 358[M+H]⁺.

To a solution of CP56-2 (9.74 g) in methanol (100 mL) was added Pd/C (2.8995 g), Pd(OH)₂/C (2.0 g) at H₂. The reaction mixture was stirred at RT for 16 hours, filtered and the filtrate was concentrated to afford CP56-3 (4.65 g). MS: m/z: 178[M+H]⁺.

CP56-6 was synthesized in a manner similar to CP46-8 with CP56-3.

CP56-6 (1.32 g) was separated by Prep-HPLC-Gilson with the conditions: Column, CHIRAL ART Cellulose-SC column (2 cm×25 cm, 5 um); mobile phase, (HeX:DcM=3:1)(0.1% DEA)/EtOH (50:50); Flowing rate: 17 ml/min. This result in CP56-6B (623 mg, the first eluting isomer, Retention time: 6.69 min) and CP56-6A (0.61 g, the second eluting isomer, Retention time: 8.708 min). MS: m/z: 370[M+H]⁺.

Compound 56A (CP56A, confirmed) was synthesized in a manner similar to CP46 with CP56-6A.

Compound 56B (CP56B, confirmed) was synthesized in a manner similar to CP46 with CP56-6B. MS: m/z: 630[M+H]⁺.

### Example 58

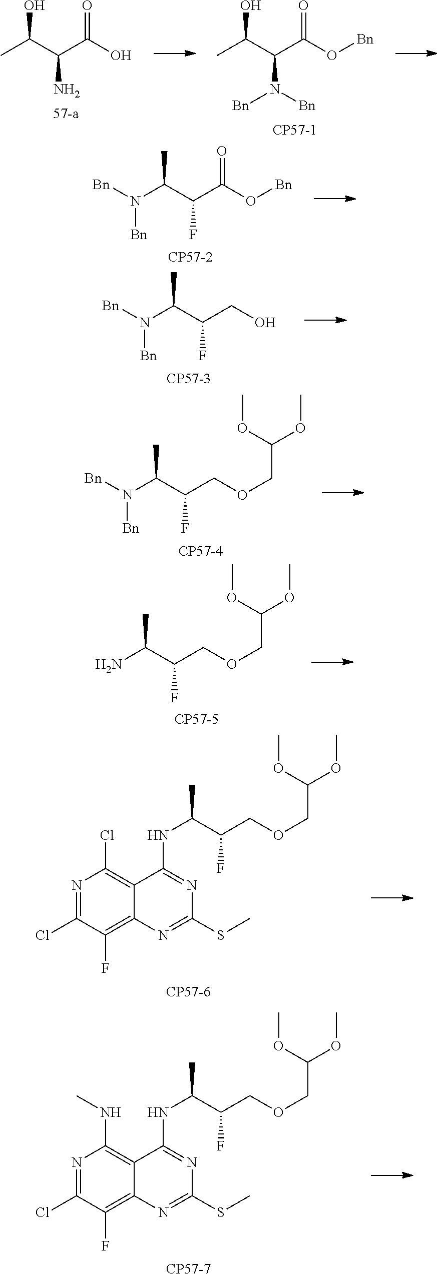

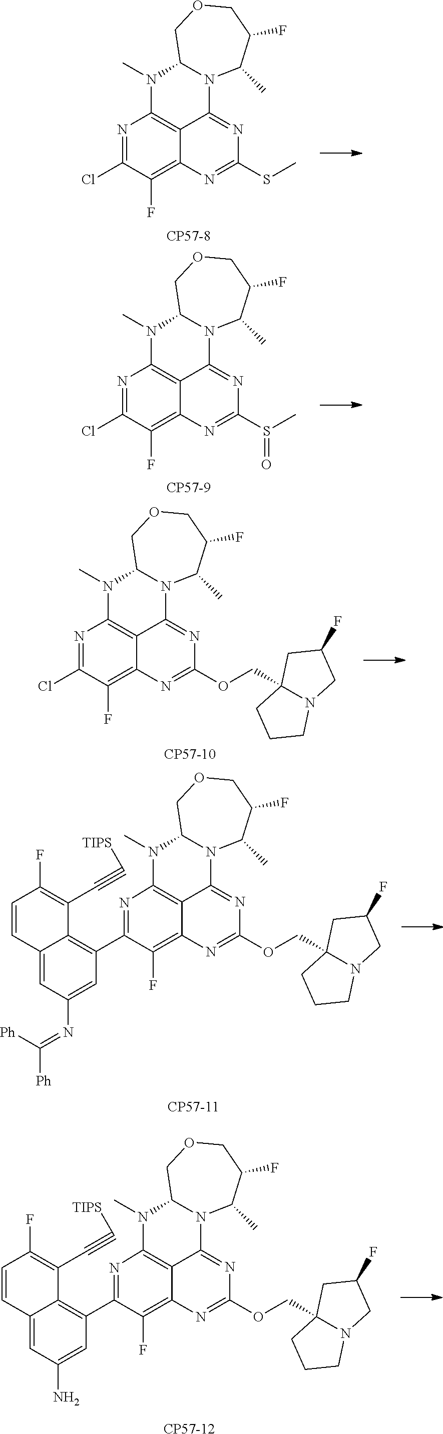

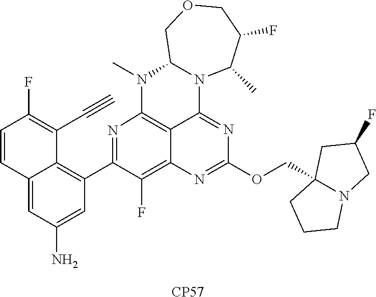

A solution of 57-a (5.09 g), benzyl bromide (22.49 g), K₂CO₃ (12.03 g), NaOH (3.45 g) in water (100 mL) was stirred at 100° C. for 3 h. The solution was extracted with EA and post processed to afford CP57-1 (6641 mg). MS: m/z: 390[M+H]⁺.

To a solution of CP57-1 (1029 mg) in THF (10 mL) was added DAST (508 mg) at RT and the resulting mixture was stirred for 3 h, quenched with 10% Na₂CO₃ solution, extracted with EA and post processed to afford CP57-2 (1046 mg). MS: m/z: 392[M+H]⁺.

A solution of CP57-2 (6.62 g) in THF (60 mL) was stirred at 0° C. The mixture was added LiAlH₄ (1297 mg) at 0° C. and stirred at RT for 2 h, quenched with Na₂SO₄ decahydrate, filtered, concentrated and purified to afford CP57-3 (4513 mg). MS: m/z: 288[M+H]⁺.

A solution of CP57-3 (792 mg) in THF (15 mL) was stirred at 0° C. The mixture was added sodium hydride (414 mg) at 0° C. and stirred at RT for 0.5 h. The mixture was added DMAP (62 mg), tetrabutylammonium iodide (202 mg) and bromoacetaldehyde dimethyl acetal (1014 mg) and stirred at 70° C. for 6 h. The mixture was allowed to cool to RT, quenched with water, extracted with EA and post processed to afford CP57-4 (503 mg).

¹HNMR (400 MHz, CDCl₃) δ7.39 (d, J=7.3 Hz, 4H), 7.33 (t, J=7.5 Hz, 4H), 7.29-7.24 (m, 2H), 4.72-4.52 (m, 1H), 4.46 (t, J=5.2 Hz, 1H), 3.99-3.88 (m, 3H), 3.60-3.42 (m, 5H), 3.39 (d, J=2.3 Hz, 6H), 3.06-2.86 (m, 1H), 1.20 (d, J=6.9 Hz, 3H). MS: m/z: 376[M+H]⁺.

A solution of CP57-4 (200 mg), Pd(OH)₂/C (237 mg) in MeOH (10 mL) was stirred at RT for 16 h under H₂. The solution was filtered and concentrated to afford crude CP57-5 (103 mg). MS: m/z: 196[M+H]⁺.

Compound 57 (CP57, confirmed) was synthesized in a manner similar to CP46 with CP57-5. MS: m/z: 648[M+H]⁺.

### Example 59

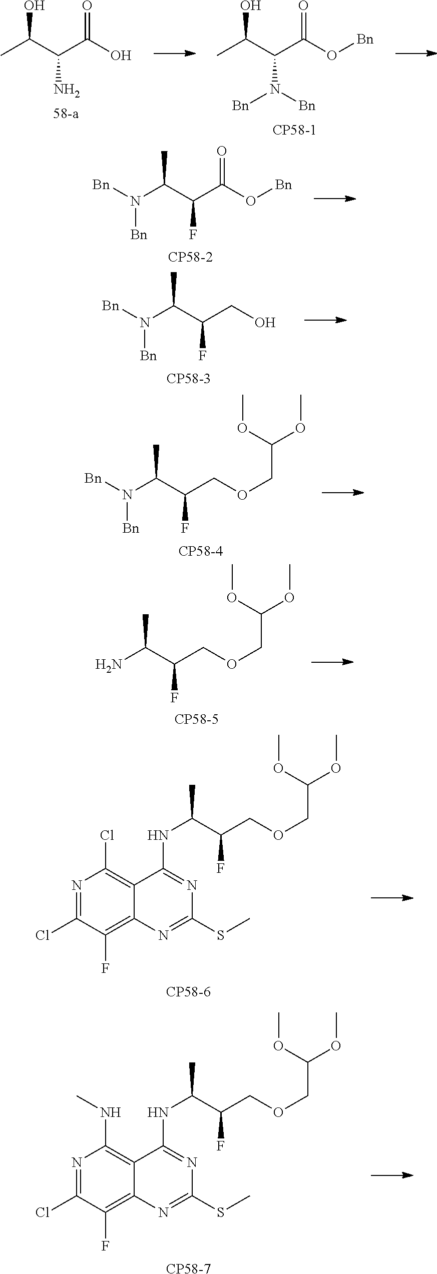

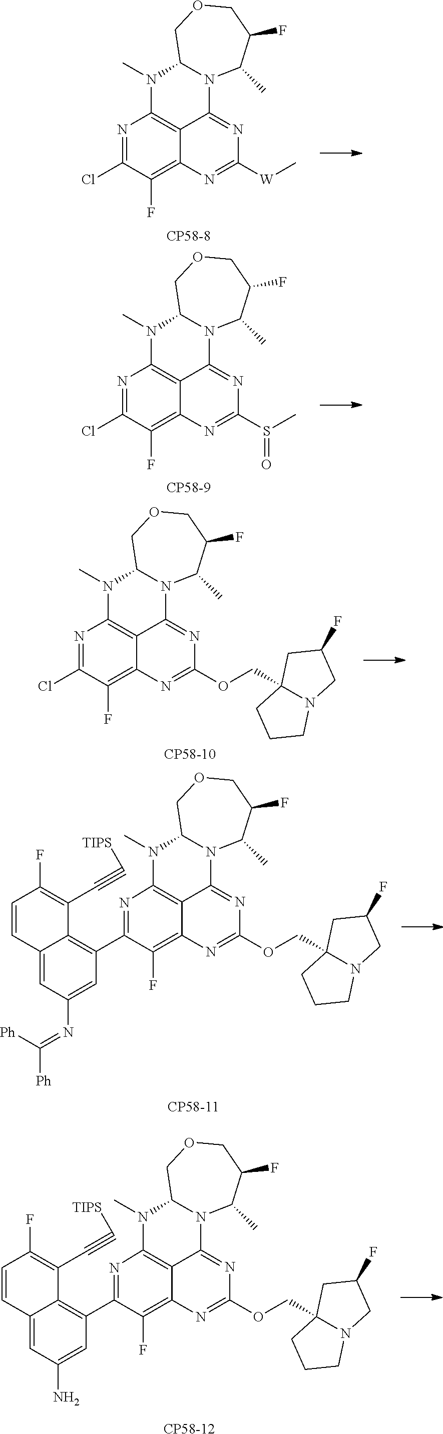

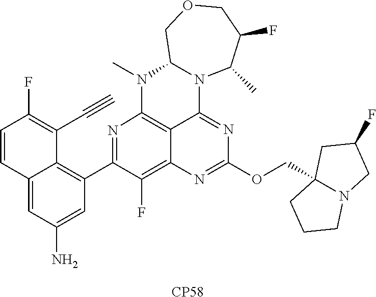

Compound 58 (CP58, confirmed) was synthesized in a manner similar to CP57 with 58-a. MS: m/z: 648[M+H]⁺.

### Example 60

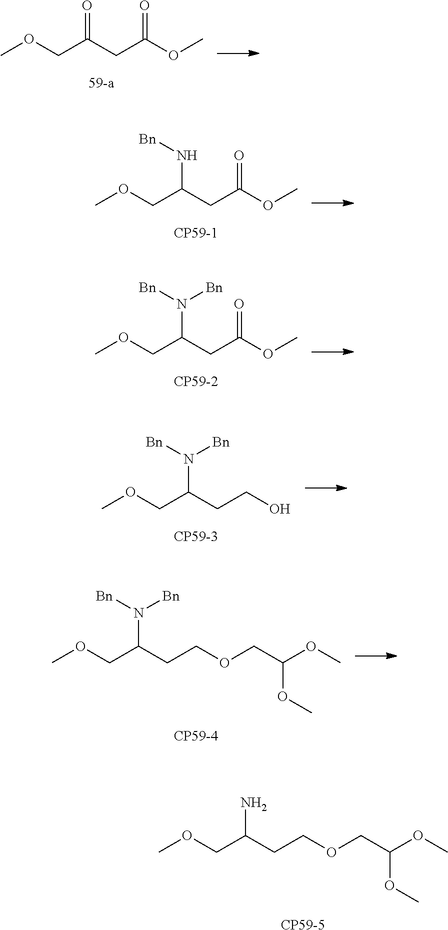

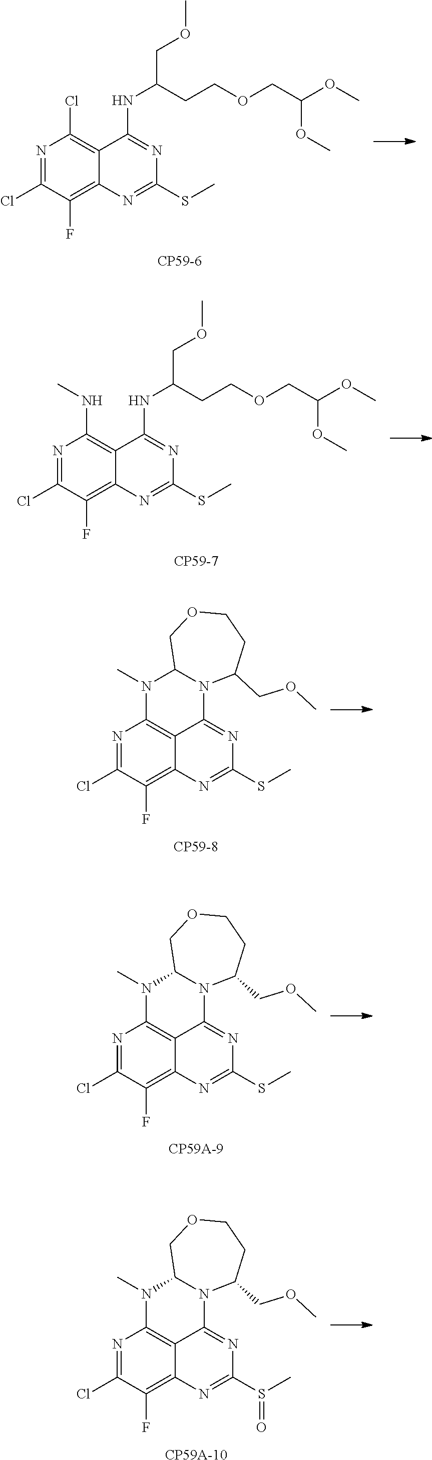

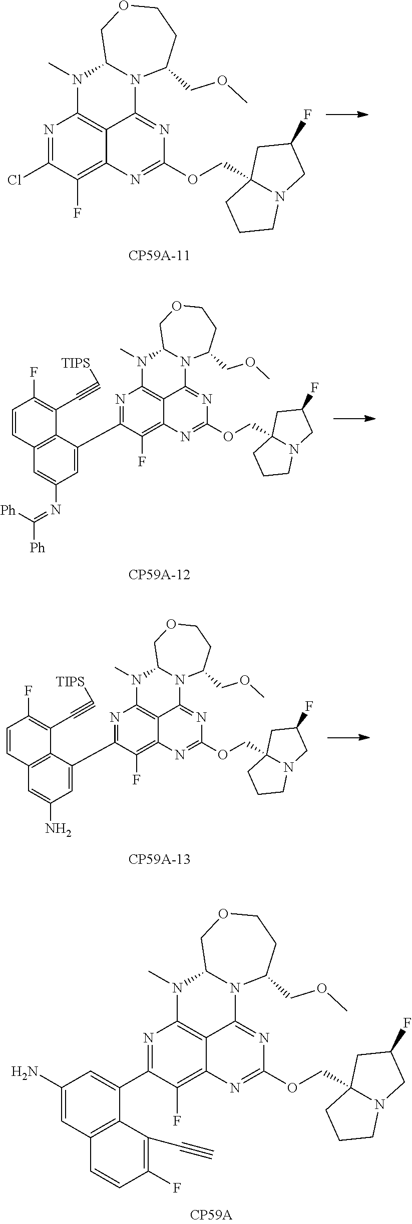

CP59-8 was synthesized in a manner similar to CP94-4 with CP59-5.

The CP59-8 (519 mg) was separated by Prep-HPLC-Gilson with the conditions: Column, CHIRAL ART Cellulose-SA column (2 cm×25 cm, 5 um; mobile phase, (Hex:DcM=3:1)(0.1% DEA)/EtOH (50:50); Flowing rate: 18 ml/min. This resulted in CP59A-9 (173 mg, the first eluting isomer, Retention time: 4.587 min) and CP59B-9 (190 mg, the second eluting isomer, Retention time: 5.067 min).

Compound 59A (CP59A, confirmed) was synthesized in a manner similar to CP46 with CP59A-9. MS: m/z: 660[M+H]⁺.

### Example 61

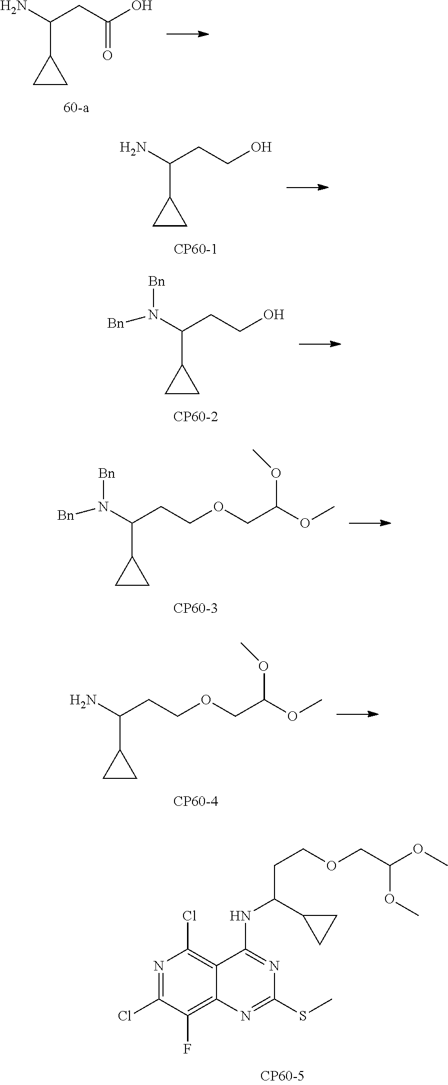 [SMILES: NC(CC(=O)O)C1CC1 ; NC(CCO)C1CC1 ; OCCC(C1CC1)N(Cc1ccccc1)Cc1ccccc1 ; COC(COCCC(C1CC1)N(Cc1ccccc1)Cc1ccccc1)OC ; COC(COCCC(N)C1CC1)OC ; COC(COCCC(Nc1nc(SC)nc2c(F)c(Cl)nc(Cl)c12)C1CC1)OC]

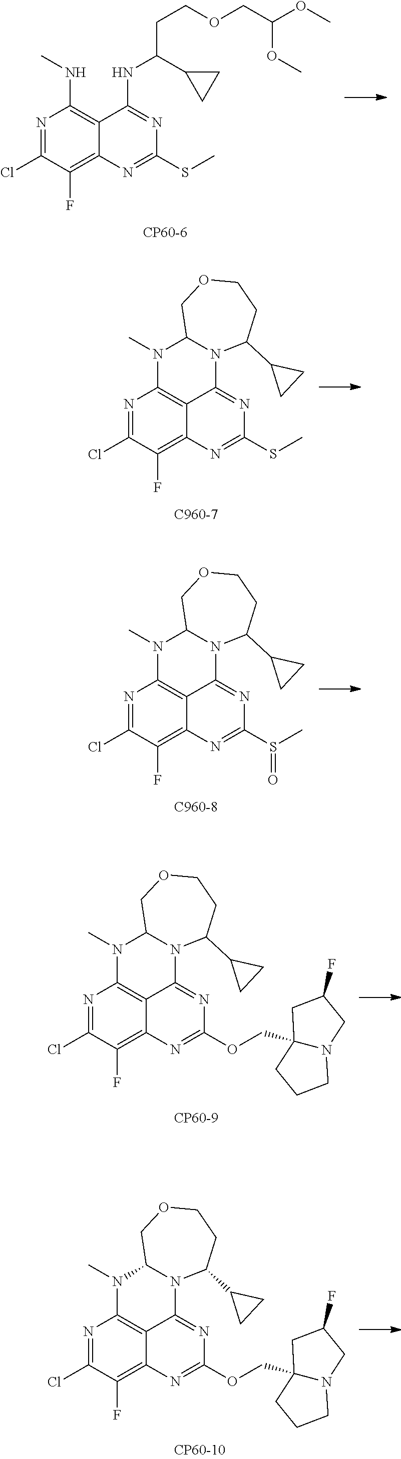 [SMILES: CNc1nc(Cl)c(F)c2nc(SC)nc(NC(CCOCC(OC)OC)C3CC3)c12 ; CSc1nc2c3c(nc(Cl)c(F)c3n1)N(C)C1COCCC(C3CC3)N21 ; CN1c2nc(Cl)c(F)c3nc(S(C)=O)nc(c23)N2C(C3CC3)CCOCC12 ; CN1c2nc(Cl)c(F)c3nc(OC[C@@]45CCCN4C[C@H](F)C5)nc(c23)N2C(C3CC3)CCOCC12 ; CN1c2nc(Cl)c(F)c3nc(OC[C@@]45CCCN4C[C@H](F)C5)nc(c23)N2[C@@H](C3CC3)CCOC[C@@H]12]

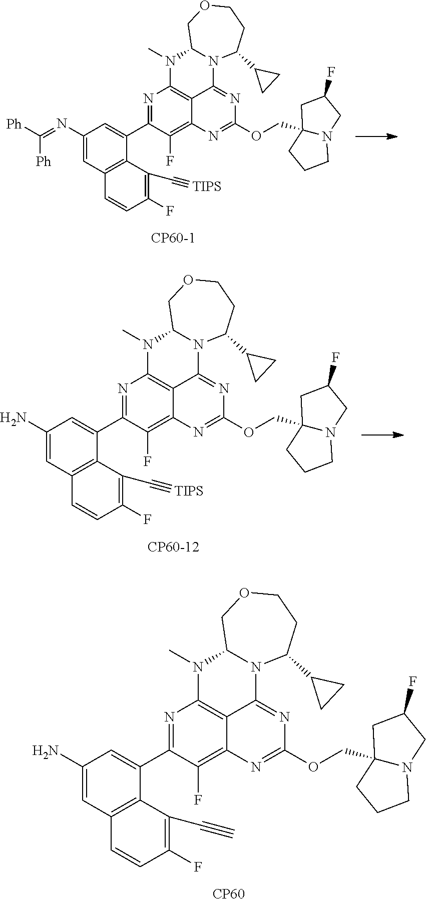

To a solution of 60-a (2.162 g) in THF (20 mL) was added LAH (1222 mg) in batches at −10° C. The reaction mixture was stirred at 40° C. for 0.75 h and quenched with water (1.5 mL), aq. NaOH (1.5 mL, 15% w/w) and water (5 mL). The mixture was filtered and the filtrate concentrated and purified to give CP60-1 (2.852 mg).

CP60-9 was synthesized in a manner similar to CP55-8 with CP60-1.

CP60-9 (331 mg) was separated by Prep-HPLC-Gilson with the conditions: Column, CHIRAL ART Cellulose-SC column (2 cm×25 cm, 5 um); mobile phase, (Hex:DCM=3:1)(0.1% DEA)/EtOH (50:50); Flowing rate: 18 mL/min. This resulted in CP60-10 (138 mg, the second eluting isomer, Retention time: 8.740 min).

Compound 60 (CP60, confirmed) was synthesized in a manner similar to CP46 with CP60-10. MS: m/z: 656 [M+H]⁺.

### Example 62

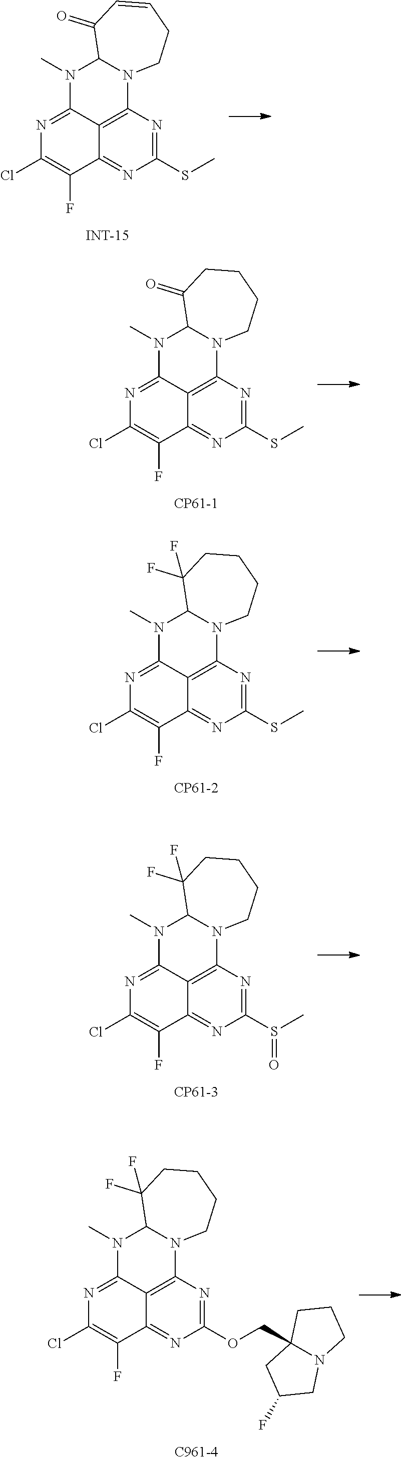

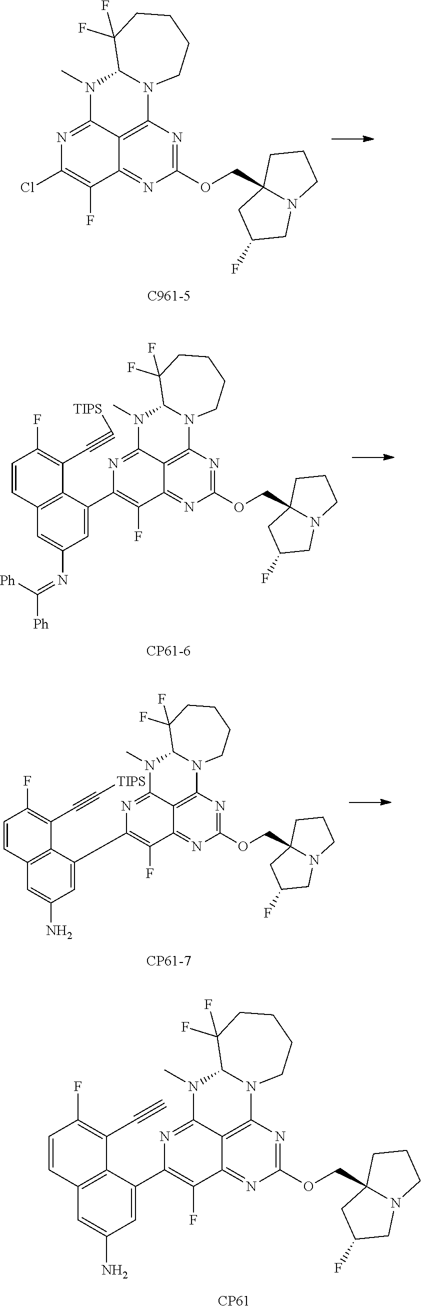

A solution of INT15 (1.005 g) in THF (100 mL) and DCM (100 mL) was added Pd/C (459 mg). The mixture was stirred under H₂ atmosphere at RT for 3 h. The solution was filtered and the filtration was concentrated to afford CP61-1 (1053 mg). MS: m/z: 368 [M+H]⁺.

A solution of CP61-1 (0.552 g) in DCM (50 mL) was added DAST in dropwise (1.603 g) at 0° C. and stirred overnight at RT, quenched with 10% Na₂CO₃ solution, extracted with DCM and post processed to afford CP61-2 (400 mg). MS: m/z: 390 [M+H]⁺.

CP61-4 was synthesized in a manner similar to CP46-10 with CP61-2.

CP61-4 was separated by chiral-HPLC with the conditions: CHIRAL ART Cellulose-SC, 20 mm×250 mm, 5 um; mobile phase, Hex (0.1% DEA)/EtOH (80:20); Flowing rate: 15 ml/min to afford CP61-5 (the second eluting isomer, retention time 9.189 min). MS: m/z: 501 [M+H]⁺.

Compound 61 (CP61, confirmed) was synthesized in a manner similar to CP46 with CP61-5. MS: m/z: 650 [M+H]⁺.

### Example 63

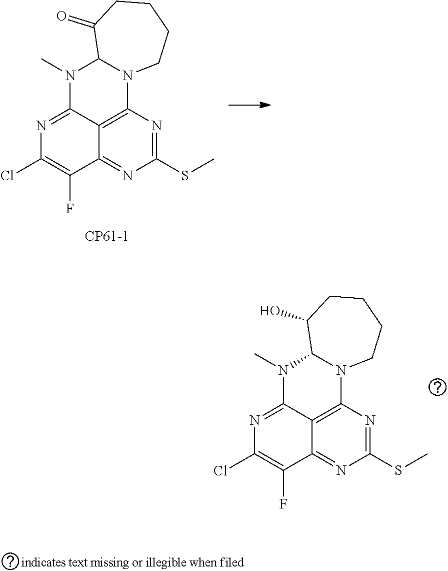 [SMILES: "CP61-1" ; CSc1nc2c3c(nc(Cl)c(F)c3n1)N(C)C1C(=O)CCCCN21 ; CSc1nc2c3c(nc(Cl)c(F)c3n1)N(C)[C@@H]1[C@H](O)CCCCN21]

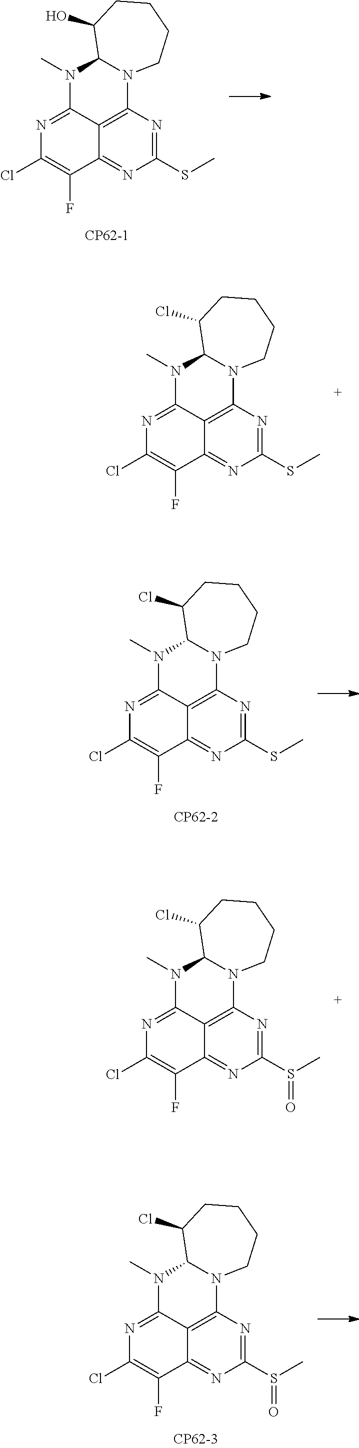

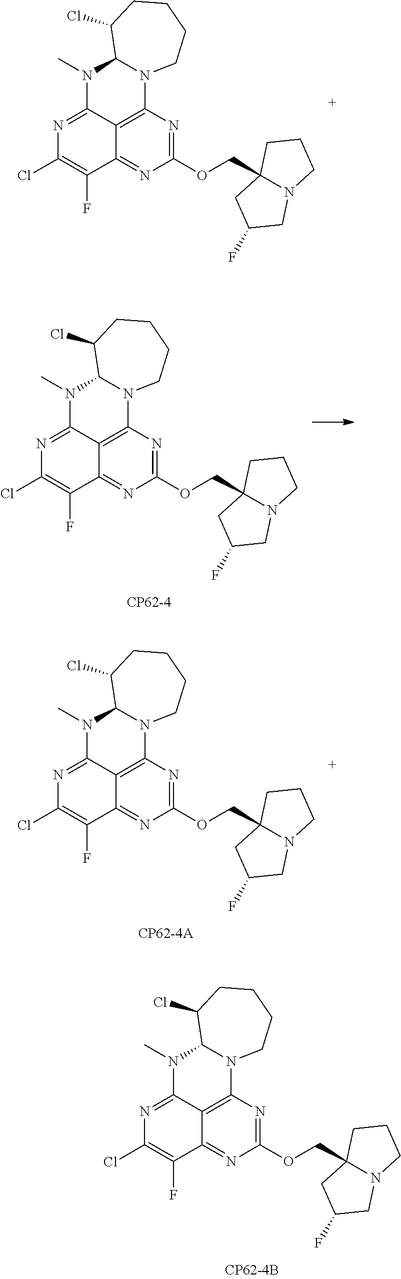 [SMILES: CN1c2nc(Cl)c(F)c3nc(OC[C@@]45CCCN4C[C@H](F)C5)nc(c23)N2CCCC[C@@H](Cl)[C@H]12 ; CN1c2nc(Cl)c(F)c3nc(OC[C@@]45CCCN4C[C@H](F)C5)nc(c23)N2CCCC[C@H](Cl)[C@@H]12 ; CN1c2nc(Cl)c(F)c3nc(OC[C@@]45CCCN4C[C@H](F)C5)nc(c23)N2CCCC[C@@H](Cl)[C@H]12 ; CN1c2nc(Cl)c(F)c3nc(OC[C@@]45CCCN4C[C@H](F)C5)nc(c23)N2CCCC[C@H](Cl)[C@@H]12]

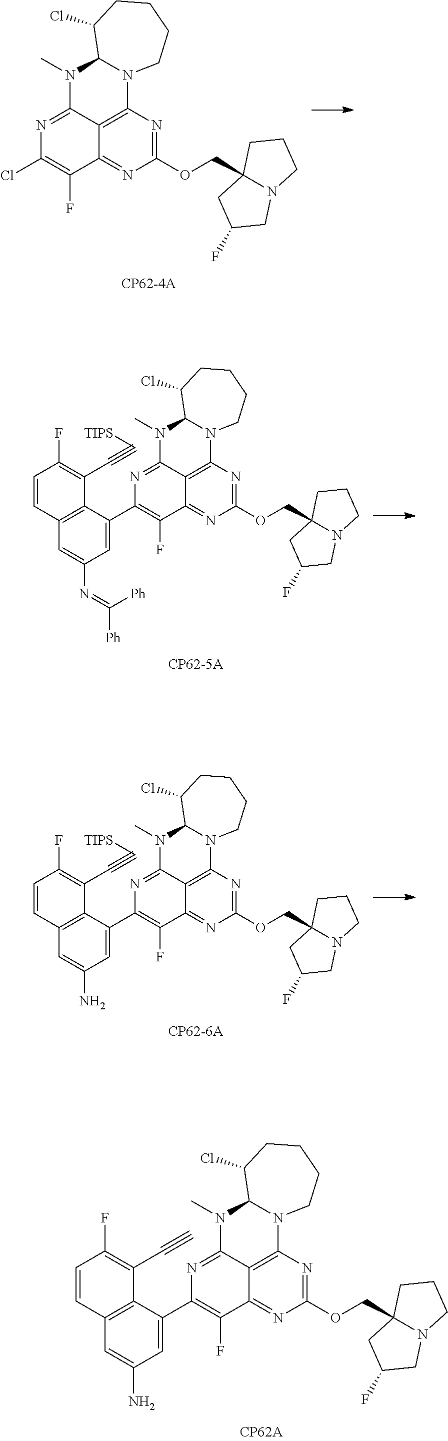

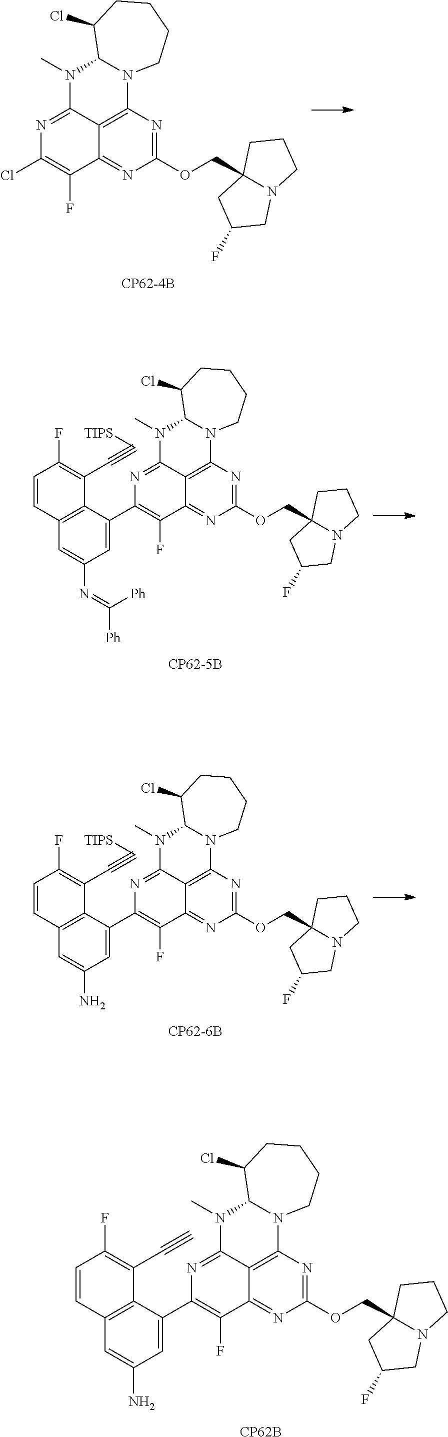

To a solution of CP61-1 (0.800 g) in DCM (20 mL) and THF (20 mL) was added NaBH₄ (255 mg) and the resulting mixture was stirred 4 h at RT, quenched with water, extracted with DCM and post processed to afford CP62-1 (735 mg). MS: m/z: 370[M+H]⁺.

To a solution of triphenylphosphine (1085 mg) in DCM (25 mL) was added CCl₄ (1008 mg) at 0° C. under N₂ atmosphere and the resulting mixture was stirred 20 min at 0° C. A solution of CP62-1 (0.636 g) in DCM (5 mL) was dropwised at 0° C., stirred overnight at RT, diluted with DCM (30 mL), washed with H₂O and saturated NaCl (aq) and post processed to give CP62-2 (338 mg). MS: m/z: 388[M+H]⁺.

CP62-4 was synthesized in a manner similar to CP46-10 with CP62-2.

CP62-4 was separated by Prep-HPLC-Gilson with the conditions: Column, CHIRAL ART Cellulose-SC column (2 cm×25 cm, 5 um), mobile phase, (Hex:DCM=3:1)(0.1% DEA)/IPA (50:50); Flowing rate: 15 ml/min. This resulted in CP62-4A (76 mg, the first eluting isomer, Retention time 6.223 min) and CP62-4B (66 mg, the second eluting isomer. Retention time 7.320 min). MS: m/z: 499[M+H]⁺.

Compound 62B (CP62B, confirmed) was synthesized in a manner similar to CP46 with CP62-4B. MS: m/z: 648[M+H]⁺.

Compound 62A (CP62A, confirmed) was synthesized in a manner similar to CP46 with CP62-4A. MS: m/z: 648[M+H]⁺.

### Example 64

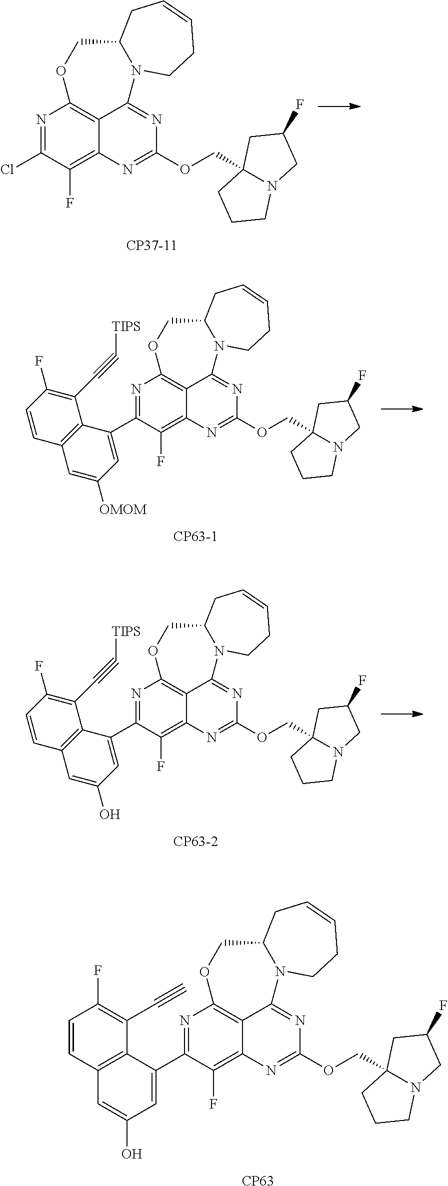

Compound 63 (CP63, confirmed) was synthesized in a manner similar to CP2 with CP37-11. MS (ESI, m/z): 614[M+H]⁺.

### Example 65

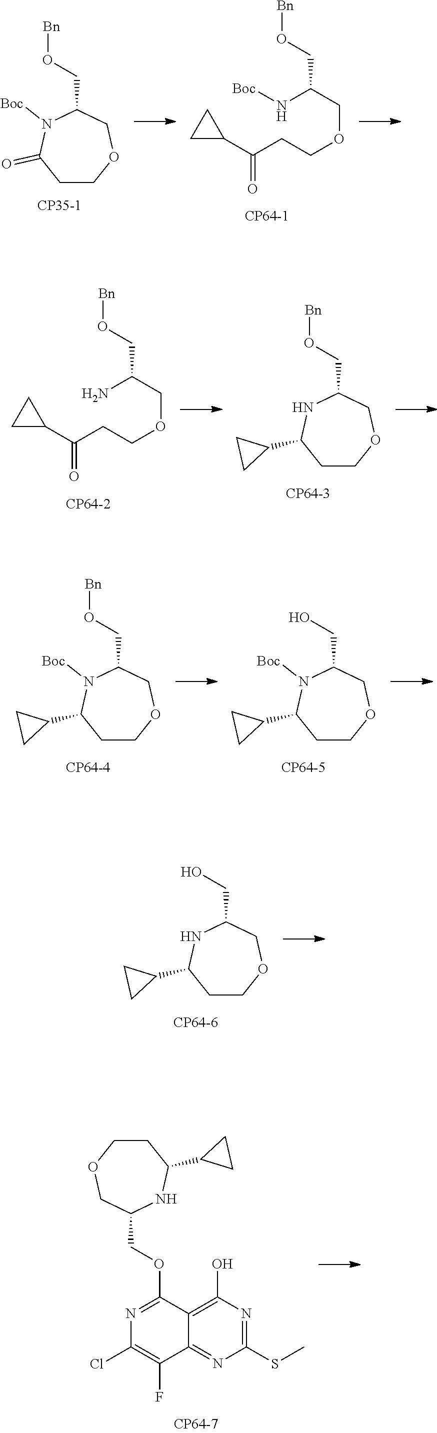

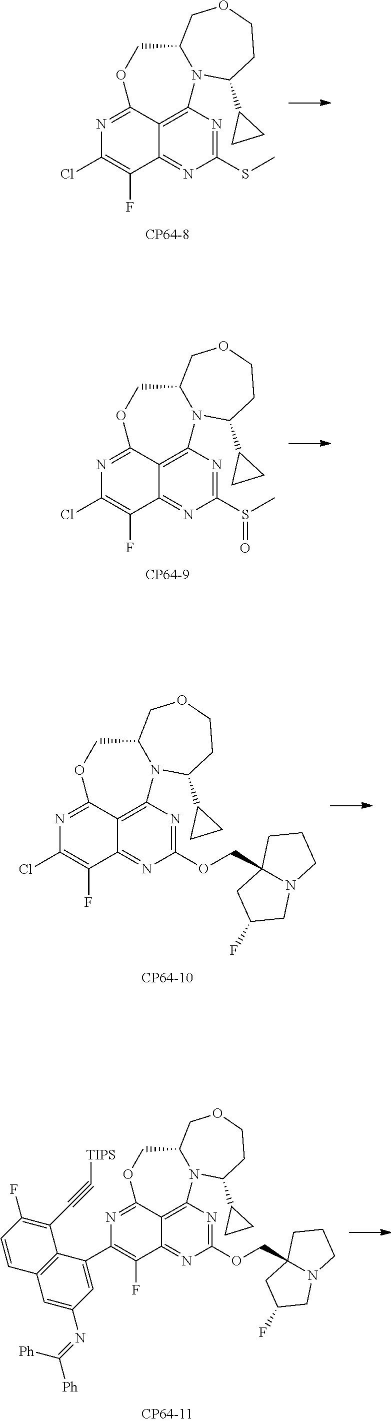

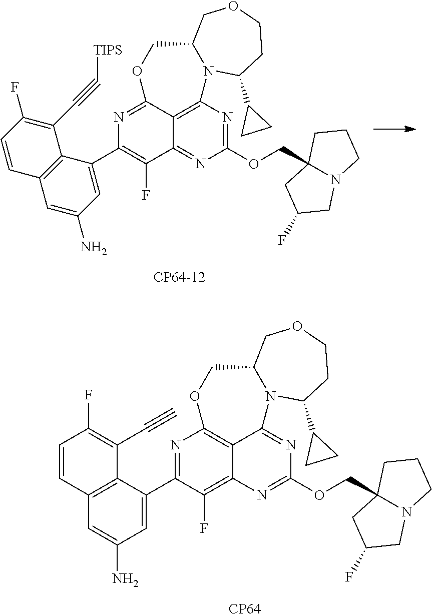

To a solution of CP35-1 (2.03 g) in THF (20 mL) was added cyclopropylmagnesium bromide (12 mL, 1M in THF) dropwise at −78° C. The reaction mixture was stirred at −78° C. for 1.5 hour under N₂, quenched with sat. aq. NH₄Cl, diluted with EA and water and post processed to give CP64-1 (1.80 g). MS: m/z: 378[M+H]⁺.

To a solution of CP64-1 (1.80 g) in DCM (20 mL) was added TFA (5 mL) at RT. The reaction mixture was stirred at RT overnight and then concentrated to give crude CP64-2 (1.32 g). MS: m/z: 278[M+H]⁺.

To a solution of CP64-2 (1.32 g) in DCE (20 mL) were added sodium triacetoxyborohydride (3.75 g) and anhydrous Na₂SO₄ (5.73 g). The mixture was stirred at 42° C. for two days. The pH value of the mixture was adjusted to 9 by aq. K₂CO₃, then extracted by DCM and post processed to give crude CP64-3 (1.35 g). MS: m/z: 262[M+H]⁺.

To a solution of CP64-3 (1.35 g) in THF (15 mL) was added di-tert-butyl dicarbonate (1.57 g). The reaction mixture was stirred at 44° C. overnight under N₂, diluted with EA (50 mL) and water (30 mL) and post processed to give CP64-4 (683 mg). MS: m/z: 362[M+H]⁺.

To a solution of CP64-4 (683 mg) in MeOH (20 mL) was added Pd(OH)₂/C (0.31 g). The reaction mixture was stirred at 44° C. for 24 hours under H₂, filtered and the filtrate was concentrated to give CP64-5 (0.47 g). MS: m/z: 272[M+H]⁺.

Compound 64 (CP64, confirmed) was synthesized in a manner similar to CP16 with CP64-5. MS: m/z: 657[M+H]⁺.

### Example 66

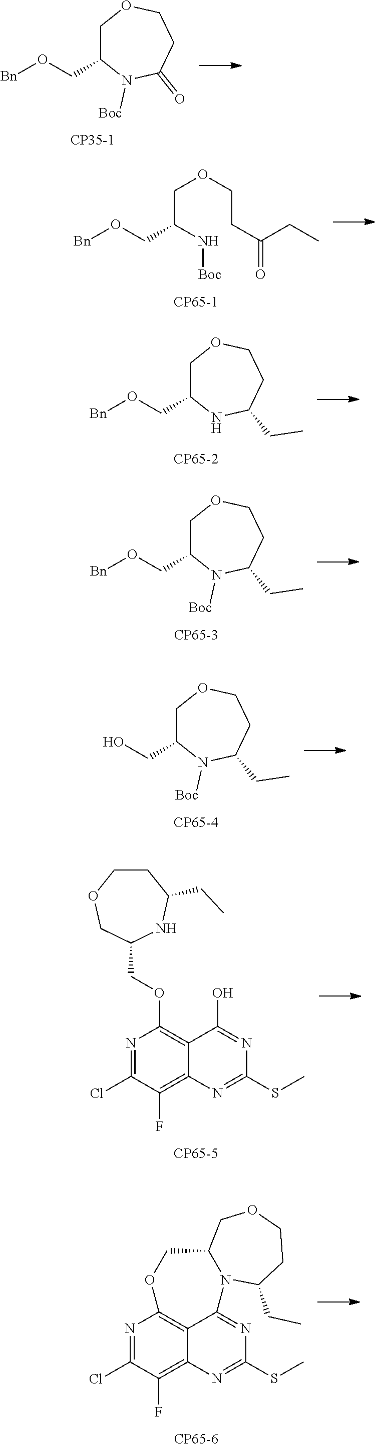

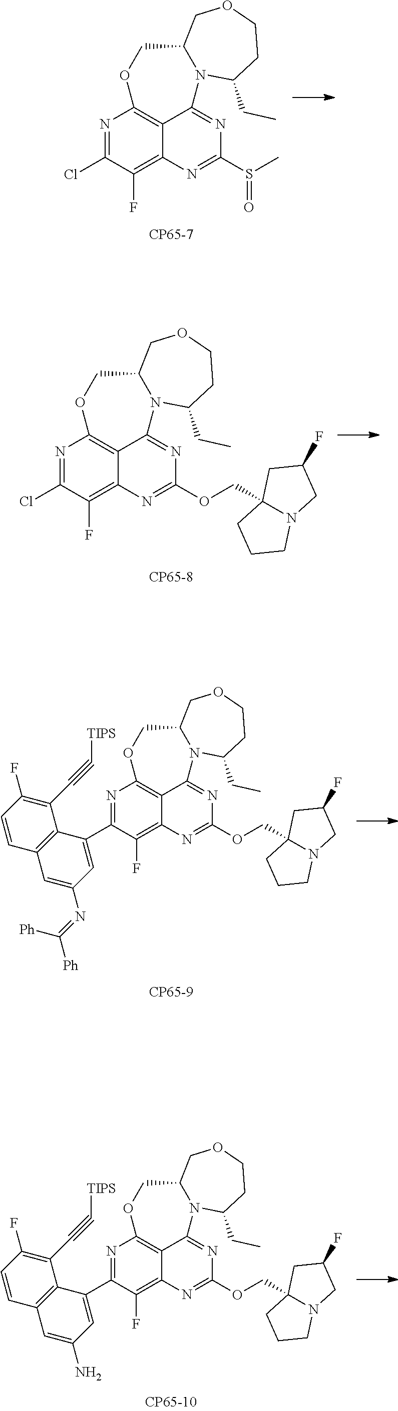

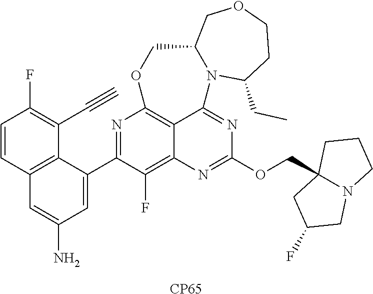

Compound 65 (CP65, confirmed) was synthesized in a manner similar to CP64. MS: m/z: 645[M+H]⁺.

### Example 67

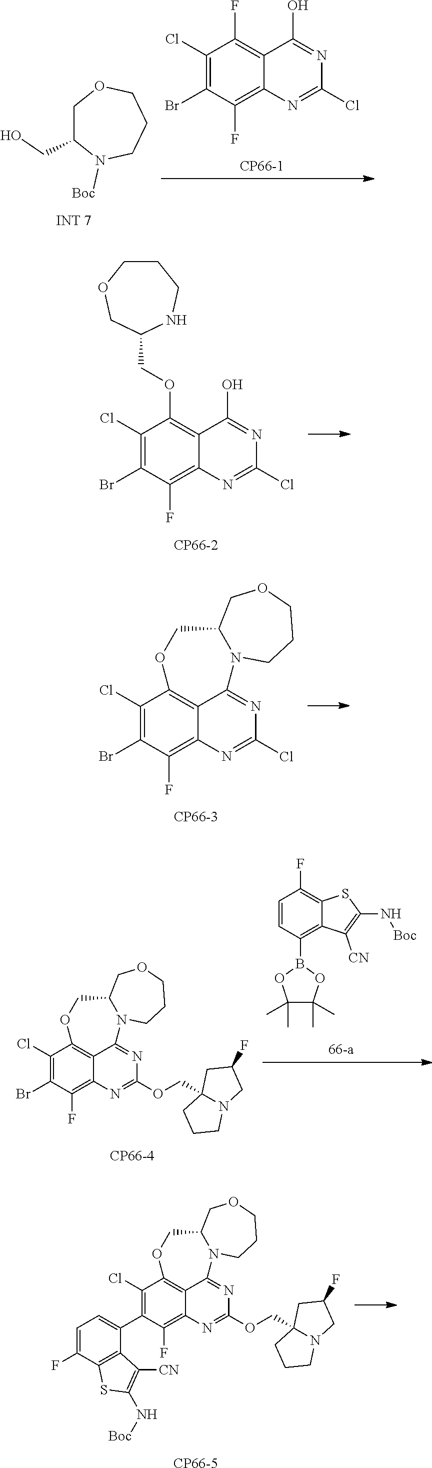

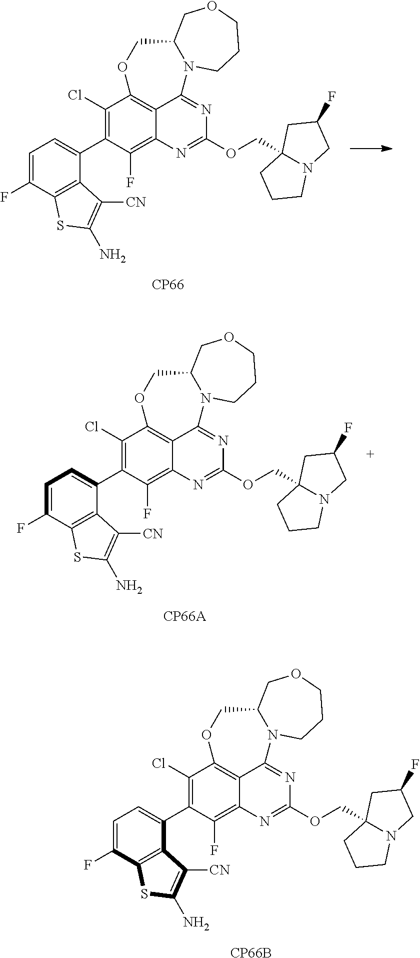

To a solution of INT7 (0.63 g) in MeOH (10 mL) was added HCl (5 mL, 4M in 1,4-dioxane). The reaction mixture was stirred at RT for 2.5 hours and concentrated to give the residue. To a mixture of the residue in THF (15 mL) was added NaH (550 mg, 60% content in oil) in batches and CP66-1 (899 mg) at −10° C. The reaction mixture was stirred for 20 h at RT, quenched with a little of water and concentrated to give the residue. To the residue was added DCM:MeOH=10:1 (20 mL). After stirring for 5 min, the mixture was filtered and the filtrate concentrated to afford CP66-2 (1483 mg). MS (ESI, m/z): 442 [M+H]⁺.

To a solution of CP66-2 (1483 mg) in DCM (20 mL) was added DIEA (1.51 g) and BOP—Cl (1.68 g). The reaction mixture was stirred at RT for 21 hours, quenched with water and extracted with DCM. The organic layer was washed with brine, dried, concentrated and purified by Prep-TLC to afford CP66-3 (190 mg). MS (ESI, m/z): 424 [M+H]⁺.

To a solution of CP66-3 (190 mg) in DMF (1 mL) and THF (1 mL) was added INT14 (127 mg), DABCO (18 mg) and Cs₂CO₃ (216 mg) at 30° C. and the resulting mixture was stirred for 20 h, quenched with water and extracted with EA. The organic layer was washed with brine, dried, concentrated and purified by Prep-TLC to afford CP66-4 (206 mg). MS (ESI, m/z): 547 [M+H]⁺.

A mixture of CP66-4 (116 mg), 66-a (98 mg), DPEPhosPdCl₂ (22 mg) and Cs₂CO₃ (213 mg) in toluene (3 mL) was stirred at 105° C. for 19 hours under N₂. The reaction was added water and extracted with EA. The organic layer was washed with brine, dried and concentrated. The residue was purified by Prep-TLC to afford CP66-5 (148 mg). MS (ESI, m/z): 757 [M+H]⁺.

To a solution of CP66-5 (148 mg) in DCM (6 mL) was added TFA (2 mL). The reaction mixture was stirred at RT for 2 hours and concentrated. The residue was purified by Prep-HPLC (Agela Durashell C18, 30 mm×250 mm, 10 μm, phase A: 0.1% TFA in water, phase B: CH₃CN, Gradient: 15% B to 40% B in 37 min at a flow rate of 60 mL/min, 295 nm) to freeze-dried to afford the title CP66 (60 mg, TFA salt). MS (ESI, m/z): 657 [M+H]⁺.

The CP66 (60 mg, TFA salt) was separated by Prep-HPLC-Gilson with the conditions: Column, CHIRAL ART Amylose-SA column (2 cm×25 cm, 5 μm); mobile phase, (Hex:DCM=3:1) (0.1% DEA)/EtOH (50:50); Flowing rate: 20 ml/min. This resulted in Compound 66A (CP66A, 22.3 mg, the first eluting isomer, Retention time: 3.990 min, confirmed) and Compound 66B (CP66B, 15.1 mg, the second eluting isomer, Retention time: 5.103 min, confirmed).

### Example 68

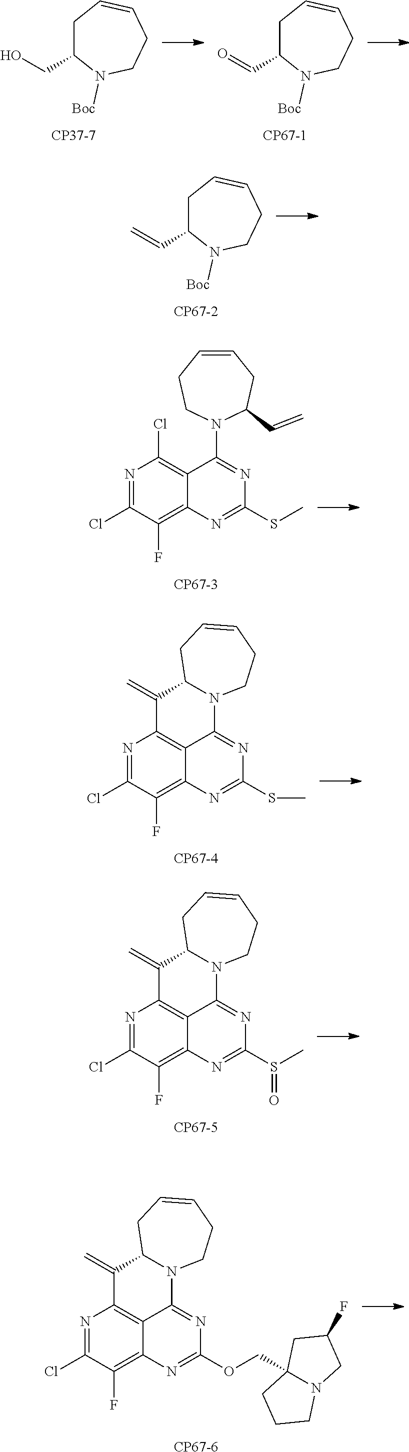

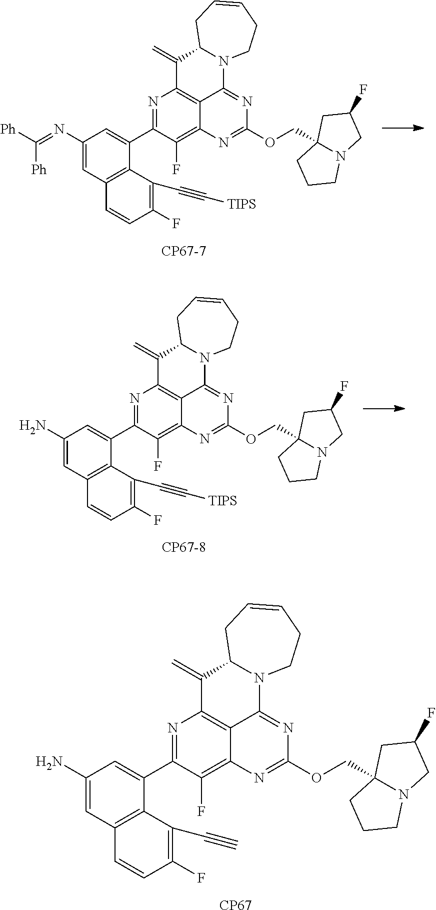

Compound 67 (CP67, confirmed) was synthesized in a manner similar to CP21 with CP37-7. MS: m/z: 609 [M+H]⁺.

### Example 69

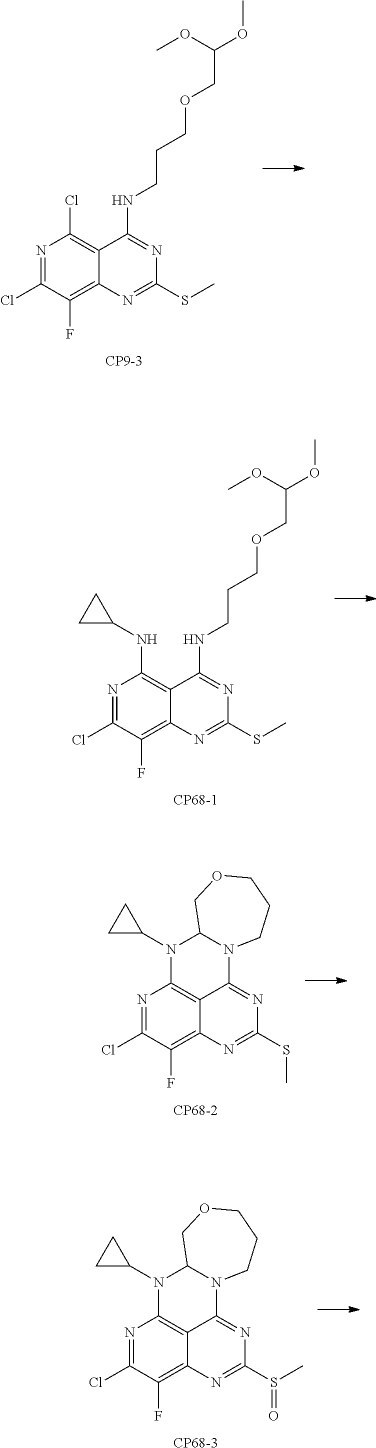

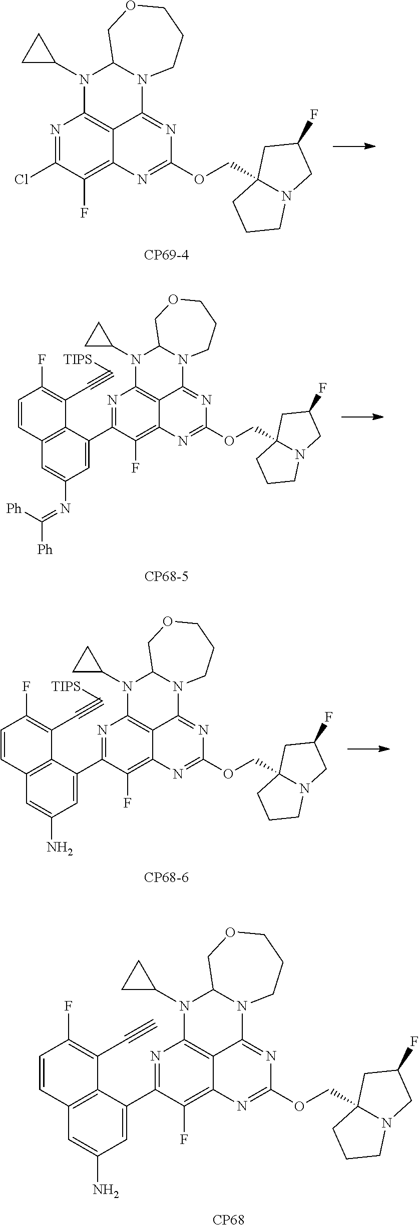

Compound 68 (CP69, confirmed) was synthesized the procedure of CP9. LCMS: m/z: 642[M+H]⁺.

### Example 70

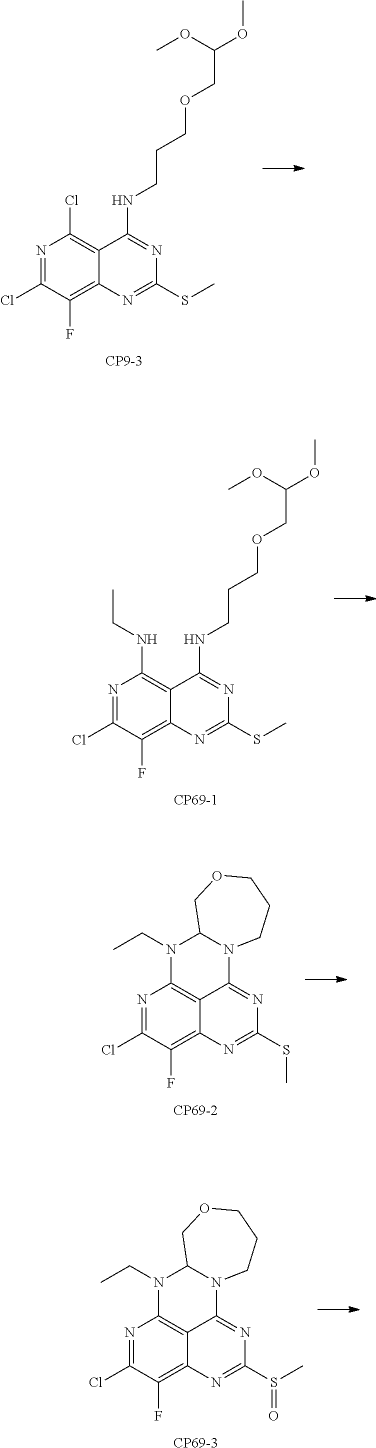

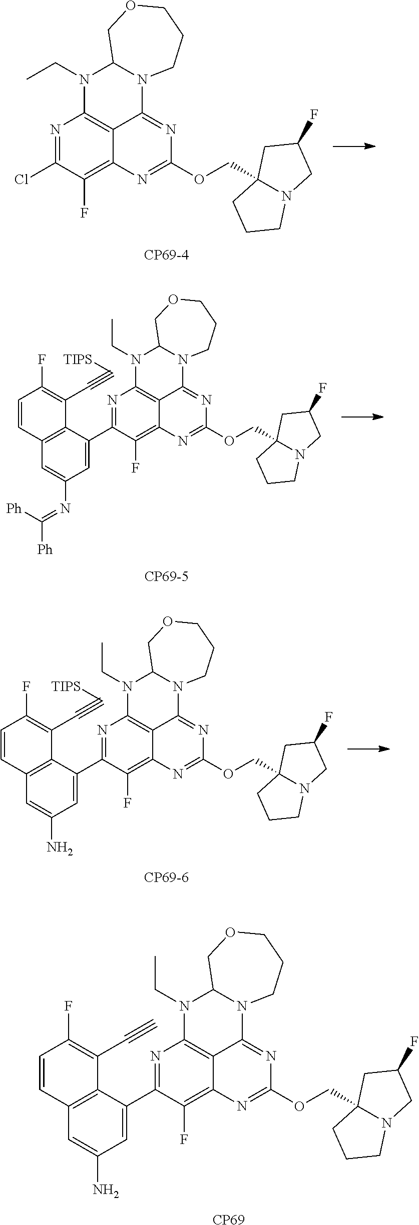

Compound 69 (CP69, TFA salt, confirmed) was synthesized the procedure of CP68. LCMS: m/z: 636[M+H]⁺.

### Example 71

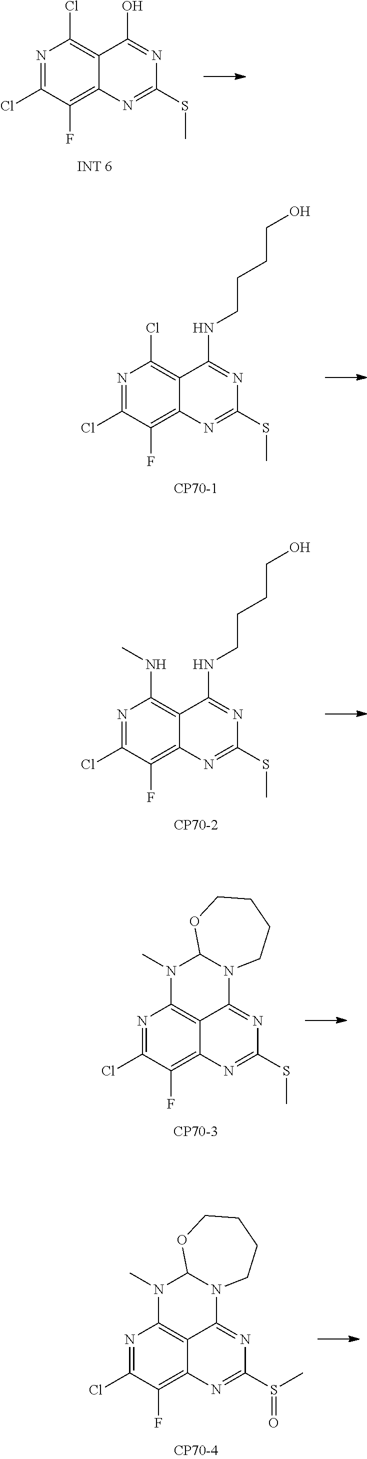 [SMILES: CSc1nc(O)c2c(Cl)nc(Cl)c(F)c2n1 ; CSc1nc(NCCCCO)c2c(Cl)nc(Cl)c(F)c2n1 ; CNc1nc(Cl)c(F)c2nc(SC)nc(NCCCCO)c12 ; CSc1nc2c3c(nc(Cl)c(F)c3n1)N(C)C1OCCCCN21 ; CN1c2nc(Cl)c(F)c3nc(S(C)=O)nc(c23)N2CCCCOC12]

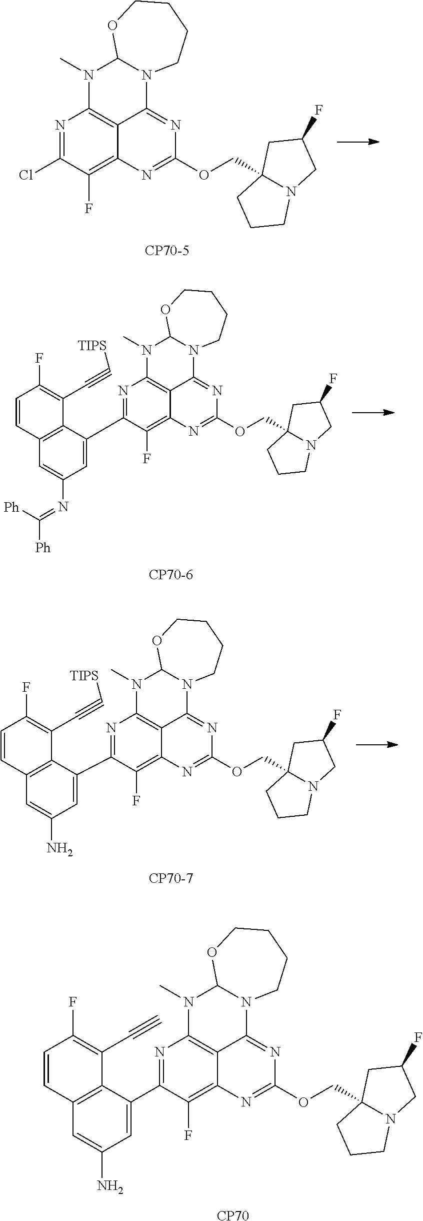

CP70-2 was synthesized the procedure of CP42-6 with 4-aminobutan-1-ol and INT6.

To a solution of CP70-2 (97 mg) in trimethoxymethane (3 mL) was added p-TsOH·H₂O (62 mg) and the mixture was purged with nitrogen followed by stirring at 100° C. for 0.5 hour. The reaction mixture was cooled to RT, diluted with EA and water. The separated organic layer was concentrated and purified to give CP70-3 (157 mg). MS: m/z: 356[M+H]⁺.

Compound 70 (CP70, TFA salt, confirmed) was synthesized the procedure of CP42 with CP70-3. LCMS: m/z: 616[M+H]⁺.

### Example 72

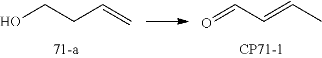

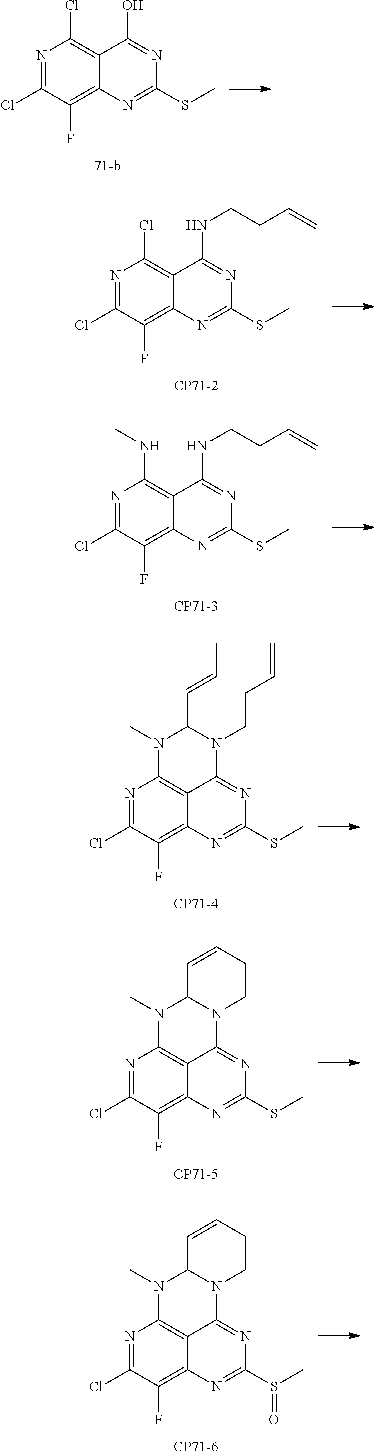 [SMILES: CSc1nc(O)c2c(Cl)nc(Cl)c(F)c2n1 ; C=CCCNc1nc(SC)nc2c(F)c(Cl)nc(Cl)c12 ; C=CCCNc1nc(SC)nc2c(F)c(Cl)nc(NC)c12 ; C=CCCN1c2nc(SC)nc3c(F)c(Cl)nc(c23)N(C)C1/C=C/C ; CSc1nc2c3c(nc(Cl)c(F)c3n1)N(C)C1C=CCCN21 ; CN1c2nc(Cl)c(F)c3nc(S(C)=O)nc(c23)N2CCC=CC12]

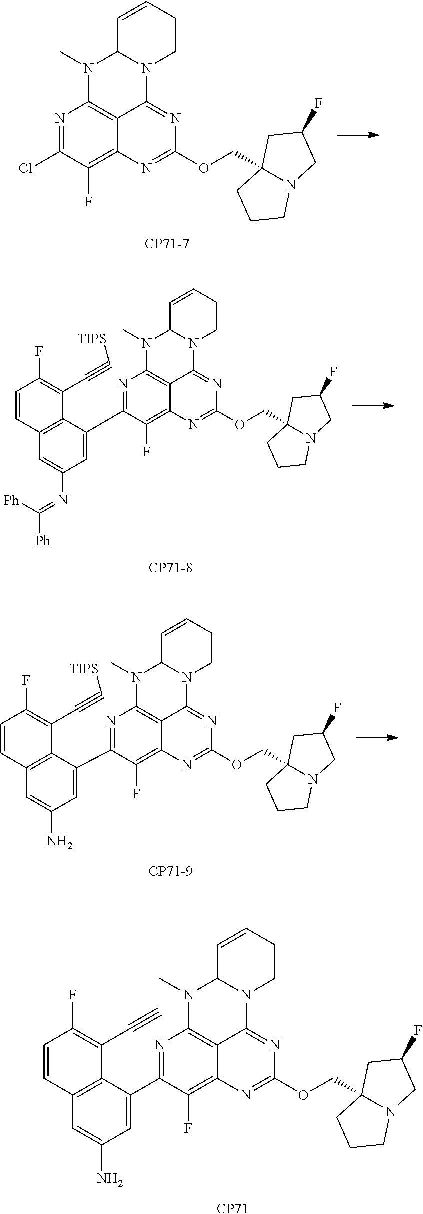

To a solution of 71-a (1.03 g) and Et₃N (7.76 g) in DCM (10 mL) and DMSO (20 mL) was added sulfur trioxide pyridine complex (7.36 g) at 0° C. The resulting mixture was stirred for 4 hours, diluted with DCM and washed with sodium thiosulfate solution (10%), 5% citric acid solution and saturated sodium chloride solution (80 mL). The resulting solution was dried, filtered and concentrated to give CP71-1 (1.06 g).

CP71-5 was synthesized in a manner similar to INT15 with CP71-1.

Compound 71 (CP71, confirmed) was synthesized in the manner similar to CP46 with CP71-5. LCMS: m/z: 598[M+H]⁺.

### Example 73

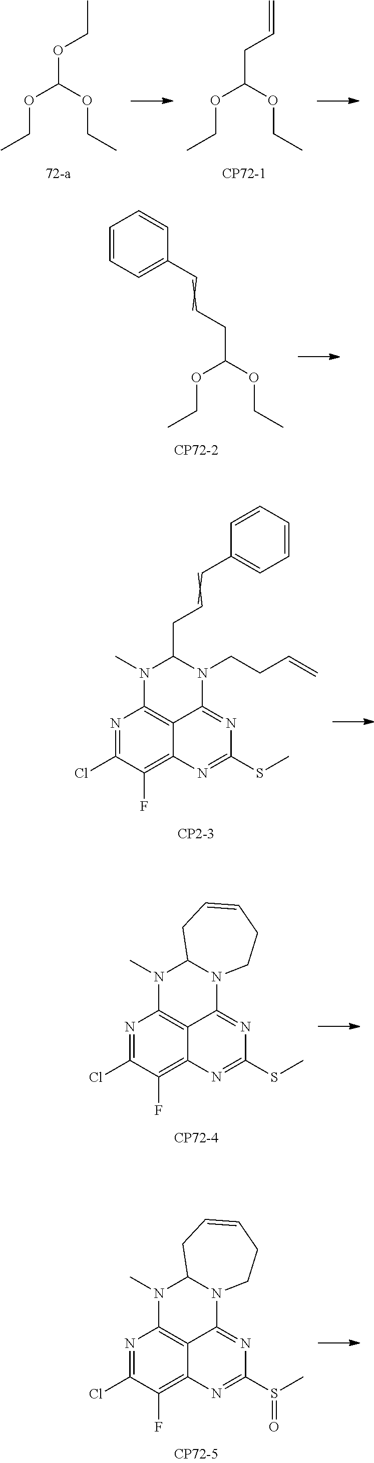

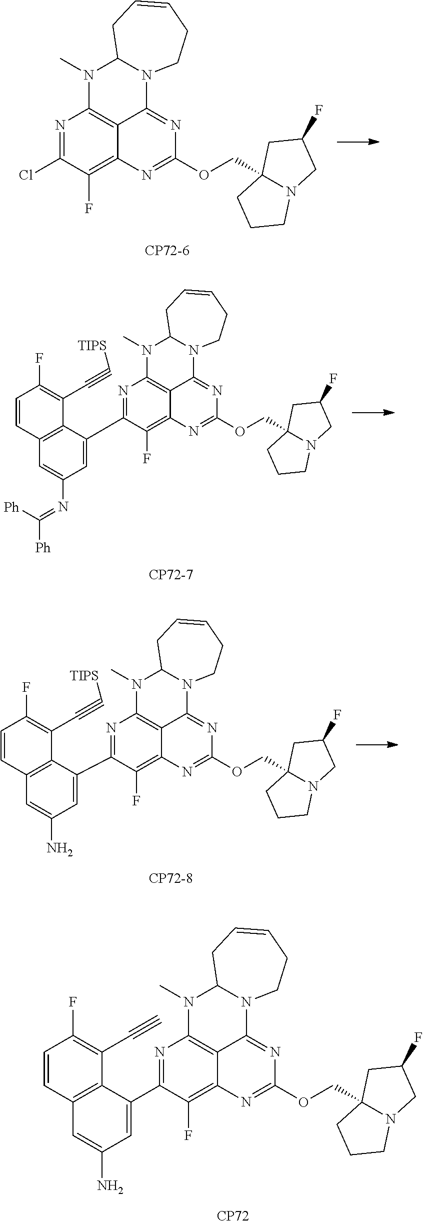

To a solution of allylmagnesium chloride (2M in THF, 15 mL) was added 72-a (1.36 g) under nitrogen at RT and stirred at 80° C. for 20 hours, quenched with saturated ammonium chloride solution (60 mL) and diethyl ether (80 mL). The organic layer was separated, washed with acetic acid solution (1%), saturated NaHCO₃ and saturated sodium chloride solution. The resulting solution was dried, filtered and concentrated to give CP72-1 (2.20 g).

To a solution of CP72-1 (0.85 g), styrene (2.50 g) and titanium ethoxide (1.14 g) in DCE (20 mL) was added Grubbs catalyst 2nd gen (0.39 g). The mixture was purged with nitrogen followed by stirring at 70° C. for 16 hours. Upon completion, the mixture was concentrated and purified to give CP72-2 (0.61 g).

To a solution of CP72-2 (0.543 g), CP71-3 (0.282 g) in DMSO (10 mL) was added p-TsOH·H₂O (160 mg). The mixture was purged with nitrogen followed by stirring at 80° C. for 16 hours. The reaction mixture was cooled to RT, diluted with EA and water. The separated organic layer was concentrated and purified to give CP72-3 (197 mg). MS: m/z: 456[M+H]⁺.

Compound 72 (CP72, confirmed) was synthesized the procedure of CP71 with CP72-3. LCMS: m/z: 612[M+H]⁺.

### Example 74

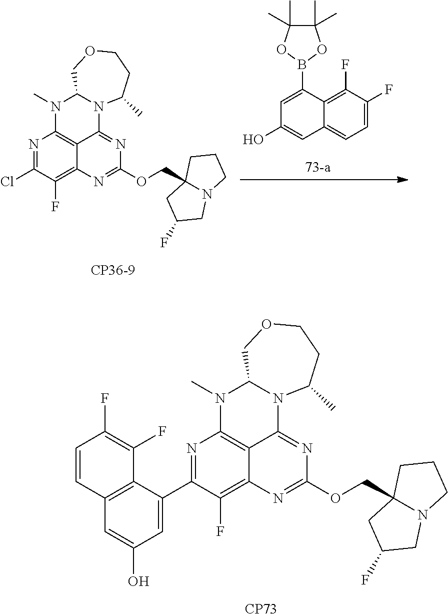 [SMILES: C[C@H]1CCOC[C@H]2N(C)c3nc(Cl)c(F)c4nc(OC[C@@]56CCCN5C[C@H](F)C6)nc(c34)N12 ; CC1(C)OB(c2cc(O)cc3ccc(F)c(F)c23)OC1(C)C ; "CP73" ; C[C@H]1CCOC[C@H]2N(C)c3nc(-c4cc(O)cc5ccc(F)c(F)c45)c(F)c4nc(OC[C@@]56CCCN5C[C@H](F)C6)nc(c34)N12]

To a solution of CP36-9 (228 mg), 73-a (596 mg) and Cs₂CO₃ (482 mg) in toluene (10 mL) and water (2.5 mL) was added cataCXium A Pd G₃ (82 mg) and the mixture was purged with nitrogen followed by stirring at 100° C. for 16 hours. Upon completion, the mixture was diluted with EA and water. The separated organic layer was concentrated and purified to afford Compound 73 (CP73, confirmed). MS: m/z: 625[M+1]⁺.

### Example 75

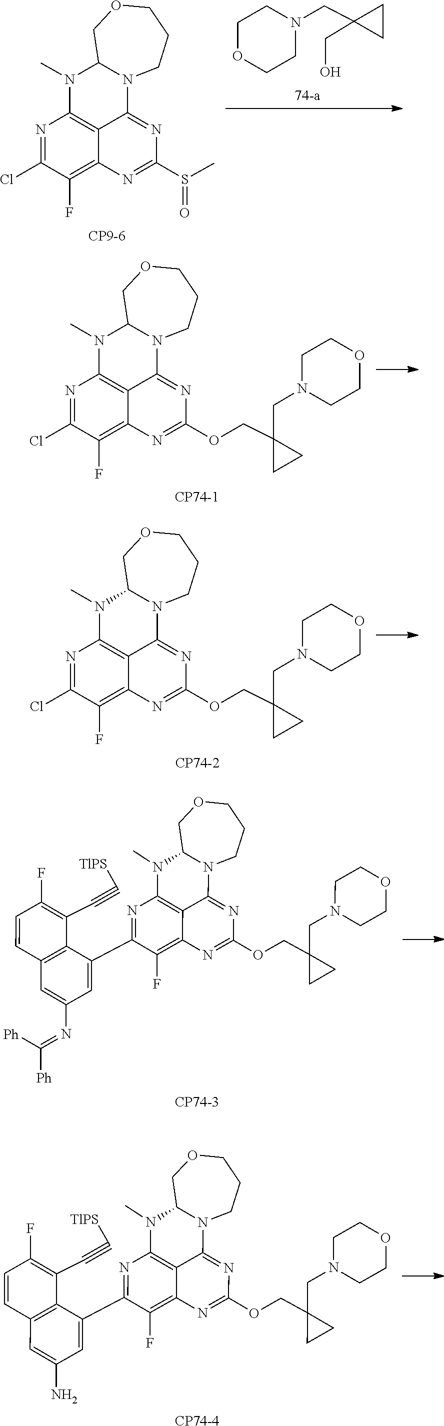

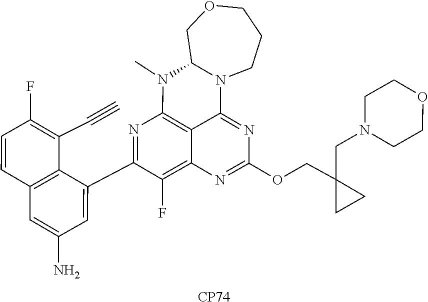

Compound 74 (CP74, confirmed) was synthesized in a manner similar to CP46 with CP9-6 and 74-a. MS: m/z: 628 [M+H]⁺.

### Example 76

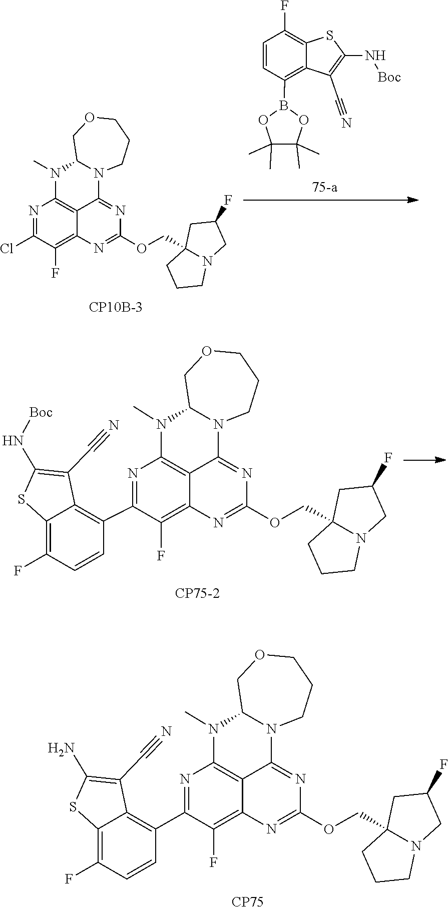

To a solution of CP10B-3 (64 mg), 75-a (64 mg), Cs₂CO₃ (137 mg) in toluene (10 mL) and water (2.5 mL) was added bis(diphenylphosphinophenyl)ether palladium (II) dichloride (10 mg). The mixture was purged with nitrogen followed by stirring at 105° C. for 16 hours. Upon completion, the mixture was diluted with EA and water. The separated organic layer was concentrated to give CP75-2 (133 mg). MS: m/z: 723 [M+1]⁺.

To a solution of CP75-2 (133 mg) in DCM (5 mL) was added TFA (1.5 mL). The reaction mixture was stirred at RT for 2 hours. After completion, the reaction mixture was concentrated. The residue was diluted with EA and saturated NaHCO₃. The separated organic layer was concentrated and purified to afford Compound 75 (CP75, confirmed). MS: m/z: 623 [M+H]⁺.

### Example 77

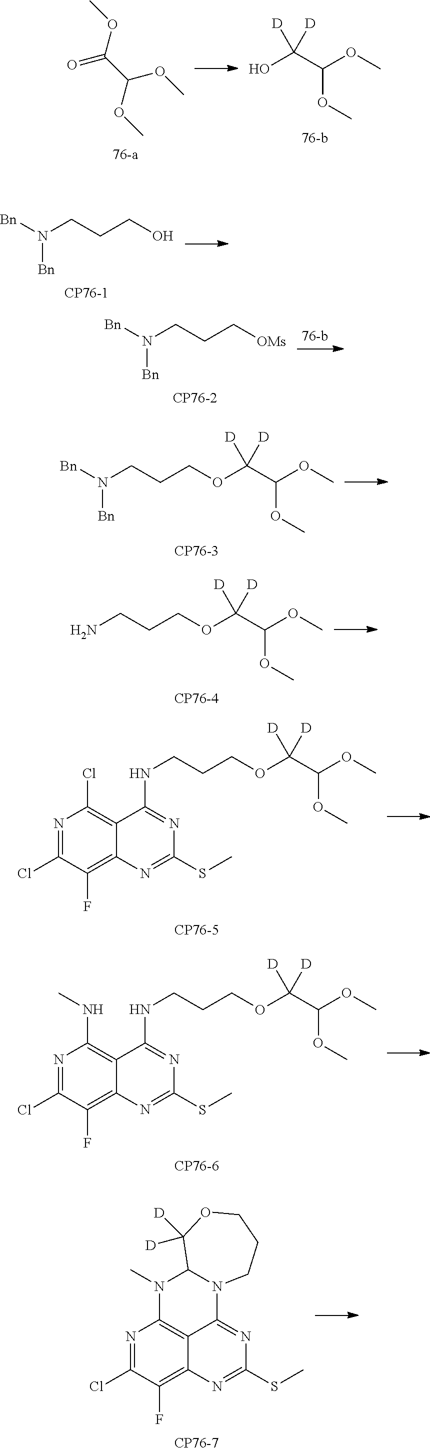

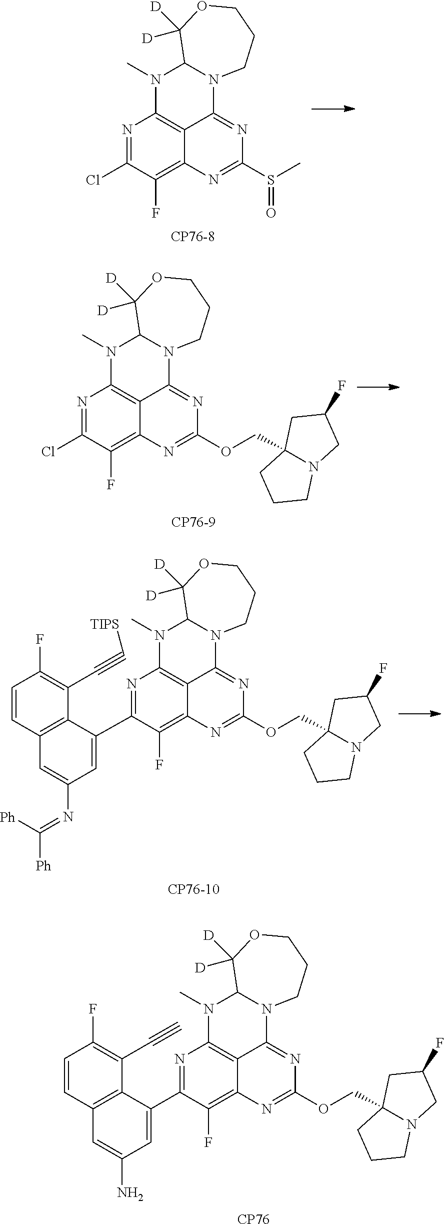

To a 0° C. solution of 76-a (5094 mg) in THF (50 mL) was added lithium aluminum deuteride (1508 mg). The mixture was stirred at 0° C. for 4 h, quenched with 1.5 mL water, 1.5 mL 15% NaOH, 4.5 mL water and a small amount of anhydrous Na₂SO₄. The mixture was filtered and the filtrate was concentrated to give 76-b (3285 mg). MS: m/z: 109 [M+H]⁺.

Compound 76 (CP76, confirmed) was synthesized in a manner similar to CP9 with CP76-b. MS: m/z: 618 [M+H]⁺.

### Example 78

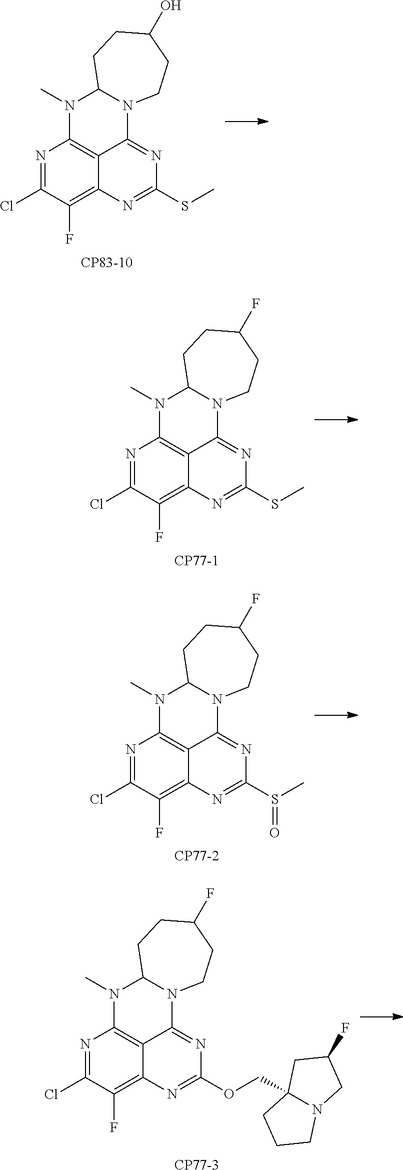

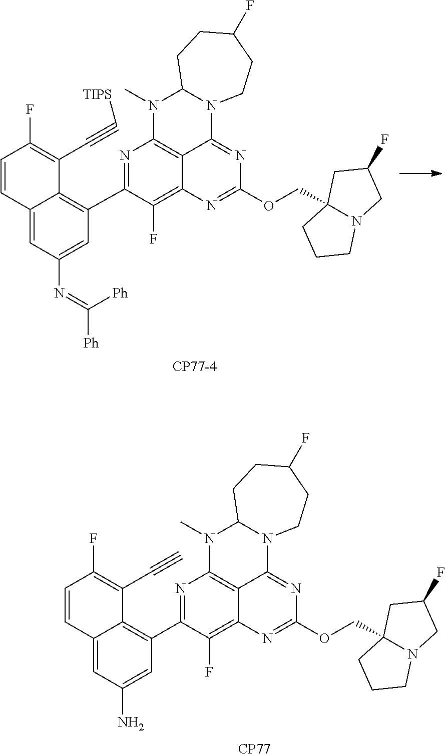

To a 0° C. solution of CP93-10 (0.698 g) in DCM (5 mL) was added DAST (0.493 g). The mixture was stirred at 0° C. for 1.5 h, quenched with sat. NaHCO₃ (aq.), extracted with DCM and post processed to give CP77-1 (0.206 g). MS: m/z: 372[M+H]⁺.

Compound 77 (CP77, 0.0013 g, TFA salt, confirmed) was synthesized in a manner similar to CP46 with CP77-1. MS: m/z: 632 [M+H]⁺.

### Example 79

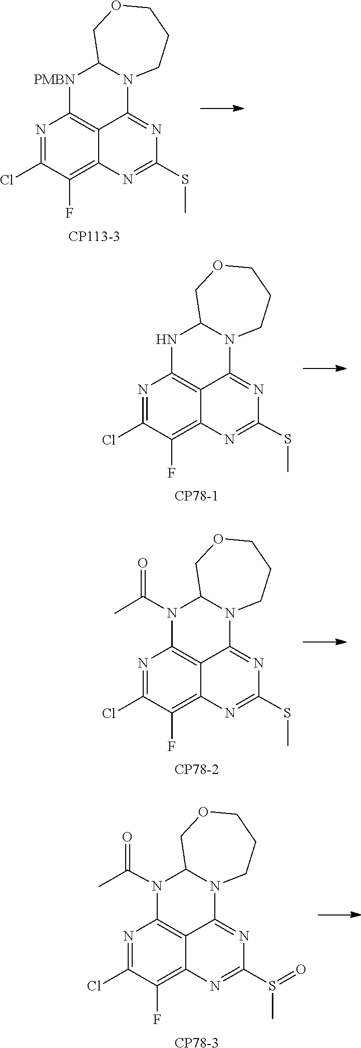

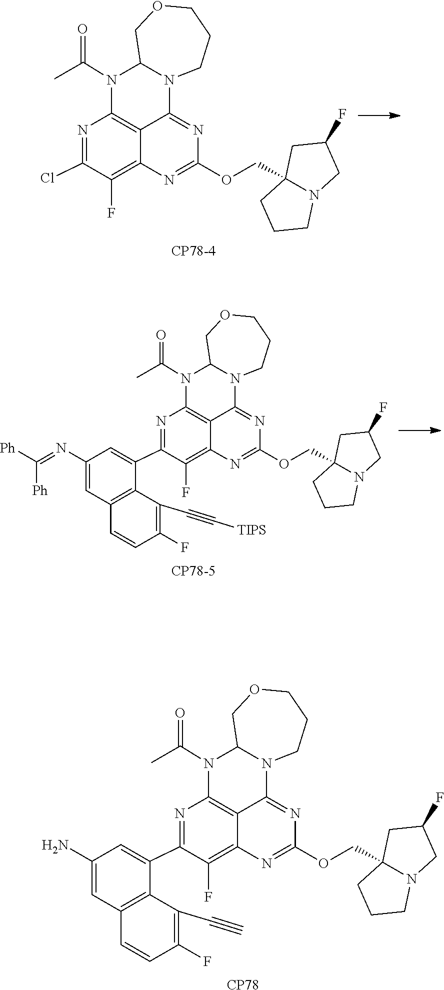

A mixture of CP113-3 (1.295 g) in trifluoroacetic acid (5 mL) was stirred at RT overnight. The reaction mixture was concentrated. The resulting mixture was diluted with PE and EA (10:1) and filtered. The filter cake was concentrated to give CP78-1 (1024 mg). MS: m/z: 342[M+H]⁺.

A mixture of CP78-1 (0.201 g) in acetic acid (2 mL) and acetic anhydride (1 mL) was stirred at 80° C. for 3 days, quenched with H₂O, extracted with DCM and MeOH (10:1) and post processed to give CP78-2 (206 mg). MS: m/z: 384 [M+H]⁺.

Compound 78 (CP78, confirmed) was synthesized in a manner similar to CP46 with CP78-2. MS: m/z: 644 [M+H]⁺.

### Example 80

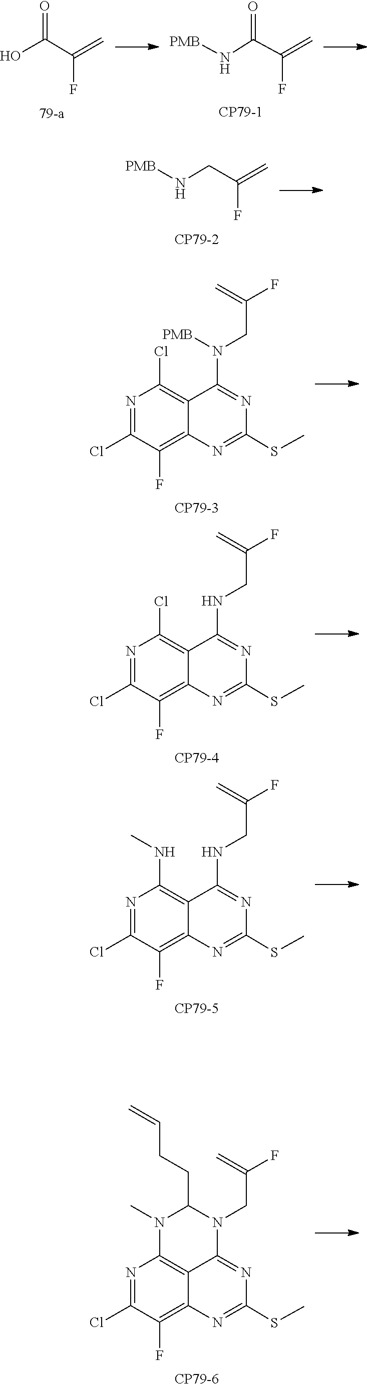

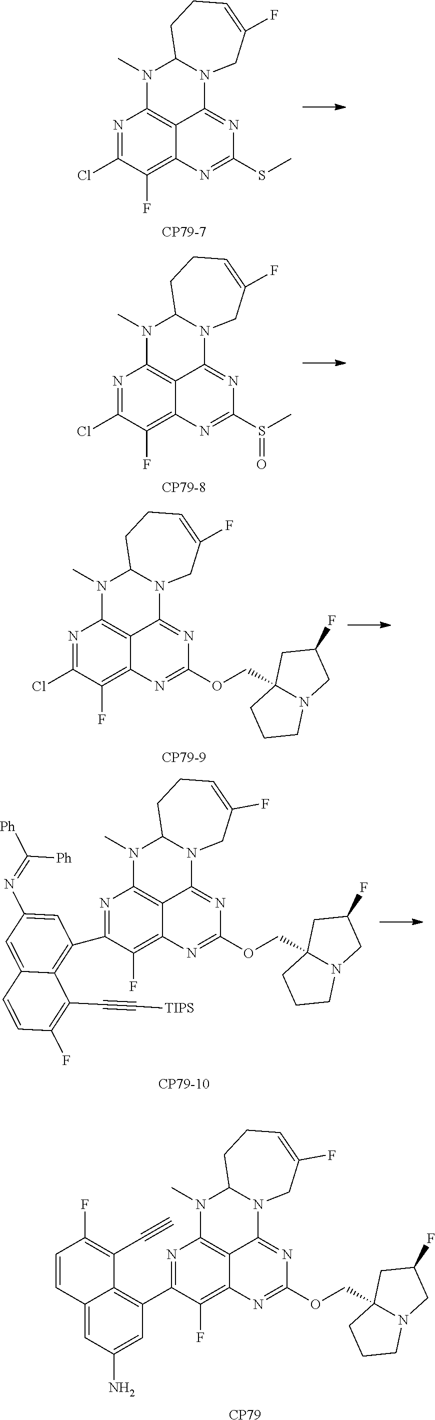

A solution of 79-a (3.58 g), 4-methoxybenzylamine (5.62 g,), DIEA (10.0170 g) and HATU (15.18 g) in DCM (80 mL) was stirred overnight at RT. The mixture was extracted with DCM and post processed to give CP79-1 (1.05 g). MS: m/z: 210[M+H]⁺.

To a 0° C. THF (10 mL) was added LAH (0.308 g). The mixture was stirred at 0° C. for 15 min, and aluminum chloride (1.071 g) was added. The mixture was stirred at 0° C. for 30 min, and CP79-1 (0.74 g) was added. The mixture was stirred at 0° C. for 30 min, quenched with NaOH (aq, 0.3 mL, 15%), extracted with DCM, washed with NH₄Cl, dried and concentrated to give CP79-2 (0.39 g). MS: m/z: 196[M+H]⁺.

A solution of INT6 (0.603 g) DIEA (1.4840 g) and POCl₃ (2.6320 g) in toluene (20 mL) was stirred at 80° C. for 1 h. The reaction was concentrated to give a mixture. To a 0° C. solution of the mixture and DIEA (2.6320 g) in DCM (5 mL) was added CP79-2 (0.39 g). The mixture was stirred at 0° C. overnight. The residue was concentrated and purified to give CP79-3 (238 mg). MS: m/z: 457[M+H]⁺.

A solution of CP79-3 (0.218 g) in TFA (3 mL) was stirred at 60° C. for 1 h. The mixture was concentrated to give CP79-4 (0.298 g). MS: m/z: 337[M+H]⁺.

Compound 79 (CP79, confirmed) was synthesized the procedure of CP71 with CP79-4. LCMS: m/z: 630[M+H]⁺.

### Example 81

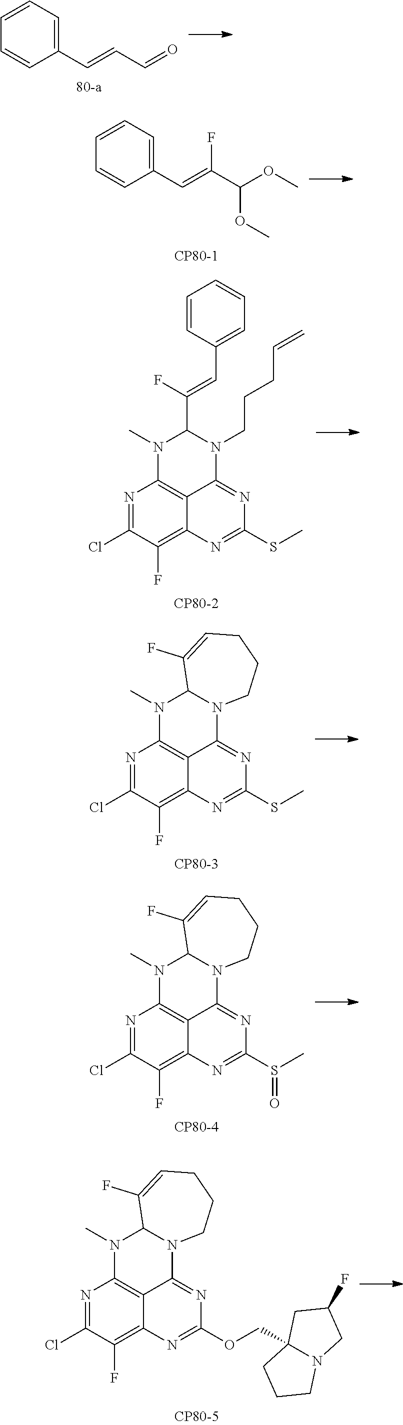 [SMILES: O=C/C=C/c1ccccc1 ; COC(OC)/C(F)=C/c1ccccc1 ; C=CCCCN1c2nc(SC)nc3c(F)c(Cl)nc(c23)N(C)C1/C(F)=C/c1ccccc1 ; CSc1nc2c3c(nc(Cl)c(F)c3n1)N(C)C1C(F)=CCCCN21 ; CN1c2nc(Cl)c(F)c3nc(S(C)=O)nc(c23)N2CCCC=C(F)C12 ; CN1c2nc(Cl)c(F)c3nc(OC[C@@]45CCCN4C[C@H](F)C5)nc(c23)N2CCCC=C(F)C12]

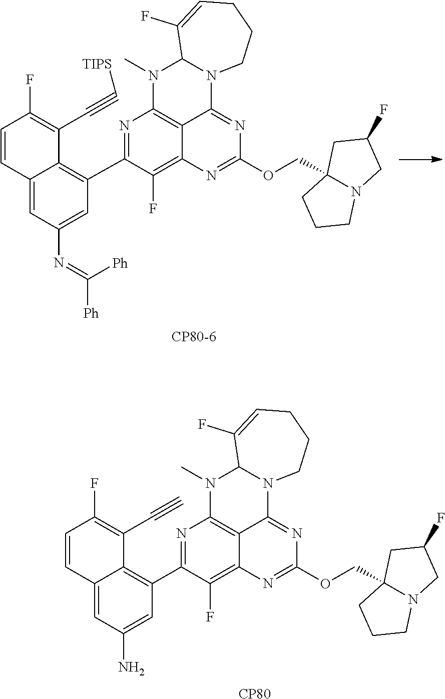

To a 0° C. solution of 80-a (5.01 g) and L-proline (3.66 g) in MeCN (50 mL) and methanol (50 mL) was added selectfluor (27.58 g). The mixture was stirred at 55° C. overnight. The mixture was concentrated and purified to give CP80-1 (3.284 g).

Compound 80 (CP80, confirmed) was synthesized in a manner similar to CP71 with CP80-1. MS: m/z: 630[M+H]⁺.

### Example 82

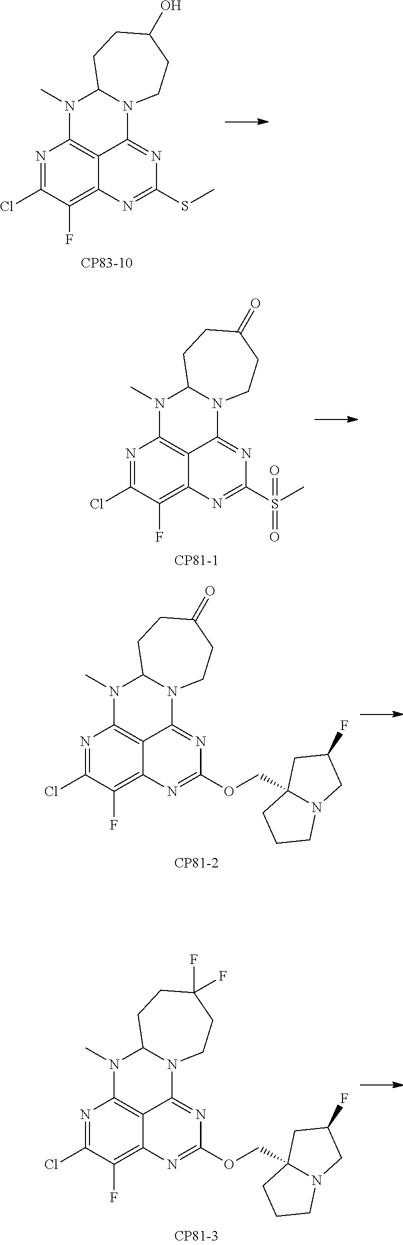 [SMILES: CSc1nc2c3c(nc(Cl)c(F)c3n1)N(C)C1CCC(O)CCN21 ; "CP81-1" ; CN1c2nc(Cl)c(F)c3nc(OC[C@@]45CCCN4C[C@H](F)C5)nc(c23)N2CCC(=O)CCC12 ; CN1c2nc(Cl)c(F)c3nc(S(C)(=O)=O)nc(c23)N2CCC(=O)CCC12 ; CN1c2nc(Cl)c(F)c3nc(OC[C@@]45CCCN4C[C@H](F)C5)nc(c23)N2CCC(F)(F)CCC12]

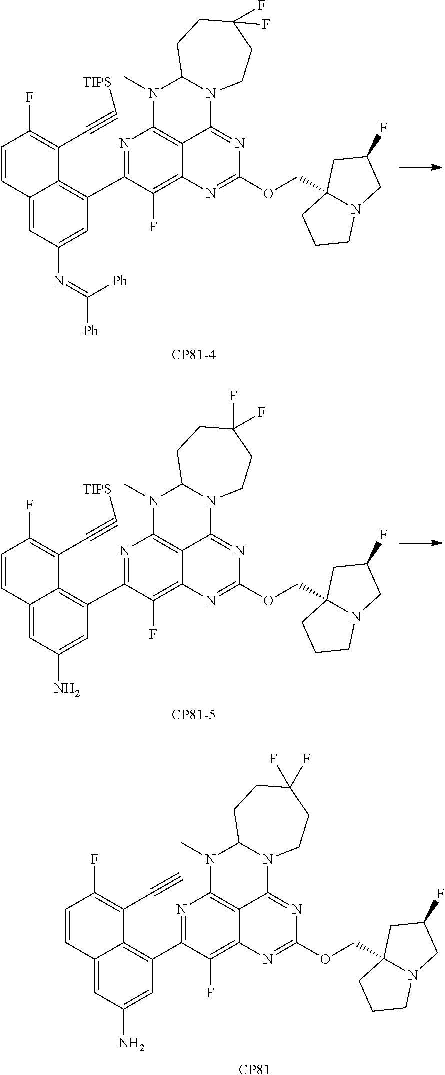

To a solution of CP83-10 (0.1 g) in DCM (10 mL) were added NaHCO₃ (29 mg) and Dess-Martin periodinane (139 mg) at RT and the resulting mixture was stirred for 1 h. The reaction was filtered and the filtrate was concentrated to give the crude product. The crude product was diluted with DCM (2 mL) and added m-CPBA (65 mg) at RT and stirred for 1 h. The residue was diluted with sat. sodium thiosulfate, extracted with DCM and post processed to afford CP81-1 (173 mg). The crude product was used in the next step directly. MS (ESI, m/z): 400[M+H]⁺.

A solution of molecular sieves, 4A, CP81-1 (172 mg) and DIEA (160 mg), INT14 (109 mg) in toluene (5 mL) was stirred at 80° C. for 16 hours under N₂. The reaction mixture was cooled to RT, concentrated and purified to afford CP81-2 (65 mg). MS (ESI, m/z): 479[M+H]⁺.

To a 0° C. solution of CP81-2 (0.052 g) in DCM (1 mL) was added DAST (0.5 mL) batch-wise over 2 min under N₂. The mixture was stirred at RT for 3 h, quenched with sat. NaHCO₃, extracted with DCM and post processed to afford CP81-3 (10 mg). MS (ESI, m/z): 501[M+H]⁺.

Compound 81 (CP81, confirmed) was synthesized in a manner similar to CP46 with CP81-3. MS (ESI, m/z): 650[M+H]⁺.

### Example 83

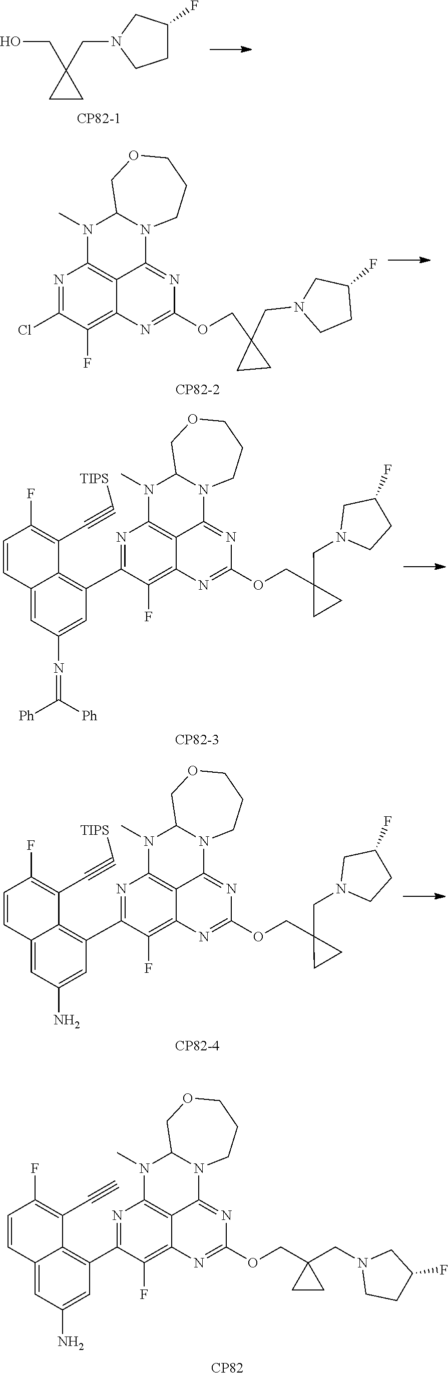

Compound 12 (CP12, confirmed) was synthesized m a manner similar to CP46 with CP82-1. MS: m/z: 630[M+H]⁺.

### Example 84

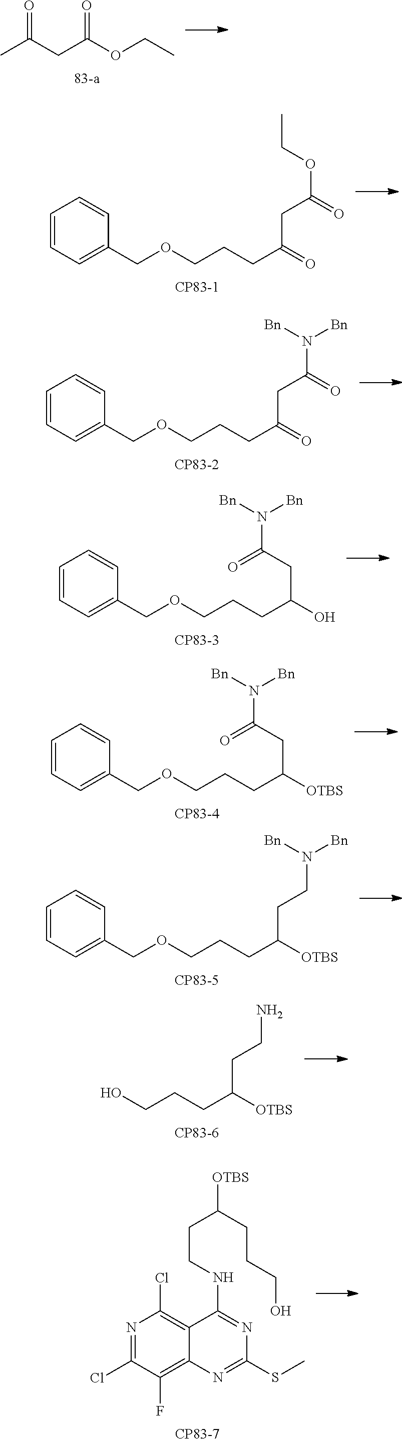

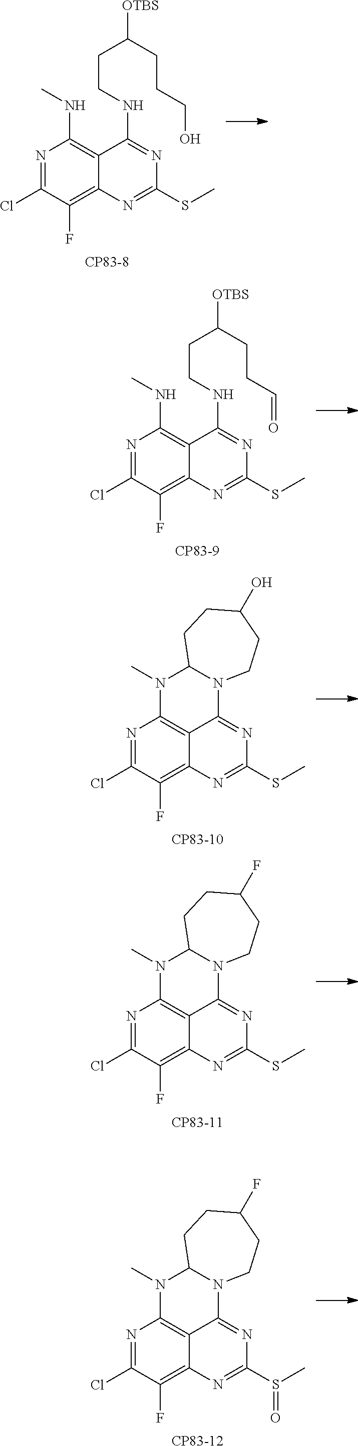

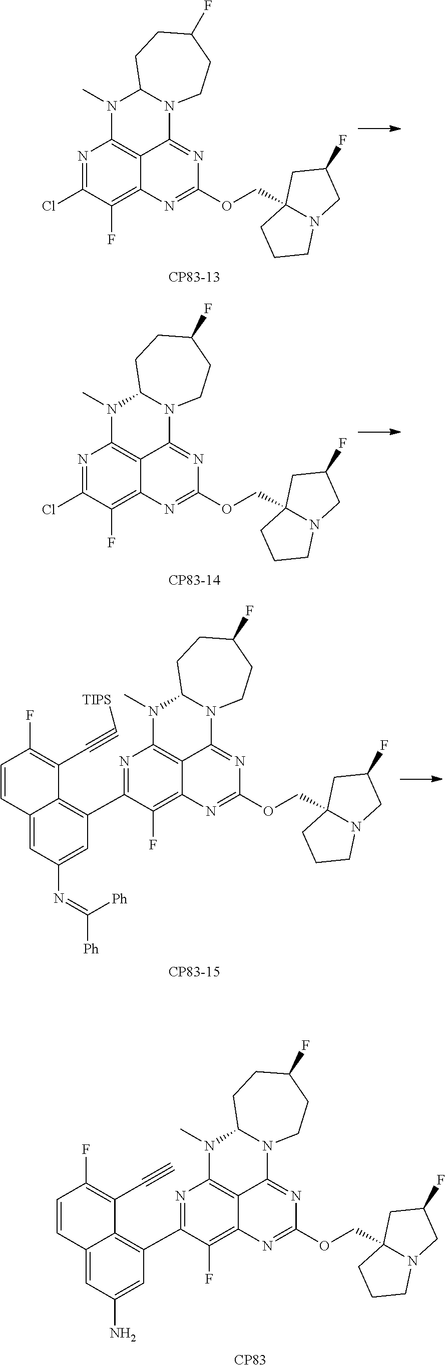

To a −20° C. solution of 83-a (50.04 g) in THF (400 mL) was added NaH (15.54 g). The mixture was stirred at −20° C. for 2 h. The reaction mixture was added n-BuLi (210 mL) and stirred at −20° C. for 1 h. The reaction mixture was added benzyl 2-bromoethyl ether (81.59 g) in THF (100 mL). The mixture was stirred at RT overnight, quenched with sat. NH₄Cl (aq.) and water, extracted with EA and post processed to give CP83-1 (92.29 g). MS: m/z: 265 [M+H]⁺.

A solution of CP83-1 (48.77 g) in dibenzylamine (200 mL) was stirred at 100° C. overnight under N₂, quenched with water, extracted with EA and post processed to give CP83-2 (76.66 g). MS: m/z: 416 [M+H]⁺.

To a 0° C. solution of CP83-2 (76.66 g) in THF (200 mL) was added NaBH₄ (12.74 g). The mixture was stirred at RT for 5 h, quenched with MeOH (20 mL) and water, extracted with EA and post processed to give CP83-3 (47.61 g). MS: m/z: 418 [M+H]⁺.

To a 0° C. solution of CP83-3 (41.16 g) and imidazole (27.32 g) in DMF (50 mL) was added of TBDMSCl (45.26 g). The mixture was stirred at RT for 2 h, quenched with water, extracted with EA and post processed to give CP83-4 (57.45 g). MS: m/z: 532 [M+H]⁺.

To a −40° C. solution of CP83-4 (57.45 g) in THF (400 mL) was added diisobutylaluminium hydride (250 mL). The mixture was stirred at −20° C.~−40° C. for 3 h, quenched with EA (300 mL) and potassium sodium tartrate solution. The mixture was warmed to RT and stirred vigorously for 4 h. When the solution was clear, the solution was extracted with EA, post processed to give CP83-5 (8.29 g). MS: m/z: 518 [M+H]⁺.

A solution of CP83-5 (8.29 g) Pd/C (4.07 g) and Pd(OH)₂/C (5.34 g) in IPA (200 mL) was stirred at 65° C. overnight under H₂. The mixture was filtered and the filtrate was collected and concentrated to give CP83-6 (3.989 g). MS: m/z: 248 [M+H]⁺.

A solution of INT6 (4.45 g) and phosphorus oxychloride (8 mL) and DIEA (8 mL) in toluene (50 mL) was stirred at 80° C. overnight. The reaction was concentrated to give a mixture. To a 0° C. solution of the mixture and DIEA (2 mL) in DCM (10 mL) was added CP83-6 (3.53 g). The mixture was stirred 0° C. for 1 h. and then purified to give CP83-7 (3.78 g). MS: m/z: 509 [M+H]⁺.

A solution of CP83-7 (3.59 g), methylamine hydrochloride (2.62 g) and DIEA (7.07 g) in DMA (20 mL) was stirred at 80° C. for 3 h, quenched with water, extracted with EA and post processed to give CP83-8 (4.447 g). MS: m/z: 504 [M+H]⁺.

To a −40° C. solution of CP83-8 (4.29 g) and TEA (7.97 g) in DCM (6 mL) and methyl sulfoxide (12 mL) was added of pyridine sulfur trioxide (4.74 g). The mixture was stirred at RT for 3 h, quenched with sat. Na₂S₂O₃ (aq.), extracted with DCM and post processed to give CP83-9 (2.301 g). MS: m/z: 502 [M+H]⁺.

A solution of CP83-9 (2.301 g) and p-TsOH·H₂O (0.914 g) in methyl sulfoxide (10 mL) was stirred at 80° C. overnight and then purified to give CP83-10 (0.726 g). MS: m/z: 370 [M+H]⁺.

To a 0° C. solution of CP83-10 (0.283 g) in DCM (5 mL) was added DAST (0.269 g). The mixture was stirred at 0° C. for 1.5 h, quenched with sat. NaHCO₃ (aq.), extracted with DCM and post processed to give CP33-11 (0.047 g). MS: m/z: 372[M+H]⁺.

Compound 83 (CP83, confirmed) was synthesized in a manner similar to CP46 with CP83-11. MS: m/z: 632[M+H]⁺.

### Example 85

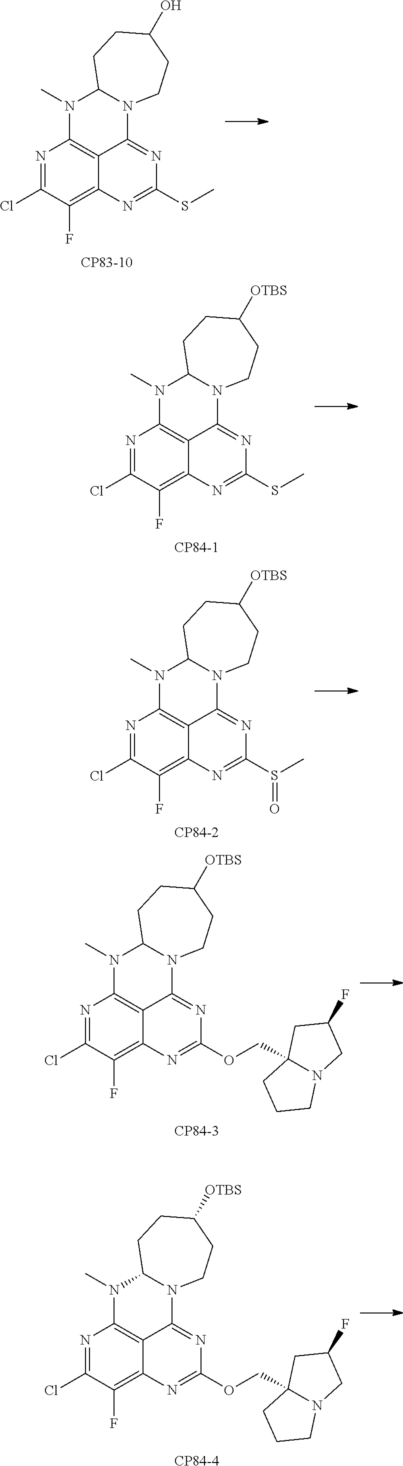

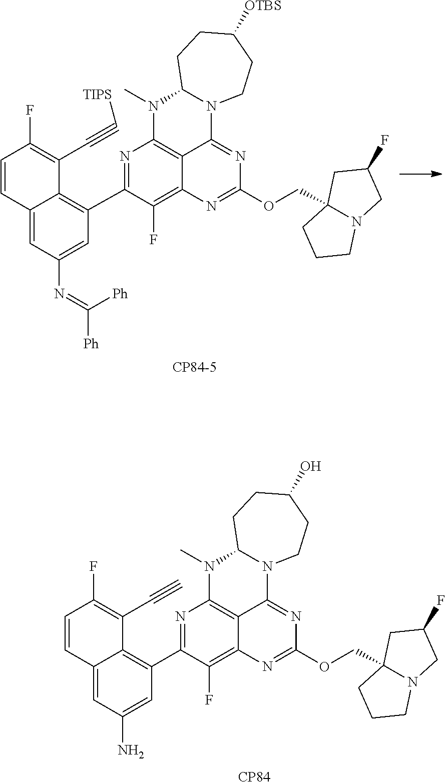

To a 0° C. solution of CP83-10 (0.101 g) and imidazole (0.140 g) was added TBDMSCl (0.138 g). The mixture was stirred at RT for 2 h, quenched with water, extracted with EA and post processed to give CP84-1 (0.057 g). MS: m/z: 484[M+H]⁺.

CP84-3 was synthesized in a manner similar to CP46-10 with CP84-1.

The racemate CP84-3 was separated by Prep-HPLC-Gilson with the conditions: Column, CHIRALPAK-IG. 20 mm×50 mm, 5 um; mobile phase, (Hex:DCM=3:1)(0.1% DEA)/EtOH (50:50); Flowing rate: 20 mL/min, 220 nm. This resulted in CP84-4A (the first eluting isomer, Retention time: 5.767 min) and CP84-4 (the second eluting isomer, Retention time: 7.067 min, 0.015 g). MS: m/z: 595[M+H]⁺.

A solution of CP84-4 (0.015 g). INT13 (0.038 g), cataCXium A Pd G3 (0.020 g) and Cs₂CO₃ (0.043 g) in toluene (5 mL) and water (1 mL) was stirred at 100° C. overnight under N₂. The mixture was added water, extracted with EA and post processed to give CP84-5 (0.026 g). MS: m/z: 1065[M+H]⁺.

Compound 84 (CP84, confirmed) was synthesized in a manner similar to CP83 with the CP84-4. MS: m/z: 630 [M+H]⁺.

### Example 86

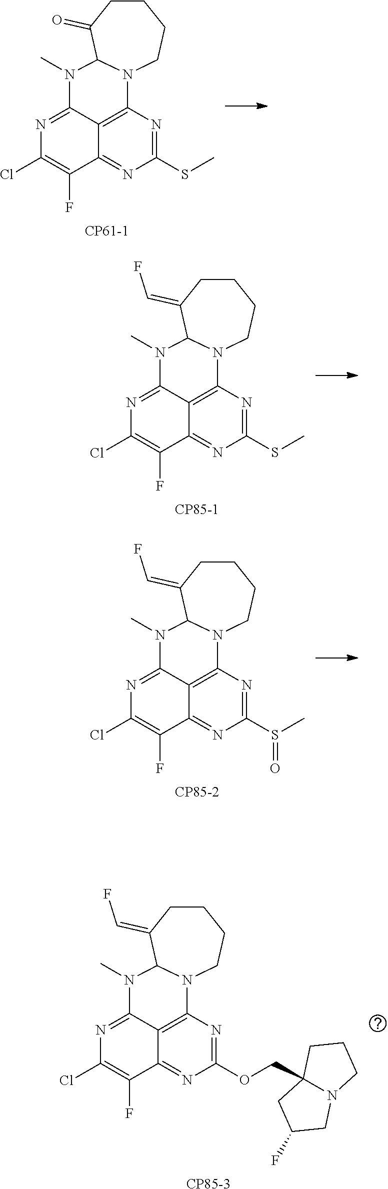

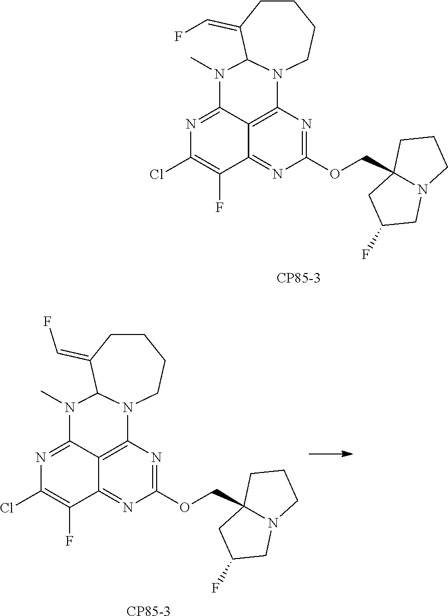 [SMILES: "CP85-3" ; CN1c2nc(Cl)c(F)c3nc(OC[C@@]45CCCN4C[C@H](F)C5)nc(c23)N2CCCC/C(=C\F)C12 ; "CP85-3" ; CN1c2nc(Cl)c(F)c3nc(OC[C@@]45CCCN4C[C@H](F)C5)nc(c23)N2CCCC/C(=C/F)C12]

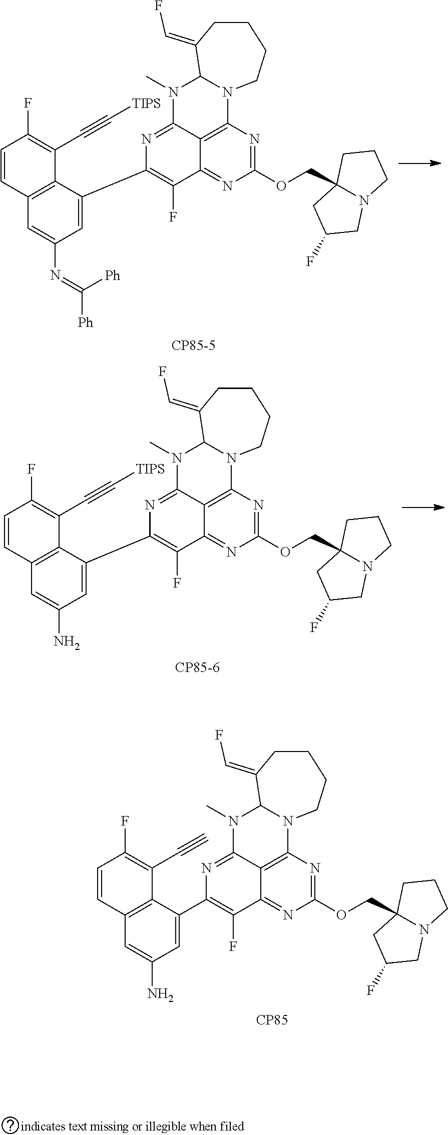

A solution of CP61-1 (0.508 g) in DMF (10 mL) was added fluoro methyl 2-pyridyl sulfone (0.286 g) and the resulting mixture was stirred at −50° C. under N₂. Potassium tert-butoxide (0.239 g) was added. The solution was stirred for 4 h at RT. The mixture was diluted with EA, washed with NH₄Cl (aq), H₂O (50 mL) and saturated NaCl (aq) and concentrated. 10 mL PE and 2 mL EA were added and the resulting mixture was stirred for 1 h. The mixture was filtrated to afford CP85-1 (346 mg). MS: m/z: 384[M+H]⁺.

A solution of CP85-1 (346 mg) in DCM (10 mL) was added m-CPBA (0.367 g) in portions at 0° C. and the resulting mixture was stirred at RT for 2 h. The mixture was diluted with DCM (50 mL) and washed with H₂O, saturated NaHCO₃ solution and saturated NaCl(aq). The organic layer was dried and concentrated to afford CP85-2 (365 mg, crude) which was used in the next step. MS: m/z: 400[M+H]⁺.

A solution of CP85-2 (365 mg), INT14 (0.243 g), DIEA (389 mg) in toluene (15 mL) was stirred at 85° C. for 16 h. The mixture was allowed to cool to RT, diluted with EA and washed with H₂O and saturated NaCl (aq). The organic layer was concentrated and purified by Prep-TLC with (DCM/MeOH=15:1) to give CP85-3 (174 mg) and CP85-4 (79 mg). MS: m/z: 495[M+H]⁺.

Compound 85 (CP85, confirmed) was synthesized in a manner similar to CP46 with CP85-3. LCMS: m/z: 644[M+H]⁺.

### Example 87

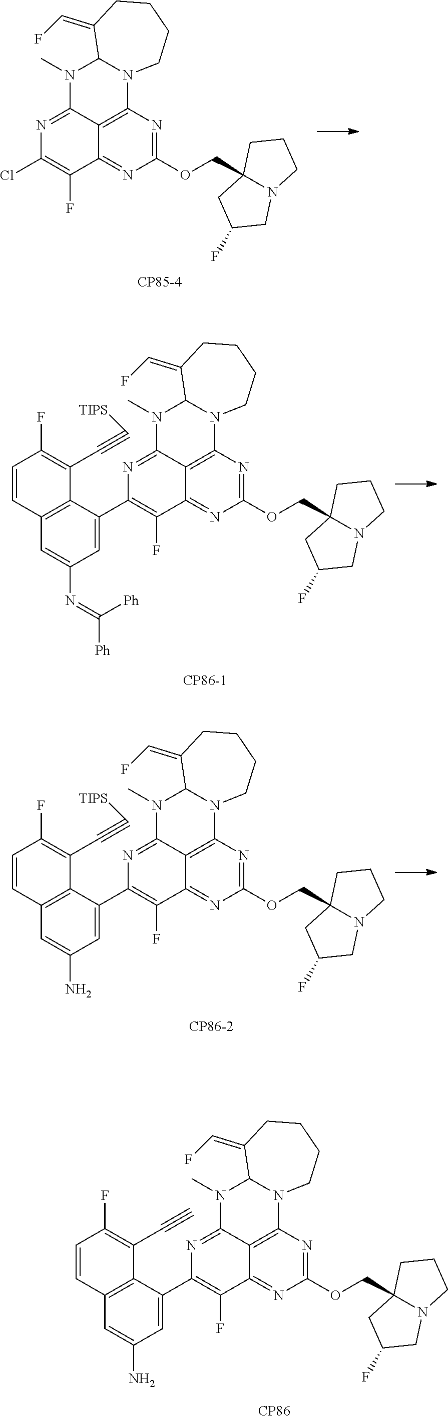

Compound 86 (CP86, confirmed) was synthesized in a manner similar to CP46 with CP85-4. MS: m/z: 644[M+H]⁺.

### Example 88

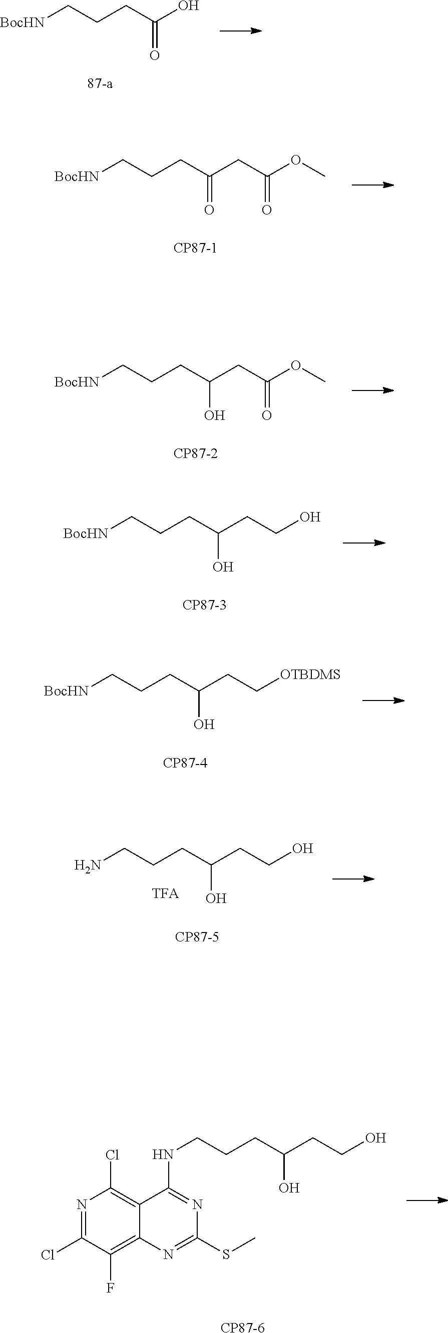

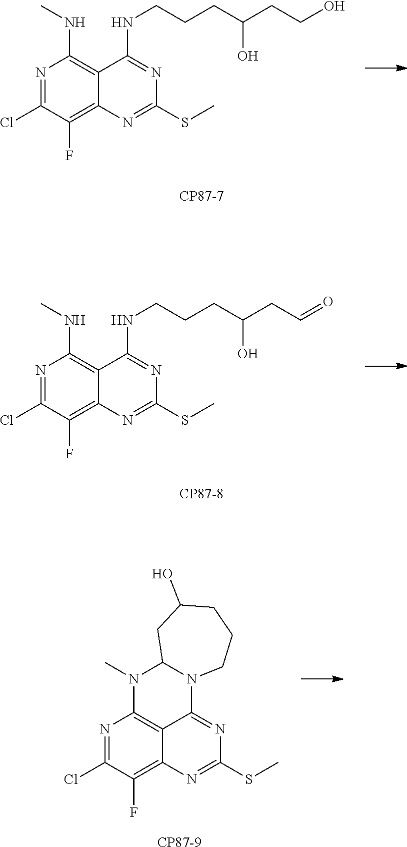

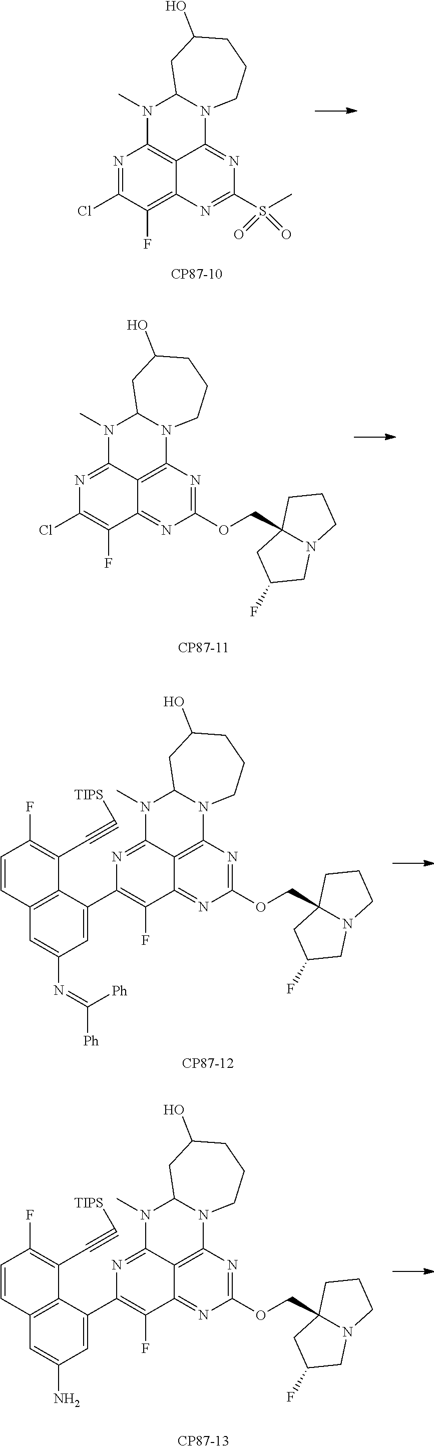

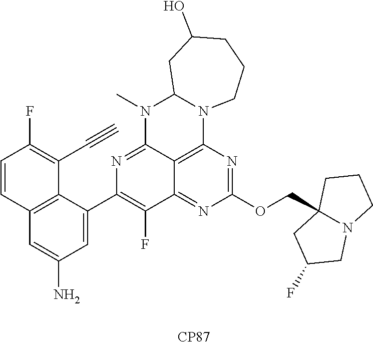 [SMILES: "CP87" ; C#Cc1c(F)ccc2cc(N)cc(-c3nc4c5c(nc(OC[C@@]67CCCN6C[C@H](F)C7)nc5c3F)N3CCCC(O)CC3N4C)c12]

A solution of 87-a (34.11 g) in DMF (350 mL) was added carbonyl diimidazole (39.17 g) and the resulting mixture was stirred at RT for 16 h. Potassium 3-methoxy-3-oxopropanoate (39.34 g), magnesium chloride (47.90 g) and TEA (51.57 g) were added at 0° C. The solution was stirred for 20 h at RT. The solution was filtered, diluted with water, extracted with EA and post processed to afford CP87-1 (40.33 g). MS: m/z: 260 [M+H]⁺.

A solution of CP87-1 (46.4720 g) in MeOH (400 mL) was added NaBH₄ (2.300 g) and the resulting mixture was stirred at 0° C. for 3 h. The solution was diluted with EA and post processed to afford CP87-2 (34.04 g). MS: m/z: 262 [M+H]⁺.

A solution of CP87-2 (4.11 g) in THF (50 mL) was added lithium aluminum hydride (633 mg) and the resulting mixture was stirred 2 h at 0° C., diluted with THF (20 mL), quenched with water (0.6 mL) and 15% solution of sodium hydroxide and water (1.8 mL). The solution was filtered, concentrated and purified to afford CP87-3 (3506 mg). MS: m/z: 234 [M+H]⁺.

To a solution of CP87-3 (11.765 g) in DCM (120 mL) were added TEA (7.6541 g), TBDMSCl (7.6005 g) and DMAP (616.061 mg) and the resulting mixture was stirred overnight at RT, diluted with DCM and post processed to give CP87-4 (12.691 g). MS: m/z: 348 [M+H]⁺.

To a solution of CP87-4 (8.557 g) in DCM (90 mL) was added trifluoroacetic acid (40 mL) at 0° C. and the resulting mixture was stirred at RT for 3 h and then concentrated to afford CP87-5 (22.433 g, crude) which was used in the next step. MS: m/z: 134 [M+H]⁺.

Compound 87 (CP87, confirmed) was synthesized in a manner similar to CP47 with CP87-5. MS: m/z: 630 [M+H]⁺.

### Example 89

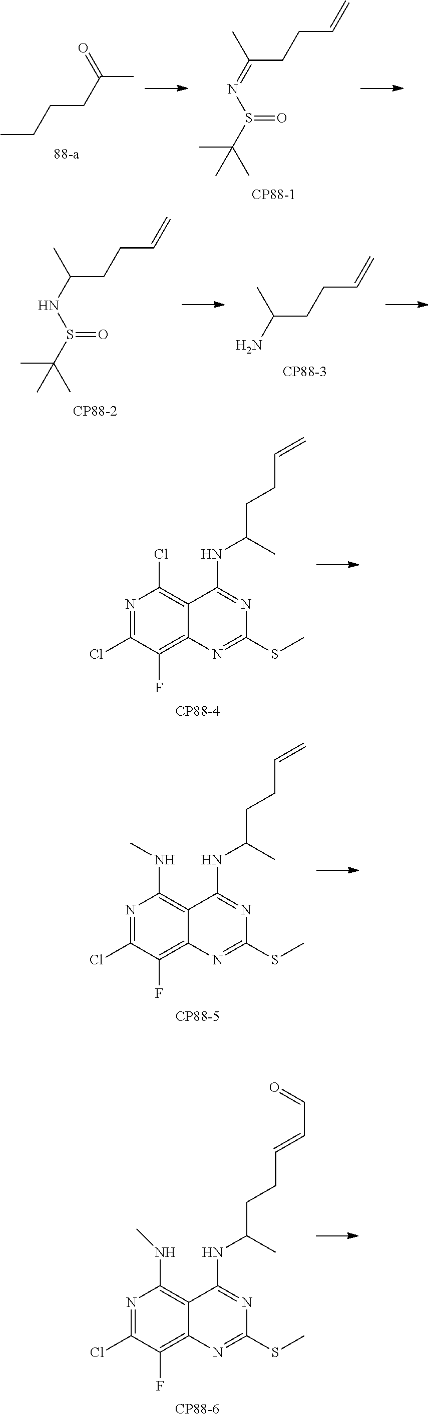

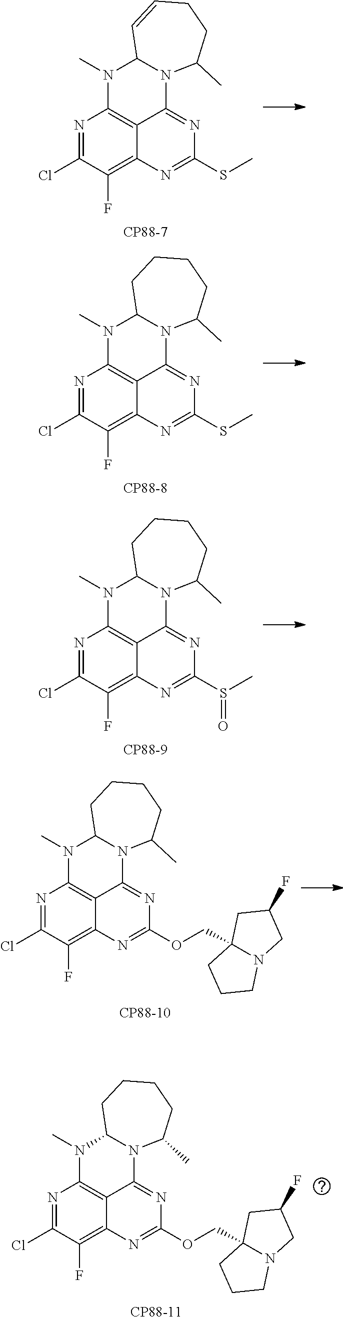

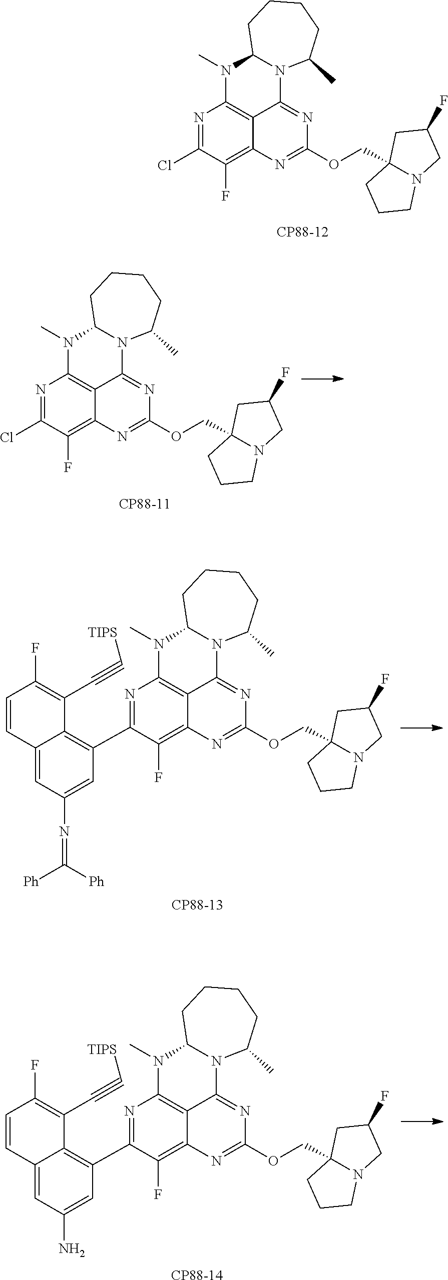

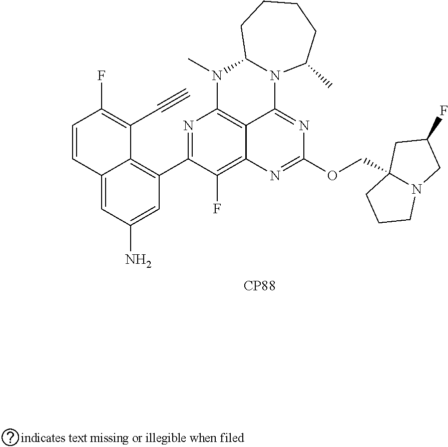 [SMILES: "CP88" ; C#Cc1c(F)ccc2cc(N)cc(-c3nc4c5c(nc(OC[C@@]67CCCN6C[C@H](F)C7)nc5c3F)N3[C@@H](C)CCCC[C@H]3N4C)c12]

A solution of 88-a (5.01 g), 2-methylpropane-2-sulfinamide (6.88 g), titanium ethoxide (18.91 g) in THF (150 mL) was stirred overnight at 65° C. The reaction was concentrated to afford CP88-1 (10.2 g) which was used in the next step. MS: m/z: 202[M+H]⁺.

A solution of CP88-1 (10.2 g) in methanol (80 mL) was added NaBH₄ (3.773 g) in portions at 0° C. and the resulting mixture was stirred for 0.5 h at this temperature, quenched with water and the white solid was filtered out. The organic liquid of the filtration was concentrated and the solution was extracted with EA. The organic layer was post processed to afford CP8&-2 (4.744 g). MS: m/z: 204[M+H]⁺.

A solution of CP88-2 (4.744 g) in dichloromethane (50 mL) was added hydrogen chloride (15 mL). The solution was stirred at RT for 2 h. The reaction was concentrated. The residue was dissolved in MTBE, concentrated. MTBE was added to the residue at 0° C. The solids were collected by filtration to afford CP8S-3 (1.784 g). MS: m/z: 100 [M+H]⁺.

CP88-5 was synthesized in a manner similar to INT15 with CP88-3.

A solution of CP8&-5 (593 mg). Grubb's second generation catalyst (103 mg), 3,3-dimethoxyprop-1-ene (461 mg) in DCM (10 mL) was stirred at 45° C. for 16 h under N₂. The mixture was allowed to cool to RT, concentrated and purified to give CP88-6 (453 mg). MS: m/z: 384[M+H]⁺.

CP88-10 was synthesized in a manner similar to CMS-10 with CP8S-6.

CP88-10 was separated by Prep-HPLC-Gilson with the conditions: Column, CHIRAL ART Cellulose-SC, 20 mm×250 mm, 5 um); mobile phase, (Hex:DCM=3:1)(0.1% DEA)/EtOH (50:50); Flowing rate: 15 mL/min, 220 nm. This resulted in CP88-11 (the second eluting isomer, retention time 6.053 min, 104 mg). MS: m/z: 479[M+H]⁺.

Compound 88 (CP88, confirmed) was synthesized in a manner similar to CP46 with CP88-11. MS: m/z: 628[M+H]⁺.

### Example 90

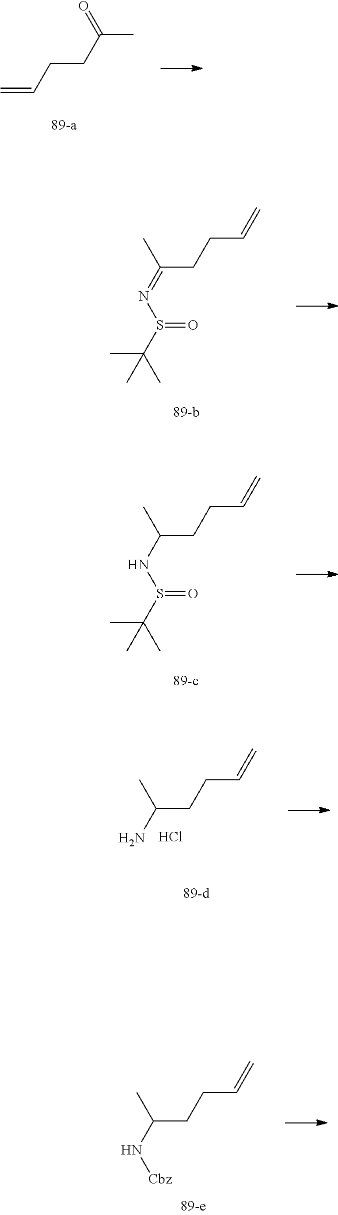

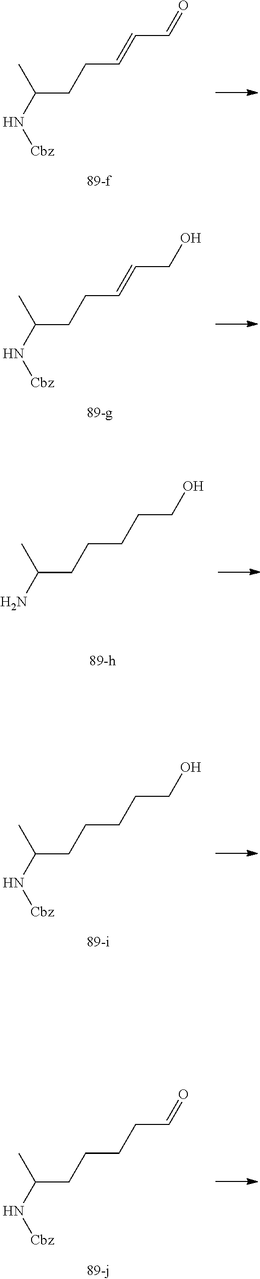

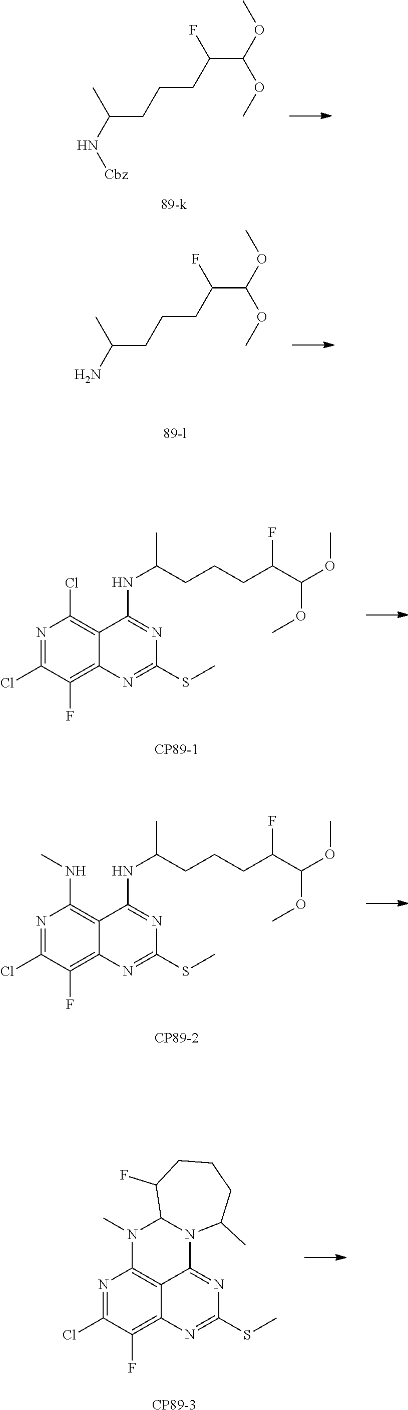

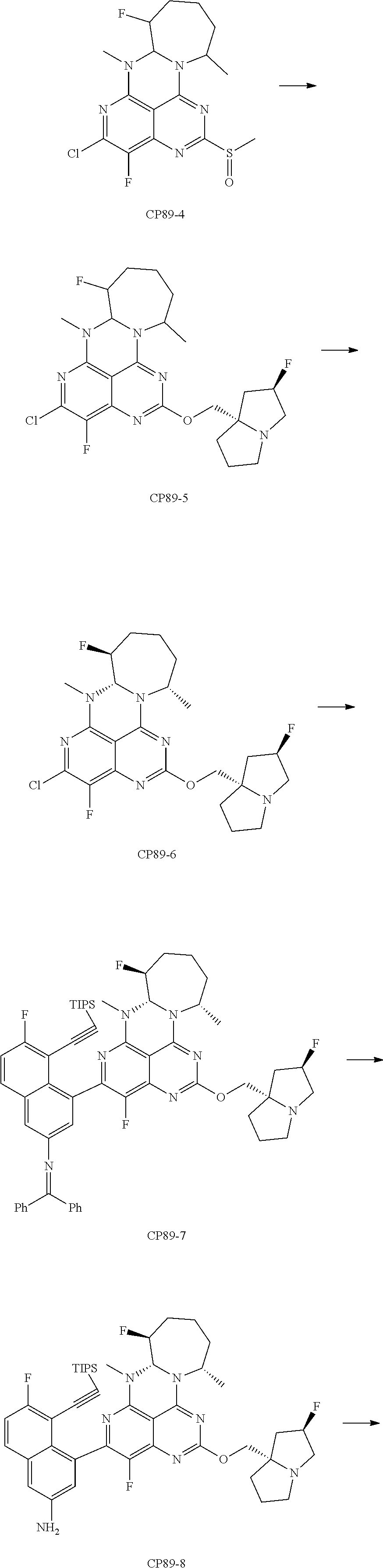

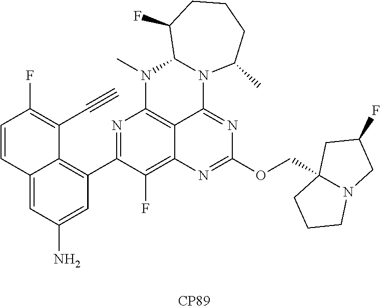

CP89-d was synthesized in a manner similar to CP8&3 with 89-a.

Cbz-Cl (3.545 g) was dropped into a solution of 89-d (2.327 g) and TEA (4.456 g) in DCM (50 mL) at 0° C. The solution was stirred at RT for 16 h under N₂. The mixture was diluted with NaCl solution and extracted with EA. The organic layer was post processed to give 89-e (2.975 g). MS: m/z: 234[M+H]⁺.

CP88-f was synthesized in a manner similar to CP88-6 with CP89-e.

NaBH₄ (0.743 g) was dropped into a solution of 89-f (3.364 g) in methanol (150 mL) at 0° C. The solution was stirred at RT for 3 h under N₂, quenched with water, extracted with EA and post processed to give 89-g (3.14 g). MS: m/z: 264[M+H]⁺.

A solution of 89-g (3.14 g) and Pd/C (604 mg) in THE (80 mL) was stirred at RT for 15 h under H₂. The mixture was directly to give 89-h (1.565 g). MS: m/z: 132[M+H]⁺.

Cbz-Cl (2.569 g) was dropped into a solution of 89-h (1.565 g) and TEA (1.906 g) in THF (50 mL) at 0° C. The solution was stirred at RT for 15 h under N₂. The solution was filtered and the filtrate was diluted with water, extracted with EA and post processed to give 89-1 (1.37 g). MS: m/z: 266[M+H]⁺.

A solution of 89-1 (1.37 g) and Pd/C (1.63 g) in DCM (50 mL) was stirred at RT for 5 h under N₂. The solution was filtered and the filtrate was concentrated and purified to give 89-j (893 mg). MS: m/z: 264[M+H]⁺.

A solution of 89-j (893 mg) and 1-chloromethyl-4-fluoro-1,4-diazoniabicyclo[2.2.2]octane bis(tetrafluoroborate)(2.541 g), L-proline (85 mg) in ACN (15 mL) and methanol (15 mL) was stirred at 65° C. for 16 h under N₂. The mixture was concentrated and purified to give 89-k (1.016 g). MS: m/z: 328[M+H]⁺.

A solution of 89-k (1.016 g) and Pd/C (0.338 g) in THF (30 mL) was stirred at RT for 2 h under H₂. The mixture was directly to give 89-1 (599.744 mg). MS: m/z: 194[M+H]⁺.

Compound 89 (CP89, confirmed) was synthesized in a manner similar to CP46 with CP89-1. MS: m/z: 646[M+1]⁺.

### Example 91

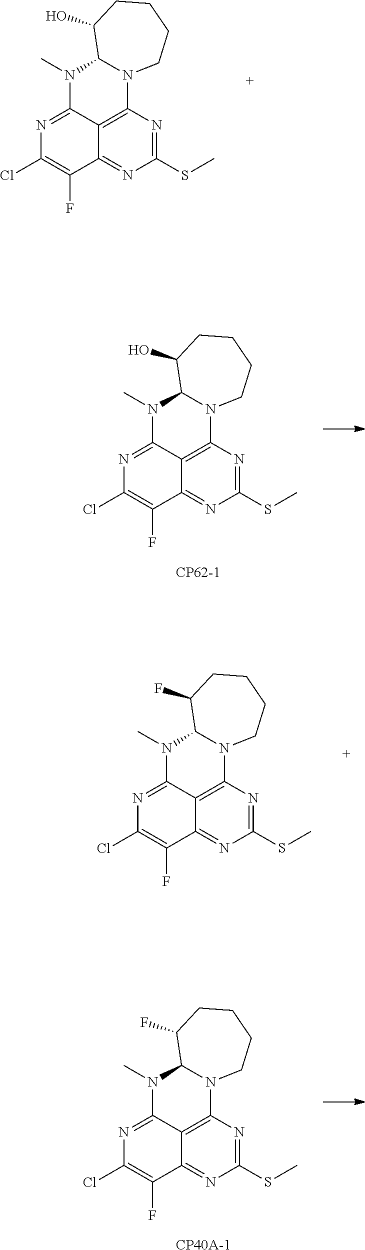 [SMILES: CSc1nc2c3c(nc(Cl)c(F)c3n1)N(C)[C@@H]1[C@H](O)CCCCN21 ; "CP62-1" ; CSc1nc2c3c(nc(Cl)c(F)c3n1)N(C)[C@H]1[C@@H](O)CCCCN21 ; CSc1nc2c3c(nc(Cl)c(F)c3n1)N(C)[C@@H]1[C@@H](F)CCCCN21 ; "CP40A-1" ; CSc1nc2c3c(nc(Cl)c(F)c3n1)N(C)[C@H]1[C@H](F)CCCCN21]

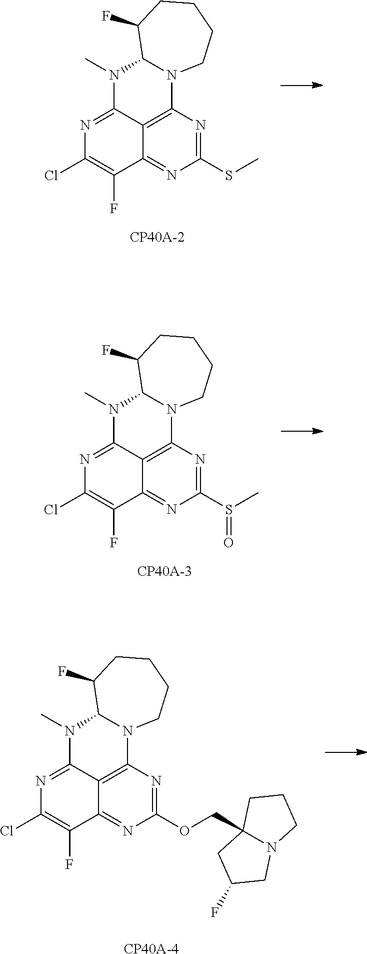 [SMILES: "CP40A-4" ; "CP40A-2" ; "CP40A-3" ; CN1c2nc(Cl)c(F)c3nc(OC[C@@]45CCCN4C[C@H](F)C5)nc(c23)N2CCCC[C@H](F)[C@@H]12 ; CN1c2nc(Cl)c(F)c3nc(S(C)=O)nc(c23)N2CCCC[C@H](F)[C@@H]12 ; CSc1nc2c3c(nc(Cl)c(F)c3n1)N(C)[C@@H]1[C@@H](F)CCCCN21]

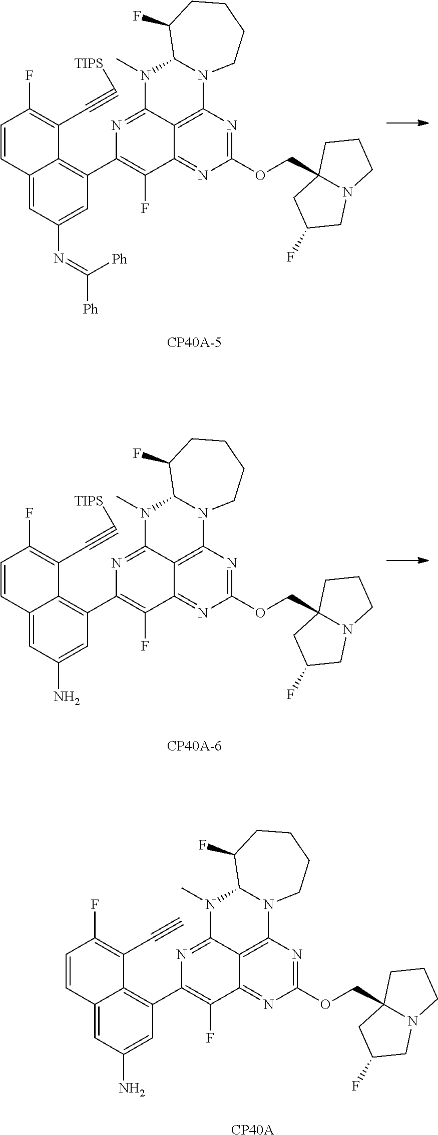

A solution of CP62-1 (2.64 g) in THF (200 mL) and DCM (00 mL) was added DAST (1.69 g) in dropwise at 0° C. under N₂ and stirred for 2 h at RT, quenched with saturated NaHCO₃ solution (100 mL), extracted with DCM (300 mL), dried, concentrated and purified by silica gel column with (PE/EA=5/1) to afford CP40A-1 (2.23 g).

CP40A-1 was separated by chiral-HPLC with the conditions: CHIRALPAK-IG, 20 mm×250 mm, Sum; mobile phase, Hex (0.1% DEA)/EtOH (70:30); Flowing rate: 20 mL/min to afford CP40A-2 (the first eluting isomer, Retention time: 6.3 min. 0.58 g). MS: m/z: 372[M+H]⁺.

Compound 40A (CP40A, confirmed) was synthesized the procedure of CP46 with CP40A-2 and purified with the condition: Prep-HPLC (YMC-Triart C18-S12 nm, 50 mm×250 mm, 7 um, A: 0.05% NH₃·H₂O in water, B: CH₃CN, Gradient: 35% B to 77% B in 36 min at a flow rate of 70 mL/min, 224 nm). LCMS: m/z: 632[M+H]⁺.

### Example 92

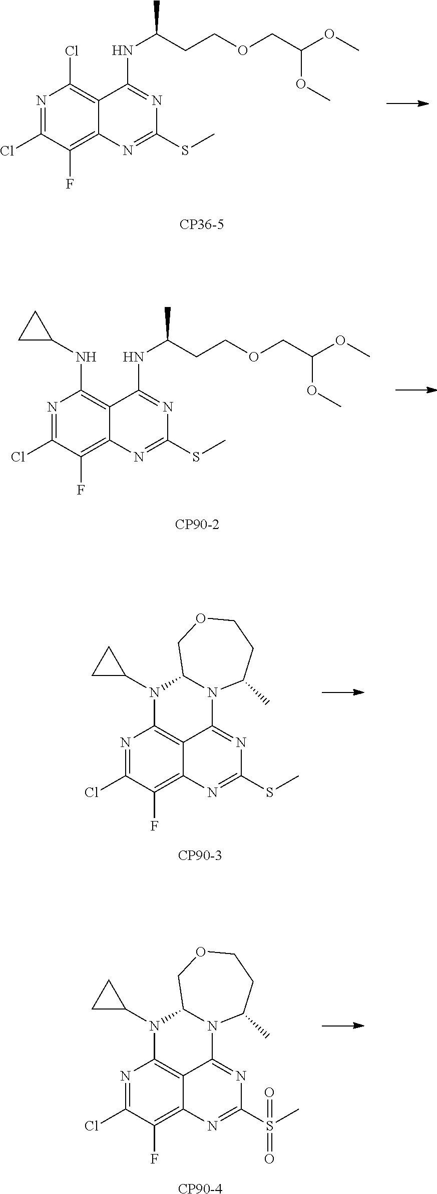

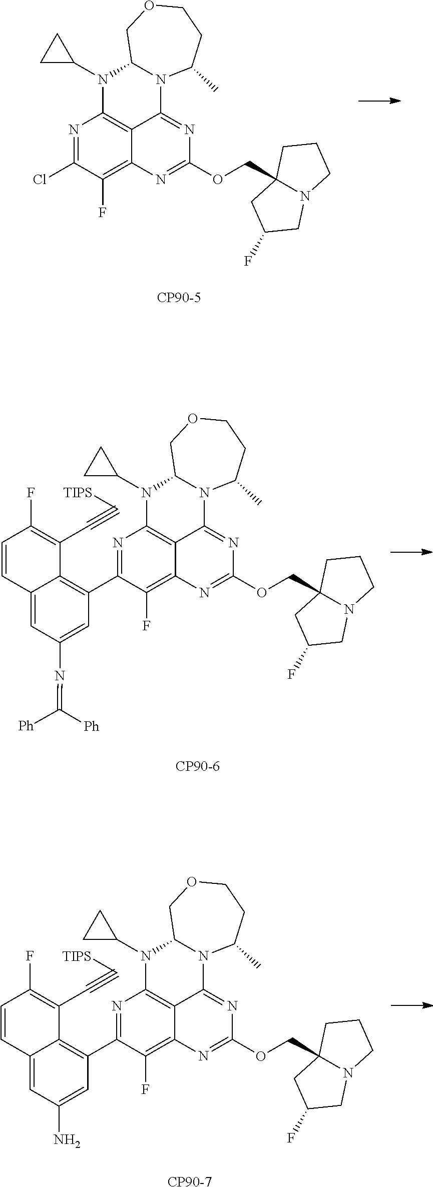

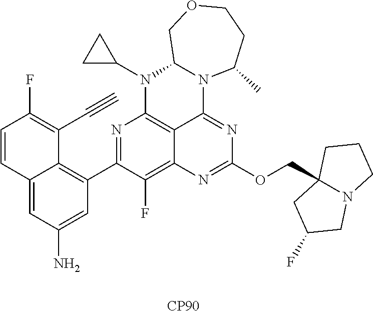 [SMILES: "CP90" ; C#Cc1c(F)ccc2cc(N)cc(-c3nc4c5c(nc(OC[C@@]67CCCN6C[C@H](F)C7)nc5c3F)N3[C@@H](C)CCOC[C@H]3N4C3CC3)c12]

Compound 90 (CP90, confirmed) was synthesized in a manner similar to CP46. MS: m/z: 656[M+1]⁺.

### Example 93

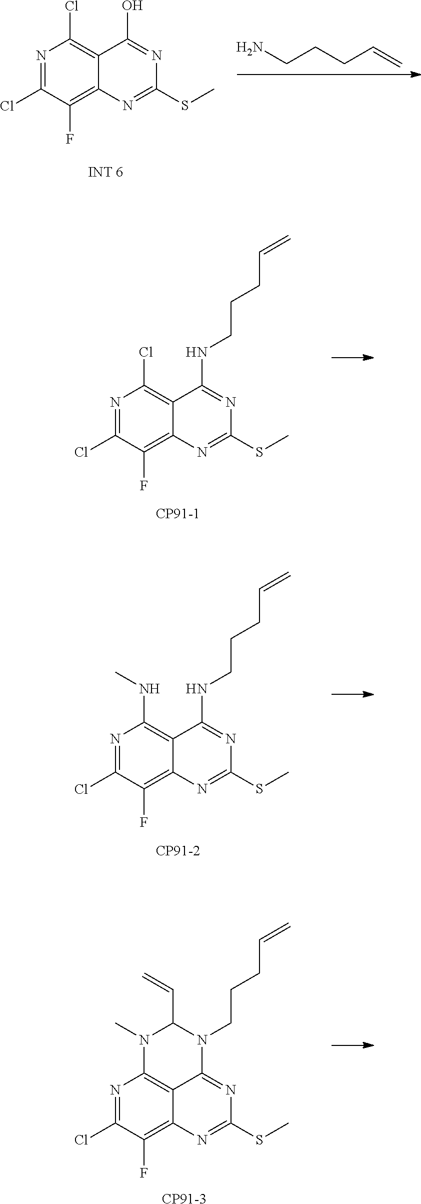

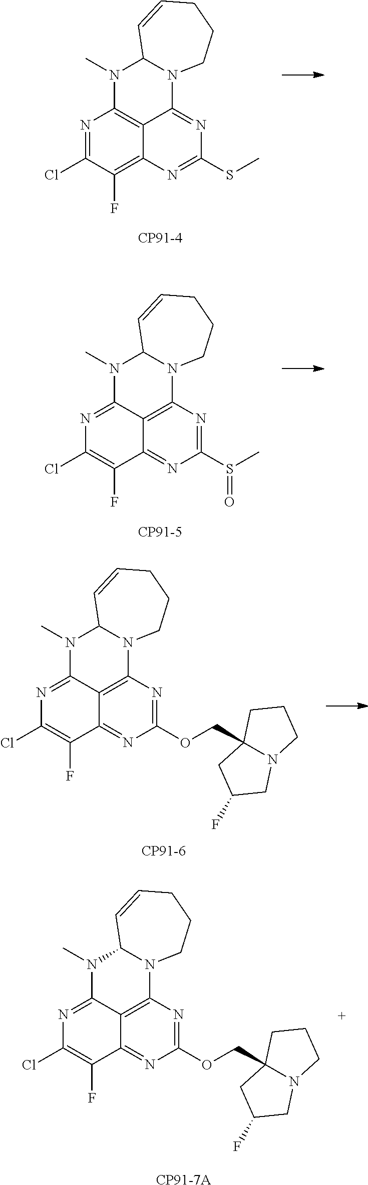

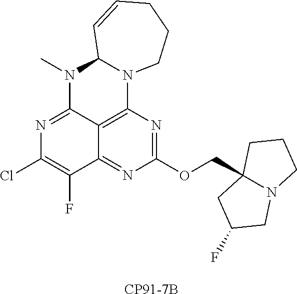 [SMILES: CN1c2nc(Cl)c(F)c3nc(OC[C@@]45CCCN4C[C@H](F)C5)nc(c23)N2CCCC=C[C@H]12 ; "CP91-7B"]

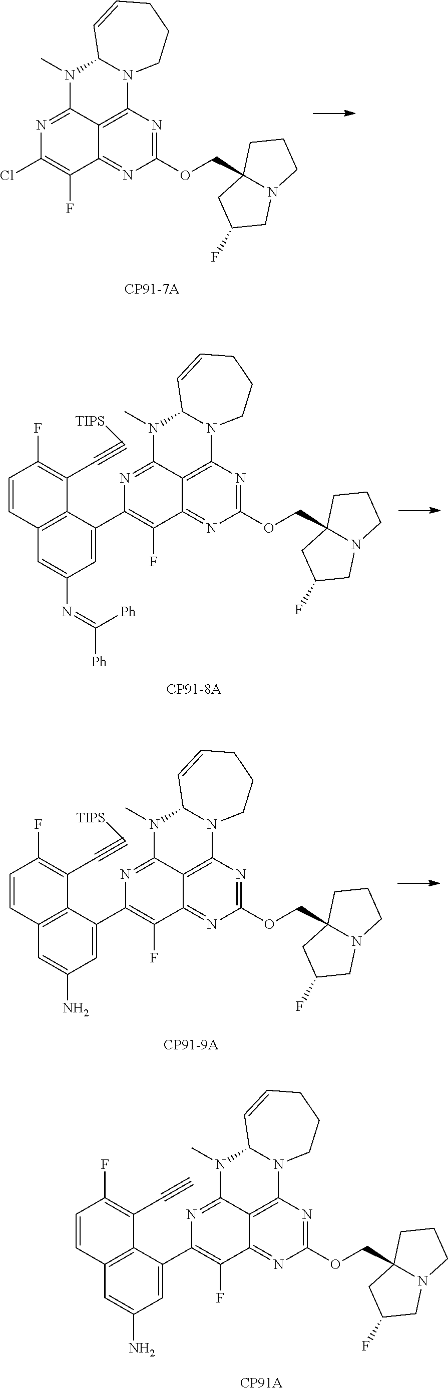

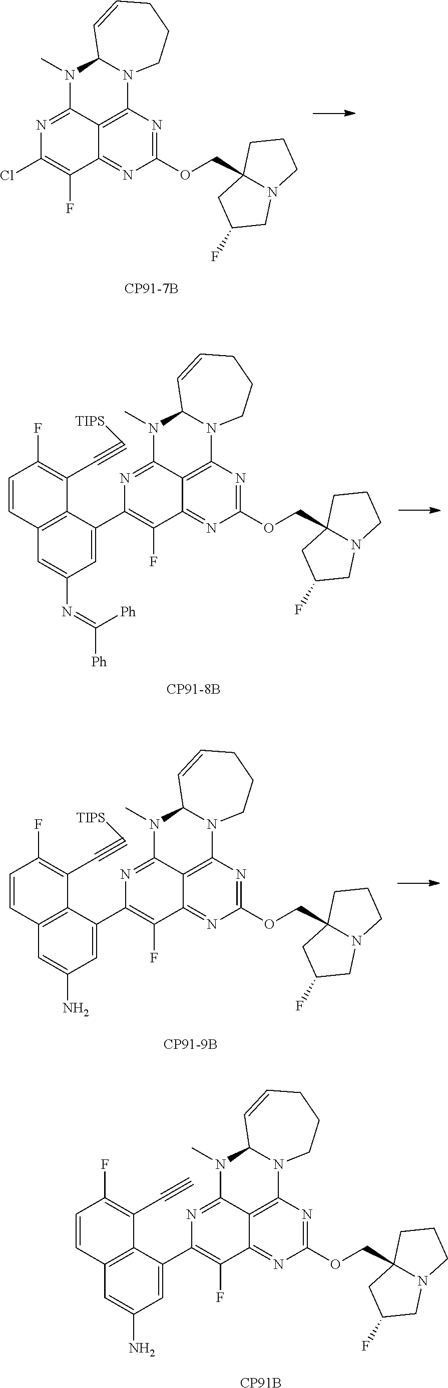

CP91-6 was synthesized in a manner similar to CP71-6.

CP91-6 was separated by Prep-HPLC-Gilson with the conditions: Column, CHIRAL ART Cellulose-SC, 20 mm×250 mm, 5 um; mobile phase, (Hex:DCM=3:1)(0.1% DEA)/EtOH (50:50); Flowing rate: 15 mL/min, 220 nm. This resulted in CP91-7A (41 mg, the first eluting isomer, Retention time: 6.903 min) and CP91-7B (50 mg, the second eluting isomer, Retention time: 7.681 min). MS: m/z: 463[M+1]⁺.

Compound 91A (CP91A, 26 mg, confirmed) was synthesized by the similar procedure of the synthesis of CP46 with CP91-7A. MS: m/z: 612[M+1]⁺.

Compound 91B (CP91B, 38 mg, confirmed) was synthesized by the similar procedure of the synthesis of CP46 with CP91-7B. MS: m/z: 612[M+1]⁺.

### Example 94

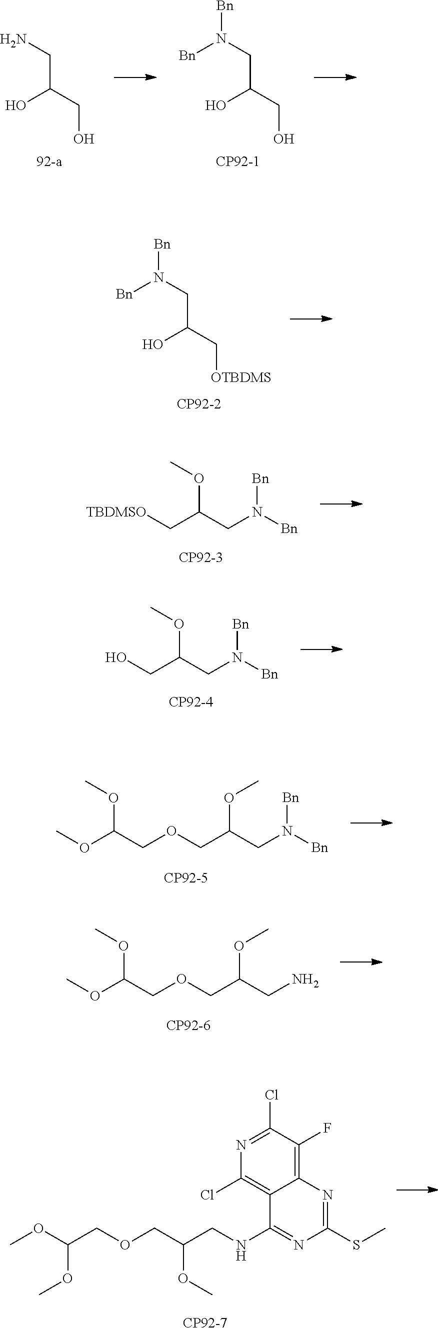

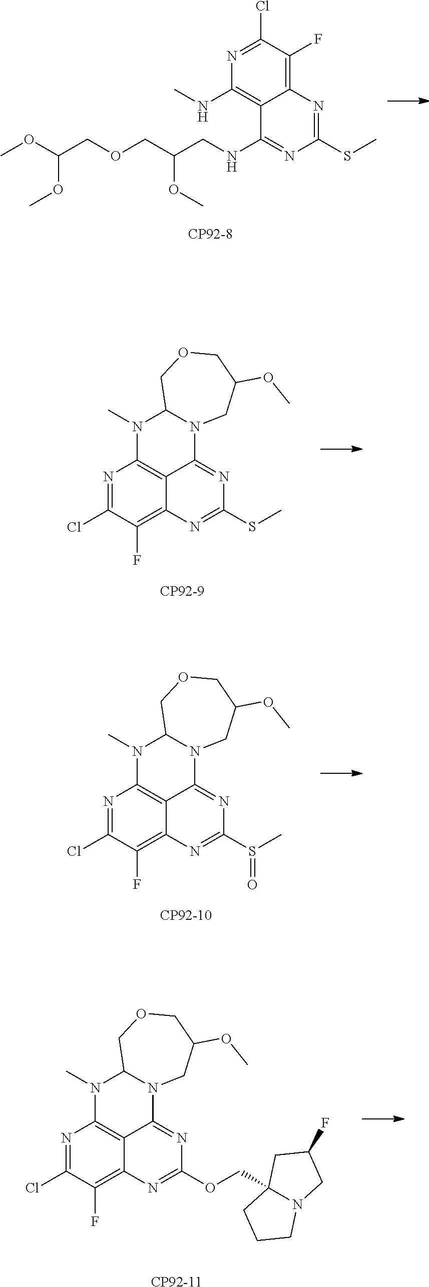

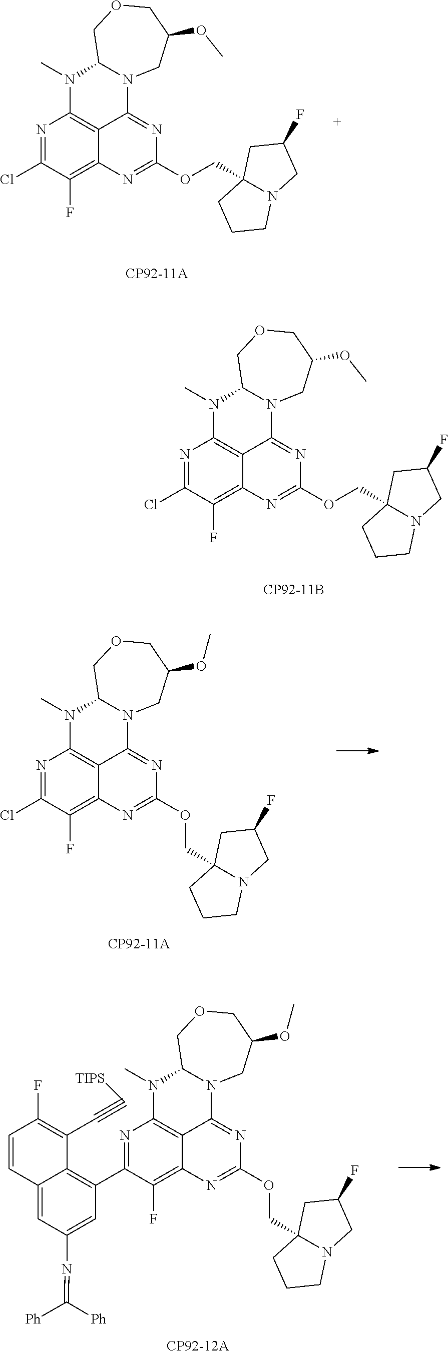

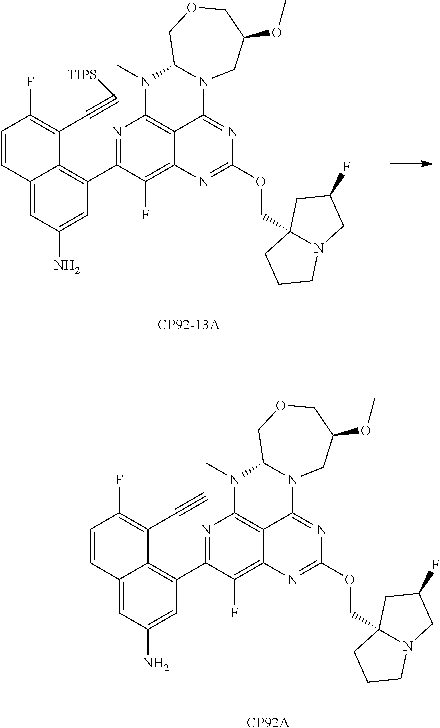

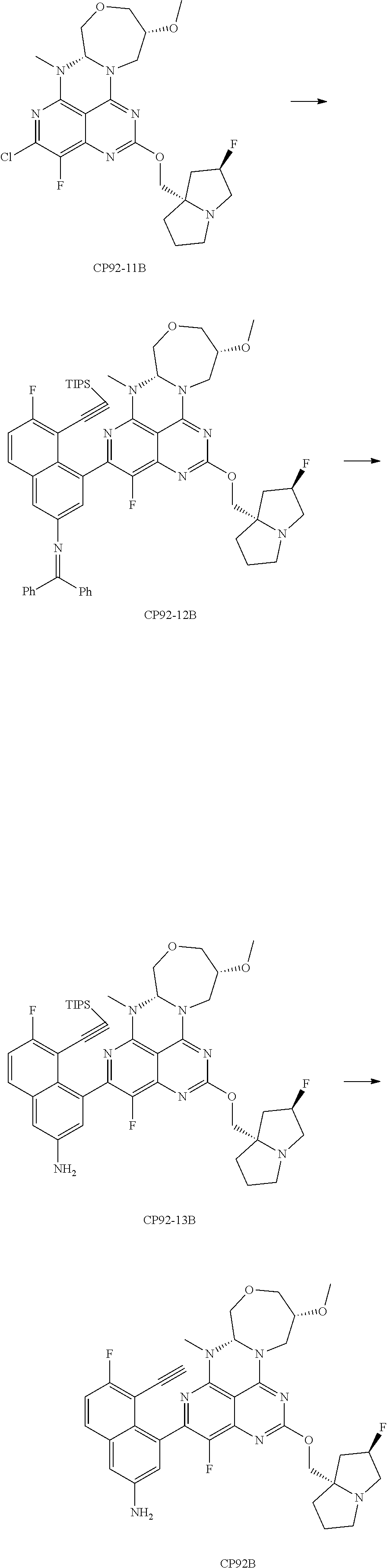

Benzyl bromide (57.81 g) was dropped into a solution of 92-a (9.9 g), K₂CO₃ (47.17 g) in ethanol (300 mL) at 100° C. The solution was stirred at 100° C. for 15 h under N₂. The mixture was concentrated, diluted with NaHCO₃ solution, extracted with EA and post processed to give CP92-1 (20.61 g). MS: m/z: 272[M+H]⁺.

A solution of CP92-1 (20.61 g), DMAP (9.79 g), TEA (12.12 g) and TBDMSCl (13.82 g) in DCM (200 mL) was stirred at RT for 20 h. The solution was diluted with NaHCO₃ solution and post processed to give CP92-2 (23.24 g). MS: m/z: 386[M+H]⁺.

NaH (642 mg) was added into a solution of CP92-2 (4.15 g) in THF (50 mL) at 0° C. The solution was stirred at 0° C. for 10 min under N₂. Methyl iodide (3.27 g) was dropped into the mixture. The solution was stirred at RT for 16 h under N₂, quenched with water, extracted with EA and post processed to give CP92-3 (4.286 g). MS: m/z: 400[M+H]⁺.

A solution of CP92-3 (4.286 g), CsF (6.240 g) in DMF (40 mL) was stirred at 35° C. for 16 h. The solution was diluted with EA, washed with NaCl solution and post processed to give CP92-4 (2.518 g). MS: m/z: 286[M+H]⁺.

CP92-11 was synthesized in a manner similar to CP46-10 with CP92-4.

CP92-11 was separated by Prep-HPLC-Gilson with the conditions: Column, CHIRAL ART Cellulose-SC, 20 mm×250 mm, Sum; mobile phase (Hex:DCM=3:1)(0.1% DEA)/EtOH (50:50); Flowing rate: 15 mL/min, 220 nm. This resulted in CP92-12A (58 mg, the first eluting isomer, Retention time: 6.797 min) and CP92-12B (26 mg, the second eluting isomer, Retention time: 8.2 min). MS: m/z: 497[M+1]⁺.

Compound 92A (CP92A, 36.8 mg, assumed) was synthesized by the similar procedure of the synthesis of CP46 with CP92-12A. MS: m/z: 646[M+1]⁺.

Compound 92B (CP92B, assumed) was synthesized by the similar procedure of the synthesis of CP46 with CP92-12B. MS: m/z: 646[M+1]⁺.

### Example 95

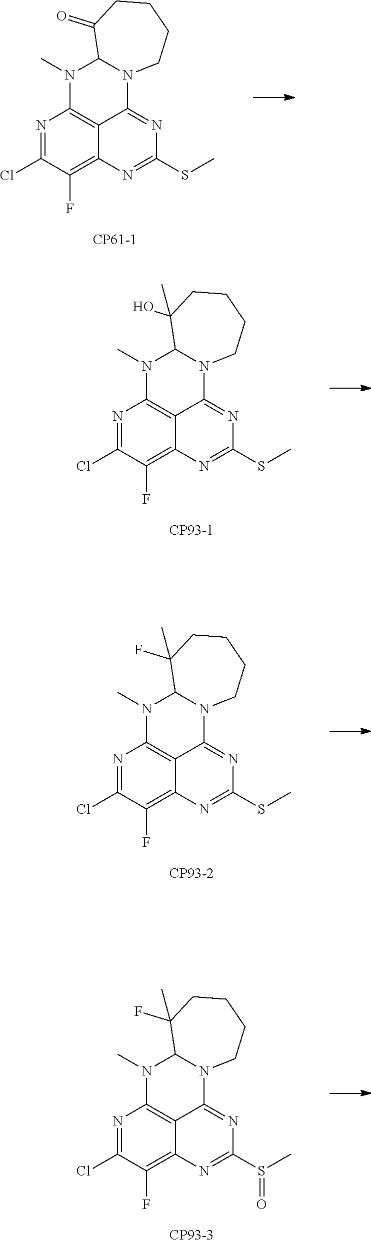

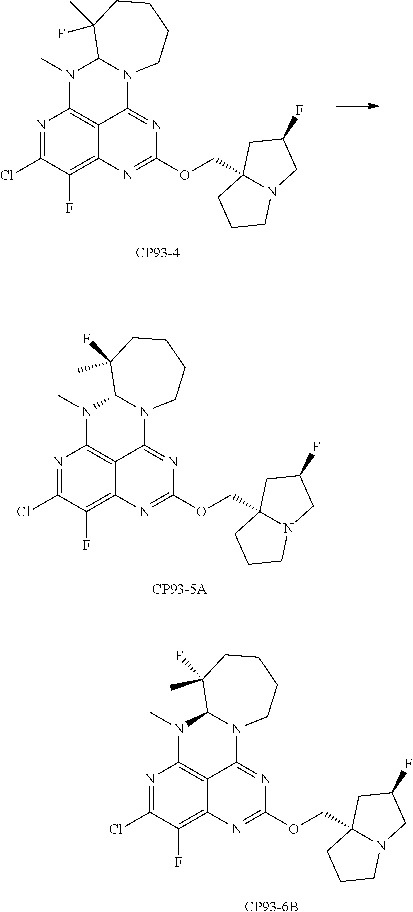 [SMILES: CN1c2nc(Cl)c(F)c3nc(OC[C@@]45CCCN4C[C@H](F)C5)nc(c23)N2CCCCC(C)(F)C12 ; "CP93-4" ; CN1c2nc(Cl)c(F)c3nc(OC[C@@]45CCCN4C[C@H](F)C5)nc(c23)N2CCCC[C@](C)(F)[C@@H]12 ; "CP93-5A" ; CN1c2nc(Cl)c(F)c3nc(OC[C@@]45CCCN4C[C@H](F)C5)nc(c23)N2CCCC[C@@](C)(F)[C@H]12 ; "CP93-6B"]

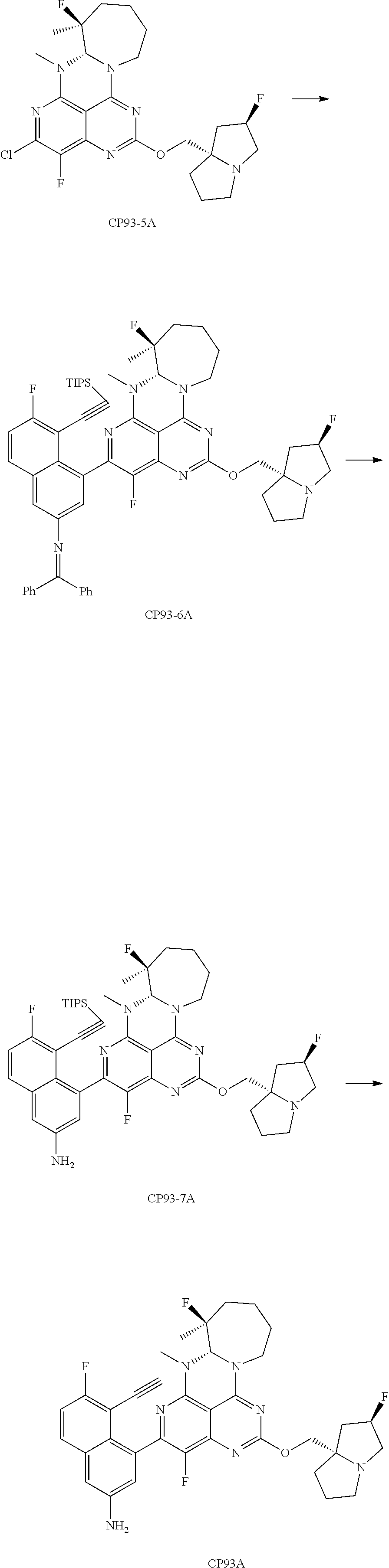

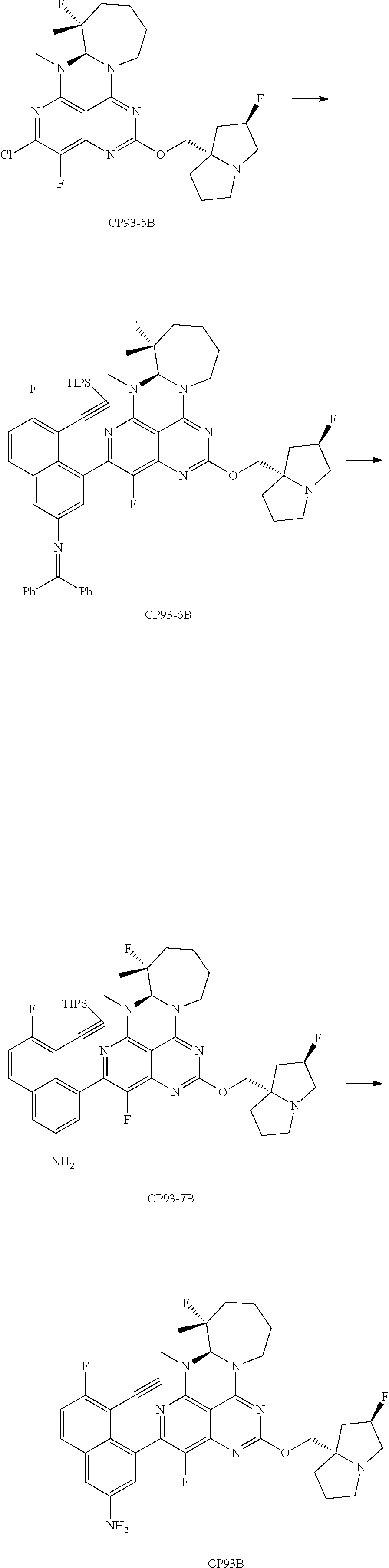

Methylmagnesium bromide (8.5 mL) was dropped into a solution of CP61-1 (1.58 g) in THF (150 mL) at 0° C. The solution was stirred at 45° C. for 36 h under N₂, quenched with NH₄Cl solution, extracted with EA and post processed to give CP93-1 (674 mg). MS: m/z: 384[M+H]⁺.

A solution of CP93-1 (674 mg), DAST (0.99 g) in DCM (10 mL) was stirred at 45° C. for 3 h under N₂. The mixture was cooled to RT, diluted with NaHCO₃ solution and post processed to give CP93-2 (276 mg). MS: m/z: 386[M+H]⁺.

CP93-4 was synthesized in a manner similar to CP46-10 with CP93-2.

CP93-4 was separated by Prep-HPLC-Gilson with the conditions: CHIRAL ART Cellulose-SC, 20 mm×250 mm, Sum; mobile phase, (Hex:DCM=3:1)(0.1% DEA)/IPA (50:50); Flowing rate: 15 mL/min, 220 nm. This resulted in CP93-5A (50 mg, the first eluting isomer, Retention time: 4.933 min) and CP93-5B (49 mg, the second eluting isomer, Retention time: 7.847 min). MS: m/z: 497[M+1]⁺.

Compound 93A (CP93A, 32.4 mg, confirmed) was synthesized by the similar procedure of the synthesis of CP46 with CP93-5A. MS: m/z: 646[M+1]⁺.

Compound 93B (CP93B, 32.7 mg, confirmed) was synthesized by the similar procedure of the synthesis of CP46 with CP93-5B. MS: m/z: 646[M+1]⁺.

### Example 96

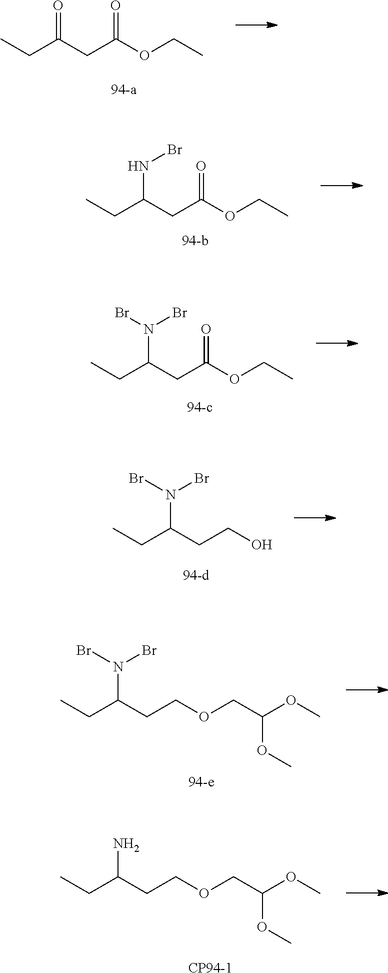

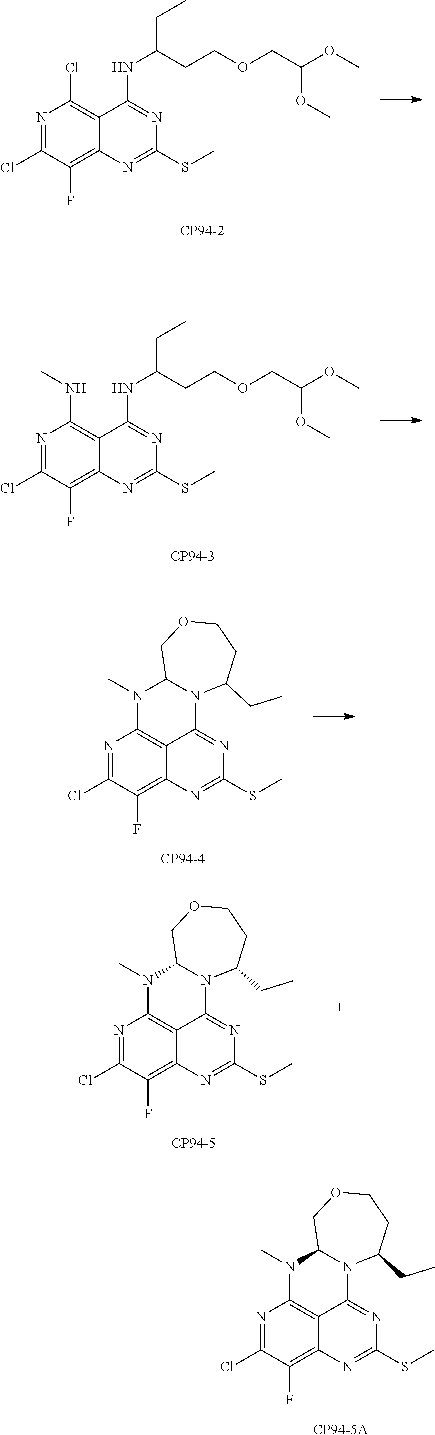

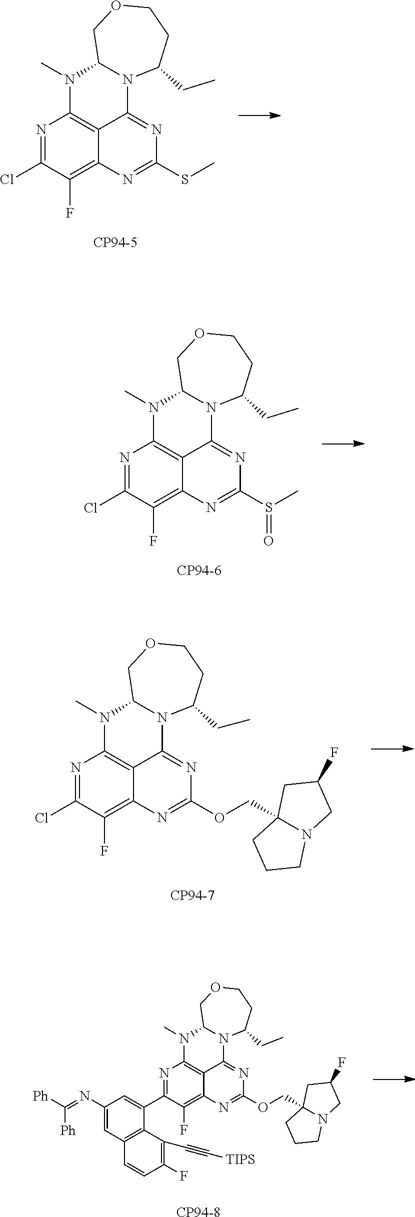

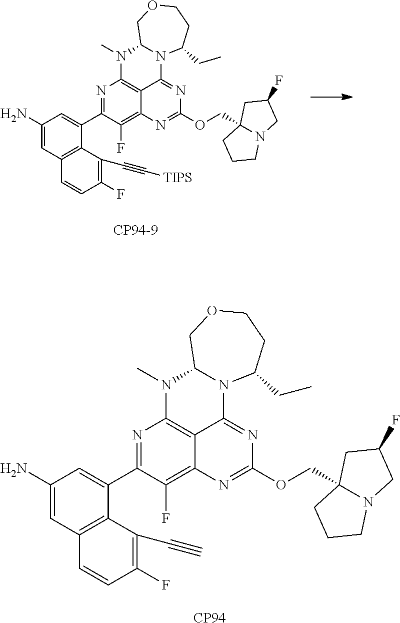

To a solution of 94-a (10.09 g) in DCE (150 mL), benzylamine (22.87 g), triethylamine (28.76 g) was added titanium tetrachloride (55.12 g) in DCM (50 mL) and the resulting mixture was stirred at 0° C. and stirred at RT for Sb. Sodium cyanoboronhydride (6.78 g) in MeOH (15 mL) was added at 0° C. The solution was stirred for 16 h at RT. The mixture was adjusted to pH 8 with NaHCO₃(aq). The mixture was diluted with EA and post processed to afford 94-b (4.86 g). MS: m/z: 236[M+H]⁺.

To a solution of 94-b (4.50 g) in ACN (10 mL) were added K₂CO₃ (7.95 g), TBAI (0.85 g) and BnBr (6.69 g) and the resulting mixture was stirred at RT for 16 h. The solution was filtered and diluted with EA. The mixture was concentrated and purified to afford 94-c (4.86 g). MS: m/z: 326[M+H]⁺.

A solution of 94-c (4.95 g) in THF (50 mL) was added LAH (1.22 g) at 0° C. The resulting mixture was stirred at 25° C. for 2 h. The mixture was diluted with Na₂SO₄·10H₂O. The solution was filtered and diluted with EA. The mixture was concentrated and purified to give 94-d (4.48 g). MS: m/z: 284[M+H]⁺.

To a solution of 94-d (4.48 g) in THF (50 mL) were added NaH (1.61 g), DMAP (0.10 g) and TBAI (0.35 g) at 0° C. The resulting mixture was stirred at RT for 5 h at 70° C., diluted with EA and post processed to give 94-e (5.01 g). MS: nm/z: 372[M+H]⁺.

To a solution of 94-a (2.06 g) in methanol (20 mL) were added Pd(OH)₂/C (0.44 g) and Pd/C (0.43 g) at H₂. The reaction mixture was stirred at 25° C. for 16 hours, filtered and the filtrate was concentrated to afford CP94-1 (0.97 g, crude). MS: m/z: 192[M+H]⁺.

CP94-4 was synthesized in a manner similar to CP46-8 with CP94-1.

CP94-4 was separated by Prep-HPLC-Gilson with the conditions: Column, CHIRALART Cellulose-SC, 20 mm×250 mm, 5 um; mobile phase. (Hex:DCM=3:1)(0.1% DEA)/IPA (50:50); Flowing rate: 17 mL/min, 220 nm). This resulted in CP94-5A (the first eluting isomer, Retention time 4.728 min) and CP94-5 (the second eluting isomer, Retention Time 10.189 min). MS: m/z: 384[M+H]⁺.

Compound 94 (CP94, confirmed) was synthesized the procedure of CP46 with CP94-5. LCMS: m/z: 644[M+H]⁺.

### Example 97

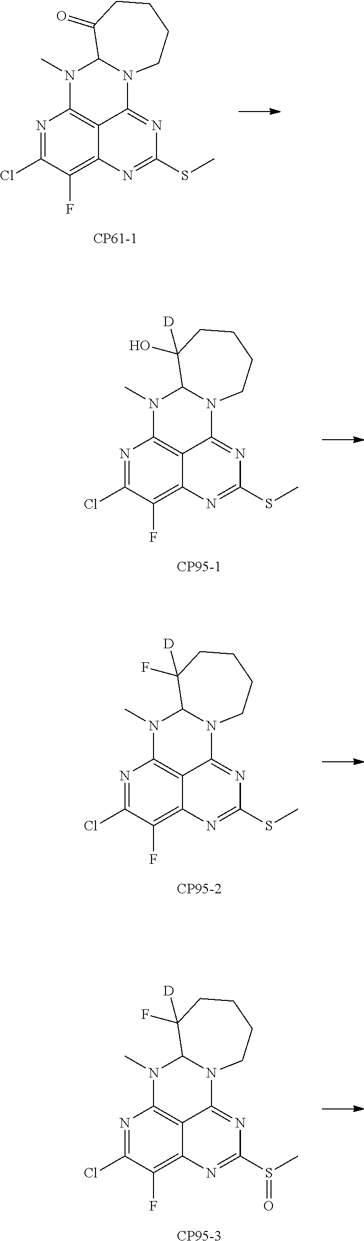

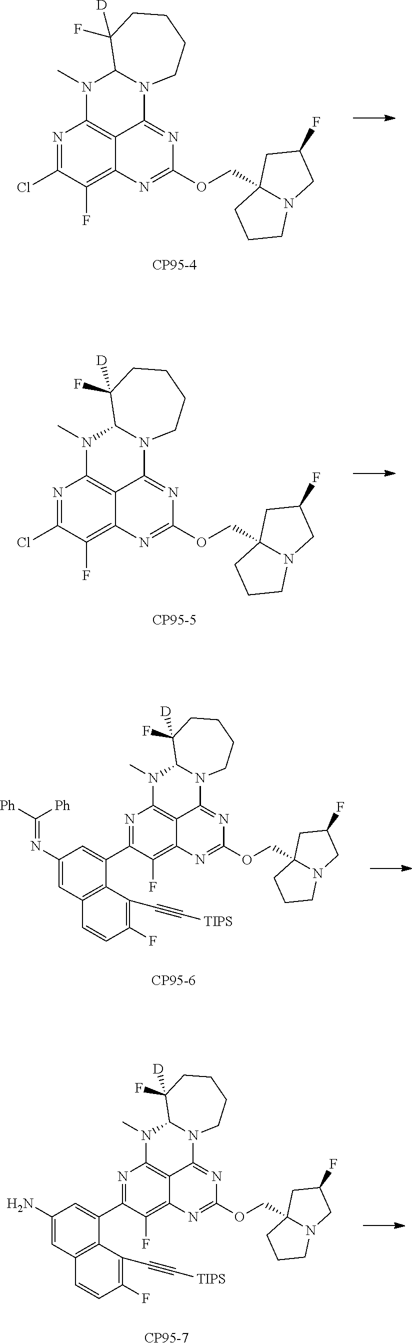

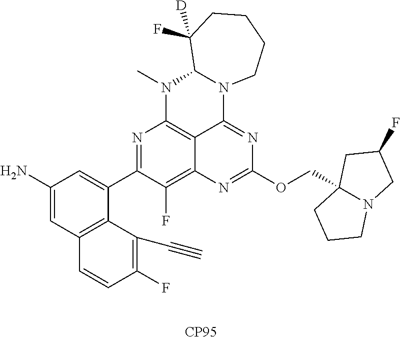 [SMILES: "CP95" ; [2H][C@]1(F)CCCCN2c3nc(OC[C@@]45CCCN4C[C@H](F)C5)nc4c(F)c(-c5cc(N)cc6ccc(F)c(C#C)c56)nc(c34)N(C)[C@@H]21]

To a solution of CP61-1 (2.16 g) in DCM (150 mL) and THF (150 mL) was added NaBD₄ (0.75 g) and the resulting mixture was stirred 16 h at RT, quenched with water, extracted with DCM and post processed to afford CP95-1 (1.93 g, crude). MS: m/z: 371 [M+H]⁺.

CP95-4 was synthesized in a manner similar to CP40A-4 with CP95-1

CP95-4 was separated by Prep-HPLC-Gilson with the conditions: CHIRAL ART Cellulose-SC, 20 mm×250 mm, 5 μm; mobile phase, (Hex:DCM=3:1) (0.1% DEA)/IPA (50:50); Flowing rate: 15 mL/min. CP95-5 (the second eluting isomer, Retention time: 7.580 min).

Compound 95 (CP95, confirmed) was synthesized the procedure of CP93. LCMS: m/z: 633 [M+H]⁺.

### Example 98

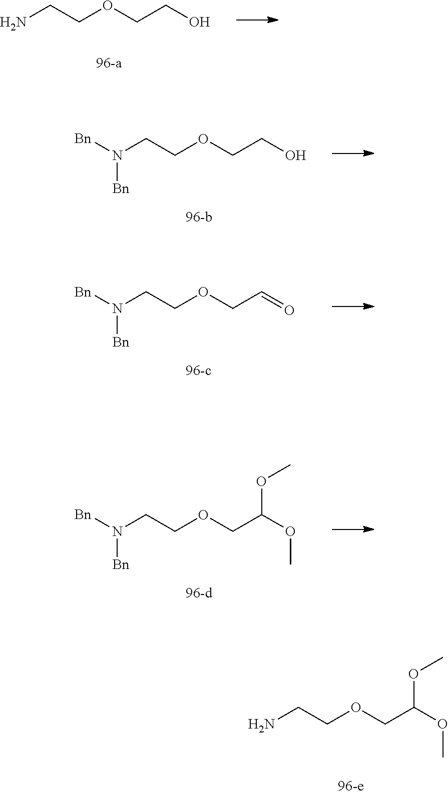

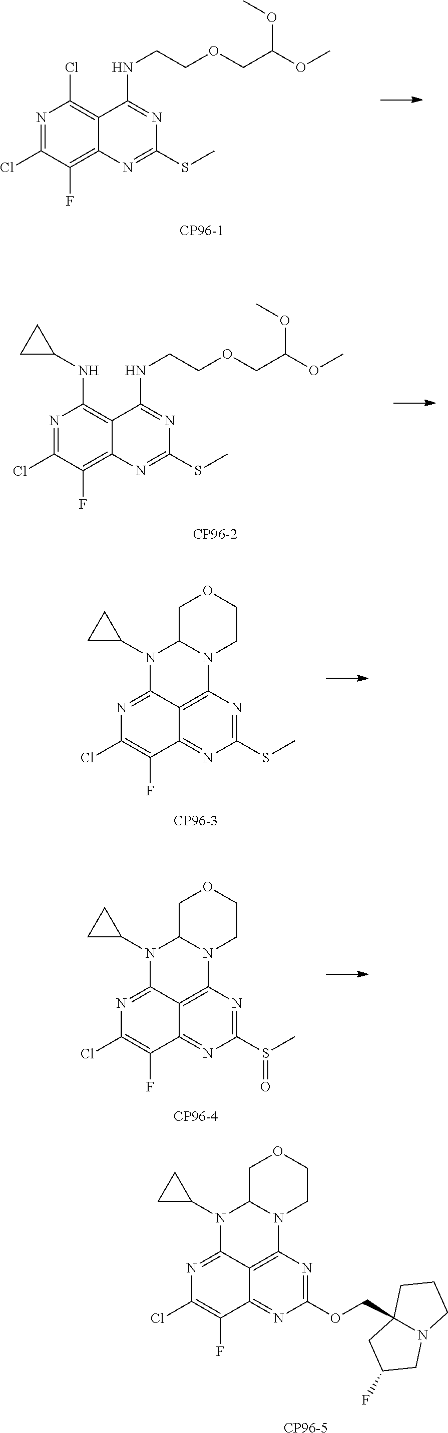

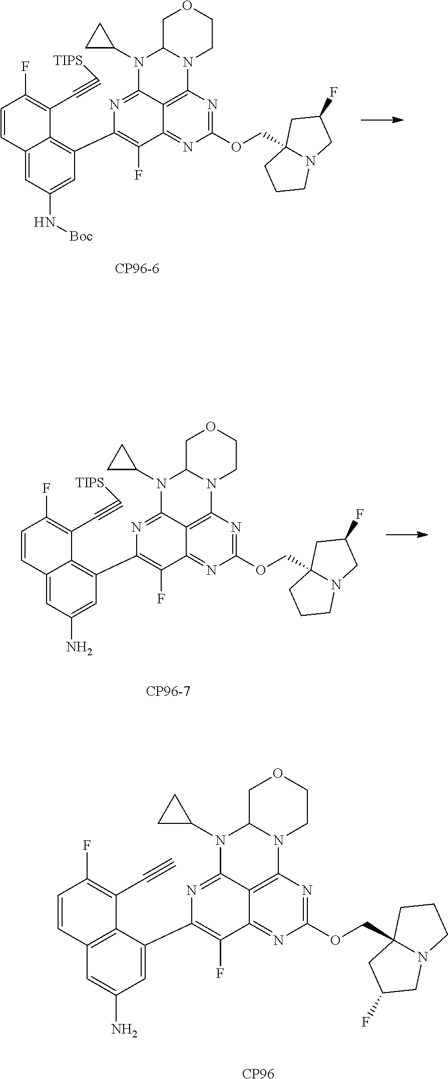

A solution of 96-a (5.07 g), benzyl bromide (20.93 g), K₂CO₃ (16.70 g) and TBAI (10.5 g) in MeCN (100 mL) was stirred at RT overnight. The reaction solution was filtered, washed with EA, concentrated and purified to give 96-b (1.38 g). MS: m/z: 286[M+H]⁺.

To a solution of 96-b (6.32 g) and DCM (25 mL) in DMSO (50 mL) was added triethylamine (19.06 g) and pyridine sulfur trioxide (14.23 g) and the resulting mixture was stirred at 0° C. for 3 h, quenched with Na₂S₂O₃, extracted with EA and post processed to give 96-c (2.78 g). MS: m/z: 284[M+H]⁺.

A solution of 96-c (3.85 g), p-TsOH·H₂O (2.65 g) and methyl orthoformate (4.36 g) in toluene (40 mL) was stirred at 100° C. for 16 hours under N₂. The mixture was allowed to cool to RT, diluted with EA and post processed to give 96-d (2.35 g). MS: m/z: 330[M+H]⁺.

To a solution of 96-d (2.35 g) in methanol (20 ml) was added Pd/C (0.43 g) at H₂. The reaction mixture was stirred at 25° C. for 16 hours. The resulting mixture was filtered and the filtrate was concentrated to afford 96-e (873 mg). MS: m/z: 150[M+H]⁺.

Compound 96 (CP96, confirmed) was synthesized the procedure of CP46 with CP96-e. LCMS: m/z: 628[M+H]⁺.

### Example 99

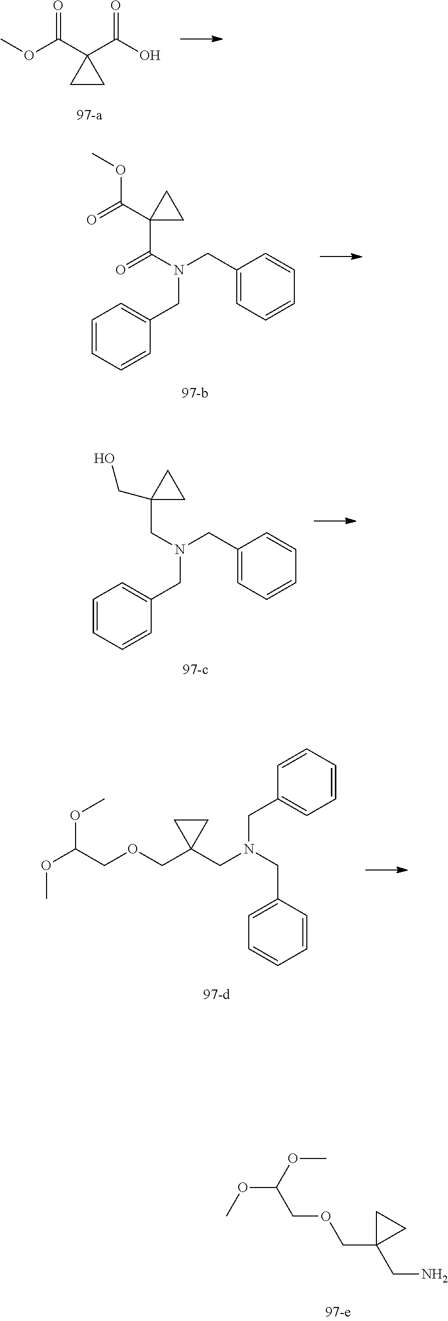

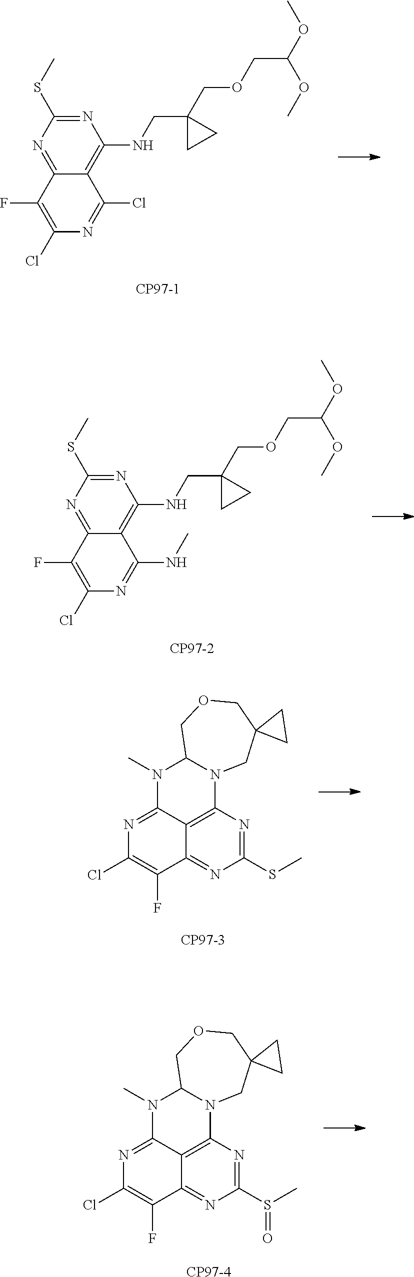

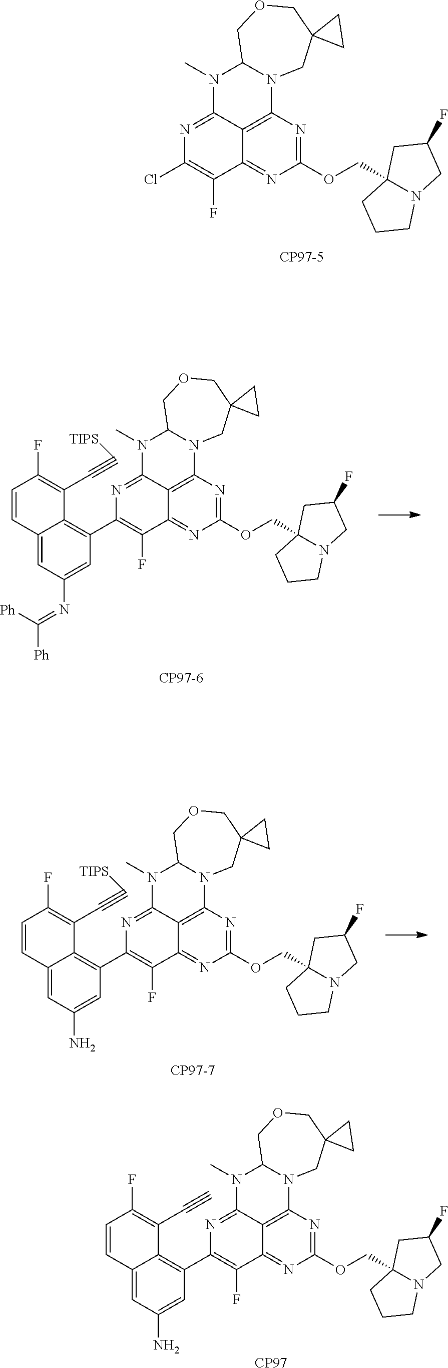

A solution of 97-a (10.12 g), dibenzylamine (13.72 g), DMAP (1.82 g) and EDCI (23.62 g) in DCM (200 mL) was stirred at RT overnight, quenched with NH₄Cl, extracted with DCM, concentrated and purified to give 97-b (14.09 g). MS: m/z: 324[M+H]⁺.

A solution of 97-b (14.09 g), BH₃/THF (120 mL) in THE (80 mL) was stirred at 60° C. for 16 hours under N₂. The reaction mixture was added MeOH (120 mL), stirred at 60° C. for 30 min and concentrated. The mixture was added THF (80 mL) and LAH (1.29 g) at 0° C. and stirred at room for 2 h, quenched with Na₂SO₄ decahydrate and post processed to give 97-c (9.44 g). MS: m/z: 282[M+H]⁺.

Compound 97 (CP97, confirmed) was synthesized the procedure of CP46 with 97-c. LCMS: m/z: 642[M+H]⁺.

### Example 100

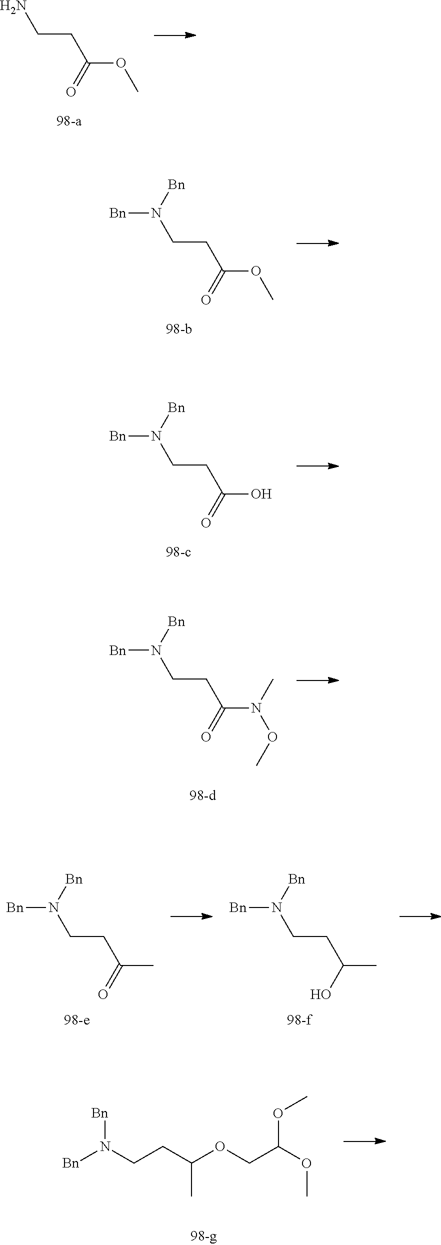

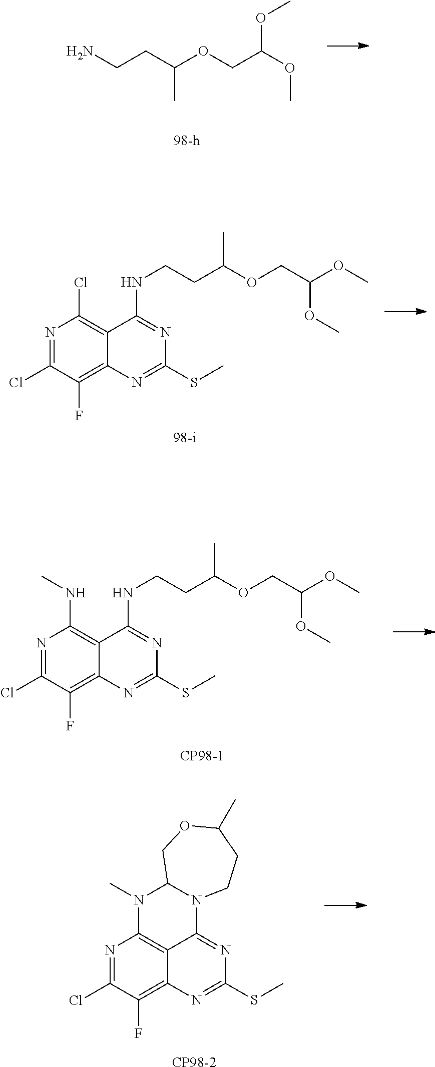

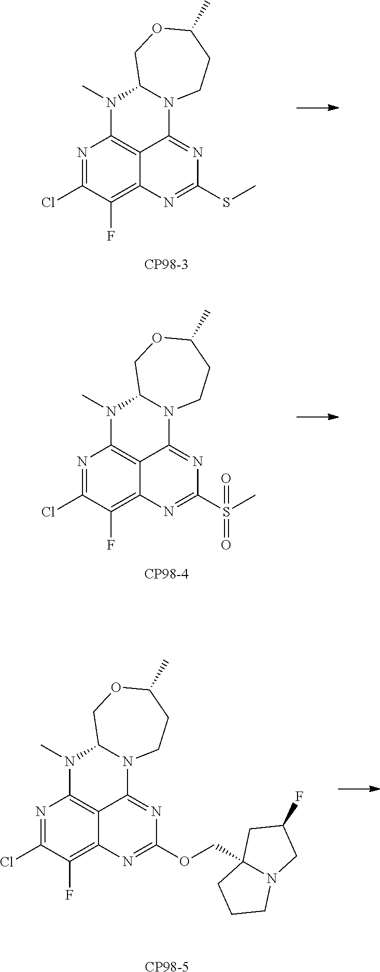 [SMILES: "CP98-3" ; "CP98-4" ; "CP98-5" ; C[C@@H]1CCN2c3nc(OC[C@@]45CCCN4C[C@H](F)C5)nc4c(F)c(Cl)nc(c34)N(C)[C@@H]2CO1 ; CSc1nc2c3c(nc(Cl)c(F)c3n1)N(C)[C@@H]1CO[C@H](C)CCN21 ; C[C@@H]1CCN2c3nc(S(C)(=O)=O)nc4c(F)c(Cl)nc(c34)N(C)[C@@H]2CO1]

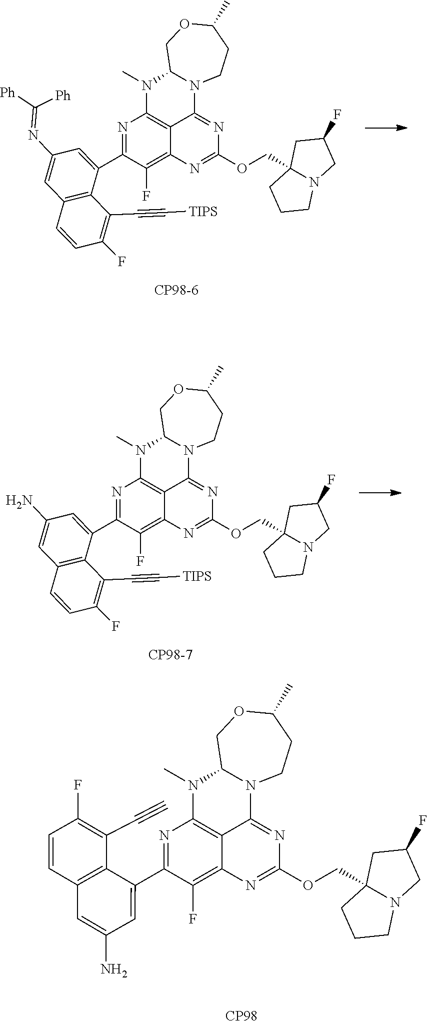

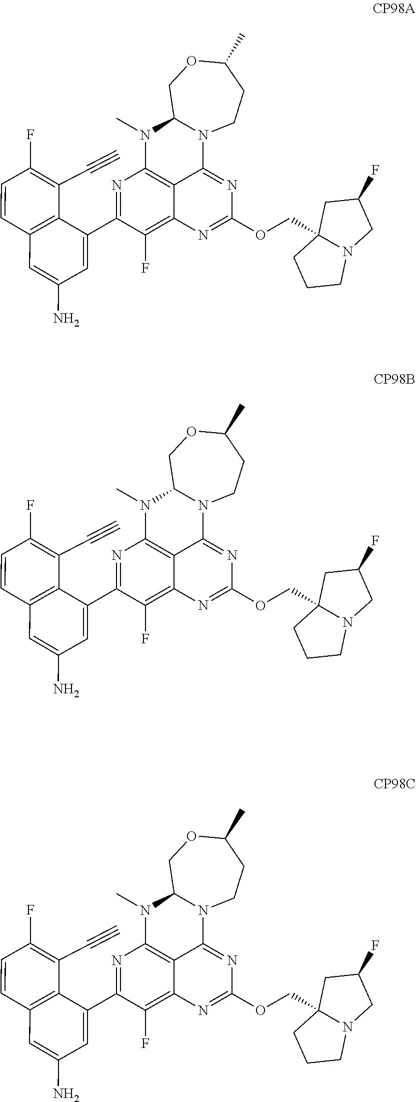 [SMILES: C#Cc1c(F)ccc2cc(N)cc(-c3nc4c5c(nc(OC[C@@]67CCCN6C[C@H](F)C7)nc5c3F)N3CC[C@@H](C)OC[C@@H]3N4C)c12 ; C#Cc1c(F)ccc2cc(N)cc(-c3nc4c5c(nc(OC[C@@]67CCCN6C[C@H](F)C7)nc5c3F)N3CC[C@H](C)OC[C@H]3N4C)c12 ; C#Cc1c(F)ccc2cc(N)cc(-c3nc4c5c(nc(OC[C@@]67CCCN6C[C@H](F)C7)nc5c3F)N3CC[C@H](C)OC[C@@H]3N4C)c12]

A solution of 98-a hydrochloride (10.32 g), TBAI (1.41 g), K₂CO₃ (33.18 g) and BrBn (32.05 g) in MeCN (100 mL) was stirred at RT for 16 h. The mixture was concentrated and purified to afford 98-b (17.2 g). MS: m/z: 284[M+H]⁺.

To a solution of 98-b (17.2 g) in MeOH (40 mL) and H₂O (40 mL) was added NaOH (3.23 g). The reaction mixture was stirred at RT for 16 h, adjusted to pH-3, concentrated, quenched with water, extracted with EA and post processed to afford 98-c (12.3 g). MS: m/z: 270[M+H]⁺.

To a solution of 98-c (12.3 g) in DMF (50 mL) was added N,O-dimethylhydroxylamine hydrochloride (5.96 g), HATU (20.85 g) and DIPEA (20.54 g). The reaction mixture was stirred at RT for 2 h. Upon completion, water was added and the aqueous layer was extracted with EA and post processed to afford 98-d (13.95 g). MS: m/z: 313[M+H]⁺.

To a solution of 98-d (13.95 g) in THF (150 mL) was added MeMgBr (24.5 mL) at 0° C. The mixture was stirred at RT for 8 h, quenched with aq. NH₄C1 and H₂O was added. The resulting mixture was extracted with DCM and post processed to afford 98-e (5.8 g). MS: m/z: 268[M+H]⁺.

To a solution of 98-e (5.8 g) in MeOH (50 mL) was added NaBH₄ (1.24 g). The reaction mixture was stirred at RT for 1 h, concentrated, quenched with water, extracted with EA and post processed to afford 98-f (5.75 g). MS: m/z: 270[M+H]⁺.

CP98-2 was synthesized the procedure of CP46-8 with CP98-f.

CP98-2 (3.12 g) was separated by Prep-HPLC-Gilson with the conditions: Column, CHIRAL ART Cellulose-SC, 20 mm×250 mm, 5 um; mobile phase, (Hex:DCM=3:1)(0.1% DEA)/EtOH (50:50); Flowing rate: 15 mL/min. CP98-3 (240 mg, the third eluting isomer, Retention time: 9.080 min) was obtained.

Compound 98 (CP98) was synthesized the procedure of CP46 with CP98-3.

Compound 98A (CP98A), Compound 98B (CP98B), Compound 98C (CP98C) were synthesized in a similar method. LCMS: m/z: 630[M+H]⁺.

### Example 101

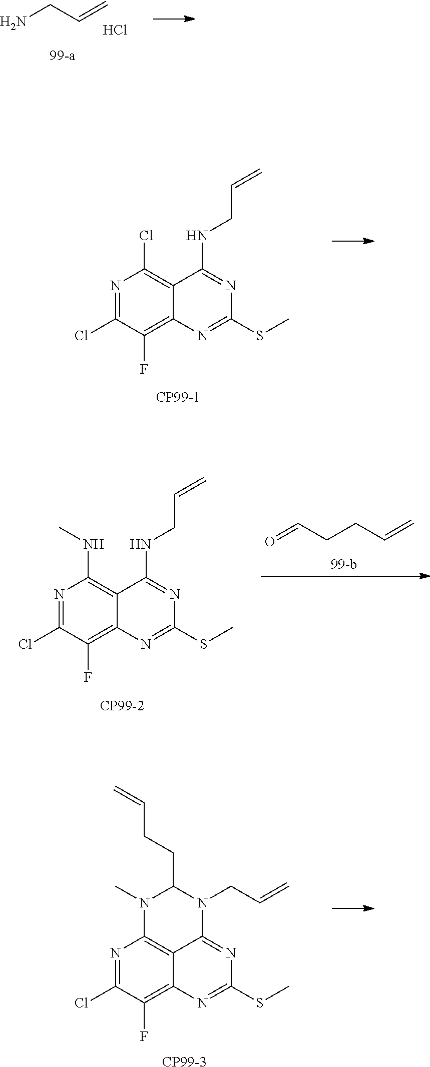

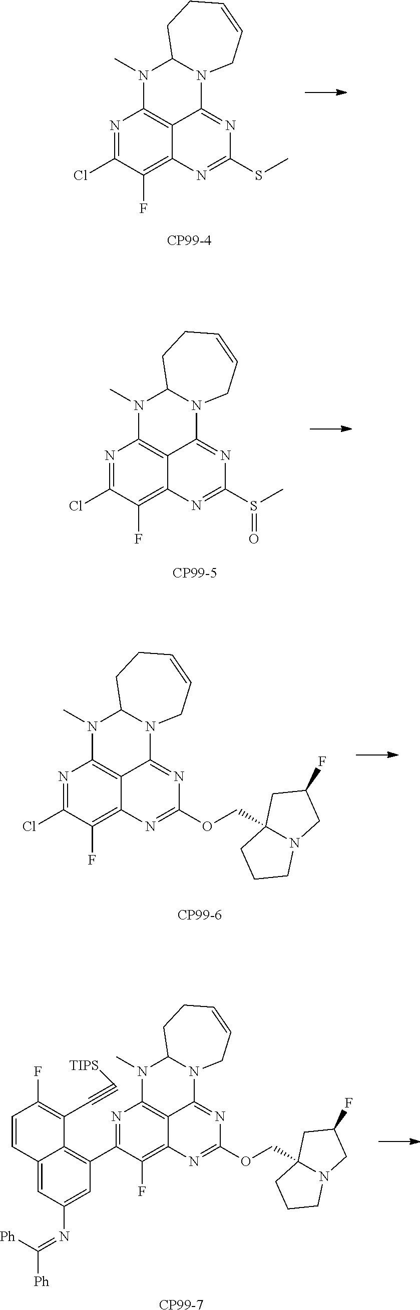

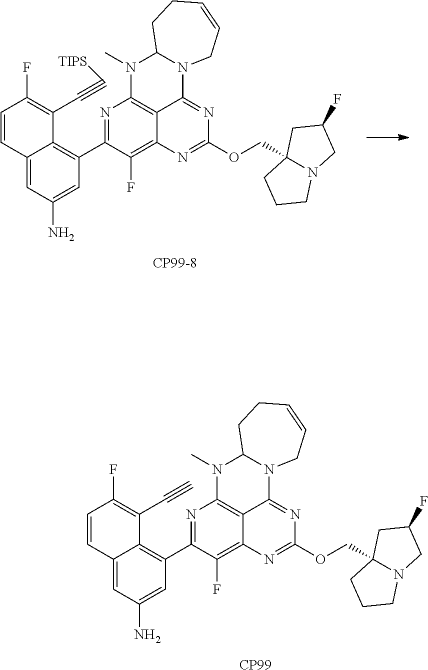

Compound 99 (CP99, confirmed) was synthesized the similar procedure of CP71. LCMS: m/z: 612[M+H]⁺.

### Example 102

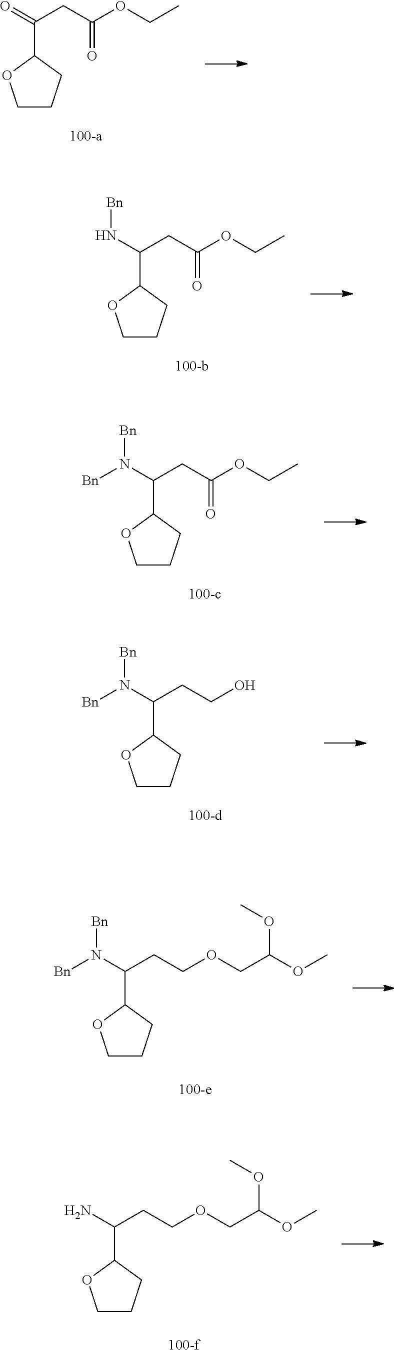

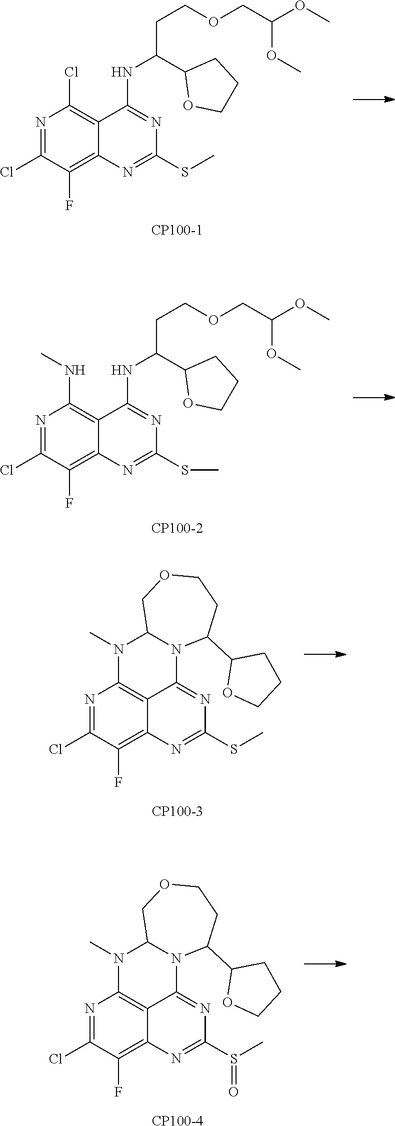

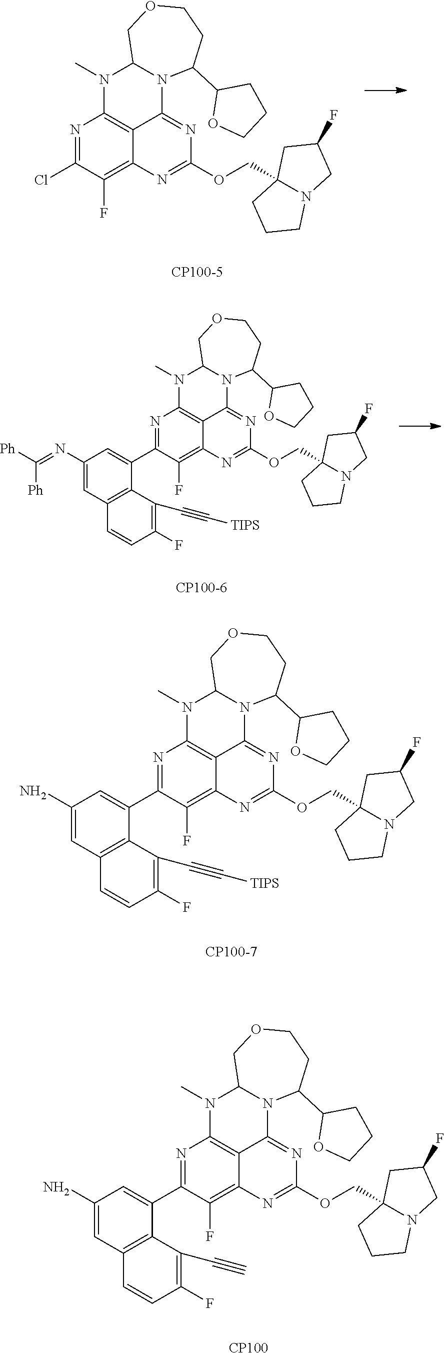

Compound 100 (CP100, confirmed) was synthesized the similar procedures of synthesis of CP94 with CP100-d. LCMS: m/z: 686[M+H]⁺.

### Example 103

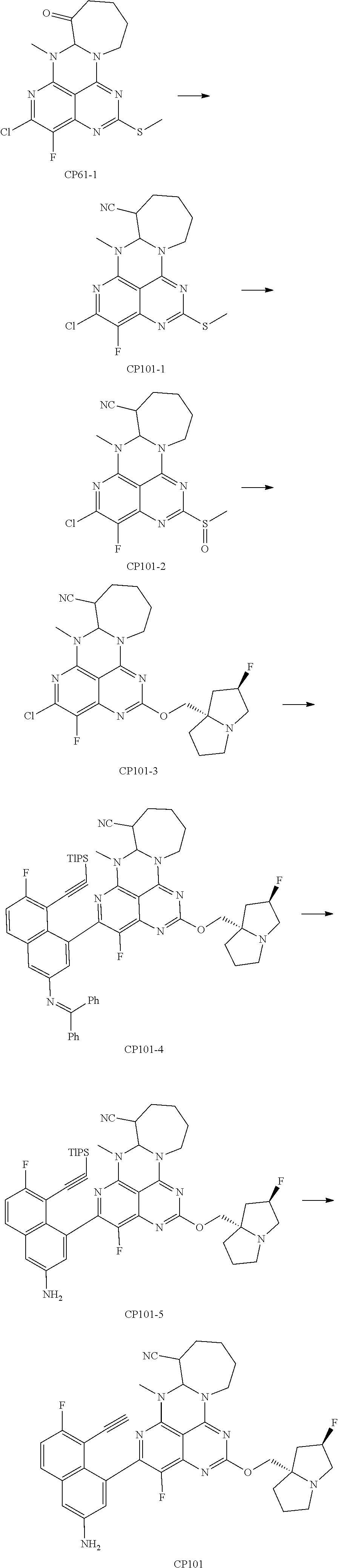

To a solution of CP61-1 (1.139 g) and tosylmethyl isocyanide (0.876 g) in dioxane (40 mL) was added potassium tert-butoxide (0.415 g) at 0° C. The mixture was stirred at 50° C. for 0.5h, and stirred at RT for 16 h. The reaction was diluted with EA and post processed to afford CP101-1 (587 mg). MS: m/z: 379[M+H]⁺.

Compound 101 (CP101, confirmed) was synthesized the similar procedure of preparation of CP46 with CP101-1. LCMS: m/z: 639[M+H]⁺.

### Example 104

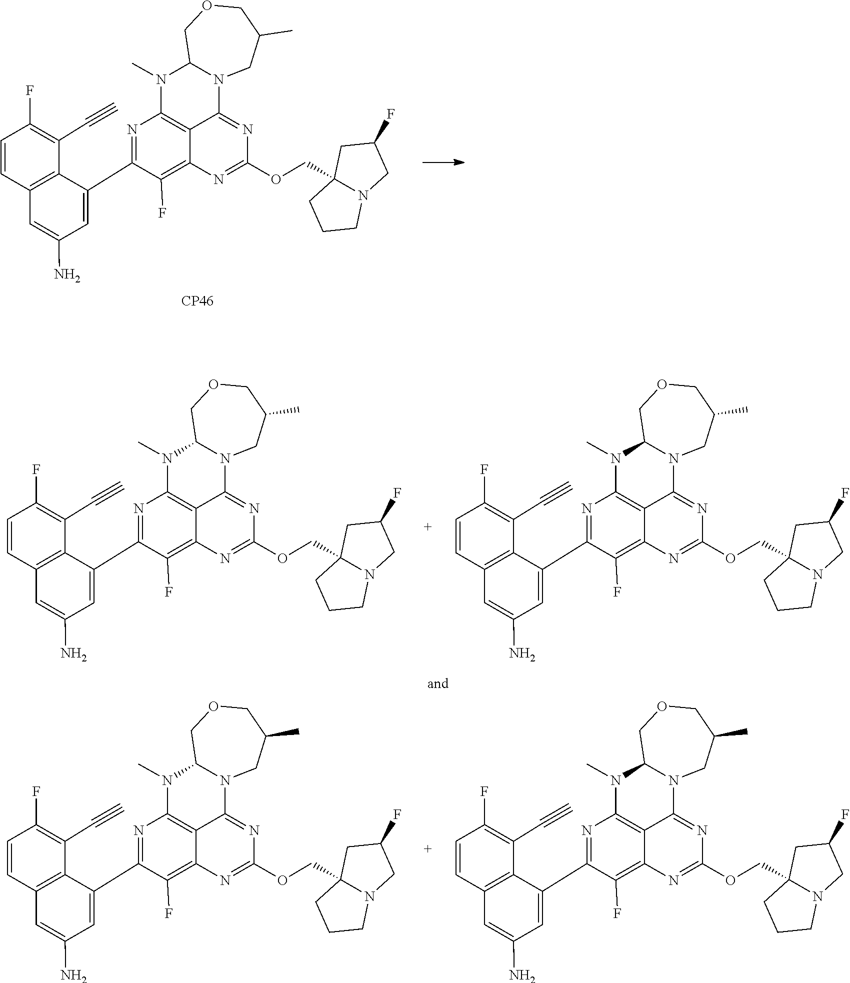

CP46 was separated by Prep-HPLC-Gilson with the conditions: Column, CHIRAL ART Cellulose-SA column (2 cm×25 cm, 5 um); mobile phase, Hex (0.1% DEA)/EtOH (50:50); Flowing rate: 20 mL/min. This resulted in Compound 102 (CP102, 17.5 mg, the first eluting isomer. Retention time 7.034 min) and Compound 103 (CP103, 5.7 mg, the second eluting isomer, Retention Time 10.862 min). MS: m/z: 630[M+H]⁺.

### Example 105

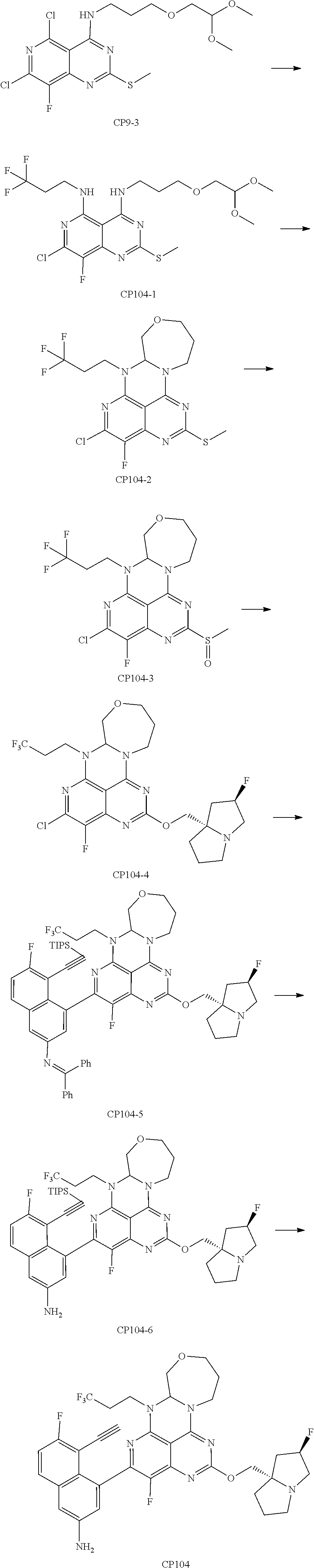

A mixture of CP9-3 (0.81 g), 3,3,3-trifluoropropan-1-amine hydrochloride (0.85 g), DIEA (1.19 g) and DMA (10 mL) in 48 mL sealed bottle was stirred at 70° C. for 17 h. The mixture was diluted with water and extracted with EA and post processed to afford CP104-1 (0.02 g). MS: m/z: 502[M+H]⁺.

Compound 104 (CP104, confirmed) was synthesized the similar procedures of the preparation of CP46 with CP104-1. LCMS: m/z: 698[M+H]⁺.

### Example 106

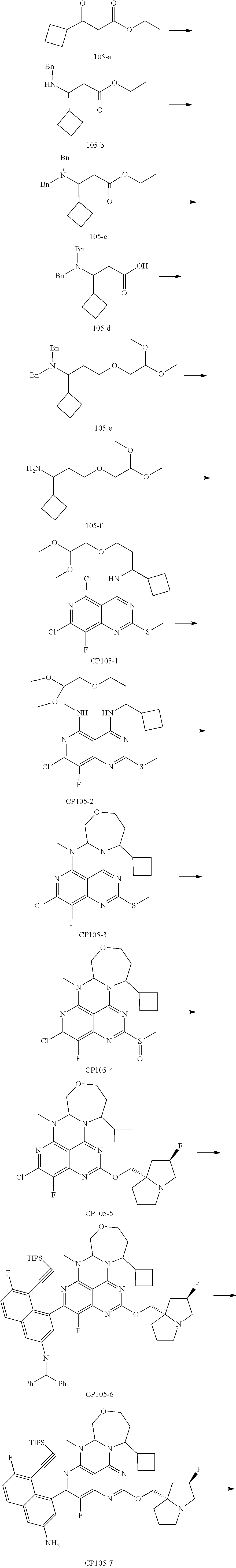

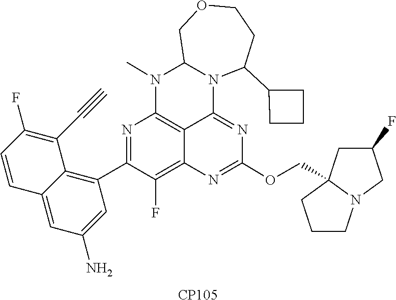 [SMILES: "CP105" ; C#Cc1c(F)ccc2cc(N)cc(-c3nc4c5c(nc(OC[C@@]67CCCN6C[C@H](F)C7)nc5c3F)N3C(C5CCC5)CCOCC3N4C)c12]

Compound 105 (CP105, confirmed) was synthesized the similar procedures of the preparation of CP94 with CP105-a. LCMS: m/z: 670[M+H]⁺.

### Example 107

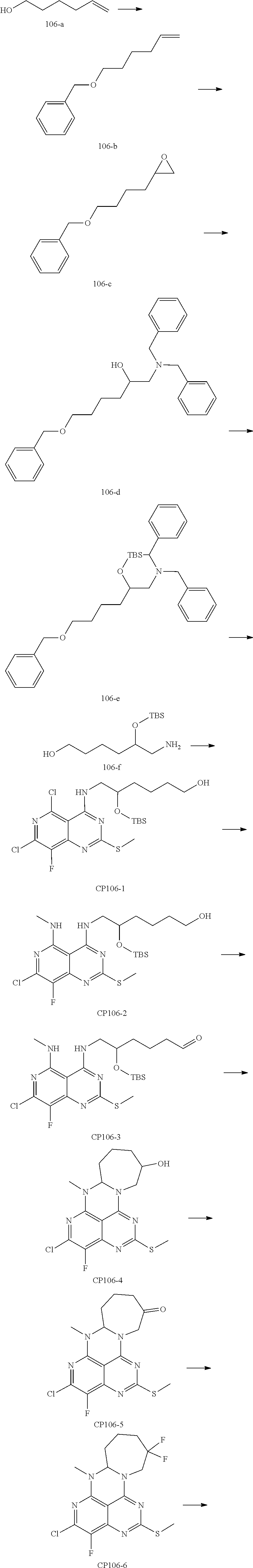

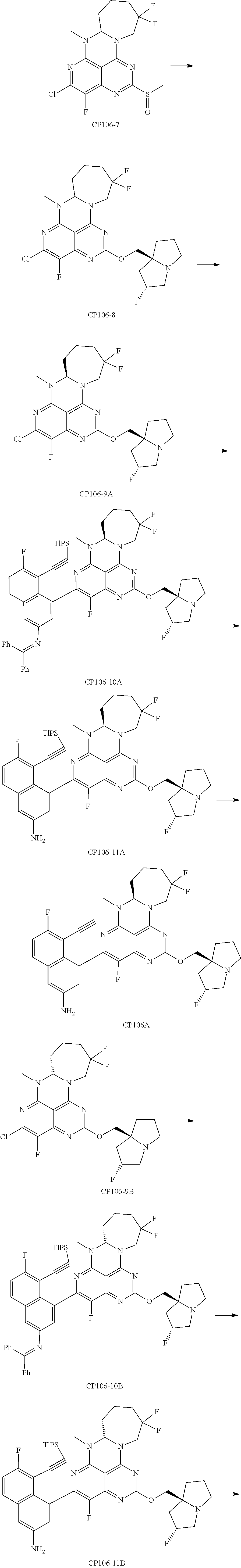

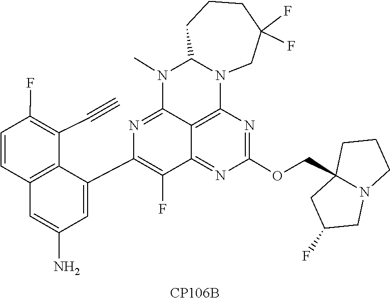

To a solution of 106-a (19.93 g) in THF (200 mL) was added NaH (15.96 g) at 0° C. and the resulting mixture was stirred at room for 1 h. The reaction mixture was added bromoacetaldehyde dimethyl acetal (6.29 g), benzyl bromide (35.67 g) and N-(4-pyridyl)dimethylamine (2.07 g) and the resulting mixture was stirred at 25° C. for 16 hours under N₂, quenched with water, extracted with EA and post processed to give 106-b (34.24 g). MS: m/z: 191[M+H]⁺.

A solution of 106-b (34.24 g), m-chloroperoxybenzoic acid (40.60 g) in DCM (300 mL) was stirred at 25° C. for 16 hours, quenched with Na₂S₂O₃, extracted with DCM and post processed to give 106-c (20.94 g). MS: m/z: 207[M+H]⁺.

A solution of 106-c (18.48 g) and dibenzylamine (53.47 g) in MeOH (300 mL) was stirred at 130° C. for 3 hours. The mixture was allowed to cool to RT, concentrated and purified to give 106-d (34.63 g). MS: m/z: 404[M+H]⁺.

To a solution of 106-d (32.46 g) and imidazole (21.97 g) in DMF (300 mL) was added tert-butyldimethylsilyl chloride (36.70 g) at 0° C. and the resulting mixture was stirred at 40° C. for 2 h. The mixture was diluted with EA and post processed to give 106-e (23.23 g). MS: m/z: 518[M+H]⁺.

A solution of 106-e (23.23 g) in methanol (300 ml) was added Pd/C (11.78 g), Pd(OH)₂/C (9.08 g) at H₂. The reaction mixture was stirred at 60° C. for 16 hours, filtered and the filtrate was concentrated to afford 106-f (9.39 g). MS: m/z: 248[M+H]⁺.

CP106-3 was synthesized in a manner similar to CP40-8 with CP106-f.

A solution of CP106-3 (2.47 g), p-TsOH·H₂O (2.17 g) in DMSO (30 mL) was stirred at 80° C. for 16 h. The mixture was allowed to cool to RT, diluted with water (50 mL), extracted with DCM and post processed to give CP106-4 (337 mg). MS: m/z: 370[M+H]⁺.

A solution of CP106-4 (0.337 g), triethylamine (792 mg), pyridine sulfur trioxide (610 mg) and DCM (3 mL) in DMSO (6 mL) was stirred at 25° C. for 16 h, quenched with Na₂S₂O₃, extracted with EA and post processed to give CP106-5 (190 mg). MS: m/z: 368[M+H]⁺.

To a solution of CP106-5 (0.19 g) in DCM (10 mL) was added DAST (3.36 g) at 0° C. and the resulting mixture was stirred at room for 16 h, quenched with saturated NaHCO₃(aq), extracted with DCM and post processed to give CP106-6 (132 mg). MS: m/z: 390[M+H]⁺.

CP106-8 was synthesized in a manner similar to CP46-10 with CP106-6.

CP106-8 was separated by Prep-HPLC-Gilson with the conditions: Column, CHIRAL ART Cellulose-SC column (2 cm×25 cm, 5 um); mobile phase, (Hex:DCM=3:1)(0.1% DEA)/EtOH (50:50); Flowing rate: 15 mL/min, 220 nm. This resulted in CP106-9A (the first eluting isomer, Retention time 4.67 min) and CP106-9B (the second eluting isomer, Retention time 6.98 min).

Compound 106A (CP106A, confirmed) was synthesized the similar procedures of the preparation of CP46 with CP106-9A. LCMS: m/z: 650[M+H]⁺.

Compound 106B (CP106B, confirmed) was synthesized the similar procedures of the preparation of CP46 with CP106-9B. LCMS: m/z: 650[M+H]⁺.

### Example 108

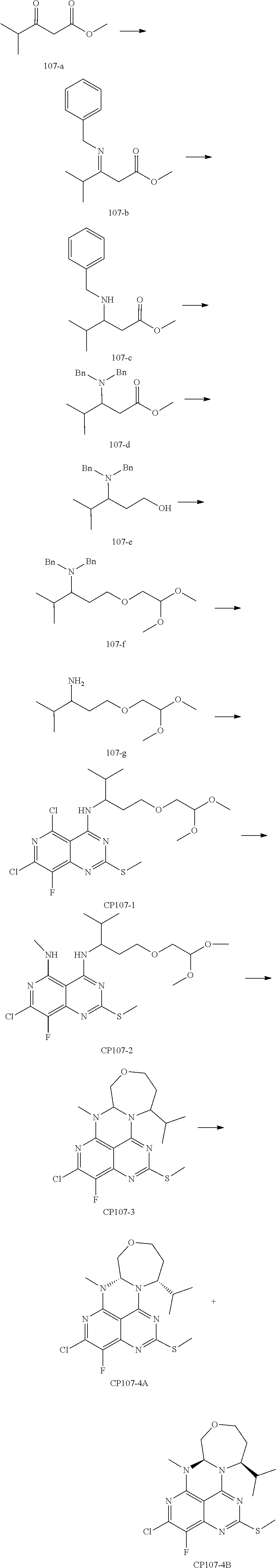

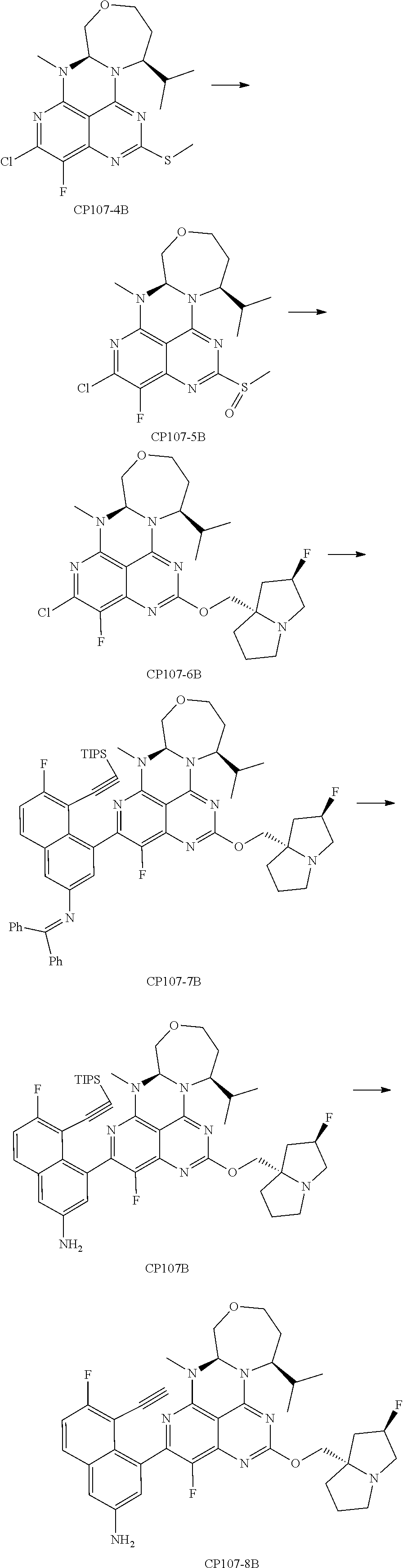

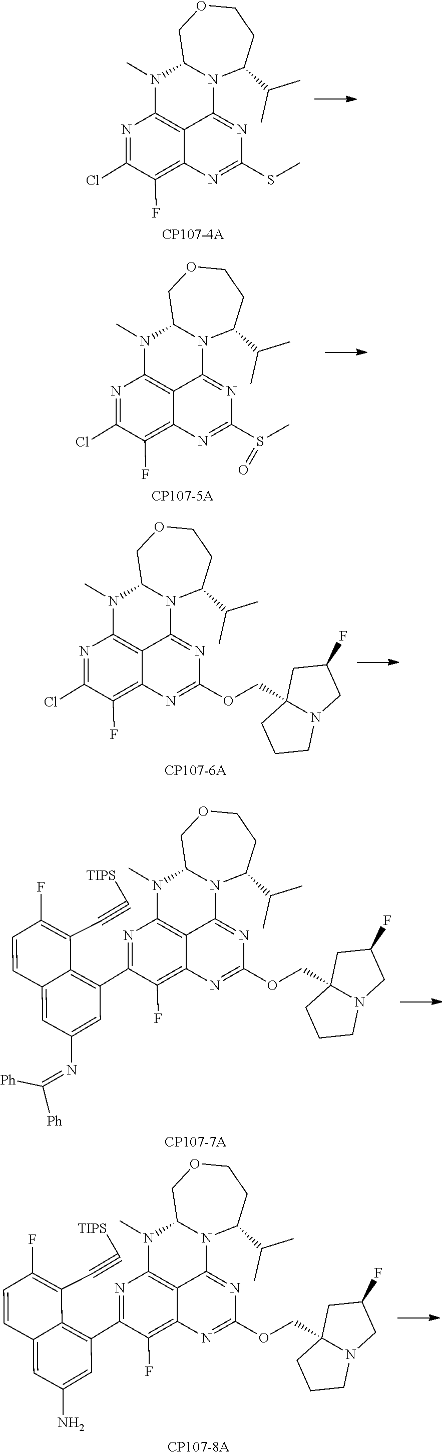

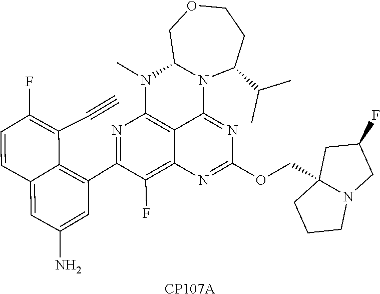 [SMILES: "CP107A" ; C#Cc1c(F)ccc2cc(N)cc(-c3nc4c5c(nc(OC[C@@]67CCCN6C[C@H](F)C7)nc5c3F)N3[C@@H](C(C)C)CCOC[C@H]3N4C)c12]

CP107-3 was synthesized in a manner similar to CP94-4 with CP17-a.

The crude CP107-3 (0.95 g) was separated by Prep-HPLC-Gilson with the conditions: Column, CHIRAL ART Cellulose-SC column (2 cm×25 cm, 5 un); mobile phase: (Hex:DCM=3:1)(0.1% DEA)/EtOH (50:50); Flowing rate: 15 mL/min, 220 nm. This resulted in CP107-4B (the first eluting isomer, Retention time: 4.17 min, 0.45 g) and CP107-4A (the second eluting isomer, Retention time: 7.193 min, 0.43 g).

Compound 107A (CP107A, 57.4 mg, confirmed) was synthesized in a manner similar to CP46 with CP107-4A. MS: m/z: 658[M+H]⁺.

Compound 107B (CP107B, 0.0708 g, confirmed) was synthesized in a manner similar to CP46 with CP107-4B. MS: m/z: 658[M+H]⁺.

### Example 109

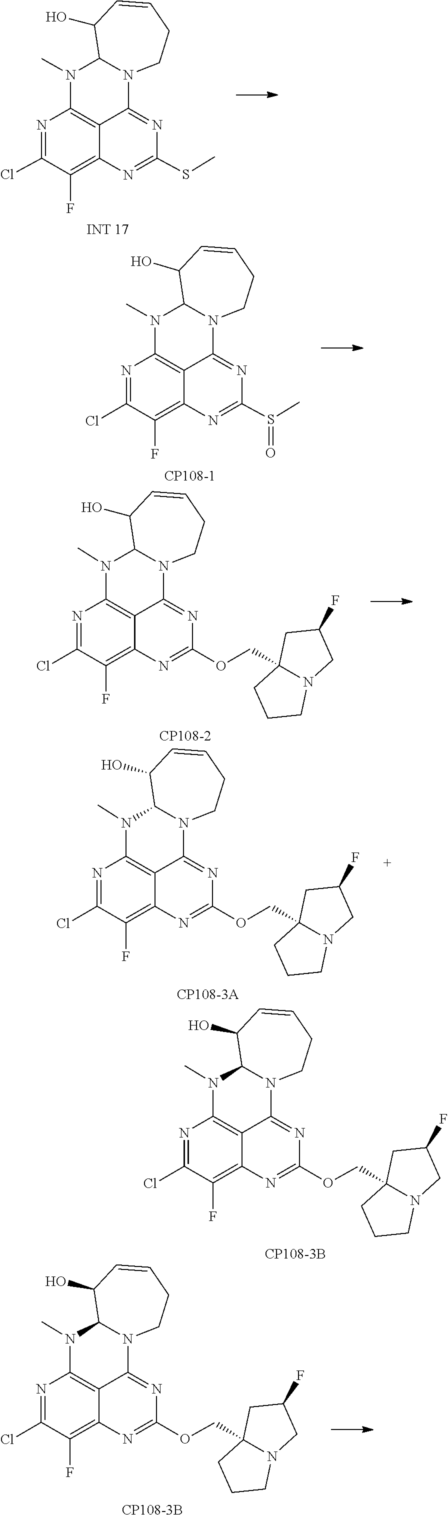

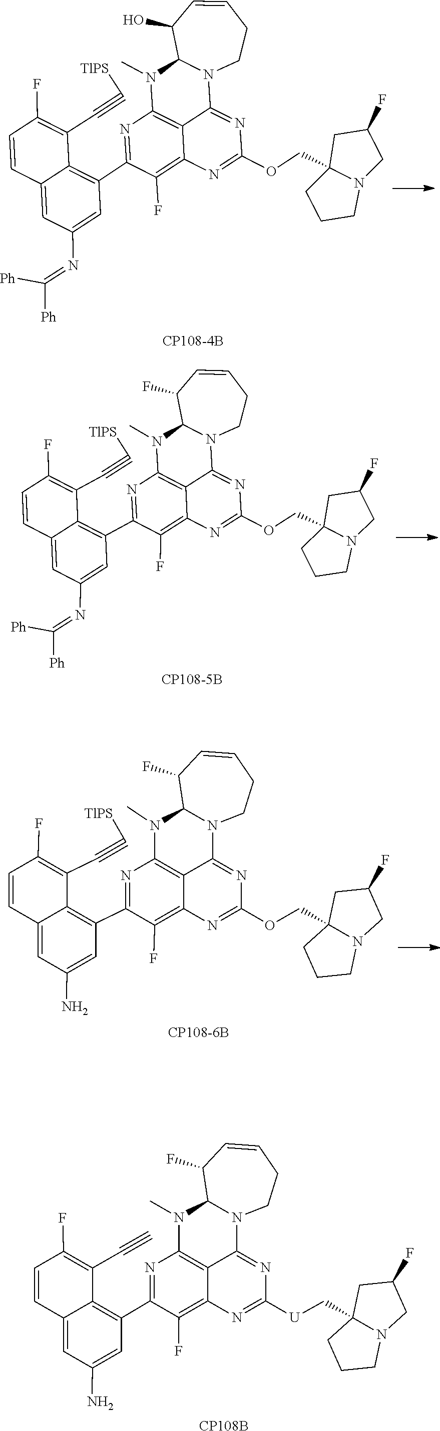

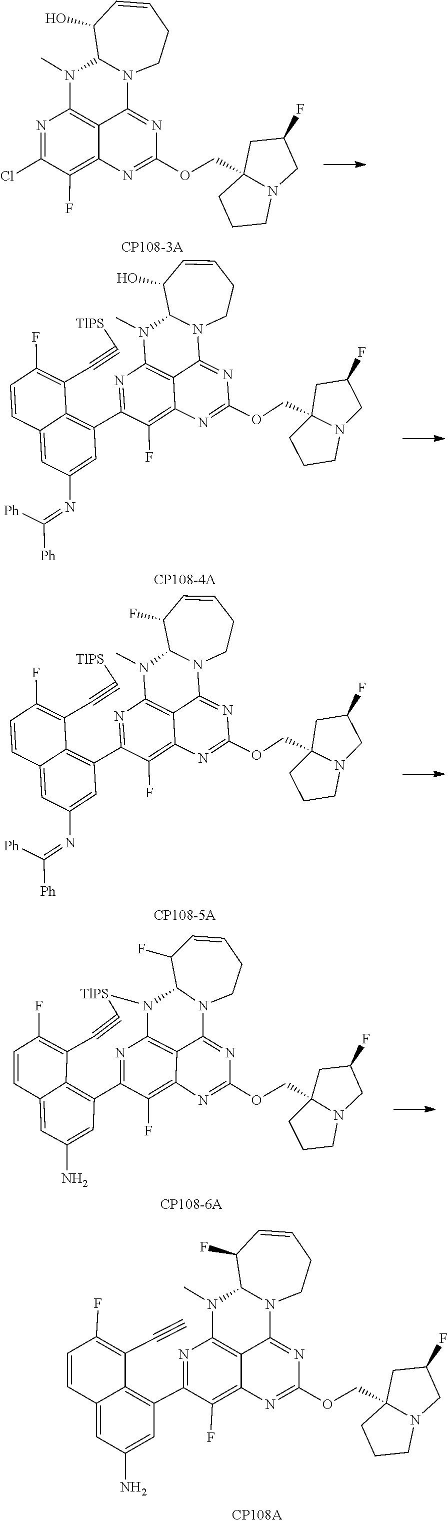

CP108-2 was synthesized in a manner similar to CP46-10 with INT17.

CP108-2 was separated by Prep-HPLC-Gilson with the conditions: Column, CHIRAL ART Cellulose-SC column (2 cm×25 cm, 5 um); mobile phase. (Hex:DCM=3:1)(0.1% DEA)/EtOH (50:50); Flowing rate: 15 mL/min, 220 nm. This resulted in CP108-3B (0.37 g, the first eluting isomer, Retention time 4.11 min) and CP108-3A (0.3 g, the second eluting isomer, Retention time 5.8 min). MS: m/z: 479[M+H]⁺.

CP108-4B was synthesized in a manner similar to CP46-11 with CP108-3B.

To a solution of CP108-4B (243.8000 mg) in THF (10 mL) was added DAST (0.41 g) at 0° C. under N₂ and the resulting mixture was stirred 2 h at RT. The mixture was diluted with sat. NaHCO₃, extracted with EA and post processed to afford CP108-5B (0.11 g). MS: m/z: 950[M+H]⁺.

Compound 108B (CP108B, confirmed) was synthesized the similar procedures of the preparation of CP46 with CP108-5B. LCMS: m/z: 630[M+H]⁺.

Compound 108A (CP108A, confirmed) was synthesized the similar procedures of the preparation of CP108B with CP108-3A. LCMS: m/z: 630[M+H]⁺.

### Example 110

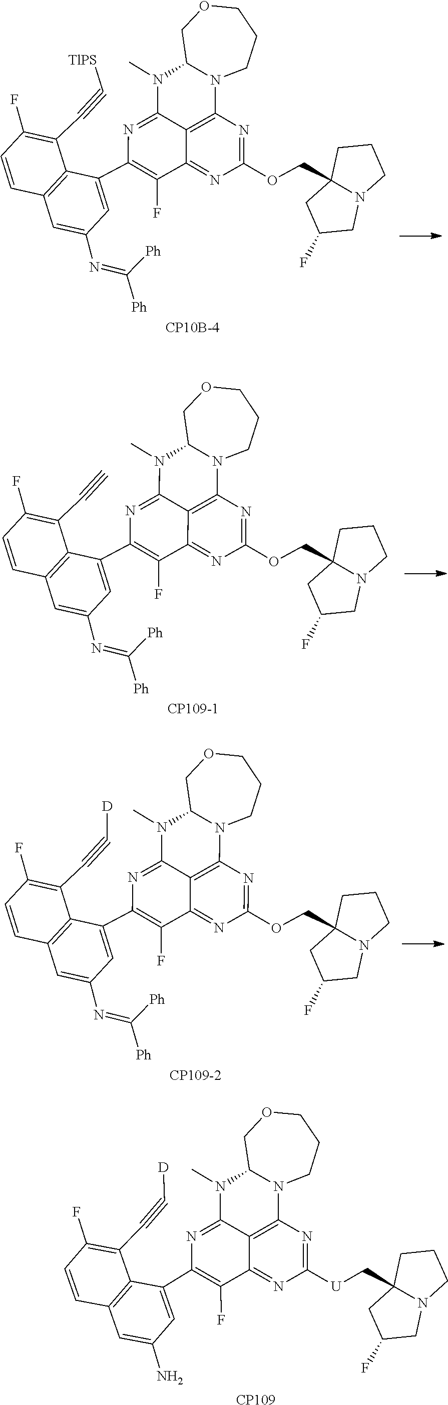

A solution of CP10B-4 (140 mg) and CsF (0.35 g) in DMF (3 mL) was stirred for 16 hours at RT under N₂. The solution was diluted with sat. aqueous NH₄Cl, extracted with EA and post processed to afford crude CP109-1 (186 mg). MS: m/z: 780[M+H]⁺.

A solution of crude CP109-1 (186 mg) in THF (5 mL) was cooled to −30° C. under N₂ and LDA (2M, 0.5 mL) was added dropwise at −30° C. The mixture was stirred at −30° C. for 30 min and CD₃OD (4 mL) was added at −30° C. and the resulting mixture was stirred at −30° C. for 1 h. The mixture was added D₂O and EA, post processed to afford crude CP109-2 (0.19 g). MS: m/z: 781[M+H]⁺.

To a solution of crude CP109-2 (179 mg) in DCM (10 mL), TEA (2 mL) was added. The mixture was stirred at RT for 1 h, diluted with 10% NaHCO₃ solution, extracted with DCM and post processed to afford Compound 109 (CP109, 74.3 mg, confirmed). ¹HNMR (400 MHz, DMSO-d₆) δ=7.80-7.67 (m, 1H), 7.34-7.28 (m, 1H), 7.03-6.98 (m, 2H), 5.59 (s, 2H), 5.40-5.17 (m, 2H), 4.55-4.38 (m, 1H), 4.20-3.91 (m, 4H), 3.62-3.47 (m, 1H), 3.46-3.22 (m, 2H), 3.16-3.00 (m, 3H), 3.02-2.92 (m, 3H), 2.86-2.80 (m, 1H), 2.32-2.10 (m, 2H), 2.08-1.95 (m, 3H), 1.90-1.69 (m, 3H). MS: m/z: 617[M+H]⁺.

### Example 111

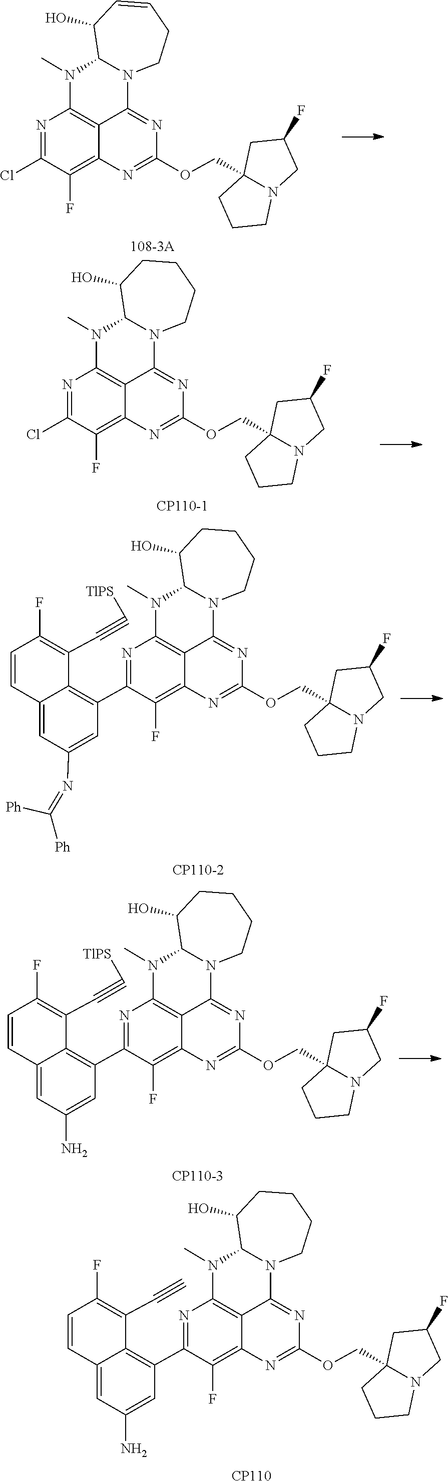

To a solution of 108-3A (0.09 g) in CH₃OH (10 mL) and DCM (30 mL) was added Pd/C (0.0373 g). The mixture was stirred under H₂ at RT for 3 h. The solution was filtered and the filtrate was concentrated to afford CP110-1 (0.0823 g). MS: m/z: 481[M+H]⁺.

Compound 110 (CP110, confirmed) was synthesized the similar procedures of the preparation of CP46 with CP110-1. LCMS: m/z: 630[M+H]⁺.

### Example 112

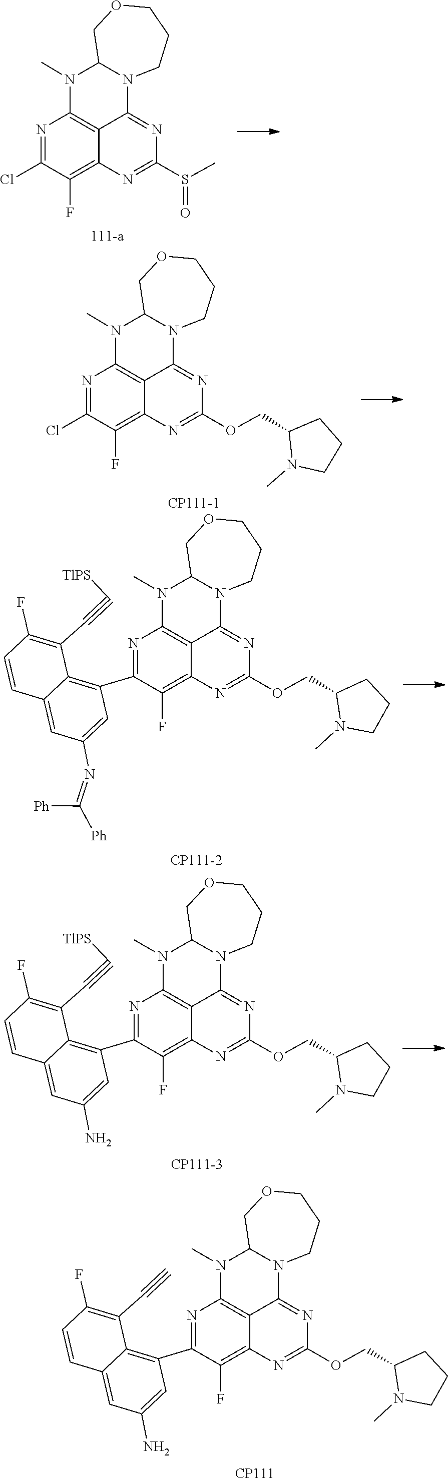

Compound 111 (CP111, confirmed) was synthesized in a manner similar to CP46. MS: m/z: 572[M+H]⁺. ¹HNMR (400 MHz, DMSO-d₆) δ=7.80-7.68 (m, 1H), 7.34-7.28 (m, 1H), 7.05-6.95 (m, 2H), 5.58 (s, 2H), 5.29-5.21 (m, 1H), 4.53-4.31 (m, 2H), 4.30-4.14 (m, 1.5H), 4.05-3.88 (m, 2.5H), 3.62-3.39 (m, 3H), 3.08-2.93 (m, 4H), 2.80-2.68 (m, 1H), 2.46-2.37 (m, 3H), 2.35-2.18 (m, 2H), 2.05-1.99 (m, 2H), 1.78-1.60 (m, 3H).

### Example 113

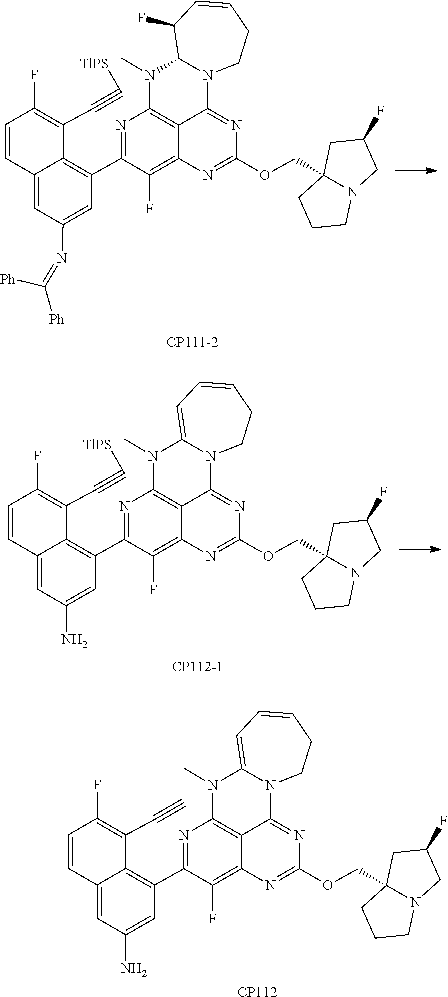

A solution of CP108-5A (0.23 g) and HCl in dioxane (4M, 2 mL) in DCM (8 mL) was stirred at RT for 1 h. The solution was diluted with 10% h NaHCO₃ solution, extracted with DCM and post processed to give crud CP112-1 (0.10 g). MS: m/z: 766[M+H]⁺.

A solution of CP112-1 (0.1 g) and CsF (0.21 g) in DMF (5 mL) was stirred for 2 hours at RT under N₂. The solution was diluted with sat. NaHCO₃, extracted with EA and post processed to afford Compound 112 (CP112, 0.0034 g, confirmed). MS: m/z: 610[M+H]⁺.

### Example 114

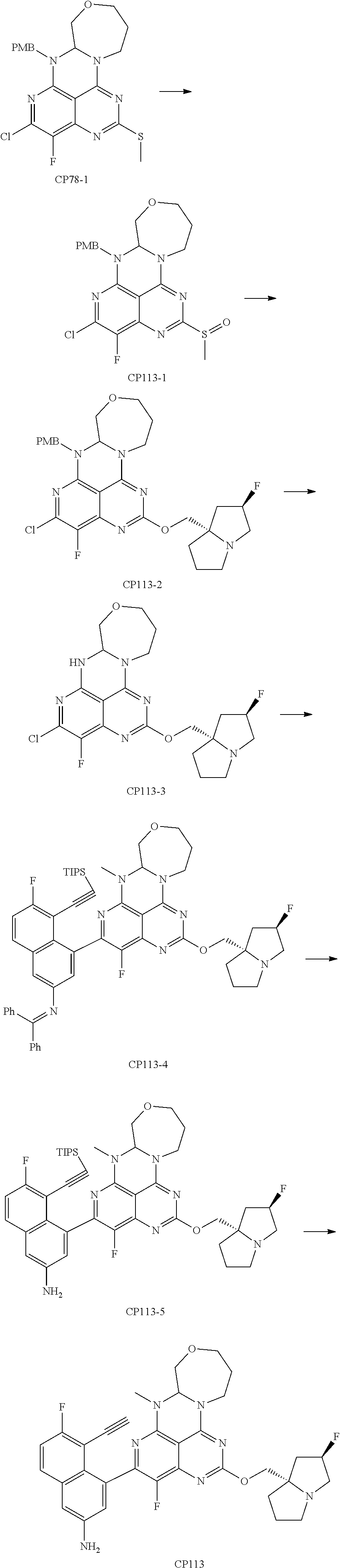

To a solution of CP78-1 (79 mg) in DCM (10 mL) was added m-CPBA (66 mg) at RT. The resulting mixture was stirred for 1 hour, diluted with DCM and washed with aq. NaHCO₃ and post processed to afford CP113-1 (87 mg). MS (ESI, m/z): 478[M+H]⁺.

To a solution of CP113-1 (87 mg) in 1,4-dioxane (5 mL) were added N,N-diisopropylethylamine (82 mg) and INT14 (69 mg). The reaction mixture was stirred at 90° C. for 16 hours, diluted with EA and post processed to afford CP113-2 (54 mg). MS (ESI, m/z): 573[M+H]⁺.

A solution of CP113-2 (54 mg) in TFA (4 mL) was stirred at RT for 16 hours and concentrated. The residue was dissolved in EA, washed with aq. NaHCO₃, dried, concentrated and purified to afford CP113-3 (33 mg). MS (ESI, m/z): 453[M+H]⁺.

Compound 113 (CP113, confirmed) (13.1 mg, TFA salt) was synthesized the procedure of CP46 with CP113-3. MS (ESI, m/z): 602[M+H]⁺.

### Example 115

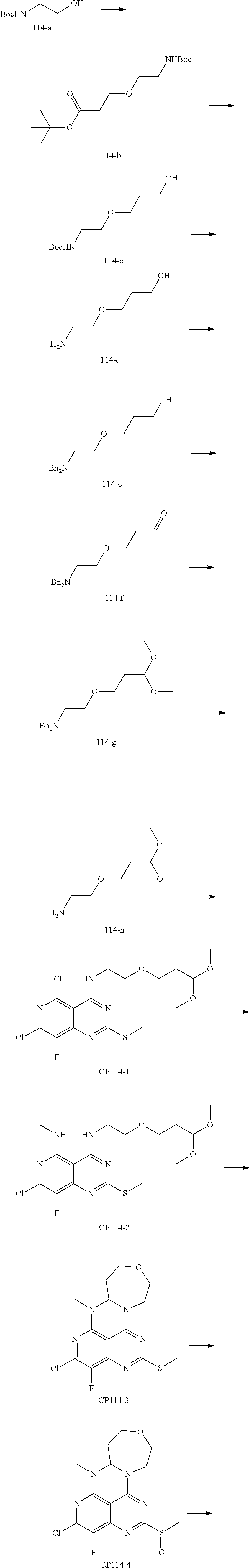

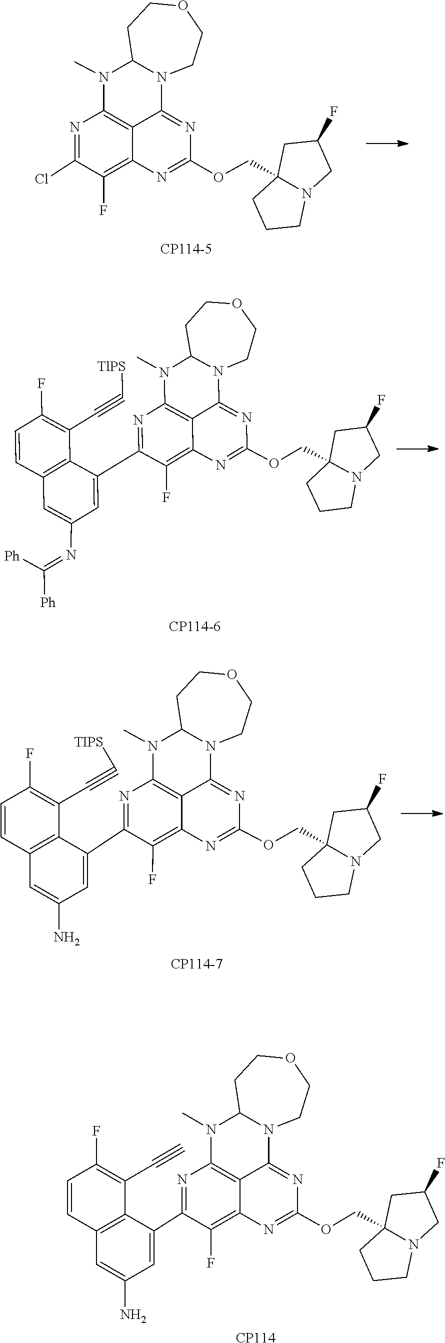

To a solution of 114-a (5.02 g) and tert-butyl acrylate (8.04 g) in 1,4-dioxane (25 mL) was added 60% KOH solution (aq, 1 mL) and the resulting mixture was stirred at RT for 20 hours, diluted with water, extracted with EA and post processed to afford 114-b (8.88 g). MS (ESI, m/z: 290[M+H]⁺.

To a solution of 114-b (8.88 g) in THF (150 mL) was added LAH (1.70 g) in portions at 0° C. and the resulting mixture was stirred at RT for 2 hours, quenched by water, aq. 15% NaOH solution and water in order. The mixture was filtered and the filtrate was concentrated to afford 114-c (6.29 g). MS (ESI, m/z): 220[M+H]⁺.

To a solution of 114-c (5.946 g) in acetonitrile (60 mL) was added HCl (4M in 1,4-dioxane, 20 mL). The reaction mixture was stirred at RT for 5 hours and concentrated to afford 114-d which was used in next step directly. MS (ESI, m/z): 120[M+H]⁺.

Compound 114 (CP114, confirmed) was synthesized in a manner similar to CP96 with CP14-d. MS (ESI, m/z): 616[M+H]⁺.

### Example 116

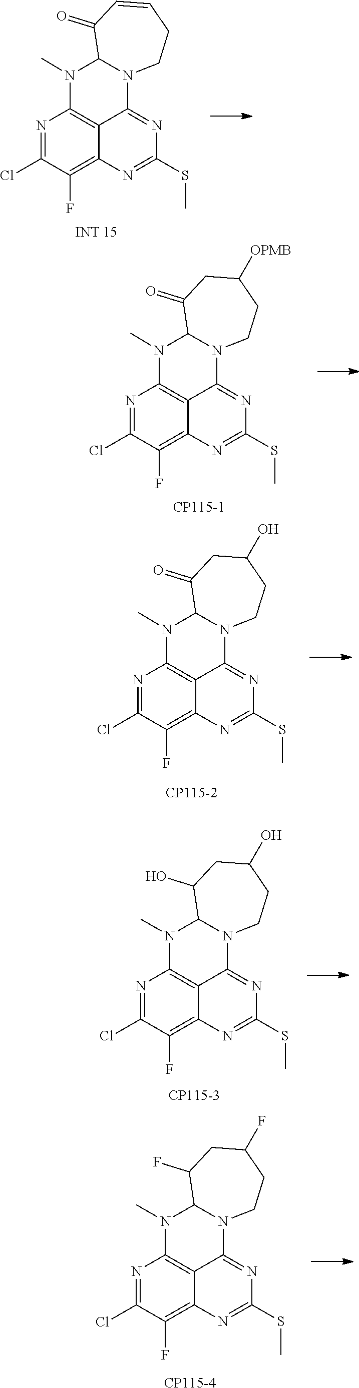

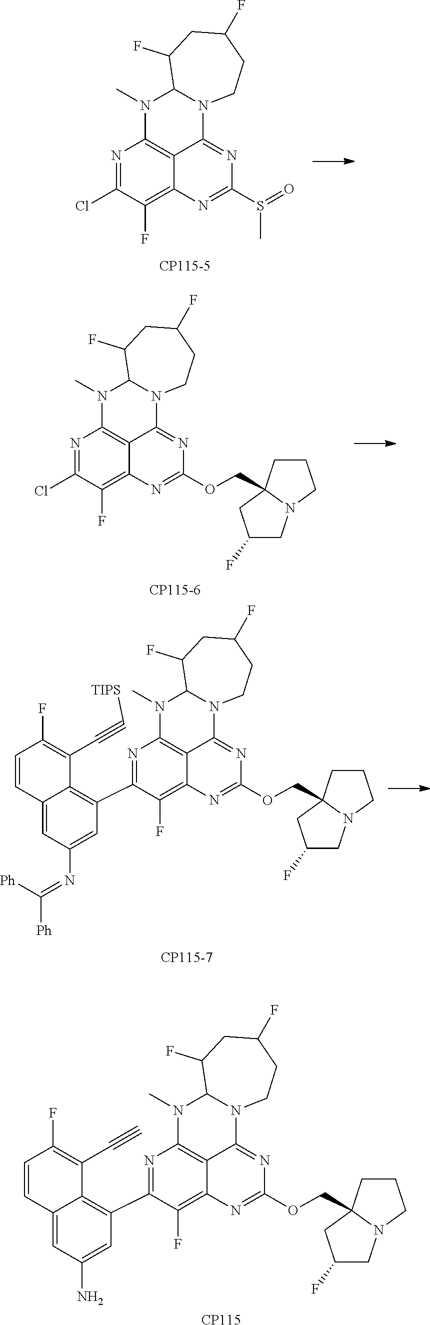

To a solution of INT15 (619 mg) in (4-methoxyphenyl)methanol (5 mL) was added t-BuOK (60 mg). The resulting mixture was stirred at RT for 3 hours and then purified to give CP115-1 (0.36 g). MS: m/z: 504[M+H]⁺.

A solution of CP115-1 (0.36 g) in TFA (3 mL) was stirred at RT for 4 hours and then concentrated to afford CP115-2 which was used in the next step directly. MS: m/z: 384[M+H]⁺.

To a solution of CP115-2 (442 mg) in THF (10 mL) and DCM (5 mL) was added NaBH₄ (0.10 g) at 0° C. The mixture was stirred at RT overnight, diluted with water and EA and post processed to give CP115-3 (35 mg). MS: m/z: 386[M+H]⁺.

To a solution of CP115-3 (35 mg) in DCM (5 mL) was added DAST (85 mg) at 0° C. The mixture was stirred at RT for 2 hours. The reaction mixture was diluted with sat.aq. NaHCO₃ and EA, and post processed to give CP115-4 (9 mg). MS: m/z: 390[M+H]⁺.

Compound 115 (CP115, free base, 2.3 mg, confirmed) was synthesized the procedure of CP46 with CP115-4. LCMS: m/z: 650[M+H]⁺.

### Example 117

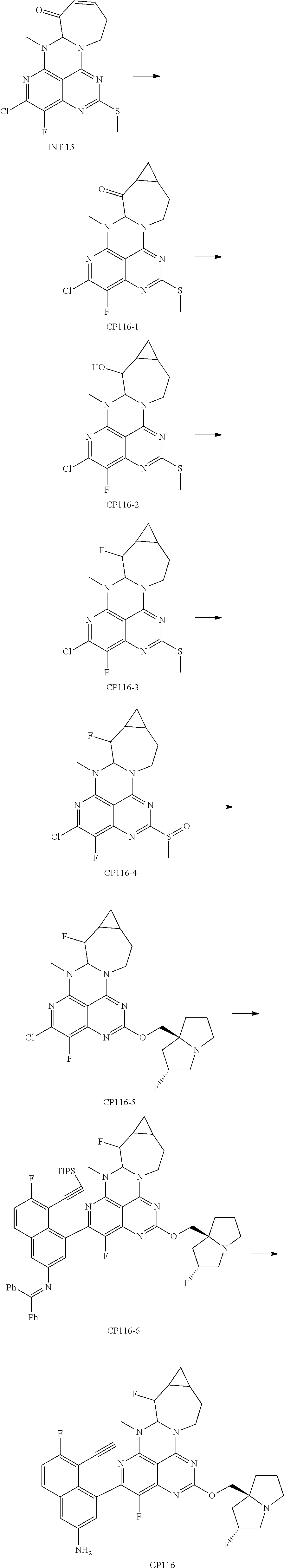

To a solution of NaH (65 mg, 60% content) in DMSO (10 mL) was added trimethylsulfoxonium iodide (330 mg) in portions under N₂. The mixture was stirred at RT for 3 hours. INT15 (0.5 g) was added in portions and the mixture was stirred at RT for 3.5 hours, diluted with water and EA and post processed to give CP116-1 (279 mg). MS: m/z: 380[M+H]⁺.

To a solution of CP116-1 (263 mg) in THF (6 mL) and DCM (3 mL) was added NaBH₄ (70 mg). The mixture was stirred at RT overnight, diluted with water and EA and post processed to give CP116-2 (284 mg). MS: m/z: 382[M+H]⁺.

To a solution of CP116-2 (237 mg) in DCM (20 mL) was added DAST (511 mg) at 0° C. The mixture was stirred at RT for 1.5 hours, diluted with DCM and water and post processed to give CP116-3 (80 mg). MS: m/z: 384[M+H]⁺.

CP116 (28.6 mg, free base, confirmed) was synthesized in a manner similar to CP46 with CP116-3. LCMS: m/z: 644[M+H]⁺.

### Example 118

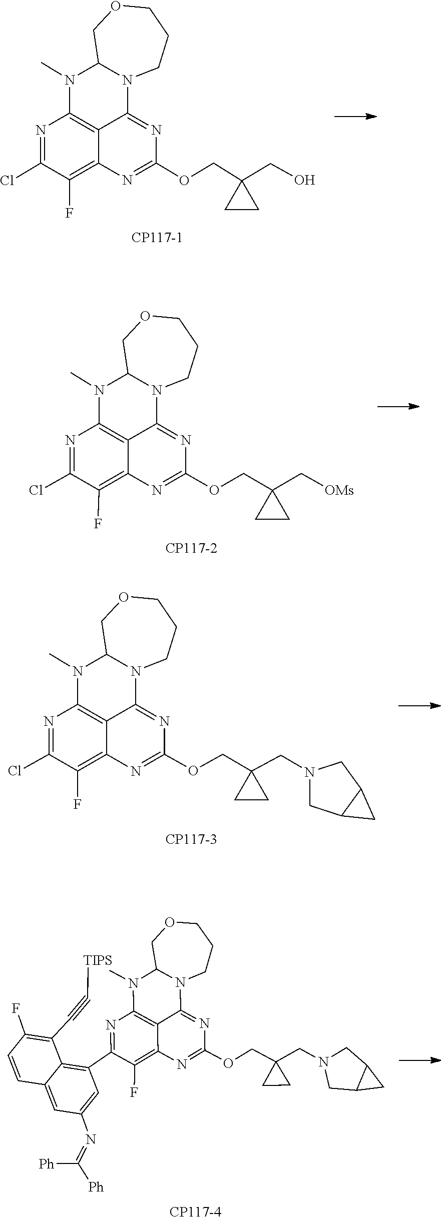

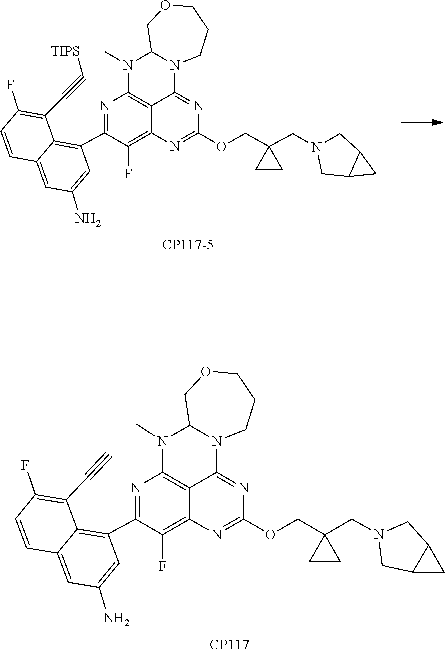

Compound 117 (CP117, confirmed) was synthesized in a manner similar to CP9. LCMS: m/z: 624[M+H]⁺.

### Example 119

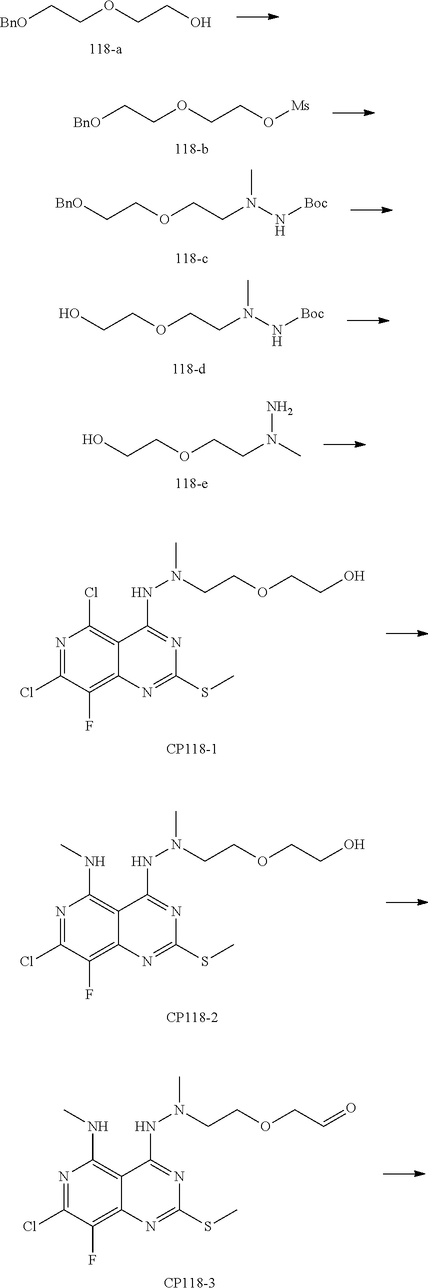

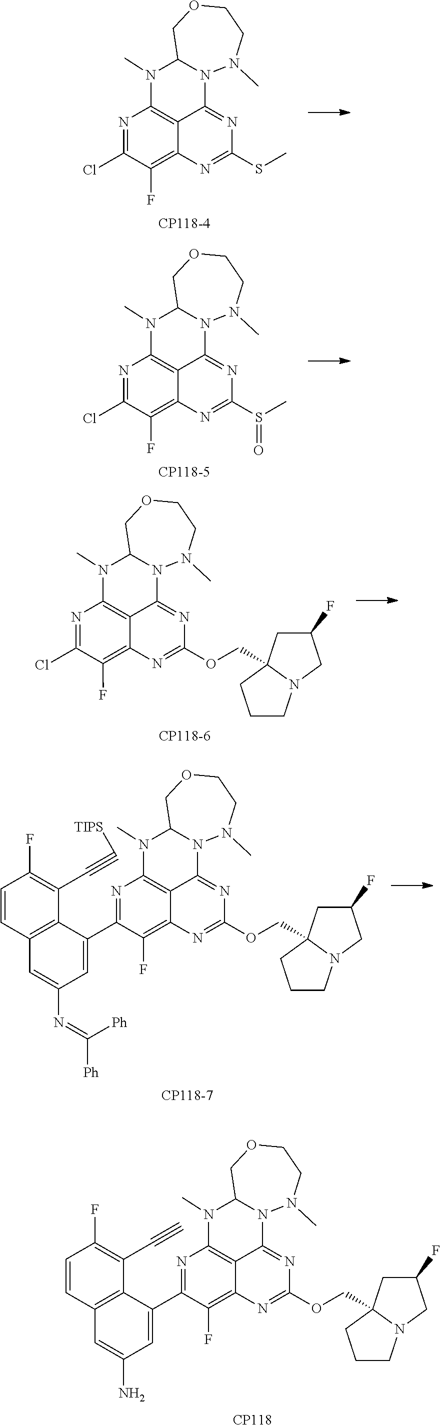

To a solution of 118-a (6.21 g), TEA (8.91 g) in DCM (40 mL) was added MsCl (3.96 g) dropwise at −10° C. The reaction mixture was stirred for 15 mins at −10° C., quenched with water, extracted with DCM and post processed to give 118-b (8.35 g).

To a solution of 118-b (8.35 g) in DMF (50 mL) were added DIEA (11.38 g) and tert-butyl 2-methylhydrazine-1-carboxylate (4.69 g). The reaction mixture was stirred at 80° C. for 22 hours, diluted with EA and water and post processed to give 118-c (8.78 g). MS: m/z: 325[M+H]⁺.

To a solution of 118-c (8.78 g) in MeOH (100 mL) were added Pd/C (2.27 g, 10% wt Pd content) and Pd(OH)₂/C (2.00 g, 7.6% wt Pd content). The reaction mixture was stirred at RT for 19 hours under H₂, filtered and the filtrate was concentrated to give 118-d (5.84 g). MS: m/z: 235[M+H]⁺.

To a solution of 118-d (1.51 g) in DCM (15 mL) was added TFA (6 mL). The reaction mixture was stirred at RT for 3 hours and concentrated. The residue was dissolved in MeOH (30 mL) and the pH value of the solution was adjusted to 13 with NaOH solid. After stirring for 2.5 hours, the pH value of the mixture was adjusted to 2 with HCl (4M in dioxane). The reaction mixture was concentrated to give 118-e (crude). MS: m/z: 135[M+H]⁺.

Compound 118 (CP118, 2.6 mg, TFA salt, confirmed) was synthesized the procedure of CP47 with 118-e. MS: m/z: 631[M+H]⁺.

### Example 120

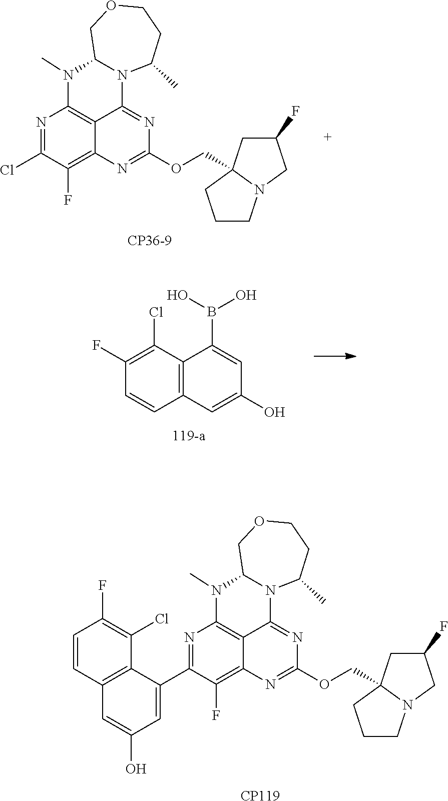

To a solution of CP36-9 (0.35 g), 119-a (synthesized according to the procedure described in WO2022042630, 293 mg) in toluene (20 mL) and water (5 mL) were added cataCxium A Pd G3 (66 mg) and Cs₂CO₃ (692 mg). The reaction mixture was stirred at 100° C. for 20 hours under N₂, diluted with water, extracted with EA and post processed to give Compound 119 (CP119, 310 mg, confirmed). MS: m/z: 641[M+H]⁺.

### Example 121

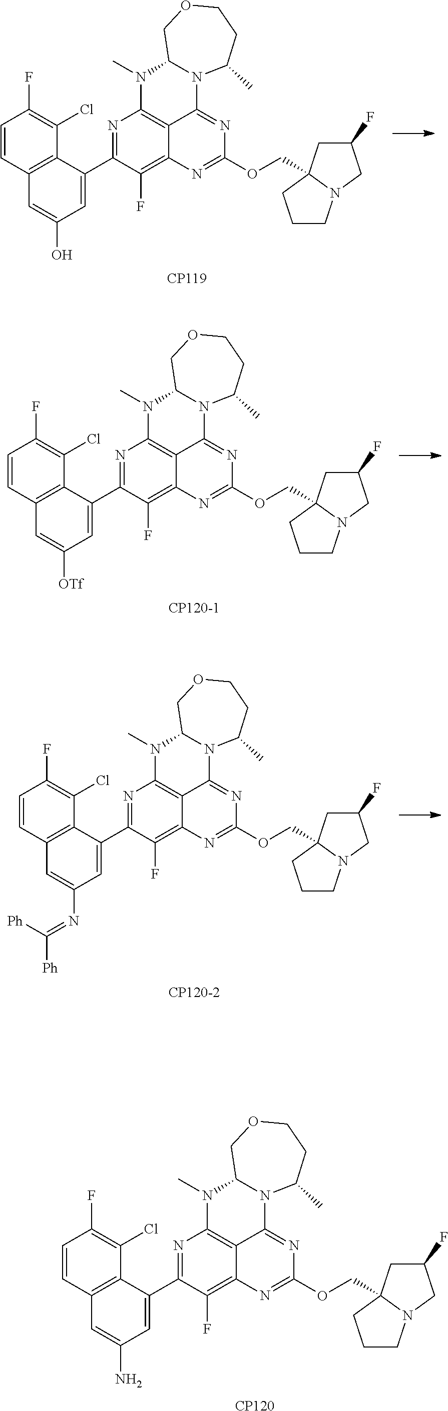

Compound 120 (CP120, confirmed) was synthesized in a manner similar to CP1 with CP119. LCMS: m/z: 640[M+H].

### Example 122

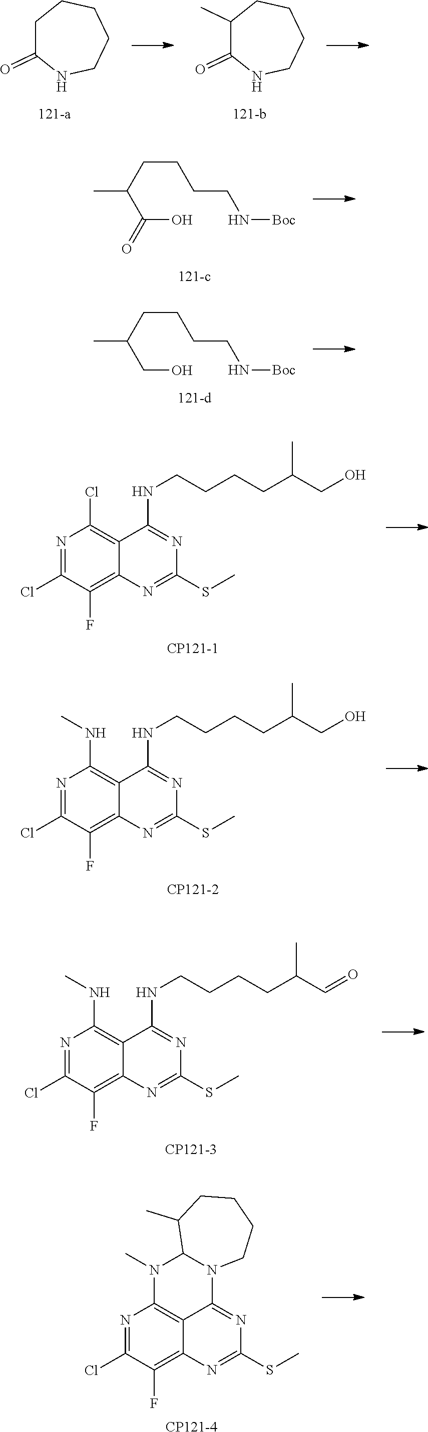

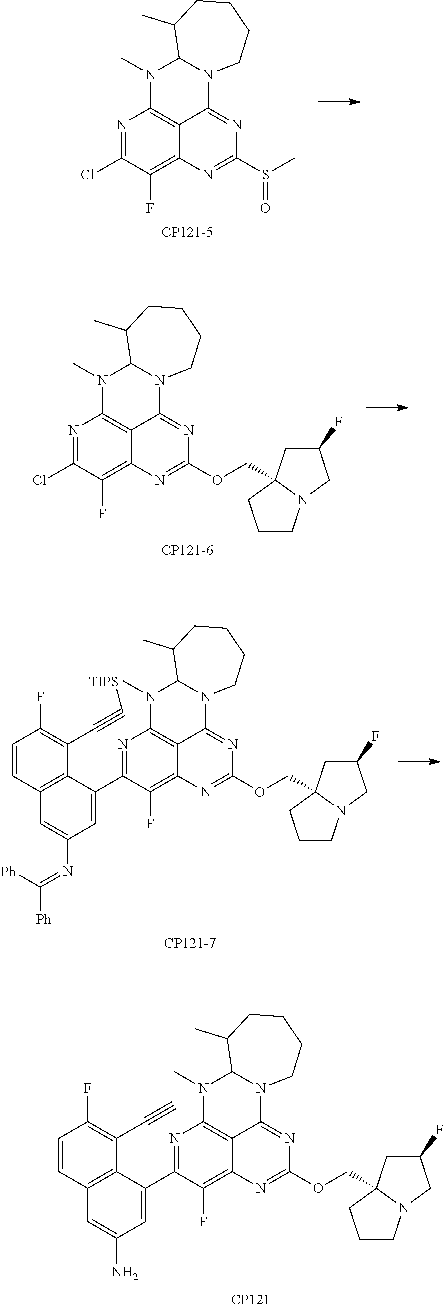

To a solution of 121-a (2.06 g) in THF (8(mL) was added n-BuLi (17.6 mL, 2 mol/L in hexane) dropwise at −5° C. After stirring for 1.5 hours at −10° C., a solution of iodomethane (2.80 g) in THF (4 mL) was added dropwise at −5° C. The reaction mixture was stirred for 3 hours at ° C., quenched with aq. NaHCO₃ (50 mL, 10% wt), extracted with EA and post processed to give 121-b (2.24 g). MS: m/z: 128[M+H]⁺.

To a mixture of 121-b (2.24 g) in water (4 mL) was added KOH (5.02 g). After stirring at 100° C. for 6.5 hours, the reaction mixture was cooled to RT, (Boc)₂O (4.61 g) was added. The reaction mixture was stirred for 4 hours at RT, diluted with EA and water and post processed to give 121-c (8.78 g). MS: m/z: 246[M+H]⁺.

To a solution of 121-c (3.42 g) in THF (50 mL) was added LAH (1.21 g) in portions at −10° C. The reaction mixture was stirred at RT for 2 hours, quenched with water (1.5 mL), aq. NaOH (1.5 mL, 15a/wt) and water (5 mL) in order, filtered and the filtrate was concentrated to give 121-d (1442 mg). MS: m/z: 232[M+H]⁺.

To a solution of 121-d (1442 mg) in acetonitrile (20 mL) was added HCl 1 (10 mL, 4M in dioxane), stirred at RT for 1 hour, concentrated to afford residue A.

To a mixture of INT6 (1.53 g), DIEA (2.18 g) in toluene (15 mL) was added POCl₃ (0.75 mL). The reaction mixture was stirred at 80° C. for 1 hour and concentrated to afford residue B. The pH of a mixture of the residue A in DCM (10 mL) was adjusted to alkaline with DIEA. To a mixture of the residue B in DCM (20 mL) was added DIEA (3 mL) and the mixture above. The reaction mixture was stirred at RT for 2 hour, diluted with water and extracted with DCM and post processed to give CP121-1 (3.03 g). MS: m/z: 393[M+H]⁺.

Compound 121 (CP121, 44.3 mg, TFA salt, confirmed) was synthesized in a manner similar to CP47 with CP121-1. MS: m/z: 628[M+H]⁺.

### Example 123

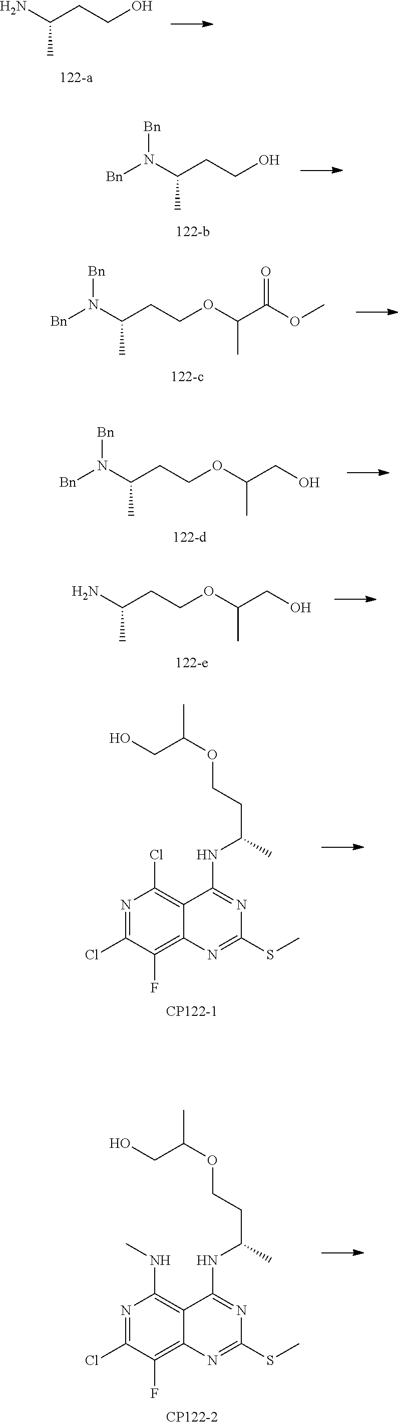

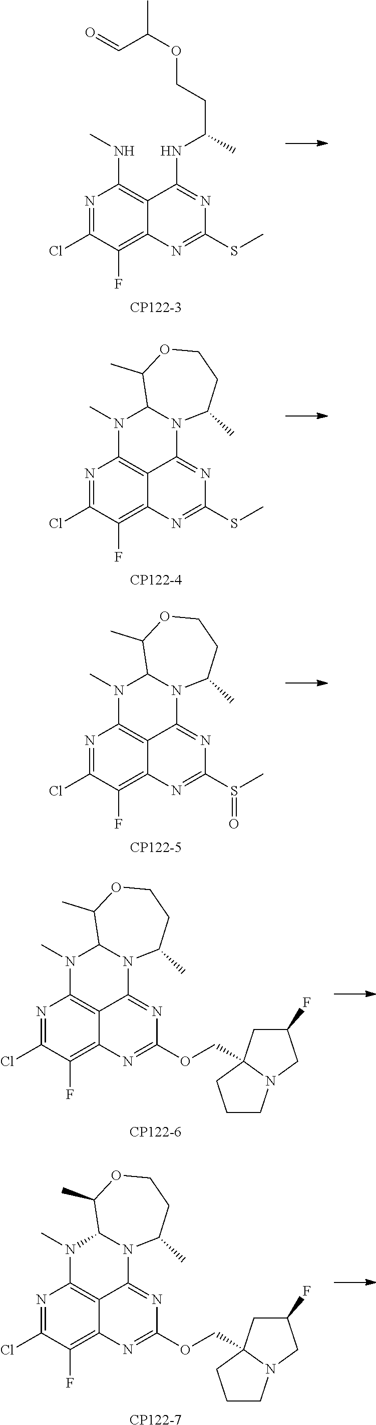

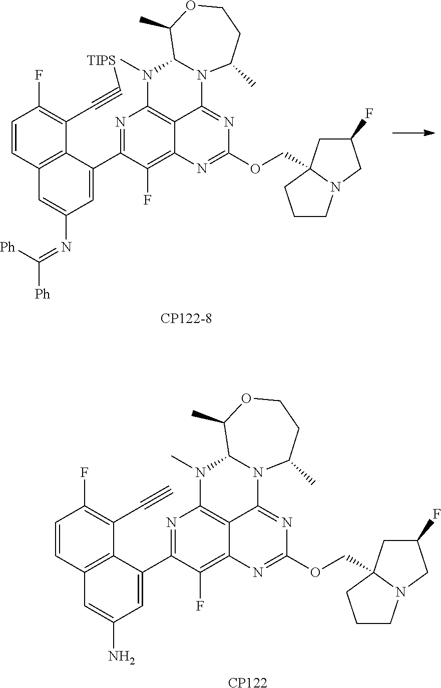

To a solution of 122-a (5.06 g) in acetonitrile (100 mL) were added K₂CO₃ (22.73 g), tetrabutylammonium iodide (1.95 g) and benzyl bromide (23.89 g). The reaction mixture was stirred for 20 hours at RT, filtered and the filtrate was concentrated and purified to give 122-b (14.35 g). MS: m/z: 270[M+H]⁺.

To a solution of 122-b (2.08 g) in THF (20 mL) was added NaH (0.81 g, 60% content in oil) at −10° C. After stirring for 15 mins, methyl 2-bromopropionate (1.56 g) was added to the mixture. The reaction mixture was stirred for 2.5 hours at RT, quenched with water, extracted with EA and post processed to give 122-c (2.26 g). MS: m/z: 356[M+H]⁺.

To a solution of 122-c (2.26 g) in THF (30 mL) was added LAH (0.40 g) in portions at −10° C. The reaction mixture was stirred at RT for 2 hours, quenched with water (0.5 mL), aq. NaOH (0.5 mL, 15% wt) and water (1.5 mL) in order. The mixture was filtered and the filtrate was concentrated and purified to give 122-d (1.94 g). MS: m/z: 328[M+H]⁺.

To a solution of 122-d (1.94 g) in MeOH (30 mL) were added Pd/C (0.56 g, 10% wt Pd content) and Pd(OH)₂/C (0.53 g, 7.6% wt Pd content). The reaction mixture was stirred at RT for 21 hours under H₂, filtered and the filtrate was concentrated to give 122-e (0.65 g). MS: m/z: 148[M+H]⁺.

Compound 122 (CP122, 7 mg. TFA salt, confirmed) was synthesized in a manner similar to CP47 with CP122-e. MS: m/z: 644[M+H]⁺.

### Example 124

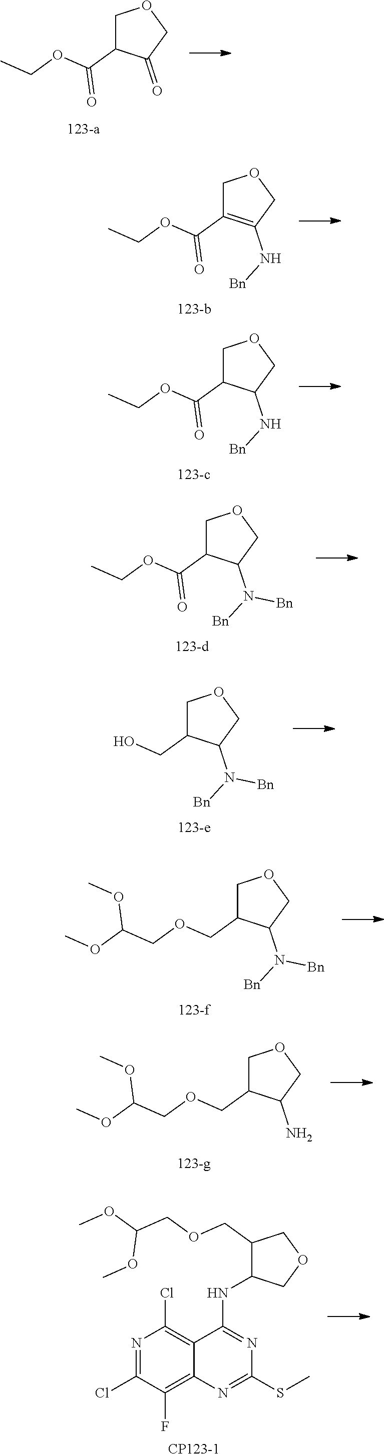

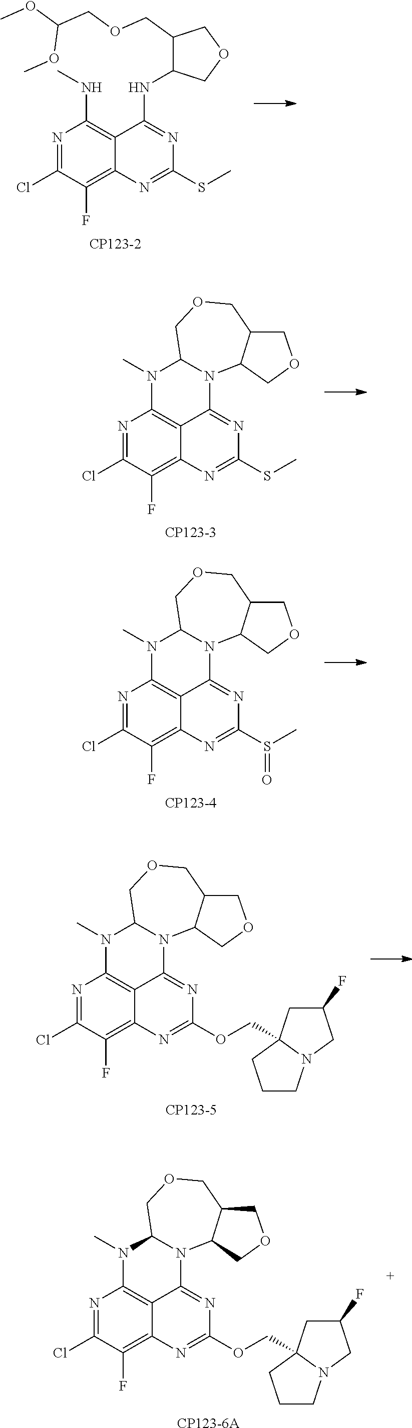

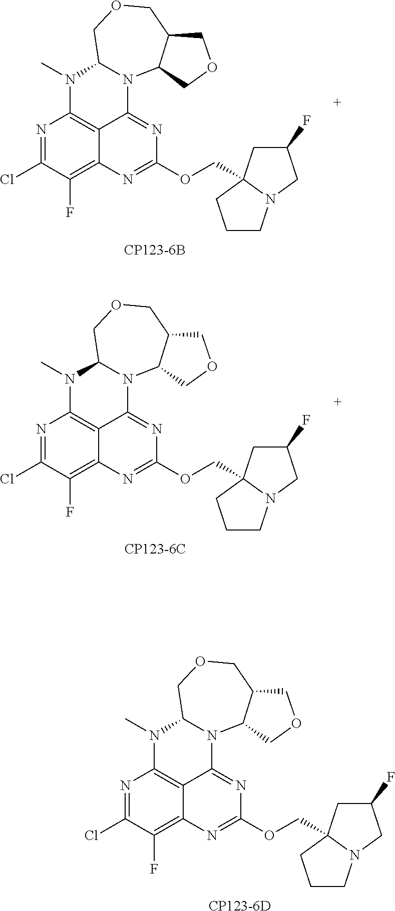 [SMILES: CN1c2nc(Cl)c(F)c3nc(OC[C@@]45CCCN4C[C@H](F)C5)nc(c23)N2[C@@H]3COC[C@@H]3COC[C@@H]12 ; "CP123-6B" ; CN1c2nc(Cl)c(F)c3nc(OC[C@@]45CCCN4C[C@H](F)C5)nc(c23)N2[C@@H]1COC[C@@H]1COC[C@@H]12 ; "CP123-6C" ; CN1c2nc(Cl)c(F)c3nc(OC[C@@]45CCCN4C[C@H](F)C5)nc(c23)N2[C@H]3COC[C@H]3COC[C@@H]12 ; "CP123-6D"]

CP123-5 was synthesized in a manner similar to CP94-7 with CP123-a.

The first peak of CP123-5 (64 mg) was separated by Prep-HPLC-Gilson with the conditions: Column, CHIRAL ART Cellulose-SA column (2 cm×25 cm, 5 μm); mobile phase, (Hex:DCM=3:1) (0.1% DEA)/EtOH (50:50); Flow rate: 20 mL/min. This resulted in CP123-5A (34 mg, the first eluting isomer, Retention time: 5.71 min) and CP123-5B (28 mg, the second eluting isomer, Retention time: 7.26 min).

The second peak CP123-5 (70 mg) was separated by Prep-HPLC-Gilson with the conditions: Column, CHIRAL ART Cellulose-SC column (2 cm×25 cm, 5 um); mobile phase, (Hex:DCM=3:1)(0.1% DEA)/EtOH (50:50); Flow rate: 15 mL/min, 220 nm. This resulted in CP123-SC (23 mg the first eluting isomer, Retention time: 6.897 min) and CP123-5D (19 mg, the second eluting isomer, Retention time: 14.551 min).

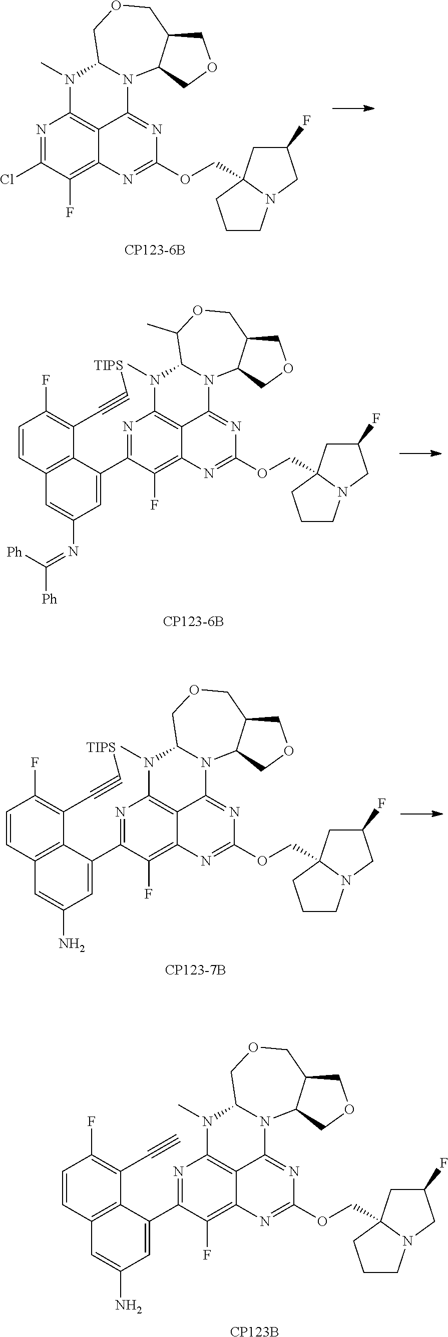

Compound 123B (CP123B, assumed) was synthesized in a manner similar to CP46 with CP123-6B. MS: m/z: 658[M+H]⁺.

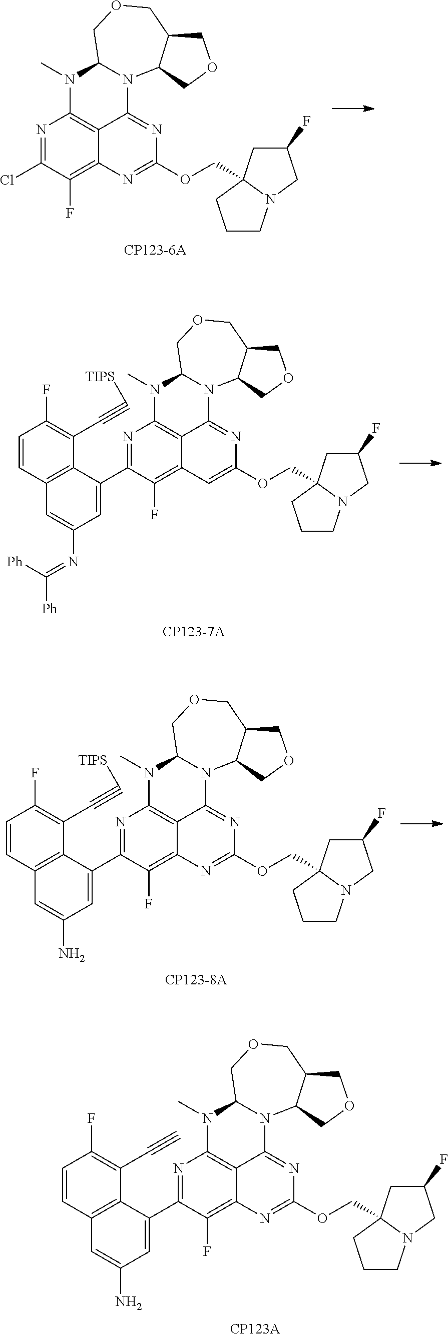

Compound 123A (CP123A, 7.8 mg, assumed) was synthesized in a manner similar to CP123B with CP123-6A. MS: m/z: 658[M+H]⁺.

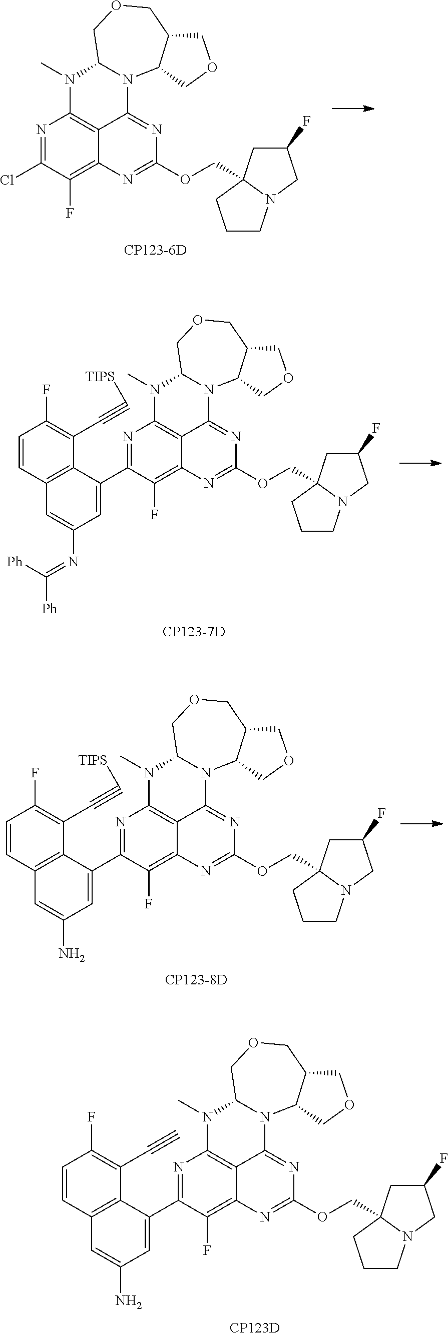

Compound 123D (CP123D, 5.4 mg, assumed) was synthesized in a manner similar to CP123B with CP123-6D. MS: m/z: 658[M+H]⁺.

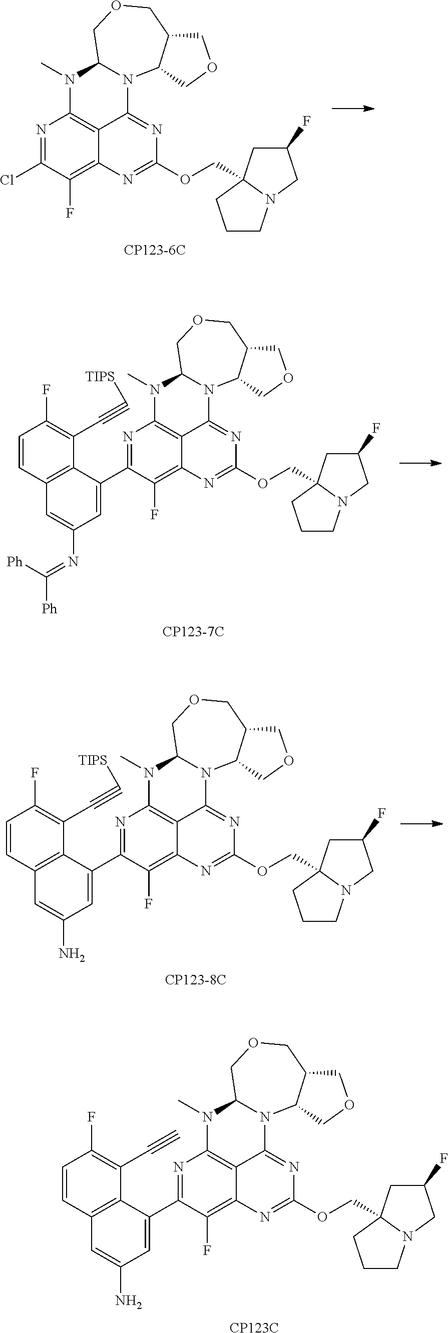

Compound 123 (CP123, 6.6 mg, assumed) was synthesized in a manner similar to CP123-6B with CP123-6C. MS: m/z: 658[M+H]⁺.

### Example 125

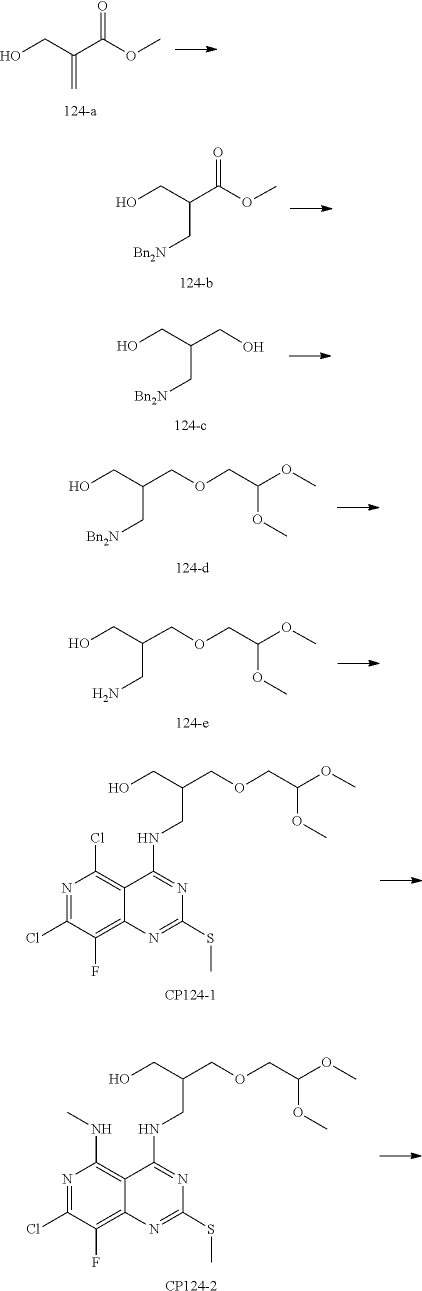

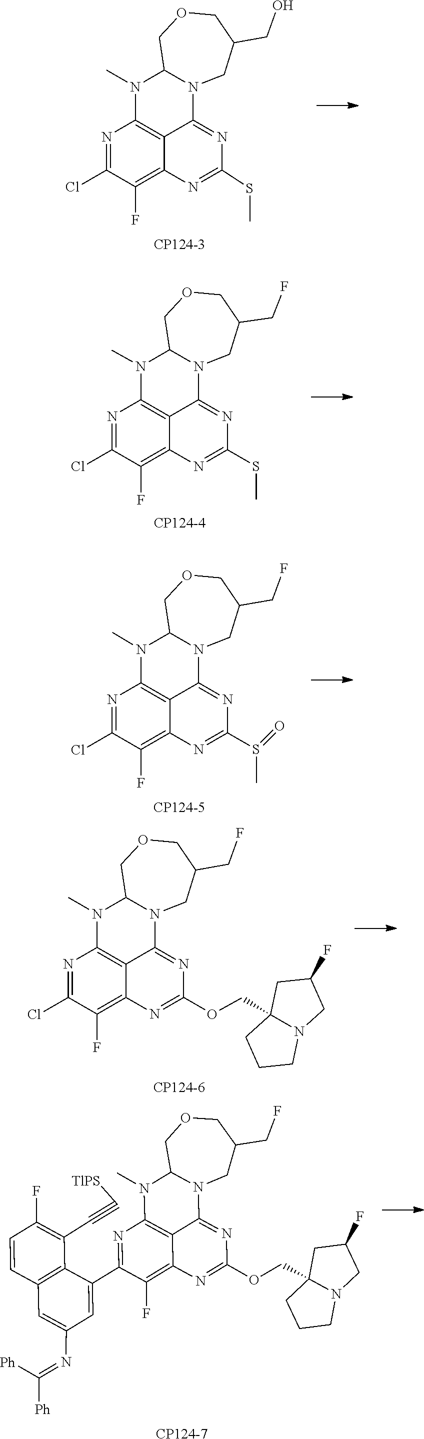

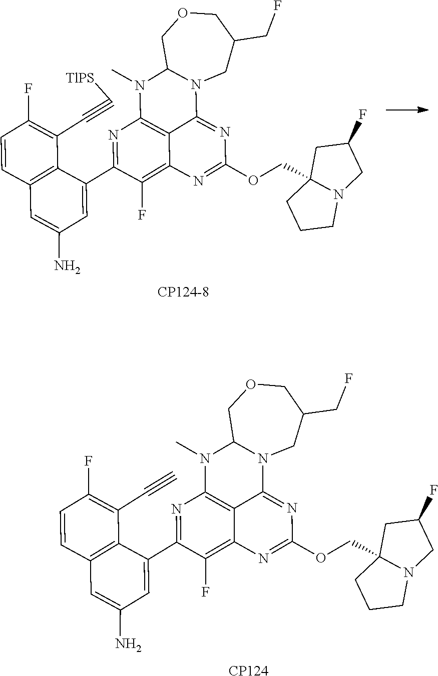

A solution of 124-a (3.00 g), dibenzylamine (25.03 g) and DBU (8.05 g) in acetonitrile (60 mL) was stirred for 20 hours at 50° C. and concentrated. The residue was dissolved in EA, washed with aq. NH₄C and post processed to afford 124-b (4243 mg). MS (ESI, m/z): 314[M+H]⁺.

To a solution of 124-b (4.908 g) in THF (90 mL) was added LAH (801 mg) in portions and the resulting mixture was stirred at RT for 2 hours, quenched by water (0.8 mL), aq. 15% NaOH (0.8 mL) and water (2.4 mL) in order and filtered and the filtrate was concentrated and purified to afford 124-c (2540 mg). MS (ESI, m/z): 286[M+H]⁺.

To a stirred solution of 124-c (2.44 g) in tetrahydrofuran (40 mL) was added NaH (1322 mg) under N₂ and the resulting mixture was stirred at RT for 30 min. 2-bromo-1,1-dimethoxyethane (1537 mg), DMAP (113 mg) and tetrabutylammonium iodide (639 mg) were added and the resulting mixture was stirred for 16 hours at 70° C., quenched by water and extracted with EA and post processed to afford 124-d (1.44 g). MS (ESI, m/z): 374[M+H]⁺.

To a solution of 124-d (1.44 g) in methanol (40 mL) were added Pd/C (0.50 g) and Pd(OH)₂/C (0.46 g). The reaction mixture was stirred at RT for 20 hours under H₂, filtered and the filtrate was concentrated to afford 124-e (736 mg). MS (ESI, m/z): 194[M+H]⁺.

Compound 124 (CP124, 33.5 mg, TFA salt, confirmed) was synthesized in a manner similar to CP46 with CP124-e. MS[ESI, m/z: ]: 648[M+H]⁺.

### Example 126

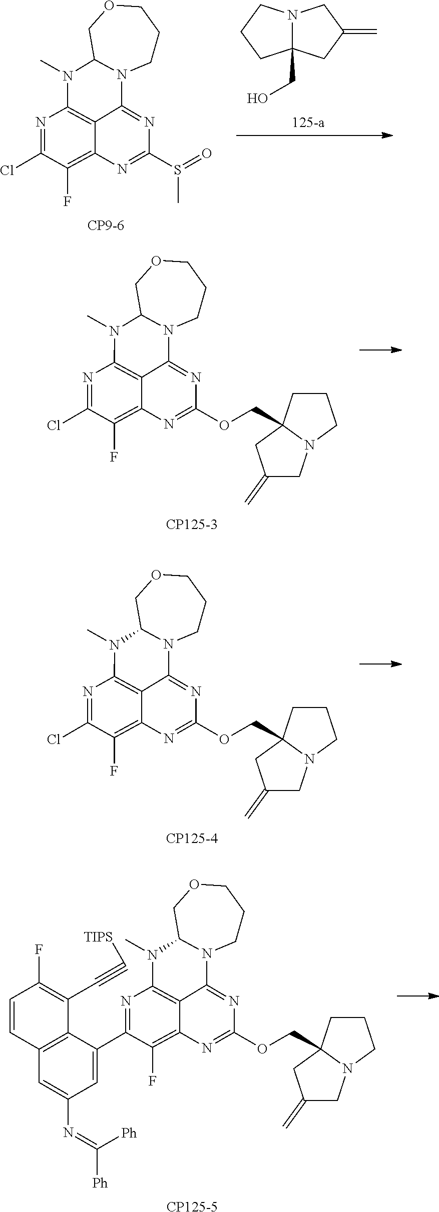

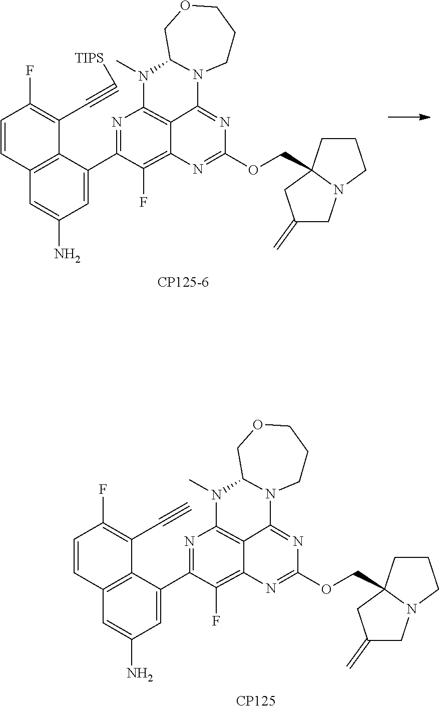

Compound 125 (CP125, confirmed) was synthesized in a manner similar to CP71 with CP125-a and CP9-6. MS: m/z: 610[M+H]⁺.

### Example 127

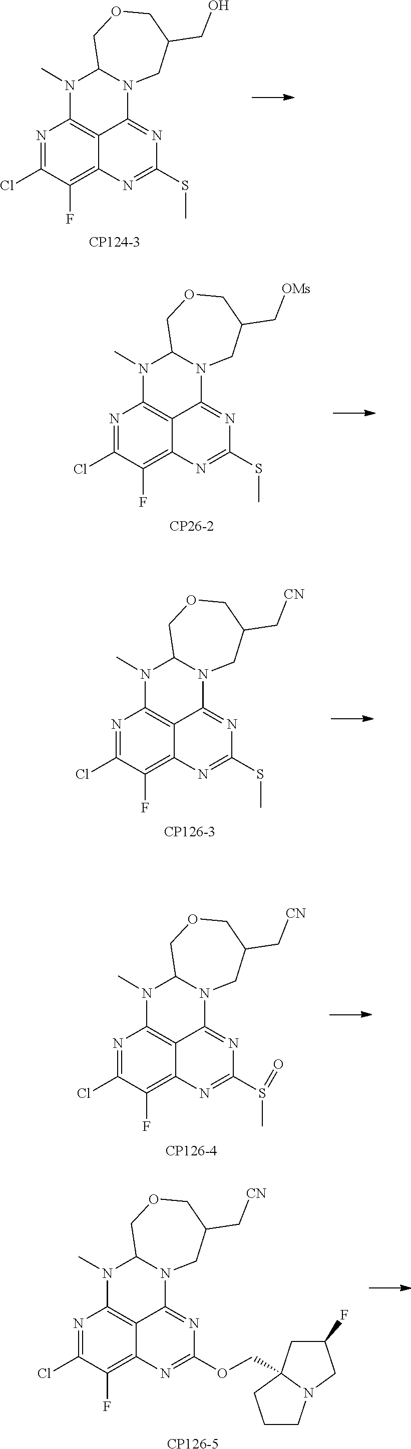

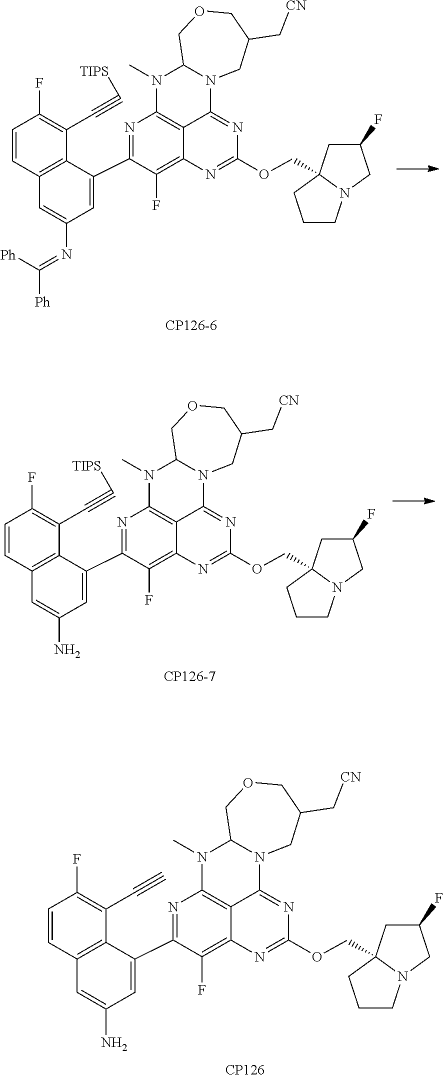

To a solution of CP124-3 (99 mg) in DCM (5 mL) were added MsCl (57 mg) and TEA (92 mg) at 0° C. The resulting solution was stirred at RT for 3 hours, diluted with DCM, washed with water and post processed to afford CP126-2 (116 mg). MS (ESI, m/z): 464[M+H]⁺.

A solution of CP126-2 (116 mg), caesium fluoride (175 mg) and cyanotrimethylsilane (97 mg) in DMF (3 mL) was stirred for 17 hours at 80° C. under N₂, diluted with EA, washed with brine and post processed to afford CP126-3 (60 mg). MS (ESI, m/z): 395[M+H]⁺.

Compound 126(CP126, 12.1 mg, TFA salt, confirmed) was synthesized in a manner similar to CP46 with CP126-3. MS (ESI, m/z): 655[M+H]⁺.

### Example 128

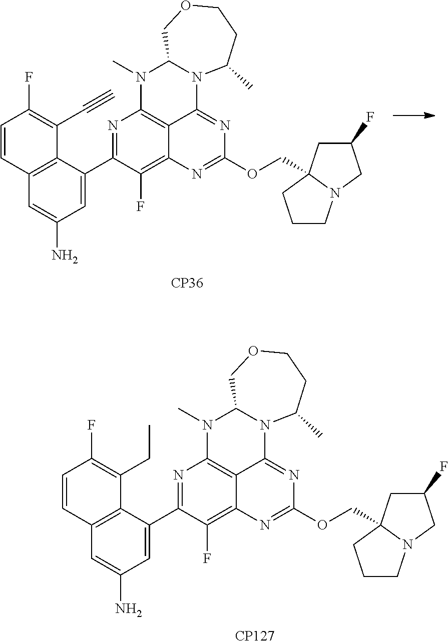

A mixture of CP36 (50 mg) and Pd/C (136 mg) in methanol (10 mL) was stirred for about 3 hours under H₂ at RT, filtered and the filtrate was concentrated and purified to afford Compound 127 (CP127, 55.6 mg, TFA salt, confirmed). MS: m/z: 634[M+1]⁺.

### Example 129

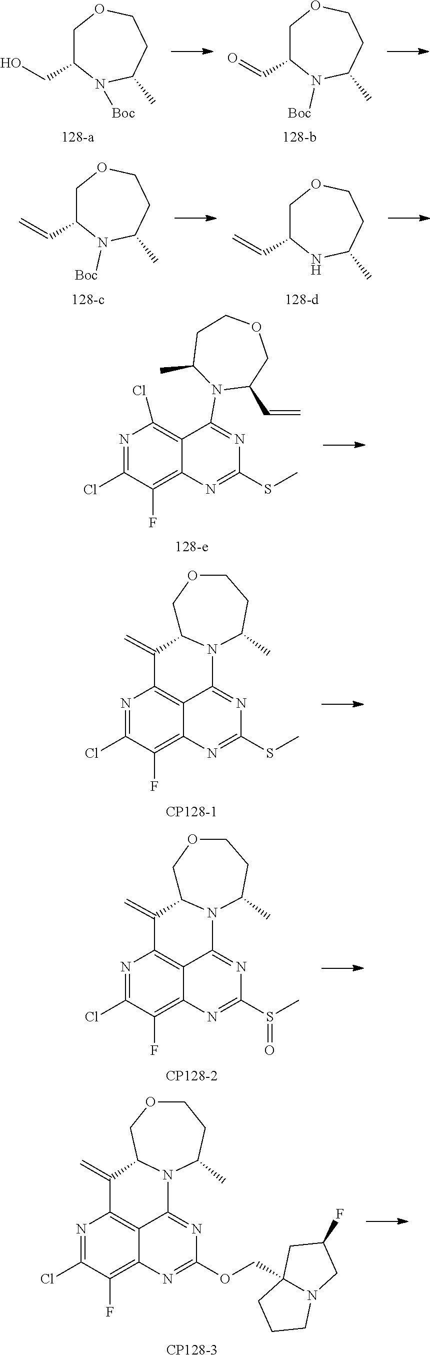

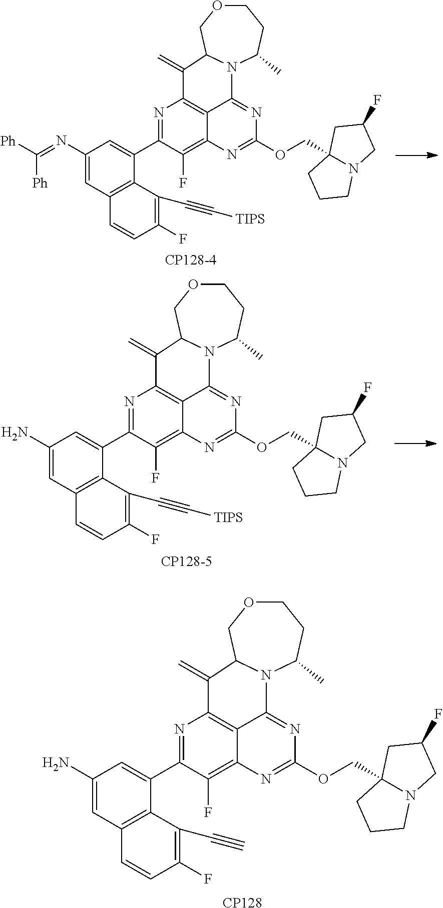

Compound 128 (CP128, 33.9 mg, TFA salt, confirmed) was synthesized in a manner similar to CP12 with CP128-a. MS: m/z: 627[M+H]⁺.

### Example 130

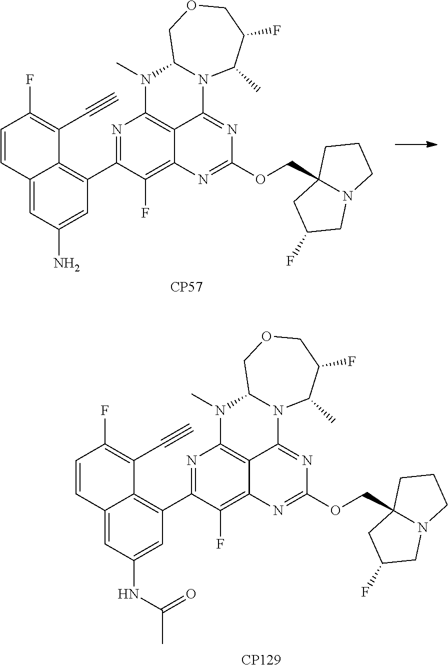

To a solution of CP57 (53 mg) and acetic anhydride (23 mg) in DCM (4 mL) was added DMAP (3 mg). The reaction was stirred at RT for 1 h under N₂, diluted with brine, extracted with DCM and post processed to afford Compound 129 (CP129, 63.3 mg, TFA salt, confirmed). MS: m/z: 690[M+H]⁺.

### Example 131

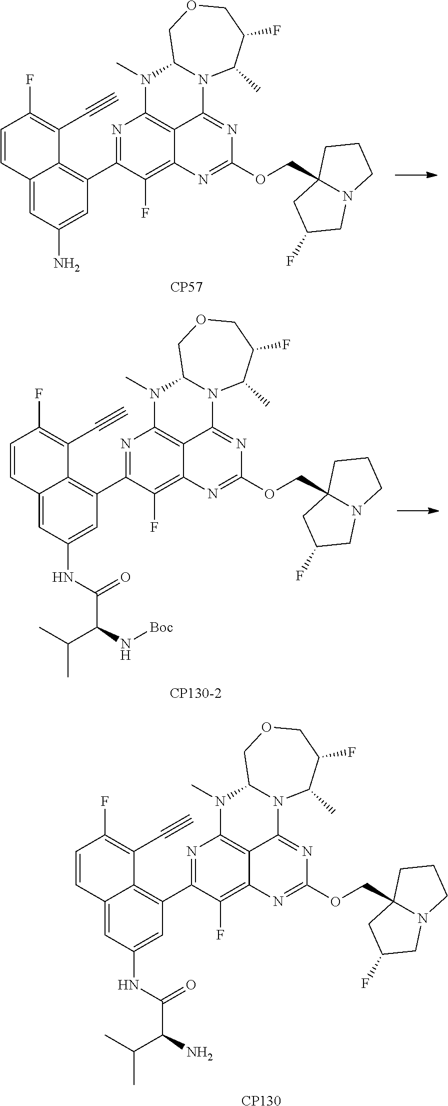

A solution of CP57 (69 mg) and N-Boc-L-valine (30 mg) in DMF (3 mL) was added HATU (111 mg) and DIEA (15 mg). The reaction was stirred at RT overnight under N₂, diluted with EA, washed with brine two times and post processed to give crude CP130-2 (126 mg). MS: m/z: 847[M+H]⁺.

A solution of CP130-2 (126 mg) and TFA (1 mL) in DCM (3 mL) was stirred at RT for 2h, quenched with sat. NaHCO₃ solution, extracted with EA and post processed to afford Compound 130 (CP130, 13.8 mg. TFA salt, confirmed). MS: m/z: 747[M+H]⁺.

The following Compounds were synthesized in a manner similar to the above-mentioned compounds:

| Compound | Structure |
|---|---|
| Compound 131 (confirmed) | 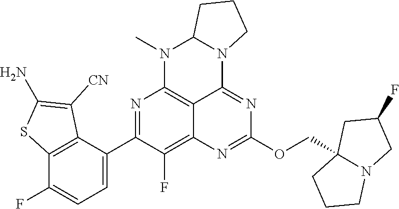 [SMILES: CN1c2nc(-c3ccc(F)c4sc(N)c(C#N)c34)c(F)c3nc(OC[C@@]45CCCN4C[C@H](F)C5)nc(c23)N2CCCC12] |
| Compound 132 (confirmed) | 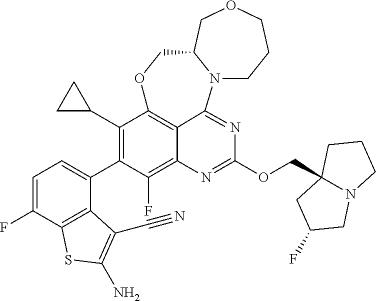 [SMILES: N#Cc1c(N)sc2c(F)ccc(-c3c(C4CC4)c4c5c(nc(OC[C@@]67CCCN6C[C@H](F)C7)nc5c3F)N3CCCOC[C@H]3CO4)c12] |
| Compound 133 (confirmed) | 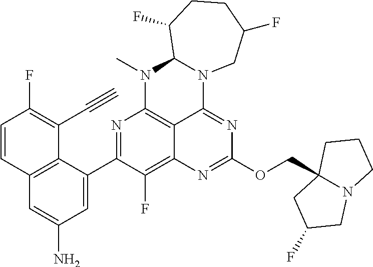 [SMILES: C#Cc1c(F)ccc2cc(N)cc(-c3nc4c5c(nc(OC[C@@]67CCCN6C[C@H](F)C7)nc5c3F)N3CC(F)CC[C@@H](F)[C@@H]3N4C)c12] |
| Compound 134 (confirmed) | 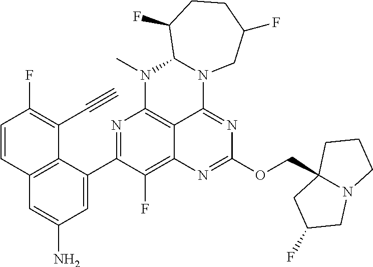 [SMILES: C#Cc1c(F)ccc2cc(N)cc(-c3nc4c5c(nc(OC[C@@]67CCCN6C[C@H](F)C7)nc5c3F)N3CC(F)CC[C@H](F)[C@H]3N4C)c12] |
| Compound 135 (assumed) | 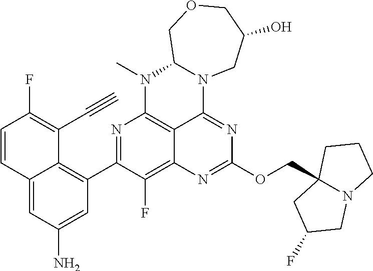 [SMILES: C#Cc1c(F)ccc2cc(N)cc(-c3nc4c5c(nc(OC[C@@]67CCCN6C[C@H](F)C7)nc5c3F)N3C[C@@H](O)COC[C@H]3N4C)c12] |
| Compound 136 (assumed) | 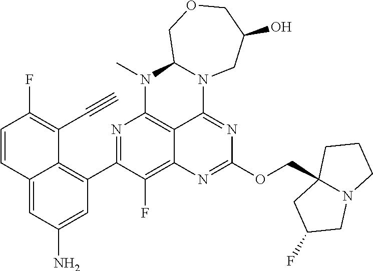 [SMILES: C#Cc1c(F)ccc2cc(N)cc(-c3nc4c5c(nc(OC[C@@]67CCCN6C[C@H](F)C7)nc5c3F)N3C[C@H](O)COC[C@@H]3N4C)c12] |
| Compound 137 (assumed) | 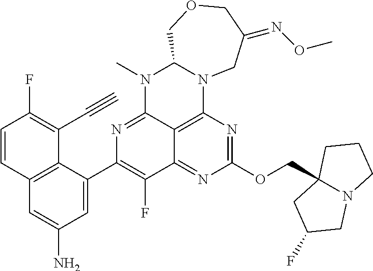 [SMILES: C#Cc1c(F)ccc2cc(N)cc(-c3nc4c5c(nc(OC[C@@]67CCCN6C[C@H](F)C7)nc5c3F)N3C/C(=N\OC)COC[C@H]3N4C)c12] |
| Compound 138 (confirmed) | 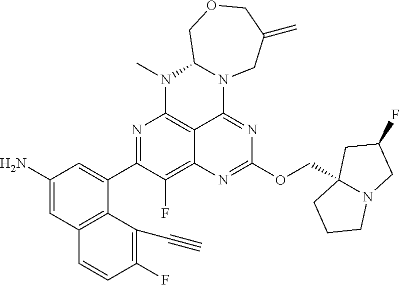 [SMILES: C#Cc1c(F)ccc2cc(N)cc(-c3nc4c5c(nc(OC[C@@]67CCCN6C[C@H](F)C7)nc5c3F)N3CC(=C)COC[C@H]3N4C)c12] |
| Compound 139 (confirmed) | 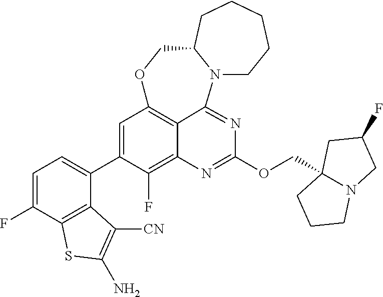 [SMILES: N#Cc1c(N)sc2c(F)ccc(-c3cc4c5c(nc(OC[C@@]67CCCN6C[C@H](F)C7)nc5c3F)N3CCCCC[C@H]3CO4)c12] |
| Compound 140 (confirmed) | 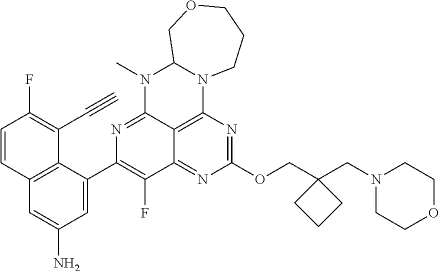 [SMILES: C#Cc1c(F)ccc2cc(N)cc(-c3nc4c5c(nc(OCC6(CN7CCOCC7)CCC6)nc5c3F)N3CCCOCC3N4C)c12] |
| Compound 141 (confirmed) | 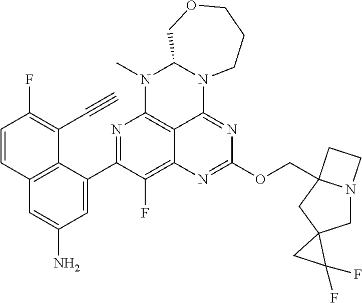 [SMILES: C#Cc1c(F)ccc2cc(N)cc(-c3nc4c5c(nc(OCC67CCN6CC6(C7)CC6(F)F)nc5c3F)N3CCCOC[C@H]3N4C)c12] |
| Compound 142 (confirmed) | 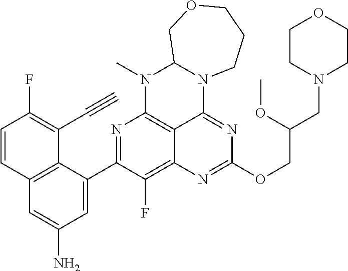 [SMILES: C#Cc1c(F)ccc2cc(N)cc(-c3nc4c5c(nc(OCC(CN6CCOCC6)OC)nc5c3F)N3CCCOCC3N4C)c12] |
| Compound 143 (confirmed) | 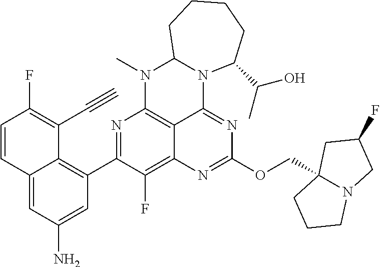 [SMILES: C#Cc1c(F)ccc2cc(N)cc(-c3nc4c5c(nc(OC[C@@]67CCCN6C[C@H](F)C7)nc5c3F)N3C(CCCC[C@@H]3C(C)O)N4C)c12] |
| Compound 144 (confirmed) | 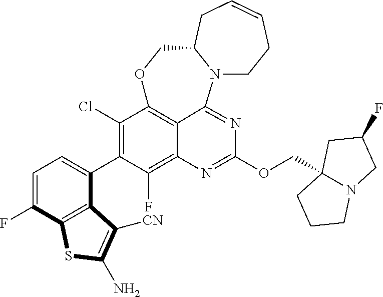 |
| Compound 145 (confirmed) | 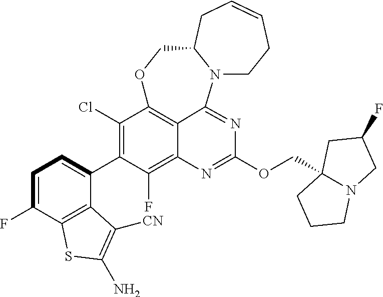 |
| Compound 146 (assumed) | 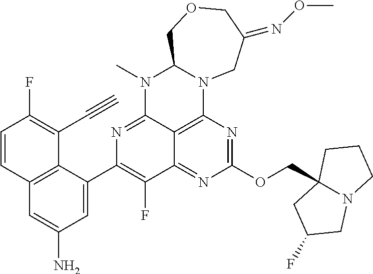 [SMILES: C#Cc1c(F)ccc2cc(N)cc(-c3nc4c5c(nc(OC[C@@]67CCCN6C[C@H](F)C7)nc5c3F)N3C/C(=N/OC)COC[C@@H]3N4C)c12] |
| Compound 147 (assumed) | 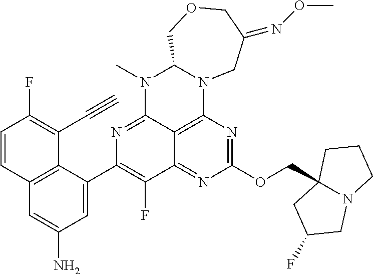 [SMILES: C#Cc1c(F)ccc2cc(N)cc(-c3nc4c5c(nc(OC[C@@]67CCCN6C[C@H](F)C7)nc5c3F)N3C/C(=N/OC)COC[C@H]3N4C)c12] |
| Compound 148 (assumed) | 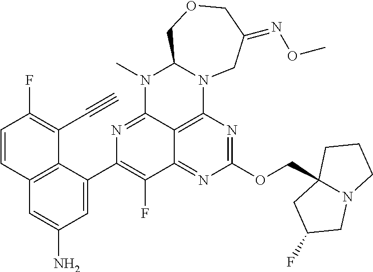 [SMILES: C#Cc1c(F)ccc2cc(N)cc(-c3nc4c5c(nc(OC[C@@]67CCCN6C[C@H](F)C7)nc5c3F)N3C/C(=N\OC)COC[C@@H]3N4C)c12] |
| Compound 149 | 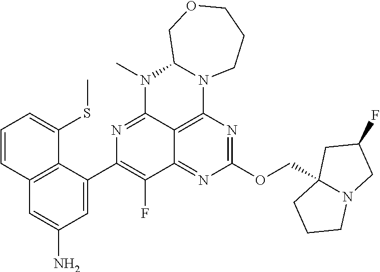 [SMILES: CSc1cccc2cc(N)cc(-c3nc4c5c(nc(OC[C@@]67CCCN6C[C@H](F)C7)nc5c3F)N3CCCOC[C@H]3N4C)c12] |
| | (confirmed) |

### Example 150

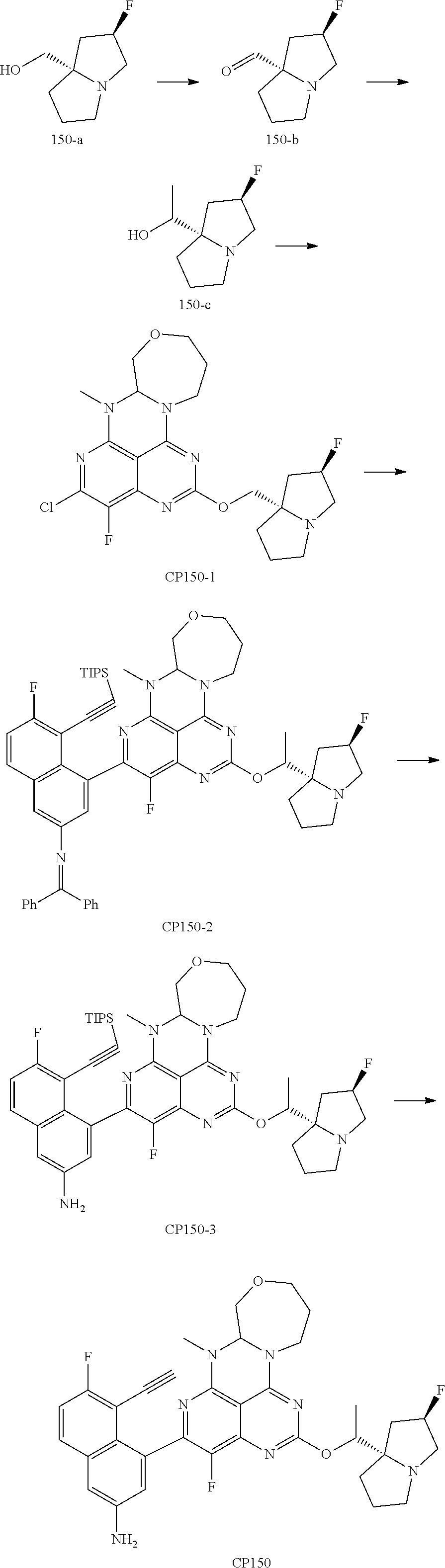

Compound 150 (CP150, confirmed) was synthesized in a manner similar to CP46. MS: m/z: 630[M+1]⁺.

### Example 151

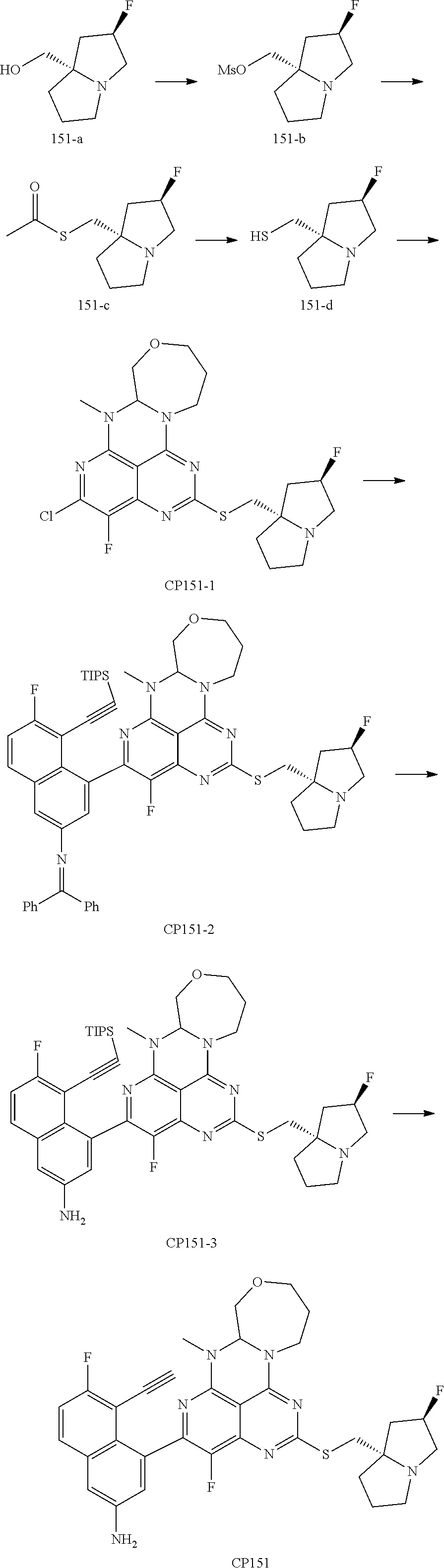

Compound 151 (CP151, confirmed) was synthesized in a manner similar to CP46. MS: m/z: 632[M+1]⁺.

### Example 152

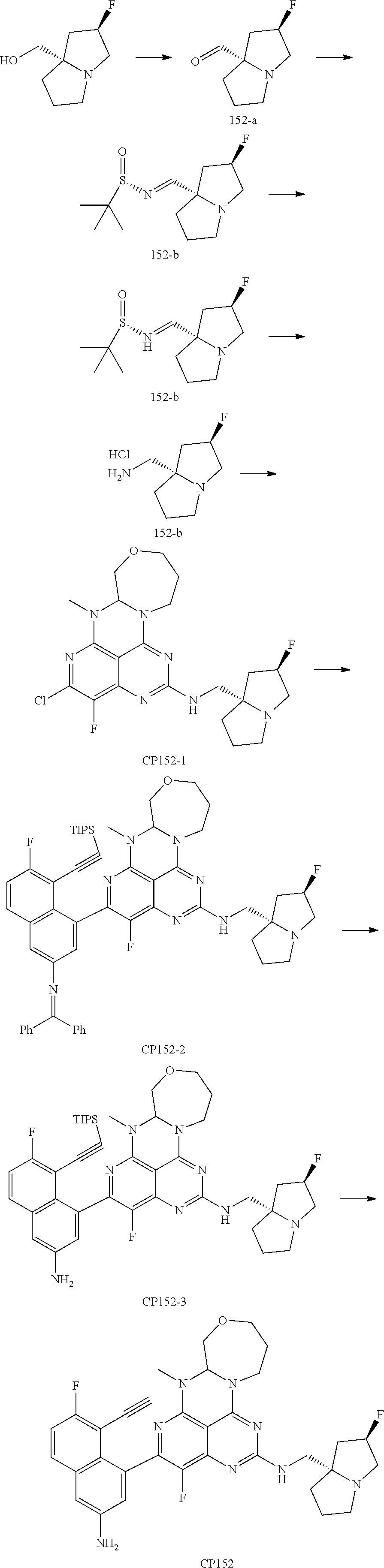

Compound 152 (CP152, confirmed) was synthesized in a manner similar to CP46. MS: m/z: 615[M+1]⁺.

### Example 153

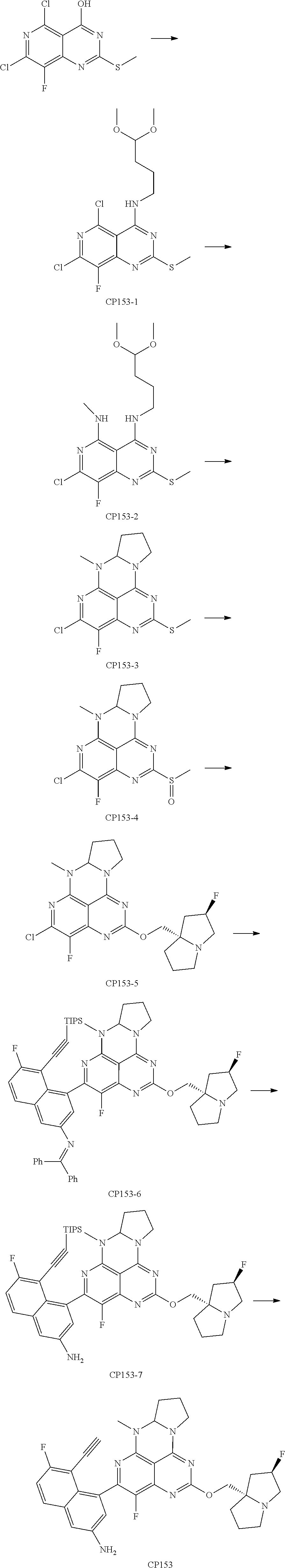

Compound 153 (CP153, confirmed) was synthesized in a manner similar to CP46. MS: m/z: 586 [M+H]⁺, ¹HNMR (400 MHz, DMSO-d₆) δ=7.74 (dd, J=9.2, 5.9 Hz, 1H), 7.31 (t, J=9.0 Hz, 1H), 7.03-6.99 (m, 2H), 5.59 (s, 2H), 5.34 (s, 0.5H), 5.21 (s, 0.5H), 4.97-4.91 (m, 1H), 4.13-3.88 (m, 3H), 3.81-3.61 (m, 2H), 3.16-2.97 (m, 3H), 2.89 (d, J=4.6 Hz, 3H), 2.86-2.77 (m, 1H), 2.25-1.93 (m, 7H), 1.90-1.68 (m, 3H).

### Example 154

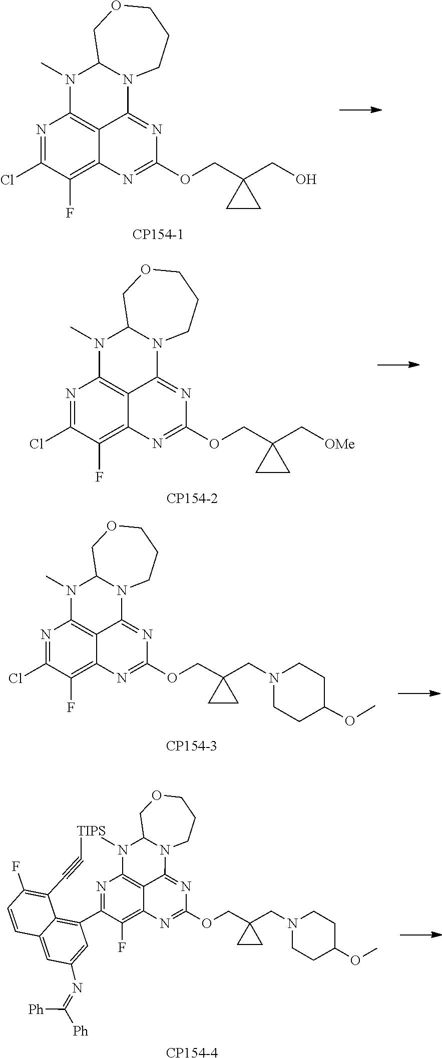

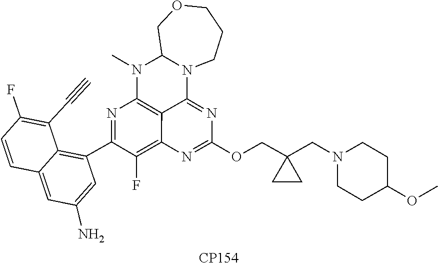

Compound 154 (CP154, confirmed) was synthesized in a manner similar to CP46. MS: m/z: 507 [M+H]⁺.

### Example 155

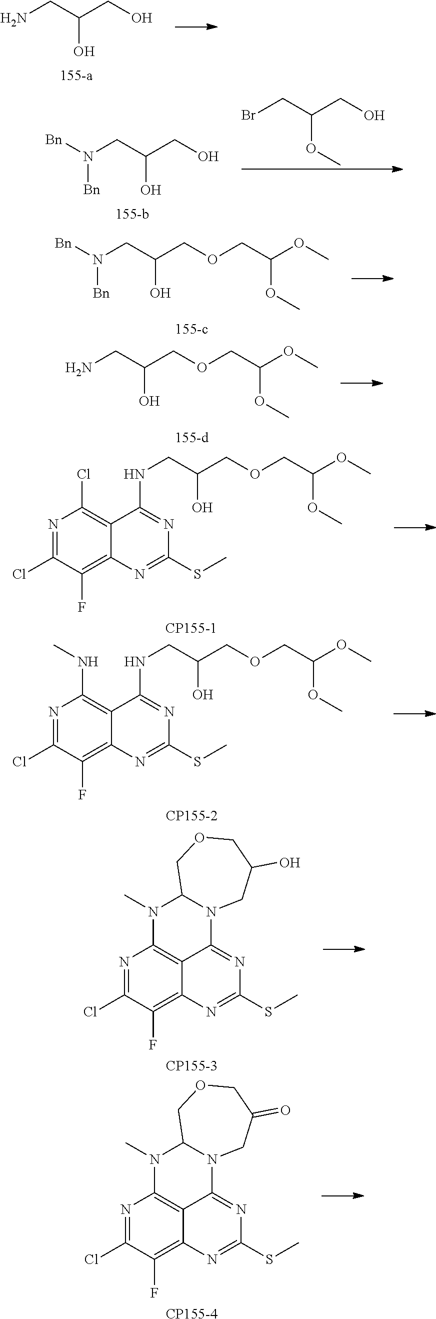

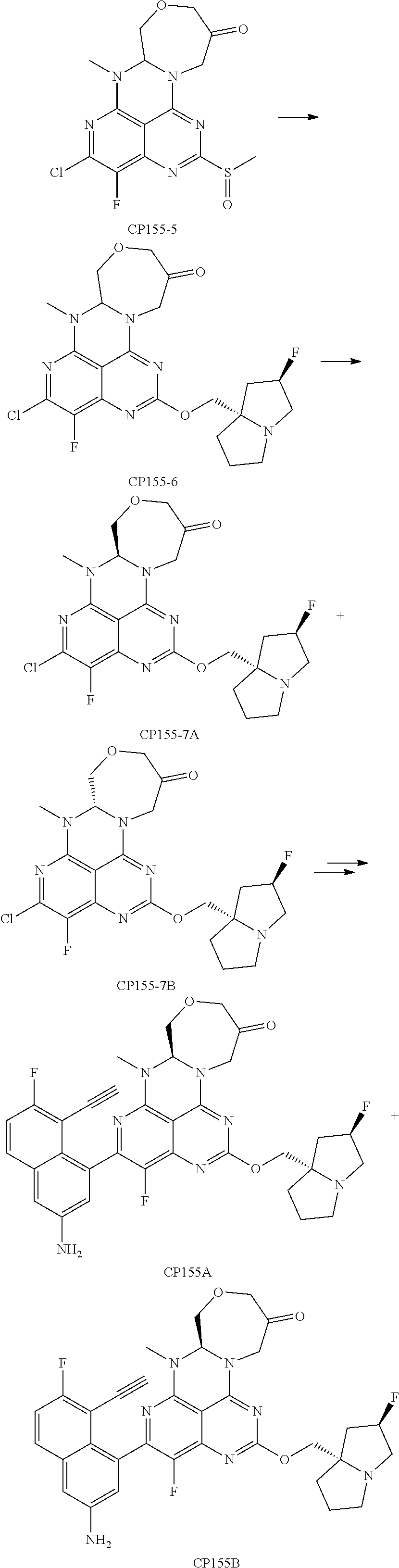

CP155-6 (57 mg) was separated by Prep-HPLC-Gilson with the conditions: Column, CHIRAL ART Cellulose-SC column (2 cm×25 cm, 5 μm); mobile phase, (Hex:DCM=3:1) (0.1% DEA)/EtOH (50:50); Flowing rate: 15 mL/min. This resulted in CP155-7A (19 mg, the first eluting isomer, Retention Time: 7.073 min) and CP155-7B (19 mg, the second eluting isomer, Retention Time: 11.157 min).

Compound 155A (CP155A, confirmed, MS m/z: 630[M+H]⁺) and Compound 155B (CP155B, confirmed, MS m/z: 630[M+H]⁺) were synthesized in a manner similar to CP46.

### Example 156

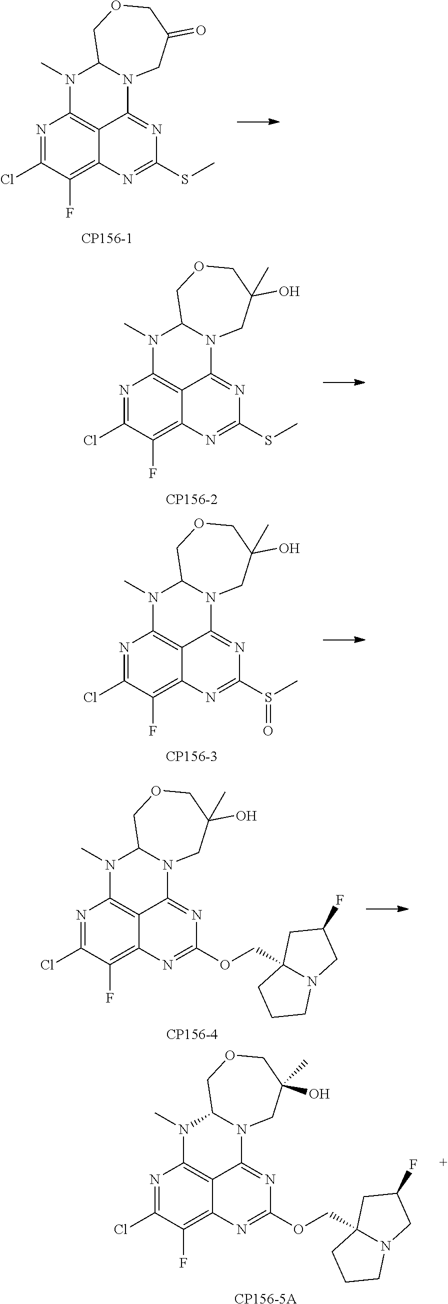

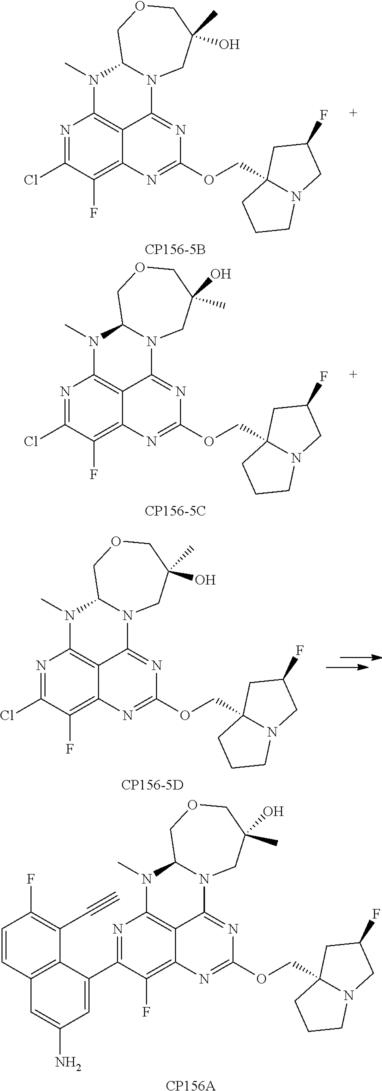

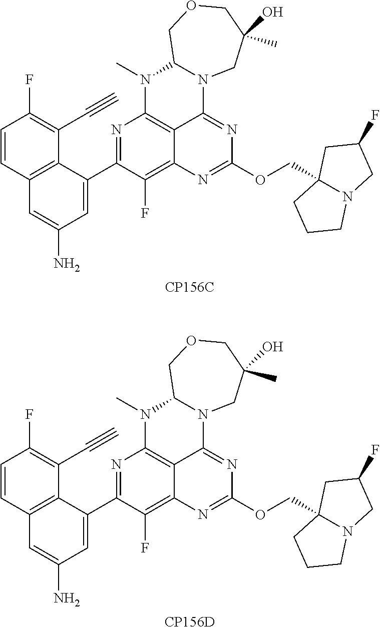

CP156-4 was separated by Prep-HPLC-Gilson with the conditions: Column, CHIRAL ART Cellulose-SC column (2 cm×25 cm, 5 um); mobile phase, (Hex:DCM=2:1)(0.1% DEA)/EtOH (50:50); Flowing rate: 15 mL/min. This resulted in CP156-5A (32 mg, the first eluting isomer, Retention Time: 4.877 min) and CP156-5B (37 mg, the second eluting isomer, Retention Time: 5.813 min).

The intermediate CP156-4 (another peak) was separated by Prep-HPLC-Gilson with the conditions: Column, CHIRAL ART Cellulose-SC column (2 cm×25 cm, 5 um); mobile phase, (Hex:DCM=2:1) (0.1% DEA)/EtOH (50:50); Flowing rate: 15 mL/min. This resulted in CP156-SC (26 mg, the first eluting isomer, Retention Time: 5.193 min) and CP156-5D (27 mg, the second eluting isomer, Retention Time: 9.827 min).

Compound 156A (CP156A, assumed, MS m/z: 646[M+H]⁺), Compound 156B (CP156B, assumed, MS m/z: 646[M+H]⁺), Compound 156C (CP156C, assumed, MS m/z: 646[M+H]⁺) and Compound 156D (CP156D, assumed, MS m/z: 646[M+H]⁺) were synthesized in a manner similar to CP46.

### Example 157

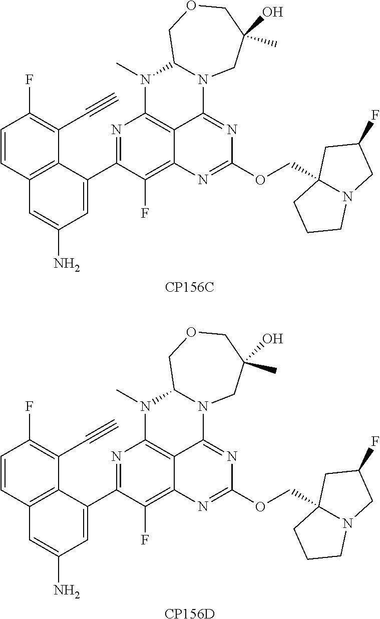

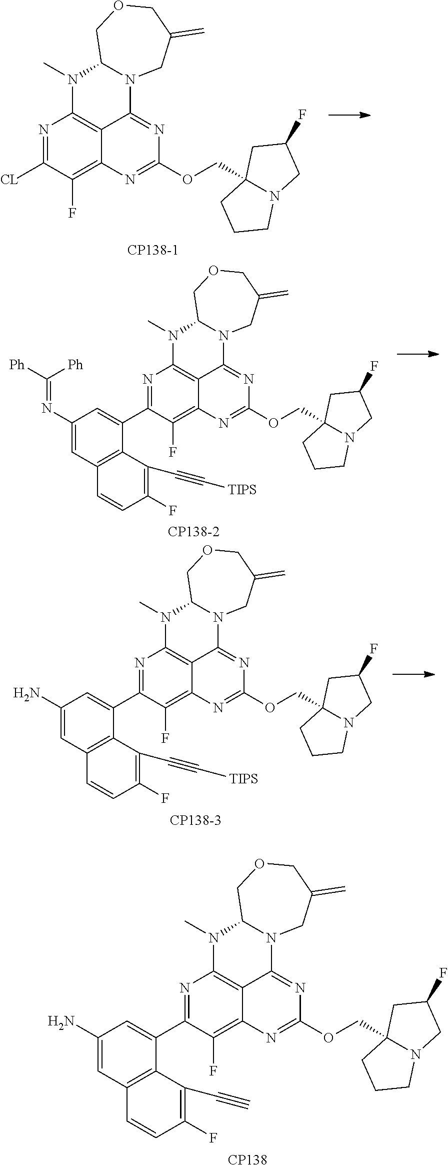

CP157-4 was separated by Prep-HPLC-Gilson with the conditions: Column, CHIRAL ART Cellulose-SC column (2 cm×25 cm, 5 um); mobile phase (Hex:DcM=3:1)(0.1% DEA)/EtOH (50:50); Flowing rate: 15 mL/min. This resulted in CP157-5A (38 mg, the first eluting isomer, Retention Time: 7.631 min) and CP157-5B (39 mg, the second eluting isomer, Retention Time: 9.974 min).

Compound 157A (CP157A, confirmed. MS m/z: 628[M+H]⁺) was synthesized in a manner similar to CP46.

Compound 138 (CP138, confirmed. MS m/z: 628[M+H]⁺) was synthesized in a manner similar to CP46.

### Example 158

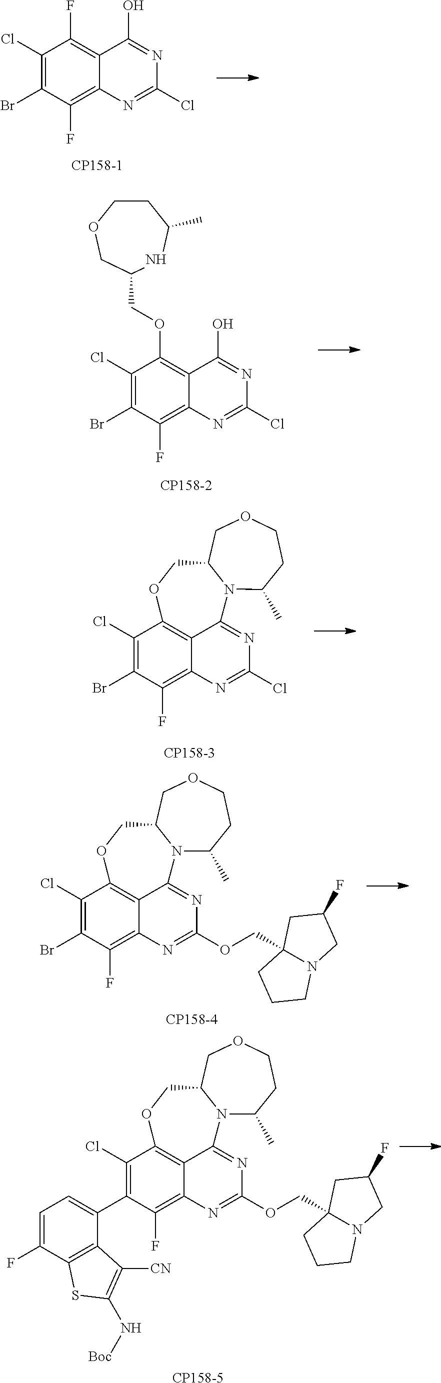

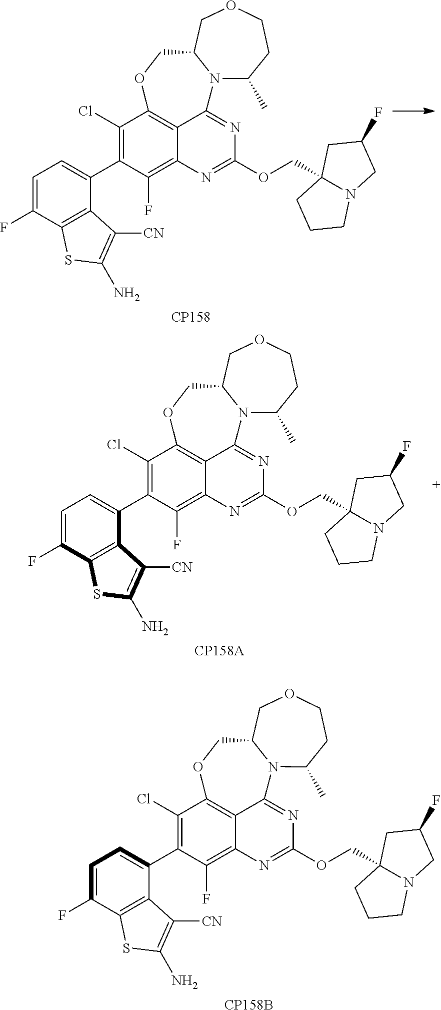

Compound 158 (CP158, confirmed, MS m/z: 671[M+H]⁺) was synthesized in a manner similar to CP1 and separated by Prep-HPLC-Gilson with the following conditions: Column, CHIRAL ART Cellulose-SB column (2 cm×25 cm, 5 um): mobile phase, (Hex:DcM=3:1)(0.1% DEA)/EtOH (50:50); Flowing rate: 20 ml/min. This resulted in Compound 158A (the first eluting isomer, Retention Time: 3.705 min) and Compound 158B (the second eluting isomer, Retention Time: 4.773 min)

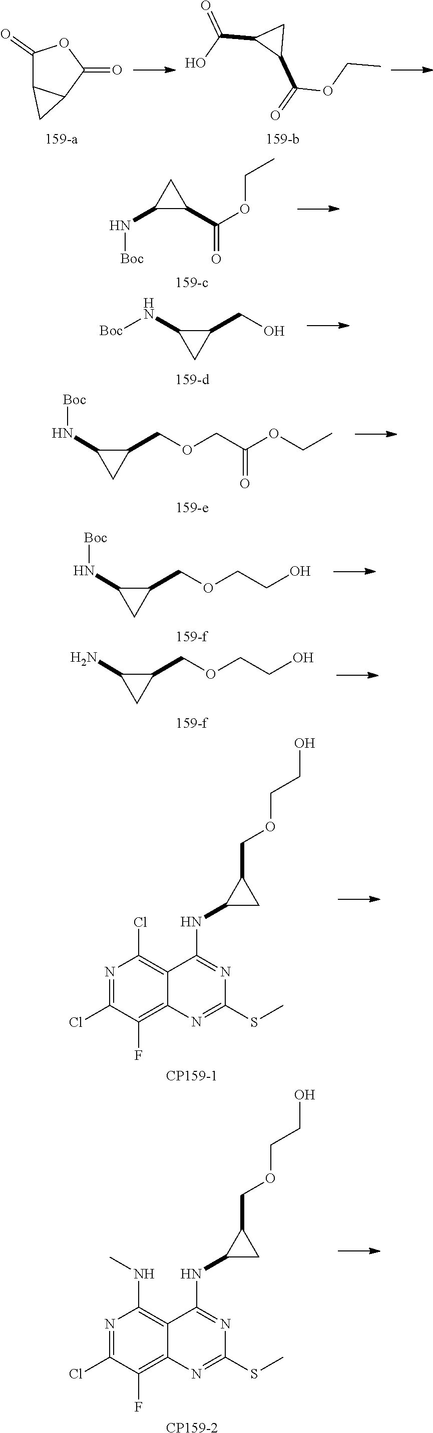

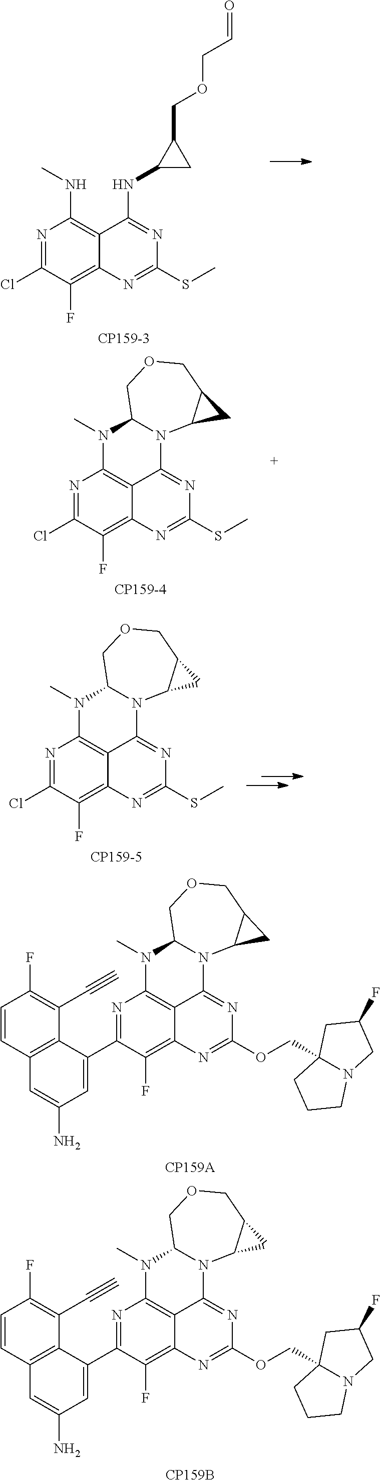

Compound 159A (CP159A, assumed. MS m/z: 628[M+H]⁺), Compound 159B (CP159B, assumed. MS m/z: 628[M+H]⁺) were synthesized in a manner similar to CP46.

### Example 160

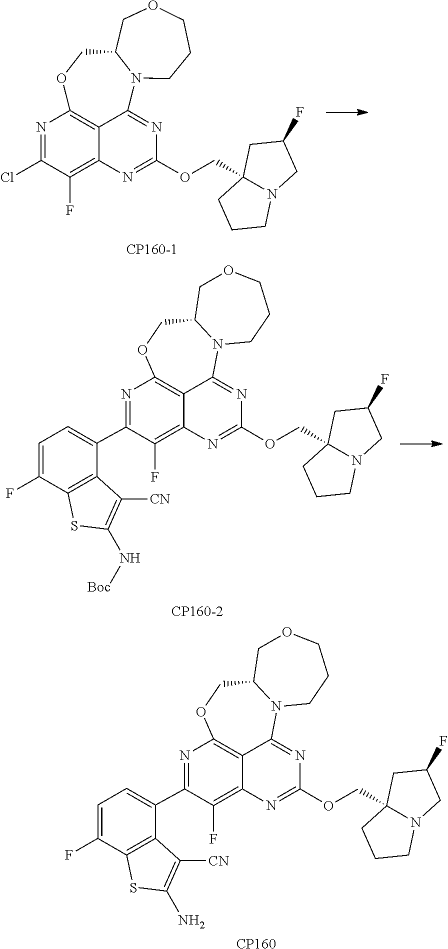

Compound 160 (CP160, confirmed, MS m/z: 624[M+H]⁺) was synthesized in a manner similar to CP1.

### Example 161

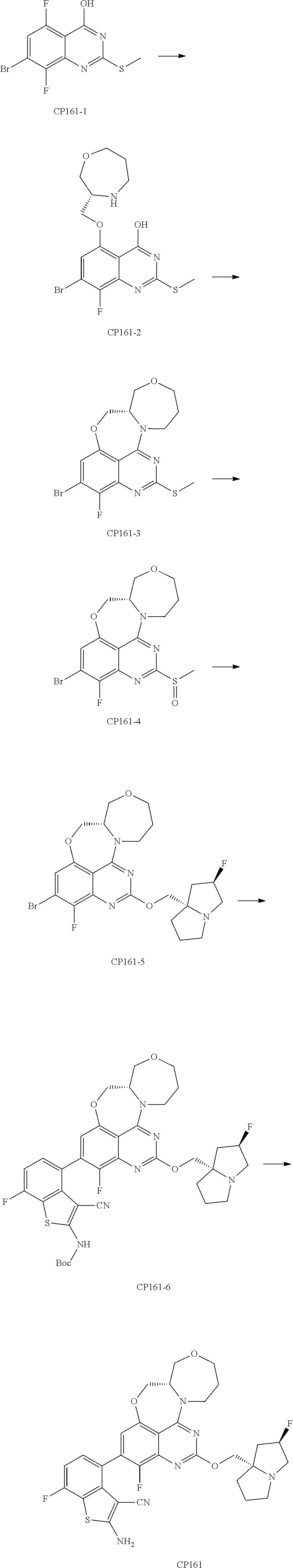

Compound 161 (CP161, confirmed, MS m/z: 623[M+H]⁺) was synthesized in a manner similar to CP11.

### Example 162

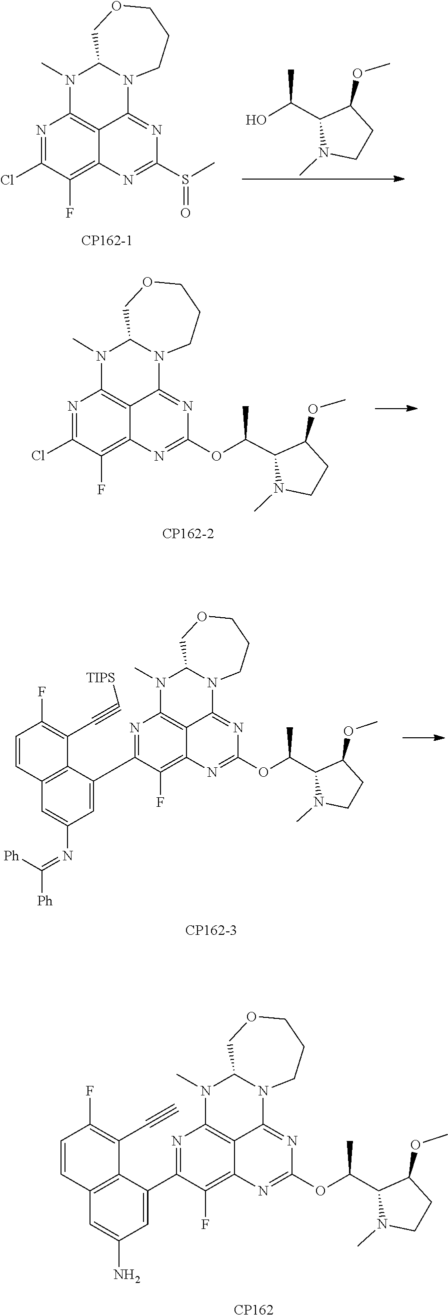

Compound 162 (CP162, confirmed, MS m/z: 616 [M+H]⁺) was synthesized in a manner similar to CP46.

### Example 163

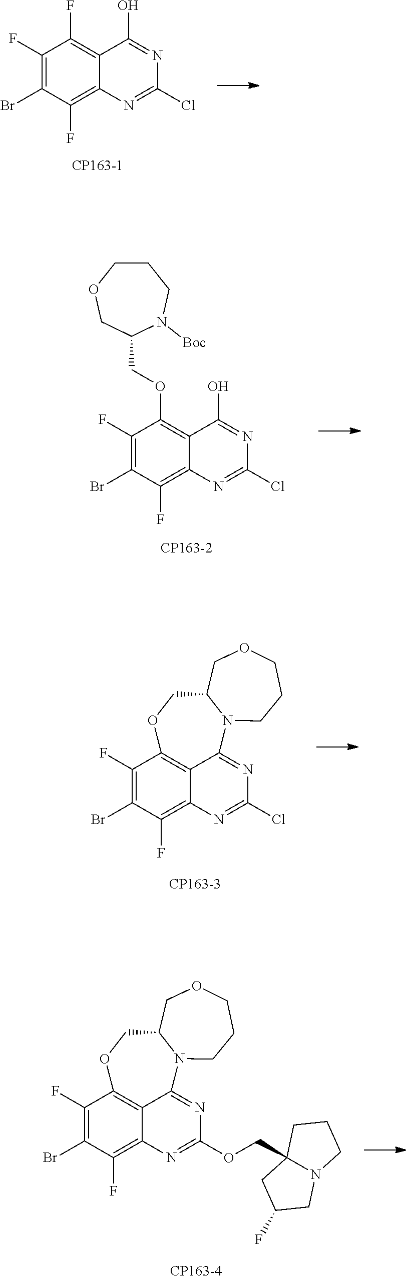

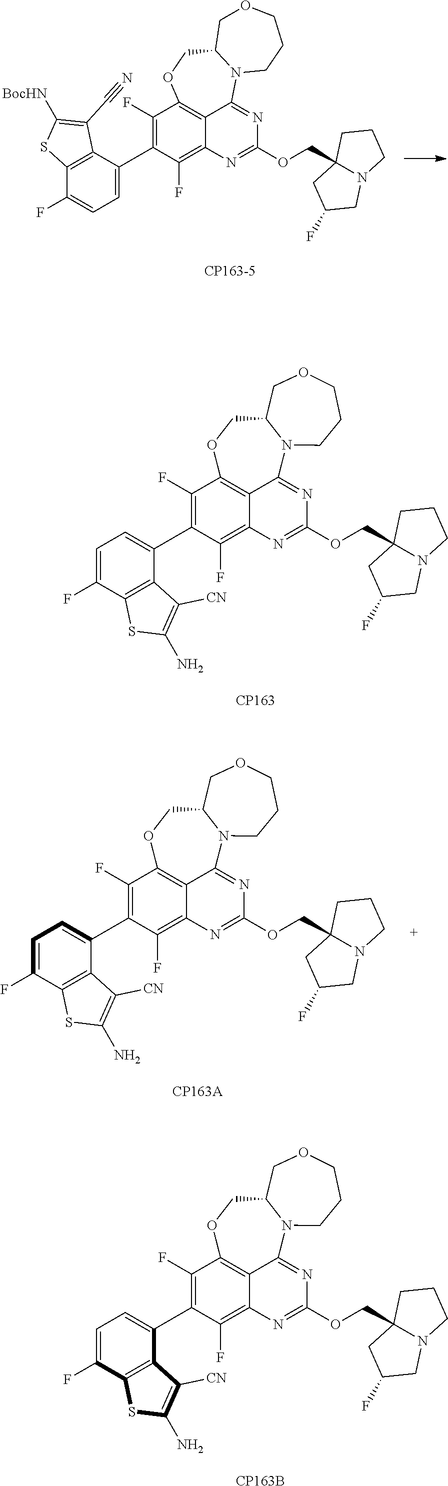

Compound 163 (CP163) were synthesized in a manner similar to CP66 and separated by the conditions: mobile Column, CHIRAL ART Amylose-SA column (2 cm×25 cm, 5 μm); phase, (Hex:DCM=3:1) (0.1% DEA)/EtOH (50:50); Flowing rate: 20 mL/min. This resulted in Compound 163A (CP163A, confirmed, the first eluting isomer, Retention Time: 3.877 min) and Compound 163B (CP163B, confirmed, the second eluting isomer, Retention Time: 5.047 min). MS: m/z: 641[M+H]⁺.

### Example 164

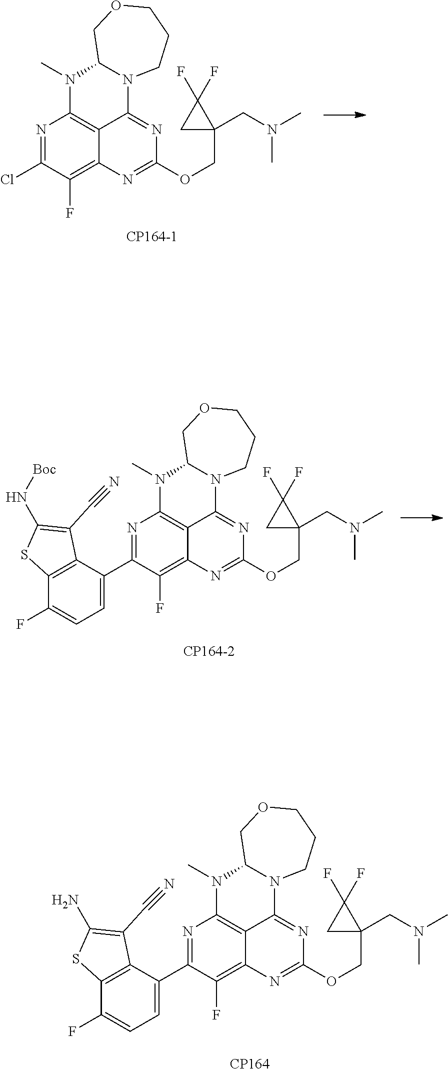 [SMILES: "CP164-1" ; "CP164" ; "CP164-2" ; CN(C)CC1(COc2nc3c4c(nc(Cl)c(F)c4n2)N(C)[C@@H]2COCCCN32)CC1(F)F ; CN(C)CC1(COc2nc3c4c(nc(-c5ccc(F)c6sc(NC(=O)OC(C)(C)C)c(C#N)c56)c(F)c4n2)N(C)[C@@H]2COCCCN32)CC1(F)F ; CN(C)CC1(COc2nc3c4c(nc(-c5ccc(F)c6sc(N)c(C#N)c56)c(F)c4n2)N(C)[C@@H]2COCCCN32)CC1(F)F]

Compound 164 (CP164, confirmed. MS m/z: 629[M+H]⁺) was synthesized in a manner similar to CP46.

### Example 165

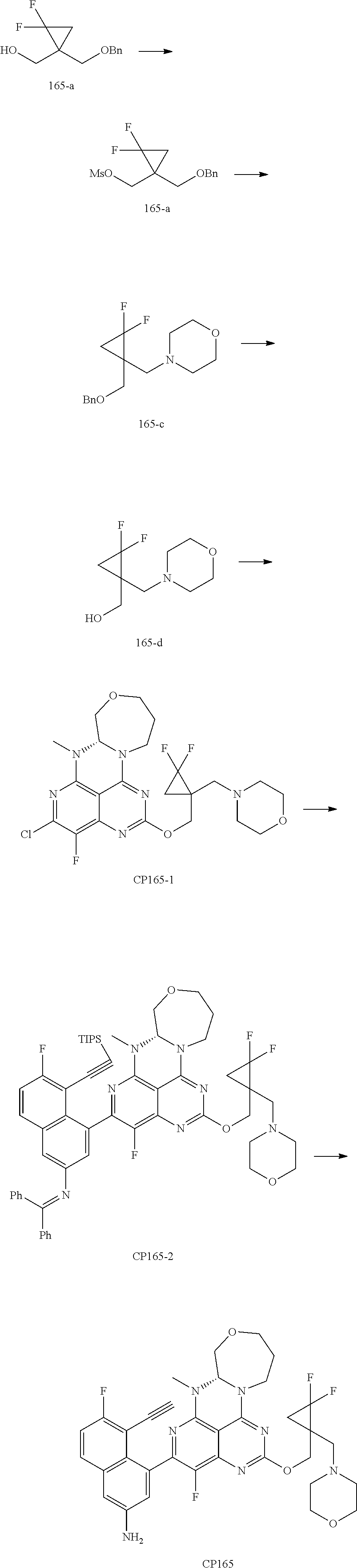

Compound 165 (CP165, confirmed, MS: m/z: 664[M+H]⁺) was synthesized in a manner similar to CP46.

### Example 166

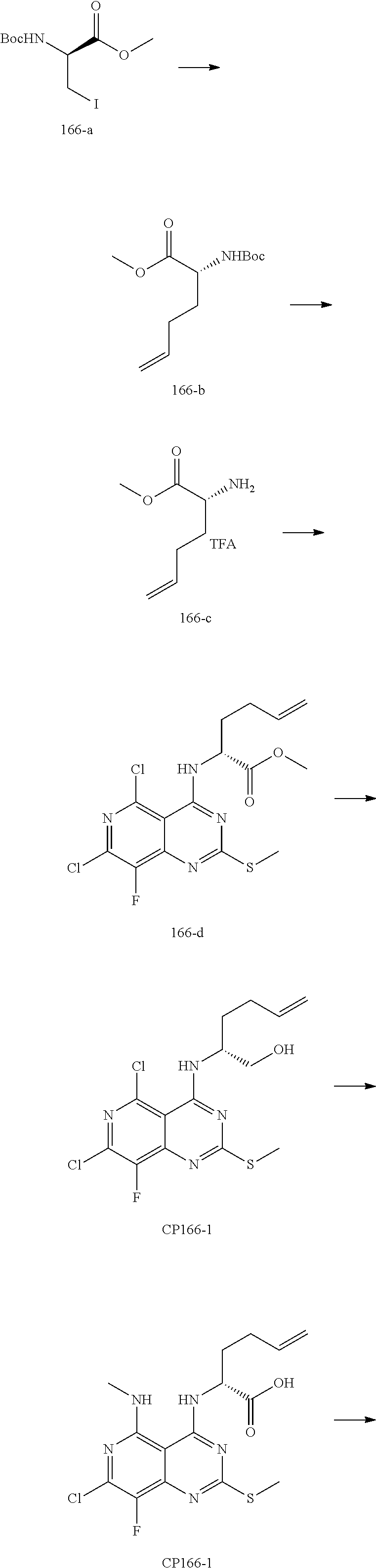

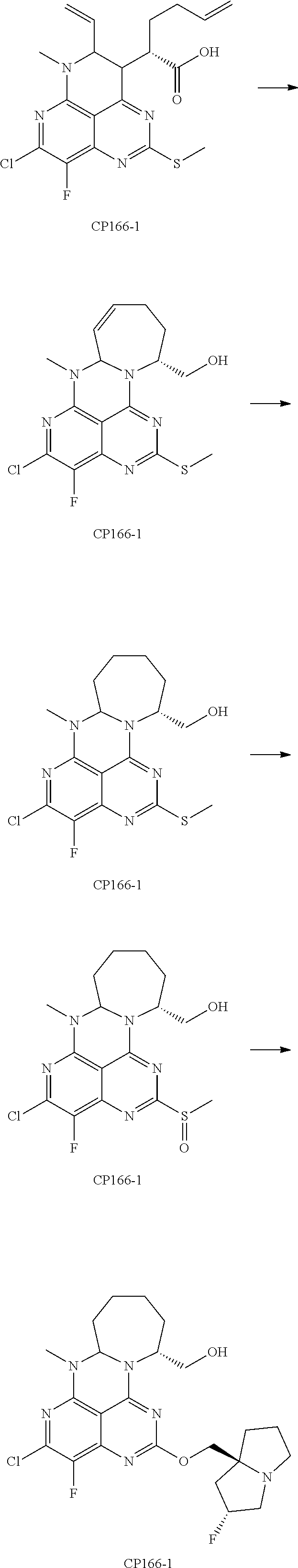

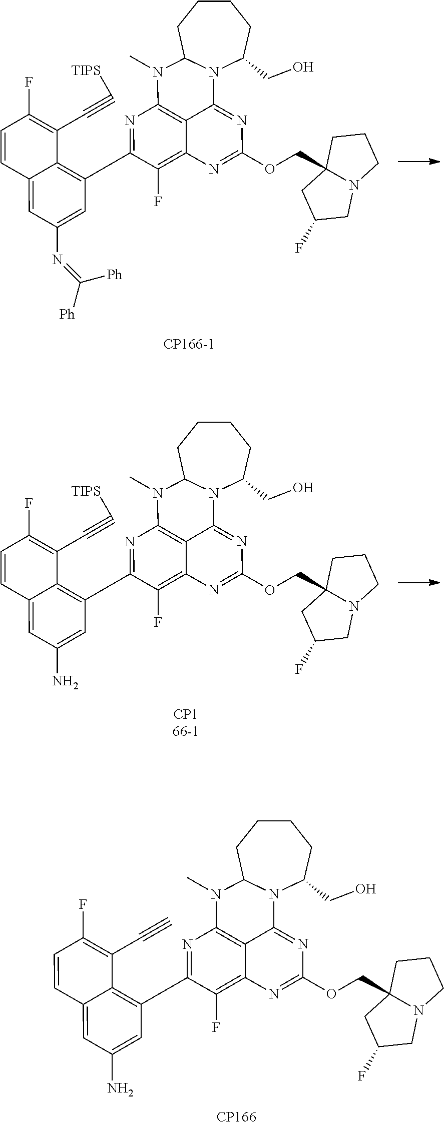

Compound 166 (CP166, confirmed. MS m/z: 639[M+H]⁺) was synthesized in a manner similar to CP46.

### Example 167

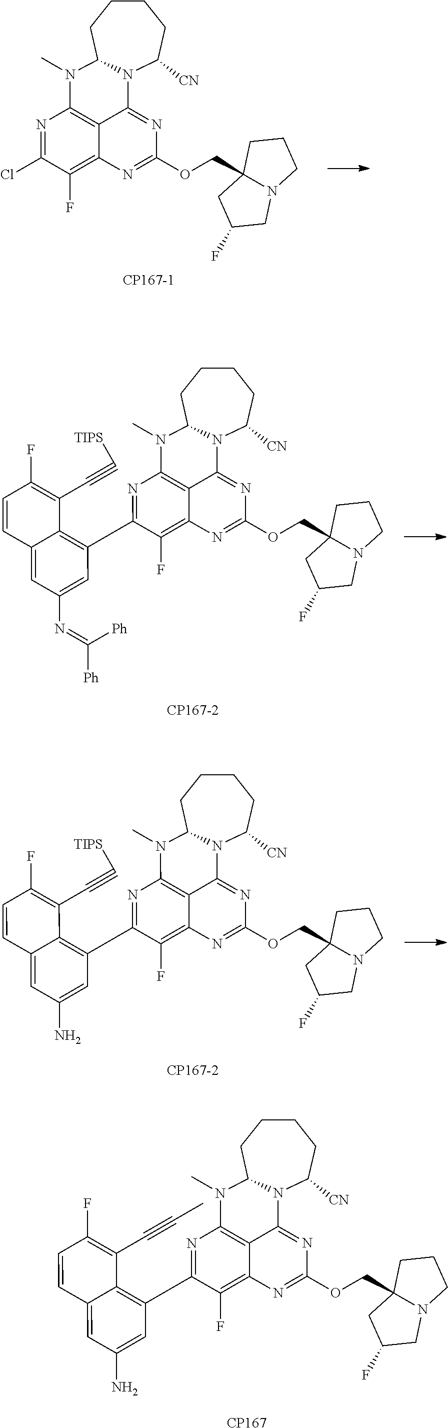

Compound 167 (CP167, confirmed, MS: m/z: 644[M+H]⁺) was synthesized in a manner similar to CP46.

### Example 168

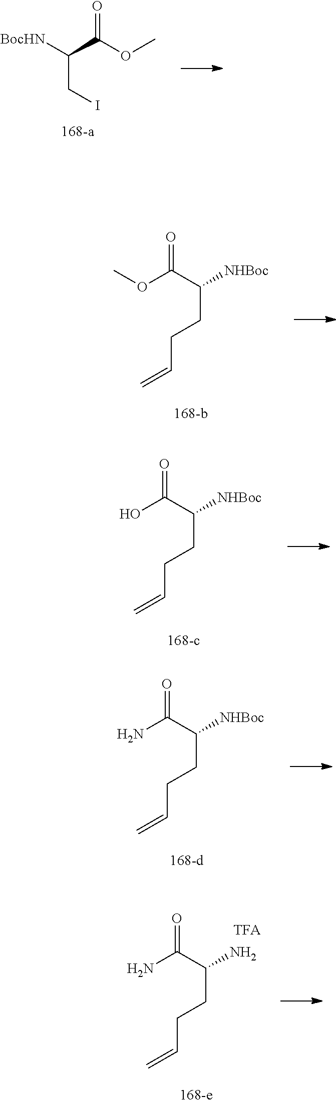

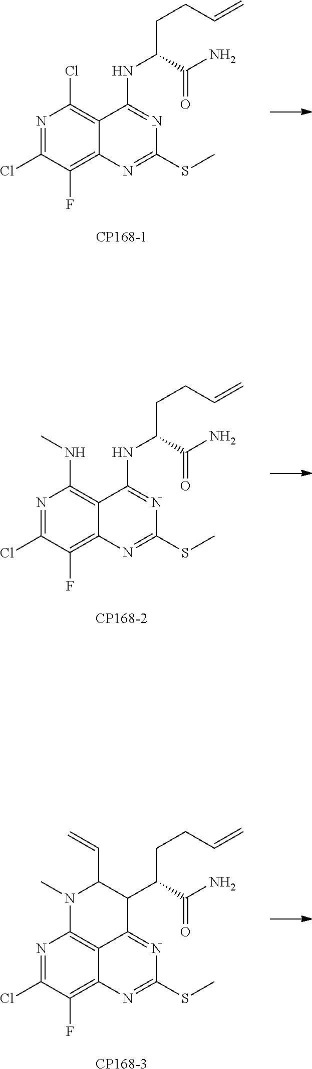 [SMILES: "CP168-2" ; "CP168-1" ; "CP168-3" ; C=CCC[C@@H](Nc1nc(SC)nc2c(F)c(Cl)nc(NC)c12)C(N)=O ; C=CCC[C@H](C(N)=O)C1c2nc(SC)nc3c(F)c(Cl)nc(c23)N(C)C1C=C ; C=CCC[C@@H](Nc1nc(SC)nc2c(F)c(Cl)nc(Cl)c12)C(N)=O]

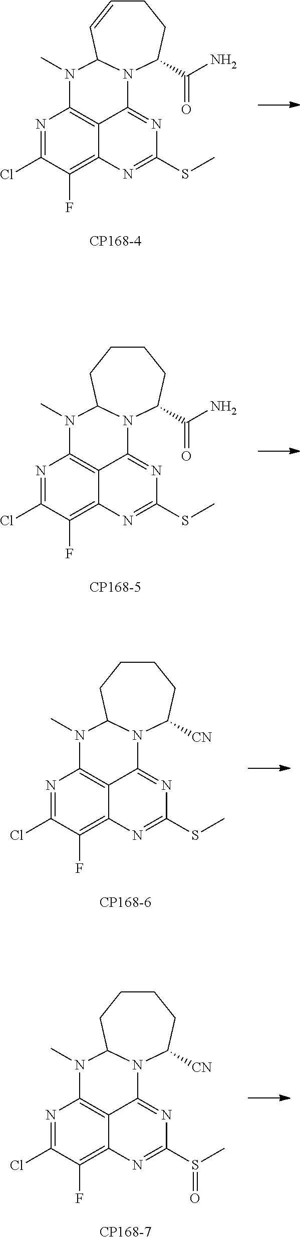

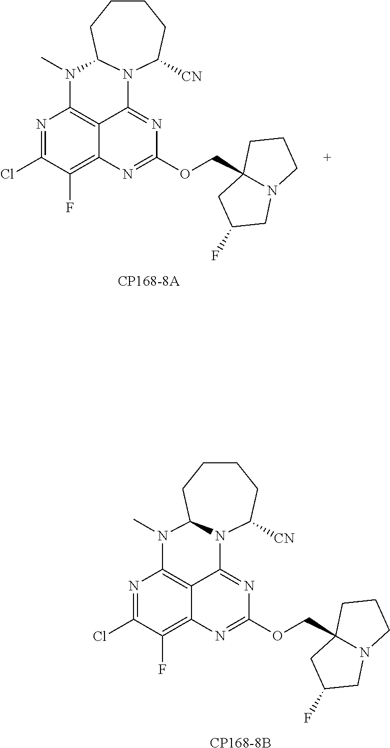 [SMILES: "CP168-8A" ; "CP168-8B" ; CN1c2nc(Cl)c(F)c3nc(OC[C@@]45CCCN4C[C@H](F)C5)nc(c23)N2[C@@H](C#N)CCCC[C@@H]12 ; CN1c2nc(Cl)c(F)c3nc(OC[C@@]45CCCN4C[C@H](F)C5)nc(c23)N2[C@@H]1CCCC[C@@H]2C#N]

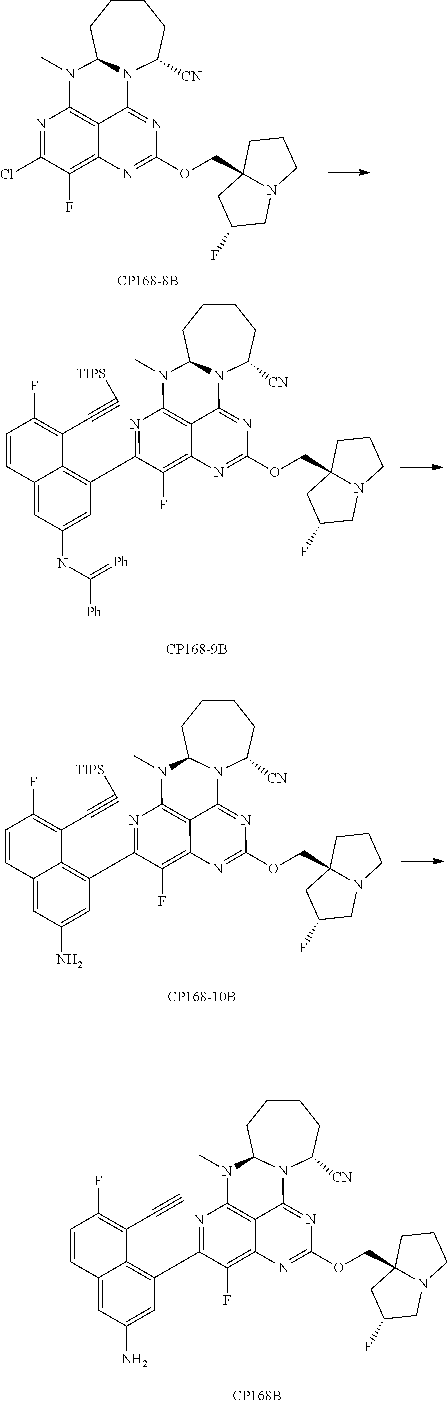

Compound 168B (CP168B, confirmed. MS: m/z: 639[M+H]⁺) was synthesized in a manner similar to CP46.

### Example 169

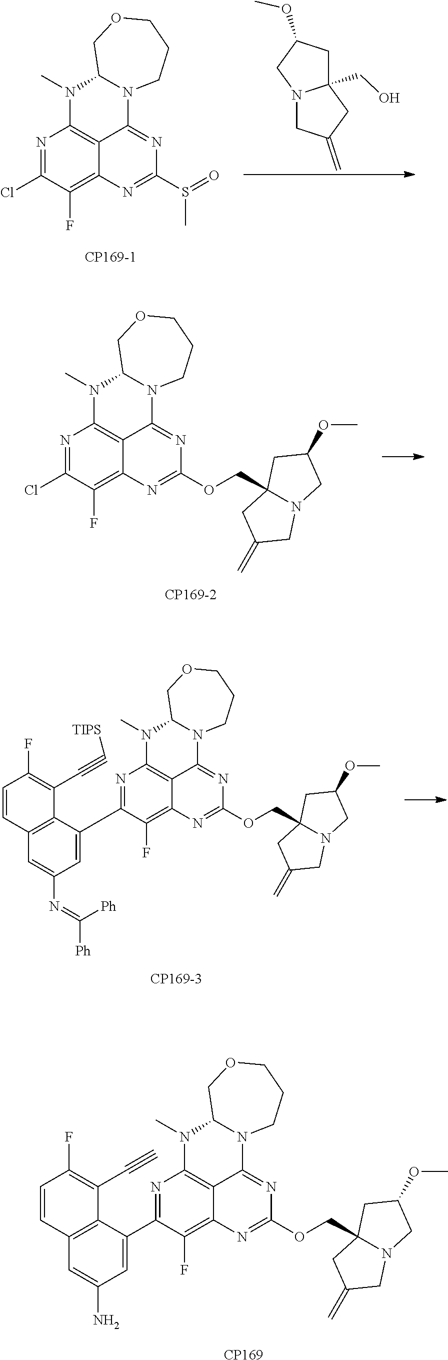

Compound 169 (CP169, confirmed, MS: m/z: 640[M+H]⁺) was synthesized in a manner similar to CP46.

### Example 170

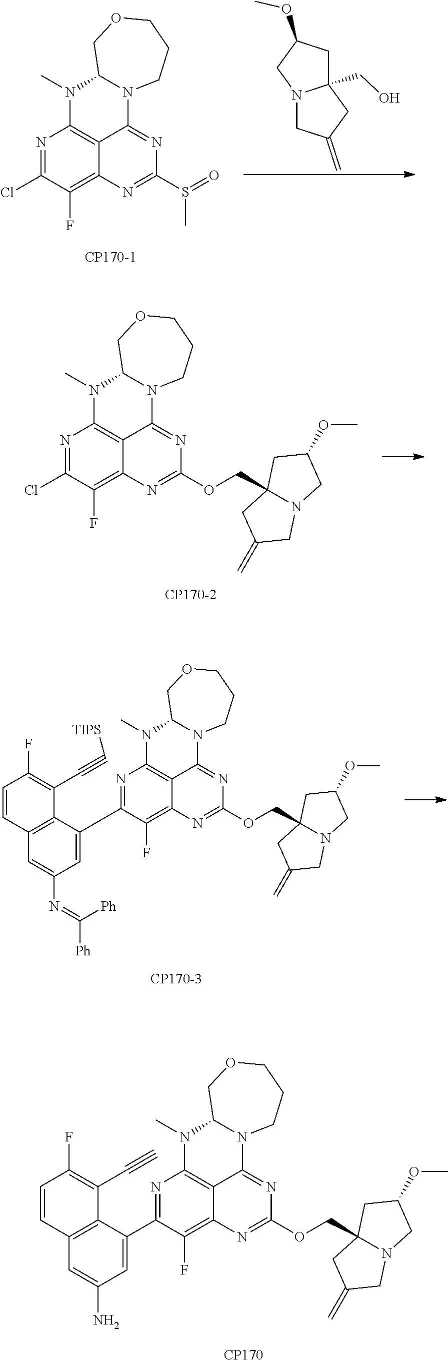

Compound 170 (CP170, confirmed, MS: m/z: 640[M+H]⁺) was synthesized in a manner similar to CP46.

### Example 171

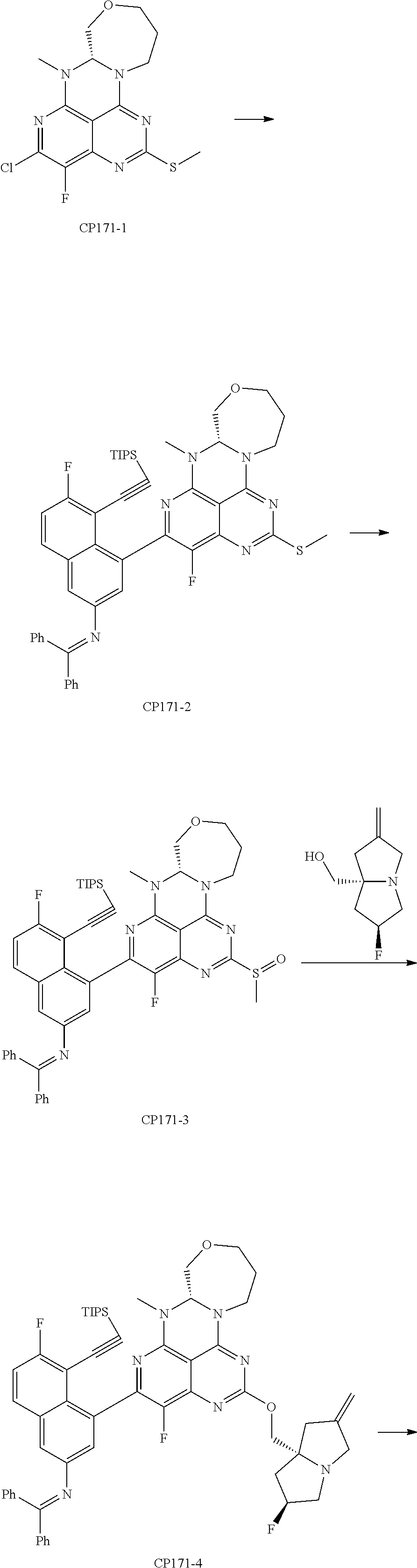

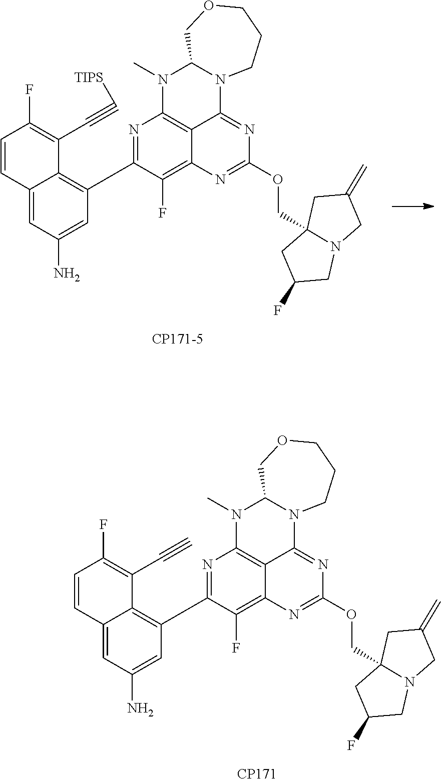

Compound 171 (CP171, confirmed, MS: m/z: 628[M+H]⁺) was synthesized in a manner similar to CP46.

### Example 172

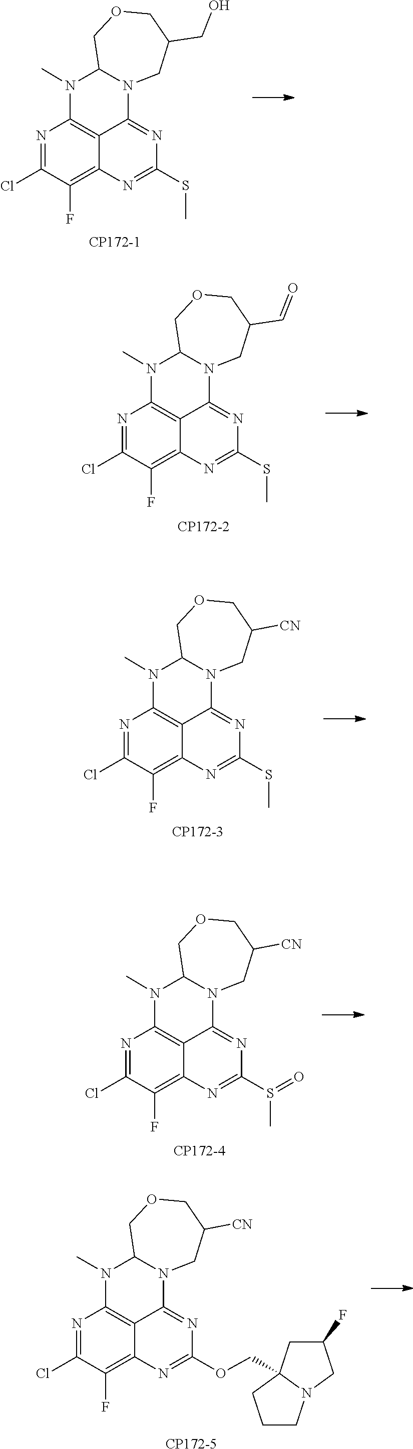

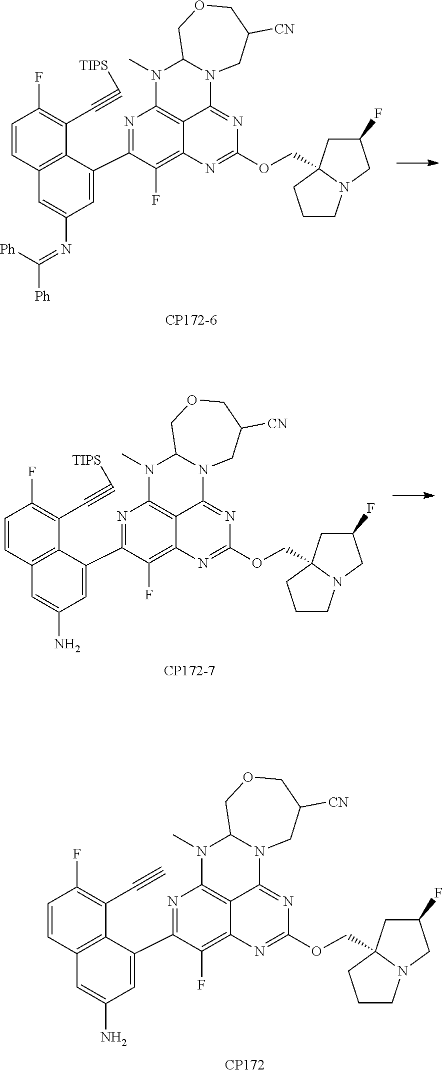

Compound 172 (CP172, confirmed. MS m/z: 641 [M+H]⁺) was synthesized in a manner similar to CP46.

### Example 173

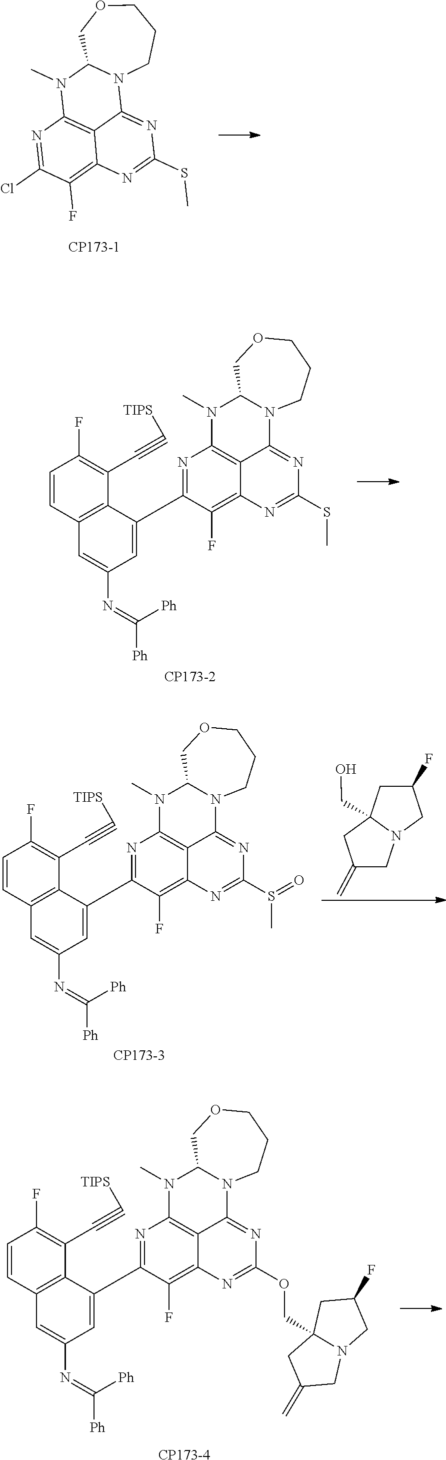

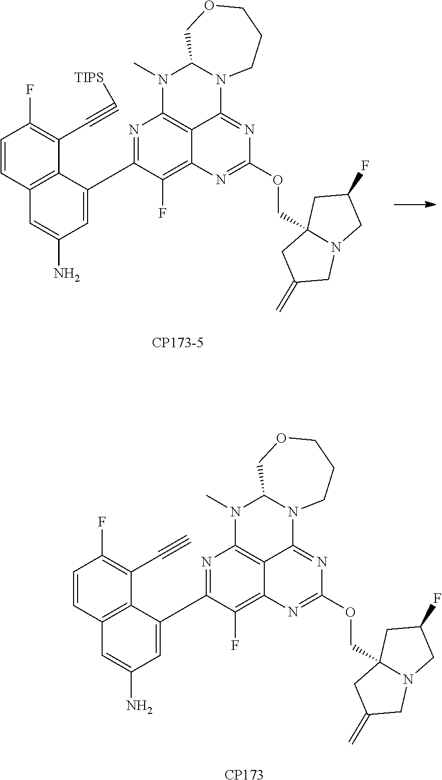

Compound 173 (CP173, confirmed, MS: m/z: 628 [M+H]⁺) was synthesized in a manner similar to CP46.

### Example 174

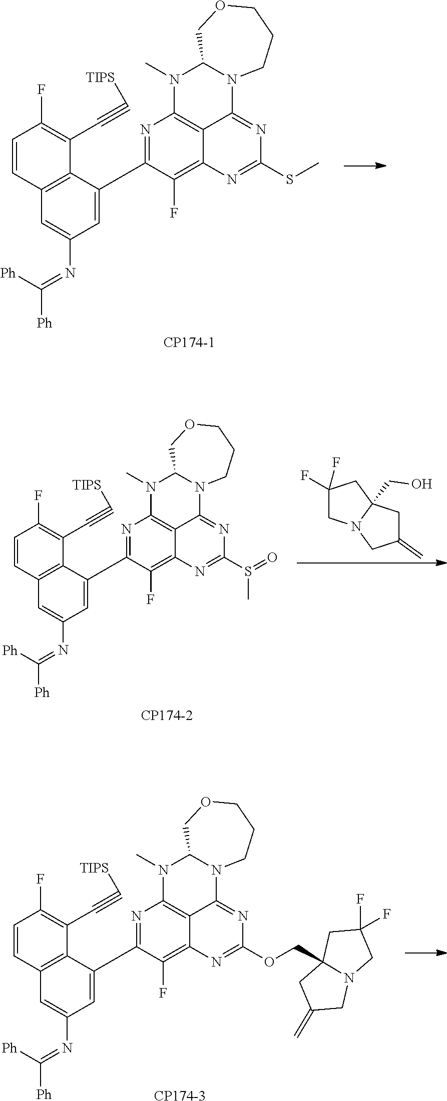

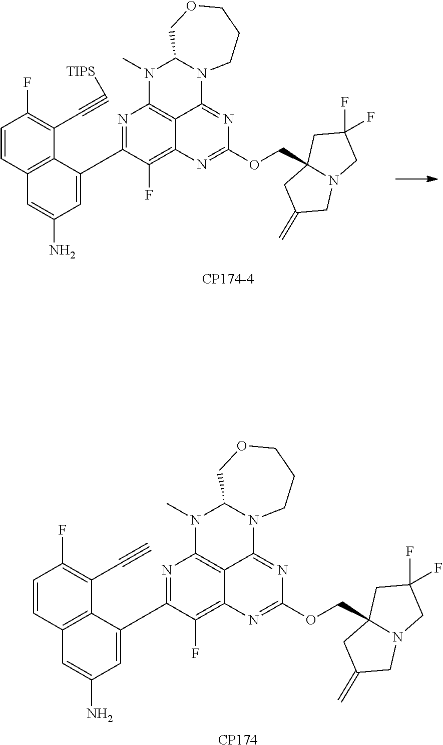

Compound 174 (CP173, confirmed, MS: m/z: 628[M+H]⁺) was synthesized in a manner similar to CP46.

### Example 175

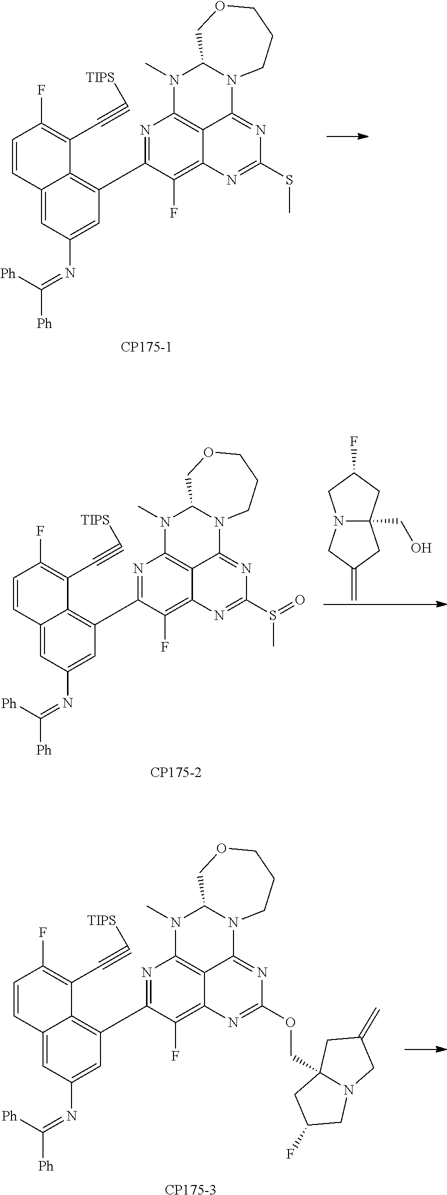

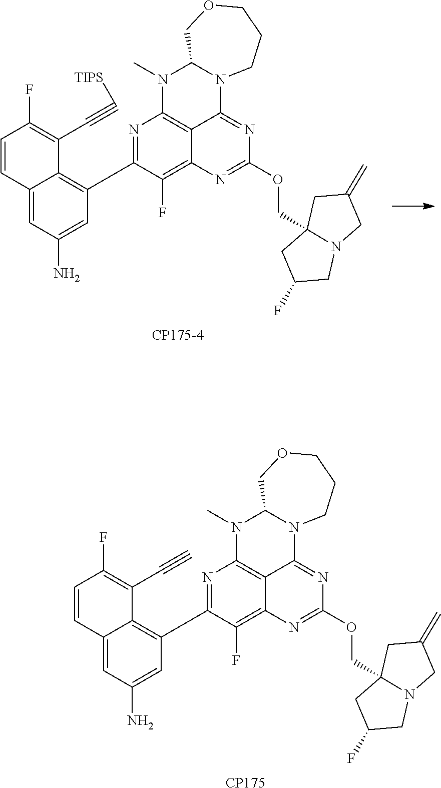

Compound 175 (CP175, confirmed, MS: m/z: 628[M+H]⁺) was synthesized in a manner similar to CP46.

### Example 176

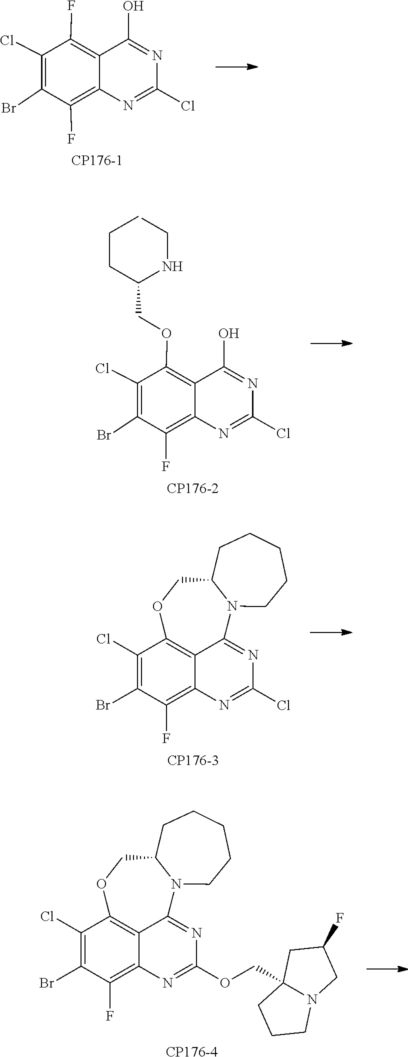

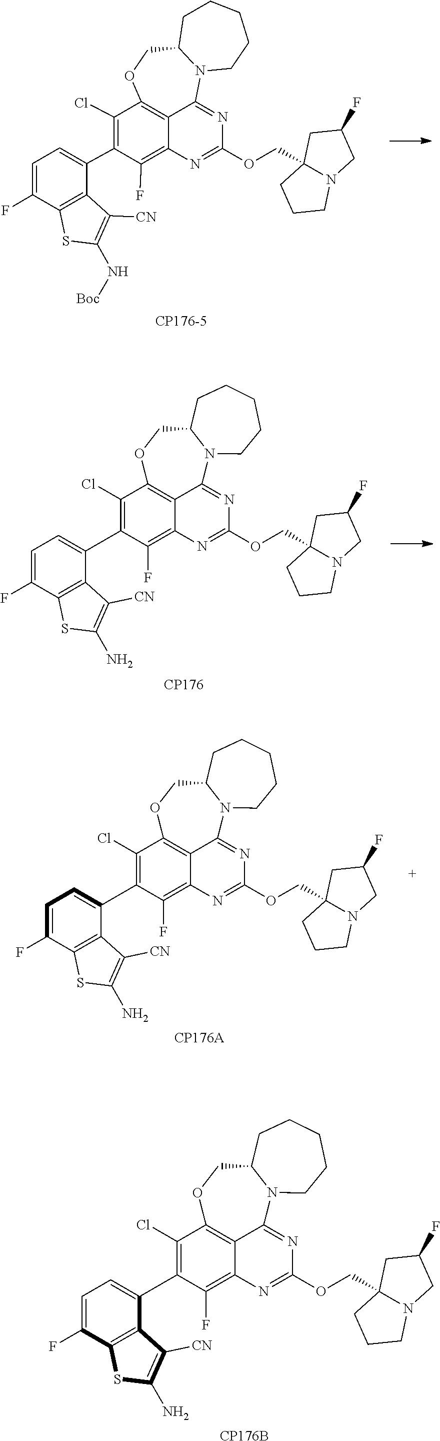

Compound 176 (CP176) was synthesized in a manner similar to CP66 and separated by Prep-HPLC (Agela Durashell C18, 30 mm×250 mm, 10 um, A: 0.05% NH₃·H₂O in water, B: CH₃CN, Gradient: 30% B to 74% B in 39 mins at a flow rate of 40 mL/min, 240 nm) to afford Compound 176A (CP176A, confirmed) (1.2 mg. TFA salt) and Compound 176B (CP176B, Compound 139, confirmed) (1.0 mg). MS m/z: 655 [M+H]⁺.

### Example 177

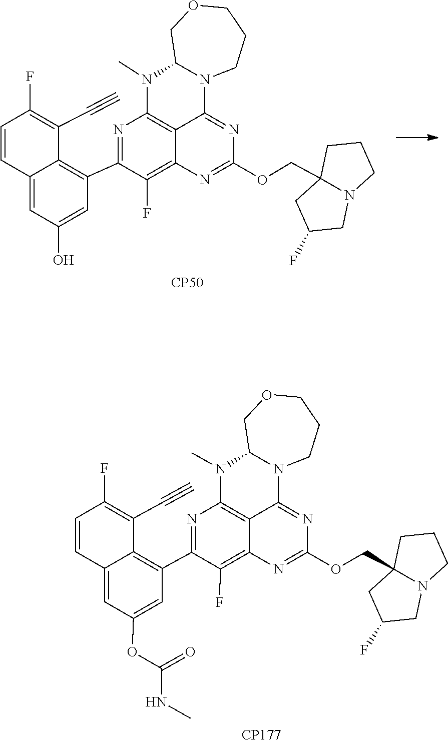

Compound 177 (CP177) was synthesized with CP50 and anhydride or acyl chloride. Compound 177 is the prodrug of CP50. It was observed that Compound 177 was converted to the active ingredient of CP50 in vivo assay.

¹HNMR of exemplary compounds are listed in the following table:

| Compound | ¹HNMR |
|---|---|
| Compound 10B | ¹HNMR(400 MHz, MeOD)δ7.68(ddd, J = 8.3, 5.9, 2.2 Hz, 1H), 7.24(td, J = 9.0, 3.9 Hz, 1 |
| | H), 7.01(dt, J = 9.1, 2.3 Hz, 2H), 5.31(s, 1H), 5.17(dt, J = 9.6, 4.6 Hz, 2H), 4.49(ddd, J = |
| | 17.1, 9.5, 4.1 Hz, 1H), 4.05-3.91(m, 4H), 3.52(ddd, J = 24.8, 11.9, 10.1 Hz, 1H), 3.45-3.33(m, |
| | 1H), 3.33-3.22(m, 1H), 3.17-3.13(m, 2H), 3.10(d, J = 12.8 Hz, 2H), 3.06-3.01(m, 1H), |
| | 3.01(s, 3H), 2.89-2.80(m, 1H), 2.27(tt, J = 20.5, 8.0 Hz, 1H), 2.18-2.10(m, 1H), 2.07(dd, |
| | J = 7.7, 3.2 Hz, 1H), 2.03-1.91(m, 2H), 1.81(ddd, J = 24.2, 16.2, 7.5 Hz, 3H). |
| Compound 14A | ¹HNMR(400 MHz, DMSO-d₆)δ7.82-7.61(m, 1H), 7.39-7.24(m, 1H), 7.10-6.97(m, 2 |
| | H), 5.62(s, 2H), 5.41-5.16(m, 1H), 4.95-4.80(m, 1H), 4.61-4.44(m, 1H), 4.30-4.19(m, 2 |
| | H), 4.15-3.95(m, 2H), 3.83(d, 2H), 3.22-2.98(m, 3H), 2.84(d, 1H), 2.40-1.56(m, 10H), |
| | 1.47-1.11(m, 4H). |
| Compound 14B | ¹HNMR(400 MHz, CDCl₃)δ7.56(dt, J = 35.3, 17.7 Hz, 1H), 7.12(td, J = 8.8, 5.7 Hz, 1H), |
| | 7.02(t, J = 4.0 Hz, 2H), 6.08(d, J = 5.2 Hz, 1H), 5.36-5.13(m, 2H), 4.69(dd, J = 12.7, 3.6 Hz, |
| | 1H), 4.29(dd, J = 10.7, 4.4 Hz, 2H), 3.84(s, 1H), 3.38(s, 1H), 3.25-3.08(m, 2H), 2.97(s, 1 |
| | H), 2.76(dd, J = 15.0, 12.2 Hz, 1H), 2.36-2.13(m, 4H), 2.01-1.80(m, 5H), 1.80-1.60(m, 5 |
| | H), 1.55(d, J = 10.8 Hz, 1H), 1.39-1.27(m, 1H), 1.18(s, 1H). |
| Compound 30 | ¹HNMR(400 MHz, DMSO-d₆)δ7.84-7.69(m, 1H), 7.42-7.26(m, 1H), 7.16-6.90(m, 2 |
| | H), 5.57(d, 2H), 5.06(d, 1H), 4.92(d, 1H), 4.71-4.36(m, 6H), 3.97-3.61(m, 5H), 3.15- |
| | 2.97(m, 1H), 2.39-1.92(m, 6H), 1.86-1.58(m, 4H), 1.23-1.09(m, 3H). |
| Compound 36 | ¹HNMR(400 MHz, DMSO-d₆)δ7.82-7.74(m, 1H), 7.38-7.30(m, 1H), 7.10-6.96(m, 2 |
| | H), 5.57(d, 2H), 5.33-5.15(m, 1H), 4.63-4.42(m, 2H), 4.18-3.97(m, 3H), 3.96-3.66(m, |
| | 5H), 3.60-3.43(m, 1H), 3.39-3.15(m, 2H), 3.00(d, 3H), 2.64-2.39(m, 2H), 2.38-1.95(m, |
| | 6H), 1.82-1.67(m, 3H). |
| Compound 38A | ¹HNMR(400 MHz, DMSO-d₆)δ7.75(dd, J = 9.2, 5.9 Hz, 1H), 7.33(t, J = 8.7 Hz, 1H), 7.07- |
| | 6.98(m, 2H), 5.62(d, J = 6.9 Hz, 2H), 5.29(d, J = 54.1 Hz, 1H), 4.68-4.52(m, 1H), 4.50- |
| | 4.36(m, 2H), 4.26(dd, J = 15.1, 10.6 Hz, 1H), 4.21-4.10(m, 2H), 4.04-3.92(m, 3H), 3.67- |
| | 3.48(m, 1H), 3.43-3.34(m, 1H), 3.10(d, J = 7.1 Hz, 2H), 3.04(d, J = 11.4 Hz, 1H), 2.84(t, |
| | J = 7.2 Hz, 1H), 2.38(dt, J = 21.1, 10.4 Hz, 1H), 2.18-2.10(m, 1H), 2.09-1.95(m, 3H), 1.82(dd, |
| | J = 23.0, 10.8 Hz, 3H), 1.73(dd, J = 6.5, 4.9 Hz, 3H). |
| Compound 39B | ¹HNMR(400 MHz, DMSO-d₆)δ7.78-7.63(m, 1H), 7.36-7.27(m, 1H), 7.07-6.95(m, 2 |
| | H), 5.59(s, 2H), 5.40-4.72(m, 1H), 4.28-4.17(m, 2H), 4.17-3.89(m, 4H), 3.82(s, 2H), |
| | 3.79-3.52(m, 3H), 3.18-2.75(m, 7H), 2.18-1.16(m, 6H). |
| Compound 40A | ¹HNMR(400 MHz, DMSO-d₆)δ7.81-7.67(m, 1H), 7.37-7.23(m, 1H), 7.11-6.93(m, 2 |
| | H), 5.65-5.51(m, 2H), 5.39-5.18(m, 2H), 4.98-4.76(m, 1H), 4.49-4.36(m, 1H), 4.19- |
| | 3.88(m, 3H), 3.31-3.21(m, 1H), 3.16-3.05(m, 5H), 3.03(d, J = 10.5 Hz, 1H), 2.89-2.77(m, |
| | 1H), 2.16-1.89(m, 5H), 1.88-1.69(m, 6H), 1.49-1.29(m, 1H). |
| Compound 41 | ¹HNMR(400 MHz, DMSO-d₆)δ10.78(s, 1H), 7.77(dd, J = 9.0, 5.9 Hz, 1H), 7.37-7.28(m, |
| | 1H), 7.09-6.92(m, 2H), 5.56(d, J = 51.2 Hz, 1H), 5.47-5.39(m, 1H), 5.06-5.00(m, 1H), |
| | 4.60-4.48(m, 3H), 4.10(s, 1H), 3.98-3.66(m, 7H), 3.38-3.12(m, 2H), 3.08-2.95(m, 3H), |
| | 2.43-2.25(m, 3H), 2.24-2.10(m, 2H), 2.10-1.95(m, 1H), 1.95-1.79(m, 1H), 1.39-1.27(m, |
| | 3H). |
| Compound 42 | ¹HNMR(400 MHz, DMSO-d₆)δ10.99-10.53(m, 1H), 7.82-7.74(m, 1H), 7.38-7.29(m, |
| | 1H), 7.11-6.94(m, 2H), 5.63(s, 1H), 5.51(s, 1H), 5.04-4.92(m, 1H), 4.65-4.46(m, 2H), |
| | 4.46-4.33(m, 1H), 4.09-3.97(m, 1H), 3.93-3.60(m, 5H), 3.43-3.21(m, 3H), 3.17-3.00(m, |
| | 3H), 2.62-2.53(m, 1H), 2.39-2.23(m, 2H), 2.23-2.09(m, 2H), 2.09-1.86(m, 3H), 1.22- |
| | 1.13(m, 3H). |
| Compound 43 | ¹HNMR(400 MHz, DMSO-d₆)δ10.88(m, 1H), 7.83-7.76(m, 1H), 7.39-7.30(m, 1H), 7.13- |
| | 6.99(m, 2H), 5.67-5.43(m, 1H), 5.02-4.91(m, 1H), 4.62-4.50(m, 2H), 4.46-4.31(m, |
| | 1H), 4.08-3.97(m, 1H), 3.91-3.64(m, 5H), 3.42-3.23(m, 3H), 3.14-3.07(m, 3H), 2.47(s, |
| | 1H), 2.38-1.85(m, 7H), 1.22-1.11(m, 3H). |
| Compound 45 | ¹HNMR(400 MHz, DMSO-d₆)δ7.82-7.74(m, 1H), 6.98(t, J = 13.4 Hz, 2H), 5.57(d, J = |
| | 53.4 Hz, 2H), 5.37-5.29(m, 1H), 4.57(td, J = 22.7, 12.0 Hz, 2H), 4.50-4.41(m, 1H), 4.08- |
| | 3.93(m, 2H), 3.93-3.81(m, 2H), 3.76(d, J = 4.4 Hz, 2H), 3.55(ddd, J = 17.0, 12.1, 6.5 Hz, 1H), |
| | 3.37(dd, J = 31.8, 11.4 Hz, 3H), 3.01(d, J = 4.4 Hz, 3H), 2.58(d, J = 15.7 Hz, 1H), 2.47(s, 1 |
| | H), 2.37-2.23(m, 2H), 2.25(s, 3H), 2.04(d, J = 27.1 Hz, 2H). |
| Compound 46 | ¹HNMR(400 MHz, DMSO-d₆)δ7.81-7.64(m, 1H), 7.31(d, 1H), 7.05-6.91(m, 2H), 5.58(s, |
| | 2H), 5.43-5.15(m, 1H), 4.55-4.23(m, 1H), 4.22-3.86(m, 4H), 3.85-3.37(m, 3H), 3.24- |
| | 3.00(m, 4H), 3.00-2.94(m, 3H), 2.91-2.73(m, 2H), 2.39-1.90(m, 4H), 1.92-1.64(m, 3 |
| | H), 1.17-0.92(m, 3H). |
| Compound 51 | ¹HNMR(400 MHz, DMSO-d₆)δ7.68-7.63(m, 1H), 7.30-7.24(m, 2H), 6.96(d, J = 1.6 Hz, |
| | 2H), 5.60(s, 2H), 5.37-5.19(m, 2H), 4.51-4.40(m, 1H), 4.13-4.06(m, 1H), 3.99(s, 3H), |
| | 3.84-3.80(m, 1H), 3.60-3.35(m, 2H), 3.30-3.21(m, 1H), 3.13-2.94(m, 6H), 2.87-2.78(m, |
| | 1H), 2.30-2.03(m, 3H), 1.99-1.71(m, 5H). |
| Compound 52B | ¹HNMR(400 MHz, DMSO-d₆)δ7.80-7.60(m, 1H), 7.44-7.14(m, 1H), 7.09-7.00(m, 1 |
| | H), 6.99-6.82(m, 1H), 5.61(s, 1H), 5.42-5.29(m, 1H), 5.24(s, 1H), 4.22(t, J = 5.8 Hz, 1H), |
| | 4.18-3.93(m, 3H), 3.83(s, 1H), 3.55(t, J = 11.2 Hz, 1H), 3.41(dd, J = 23.7, 16.1 Hz, 1H), |
| | 3.12(s, 1H), 3.00(d, J = 3.3 Hz, 3H), 2.85(s, 1H), 2.75-2.61(m, 1H), 2.20(dd, J = 23.9, 16.6 |
| | Hz, 1H), 2.15-1.92(m, 2H), 1.79(s, 2H), 1.70-1.54(m, 1H), 1.44-1.31(m, 3H), 1.30(s, 1H), |
| | 1.25(d, J = 3.7 Hz, 1H), 1.23-1.20(m, 1H). |
| Compound 53 | ¹HNMR(400 MHz, DMSO-d₆)δ10.75(s, 1H), 7.82-7.70(m, 1H), 7.33(td, J = 9.0, 5.1 Hz, |
| | 1H), 7.09-6.93(m, 2H), 5.64(s, 1H), 5.50(s, 1H), 5.42-5.32(m, 1H), 5.40-5.30(m, 1H), |
| | 5.19-4.99(m, 2H), 4.66-4.48(m, 3H), 3.89-3.55(m, 5H), 3.35-3.21(m, 1H), 3.01(s, 3H), |
| | 2.39-2.26(m, 3H), 2.25-2.10(m, 3H), 2.07-1.85(m, 2H). |
| Compound 54B | ¹HNMR(400 MHz, DMSO-d₆)δ7.84-7.71(m, 1H), 7.40-7.29(m, 1H), 7.09(s, 1H), 7.01(s, |
| | 1H), 5.63(s, 1H), 5.50(s, 1H), 5.22-5.14(m, 1H), 4.67-4.32(m, 4H), 3.95-3.68(m, 3 |
| | H), 3.40-3.17(m, 2H), 3.06-2.92(m, 3H), 2.47(s, 1H), 2.38-2.27(m, 1H), 2.25-2.10(m, 3 |
| | H), 2.08-1.93(m, 2H), 1.91-1.56(m, 5H), 1.57-1.39(m, 1H), 1.36-1.13(m, 2H). |
| Compound 55 | ¹HNMR(400 MHz, DMSO-d₆-d₆)δ7.83-7.68(m, 1H), 7.39-7.25(m, 1H), 7.08-6.94(m, |
| | 2H), 5.67-5.53(m, 2H), 5.49-5.38(m, 1H), 5.07-4.90(m, 1H), 4.27-4.04(m, 4H), 3.99- |
| | 3.85(m, 2H), 3.85-3.69(m, 2H), 3.29-3.05(m, 2H), 3.01(d, J = 7.9 Hz, 3H), 2.36-1.69(m, |
| | 6H), 1.69-1.32(m, 1H), 1.30-1.17(m, 1H), 0.99-0.78(m, 1H). |
| Compound 56A | ¹HNMR(400 MHz, DMSO-d₆)δ7.77-7.69(m, 1H), 7.40-7.23(m, 1H), 7.06-6.92(m, 2 |
| | H), 5.58(s, 2H), 5.37-5.17(m, 2H), 4.44-4.30(m, 1H), 4.13-3.98(m, 2H), 3.88-3.75(m, 2 |
| | H), 3.71-3.59(m, 1H), 3.60-3.43(m, 1H), 3.13-3.06(m, 2H), 3.05(s, 1H), 3.02(s, 1H), |
| | 2.99-2.95(m, 3H), 2.89-2.76(m, 1H), 2.39-2.22(m, 1H), 2.18-2.09(m, 1H), 2.07-1.97(m, |
| | 2H), 1.90-1.72(m, 5H), 1.71-1.55(m, 2H). |
| Compound 58 | ¹HNMR(400 MHz, DMSO-d₆)δ7.74(dd, J = 9.2, 6.0 Hz, 1H), 7.31(t, J = 9.0 Hz, 1H), 6.98 |
| | (dd, J = 24.1, 3.5 Hz, 2H), 5.59(s, 2H), 5.46-5.12(m, 3H), 4.27-4.11(m, 2H), 4.10(d, J = |
| | 10.3 Hz, 1H), 3.93(ddd, J = 30.7, 15.9, 7.9 Hz, 2H), 3.58(t, J = 11.3 Hz, 1H), 3.18-3.00(m, |
| | 3H), 2.98(d, J = 4.6 Hz, 3H), 2.84(d, J = 5.6 Hz, 1H), 2.12(dd, J = 14.7, 9.4 Hz, 1H), 2.04(d, |
| | J = 11.2 Hz, 1H), 2.00(s, 1H), 1.90(t, J = 14.5 Hz, 2H), 1.80(dd, J = 9.7, 6.9 Hz, 5H), |
| | 1.69(s, 1H). |
| Compound 59A | ¹HNMR(400 MHz, DMSO-d₆)δ10.82(s, 1H), 7.77(dd, J = 9.0, 6.0 Hz, 1H), 7.34(t, J = 9.0 |
| | Hz, 1H), 7.05(s, 1H), 6.98(s, 1H), 5.64(s, 1H), 5.51(s, 1H), 5.33-5.18(m, 2H), 4.63- |
| | 4.51(m, 2H), 4.51-4.39(m, 2H), 4.32(dd, J = 12.4, 9.3 Hz, 1H), 4.15(s, 1H), 4.13-4.00(m, 2H), |
| | 3.91(dd, J = 16.3, 10.3 Hz, 2H), 3.78(m, 3H), 3.58-3.42(m, 1H), 3.32(d, J = 9.7 Hz, 3H), |
| | 3.30(s, 1H), 2.99(dd, J = 17.7, 4.3 Hz, 3H), 2.47(s, 1H), 2.41-2.28(m, 2H), 2.24-1.93(m, 4H). |
| Compound 60 | ¹HNMR(400 MHz, DMSO-d₆)δ7.78-7.69(m, 1H), 7.31(t, J = 9.0 Hz, 1H), 7.06-6.93(m, |
| | 2H), 5.60(s, 2H), 5.27(d, J = 54.5 Hz, 1H), 5.17-5.03(m, 1H), 4.14-3.96(m, 4H), 3.56- |
| | 3.45(m, 1H), 3.43-3.33(m, 1H), 3.07(s, 2H), 3.02-2.94(m, 4H), 2.91-2.73(m, 2H), 2.72- |
| | 2.56(m, 1H), 2.26-1.92(m, 5H), 1.90-1.66(m, 3H), 1.03-0.95(m, 1H), 0.69-0.56(m, 1H), |
| | 0.56-0.39(m, 2H), 0.35-0.20(m, 1H). |
| Compound 64 | ¹HNMR(400 MHz, DMSO-d₆)δ7.76(dd, J = 9.1, 5.9 Hz, 1H), 7.33(td, J = 9.1, 2.8 Hz, 1H), |
| | 7.08-6.96(m, 2H), 5.70-5.53(m, 2H), 5.27(d, J = 54.7 Hz, 1H), 4.67-4.52(m, 1H), 4.49- |
| | 4.34(m, 1H), 4.28-3.87(m, 6H), 3.68-3.40(m, 1H), 3.14-2.94(m, 3H), 2.83(s, 1H), 2.68- |
| | 2.54(m, 1H), 2.30-1.89(m, 5H), 1.90-1.68(m, 3H), 1.38-1.12(m, 2H), 0.69-0.53(m, 1H), |
| | 0.55-0.37(m, 2H), 0.39-0.24(m, 1H). |
| Compound 74 | ¹HNMR(400 MHz, DMSO-d₆)δ7.80-7.65(m, 1H), 7.31(td, J = 9.0, 3.8 Hz, 1H), 7.10-6.90(m, |
| | 2H), 5.58(s, 2H), 5.25(ddd, J = 10.3, 6.8, 3.7 Hz, 1H), 4.52-4.38(m, 1H), 4.31-4.14 |
| | (m, 2H), 4.07-3.84(m, 2H), 3.62-3.43(m, 5H), 3.41-3.32(m, 2H), 3.30-3.22(m, 1H), |
| | 2.99(d, J = 5.1 Hz, 3H), 2.36(d, J = 23.7 Hz, 4H), 2.33-2.16(m, 3H), 1.96(s, 1H), 0.63(s, |
| | 2H), 0.41(s, 2H). |
| | ¹⁹FNMR(377 MHz, DMSO-d₆)δ −113.36(d, J = 18.2 Hz), −153.13-−157.77(m). |
| Compound 75 | ¹HNMR(400 MHz, DMSO-d₆)δ7.99(s, 2H), 7.32(dd, J = 7.9, 5.5 Hz, 1H), 7.04(t, J = 8.9 |
| | Hz, 1H), 5.28(dd, J = 30.5, 20.0 Hz, 2H), 4.44(s, 1H), 4.13(d, J = 10.4 Hz, 1H), 4.07- |
| | 3.84(m, 3H), 3.51(dd, J = 22.3, 12.0 Hz, 1H), 3.12-2.97(m, 6H), 2.83(dd, J = 14.9, 8.6 Hz, 1H), |
| | 2.19(d, J = 4.4 Hz, 1H), 2.15(d, J = 4.3 Hz, 1H), 2.12(s, 1H), 2.06(d, J = 4.4 Hz, 1H), 2.03- |
| | 1.88(m, 3H), 1.87-1.68(m, 3H). |
| Compound 80 | ¹HNMR(400 MHz, DMSO-d₆)δ7.78-7.72(m, 1H), 7.36-7.28(m, 1H), 7.08-6.97(m, 2 |
| | H), 6.24(s, 1H), 5.69-5.54(m, 3H), 5.27(d, J = 53.0 Hz, 1H), 4.72(s, 1H), 4.16-3.92(m, 2 |
| | H), 3.17-3.05(m, 5H), 3.01(s, 1H), 2.83(s, 1H), 2.35-2.09(m, 3H), 2.09-1.93(m, 4H), |
| | 1.88-1.71(m, 3H), 1.53-1.48(m, 2H). |
| Compound 83 | ¹HNMR(400 MHz, DMSO-d₆)δ7.79(ddd, J = 9.4, 5.9, 3.8 Hz, 1H), 7.34(td, J = 9.0, 5.2 Hz, |
| | 1H), 7.09(s, 1H), 7.02(d, J = 2.3 Hz, 1H), 5.63(s, 1H), 5.51(s, 1H), 5.32-5.20(m, 1H), |
| | 5.16-4.90(m, 1H), 4.65-4.52(m, 2H), 4.41(t, J = 15.3 Hz, 1H), 3.88(s, 1H), 3.85-3.68(m, 2 |
| | H), 3.38-3.19(m, 2H), 3.03-2.86(m, 3H), 2.69-2.51(m, 1H), 2.47(s, 1H), 2.34(dd, J = |
| | 21.1, 15.7 Hz, 2H), 2.26-2.10(m, 3H), 2.02(d, J = 6.0 Hz, 2H), 2.00-1.83(m, 3H), 1.52(d, |
| | J = 7.0 Hz, 1H), 1.23(s, 1H). |
| Compound 85 | ¹HNMR(400 MHz, DMSO-d₆)δ7.73(dt, J = 9.6, 4.9 Hz, 1H), 7.36-7.25(m, 1H), 7.15-6.94(m, |
| | 2H), 6.02(d, J = 16.1 Hz, 1H), 5.59(s, 2H), 5.28(d, J = 53.9 Hz, 1H), 4.61(dd, J = 33.7, |
| | 14.2 Hz, 1H), 4.16-4.07(m, 1H), 4.05(t, J = 5.4 Hz, 1H), 4.01-3.93(m, 1H), 3.09(q, J = |
| | 14.1 Hz, 2H), 3.02(s, 1H), 2.98-2.87(m, 1H), 2.83(d, J = 5.4 Hz, 3H), 2.36(t, J = 14.0 Hz, |
| | 1H), 2.12(s, 2H), 2.03(d, J = 17.0 Hz, 2H), 1.96-1.81(m, 3H), 1.80-1.70(m, 2H), 1.63(dd, J = |
| | 14.7, 6.8 Hz, 1H), 1.53-1.39(m, 1H), 1.40-1.18(m, 2H). |
| Compound 86 | ¹HNMR(400 MHz, DMSO-d₆)δ7.73(dd, J = 9.1, 6.0 Hz, 1H), 7.29(dd, J = 17.4, 8.4 Hz, 1 |
| | H), 7.18-6.95(m, 2H), 6.01(d, J = 16.2 Hz, 1H), 5.72-5.45(m, 2H), 5.27(d, J = 54.7 Hz, |
| | 1H), 4.60(dd, J = 34.1, 15.0 Hz, 1H), 4.15-4.07(m, 1H), 4.03(d, J = 7.5 Hz, 1H), 3.94(dd, J = |
| | 22.1, 8.5 Hz, 1H), 3.15-3.05(m, 2H), 3.02(d, J = 10.5 Hz, 1H), 2.90(d, J = 13.0 Hz, 1H), |
| | 2.83(d, J = 5.5 Hz, 3H), 2.35(t, J = 13.9 Hz, 1H), 2.12(s, 2H), 2.02(d, J = 16.5 Hz, 2H), 1.88(d, |
| | J = 26.2 Hz, 2H), 1.78(dd, J = 12.3, 7.8 Hz, 2H), 1.69-1.53(m, 1H), 1.45(s, 1H), 1.19(d, J = 29.8 |
| | Hz, 1H), 1.03-0.88(m, 2H). |
| Compound 88 | ¹HNMR(400 MHz, DMSO-d₆)δ7.85-7.61(m, 1H), 7.31(t, J = 8.5 Hz, 1H), 6.99(t, J = 14.7 |
| | Hz, 2H), 5.58(s, 2H), 5.29(d, J = 54.7 Hz, 1H), 5.03(s, 1H), 4.06(t, J = 10.7 Hz, 2H), 3.94(d, |
| | J = 55.9 Hz, 1H), 3.73(s, 1H), 3.08(d, J = 29.8 Hz, 3H), 2.91(t, J = 8.4 Hz, 3H), 2.85(s, 1H), |
| | 2.10(d, J = 26.7 Hz, 2H), 1.96(d, J = 25.6 Hz, 3H), 1.79(d, J = 6.1 Hz, 3H), 1.67-1.60(m, 3 |
| | H), 1.46(d, J = 11.8 Hz, 2H), 1.24(s, 2H), 1.15-0.96(m, 1H), 0.88(s, 1H). |
| Compound 91A | ¹HNMR(400 MHz, DMSO-d₆)δ7.79-7.70(m, 1H), 7.36-7.27(m, 1H), 7.07-6.98(m, 2 |
| | H), 6.09-5.92(m, 1H), 5.87(s, 1H), 5.79-5.70(m, 1H), 5.59(s, 2H), 5.29(d, J = 53.7 Hz, 1 |
| | H), 4.81-4.63(m, 1H), 4.16-4.05(m, 1H), 4.03-3.94(m, 1H), 3.92-3.68(m, 1H), 3.18- |
| | 2.95(m, 6H), 2.90-2.76(m, 1H), 2.43-2.28(m, 1H), 2.26-1.69(m, 10H). |
| Compound 91B | ¹HNMR(400 MHz, DMSO-d₆)δ7.79-7.71(m, 1H), 7.36-7.26(m, 1H), 7.06-6.97(m, 2 |
| | H), 6.08-5.92(m, 1H), 5.87(s, 1H), 5.80-5.71(m, 1H), 5.59(s, 2H), 5.28(d, J = 53.7 Hz, 1 |
| | H), 4.79-4.64(m, 1H), 4.11-3.96(m, 2H), 3.93-3.70(m, 1H), 3.38-3.34(m, 1H), 3.18- |
| | 3.06(m, 2H), 3.05-2.95(m, 4H), 2.90-2.78(m, 1H), 2.46-2.30(m, 1H), 2.26-1.98(m, 4H), |
| | 1.95-1.73(m, 5H). |
| Compound 92A | ¹HNMR(400 MHz, DMSO-d₆)δ7.73(s, 1H), 7.31(s, 1H), 6.99(d, J = 13.2 Hz, 2H), 5.58(s, |
| | 2H), 5.42-5.12(m, 2H), 4.90-4.49(m, 1H), 4.16(d, J = 44.0 Hz, 2H), 4.06(s, 3H), 3.81(dd, |
| | J = 91.3, 31.1 Hz, 2H), 3.56-3.37(m, 3H), 3.07(d, J = 24.6 Hz, 3H), 2.97(s, 3H), 2.83(s, 1 |
| | H), 2.12(s, 1H), 2.04(s, 2H), 1.82(d, J = 24.9 Hz, 2H), 1.74(d, J = 37.0 Hz, 2H), 1.23(s, 1H). |
| Compound 94 | 1HNMR(400 MHz, DMSO-d₆): δ7.73(dt, J = 18.1, 9.1 Hz, 1H), 7.32(t, J = 9.0 Hz, 1H), |
| | 6.99(dd, J = 22.7, 6.5 Hz, 2H), 5.59(s, 2H), 5.38-5.09(m, 2H), 4.16(d, J = 27.8 Hz, 1H), 4.14- |
| | 3.90(m, 4H), 3.69-3.44(m, 3H), 3.22(s, 2H), 3.17-2.78(m, 7H), 2.45-2.32(m, 2H), 2.23- |
| | 1.69(m, 9H). |
| Compound 95 | 1HNMR(400 MHz, DMSO-d₆)δ7.75(dd, J = 8.9, 6.2 Hz, 1H), 7.31 (td, J = 9.0, 4.4 Hz, 1H), |
| | 7.03(dd, J = 22.2, 5.8 Hz, 2H), 5.59(s, 2H), 5.30(dd, J = 30.4, 23.2 Hz, 2H), 4.42(d, J = 4.5 |
| | Hz, 1H), 4.21-3.87(m, 3H), 3.13-3.00(m, 5H), 2.90-2.76(m, 1H), 2.06(dd, J = 31.8, 21.3 |
| | Hz, 4H), 1.82(ddd, J = 33.4, 29.6, 17.3 Hz, 9H), 1.69(s, 1H). |
| Compound 99 | ¹HNMR(400 MHz, DMSO-d₆)δ7.78-7.63(m, 2H), 7.31(dd, J = 10.3, 7.8 Hz, 1H), 6.99(d, |
| | J = 11.5 Hz, 1H), 6.13-5.83(m, 1H), 5.77-5.50(m, 2H), 5.28(dd, J = 32.0, 20.9 Hz, 1H), |
| | 4.76(dt, J = 13.6, 6.7 Hz, 1H), 4.45(d, J = 13.4 Hz, 1H), 4.21(dd, J = 17.7, 11.1 Hz, 1H), 4.15- |
| | 3.86(m, 2H), 3.74(d, J = 15.8 Hz, 1H), 3.10(dd, J = 31.2, 22.5 Hz, 1H), 2.96(dd, J = 6.0, 3.0 |
| | Hz, 1H), 2.84(d, J = 6.9 Hz, 1H), 2.70-2.58(m, 3H), 2.31(d, J = 15.2 Hz, 1H), 2.23-1.94(m, |
| | 2H), 1.90-1.69(m, 3H), 1.69-1.57(m, 1H), 1.37(dq, J = 14.7, 7.4 Hz, 1H), 1.26(d, J = 25.5 |
| | Hz, 2H), 1.05-0.68(m, 2H). |
| Compound 100 | ¹HNMR(400 MHz, DMSO-d₆)δ7.74(dd, J = 9.1, 6.0 Hz, 1H), 7.31(t, J = 9.0 Hz, 1H), 7.06- |
| | 6.94(m, 2H), 5.59(d, J = 3.5 Hz, 2H), 5.37-5.18(m, 2H), 4.99(dd, J = 14.7, 5.8 Hz, 1H), |
| | 4.18(d, J = 1.8 Hz, 1H), 4.12-3.93(m, 4H), 3.84-3.71(m, 2H), 3.58-3.42(m, 2H), 3.08(d, J = |
| | 5.8 Hz, 2H), 3.03-2.93(m, 4H), 2.83(dd, J = 14.5, 8.2 Hz, 1H), 2.33-2.01(m, 4H), 2.00- |
| | 1.66(m, 9H). |
| Compound 102 | ¹HNMR(400 MHz, DMSO-d₆)δ7.78-7.69(m, 1H), 7.35-7.26(m, 1H), 7.05-6.93(m, 2 |
| | H), 5.58(s, 2H), 5.40-5.25(m, 1H), 5.27-5.18(m, 1H), 4.48-4.27(m, 1H), 4.26-4.15(m, 1 |
| | H), 4.15-3.89(m, 3H), 3.84-3.41(m, 3H), 3.25-2.76(m, 8H), 2.38-1.73(m, 7H), 1.28- |
| | 1.21(m, 3H). |
| Compound 104 | ¹HNMR(400 MHz, DMSO-d₆)δ7.78-7.69(m, 1H), 7.31(td, J = 9.0, 3.9 Hz, 1H), 7.06- |
| | 6.95(m, 2H), 5.59(s, 2H), 5.41-5.15(m, 2H), 4.52-4.35(m, 1H), 4.16-3.84(m, 6H), 3.69- |
| | 3.34(m, 3H), 3.17-2.96(m, 3H), 2.93-2.76(m, 1H), 2.74-2.52(m, 2H), 2.33-2.18(m, 1H), |
| | 2.16-2.08(m, 1H), 2.07-1.90(m, 4H), 1.90-1.68(m, 3H). |
| Compound | ¹HNMR(400 MHz, DMSO-d₆)δ7.80-7.60(m, 2H), 7.41-7.18(m, 1H), 7.04-6.98(m, 1 |
| 106B | H), 5.48(d, J = 90.6 Hz, 2H), 5.22(d, J = 10.5 Hz, 1H), 4.90(t, J = 15.1 Hz, 1H), 4.22(t, J = |
| | 6.6 Hz, 1H), 4.19-4.08(m, 1H), 4.02(d, J = 9.8 Hz, 1H), 3.87-3.67(m, 1H), 3.12(m, 2H), |
| | 2.94(dd, J = 10.5, 5.5 Hz, 3H), 2.86(s, 1H), 2.16(d, J = 20.7 Hz, 2H), 2.10-1.94(m, 4H), 1.94- |
| | 1.69(m, 5H), 1.69-1.54(m, 2H), 1.43-1.29(m, 1H). |
| Compound | ¹HNMR(400 MHz, DMSO-d₆)δ7.74(dd, J = 9.1, 6.0 Hz, 1H), 7.31(t, J = 9.0 Hz, 1H), 7.13- |
| 107A | 6.89(m, 2H), 5.59(d, J = 6.1 Hz, 2H), 5.34(s, 1H), 5.26-5.07(m, 2H), 4.16-3.91(m, 4H), |
| | 3.58-3.42(m, 1H), 3.29-3.15(m, 2H), 3.15-3.01(m, 2H), 3.01-2.89(m, 5H), 2.83(dd, J = |
| | 14.7, 8.4 Hz, 1H), 2.39-2.29(m, 1H), 2.22(ddd, J = 21.2, 13.7, 7.6 Hz, 1H), 2.12(d, J = 7.6 |
| | Hz, 1H), 2.02(dt, J = 12.3, 8.7 Hz, 2H), 1.95-1.66(m, 3H), 0.97(dd, J = 17.0, 6.6 Hz, 6H). |
| Compound | ¹HNMR(400 MHz, DMSO-d₆)δ7.74(dd, J = 9.1, 6.0 Hz, 1H), 7.31(t, J = 9.0 Hz, 1H), 7.13- |
| 107B | 6.89(m, 2H), 5.59(d, J = 6.1 Hz, 2H), 5.34(s, 1H), 5.26-5.07(m, 2H), 4.16-3.91(m, 4H), |
| | 3.58-3.42(m, 1H), 3.29-3.15(m, 2H), 3.15-3.01(m, 2H), 3.01-2.89(m, 5H), 2.83(dd, J = |
| | 14.7, 8.4 Hz, 1H), 2.39-2.29(m, 1H), 2.22(ddd, J = 21.2, 13.7, 7.6 Hz, 1H), 2.12(d, J = 7.6 |
| | Hz, 1H), 2.02(dt, J = 12.3, 8.7 Hz, 2H), 1.95-1.66(m, 3H), 0.97(dd, J = 17.0, 6.6 Hz, 6H). |
| Compound | ¹HNMR(400 MHz, DMSO-d₆)δ7.85-7.61(m, 1H), 7.31(td, J = 9.0, 5.1 Hz, 1H), 7.08- |
| 108A | 6.82(m, 2H), 5.99-5.82(m, 1H), 5.56(d, J = 30.9 Hz, 2H), 5.21(t, J = 37.8 Hz, 1H), 4.75(s, |
| | 1H), 4.10(dd, J = 16.0, 8.1 Hz, 1H), 3.98(dd, J = 10.2, 5.0 Hz, 1H), 3.89(s, 1H), 3.72(d, J = |
| | 17.4 Hz, 1H), 3.38(d, J = 14.8 Hz, 1H), 3.09(d, J = 13.5 Hz, 2H), 3.06-2.94(m, 3H), 2.83(d, |
| | J = 5.3 Hz, 1H), 2.11(d, J = 5.7 Hz, 1H), 2.02(d, J = 23.0 Hz, 2H), 1.90-1.69(m, 3H), 1.44- |
| | 1.33(m, 1H), 1.32-1.17(m, 4H). |
| Compound 109 | ¹HNMR(400 MHz, DMSO-d₆)δ7.74(dd, J = 9.1, 6.0 Hz, 1H), 7.31(td, J = 9.0, 3.4 Hz, 1H), |
| | 7.07-6.94(m, 2H), 5.58(s, 2H), 5.42-5.13(m, 2H), 4.53-4.38(m, 1H), 4.19-4.07(m, 1H), |
| | 4.04-3.90(m, 3H), 3.61-3.46(m, 1H), 3.46-3.35(m, 1H), 3.29-3.21(m, 1H), 3.16-3.05(m, |
| | 2H), 3.02(d, J = 9.9 Hz, 1H), 2.99(d, J = 5.4 Hz, 3H), 2.82(dd, J = 14.8, 8.3 Hz, 1H), 2.36- |
| | 2.17(m, 1H), 2.17-2.10(m, 1H), 2.06(d, J = 9.1 Hz, 1H), 1.99(s, 2H), 1.83(d, J = 15.2 Hz, 1 |
| | H), 1.80-1.70(m, 2H). |
| Compound 110 | ¹HNMR(400 MHz, DMSO-d₆)δ7.73(dd, J = 10.5, 4.3 Hz, 1H), 7.30(td, J = 9.0, 3.3 Hz, 1H), |
| | 6.98(t, J = 8.8 Hz, 2H), 5.58(d, J = 7.2 Hz, 2H), 5.28(d, J = 54.2 Hz, 1H), 5.05(d, J = 16.4 Hz, |
| | 1H), 4.83(dd, J = 53.4, 4.8 Hz, 1H), 4.35(s, 1H), 4.23-4.01(m, 2H), 3.95(d, J = 10.2 Hz, 1H), |
| | 3.22-3.07(m, 3H), 2.98(d, J = 16.5 Hz, 4H), 2.83(s, 1H), 2.07(dd, J = 43.8, 17.9 Hz, 5H), |
| | 1.91-1.58(m, 7H), 1.53(s, 1H). |
| Compound 112 | ¹HNMR(400 MHz, DMSO-d₆)δ7.88-7.64(m, 1H), 7.31(t, J = 9.0 Hz, 1H), 7.14-6.94(m, |
| | 2H), 6.05(d, J = 5.3 Hz, 2H), 5.77(s, 1H), 5.60(s, 2H), 5.27(d, J = 53.5 Hz, 1H), 5.01(t, J = |
| | 9.0 Hz, 1H), 4.24-3.90(m, 3H), 3.13-2.96(m, 6H), 2.84(dd, J = 13.5, 7.7 Hz, 3H), 2.17-1.94 |
| | (m, 4H), 1.93-1.71(m, 3H). |
| Compound 119 | ¹HNMR(400 MHz, DMSO-d₆)δ10.99-10.62(m, 1H), 10.26(s, 1H), 7.92(dd, J = 9.2, 5.7 |
| | Hz, 1H), 7.54(t, J = 8.9 Hz, 1H), 7.44-7.37(m, 1H), 7.23-7.13(m, 1H), 5.58(d, J = 52.4 Hz, |
| | 2H), 5.33-5.20(m, 1H), 4.63-4.44(m, 2H), 4.15-3.98(m, 2H), 3.97-3.64(m, 5H), 3.61- |
| | 3.44(m, 1H), 3.39-3.17(m, 2H), 3.11-2.92(m, 3H), 2.39-2.26(m, 1H), 2.25-1.97(m, 4H), |
| | 1.82-1.67(m, 3H). |
| Compound 120 | ¹HNMR(400 MHz, DMSO-d₆)δ10.99-10.53(m, 1H), 7.82-7.74(m, 1H), 7.38-7.29(m, |
| | 1H), 7.11-6.94(m, 2H), 5.63(s, 1H), 5.51(s, 1H), 5.04-4.92(m, 1H), 4.65-4.46(m, 2H), |
| | 4.46-4.33(m, 1H), 4.09-3.97(m, 1H), 3.93-3.60(m, 5H), 3.43-3.21(m, 3H), 3.17-3.00(m, |
| | 3H), 2.39-2.23(m, 2H), 2.23-2.09(m, 2H), 2.09-1.86(m, 3H), 1.22-1.13(m, 3H). |
| Compound 121 | ¹HNMR(400 MHz, DMSO-d₆)δ10.86(s, 1H), 7.89-7.69(m, 1H), 7.33(tt, J = 26.4, 13.3 |
| | Hz, 1H), 7.11(d, J = 16.2 Hz, 1H), 6.99(s, 1H), 5.63(s, 1H), 5.50(s, 1H), 5.37-5.28(m, 1H), |
| | 4.96(d, J = 9.8 Hz, 2H), 4.64-4.48(m, 2H), 4.48-4.29(m, 1H), 4.04-3.68(m, 4H), 3.36- |
| | 3.22(m, 2H), 3.18-2.97(m, 3H), 2.68-2.52(m, 1H), 2.47(s, 1H), 2.31(d, J = 8.9 Hz, 1H), |
| | 2.16(ddd, J = 19.3, 13.4, 6.1 Hz, 3H), 2.05(s, 1H), 1.86-1.49(m, 3H), 1.49-1.15(m, 2H), 0.84 |
| | (ddd, J = 52.3, 28.2, 6.9 Hz, 3H). |
| Compound 125 | ¹HNMR(400 MHz, DMSO-d₆)δ7.85-7.62(m, 1H), 7.33(td, J = 9.1, 4.7 Hz, 1H), 7.01(dd, |
| | J = 17.0, 14.8 Hz, 2H), 5.32(ddd, J = 10.3, 6.7, 3.8 Hz, 2H), 5.22(d, J = 6.6 Hz, 2H), 4.62(dd, |
| | J = 12.0, 5.7 Hz, 1H), 4.54(dd, J = 12.0, 4.6 Hz, 1H), 4.45(ddd, J = 18.2, 11.3, 7.4 Hz, 1H), |
| | 4.27-4.10(m, 2H), 4.01(ddd, J = 13.3, 10.8, 4.2 Hz, 2H), 3.94(s, 1H), 3.90(d, J = 3.7 Hz, 1H), |
| | 3.69-3.47(m, 2H), 3.45-3.24(m, 2H), 3.18(d, J = 6.4 Hz, 1H), 3.00(t, J = 6.9 Hz, 3H), 2.91 |
| | (dd, J = 16.0, 6.9 Hz, 1H), 2.78-2.66(m, 1H), 2.27(dd, J = 22.1, 16.1 Hz, 2H), 2.19-2.06(m, |
| | 1H), 2.06-1.89(m, 3H). |
| Compound 127 | ¹HNMR(400 MHz, DMSO-d₆-d₆)δ7.71-7.63(m, 1H), 7.33-7.18(m, 2H), 7.02-6.91(m, |
| | 1H), 5.58(d, J = 52.2 Hz, 1H), 5.35-5.21(m, 1H), 4.63-4.45(m, 2H), 4.18-4.00(m, 2H), |
| | 3.89(d, J = 12.0 Hz, 2H), 3.84-3.66(m, 3H), 3.57-3.39(m, 1H), 3.38-3.16(m, 2H), 3.01(d, |
| | J = 2.9 Hz, 3H), 2.68-2.52(m, 1H), 2.49-2.43(m, 2H), 2.43-2.29(m, 3H), 2.29-2.15(m, 2 |
| | H), 2.15-1.89(m, 3H), 1.75(t, J = 6.6 Hz, 3H), 0.91-0.75(m, 3H). |

### Pharmacological Experiments

### 1. SOS1 Catalyzed Nucleotide Exchange Assay

The inhibition activity of each of compounds on GDP form K-Ras was evaluated by SOS1 catalyzed nucleotide exchange assays. K-Ras G12D and K-Ras G12V proteins were used in this assay.

K-Ras (His tag, as 1-169) pre-loaded with GDP was pre-incubated with each of compounds in the presence of 10 nM GDP in a 384-well plate (Greiner) for 15 mins, purified SOS1 ExD (Flag tag, aa 564-1049), BODIPY™ FL GTP (Invitrogen) and monoclonal antibody anti 6HIS—Tb cryptate Gold (Cisbio) were added to the assay wells and incubated for 4 hours at 25° C. Final concentration for each component in assay wells is shown in Table 1. Wells containing the same percent of DMSO served as vehicle control, and wells without K-Ras served as low control. TR-FRET signals were read on Tecan Spark multimode microplate reader. The parameters were F486: Excitation 340 nm, Emission 486 nm, Lag time 100 μs, Integration time 200 μs; F515: Excitation 340 nm, Emission 515 nm, Lag time 100 μs, Integration time 200 μs. TR-FRET ratios for each individual wells were calculated by equation: TR-FRET ratio=(Signal F515/Signal F486)*10000. The percent of activation of compounds treated wells were normalized between vehicle control and negative control (% Activation=(TR-FRET ratio_{Compound treated}−TR-FRET ratio_{Negative control})/(TR-FRET ratio_{Vehicle control}−TR-FRET ratio_{Negative control})*100%). The data were analyzed either by fitting a 4-parameter logistic model or b Excel to calculate IC₅₀ values. The resulted are shown in the following Table 3.

TABLE 1

| | | | | | MAb Anti |
|---|---|---|---|---|---|
| | | | BODIPY ™ | SOS1 | 6HIS-Tb |
| KRAS | KRAS | GDP | FL GTP | ExD | cryptate |
| proteins | Conc. | Conc. | conc. | Conc. | Gold Conc. |
| GDP-KRAS | 1.5 nM | 5 nM | 80 nM | 0.5 μM | 52.5 ng/mL |
| G12D | | | | | |
| GDP-KRAS | 1.5 nM | 5 nM | 80 nM | 0.5 μM | 52.5 ng/mL |
| G12V | | | | | |

2. GTP-K-Ras and cRAF Interaction Assay

The inhibition activity of each of compounds on GTP form K-Ras was evaluated by GppNp-K-Ras and cRAF interaction assays. GppNp is an analog of GTP. K-Ras G12D and K-Ras G12V proteins were used in this assay.

K-Ras (His tag, as 1-169) pre-loaded with GppNp was pre-incubated with each of compounds in the presence of 200 μM GTP in a 384-well plate (Greiner) for 15 mins, cRAF RBD (GST tag, aa 50-132, CreativeBioMart), monoclonal antibody anti GST-d2 (Cisbio) and monoclonal antibody anti 6HIS—Tb cryptate Gold (Cisbio) were added to the assay wells and incubated for 2 hours at 25° C. Final concentration for each component in assay wells is shown in Table 2. Wells containing same percent of DMSO served as vehicle control, and wells without K-Ras served as negative control. HTRF signals were read on Tecan Spark multimode microplate reader and HTRF ratios were calculated under manufacturer's instructions. The percent of activation of compounds treated wells were normalized between vehicle control and negative control (% Activation=(HTRF ratio_{Compound treated}−HTRF ratio_{Negative control})/(HTRF ratio_{Vehicle control}−HTRF ratio_{Negative control})*100%). The data were analyzed either by fitting a 4-parameter logistic model or by Excel to calculate IC₅₀ values. The resulted are shown in the following Table 3.

TABLE 2

| | | | | MAb Anti | |
|---|---|---|---|---|---|
| | | cRAF | | 6HIS-Tb | MAb Anti |
| | KRAS | RBD | GTP | cryptate | GST-d2 |
| KRAS proteins | Conc. | Conc. | Conc. | Gold Conc. | Conc. |
| GppNp-KRAS | 5 nM | 35 nM | 100 μM | 52.5 ng/mL | 1 μg/mL |
| G12D | | | | | |
| GppNp-KRAS | 8 nM | 35 nM | 100 μM | 52.5 ng/mL | 1 μg/mL |
| G12V | | | | | |

TABLE 3

| | Biochemical assays, IC₅₀ (nM) | | | |
|---|---|---|---|---|
| | KRAS G12D | | KRAS G12V | |
| Compound | GDP | GppNp | GDP | GppNp |
| Compound 1 | 0.724 | 91.0 | 0.606 | 157 |
| Compound 2 | 1.69 | 195 | 1.19 | 252 |
| Compound 3 | 2.95 | 415 | 1.88 | 660 |
| Compound 4 | 2.58 | 164 | 2.04 | 292 |
| Compound 5A | 1.82 | 104 | 1.12 | 130 |
| Compound 5B | 1.62 | 80.8 | 0.860 | 161 |
| Compound 6A | 1.24 | 497 | 3.37 | |
| Compound 6B | 3.83 | 808 | 3.82 | |
| Compound 7 | 0.555 | 29.5 | 0.282 | 58.6 |
| Compound 8 | 1.03 | 66.4 | 0.550 | 200 |
| Compound 9 | 2.74 | 14.1 | 1.54 | 18.4 |
| Compound 10 | 2.51 | 57.1 | 2.01 | 54.9 |
| Compound 11 | 3.49 | 172 | 2.62 | 366 |
| Compound 11B | 5.69 | 233 | 3.92 | 311 |
| Compound 10B | 0.766 | 44.2 | 0.538 | 52.1 |
| Compound 12 | 12.8 | | 5.57 | |
| Compound 13 | 1.21 | 90.6 | 0.978 | 187 |
| Compound 14B | 1.67 | 98.5 | 1.96 | 153 |
| Compound 15 | 1.98 | 391 | 2.26 | >1000 |
| Compound 16 | 29.7 | 651 | 15.2 | >1000 |
| Compound 18A | 5.42 | | 4.72 | |
| Compound 19 | 0.746 | | 0.534 | |
| Compound 20 | 1.12 | 229 | 1.53 | 534 |
| Compound 21 | 0.767 | 82.4 | 0.53 | 103 |
| Compound 22 | 6.97 | | 5.16 | |
| Compound 23 | 30.0 | | 36.2 | |
| Compound 25 | 2.58 | 306 | 2.97 | 1000 |
| Compound 26A | 2.30 | 109 | 2.57 | 220 |
| Compound 26B | 0.551 | 63.2 | 0.839 | 120 |
| Compound 27A | 47.8 | >1000 | 65.1 | >1000 |
| Compound 27B | 2.51 | 174 | 2.69 | 563 |
| Compound 28 | 13.0 | | 18.1 | |
| Compound 29 | 29.5 | | 39.5 | |
| Compound 30 | 10.3 | 191 | 9.03 | 466 |
| Compound 32 | 3.69 | | 2.10 | |
| Compound 33 | 0.866 | 99.9 | 0.890 | 223 |
| Compound 34 | 1.31 | 50.0 | 0.798 | 68.1 |
| Compound 35 | 3.53 | 155 | 2.78 | 637 |
| Compound 36 | 0.339 | 19.70 | 0.189 | 30.4 |
| Compound 37 | 0.867 | 42.0 | 0.535 | 62.1 |
| Compound 38A | 0.460 | 51.9 | 0.292 | 75.6 |
| Compound 38B | 1.05 | 174 | 0.397 | 685 |
| Compound 39A | 1.01 | 46.1 | 0.667 | 142 |
| Compound 39B | 1.80 | 189 | 1.22 | 226 |
| Compound 40A | 0.875 | 9.42 | 0.912 | 36.4 |
| Compound 40B | 1.55 | 162 | 7.01 | 705 |
| Compound 41 | 5.51 | 376 | 4.31 | >1000 |
| Compound 42 | 1.09 | 23.3 | 0.801 | 26.4 |
| Compound 43 | 185 | >1000 | 98.5 | >1000 |
| Compound 44 | 2.07 | 95.6 | 0.635 | 208 |
| Compound 45 | 0.796 | 63.8 | 0.319 | 85.3 |
| Compound 46 | 3.28 | 69.8 | 1.97 | 107 |
| Compound 47 | 1.25 | 71.8 | 0.945 | 163 |
| Compound 48 | 0.759 | 35.8 | 0.595 | 55.4 |
| Compound 49 | 2.10 | 48.1 | 3.38 | 99.8 |
| Compound 50 | 0.750 | 3.53 | 0.697 | 4.37 |
| Compound 51 | 0.792 | 64.4 | 0.842 | 103 |
| Compound 52 | 2.83 | 146 | 1.34 | 363 |
| Compound 52B | 3.25 | 302 | 1.97 | 622 |
| Compound 53 | 6.82 | 989 | 7.14 | >1000 |
| Compound 54A | 341 | >1000 | 40.2 | >1000 |
| Compound 54B | 1.09 | 29.3 | 1.08 | 123 |
| Compound 55 | 2.49 | 93.9 | 3.38 | 290 |
| Compound 56B | 2.10 | 63.9 | 1.45 | 158 |
| Compound 57 | 0.536 | 29.2 | 0.279 | 59.2 |
| Compound 58 | 1.04 | 34.0 | 0.830 | 124 |
| Compound 59 | 1.77 | 101 | 1.02 | 160 |
| Compound 60 | 1.55 | 140 | 0.597 | 113 |
| Compound 61 | 1.14 | 24.2 | 1.33 | 114 |
| Compound 62A | 703 | >1000 | 389 | >1000 |
| Compound 62B | 0.548 | 21.6 | 0.887 | 63.7 |
| Compound 63 | 0.839 | 7.27 | 0.769 | 10.4 |
| Compound 64 | 0.367 | 75.7 | 0.259 | 75.1 |
| Compound 65 | 2.08 | 230 | 0.601 | 270 |
| Compound 66A | 169 | | 331 | |
| Compound 66B | 0.983 | 4.06 | 0.924 | 8.62 |
| Compound 67 | 1.72 | 59.9 | 1.36 | 113 |
| Compound 68 | 2.18 | 187 | 3.49 | 356 |
| Compound 69 | 3.56 | 302 | 3.18 | 265 |
| Compound 70 | 2.07 | 426 | 1.90 | 1000 |
| Compound 71 | 1.55 | 1000 | 2.23 | >1000 |
| Compound 72 | 3.17 | 105 | 3.50 | 257 |
| Compound 73 | 1.49 | 140 | 1.02 | 503 |
| Compound 74 | 3.40 | 243 | 2.07 | 344 |
| Compound 75 | 5.62 | 386 | 4.00 | 880 |
| Compound 76 | 2.84 | 96.3 | 1.14 | 118 |
| Compound 77 | 7.63 | 177 | 15.1 | 1000 |
| Compound 78 | 21.3 | >1000 | 10.0 | >1000 |
| Compound 79 | 3.98 | 240 | 3.00 | 542 |
| Compound 80 | 10.6 | 314 | 12.2 | >1000 |
| Compound 81 | 78.8 | >1000 | 89.3 | >1000 |
| Compound 82 | 3.63 | >1000 | 11.6 | >1000 |
| Compound 83 | 0.712 | 23.1 | 1.45 | 104 |
| Compound 84 | 5.29 | 115 | 2.59 | 146 |
| Compound 85 | 8.56 | >1000 | 3.81 | >1000 |
| Compound 86 | 10.3 | >1000 | 9.94 | >1000 |
| Compound 87 | 33.3 | 882 | 29.9 | >1000 |
| Compound 88 | 0.643 | 23.5 | 0.579 | 120 |
| Compound 89 | 0.641 | 5.93 | 0.495 | 23.8 |
| Compound 90 | 3.72 | 190 | 1.28 | 898 |
| Compound 91A | 2.02 | 114 | 1.81 | 427 |
| Compound 91B | 104 | >1000 | 108 | >1000 |
| Compound 92A | 9.92 | 341 | 3.50 | 686 |
| Compound 92B | 3.08 | 339 | 1.70 | >1000 |
| Compound 93A | 11.1 | 593 | 21.3 | >1000 |
| Compound 94 | 1.17 | 224 | 0.576 | 204 |
| Compound 95 | 0.561 | 8.34 | 0.727 | 27.3 |
| Compound 96 | 18.7 | >1000 | 10.2 | >1000 |
| Compound 97 | 3.42 | 86.6 | 3.88 | 654 |
| Compound 99 | 7.13 | 262 | 3.48 | 735 |
| Compound 100 | 9.91 | 647 | 3.78 | 1000 |
| Compound 101 | 8.39 | 1000 | 5.64 | >1000 |
| Compound 102 | 7.13 | 136 | 3.04 | 177 |
| Compound 103 | 3.45 | 214 | 1.79 | 101 |
| Compound 104 | 9.19 | >1000 | 17.6 | >1000 |
| Compound 105 | 44.1 | >1000 | 19.5 | >1000 |
| Compound 106A | 630 | >1000 | 226 | >1000 |
| Compound 106B | 1.45 | 47.4 | 1.45 | 225 |
| Compound 107A | 34.4 | >1000 | 14.6 | >1000 |
| Compound 108B | 142 | >1000 | 63.9 | >1000 |
| Compound 108A | 3.32 | 106 | 2.95 | 214 |
| Compound 109 | 0.921 | 34.4 | 0.851 | 56.9 |
| Compound 110 | 2.87 | 244 | 2.97 | >1000 |
| Compound 111 | 8.97 | >1000 | 4.93 | >1000 |
| Compound 112 | 33.4 | 921 | 21.6 | >1000 |
| Compound 113 | 4.62 | 623 | 3.81 | 513 |
| Compound 114 | 3.73 | 295 | 2.08 | 747 |
| Compound 115 | 2.56 | 67.4 | 3.55 | 316 |
| Compound 116 | 6.76 | 65.8 | 3.55 | 203 |
| Compound 117 | 5.88 | 702 | 3.37 | 884 |
| Compound 118 | 11.2 | 1000 | 11.7 | >1000 |
| Compound 119 | 1.53 | 71.1 | 0.815 | 233 |
| Compound 120 | 1.96 | 134 | 1.98 | 467 |
| Compound 121 | 2.94 | 170 | 3.89 | 707 |
| Compound 122 | 2.68 | 174 | 1.39 | 169 |
| Compound 123B | 13.9 | 358 | 4.68 | >1000 |
| Compound 123A | 217 | >1000 | 71.3 | >1000 |
| Compound 123D | 0.994 | 54.3 | 0.857 | 209 |
| Compound 123C | 169 | >1000 | 114 | >1000 |
| Compound 124 | 8.98 | 403 | 6.68 | >1000 |
| Compound 125 | 1.34 | 25.0 | 0.664 | 34.3 |
| Compound 126 | 5.04 | 255 | 5.45 | >1000 |
| Compound 127 | 3.87 | 299 | 2.13 | >1000 |
| Compound 132 | 3.39 | 78.4 | 3.01 | 224 |
| Compound 134 | 1.75 | 21.6 | 1.44 | 50.4 |
| Compound 135 | 1.25 | 55.8 | 1.07 | 88.8 |
| Compound 136 | 115 | >1000 | 101 | >1000 |
| Compound 137 | 1.86 | 210 | 1.84 | 486 |
| Compound 138 | 2.51 | 150 | 1.52 | 469 |
| Compound 139 | 19.6 | 1000 | 21.6 | >1000 |
| Compound 140 | 13.8 | >1000 | 7.11 | >1000 |
| Compound 142 | 233 | >1000 | 65.4 | >1000 |
| Compound 143 | 2.51 | 246 | 2.94 | 631 |
| Compound 144 | 1.14 | 11.2 | 1.41 | 26.2 |
| Compound 145 | 2.55 | >1000 | 4.63 | >1000 |
| Compound 148 | 287 | >1000 | 403 | >1000 |
| Compound 149 | 10.2 | >1000 | 5.08 | >1000 |
| Compound 150 | 8.27 | >1000 | 7.35 | >1000 |
| Compound 153 | 83.4 | >1000 | 13.7 | >1000 |
| Compound 154 | 3.19 | 400 | 2.38 | 543 |
| Compound 155A | 161 | >1000 | 200 | >1000 |
| Compound 155B | 1.86 | 79.9 | 2.08 | 148 |
| Compound 156A | 3.40 | 126 | 1.98 | 440 |
| Compound 156B | 63.8 | >1000 | 37.8 | >1000 |
| Compound 156C | 1.82 | 32.7 | 2.15 | 336 |
| Compound 158A | 0.996 | 10.8 | 0.939 | 4.93 |
| Compound 159A | 8.75 | >1000 | 5.89 | >1000 |
| Compound 159B | 4.39 | 597 | 2.84 | 784 |
| Compound 160 | 2.36 | 74.8 | 3.31 | 220 |
| Compound 161 | 1.77 | 53.8 | 0.901 | 101 |
| Compound 162 | 8.80 | 928 | 5.73 | >1000 |
| Compound 163A | 301 | >1000 | 423 | >1000 |
| Compound 163B | 0.879 | 8.04 | 1.04 | 18.3 |
| Compound 164 | 4.68 | 274 | 4.18 | 750 |
| Compound 165 | 10.4 | >1000 | 9.27 | >1000 |
| Compound 166 | 1.15 | 74.2 | 1.55 | 146 |
| Compound 167 | 8.54 | >1000 | 9.32 | >1000 |
| Compound 168 | 503 | >1000 | 631 | >1000 |
| Compound 169 | 1.67 | 164 | 3.33 | 245 |
| Compound 170 | 8.14 | >1000 | 2.03 | >1000 |
| Compound 171 | 5.36 | 198 | 3.58 | 164 |
| Compound 172 | 12.9 | >1000 | 1.04 | >1000 |
| Compound 173 | 1.27 | 56.8 | 1.63 | 79.3 |
| Compound 174 | 67.6 | >1000 | 39.8 | >1000 |
| Compound 175 | 1.14 | 61.7 | 1.28 | 79.2 |
| Compound 176A | 408 | >1000 | 473 | >1000 |
| Compound 176B | 1.16 | 7.44 | 1.01 | 18.1 |

### 3. Phospho-ERK1/2(THR202/TYR204) HTRF Assay

p-ERK (MAPK pathway) inhibition activity of each of compounds in K-Ras G12D and K-Ras G12V cell lines indicated in Table 4 was evaluated.

TABLE 4

| | | Cell seeding | | | |
|---|---|---|---|---|---|
| | K-Ras | density, | | | |
| Cell lines | mutation | cells/well | Culture medium | Assay medium | Cell incubator |
| AGS | G12D | 60000 | F-12K, 10% FBS | F-12K, 0.1% | 37° C., 5% CO₂ |
| | | | | FBS | |
| SW620 | G12V | 60000 | L-15, 10% FBS | L-15, 0.1% FBS | 37° C., 100% Air |
| Capan-2 | G12V | 30000 | McCoy's 5a, | McCoy's 5a, | 37° C., 5% CO₂ |
| | | | 10% FBS | 0.1% FBS | |

Each of cells in culture medium was seeded in 96-well plates at density indicated in Table 4 and put in a cell incubator to incubate overnight. The next day, the culture medium was removed and the compound diluted in assay medium was added in each well. After 2 hours incubation in a cell incubator, the assay medium in 96-well plates was removed, 50 μL of 1× blocking reagent-supplemented lysis buffer (Cisbio) was added and the plates were incubated at 25° C. for 45 min with shaking. 10 μL of cell lysates from the 96-well plates were transferred to a 384-well plate (Greiner) containing 2.5 μL/well HTRF® pre-mixed antibodies (Cisbio 64AERPEH). The plate was incubated 4 hours at 25° C. and read HTRF signals on Tecan Spark multimode microplate reader. The data were analyzed using a 4-parameter logistic model to calculate IC₅₀ values. The resulted are shown in the following Table 5:

TABLE 5

| | pERK, IC₅₀, nM | | |
|---|---|---|---|
| Compound | AGS | SW620 | Capan-2 |
| Compound 1 | 3.04 | 4.43 | 6.25 |
| Compound 2 | 10.3 | 12.1 | |
| Compound 3 | 8.60 | 1.39 | |
| Compound 4 | 11.2 | | 3.85 |
| Compound 5 | 1.79 | 4.15 | |
| Compound 6A | 6.86 | 12.5 | |
| Compound 6B | 12.2 | 16.2 | |
| Compound 7 | 0.849 | 1.55 | |
| Compound 8 | 2.54 | 2.23 | |
| Compound 9 | 1.11 | 0.724 | |
| Compound 10 | 1.13 | 3.54 | |
| Compound 10B | 0.788 | 1.66 | 0.861 |
| Compound 11 | 219 | 63.6 | |
| Compound 11B | 94.2 | 53.5 | |
| Compound 12 | 21.8 | 34.4 | |
| Compound 13 | 4.45 | 2.27 | |
| Compound 14B | 1.03 | 5.71 | |
| Compound 15 | 7.73 | 29.4 | |
| Compound 18A | 13.9 | 59.4 | |
| Compound 19 | 37.1 | 3.85 | |
| Compound 20 | 15.3 | | 10.3 |
| Compound 21 | 2.24 | 4.04 | |
| Compound 22 | 107 | 30.8 | |
| Compound 25 | 19.5 | 32.5 | |
| Compound 26A | 4.97 | 6.99 | |
| Compound 26B | 2.50 | 2.05 | |
| Compound 27B | 6.96 | 12.4 | |
| Compound 32 | 22.7 | 22.7 | |
| Compound 33 | 0.643 | 2.83 | |
| Compound 34 | 1.65 | 2.00 | |
| Compound 35 | 17.1 | 8.01 | |
| Compound 36 | 0.582 | 1.01 | 0.352 |
| Compound 37 | 3.31 | 1.84 | |
| Compound 38A | 0.971 | 0.648 | |
| Compound 38B | 2.32 | 2.59 | |
| Compound 39A | 1.64 | 1.93 | |
| Compound 39B | 2.89 | 4.98 | |
| Compound 40A | 0.571 | 0.328 | 0.843 |
| Compound 41 | 72.7 | | |
| Compound 42 | 1.69 | 2.09 | |
| Compound 44 | 53.8 | 51.4 | |
| Compound 45 | 0.865 | 3.59 | |
| Compound 46 | 10.9 | 8.14 | |
| Compound 47 | 1.68 | 6.26 | |
| Compound 48 | 3.53 | 3.68 | 1.65 |
| Compound 49 | 4.89 | 13.3 | |
| Compound 50 | 0.333 | 0.678 | |
| Compound 51 | 1.59 | 2.33 | |
| Compound 52B | 16.0 | | 2.01 |
| Compound 53 | 88.7 | 46.9 | |
| Compound 54B | 4.16 | 1.84 | |
| Compound 55 | 1.56 | 3.50 | |
| Compound 56B | 6.83 | 5.23 | |
| Compound 57 | 1.46 | | 0.453 |
| Compound 58 | 1.43 | | 0.404 |
| Compound 59A | 3.73 | | 0.316 |
| Compound 60 | 4.58 | | 1.56 |
| Compound 61 | 1.65 | | 1.52 |
| Compound 62B | 1.25 | | 1.95 |
| Compound 63 | 0.664 | 0.571 | |
| Compound 64 | 1.99 | | 0.785 |
| Compound 65 | 2.52 | 6.11 | |
| Compound 66B | 2.27 | — | 1.94 |
| Compound 67 | 20.9 | 8.74 | |
| Compound 68 | 3.29 | 5.95 | |
| Compound 69 | 26.3 | 5.63 | |
| Compound 70 | 42.6 | 50.9 | |
| Compound 71 | 8.97 | 22.7 | |
| Compound 72 | 27.3 | | 10.5 |
| Compound 73 | 11.7 | | 5.6 |
| Compound 74 | 16.7 | | 2.24 |
| Compound 75 | 105 | | 23.4 |
| Compound 76 | 2.82 | 2.08 | |
| Compound 77 | 6.88 | | |
| Compound 79 | 8.24 | | 18.5 |
| Compound 82 | 86.9 | | 19.9 |
| Compound 83 | 1.25 | | 2.66 |
| Compound 84 | 92.8 | | 12.4 |
| Compound 85 | 108 | | 108 |
| Compound 88 | 1.35 | | 2.98 |
| Compound 89 | 0.624 | | 0.886 |
| Compound 90 | 9.43 | 9.38 | |
| Compound 91A | 16.2 | 6.39 | |
| Compound 92A | 52.0 | | 27.2 |
| Compound 92B | 26.3 | | 4.35 |
| Compound 94 | 4.58 | 1.71 | |
| Compound 95 | 0.649 | | 1.38 |
| Compound 96 | | 46.6 | |
| Compound 97 | 43.8 | 65.4 | |
| Compound 99 | 17.7 | | 19.0 |
| Compound 100 | 51.2 | | 5.87 |
| Compound 101 | 48.3 | | 25.0 |
| Compound 102 | 22.1 | 11.4 | |
| Compound 103 | 9.29 | 10.1 | |
| Compound 104 | 485 | | |
| Compound 106B | 2.98 | | 1.48 |
| Compound 108A | 7.82 | | 8.81 |
| Compound 109 | 2.02 | | 0.885 |
| Compound 110 | 6.05 | | 17.5 |
| Compound 111 | 52.1 | | 19.4 |
| Compound 113 | 33.6 | 21.4 | |
| Compound 114 | 42.5 | 28.3 | |
| Compound 115 | 3.40 | | 5.16 |
| Compound 116 | 20.8 | | 5.97 |
| Compound 117 | 65.1 | | 18.7 |
| Compound 119 | 16.8 | 13.0 | |
| Compound 120 | 22.2 | | 5.60 |
| Compound 121 | 9.25 | | 8.38 |
| Compound 122 | 7.53 | | 2.10 |
| Compound 123D | 2.18 | | 2.07 |
| Compound 124 | 19.7 | | 15.0 |
| Compound 125 | 0.764 | | 0.905 |
| Compound 126 | 42.1 | | 66.2 |
| Compound 127 | 34.0 | 21.9 | |
| Compound 128 | 2.45 | | 1.57 |
| Compound 132 | | | 154 |
| Compound 138 | 26.7 | | 5.27 |
| Compound 149 | | | 77.8 |
| Compound 150 | 50.9 | | 54.1 |
| Compound 154 | 23.0 | | 7.45 |
| Compound 155B | 4.62 | | 4.72 |
| Compound 156A | 14.9 | | 2.96 |
| Compound 156C | 5.00 | | 8.41 |
| Compound 158 | 15.5 | | 22.8 |
| Compound 159A | 102 | | 8.54 |
| Compound 159B | 33.2 | | 5.40 |
| Compound 160 | 39.9 | | 62.0 |
| Compound 161 | 34.8 | | 48.4 |
| Compound 162 | 76.1 | | 18.1 |
| Compound 163B | 6.34 | | 6.20 |
| Compound 164 | 106 | | 33.3 |
| Compound 165 | 87.9 | | 32.0 |
| Compound 166 | 4.92 | | 2.62 |
| Compound 167 | 101 | | 51.9 |
| Compound 169 | 10.2 | | 0.778 |
| Compound 170 | 91.6 | | 29.6 |
| Compound 171 | 28.7 | | 2.54 |
| Compound 172 | | | 56.4 |
| Compound 173 | 3.95 | | 1.41 |
| Compound 175 | 2.41 | | 0.784 |
| Compound 176B | 219 | | 203 |

### 4. Cell Growth Inhibition Assay

The cell growth inhibition activity of each of compounds was tested by performing cell growth inhibition assays on K-Ras G12D and K-Ras G12V cell lines indicated in Table 6.

TABLE 6

| | | Cell seeding | | | |
|---|---|---|---|---|---|
| | K-Ras | density, | | Assay | |
| Cell lines | mutation | cells/well | Culture medium | format | Cell incubator |
| AGS | G12D | 500 | F-12K, 10% FBS | 2D | 37° C., 5% CO₂ |
| AsPC-1 | G12D | 1000 | RPMI 1640, 10% FBS | 2D | 37° C., 5% CO₂ |
| SW620 | G12V | 1500 | L-15, 10% FBS | 2D | 37° C., 100% Air |

### 2D Cell Growth Inhibition Assays

Each of cells in culture medium was plated in TC-treated 96-well plates at a density indicated in Table 4 and incubated in a cell incubator overnight. The next day, each of compounds was diluted in culture medium and added to the plates. After 6 days incubation in cell incubator, the cell viability was detected by CellTiter-Glo® Cell Viability Assay kit (Promega). Luminescent signals were read on Tecan Spark multimode microplate reader and analyzed using a 4-parameter logistic model to calculate absolute IC₅₀ values. The resulted are shown in the following Table 5.

TABLE 5

Cell viability, IC₅₀, nM

| Compound | AGS | AsPC-1 | SW620 |
|---|---|---|---|
| Compound 1 | 5.05 | 102 | 9.37 |
| Compound 2 | 13.5 | 386 | 44.1 |
| Compound 3 | 47.2 | 180 | 25.5 |
| Compound4 | 18.2 | 151 | 20.9 |
| Compound 5 | 9.12 | 61.6 | 5.78 |
| Compound 6A | 33.0 | 175 | 26.4 |
| Compound 6B | 52.2 | 266 | 28.3 |
| Compound 7 | 3.23 | 35.6 | 2.53 |
| Compound 8 | 5.83 | 79.4 | 5.84 |
| Compound 9 | 2.09 | 12.5 | 0.658 |
| Compound 10 | 6.93 | 44.6 | 2.60 |
| Compound10A | 1323 | >10000 | >1000 |
| Compound 10B | 2.03 | 18.5 | 1.47 |
| Compound11 | 327 | 583 | 93.4 |
| Compound 11B | 204 | 806 | 117 |
| Compound 12 | 94.7 | 360 | 32.9 |
| Compound 13 | 19.6 | 47.4 | 7.48 |
| Compound 14B | 4.92 | 46.0 | 10.0 |
| Compound 15 | 14.6 | 620 | 158 |
| Compound 18A | 101 | 391 | 62.0 |
| Compound 19 | 26.3 | 294 | 10.5 |
| Compound 20 | 22.1 | 110 | 24.4 |
| Compound 21 | 8.57 | 43.5 | 4.88 |
| Compound 22 | 142 | 470 | 59.5 |
| Compound 25 | 51.6 | 232 | 29.7 |
| Compound 26A | 43.3 | 158 | 13.9 |
| Compound 26B | 6.70 | 54.2 | 10.6 |
| Compound 27B | 30.4 | 110 | 16.9 |
| Compound 29 | | 620 | 158 |
| Compound 30 | | | 436 |
| Compound 32 | 43.1 | 871 | 60.3 |
| Compound 33 | 9.70 | 66.4 | 10.9 |
| Compound 34 | 2.05 | 36.4 | 3.49 |
| Compound 35 | 38.0 | 667 | 17.7 |
| Compound 36 | 1.21 | 16.9 | 1.20 |
| Compound 37 | 4.79 | 27.1 | 5.07 |
| Compound 38A | 1.51 | 28.7 | 2.64 |
| Compound 38B | 10.2 | 61.3 | 5.47 |
| Compound 39A | 6.27 | 48.7 | 3.38 |
| Compound 39B | 8.76 | 139 | 16.6 |
| Compound 40A | 0.963 | 2.54 | 2.71 |
| Compound 41 | 301 | 1139 | 40.1 |
| Compound 42 | 4.71 | 39.3 | 2.79 |
| Compound 44 | 178 | 423 | 38.6 |
| Compound 45 | 5.58 | 22.9 | 9.38 |
| Compound 46 | 77.6 | >10000 | 13.4 |
| Compound 47 | 7.82 | 39.9 | 7.33 |
| Compound 48 | 3.44 | 26.6 | 2.55 |
| Compound 49 | 5.45 | 29.9 | 4.68 |
| Compound 50 | 1.04 | 11.2 | 0.326 |
| Compound 51 | 4.53 | 34.1 | 4.73 |
| Compound 52B | 21.1 | 111 | 29.5 |
| Compound 53 | 112 | 750 | 79.8 |
| Compound 54B | 4.50 | 25.2 | 6.68 |
| Compound 55 | 7.32 | 56.1 | 2.67 |
| Compound 56B | 13.9 | 114 | 6.59 |
| Compound 57 | 2.25 | 10.0 | 3.22 |
| Compound 58 | 5.37 | 21.6 | 1.20 |
| Compound 59A | 19.6 | 61.8 | 13.3 |
| Compound 60 | | 84.9 | 16.5 |
| Compound 61 | 4.21 | 24.8 | 7.47 |
| Compound 62B | 2.26 | 8.23 | 2.40 |
| Compound 63 | 0.526 | 6.66 | 0.655 |
| Compound 64 | 0.902 | 12.1 | 4.97 |
| Compound 65 | 7.51 | 57.7 | 13.6 |
| Compound 66B | 0.879 | 5.91 | 2.22 |
| Compound 67 | 35.8 | 136 | 16.2 |
| Compound 68 | 12.2 | 169 | 39.3 |
| Compound 69 | 61.6 | 297 | 18.6 |
| Compound 70 | 23.3 | 353 | 192 |
| Compound 71 | 41.2 | 155 | 40.9 |
| Compound 72 | 20.9 | 145 | 79.6 |
| Compound 73 | 6.87 | 128 | 28.9 |
| Compound 74 | 112 | 126 | 37.3 |
| Compound 75 | 109 | 806 | 163 |
| Compound 76 | 5.70 | 76.0 | 7.86 |
| Compound 77 | 26.3 | 103 | — |
| Compound 79 | 22.8 | 110 | 70.7 |
| Compound 82 | 370 | 920 | 128 |
| Compound 83 | 3.46 | 9.47 | 44.5 |
| Compound 84 | 107 | 2191 | 119 |
| Compound 85 | 238 | 824 | 383 |
| Compound 86 | | | 412 |
| Compound 88 | 2.90 | 10.9 | 8.03 |
| Compound 89 | 0.894 | 2.67 | 1.98 |
| Compound 90 | 36.8 | 415 | 40.6 |
| Compound 91A | 20.8 | 105 | 59.6 |
| Compound 92A | 137 | 555 | 17.5 |
| Compound 92B | 47.2 | 285 | 22.1 |
| Compound 94 | 19.5 | 82.5 | 6.09 |
| Compound 95 | 17.8 | 1.87 | 1.00 |
| Compound 96 | | | 216 |
| Compound 97 | 112 | 338 | 82.9 |
| Compound 99 | 55.7 | 219 | 73.4 |
| Compound 100 | 50.1 | 444 | 119 |
| Compound 101 | 128 | 656 | 199 |
| Compound 102 | 121 | 274 | 27.1 |
| Compound 103 | 30.8 | 180 | 14.5 |
| Compound 106B | 12.1 | 50.8 | 8.41 |
| Compound 108A | 15.9 | 62.7 | 15.8 |
| Compound 110 | 30.7 | 324 | 106 |
| Compound 111 | 179 | 410 | 159 |
| Compound 113 | 35.1 | 587 | 45.4 |
| Compound 114 | 147 | 586 | 45.8 |
| Compound 116 | | 237 | 23.6 |
| Compound 119 | 3.75 | 52.1 | 14.7 |
| Compound 120 | 12.1 | 47.2 | 39.0 |
| Compound 121 | 17.8 | 140 | 49.8 |
| Compound 122 | 25.9 | 114 | 24.8 |
| Compound 124 | 136 | 508 | 145 |
| Compound 125 | 3.67 | 15.9 | 2.59 |
| Compound 127 | 19.8 | 173 | 62.6 |
| Compound 128 | 3.43 | 10.8 | 3.26 |
| Compound 138 | 56.0 | 143 | 43.0 |
| Compound 154 | 60.9 | 189 | 41.7 |
| Compound 155B | 7.63 | 38.4 | 16.5 |
| Compound 156A | 42.1 | 156 | 26.6 |
| Compound 156C | 12.1 | 237 | 25.4 |
| Compound 158 | 0.967 | 5.06 | 2.17 |
| Compound 159A | 71.4 | 687 | 45.7 |
| Compound 159B | 23.4 | 307 | 28.4 |
| Compound 160 | 14.2 | 1357 | 55.3 |
| Compound 161 | 18.5 | 64.4 | 25.1 |
| Compound 162 | 93.9 | 538 | 139 |
| Compound 163B | 1.19 | 14.0 | 2.15 |
| Compound 164 | 57.7 | 293 | 102 |
| Compound 166 | 4.54 | 36.8 | 25.5 |
| Compound 167 | 11.5 | 248 | 110 |
| Compound 169 | 15.3 | 93.6 | 15.2 |
| Compound 170 | 265 | 474 | 146 |
| Compound 171 | 73.9 | 127 | 43.7 |
| Compound 173 | 9.99 | 51.2 | 7.40 |
| Compound 175 | 7.39 | 33.0 | 6.27 |

### 5. Mouse Pharmacokinetic Study

The purpose of this study was to evaluate the pharmacokinetic properties of compounds in Balb/c mouse (female) following single dose administration. Six mice were needed for each compound and the six mice were divided into two groups (n=3/group), group A and group B. Mice in group A were treated with a single 3 mg/kg dose of compound (iv). Mice in group B were treated with a single 10 mg/kg dose of compound (po). Foreach mouse in group A, blood samples were collected at the time point of 0.083 h 0.5 h, 1 h, 2 h, 4 h, 8 h and 24 h post-dose. For each mouse in group B, blood samples were collected at the time point of 0.25 h, 0.5 h, 1 h, 2 h, 4 h, 8 h and 24 h post-dose. Blood samples were placed on ice until centrifugation to obtain plasma samples. The plasma samples were stored at −80° C. until analysis. The concentration of compound in plasma samples was determined using a LC-MS/MS method. The resulted were shown in the Table 6:

TABLE 6

| | 3 mg/kg, iv | | 10 mg/kg, po | | |
|---|---|---|---|---|---|
| | Cl_obs | AUC_{0-24 h} | | AUC_{0-24 h} | |
| | (mL/min/ | (ng · h/ | Cₘₐₓ | (ng · h/ | F |
| Compound | kg) | mL) | (ng/mL) | mL) | % |
| Compound 1 | 6.33 | 7991 | 1143 | 4591 | 17.2 |
| Compound 38A | 12.7 | 4004 | 971 | 5213 | 39.1 |
| Compound 66B | 5.77 | 8951 | 1175 | 3217 | 10.8 |
| Compound 163B | 1.86 | 27262 | 5307 | 23007 | 25.3 |

1. A compound of formula (I): 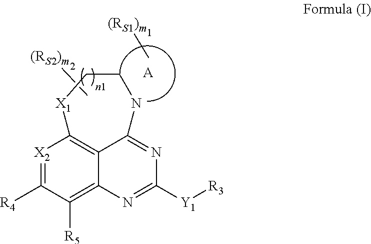
a stereoisomer thereof, a pharmaceutically acceptable salt thereof, a pharmaceutically acceptable salt of the stereoisomer, a prodrug thereof, a deuterated molecule thereof or a PROTAC molecule thereof;
wherein,
X₁ selected from the group consisting of —C(R_{X11})(R_{X12})—, —NR_{X13}—, —O—, —S—, —S(═O)— and —S(═O)₂—;
R_{X11} or R_{X12} is independently selected from the group consisting of hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, R_{X11} and R_{X12} together with the carbon atom to which they are both attached form 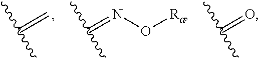
a 3-10 membered carbocyclic ring or a 3-10 heterocyclic ring; wherein, said 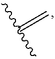
3-10 membered carbocyclic ring or 3-10 heterocyclic ring is independently unsubstituted or substituted with one or more R_{SX1};
R_{X13} is selected from the group consisting of hydrogen, deuterium, —C₁₋₆alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
X₂ is selected from the group consisting of N and CR₁;
R₁ is selected from the group consisting of hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{1A})₂, —OR_{1A}, —SR_{1A}, —S(═O)R_{1B}, —S(═O)₂R_{1B}, —C(═O)R_{1B}, —C(═O)OR_{1A}, —OC(═O)R_{1B}, —C(═O)N(R_{1A})₂, —NR_{1A}C(═O)R_{1B}, —OC(═O)OR_{1A}, —NR_{1A}C(═O)OR_{1A}, —NR_{1A}C(═S)OR_{1A}, —OC(═O)N(R_{1A})₂, —NR_{1A}C(═O)N(R_{1A})₂, —S(═O)OR_{1A}, —OS(═O)R_{1B}, —S(═O)N(R_{1A})₂, —NR_{1A}S(═O)R_{1B}, —S(═O)₂OR_{1A}, —OS(═O)₂R_{1B}, —S(═O)₂N(R_{1A})₂, —NR_{1A}S(═O)₂R_{1B}, —OS(═O)₂OR_{1A}, —NR_{1A}S(═O)₂OR_{1A}, —OS(═O)₂N(R_{1A})₂, —NR_{1A}S(═O)₂N(R_{1A})₂, —P(R_{1A})₂, —P(═O)(R_{1B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{1C})₂, —OR_{1C}, —SR_{1C}, —S(═O)R_{1D}, —S(═O)₂R_{1D}, —C(═O)R_{1D}, —C(═O)OR_{1D}, —OC(═O)R_{1D}, —C(═O)N(R_{1C})₂, —NR_{1C}C(═O)R_{1D}, —OC(═O)O_{1C}, —NR_{1C}C(═O)OR_{1C}, —NR_{1C}C(═S)OR_{1C}, —OC(═O)N(R_{1C})₂, —NR_{1C}C(═O)N(R_{1C})₂, —S(═O)OR_{1C}, —OS(═O)R_{1D}, —S(═O)N(R_{1C})₂, —NR_{1C}S(═O)R_{1D}, —S(═O)₂OR_{1C}, —OS(═O)₂R_{1D}, —S(═O)₂N(R_{1C})₂, —NR_{1C}S(═O)₂R_{1D}, —OS(═O)₂OR_{1C}, —NR_{1C}S(═O)₂OR_{1C}, —OS(═O)₂N(R_{1C})₂, —NR_{1C}S(═O)₂N(R_{1C})₂, —P(R_{1C})₂, —P(═O)(R_{1D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
n₁ is 0;
ring A is a 3-20 membered heterocyclic ring only comprising the N atom attached to the pyrimidine ring; or
ring A is a 3-20 membered heterocyclic ring comprising one or more additional heteroatoms selected from the group consisting of O, S, S═O and S(═O)₂ except the N heteroatom attached to the pyrimidine ring;
R_{S1} at each occurrence is independently selected from the group consisting of hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S1A})₂, —OR_{S1A}, —SR_{S1A}, —S(═O)R_{S1B}, —S(═O)₂R_{S1B}, —C(═O)R_{S1B}, —C(═O)OR_{S1A}, —OC(═O)R_{S1B}, —C(═O)N(R_{S1A})₂, —N_{S1A}C(═O)R_{S1B}, —OC(═O)OR_{S1A}, —N_{S1A}C(═O)OR_{S1A}, —NR_{S1A}C(═S)O_{S1A}, —OC(═O)N(R_{S1A})₂, —NR_{S1A}C(═O)N(R_{S1A})₂, —S(═O)OR_{S1A}, —OS(═O)R_{S1B}, —S(═O)N(R_{S1A})₂, —NR_{S1A}S(═O)R_{S1B}, —S(═O)₂OR_{S1A}, —OS(═O)₂R_{S1B}, —S(═O)₂N(R_{S1A})₂, —NR_{S1A}S(═O)₂R_{S1B}, —OS(═O)₂OR_{S1A}, —NR_{S1A}S(═O)₂OR_{S1A}, —OS(═O)₂N(R_{S1A})₂, —NR_{S1A}S(═O)₂N(R_{S1A})₂, —P(R_{S1A})₂, —P(═O)(R_{S1B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S1C})₂, —OR_{S1C}, —SR_{S1C}, —S(═O)R_{S1D}, —S(═O)₂R_{S1D}, —C(═O)R_{S1D}, —C(═O)OR_{S1C}, —OC(═O)R_{S1D}, —C(═O)N(R_{S1C})₂, —NR_{S1C}C(═O)R_{S1D}, —OC(═O)OR_{S1C}, —NR_{S1C}C(═O)OR_{S1C}, —NR_{S1C}C(═S)OR_{S1C}, —OC(═O)N(R_{S1C})₂, —NR_{S1C}C(═O)N(R_{S1C})₂, —S(═O)OR_{S1C}, —OS(═O)R_{S1D}, —S(═O)N(R_{S1C})₂, —NR_{S1C}S(═O)R_{S1D}, —S(═O)₂OR_{S1C}, —OS(═O)₂R_{S1D}, —S(═O)₂N(R_{S1C})₂, —NR_{S1C}S(═O)₂R_{S1D}, —OS(═O)₂OR_{S1C}, —NR_{S1C}S(═O)₂OR_{S1C}, —OS(═O)₂N(R_{S1C})₂, —NR_{S1C}S(═O)₂N(R_{S1C})₂, —P(R_{S1C})₂, —P(═O)(R_{S1D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, two R_{S1} together with the carbon atom to which they are both attached form 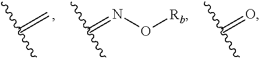
a 3-10 membered carbocyclic ring or a 3-10 heterocyclic ring; wherein, said 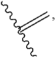
3-10 membered carbocyclic ring or 3-10 heterocyclic ring is independently unsubstituted or substituted with one or more R_{S11};
optionally, two adjacent R_{S1} together with the atoms to which they are respectively attached form 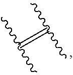 [SMILES: *=*]
a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S12};
optionally, two nonadjacent R_{S1} are connected together to form a bridge containing 0, 1, 2, 3, 4, 5 or 6 carbon atoms, wherein, each of the carbon atoms in the bridge is independently not replaced or replaced by 1 or 2 heteroatoms selected from the group consisting of O, S, S═O and S(═O)₂; the hydrogen on the each of carbon atoms or N atoms is independently unsubstituted or substituted with R_{S13};
m₁ is selected from the group consisting of 0, 1, 2, 3, 4, 5, 6, 7, 8 and 9;
R_{S2} at each occurrence is independently selected from the group consisting of hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S2A})₂, —OR_{S2A}, —SR_{S2A}, —S(═O)R_{S2B}, —S(═O)₂R_{S2B}, —C(═O)R_{S2B}, —C(═O)OR_{S2A}, —OC(═O)R_{S2B}, —C(═O)N(R_{S2A})₂, —NR_{S2A}C(═O)R_{S2B}, —OC(═O)OR_{S2A}, —NR_{S2A}C(═O)OR_{S2A}, —NR_{S2A}C(═S)OR_{S2A}, —OC(═O)N(R_{S2A})₂, —NR_{S2A}C(═O)N(R_{S2A})₂, —S(═O)OR_{S2A}, —OS(═O)R_{S2B}, —S(═O)N(R_{S2A})₂, —NR_{S2A}S(═O)R_{S2B}, —S(═O)₂OR_{S2A}, —OS(═O)₂R_{S2B}, —S(═O)₂N(R_{S2A})₂, —NR_{S2A}S(═O)₂R_{S2B}, —OS(═O)₂OR_{S2A}, —NR_{S2A}S(═O)₂OR_{S2A}, —OS(═O)₂N(R_{S2A})₂, —NR_{S2A}S(═O)₂N(R_{S2A})₂, —P(R_{S2A})₂, —P(═O)(R_{S2B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S2C})₂, —OR_{S2C}, —SR_{S2C}, —S(═O)R_{S2D}, —S(═O)₂R_{S2D}, —C(═O)R_{S2D}, —C(═O)OR_{S2D}, —OC(═O)R_{S2D}, —C(═O)N(R_{S2C})₂, —NR_{S2C}C(═O)R_{S2D}, —OC(═O)OR_{S2C}, —NR_{S2C}C(═O)OR_{S2C}, —NR_{S2C}C(═S)OR_{S2C}, —OC(═O)N(R_{S2C})₂, —NR_{S2C}C(═O)N(R_{S2C})₂, —S(═O)OR_{S2C}, —OS(═O)R_{S2D}, —S(═O)N(R_{S2C})₂, —NR_{S2C}S(═O)R_{S2D}, —S(═O)₂OR_{S2C}, —OS(═O)₂R_{S2D}, —S(═O)₂N(R_{S2C})₂, —NR_{S2C}S(═O)₂R_{S2D}, —OS(═O)₂OR_{S2C}, —NR_{S2C}S(═O)₂OR_{S2C}, —OS(═O)₂N(R_{S2C})₂, —NR_{S2C}S(═O)₂N(R_{S2C})₂, —P(R_{S2C})₂, —P(═O)(R_{S2D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, two R_{S2} together with the carbon atom to which they are both attached form 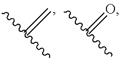
a 3-10 membered carbocyclic ring or a 3-10 heterocyclic ring; wherein, said 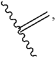
3-10 membered carbocyclic ring or 3-10 heterocyclic ring is independently unsubstituted or substituted with one or more R_{S21};
optionally, two adjacent R_{S2} together with the atoms to which they are respectively attached form 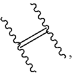 [SMILES: *=*]
a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S22};
optionally, two nonadjacent R_{S2} are connected together to form a bridge containing 0, 1, 2, 3, 4, 5 or 6 carbon atoms, wherein, each of the carbon atoms in the bridge is independently not replaced or replaced by 1 or 2 heteroatoms selected from the group consisting of N, O, S, S═O and S(═O)₂; the hydrogen on the each of carbon atoms or N atoms is independently unsubstituted or substituted with R_{S23};
m₂ is selected from the group consisting of 0, 1, 2, 3, 4 and 5;
Y₁ is selected from the group consisting of a bond, O, S, S(═O), S(═O)₂ and NR_{Y11};
R_{Y11} is selected from the group consisting of hydrogen, deuterium, —C₁₋₆alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
R₃ is selected from the group consisting of 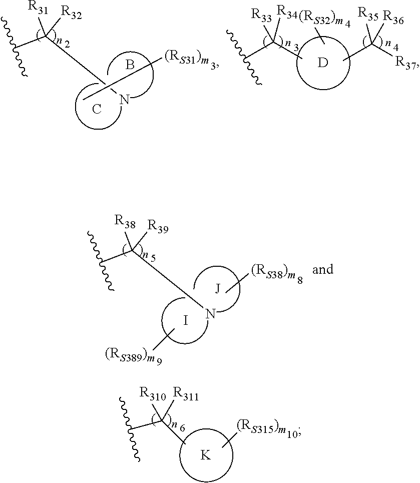
each of R₃₁, R₃₂, R₃₃, R₃₄, R₃₅, R₃₆, R₃₈, R₃₉, R₃₁₀ and R₃₁₁ is independently selected from the group consisting of hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, —S(═O)R_{B}, —S(═O)₂R_{B}, —C(═O)R_{B}, —C(═O)OR_{A}, —C(═O)N(R_{A})₂, —S(═O)OR_{A}, —S(═O)N(R_{A})₂, —S(═O)₂OR_{A}, —S(═O)₂N(R_{A})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, R₃₁ and R₃₂ together with the carbon atom to which they are both attached form 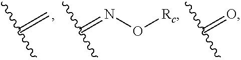
a 3-10 membered carbocyclic ring or a 3-10 heterocyclic ring; wherein, said 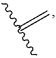
3-10 membered carbocyclic ring or 3-10 heterocyclic ring is independently unsubstituted or substituted with one or more R_{S33};
optionally, R₃₃ and R₃₄ together with the carbon atom to which they are both attached form 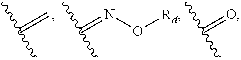
a 3-10 membered carbocyclic ring or a 3-10 heterocyclic ring; wherein, said 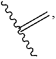
3-10 membered carbocyclic ring or 3-10 heterocyclic ring is independently unsubstituted or substituted with one or more R_{S34};
optionally, R₃₅ and R₃₆ together with the carbon atom to which they are both attached form 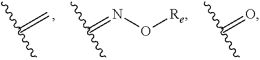
a 3-10 membered carbocyclic ring or a 3-10 heterocyclic ring; wherein, said 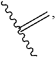
3-10 membered carbocyclic ring or 3-10 heterocyclic ring is independently unsubstituted or substituted with one or more R_{S35};
optionally, R₃₈ and R₃₉ together with the carbon atom to which they are both attached form 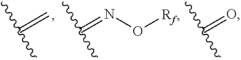
a 3-10 membered carbocyclic ring or a 3-10 heterocyclic ring; wherein, said 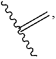
3-10 membered carbocyclic ring or 3-10 heterocyclic ring is independently unsubstituted or substituted with one or more R_{S310};
optionally, R₃₁₀ and R₃₁₁ together with the carbon atom to which they are both attached form 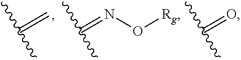
a 3-10 membered carbocyclic ring or a 3-10 heterocyclic ring; wherein, said 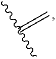
3-10 membered carbocyclic ring or 3-10 heterocyclic ring is independently unsubstituted or substituted with one or more R_{S316};
n₂ is selected from the group consisting of 0, 1, 2, 3, 4, 5 and 6;
n₃ is selected from the group consisting of 0, 1, 2, 3, 4, 5 and 6;
n₄ is selected from the group consisting of 0, 1, 2, 3, 4, 5 and 6;
n₅ is selected from the group consisting of 0, 1, 2, 3, 4, 5 and 6;
n₆ is selected from the group consisting of 0, 1, 2, 3, 4, 5 and 6;
ring B is a 3-10 membered heterocyclic ring optionally further containing 1, 2, or 3 heteroatoms selected from the group consisting of N, O, S, S(═O) and S(═O)₂;
ring C is a 3-10 membered heterocyclic ring optionally further containing 1, 2, or 3 heteroatoms selected from the group consisting of N, O, S, S(═O) and S(═O)₂;
ring D is a 3-10 membered carbocyclic ring or a 3-10 membered heterocyclic ring;
ring I is a 3-10 membered carbocyclic ring or a 3-10 membered heterocyclic ring containing 1, 2, or 3 heteroatoms selected from the group consisting of N, O, S, S(═O) and S(═O)₂;
ring J is a 3-10 membered carbocyclic ring or a 3-10 membered heterocyclic ring containing 1, 2, or 3 heteroatoms selected from the group consisting of N, O, S, S(═O) and S(═O)₂;
ring K is 3-10 membered carbocyclic ring or a 3-10 membered heterocyclic ring containing 1, 2, or 3 heteroatoms selected from the group consisting of N, O, S, S(═O) and S(═O)₂;
R_{S31} at each occurrence is independently selected from the group consisting of hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S31A})₂, —OR_{S31A}, —SR_{S31A}, —S(═O)R_{S31B}, —S(═O)₂R_{S31B}, —C(═O)R_{S31B}, —C(═O)OR_{S31A}, —OC(═O)R_{S31B}, —C(═O)N(R_{S31A})₂, —NR_{S31A}C(═O)R_{S31B}, —OC(═O)OR_{S31A}, —NR_{S31A}C(═O)OR_{S31A}, —NR_{S31A}C(═S)OR_{S31A}, —OC(═O)N(R_{S31A})₂, —NR_{S31A}C(═O)N(R_{S31A})₂, —S(═O)OR_{S31A}, —OS(═O)R_{S31B}, —S(═O)N(R_{S31A})₂, —NR_{S31A}S(═O)R_{S31B}, —S(═O)₂OR_{S31A}, —OS(═O)₂R_{S31B}, —S(═O)₂N(R_{S31A})₂, —NR_{S31A}S(═O)₂R_{S31B}, —OS(═O)₂OR_{S31A}, —NR_{S31A}S(═O)₂OR_{S31A}, —OS(═O)₂N(R_{S31A})₂, —NR_{S31A}S(═O)₂N(R_{S31A})₂, —P(R_{S31A})₂, —P(═O)(R_{S31B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S31C})₂, —OR_{S31C}, —SR_{S31C}, —S(═O)R_{S31D}, —S(═O)₂R_{S31D}, —C(═O)R_{S31D}, —C(═O)OR_{S31C}, —OC(═O)R_{S31D}, —C(═O)N(R_{S31C})₂, —NR_{S31C}C(═O)R_{S31D}, —OC(═O)OR_{S31C}, —NR_{S31C}C(═O)OR_{S31C}, —NR_{S31C}C(═S)OR_{S31C}, —OC(═O)N(R_{S31C})₂, —NR_{S31C}C(═O)N(R_{S31C})₂, —S(═O)OR_{S31C}, —OS(═O)R_{S31D}, —S(═O)N(R_{S31C})₂, —NR_{S31C}S(═O)R_{S31D}, —S(═O)₂OR_{S31C}, —OS(═O)₂R_{S31D}, —S(═O)₂N(R_{S31C})₂, —NR_{S31C}S(═O)₂R_{S31D}, —OS(═O)₂OR_{S31C}, —NR_{S31C}S(═O)₂OR_{S31C}, —OS(═O)₂N(R_{S31C})₂, —NR_{S31C}S(═O)₂N(R_{S31C})₂, —P(R_{S31C})₂, —P(═O)(R_{S31D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, two R_{S31} together with the carbon atom to which they are both attached form 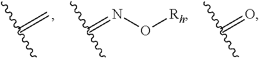
a 3-10 membered carbocyclic ring or a 3-10 membered heterocyclic ring; wherein, said 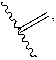
3-10 membered carbocyclic ring or 3-10 heterocyclic ring is independently unsubstituted or substituted with one or more R_{S311};
optionally, two adjacent R_{S31} together with the carbon atoms to which they are respectively attached form 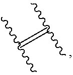 [SMILES: *=*]
a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S312};
optionally, two nonadjacent R_{S31} are connected together to form a bridge containing 0, 1, 2, 3, 4, 5 or 6 carbon atoms, wherein, each of the carbon atoms in the bridge is independently not replaced or replaced by 1 or 2 heteroatoms selected from the group consisting of N, O, S, S═O and S(═O)₂; the hydrogen on the each of carbon atoms or N atoms is independently unsubstituted or substituted with R_{S313};
m₃ is selected from the group consisting of 0, 1, 2, 3, 4, 5, 6, 7, 8, 9, 10, 11, and 12;
R_{S32} at each occurrence is independently selected from the group consisting of hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S32A})₂, —OR_{S32A}, —SR_{S32A}, —S(═O)R_{S32B}, —S(═O)₂R_{S32B}, —C(═O)R_{S32B}, —C(═O)OR_{S32A}, —OC(═O)R_{S32B}, —C(═O)N(R_{S32A})₂, —NR_{S32A}C(═O)R_{S32B}, —OC(═O)OR_{S32A}, —NR_{S32A}C(═O)OR_{S32A}, —NR_{S32A}C(═S)OR_{S32A}, —OC(═O)N(R_{S32A})₂, —NR_{S32A}C(═O)N(R_{S32A})₂, —S(═O)OR_{S32A}, —OS(═O)R_{S32B}, —S(═O)N(R_{S32A})₂, —NR_{S32A}S(═O)R_{S32B}, —S(═O)₂OR_{S32A}, —OS(═O)₂R_{S32B}, —S(═O)₂N(R_{S32A})₂, —NR_{S32A}S(═O)₂R_{S32B}, —OS(═O)₂OR_{S32A}, —NR_{S32A}S(═O)₂OR_{S32A}, —OS(═O)₂N(R_{S32A})₂, —NR_{S32A}S(═O)₂N(R_{S32A})₂, —P(R_{S32A})₂, —P(═O)(R_{S32B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S32C})₂, —OR_{S32C}, —SR_{S32C}, —S(═O)R_{S32C}, —S(═O)₂R_{S32D}, —C(═O)R_{S32D}, —C(═O)OR_{S32C}, —OC(═O)R_{S32D}, —C(═O)N(R_{S32C})₂, —NR_{S32C}C(═O)R_{S32D}, —OC(═O)OR_{S32C}, —NR_{S32C}C(═O)OR_{S32C}, —NR_{S32C}C(═S)OR_{S32C}, —OC(═O)N(R_{S32C})₂, —NR_{S32C}C(═O)N(R_{S32C})₂, —S(═O)OR_{S32C}, —OS(═O)R_{S32C}, —S(═O)N(R_{S32C})₂, —NR_{S32C}S(═O)R_{S32D}, —S(═O)₂OR_{S32C}, —OS(═O)₂R_{S32D}, —S(═O)₂N(R_{S32C})₂, —NR_{S32C}S(═O)₂R_{S32D}, —OS(═O)₂OR_{S32C}, —NR_{S32C}S(═O)₂OR_{S32C}, —OS(═O)₂N(R_{S32C})₂, —NR_{S32C}S(═O)₂N(R_{S32C})₂, —P(R_{S32C})₂, —P(═O)(R_{S32D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, two R_{S32} together with the carbon atom to which they are both attached form 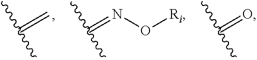
a 3-10 membered carbocyclic ring or a 3-10 heterocyclic ring; wherein, said 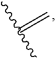
3-10 membered carbocyclic ring or 3-10 heterocyclic ring is independently unsubstituted or substituted with one or more R_{S321};
optionally, two adjacent R_{S32} together with the carbon atoms to which they are respectively attached form 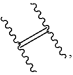 [SMILES: *=*]
a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S322};
optionally, two nonadjacent R_{S32} are connected together to form a bridge containing 0, 1, 2, 3, 4, 5 or 6 carbon atoms, wherein, each of the carbon atoms in the bridge is independently not replaced or replaced by 1 or 2 heteroatoms selected from the group consisting of N, O, S, S═O and S(═O)₂; the hydrogen on the each of carbon atoms or N atoms is independently unsubstituted or substituted with R_{S323};
m₄ is selected from the group consisting of 0, 1, 2, 3, 4, 5 and 6;
R₃₇ is selected from the group consisting of —N(R_{37A})₂ and 3-10 membered heterocyclyl, wherein said 3-10 membered heterocyclyl is optionally independently substituted with one or more R_{S37};
R_{S38} at each occurrence is independently selected from the group consisting of hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S38A})₂, —OR_{S38A}, —SR_{S38A}, —S(═O)R_{S38B}, —S(═O)₂R_{S38B}, —C(═O)R_{S38B}, —C(═O)OR_{S38A}, —OC(═O)R_{S38B}, —C(═O)N(R_{S38A})₂, —NR_{S38A}C(═O)R_{S38B}, —OC(═O)OR_{S38A}, —NR_{S38A}C(═O)OR_{S38A}, —NR_{S38A}C(═S)OR_{S38A}, —OC(═O)N(R_{S38A})₂, —NR_{S38A}C(═O)N(R_{S38A})₂, —S(═O)OR_{S38A}, —OS(═O)R_{S38B}, —S(═O)N(R_{S38A})₂, —NR_{S38A}S(═O)R_{S38B}, —S(═O)₂OR_{S38A}, —OS(═O)₂R_{S38B}, —S(═O)₂N(R_{S38A})₂, —NR_{S38A}S(═O)₂R_{S38B}, —OS(═O)₂OR_{S38A}, —NR_{S38A}S(═O)₂OR_{S38A}, —OS(═O)₂N(R_{S38A})₂, —NR_{S38A}S(═O)₂N(R_{S38A})₂, —P(R_{S38A})₂, —P(═O)(R_{S38B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S38C})₂, —OR_{S38C}, —SR_{S38C}, —S(═O)R_{S38D}, —S(═O)₂R_{S38D}, —C(═O)R_{S38D}, —C(═O)OR_{S38C}, —OC(═O)R_{S38D}, —C(═O)N(R_{S38C})₂, —NR_{S38C}C(═O)R_{S38D}, —OC(═O)OR_{S38C}, —NR_{S38C}C(═O)OR_{S38C}, —NR_{S38C}C(═S)OR_{S38C}, —OC(═O)N(R_{S38C})₂, —NR_{S38C}C(═O)N(R_{S38C})₂, —S(═O)OR_{S38C}, —OS(═O)R_{S38D}, —S(═O)N(R_{S38C})₂, —NR_{S38C}S(═O)R_{S38D}, —S(═O)₂OR_{S38C}, —OS(═O)₂R_{S38D}, —S(═O)₂N(R_{S38C})₂, —NR_{S38C}S(═O)₂R_{S38D}, —OS(═O)₂OR_{S38C}, —NR_{S38C}S(═O)₂OR_{S38C}, —OS(═O)₂N(R_{S38C})₂, —NR_{S38C}S(═O)₂N(R_{S38C})₂, —P(R_{S38C})₂, —P(═O)(R_{S38D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, two R_{S38} together with the carbon atom to which they are both attached form 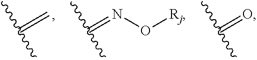
a 3-10 membered carbocyclic ring or a 3-10 membered heterocyclic ring; wherein, said 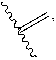
3-10 membered carbocyclic ring or 3-10 heterocyclic ring is independently unsubstituted or substituted with one or more R_{S381};
optionally, two adjacent R_{S38} together with the carbon atoms to which they are respectively attached form 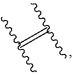 [SMILES: *=*]
a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S382};
optionally, two nonadjacent R_{S38} are connected together to form a bridge containing 0, 1, 2, 3, 4, 5 or 6 carbon atoms, wherein, each of the carbon atoms in the bridge is independently not replaced or replaced by 1 or 2 heteroatoms selected from the group consisting of N, O, S, S═O and S(═O)₂; the hydrogen on the each of carbon atoms or N atoms is independently unsubstituted or substituted with R_{S383};
m₈ is selected from the group consisting of 0, 1, 2, 3, 4, 5, 6, 7, 8, 9, 10, 11, and 12;
R_{S39} at each occurrence is independently selected from the group consisting of hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S39A})₂, —OR_{S39A}, —SR_{S39A}, —S(═O)R_{S39B}, —S(═O)₂R_{S39B}, —C(═O)R_{S39B}, —C(═O)OR_{S39A}, —OC(═O)R_{S39B}, —C(═O)N(R_{S39A})₂, —NR_{S39A}C(═O)R_{S39B}, —OC(═O)OR_{S39A}, —NR_{S39A}C(═O)OR_{S39A}, —NR_{S39A}C(═S)OR_{S39A}, —OC(═O)N(R_{S39A})₂, —NR_{S39A}C(═O)N(R_{S39A})₂, —S(═O)OR_{S39A}, —OS(═O)R_{S39B}, —S(═O)N(R_{S39A})₂, —NR_{S39A}S(═O)R_{S39B}, —S(═O)₂OR_{S39A}, —OS(═O)₂R_{S39B}, —S(═O)₂N(R_{S39A})₂, —NR_{S39A}S(═O)₂R_{S39B}, —OS(═O)₂OR_{S39A}, —NR_{S39A}S(═O)₂OR_{S39A}, —OS(═O)₂N(R_{S39A})₂, —NR_{S39A}S(═O)₂N(R_{S39A})₂, —P(R_{S39A})₂, —P(═O)(R_{S39B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S39C})₂, —OR_{S39C}, —SR_{S39C}, —S(═O)R_{S39C}, —S(═O)₂R_{S39D}, —C(═O)R_{S39D}, —C(═O)OR_{S39C}, —OC(═O)R_{S39D}, —C(═O)N(R_{S39C})₂, —NR_{S39C}C(═O)R_{S39D}, —OC(═O)OR_{S39C}, —NR_{S39C}C(═O)OR_{S39C}, —NR_{S39C}C(═S)OR_{S39C}, —OC(═O)N(R_{S39C})₂, —NR_{S39C}C(═O)N(R_{S39C})₂, —S(═O)OR_{S39C}, —OS(═O)R_{S39C}, —S(═O)N(R_{S39C})₂, —NR_{S39C}S(═O)R_{S39D}, —S(═O)₂OR_{S39C}, —OS(═O)₂R_{S39D}, —S(═O)₂N(R_{S39C})₂, —NR_{S39C}S(═O)₂R_{S39D}, —OS(═O)₂OR_{S39C}, —NR_{S39C}S(═O)₂OR_{S39C}, —OS(═O)₂N(R_{S39C})₂, —NR_{S39C}S(═O)₂N(R_{S39C})₂, —P(R_{S39C})₂, —P(═O)(R_{S39D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, two R_{S39} together with the carbon atom to which they are both attached form 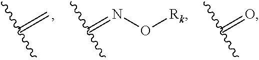
a 3-10 membered carbocyclic ring or a 3-10 heterocyclic ring; wherein, said 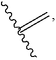
3-10 membered carbocyclic ring or 3-10 heterocyclic ring is independently unsubstituted or substituted with one or more R_{S391};
optionally, two adjacent R_{S39} together with the carbon atoms to which they are respectively attached form 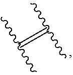 [SMILES: *=*]
a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S392};
optionally, two nonadjacent R_{S39} are connected together to form a bridge containing 0, 1, 2, 3, 4, 5 or 6 carbon atoms, wherein, each of the carbon atoms in the bridge is independently not replaced or replaced by 1 or 2 heteroatoms selected from the group consisting of N, O, S, S═O and S(═O)₂; the hydrogen on the each of carbon atoms or N atoms is independently unsubstituted or substituted with R_{S393};
m₉ is selected from the group consisting of 0, 1, 2, 3, 4, 5 and 6;
R_{S315} at each occurrence is independently selected from the group consisting of hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S315A})₂, —OR_{S315A}, —SR_{S315A}, —S(═O)R_{S315B}, —S(═O)₂R_{S315B}, —C(═O)R_{S315B}, —C(═O)OR_{S315A}, —OC(═O)R_{S315B}, —C(═O)N(R_{S315A})₂, —NR_{S315A}C(═O)R_{S315B}, —OC(═O)OR_{S315A}, —NR_{S315A}C(═O)OR_{S315A}, —NR_{S315A}C(═S)OR_{S315A}, —OC(═O)N(R_{S315A})₂, —NR_{S315A}C(═O)N(R_{S315A})₂, —S(═O)OR_{S315A}, —OS(═O)R_{S315B}, —S(═O)N(R_{S315A})₂, —NR_{S315A}S(═O)R_{S315B}, —S(═O)₂OR_{S315A}, —OS(═O)₂R_{S315B}, —S(═O)₂N(R_{S315A})₂, —NR_{S315A}S(═O)₂R_{S315B}, —OS(═O)₂OR_{S315A}, —NR_{S315A}S(═O)₂OR_{S315A}, —OS(═O)₂N(R_{S315A})₂, —NR_{S315A}S(═O)₂N(R_{S315A})₂, —P(R_{S315A})₂, —P(═O)(R_{S315B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S315C})₂, —OR_{S315C}, —SR_{S315C}, —S(═O)R_{S315C}, —S(═O)₂R_{S315D}, —C(═O)R_{S315D}, —C(═O)OR_{S315C}, —OC(═O)R_{S315D}, —C(═O)N(R_{S315C})₂, —NR_{S315C}C(═O)R_{S315D}, —OC(═O)OR_{S315C}, —NR_{S315C}C(═O)OR_{S315C}, —NR_{S315C}C(═S)OR_{S315C}, —OC(═O)N(R_{S315C})₂, —NR_{S315C}C(═O)N(R_{S315C})₂, —S(═O)OR_{S315C}, —OS(═O)R_{S315C}, —S(═O)N(R_{S315C})₂, —NR_{S315C}S(═O)R_{S315D}, —S(═O)₂OR_{S315C}, —OS(═O)₂R_{S315D}, —S(═O)₂N(R_{S315C})₂, —NR_{S315C}S(═O)₂R_{S315D}, —OS(═O)₂OR_{S315C}, —NR_{S315C}S(═O)₂OR_{S315C}, —OS(═O)₂N(R_{S315C})₂, —NR_{S315C}S(═O)₂N(R_{S315C})₂, —P(R_{S315C})₂, —P(═O)(R_{S315D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, two R_{S315} together with the carbon atom to which they are both attached form 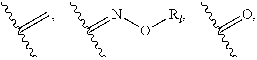
a 3-10 membered carbocyclic ring or a 3-10 heterocyclic ring; wherein, said 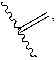
3-10 membered carbocyclic ring or 3-10 heterocyclic ring is independently unsubstituted or substituted with one or more R_{S3151};
optionally, two adjacent R_{S315} together with the carbon atoms to which they are respectively attached form 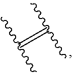 [SMILES: *=*]
a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S3152};
optionally, two nonadjacent R_{S315} are connected together to form a bridge containing 0, 1, 2, 3, 4, 5 or 6 carbon atoms, wherein, each of the carbon atoms in the bridge is independently not replaced or replaced by 1 or 2 heteroatoms selected from the group consisting of N, O, S, S═O and S(═O)₂; the hydrogen on the each of carbon atoms or N atoms is independently unsubstituted or substituted with R_{S3153};
m₁₀ is selected from the group consisting of 0, 1, 2, 3, 4, 5 and 6;
R₄ is selected from the group consisting of 6-10 membered aryl, 5-10 membered heteroaryl, 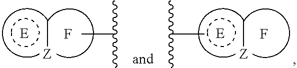
wherein said 6-10 membered aryl, 5-10 membered heteroaryl, 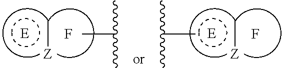
is independently unsubstituted or substituted with one or more R_{S4};
Z at each occurrence is independently selected from the group consisting of C and N;
ring E at each occurrence is independently selected from the group consisting of a 6 membered aryl ring and a 5-6 membered heteroaryl ring and ring F at each occurrence is independently selected from the group consisting of a 3-10 membered carbocyclic ring and a 3-10 membered heterocyclic ring when Z is C;
ring E at each occurrence is independently a 5-6 membered heteroaryl ring and ring F at each occurrence is independently a 3-10 membered heterocyclic ring when Z is N;
R_{S4} at each occurrence is independently selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S4A})₂, —OR_{S4A}, —SR_{S4A}, —S(═O)R_{S4B}, —S(═O)₂R_{S4B}, —C(═O)R_{S4B}, —C(═O)OR_{S4A}, —OC(═O)R_{S4B}, —C(═O)N(R_{S4A})₂, —NR_{S4A}C(═O)R_{S4B}, —OC(═O)OR_{S4A}, —NR_{S4A}C(═O)OR_{S4A}, —NR_{S4A}C(═S)OR_{S4A}, —OC(═O)N(R_{S4A})₂, —NR_{S4A}C(═O)N(R_{S4A})₂, —S(═O)OR_{S4A}, —OS(═O)R_{S4B}, —S(═O)N(R_{S4A})₂, —NR_{S4A}S(═O)R_{S4B}, —S(═O)₂OR_{S4A}, —OS(═O)₂R_{S4B}, —S(═O)₂N(R_{S4A})₂, —NR_{S4A}S(═O)₂R_{S4B}, —OS(═O)₂OR_{S4A}, —NR_{S4A}S(═O)₂OR_{S4A}, —OS(═O)₂N(R_{S4A})₂, —NR_{S4A}S(═O)₂N(R_{S4A})₂, —P(R_{S4A})₂, —P(═O)(R_{S4B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S4C})₂, —OR_{S4C}, —SR_{S4C}, —S(═O)R_{S4D}, —S(═O)₂R_{S4D}, —C(═O)R_{S4D}, —C(═O)OR_{S4D}, —OC(═O)R_{S4D}, —C(═O)N(R_{S4C})₂, —NR_{S4C}C(═O)R_{S4D}, —OC(═O)OR_{S4C}, —NR_{S4C}C(═O)OR_{S4C}, —NR_{S4C}C(═S)OR_{S4C}, —OC(═O)N(R_{S4C})₂, —NR_{S4C}C(═O)N(R_{S4C})₂, —S(═O)OR_{S4C}, —OS(═O)R_{S4D}, —S(═O)N(R_{S4C})₂, —NR_{S4C}S(═O)R_{S4D}, —S(═O)₂OR_{S4C}, —OS(═O)₂R_{S4D}, —S(═O)₂N(R_{S4C})₂, —NR_{S4C}S(═O)₂R_{S4D}, —OS(═O)₂OR_{S4C}, —NR_{S4C}S(═O)₂OR_{S4C}, —OS(═O)₂N(R_{S4C})₂, —NR_{S4C}S(═O)₂N(R_{S4C})₂, —P(R_{S4C})₂, —P(═O)(R_{S4D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
R₅ is selected from the group consisting of hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{5A})₂, —OR_{5A}, —SR_{5A}, —S(═O)R_{5B}, —S(═O)₂R_{5B}, —C(═O)R_{5B}, —C(═O)OR_{5A}, —OC(═O)R_{5B}, —C(═O)N(R_{5A})₂, —NR_{5A}C(═O)R_{5B}, —OC(═O)OR_{5A}, —NR_{5A}C(═O)OR_{5A}, —NR_{5A}C(═S)OR₅A, —OC(═O)N(R_{5A})₂, —NR_{5A}C(═O)N(R_{5A})₂, —S(═O)OR_{5A}, —OS(═O)R_{5B}, —S(═O)N(R_{5A})₂, —NR_{5A}S(═O)R_{5B}, —S(═O)₂OR_{5A}, —OS(═O)₂R_{5B}, —S(═O)₂N(R_{5A})₂, —NR_{5A}S(═O)₂R_{5B}, —OS(═O)₂OR_{5A}, —NR_{5A}S(═O)₂OR_{5A}, —OS(═O)₂N(R_{5A})₂, —NR_{5A}S(═O)₂N(R_{5A})₂, —P(R_{5A})₂, —P(═O)(R_{5B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(RSC)₂, —OR_{5C}, —SR_{5C}, —S(═O)R_{5D}, —S(═O)₂R_{5D}, —C(═O)R_{5D}, —C(═O)OR_{5D}, —OC(═O)R_{5D}, —C(═O)N(R_{5C})₂, —NR_{5C}C(═O)R_{5D}, —OC(═O)OR_{5C}, —NR_{5C}C(═O)OR_{5C}, —NR_{5C}C(═S)OR_{5C}, —OC(═O)N(R_{5C})₂, —NR_{5C}C(═O)N(R_{5C})₂, —S(═O)OR_{5C}, —OS(═O)R_{5D}, —S(═O)N(R_{5C})₂, —NR_{5C}S(═O)R_{5D}, —S(═O)₂OR_{5C}, —OS(═O)₂R_{5D}, —S(═O)₂N(R_{5C})₂, —NR_{5C}S(═O)₂R_{5D}, —OS(═O)₂OR_{5C}, —NR_{5C}S(═O)₂OR_{5C}, —OS(═O)₂N(R_{5C})₂, —NR_{5C}S(═O)₂N(R_{5C})₂, —P(R_{5C})₂, —P(═O)(R_{5D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
each of R_{1A}, R_{1C}, R_{S1A}, R_{S1C}, R_{S2A}, R_{S2C}, R_{S31A}, R_{S31C}, R_{S32A}, R_{S32C}, R_{37A}, R_{S38A}, R_{S38C}, R_{S39A}, R_{S39C}, R_{S315A}, R_{S315C}, R_{S4A}, R_{S4C}, R_{5A}, R_{5C}, Rₐ, R_{b}, R_{c}, R_{d}, Rₑ, R_{f}, R_{g}, Rₕ, Rᵢ, Rⱼ, Rₖ and Rₗ is independently selected from the group consisting of hydrogen, deuterium, —C₁₋₆alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, (two R_{1A}, two R_{1C}, two R_{S1A}, two R_{S1C}, two R_{S2A}, two R_{S2C}, two R_{S31A}, two R_{S31C}, two R_{S32A}, two R_{S32C}, two R_{37A}, two R_{S38A}, two R_{S38C}, two R_{S39A}, two R_{S39C}, two R_{S315A}, two R_{S315C}, two R_{S4A}, two R_{S4C}, two R_{5A} or two R_{5C}) together with the nitrogen atom to which they are both attached form a 3-10 membered heterocyclic ring or a 5-10 membered heteroaryl ring, wherein, said 3-10 membered heterocyclic ring or 5-10 membered heteroaryl ring is independently unsubstituted or substituted with one or more R_{SS};
each of RIB, R_{1D}, R_{S1B}, R_{S1D}, R_{S2B}, R_{S2D}, R_{S31B}, R_{S31D}, R_{S32B}, R_{S32D}, R_{S38B}, R_{S38D}, R_{S39B}, R_{S315B}, R_{S315D}, R_{S39D}, R_{S4B}, R_{S4D}, R_{5B} and R_{5D} is independently selected from the group consisting of hydrogen, deuterium, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NRCS(═O)₂R_{D}, —OS(═O)₂OR_{C}, —NR_{C}S(═O)₂OR_{C}, —OS(═O)₂NR_{C}, —NRCS(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
each of (R_{A}, R_{B}, R_{C} and R_{D}) is independently selected from the group consisting of hydrogen, deuterium, —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more R_{SA};
Each of R_{SX1}, R_{S11}, R_{S12}, R_{S13}, R_{S21}, R_{S22}, R_{S23}, R_{S33}, R_{S34}, R_{S35}, R_{S37}, R_{S310}, R_{S316}, R_{S311}, R_{S312}, R_{S313}, R_{S321}, R_{S322}, R_{S323}, R_{S381}, R_{S382}, R_{S383}, R_{S391}, R_{S392}, R_{S393}, R_{S3151}, R_{S3152}, R_{S3153}, R_{SS} and R_{SA} is independently selected from the group consisting of deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —O(C₁₋₆alkyl), —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆ alkyl)S(═O)₂(C₁₋₆alkyl), —OS(═O)₂O(C₁₋₆alkyl), —NHS(═O)₂N(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein, said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₃alkyl, haloC₁₋₃alkyl, haloC₁₋₃alkoxy, —C₂₋₃alkenyl, —C₂₋₃alkynyl, —CN, —NO₂, —N₃, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —OC(═O)O(C₁₋₃alkyl), —NHC(═O)(OC₁₋₃alkyl), —N(C₁₋₃alkyl)C(═O)(OC₁₋₃alkyl), —OC(═O)NH(C₁₋₃alkyl), —OC(═O)N(C₁₋₃alkyl)₂, —NHC(═O)NH₂, —NHC(═O)NH(C₁₋₃alkyl), —NHC(═O)N(C₁₋₃alkyl)₂, —N(C₁₋₃alkyl)C(═O)NH₂, —N(C₁₋₃alkyl)C(═O)NH(C₁₋₃alkyl), —N(C₁₋₃alkyl)C(═O)N(C₁₋₃alkyl)₂, —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —OS(═O)₂(C₁₋₃alkyl), —NHS(═O)₂O(C₁₋₃alkyl), —N(C₁₋₃alkyl)S(═O)₂O(C₁₋₃alkyl), —OS(═O)₂NH₂, —OS(═O)₂NH(C₁₋₃alkyl), —OS(═O)₂N(C₁₋₃alkyl)₂, —NHS(═O)₂NH₂, —NHS(═O)₂NH(C₁₋₃alkyl), —NHS(═O)₂N(C₁₋₃alkyl)₂, —N(C₁₋₃alkyl)S(═O)₂NH₂, —N(C₁₋₃alkyl)S(═O)₂NH(C₁₋₃alkyl), —N(C₁₋₃alkyl)S(═O)₂N(C₁₋₃alkyl)₂, —PH(C₁₋₃alkyl), —P(C₁₋₃alkyl)₂, —P(═O)H(C₁₋₃alkyl), —P(═O)(C₁₋₃alkyl)₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
each of heterocyclyl or heterocyclic at each occurrence independently contains 1, 2, 3 or 4 heteroatoms selected from the group consisting of N, O, S, S(═O) and S(═O)₂;
each of heteroaryl at each occurrence independently contains 1, 2, 3 or 4 heteroatoms selected from the group consisting of N, O, and S.

2. (canceled)

3. (canceled)

4. (canceled)

5. The compound of claim 1, wherein:
R_{X11} or R_{X12} is independently selected from the group consisting of hydrogen, deuterium, —F, methyl, —CD₃, ethyl, propyl, isopropyl and cyclopropyl;
optionally, R_{X11} and R_{X12} together with the carbon atom to which they are both attached form 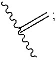
R_{X13} is independently selected from the group consisting of hydrogen, deuterium, methyl, —CD₃, ethyl, propyl, isopropyl and cyclopropyl.

6. (canceled)

7. (canceled)

8. The compound of claim 1, wherein, m₁ is selected from the group consisting of 0, 1, 2, 3, 4, 5, and 6.

9. The compound of claim 1, wherein, R_{S1} at each occurrence is independently selected from the group consisting of deuterium, halogen, —C₁₋₆alkyl, —C₂₋₆alkenyl, —C₂₋₆alkynyl, haloC₁₋₆alkyl, haloC₁₋₆alkoxy, —CN, —NH₂, —NH(C₁₋₆alkyl), —N(C₁₋₆alkyl)₂, —OH, —OC₁₋₆alkyl and 3-6 membered cycloalkyl; wherein said —C₁₋₆alkyl, —C₂₋₆alkenyl, —C₂₋₆alkynyl or 3-6 membered cycloalkyl is independently unsubstituted or substituted with 1, 2, or 3 substituents selected from the group consisting of deuterium, halogen, haloC₁₋₆alkyl, haloC₁₋₆alkoxy, —CN, —NH₂, —NH(C₁₋₆alkyl), —N(C₁₋₆alkyl)₂, —OH and —OC₁₋₆alkyl;
optionally, two R_{S1} together with the carbon atom to which they are both attached form 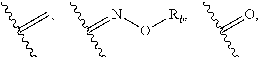
or a 3-6 membered carbocyclic ring; wherein, said 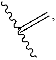
3-6 membered carbocyclic ring is independently unsubstituted or substituted with 1, 2, or 3 substituents selected from the group consisting of deuterium, halogen, haloC₁₋₆alkyl, haloC₁₋₆alkoxy, —CN, —NH₂, —NH(C₁₋₆alkyl), —N(C₁₋₆alkyl)₂, —OH and —OC₁₋₆alkyl; R_{b} is —C₁₋₆alkyl;
optionally, two adjacent R_{S1} together with the atoms to which they are respectively attached form 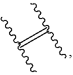 [SMILES: *=*]
a 3-6 membered carbocyclic ring; wherein, said 3-6 membered carbocyclic ring is independently unsubstituted or substituted with 1, 2, or 3 substituents selected from the group consisting of deuterium, halogen, haloC₁₋₆alkyl, haloC₁₋₆alkoxy, —CN, —NH₂, —NH(C₁₋₆alkyl), —N(C₁₋₆alkyl)₂, —OH and —OC₁₋₆alkyl.

10. The compound of claim 9, wherein, R_{S1} at each occurrence is independently selected from the group consisting of —F, —OH, —OCH₃, —CN, —CH₂F, —CF₃, —CH₂OCH₃, —CH₂CN, —CHF₂, —CD₃, —NH₂ and —CH₃; or two R_{S1} together with the carbon atom to which they are both attached form 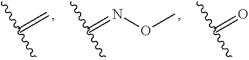
or a cyclopropyl ring; or two adjacent R_{S1} together with the atoms to which they are respectively attached form a cyclopropyl ring.

11. The compound of claim 1, wherein, the compound is selected from the group consisting of any one of the following formulas:

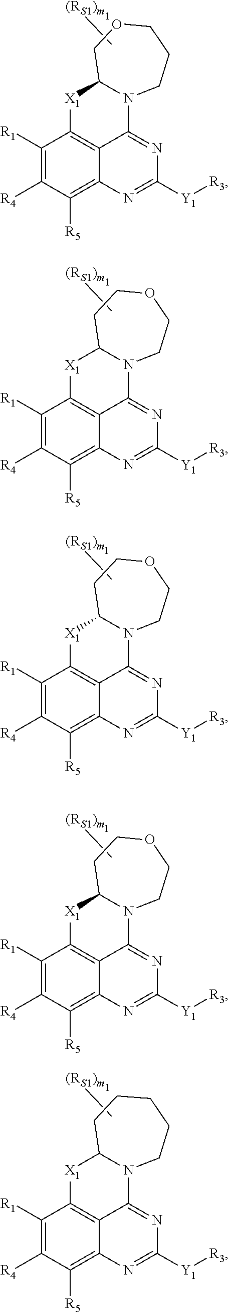

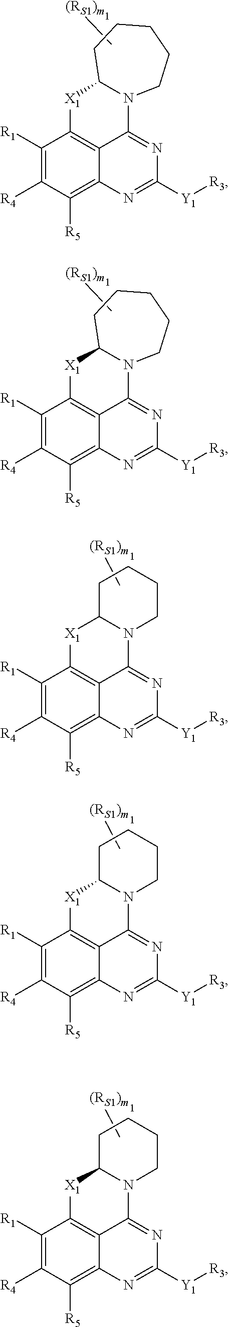

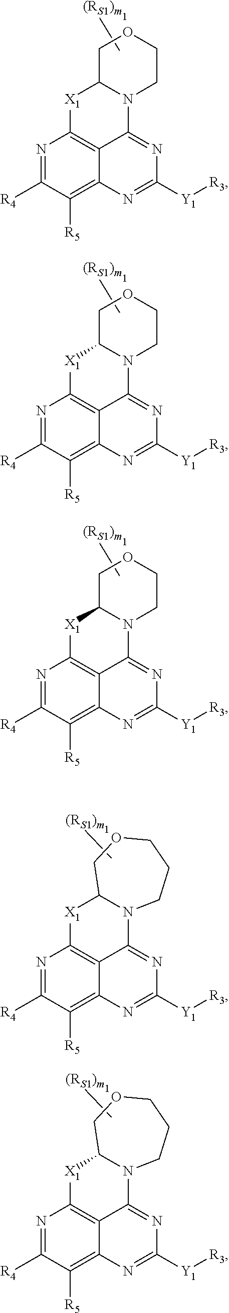

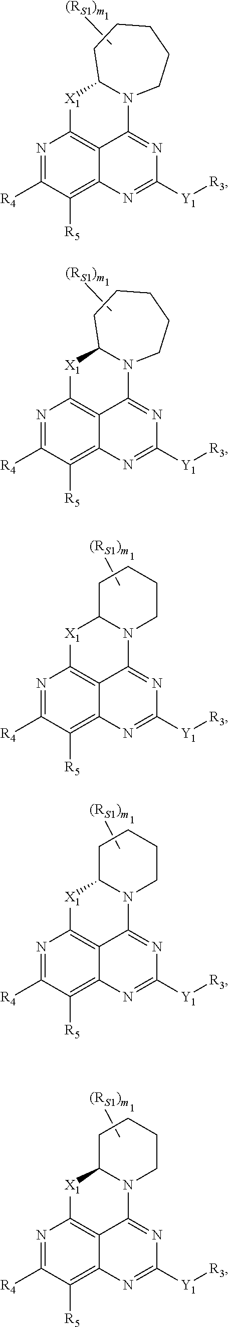

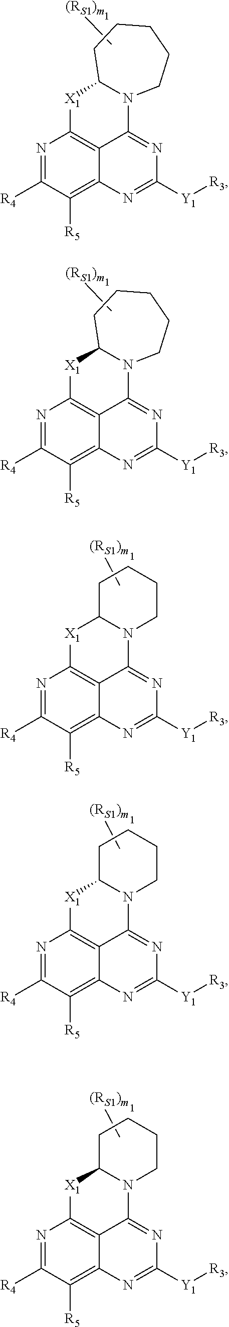

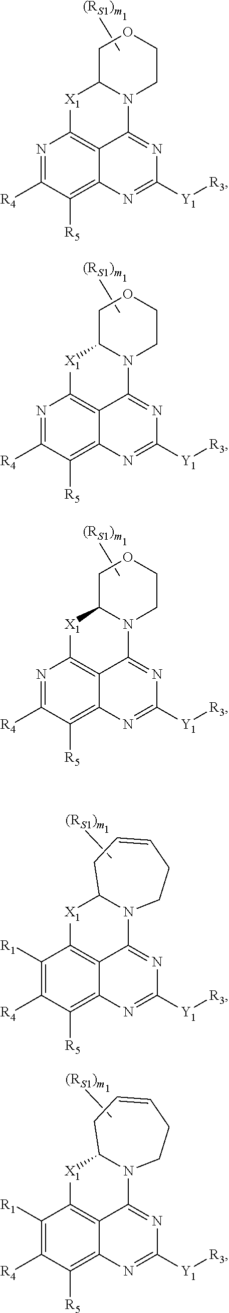

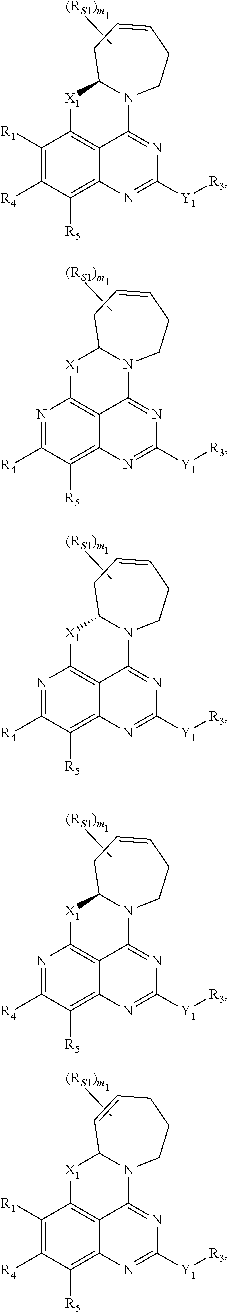

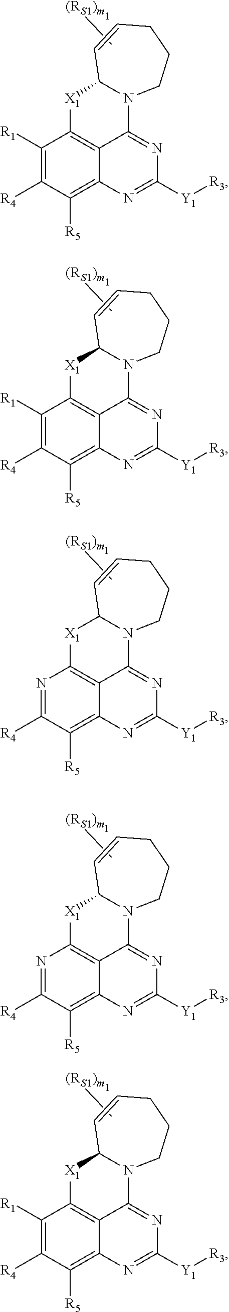

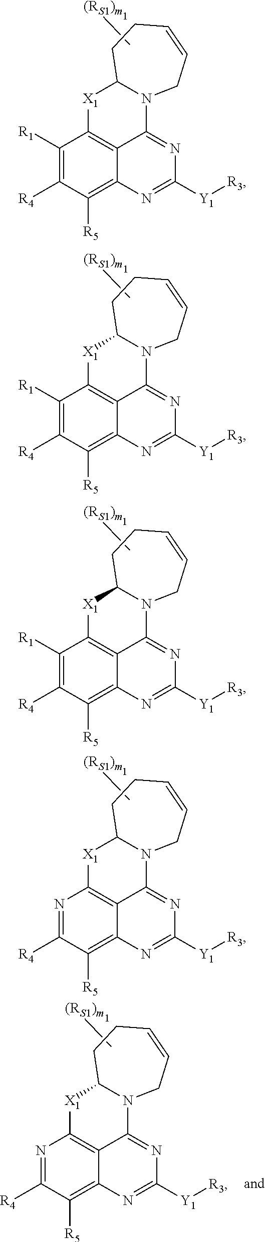

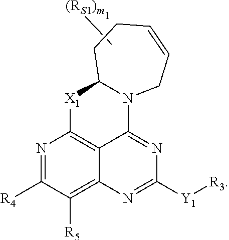

12. The compound of claim 1, wherein, the compound is selected from the group consisting of any one of the following formulas:

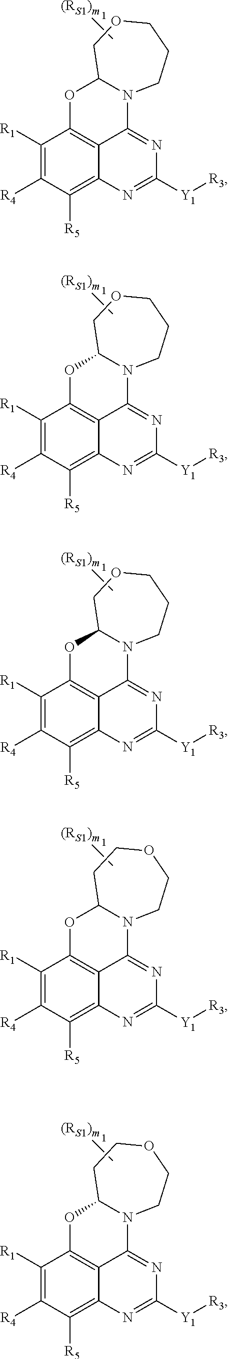

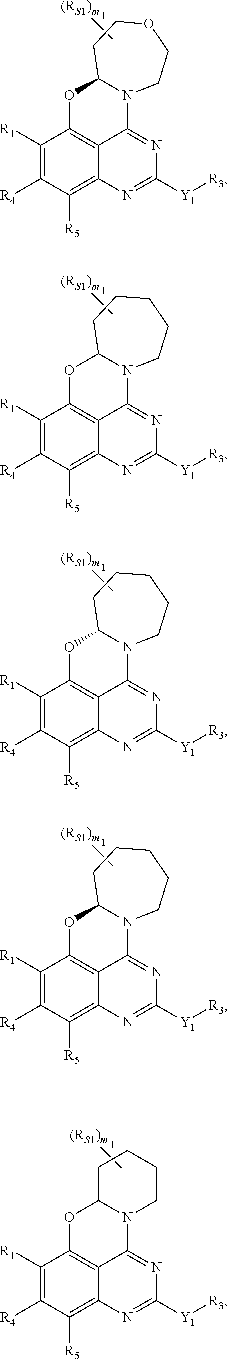

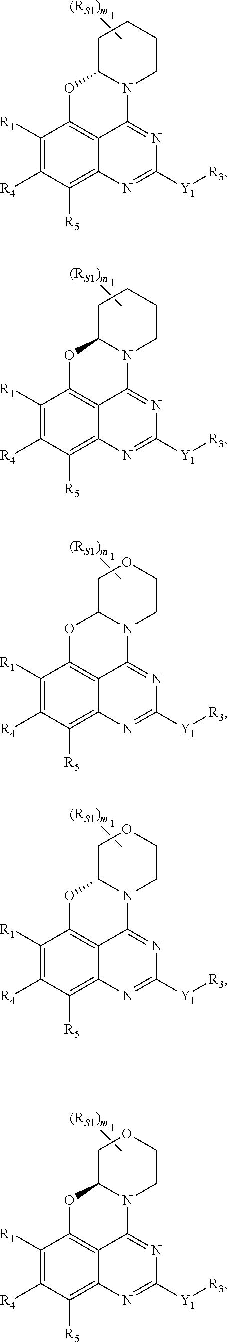

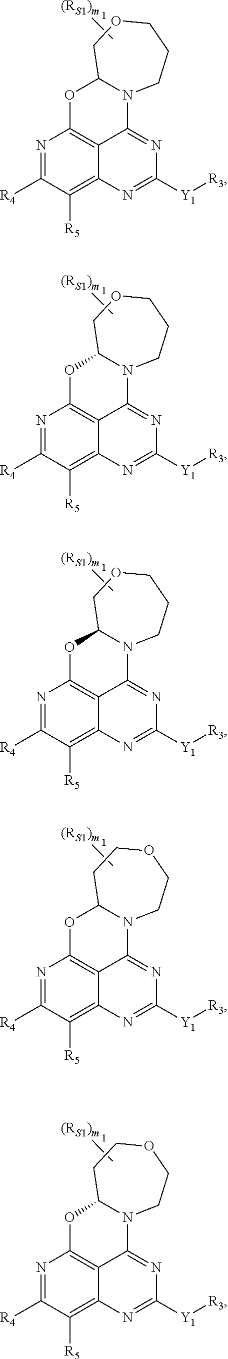

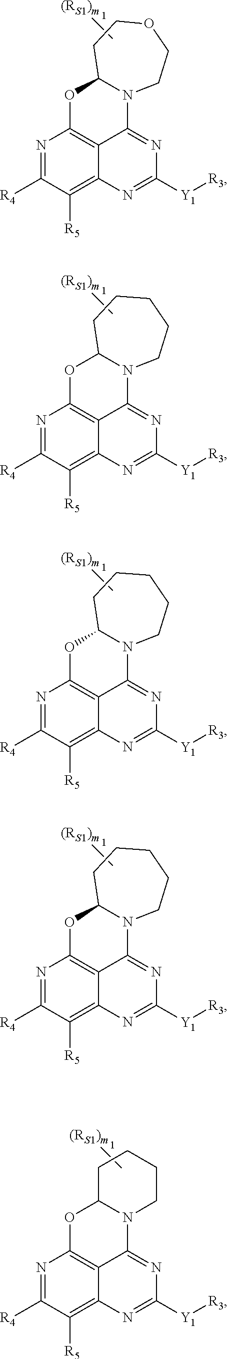

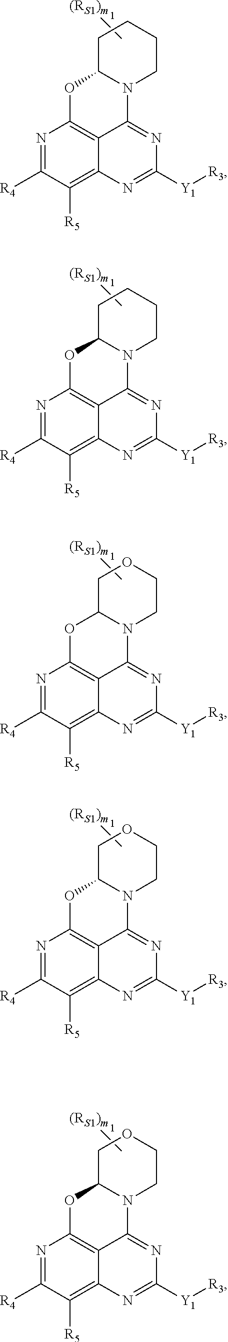

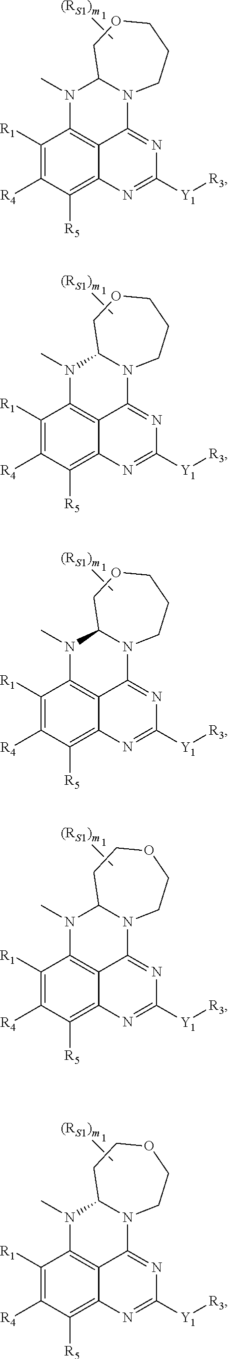

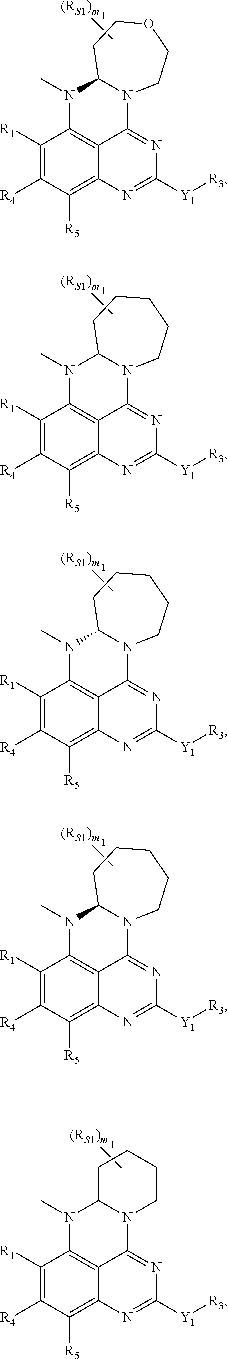

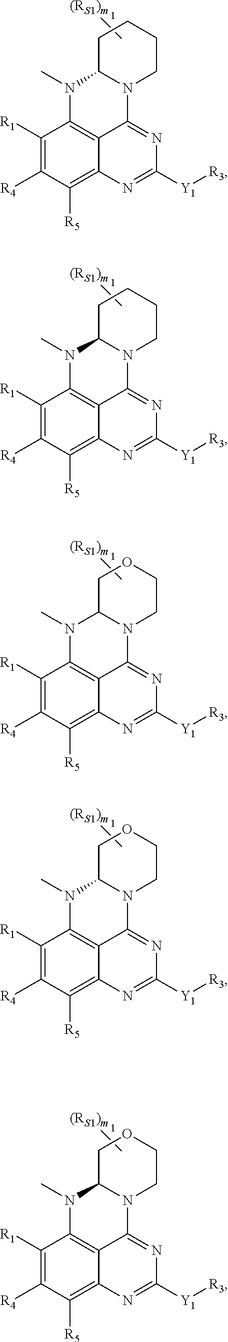

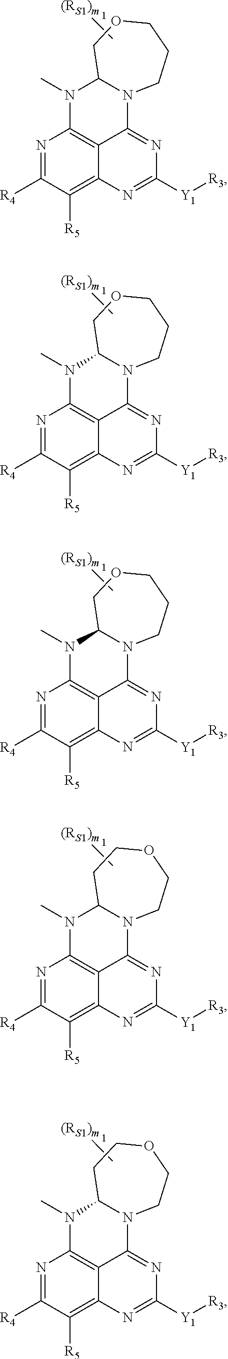

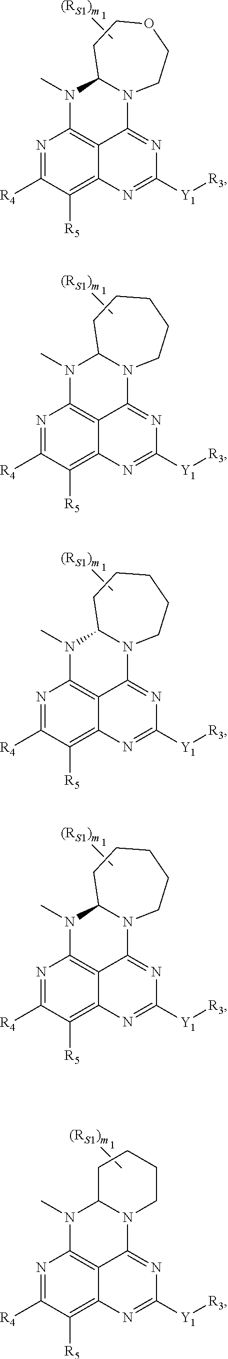

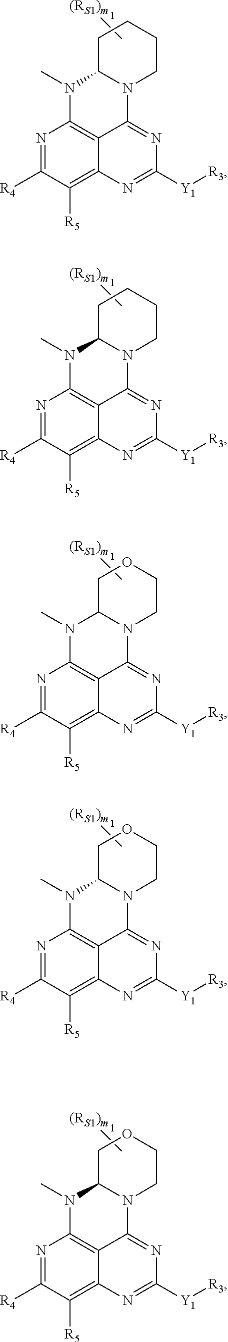

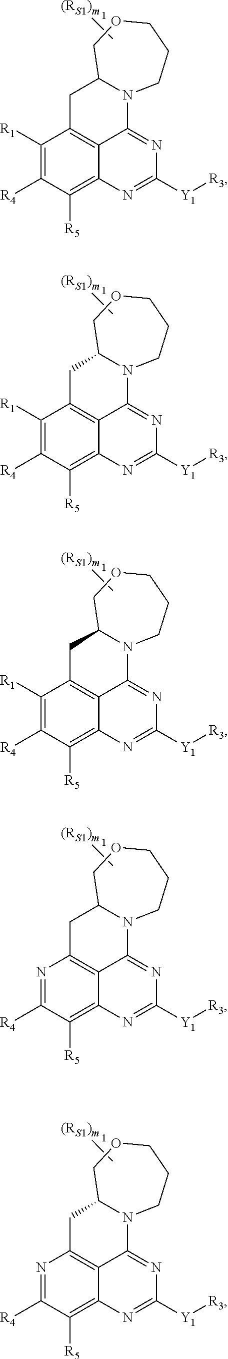

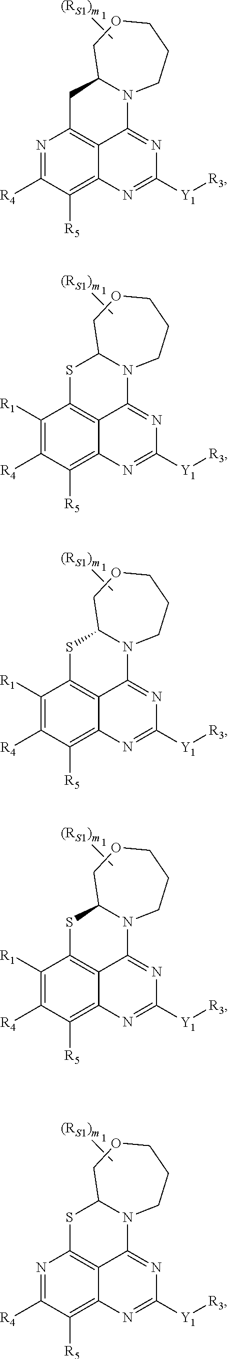

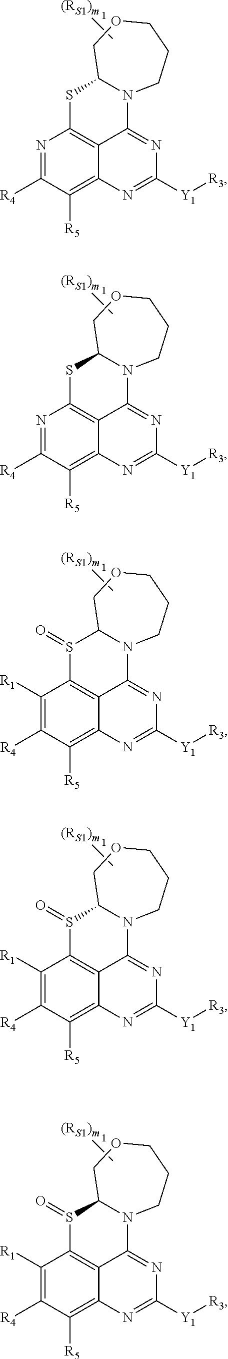

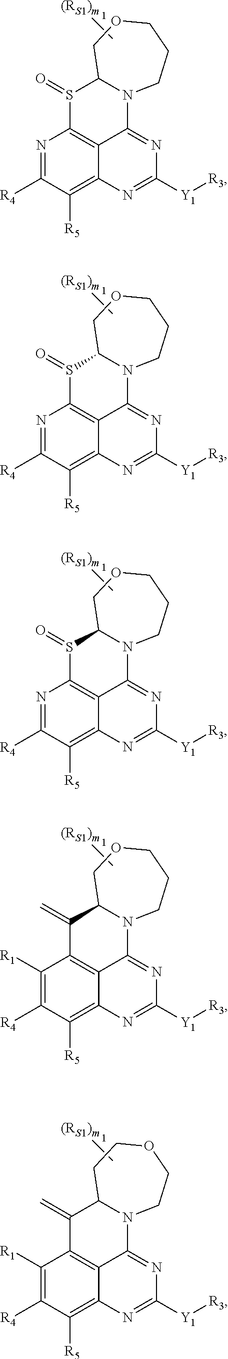

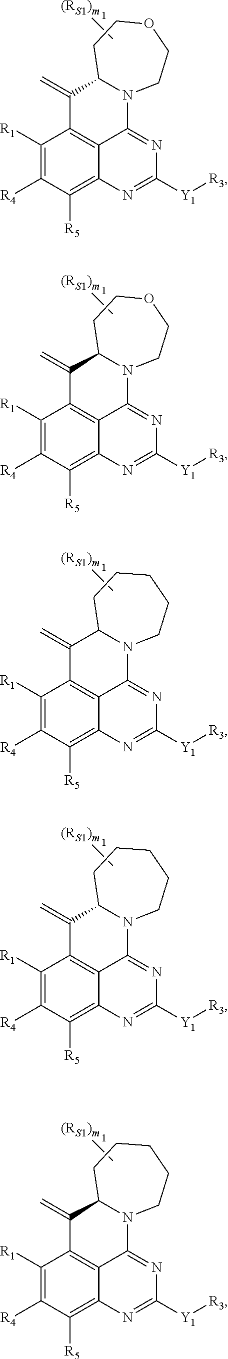

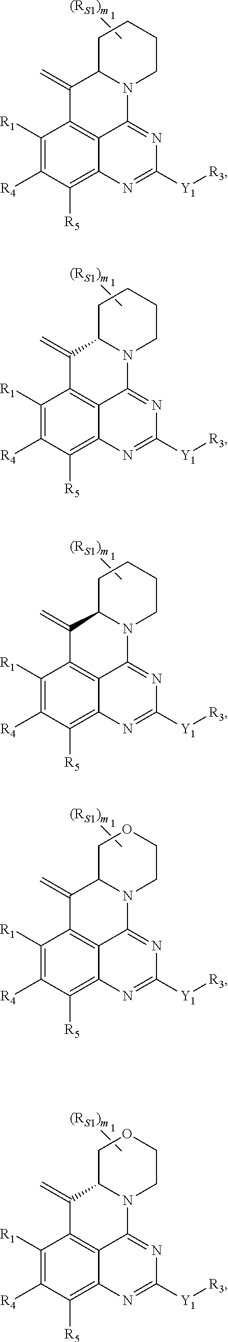

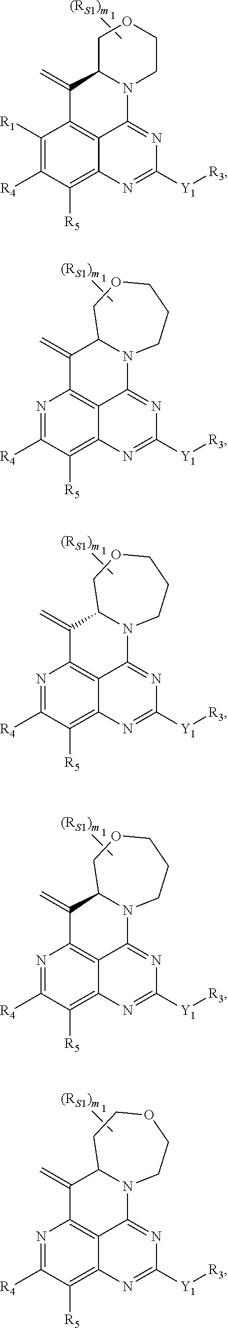

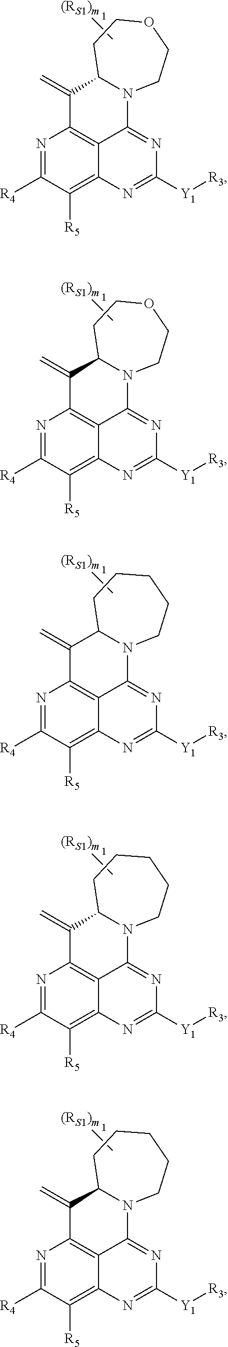

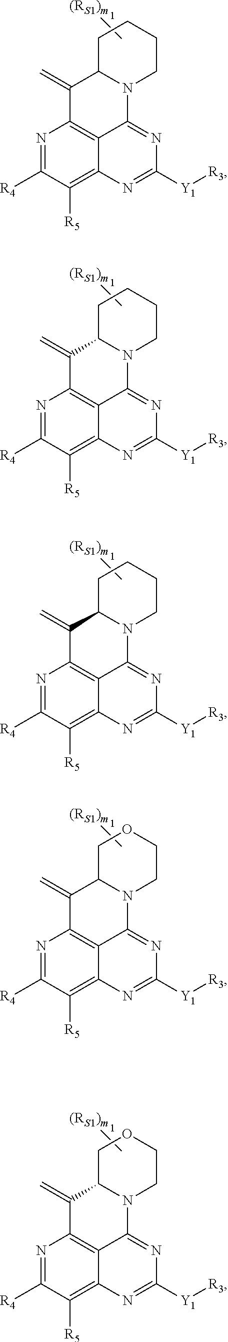

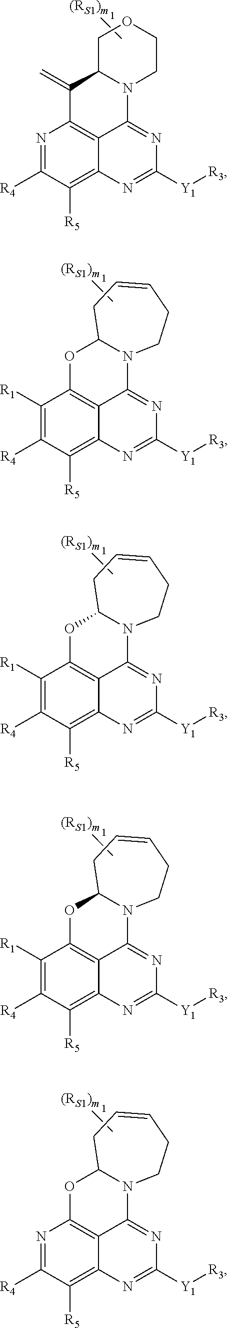

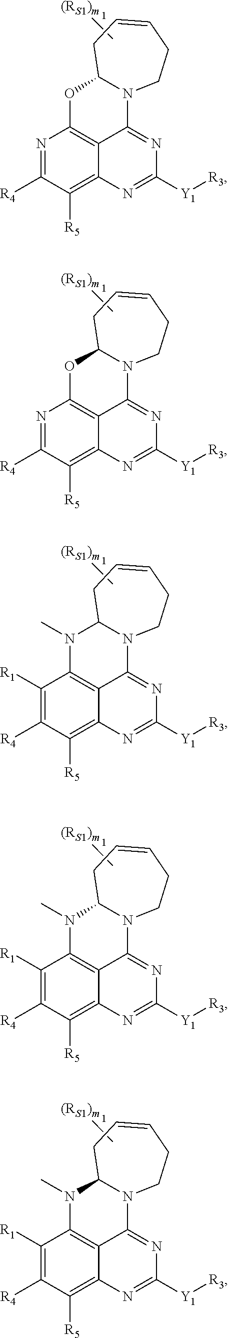

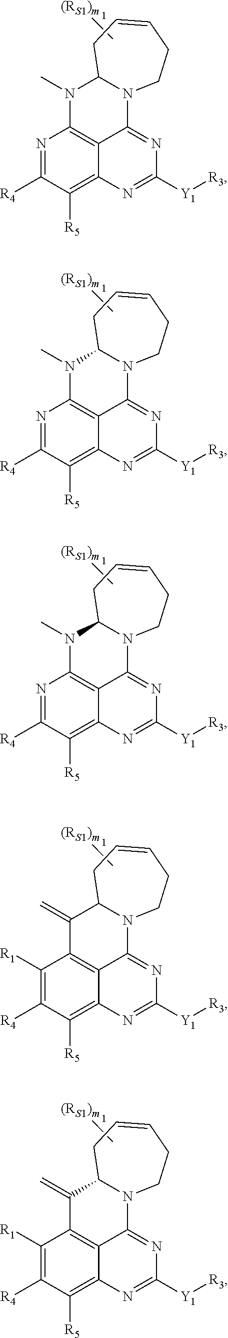

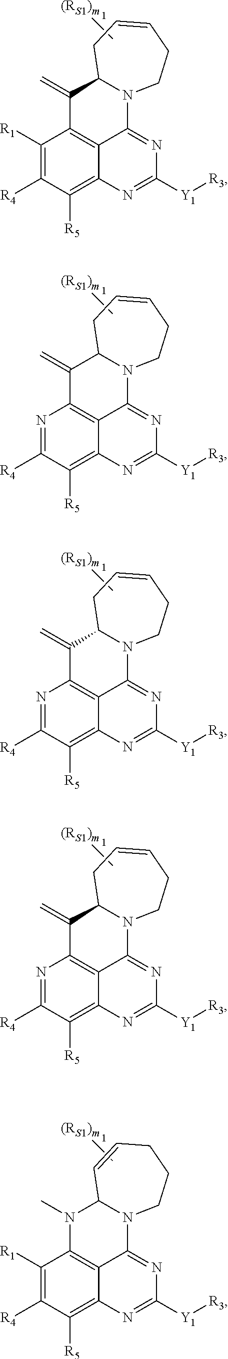

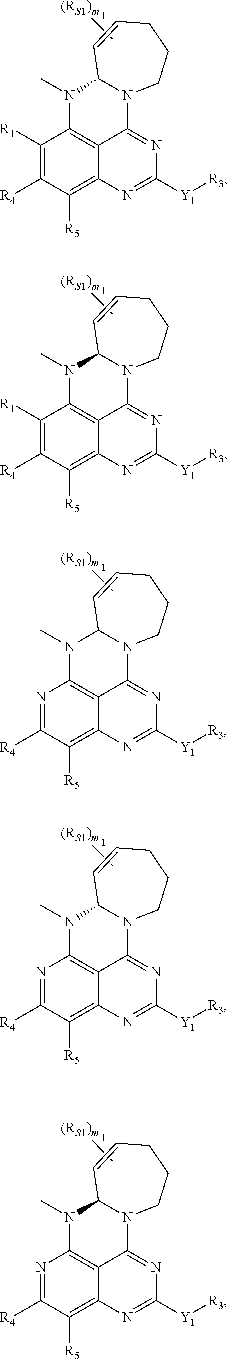

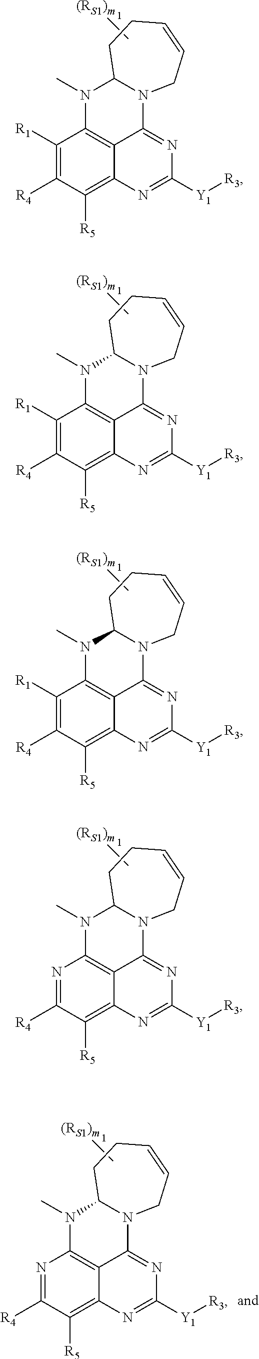

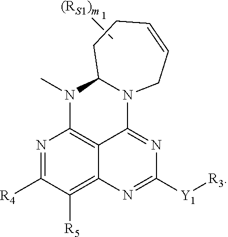

13. The compound of claim 1, wherein, Y₁ is O.

14. The compound of claim 1, wherein 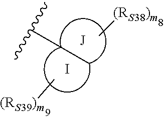
is selected from the group consisting of 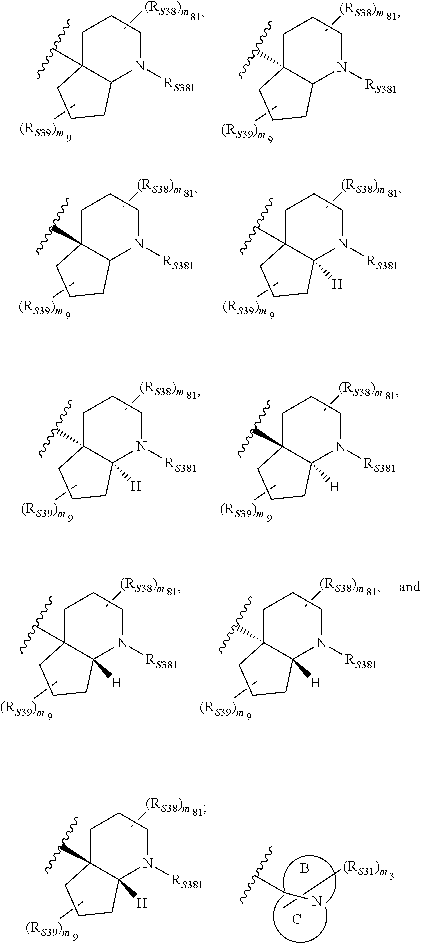
is selected from the group consisting of 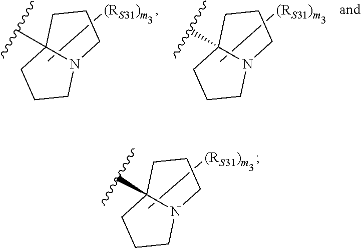
R_{S381} is hydrogen or R_{S38};
m₈₁ is selected from the group consisting of 0, 1, 2, 3, 4, 5, 6, 7, and 8; 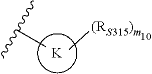
is selected from the group consisting of 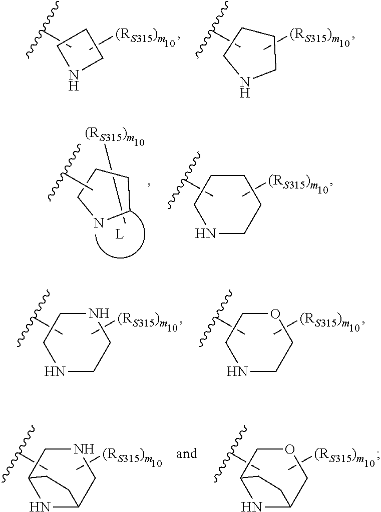
wherein, ring L is a 4-6 membered heterocyclic ring optionally further containing 1 or 2 heteroatoms selected from the group consisting of N and O.

15. The compound of claim 1, wherein, 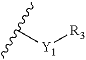
is selected from the group consisting of:

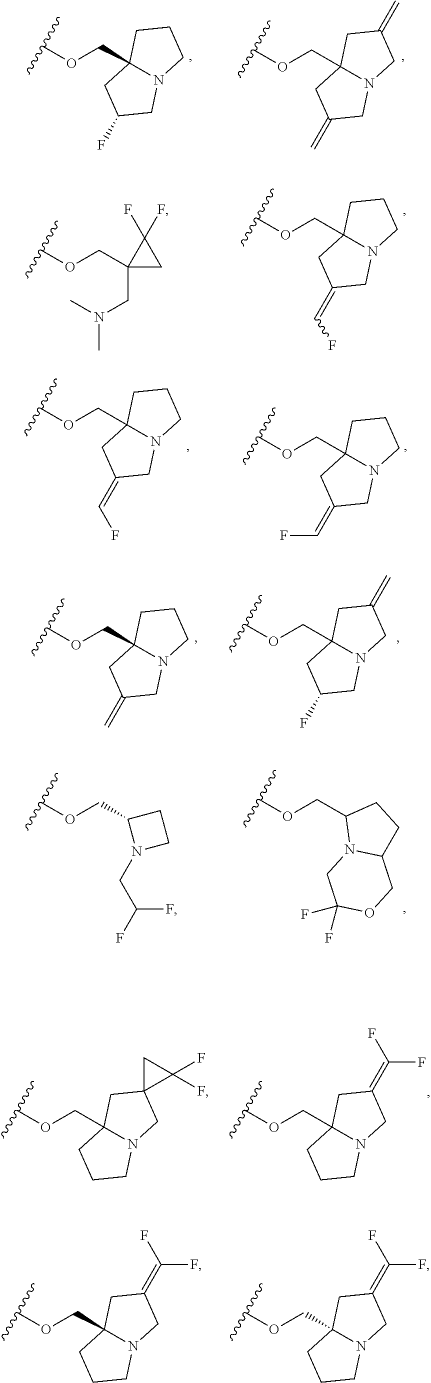

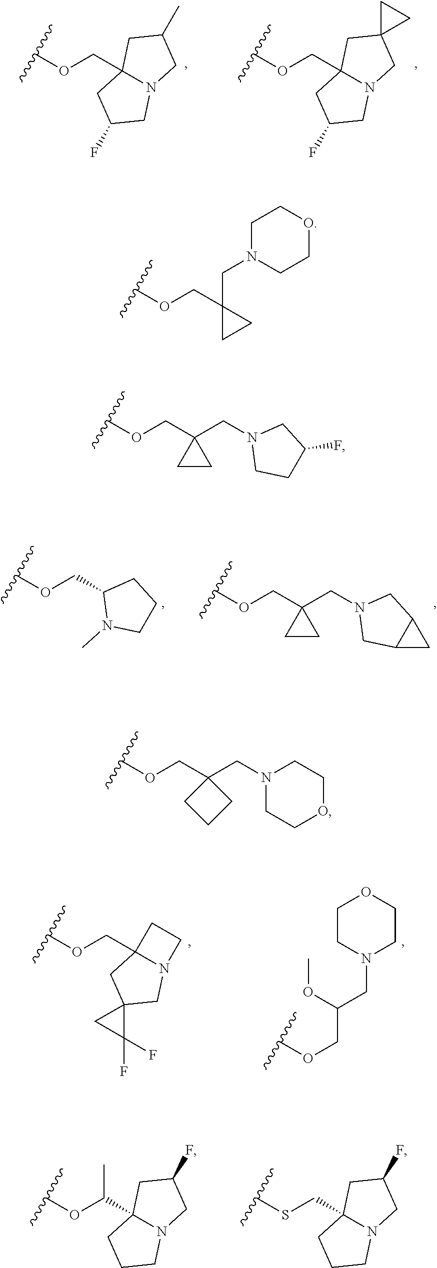

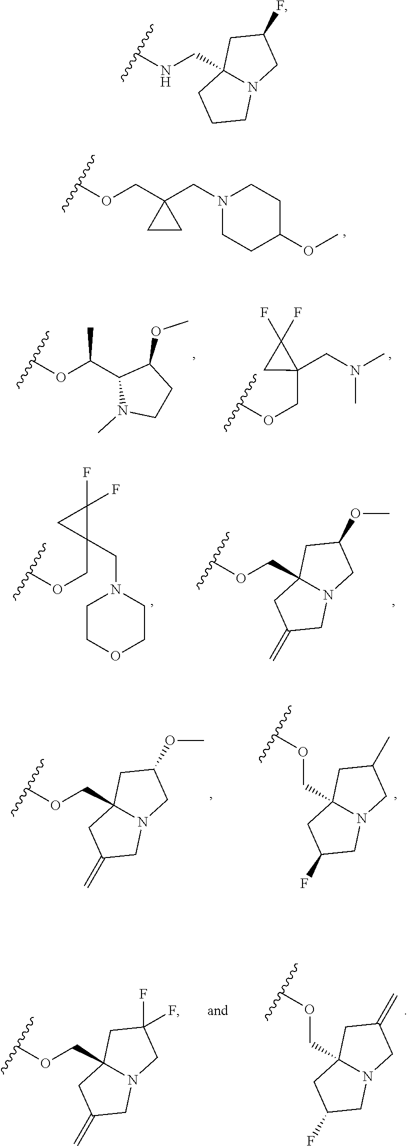

16. The compound of claim 1, wherein, R₄ is selected from the group consisting of 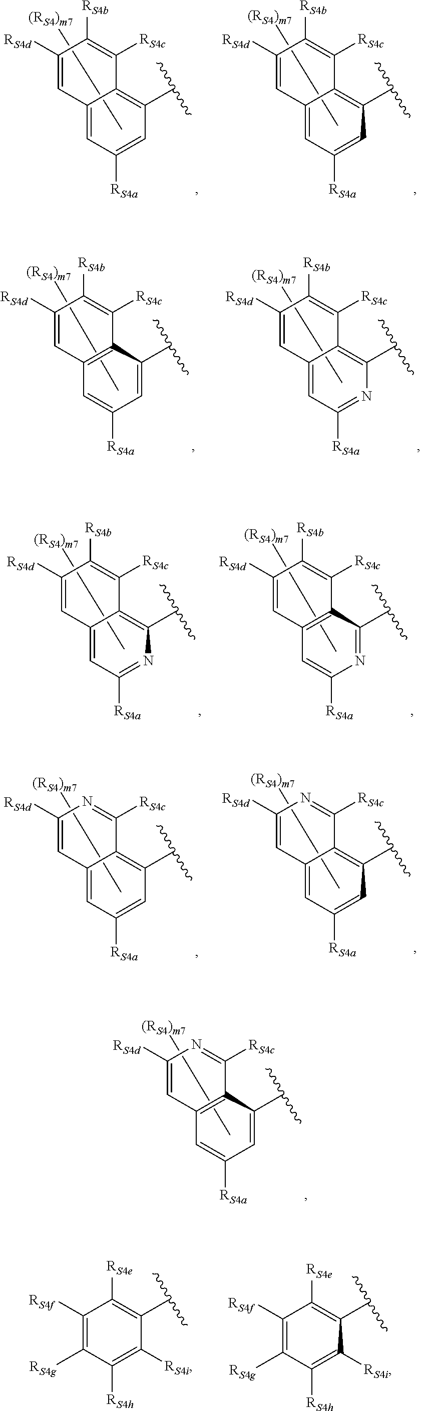 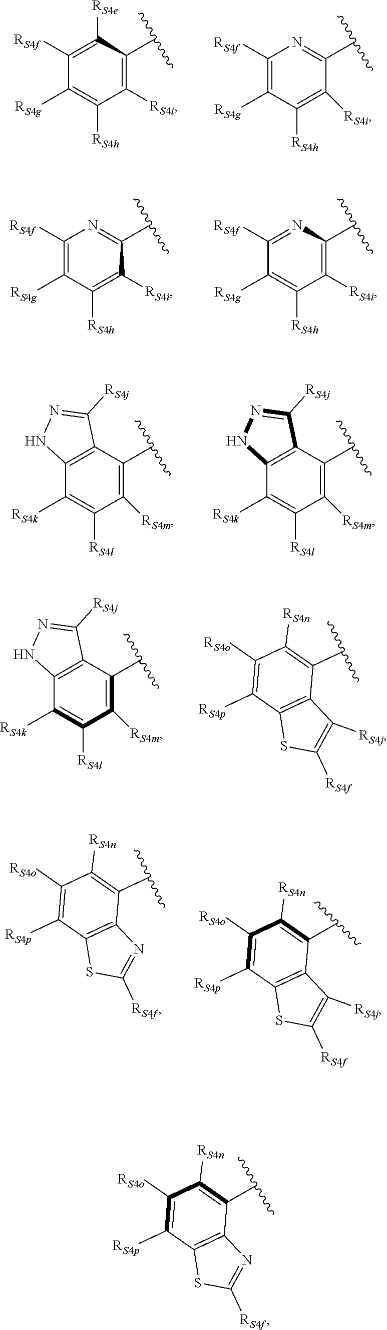 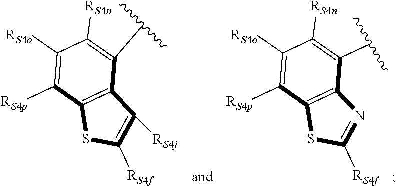
m₇ is selected from the group consisting of 0, 1, 2, and 3;
R_{S4a} at each occurrence is independently selected from the group consisting of —OH and —NH₂;
R_{S4b} at each occurrence is independently selected from the group consisting of hydrogen, deuterium, and halogen;
R_{S4c} at each occurrence is independently selected from the group consisting of hydrogen, deuterium, —C₁₋₃alkyl, —C₂₋₃alkenyl and —C₂₋₃alkynyl;
R_{S4d} at each occurrence is independently selected from the group consisting of hydrogen, deuterium and halogen;
R_{S4e} at each occurrence is independently selected from the group consisting of hydrogen, deuterium, halogen, —C₁₋₃alkyl and haloC₁₋₃alkyl;
R_{S4f} at each occurrence is independently selected from the group consisting of —OH and —NH₂;
R_{S4g} at each occurrence is independently selected from the group consisting of hydrogen, deuterium, halogen, —C₁₋₃alkyl and haloC₁₋₃alkyl;
R_{S4h} at each occurrence is independently selected from the group consisting of hydrogen, deuterium, halogen, —C₁₋₃alkyl and haloC₁₋₃alkyl;
R_{S4i} at each occurrence is independently selected from the group consisting of hydrogen, deuterium, halogen, —C₁₋₃alkyl and haloC₁₋₃alkyl;
R_{S4j} at each occurrence is independently selected from the group consisting of hydrogen, deuterium, halogen, —CN, —C₁₋₃alkyl, haloC₁₋₃alkyl and —OhaloC₁₋₃alkyl;
R_{S4k} at each occurrence is independently selected from the group consisting of hydrogen, deuterium, halogen, —CN, —C₁₋₃alkyl, haloC₁₋₃alkyl and —OhaloC₁₋₃alkyl;
R_{S4l} at each occurrence is independently selected from the group consisting of hydrogen, deuterium, halogen, —CN, —C₁₋₃alkyl, haloC₁₋₃alkyl and —OhaloC₁₋₃alkyl;
R_{S4m} at each occurrence is independently selected from the group consisting of hydrogen, deuterium, halogen, —CN, —C₁₋₃alkyl, haloC₁₋₃alkyl and —OhaloC₁₋₃alkyl;
R_{S4n} at each occurrence is independently selected from the group consisting of hydrogen, deuterium, halogen, —C₁₋₃alkyl and haloC₁₋₃alkyl;
Rₛ₄ₒ at each occurrence is independently selected from the group consisting of hydrogen, deuterium, halogen, —C₁₋₃alkyl and haloC₁₋₃alkyl;
R_{S4p} at each occurrence is independently selected from the group consisting of hydrogen, deuterium, halogen, —C₁₋₃alkyl and haloC₁₋₃alkyl.

17. The compound of claim 16, wherein, R₄ is selected from the group consisting of:

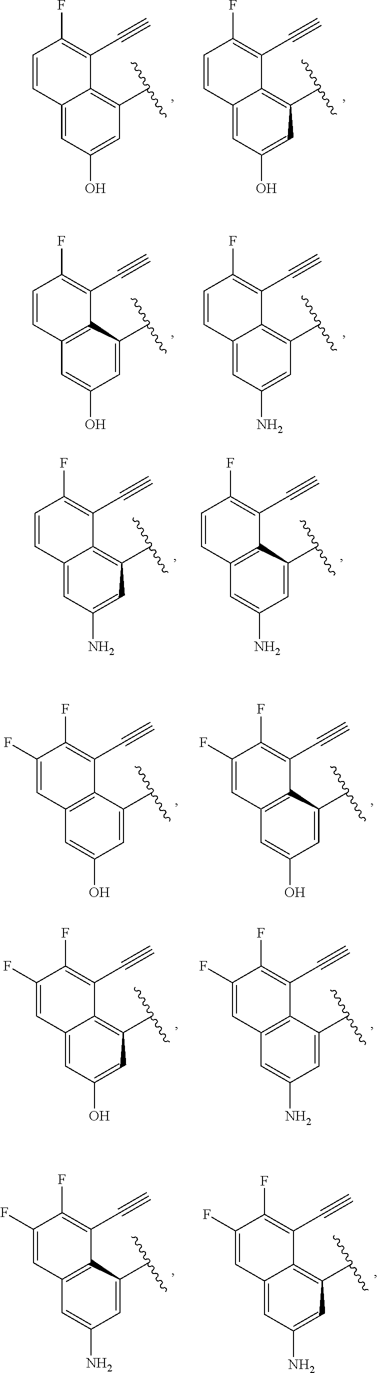

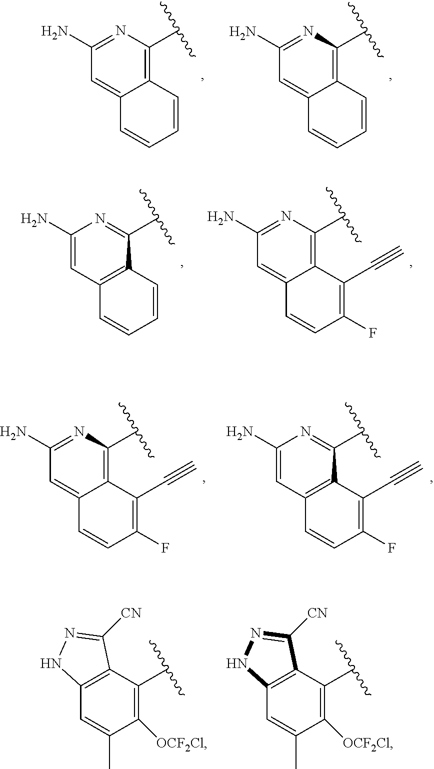

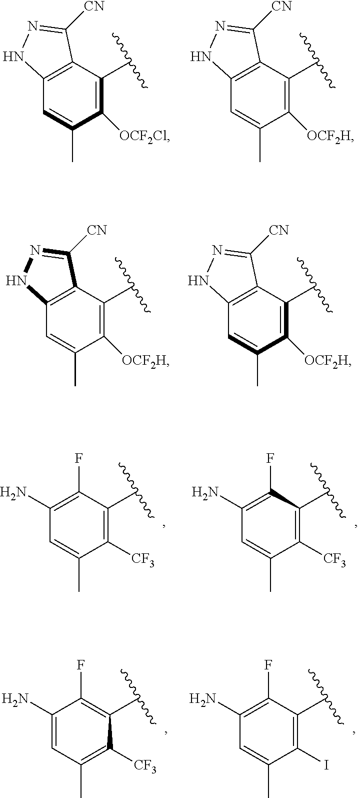

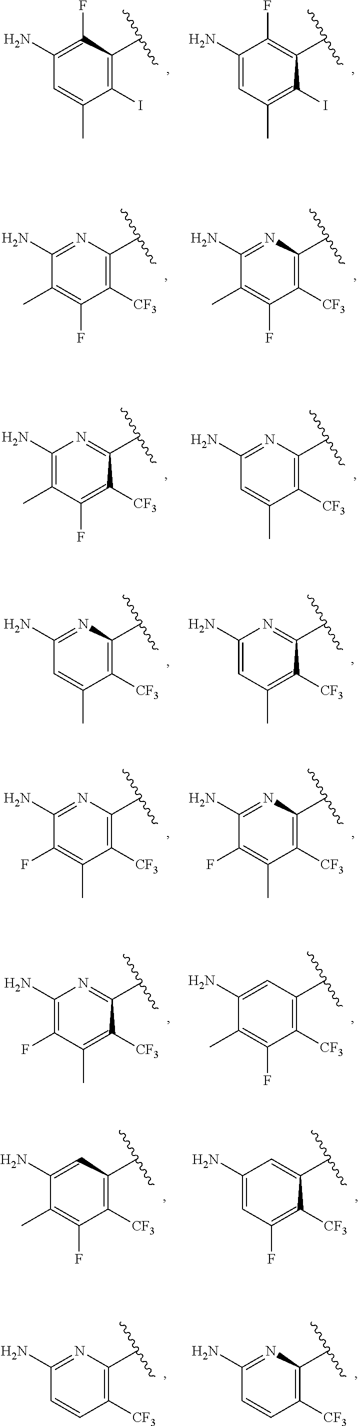

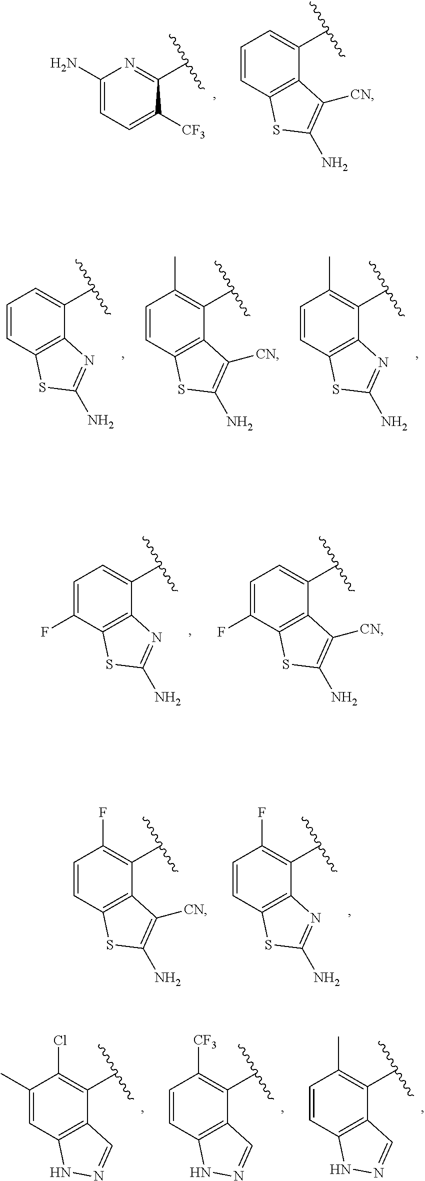

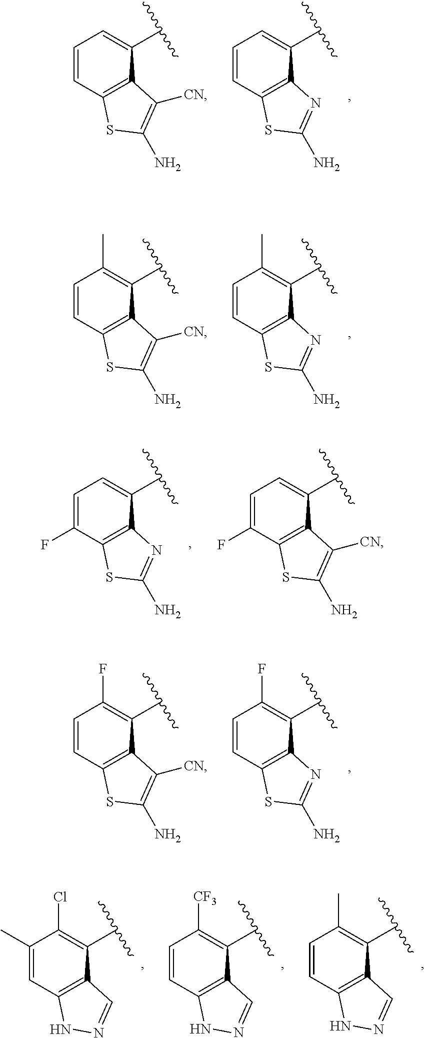

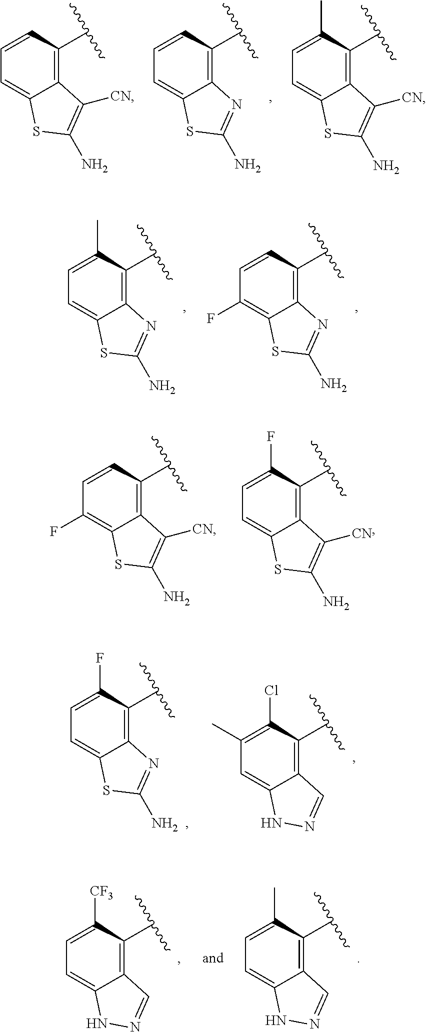

18. (canceled)

19. The compound of claim 1, wherein, R₅ is —F.

20. The compound of claim 1, wherein, the compound is selected from the group consisting of any one of the following compounds:

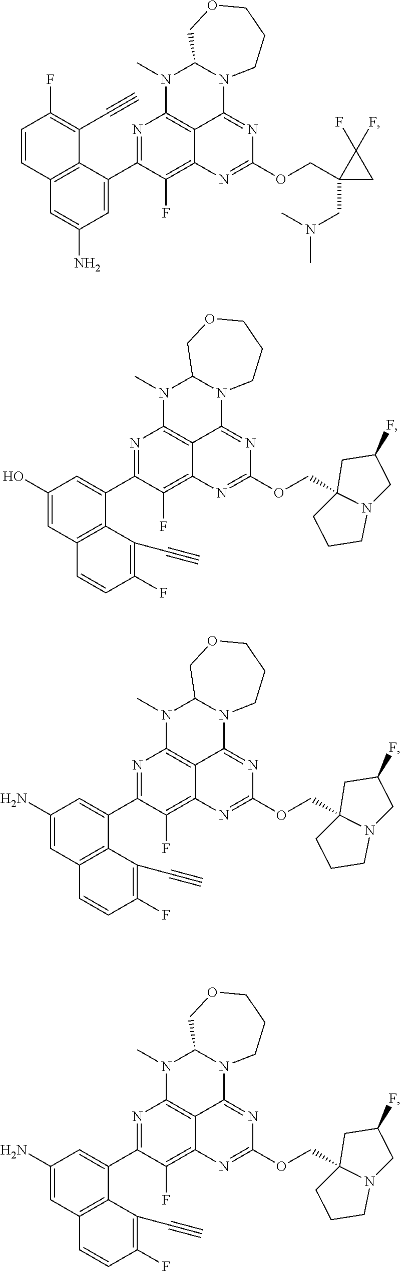 [SMILES: C#Cc1c(F)ccc2cc(N)cc(-c3nc4c5c(nc(OC[C@]6(CN(C)C)CC6(F)F)nc5c3F)N3CCCOC[C@H]3N4C)c12 ; C#Cc1c(F)ccc2cc(O)cc(-c3nc4c5c(nc(OC[C@@]67CCCN6C[C@H](F)C7)nc5c3F)N3CCCOCC3N4C)c12 ; C#Cc1c(F)ccc2cc(N)cc(-c3nc4c5c(nc(OC[C@@]67CCCN6C[C@H](F)C7)nc5c3F)N3CCCOCC3N4C)c12 ; C#Cc1c(F)ccc2cc(N)cc(-c3nc4c5c(nc(OC[C@@]67CCCN6C[C@H](F)C7)nc5c3F)N3CCCOC[C@H]3N4C)c12]

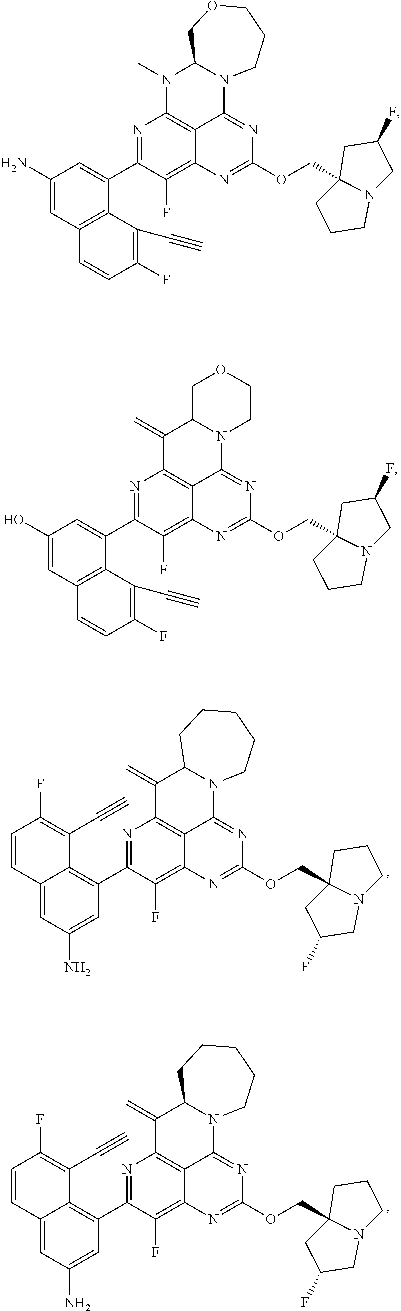 [SMILES: C#Cc1c(F)ccc2cc(N)cc(-c3nc4c5c(nc(OC[C@@]67CCCN6C[C@H](F)C7)nc5c3F)N3CCCOC[C@@H]3N4C)c12 ; C#Cc1c(F)ccc2cc(O)cc(-c3nc4c5c(nc(OC[C@@]67CCCN6C[C@H](F)C7)nc5c3F)N3CCOCC3C4=C)c12 ; C#Cc1c(F)ccc2cc(N)cc(-c3nc4c5c(nc(OC[C@@]67CCCN6C[C@H](F)C7)nc5c3F)N3CCCCCC3C4=C)c12 ; C#Cc1c(F)ccc2cc(N)cc(-c3nc4c5c(nc(OC[C@@]67CCCN6C[C@H](F)C7)nc5c3F)N3CCCCC[C@@H]3C4=C)c12]

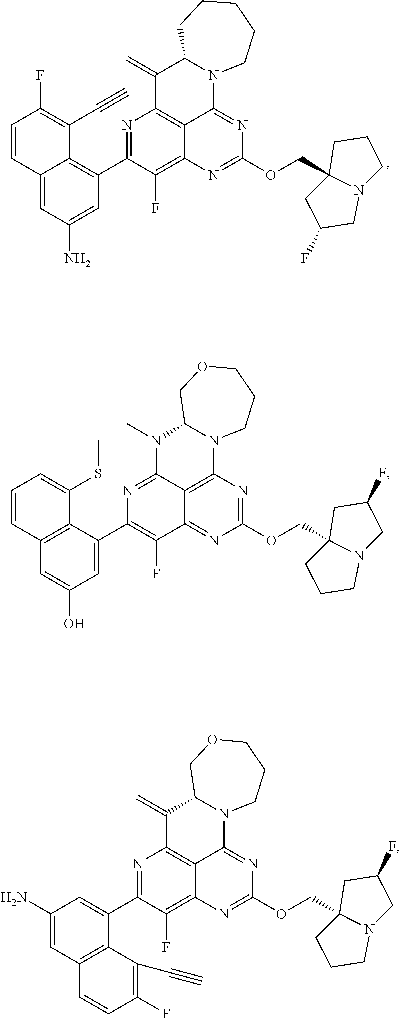 [SMILES: C#Cc1c(F)ccc2cc(N)cc(-c3nc4c5c(nc(OC[C@@]67CCCN6C[C@H](F)C7)nc5c3F)N3CCCCC[C@H]3C4=C)c12 ; CSc1cccc2cc(O)cc(-c3nc4c5c(nc(OC[C@@]67CCCN6C[C@H](F)C7)nc5c3F)N3CCCOC[C@H]3N4C)c12 ; C#Cc1c(F)ccc2cc(N)cc(-c3nc4c5c(nc(OC[C@@]67CCCN6C[C@H](F)C7)nc5c3F)N3CCCOC[C@H]3C4=C)c12]

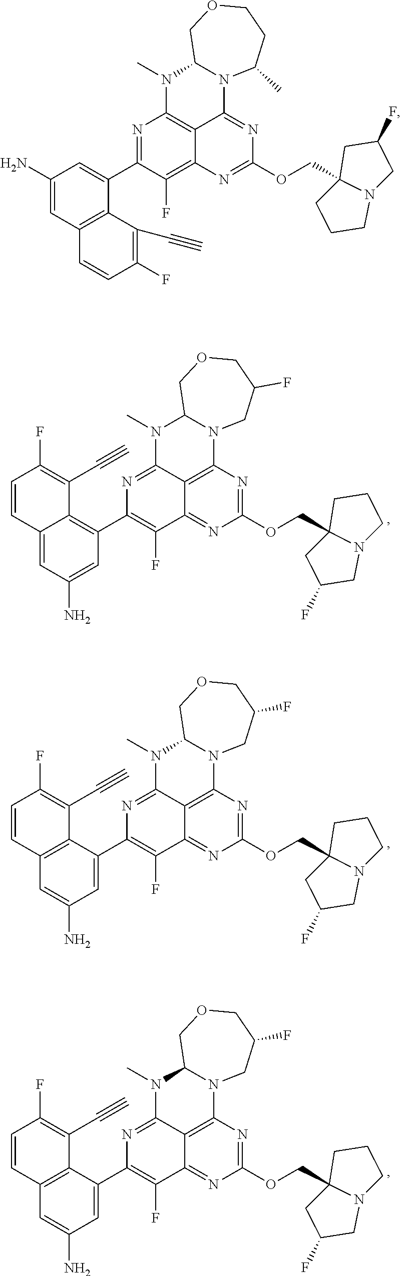 [SMILES: C#Cc1c(F)ccc2cc(N)cc(-c3nc4c5c(nc(OC[C@@]67CCCN6C[C@H](F)C7)nc5c3F)N3[C@@H](C)CCOC[C@H]3N4C)c12 ; C#Cc1c(F)ccc2cc(N)cc(-c3nc4c5c(nc(OC[C@@]67CCCN6C[C@H](F)C7)nc5c3F)N3CC(F)COCC3N4C)c12 ; C#Cc1c(F)ccc2cc(N)cc(-c3nc4c5c(nc(OC[C@@]67CCCN6C[C@H](F)C7)nc5c3F)N3C[C@@H](F)COC[C@H]3N4C)c12 ; C#Cc1c(F)ccc2cc(N)cc(-c3nc4c5c(nc(OC[C@@]67CCCN6C[C@H](F)C7)nc5c3F)N3C[C@@H](F)COC[C@@H]3N4C)c12]

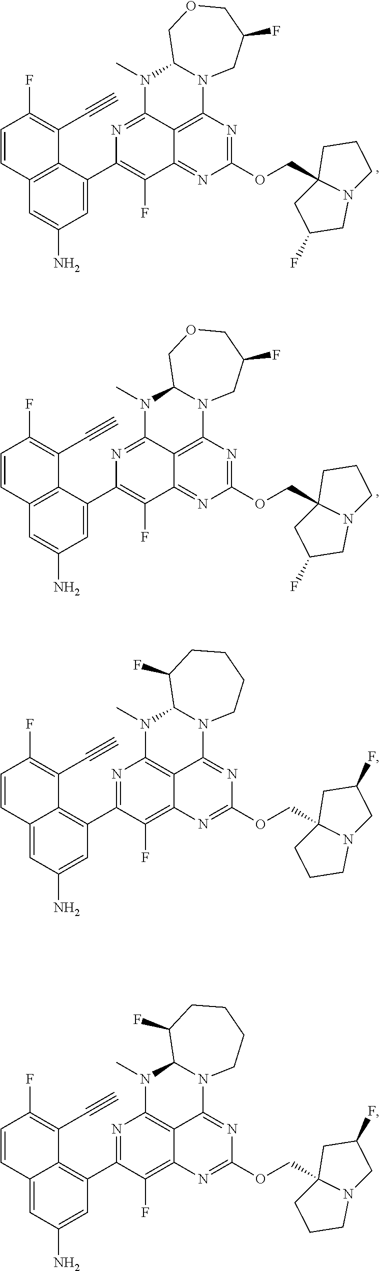 [SMILES: C#Cc1c(F)ccc2cc(N)cc(-c3nc4c5c(nc(OC[C@@]67CCCN6C[C@H](F)C7)nc5c3F)N3C[C@H](F)COC[C@H]3N4C)c12 ; C#Cc1c(F)ccc2cc(N)cc(-c3nc4c5c(nc(OC[C@@]67CCCN6C[C@H](F)C7)nc5c3F)N3C[C@H](F)COC[C@@H]3N4C)c12 ; C#Cc1c(F)ccc2cc(N)cc(-c3nc4c5c(nc(OC[C@@]67CCCN6C[C@H](F)C7)nc5c3F)N3CCCC[C@H](F)[C@H]3N4C)c12 ; C#Cc1c(F)ccc2cc(N)cc(-c3nc4c5c(nc(OC[C@@]67CCCN6C[C@H](F)C7)nc5c3F)N3CCCC[C@H](F)[C@@H]3N4C)c12]

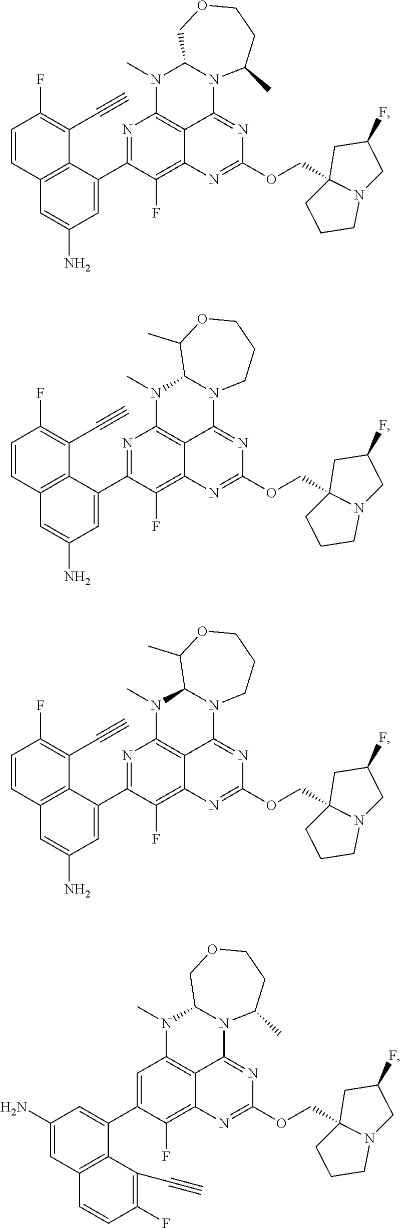 [SMILES: C#Cc1c(F)ccc2cc(N)cc(-c3nc4c5c(nc(OC[C@@]67CCCN6C[C@H](F)C7)nc5c3F)N3[C@H](C)CCOC[C@H]3N4C)c12 ; C#Cc1c(F)ccc2cc(N)cc(-c3nc4c5c(nc(OC[C@@]67CCCN6C[C@H](F)C7)nc5c3F)N3CCCOC(C)[C@H]3N4C)c12 ; C#Cc1c(F)ccc2cc(N)cc(-c3nc4c5c(nc(OC[C@@]67CCCN6C[C@H](F)C7)nc5c3F)N3CCCOC(C)[C@@H]3N4C)c12 ; C#Cc1c(F)ccc2cc(N)cc(-c3cc4c5c(nc(OC[C@@]67CCCN6C[C@H](F)C7)nc5c3F)N3[C@@H](C)CCOC[C@H]3N4C)c12]

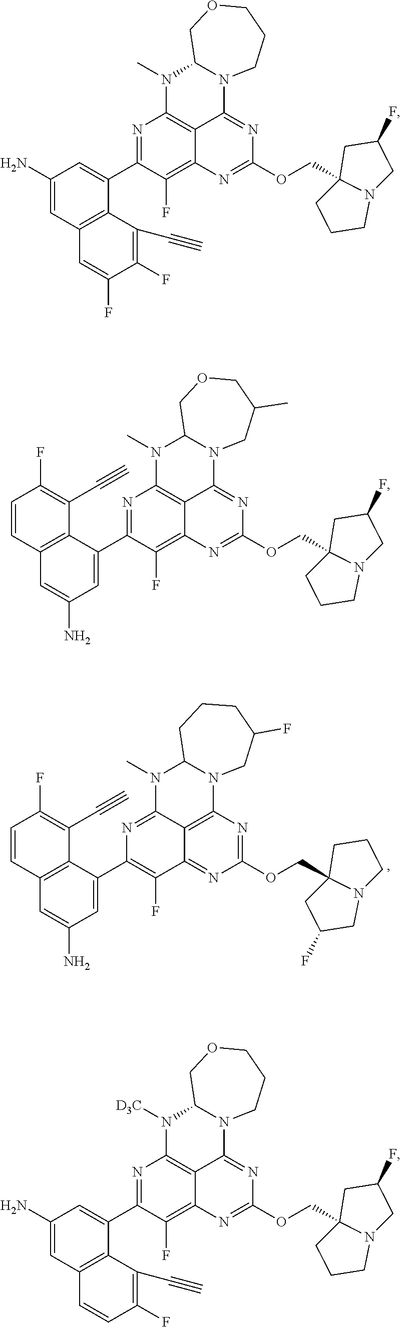 [SMILES: C#Cc1c(F)c(F)cc2cc(N)cc(-c3nc4c5c(nc(OC[C@@]67CCCN6C[C@H](F)C7)nc5c3F)N3CCCOC[C@H]3N4C)c12 ; C#Cc1c(F)ccc2cc(N)cc(-c3nc4c5c(nc(OC[C@@]67CCCN6C[C@H](F)C7)nc5c3F)N3CC(C)COCC3N4C)c12 ; C#Cc1c(F)ccc2cc(N)cc(-c3nc4c5c(nc(OC[C@@]67CCCN6C[C@H](F)C7)nc5c3F)N3CC(F)CCCC3N4C)c12 ; [2H]C([2H])([2H])N1c2nc(-c3cc(N)cc4ccc(F)c(C#C)c34)c(F)c3nc(OC[C@@]45CCCN4C[C@H](F)C5)nc(c23)N2CCCOC[C@H]21]

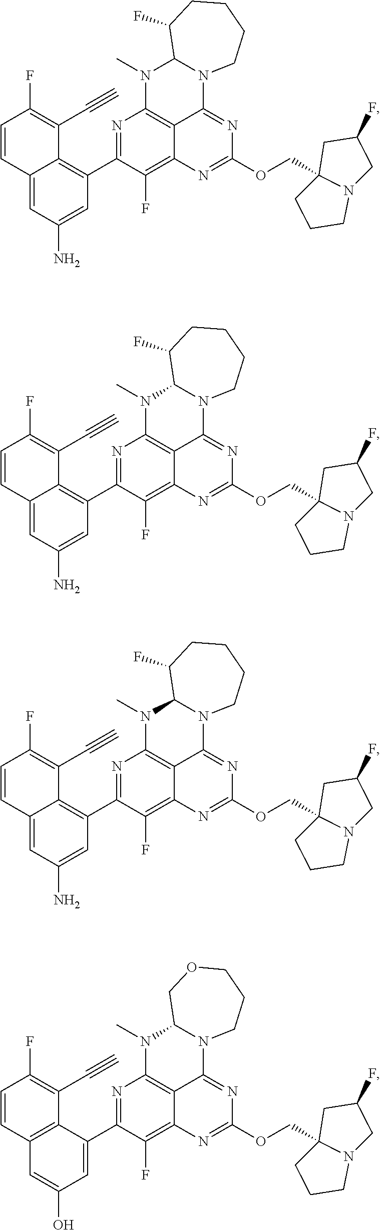 [SMILES: C#Cc1c(F)ccc2cc(N)cc(-c3nc4c5c(nc(OC[C@@]67CCCN6C[C@H](F)C7)nc5c3F)N3CCCC[C@@H](F)C3N4C)c12 ; C#Cc1c(F)ccc2cc(N)cc(-c3nc4c5c(nc(OC[C@@]67CCCN6C[C@H](F)C7)nc5c3F)N3CCCC[C@@H](F)[C@H]3N4C)c12 ; C#Cc1c(F)ccc2cc(N)cc(-c3nc4c5c(nc(OC[C@@]67CCCN6C[C@H](F)C7)nc5c3F)N3CCCC[C@@H](F)[C@@H]3N4C)c12 ; C#Cc1c(F)ccc2cc(O)cc(-c3nc4c5c(nc(OC[C@@]67CCCN6C[C@H](F)C7)nc5c3F)N3CCCOC[C@H]3N4C)c12]

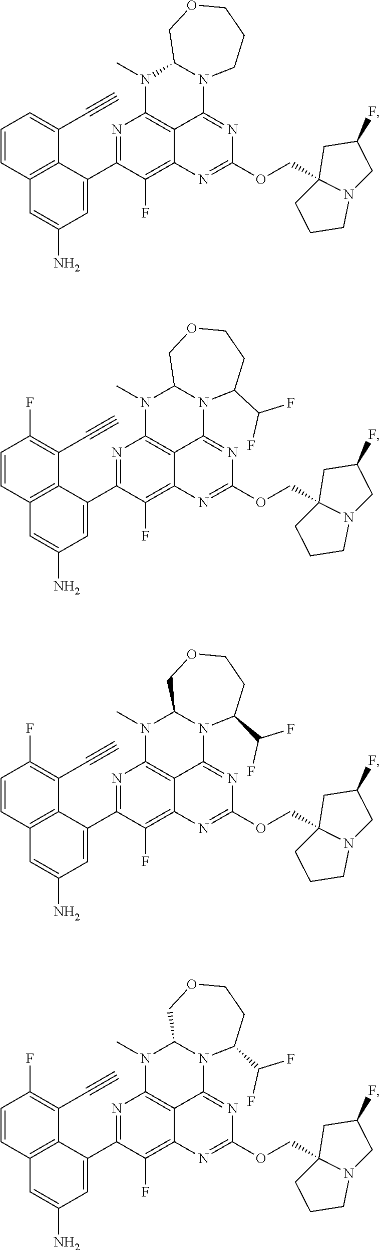 [SMILES: C#Cc1cccc2cc(N)cc(-c3nc4c5c(nc(OC[C@@]67CCCN6C[C@H](F)C7)nc5c3F)N3CCCOC[C@H]3N4C)c12 ; C#Cc1c(F)ccc2cc(N)cc(-c3nc4c5c(nc(OC[C@@]67CCCN6C[C@H](F)C7)nc5c3F)N3C(C(F)F)CCOCC3N4C)c12 ; C#Cc1c(F)ccc2cc(N)cc(-c3nc4c5c(nc(OC[C@@]67CCCN6C[C@H](F)C7)nc5c3F)N3[C@H](COCC[C@H]3C(F)F)N4C)c12 ; C#Cc1c(F)ccc2cc(N)cc(-c3nc4c5c(nc(OC[C@@]67CCCN6C[C@H](F)C7)nc5c3F)N3[C@@H](C(F)F)CCOC[C@H]3N4C)c12]

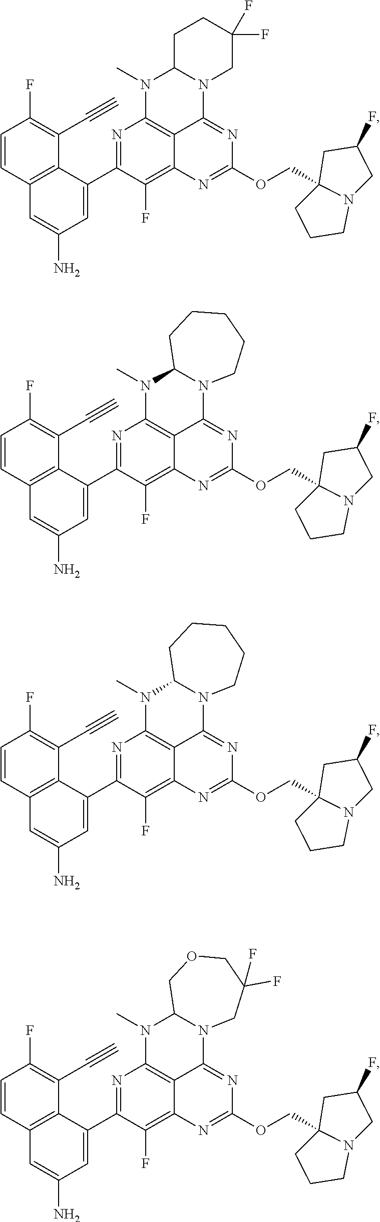 [SMILES: C#Cc1c(F)ccc2cc(N)cc(-c3nc4c5c(nc(OC[C@@]67CCCN6C[C@H](F)C7)nc5c3F)N3CC(F)(F)CCC3N4C)c12 ; C#Cc1c(F)ccc2cc(N)cc(-c3nc4c5c(nc(OC[C@@]67CCCN6C[C@H](F)C7)nc5c3F)N3CCCCC[C@@H]3N4C)c12 ; C#Cc1c(F)ccc2cc(N)cc(-c3nc4c5c(nc(OC[C@@]67CCCN6C[C@H](F)C7)nc5c3F)N3CCCCC[C@H]3N4C)c12 ; C#Cc1c(F)ccc2cc(N)cc(-c3nc4c5c(nc(OC[C@@]67CCCN6C[C@H](F)C7)nc5c3F)N3CC(F)(F)COCC3N4C)c12]

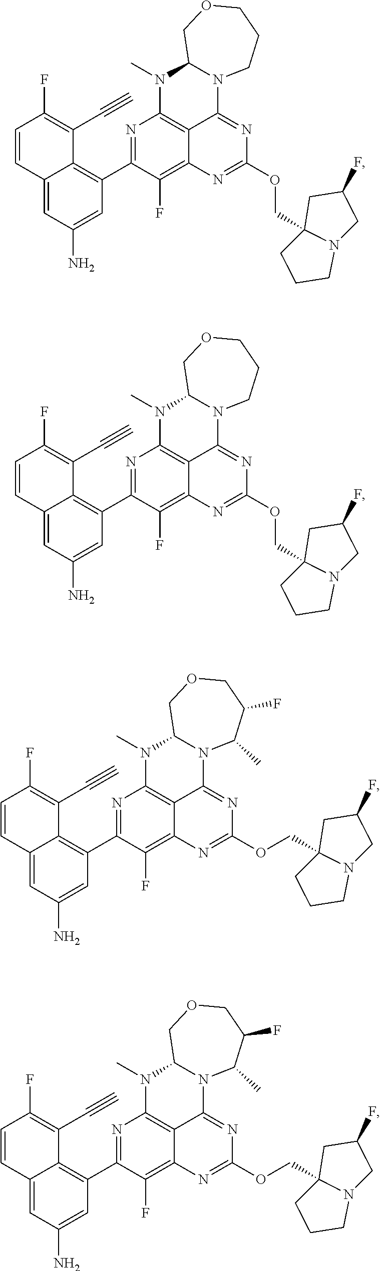 [SMILES: C#Cc1c(F)ccc2cc(N)cc(-c3nc4c5c(nc(OC[C@@]67CCCN6C[C@H](F)C7)nc5c3F)N3CCCOC[C@@H]3N4C)c12 ; C#Cc1c(F)ccc2cc(N)cc(-c3nc4c5c(nc(OC[C@@]67CCCN6C[C@H](F)C7)nc5c3F)N3CCCOC[C@H]3N4C)c12 ; C#Cc1c(F)ccc2cc(N)cc(-c3nc4c5c(nc(OC[C@@]67CCCN6C[C@H](F)C7)nc5c3F)N3[C@@H](C)[C@@H](F)COC[C@H]3N4C)c12 ; C#Cc1c(F)ccc2cc(N)cc(-c3nc4c5c(nc(OC[C@@]67CCCN6C[C@H](F)C7)nc5c3F)N3[C@@H](C)[C@H](F)COC[C@H]3N4C)c12]

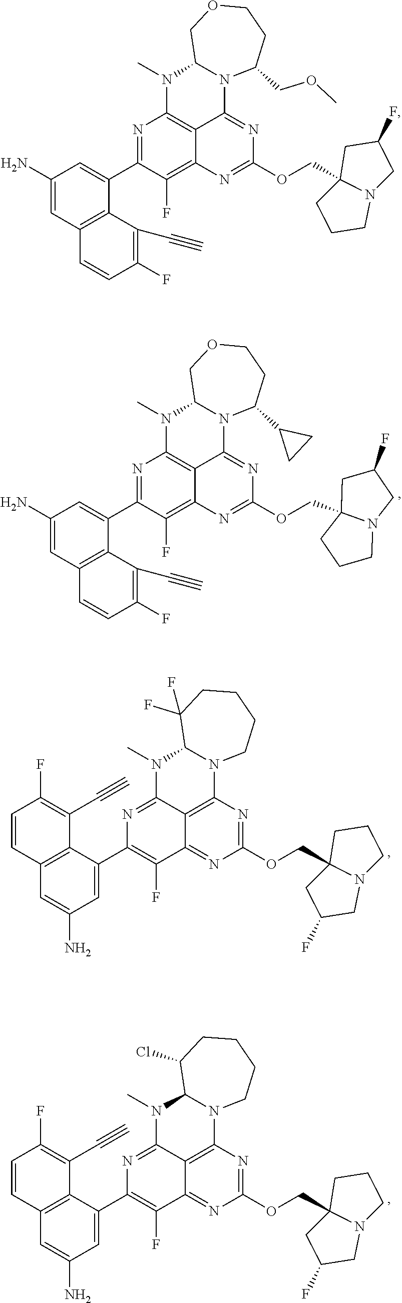 [SMILES: C#Cc1c(F)ccc2cc(N)cc(-c3nc4c5c(nc(OC[C@@]67CCCN6C[C@H](F)C7)nc5c3F)N3[C@@H](COC)CCOC[C@H]3N4C)c12 ; C#Cc1c(F)ccc2cc(N)cc(-c3nc4c5c(nc(OC[C@@]67CCCN6C[C@H](F)C7)nc5c3F)N3[C@@H](C5CC5)CCOC[C@H]3N4C)c12 ; C#Cc1c(F)ccc2cc(N)cc(-c3nc4c5c(nc(OC[C@@]67CCCN6C[C@H](F)C7)nc5c3F)N3CCCCC(F)(F)[C@H]3N4C)c12 ; C#Cc1c(F)ccc2cc(N)cc(-c3nc4c5c(nc(OC[C@@]67CCCN6C[C@H](F)C7)nc5c3F)N3CCCC[C@@H](Cl)[C@@H]3N4C)c12]

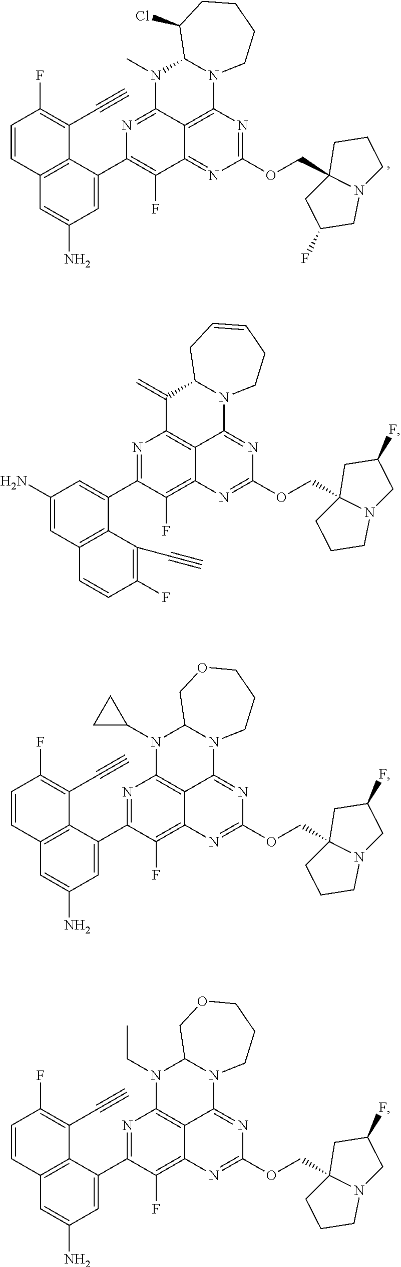 [SMILES: C#Cc1c(F)ccc2cc(N)cc(-c3nc4c5c(nc(OC[C@@]67CCCN6C[C@H](F)C7)nc5c3F)N3CCCC[C@H](Cl)[C@H]3N4C)c12 ; C#Cc1c(F)ccc2cc(N)cc(-c3nc4c5c(nc(OC[C@@]67CCCN6C[C@H](F)C7)nc5c3F)N3CCC=CC[C@H]3C4=C)c12 ; C#Cc1c(F)ccc2cc(N)cc(-c3nc4c5c(nc(OC[C@@]67CCCN6C[C@H](F)C7)nc5c3F)N3CCCOCC3N4C3CC3)c12 ; C#Cc1c(F)ccc2cc(N)cc(-c3nc4c5c(nc(OC[C@@]67CCCN6C[C@H](F)C7)nc5c3F)N3CCCOCC3N4CC)c12]

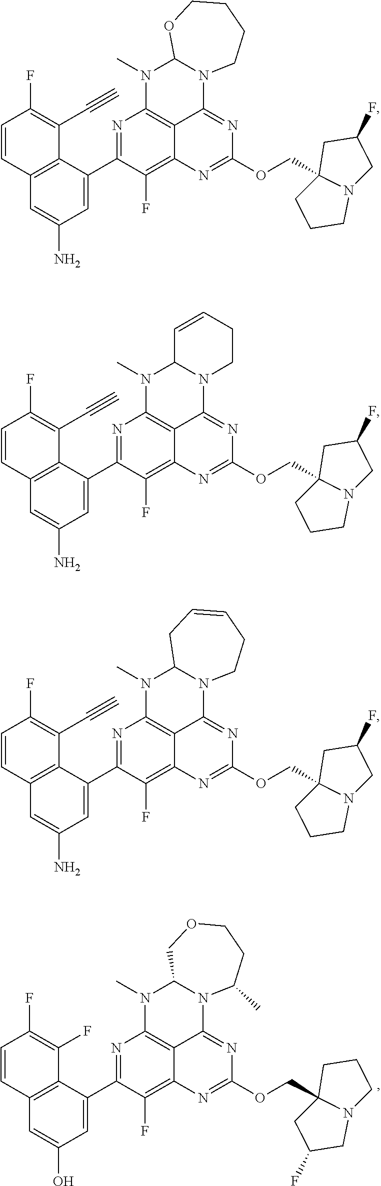 [SMILES: C#Cc1c(F)ccc2cc(N)cc(-c3nc4c5c(nc(OC[C@@]67CCCN6C[C@H](F)C7)nc5c3F)N3CCCCOC3N4C)c12 ; C#Cc1c(F)ccc2cc(N)cc(-c3nc4c5c(nc(OC[C@@]67CCCN6C[C@H](F)C7)nc5c3F)N3CCC=CC3N4C)c12 ; C#Cc1c(F)ccc2cc(N)cc(-c3nc4c5c(nc(OC[C@@]67CCCN6C[C@H](F)C7)nc5c3F)N3CCC=CCC3N4C)c12 ; C[C@H]1CCOC[C@H]2N(C)c3nc(-c4cc(O)cc5ccc(F)c(F)c45)c(F)c4nc(OC[C@@]56CCCN5C[C@H](F)C6)nc(c34)N12]

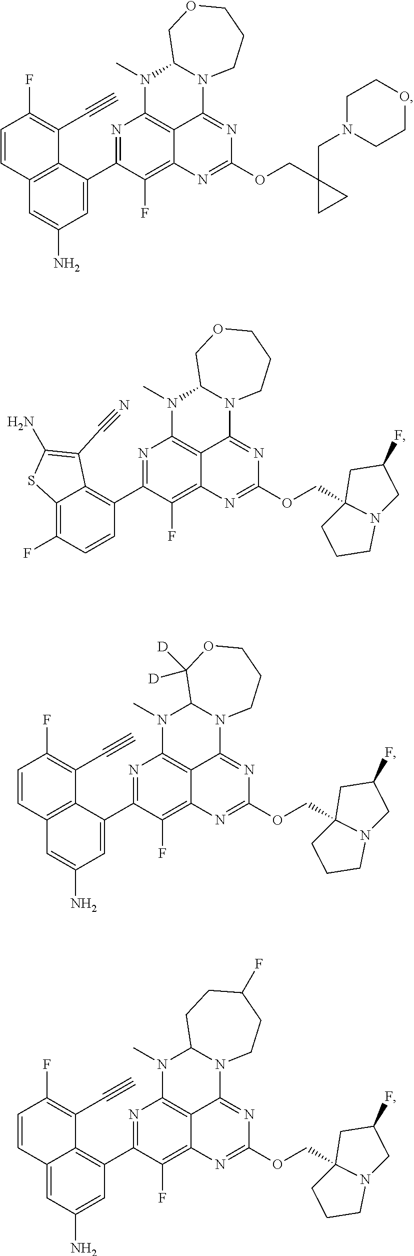

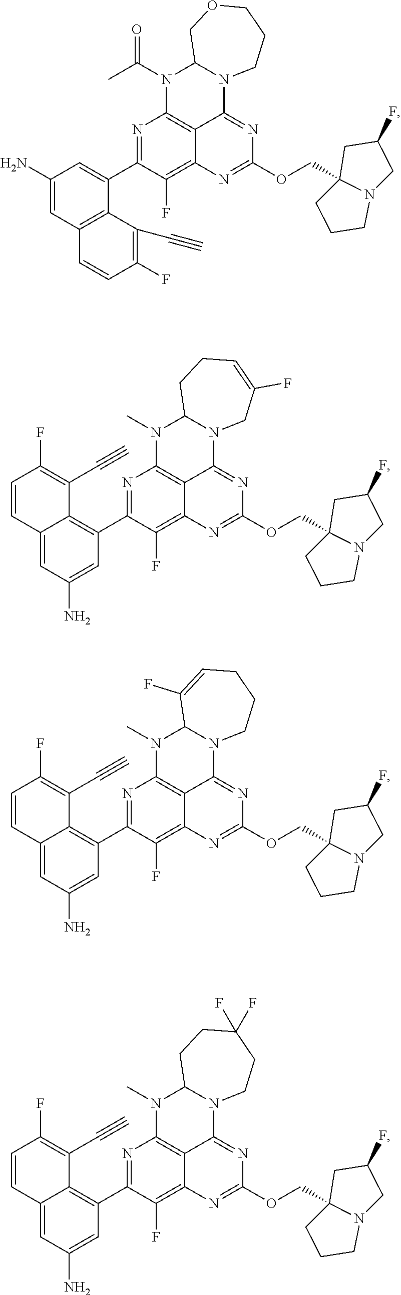 [SMILES: C#Cc1c(F)ccc2cc(N)cc(-c3nc4c5c(nc(OC[C@@]67CCCN6C[C@H](F)C7)nc5c3F)N3CCCOCC3N4C(C)=O)c12 ; C#Cc1c(F)ccc2cc(N)cc(-c3nc4c5c(nc(OC[C@@]67CCCN6C[C@H](F)C7)nc5c3F)N3CC(F)=CCCC3N4C)c12 ; C#Cc1c(F)ccc2cc(N)cc(-c3nc4c5c(nc(OC[C@@]67CCCN6C[C@H](F)C7)nc5c3F)N3CCCC=C(F)C3N4C)c12 ; C#Cc1c(F)ccc2cc(N)cc(-c3nc4c5c(nc(OC[C@@]67CCCN6C[C@H](F)C7)nc5c3F)N3CCC(F)(F)CCC3N4C)c12]

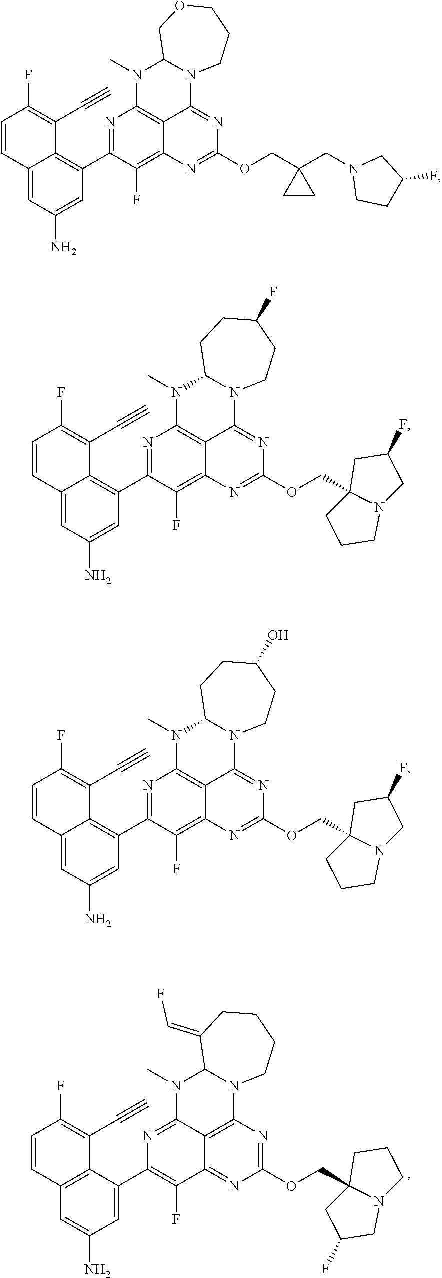 [SMILES: C#Cc1c(F)ccc2cc(N)cc(-c3nc4c5c(nc(OCC6(CN7CC[C@@H](F)C7)CC6)nc5c3F)N3CCCOCC3N4C)c12 ; C#Cc1c(F)ccc2cc(N)cc(-c3nc4c5c(nc(OC[C@@]67CCCN6C[C@H](F)C7)nc5c3F)N3CC[C@H](F)CC[C@H]3N4C)c12 ; C#Cc1c(F)ccc2cc(N)cc(-c3nc4c5c(nc(OC[C@@]67CCCN6C[C@H](F)C7)nc5c3F)N3CC[C@@H](O)CC[C@H]3N4C)c12 ; C#Cc1c(F)ccc2cc(N)cc(-c3nc4c5c(nc(OC[C@@]67CCCN6C[C@H](F)C7)nc5c3F)N3CCCC/C(=C\F)C3N4C)c12]

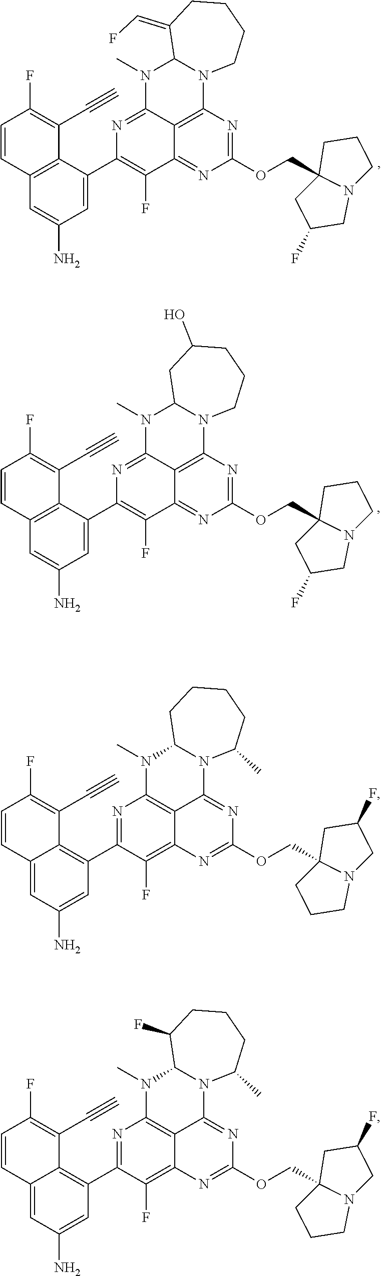 [SMILES: C#Cc1c(F)ccc2cc(N)cc(-c3nc4c5c(nc(OC[C@@]67CCCN6C[C@H](F)C7)nc5c3F)N3CCCC/C(=C/F)C3N4C)c12 ; C#Cc1c(F)ccc2cc(N)cc(-c3nc4c5c(nc(OC[C@@]67CCCN6C[C@H](F)C7)nc5c3F)N3CCCC(O)CC3N4C)c12 ; C#Cc1c(F)ccc2cc(N)cc(-c3nc4c5c(nc(OC[C@@]67CCCN6C[C@H](F)C7)nc5c3F)N3[C@@H](C)CCCC[C@H]3N4C)c12 ; C#Cc1c(F)ccc2cc(N)cc(-c3nc4c5c(nc(OC[C@@]67CCCN6C[C@H](F)C7)nc5c3F)N3[C@@H](C)CCC[C@H](F)[C@H]3N4C)c12]

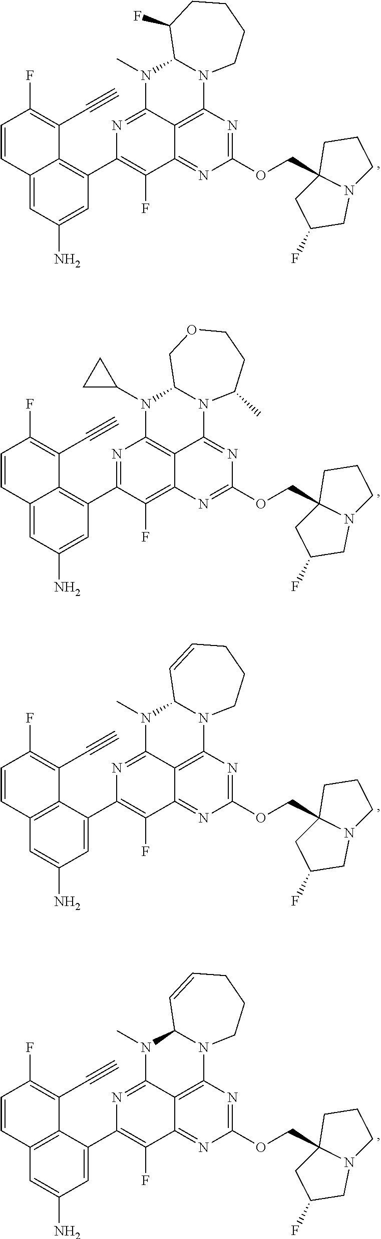 [SMILES: C#Cc1c(F)ccc2cc(N)cc(-c3nc4c5c(nc(OC[C@@]67CCCN6C[C@H](F)C7)nc5c3F)N3CCCC[C@H](F)[C@H]3N4C)c12 ; C#Cc1c(F)ccc2cc(N)cc(-c3nc4c5c(nc(OC[C@@]67CCCN6C[C@H](F)C7)nc5c3F)N3[C@@H](C)CCOC[C@H]3N4C3CC3)c12 ; C#Cc1c(F)ccc2cc(N)cc(-c3nc4c5c(nc(OC[C@@]67CCCN6C[C@H](F)C7)nc5c3F)N3CCCC=C[C@H]3N4C)c12 ; C#Cc1c(F)ccc2cc(N)cc(-c3nc4c5c(nc(OC[C@@]67CCCN6C[C@H](F)C7)nc5c3F)N3CCCC=C[C@@H]3N4C)c12]

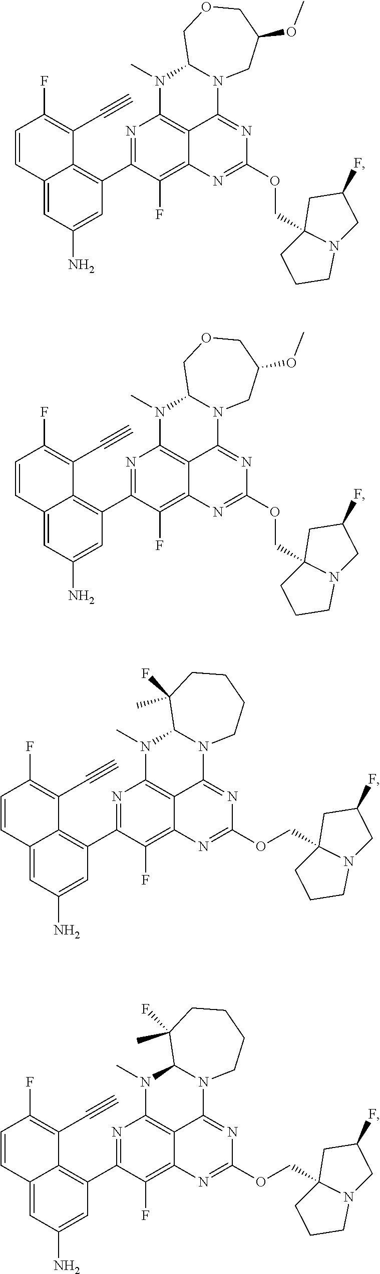 [SMILES: C#Cc1c(F)ccc2cc(N)cc(-c3nc4c5c(nc(OC[C@@]67CCCN6C[C@H](F)C7)nc5c3F)N3C[C@H](OC)COC[C@H]3N4C)c12 ; C#Cc1c(F)ccc2cc(N)cc(-c3nc4c5c(nc(OC[C@@]67CCCN6C[C@H](F)C7)nc5c3F)N3C[C@@H](OC)COC[C@H]3N4C)c12 ; C#Cc1c(F)ccc2cc(N)cc(-c3nc4c5c(nc(OC[C@@]67CCCN6C[C@H](F)C7)nc5c3F)N3CCCC[C@](C)(F)[C@H]3N4C)c12 ; C#Cc1c(F)ccc2cc(N)cc(-c3nc4c5c(nc(OC[C@@]67CCCN6C[C@H](F)C7)nc5c3F)N3CCCC[C@@](C)(F)[C@@H]3N4C)c12]

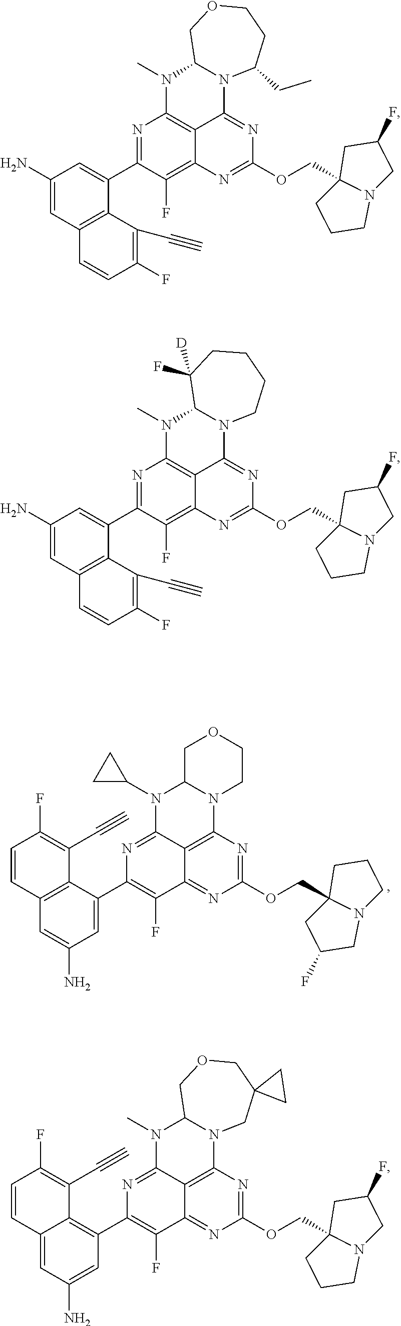 [SMILES: C#Cc1c(F)ccc2cc(N)cc(-c3nc4c5c(nc(OC[C@@]67CCCN6C[C@H](F)C7)nc5c3F)N3[C@@H](CC)CCOC[C@H]3N4C)c12 ; [2H][C@]1(F)CCCCN2c3nc(OC[C@@]45CCCN4C[C@H](F)C5)nc4c(F)c(-c5cc(N)cc6ccc(F)c(C#C)c56)nc(c34)N(C)[C@@H]21 ; C#Cc1c(F)ccc2cc(N)cc(-c3nc4c5c(nc(OC[C@@]67CCCN6C[C@H](F)C7)nc5c3F)N3CCOCC3N4C3CC3)c12 ; C#Cc1c(F)ccc2cc(N)cc(-c3nc4c5c(nc(OC[C@@]67CCCN6C[C@H](F)C7)nc5c3F)N3CC5(CC5)COCC3N4C)c12]

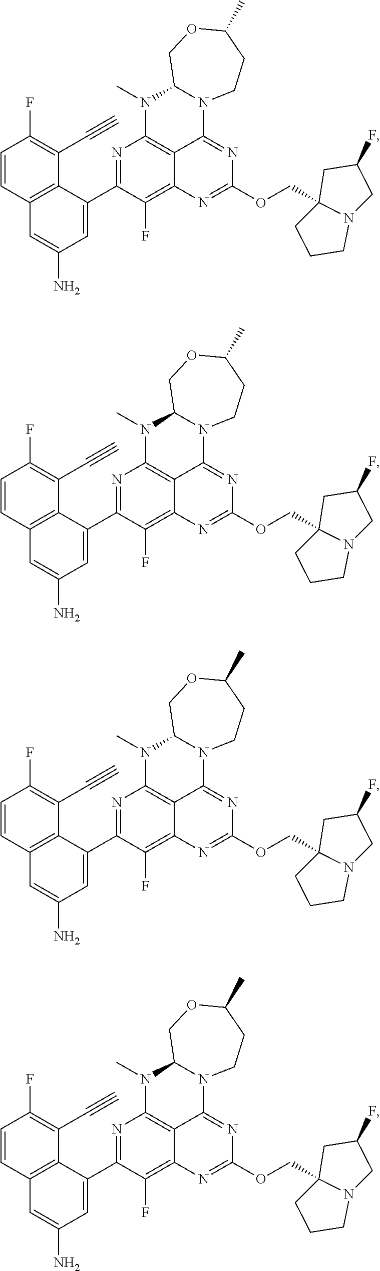 [SMILES: C#Cc1c(F)ccc2cc(N)cc(-c3nc4c5c(nc(OC[C@@]67CCCN6C[C@H](F)C7)nc5c3F)N3CC[C@@H](C)OC[C@H]3N4C)c12 ; C#Cc1c(F)ccc2cc(N)cc(-c3nc4c5c(nc(OC[C@@]67CCCN6C[C@H](F)C7)nc5c3F)N3CC[C@@H](C)OC[C@@H]3N4C)c12 ; C#Cc1c(F)ccc2cc(N)cc(-c3nc4c5c(nc(OC[C@@]67CCCN6C[C@H](F)C7)nc5c3F)N3CC[C@H](C)OC[C@H]3N4C)c12 ; C#Cc1c(F)ccc2cc(N)cc(-c3nc4c5c(nc(OC[C@@]67CCCN6C[C@H](F)C7)nc5c3F)N3CC[C@H](C)OC[C@@H]3N4C)c12]

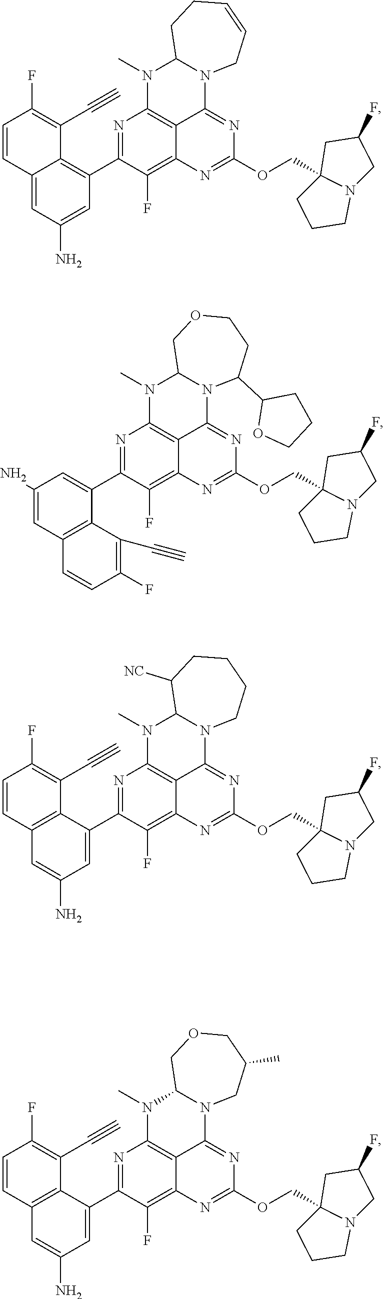 [SMILES: C#Cc1c(F)ccc2cc(N)cc(-c3nc4c5c(nc(OC[C@@]67CCCN6C[C@H](F)C7)nc5c3F)N3CC=CCCC3N4C)c12 ; C#Cc1c(F)ccc2cc(N)cc(-c3nc4c5c(nc(OC[C@@]67CCCN6C[C@H](F)C7)nc5c3F)N3C(C5CCCO5)CCOCC3N4C)c12 ; C#Cc1c(F)ccc2cc(N)cc(-c3nc4c5c(nc(OC[C@@]67CCCN6C[C@H](F)C7)nc5c3F)N3CCCCC(C#N)C3N4C)c12 ; C#Cc1c(F)ccc2cc(N)cc(-c3nc4c5c(nc(OC[C@@]67CCCN6C[C@H](F)C7)nc5c3F)N3C[C@@H](C)COC[C@H]3N4C)c12]

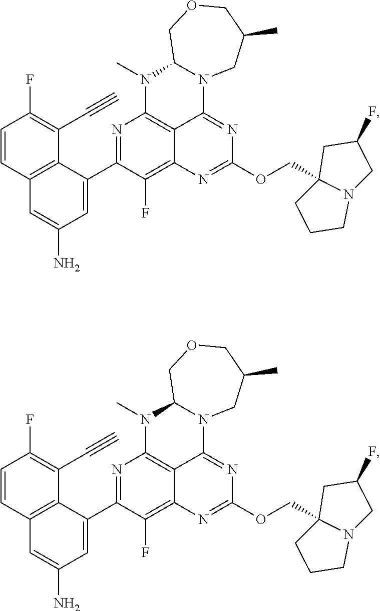 [SMILES: C#Cc1c(F)ccc2cc(N)cc(-c3nc4c5c(nc(OC[C@@]67CCCN6C[C@H](F)C7)nc5c3F)N3C[C@H](C)COC[C@H]3N4C)c12 ; C#Cc1c(F)ccc2cc(N)cc(-c3nc4c5c(nc(OC[C@@]67CCCN6C[C@H](F)C7)nc5c3F)N3C[C@H](C)COC[C@@H]3N4C)c12]

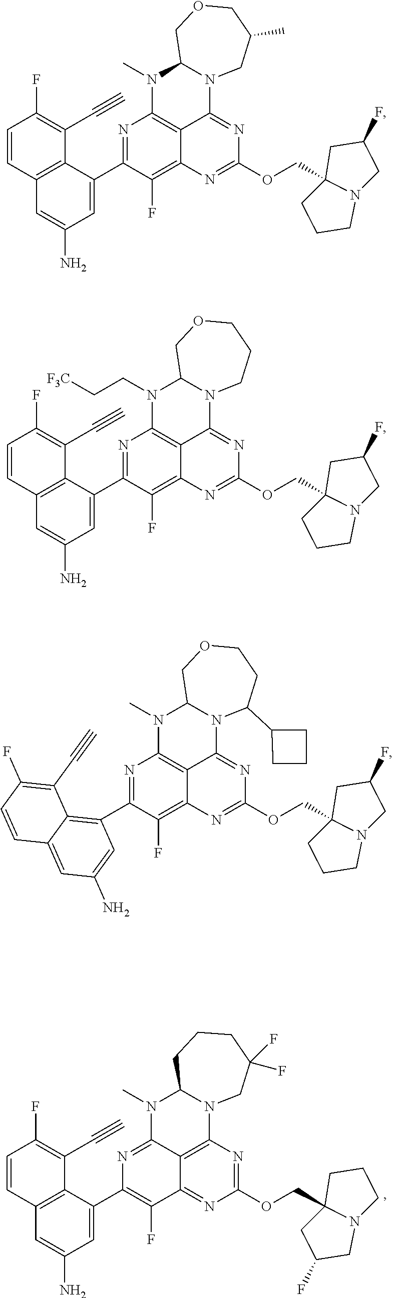 [SMILES: C#Cc1c(F)ccc2cc(N)cc(-c3nc4c5c(nc(OC[C@@]67CCCN6C[C@H](F)C7)nc5c3F)N3C[C@@H](C)COC[C@@H]3N4C)c12 ; C#Cc1c(F)ccc2cc(N)cc(-c3nc4c5c(nc(OC[C@@]67CCCN6C[C@H](F)C7)nc5c3F)N3CCCOCC3N4CCC(F)(F)F)c12 ; C#Cc1c(F)ccc2cc(N)cc(-c3nc4c5c(nc(OC[C@@]67CCCN6C[C@H](F)C7)nc5c3F)N3C(C5CCC5)CCOCC3N4C)c12 ; C#Cc1c(F)ccc2cc(N)cc(-c3nc4c5c(nc(OC[C@@]67CCCN6C[C@H](F)C7)nc5c3F)N3CC(F)(F)CCC[C@@H]3N4C)c12]

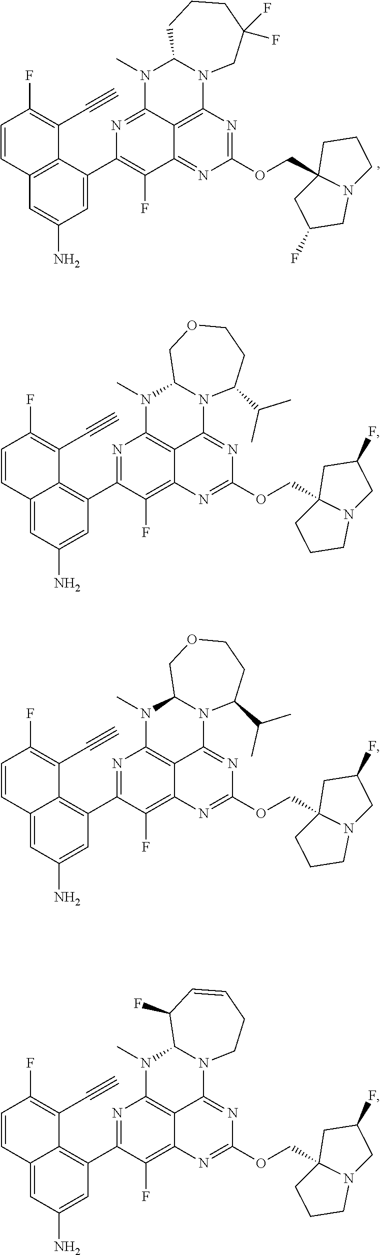 [SMILES: C#Cc1c(F)ccc2cc(N)cc(-c3nc4c5c(nc(OC[C@@]67CCCN6C[C@H](F)C7)nc5c3F)N3CC(F)(F)CCC[C@H]3N4C)c12 ; C#Cc1c(F)ccc2cc(N)cc(-c3nc4c5c(nc(OC[C@@]67CCCN6C[C@H](F)C7)nc5c3F)N3[C@@H](C(C)C)CCOC[C@H]3N4C)c12 ; C#Cc1c(F)ccc2cc(N)cc(-c3nc4c5c(nc(OC[C@@]67CCCN6C[C@H](F)C7)nc5c3F)N3[C@H](COCC[C@H]3C(C)C)N4C)c12 ; C#Cc1c(F)ccc2cc(N)cc(-c3nc4c5c(nc(OC[C@@]67CCCN6C[C@H](F)C7)nc5c3F)N3CCC=C[C@H](F)[C@H]3N4C)c12]

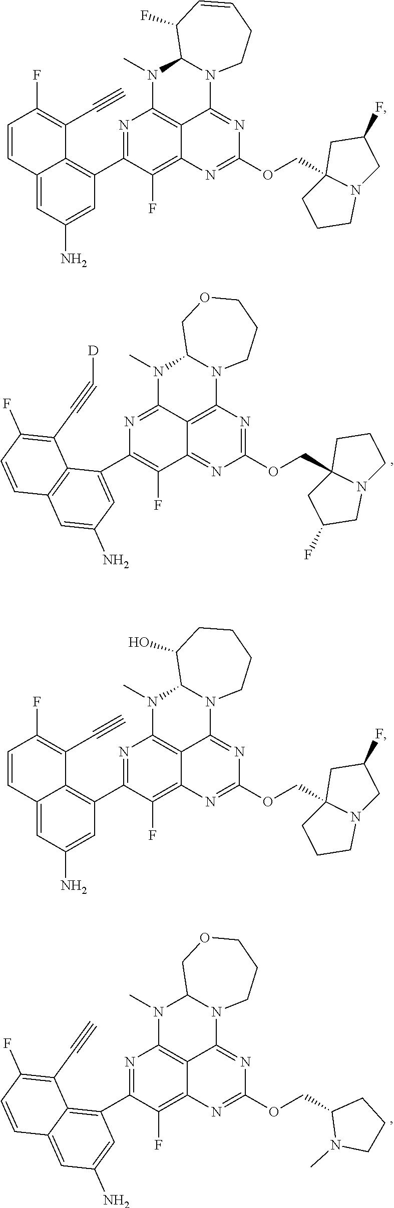 [SMILES: C#Cc1c(F)ccc2cc(N)cc(-c3nc4c5c(nc(OC[C@@]67CCCN6C[C@H](F)C7)nc5c3F)N3CCC=C[C@@H](F)[C@@H]3N4C)c12 ; [2H]C#Cc1c(F)ccc2cc(N)cc(-c3nc4c5c(nc(OC[C@@]67CCCN6C[C@H](F)C7)nc5c3F)N3CCCOC[C@H]3N4C)c12 ; C#Cc1c(F)ccc2cc(N)cc(-c3nc4c5c(nc(OC[C@@]67CCCN6C[C@H](F)C7)nc5c3F)N3CCCC[C@@H](O)[C@H]3N4C)c12 ; C#Cc1c(F)ccc2cc(N)cc(-c3nc4c5c(nc(OC[C@@H]6CCCN6C)nc5c3F)N3CCCOCC3N4C)c12]

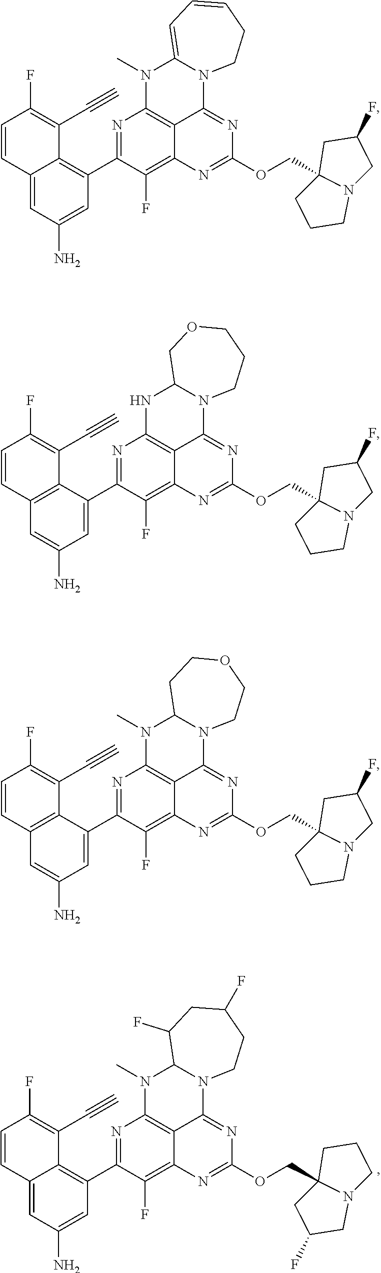 [SMILES: C#Cc1c(F)ccc2cc(N)cc(-c3nc4c5c(nc(OC[C@@]67CCCN6C[C@H](F)C7)nc5n5c(n4C)=CC=CCC5)c3F)c12 ; C#Cc1c(F)ccc2cc(N)cc(-c3nc4c5c(nc(OC[C@@]67CCCN6C[C@H](F)C7)nc5c3F)N3CCCOCC3N4)c12 ; C#Cc1c(F)ccc2cc(N)cc(-c3nc4c5c(nc(OC[C@@]67CCCN6C[C@H](F)C7)nc5c3F)N3CCOCCC3N4C)c12 ; C#Cc1c(F)ccc2cc(N)cc(-c3nc4c5c(nc(OC[C@@]67CCCN6C[C@H](F)C7)nc5c3F)N3CCC(F)CC(F)C3N4C)c12]

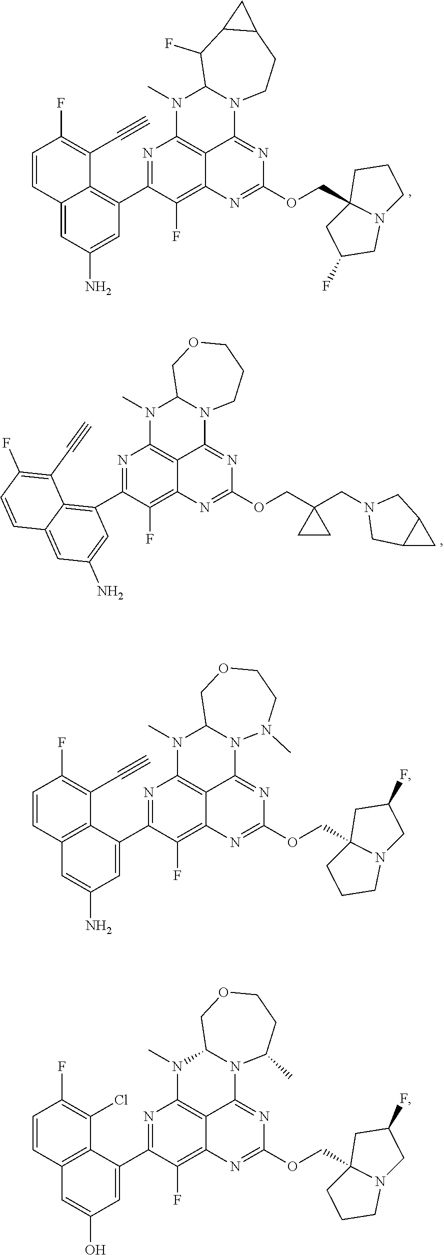 [SMILES: C#Cc1c(F)ccc2cc(N)cc(-c3nc4c5c(nc(OC[C@@]67CCCN6C[C@H](F)C7)nc5c3F)N3CCC5CC5C(F)C3N4C)c12 ; C#Cc1c(F)ccc2cc(N)cc(-c3nc4c5c(nc(OCC6(CN7CC8CC8C7)CC6)nc5c3F)N3CCCOCC3N4C)c12 ; C#Cc1c(F)ccc2cc(N)cc(-c3nc4c5c(nc(OC[C@@]67CCCN6C[C@H](F)C7)nc5c3F)N3C(COCCN3C)N4C)c12 ; C[C@H]1CCOC[C@H]2N(C)c3nc(-c4cc(O)cc5ccc(F)c(Cl)c45)c(F)c4nc(OC[C@@]56CCCN5C[C@H](F)C6)nc(c34)N12]

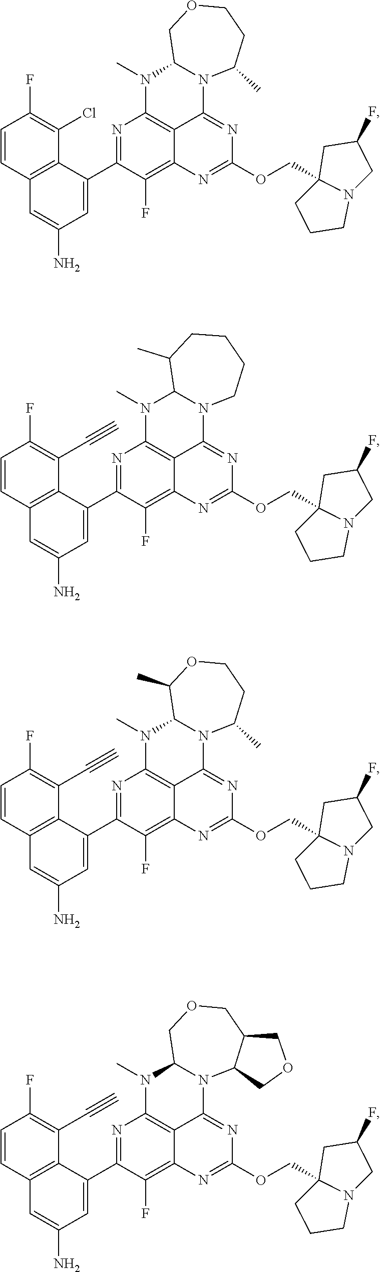 [SMILES: C[C@H]1CCOC[C@H]2N(C)c3nc(-c4cc(N)cc5ccc(F)c(Cl)c45)c(F)c4nc(OC[C@@]56CCCN5C[C@H](F)C6)nc(c34)N12 ; C#Cc1c(F)ccc2cc(N)cc(-c3nc4c5c(nc(OC[C@@]67CCCN6C[C@H](F)C7)nc5c3F)N3CCCCC(C)C3N4C)c12 ; C#Cc1c(F)ccc2cc(N)cc(-c3nc4c5c(nc(OC[C@@]67CCCN6C[C@H](F)C7)nc5c3F)N3[C@@H](C)CCO[C@H](C)[C@H]3N4C)c12 ; C#Cc1c(F)ccc2cc(N)cc(-c3nc4c5c(nc(OC[C@@]67CCCN6C[C@H](F)C7)nc5c3F)N3[C@@H]5COC[C@@H]5COC[C@@H]3N4C)c12]

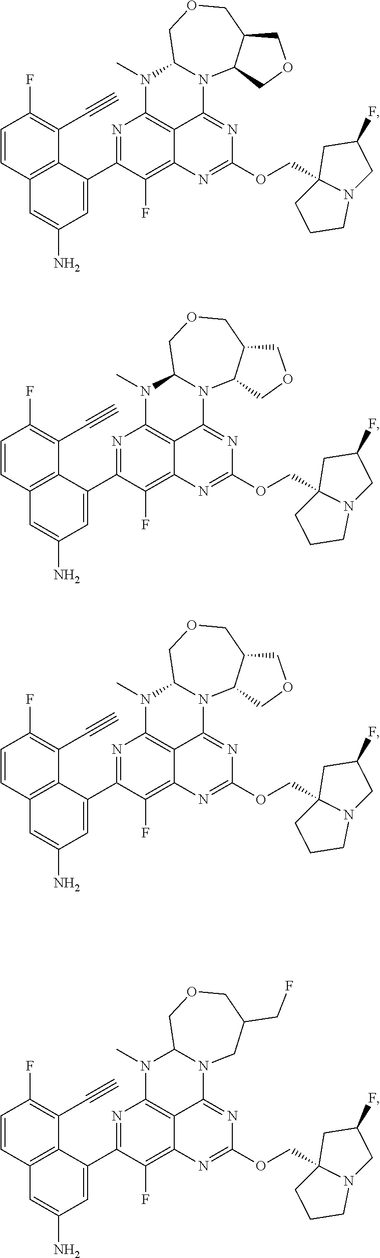 [SMILES: C#Cc1c(F)ccc2cc(N)cc(-c3nc4c5c(nc(OC[C@@]67CCCN6C[C@H](F)C7)nc5c3F)N3[C@@H]5COC[C@@H]5COC[C@H]3N4C)c12 ; C#Cc1c(F)ccc2cc(N)cc(-c3nc4c5c(nc(OC[C@@]67CCCN6C[C@H](F)C7)nc5c3F)N3[C@H](COC[C@@H]5COC[C@@H]53)N4C)c12 ; C#Cc1c(F)ccc2cc(N)cc(-c3nc4c5c(nc(OC[C@@]67CCCN6C[C@H](F)C7)nc5c3F)N3[C@H]5COC[C@H]5COC[C@H]3N4C)c12 ; C#Cc1c(F)ccc2cc(N)cc(-c3nc4c5c(nc(OC[C@@]67CCCN6C[C@H](F)C7)nc5c3F)N3CC(CF)COCC3N4C)c12]

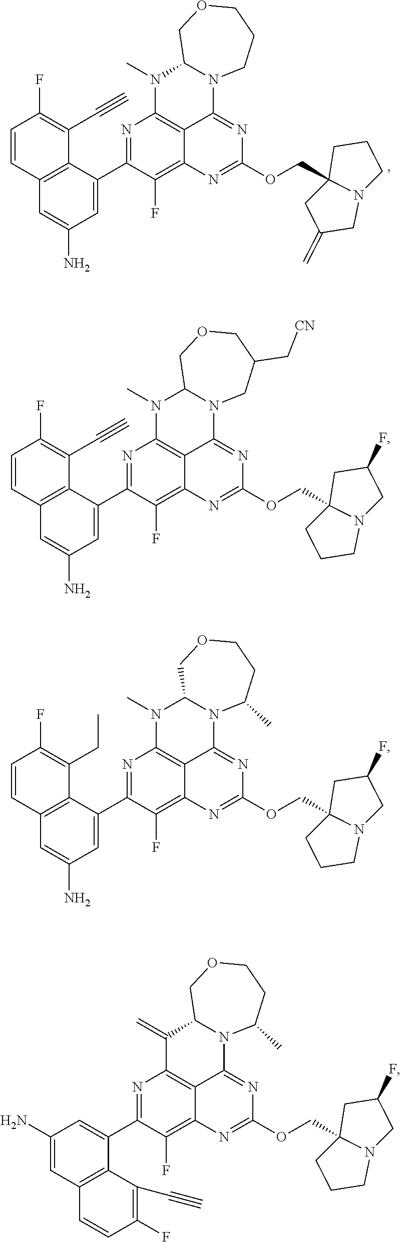 [SMILES: C#Cc1c(F)ccc2cc(N)cc(-c3nc4c5c(nc(OC[C@@]67CCCN6CC(=C)C7)nc5c3F)N3CCCOC[C@H]3N4C)c12 ; C#Cc1c(F)ccc2cc(N)cc(-c3nc4c5c(nc(OC[C@@]67CCCN6C[C@H](F)C7)nc5c3F)N3CC(CC#N)COCC3N4C)c12 ; CCc1c(F)ccc2cc(N)cc(-c3nc4c5c(nc(OC[C@@]67CCCN6C[C@H](F)C7)nc5c3F)N3[C@@H](C)CCOC[C@H]3N4C)c12 ; C#Cc1c(F)ccc2cc(N)cc(-c3nc4c5c(nc(OC[C@@]67CCCN6C[C@H](F)C7)nc5c3F)N3[C@@H](C)CCOC[C@H]3C4=C)c12]

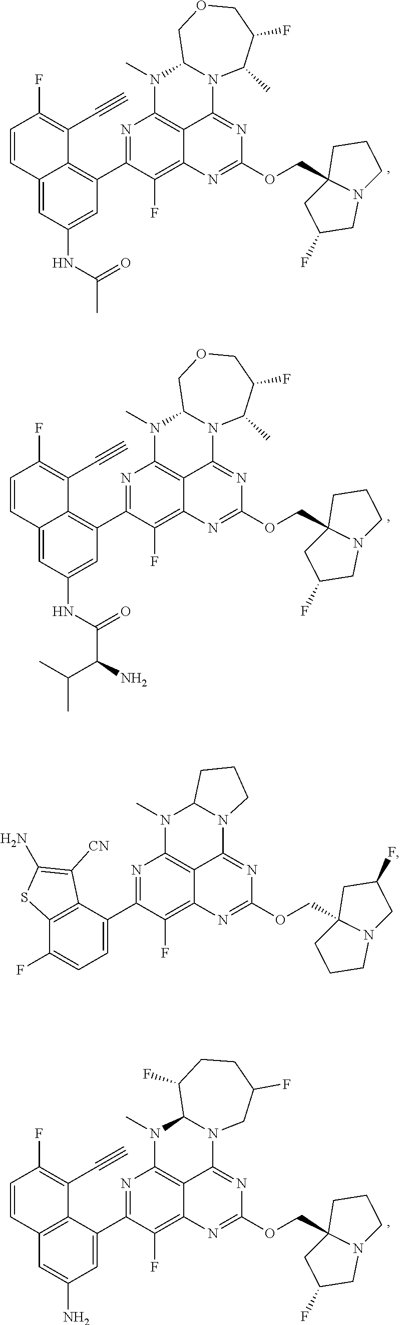 [SMILES: C#Cc1c(F)ccc2cc(NC(C)=O)cc(-c3nc4c5c(nc(OC[C@@]67CCCN6C[C@H](F)C7)nc5c3F)N3[C@@H](C)[C@@H](F)COC[C@H]3N4C)c12 ; C#Cc1c(F)ccc2cc(NC(=O)[C@@H](N)C(C)C)cc(-c3nc4c5c(nc(OC[C@@]67CCCN6C[C@H](F)C7)nc5c3F)N3[C@@H](C)[C@@H](F)COC[C@H]3N4C)c12 ; CN1c2nc(-c3ccc(F)c4sc(N)c(C#N)c34)c(F)c3nc(OC[C@@]45CCCN4C[C@H](F)C5)nc(c23)N2CCCC12 ; C#Cc1c(F)ccc2cc(N)cc(-c3nc4c5c(nc(OC[C@@]67CCCN6C[C@H](F)C7)nc5c3F)N3CC(F)CC[C@@H](F)[C@@H]3N4C)c12]

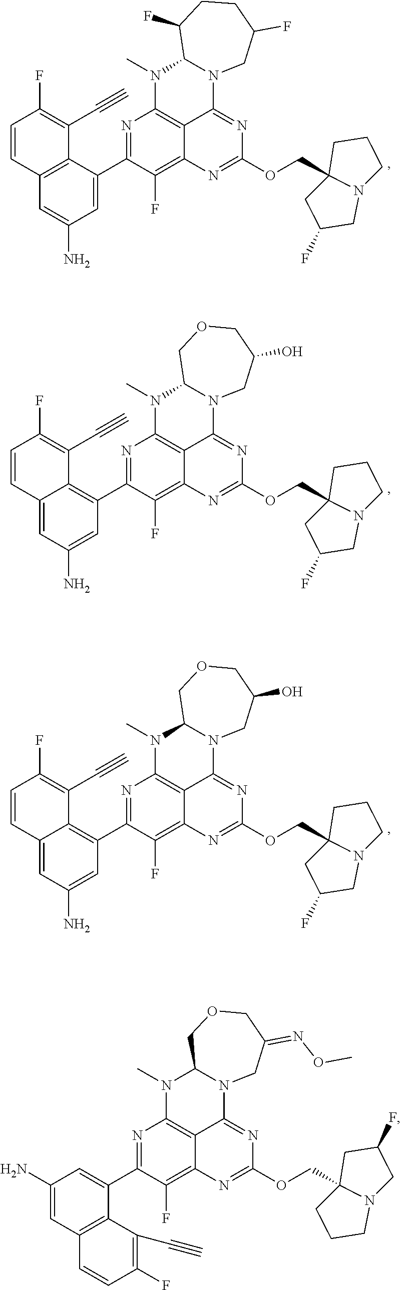 [SMILES: C#Cc1c(F)ccc2cc(N)cc(-c3nc4c5c(nc(OC[C@@]67CCCN6C[C@H](F)C7)nc5c3F)N3CC(F)CC[C@H](F)[C@H]3N4C)c12 ; C#Cc1c(F)ccc2cc(N)cc(-c3nc4c5c(nc(OC[C@@]67CCCN6C[C@H](F)C7)nc5c3F)N3C[C@@H](O)COC[C@H]3N4C)c12 ; C#Cc1c(F)ccc2cc(N)cc(-c3nc4c5c(nc(OC[C@@]67CCCN6C[C@H](F)C7)nc5c3F)N3C[C@H](O)COC[C@@H]3N4C)c12 ; C#Cc1c(F)ccc2cc(N)cc(-c3nc4c5c(nc(OC[C@@]67CCCN6C[C@H](F)C7)nc5c3F)N3C/C(=N\OC)COC[C@@H]3N4C)c12]

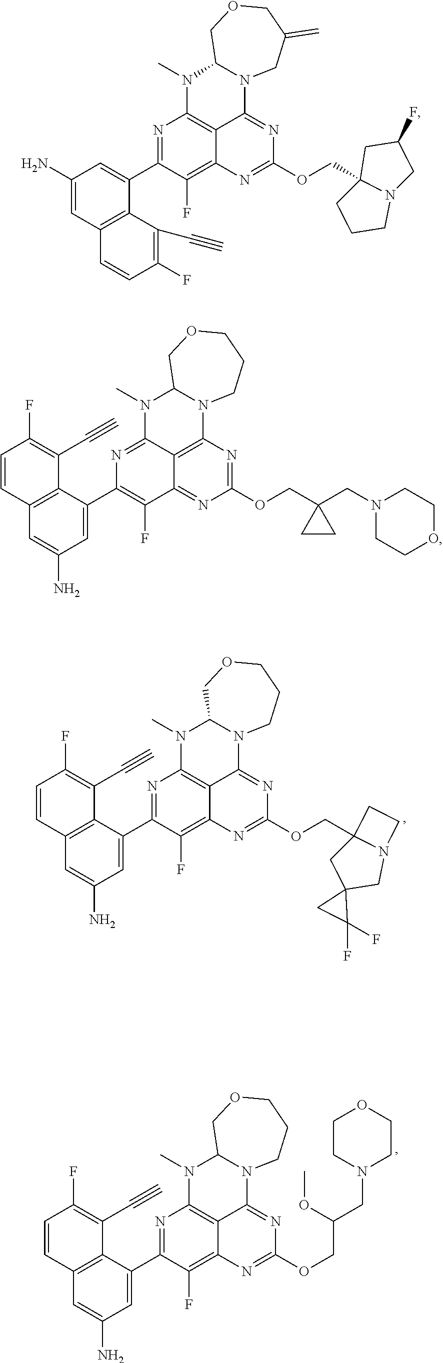 [SMILES: C#Cc1c(F)ccc2cc(N)cc(-c3nc4c5c(nc(OC[C@@]67CCCN6C[C@H](F)C7)nc5c3F)N3CC(=C)COC[C@H]3N4C)c12 ; C#Cc1c(F)ccc2cc(N)cc(-c3nc4c5c(nc(OCC6(CN7CCOCC7)CC6)nc5c3F)N3CCCOCC3N4C)c12 ; C#Cc1c(F)ccc2cc(N)cc(-c3nc4c5c(nc(OCC67CCN6CC6(C7)CC6(F)F)nc5c3F)N3CCCOC[C@H]3N4C)c12 ; C#Cc1c(F)ccc2cc(N)cc(-c3nc4c5c(nc(OCC(CN6CCOCC6)OC)nc5c3F)N3CCCOCC3N4C)c12]

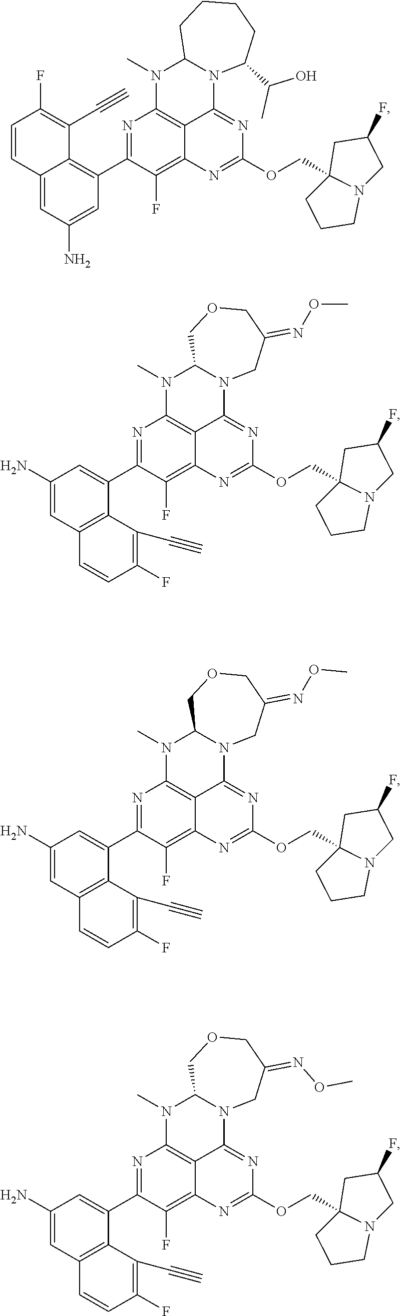 [SMILES: C#Cc1c(F)ccc2cc(N)cc(-c3nc4c5c(nc(OC[C@@]67CCCN6C[C@H](F)C7)nc5c3F)N3C(CCCC[C@@H]3C(C)O)N4C)c12 ; C#Cc1c(F)ccc2cc(N)cc(-c3nc4c5c(nc(OC[C@@]67CCCN6C[C@H](F)C7)nc5c3F)N3C/C(=N/OC)COC[C@H]3N4C)c12 ; C#Cc1c(F)ccc2cc(N)cc(-c3nc4c5c(nc(OC[C@@]67CCCN6C[C@H](F)C7)nc5c3F)N3C/C(=N/OC)COC[C@@H]3N4C)c12 ; C#Cc1c(F)ccc2cc(N)cc(-c3nc4c5c(nc(OC[C@@]67CCCN6C[C@H](F)C7)nc5c3F)N3C/C(=N\OC)COC[C@H]3N4C)c12]

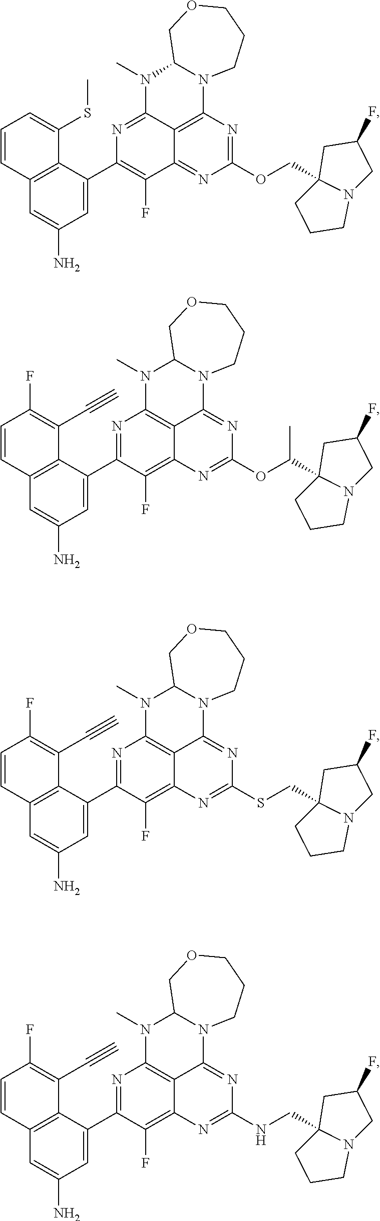 [SMILES: CSc1cccc2cc(N)cc(-c3nc4c5c(nc(OC[C@@]67CCCN6C[C@H](F)C7)nc5c3F)N3CCCOC[C@H]3N4C)c12 ; C#Cc1c(F)ccc2cc(N)cc(-c3nc4c5c(nc(OC(C)[C@@]67CCCN6C[C@H](F)C7)nc5c3F)N3CCCOCC3N4C)c12 ; C#Cc1c(F)ccc2cc(N)cc(-c3nc4c5c(nc(SC[C@@]67CCCN6C[C@H](F)C7)nc5c3F)N3CCCOCC3N4C)c12 ; C#Cc1c(F)ccc2cc(N)cc(-c3nc4c5c(nc(NC[C@@]67CCCN6C[C@H](F)C7)nc5c3F)N3CCCOCC3N4C)c12]

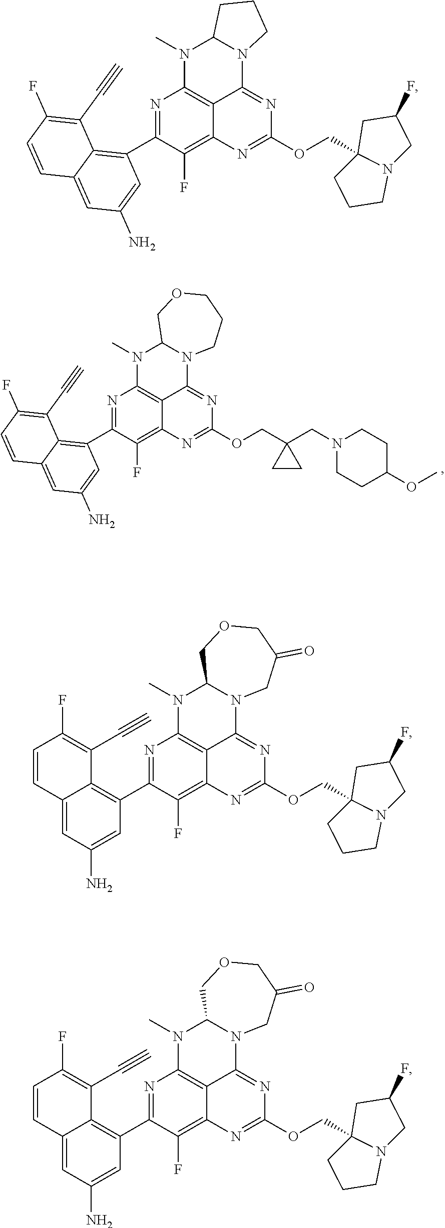 [SMILES: C#Cc1c(F)ccc2cc(N)cc(-c3nc4c5c(nc(OC[C@@]67CCCN6C[C@H](F)C7)nc5c3F)N3CCCC3N4C)c12 ; C#Cc1c(F)ccc2cc(N)cc(-c3nc4c5c(nc(OCC6(CN7CCC(OC)CC7)CC6)nc5c3F)N3CCCOCC3N4C)c12 ; C#Cc1c(F)ccc2cc(N)cc(-c3nc4c5c(nc(OC[C@@]67CCCN6C[C@H](F)C7)nc5c3F)N3CC(=O)COC[C@@H]3N4C)c12 ; C#Cc1c(F)ccc2cc(N)cc(-c3nc4c5c(nc(OC[C@@]67CCCN6C[C@H](F)C7)nc5c3F)N3CC(=O)COC[C@H]3N4C)c12]

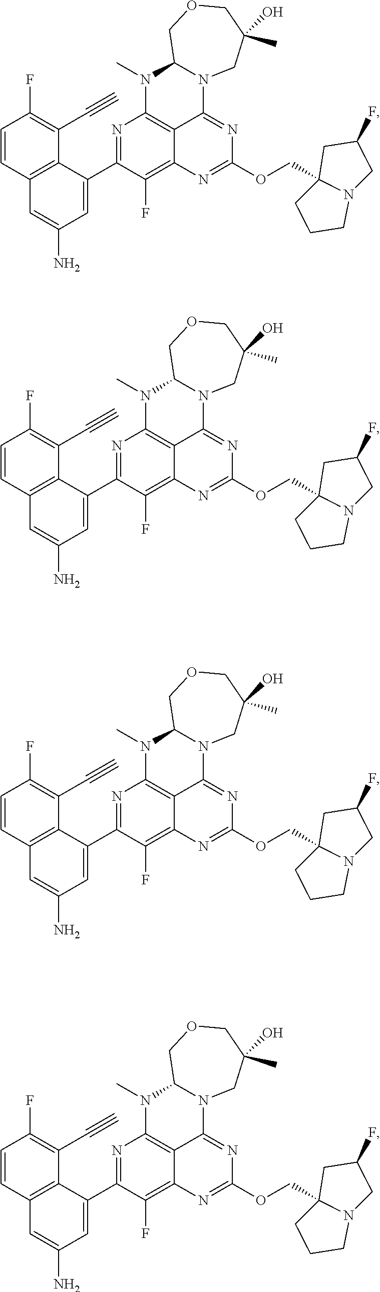 [SMILES: C#Cc1c(F)ccc2cc(N)cc(-c3nc4c5c(nc(OC[C@@]67CCCN6C[C@H](F)C7)nc5c3F)N3C[C@@](C)(O)COC[C@@H]3N4C)c12 ; C#Cc1c(F)ccc2cc(N)cc(-c3nc4c5c(nc(OC[C@@]67CCCN6C[C@H](F)C7)nc5c3F)N3C[C@](C)(O)COC[C@H]3N4C)c12 ; C#Cc1c(F)ccc2cc(N)cc(-c3nc4c5c(nc(OC[C@@]67CCCN6C[C@H](F)C7)nc5c3F)N3C[C@](C)(O)COC[C@@H]3N4C)c12 ; C#Cc1c(F)ccc2cc(N)cc(-c3nc4c5c(nc(OC[C@@]67CCCN6C[C@H](F)C7)nc5c3F)N3C[C@@](C)(O)COC[C@H]3N4C)c12]

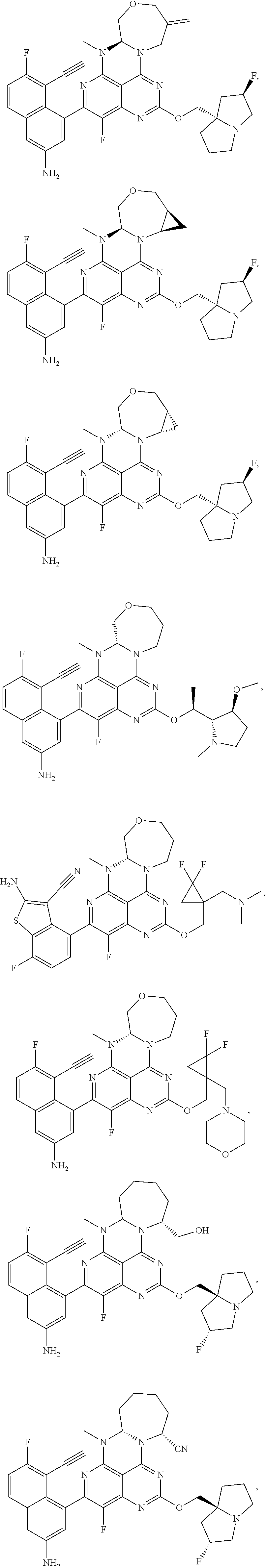

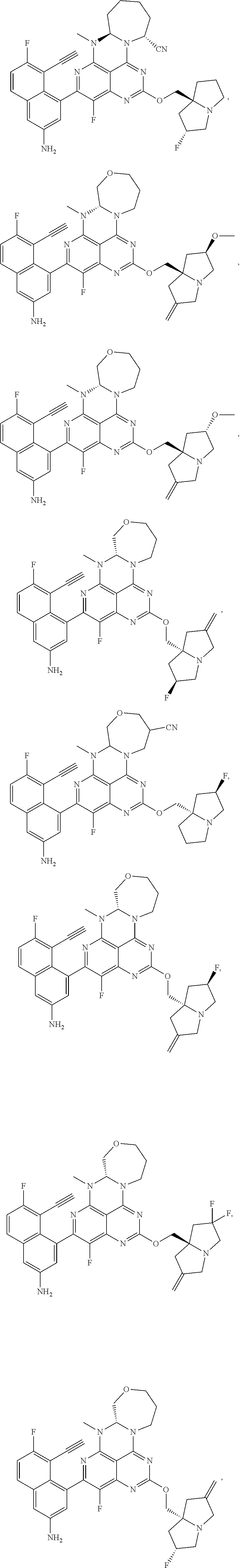

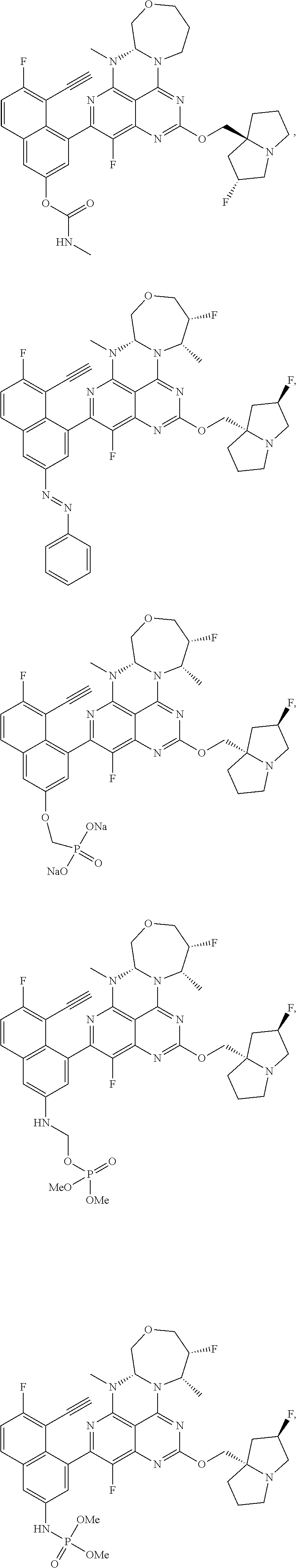

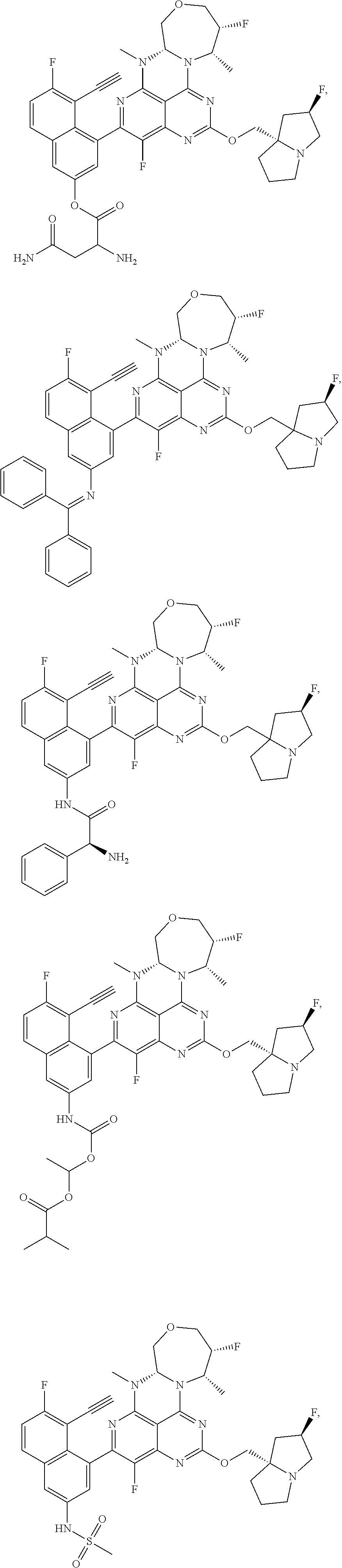

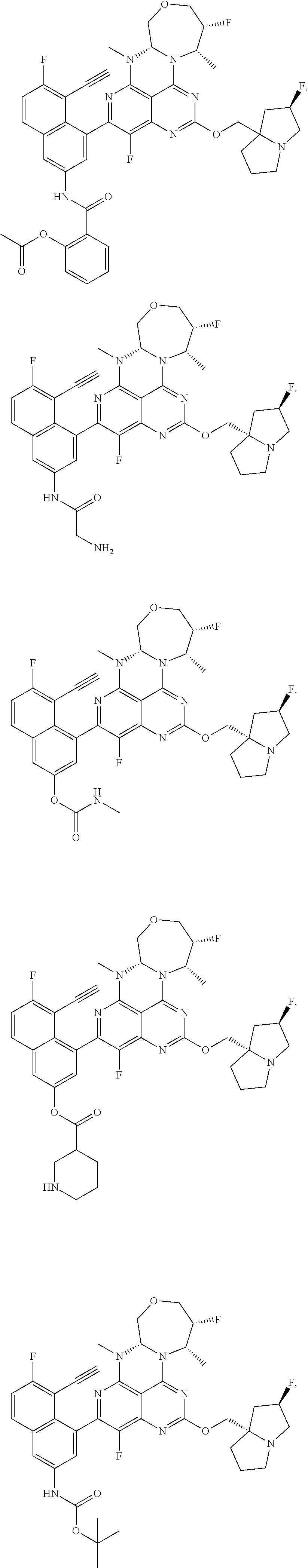

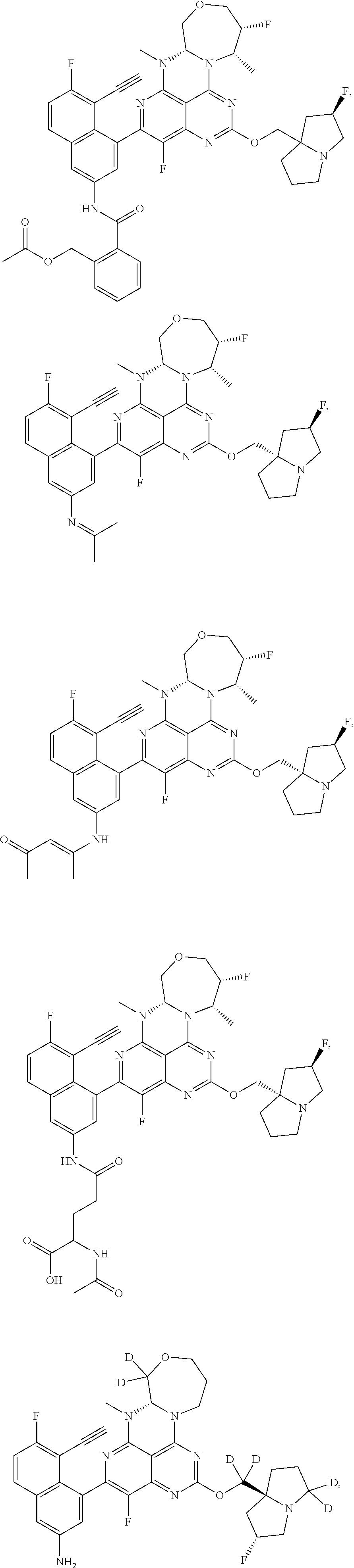

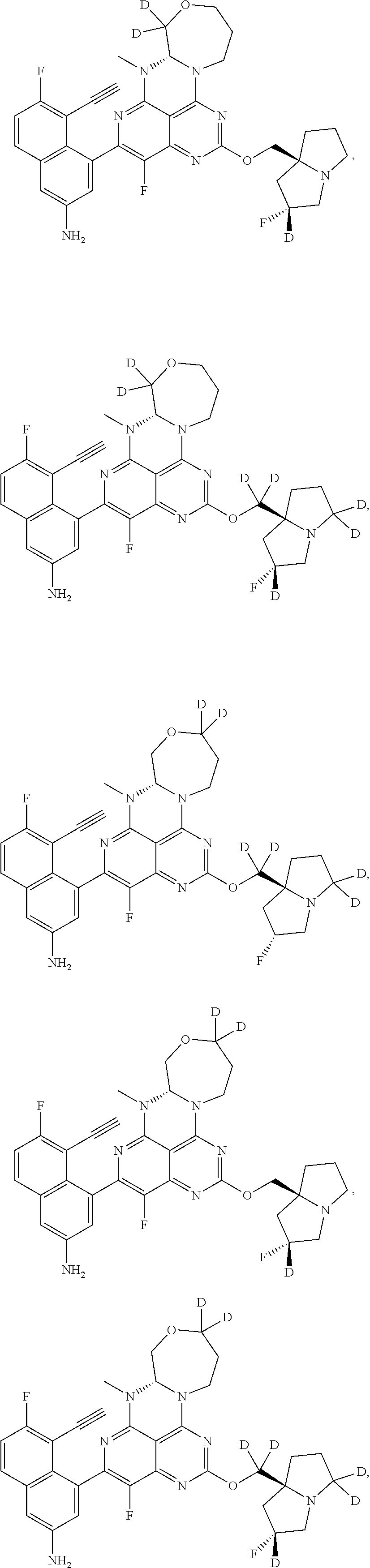

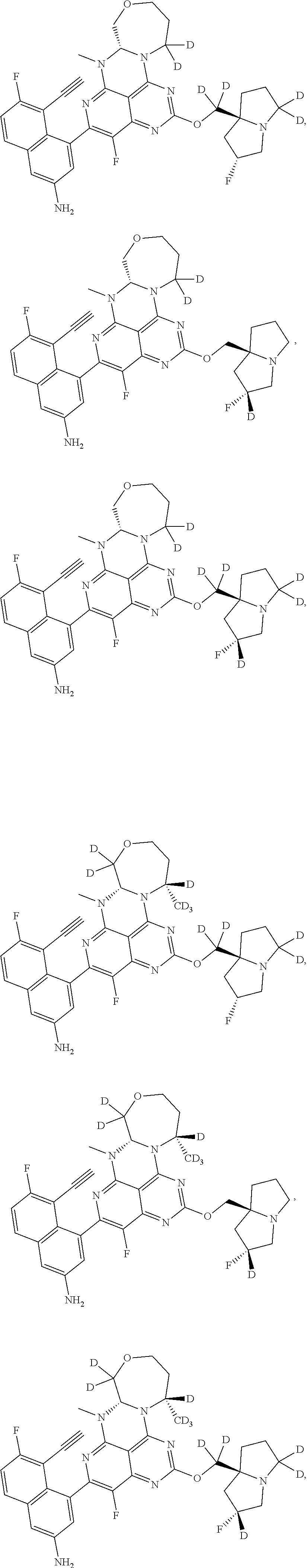

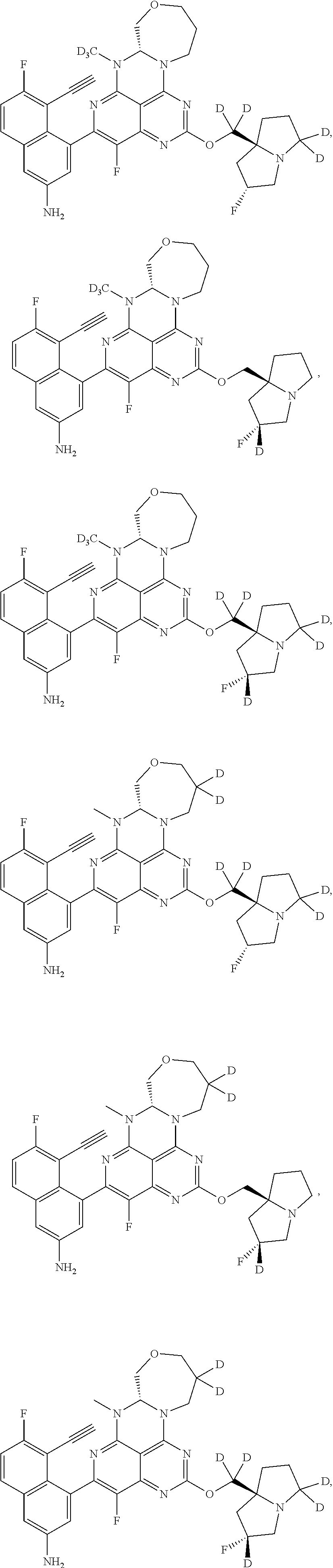

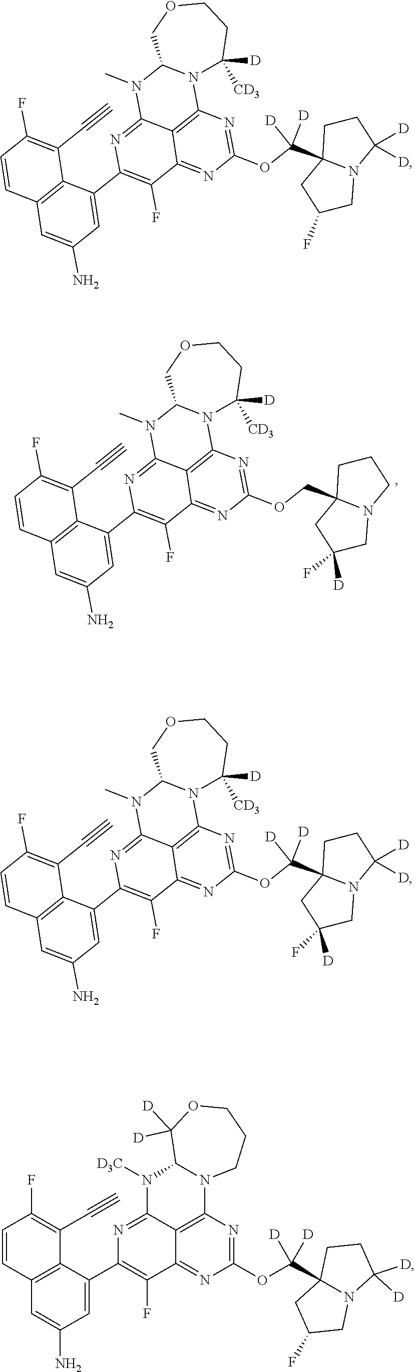

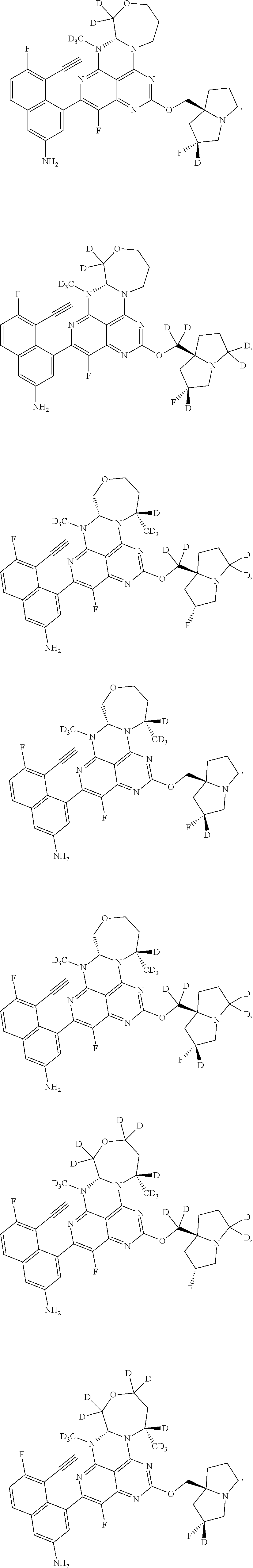

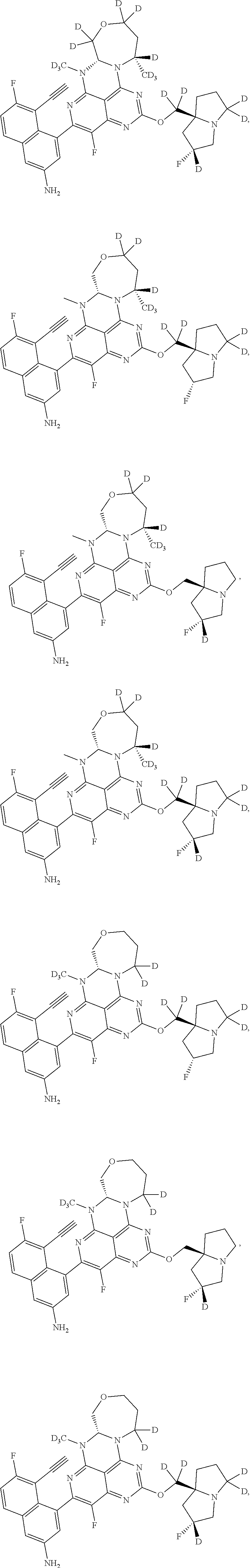

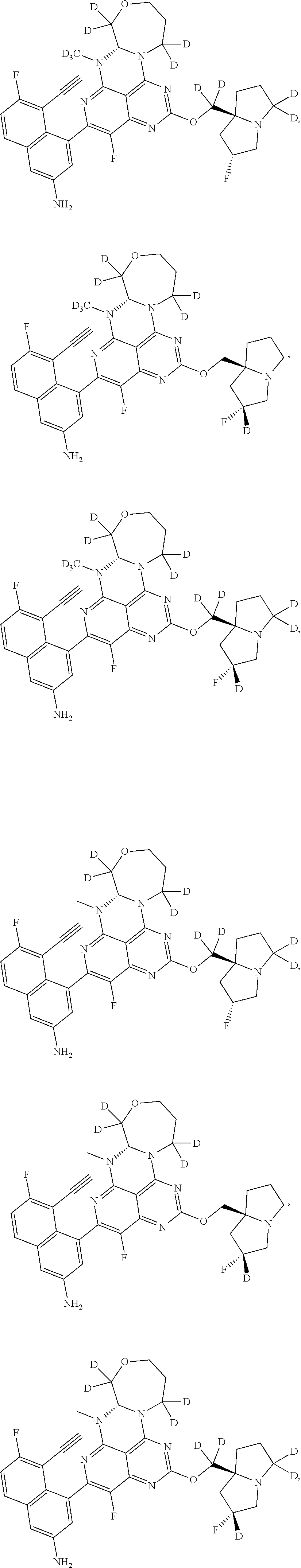

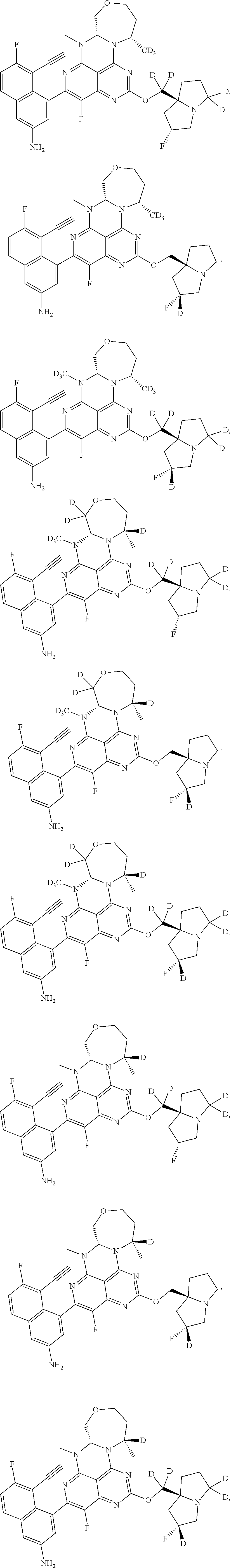

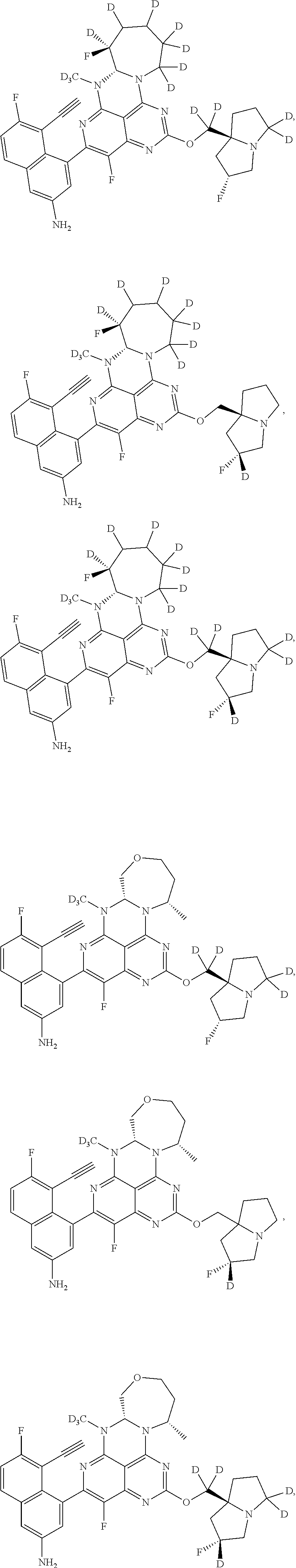

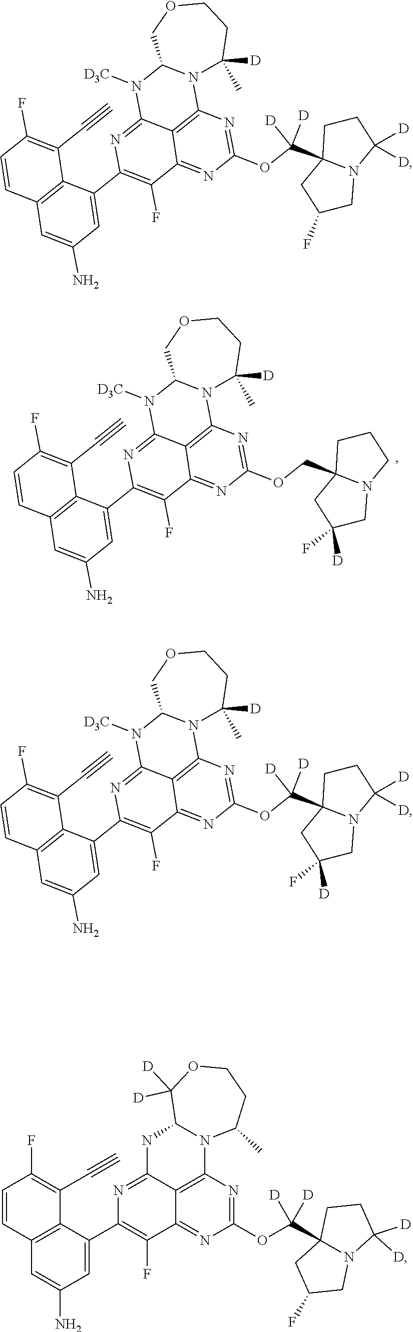

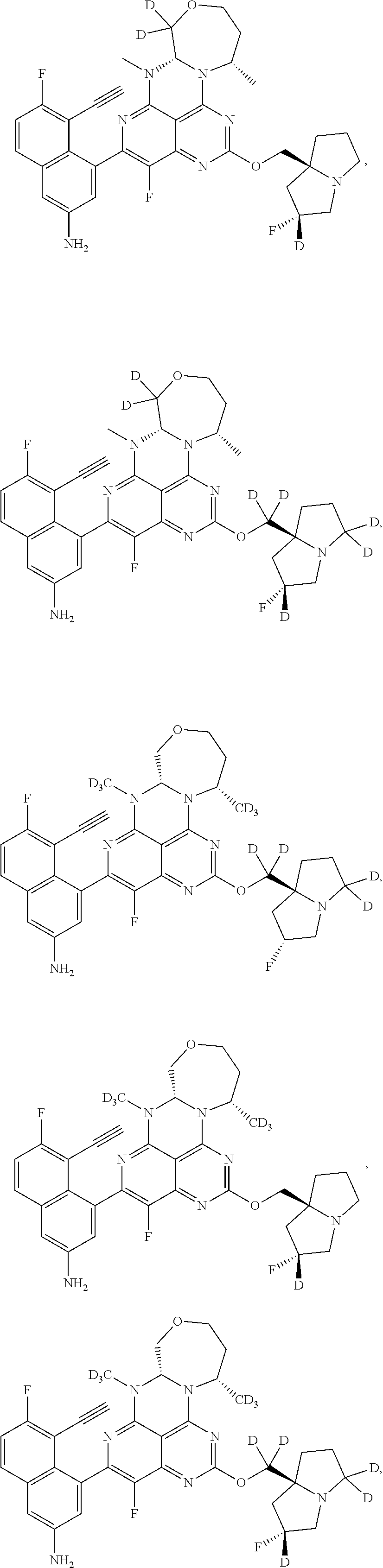

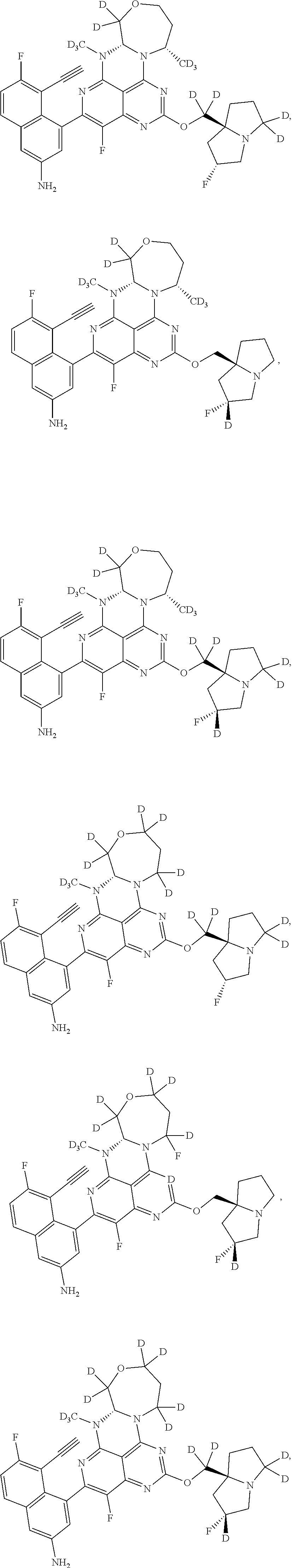

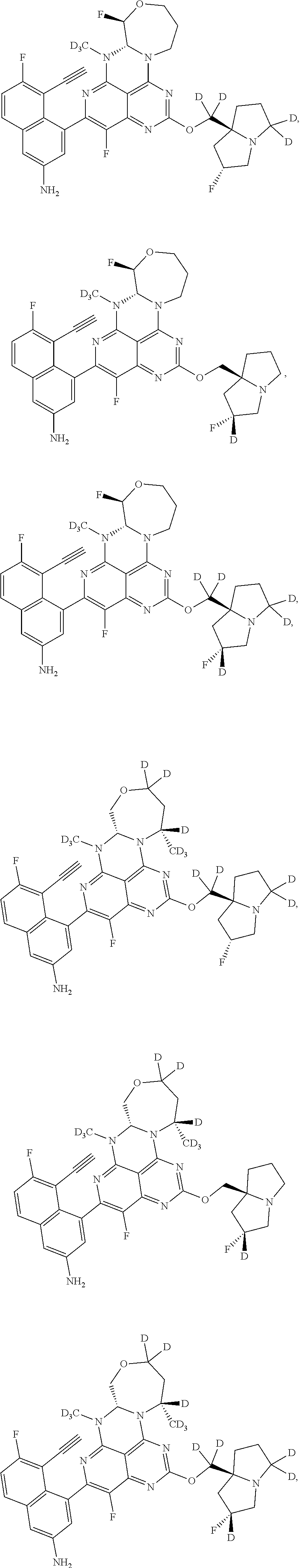

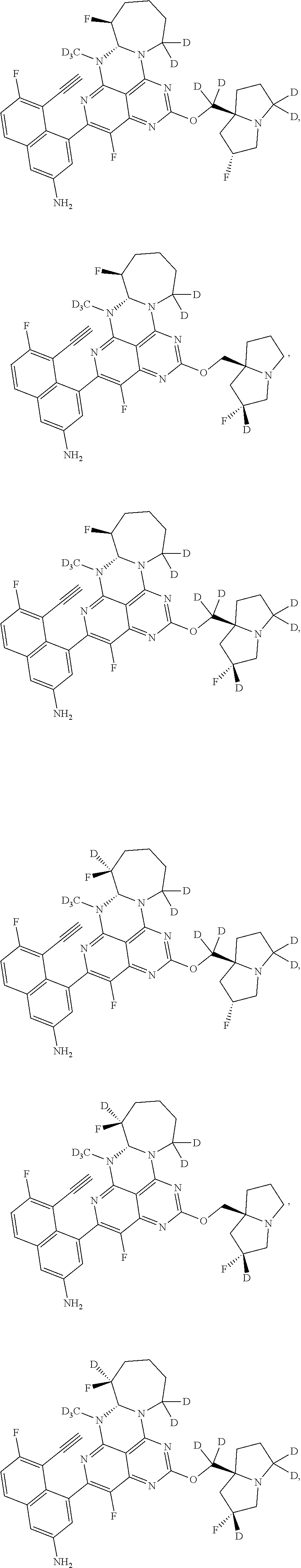

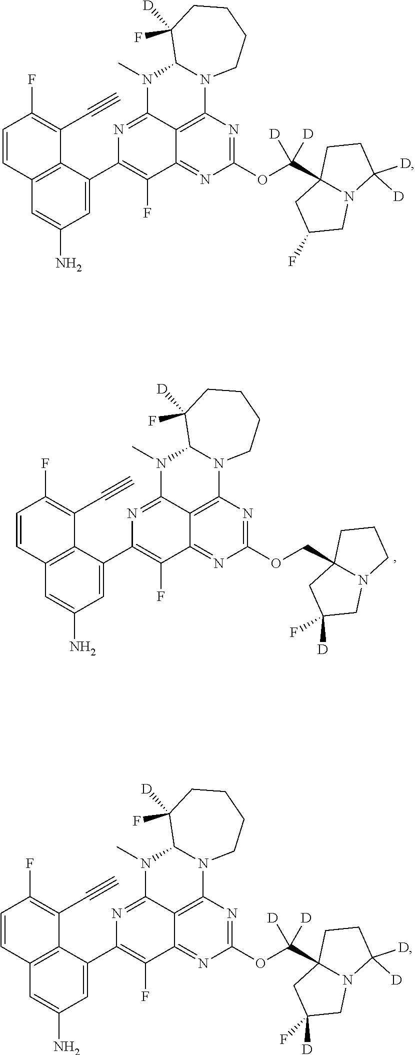

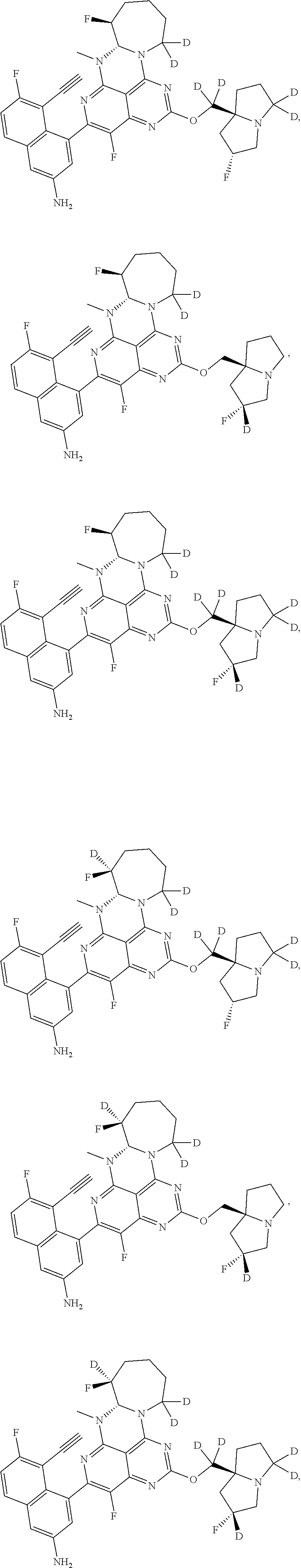

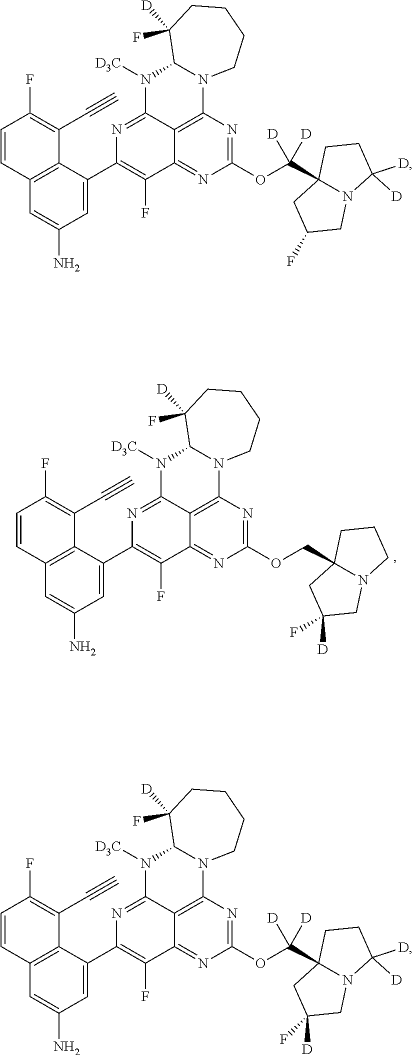

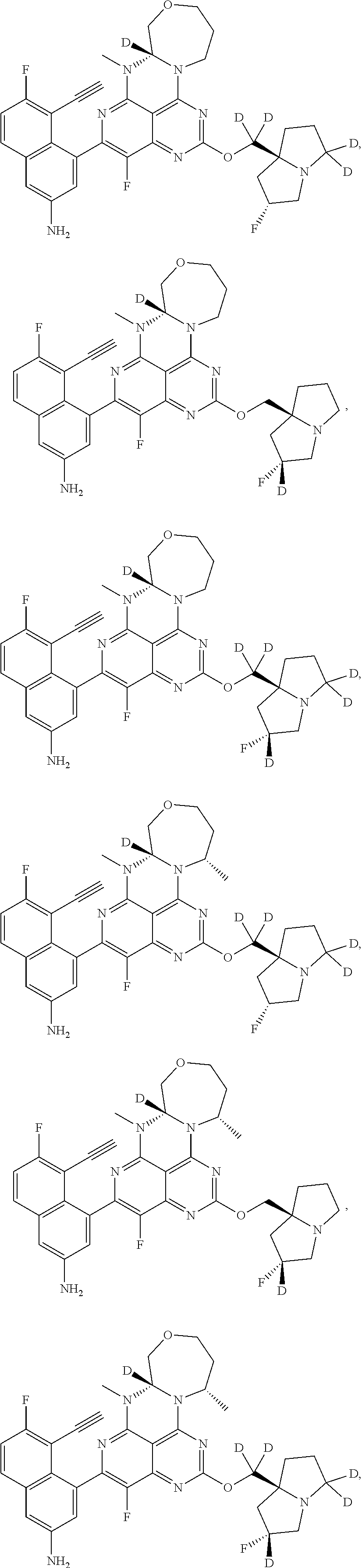

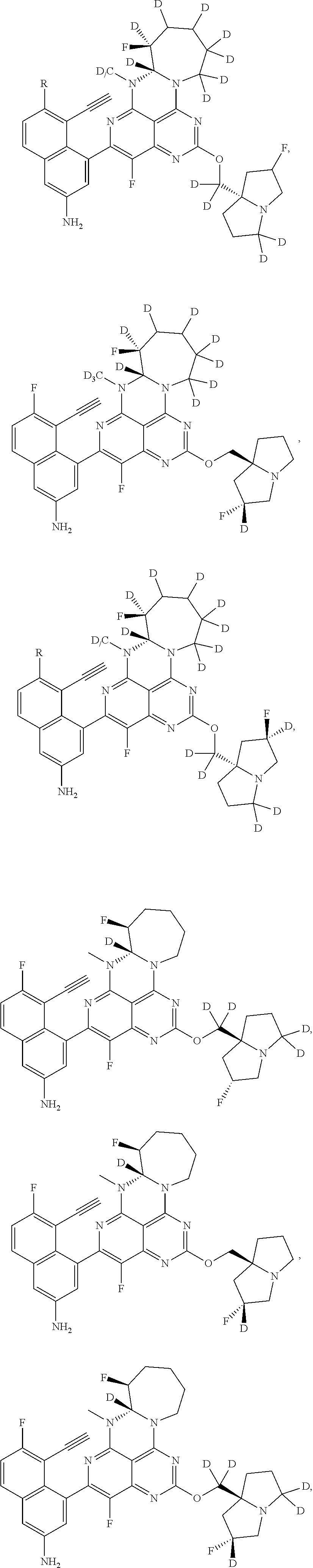

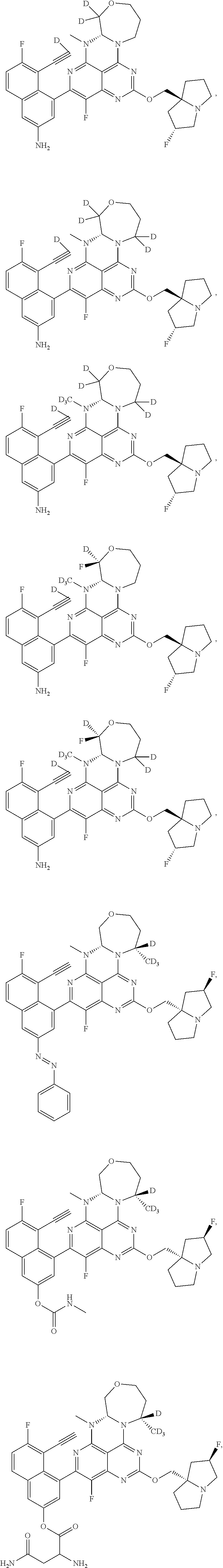

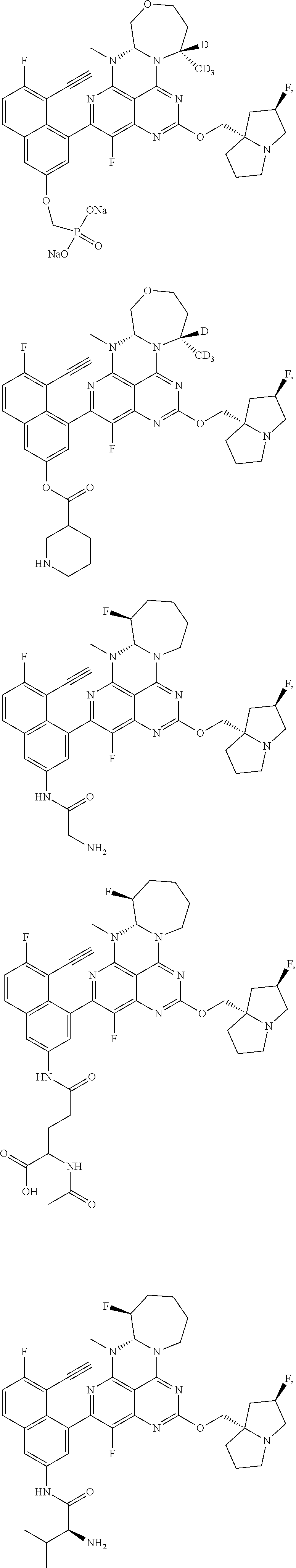

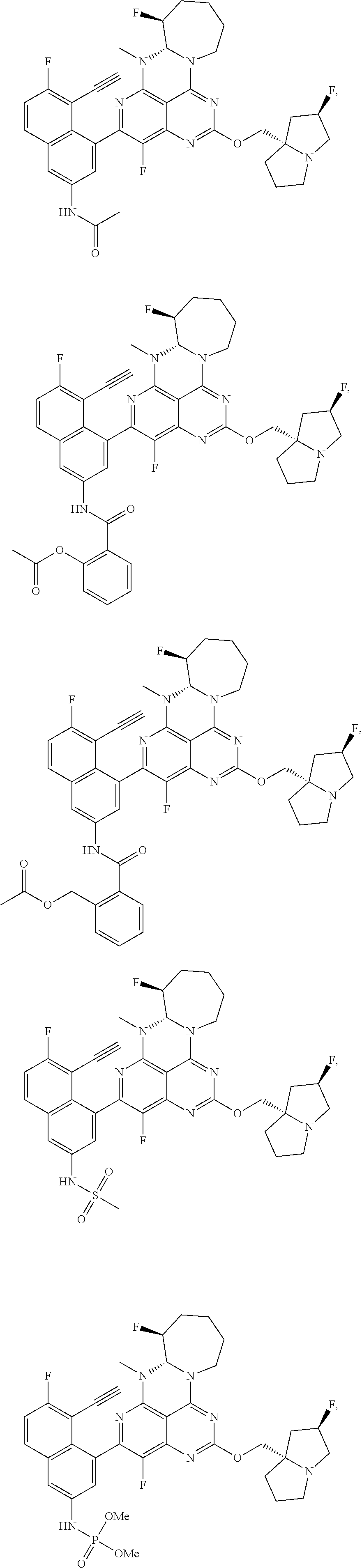

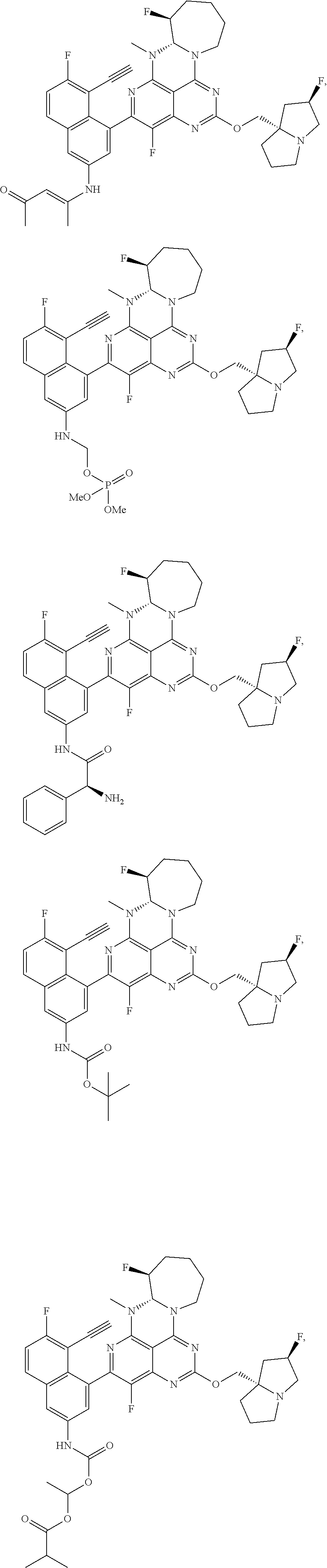

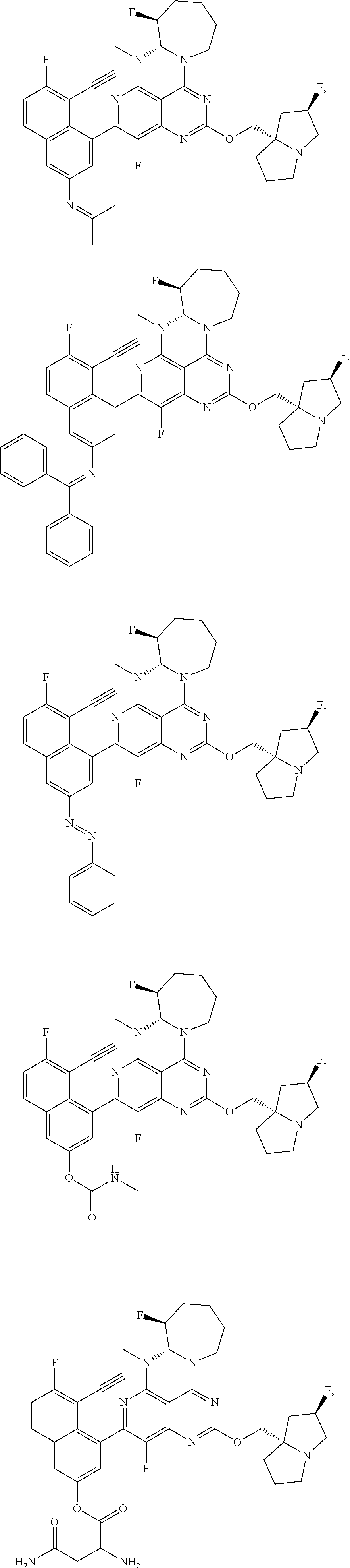

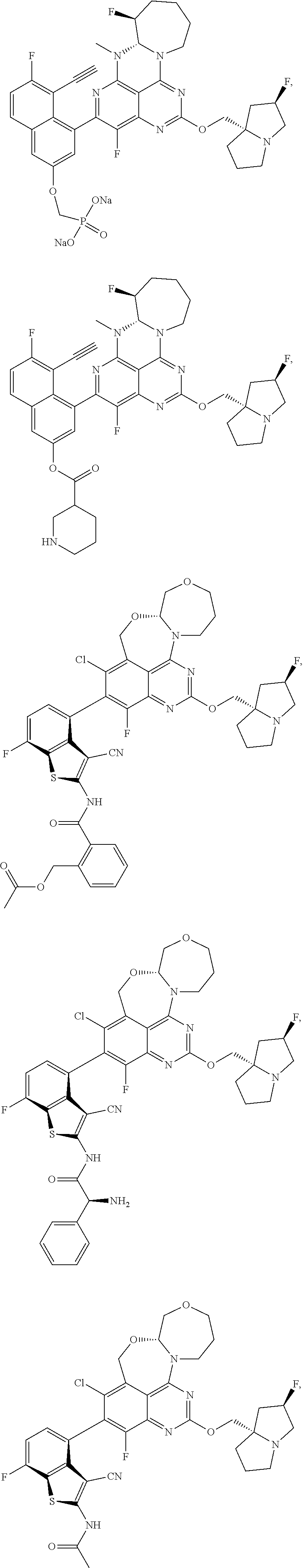

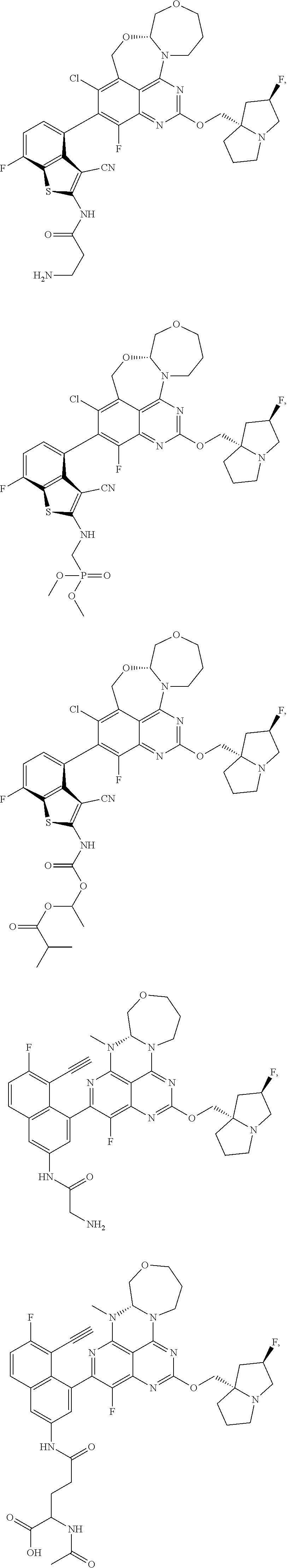

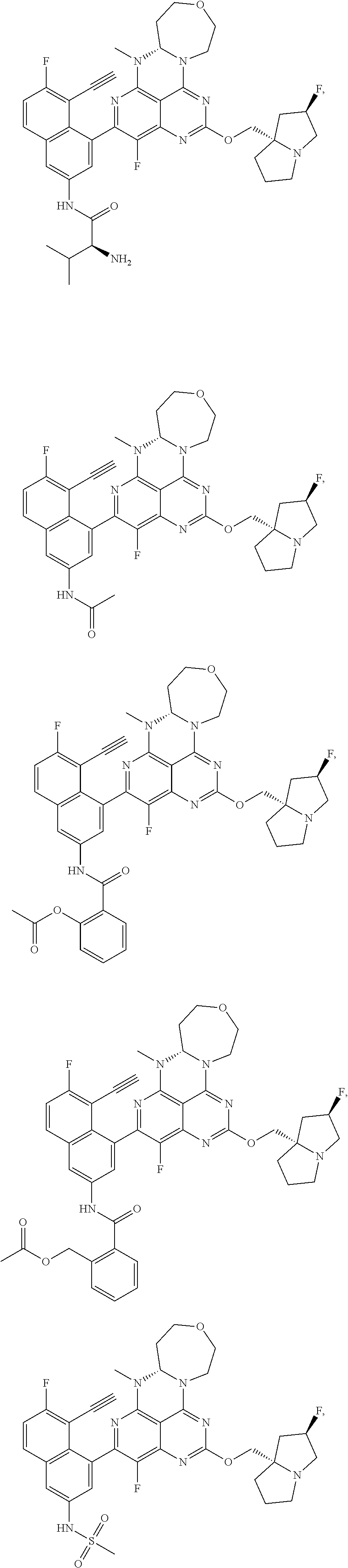

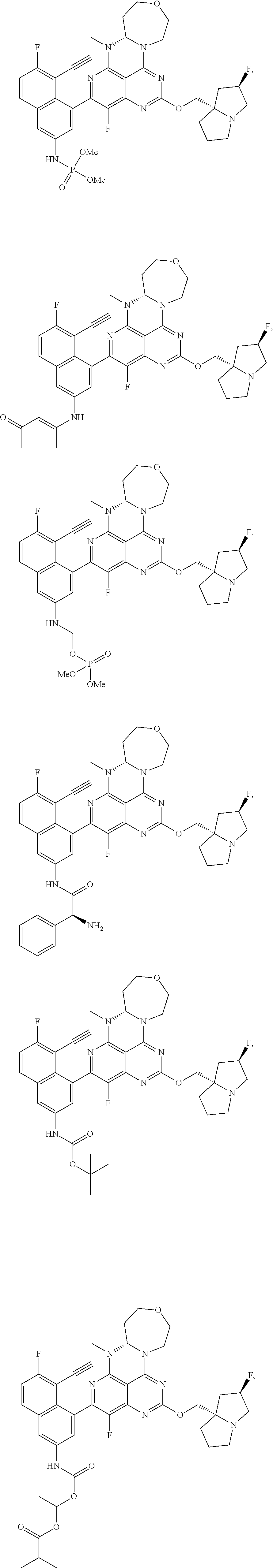

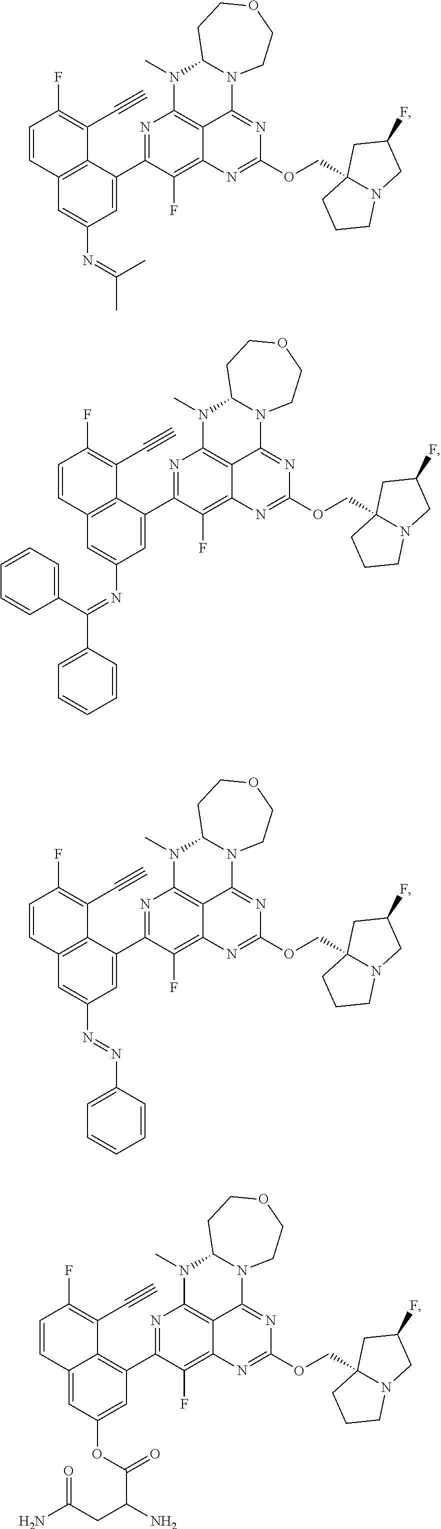 [SMILES: C#Cc1c(F)ccc2cc(N=C(C)C)cc(-c3nc4c5c(nc(OC[C@@]67CCCN6C[C@H](F)C7)nc5c3F)N3CCOCC[C@H]3N4C)c12 ; C#Cc1c(F)ccc2cc(N=C(c3ccccc3)c3ccccc3)cc(-c3nc4c5c(nc(OC[C@@]67CCCN6C[C@H](F)C7)nc5c3F)N3CCOCC[C@H]3N4C)c12 ; C#Cc1c(F)ccc2cc(/N=N/c3ccccc3)cc(-c3nc4c5c(nc(OC[C@@]67CCCN6C[C@H](F)C7)nc5c3F)N3CCOCC[C@H]3N4C)c12 ; C#Cc1c(F)ccc2cc(OC(=O)C(N)CC(N)=O)cc(-c3nc4c5c(nc(OC[C@@]67CCCN6C[C@H](F)C7)nc5c3F)N3CCOCC[C@H]3N4C)c12]

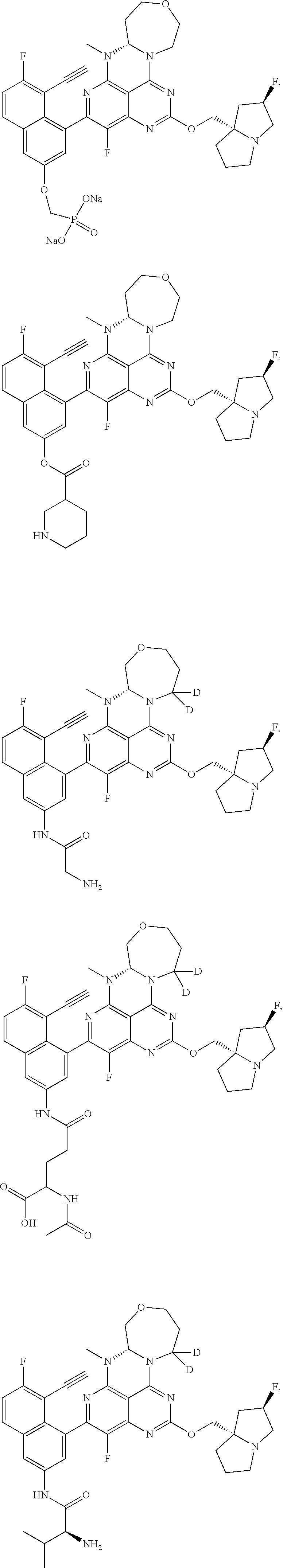

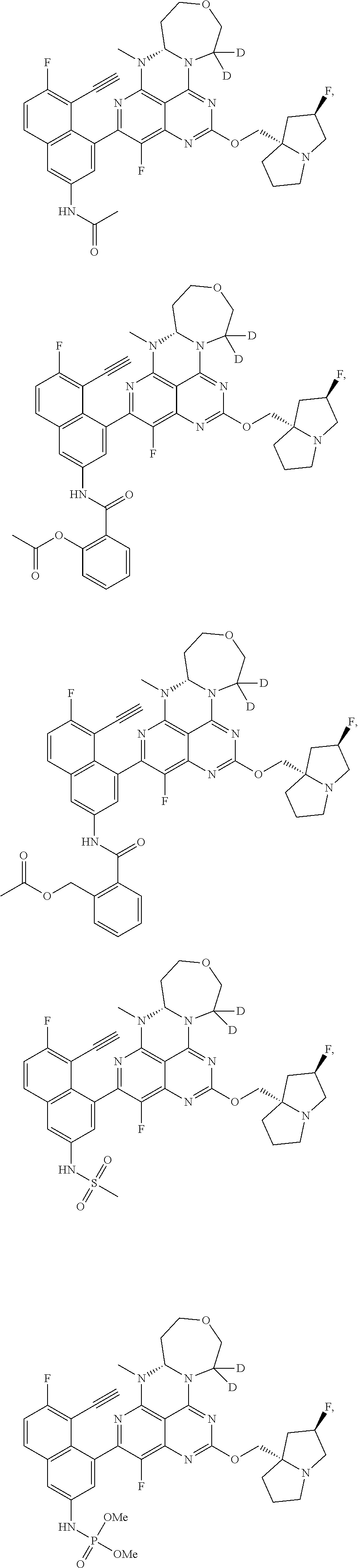

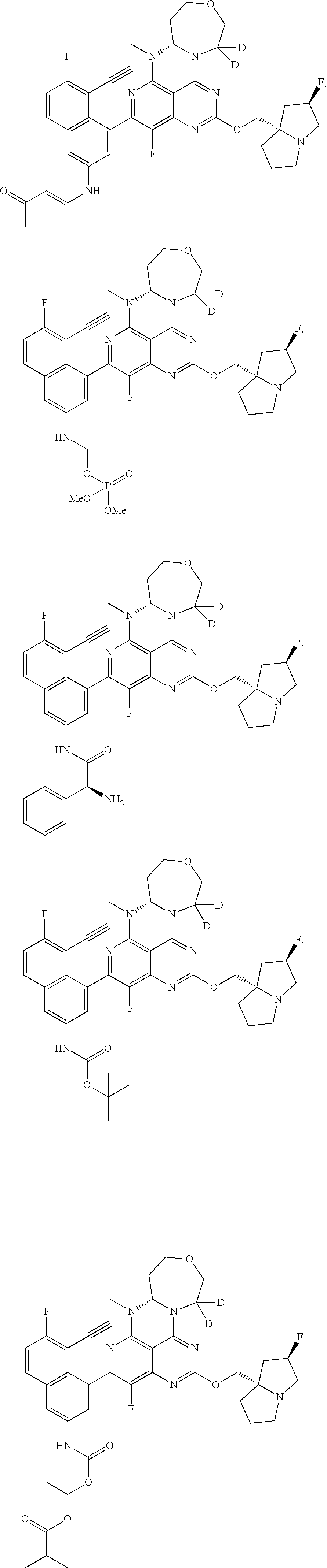

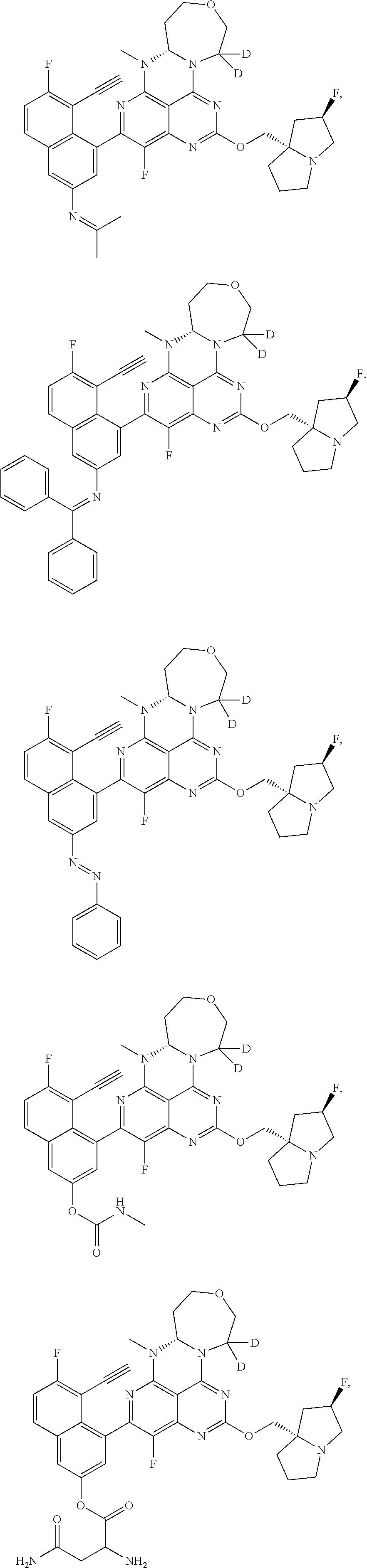

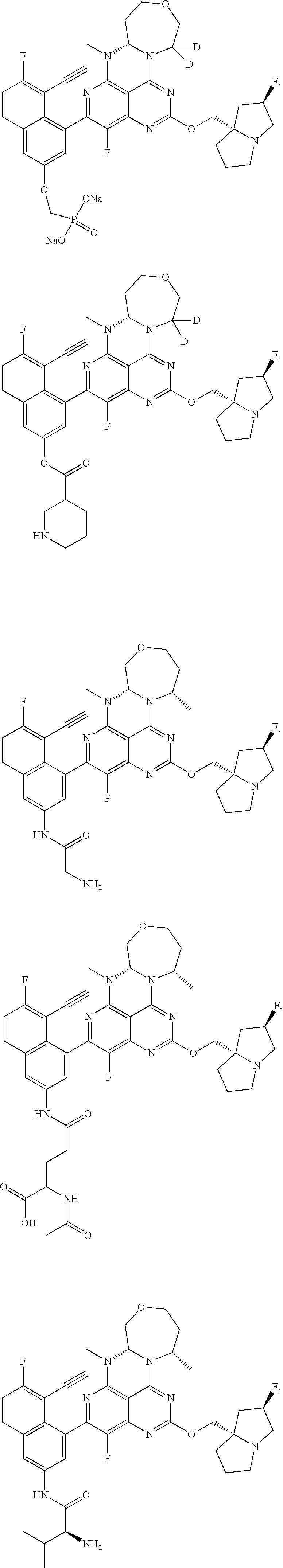

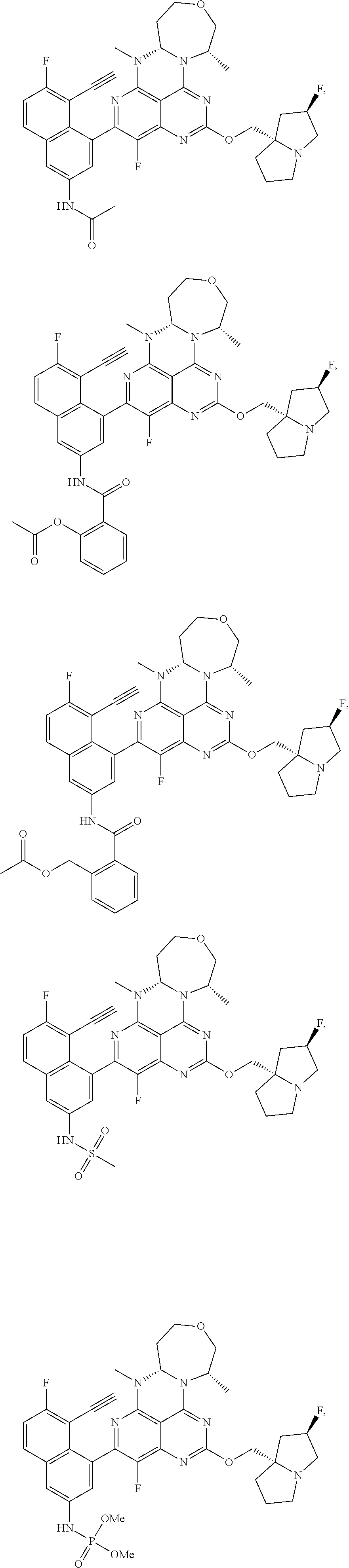

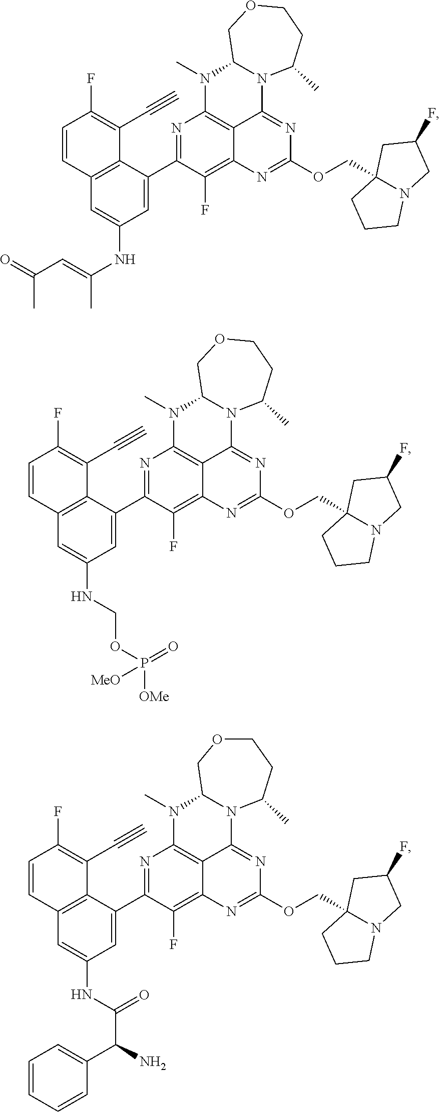 [SMILES: C#Cc1c(F)ccc2cc(N/C(C)=C/C(C)=O)cc(-c3nc4c5c(nc(OC[C@@]67CCCN6C[C@H](F)C7)nc5c3F)N3[C@@H](C)CCOC[C@H]3N4C)c12 ; C#Cc1c(F)ccc2cc(NCOP(=O)(OC)OC)cc(-c3nc4c5c(nc(OC[C@@]67CCCN6C[C@H](F)C7)nc5c3F)N3[C@@H](C)CCOC[C@H]3N4C)c12 ; C#Cc1c(F)ccc2cc(NC(=O)[C@@H](N)c3ccccc3)cc(-c3nc4c5c(nc(OC[C@@]67CCCN6C[C@H](F)C7)nc5c3F)N3[C@@H](C)CCOC[C@H]3N4C)c12]

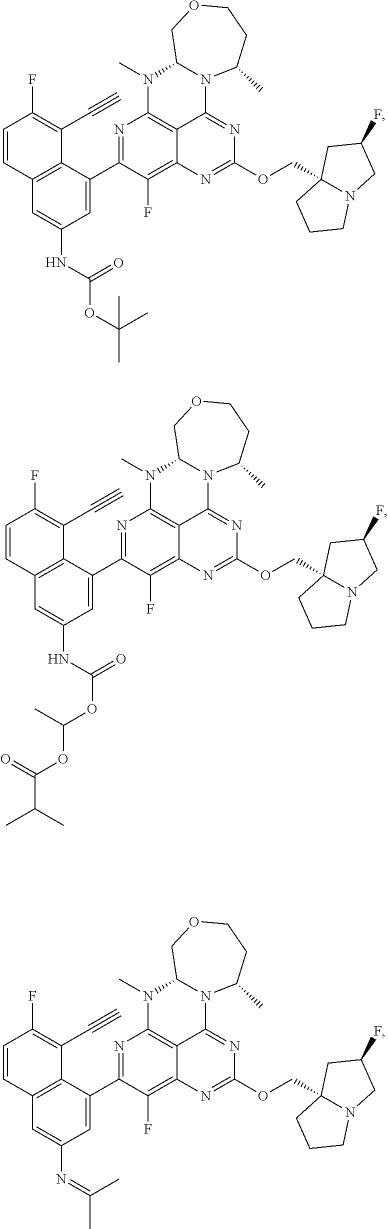 [SMILES: C#Cc1c(F)ccc2cc(NC(=O)OC(C)(C)C)cc(-c3nc4c5c(nc(OC[C@@]67CCCN6C[C@H](F)C7)nc5c3F)N3[C@@H](C)CCOC[C@H]3N4C)c12 ; C#Cc1c(F)ccc2cc(NC(=O)OC(C)OC(=O)C(C)C)cc(-c3nc4c5c(nc(OC[C@@]67CCCN6C[C@H](F)C7)nc5c3F)N3[C@@H](C)CCOC[C@H]3N4C)c12 ; C#Cc1c(F)ccc2cc(N=C(C)C)cc(-c3nc4c5c(nc(OC[C@@]67CCCN6C[C@H](F)C7)nc5c3F)N3[C@@H](C)CCOC[C@H]3N4C)c12]

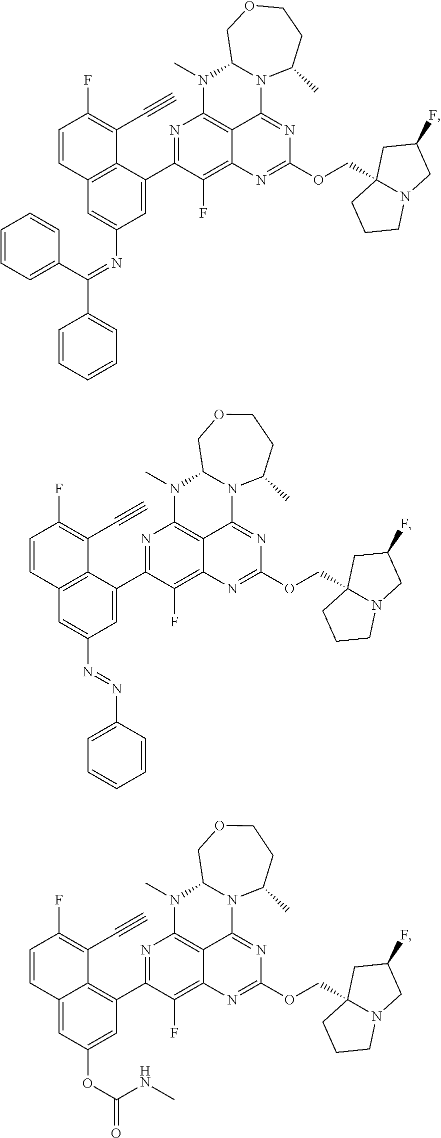 [SMILES: C#Cc1c(F)ccc2cc(N=C(c3ccccc3)c3ccccc3)cc(-c3nc4c5c(nc(OC[C@@]67CCCN6C[C@H](F)C7)nc5c3F)N3[C@@H](C)CCOC[C@H]3N4C)c12 ; C#Cc1c(F)ccc2cc(/N=N/c3ccccc3)cc(-c3nc4c5c(nc(OC[C@@]67CCCN6C[C@H](F)C7)nc5c3F)N3[C@@H](C)CCOC[C@H]3N4C)c12 ; C#Cc1c(F)ccc2cc(OC(=O)NC)cc(-c3nc4c5c(nc(OC[C@@]67CCCN6C[C@H](F)C7)nc5c3F)N3[C@@H](C)CCOC[C@H]3N4C)c12]

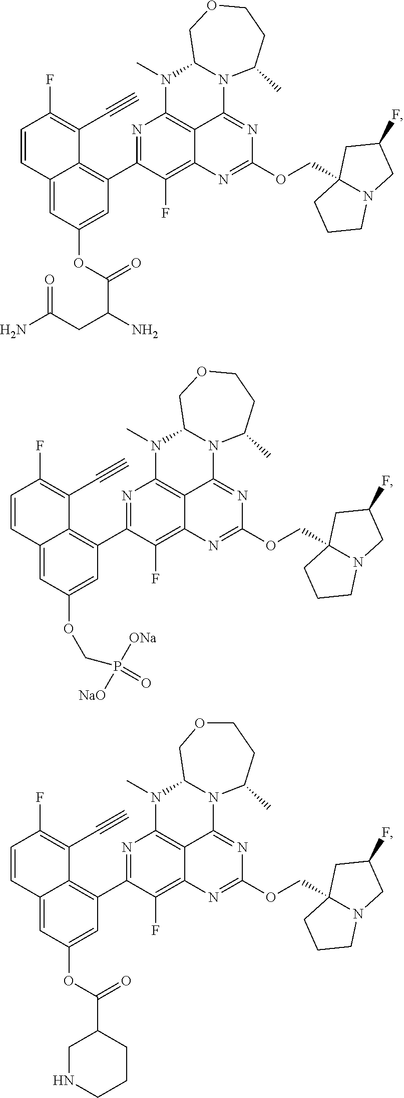

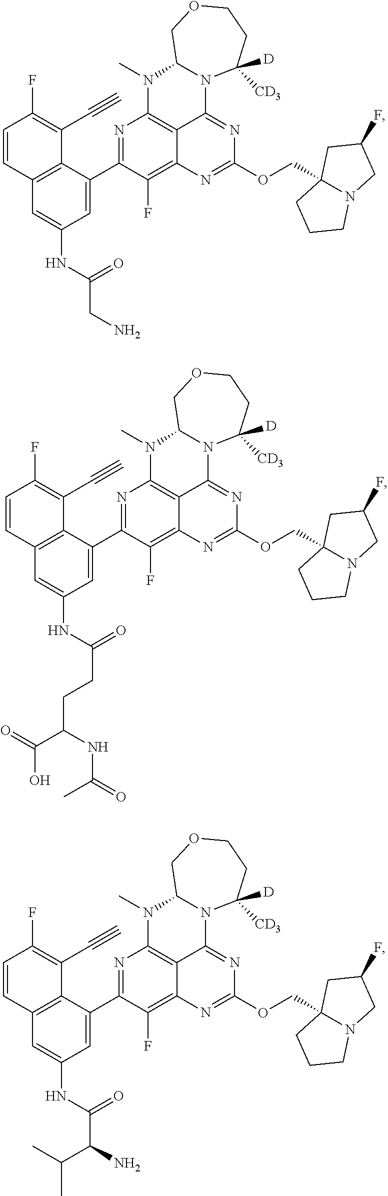 [SMILES: [2H]C([2H])([2H])[C@@]1([2H])CCOC[C@H]2N(C)c3nc(-c4cc(NC(=O)CN)cc5ccc(F)c(C#C)c45)c(F)c4nc(OC[C@@]56CCCN5C[C@H](F)C6)nc(c34)N21 ; [2H]C([2H])([2H])[C@@]1([2H])CCOC[C@H]2N(C)c3nc(-c4cc(NC(=O)CCC(NC(C)=O)C(=O)O)cc5ccc(F)c(C#C)c45)c(F)c4nc(OC[C@@]56CCCN5C[C@H](F)C6)nc(c34)N21 ; [2H]C([2H])([2H])[C@@]1([2H])CCOC[C@H]2N(C)c3nc(-c4cc(NC(=O)[C@@H](N)C(C)C)cc5ccc(F)c(C#C)c45)c(F)c4nc(OC[C@@]56CCCN5C[C@H](F)C6)nc(c34)N21]

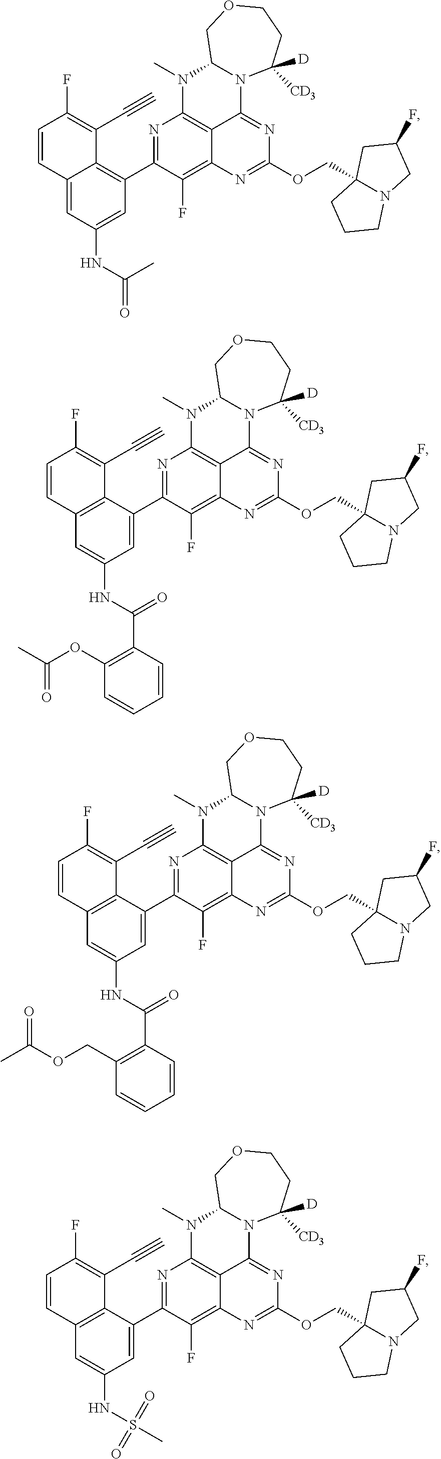

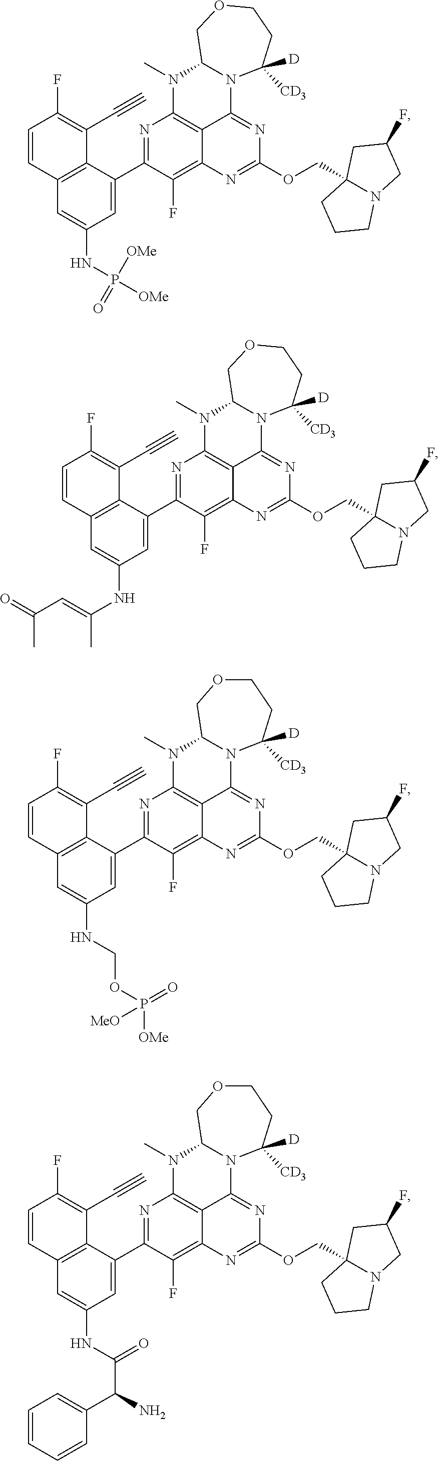

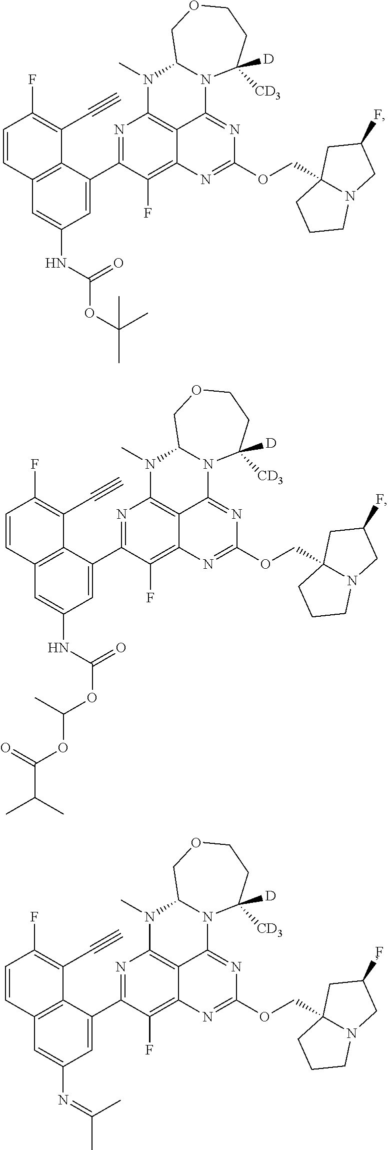 [SMILES: [2H]C([2H])([2H])[C@@]1([2H])CCOC[C@H]2N(C)c3nc(-c4cc(NC(=O)OC(C)(C)C)cc5ccc(F)c(C#C)c45)c(F)c4nc(OC[C@@]56CCCN5C[C@H](F)C6)nc(c34)N21 ; [2H]C([2H])([2H])[C@@]1([2H])CCOC[C@H]2N(C)c3nc(-c4cc(NC(=O)OC(C)OC(=O)C(C)C)cc5ccc(F)c(C#C)c45)c(F)c4nc(OC[C@@]56CCCN5C[C@H](F)C6)nc(c34)N21 ; [2H]C([2H])([2H])[C@@]1([2H])CCOC[C@H]2N(C)c3nc(-c4cc(N=C(C)C)cc5ccc(F)c(C#C)c45)c(F)c4nc(OC[C@@]56CCCN5C[C@H](F)C6)nc(c34)N21]

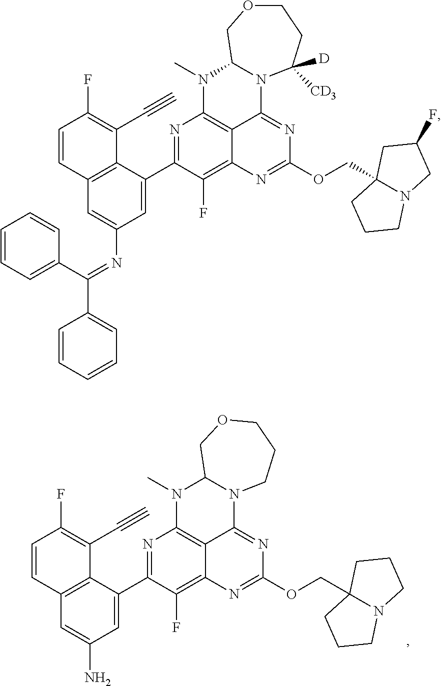 [SMILES: [2H]C([2H])([2H])[C@@]1([2H])CCOC[C@H]2N(C)c3nc(-c4cc(N=C(c5ccccc5)c5ccccc5)cc5ccc(F)c(C#C)c45)c(F)c4nc(OC[C@@]56CCCN5C[C@H](F)C6)nc(c34)N21 ; C#Cc1c(F)ccc2cc(N)cc(-c3nc4c5c(nc(OCC67CCCN6CCC7)nc5c3F)N3CCCOCC3N4C)c12]

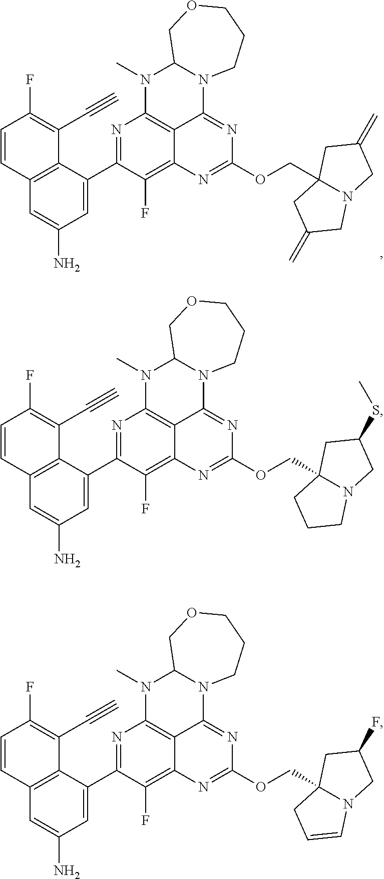 [SMILES: C#Cc1c(F)ccc2cc(N)cc(-c3nc4c5c(nc(OCC67CC(=C)CN6CC(=C)C7)nc5c3F)N3CCCOCC3N4C)c12 ; C#Cc1c(F)ccc2cc(N)cc(-c3nc4c5c(nc(OC[C@@]67CCCN6C[C@H](SC)C7)nc5c3F)N3CCCOCC3N4C)c12 ; C#Cc1c(F)ccc2cc(N)cc(-c3nc4c5c(nc(OC[C@@]67CC=CN6C[C@H](F)C7)nc5c3F)N3CCCOCC3N4C)c12]

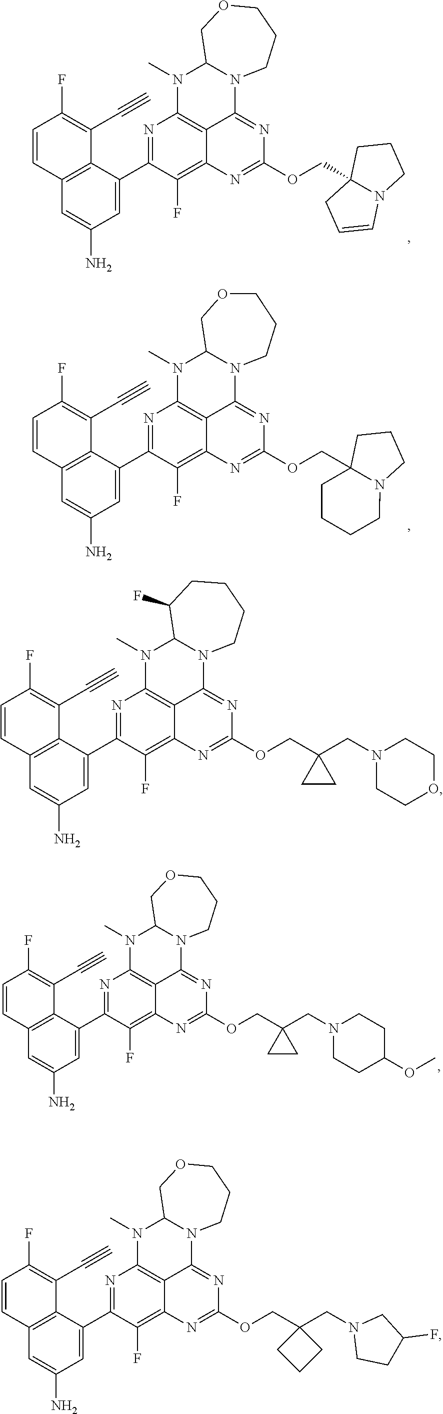 [SMILES: C#Cc1c(F)ccc2cc(N)cc(-c3nc4c5c(nc(OC[C@@]67CC=CN6CCC7)nc5c3F)N3CCCOCC3N4C)c12 ; C#Cc1c(F)ccc2cc(N)cc(-c3nc4c5c(nc(OCC67CCCCN6CCC7)nc5c3F)N3CCCOCC3N4C)c12 ; C#Cc1c(F)ccc2cc(N)cc(-c3nc4c5c(nc(OCC6(CN7CCOCC7)CC6)nc5c3F)N3CCCC[C@H](F)C3N4C)c12 ; C#Cc1c(F)ccc2cc(N)cc(-c3nc4c5c(nc(OCC6(CN7CCC(OC)CC7)CC6)nc5c3F)N3CCCOCC3N4C)c12 ; C#Cc1c(F)ccc2cc(N)cc(-c3nc4c5c(nc(OCC6(CN7CCC(F)C7)CCC6)nc5c3F)N3CCCOCC3N4C)c12]

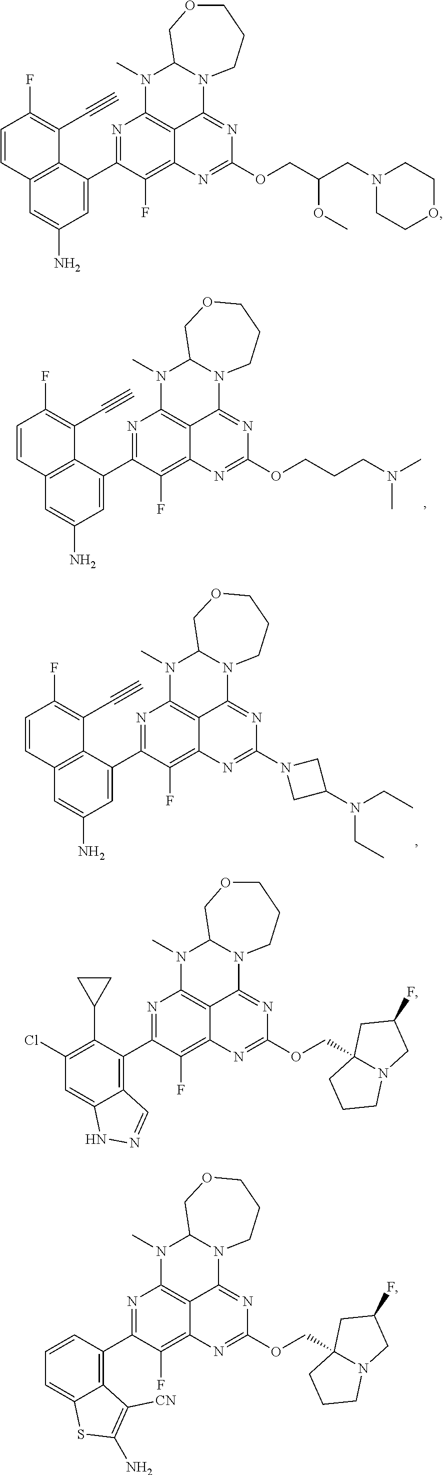 [SMILES: C#Cc1c(F)ccc2cc(N)cc(-c3nc4c5c(nc(OCC(CN6CCOCC6)OC)nc5c3F)N3CCCOCC3N4C)c12 ; C#Cc1c(F)ccc2cc(N)cc(-c3nc4c5c(nc(OCCCN(C)C)nc5c3F)N3CCCOCC3N4C)c12 ; C#Cc1c(F)ccc2cc(N)cc(-c3nc4c5c(nc(N6CC(N(CC)CC)C6)nc5c3F)N3CCCOCC3N4C)c12 ; CN1c2nc(-c3c(C4CC4)c(Cl)cc4[nH]ncc34)c(F)c3nc(OC[C@@]45CCCN4C[C@H](F)C5)nc(c23)N2CCCOCC12 ; CN1c2nc(-c3cccc4sc(N)c(C#N)c34)c(F)c3nc(OC[C@@]45CCCN4C[C@H](F)C5)nc(c23)N2CCCOCC12]

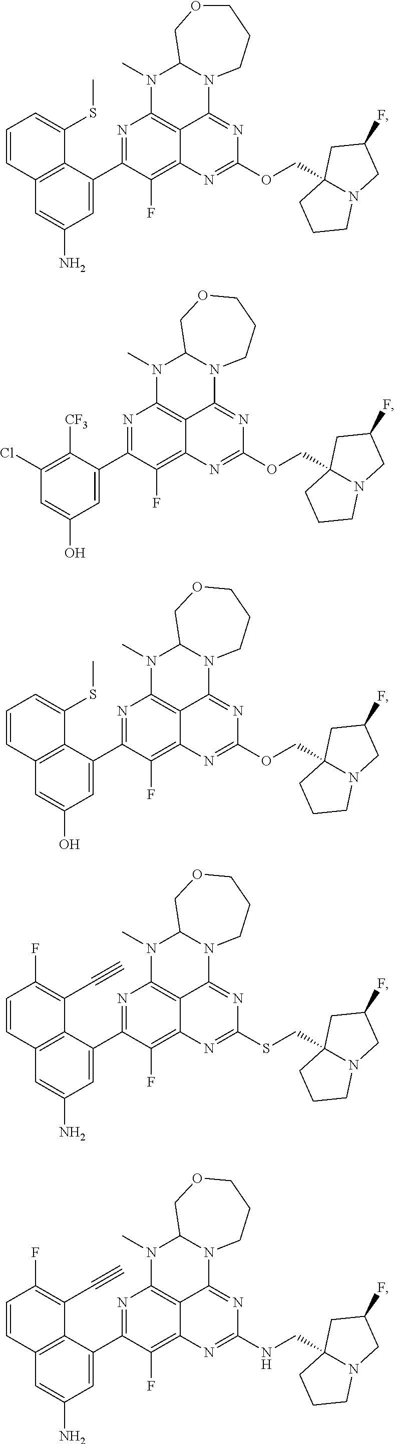 [SMILES: CSc1cccc2cc(N)cc(-c3nc4c5c(nc(OC[C@@]67CCCN6C[C@H](F)C7)nc5c3F)N3CCCOCC3N4C)c12 ; CN1c2nc(-c3cc(O)cc(Cl)c3C(F)(F)F)c(F)c3nc(OC[C@@]45CCCN4C[C@H](F)C5)nc(c23)N2CCCOCC12 ; CSc1cccc2cc(O)cc(-c3nc4c5c(nc(OC[C@@]67CCCN6C[C@H](F)C7)nc5c3F)N3CCCOCC3N4C)c12 ; C#Cc1c(F)ccc2cc(N)cc(-c3nc4c5c(nc(SC[C@@]67CCCN6C[C@H](F)C7)nc5c3F)N3CCCOCC3N4C)c12 ; C#Cc1c(F)ccc2cc(N)cc(-c3nc4c5c(nc(NC[C@@]67CCCN6C[C@H](F)C7)nc5c3F)N3CCCOCC3N4C)c12]

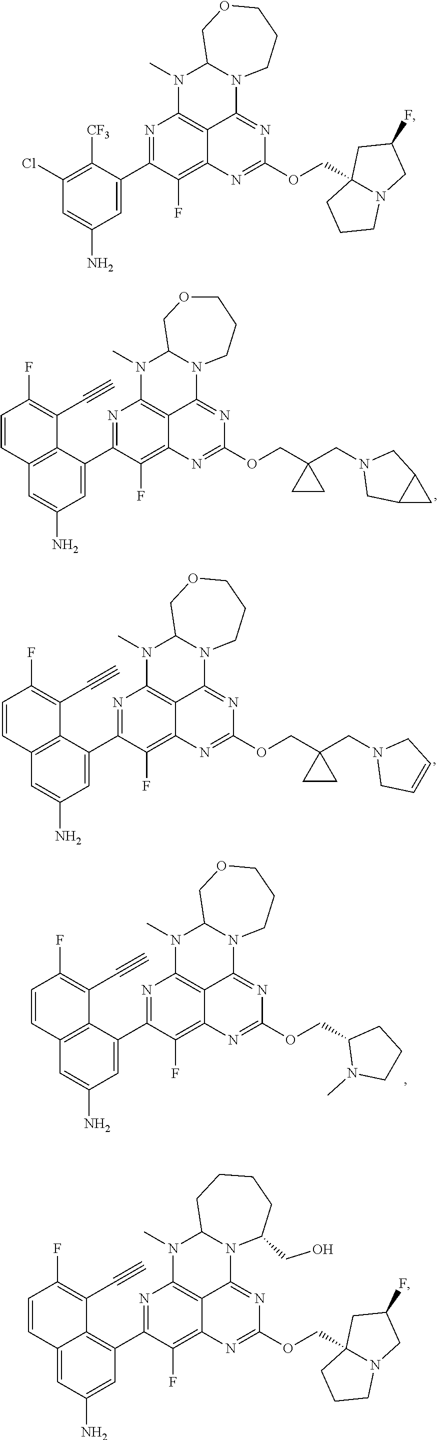 [SMILES: CN1c2nc(-c3cc(N)cc(Cl)c3C(F)(F)F)c(F)c3nc(OC[C@@]45CCCN4C[C@H](F)C5)nc(c23)N2CCCOCC12 ; C#Cc1c(F)ccc2cc(N)cc(-c3nc4c5c(nc(OCC6(CN7CC8CC8C7)CC6)nc5c3F)N3CCCOCC3N4C)c12 ; C#Cc1c(F)ccc2cc(N)cc(-c3nc4c5c(nc(OCC6(CN7CC=CC7)CC6)nc5c3F)N3CCCOCC3N4C)c12 ; C#Cc1c(F)ccc2cc(N)cc(-c3nc4c5c(nc(OC[C@@H]6CCCN6C)nc5c3F)N3CCCOCC3N4C)c12 ; C#Cc1c(F)ccc2cc(N)cc(-c3nc4c5c(nc(OC[C@@]67CCCN6C[C@H](F)C7)nc5c3F)N3C(CCCC[C@@H]3CO)N4C)c12]

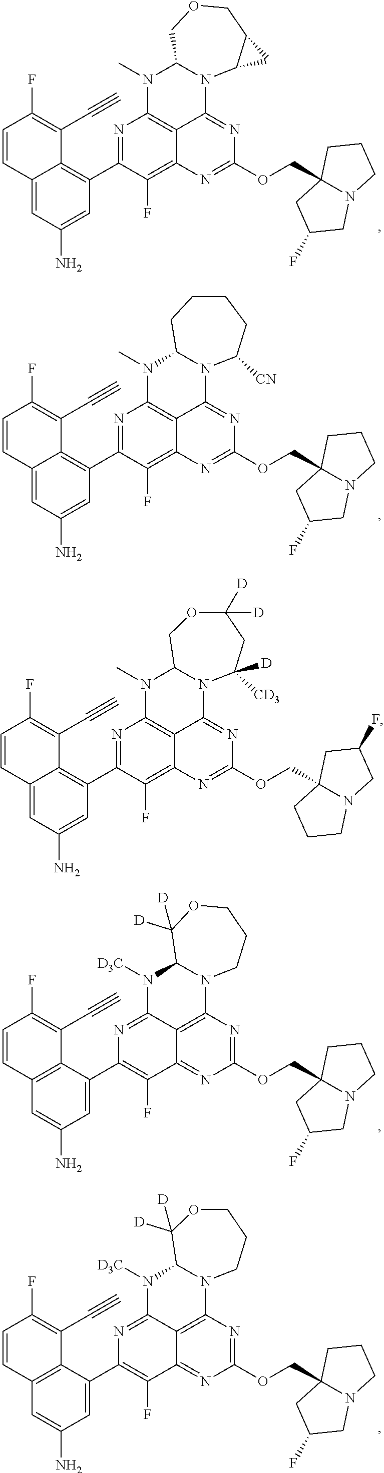

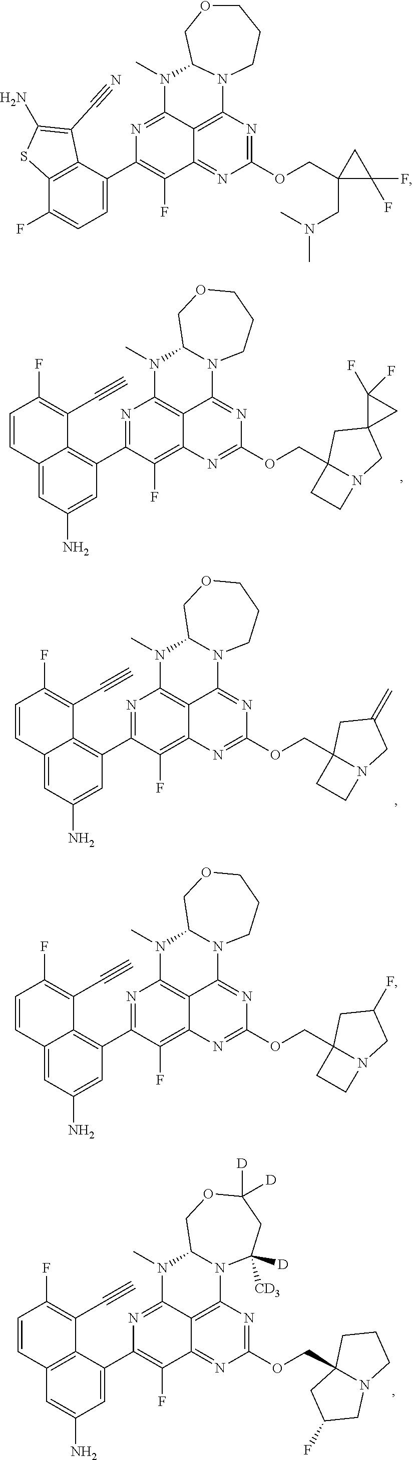 [SMILES: CN(C)CC1(COc2nc3c4c(nc(-c5ccc(F)c6sc(N)c(C#N)c56)c(F)c4n2)N(C)[C@@H]2COCCCN32)CC1(F)F ; C#Cc1c(F)ccc2cc(N)cc(-c3nc4c5c(nc(OCC67CCN6CC6(C7)CC6(F)F)nc5c3F)N3CCCOC[C@H]3N4C)c12 ; C#Cc1c(F)ccc2cc(N)cc(-c3nc4c5c(nc(OCC67CCN6CC(=C)C7)nc5c3F)N3CCCOC[C@H]3N4C)c12 ; C#Cc1c(F)ccc2cc(N)cc(-c3nc4c5c(nc(OCC67CCN6CC(F)C7)nc5c3F)N3CCCOC[C@H]3N4C)c12 ; [2H]C1([2H])C[C@]([2H])(C([2H])([2H])[2H])N2c3nc(OC[C@@]45CCCN4C[C@H](F)C5)nc4c(F)c(-c5cc(N)cc6ccc(F)c(C#C)c56)nc(c34)N(C)[C@@H]2CO1]

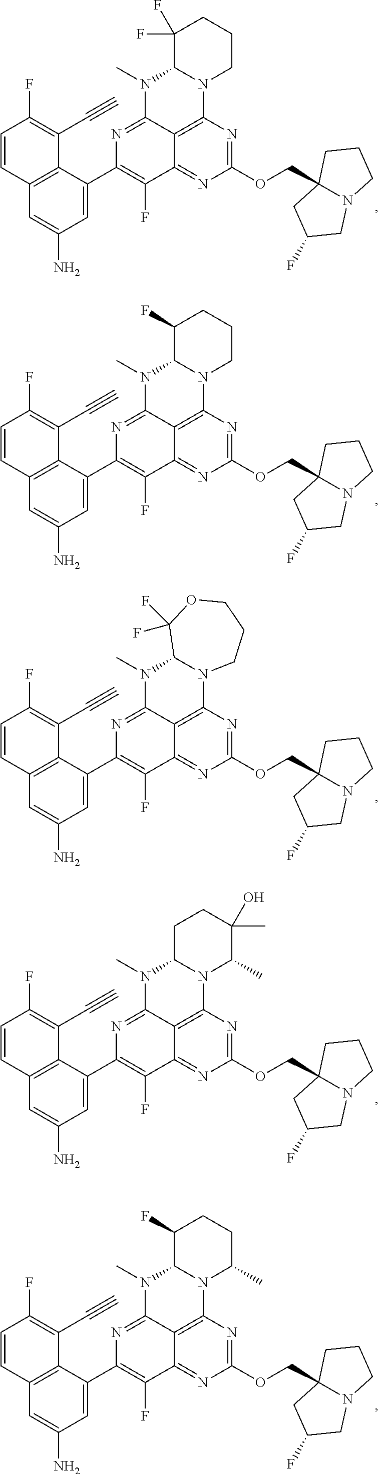 [SMILES: C#Cc1c(F)ccc2cc(N)cc(-c3nc4c5c(nc(OC[C@@]67CCCN6C[C@H](F)C7)nc5c3F)N3CCCC(F)(F)[C@H]3N4C)c12 ; C#Cc1c(F)ccc2cc(N)cc(-c3nc4c5c(nc(OC[C@@]67CCCN6C[C@H](F)C7)nc5c3F)N3CCC[C@H](F)[C@H]3N4C)c12 ; C#Cc1c(F)ccc2cc(N)cc(-c3nc4c5c(nc(OC[C@@]67CCCN6C[C@H](F)C7)nc5c3F)N3CCCOC(F)(F)[C@H]3N4C)c12 ; C#Cc1c(F)ccc2cc(N)cc(-c3nc4c5c(nc(OC[C@@]67CCCN6C[C@H](F)C7)nc5c3F)N3[C@@H](C)C(C)(O)CC[C@H]3N4C)c12 ; C#Cc1c(F)ccc2cc(N)cc(-c3nc4c5c(nc(OC[C@@]67CCCN6C[C@H](F)C7)nc5c3F)N3[C@@H](C)CC[C@H](F)[C@H]3N4C)c12]

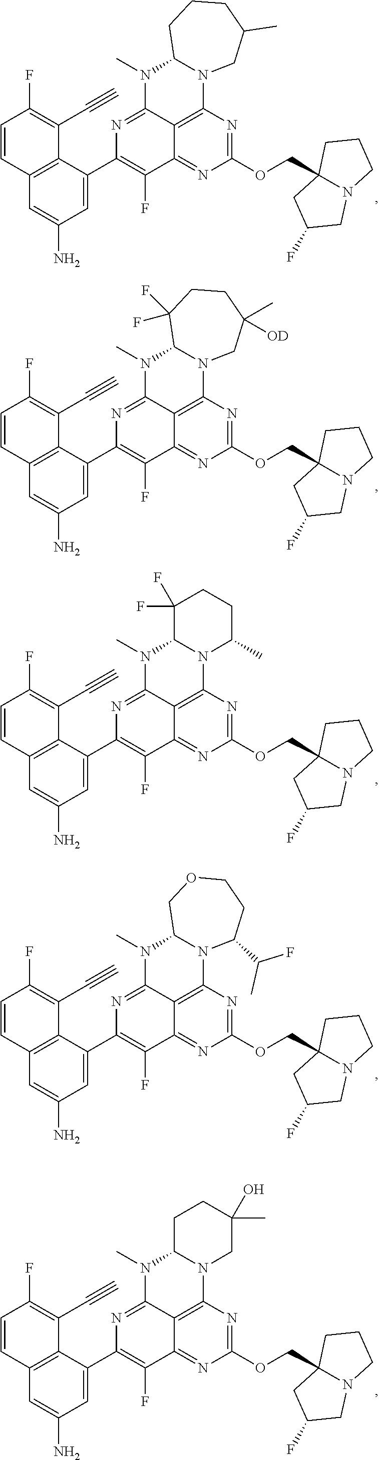

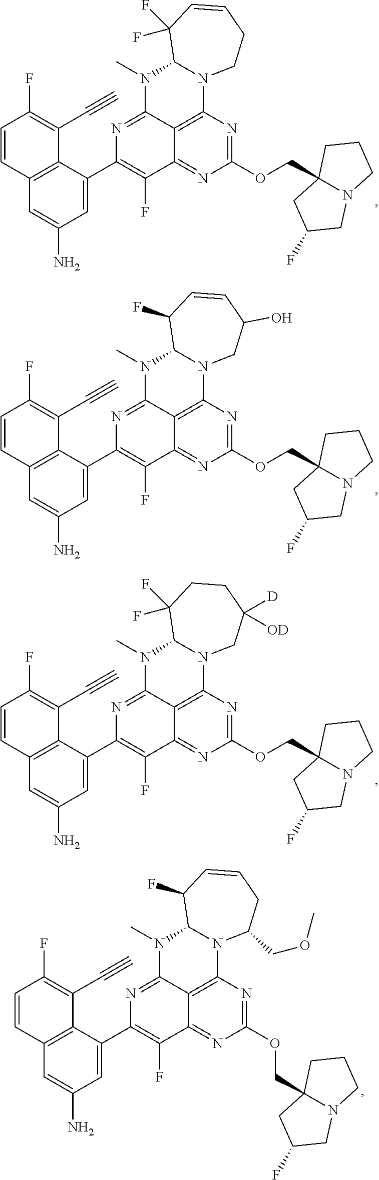

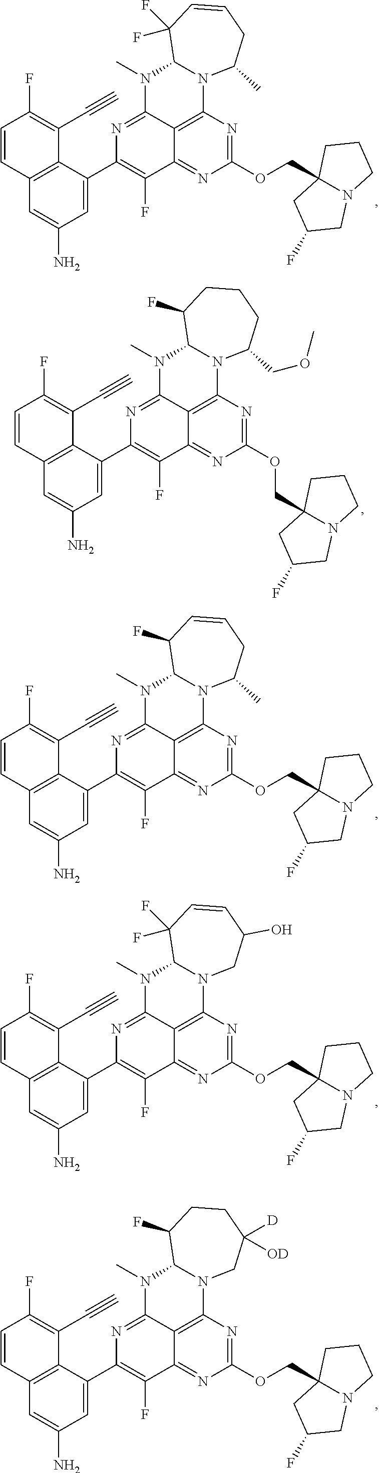

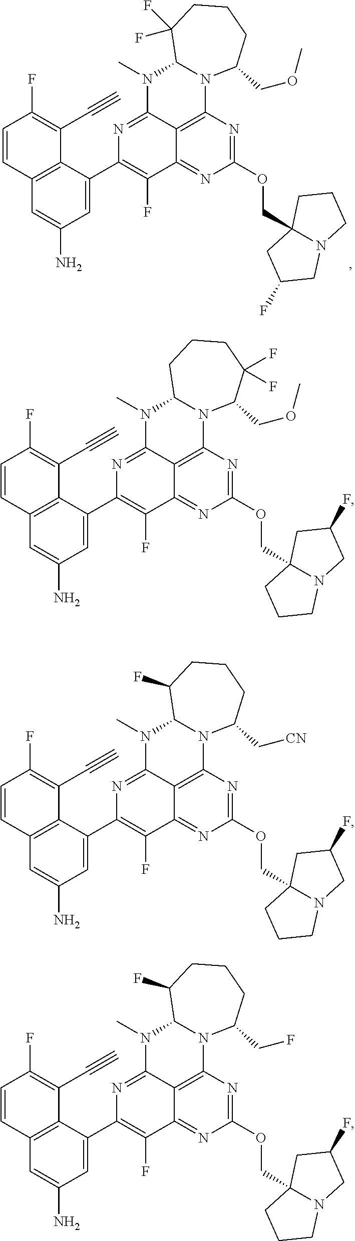

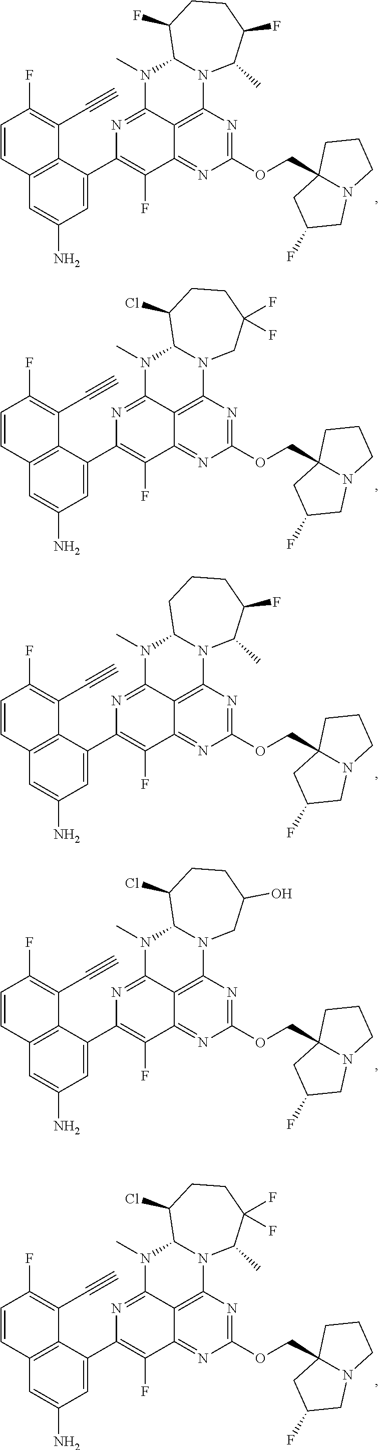

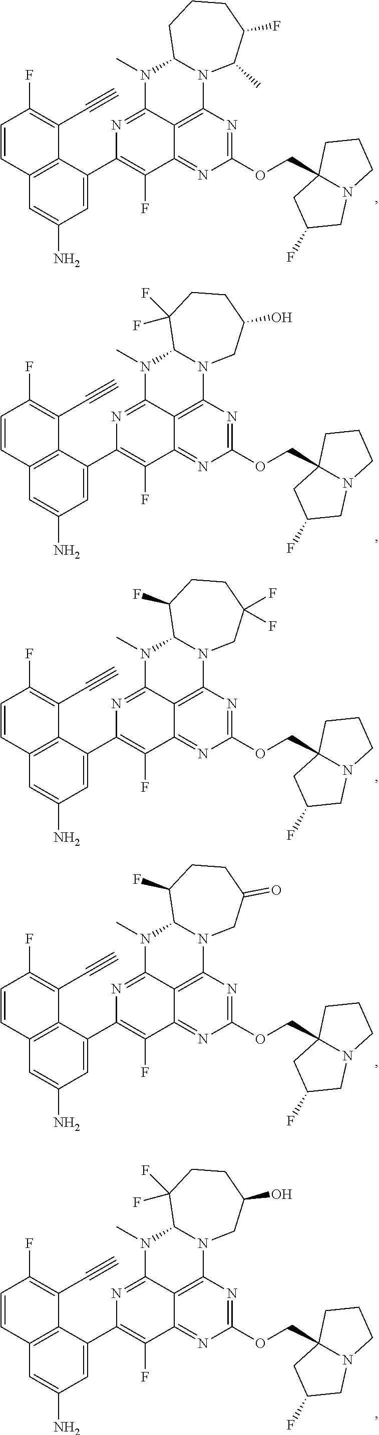

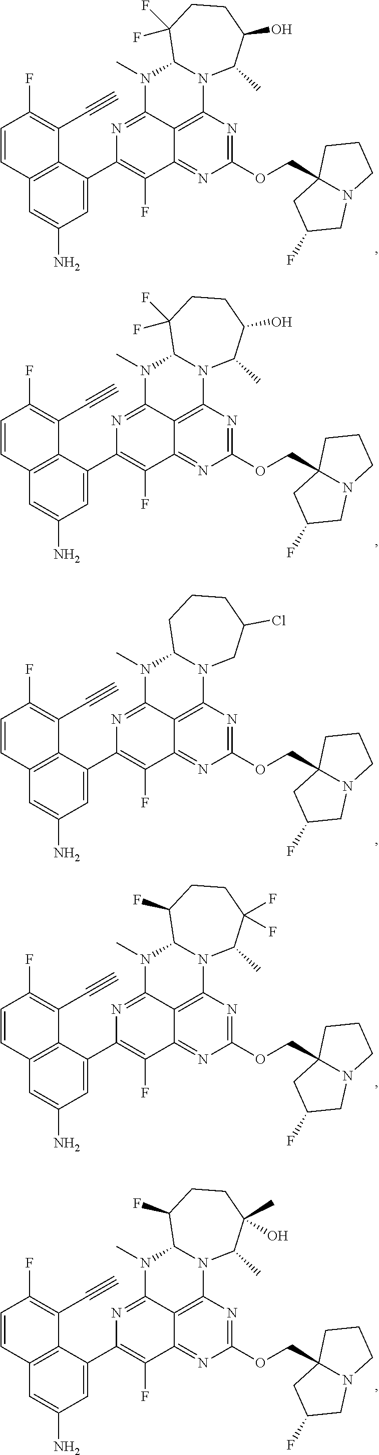

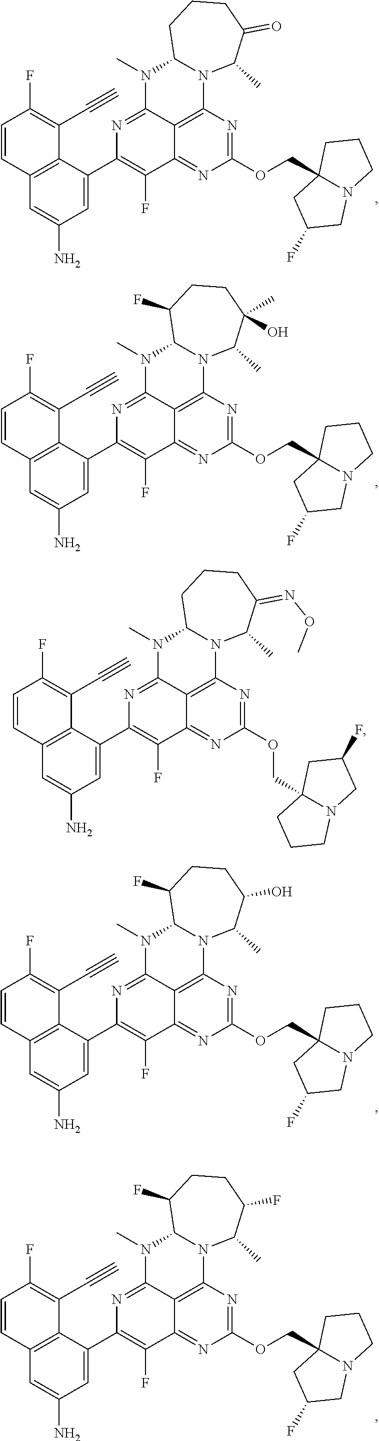

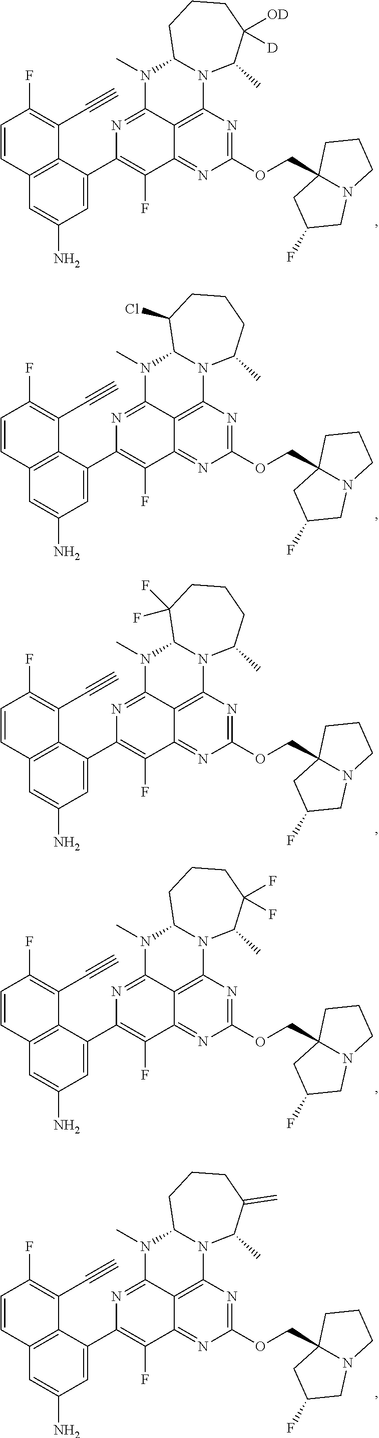

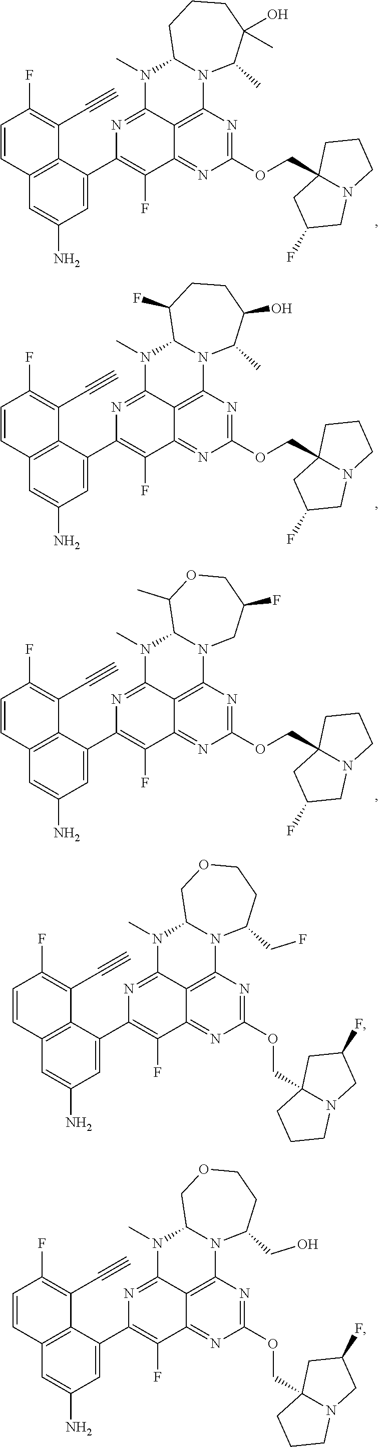

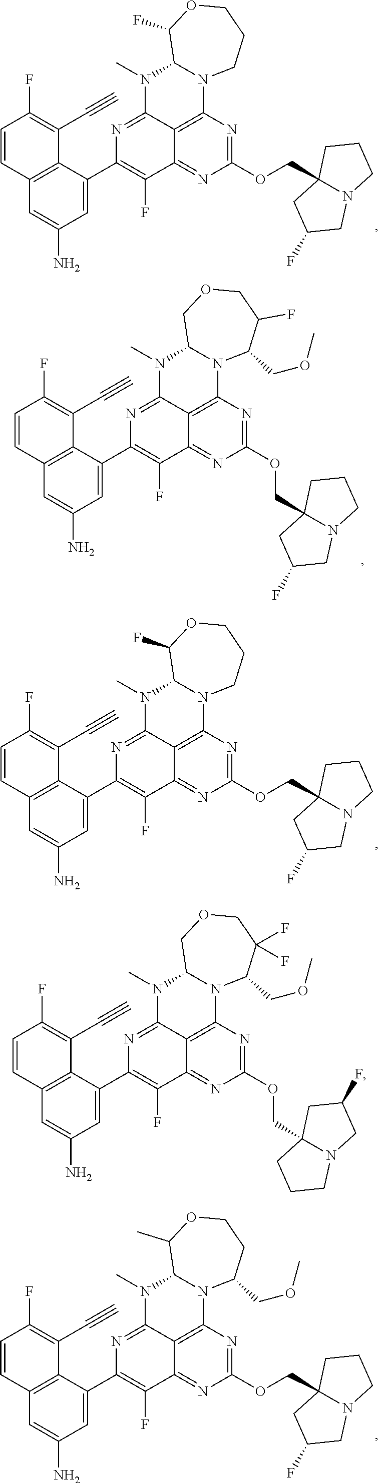

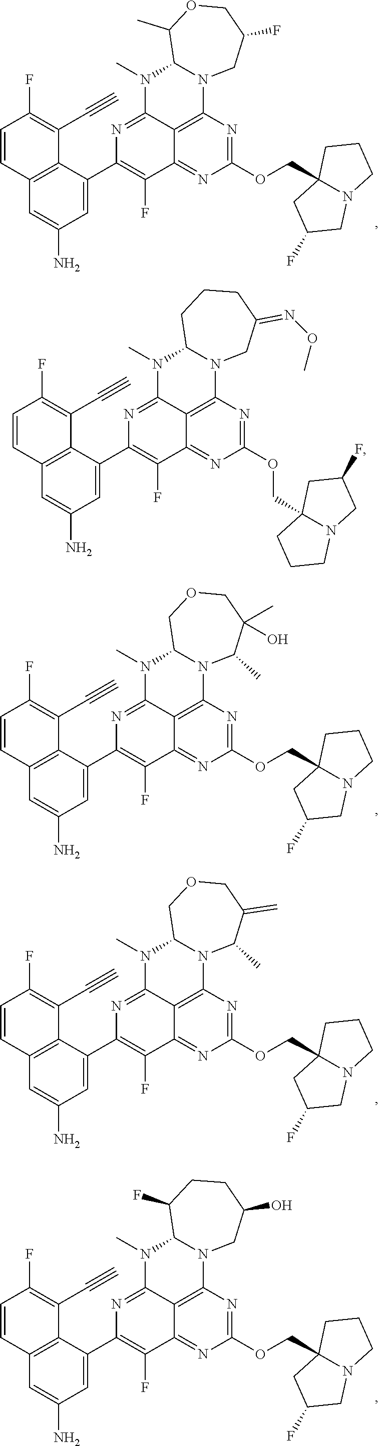

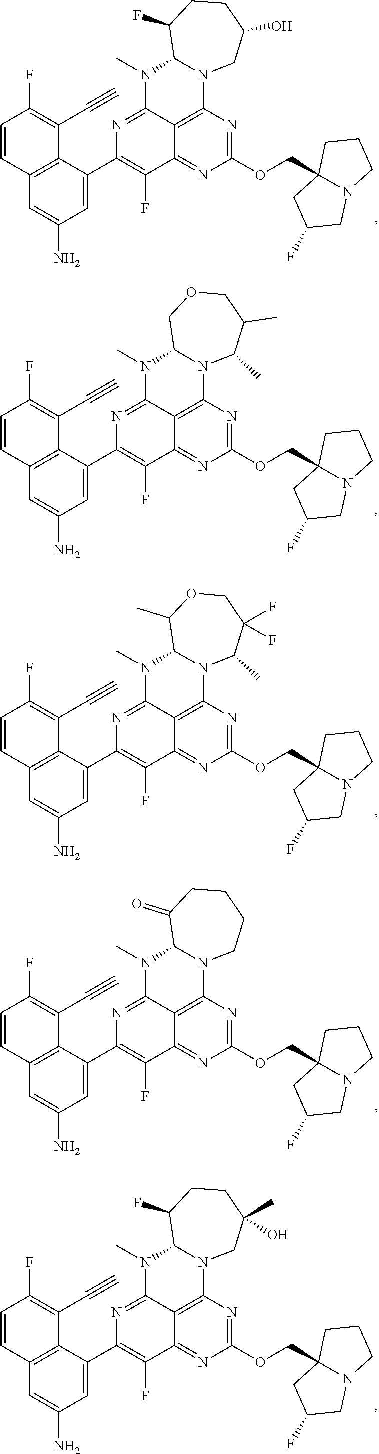

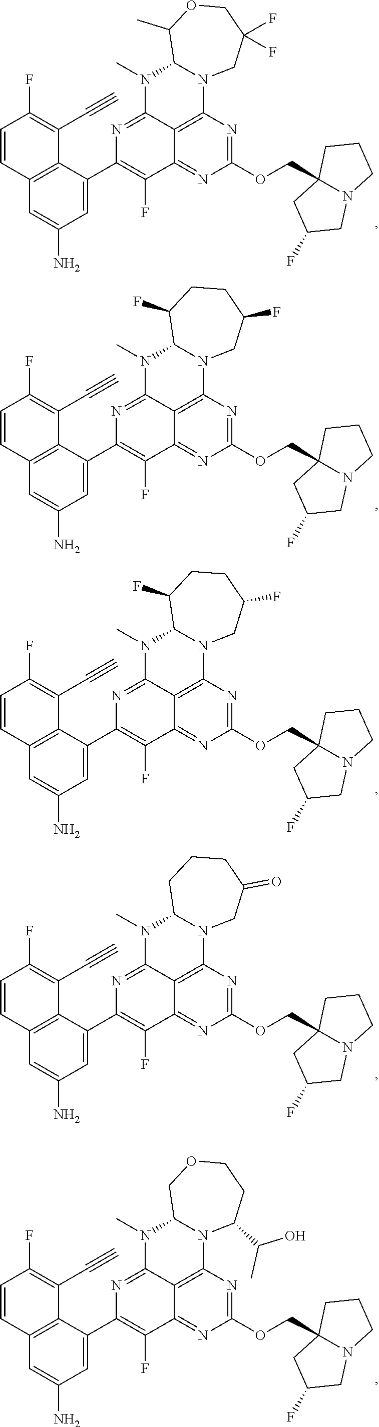

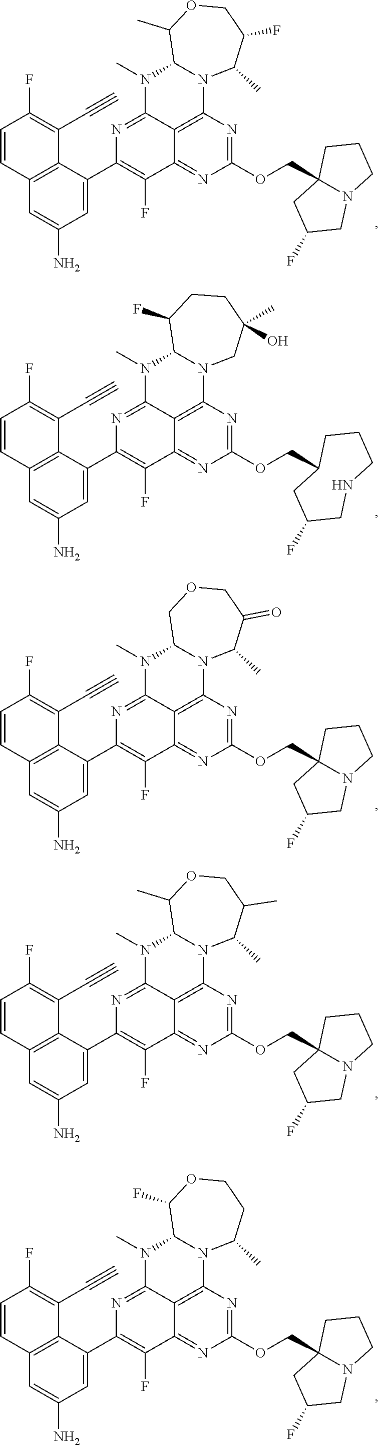

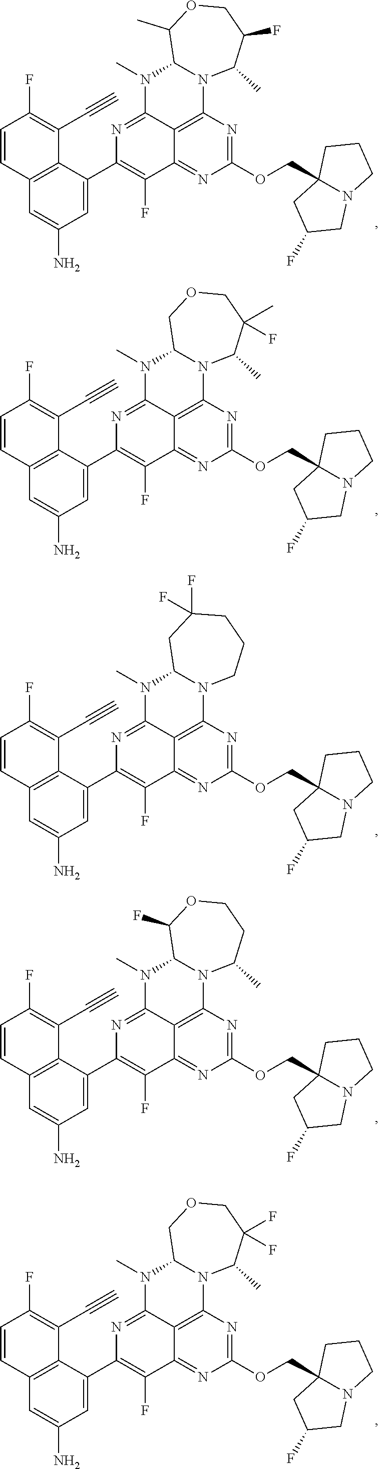

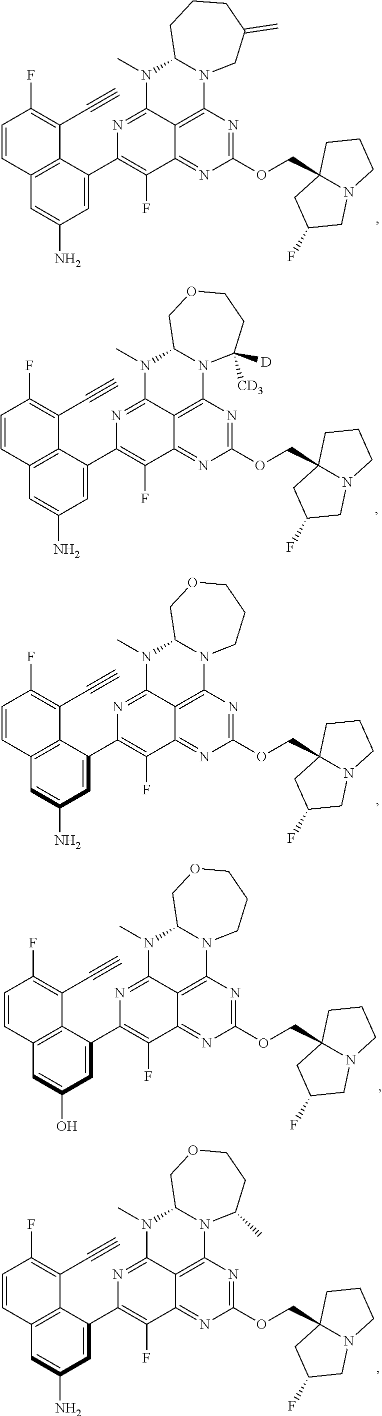

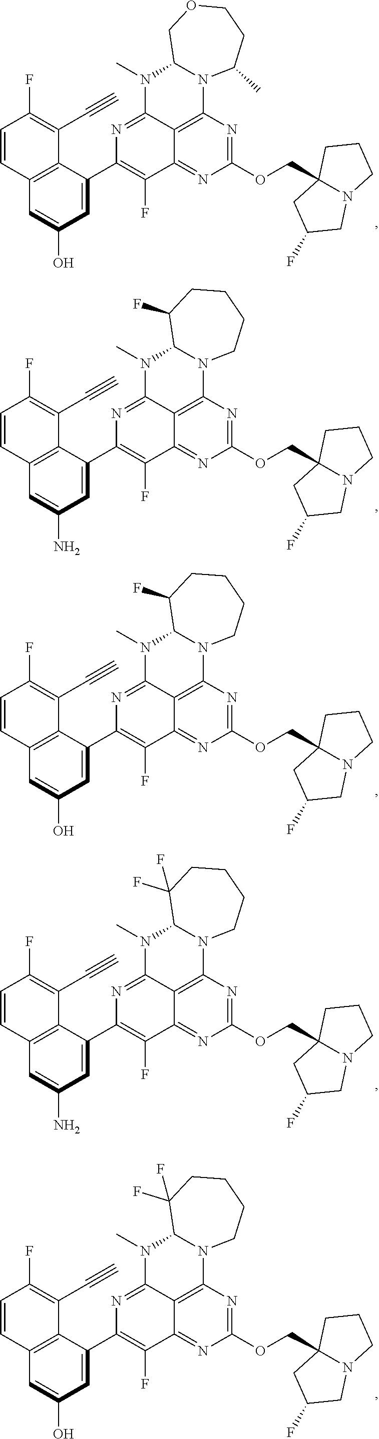

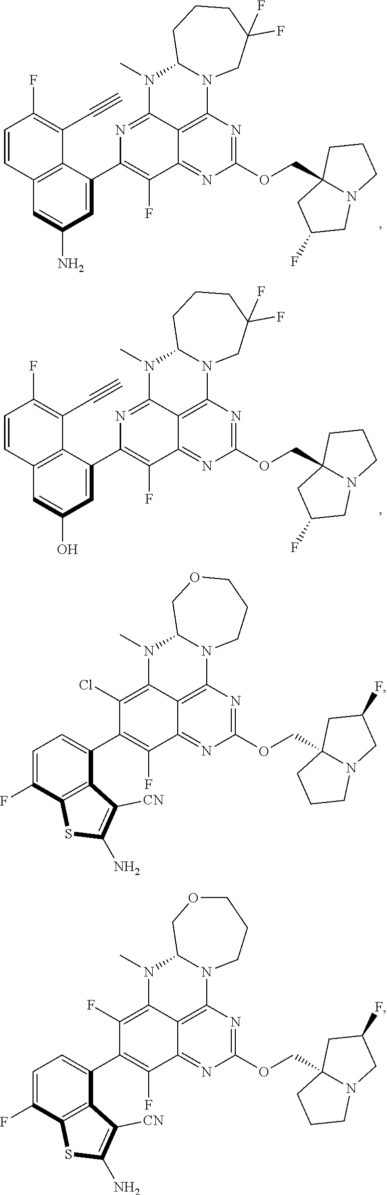

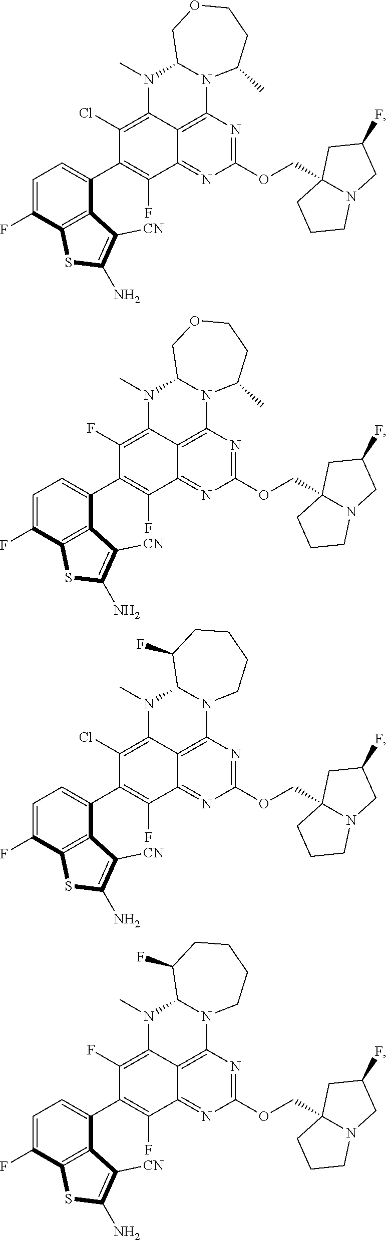

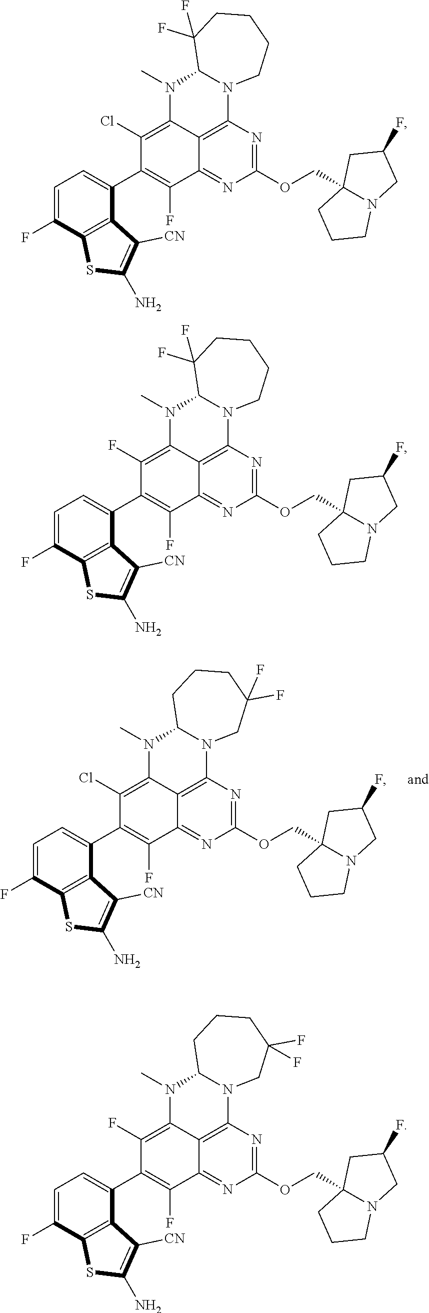

21. A pharmaceutical composition, comprising a therapeutically effective amount of the compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of claim 1, and a pharmaceutically acceptable excipient.

22. A method for treating cancer in a subject comprising administering a therapeutically effective amount of the compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of claim 1 to a subject in need thereof.

23. The method of claim 22, wherein, said cancer is pancreatic carcinoma, colorectal carcinoma, lung carcinoma, breast carcinoma, large intestine carcinoma, stomach carcinoma, endometrial carcinoma, esophageal carcinoma or gastroesophageal junction carcinoma.

24. The method of claim 23, wherein, the cancer is associated with at least one of K-Ras G12C, K-Ras G12D, K-Ras G12V, K-Ras G13D, K-Ras G12R, K-Ras G12S, K-Ras G12A, K-Ras Q61H mutation and/or K-Ras wild type amplification.

25. (canceled)

26. (canceled)

27. The compound of claim 1, wherein, the compound is selected from any one of the following formulas:

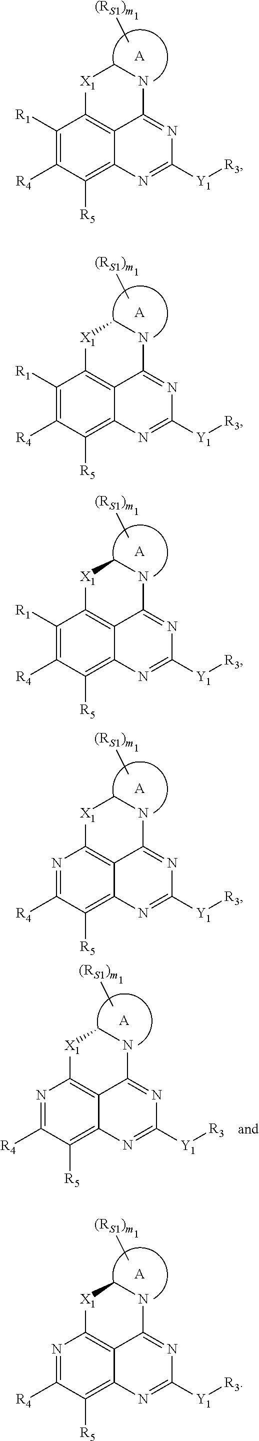

28. The method of claim 23, wherein said lung carcinoma is non-small cell lung cancer.
